SEQUENCE LISTING (3 sequences)

SEQ ID NO: 1 - protein, 404 aa, Artificial Sequence
  synthetic peptide
MAMPTTIEREFEELDTQRRWQPLYLEIRNESHDYPHRVAKFPENRNRNRYRDVSPYDHSRVKLQNAENDYINASLVDIEEAQRSYILTQGPLPNTCCHFWLMVWQQKTKAVVMLNRIVEKESVKCAQYWPTDDQEMLFKETGFSVKLLSEDVKSYYTVHLLQLENINSGETRTISHFHYTTWPDFGVPESPASFLNFLFKVRESGSLNPDHGPAVIHCSAGIGRSGTFSLVDTCLVLMEKGDDINIKQVLLNMRKYRMGLIQTPDQLRFSYMAIIEGAKCIKGDSSIQKRWKELSKEDLSPAFDHSPNKIMTEKYNGNRIGLEEEKLTGDRCTGLSSKMQDTMEENSESALRKRIREDRKATTAQKVQQMKQRLNENERKRKRPRLTDTENLYFQSHHHHHHHH

SEQ ID NO: 2 - protein, 13 aa, Artificial Sequence
  synthetic peptide
  Xaa (1)..(1): (Oregon Green)-(NH-CH2-CH2-O-CH2-CH2-O-CH2-CO)- L-threonine
  Xaa (5)..(5): O-phospho-L-tyrosine
  Xaa (13)..(13): L-lysine-NH2
XRDIXETDYYRKX

SEQ ID NO: 3 - protein, 339 aa, Artificial Sequence
  synthetic peptide
MAHHHHHHSSGLVPRGSHMEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHN

(19) United States
(12) Patent Application Publication
(10) Pub. No.: US 2024/0424111 A1
(43) Pub. Date: Dec. 26, 2024

(54) Degrader Compounds and Uses Thereof

(71) Applicants: CALICO LIFE SCIENCES LLC, South San Francisco, CA (US); ABBVIE INC., North Chicago, IL (US)

(72) Inventors: Gesine Kerstin VEITS, Watertown, MA (US); Mark E. FITZGERALD, Watertown, MA (US); Alexander W. HIRD, Watertown, MA (US); Ramzi F. SWEIS, Lake Bluff, IL (US); Michael E. KORT, Lake Bluff, IL (US)

(21) Appl. No.: 18/572,741

(22) PCT Filed: Jun. 21, 2022
(86) PCT No.: PCT/US22/34379

§ 371 Date: Dec. 20, 2023

Related U.S. Application Data
(60) Provisional application No. 63/213,086, filed on Jun. 21, 2021.

(51) Int. Cl. A61K 47/55; A61K 47/54

(52) U.S. Cl. CPC A61K 47/55; A61K 47/545

(57) ABSTRACT

Provided herein are compounds, compositions, and methods useful for degrading protein tyrosine phosphatase, e.g., protein tyrosine phosphatase non-receptor type 2 (PTPN2) and/or protein tyrosine phosphatase non-receptor type 1 (PTPN1), and for treating related diseases favorably responsive to PTPN1 or PTPN2 inhibitor treatment, e.g., a cancer or a metabolic disease.

### CROSS REFERENCE TO RELATED APPLICATIONS

This application claims the benefit of and priority to U.S. Provisional Application No. 63/213,086, filed on Jun. 21, 2021, which is incorporated herein by reference in its entirety for all purposes.

### INTRODUCTION

Cancer immunotherapy regimens targeting immune evasion mechanisms including checkpoint blockade (e.g. PD-1/PD-L1 and CTLA-4 blocking antibodies) have been shown to be effective in treating in a variety of cancers, dramatically improving outcomes in some populations refractory to conventional therapies. However, incomplete clinical responses and the development of intrinsic or acquired resistance will continue to limit the subject populations who could benefit from checkpoint blockade.

Protein tyrosine phosphatase non-receptor type 2 (PTPN2), also known as T cell protein tyrosine phosphatase (TC-PTP), is an intracellular member of the class 1 subfamily of phospho-tyrosine specific phosphatases that control multiple cellular regulatory processes by removing phosphate groups from tyrosine substrates. PTPN2 is ubiquitously expressed, but expression is highest in hematopoietic and placental cells (Mosinger, B. Jr. et al., Proc Natl Acad Sci USA 89:499-503; 1992). In humans, PTPN2 expression is controlled post-transcriptionally by the existence of two splice variants: a 45 kDa form that contains a nuclear localization signal at the C-terminus upstream of the splice junction, and a 48 kDa canonical form which has a C-terminal ER retention motif (Tillmann U. et al., Mol Cell Biol 14:3030-3040; 1994). The 45 kDa isoform can passively transfuse into the cytosol under certain cellular stress conditions. Both isoforms share an N-terminal phospho-tyrosine phosphatase catalytic domain. PTPN2 negatively regulates signaling of non-receptor tyrosine kinases (e.g. JAK1, JAK3), receptor tyrosine kinases (e.g. INSR, EGFR, CSF1R, PDGFR), transcription factors (e.g. STAT1, STAT3, STAT5a/b), and Src family kinases (e.g. Fyn, Lck). As a critical negative regulator of the JAK-STAT pathway, PTPN2 functions to directly regulate signaling through cytokine receptors, including IFNγ. The PTPN2 catalytic domain shares 74% sequence homology with PTPN1 (also called PTP1B), and shares similar enzymatic kinetics (Romsicki Y. et al., Arch Biochem Biophys 414:40-50; 2003).

Data from a loss of function in vivo genetic screen using CRISPR/Cas9 genome editing in a mouse B16F10 transplantable tumor model show that deletion of Ptpn2 gene in tumor cells improved response to the immunotherapy regimen of a GM-CSF secreting vaccine (GVAX) plus PD-1 checkpoint blockade (Manguso R. T. et al., Nature 547:413-418; 2017). Loss of Ptpn2 sensitized tumors to immunotherapy by enhancing IFNγ-mediated effects on antigen presentation and growth suppression. The same screen also revealed that genes known to be involved in immune evasion, including PD-L1 and CD47, were also depleted under immunotherapy selective pressure, while genes involved in the IFNγ signaling pathway, including IFNGR, JAK1, and STAT1, were enriched. These observations point to a putative role for therapeutic strategies that enhance IFNγ sensing and signaling in enhancing the efficacy of cancer immunotherapy regimens.

Protein tyrosine phosphatase non-receptor type 1 (PTPN1), also known as protein tyrosine phosphatase-1B (PTP1B), has been shown to play a key role in insulin and leptin signaling and is a primary mechanism for down-regulating both the insulin and leptin receptor signaling pathways (Kenner K. A. et al., J Biol Chem 271: 19810-19816, 1996). Animals deficient in PTP1B have improved glucose regulation and lipid profiles and are resistant to weight gain when treated with a high fat diet (Elchebly M. et al., Science 283: 1544-1548, 1999).

One approach to externally impact protein activity is by decreasing levels of a particular protein by targeted protein degradation. Protein degradation is a highly regulated and essential process that maintains cellular homeostasis. The selective identification and removal of damaged, misfolded, or excess proteins is achieved via the ubiquitin-proteasome pathway (UPP). The UPP is central to the regulation of almost all cellular processes, including antigen processing, apoptosis, biogenesis of organelles, cell cycling, DNA transcription and repair, differentiation and development, immune response and inflammation, neural and muscular degeneration, morphogenesis of neural networks, modulation of cell surface receptors, ion channels and the secretory pathway, the response to stress and extracellular modulators, ribosome biogenesis and viral infection.

Covalent attachment of multiple ubiquitin molecules facilitated by an E3 ubiquitin ligase to a terminal lysine residue marks the protein for proteasome degradation, where the protein is digested into small peptides and eventually into its constituent amino acids that serve as building blocks for new proteins. There are over 600 E3 ubiquitin ligases which facilitate the ubiquitination of different proteins in vivo, which can be divided into four families: HECT-domain E3s, U-box E3s, monomeric RING E3s and multi-subunit E3s. See generally Li et al. (PLOS One, 2008, 3, 1487); Berndsen et al. (Nat. Struct. Mol. Biol., 2014, 21, 301-307); Deshaies et al. (Ann. Rev. Biochem., 2009, 78, 399-434); Spratt et al. (Biochem. 2014, 458, 421-437); and Wang et al. (Nat. Rev. Cancer., 2014, 14, 233-347).

The first E3 ligase successfully targeted with a small molecule was SCF^{βTrCP}, using a hybrid of the small molecule MetAP2 inhibitor linked to a IκBα phosphopeptide epitope known to bind to the ubiquitin E3 ligase. (Sakamoto et al, PNAS 2001, 98 (15) 8554). Schneekloth et al. describe a degradation agent (PROTAC3) that targets the FK506 binding protein (FKBP12) and shows that both PROTAC2 and PROTAC3 hit their respective targets with green fluorescent protein (GFP) imaging. Schneekloth et al. (Chem Bio Chem 2005, 6, 40-46).

In unrelated parallel research, scientists were investigating thalidomide toxicity, and discovered that cereblon is a thalidomide binding protein. Ito et al. (Science 2010, 327, 1345-1350). Cereblon forms part of an E3 ubiquitin ligase protein complex which interacts with damaged DNA binding protein 1, forming an E3 ubiquitin ligase complex with Cullin 4 and the E2-binding protein ROC1 (also known as RBX1) where it functions as a substrate receptor to select proteins for ubiquitination. The study revealed that thalidomide-cereblon binding in vivo may be responsible for thalidomide teratogenicity. After the discovery that thalidomide binds to the cereblon E3 ubiquitin ligase led to research to investigate incorporating thalidomide and certain derivatives into compounds for the targeted destruction of proteins. See G. Lu et al., (Science, 343, 305-309 (2014)); and J. Kronke et al., (Science, 343, 301-305 (2014)).

While progress has been made in the area of modulation of the UPP for in vivo protein degradation, it would be useful to have additional compounds and approaches to more fully harness the UPP for therapeutic treatments, for example, for the development of targeted PTP1B degraders useful for the treatment of type 2 diabetes, obesity, and metabolic syndrome. It is an object of the present disclosure to provide new compounds, methods, compositions, and methods of manufacture that are useful to degrade selected proteins, e.g., PTP1B, in vivo.

### SUMMARY

The present disclosure is directed, at least in part, to compounds, compositions, and methods that cause degradation of a protein tyrosine phosphatase, e.g., protein tyrosine phosphatase non-receptor type 2 (PTPN2) and/or protein tyrosine phosphatase non-receptor type 1 ((PTPN1), also known as protein tyrosine phosphatase-1B (PTP1B) via the ubiquitin proteasome pathway (UPP). In some embodiments, the compounds described herein comprise a “Targeting Ligand” that binds to a protein tyrosine phosphatase, a “Degron” which binds (e.g., non-covalently) to an E3 Ligase (e.g., the cereblon component) and a linker that covalently links the Targeting Ligand to the Degron.

Some embodiments provide a compound of Formula (I):

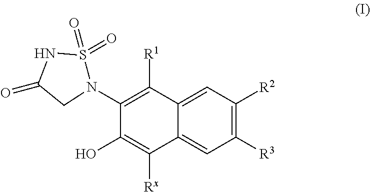

or a pharmaceutically acceptable salt thereof, wherein: R¹; R²; R³; R⁴; R⁵; R⁶; R⁷; R⁸; R⁹; R¹⁰; R^{A}; R^{B}; R; L; U; V; W; X; Y; Z; Q; p; and q are as defined herein.

Some embodiments provide a pharmaceutical composition comprising the compound of Formula (I), or a pharmaceutically acceptable salt thereof.

Some embodiments provide a method of treating cancer in a subject in need thereof, comprising administering to the subject a therapeutically effective amount of a compound of Formula (I), or a pharmaceutically acceptable salt thereof.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and figures, and from the claims.

### BRIEF DESCRIPTION OF THE SEQUENCE LISTING

Incorporated herein by reference in its entirety is a Sequence Listing entitled, “LISTING”, comprising SEQ ID NO: 1 through SEQ ID NO: 3, which includes the amino acid sequences disclosed herein. The Sequence listing has been submitted herewith in ASCII text format via EFS. The Sequence Listing was first created on Dec. 19, 2019 and is 7.25 KB in size.

### DETAILED DESCRIPTION

The present disclosure is directed, at least in part, to compounds, compositions, and methods for the inhibition of protein tyrosine phosphatase, e.g., protein tyrosine phosphatase non-receptor type 2 (PTPN2) and/or protein tyrosine phosphatase non-receptor type 1 (PTPN1 or PTP1B).

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E.L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example “C1-C6 alkyl” is intended to encompass, C1, C2, C3, C4, C5, C6, C1-C6, C1-C5, C1-C4, C1-C3, C1-C2, C2-C6, C2-C5, C2-C4, C2-C3, C3-C6, C3-C5, C3-C4, C4-C6, C4-C5, and C5-C6 alkyl.

The following terms are intended to have the meanings presented therewith below and are useful in understanding the description and intended scope of the present disclosure.

“Alkyl” refers to a radical of a straight-chain or branched saturated hydrocarbon group having from 1 to 10 carbon atoms (“C1-C10 alkyl”). In some embodiments, an alkyl group has 1 to 8 carbon atoms (“C1-C8 alkyl”). In some embodiments, an alkyl group has 1 to 6 carbon atoms (“C1-C6 alkyl”). In some embodiments, an alkyl group has 1 to 5 carbon atoms (“C1-C5 alkyl”). In some embodiments, an alkyl group has 1 to 4 carbon atoms (“C1-C4 alkyl”). In some embodiments, an alkyl group has 1 to 3 carbon atoms (“C1-C3 alkyl”). In some embodiments, an alkyl group has 1 to 2 carbon atoms (“C1-C2 alkyl”). In some embodiments, an alkyl group has 1 carbon atom (“C1 alkyl”). In some embodiments, an alkyl group has 2 to 6 carbon atoms (“C2-C6 alkyl”). Examples of C1-C6 alkyl groups include methyl (C1), ethyl (C2), n-propyl (C3), isopropyl (C3), n-butyl (C4), tert-butyl (C4), sec-butyl (C4), iso-butyl (C4), n-pentyl (C5), 3-pentanyl (C5), amyl (C5), neopentyl (C5), 3-methyl-2-butanyl (C5), tertiary amyl (C5), and n-hexyl (C6). Additional examples of alkyl groups include n-heptyl (C7), n-octyl (C8) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C1-C10 alkyl (e.g., —CH₃). In certain embodiments, the alkyl group is substituted C1-C6 alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

“Alkenyl” refers to a radical of a straight-chain or branched hydrocarbon group having from 2 to 10 carbon atoms, one or more carbon-carbon double bonds, and no triple bonds (“C2-C10 alkenyl”). In some embodiments, an alkenyl group has 2 to 8 carbon atoms (“C2-C8 alkenyl”). In some embodiments, an alkenyl group has 2 to 6 carbon atoms (“C2-C6 alkenyl”).

In some embodiments, an alkenyl group has 2 to 5 carbon atoms (“C2-C5 alkenyl”). In some embodiments, an alkenyl group has 2 to 4 carbon atoms (“C2-C4 alkenyl”). In some embodiments, an alkenyl group has 2 to 3 carbon atoms (“C2-C3 alkenyl”). In some embodiments, an alkenyl group has 2 carbon atoms (“C2 alkenyl”). The one or more carbon-carbon double bonds can be internal (such as in 2-butenyl) or terminal (such as in 1-butenyl). Examples of C2-C4 alkenyl groups include ethenyl (C2), i-propenyl (C3), 2-propenyl (C3), 1-butenyl (C4), 2-butenyl (C4), butadienyl (C4), and the like. Examples of C2-C6 alkenyl groups include the aforementioned C2-4 alkenyl groups as well as pentenyl (C5), pentadienyl (C5), hexenyl (C6), and the like. Additional examples of alkenyl include heptenyl (C7), octenyl (C8), octatrienyl (C8), and the like. Each instance of an alkenyl group may be independently optionally substituted, e.g., unsubstituted (an “unsubstituted alkenyl”) or substituted (a “substituted alkenyl”) with one or more substituents, e.g., from 1 to 5 substituents, 1 to 3 substituents, or 1 substituent. In certain embodiments, the alkenyl group is unsubstituted C2-C10 alkenyl. In certain embodiments, the alkenyl group is substituted C2-C6 alkenyl.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 10 carbon atoms, with those groups having 6 or fewer carbon atoms being preferred in the present disclosure. The term “alkenylene,” by itself or as part of another substituent, means, unless otherwise stated, a divalent radical derived from an alkene. Alkylene groups can be straight chain or branched. An alkylene group may be described as, e.g., a C1-C6 alkylene, which describes an alkylene moiety having between one and six carbon atoms.

“Halo” or “halogen,” independently or as part of another substituent, means a fluorine (F), chlorine (Cl), bromine (Br), or iodine (I) atom. The term “halide” by itself or as part of another substituent, refers to a fluoride, chloride, bromide, or iodide atom. In certain embodiments, the halo group is either fluorine or chlorine.

“Haloalkyl” refers to an alkyl group as described herein (e.g., a C1-C6 alkyl group) in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, chloro-fluoroalkyl, chloro-difluoroalkyl, and 2-fluoroisobutyl.

“Alkoxy” refers to an alkyl group as described herein (e.g., a C1-C6 alkyl group), which is attached to a molecule via oxygen atom. This includes moieties where the alkyl part may be linear or branched, such as methoxy, ethoxy, n-propoxy, iso-propoxy, n-butoxy, sec-butoxy, tert-butoxy, n-pentoxy and n-hexoxy.

“Haloalkoxy” refers to an alkoxy group as described herein (e.g., a C1-C6 alkoxy group), in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, chloro-fluoroalkoxy, chloro-difluoroalkoxy, and 2-fluoroisobutoxy.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C6-C14 aryl”). In some embodiments, an aryl group has six ring carbon atoms (“C6 aryl”; e.g., phenyl). In some embodiments, an aryl group has ten ring carbon atoms (“C10 aryl”; e.g., naphthyl such as 1-naphthyl and 2-naphthyl). In some embodiments, an aryl group has fourteen ring carbon atoms (“C14 aryl”; e.g., anthracyl). An aryl group may be described as, e.g., a C6-C10 aryl. Aryl groups include, but are not limited to, phenyl, naphthyl, indenyl, and tetrahydronaphthyl. Each instance of an aryl group may be independently optionally substituted, e.g., unsubstituted (an “unsubstituted aryl”) or substituted (a “substituted aryl”) with one or more substituents. In certain embodiments, the aryl group is unsubstituted C6-C14 aryl. In certain embodiments, the aryl group is substituted C6-C14 aryl.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl and pyridonyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

An “arylene” and a “heteroarylene,” alone or as part of another substituent, mean a divalent radical derived from an aryl and heteroaryl, respectively. Non-limiting examples of heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted as described herein, and divalent radicals of each heteroaryl example above are non-limiting examples of heteroarylene.

“Aryloxy” refers to an aryl group as described herein (e.g., a C6-C10 aryl group), which is attached to a molecule via oxygen atom. This includes, but it not limited to, groups such as phenoxy and naphthoxy.

“Heteroaryloxy” refers to a heteroaryl group as described herein (e.g., a 5 to 10 membered heteroaryl group), which is attached to a molecule via oxygen atom. This includes, but it not limited to, groups such as pyridinoxy and pyrazinoxy.

“Cycloalkyl” refers to a radical of a saturated or partially unsaturated (i.e., non-aromatic) cyclic hydrocarbon group having from 3 to 10 ring carbon atoms (“C3-C10 cycloalkyl”) and zero heteroatoms in the non-aromatic ring system. In some embodiments, a cycloalkyl group has 3 to 8 ring carbon atoms (“C3-C8 cycloalkyl”). In some embodiments, a cycloalkyl group has 3 to 6 ring carbon atoms (“C3-C6 cycloalkyl”). In some embodiments, a cycloalkyl group has 3 to 6 ring carbon atoms (“C3-C6 cycloalkyl”). In some embodiments, a cycloalkyl group has 5 to 10 ring carbon atoms (“C5-C10 cycloalkyl”). A cycloalkyl group may be described as, e.g., a C4-C7-membered cycloalkyl. Exemplary C3-C6 cycloalkyl groups include, without limitation, cyclopropyl (C3), cyclopropenyl (C3), cyclobutyl (C4), cyclobutenyl (C4), cyclopentyl (C5), cyclopentenyl (C5), cyclohexyl (C6), cyclohexenyl (C6), cyclohexadienyl (C6), and the like. Exemplary C3-C8 cycloalkyl groups include, without limitation, the aforementioned C3-C6 cycloalkyl groups as well as cycloheptyl (C7), cycloheptenyl (C7), cycloheptadienyl (C7), cycloheptatrienyl (C7), cyclooctyl (C8), cyclooctenyl (C8), cubanyl (C8), bicyclo[1.1.1]pentanyl (C5), bicyclo[2.2.2]octanyl (C8), bicyclo[2.1.1]hexanyl (C6), bicyclo[3.1.1]heptanyl (C7), and the like. Exemplary C3-C10 cycloalkyl groups include, without limitation, the aforementioned C3-C8 cycloalkyl groups as well as cyclononyl (C9), cyclononenyl (C9), cyclodecyl (C10), cyclodecenyl (C10), octahydro-1H-indenyl (C9), decahydronaphthalenyl (C10), spiro[4.5]decanyl (C10), and the like. As the foregoing examples illustrate, in certain embodiments, the cycloalkyl group is either monocyclic (“monocyclic cycloalkyl”) or contain a fused, bridged, or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, e.g., unsubstituted (an “unsubstituted cycloalkyl”) or substituted (a “substituted cycloalkyl”) with one or more substituents. In certain embodiments, the cycloalkyl group is unsubstituted C3-C10 cycloalkyl. In certain embodiments, the cycloalkyl group is a substituted C3-C10 cycloalkyl.

In some embodiments, “cycloalkyl” is a monocyclic or bicyclic, saturated or partially unsaturated group having from 3 to 10 ring carbon atoms (“C3-C10 cycloalkyl”). In some embodiments, a cycloalkyl group has 3 to 8 ring carbon atoms (“C3-C8 cycloalkyl”). In some embodiments, a cycloalkyl group has 3 to 6 ring carbon atoms (“C3-C6 cycloalkyl”). In some embodiments, a cycloalkyl group has 5 to 6 ring carbon atoms (“C5-C6 cycloalkyl”). In some embodiments, a cycloalkyl group has 5 to 10 ring carbon atoms (“C5-C10 cycloalkyl”). Examples of C5-C6 cycloalkyl groups include cyclopentyl and cyclopentenyl (C5) and cyclohexyl and cyclohexenyl (C6). Examples of C3-C6 cycloalkyl groups include the aforementioned C5-C6 cycloalkyl groups as well as cyclopropyl (C3) and cyclobutyl (C4). Examples of C3-C8 cycloalkyl groups include the aforementioned C3-C6 cycloalkyl groups as well as cycloheptyl (C7) and cyclooctyl (C8). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C3-C10 cycloalkyl. In certain embodiments, the cycloalkyl group is substituted C3-C10 cycloalkyl.

“Heterocyclyl” refers to a radical of a 3- to 12-membered saturated or partially unsaturated (i.e., non-aromatic) ring system having ring carbon atoms and 1 to 4 ring heteroatomic groups, wherein each heteroatomic group is independently selected from nitrogen, oxygen, sulfur, boron, phosphorus, and silicon (“3-12 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon (including oxo groups), nitrogen, oxygen, and sulfur and oxidized forms of sulfur (for example, S, S(O) and S(O)₂), within the moiety. Each instance of heterocyclyl may be independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-12 membered heterocyclyl. In certain embodiments, the heterocyclyl group is substituted 3-12 membered heterocyclyl. In certain embodiments, the heterocyclyl group is substituted 4-6 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, pyrrolidon-2-yl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C6 aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

A “cycloalkylene” and a “heterocyclylene,” alone or as part of another substituent, mean a divalent radical derived from a cycloalkyl and heterocyclyl, respectively. The examples above may be substituted or unsubstituted as described herein, and divalent radicals of each heterocyclyl example above are non-limiting examples of heterocyclylene and divalent radicals of each cycloalkyl example above are non-limiting examples of cycloalkylene.

“Cycloalkoxy” refers to a cycloalkyl group as described herein (e.g., a C3-C6 cycloalkyl group), which is attached to a molecule via oxygen atom. This includes, but it not limited to, groups such as cyclopropoxy, cyclobutoxy, cyclopentoxy, and cyclohexoxy.

“Heterocyclyloxy” refers to a heterocyclyl group as described herein (e.g., a 4 to 8 membered heterocyclyl group), which is attached to a molecule via oxygen atom. This includes, but it not limited to, groups such as azetidinyloxy, oxetanyloxy, piperidinyloxy, and piperazinyloxy.

“Halocycloalkoxy” refers to a cycloalkoxy group as described herein (e.g., a C3-C6 cycloalkoxy group), in which one or more of the hydrogen atoms are replaced by a halogen (e.g., mono-halocycloalkoxy, di-halocycloalkoxy, tri-halocycloalkoxy, and tetra-halocycloalkoxy). Such groups include but are not limited to, fluorocyclobutoxy, difluorocyclopentoxy, tetrafluorocyclobutoxy, chloro-fluorocycloalkoxy, chloro-difluorocycloalkoxy, and difluorocyclohexoxy.

“Amino” refers to the radical —NH₂.

“Cyano” refers to the radical —CN.

“Hydroxy” or “hydroxyl” refers to the radical —OH.

“Oxo” refers to a ═O) group.

In some embodiments one or more of the nitrogen atoms of a disclosed compound if present are oxidized to the corresponding N-oxide.

As used herein, when a ring is described as being “partially unsaturated”, it means the ring has one or more double or triple bonds between constituent ring atoms, provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

The term “pharmaceutically acceptable salts” is meant to include salts that are prepared with relatively nontoxic acids or bases, depending on the particular substituents found on the compounds described herein.

Certain compounds described herein can exist in unsolvated forms as well as solvated forms, including hydrated forms. In general, the solvated forms are equivalent to unsolvated forms and are encompassed within the scope of the present disclosure.

Certain compounds described herein possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present disclosure. The compounds described herein do not include those which are known in art to be too unstable to synthesize and/or isolate. The present disclosure includes compounds in racemic and optically pure forms. Optically active (R)- and (S)-, or (D)- and (L)-isomers may be prepared using chiral synthons or chiral reagents or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer. An example of a tautomeric forms includes the following example:

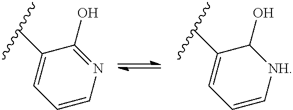 [SMILES: *c1cccnc1O ; *C1=CC=CNC1O]

It will be apparent to one skilled in the art that certain compounds of this disclosure may exist in tautomeric forms, all such tautomeric forms of the compounds being within the scope of the disclosure.

Compounds provided herein may also contain unnatural proportions of atomic isotopes at one or more of the atoms that constitute such compounds. That is, an atom, in particular when mentioned in relation to a compound according to Formula (I), comprises all isotopes and isotopic mixtures of that atom, either naturally occurring or synthetically produced, either with natural abundance or in an isotopically enriched form. For example, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof; when nitrogen is mentioned, it is understood to refer to ¹³N, ¹⁴N, ¹⁵N or mixtures thereof; when oxygen is mentioned, it is understood to refer to ¹⁴O, ¹⁵O, ¹⁶O, ¹⁷O, ¹⁸O or mixtures thereof; and when fluoro is mentioned, it is understood to refer to ¹⁸F, ¹⁹F or mixtures thereof; unless expressly noted otherwise. For example, in deuteroalkyl and deuteroalkoxy groups, where one or more hydrogen atoms are specifically replaced with deuterium (²H). As some of the aforementioned isotopes are radioactive. the compounds provided herein therefore also comprise compounds with one or more isotopes of one or more atoms, and mixtures thereof, including radioactive compounds, wherein one or more non-radioactive atoms has been replaced by one of its radioactive enriched isotopes. Radiolabeled compounds are useful as additional agents, e.g., therapeutic agents, research reagents, e.g., assay reagents, and diagnostic agents, e.g., in vivo imaging agents. All isotopic variations of the compounds provided herein, whether radioactive or not, are intended to be encompassed within the scope of the present disclosure. For example, in some embodiments, one or more C—H groups in the naphthyl ring shown in Formula (I) are replaced with C-D groups.

In the compounds described herein, it is understood that the linker group L does not include compounds, for example, where U and V; V and W; or U, V, and W; are all heteroatoms (e.g., —O—).

“Treating” or “treatment” refers to reducing the symptoms or arresting or inhibiting further development of the disease (in whole or in part). “Treating” or “treatment” includes any effect, e.g., lessening, reducing, modulating, or eliminating, that results in the improvement of the disease and the like. For example, certain methods herein treat cancer by decreasing or reducing the occurrence, growth, metastasis, or progression of cancer or decreasing a symptom of cancer.

An “effective amount” is an amount sufficient to accomplish a stated purpose (e.g. achieve the effect for which it is administered, treat a disease, reduce enzyme activity, increase enzyme activity, or reduce one or more symptoms of a disease). An example of an “effective amount” is an amount sufficient to contribute to the treatment, prevention, or reduction of a symptom or symptoms of a disease, which could also be referred to as a “therapeutically effective amount.” A “prophylactically effective amount” of a drug is an amount of a drug that, when administered to a subject, will have the intended prophylactic effect, e.g., preventing or delaying the onset (or reoccurrence) of a disease, or reducing the likelihood of the onset (or reoccurrence) of a disease or its symptoms.

A “reduction” of a symptom or symptoms means decreasing of the severity or frequency of the symptom(s), or the complete elimination of the symptom(s).

“Contacting” refers to the process of allowing at least two distinct species to become sufficiently proximal to react, interact, and/or physically touch. It should be appreciated, however, that the resulting reaction product can be produced directly from a reaction between the added reagents or from an intermediate from one or more of the added reagents which can be produced in the reaction mixture. The term “contacting” includes allowing two species to react, interact, and/or physically touch, wherein the two species may be a compound as described herein and a protein or enzyme, e.g., a protein tyrosine phosphatase, e.g., protein tyrosine phosphatase non-receptor type 2 (PTPN2) or protein tyrosine phosphatase non-receptor type 1 (PTP1B).

As defined herein, the term “inhibition”, “inhibit”, “inhibiting” and the like in reference to a protein-inhibitor (e.g., antagonist) interaction means negatively affecting (e.g., decreasing) the activity or function of the protein relative to the activity or function of the protein in the absence of the inhibitor. In some embodiments, inhibition refers to reduction in the progression of a disease and/or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein. In some embodiments, inhibition refers to a decrease in the activity of a protein tyrosine phosphatase, e.g., protein tyrosine phosphatase non-receptor type 2 (PTPN2) or protein tyrosine phosphatase non-receptor type 1 (PTP1B). Thus, inhibition may include, at least in part, partially or totally decreasing stimulation, decreasing or reducing activation, or inactivating, desensitizing, or down-regulating signal transduction or enzymatic activity or the amount of a protein tyrosine phosphatase, e.g., protein tyrosine phosphatase non-receptor type 2 (PTPN2) or protein tyrosine phosphatase non-receptor type 1 (PTP1B).

A “subject,” as used herein, refers to a living organism suffering from or prone to a disease that can be treated by administration of a compound or pharmaceutical composition, as provided herein. Non-limiting examples include mammals such as humans. In some embodiments, a subject is human. In some embodiments, a subject is a newborn human. In some embodiments, a subject is an elderly human. In some embodiments, the subject is a pediatric subject (e.g., a subject 21 years of age or less).

“Disease” refers to a state of being or health status of a subject or subject capable of being treated with a compound, pharmaceutical composition, or method provided herein. In some embodiments, the compounds and methods described herein comprise reduction or elimination of one or more symptoms of the disease, e.g., through administration of a compound described herein, a pharmaceutically acceptable salt thereof, or a pharmaceutical composition comprising a compound described herein, or a pharmaceutically acceptable salt thereof.

The term “PTPN2” as used herein refers to protein tyrosine phosphatase non-receptor type 2.

The term “PTPN1” refers to protein tyrosine phosphatase non-receptor type 1 (PTPN1), also known as protein tyrosine phosphatase-1B (PTP1B).

### Compounds

Some embodiments provide a compound of Formula (I):

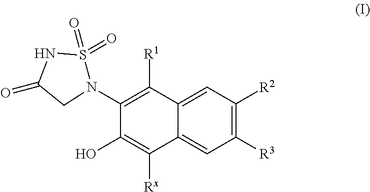

or a pharmaceutically acceptable salt thereof, wherein:
R¹ is hydrogen or halogen;
R² is hydrogen, halogen, C1-C3 alkoxy, C3-C6 cycloalkoxy, C1-C3 haloalkoxy, C3-C5 halocycloalkoxy, C1-C3 alkyl, C1-C3 haloalkyl, C3-C6 cycloalkyl, or -L-Z;
R³ is hydrogen, halogen, C1-C3 alkoxy, C3-C5 cycloalkoxy, C1-C3 haloalkoxy, C3-C5 halocycloalkoxy, C1-C3 alkyl, C1-C3 haloalkyl, C3-C5 cycloalkyl, or -L-Z;
wherein one of R² and R³ is -L-Z and the other of R² and R³ is not -L-Z;
R^{x} is hydrogen or halogen;
L is —U—V—W—X—Y—;
U is a bond, —(NR⁴)—, —O—, C1-C3 alkylene, C2-C3 alkenylene, C2-C3 alkynylene, C3-C6 cycloalkylene, 4-10 membered heterocyclylene, 5-10 membered heteroarylene, —(C═O)NR⁴—, —NR⁴(C═O)—, —OR⁵—, —R⁵O—, —NR⁴R⁵—, —R⁵NR⁴—, or —(NR⁴)(C═O)(NR⁴)—;
each R⁴ is independently a hydrogen, C1-C6 alkyl, or C3-C5 cycloalkyl;
R⁵ is C1-C3 alkylene, C3-C7 cycloalkylene, or 4-12 membered heterocyclylene;
V is a bond, —(NR⁴)—, —O—, C1-C6 alkylene, C2-C6 alkenylene, —(C═O)NR⁴—, —(NR⁴)R⁵—, —(NR⁴)(C═O)—, —NH(C═O)NH—, —OR⁵—, —R⁵O—, 4-10-membered heterocyclylene, 5-10 membered heteroarylene, C6-C10 arylene, or C3-C6 cycloalkylene;
W is a bond, C1-C3 alkylene optionally substituted with hydroxyl, C3-C6 cycloalkylene, 4-12 membered heterocyclylene, —O—, —(NR⁴)—, —R⁵(NR⁴)—, —(NR⁴)R⁵—, —(NR⁴)(C═O)—, —R⁵(NR⁴)(C═O)—, —(C═O)(NR⁴)R⁵—, —R⁵(C═O)(NR⁴)—, —(C═O)(NR⁴)—, —R⁵(C═O)—, —(C═O)R⁵—, —(C═O)—, —(S═O)—, or —S(O₂)—;
X is a bond, C1-C3 alkylene, C3-C6 cycloalkylene, 4-12 membered heterocyclylene, C6-C10 arylene, 5-10 membered heteroarylene, —R⁵(NR⁴)(C═O)—, —(C═O)R⁵(NR⁴)—, —R⁵(C═O)(NR⁴)—, —(NR⁴)(C═O)R⁵—, —R⁵(C═O)(NR⁴)—, —(C═O)(NR⁴)R⁵—, —(NR⁴)R⁵(C═O)—, —R⁵(C═O)(NR⁴)R⁵—, —R⁵(NR⁴)(C═O)R⁵—, —(C═O)R⁵—, or —R⁵(C═O)—;
Y is R⁶, —R⁶(CR^{A}R^{B})ₚ-Q-, or -Q-(CR^{A}R^{B})ₚR⁶—;
Q is selected from the group consisting of —(NR⁴)—, —O—, and —(CR^{A}R^{B})ₚ;
p is 0, 1, 2, or 3;
R⁶ is C1-C3 alkylene, C3-C7 cycloalkylene, 4-12 membered heterocyclylene, C6-C10 arylene, or 5-10 membered heteroarylene;
wherein the heterocyclylene, heteroarylene, arylene, and cycloalkylene groups of U, V, W, X, and R⁶ are each optionally substituted with 1-3 substituents independently selected from fluoro, hydroxyl, C1-C6 alkoxy, and C1-C6 alkyl;
each R^{A} and R^{B} is independently hydrogen, fluoro, or C1-C6 alkyl; or
R^{A} and R^{B}, together with the carbon atom to which they are attached, come together to form a C3-C4 cycloalkyl; or
R^{A} and R^{B} combine to form oxo;
Z is selected from the group consisting of

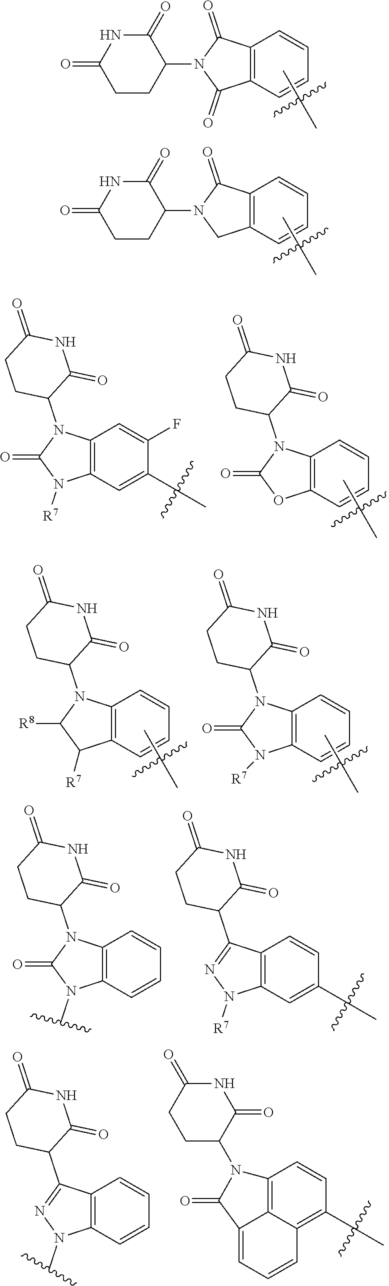

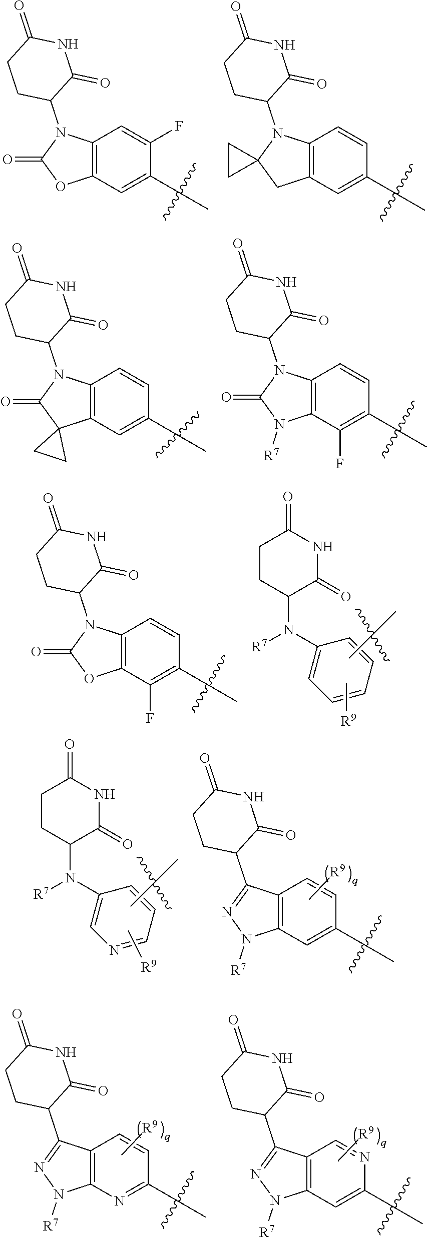

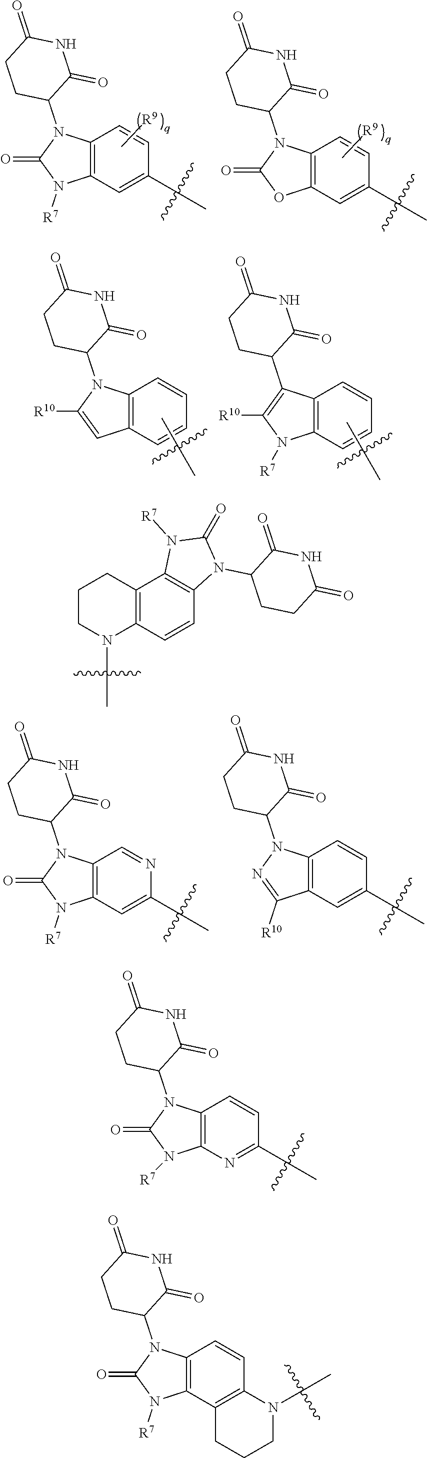

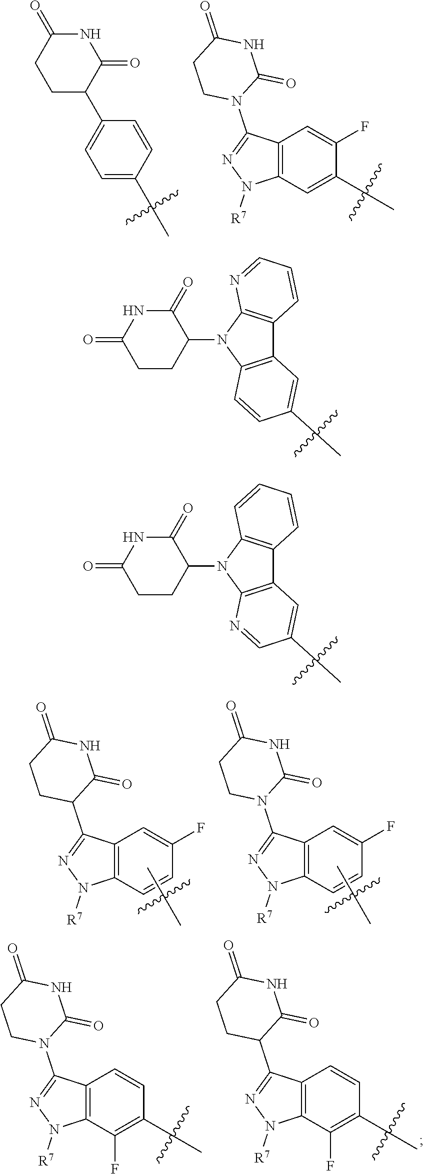

R⁷ is hydrogen, C1-C6 alkyl optionally substituted with one group selected from hydroxyl, cyano and C1-C6 alkoxy, C1-C6 haloalkyl, C3-C6 cycloalkyl, 4-6 membered heterocyclyl,
   —(CR^{A}R^{B})(4-12 membered heterocyclyl), or —(CR^{A}R^{B})(C3-C6 cycloalkyl);
R⁸ is hydrogen or C1-C6 alkyl;
each R⁹ is hydrogen, halogen, cyano, C1-C6 alkyl, C1-C6 haloalkyl, C1-C6 alkoxy, C1-C5 cycloalkoxy, 5-10 membered heteroaryloxy, or phenoxy; q is 0, 1, or 2; and
each R¹⁰ is independently hydrogen, halogen, cyano, C1-C6 alkyl, C3-C6 cycloalkyl, or C1-C6 haloalkyl.

In some embodiments, L is —U—V—W—X—Y—, wherein —Y— is, for example, the point of connection to Z; and wherein —U— is the point of connection to the remainder of Formula (I) (e.g., the naphthyl ring shown in Formula (I).

In some embodiments of a compound of Formula (I), R¹ is halogen. In some embodiments of a compound of Formula (I), R¹ is —F. In some embodiments of a compound of Formula (I), R¹ is —Cl. In some embodiments of a compound of Formula (I), R¹ is hydrogen.

In some embodiments of a compound of Formula (I), RN is halogen. In some embodiments of a compound of Formula (I), RN is —F or —Cl. In some embodiments of a compound of Formula (I), RN is hydrogen.

In some embodiments of a compound of Formula (I), R² is -L-Z.

In some embodiments of a compound of Formula (I), R³ is hydrogen. In some embodiments of a compound of Formula (I), R³ is halogen. In some embodiments of a compound of Formula (I), R³ is C1-C3 alkoxy or C1-C3 haloalkoxy. In some embodiments of a compound of Formula (I), R³ is C3-C5 cycloalkoxy or C3-C5 halocycloalkoxy. In some embodiments of a compound of Formula (I), R³ is C1-C3 alkyl or C3-C5 cycloalkyl. In some embodiments of a compound of Formula (I), R³ is C1-C3 haloalkyl.

In some embodiments of a compound of Formula (I), R² is -L-Z and R³ is hydrogen. In some embodiments of a compound of Formula (I), R² is -L-Z and R³ is halogen. In some embodiments of a compound of Formula (I), R² is -L-Z and R³ is C1-C3 alkoxy or C1-C3 haloalkoxy. In some embodiments of a compound of Formula (I), R² is -L-Z and R³ is C3-C5 cycloalkoxy or C3-C5 halocycloalkoxy. In some embodiments of a compound of Formula (I), R² is -L-Z and R³ is C1-C3 alkyl or C3-C5 cycloalkyl.

In some embodiments of a compound of Formula (I), R³ is -L-Z.

In some embodiments of a compound of Formula (I), R² is hydrogen. In some embodiments of a compound of Formula (I), R² is halogen. In some embodiments of a compound of Formula (I), R² is C1-C3 alkoxy or C1-C3 haloalkoxy. In some embodiments of a compound of Formula (I), R² is C3-C5 cycloalkoxy or C3-C5 halocycloalkoxy. In some embodiments of a compound of Formula (I), R² is C1-C3 alkyl or C3-C5 cycloalkyl. In some embodiments of a compound of Formula (I), R² is C1-C3 haloalkyl.

In some embodiments of a compound of Formula (I), R³ is -L-Z and R² is hydrogen. In some embodiments of a compound of Formula (I), R³ is -L-Z and R² is halogen. In some embodiments of a compound of Formula (I), R³ is -L-Z and R² is C1-C3 alkoxy or C1-C3 haloalkoxy. In some embodiments of a compound of Formula (I), R³ is -L-Z and R² is C3-C5 cycloalkoxy or C3-C5 halocycloalkoxy. In some embodiments of a compound of Formula (I), R³ is -L-Z and R² is C1-C3 alkyl or C3-C5 cycloalkyl.

In some embodiments of a compound of Formula (I), R¹ is —F; and RN is hydrogen, —F, or —Cl. In some embodiments of a compound of Formula (I), R¹ is —F; RN is hydrogen; R² is -L-Z; and R³ is hydrogen. In some embodiments of a compound of Formula (I), R¹ is —F; RN is hydrogen; R² is hydrogen; and R³ is -L-Z.

In some embodiments, U is a bond, —(NR⁴)—, —O—, C1-C3 alkylene, C2-C3 alkenylene, C2-C3 alkynylene, C3-C6 cycloalkylene, 4-10 membered heterocyclylene, 5-10 membered heteroarylene, —(C═O)NR⁴—, —NR⁴(C═O)—, —OR⁵—, —R⁵O—, —NR⁴R⁵—, —R⁵NR⁴—, or —(NR⁴)(C═O)(NR⁴)—. In some embodiments, U is —(NR⁴)—, —NR⁴R⁵—, or —R⁵NR⁴—. In some embodiments, U is —(NR⁴)—. In some embodiments, R⁴ is hydrogen. In some embodiments, R⁴ is C1-C6 alkyl. In some embodiments, U is —O—, —OR⁵—, or —R⁵O—. In some embodiments, U is —O—. In some embodiments, U is —NR⁴(C═O)—, —(C═O)NR⁴—, or —(NR⁴)(C═O)(NR⁴)—. In some embodiments, wherein U is —NR⁴(C═O)—. In some embodiments, each R⁴ within U is independently hydrogen or C1-C6 alkyl. In some embodiments, each R⁴ within U is hydrogen.

In some embodiments, wherein U is C1-C3 alkylene, C2-C3 alkenylene, or C2-C3 alkynylene. In some embodiments, U is C2-C3 alkenylene. In some embodiments, U is C2-C3 alkynylene. In some embodiments, U is C3-C6 cycloalkylene, 4-10 membered heterocyclylene, or 5-10 membered heteroarylene; each optionally substituted with 1-3 substituents independently selected from fluoro, hydroxyl, C1-C6 alkoxy, and C1-C6 alkyl. In some embodiments, U is a bond.

In some embodiments, V is a bond, —(NR⁴)—, —O—, C1-C6 alkylene, C2-C6 alkenylene, —(C═O)NR⁴—, —(NR⁴)R⁵—, —(NR⁴)(C═O)—, —NH(C═O)NH—, —OR⁵—, —R⁵O—, 4-10-membered heterocyclylene, 5-10 membered heteroarylene, C6-C10 arylene, or C3-C6 cycloalkylene. In some embodiments, V is C1-C6 alkylene or C2-C6 alkenylene. In some embodiments, V is C1-C6 alkylene. In some embodiments, V is C1-C3 alkylene. In some embodiments, V is methylene or ethylene.

In some embodiments, V is 4-10-membered heterocyclylene, 5-10 membered heteroarylene, C6-C10 arylene, or C3-C6 cycloalkylene; each optionally substituted with 1-3 substituents independently selected from fluoro, hydroxyl, C1-C6 alkoxy, and C1-C6 alkyl. In some embodiments, V is 4-10 membered heterocyclylene, 5-10 membered heteroarylene, C6-C10 arylene, or C3-C6 cycloalkylene; each substituted with 1-3 substituents independently selected from fluoro, hydroxyl, C1-C6 alkoxy, and C1-C6 alkyl. In some embodiments, V is 4-membered heterocyclylene, 5-10 membered heteroarylene, C6-C10 arylene, or C3-C6 cycloalkylene.

In some embodiments, V is 4-10-membered heterocyclylene. In some embodiments, V is 4-6-membered heterocyclylene. In some embodiments, V is selected from the group consisting of:

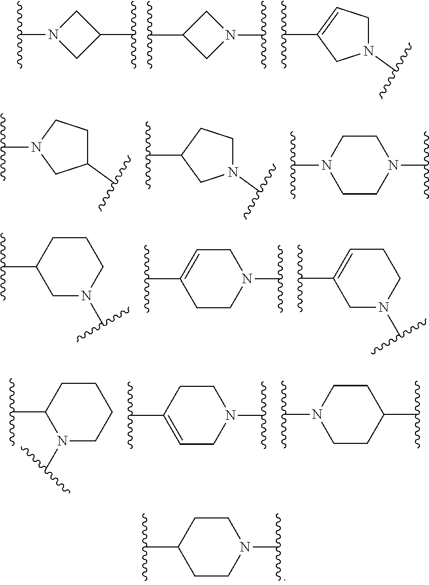

In some embodiments, V is 5-10 membered heteroarylene. In some embodiments, V is 5-6 membered heteroarylene. In some embodiments, V is selected from the group consisting of:

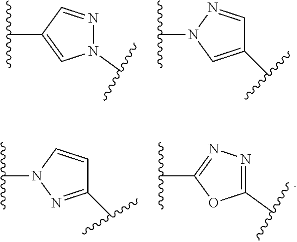 [SMILES: *c1cnn(*)c1 ; *c1cnn(*)c1 ; *c1ccn(*)n1 ; *c1nnc(*)o1]

In some embodiments, V is a C6-C10 arylene. In some embodiments, V is phenyl. In some embodiments, V is naphthyl.

In some embodiments, V is C3-C6 cycloalkylene. In some embodiments, V is selected from the group consisting of cyclobutylene, cyclopentylene, and cyclohexylene.

In some embodiments, V is —(C═O)NR⁴—, —(NR⁴)R⁵—, —(NR⁴)(C═O)—, or —NH(C═O)NH—. In some embodiments, V is —(NR⁴)— or —(NR⁴)R⁵—. In some embodiments, V is —O—, —OR⁵—, or —R⁵O—. In some embodiments, V is a bond.

In some embodiments, W is a bond, C1-C3 alkylene optionally substituted with hydroxyl, C3-C6 cycloalkylene, 4-12 membered heterocyclylene, —O—, —(NR⁴)—, —R⁵(NR⁴)—, —(NR⁴)R⁵—, —(NR⁴)(C═O)—, —R⁵(NR⁴)(C═O)—, —(C═O)(NR⁴)R⁵—, —R⁵(C═O) (NR⁴)—, —(C═O)(NR⁴)—, —R⁵(C═O)—, —(C═O)R⁵—, —(C═O)—, —(S═O)—, or —S(O₂)—.

In some embodiments, W is a bond. In some embodiments, W is C1-C3 alkylene optionally substituted with hydroxyl. In some embodiments, W is C1-C3 alkylene substituted with hydroxyl. In some embodiments, W is C1-C3 alkylene. In some embodiments, W is C3-C6 cycloalkylene or 4-12 membered heterocyclylene; each optionally substituted with 1-3 substituents independently selected from fluoro, hydroxyl, C1-C6 alkoxy, and C1-C6 alkyl. In some embodiments, W is —O—, —(NR⁴)—, —R⁵(NR⁴)—, or —(NR⁴)R⁵—. In some embodiments, W is —O— or —(NR⁴)—. In some embodiments, each R⁴ in W is hydrogen.

In some embodiments, W is —(NR⁴)(C═O)—, —R⁵(NR⁴)(C═O)—, —(C═O)(NR⁴)R⁵—, —R⁵(C═O)(NR⁴)—, or —(C═O)(NR⁴)—. In some embodiments, W is —(NR⁴)(C═O)—. In some embodiments, W is —R⁵(NR⁴)(C═O)—. In some embodiments, W is —(C═O)(NR⁴)—. In some embodiments, R⁴ within W is hydrogen. In some embodiments, each R⁴ within W is independently C1-C3 alkyl. In some embodiments, each R⁵ within W is C1-C3 alkylene. In some embodiments, W is —R⁵(C═O)—, —(C═O)R⁵—, —(C═O)—, —(S═O)—, or —S(O₂)—. In some embodiments, W is —(C═O)—. In some embodiments, W is —R⁵(C═O)— or —(C═O)R⁵—, and R⁵ is C1-C3 alkylene. In some embodiments, W is —R⁵(C═O)— or —(C═O)R⁵—, and R⁵ is C3-C7 cycloalkylene.

In some embodiments, X is a bond, C1-C3 alkylene, C3-C6 cycloalkylene, 4-12 membered heterocyclylene, C6-C10 arylene, 5-10 membered heteroarylene, —R⁵(NR⁴)(C═O)—, —(C═O)R⁵(NR⁴)—, —R⁵(C═O)(NR⁴)—, —(NR⁴)(C═O)R⁵—, —R⁵(C═O)(NR⁴)—, —(C═O)(NR⁴)R⁵—, —(NR⁴)R⁵(C═O)—, —R⁵(C═O)(NR⁴)R⁵—, —R⁵(NR⁴)(C═O)R⁵—, —(C═O)R⁵—, or —R⁵(C═O)—.

In some embodiments, X is C1-C3 alkylene. In some embodiments, X is methylene or ethylene.

In some embodiments, X is C3-C6 cycloalkylene, 4-12 membered heterocyclylene, C6-C10 arylene, or 5-10 membered heteroarylene; each optionally substituted with 1-3 substituents independently selected from fluoro, hydroxyl, C1-C6 alkoxy, and C1-C6 alkyl. In some embodiments, X is C3-C6 cycloalkylene, 4-12 membered heterocyclylene, C6-C10 arylene, or 5-10 membered heteroarylene; each substituted with 1-3 substituents independently selected from fluoro, hydroxyl, C1-C6 alkoxy, and C1-C6 alkyl. In some embodiments, X is C3-C6 cycloalkylene, 4-12 membered heterocyclylene, C6-C10 arylene, or 5-10 membered heteroarylene.

In some embodiments, X is C3-C6 cycloalkylene or 4-12 membered heterocyclylene. In some embodiments, X is 4-10 membered heterocyclylene. In some embodiments, X is 4-6 membered heterocyclylene. In some embodiments, X is selected from the group consisting of:

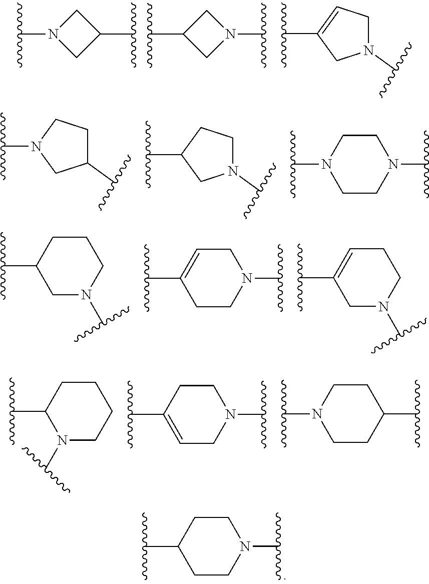

In some embodiments, X is

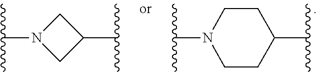 [SMILES: *C1CN(*)C1 ; *C1CCN(*)CC1]

In some embodiments, X is C3-C6 cycloalkylene, such as cyclopentyl or cyclohexyl.

In some embodiments, X is 5-10 membered heteroarylene. In some embodiments, X is 5-6 membered heteroarylene. In some embodiments, V is selected from the group consisting of:

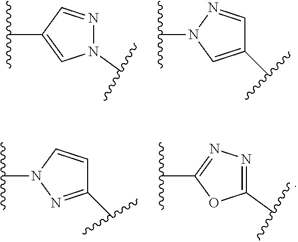 [SMILES: *c1cnn(*)c1 ; *c1cnn(*)c1 ; *c1ccn(*)n1 ; *c1nnc(*)o1]

In some embodiments, X is a C6-C10 arylene. In some embodiments, X is phenyl. In some embodiments, X is naphthyl.

In some embodiments, X is selected from the group consisting of —R⁵(NR⁴)(C═O)—, —(C═O)R⁵(NR⁴)—, —R⁵(C═O)(NR⁴)—, —(NR⁴)(C═O)R⁵—, —R⁵(C═O)(NR⁴)—, —(C═O)(NR⁴)R⁵—, —(NR⁴)R⁵(C═O)—, —R⁵(C═O)(NR⁴)R⁵—, or —R⁵(NR⁴)(C═O)R⁵—. In some embodiments, X is —(C═O)R⁵— or —R⁵(C═O)—. In some embodiments, each R⁴ within X is independently hydrogen or C1-C3 alkyl. In some embodiments, each R⁴ within X is hydrogen. In some embodiments, R⁵ is C1-C3 alkylene. In some embodiments, X is a bond.

In some embodiments, U is —NR⁴(C═O)— or —(C═O)NR⁴—; V is a bond or C1-C6 alkylene; W is a bond; and X is a bond. In some embodiments, U is —NR⁴(C═O)— or —(C═O)NR⁴—; V is a bond, C1-C6 alkylene, or C3-C6 cycloalkylene; W is a bond; and X is 4-12-membered heterocyclylene. In some embodiments, U is —NR⁴(C═O)—. In some embodiments, U is —(C═O)NR⁴—. In some embodiments, V is C3-C6 cycloalkylene. In some embodiments, V is a bond. In some embodiments, V is C1-C3 alkylene. In some embodiments, V is methylene or ethylene.

In some embodiments, U is —(NR⁴)(C═O)(NR⁴)—, —NR⁴(C═O)—, or —(C═O)NR⁴—; V is a bond, C1-C6 alkylene, or C3-C6 cycloalkylene; W is a bond; and X is a bond, C6-C10 arylene, or C1-C3 alkylene. In some embodiments, U is —(NR⁴)(C═O)(NR⁴)—.

In some embodiments, wherein U is —O—; V is C1-C6 alkylene, C3-C6 cycloalkylene, or 4-10-membered heterocyclylene; W is a bond, —R⁵(C═O)—, —(C═O)R⁵—, —C(═O)—, —N(R⁴)—, —C(═O)NR⁴—, —NR⁴C(═O)—, or —NR⁴C(═O)R⁵—. In some embodiments, V is C1-C6 alkylene. In some embodiments, V is C1-C3 alkylene. In some embodiments, V is methylene or ethylene. In some embodiments, W is —C(═O)— or —C(═O)NR⁴—. In some embodiments, W is —NR⁴C(═O)—. In some embodiments, W is —NR⁴C(═O)R⁵—. In some embodiments, each R⁴ within W is hydrogen. In some embodiments, each R⁵ within W is independently C1-C3 alkylene. In some embodiments, R⁵ is C3-C7 cycloalkylene.

In some embodiments, U is —NR⁴—, a bond, or 4-10 membered heterocyclylene; V is 4-membered heterocyclylene, C1-C6 alkylene or a bond; W is —C(═O)— or —C(═O)R⁵—; and X is a bond or C1-C3 alkylene. In some embodiments, U is —NH—. In some embodiments, U is —N(C1-C3 alkyl)-. In some embodiments, U is a bond. In some embodiments, U is 4-10 membered heterocyclylene. In some embodiments, V is 4-10 membered heterocyclylene. In some embodiments, V is C1-C3 alkylene. In some embodiments, V is methylene or ethylene. In some embodiments, W is —C(═O)—. In some embodiments, W is —C(═O)R⁵—. In some embodiments, each R⁵ within W is independently C1-C3 alkylene.

In some embodiments, U is a bond, C1-C3 alkylene, C2-C3 alkenylene, or C2-C3 alkynylene; V is a bond; W is a bond or C(═O); and X is a bond or C6-C10 arylene. In some embodiments, U is a bond. In some embodiments, U is C2-C3 alkenylene. In some embodiments, U is C2-C3 alkynylene. In some embodiments, W is a bond. In some embodiments, W is C(═O). In some embodiments, X is a bond. In some embodiments, X is C6-C10 arylene. In some embodiments, X is C1-C3 alkylene. In some embodiments, each R⁵ within W is independently C1-C3 alkylene or C3-C7 cycloalkylene.

In some embodiments, U is —NR⁴(C═O)—, —(C═O)NR⁴—, or —(NR⁴)(C═O)(NR⁴)—; V is C1-C6 alkylene; W is a bond or C1-C3 alkylene; and X is a bond. In some embodiments, U is —NR⁴(C═O)—. In some embodiments, U is —(C═O)NR⁴—. In some embodiments, U is —(NR⁴)(C═O)(NR⁴)—. In some embodiments, V is C1-C6 alkylene. In some embodiments, W is a bond. In some embodiments, W is C1-C3 alkylene. In some embodiments, W is methylene, ethylene, or propylene. In some embodiments, each R⁴ within U is hydrogen.

In some embodiments, Y is R⁶, R⁶(CR^{A}R^{B})ₚ-Q-, or -Q-(CR^{A}R^{B})ₚR⁶—.

In some embodiments, Y is R⁶. In some embodiments, R⁶ is 4-12 membered heterocyclylene optionally substituted with 1-3 substituents independently selected from fluoro, hydroxyl, C1-C6 alkoxy, and C1-C6 alkyl. In some embodiments, R⁶ is 4-8 membered heterocyclylene optionally substituted with 1-3 substituents independently selected from fluoro, hydroxyl, C1-C6 alkoxy, and C1-C6 alkyl. In some embodiments, R⁶ is 4-6 membered heterocyclylene optionally substituted with 1-3 substituents independently selected from fluoro, hydroxyl, C1-C6 alkoxy, and C1-C6 alkyl.

In some embodiments, R⁶ is 4-12 membered heterocyclylene substituted with 1-3 substituents independently selected from fluoro, hydroxyl, C1-C6 alkoxy, and C1-C6 alkyl. In some embodiments, R⁶ is 4-8 membered heterocyclylene substituted with 1-3 substituents independently selected from fluoro, hydroxyl, C1-C6 alkoxy, and C1-C6 alkyl. In some embodiments, R⁶ is 4-6 membered heterocyclylene substituted with 1-3 substituents independently selected from fluoro, hydroxyl, C1-C6 alkoxy, and C1-C6 alkyl.

In some embodiments, R⁶ is 4-8 membered heterocyclylene substituted with hydroxyl. In some embodiments, R⁶ is 4-8 membered heterocyclylene substituted with C1-C6 alkyl, such as methyl. In some embodiments, R⁶ is 4-8 membered heterocyclylene substituted with fluoro. In some embodiments, R⁶ is 4-8 membered heterocyclylene substituted with two fluoros.

In some embodiments, R⁶ is 4-12 membered heterocyclylene. In some embodiments, R⁶ is 4-8 membered heterocyclylene. In some embodiments, R⁶ is 4-6 membered heterocyclylene.

In some embodiments, R⁶ is selected from the group consisting of:

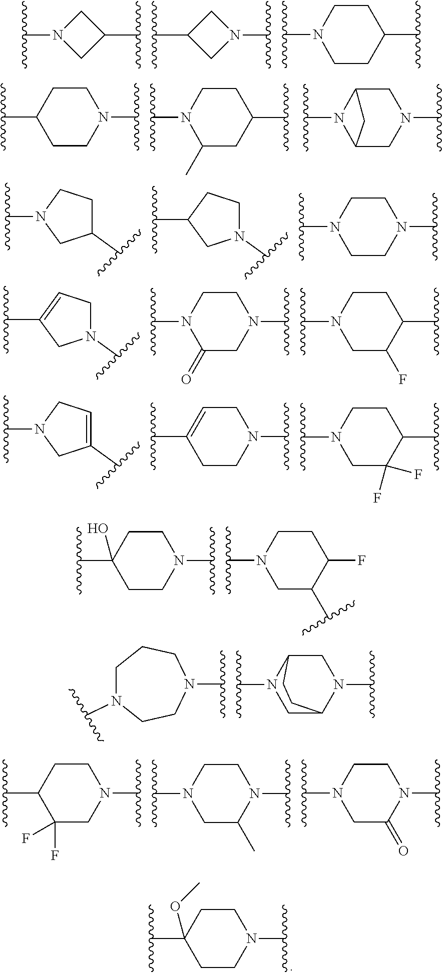

In some embodiments, R⁶ is

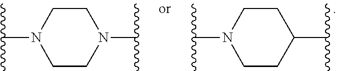 [SMILES: *N1CCN(*)CC1 ; *C1CCN(*)CC1]

In some embodiments, R⁶ is.

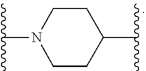 [SMILES: *C1CCN(*)CC1]

In some embodiments, R⁶ is 7-12 membered bicyclic heterocyclylene. In some embodiments, R⁶ is 7-12 membered bicyclic spirocyclic heterocyclylene. In some embodiments, R⁶ is

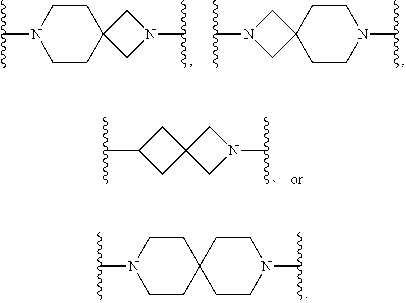

In some embodiments, R⁶ is 5-10 membered heteroarylene optionally substituted with 1-3 substituents independently selected from fluoro, hydroxyl, C1-C6 alkoxy, and C1-C6 alkyl. In some embodiments, R⁶ is 5-6 membered heteroarylene optionally substituted with 1-3 substituents independently selected from fluoro, hydroxyl, C1-C6 alkoxy, and C1-C6 alkyl. In some embodiments R⁶ is 5-6 membered heteroarylene. In some embodiments, R⁶ is selected from the group consisting of:

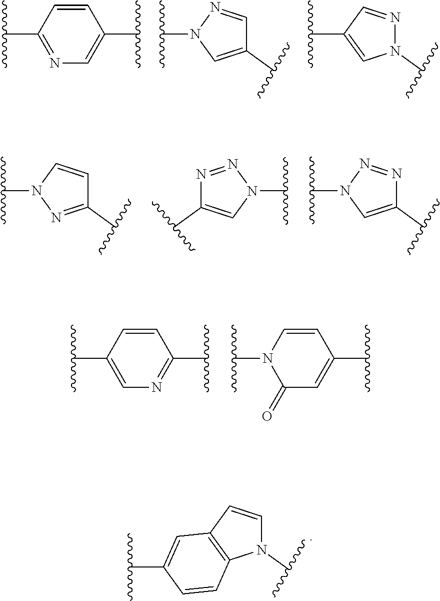

In some embodiments, R⁶ is C1-C3 alkylene.

In some embodiments, —Y— is —R⁶(CR^{A}R^{B})ₚ-Q-. In some embodiments, —Y— is -Q-(CR^{A}R^{B})ₚR⁶—. In some embodiments, -Q- is —(NR⁴)—. In some embodiments, R⁴ is hydrogen. In some embodiments, R⁴ is C1-C3 alkyl. In some embodiments, -Q- is —O—.

In some embodiments, p is 0, 1, or 2. In some embodiments, p is 0 or 1. In some embodiments, p is 1 or 2. p is 0. In some embodiments, p is 1. In some embodiments, p is 2.

In some embodiments, each R^{A} and R^{B} are independently hydrogen, fluoro, or C1-C3 alkyl. In some embodiments, one pair of R^{A} and R^{B}, on the same carbon, combine to form oxo. In some embodiments, each R^{A} and R^{B} are hydrogen. In some embodiments, 1 or 2 of R^{A} and R^{B} are independently fluoro or C1-C3 alkyl; and each remaining R^{A} and R^{B} is hydrogen. In some embodiments, one pair of R^{A} and R^{B}, on the same carbon, combine to form oxo; and each remaining R^{A} and R^{B}, if present, are hydrogen.

In some embodiments, Y is —R⁶(CR^{A}R^{B})ₚ-Q-; and p is 0. In some embodiments, Y is —R⁶NR⁴— or —R⁶O—. In some embodiments, Y is —R⁶NR⁴—. In some embodiments, Y is —R⁶O—. In some embodiments, Y is R⁶(CR^{A}R^{B})ₚ-Q- or -Q-(CR^{A}R^{B})ₚR⁶—; p is 1 or 2; and each R^{A} and R^{B} are hydrogen. In some embodiments, Y is —R⁶CH₂—O— or —R⁶CH₂—N(R⁴)—. In some embodiments, Y is —R⁶CH₂—O—. In some embodiments, Y is —R⁶CH₂—NH.

In some embodiments, Y is —R⁶(CR^{A}R^{B})ₚ-Q- or -Q-(CR^{A}R^{B})ₚR⁶—; p is 1 or 2; and each R^{A} and R^{B} are independently hydrogen or C1-C3 alkyl; or one pair of R^{A} and R^{B}, together with the carbon atom to which they are attached, come together to form a C3-C4 cycloalkyl, and each remaining R^{A} and R^{B}, if present, are hydrogen. In some embodiments, Y is —R⁶(CR^{A}R^{B})ₚ-Q-. In some embodiments, Y is -Q-(CR^{A}R^{B})ₚR⁶—.

In some embodiments, the —(CR^{A}R^{B})ₚ-Q- portion of Y is selected from the group consisting of:

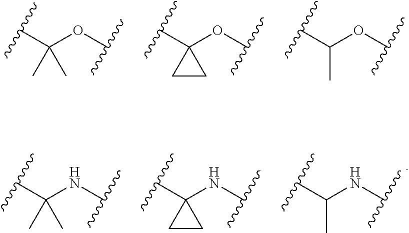

In some embodiments, Y is —R⁶C(═O)(CR^{A}R^{B})-Q-; and each R^{A} and R^{B} are independently hydrogen, fluoro, or C1-C3 alkyl. In some embodiments, Y is -Q-(CR^{A}R^{B})ₚR⁶—; and each R^{A} and R^{B} are independently hydrogen, fluoro, or C1-C3 alkyl. In some embodiments, the —(CR^{A}R^{B})ₚ-Q- portion of Y is selected from the group consisting of:

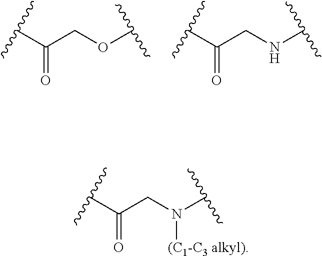

In some embodiments, R⁶ is 5-10 membered heteroarylene optionally substituted with 1-3 substituents independently selected from fluoro, hydroxyl, C1-C6 alkoxy, and C1-C6 alkyl. In some embodiments, R⁶ is 5-6 membered heteroarylene optionally substituted with 1-3 substituents independently selected from fluoro, hydroxyl, C1-C6 alkoxy, and C1-C6 alkyl. In some embodiments, R⁶ is 5-10 membered heteroarylene substituted with 1-3 substituents independently selected from fluoro, hydroxyl, C1-C6 alkoxy, and C1-C6 alkyl. In some embodiments, R⁶ is 5-6 membered heteroarylene substituted with 1-3 substituents independently selected from fluoro, hydroxyl, C1-C6 alkoxy, and C1-C6 alkyl. In some embodiments, R⁶ is 5-membered heteroarylene. In some embodiments, R⁶ is 5-6 membered heteroarylene.

In some embodiments, R⁶ is 5-6 membered heteroarylene. In some embodiments, R⁶ is triazolylene, pyrazolylene, or pyridinylene. In some embodiments, R⁶ is selected from the group consisting of:

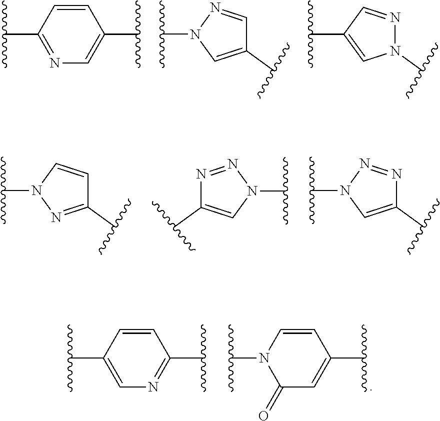

In some embodiments, R⁶ is C6-C10 arylene. In some embodiments, R⁶ is phenylene.

In some embodiments, Z is selected from the group consisting of:

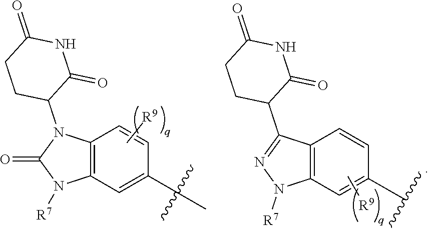

In some embodiments, Z is:

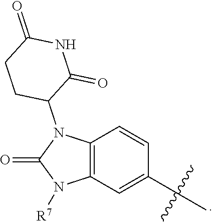

In some embodiments, Z is

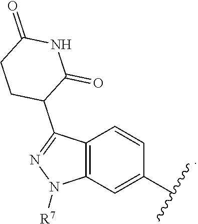

In some embodiments, Z is:

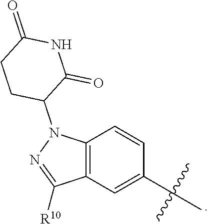

In some embodiments, Z is:

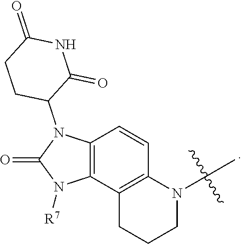

In some embodiments, Z is selected from the group consisting of:

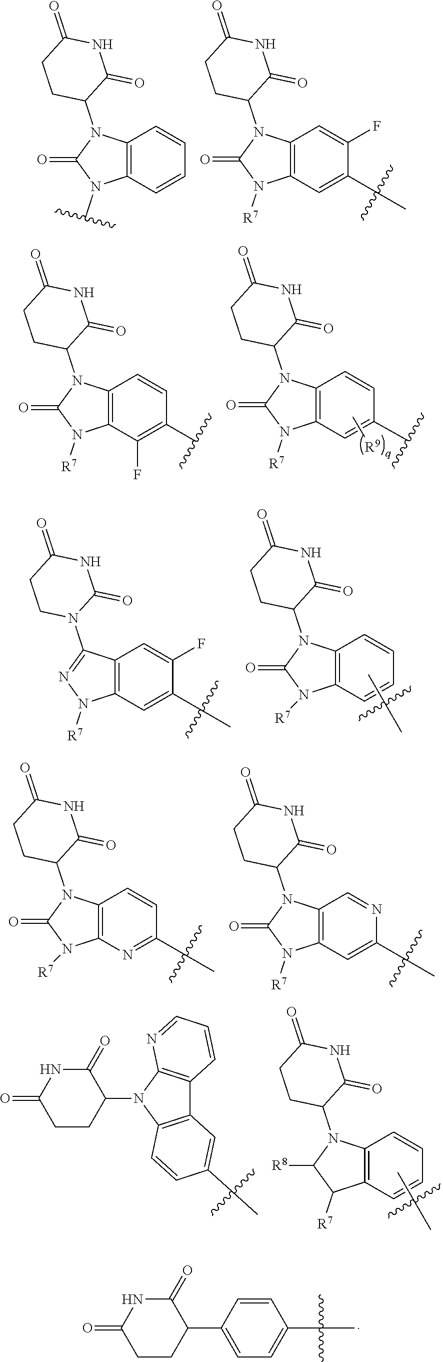

In some embodiments, Z is:

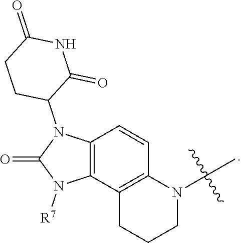

In some embodiments, Z is:

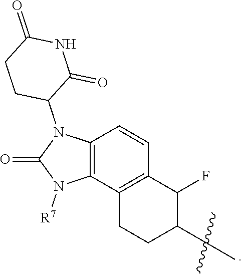

In some embodiments, Z is:

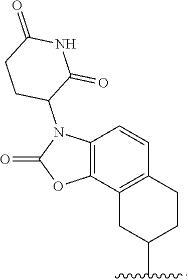 [SMILES: *C1CCc2ccc3c(oc(=O)n3C3CCC(=O)NC3=O)c2C1]

In some embodiments, Z is selected from the group consisting of:

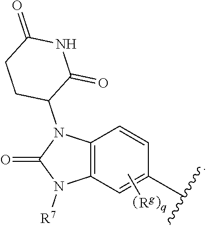

In some embodiments, Z is selected from the group consisting of:

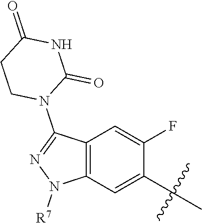

In some embodiments, Z is selected from the group consisting of:

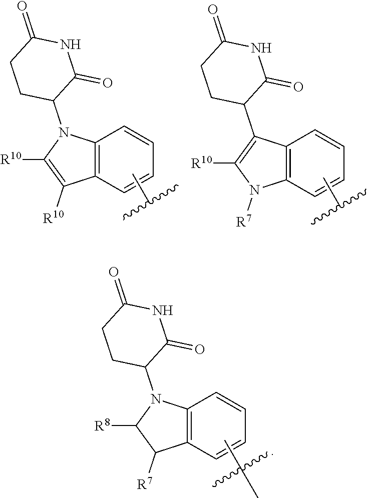

In some embodiments, Z is

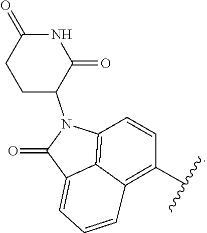 [SMILES: *c1ccc2c3c(cccc13)C(=O)N2C1CCC(=O)NC1=O]

In some embodiments, Z is

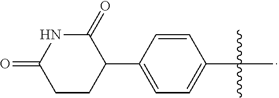 [SMILES: *c1ccc(C2CCC(=O)NC2=O)cc1]

In some embodiments, Z is

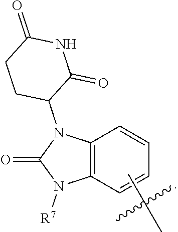

In some embodiments, Z is

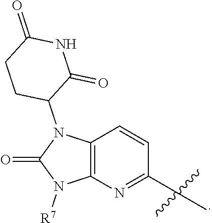

In some embodiments, Z is

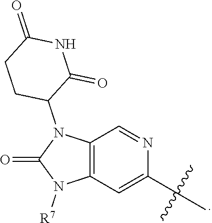

In some embodiments, Z is

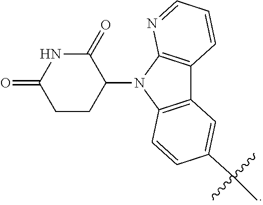 [SMILES: *c1ccc2c(c1)c1cccnc1n2C1CCC(=O)NC1=O]

In some embodiments, R⁷, if present, is hydrogen. In some embodiments, R⁷, if present, is C1-C6 alkyl. In some embodiments, R⁷, if present, is C1-C3 alkyl. In some embodiments, R⁷, if present, is methyl. In some embodiments, R⁷, if present, is C1-C6 alkyl substituted with one group selected from hydroxyl, cyano and C1-C6 alkoxy. In some embodiments, R⁷, if present, is C1-C6 haloalkyl. In some embodiments, R⁷, if present, is C3-C6 cycloalkyl, or 4-6 membered heterocyclyl, —(CR^{A}R^{B})(4-12 membered heterocyclyl), or —(CR^{A}R^{B})(C3-C6 cycloalkyl). The In some embodiments, each R^{A} and R^{B} are hydrogen.

In some embodiments, R⁸, if present, is hydrogen. In some embodiments, R⁸, if present, is C1-C6 alkyl. In some embodiments, R⁸, if present, is C1-C3 alkyl.

In some embodiments, q is 0 or 1. In some embodiments, q is 0. In some embodiments, q is 1.

In some embodiments, R⁹, if present, is hydrogen. In some embodiments, R⁹, if present, is halogen. In some embodiments, R⁹, if present, is cyano. In some embodiments, R⁹, if present, is C1-C6 alkyl or C1-C6 haloalkyl. In some embodiments, R⁹, if present, is C1-C6 alkoxy, C1-C5 cycloalkoxy, 5-10 membered heteroaryloxy, or phenoxy.

In some embodiments, each R¹⁰, when present, is hydrogen. In some embodiments, one R¹⁰ is cyano, and the remaining R¹⁰, if present, are hydrogen. In some embodiments, one R¹⁰ is halogen, and the remaining R¹⁰, if present, are hydrogen. In some embodiments, the halogen is fluoro. In some embodiments, one R¹⁰ is C1-C6 alkyl, C1-C6 haloalkyl, or C3-C6 cycloalkyl, and the remaining R¹⁰, if present, are hydrogen.

In some embodiments, the compound of Formula (I) is a compound of Formula (I-a):

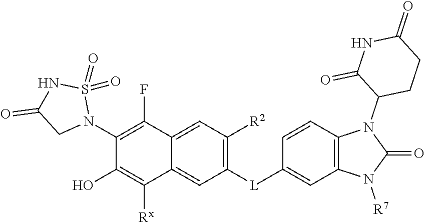

or a pharmaceutically acceptable salt thereof.

In some embodiments, the compound of Formula (I) is a compound of Formula (I-b):

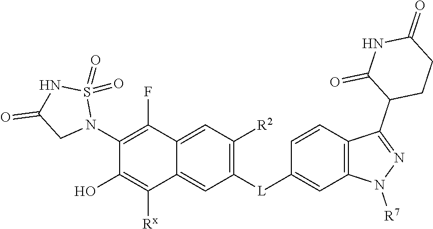

or a pharmaceutically acceptable salt thereof.

In some embodiments, the compound of Formula (I) is a compound of Formula (I-c):

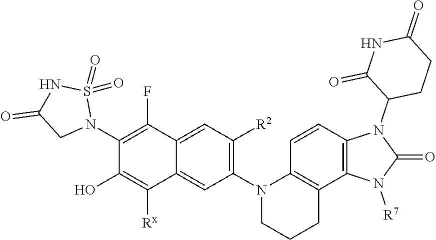

or a pharmaceutically acceptable salt thereof.

In some embodiments, the compound of Formula (I) is a compound of Formula (I-d):

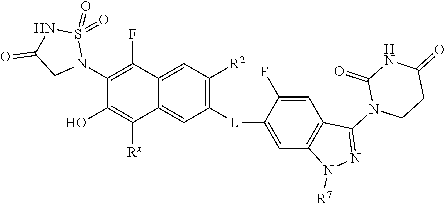

or a pharmaceutically acceptable salt thereof.

In some embodiments of a compound of Formula (I), R⁷ is C1-C3 alkyl. In some embodiments of a compound of Formula (I), R⁷ is methyl, ethyl, or isopropyl. In some embodiments of a compound of Formula (I), R⁷ is methyl. In some embodiments of a compound of Formula (I), R⁷ is —(CH₂)₂OCH₃. In some embodiments of a compound of Formula

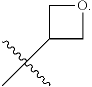 [SMILES: *C1COC1]

In some embodiments of a compound of Formula (I), R⁷ is hydrogen.

In some embodiments, the compound of Formula (I) is a compound of Formula (I-e):

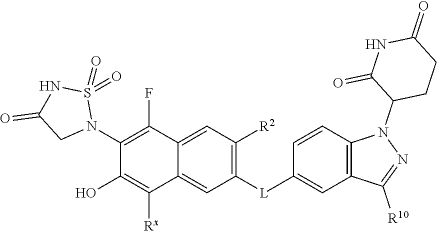

or a pharmaceutically acceptable salt thereof.

In some embodiments, R¹⁰ is methyl.

In some embodiments, the compound of Formula (I) is a compound of Formula (I-f):

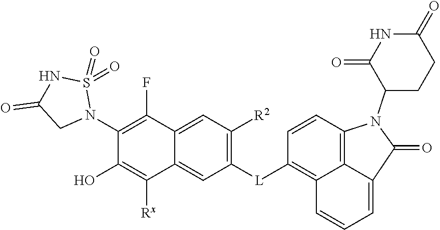

or a pharmaceutically acceptable salt thereof.

In some embodiments of a compound of Formula (I), R² is halogen. In some embodiments of a compound of Formula (I), R² is C1-C3 alkoxy, C3-C6 cycloalkoxy, C1-C3 haloalkoxy, C1-C3 haloalkyl, or C3-C5 halocycloalkoxy. In some embodiments of a compound of Formula (I), R² is C1-C3 alkyl or C3-C6 cycloalkyl. In some embodiments, the compound of Formula (I) is a compound of Formula (II-a):

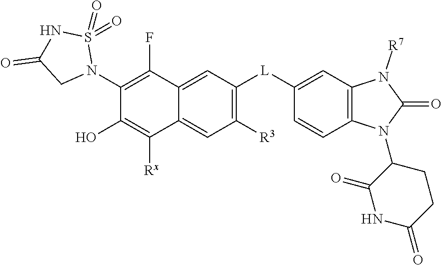

or a pharmaceutically acceptable salt thereof.

In some embodiments, the compound of Formula (I) is a compound of Formula (II-b):

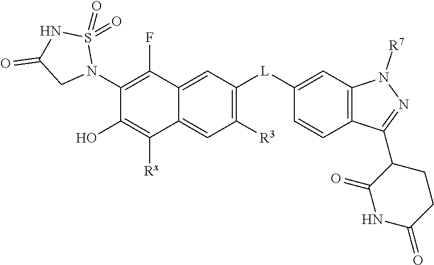

or a pharmaceutically acceptable salt thereof.

In some embodiments, the compound of Formula (I) is a compound of Formula (II-c):

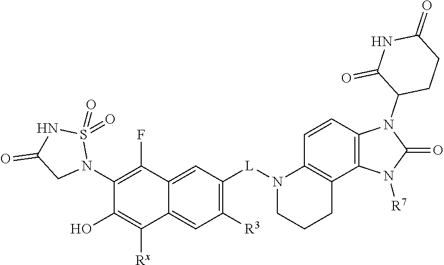

or a pharmaceutically acceptable salt thereof.

In some embodiments, the compound of Formula (I) is a compound of Formula (II-d):

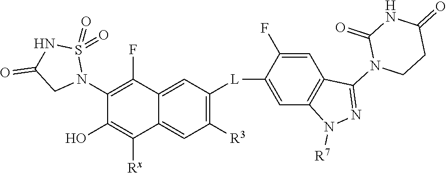

or a pharmaceutically acceptable salt thereof. In some embodiments of a compound of Formula (I), R⁷ is C1-C3 alkyl. In some embodiments of a compound of Formula (I), R⁷ is methyl, ethyl, or isopropyl. In some embodiments of a compound of Formula (I), R⁷ is methyl. In some embodiments of a compound of Formula (I), R⁷ is —(CH₂)₂OCH₃. In some embodiments of compound of Formula (I), R⁷ is

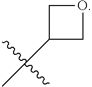 [SMILES: *C1COC1]

In some embodiments of a compound of Formula (I), R⁷ is hydrogen.

In some embodiments, the compound of Formula (I) is a compound of Formula (II-e):

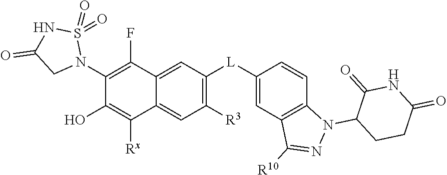

or a pharmaceutically acceptable salt thereof.

In some embodiments of Formula (I), R¹⁰ is methyl.

In some embodiments, the compound of Formula (I) is a compound of Formula (II-f):

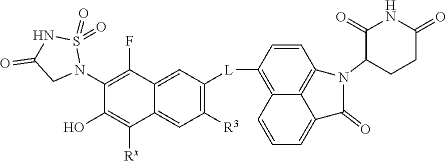

or a pharmaceutically acceptable salt thereof.

In some embodiments of a compound of Formula (I), R³ is hydrogen. In some embodiments of a compound of Formula (I), R³ is halogen. In some embodiments of a compound of Formula (I), R³ is C1-C3 alkoxy, C3-C6 cycloalkoxy, C1-C3 haloalkoxy, or C3-C5 halocycloalkoxy. In some embodiments of a compound of Formula (I), R³ is C1-C3 alkyl or C3-C6 cycloalkyl. In some embodiments of a compound of Formula (I), R^{x} is hydrogen. In some embodiments of a compound of Formula (I), R^{x} is halogen.

In some embodiments of compounds of Formula (I-a) to Formula (II-f), L is —U—V—W—X—Y—.

In some embodiments of compounds of Formula (I-a) to Formula (II-f), U is —NR⁴(C═O)— or —(C═O)NR⁴—, or —(NR⁴)(C═O)(NR⁴)—; V is a bond, C1-C6 alkylene, or C3-C6 cycloalkylene; W is a bond; and X is a bond, C6-C10 arylene, or C1-C3 alky. In some embodiments of compounds of Formula (I-a) to Formula (II-f), U is —NR⁴(C═O)— or —(C═O)NR⁴—, or —(NR⁴)(C═O)(NR⁴)—; V is a bond or C1-C6 alkylene, 4-10 membered heterocyclyene, or C3-C6 cycloalkylene; W is a bond; and X is 4-12-membered heterocyclylene or a bond. In some embodiments of compounds of Formula (I-a) to Formula (II-f), U is —NR⁴(C═O)—. In some embodiments of compounds of Formula (I-a) to Formula (II-f), U is —(C═O)NR⁴—. In some embodiments of compounds of Formula (I-a) to Formula (II-f), U is —(NR⁴)(C═O)(NR⁴)—. In some embodiments of compounds of Formula (I-a) to Formula (II-f), V is a bond. In some embodiments of compounds of Formula (I-a) to Formula (II-f), V is C1-C3 alkylene. In some embodiments of compounds of Formula (I-a) to Formula (II-f), V is methylene or ethylene. In some embodiments of compounds of Formula (I-a) to Formula (II-f), V is W is a bond; and X is a bond.

In some embodiments of compounds of Formula (I-a) to Formula (II-f), U is —NR⁴(C═O)—, —(C═O)NR⁴—, or —(NR⁴)(C═O)(NR⁴)—; V is a bond, C1-C6 alkylene, 4-10 membered heterocyclyene, or C3-C6 cycloalkylene; W is 4-10 membered heterocyclyene, C3-C6 cycloalkylene, C1-C3 alkylene optionally substituted with hydroxyl, —(NR⁴)R⁵—, —(NR⁴)(C═O)—, or —O—; and X is a bond, C6-C10 arylene, or R⁶ is C1-C3 alkylene.

In some embodiments of compounds of Formula (I-a) to Formula (II-f), U is —O—; V is C1-C6 alkylene, C3-C6 cycloalkylene, or 4-10-membered heterocyclylene; W is a bond, —C(═O)—, —C(═O)R⁵—, —R⁵(C═O)—, —N(R⁴)—, —C(═O)NR⁴—, —NR⁴C(═O)—, —NR⁴C(═O)R⁵—, or —(S═O)—, or —S(O₂)—. In some embodiments of compounds of Formula (I-a) to Formula (II-f), V is C1-C6 alkylene. In some embodiments of compounds of Formula (I-a) to Formula (II-f), V is C1-C3 alkylene. In some embodiments of compounds of Formula (I-a) to Formula (II-f), V is methylene or ethylene. In some embodiments of compounds of Formula (I-a) to Formula (II-f), V is 4-10-membered heterocyclylene. In some embodiments of compounds of Formula (I-a) to Formula (II-f), W is —C(═O)—, —C(═O)R⁵—, a bond, or —C(═O)NR⁴—. In some embodiments of compounds of Formula (I-a) to Formula (II-f), W is —NR⁴C(═O)—. In some embodiments of compounds of Formula (I-a) to Formula (II-f), W is —NR⁴C(═O)R⁵—. In some embodiments of compounds of Formula (I-a) to Formula (II-f), W is —C(═O)R⁵—. In some embodiments of compounds of Formula (I-a) to Formula (II-f), W is a bond. In some embodiments of compounds of Formula (I-a) to Formula (II-f), R⁴ is hydrogen. In some embodiments of compounds of Formula (I-a) to Formula (II-f), R⁵ is C1-C3 alkylene. In some embodiments of compounds of Formula (I-a) to Formula (II-f), R⁵ is —CH₂—. In some embodiments of compounds of Formula (I-a) to Formula (II-f), R⁵ is C3-C7 cycloalkylene.

In some embodiments of compounds of Formula (I-a) to Formula (II-f), U is —NR⁴—; V is C1-C6 alkylene or a bond; W is —C(═O)— or —C(═O)R⁵—; and X is a bond. In some embodiments of compounds of Formula (I-a) to Formula (II-f), U is —NH—. In some embodiments of compounds of Formula (I-a) to Formula (II-f), U is —N(C1-C3 alkyl)-. In some embodiments of compounds of Formula (I-a) to Formula (II-f), V is C1-C3 alkylene. In some embodiments of compounds of Formula (I-a) to Formula (II-f), V is methylene or ethylene. In some embodiments of compounds of Formula (I-a) to Formula (II-f), W is —C(═O)—. In some embodiments of compounds of Formula (I-a) to Formula (II-f), W is —C(═O)R⁵—. In some embodiments of compounds of Formula (I-a) to Formula (II-f), R⁵ is C1-C3 alkylene.

In some embodiments of compounds of Formula (I-a) to Formula (II-f), U is a bond, C1-C3 alkylene, C2-C3 alkenylene, 4-10 membered heterocyclylene, or C2-C3 alkynylene; V is a bond or 4-10 membered heterocyclylene; W is a bond or C(═O or —C(═O)R⁵—; and X is a bond, C1-C3 alkylene, or C6-C10 arylene. In some embodiments of compounds of Formula (I-a) to Formula (II-f), U is a bond. In some embodiments of compounds of Formula (I-a) to Formula (II-f), U is C2-C3 alkenylene. In some embodiments of compounds of Formula (I-a) to Formula (II-f), U is 4-10 membered heterocyclylene. In some embodiments of compounds of Formula (I-a) to Formula (II-f), U is C2-C3 alkynylene. In some embodiments of compounds of Formula (I-a) to Formula (II-f), W is a bond. In some embodiments of compounds of Formula (I-a) to Formula (II-f), W is C(═O). In some embodiments of compounds of Formula (I-a) to Formula (II-f), W is C(═O)R⁵—. In some embodiments of compounds of Formula (I-a) to Formula (II-f), X is a bond. In some embodiments of compounds of Formula (I-a) to Formula (II-f), X is C1-C3 alkylene. In some embodiments of compounds of Formula (I-a) to Formula (II-f), X is C6-C10 arylene.

In some embodiments of Formula (I-a) to Formula (II-f), U is —NR⁴(C═O)—, —(C═O)NR⁴—, or —(NR⁴)(C═O)(NR⁴)—; V is C1-C6 alkylene; W is a bond; and X is a bond. In some embodiments of Formula (I-a) to Formula (II-f), U is —NR⁴(C═O)—. In some embodiments of Formula (I-a) to Formula (II-f), U is —(C═O)NR⁴—. In some embodiments of Formula (I-a) to Formula (II-f), U is —(NR⁴)(C═O)(NR⁴)—. In some embodiments of Formula (I-a) to Formula (II-f), V is C1-C6 alkylene. In some embodiments of Formula (I-a) to Formula (II-f), W is a bond and X is a bond. In some embodiments of Formula (I-a) to Formula (II-f), each R⁴ within U is hydrogen.

In some embodiments of compounds of Formula (I-a) to Formula (II-f), Y is R⁶. In some embodiments of compounds of Formula (I-a) to Formula (II-f), R⁶ is 4-12 membered heterocyclylene optionally substituted with 1-3 substituents independently selected from fluoro, hydroxyl, C1-C6 alkoxy, and C1-C6 alkyl. In some embodiments of compounds of Formula (I-a) to Formula (II-f), R⁶ is 4-8 membered heterocyclylene optionally substituted with 1-3 substituents independently selected from fluoro, hydroxyl, C1-C6 alkoxy, and C1-C6 alkyl. In some embodiments of compounds of Formula (I-a) to Formula (II-f), R⁶ is selected from the group consisting of:

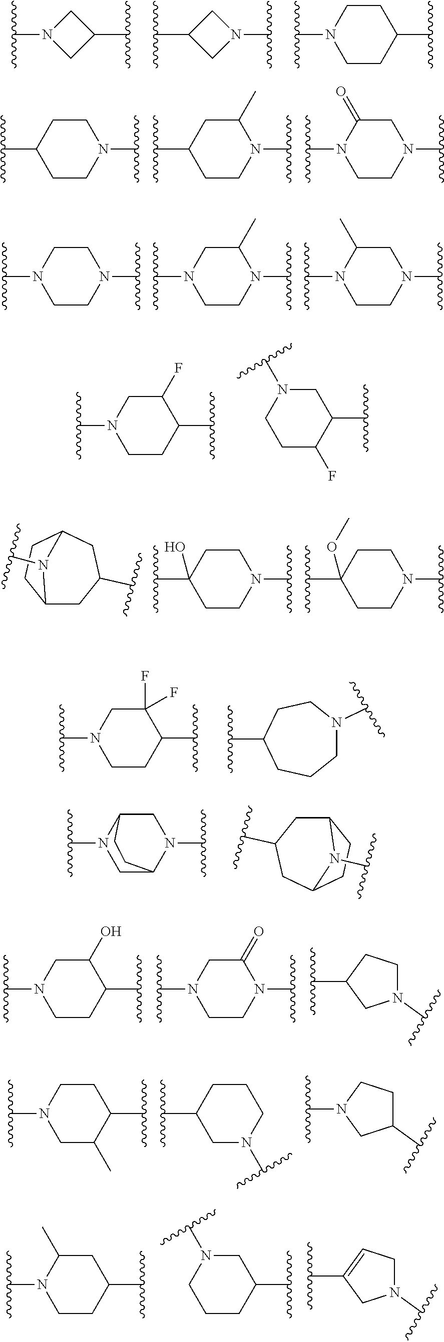

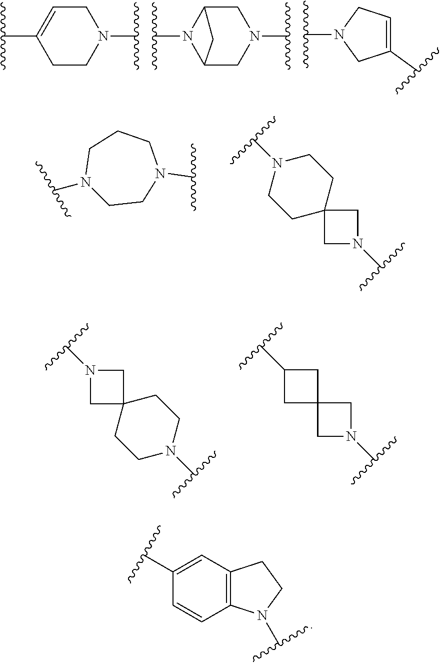

In some embodiments of compounds of Formula (I-a) to Formula (II-f), R⁶ is

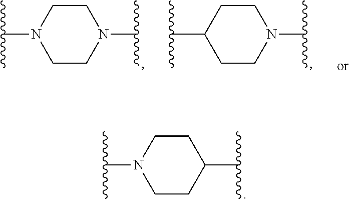

In some embodiments of compounds of Formula (I-a) to Formula (I-f), R⁶ is

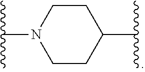 [SMILES: *C1CCN(*)CC1]

In some embodiments of compounds of Formula (I-a) to Formula (II-f), R⁶ is 7-12 membered bicyclic heterocyclylene. In some embodiments of compounds of Formula (I-a) to Formula (II-f), R⁶ is 4-8 membered heterocyclylene substituted with methyl, hydroxyl, methoxy, oxo, or 1 or 2 fluoros. In some embodiments of compounds of Formula (I-a) to Formula (II-f), R⁶ is 7-12 membered bicyclic spirocyclic heterocyclylene. In some embodiments of compounds of Formula (I-a) to Formula (II-f), R⁶ is

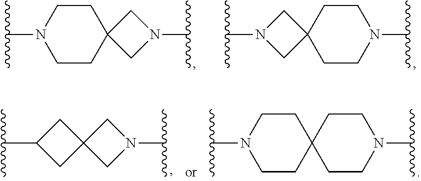

In some embodiments of compounds of Formula (I-a) to Formula (II-f), R⁶ is C1-C3 alkylene.

In some embodiments of Formula (I-a) to Formula (II-f), R⁶ is 5-10 membered heteroarylene. In some embodiments of Formula (I-a) to Formula (II-f), R⁶ is 5-6 membered heteroarylene. In some embodiments of Formula (I-a) to Formula (II-f), R⁶ is selected from the group consisting of:

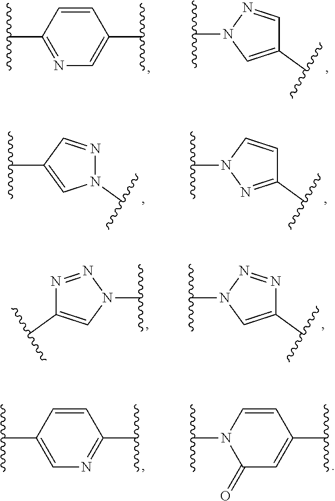

In some embodiments of compounds of Formula (I-a) to Formula (II-f), R⁶ is C1-C3 alkylene.

In some embodiments of compounds of Formula (I-a) to Formula (II-f), Y is —R⁶(CR^{A}R^{B})ₚ-Q-; and p is 0. In some embodiments of compounds of Formula (I-a) to Formula (II-f), Y is —R⁶NR⁴— or —R⁶O—. In some embodiments of compounds of Formula (I-a) to Formula (II-f), Y is —R⁶NH. In some embodiments of compounds of Formula (I-a) to Formula (II-f), Y is —R⁶O—. In some embodiments of compounds of Formula (I-a) to Formula (II-f), Y is R⁶(CR^{A}R^{B})ₚ-Q- or -Q-(CR^{A}R^{B})ₚR⁶—; p is 1 or 2; and each R^{A} and R^{B} are hydrogen. In some embodiments of compounds of Formula (I-a) to Formula (II-f), Y is —R⁶CH₂—O— or —R⁶CH₂—N(R⁴)—. In some embodiments of compounds of Formula (I-a) to Formula (II-f), Y is —R⁶CH₂—O—. In some embodiments of compounds of Formula (I-a) to Formula (II-f), Y is —R⁶CH₂—NH. In some embodiments of compounds of Formula (I-a) to Formula (II-f), Y is —R⁶(CR^{A}R^{B})ₚ-Q- or -Q-(CR^{A}R^{B})ₚR⁶—; p is 1 or 2; and each R^{A} and R^{B} are independently hydrogen or C1-C3 alkyl; or one pair of R^{A} and R^{B}, together with the carbon atom to which they are attached, come together to form a C3-C4 cycloalkyl, and each remaining R^{A} and R^{B}, if present, are hydrogen.

In some embodiments of compounds of Formula (I-a) to Formula (II-f), the —(CR^{A}R^{B})ₚ-Q- portion of Y is selected from the group consisting of:

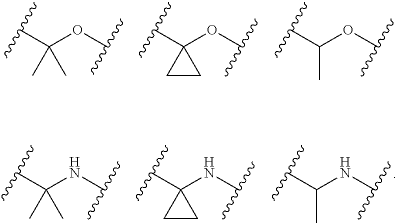

In some embodiments of compounds of Formula (I-a) to Formula (II-f), Y is —R⁶C(═O)(CR^{A}R^{B})-Q-; and each R^{A} and R^{B} are independently hydrogen, fluoro, or C—C3 alkyl.

In some embodiments of compounds of Formula (I-a) to Formula (II-f), the —(CR^{A}R^{B})ₚ-Q- portion of Y is selected from the group consisting of:

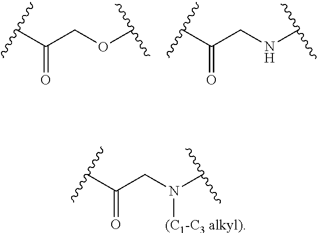

In some embodiments of compounds of Formula (I-a) to Formula (II-f), R⁶ is 4-12 membered heterocyclylene optionally substituted with 1-3 substituents independently selected from fluoro, hydroxyl, C1-C6 alkoxy, and C1-C6 alkyl. In some embodiments of compounds of Formula (I-a) to Formula (II-f), R⁶ is 4-8 membered heterocyclylene optionally substituted with 1-3 substituents independently selected from fluoro, hydroxyl, C1-C6 alkoxy, and C1-C6 alkyl. In some embodiments of compounds of Formula (I-a) to Formula (II-f), R⁶ is selected from the group consisting of:

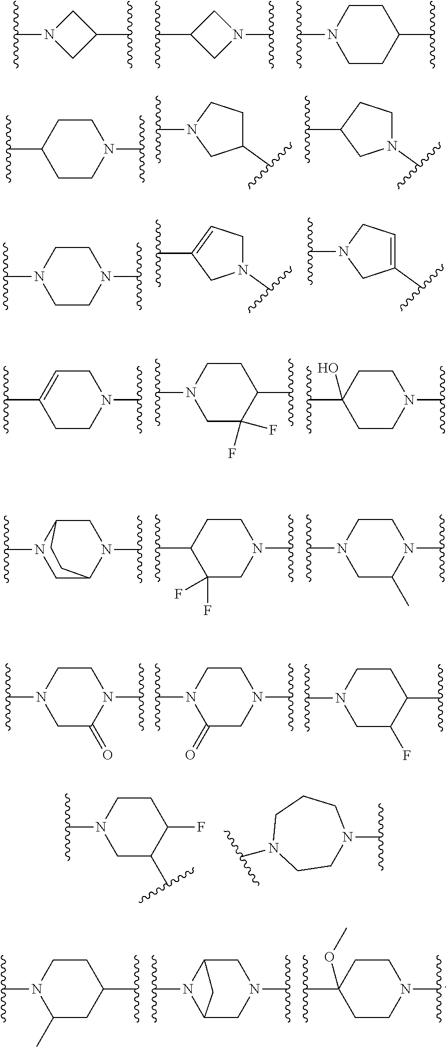

In some embodiments of compounds of Formula (I-a) to Formula (II-f), R⁶ is

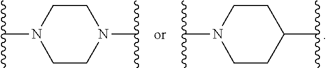 [SMILES: *N1CCN(*)CC1 ; *C1CCN(*)CC1]

In some embodiments of compounds of Formula (I-a) to Formula (II-f), R⁶ is

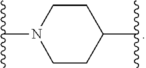 [SMILES: *C1CCN(*)CC1]

In some embodiments of compounds of Formula (I-a) to Formula (II-f), R⁶ is 7-12 membered bicyclic heterocyclylene. In some embodiments of compounds of Formula (I-a) to Formula (II-f), R⁶ is 7-12 membered bicyclic spirocyclic heterocyclylene. In some embodiments of compounds of Formula (I-a) to Formula (II-f), R⁶ is

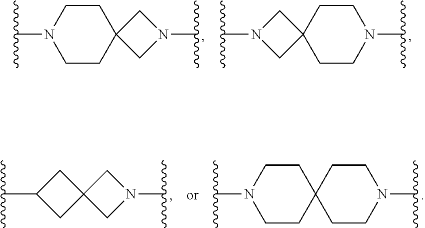

In some embodiments of compounds of Formula (I-a) to Formula (II-f), R⁶ is C6-C10 arylene. In some embodiments of compounds of Formula (I-a) to Formula (II-f), R⁶ is phenylene.

In some embodiments of compounds of Formula (I-a) to Formula (II-f), R⁶ is 5-10 membered heteroarylene. In some embodiments of compounds of Formula (I-a) to Formula (II-f), R⁶ is 5-6 membered heteroarylene. In some embodiments of compounds of Formula (I-a) to Formula (II-f), R⁶ is 5-6 membered heteroarylene. In some embodiments of compounds of Formula (I-a) to Formula (II-f), R⁶ is triazolylene, pyrazolylene, or pyridinylene. In some embodiments of compounds of Formula (I-a) to Formula (II-f), R⁶ is selected from the group consisting of:

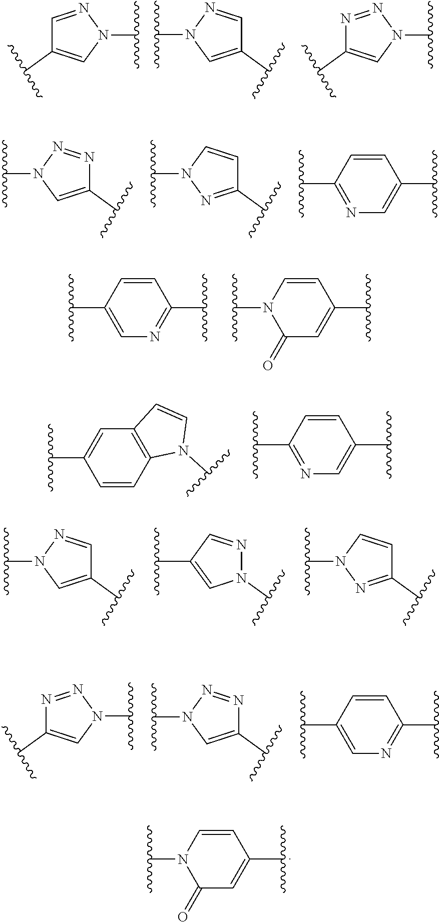

In some embodiments, R¹ is fluoro; R^{x} is hydrogen; R² is hydrogen; R³ is -L-Z;
Z is

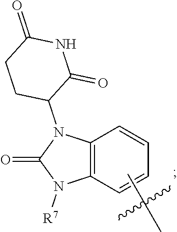

and R⁷ is hydrogen or C1-C6 alkyl.

In some embodiments, R¹ is fluoro; R^{x} is hydrogen; R² is -L-Z; R³ is hydrogen;
Z is

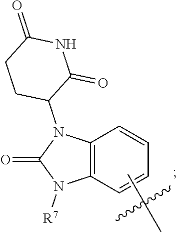

and R⁷ is hydrogen or C1-C6 alkyl.

In some embodiments:
U is —(NR⁴)C═O)—, —(C═O)NR⁴—, or —(NR⁴)(C═O)(NR⁴)—;
V is a bond, C1-C6 alkylene, or 4-6-membered heterocyclylene optionally substituted with methyl, hydroxyl, methoxy, or 1 or 2 fluoros;
W is a bond or C1-C3 alkylene;
X is a bond or C1-C3 alkylene;
Y is R⁶;
R⁶ is C3-C7 cycloalkylene, 4-12 membered heterocyclylene, C6-C10 arylene, or 5-10 membered heteroarylene; and
R⁴ is hydrogen or C1-C6 alkyl.

In some embodiments:
U is —(NR⁴)C═O)—, —(C═O)NR⁴—, or —(NR⁴)(C═O)(NR⁴)—;
V is a bond or 4-6-membered heterocyclylene optionally substituted with methyl, hydroxyl, methoxy, or 1 or 2 fluoros;
W is a bond or C1-C3 alkylene;
X is a bond or C1-C3 alkylene;
Y is R⁶;
R⁶ is 4-8 membered heterocyclylene, phenyl, or 5-6 membered heteroarylene; and
R⁴ is hydrogen or C1-C6 alkyl.

In some embodiments, V and X are bonds. In some embodiments, R⁶ is piperidinyl, piperazinyl, phenyl, pyridinyl, or pyridonyl. In some embodiments, W is C1-C3 alkylene and R⁴ is hydrogen.

In some embodiments, U is —(NR⁴)C═O)—, V is a bond, W is C1-C3 alkylene, X is a bond, and Y is R⁶. In some embodiments, R⁴ is hydrogen or methyl; and R⁶ is 5-6 membered heterocyclylene, phenyl, or 5-6 membered heteroarylene. In some embodiments, R⁶ is piperidinyl, piperazinyl, phenyl, pyridinyl, or pyridonyl. In some embodiments, R¹ is fluoro; RN is hydrogen; R² is hydrogen; R³ is -L-Z;
Z is

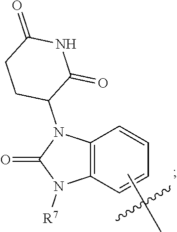

R⁷ is hydrogen or C1-C6 alkyl; L is —U—V—W—X—Y—; U is —(NH)C═O)—, —(C═O)NH—, or —(NH)(C═O)(NH)—; V is a bond; W is methylene or ethylene; X is a bond; Y is R⁶; and R⁶ is piperidinyl, piperazinyl, phenyl, pyridinyl, or pyridonyl.

In some embodiments, R¹ is fluoro; RN is hydrogen; R² is -L-Z; R³ is hydrogen;
Z is

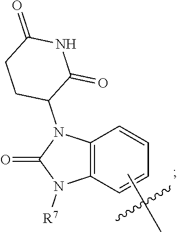

R⁷ is hydrogen or C1-C6 alkyl; L is —U—V—W—X—Y—; U is —(NH)C═O)—, —(C═O)NH—, or —(NH)(C═O)(NH)—; V is a bond; W is methylene or ethylene; X is a bond; Y is R⁶; and R⁶ is piperidinyl, piperazinyl, phenyl, pyridinyl, or pyridonyl.

In some embodiments, one of U, V, W, and X is a bond. In some embodiments, two of U, V, W, and X is a bond. In some embodiments, three of U, V, W, and X is a bond. In some embodiments, U, V, W, and X cannot each be a bond.

In some embodiments, a compound of Formula (I) is selected from a compound set forth in Table 1, or a pharmaceutically acceptable salt thereof.

TABLE 1
Exemplary compounds of the disclosure.

| Degrader | Compound |
|---|---|
| No. | |
| 1 | 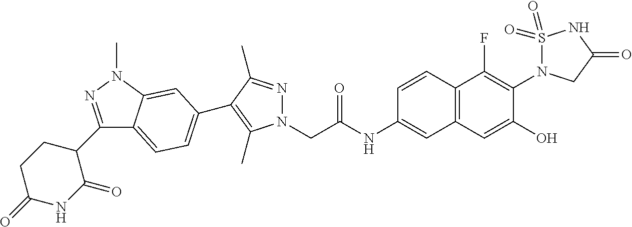 [SMILES: Cc1nn(CC(=O)Nc2ccc3c(F)c(N4CC(=O)NS4(=O)=O)c(O)cc3c2)c(C)c1-c1ccc2c(C3CCC(=O)NC3=O)nn(C)c2c1] |
| 2 | 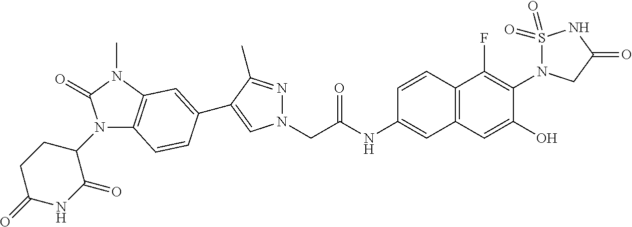 [SMILES: Cc1nn(CC(=O)Nc2ccc3c(F)c(N4CC(=O)NS4(=O)=O)c(O)cc3c2)cc1-c1ccc2c(c1)n(C)c(=O)n2C1CCC(=O)NC1=O] |
| 3 | 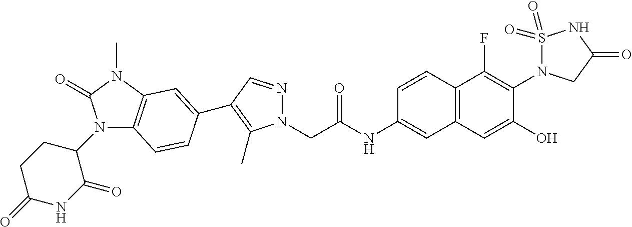 [SMILES: Cc1c(-c2ccc3c(c2)n(C)c(=O)n3C2CCC(=O)NC2=O)cnn1CC(=O)Nc1ccc2c(F)c(N3CC(=O)NS3(=O)=O)c(O)cc2c1] |
| 4 | 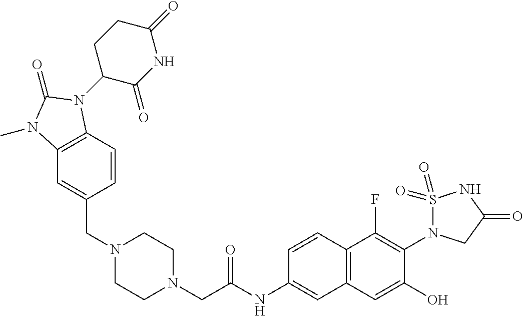 [SMILES: Cn1c(=O)n(C2CCC(=O)NC2=O)c2ccc(CN3CCN(CC(=O)Nc4ccc5c(F)c(N6CC(=O)NS6(=O)=O)c(O)cc5c4)CC3)cc21] |
| 5 | 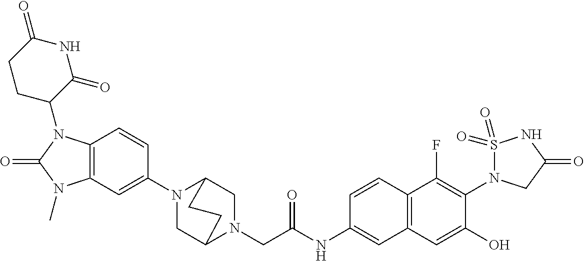 [SMILES: Cn1c(=O)n(C2CCC(=O)NC2=O)c2ccc(N3CC4CCC3CN4CC(=O)Nc3ccc4c(F)c(N5CC(=O)NS5(=O)=O)c(O)cc4c3)cc21] |
| 6 | 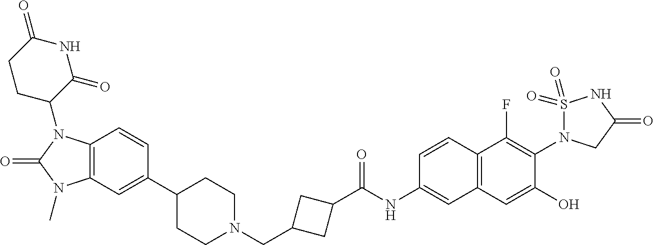 [SMILES: Cn1c(=O)n(C2CCC(=O)NC2=O)c2ccc(C3CCN(CC4CC(C(=O)Nc5ccc6c(F)c(N7CC(=O)NS7(=O)=O)c(O)cc6c5)C4)CC3)cc21] |
| 7 | 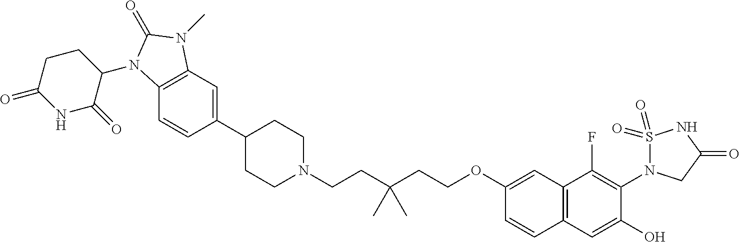 [SMILES: Cn1c(=O)n(C2CCC(=O)NC2=O)c2ccc(C3CCN(CCC(C)(C)CCOc4ccc5cc(O)c(N6CC(=O)NS6(=O)=O)c(F)c5c4)CC3)cc21] |
| 8 | 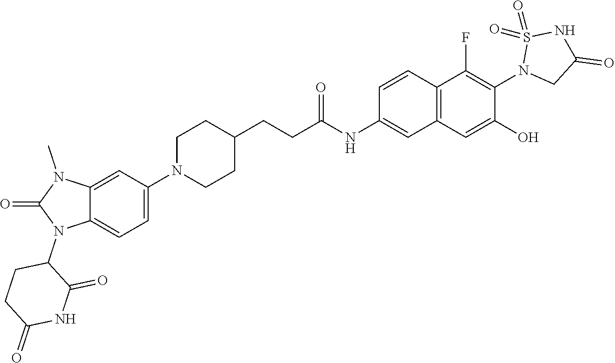 [SMILES: Cn1c(=O)n(C2CCC(=O)NC2=O)c2ccc(N3CCC(CCC(=O)Nc4ccc5c(F)c(N6CC(=O)NS6(=O)=O)c(O)cc5c4)CC3)cc21] |
| 9 | 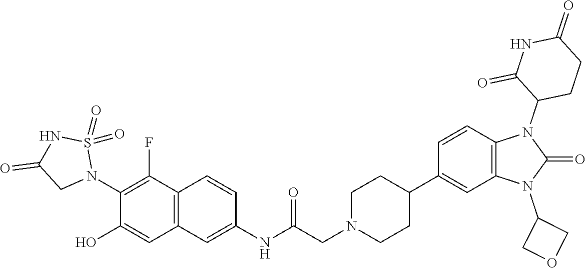 [SMILES: O=C1CCC(n2c(=O)n(C3COC3)c3cc(C4CCN(CC(=O)Nc5ccc6c(F)c(N7CC(=O)NS7(=O)=O)c(O)cc6c5)CC4)ccc32)C(=O)N1] |
| 10 | 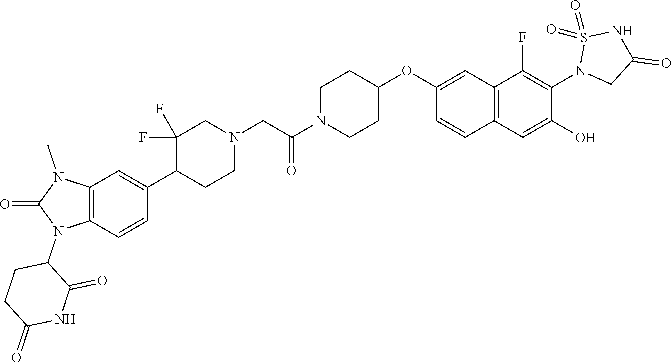 [SMILES: Cn1c(=O)n(C2CCC(=O)NC2=O)c2ccc(C3CCN(CC(=O)N4CCC(Oc5ccc6cc(O)c(N7CC(=O)NS7(=O)=O)c(F)c6c5)CC4)CC3(F)F)cc21] |
| 11 | 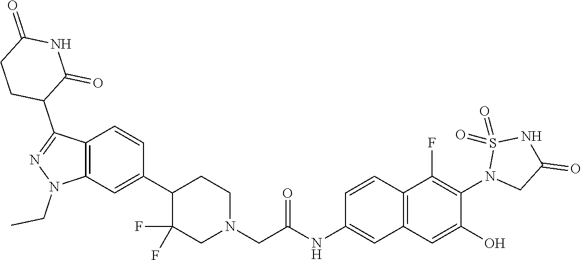 [SMILES: CCn1nc(C2CCC(=O)NC2=O)c2ccc(C3CCN(CC(=O)Nc4ccc5c(F)c(N6CC(=O)NS6(=O)=O)c(O)cc5c4)CC3(F)F)cc21] |
| 12 | 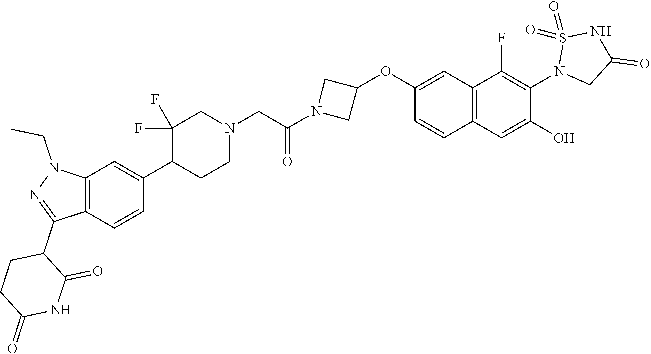 [SMILES: CCn1nc(C2CCC(=O)NC2=O)c2ccc(C3CCN(CC(=O)N4CC(Oc5ccc6cc(O)c(N7CC(=O)NS7(=O)=O)c(F)c6c5)C4)CC3(F)F)cc21] |
| 13 | 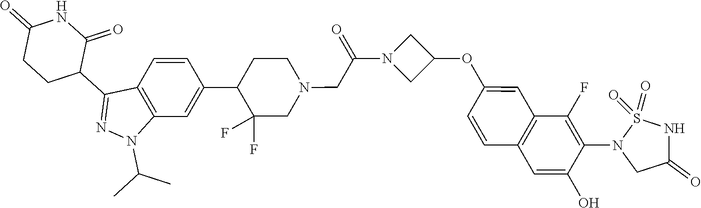 [SMILES: CC(C)n1nc(C2CCC(=O)NC2=O)c2ccc(C3CCN(CC(=O)N4CC(Oc5ccc6cc(O)c(N7CC(=O)NS7(=O)=O)c(F)c6c5)C4)CC3(F)F)cc21] |
| 14 | 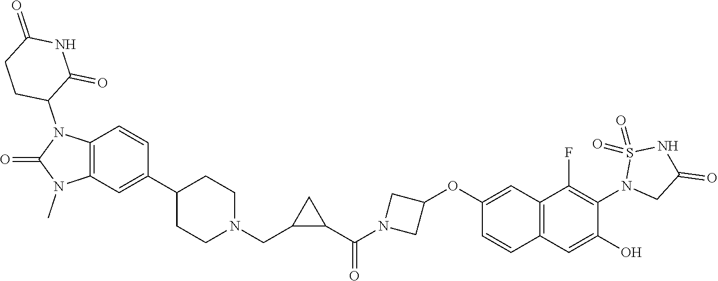 [SMILES: Cn1c(=O)n(C2CCC(=O)NC2=O)c2ccc(C3CCN(CC4CC4C(=O)N4CC(Oc5ccc6cc(O)c(N7CC(=O)NS7(=O)=O)c(F)c6c5)C4)CC3)cc21] |
| 15 | 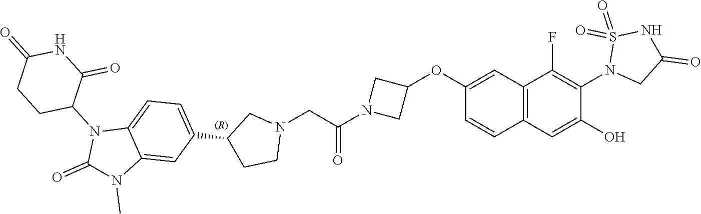 |
| 16 | 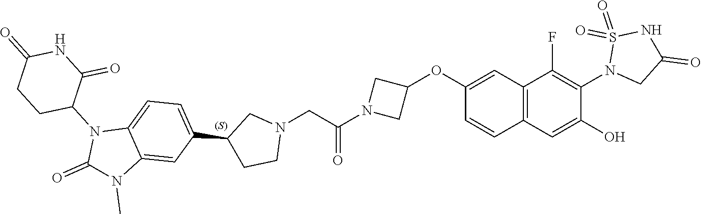 |
| 17 | 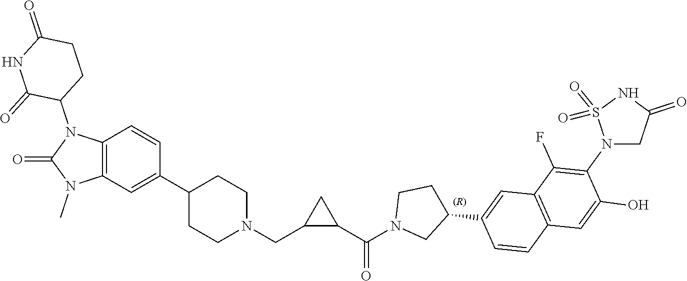 |
| 18 | 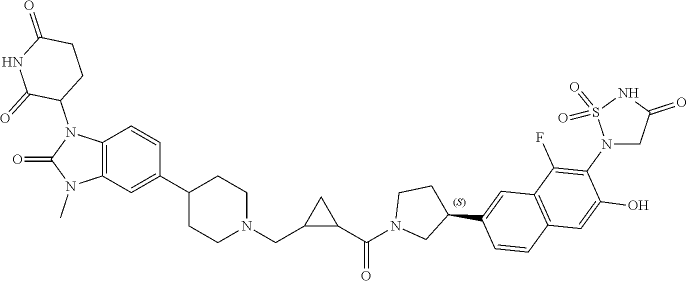 |
| 19 | 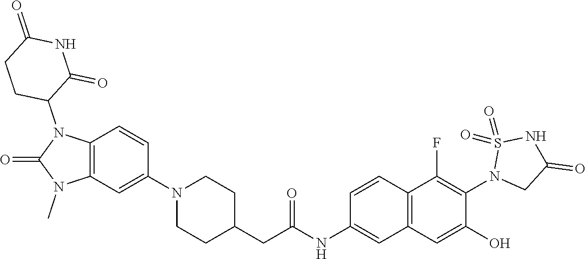 [SMILES: Cn1c(=O)n(C2CCC(=O)NC2=O)c2ccc(N3CCC(CC(=O)Nc4ccc5c(F)c(N6CC(=O)NS6(=O)=O)c(O)cc5c4)CC3)cc21] |
| 20 | 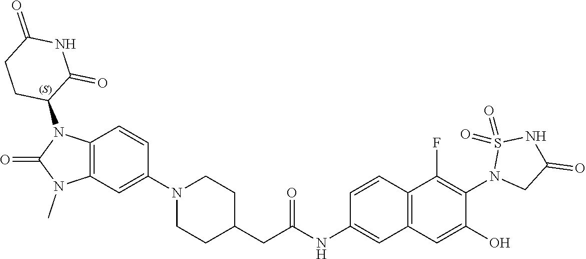 |
| 21 | 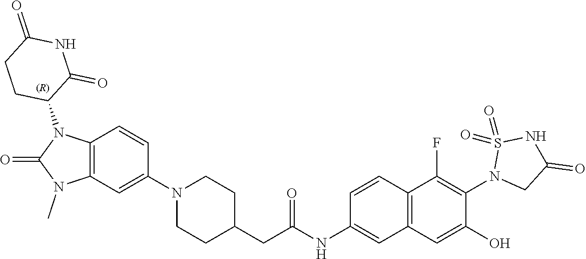 |
| 22 | 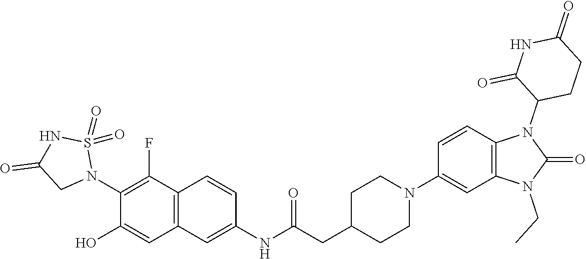 [SMILES: CCn1c(=O)n(C2CCC(=O)NC2=O)c2ccc(N3CCC(CC(=O)Nc4ccc5c(F)c(N6CC(=O)NS6(=O)=O)c(O)cc5c4)CC3)cc21] |
| 23 | 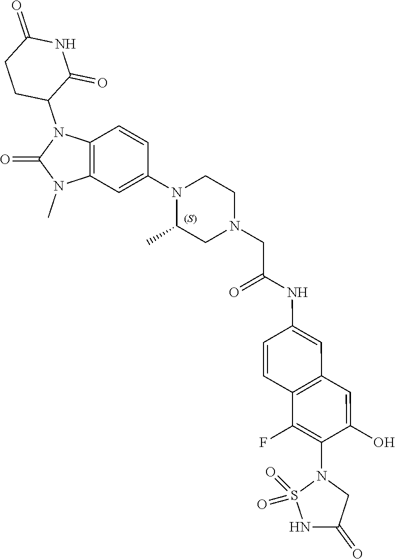 |
| 24 | 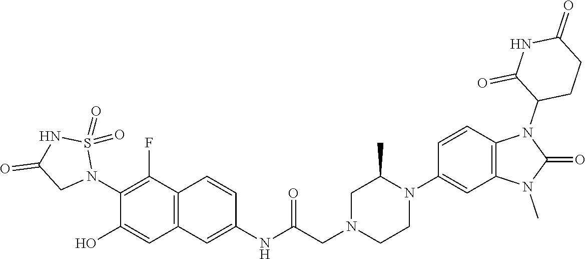 [SMILES: C[C@@H]1CN(CC(=O)Nc2ccc3c(F)c(N4CC(=O)NS4(=O)=O)c(O)cc3c2)CCN1c1ccc2c(c1)n(C)c(=O)n2C1CCC(=O)NC1=O] |
| 25 | 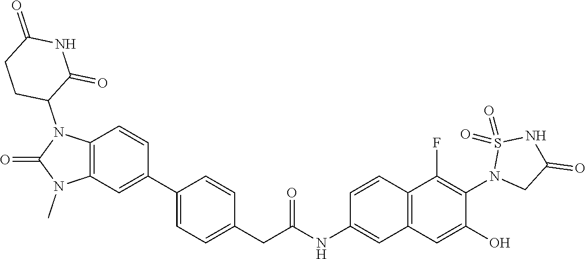 [SMILES: Cn1c(=O)n(C2CCC(=O)NC2=O)c2ccc(-c3ccc(CC(=O)Nc4ccc5c(F)c(N6CC(=O)NS6(=O)=O)c(O)cc5c4)cc3)cc21] |
| 26 | 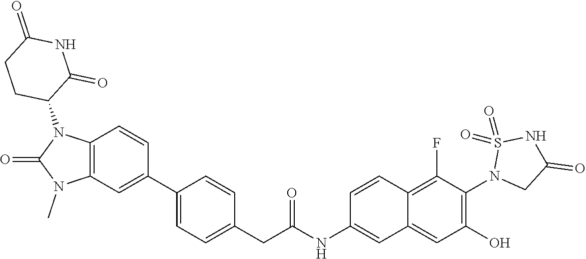 [SMILES: Cn1c(=O)n([C@@H]2CCC(=O)NC2=O)c2ccc(-c3ccc(CC(=O)Nc4ccc5c(F)c(N6CC(=O)NS6(=O)=O)c(O)cc5c4)cc3)cc21] |
| 27 | 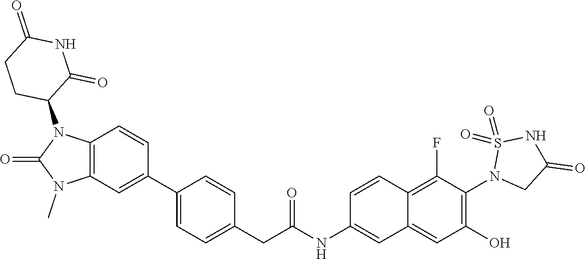 [SMILES: Cn1c(=O)n([C@H]2CCC(=O)NC2=O)c2ccc(-c3ccc(CC(=O)Nc4ccc5c(F)c(N6CC(=O)NS6(=O)=O)c(O)cc5c4)cc3)cc21] |
| 28 | 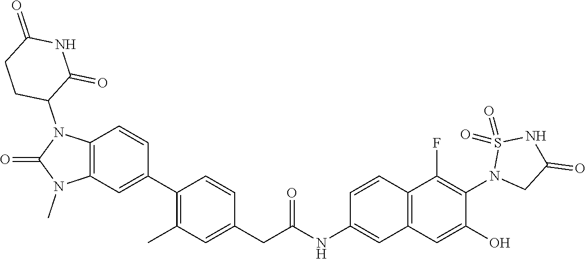 [SMILES: Cc1cc(CC(=O)Nc2ccc3c(F)c(N4CC(=O)NS4(=O)=O)c(O)cc3c2)ccc1-c1ccc2c(c1)n(C)c(=O)n2C1CCC(=O)NC1=O] |
| 29 | 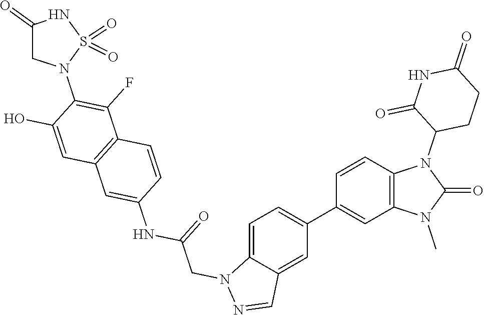 [SMILES: Cn1c(=O)n(C2CCC(=O)NC2=O)c2ccc(-c3ccc4c(cnn4CC(=O)Nc4ccc5c(F)c(N6CC(=O)NS6(=O)=O)c(O)cc5c4)c3)cc21] |
| 30 | 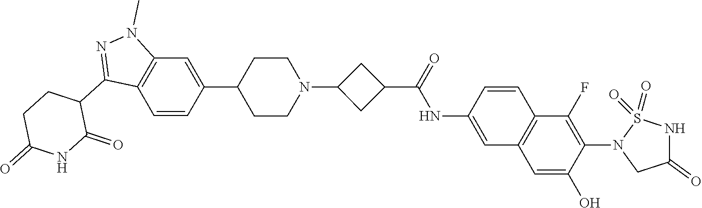 [SMILES: Cn1nc(C2CCC(=O)NC2=O)c2ccc(C3CCN(C4CC(C(=O)Nc5ccc6c(F)c(N7CC(=O)NS7(=O)=O)c(O)cc6c5)C4)CC3)cc21] |
| 31 | 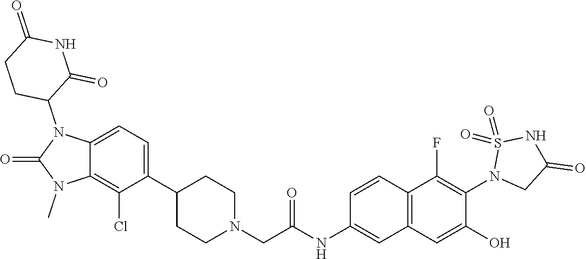 [SMILES: Cn1c(=O)n(C2CCC(=O)NC2=O)c2ccc(C3CCN(CC(=O)Nc4ccc5c(F)c(N6CC(=O)NS6(=O)=O)c(O)cc5c4)CC3)c(Cl)c21] |
| 32 | 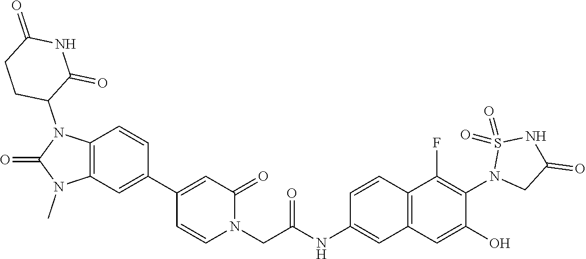 [SMILES: Cn1c(=O)n(C2CCC(=O)NC2=O)c2ccc(-c3ccn(CC(=O)Nc4ccc5c(F)c(N6CC(=O)NS6(=O)=O)c(O)cc5c4)c(=O)c3)cc21] |
| 33 | 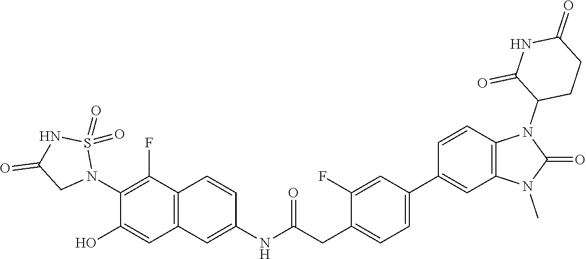 [SMILES: Cn1c(=O)n(C2CCC(=O)NC2=O)c2ccc(-c3ccc(CC(=O)Nc4ccc5c(F)c(N6CC(=O)NS6(=O)=O)c(O)cc5c4)c(F)c3)cc21] |
| 34 | 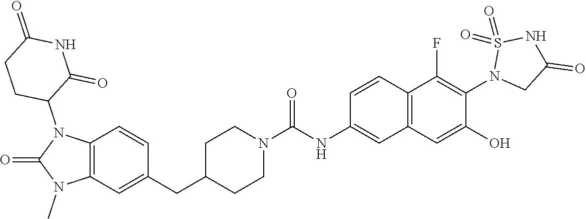 [SMILES: Cn1c(=O)n(C2CCC(=O)NC2=O)c2ccc(CC3CCN(C(=O)Nc4ccc5c(F)c(N6CC(=O)NS6(=O)=O)c(O)cc5c4)CC3)cc21] |
| 35 | 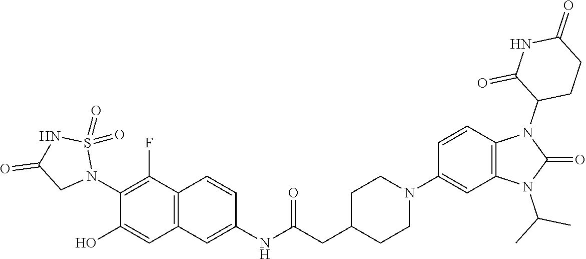 [SMILES: CC(C)n1c(=O)n(C2CCC(=O)NC2=O)c2ccc(N3CCC(CC(=O)Nc4ccc5c(F)c(N6CC(=O)NS6(=O)=O)c(O)cc5c4)CC3)cc21] |
| 36 | 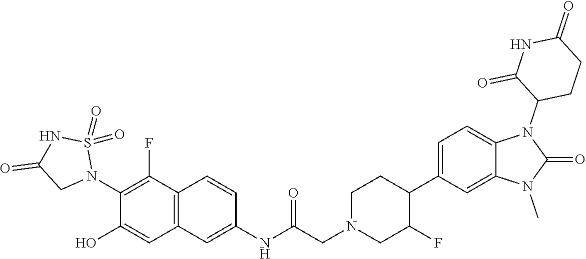 [SMILES: Cn1c(=O)n(C2CCC(=O)NC2=O)c2ccc(C3CCN(CC(=O)Nc4ccc5c(F)c(N6CC(=O)NS6(=O)=O)c(O)cc5c4)CC3F)cc21] |
| 37 | 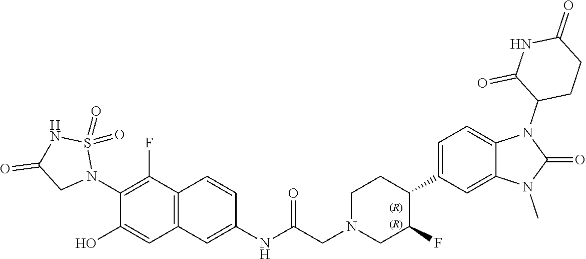 |
| 38 | 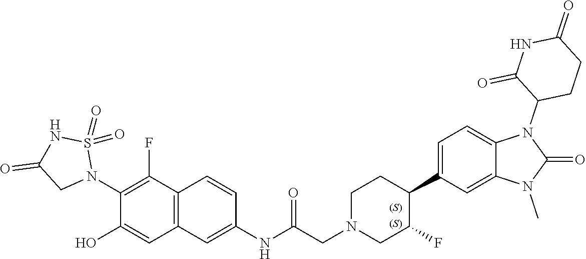 |
| 39 | 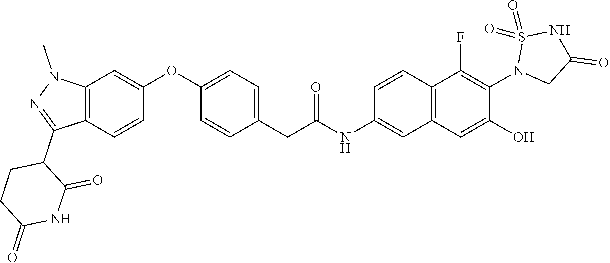 [SMILES: Cn1nc(C2CCC(=O)NC2=O)c2ccc(Oc3ccc(CC(=O)Nc4ccc5c(F)c(N6CC(=O)NS6(=O)=O)c(O)cc5c4)cc3)cc21] |
| 40 | 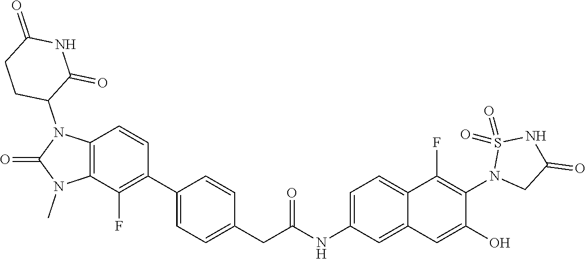 [SMILES: Cn1c(=O)n(C2CCC(=O)NC2=O)c2ccc(-c3ccc(CC(=O)Nc4ccc5c(F)c(N6CC(=O)NS6(=O)=O)c(O)cc5c4)cc3)c(F)c21] |
| 41 | 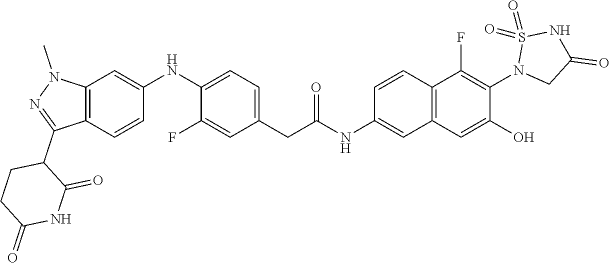 [SMILES: Cn1nc(C2CCC(=O)NC2=O)c2ccc(Nc3ccc(CC(=O)Nc4ccc5c(F)c(N6CC(=O)NS6(=O)=O)c(O)cc5c4)cc3F)cc21] |
| 42 | 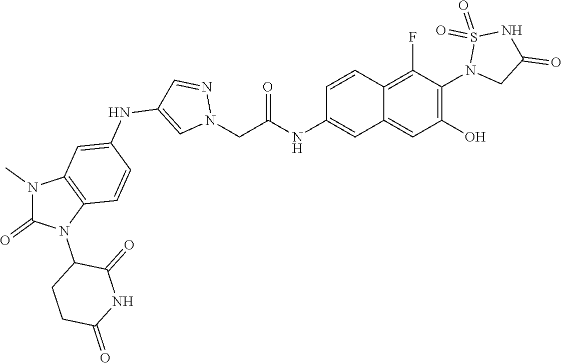 [SMILES: Cn1c(=O)n(C2CCC(=O)NC2=O)c2ccc(Nc3cnn(CC(=O)Nc4ccc5c(F)c(N6CC(=O)NS6(=O)=O)c(O)cc5c4)c3)cc21] |
| 43 | 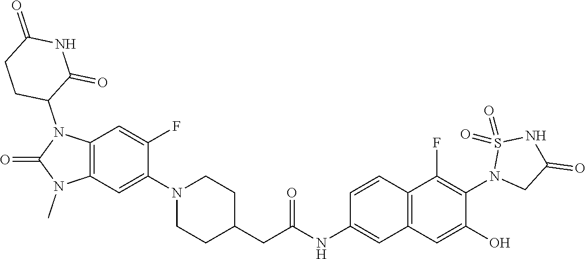 [SMILES: Cn1c(=O)n(C2CCC(=O)NC2=O)c2cc(F)c(N3CCC(CC(=O)Nc4ccc5c(F)c(N6CC(=O)NS6(=O)=O)c(O)cc5c4)CC3)cc21] |
| 44 | 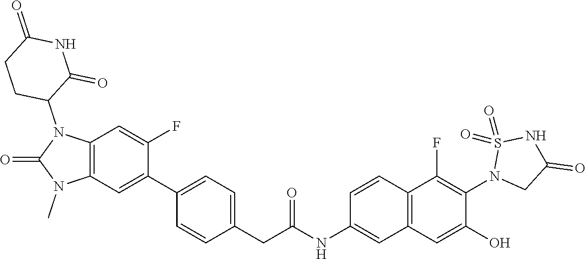 [SMILES: Cn1c(=O)n(C2CCC(=O)NC2=O)c2cc(F)c(-c3ccc(CC(=O)Nc4ccc5c(F)c(N6CC(=O)NS6(=O)=O)c(O)cc5c4)cc3)cc21] |
| 45 | 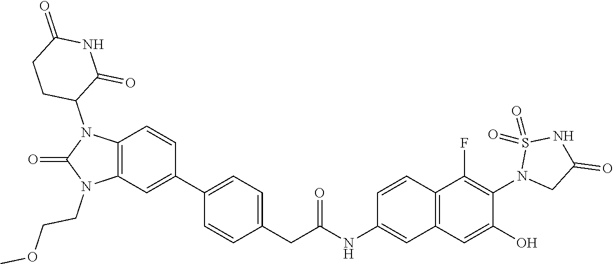 [SMILES: COCCn1c(=O)n(C2CCC(=O)NC2=O)c2ccc(-c3ccc(CC(=O)Nc4ccc5c(F)c(N6CC(=O)NS6(=O)=O)c(O)cc5c4)cc3)cc21] |
| 46 | 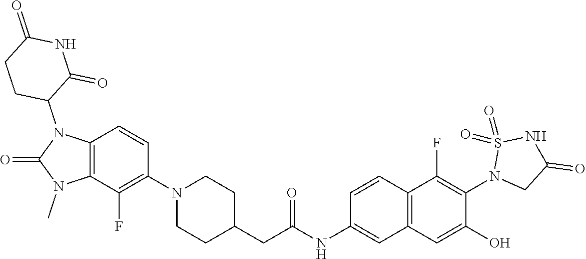 [SMILES: Cn1c(=O)n(C2CCC(=O)NC2=O)c2ccc(N3CCC(CC(=O)Nc4ccc5c(F)c(N6CC(=O)NS6(=O)=O)c(O)cc5c4)CC3)c(F)c21] |
| 47 | 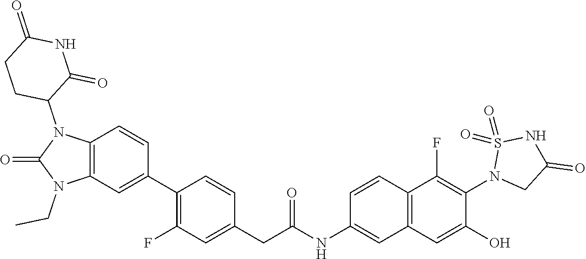 [SMILES: CCn1c(=O)n(C2CCC(=O)NC2=O)c2ccc(-c3ccc(CC(=O)Nc4ccc5c(F)c(N6CC(=O)NS6(=O)=O)c(O)cc5c4)cc3F)cc21] |
| 48 | 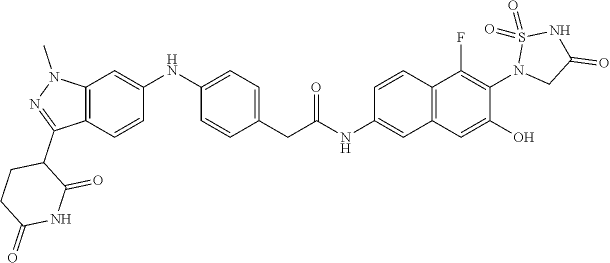 [SMILES: Cn1nc(C2CCC(=O)NC2=O)c2ccc(Nc3ccc(CC(=O)Nc4ccc5c(F)c(N6CC(=O)NS6(=O)=O)c(O)cc5c4)cc3)cc21] |
| 49 | 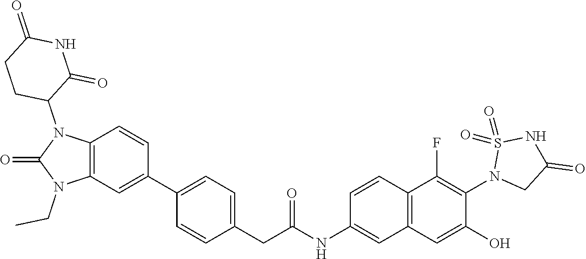 [SMILES: CCn1c(=O)n(C2CCC(=O)NC2=O)c2ccc(-c3ccc(CC(=O)Nc4ccc5c(F)c(N6CC(=O)NS6(=O)=O)c(O)cc5c4)cc3)cc21] |
| 50 | 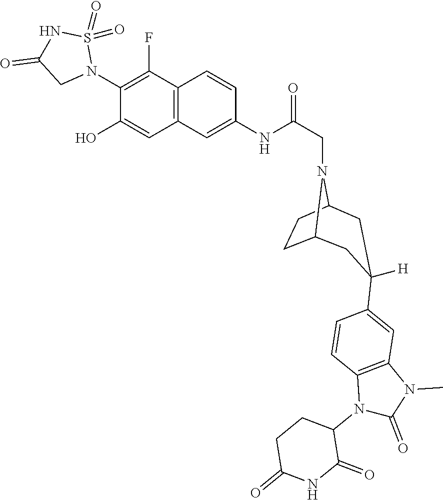 [SMILES: Cn1c(=O)n(C2CCC(=O)NC2=O)c2ccc(C3CC4CCC(C3)N4CC(=O)Nc3ccc4c(F)c(N5CC(=O)NS5(=O)=O)c(O)cc4c3)cc21] |
| 51 | 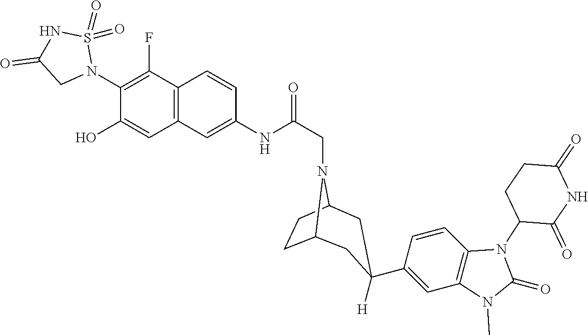 [SMILES: Cn1c(=O)n(C2CCC(=O)NC2=O)c2ccc(C3CC4CCC(C3)N4CC(=O)Nc3ccc4c(F)c(N5CC(=O)NS5(=O)=O)c(O)cc4c3)cc21] |
| 52 | 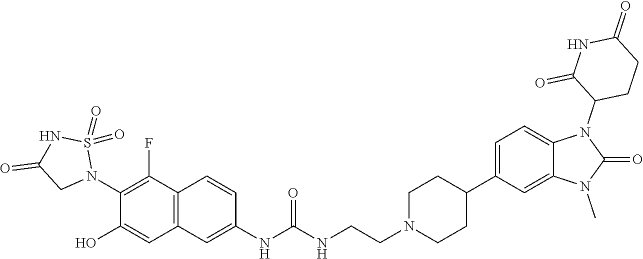 [SMILES: Cn1c(=O)n(C2CCC(=O)NC2=O)c2ccc(C3CCN(CCNC(=O)Nc4ccc5c(F)c(N6CC(=O)NS6(=O)=O)c(O)cc5c4)CC3)cc21] |
| 53 | 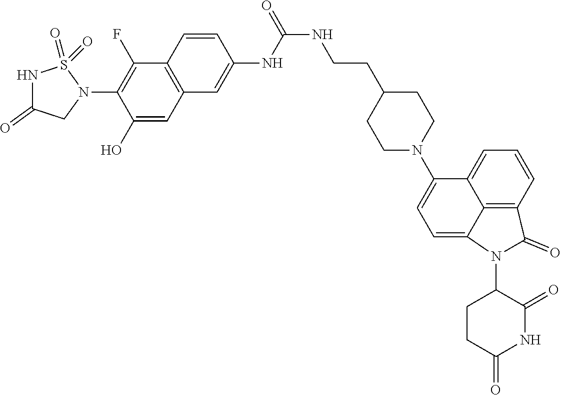 [SMILES: O=C1CCC(N2C(=O)c3cccc4c(N5CCC(CCNC(=O)Nc6ccc7c(F)c(N8CC(=O)NS8(=O)=O)c(O)cc7c6)CC5)ccc2c34)C(=O)N1] |
| 54 | 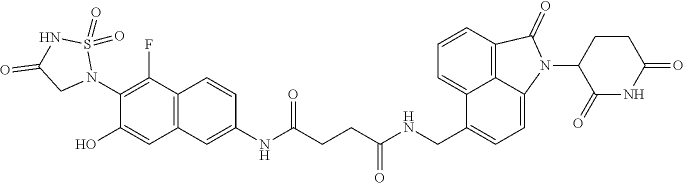 [SMILES: O=C(CCC(=O)Nc1ccc2c(F)c(N3CC(=O)NS3(=O)=O)c(O)cc2c1)NCc1ccc2c3c(cccc13)C(=O)N2C1CCC(=O)NC1=O] |
| 55 | 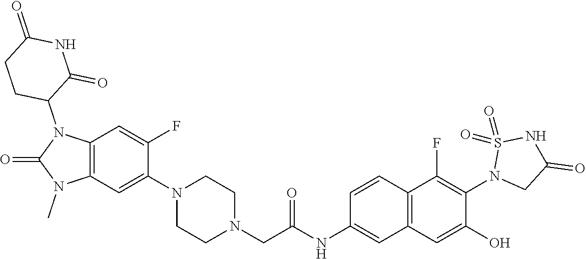 [SMILES: Cn1c(=O)n(C2CCC(=O)NC2=O)c2cc(F)c(N3CCN(CC(=O)Nc4ccc5c(F)c(N6CC(=O)NS6(=O)=O)c(O)cc5c4)CC3)cc21] |
| 56 | 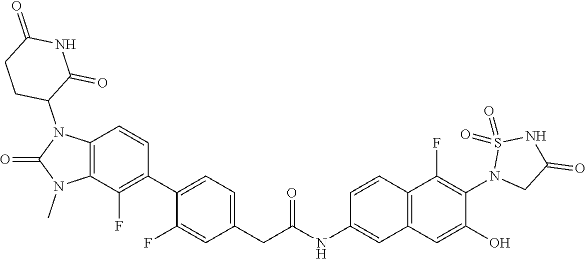 [SMILES: Cn1c(=O)n(C2CCC(=O)NC2=O)c2ccc(-c3ccc(CC(=O)Nc4ccc5c(F)c(N6CC(=O)NS6(=O)=O)c(O)cc5c4)cc3F)c(F)c21] |
| 57 | 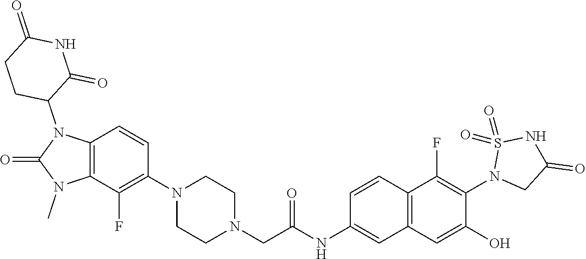 [SMILES: Cn1c(=O)n(C2CCC(=O)NC2=O)c2ccc(N3CCN(CC(=O)Nc4ccc5c(F)c(N6CC(=O)NS6(=O)=O)c(O)cc5c4)CC3)c(F)c21] |
| 58 | 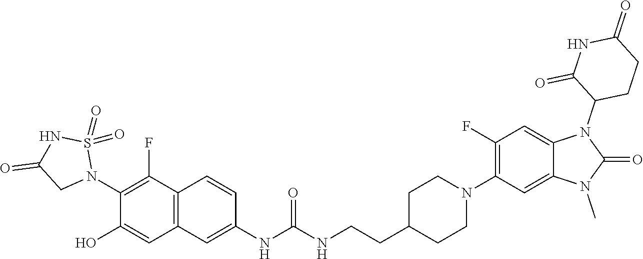 [SMILES: Cn1c(=O)n(C2CCC(=O)NC2=O)c2cc(F)c(N3CCC(CCNC(=O)Nc4ccc5c(F)c(N6CC(=O)NS6(=O)=O)c(O)cc5c4)CC3)cc21] |
| 59 | 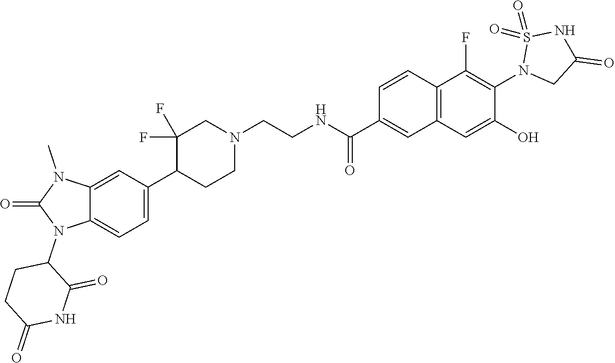 [SMILES: Cn1c(=O)n(C2CCC(=O)NC2=O)c2ccc(C3CCN(CCNC(=O)c4ccc5c(F)c(N6CC(=O)NS6(=O)=O)c(O)cc5c4)CC3(F)F)cc21] |
| 60 | 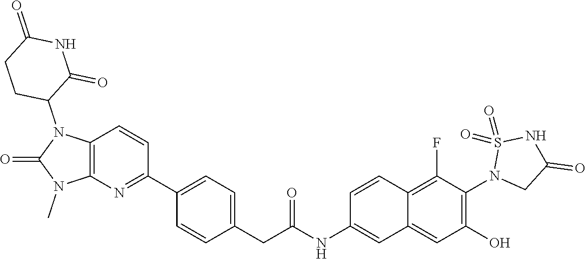 [SMILES: Cn1c(=O)n(C2CCC(=O)NC2=O)c2ccc(-c3ccc(CC(=O)Nc4ccc5c(F)c(N6CC(=O)NS6(=O)=O)c(O)cc5c4)cc3)nc21] |
| 61 | 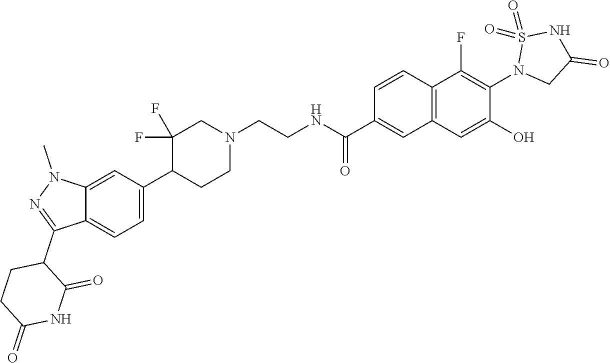 [SMILES: Cn1nc(C2CCC(=O)NC2=O)c2ccc(C3CCN(CCNC(=O)c4ccc5c(F)c(N6CC(=O)NS6(=O)=O)c(O)cc5c4)CC3(F)F)cc21] |
| 62 | 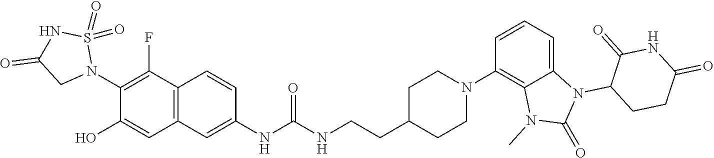 [SMILES: Cn1c(=O)n(C2CCC(=O)NC2=O)c2cccc(N3CCC(CCNC(=O)Nc4ccc5c(F)c(N6CC(=O)NS6(=O)=O)c(O)cc5c4)CC3)c21] |
| 63 | 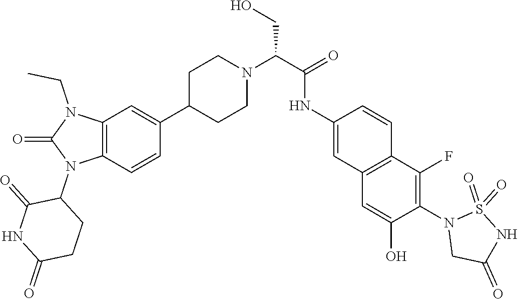 [SMILES: CCn1c(=O)n(C2CCC(=O)NC2=O)c2ccc(C3CCN([C@H](CO)C(=O)Nc4ccc5c(F)c(N6CC(=O)NS6(=O)=O)c(O)cc5c4)CC3)cc21] |
| 64 | 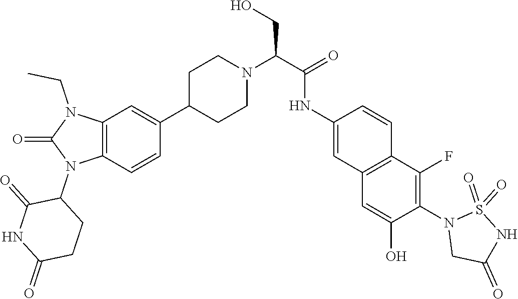 [SMILES: CCn1c(=O)n(C2CCC(=O)NC2=O)c2ccc(C3CCN([C@@H](CO)C(=O)Nc4ccc5c(F)c(N6CC(=O)NS6(=O)=O)c(O)cc5c4)CC3)cc21] |
| 65 | 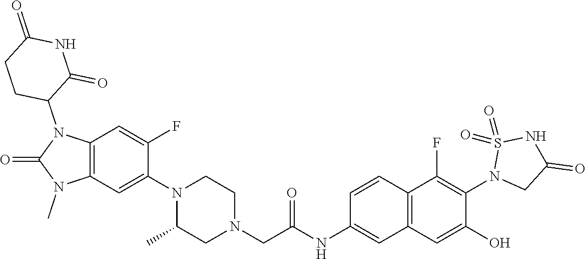 [SMILES: C[C@H]1CN(CC(=O)Nc2ccc3c(F)c(N4CC(=O)NS4(=O)=O)c(O)cc3c2)CCN1c1cc2c(cc1F)n(C1CCC(=O)NC1=O)c(=O)n2C] |
| 66 | 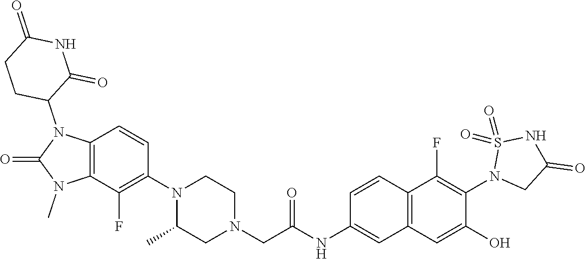 [SMILES: C[C@H]1CN(CC(=O)Nc2ccc3c(F)c(N4CC(=O)NS4(=O)=O)c(O)cc3c2)CCN1c1ccc2c(c1F)n(C)c(=O)n2C1CCC(=O)NC1=O] |
| 67 | 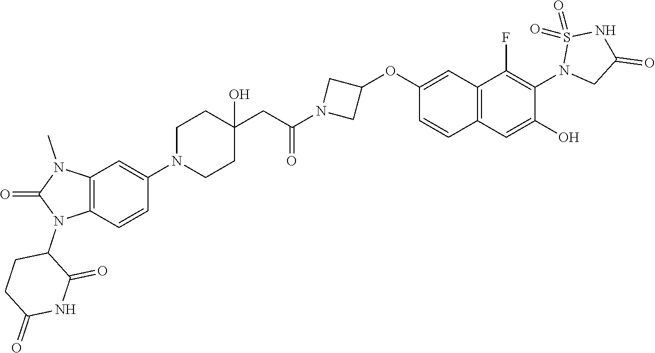 [SMILES: Cn1c(=O)n(C2CCC(=O)NC2=O)c2ccc(N3CCC(O)(CC(=O)N4CC(Oc5ccc6cc(O)c(N7CC(=O)NS7(=O)=O)c(F)c6c5)C4)CC3)cc21] |
| 68 | 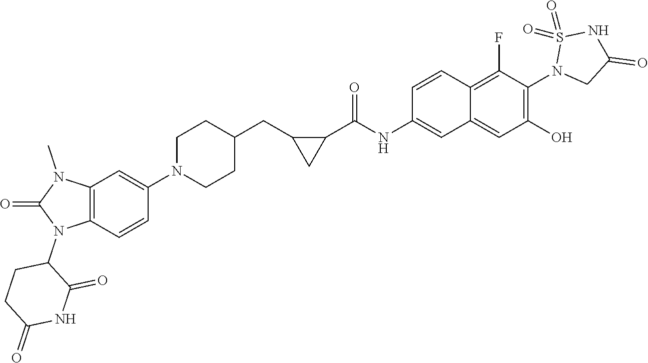 [SMILES: Cn1c(=O)n(C2CCC(=O)NC2=O)c2ccc(N3CCC(CC4CC4C(=O)Nc4ccc5c(F)c(N6CC(=O)NS6(=O)=O)c(O)cc5c4)CC3)cc21] |
| 69 | 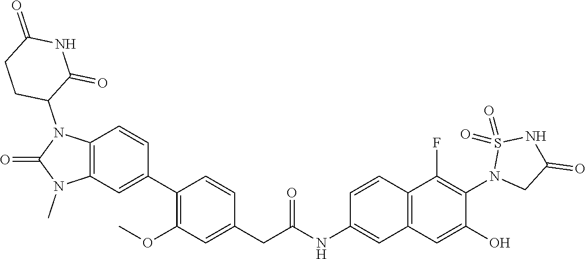 [SMILES: COc1cc(CC(=O)Nc2ccc3c(F)c(N4CC(=O)NS4(=O)=O)c(O)cc3c2)ccc1-c1ccc2c(c1)n(C)c(=O)n2C1CCC(=O)NC1=O] |
| 70 | 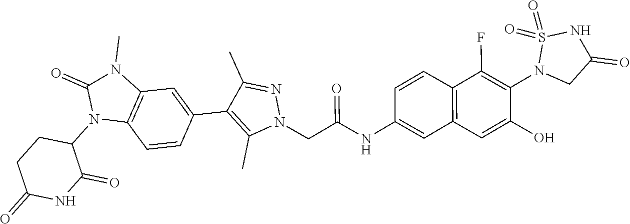 [SMILES: Cc1nn(CC(=O)Nc2ccc3c(F)c(N4CC(=O)NS4(=O)=O)c(O)cc3c2)c(C)c1-c1ccc2c(c1)n(C)c(=O)n2C1CCC(=O)NC1=O] |
| 71 | 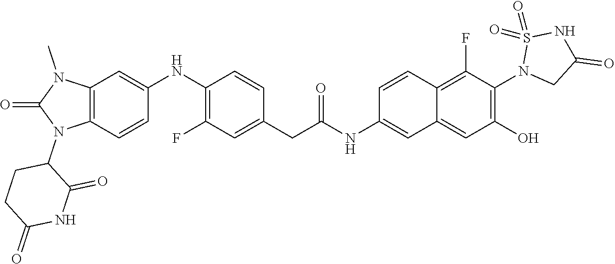 [SMILES: Cn1c(=O)n(C2CCC(=O)NC2=O)c2ccc(Nc3ccc(CC(=O)Nc4ccc5c(F)c(N6CC(=O)NS6(=O)=O)c(O)cc5c4)cc3F)cc21] |
| 72 | 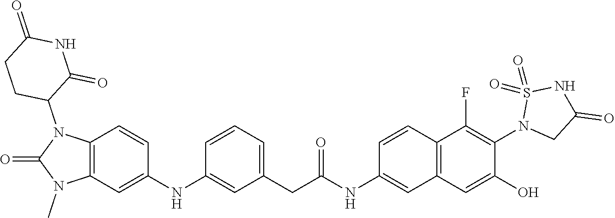 [SMILES: Cn1c(=O)n(C2CCC(=O)NC2=O)c2ccc(Nc3cccc(CC(=O)Nc4ccc5c(F)c(N6CC(=O)NS6(=O)=O)c(O)cc5c4)c3)cc21] |
| 73 | 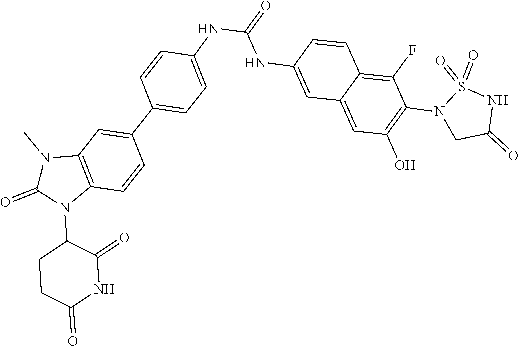 [SMILES: Cn1c(=O)n(C2CCC(=O)NC2=O)c2ccc(-c3ccc(NC(=O)Nc4ccc5c(F)c(N6CC(=O)NS6(=O)=O)c(O)cc5c4)cc3)cc21] |
| 74 | 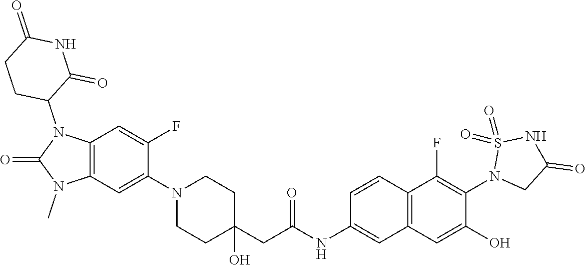 [SMILES: Cn1c(=O)n(C2CCC(=O)NC2=O)c2cc(F)c(N3CCC(O)(CC(=O)Nc4ccc5c(F)c(N6CC(=O)NS6(=O)=O)c(O)cc5c4)CC3)cc21] |
| 75 | 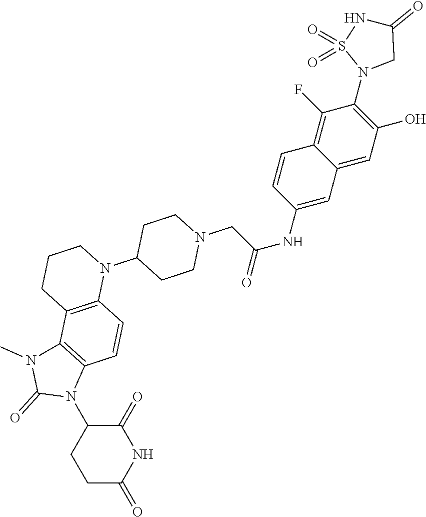 [SMILES: Cn1c(=O)n(C2CCC(=O)NC2=O)c2ccc3c(c21)CCCN3C1CCN(CC(=O)Nc2ccc3c(F)c(N4CC(=O)NS4(=O)=O)c(O)cc3c2)CC1] |
| 76 | 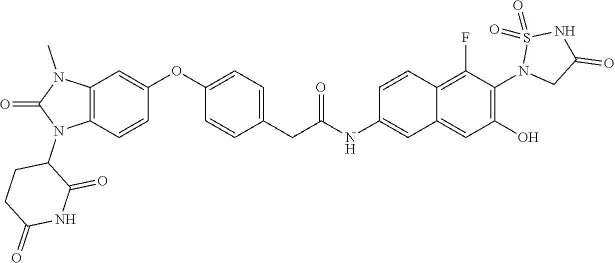 [SMILES: Cn1c(=O)n(C2CCC(=O)NC2=O)c2ccc(Oc3ccc(CC(=O)Nc4ccc5c(F)c(N6CC(=O)NS6(=O)=O)c(O)cc5c4)cc3)cc21] |
| 77 | 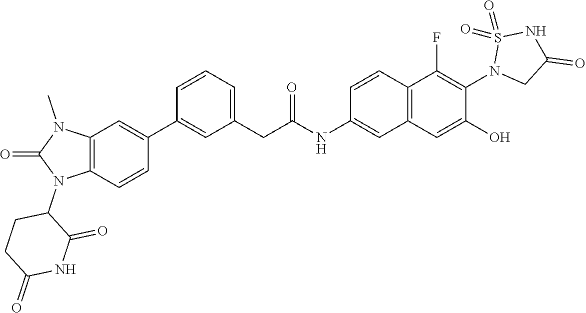 [SMILES: Cn1c(=O)n(C2CCC(=O)NC2=O)c2ccc(-c3cccc(CC(=O)Nc4ccc5c(F)c(N6CC(=O)NS6(=O)=O)c(O)cc5c4)c3)cc21] |
| 78 | 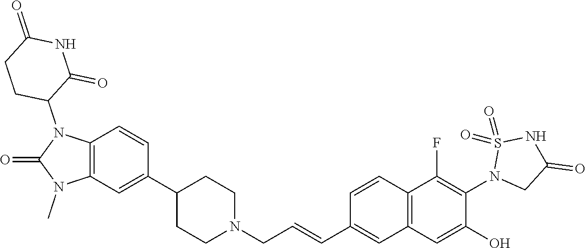 [SMILES: Cn1c(=O)n(C2CCC(=O)NC2=O)c2ccc(C3CCN(C/C=C/c4ccc5c(F)c(N6CC(=O)NS6(=O)=O)c(O)cc5c4)CC3)cc21] |
| 79 | 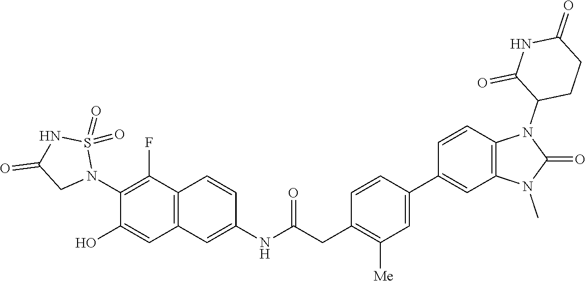 [SMILES: Cc1cc(-c2ccc3c(c2)n(C)c(=O)n3C2CCC(=O)NC2=O)ccc1CC(=O)Nc1ccc2c(F)c(N3CC(=O)NS3(=O)=O)c(O)cc2c1] |
| 80 | 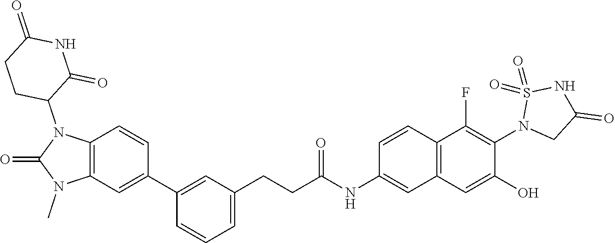 [SMILES: Cn1c(=O)n(C2CCC(=O)NC2=O)c2ccc(-c3cccc(CCC(=O)Nc4ccc5c(F)c(N6CC(=O)NS6(=O)=O)c(O)cc5c4)c3)cc21] |
| 81 | 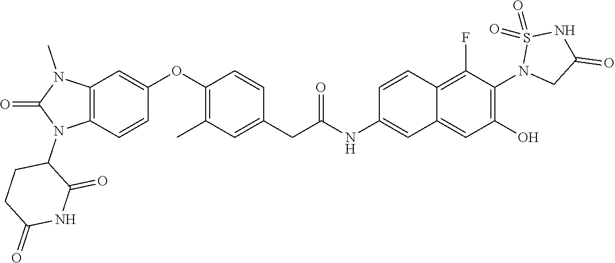 [SMILES: Cc1cc(CC(=O)Nc2ccc3c(F)c(N4CC(=O)NS4(=O)=O)c(O)cc3c2)ccc1Oc1ccc2c(c1)n(C)c(=O)n2C1CCC(=O)NC1=O] |
| 82 | 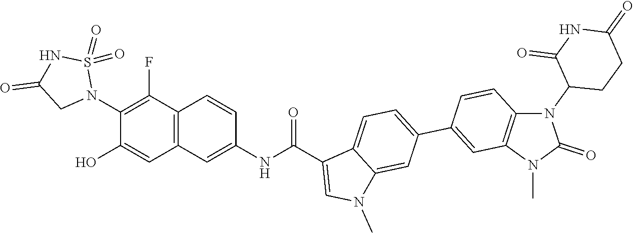 [SMILES: Cn1cc(C(=O)Nc2ccc3c(F)c(N4CC(=O)NS4(=O)=O)c(O)cc3c2)c2ccc(-c3ccc4c(c3)n(C)c(=O)n4C3CCC(=O)NC3=O)cc21] |
| 83 | 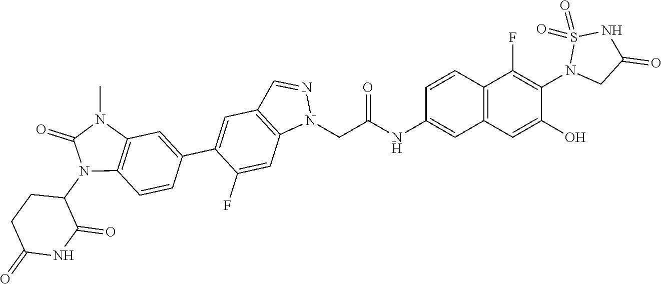 [SMILES: Cn1c(=O)n(C2CCC(=O)NC2=O)c2ccc(-c3cc4cnn(CC(=O)Nc5ccc6c(F)c(N7CC(=O)NS7(=O)=O)c(O)cc6c5)c4cc3F)cc21] |
| 84 | 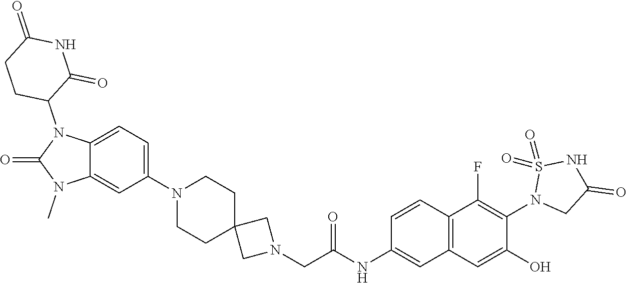 [SMILES: Cn1c(=O)n(C2CCC(=O)NC2=O)c2ccc(N3CCC4(CC3)CN(CC(=O)Nc3ccc5c(F)c(N6CC(=O)NS6(=O)=O)c(O)cc5c3)C4)cc21] |
| 85 | 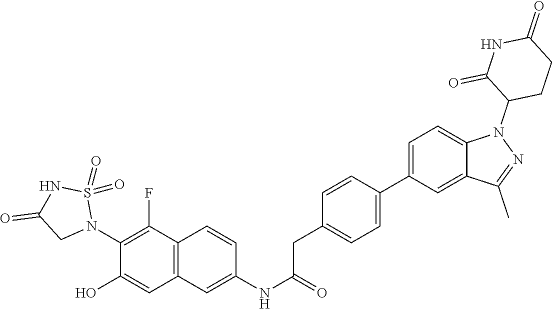 [SMILES: Cc1nn(C2CCC(=O)NC2=O)c2ccc(-c3ccc(CC(=O)Nc4ccc5c(F)c(N6CC(=O)NS6(=O)=O)c(O)cc5c4)cc3)cc12] |
| 86 | 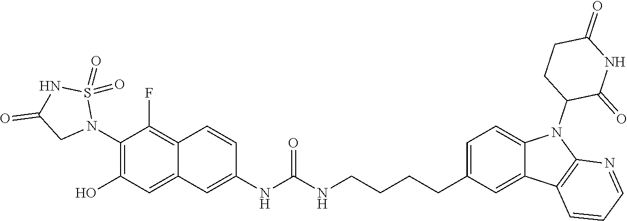 [SMILES: O=C1CCC(n2c3ccc(CCCCNC(=O)Nc4ccc5c(F)c(N6CC(=O)NS6(=O)=O)c(O)cc5c4)cc3c3cccnc32)C(=O)N1] |
| 87 | 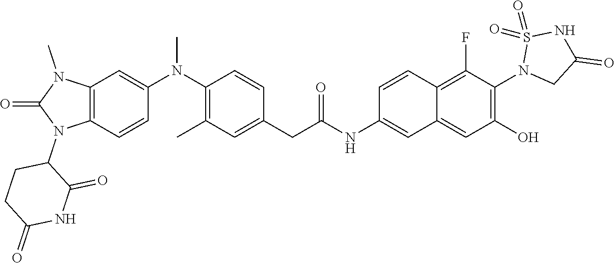 [SMILES: Cc1cc(CC(=O)Nc2ccc3c(F)c(N4CC(=O)NS4(=O)=O)c(O)cc3c2)ccc1N(C)c1ccc2c(c1)n(C)c(=O)n2C1CCC(=O)NC1=O] |
| 88 | 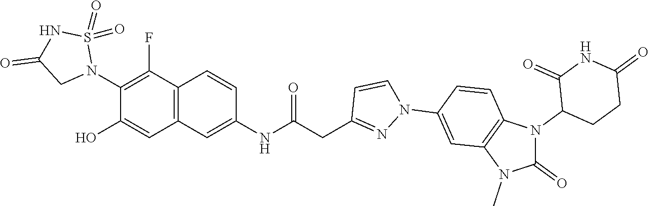 [SMILES: Cn1c(=O)n(C2CCC(=O)NC2=O)c2ccc(-n3ccc(CC(=O)Nc4ccc5c(F)c(N6CC(=O)NS6(=O)=O)c(O)cc5c4)n3)cc21] |
| 89 | 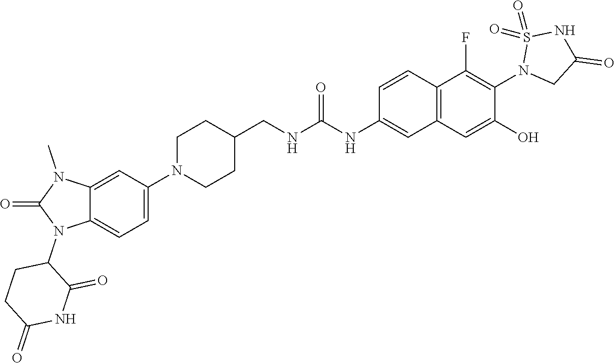 [SMILES: Cn1c(=O)n(C2CCC(=O)NC2=O)c2ccc(N3CCC(CNC(=O)Nc4ccc5c(F)c(N6CC(=O)NS6(=O)=O)c(O)cc5c4)CC3)cc21] |
| 90 | 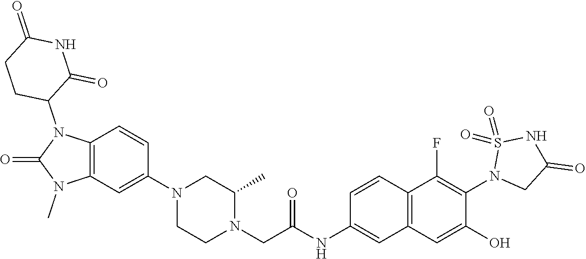 [SMILES: C[C@H]1CN(c2ccc3c(c2)n(C)c(=O)n3C2CCC(=O)NC2=O)CCN1CC(=O)Nc1ccc2c(F)c(N3CC(=O)NS3(=O)=O)c(O)cc2c1] |
| 91 | 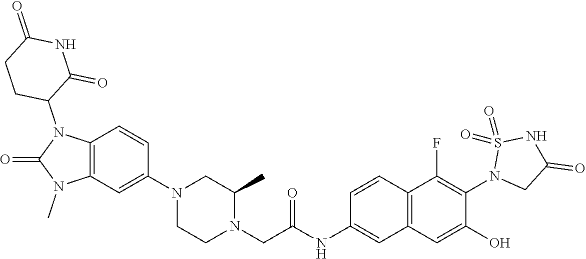 [SMILES: C[C@@H]1CN(c2ccc3c(c2)n(C)c(=O)n3C2CCC(=O)NC2=O)CCN1CC(=O)Nc1ccc2c(F)c(N3CC(=O)NS3(=O)=O)c(O)cc2c1] |
| 92 | 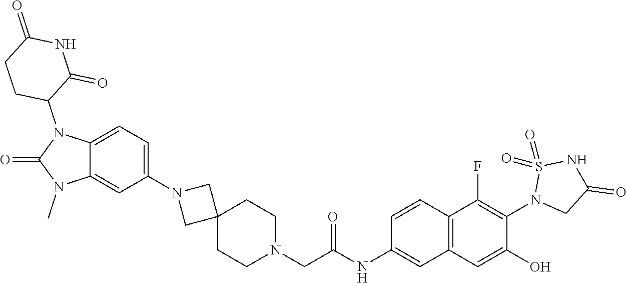 [SMILES: Cn1c(=O)n(C2CCC(=O)NC2=O)c2ccc(N3CC4(CCN(CC(=O)Nc5ccc6c(F)c(N7CC(=O)NS7(=O)=O)c(O)cc6c5)CC4)C3)cc21] |
| 93 | 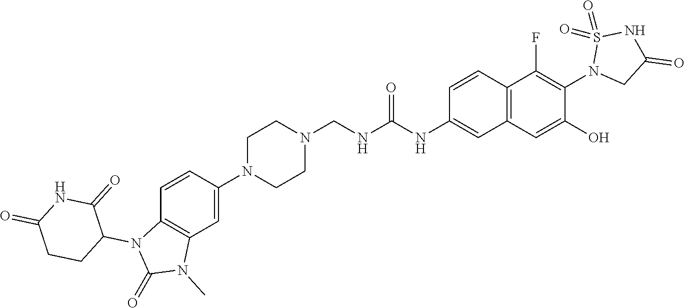 [SMILES: Cn1c(=O)n(C2CCC(=O)NC2=O)c2ccc(N3CCN(CNC(=O)Nc4ccc5c(F)c(N6CC(=O)NS6(=O)=O)c(O)cc5c4)CC3)cc21] |
| 94 | 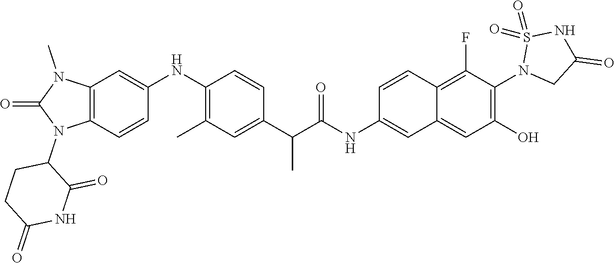 [SMILES: Cc1cc(C(C)C(=O)Nc2ccc3c(F)c(N4CC(=O)NS4(=O)=O)c(O)cc3c2)ccc1Nc1ccc2c(c1)n(C)c(=O)n2C1CCC(=O)NC1=O] |
| 95 | 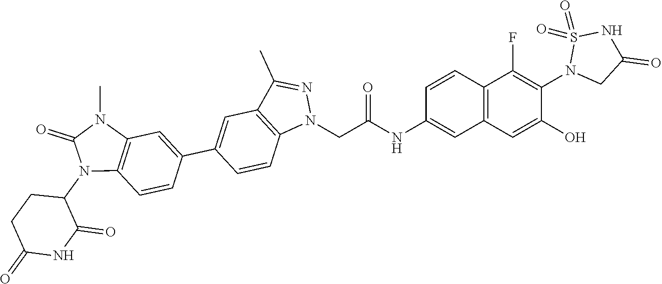 [SMILES: Cc1nn(CC(=O)Nc2ccc3c(F)c(N4CC(=O)NS4(=O)=O)c(O)cc3c2)c2ccc(-c3ccc4c(c3)n(C)c(=O)n4C3CCC(=O)NC3=O)cc12] |
| 96 | 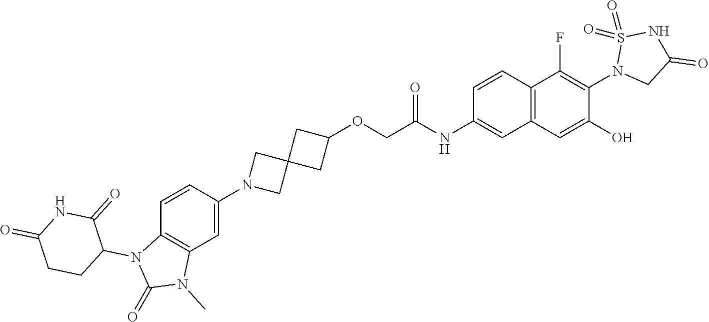 [SMILES: Cn1c(=O)n(C2CCC(=O)NC2=O)c2ccc(N3CC4(CC(OCC(=O)Nc5ccc6c(F)c(N7CC(=O)NS7(=O)=O)c(O)cc6c5)C4)C3)cc21] |
| 97 | 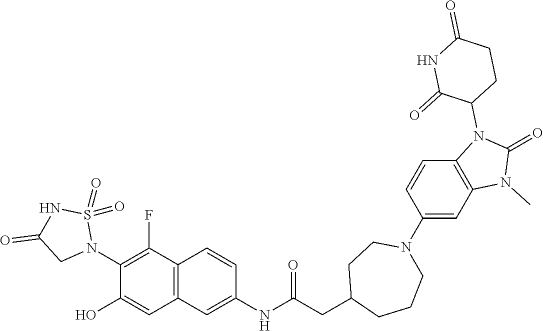 [SMILES: Cn1c(=O)n(C2CCC(=O)NC2=O)c2ccc(N3CCCC(CC(=O)Nc4ccc5c(F)c(N6CC(=O)NS6(=O)=O)c(O)cc5c4)CC3)cc21] |
| 98 | 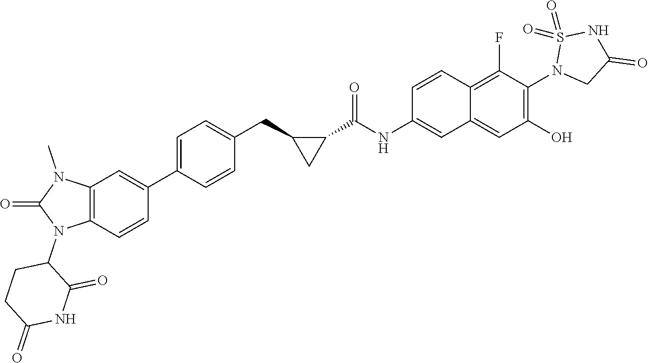 [SMILES: Cn1c(=O)n(C2CCC(=O)NC2=O)c2ccc(-c3ccc(C[C@@H]4C[C@H]4C(=O)Nc4ccc5c(F)c(N6CC(=O)NS6(=O)=O)c(O)cc5c4)cc3)cc21] |
| 99 | 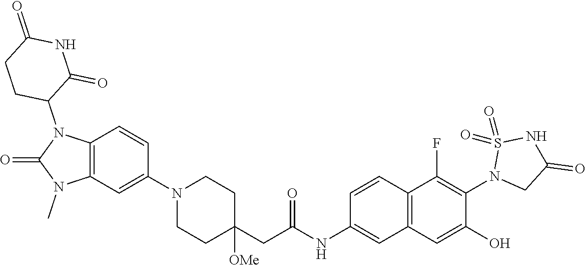 [SMILES: COC1(CC(=O)Nc2ccc3c(F)c(N4CC(=O)NS4(=O)=O)c(O)cc3c2)CCN(c2ccc3c(c2)n(C)c(=O)n3C2CCC(=O)NC2=O)CC1] |
| 100 | 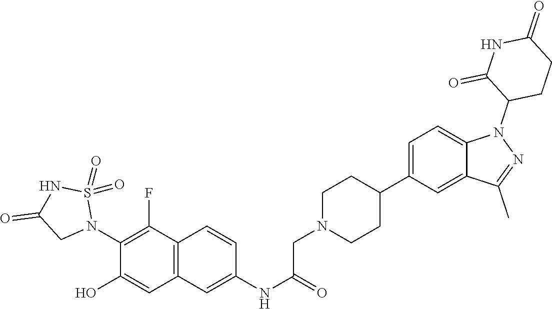 [SMILES: Cc1nn(C2CCC(=O)NC2=O)c2ccc(C3CCN(CC(=O)Nc4ccc5c(F)c(N6CC(=O)NS6(=O)=O)c(O)cc5c4)CC3)cc12] |
| 101 | 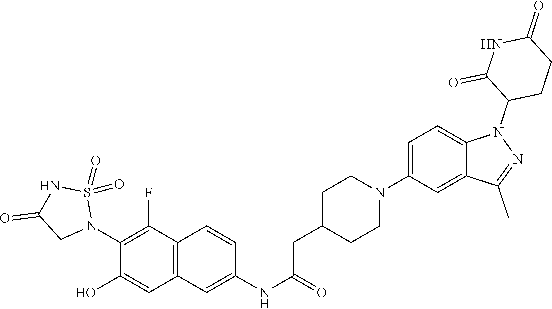 [SMILES: Cc1nn(C2CCC(=O)NC2=O)c2ccc(N3CCC(CC(=O)Nc4ccc5c(F)c(N6CC(=O)NS6(=O)=O)c(O)cc5c4)CC3)cc12] |
| 102 | 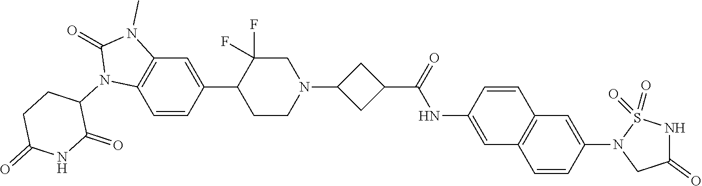 [SMILES: Cn1c(=O)n(C2CCC(=O)NC2=O)c2ccc(C3CCN(C4CC(C(=O)Nc5ccc6cc(N7CC(=O)NS7(=O)=O)ccc6c5)C4)CC3(F)F)cc21] |
| 103 | 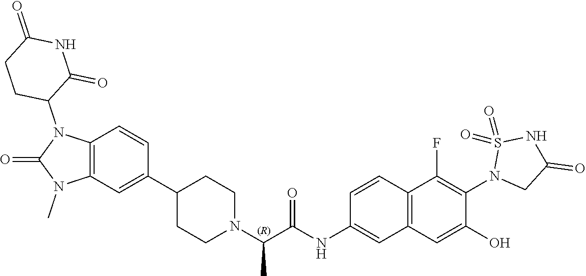 |
| 104 | 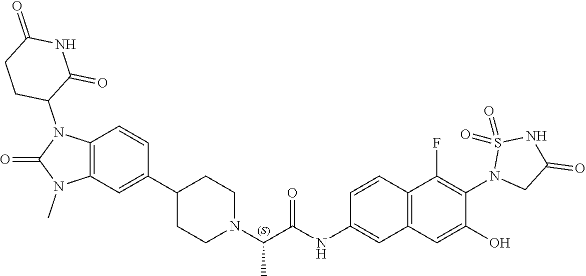 |
| 105 | 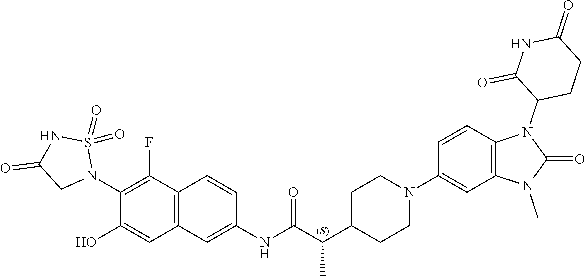 |
| 106 | 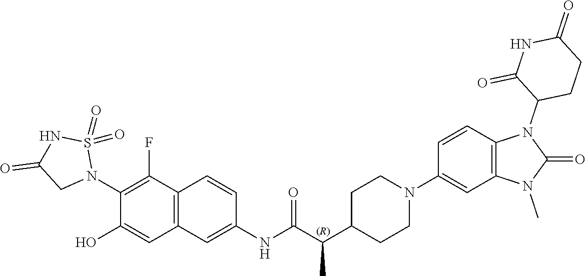 |
| 107 | 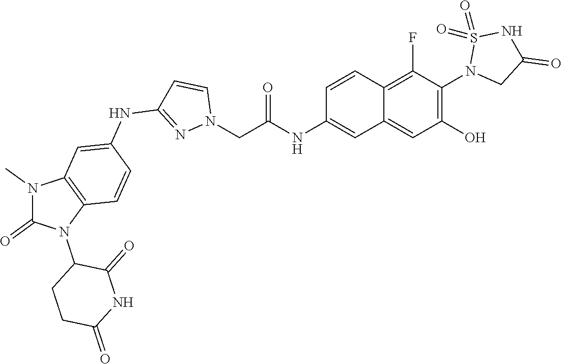 [SMILES: Cn1c(=O)n(C2CCC(=O)NC2=O)c2ccc(Nc3ccn(CC(=O)Nc4ccc5c(F)c(N6CC(=O)NS6(=O)=O)c(O)cc5c4)n3)cc21] |
| 108 | 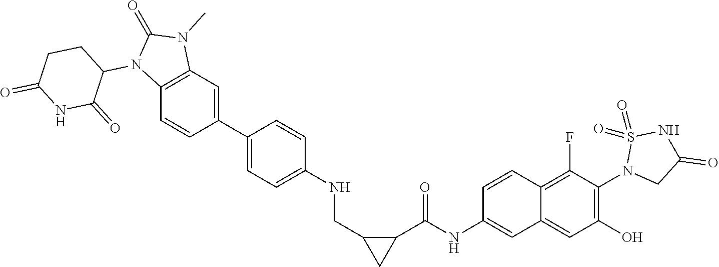 [SMILES: Cn1c(=O)n(C2CCC(=O)NC2=O)c2ccc(-c3ccc(NCC4CC4C(=O)Nc4ccc5c(F)c(N6CC(=O)NS6(=O)=O)c(O)cc5c4)cc3)cc21] |
| 109 | 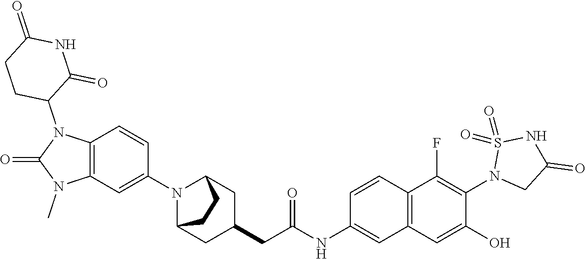 |
| 110 | 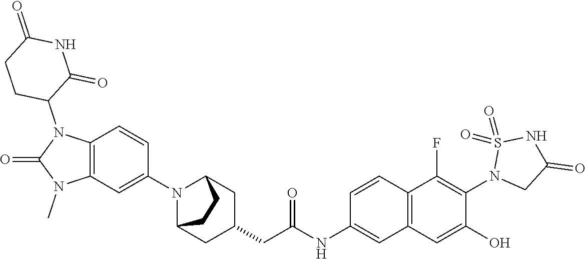 |
| 111 | 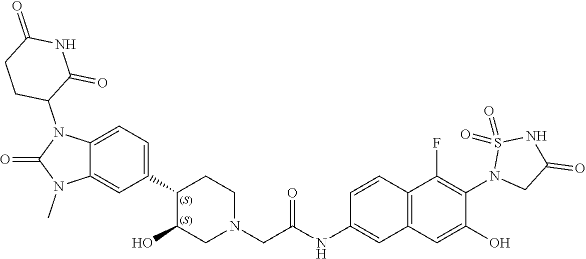 |
| 112 | 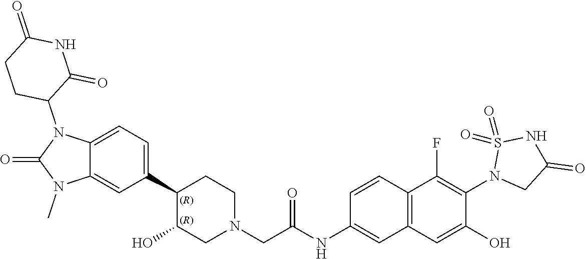 |
| 113 | 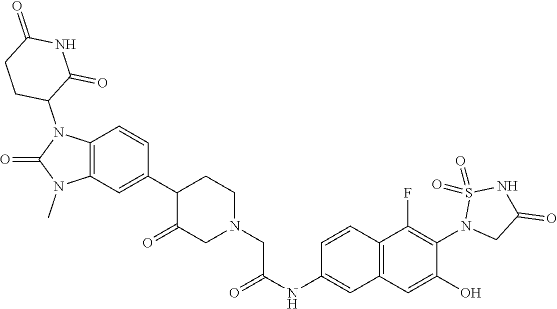 [SMILES: Cn1c(=O)n(C2CCC(=O)NC2=O)c2ccc(C3CCN(CC(=O)Nc4ccc5c(F)c(N6CC(=O)NS6(=O)=O)c(O)cc5c4)CC3=O)cc21] |
| 114 | 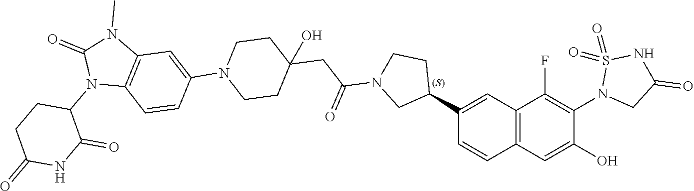 |
| 115 | 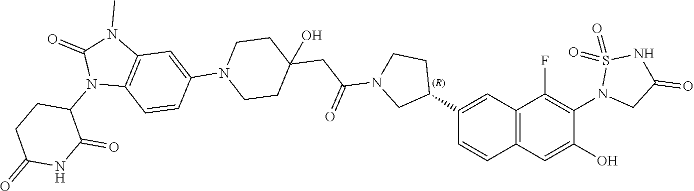 |
| 116 | 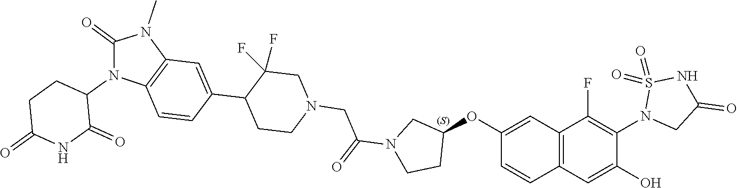 |
| 117 | 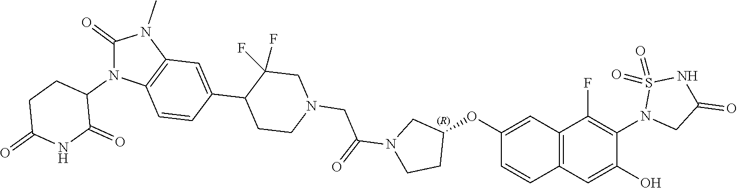 |
| 118 | 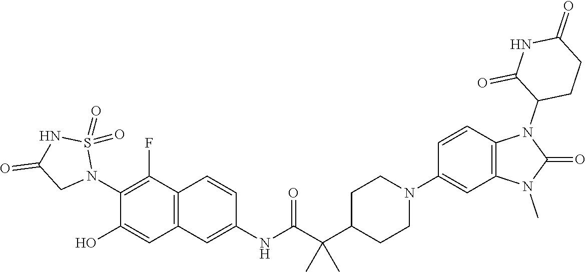 [SMILES: Cn1c(=O)n(C2CCC(=O)NC2=O)c2ccc(N3CCC(C(C)(C)C(=O)Nc4ccc5c(F)c(N6CC(=O)NS6(=O)=O)c(O)cc5c4)CC3)cc21] |
| 119 | 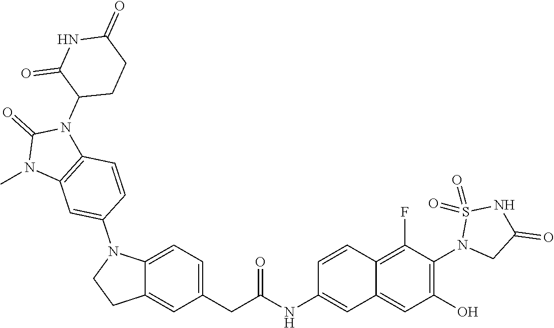 [SMILES: Cn1c(=O)n(C2CCC(=O)NC2=O)c2ccc(N3CCc4cc(CC(=O)Nc5ccc6c(F)c(N7CC(=O)NS7(=O)=O)c(O)cc6c5)ccc43)cc21] |
| 120 | 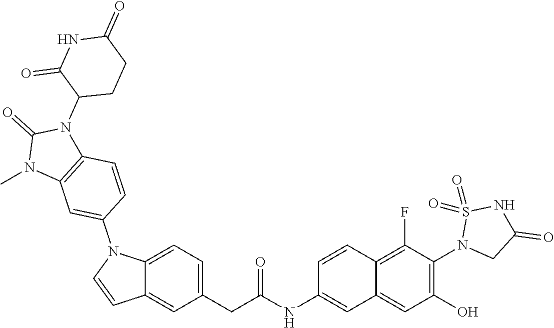 [SMILES: Cn1c(=O)n(C2CCC(=O)NC2=O)c2ccc(-n3ccc4cc(CC(=O)Nc5ccc6c(F)c(N7CC(=O)NS7(=O)=O)c(O)cc6c5)ccc43)cc21] |
| 121 | 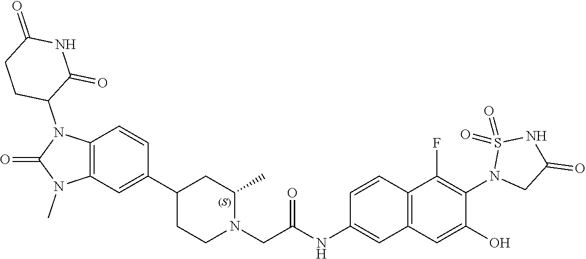 |
| 122 | 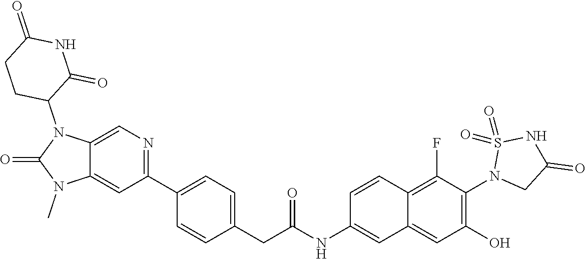 [SMILES: Cn1c(=O)n(C2CCC(=O)NC2=O)c2cnc(-c3ccc(CC(=O)Nc4ccc5c(F)c(N6CC(=O)NS6(=O)=O)c(O)cc5c4)cc3)cc21] |
| 123 | 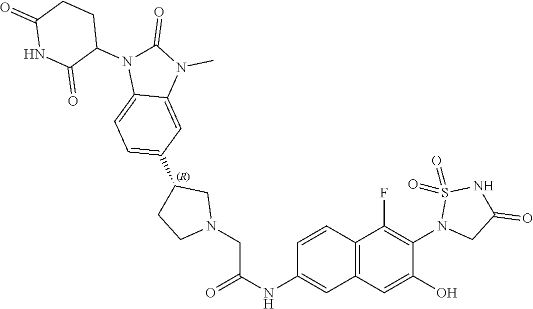 |
| 124 | 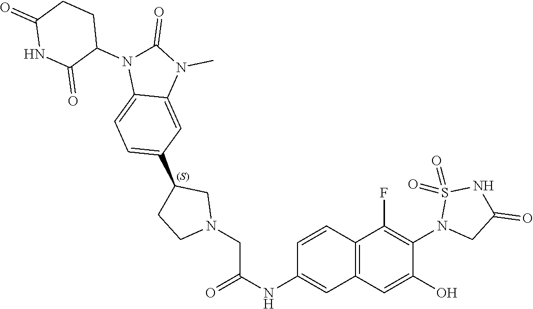 |
| 125 | 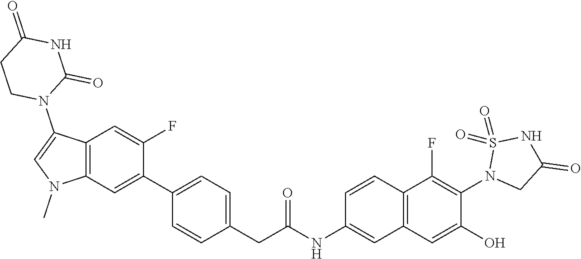 [SMILES: Cn1cc(N2CCC(=O)NC2=O)c2cc(F)c(-c3ccc(CC(=O)Nc4ccc5c(F)c(N6CC(=O)NS6(=O)=O)c(O)cc5c4)cc3)cc21] |
| 126 | 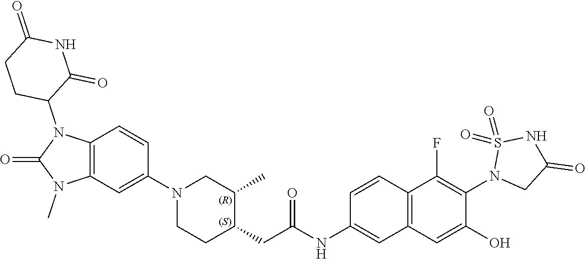 |
| 127 | 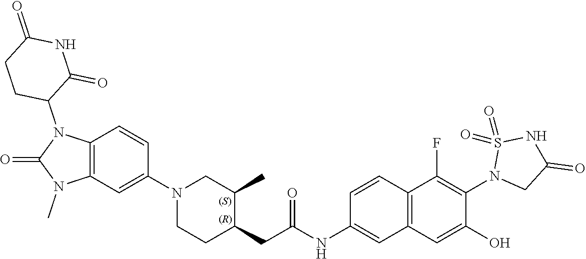 |
| 128 | 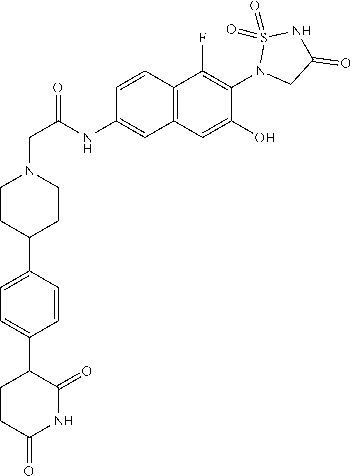 [SMILES: O=C1CCC(c2ccc(C3CCN(CC(=O)Nc4ccc5c(F)c(N6CC(=O)NS6(=O)=O)c(O)cc5c4)CC3)cc2)C(=O)N1] |
| 129 | 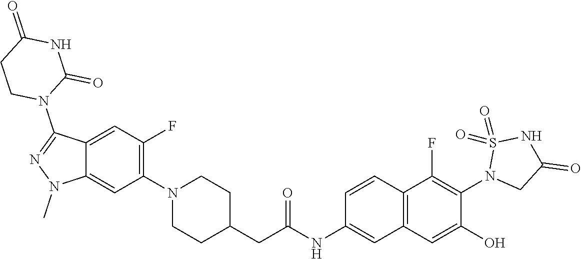 [SMILES: Cn1nc(N2CCC(=O)NC2=O)c2cc(F)c(N3CCC(CC(=O)Nc4ccc5c(F)c(N6CC(=O)NS6(=O)=O)c(O)cc5c4)CC3)cc21] |
| 130 | 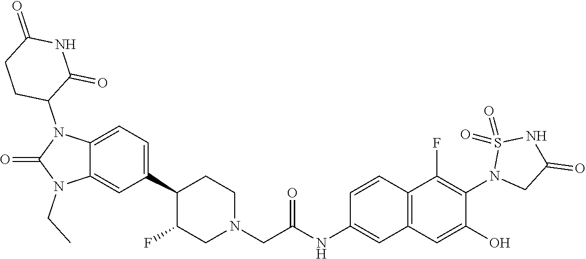 [SMILES: CCn1c(=O)n(C2CCC(=O)NC2=O)c2ccc([C@H]3CCN(CC(=O)Nc4ccc5c(F)c(N6CC(=O)NS6(=O)=O)c(O)cc5c4)C[C@@H]3F)cc21] |
| 131 | 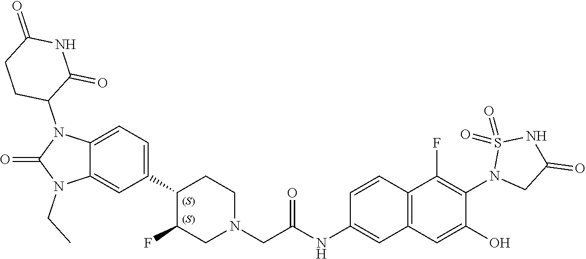 |
| 132 | 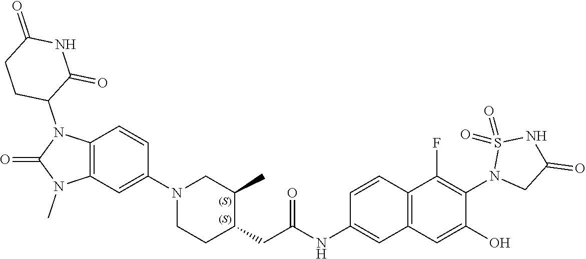 |
| 133 | 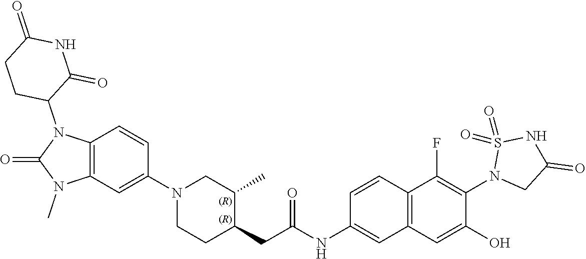 |
| 134 | 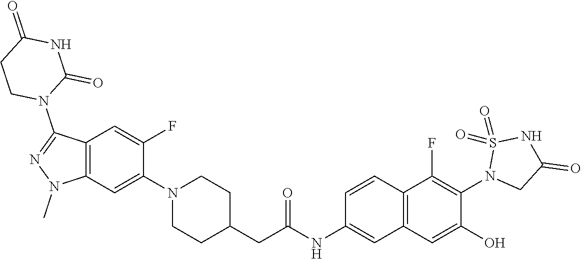 [SMILES: Cn1nc(N2CCC(=O)NC2=O)c2cc(F)c(N3CCC(CC(=O)Nc4ccc5c(F)c(N6CC(=O)NS6(=O)=O)c(O)cc5c4)CC3)cc21] |
| 135 | 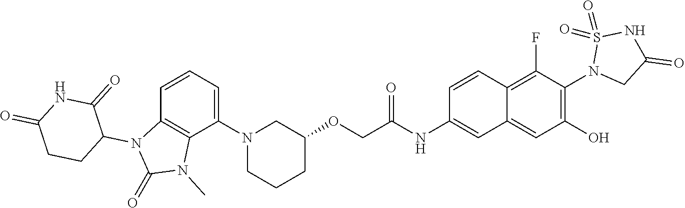 [SMILES: Cn1c(=O)n(C2CCC(=O)NC2=O)c2cccc(N3CCC[C@@H](OCC(=O)Nc4ccc5c(F)c(N6CC(=O)NS6(=O)=O)c(O)cc5c4)C3)c21] |
| 136 | 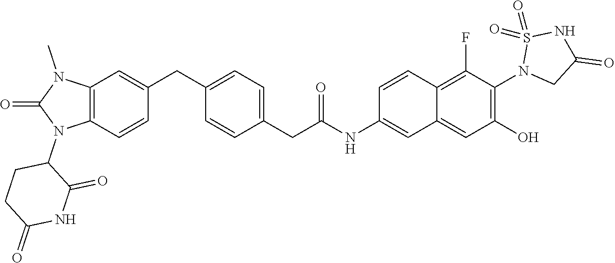 [SMILES: Cn1c(=O)n(C2CCC(=O)NC2=O)c2ccc(Cc3ccc(CC(=O)Nc4ccc5c(F)c(N6CC(=O)NS6(=O)=O)c(O)cc5c4)cc3)cc21] |
| 137 | 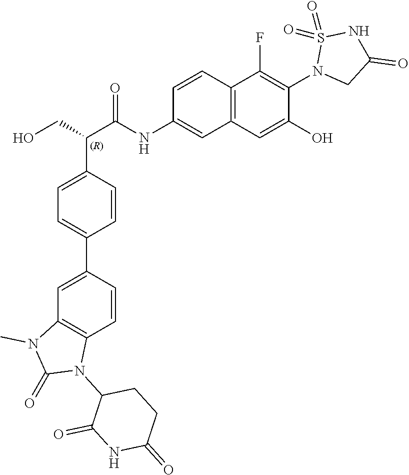 |
| 138 | 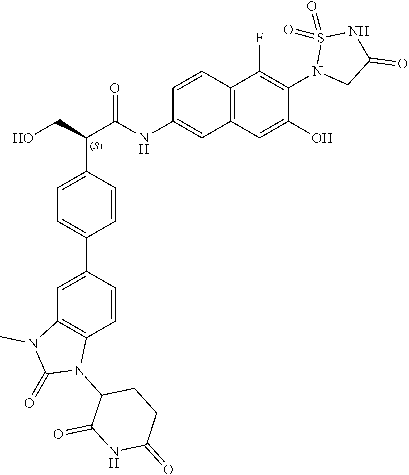 |
| 139 | 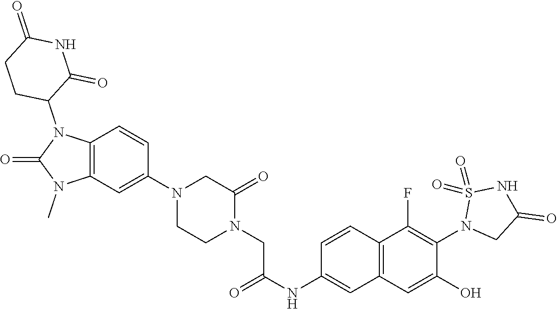 [SMILES: Cn1c(=O)n(C2CCC(=O)NC2=O)c2ccc(N3CCN(CC(=O)Nc4ccc5c(F)c(N6CC(=O)NS6(=O)=O)c(O)cc5c4)C(=O)C3)cc21] |
| 140 | 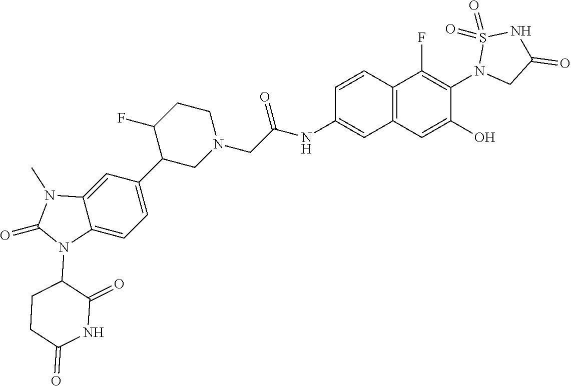 [SMILES: Cn1c(=O)n(C2CCC(=O)NC2=O)c2ccc(C3CN(CC(=O)Nc4ccc5c(F)c(N6CC(=O)NS6(=O)=O)c(O)cc5c4)CCC3F)cc21] |
| 141 | 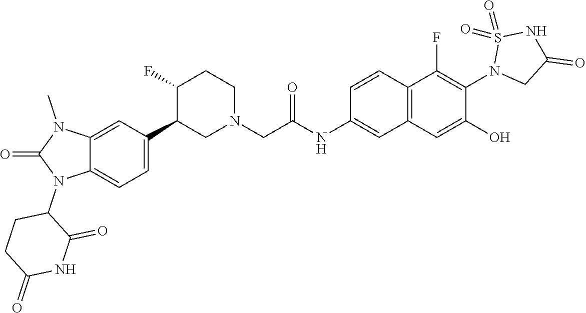 [SMILES: Cn1c(=O)n(C2CCC(=O)NC2=O)c2ccc([C@@H]3CN(CC(=O)Nc4ccc5c(F)c(N6CC(=O)NS6(=O)=O)c(O)cc5c4)CC[C@H]3F)cc21] |
| 142 | 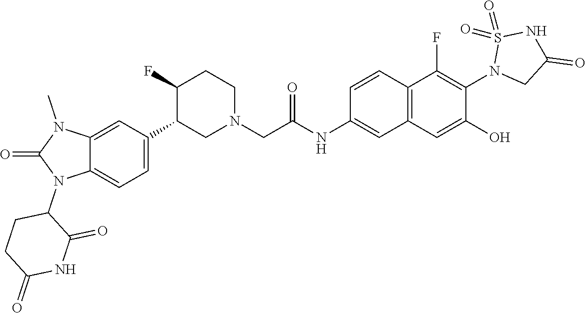 [SMILES: Cn1c(=O)n(C2CCC(=O)NC2=O)c2ccc([C@H]3CN(CC(=O)Nc4ccc5c(F)c(N6CC(=O)NS6(=O)=O)c(O)cc5c4)CC[C@@H]3F)cc21] |
| 143 | 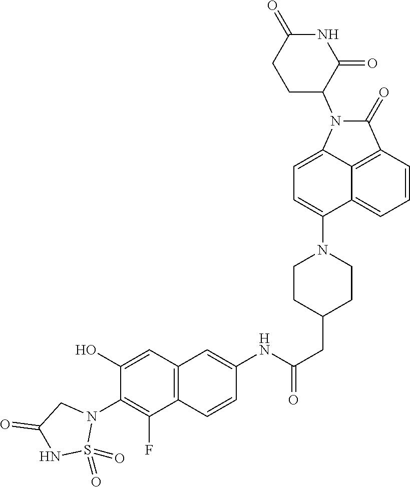 [SMILES: O=C1CCC(N2C(=O)c3cccc4c(N5CCC(CC(=O)Nc6ccc7c(F)c(N8CC(=O)NS8(=O)=O)c(O)cc7c6)CC5)ccc2c34)C(=O)N1] |
| 144 | 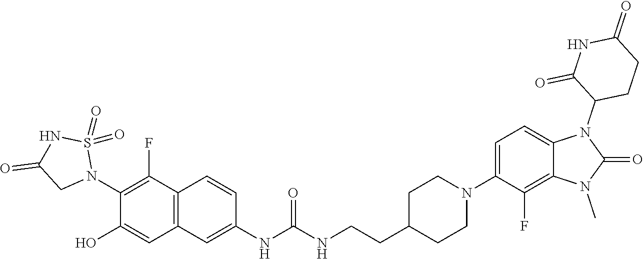 [SMILES: Cn1c(=O)n(C2CCC(=O)NC2=O)c2ccc(N3CCC(CCNC(=O)Nc4ccc5c(F)c(N6CC(=O)NS6(=O)=O)c(O)cc5c4)CC3)c(F)c21] |
| 145 | 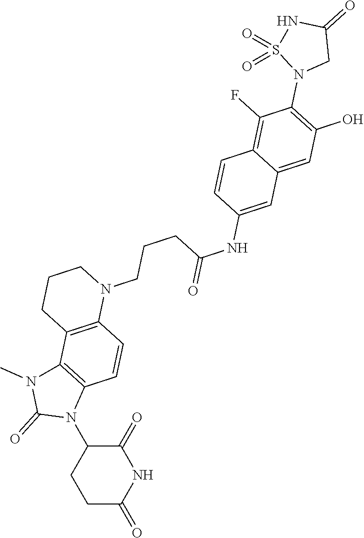 [SMILES: Cn1c(=O)n(C2CCC(=O)NC2=O)c2ccc3c(c21)CCCN3CCCC(=O)Nc1ccc2c(F)c(N3CC(=O)NS3(=O)=O)c(O)cc2c1] |
| 146 | 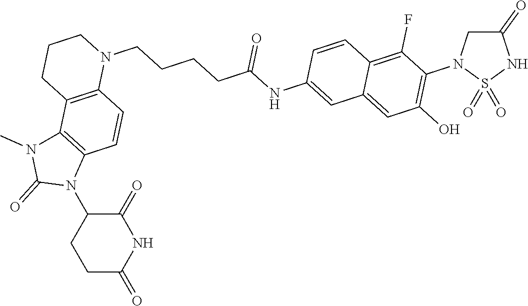 [SMILES: Cn1c(=O)n(C2CCC(=O)NC2=O)c2ccc3c(c21)CCCN3CCCCC(=O)Nc1ccc2c(F)c(N3CC(=O)NS3(=O)=O)c(O)cc2c1] |
| 147 | 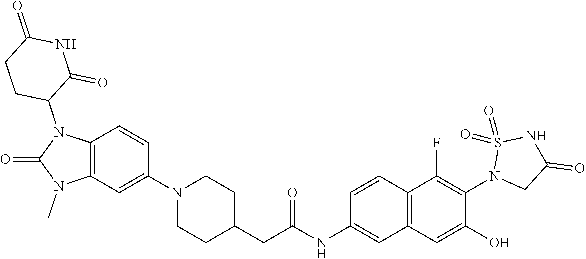 [SMILES: Cn1c(=O)n(C2CCC(=O)NC2=O)c2ccc(N3CCC(CC(=O)Nc4ccc5c(F)c(N6CC(=O)NS6(=O)=O)c(O)cc5c4)CC3)cc21] |
| 148 | 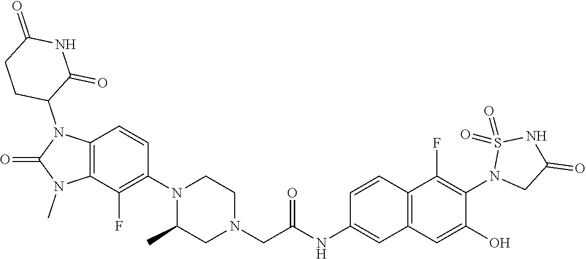 [SMILES: C[C@@H]1CN(CC(=O)Nc2ccc3c(F)c(N4CC(=O)NS4(=O)=O)c(O)cc3c2)CCN1c1ccc2c(c1F)n(C)c(=O)n2C1CCC(=O)NC1=O] |
| 149 | 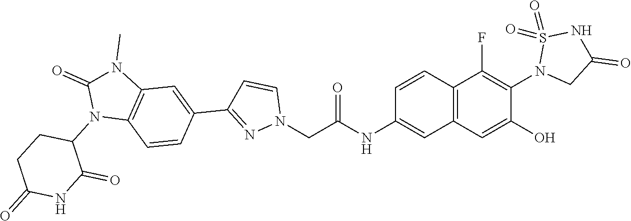 [SMILES: Cn1c(=O)n(C2CCC(=O)NC2=O)c2ccc(-c3ccn(CC(=O)Nc4ccc5c(F)c(N6CC(=O)NS6(=O)=O)c(O)cc5c4)n3)cc21] |
| 150 | 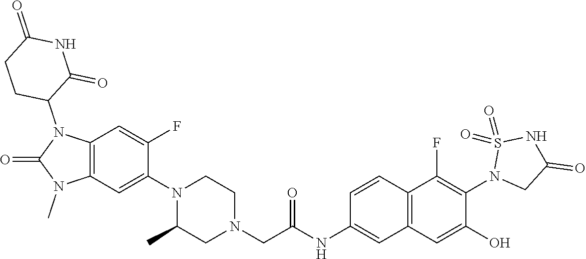 [SMILES: C[C@@H]1CN(CC(=O)Nc2ccc3c(F)c(N4CC(=O)NS4(=O)=O)c(O)cc3c2)CCN1c1cc2c(cc1F)n(C1CCC(=O)NC1=O)c(=O)n2C] |
| 151 | 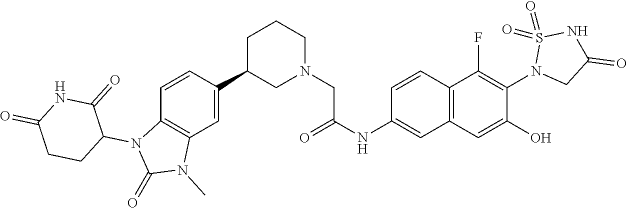 [SMILES: Cn1c(=O)n(C2CCC(=O)NC2=O)c2ccc([C@H]3CCCN(CC(=O)Nc4ccc5c(F)c(N6CC(=O)NS6(=O)=O)c(O)cc5c4)C3)cc21] |
| 152 | 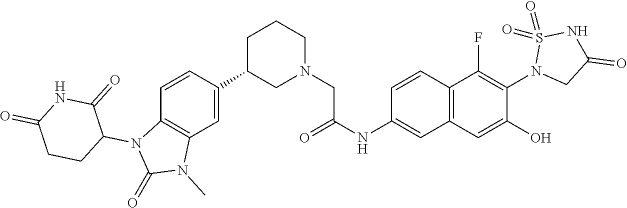 [SMILES: Cn1c(=O)n(C2CCC(=O)NC2=O)c2ccc([C@@H]3CCCN(CC(=O)Nc4ccc5c(F)c(N6CC(=O)NS6(=O)=O)c(O)cc5c4)C3)cc21] |
| 153 | 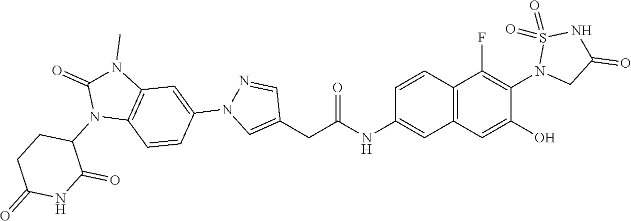 [SMILES: Cn1c(=O)n(C2CCC(=O)NC2=O)c2ccc(-n3cc(CC(=O)Nc4ccc5c(F)c(N6CC(=O)NS6(=O)=O)c(O)cc5c4)cn3)cc21] |
| 154 | 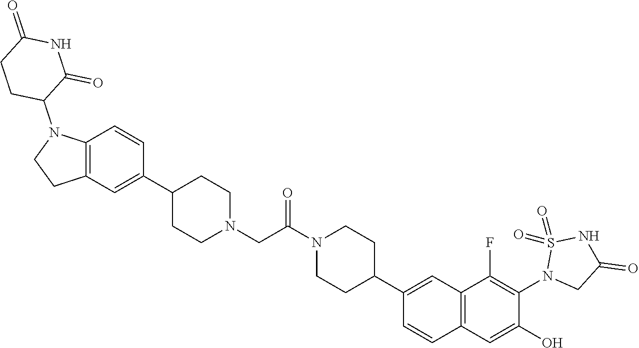 [SMILES: O=C1CCC(N2CCc3cc(C4CCN(CC(=O)N5CCC(c6ccc7cc(O)c(N8CC(=O)NS8(=O)=O)c(F)c7c6)CC5)CC4)ccc32)C(=O)N1] |
| 155 | 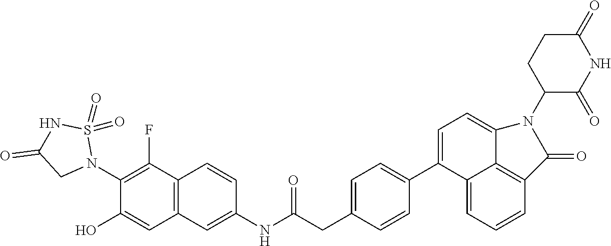 [SMILES: O=C1CCC(N2C(=O)c3cccc4c(-c5ccc(CC(=O)Nc6ccc7c(F)c(N8CC(=O)NS8(=O)=O)c(O)cc7c6)cc5)ccc2c34)C(=O)N1] |
| 156 | 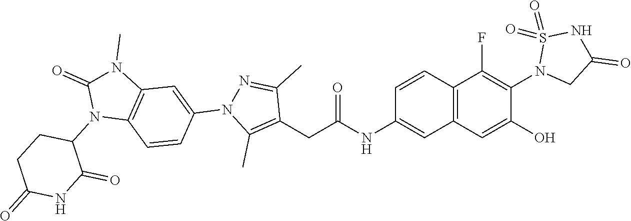 [SMILES: Cc1nn(-c2ccc3c(c2)n(C)c(=O)n3C2CCC(=O)NC2=O)c(C)c1CC(=O)Nc1ccc2c(F)c(N3CC(=O)NS3(=O)=O)c(O)cc2c1] |
| 157 | 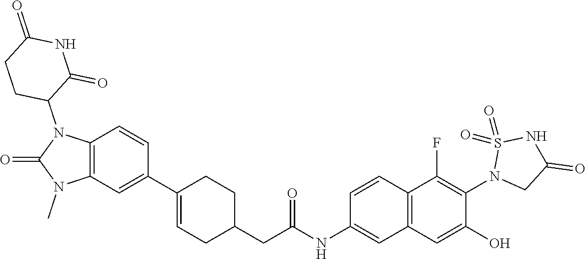 [SMILES: Cn1c(=O)n(C2CCC(=O)NC2=O)c2ccc(C3=CCC(CC(=O)Nc4ccc5c(F)c(N6CC(=O)NS6(=O)=O)c(O)cc5c4)CC3)cc21] |

Some embodiments provide a compound of Formula (III):

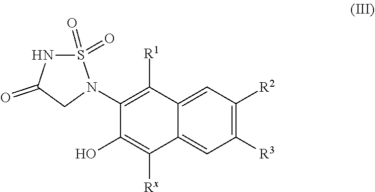

or a pharmaceutically acceptable salt thereof, wherein:
R¹ is hydrogen or halogen;
R² is hydrogen, halogen, C1-C3 alkoxy, C3-C6 cycloalkoxy, C1-C3 haloalkoxy, C3-C5 halocycloalkoxy, C1-C3 alkyl, C1-C3 haloalkyl, C3-C6 cycloalkyl, or -L-Q¹;
R³ is hydrogen, halogen, C1-C3 alkoxy, C3-C5 cycloalkoxy, C1-C3 haloalkoxy, C3-C5 halocycloalkoxy, C1-C3 alkyl, C1-C3 haloalkyl, C3-C5 cycloalkyl, or -L-Q¹;
wherein one of R² and R³ is -L-Q¹ and the other of R² and R³ is not -L-Q¹;
R^{x} is hydrogen or halogen;
L is —U—V—W—X—Y—;
U is a bond, —(NR⁴)—, —O—, C1-C3 alkylene, C2-C3 alkenylene, C2-C3 alkynylene, C3-C6 cycloalkylene, 4-10 membered heterocyclylene, 5-10 membered heteroarylene, —(C═O)NR⁴—, —NR⁴(C═O)—, —OR⁵—, —R⁵O—, —NR⁴R⁵—, —R⁵NR⁴—, or —(NR⁴)(C═O)(NR⁴)—;
each R⁴ is independently a hydrogen, C1-C6 alkyl, or C3-C5 cycloalkyl;
R⁵ is C1-C3 alkylene, C3-C7 cycloalkylene, or 4-12 membered heterocyclylene;
V is a bond, —(NR⁴)—, —O—, C1-C6 alkylene, C2-C6 alkenylene, —(C═O)NR⁴—, —(NR⁴)R⁵—, —(NR⁴)(C═O)—, —NH(C═O)NH—, —OR⁵—, —R⁵O—, 4-10-membered heterocyclylene, 5-10 membered heteroarylene, C6-C10 arylene, or C3-C6 cycloalkylene;
W is a bond, C1-C3 alkylene optionally substituted with hydroxyl, C3-C6 cycloalkylene, 4-12 membered heterocyclylene, —O—, —(NR⁴)—, —R⁵(NR⁴)—, —(NR⁴)R⁵—, —(NR⁴)(C═O)—, —R⁵(NR⁴)(C═O)—, —(C═O)(NR⁴)R⁵—, —R⁵(C═O)(NR⁴)—, —(C═O)(NR⁴)—, —R⁵(C═O)—, —(C═O)R⁵—, —(C═O)—, —(S═O)—, or —S(O₂)—;
X is a bond, C1-C3 alkylene, C3-C6 cycloalkylene, 4-12 membered heterocyclylene, C6-C10 arylene, 5-10 membered heteroarylene, —R⁵(NR⁴)(C═O)—, —(C═O)R⁵(NR⁴)—, —R⁵(C═O)(NR⁴)—, —(NR⁴)(C═O)R⁵—, —R⁵(C═O)(NR⁴)—, —(C═O)(NR⁴)R⁵—, —(NR⁴)R⁵(C═O)—, —R⁵(C═O)(NR⁴)R⁵—, —R⁵(NR⁴)(C═O)R⁵—, —(C═O)R⁵—, or —R⁵(C═O)—;
Y is R⁶, R⁶(CR^{A}R^{B})ₚ-Q-, or -Q-(CR^{A}R^{B})ₚR⁶—;
Q is selected from the group consisting of —(NR⁴)—, —O—, and —(CR^{A}R^{B})ₚ;
p is 0, 1, 2, or 3;
R⁶ is C1-C3 alkylene, C3-C7 cycloalkylene, 4-12 membered heterocyclylene, C6-C10 arylene, or 5-10 membered heteroarylene;
wherein the heterocyclylene, heteroarylene, arylene, and cycloalkylene groups of U, V, W, X, and R⁶ are each optionally substituted with 1-3 substituents independently selected from fluoro, hydroxyl, C1-C6 alkoxy, and C1-C6 alkyl;
each R^{A} and R^{B} is independently hydrogen, fluoro, or C1-C6 alkyl; or
R^{A} and R^{B}, together with the carbon atom to which they are attached, come together to form a C3-C4 cycloalkyl; or
R^{A} and R^{B} combine to form oxo; and
Q¹ is —NH₂, —OH, —CO₂H, —(C═O)C1, —N₃, or C2-C6 alkyne.

### Pharmaceutical Compositions

Some embodiments provide a pharmaceutical composition comprising a compound of Formula (I), or a pharmaceutically acceptable salt thereof.

### Methods of Treatment

The present disclosure features compounds, compositions, and methods comprising a compound of Formula (I). In some embodiments, the compounds, compositions, and methods described herein are used in the prevention or treatment of a disease. Exemplary diseases include, but are not limited to cancer, type-2 diabetes, metabolic syndrome, obesity, NAFLD, NASH, or another metabolic disease.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

### Synthetic Protocols

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures. General scheme relating to methods of making exemplary compounds of the disclosure are additionally described herein.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

### Abbreviations

APCI for atmospheric pressure chemical ionization; DCI for desorption chemical ionization; DMSO for dimethyl sulfoxide; ESI for electrospray ionization; HPLC for high performance liquid chromatography; LC/MS for liquid chromatography/mass spectrometry; LED for light-emitting diode; MS for mass spectrum; NMR for nuclear magnetic resonance; psi for pounds per square inch; and TLC for thin-layer chromatography.

### Examples

### Preparation of Exemplary Intermediates

### 5-(3-(Benzyloxy)-7-bromo-1-fluoronaphthalen-2-yl)-1,2,5-thiadiazolidin-3-one 1,1-dioxide (12)

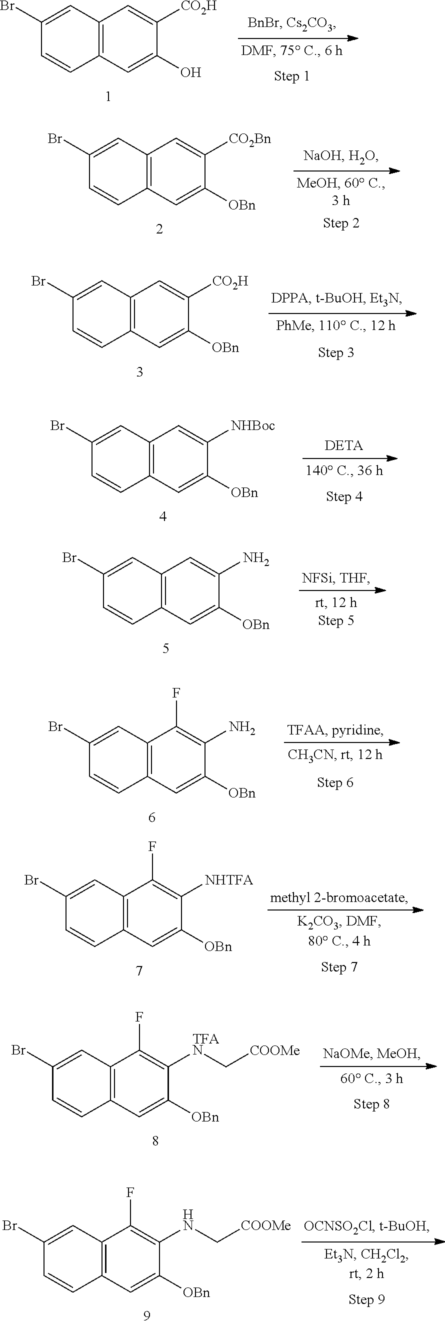

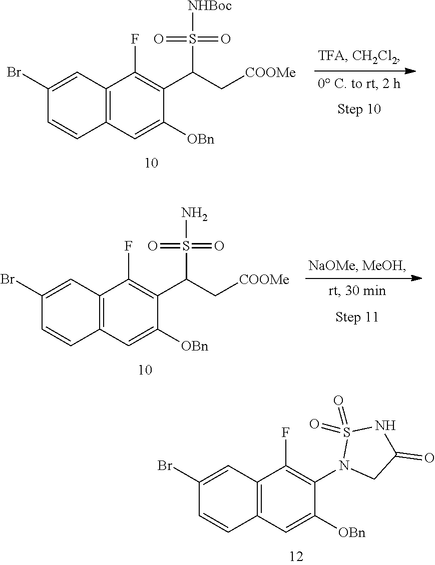 [SMILES: COC(=O)CC(c1c(OCc2ccccc2)cc2ccc(Br)cc2c1F)S(=O)(=O)NC(=O)OC(C)(C)C ; COC(=O)CC(c1c(OCc2ccccc2)cc2ccc(Br)cc2c1F)S(N)(=O)=O ; O=C1CN(c2c(OCc3ccccc3)cc3ccc(Br)cc3c2F)S(=O)(=O)N1]

### Step 1: Benzyl 3-(benzyloxy)-7-bromo-2-naphthoate (2)

A 100 mL round-bottom flask was charged with 7-bromo-3-hydroxy-2-naphthoic acid (1, [1779-11-9], 5 g, 18.7 mmol) and cesium carbonate (18.30 g, 56.2 mmol), followed by DMF (35 mL). The mixture was rapidly stirred to suspend the reaction components, followed by treatment with benzyl bromide (4.45 mL, 37.4 mmol) at room temperature. After 2 h, the mixture was poured into water (70 mL), and the resulting white solid precipitate collected by filtration. The solid thus obtained was washed with water (3×50 mL), triturated with 30% methyl tert-butylmethyl ether/petroleum ether (20 mL), filtered, and dried under vacuum to afford benzyl 3-(benzyloxy)-7-bromo-2-naphthoate (2, 8 g, 17.2 mmol, 92% yield) as a white solid. LCMS (TFA, ESI+): m/z 447.1 [M+H]^{+ 1}H NMR (400 MHz, DMSO-d₆) δ 5.27 (s, 2H), 5.35 (s, 2H), 7.30-7.45 (m, 8H), 7.49 (d, J=6.8 Hz, 2H), 7.60-7.71 (m, 2H), 7.82 (d, J=8.8 Hz, 1H), 8.28 (d, J=1.5 Hz, 1H), 8.32 (s, 1H).

### Step 2: 3-(Benzyloxy)-7-bromo-2-naphthoic acid (3)

To a solution of benzyl 3-(benzyloxy)-7-bromo-2-naphthoate (2, 4 g, 8.5 mmol) in MeOH (60 mL) and water (30.0 mL) at room temperature was added LiOH (0.407 g, 17.0 mmol). The mixture was heated to 70° C. for 2 h and was then concentrated. The resulting residue was diluted with water (500 mL). The aqueous layer was acidified with 1 M HCl to pH=3, and the solid was filtered and dried under vacuum to give 3-(benzyloxy)-7-bromo-2-naphthoic acid (3, 3 g, 8.0 mmol, 94% yield) as white solid. LCMS (TFA, ESI+): m/z 357.0 [M+H]^{+ 1}H NMR (400 MHz, DMSO-d₆) δ 5.29 (s, 2H), 7.29-7.45 (m, 3H), 7.54 (d, J=7.28 Hz, 2H), 7.60 (s, 1H), 7.66 (dd, J=8.8, 2.0 Hz, 1H), 7.81 (d, J=8.8 Hz, 1H), 8.20-8.27 (m, 2H), 13.06 (br s, 1H).

### Step 3: tert-Butyl (3-(benzyloxy)-7-bromonaphthalen-2-yl)carbamate (4)

A three-neck 250 mL round bottom flask was charged with 3-(benzyloxy)-7-bromo-2-naphthoic acid (3, 6 g, 16.8 mmol), toluene (48 mL), t-BuOH (48 mL) and triethylamine (2.48 mL, 17.8 mmol). Diphenyl phosphorazidate (4.90 g, 17.8 mmol) was then added and the reaction mixture heated at 110° C. for 4 h. The solution was cooled to room temperature and concentrated to give a crude solid. The solid was triturated with ethanol (50 mL), filtered, rinsed with ethanol (10 mL), and dried under vacuum to give tert-butyl (3-(benzyloxy)-7-bromonaphthalen-2-yl)carbamate (4, 6.6 g, 13.9 mmol, 83% yield) as white solid. LCMS (NH₄HCO₃, ESI−): m/z 426.1 [M−H]^{- 1}H NMR (400 MHz, DMSO-d₆) δ 1.48 (s, 9H), 5.29 (s, 2H), 7.34-7.50 (m, 5H), 7.57 (d, J=7.0 Hz, 2H), 7.68 (d, J=8.8 Hz, 1H), 8.02 (d, J=1.7 Hz, 1H), 8.13 (s, 1H), 8.21 (s, 1H).

### Step 4: 3-(Benzyloxy)-7-bromonaphthalen-2-amine (5)

To a solution of tert-butyl (3-(benzyloxy)-7-bromonaphthalen-2-yl)carbamate (4, 8 g, 86% purity, 16 mmol) was added diethylenetriamine (26.2 g, 254 mmol) and the mixture was stirred at 130° C. for 3 h. The reaction was cooled to room temperature, and water (50 mL) was added to the mixture and stirred 10 min. The solid was filtered and the filter cake was washed with 10 mL of i-PrOH and dried under vacuum to give 3-(benzyloxy)-7-bromonaphthalen-2-amine (5, 4.5 g, 12.3 mmol, 78% yield) as pink solid. LCMS (TFA, ESI+): m/z 328.1 [M+H]^{+ 1}H NMR (400 MHz, DMSO-d₆) δ 5.25 (s, 2H), 5.37 (s, 2H), 6.89 (s, 1H), 7.18 (dd, J=8.6, 2.0 Hz, 1H), 7.27-7.37 (m, 2H), 7.38-7.45 (m, 2H), 7.54 (t, J=7.7 Hz, 3H), 7.70 (d, J=1.7 Hz, 1H).

### Step 5: 3-(Benzyloxy)-7-bromo-1-fluoronaphthalen-2-amine (6)

To a solution of 3-(benzyloxy)-7-bromonaphthalen-2-amine (5, 20 g, 90% purity, 54.8 mmol) in THF (100 mL) was added a solution of N-fluorobenzenesulfonimide (19.0 g, 60.3 mmol) in THF (100 mL) at 0° C. over the period of 1 h. The mixture was warmed to room temperature and stirred for an additional 1 h. Then residual oxidant was quenched by adding a solution of sodium thiosulfate pentahydrate (17.3 g, 110 mmol) in water (100 mL), and the mixture stirred at room temperature for 20 min. The aqueous phase was extracted with ethyl acetate (3×100 mL) and the combined organic phases washed with brine (2×100 mL); then dried over Na₂SO₄, filtered and concentrated under reduced pressure. The residue was purified by silica gel chromatography (0% to 10% ethyl acetate:petroleum ether) to give 3-(benzyloxy)-7-bromo-1-fluoronaphthalen-2-amine (6, 8 g, 20.8 mmol, 38% yield) as yellow solid. LCMS (TFA, ESI+): m/z 346.2 [M+H]^{+ 1}H NMR (400 MHz, DMSO-d₆) δ 5.28 (s, 2H), 5.31 (s, 2H), 7.25 (s, 1H), 7.30-7.36 (m, 2H), 7.39-7.44 (m, 2H), 7.56 (br d, J=7.1 Hz, 2H), 7.65 (dd, J=8.6, 1.3 Hz, 1H), 7.82 (d, J=1.6 Hz, 1H).

### Step 6: N-(3-(Benzyloxy)-7-bromo-1-fluoronaphthalen-2-yl)-2,2,2-trifluoroacetamide (7)

To a solution of 3-(benzyloxy)-7-bromo-1-fluoronaphthalen-2-amine (6, 2 g, 90% purity, 5.2 mmol) in acetonitrile (40 mL) and pyridine (1.3 mL, 15.6 mmol) at 0° C. was added trifluoroacetic anhydride (1.49 mL, 10.4 mmol), and the mixture allowed to warm slowly to room temperature. After 2 h, the mixture was diluted with water (20 mL) and extracted with ethyl acetate (3×20 mL). The organic layers were washed with brine (20 mL), dried with Na₂SO₄, filtered, and concentrated under reduced pressure to give N-(3-(benzyloxy)-7-bromo-1-fluoronaphthalen-2-yl)-2,2,2-trifluoroacetamide (7, 2.5 g, 4.8 mmol, 92% yield) as an off-white solid which was used in the next step directly. LCMS (TFA, ESI+): m/z 442.0 [M+H]^{+ 1}H NMR (400 MHz, DMSO-d₆) δ 5.28 (s, 2H), 7.30-7.35 (m, 1H), 7.39 (t, J=7.3 Hz, 2H), 7.46 (br d, J=7.0 Hz, 2H), 7.53 (s, 1H), 7.70-7.75 (m, 1H), 7.88 (d, J=8.4 Hz, 1H), 8.15 (s, 1H).

### Step 7: Methyl 2-(N-(3-(benzyloxy)-7-bromo-1-fluoronaphthalen-2-yl)-2,2,2-trifluoroacetamido)acetate (8)

To a solution of N-(3-(benzyloxy)-7-bromo-1-fluoronaphthalen-2-yl)-2,2,2-trifluoroacetamide (7, 2.5 g, 85% purity, 4.81 mmol) in DMF (30 mL) was added K₂CO₃ (1.33 g, 9.61 mmol) and methyl 2-bromoacetate (1.10 g, 7.21 mmol). The reaction was heated to 80° C. and stirred for 1 h. The mixture was cooled to room temperature and diluted with water (30 mL). The aqueous mixture was extracted with ethyl acetate (3×20 mL), and the combined organic phases were washed with brine (3×20 mL), dried with Na₂SO₄, filtered and concentrated under reduced pressure to give methyl 2-(N-(3-(benzyloxy)-7-bromo-1-fluoronaphthalen-2-yl)-2,2,2-trifluoroacetamido)acetate (8, 3.4 g, 5.95 mmol, 93% yield) as an off-white solid which was used in next step directly. LCMS (TFA, ESI+): m/z 514.0 [M+H]^{+ 1}H NMR (400 MHz, DMSO-d₆) δ 8.27 (d, J=2.0 Hz, 1H), 8.11 (d, J=9.0 Hz, 1H), 7.78-7.66 (m, 2H), 7.51-7.33 (m, 5H), 5.27 (q, J=11.9 Hz, 2H), 4.45 (d, J=1.7 Hz, 2H), 3.59 (s, 3H).

### Step 8: Methyl 2-((3-(benzyloxy)-7-bromo-1-fluoronaphthalen-2-yl)amino)acetate (9)

To a solution of methyl 2-(N-(3-(benzyloxy)-7-bromo-1-fluoronaphthalen-2-yl)-2,2,2-trifluoroacetamido)acetate (8, 3.4 g, 85% purity, 5.6 mmol) in MeOH (40 mL) was added sodium methoxide (4.29 g, 23.8 mmol) at room temperature. The mixture was heated to 60° C. and stirred for 3 h. Upon completion, the mixture was cooled to room temperature, diluted with water (30 mL), and the aqueous mixture extracted with ethyl acetate (3×20 mL). The combined organic phases were washed with brine (2×20 mL), dried with Na₂SO₄, filtered, and concentrated under reduced pressure to give methyl 2-((3-(benzyloxy)-7-bromo-1-fluoronaphthalen-2-yl)amino)acetate (9, 1.9 g, 4.1 mmol, 69% yield) as an off-white solid which was used directly in the next step. LCMS (TFA, ESI+): m/z 418.2 [M+H]¹H NMR (400 MHz, DMSO-d₆) δ 3.63 (s, 3H), 4.22 (dd, J=6.7, 4.0 Hz, 2H), 5.30 (s, 2H), 7.30 (s, 1H), 7.34-7.39 (m, 2H), 7.41-7.45 (m, 2H), 7.55 (d, J=7.1 Hz, 2H), 7.67 (dd, J=8.7, 1.5 Hz, 1H), 7.80 (d, J=1.7 Hz, 1H).

### Step 9: Methyl 2-((3-(benzyloxy)-7-bromo-1-fluoronaphthalen-2-yl)(N-(tert-butoxycarbonyl)sulfamoyl)amino)acetate (10)

To a solution of sulfurisocyanatidic chloride (1.22 g, 8.61 mmol) in CH₂Cl₂ (10 mL) was added a solution of t-BuOH (1.30 g, 17.5 mmol) in CH₂Cl₂ (10 mL), dropwise, at 0° C. The mixture was warmed to room temperature and stirred for an additional 1 h. After cooling to 0° C., a solution of triethylamine (2.40 mL, 17.2 mmol) and methyl 2-((3-(benzyloxy)-7-bromo-1-fluoronaphthalen-2-yl)amino)acetate (9, 2 g, 90% purity, 4.30 mmol) in CH₂Cl₂ (20 mL) was slowly added to the reaction mixture. Upon complete addition, the solution was warmed to room temperature and stirred for 2 h. The mixture was concentrated under pressure to give methyl 2-((3-(benzyloxy)-7-bromo-1-fluoronaphthalen-2-yl)(N-(tert-butoxycarbonyl)sulfamoyl)amino)acetate (10, 5 g, 6.70 mmol, 89% yield) as yellow oil. The crude product was used for the next step without purification. LCMS (TFA, ESI+): m/z 497.2 [M−Boc+H]^{+ 1}H NMR (400 MHz, DMSO-d₆) δ 11.40 (s, 1H), 8.15 (d, J=2.0 Hz, 1H), 7.83 (dd, J=8.9, 1.3 Hz, 1H), 7.71 (dd, J=8.9, 2.0 Hz, 1H), 7.60-7.48 (m, 2H), 7.47-7.30 (m, 4H), 5.31 (q, J=12.8 Hz, 2H), 4.75 (d, J=17.9 Hz, 1H), 4.48 (d, J=17.9 Hz, 1H), 3.56 (s, 3H), 1.32 (s, 9H).

### Step 10: Methyl 2-((3-(benzyloxy)-7-bromo-1-fluoronaphthalen-2-yl)(sulfamoyl)amino)acetate (11)

To a solution of methyl 2-((3-(benzyloxy)-7-bromo-1-fluoronaphthalen-2-yl)(N-(tert-butoxycarbonyl)sulfamoyl)amino)acetate (10, 15 g, 75% purity, 18.8 mmol) in CH₂Cl₂ (100 mL) at 0° C. was added 2,2,2-trifluoroacetic acid (35 mL, 18.8 mmol), then warmed to room temperature and stirred for 1 h. The mixture was concentrated under reduced pressure, and the residue diluted with water (300 mL). The aqueous mixture was made basic by addition of solid NaHCO₃ (pH=8) and then extracted with ethyl acetate (3×150 mL). The combined organic layers were dried over Na₂SO₄, filtered, and concentrated under reduced pressure to give methyl 2-((3-(benzyloxy)-7-bromo-1-fluoronaphthalen-2-yl)(sulfamoyl)amino)acetate (11, 12 g, 16.9 mmol, 90% yield) as white solid which was used for next step without further purification. LCMS (TFA, ESI+): m/z 496.9 [M+H]^{+ 1}H NMR (400 MHz, DMSO-d₆) δ 3.56 (s, 3H), 4.29-4.36 (m, 1H), 4.46-4.53 (m, 1H), 5.27 (s, 2H), 7.11 (s, 2H), 7.39-7.46 (m, 4H), 7.58 (d, J=7.2 Hz, 2H), 7.69 (dd, J=8.8, 2.0 Hz, 1H), 7.81-7.86 (m, 1H), 8.13 (d, J=2.0 Hz, 1H).

### Step 11: 5-(3-(Benzyloxy)-7-bromo-1-fluoronaphthalen-2-yl)-1,2,5-thiadiazolidin-3-one 1,1-dioxide (12)

To a solution of methyl 2-((3-(benzyloxy)-7-bromo-1-fluoronaphthalen-2-yl)(sulfamoyl)amino)acetate (11, 9 g, 85% purity, 15.4 mmol) in THF (100 mL) at room temperature was added solution of 30% sodium methoxide in methanol (29.3 mL, 8.31 g, 46.1 mmol) and stirring was continued for 1 h. The reaction was concentrated, taken up in water (10 mL), and acidified with 1 M HCl (pH=5). The aqueous mixture was extracted with ethyl acetate (3×20 mL). The combined organic phases were dried over anhydrous Na₂SO₄, filtered, and concentrated to give 5-(3-(benzyloxy)-7-bromo-1-fluoronaphthalen-2-yl)-1,2,5-thiadiazolidin-3-one 1,1-dioxide (12, 9 g, 17.4 mmol, 90% yield) as light brown solid. LCMS (NH₄HCO₃, ESI−): m/z 463.0 [M−H]^{- 1}H NMR (400 MHz, DMSO-d₆) δ 4.53 (s, 2H), 5.28 (s, 2H), 7.30-7.43 (m, 4H), 7.52 (br d, J=7.6 Hz, 3H), 7.74 (dd, J=8.8, 1.8 Hz, 1H), 7.87 (d, J=8.8 Hz, 1H), 8.16 (d, J=1.4 Hz, 1H).

### 5-(6-amino-3-(benzyloxy)-1-fluoronaphthalen-2-yl)-1,2,5-thiadiazolidin-3-one 1,1-dioxide (2)

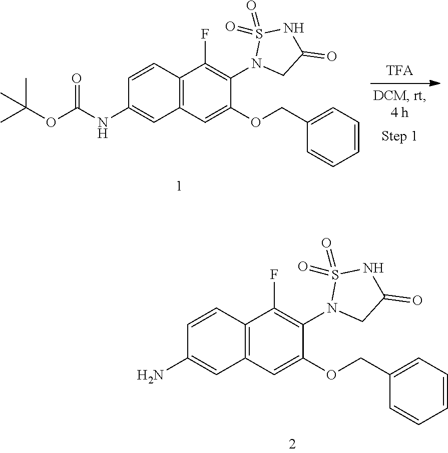 [SMILES: CC(C)(C)OC(=O)Nc1ccc2c(F)c(N3CC(=O)NS3(=O)=O)c(OCc3ccccc3)cc2c1 ; Nc1ccc2c(F)c(N3CC(=O)NS3(=O)=O)c(OCc3ccccc3)cc2c1]

### Step 1: 5-(6-amino-3-(benzyloxy)-1-fluoronaphthalen-2-yl)-1,2,5-thiadiazolidin-3-one 1,1-dioxide (2)

A stirred solution of tert-butyl N-[7-benzyloxy-5-fluoro-6-(1,1,4-trioxo-1,2,5-thiadiazolidin-2-yl)-2-naphthyl]carbamate (1, 1.0 g, 1.99 mmol) in DCM (10 mL) at 0° C. was treated with trifluoroacetic acid (227.35 mg, 1.99 mmol, 153.62 μL) via dropwise addition. The reaction mixture was stirred at room temperature for 4 h. The reaction mixture was concentrated under reduced pressure, azeotroped with toluene, and triturated with diethyl ether to obtain 5-(6-amino-3-benzyloxy-1-fluoro-2-naphthyl)-1,1-dioxo-1,2,5-thiadiazolidin-3-one (2, 850 mg, 1.58 mmol, 79% yield, TFA salt) as an off-white solid. LCMS (ES+): m/z 402.1 [M+H]⁺

### 5-(6-Amino-1-fluoro-3-hydroxynaphthalen-2-yl)-1,2,5-thiadiazolidin-3-one 1,1-dioxide (6)

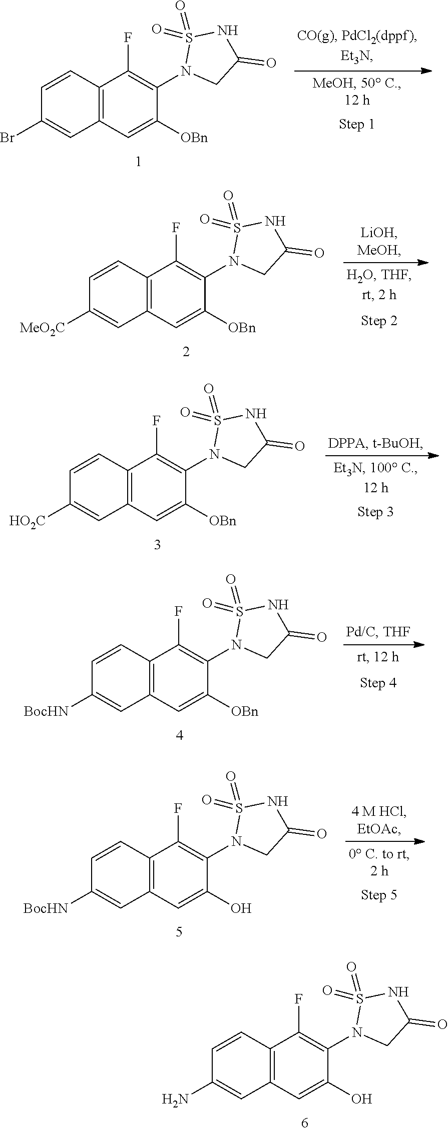 [SMILES: O=C1CN(c2c(OCc3ccccc3)cc3cc(Br)ccc3c2F)S(=O)(=O)N1 ; COC(=O)c1ccc2c(F)c(N3CC(=O)NS3(=O)=O)c(OCc3ccccc3)cc2c1 ; O=C1CN(c2c(OCc3ccccc3)cc3cc(C(=O)O)ccc3c2F)S(=O)(=O)N1 ; CC(C)(C)OC(=O)Nc1ccc2c(F)c(N3CC(=O)NS3(=O)=O)c(OCc3ccccc3)cc2c1 ; CC(C)(C)OC(=O)Nc1ccc2c(F)c(N3CC(=O)NS3(=O)=O)c(O)cc2c1 ; Nc1ccc2c(F)c(N3CC(=O)NS3(=O)=O)c(O)cc2c1]

### Step 1: Methyl 7-(benzyloxy)-6-(1,1-dioxido-4-oxo-1,2,5-thiadiazolidin-2-yl)-5-fluoro-2-naphthoate (2)

To a solution of 5-(3-(benzyloxy)-6-bromo-1-fluoronaphthalen-2-yl)-1,2,5-thiadiazolidin-3-one 1,1-dioxide (40 g, 90% purity, 77 mmol) in MeOH (400 mL) was added triethylamine (32.4 mL, 232 mmol) and [1,1′-bis(diphenylphosphino)ferrocene]dichloropalladium(II) (17.0 g, 23.2 mmol) and the reaction placed under an atmosphere of carbon monoxide (40 psi). The solution was heated to 50° C. and stirred for 12 h. The solvent was removed under reduced pressure to give methyl 7-(benzyloxy)-6-(1,1-dioxido-4-oxo-1,2,5-thiadiazolidin-2-yl)-5-fluoro-2-naphthoate (70 g, 95 mmol, 81% yield) as red solid which was used directly in the next step. LCMS (NH₄HCO₃, ESI−): m/z 443.1 [M−H]^{- 1}H NMR (400 MHz, DMSO-d₆) δ 3.92 (s, 3H), 3.97-4.27 (m, 2H), 5.28 (s, 2H), 7.26-7.41 (m, 3H), 7.45-7.68 (m, 3H), 7.78 (s, 1H), 7.84-8.02 (m, 1H), 8.23 (d, J=8.8 Hz, 1H), 8.55 (d, J=1.3 Hz, 1H).

### Step 2: 7-(Benzyloxy)-6-(1,1-dioxido-4-oxo-1,2,5-thiadiazolidin-2-yl)-5-fluoro-2-naphthoic acid (3)

To a solution of methyl 7-(benzyloxy)-6-(1,1-dioxido-4-oxo-1,2,5-thiadiazolidin-2-yl)-5-fluoro-2-naphthoate (8 g, 80% purity, 14.4 mmol) in THF (20 mL), MeOH (5 mL) and water (5 mL) was added LiOH (0.345 g, 14.4 mmol) at 0° C. The mixture was warmed to room temperature and stirred for 2 h. The reaction was concentrated to remove most of the THF then diluted with water (100 mL). The aqueous phase was washed with ethyl acetate (3×100 mL) then acidified with 1 M hydrochloride acid to pH=2. The aqueous solution was extracted with ethyl acetate (3×150 mL) and the organic layers were combined and washed with brine (150 mL); then dried with Na₂SO₄, filtered and concentrated under reduced pressure to give 7-(benzyloxy)-6-(1,1-dioxido-4-oxo-1,2,5-thiadiazolidin-2-yl)-5-fluoro-2-naphthoic acid (6.8 g, 13 mmol, 88% yield) as a yellow solid. LCMS (NH₄HCO₃, ESI−): m/z 429.1 [M−H]^{- 1}H NMR (400 MHz, DMSO-d₆) δ 4.55 (s, 2H), 5.30 (s, 2H), 7.30-7.43 (m, 3H), 7.49-7.58 (m, 2H), 7.71 (s, 1H), 7.94 (dd, J=8.7, 1.4 Hz, 1H), 8.04-8.14 (m, 1H), 8.55 (s, 1H).

### Step 3: tert-Butyl (7-(benzyloxy)-6-(1,1-dioxido-4-oxo-1,2,5-thiadiazolidin-2-yl)-5-fluoronaphthalen-2-yl)carbamate (4)

To a solution of 7-(benzyloxy)-6-(1,1-dioxido-4-oxo-1,2,5-thiadiazolidin-2-yl)-5-fluoro-2-naphthoic acid (1.3 g, 93% purity, 2.8 mmol) in t-BuOH (50 mL) was added triethylamine (0.78 mL, 5.6 mmol) and diphenylphosphoryl azide (1.14 g, 4.17 mmol). The reaction was heated to 100° C. and stirred for 12 h. The solution was concentrated under reduced pressure and diluted with water (50 mL). The aqueous mixture was extracted with ethyl acetate (3×30 mL); and the combined organic phases were washed with brine (30 mL), dried with Na₂SO₄, filtered, and concentrated under reduced pressure to give tert-butyl (7-(benzyloxy)-6-(1,1-dioxido-4-oxo-1,2,5-thiadiazolidin-2-yl)-5-fluoronaphthalen-2-yl)carbamate (4, 1.5 g, 1.9 mmol, 70% yield) as an off-white solid. LCMS (NH₄HCO₃, ESI−): m/z 500.2 [M−H]^{- 1}H NMR (400 MHz, DMSO-d₆) δ 1.47 (s, 9H), 4.30 (s, 2H), 5.22 (s, 2H), 7.18 (s, 1H), 7.28-7.42 (m, 4H), 7.49 (d, J=7.5 Hz, 2H), 7.84 (d, J=8.8 Hz, 1H), 8.00 (s, 1H).

### Step 4: tert-Butyl (6-(1,1-dioxido-4-oxo-1,2,5-thiadiazolidin-2-yl)-5-fluoro-7-hydroxynaphthalen-2-yl)carbamate (5)

To a solution of tert-butyl (7-(benzyloxy)-6-(1,1-dioxido-4-oxo-1,2,5-thiadiazolidin-2-yl)-5-fluoronaphthalen-2-yl)carbamate (0.9 g, 90% purity, 1.6 mmol) in THF (10 mL) was added Pd/C (17 mg, 0.16 mmol) at room temperature. Stirring was continued for 12 h under a hydrogen atmosphere (15 psi). The resulting suspension was filtered through Celite and the pad washed with MeOH (75 mL). The combined filtrates were concentrated to dryness to give tert-butyl (6-(1,1-dioxido-4-oxo-1,2,5-thiadiazolidin-2-yl)-5-fluoro-7-hydroxynaphthalen-2-yl)carbamate (5, 0.8 g, 1.6 mmol, 96% yield) as white solid which was used directly in the next step. LCMS (NH₄HCO₃, ESI−): m/z 410.1 [M−H]^{- 1}H NMR (400 MHz, DMSO-d₆) δ 1.50 (s, 9H), 4.06 (s, 2H), 6.90 (s, 1H), 7.35 (dd, J=9.1, 1.8 Hz, 1H), 7.76 (d, J=8.9 Hz, 1H), 7.91 (s, 1H), 9.56-9.70 (m, 2H).

### Step 5: 5-(6-Amino-1-fluoro-3-hydroxynaphthalen-2-yl)-1,2,5-thiadiazolidin-3-one 1,1-dioxide (6)

A solution of tert-butyl (6-(1,1-dioxido-4-oxo-1,2,5-thiadiazolidin-2-yl)-5-fluoro-7-hydroxynaphthalen-2-yl)carbamate (5 g, 90% purity, 11 mmol) in ethyl acetate (30 mL) was treated with 4 M HCl (2.7 mL, 11 mmol) at 0° C. Upon completion of addition, the mixture was warmed to room temperature, and stirring was continued for 2 h. The solution was concentrated under reduced pressure to give a crude product which was purified by preparative HPLC The column used for chromatography was [column: Xbridge Shield RP18, 2.1×50 mm, 5 μm particles; detection: DAD; MS: negative electrospray ionization, range: 100-1000; mobile phase: A: 10 mM ammonium bicarbonate_{(aq)}; mobile phase B: acetonitrile; gradient: 5-95% B in 2.05 min, 5% B in 0.01 min, 5-95% B (0.01-1.00 min), 95-100% B (1.00-1.80 min), 5% B in 1.81 min with a hold at 5% B for 0.24 min; and flowrate: 1.0 mL/min]. The appropriate fractions were collected, and the sample was lyophilized to give 5-(6-amino-1-fluoro-3-hydroxynaphthalen-2-yl)-1,2,5-thiadiazolidin-3-one 1,1-dioxide (6, 3.28 g, 9.49 mmol, 87% yield, ammonium salt) as an off white solid. LCMS (NH₄HCO₃, ESI−): m/z 310.0 [M−H]^{- 1}H NMR (400 MHz, DMSO-d₆) δ 4.05 (s, 2H), 6.63 (d, J=13.6 Hz, 2H), 6.77 (dd, J=8.9, 2.0 Hz, 1H), 6.97 (s, 1H), 7.10 (s, 1H), 7.22 (s, 1H), 7.57 (d, J=8.9 Hz, 1H), 9.29 (br s, 1H).

### 7-benzyloxy-5-fluoro-6-(1,1,4-trioxo-1,2,5-thiadiazolidin-2-yl)naphthalene-2-carboxylic acid (3)

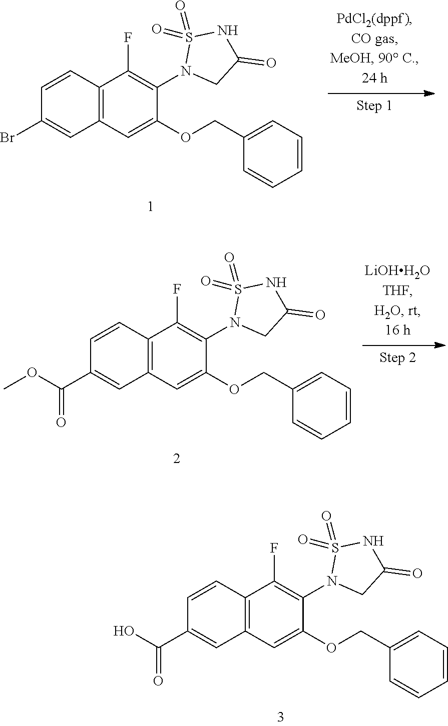 [SMILES: O=C1CN(c2c(OCc3ccccc3)cc3cc(Br)ccc3c2F)S(=O)(=O)N1 ; COC(=O)c1ccc2c(F)c(N3CC(=O)NS3(=O)=O)c(OCc3ccccc3)cc2c1 ; O=C1CN(c2c(OCc3ccccc3)cc3cc(C(=O)O)ccc3c2F)S(=O)(=O)N1]

### Step 1: methyl 7-benzyloxy-5-fluoro-6-(1,1,4-trioxo-1,2,5-thiadiazolidin-2-yl)naphthalene-2-carboxylate (2)

To a stirred solution of 5-(3-benzyloxy-6-bromo-1-fluoro-2-naphthyl)-1,1-dioxo-1,2,5-thiadiazolidin-3-one (1, 2.5 g, 5.37 mmol) in methanol (15 mL) was added triethylamine (2.72 g, 26.86 mmol, 3.74 mL) and the solution was purged with nitrogen for 10 min. Then, [1,1′-bis(diphenylphosphino)ferrocene]dichloropalladium(II) (196.43 mg, 268.65 μmol) was added. The reaction mixture was heated at 90° C. under carbon monoxide atmosphere (5 kg pressure). After 24 h, the reaction mixture was filtered through Celite and washed with methanol (30 mL). The filtrate was concentrated under reduced pressure to obtain methyl 7-benzyloxy-5-fluoro-6-(1,1,4-trioxo-1,2,5-thiadiazolidin-2-yl)naphthalene-2-carboxylate (2, 3.8 g, 5.33 mmol, 99% yield) as a brown solid. The material was used in the next step without purification. LCMS (ES−): m/z 443.0 [M−H]⁻

### Step 2: 7-benzyloxy-5-fluoro-6-(1,1,4-trioxo-1,2,5-thiadiazolidin-2-yl)naphthalene-2-carboxylic acid (3)

Into a 100 mL single neck round bottom flask containing a well-stirred solution of methyl 7-benzyloxy-5-fluoro-6-(1,1,4-trioxo-1,2,5-thiadiazolidin-2-yl)naphthalene-2-carboxylate (2, 3.8 g, 5.30 mmol) in THF (5 mL) was added a solution of lithium hydroxide monohydrate (1.11 g, 26.51 mmol) in water (5 mL). After 16 h, the reaction mixture was acidified with aqueous 1.5 N HCl solution. The mixture was extracted with ethyl acetate (3×70 mL). The combined organic layer was washed with water (100 mL), brine (100 mL) and dried over sodium sulfate. The solvent was removed under reduced pressure and the residue purified by reverse phase column chromatography [Purification method: Biotage, C-18, 120 g column; Mobile phase A: 0.1% formic acid in water; Mobile phase B: Acetonitrile] to afford 7-benzyloxy-5-fluoro-6-(1,1,4-trioxo-1,2,5-thiadiazolidin-2-yl)naphthalene-2-carboxylic acid (3, 1.25 g, 2.89 mmol, 54% yield) as a colorless solid. LCMS (ES−): m/z 429.0 [M−H]⁻

### 5-[1-fluoro-3-hydroxy-7-[(3R)-pyrrolidin-3-yl]-2-naphthyl]-1,1-dioxo-1,2,5-thiadiazolidin-3-one (5a)

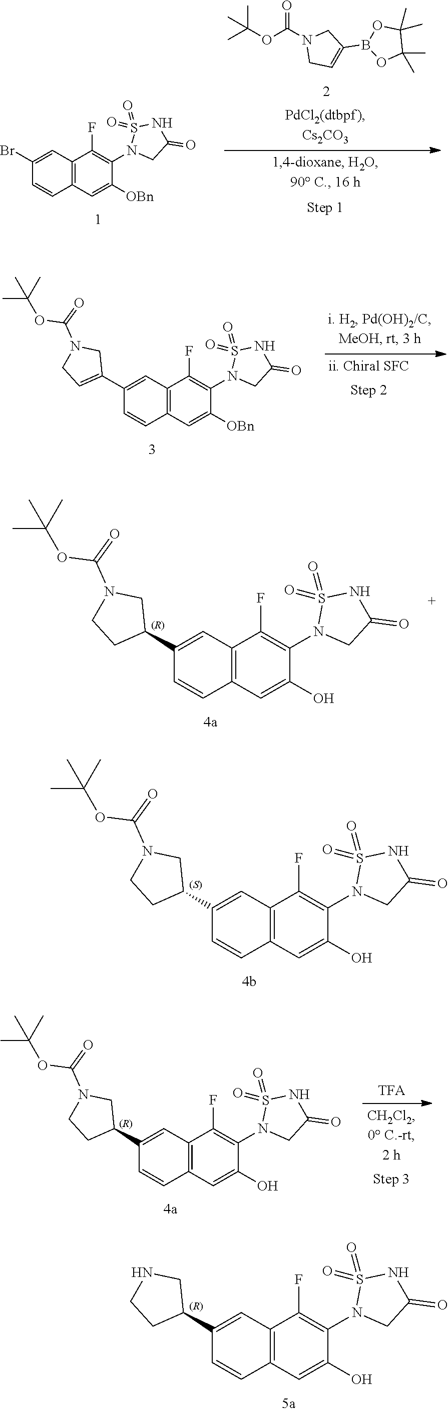

### Step 1: tert-butyl 3-[6-benzyloxy-8-fluoro-7-(1,1,4-trioxo-1,2,5-thiadiazolidin-2-yl)-2-naphthyl]-2,5-dihydropyrrole-1-carboxylate (3)

Into a 100 mL sealed-tube containing a well-stirred solution of 5-(3-benzyloxy-7-bromo-1-fluoro-2-naphthyl)-1,1-dioxo-1,2,5-thiadiazolidin-3-one (1, 1.3 g, 2.79 mmol) and tert-butyl 3-(4,4,5,5-tetramethyl-1,3,2-dioxaborolan-2-yl)-2,5-dihydropyrrole-1-carboxylate (2, 824.72 mg, 2.79 mmol) in dioxane (13 mL) and water (5.2 mL) was added Cs₂CO₃ (2.73 g, 8.38 mmol) under nitrogen atmosphere. The reaction mixture was degassed by bubbling nitrogen through the solution for 5 min. Subsequently, [1,1′-bis(di-tert-butylphosphino)ferrocene]dichloropalladium(II) (91.05 mg, 139.70 μmol) was added to the reaction mixture and the resulting suspension was heated at 90° C. for 16 h. The reaction mixture was cooled to ambient temperature, poured into water (100 mL) and extracted with EtOAc (2×150 mL). The organic layers were combined, dried (anhydrous Na₂SO₄), filtered and concentrated under reduced pressure and triturated with MTBE (20 mL) to afford tert-butyl 3-[6-benzyloxy-8-fluoro-7-(1,1,4-trioxo-1,2,5-thiadiazolidin-2-yl)-2-naphthyl]-2,5-dihydropyrrole-1-carboxylate (3, 1.12 g, 1.90 mmol, 68% yield) as a brown solid.

LCMS (ES−): m/z 552.1 [M−H]⁻

### Step 2: tert-butyl (3R)-3-[8-fluoro-6-hydroxy-7-(1,1,4-trioxo-1,2,5-thiadiazolidin-2-yl)-2-naphthyl]pyrrolidine-1-carboxylate (4a, First Eluted Enantiomer) and tert-butyl (3S)-3-[8-fluoro-6-hydroxy-7-(1,1,4-trioxo-1,2,5-thiadiazolidin-2-yl)-2-naphthyl]pyrrolidine-1-carboxylate (4b, Second Eluted Enantiomer)

Note: Configurations are arbitrarily assigned.

Into a 100 mL single-neck round-bottom flask containing a well-stirred solution of tert-butyl 3-[6-benzyloxy-8-fluoro-7-(1,1,4-trioxo-1,2,5-thiadiazolidin-2-yl)-2-naphthyl]-2,5-dihydropyrrole-1-carboxylate (3, 1.12 g, 1.88 mmol) in methanol (15 mL) was added Pd(OH)₂ on carbon (20 wt. % 50% water) (792.69 mg, 5.64 mmol) under nitrogen atmosphere at ambient temperature. The reaction was stirred under hydrogen bladder pressure for 3 h. The reaction was filtered through Celite and washed with methanol (150 mL). The filtrate was concentrated under reduced pressure and triturated with MTBE (25 mL) to afford the racemic product (4a/b, 830 mg, 98%, not shown) as a brown color solid.

The enantiomers were separated by chiral SFC [Method details: Column Name: Chiracel OZ-H; Flow rate: 5 mL/min; Co-Solvent: 40%; Co-Solvent Name: 0.5% Isopropyl amine in methanol; Outlet Pressure: 100 bar; Injected Volume: 15 μL; Temperature: 35° C.]

After concentration, the first eluted fraction at RT 3.97: tert-butyl (3R)-3-[8-fluoro-6-hydroxy-7-(1,1,4-trioxo-1,2,5-thiadiazolidin-2-yl)-2-naphthyl]pyrrolidine-1-carboxylate (4a, first eluted enantiomer, 280 mg, 581.00 μmol, 31% yield) was isolated as an off-white solid.

LCMS (ES−): m/z 464.0 [M−H]⁻

the second eluted fraction at RT 5.46: tert-butyl (3S)-3-[8-fluoro-6-hydroxy-7-(1,1,4-trioxo-1,2,5-thiadiazolidin-2-yl)-2-naphthyl]pyrrolidine-1-carboxylate (4b, second eluted enantiomer, 350 mg, 736.85 μmol, 39% yield) was isolated as an off-white solid.

LCMS (ES−): m/z 464.0 [M−H]⁻

### Step 3: 5-[1-fluoro-3-hydroxy-7-[(3R)-pyrrolidin-3-yl]-2-naphthyl]-1,1-dioxo-1,2,5-thiadiazolidin-3-one (5a)

Into a 50 mL single-neck round-bottom flask containing a well-stirred solution of tert-butyl (3R)-3-[8-fluoro-6-hydroxy-7-(1,1,4-trioxo-1,2,5-thiadiazolidin-2-yl)-2-naphthyl]pyrrolidine-1-carboxylate (4a, 280 mg, 577.45 μmol) in DCM (3 mL) was added TFA (1.32 g, 11.55 mmol, 889.74 μL) at 0° C. The reaction mixture was stirred at ambient temperature for 2 h. The reaction mixture was concentrated under vacuum and subjected to reverse-phase column chromatography [60 g of C18 column; Mobile Phase A: 0.1% TFA in water and Mobile Phase B: CH₃CN] to afford 5-[1-fluoro-3-hydroxy-7-[(3R)-pyrrolidin-3-yl]-2-naphthyl]-1,1-dioxo-1,2,5-thiadiazolidin-3-one (5a, 145 mg, 298.74 μmol, 52% yield, TFA salt) as an off-white solid.

LCMS (ES+): m/z 366.2 [M+H]⁺

### 5-[1-fluoro-3-hydroxy-7-[(3S)-pyrrolidin-3-yl]-2-naphthyl]-1,1-dioxo-1,2,5-thiadiazolidin-3-one (2)

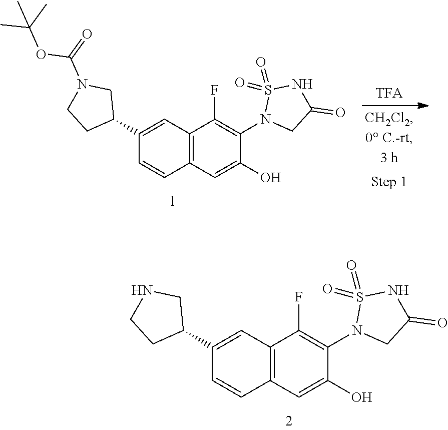 [SMILES: CC(C)(C)OC(=O)N1CC[C@@H](c2ccc3cc(O)c(N4CC(=O)NS4(=O)=O)c(F)c3c2)C1 ; O=C1CN(c2c(O)cc3ccc([C@@H]4CCNC4)cc3c2F)S(=O)(=O)N1]

### Step 1: 5-[1-fluoro-3-hydroxy-7-[(3S)-pyrrolidin-3-yl]-2-naphthyl]-1,1-dioxo-1,2,5-thiadiazolidin-3-one (2)

Into a 25 mL single neck round bottom flask containing a well-stirred solution of tert-butyl (3S)-3-[8-fluoro-6-hydroxy-7-(1,1,4-trioxo-1,2,5-thiadiazolidin-2-yl)-2-naphthyl]pyrrolidine-1-carboxylate (1, 200 mg, 423.98 μmol, second eluted isomer) in dry DCM (3 mL) was added TFA (740.00 mg, 6.49 mmol, 0.5 mL) under nitrogen atmosphere at 0° C. The reaction mixture was stirred for 3 h at ambient temperature. The reaction mixture was concentrated under reduced pressure and co-distilled with toluene (2×10 mL) and triturated with diethyl ether (10 mL) to afford 5-[1-fluoro-3-hydroxy-7-[(3S)-pyrrolidin-3-yl]-2-naphthyl]-1,1-dioxo-1,2,5-thiadiazolidin-3-one (2, 190 mg, 357.45 μmol, 84% yield, TFA salt) as a brown solid. LCMS (ES+): m/z 366.2 [M+H]⁺

### 5-[[6-benzyloxy-8-fluoro-7-(1,1,4-trioxo-1,2,5-thiadiazolidin-2-yl)-2-naphthyl]oxy]-3,3-dimethyl-pentanal (5)

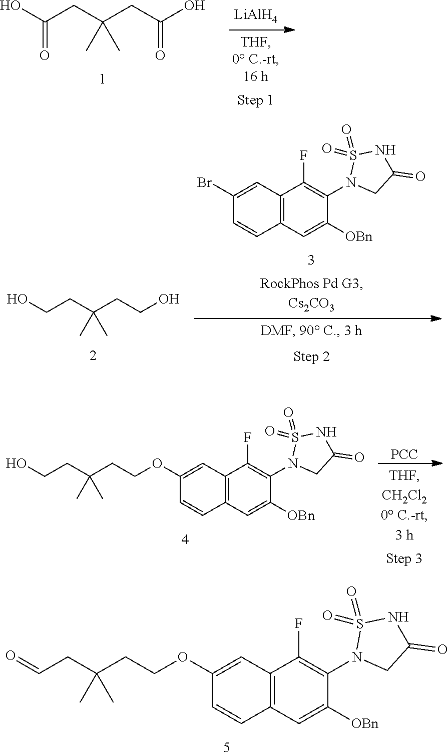

### Step 1: Preparation of 3,3-dimethylpentane-1,5-diol (2)

Into a 500 mL three-neck, round-bottom flask containing a well-stirred solution of 3,3-dimethylpentanedioic acid (1, 4 g, 24.97 mmol, 2.82 mL) in anhydrous THF (160 mL) was added LiALH₄ (2.0 M solution in THF, 99.88 mmol, 50 mL) at 0° C. The reaction mixture was stirred for 16 h at room temperature. The reaction mixture was quenched slowly with water (30 mL). 20 mL of saturated NaOH solution was added and stirred again for 10 mins. The reaction mixture was filtered through Celite and washed with EtOAc (400 mL). The filtrate was concentrated under reduced pressure to get crude 3,3-dimethylpentane-1,5-diol (2, 3.23 g, 23.70 mmol, 95% yield) as a yellow oil. The material was used in the next step without further purification.

### Step 2: 5-[3-benzyloxy-1-fluoro-7-(5-hydroxy-3,3-dimethyl-pentoxy)-2-naphthyl]-1,1-dioxo-1,2,5-thiadiazolidin-3-one (4)

Into a 25 mL sealed-tube containing a well-stirred solution of 5-(3-benzyloxy-7-bromo-1-fluoro-2-naphthyl)-1,1-dioxo-1,2,5-thiadiazolidin-3-one (3, 500 mg, 1.07 mmol) in DMF (5 mL) were added 3,3-dimethylpentane-1,5-diol (2, 1.46 g, 10.75 mmol) and Cs₂CO₃ (700.25 mg, 2.15 mmol). The reaction mixture was degassed by purging with nitrogen for 5 min. RockPhos Pd G3 (27.03 mg, 32.24 μmol) was added and the reaction mixture was heated at 90° C. for 3 h. The reaction mixture was filtered through Celite and washed with EtOAc (100 mL). Filtrate was concentrated under reduced pressure to get the crude material that was purified by reverse-phase column chromatography [Column: SiliaSep C18 120 g, Mobile phase: 0.1% TFA in Water/CH₃CN] to afford 5-[3-benzyloxy-1-fluoro-7-(5-hydroxy-3,3-dimethyl-pentoxy)-2-naphthyl]-1,1-dioxo-1,2,5-thiadiazolidin-3-one (4, 280 mg, 536.01 μmol, 50% yield) as an off-white solid.

LCMS (ES−): m/z 515.0 [M−H]⁻

### Step 3: 5-[[6-benzyloxy-8-fluoro-7-(1,1,4-trioxo-1,2,5-thiadiazolidin-2-yl)-2-naphthyl]oxy]-3,3-dimethyl-pentanal (5)

Into a 50 mL single-neck round-bottom flask containing a well-stirred solution of 5-[3-benzyloxy-1-fluoro-7-(5-hydroxy-3,3-dimethyl-pentoxy)-2-naphthyl]-1,1-dioxo-1,2,5-thiadiazolidin-3-one (4, 280 mg, 536.61 μmol) in THF (4.0 mL) and DCM (12 mL) was added PCC (347.01 mg, 1.61 mmol) at 0° C. The reaction mixture was stirred at room temperature for 3 h. The reaction mixture was filtered through Celite and washed with DCM (50 mL). The filtrate was concentrated under reduced pressure and trituration with MTBE (5 mL) afford 5-[[6-benzyloxy-8-fluoro-7-(1,1,4-trioxo-1,2,5-thiadiazolidin-2-yl)-2-naphthyl]oxy]-3,3-dimethyl-pentanal (5, 350 mg, 302.00 μmol, 56% yield) as a brown solid.

LCMS (ES−): m/z 513.0 [M−H]⁻

### 5-(3-(benzyloxy)-1-fluoro-7-(piperidin-4-yloxy)naphthalen-2-yl)-1,2,5-thiadiazolidin-3-one 1,1-dioxide (4)

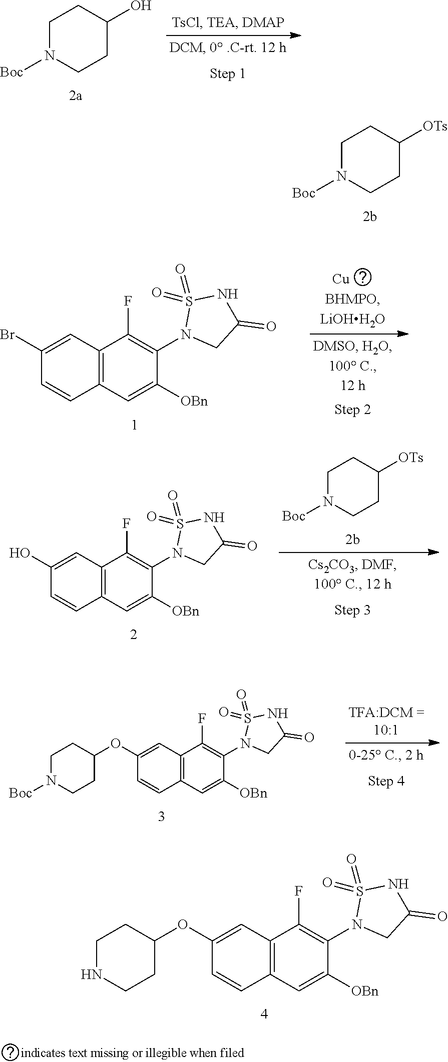

### Step 1: tert-butyl 4-(tosyloxy)piperidine-1-carboxylate (2b)

To a solution of tert-butyl 4-hydroxypiperidine-1-carboxylate (2a, 1 g, 4.97 mmol) in DCM (10 mL) was added triethylamine (1.01 g, 9.94 mmol, 1.39 mL) and N,N-dimethylpyridin-4-amine (20 mg, 163.71 μmol) followed by 4-methylbenzene-1-sulfonyl chloride (1.14 g, 5.96 mmol) at 0° C. The mixture was stirred at 25° C. for 12 h. The residue was diluted with DCM (20 mL) and water (30 mL). The layers were separated and the aqueous phase was extracted with DCM (2×20 mL). The organic layer was dried over Na₂SO₄, filtered, concentrated and purified by column chromatography on silica gel (petroleum ether/ethyl acetate=10/1 to 5/1) to afford tert-butyl 4-(tosyloxy)piperidine-1-carboxylate (2b, 1 g, 2.79 mmol, 56% yield) as a white solid.

LCMS (ESI): m/z 256.2 [M−Boc+H]⁺

### Step 2: 5-(3-(benzyloxy)-1-fluoro-7-hydroxynaphthalen-2-yl)-1,2,5-thiadiazolidin-3-one 1,1-dioxide (2)

To a solution of 5-(3-(benzyloxy)-7-bromo-1-fluoronaphthalen-2-yl)-1,2,5-thiadiazolidin-3-one 1,1-dioxide (1, 2 g, 4.30 mmol), bis[(Z)-1-methyl-3-oxo-but-1-enoxy]copper (100.00 mg, 382.03 μmol) and N₁,N₂-bis(4-hydroxy-2,6-dimethylphenyl)oxalamide (141.14 mg, 429.84 μmol) in DMSO (5 mL) was added a solution of LiOH H₂O (600.00 mg, 25.05 mmol) in water (1 mL) under N₂. The mixture was stirred at 100° C. for 12 h. The reaction was cooled to room temperature, filtered and purified by reversed phase column (C18, 120 g; condition: 0.1% formic acid water/acetonitrile) to afford 5-(3-(benzyloxy)-1-fluoro-7-hydroxynaphthalen-2-yl)-1,2,5-thiadiazolidin-3-one 1,1-dioxide (2, 1.23 g, 2.61 mmol, 61% yield, formic acid salt) as a brown solid.

LCMS (ESI): m/z 402.9 [M+H]⁺

### Step 3: tert-butyl 4-((6-(benzyloxy)-7-(1,1-dioxido-4-oxo-1,2,5-thiadiazolidin-2-yl)-8-fluoronaphthalen-2-yl)oxy)piperidine-1-carboxylate (3)

To a solution of tert-butyl 4-(tosyloxy)piperidine-1-carboxylate (2b, 540.00 mg, 1.52 mmol) and 5-(3-(benzyloxy)-1-fluoro-7-hydroxynaphthalen-2-yl)-1,2,5-thiadiazolidin-3-one 1,1-dioxide (2, 600 mg, 1.49 mmol) in DMF (15 mL) was added cesium carbonate (1.47 g, 4.51 mmol). The mixture was stirred at 100° C. for 12 h. After being cooled to room temperature, the mixture was diluted with EtOAc (20 mL) and water (20 mL). The layers were separated and the aqueous phase was extracted with EtOAc (2×20 mL). The organic layer was dried over Na₂SO₄, filtered and concentrated in vacuo. The residue was purified by reversed phase column (C18, 80 g; condition: 0.1% formic acid in water/acetonitrile) and concentrated in vacuo to give tert-butyl 4-((6-(benzyloxy)-7-(1,1-dioxido-4-oxo-1,2,5-thiadiazolidin-2-yl)-8-fluoronaphthalen-2-yl)oxy)piperidine-1-carboxylate (3, 410 mg, 629.60 μmol, 42% yield, formic acid salt) as a brown solid.

LCMS (ESI): m/z 486.2 [M−Boc+H]⁺

### Step 4: 5-(3-(benzyloxy)-1-fluoro-7-(piperidin-4-yloxy)naphthalen-2-yl)-1,2,5-thiadiazolidin-3-one 1,1-dioxide (4)

To a solution of tert-butyl 4-((6-(benzyloxy)-7-(1,1-dioxido-4-oxo-1,2,5-thiadiazolidin-2-yl)-8-fluoronaphthalen-2-yl)oxy)piperidine-1-carboxylate (3, 520 mg, 887.91 μmol) in DCM (10 mL) was added trifluoroacetic acid (1.48 g, 12.98 mmol, 1 mL) at 0° C. The mixture was stirred at 25° C. for 2 h. The mixture was concentrated in vacuum to give 5-(3-(benzyloxy)-1-fluoro-7-(piperidin-4-yloxy)naphthalen-2-yl)-1,2,5-thiadiazolidin-3-one 1,1-dioxide (4, 600 mg, crude, TFA salt) as a brown solid and it was used into next step without further purification.

LCMS (ESI): m/z 486.1 [M+H]⁺

### Phenyl (7-(benzyloxy)-6-(1,1-dioxido-4-oxo-1,2,5-thiadiazolidin-2-yl)-5-fluoronaphthalen-2-yl)carbamate (3)

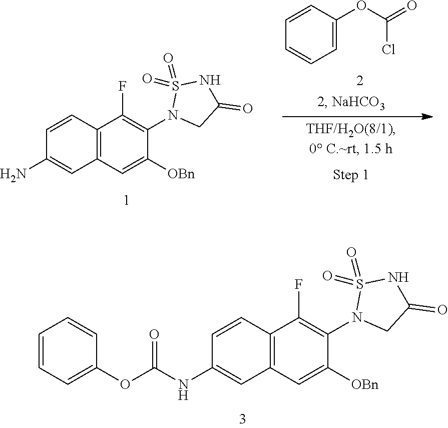 [SMILES: Nc1ccc2c(F)c(N3CC(=O)NS3(=O)=O)c(OCc3ccccc3)cc2c1 ; O=C(Cl)Oc1ccccc1 ; O=C1CN(c2c(OCc3ccccc3)cc3cc(NC(=O)Oc4ccccc4)ccc3c2F)S(=O)(=O)N1]

### Step 1: Phenyl (7-(benzyloxy)-6-(1,1-dioxido-4-oxo-1,2,5-thiadiazolidin-2-yl)-5-fluoronaphthalen-2-yl)carbamate (3)

To a mixture of 5-(6-amino-3-(benzyloxy)-1-fluoronaphthalen-2-yl)-1,2,5-thiadiazolidin-3-one 1,1-dioxide (1, 1 g, 1.94 mmol, TFA salt, 1 eq) in THF (80 mL) was added NaHCO₃ (609.76 mg, 7.26 mmol, 5.6 eq) in water (10 mL). Then to the mixture was added phenyl carbonochloridate (2, 304.36 mg, 1.94 mmol, 243.49 μL, 1.5 eq) at 0° C. and stirred at 20° C. for 1 h. To the reaction mixture was added phenyl carbonochloridate (2, 60.87 mg, 388.78 μmol, 48.70 μL, 0.3 eq) at 0° C. and stirred at 20° C. for 0.5 h. The reaction mixture was adjusted pH to 5 with 1 N HCl aqueous. Then the reaction mixture was diluted with water (70 mL) and extracted with ethyl acetate (70 mL×2). The combined organic layers were washed with brine (100 mL), dried over Na₂SO₄, filtered and concentrated under reduced pressure to give a residue. The residue was purified by flash silica gel chromatography (60 mL/min, Eluent of 0-100% ethyl acetate/petroleum ether and 0-20% dichloromethane/methanol, Column: ISCO; 10 g SepaFlash Silica Flash Column; DCM/MeOH=5/1) to afford phenyl (7-(benzyloxy)-6-(1,1-dioxido-4-oxo-1,2,5-thiadiazolidin-2-yl)-5-fluoronaphthalen-2-yl)carbamate (3, 880 mg, 1.65 mmol, 95% yield) as a red solid.

LCMS (ESI): m/z 522.1 [M+H]⁺

¹H NMR (400 MHz, d₆-DMSO) δ 10.65 (s, 1H), 8.11 (s, 1H), 7.98 (d, J=8.8 Hz, 1H), 7.60 (dd, J=1.6, 8.8 Hz, 1H), 7.54-7.50 (m, 2H), 7.49-7.44 (m, 2H), 7.41-7.33 (m, 4H), 7.32-7.27 (m, 3H), 5.29 (s, 2H), 4.55 (s, 2H).

### 5-(7-(Azetidine-3-yloxy)-3-(benzyloxy)-1-fluoronaphthalen-2-yl)-1,2,5-thiadiazolidin-3-one 1,1-dioxide (5)

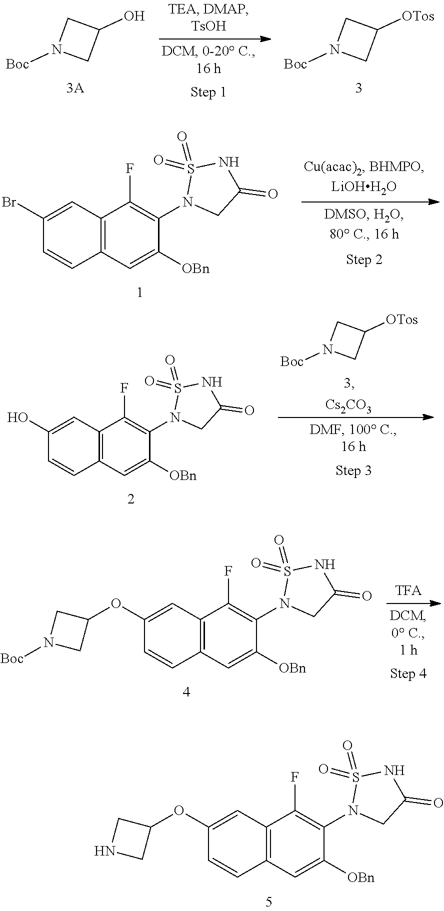

### Step 1: tert-butyl 3-(tosyloxy)azetidine-1-carboxylate (3)

To a solution of tert-butyl 3-hydroxyazetidine-1-carboxylate (3A, 5 g, 28.87 mmol), TEA (8.76 g, 86.60 mmol, 12.07 mL) in DCM (50 mL) was added DMAP (352.66 mg, 2.89 mmol) and TsOH (9.94 g, 57.73 mmol) at 0° C. The mixture was stirred at 20° C. for 16 h under N₂. The reaction mixture was concentrated under reduced pressure. The residue purified by column chromatography (SiO₂, Petroleum ether:Ethyl acetate=1:0 to 1:1 to afford tert-butyl 3-(tosyloxy)azetidine-1-carboxylate (3, 5 g, 15.27 mmol, 53% yield) as a yellow oil.

LCMS (ESI): m/z 272.2 [M−^{t}Bu+H]⁺

### Step 2: 5-(3-(benzyloxy)-1-fluoro-7-hydroxynaphthalen-2-yl)-1,2,5-thiadiazolidin-3-one 1,1-dioxide (2)

To a mixture of 5-(3-(benzyloxy)-7-bromo-1-fluoronaphthalen-2-yl)-1,2,5-thiadiazolidin-3-one 1,1-dioxide (1 g, 2.15 mmol), LiOH·H₂O (189.39 mg, 4.51 mmol, 125.43 μL) and BHMPO (70.56 mg, 214.92 μmol) in DMSO (8 mL), Water (2 mL) was added Cu(acac)₂ (56.26 mg, 214.92 μmol) under N₂. The mixture was stirred at 80° C. for 16 h under N₂. The residue was diluted with ice H₂O (100 mL) and extracted with ethyl acetate (100 mL×2). The combined organic layers were washed with brine (100 mL), dried over Na₂SO₄, filtered and concentrated under reduced pressure to afford 5-(3-(benzyloxy)-1-fluoro-7-hydroxynaphthalen-2-yl)-1,2,5-thiadiazolidin-3-one 1,1-dioxide (2, 680 mg, 1.69 mmol, 79% yield), which was used to the next step without further purification.

LCMS (ESI): m/z 403.1 [M+H]⁺

### Step 3: tert-butyl 3-((6-(benzyloxy)-7-(1,1-dioxido-4-oxo-1,2,5-thiadiazolidin-2-yl)-8-fluoronaphthalen-2-yl)oxy)azetidine-1-carboxylate (4)

To a solution of 5-(3-(benzyloxy)-1-fluoro-7-hydroxynaphthalen-2-yl)-1,2,5-thiadiazolidin-3-one 1,1-dioxide (2, 680 mg, 1.69 mmol, 1 eq), tert-butyl 3-(tosyloxy)azetidine-1-carboxylate (3, 1.11 g, 3.38 mmol, 2 eq) in DMF (10 mL) was added Cs₂CO₃ (1.38 g, 4.22 mmol, 2.5 eq). The mixture was stirred at 100° C. for 16 h under N₂. The reaction mixture was concentrated under reduced pressure. The reaction solution was purified by prep-HPLC (flow: 25 mL/min; gradient: from 64-34% water (0.1% TFA)-ACN; column: Phenomenex Luna C18 150×25 mm×10 um) to afford tert-butyl 3-((6-(benzyloxy)-7-(1,1-dioxido-4-oxo-1,2,5-thiadiazolidin-2-yl)-8-fluoronaphthalen-2-yl)oxy)azetidine-1-carboxylate (4, 450 mg, 807.05 μmol, 48% yield) as a white solid.

LCMS (ESI): m/z 502.1 [M−^{t}Bu+H]⁺

### Step 4: 5-(7-(azetidin-3-yloxy)-3-(benzyloxy)-1-fluoronaphthalen-2-yl)-1,2,5-thiadiazolidin-3-one 1,1-dioxide (5)

To a solution of tert-butyl 3-((6-(benzyloxy)-7-(1,1-dioxido-4-oxo-1,2,5-thiadiazolidin-2-yl)-8-fluoronaphthalen-2-yl)oxy)azetidine-1-carboxylate (4, 400 mg, 717.37 μmol) in DCM (8 mL) was added TFA (11.84 g, 103.84 mmol, 8 mL) at 0° C. The mixture was stirred at 0° C. for 1 h. The reaction mixture was concentrated under reduced pressure to afford 5-(7-(azetidin-3-yloxy)-3-(benzyloxy)-1-fluoronaphthalen-2-yl)-1,2,5-thiadiazolidin-3-one 1,1-dioxide (5, 300 mg, 524.94 μmol, 73% yield), which was used in the next step without further purification.

LCMS (ESI): m/z 458.3 [M+H]⁺

### (E)-3-[7-benzyloxy-5-fluoro-6-(1,1,4-trioxo-1,2,5-thiadiazolidin-2-yl)-2-naphthyl]prop-2-enal (4)

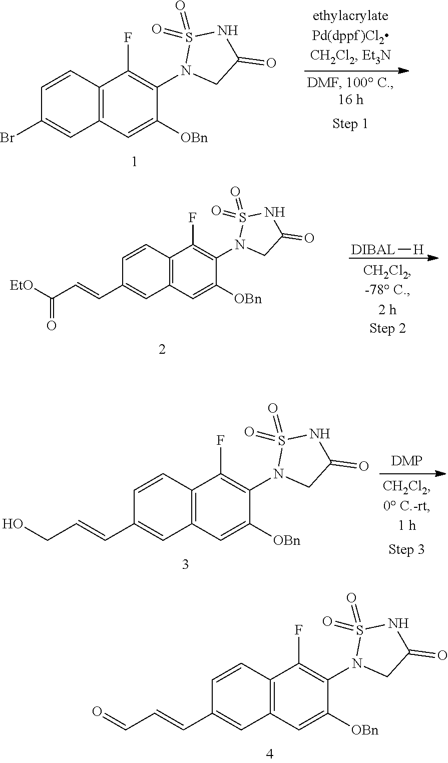

### Step 1: ethyl (E)-3-[7-benzyloxy-5-fluoro-6-(1,1,4-trioxo-1,2,5-thiadiazolidin-2-yl)-2-naphthyl]prop-2-enoate (2)

Into a 100 mL sealed-tube containing a well-stirred solution of 5-(3-benzyloxy-6-bromo-1-fluoro-2-naphthyl)-1,1-dioxo-1,2,5-thiadiazolidin-3-one (1, 500 mg, 1.07 mmol) in DMF (10 mL) was added ethyl acrylate (430.33 mg, 4.30 mmol, 465.73 μL) and triethylamine (543.69 mg, 5.37 mmol, 748.88 μL) at room temperature and the resulting reaction mixture was degassed for 5 min. Then [1,1′-bis(diphenylphosphino)ferrocene]dichloropalladium(II), complex with dichloromethane (87.76 mg, 107.46 μmol) was added and the reaction mixture was stirred at 110° C. for 16 h. The mixture was filtered through Celite and washed with DCM. The filtrate was evaporated under vacuum to get the crude material that was purified by silicagel flash column chromatography (230-400 mesh, 5% DCM in CH₃OH) to obtain ethyl (E)-3-[7-benzyloxy-5-fluoro-6-(1,1,4-trioxo-1,2,5-thiadiazolidin-2-yl)-2-naphthyl]prop-2-enoate (2, 550 mg, 1.03 mmol, 96% yield) as a brown solid.

LCMS (ES−): m/z 483.0 [M−H]⁻

### Step 2: 5-[3-benzyloxy-1-fluoro-6-[(E)-3-hydroxyprop-1-enyl]-2-naphthyl]-1,1-dioxo-1,2,5-thiadiazolidin-3-one (3)

Into a 100 mL two-neck round bottom flask containing a well-stirred solution of ethyl (E)-3-[7-benzyloxy-5-fluoro-6-(1,1,4-trioxo-1,2,5-thiadiazolidin-2-yl)-2-naphthyl]prop-2-enoate (2, 550 mg, 1.03 mmol) in CH₂Cl₂ (10 mL) at −78° C., was added DIBAL-H (583.80 mg, 2.05 mmol, 2.05 mL) and the reaction mixture was stirred at −78° C. for 2 h. The mixture was quenched with a saturated solution of sodium potassium tartrate and slowly warmed to room temperature. The mixture was extracted with CH₂Cl₂ (2×100 mL) and then washed with brine solution (10 mL). The combined organic layers were dried over Na₂SO₄, solvent removed and purified by silicagel flash column chromatography (230-400 mesh, 5% DCM in CH₃OH) to obtain 5-[3-benzyloxy-1-fluoro-6-[(E)-3-hydroxyprop-1-enyl]-2-naphthyl]-1,1-dioxo-1,2,5-thiadiazolidin-3-one (3, 500 mg, 980.66 μmol, 95% yield) as a yellow solid.

LCMS (ES−): m/z 441.0 [M−H]⁻

### Step 3: (E)-3-[7-benzyloxy-5-fluoro-6-(1,1,4-trioxo-1,2,5-thiadiazolidin-2-yl)-2-naphthyl]prop-2-enal (4)

Into a 50 mL single-neck, round-bottom flask containing a well-stirred solution of 5-[3-benzyloxy-1-fluoro-6-[(E)-3-hydroxyprop-1-enyl]-2-naphthyl]-1,1-dioxo-1,2,5-thiadiazolidin-3-one (3, 500 mg, 971.84 μmol) in CH₂Cl₂ (5 mL) was added Dess-Martin periodinane (824.39 mg, 1.94 mmol) at 0° C. After 1 h, the reaction mixture was quenched with a 1:1 mixture of sodium bicarbonate and sodium thiosulfate solution (20 mL). The solution was extracted with CH₂Cl₂ (2×10 mL). The combined organic layers were washed with water (20 mL), brine (20 mL), dried over Na₂SO₄ and then evaporated to give (E)-3-[7-benzyloxy-5-fluoro-6-(1,1,4-trioxo-1,2,5-thiadiazolidin-2-yl)-2-naphthyl]prop-2-enal (4, 550 mg, 363.38 μmol, 37% yield) as a brown solid.

LCMS (ES−): m/z 439.0 [M−H]⁻

### N-(7-(benzyloxy)-6-(1,1-dioxido-4-oxo-1,2,5-thiadiazolidin-2-yl)-5-fluoronaphthalen-2-yl)-2-bromoacetamide (5)

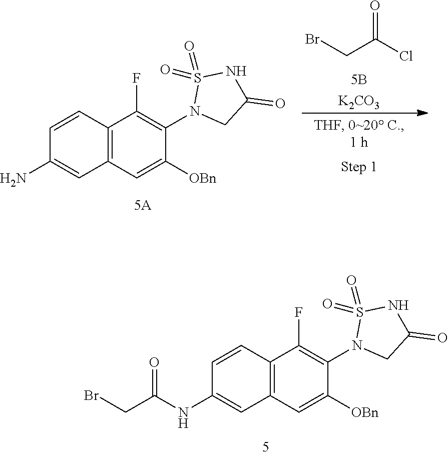

### Step 1: N-(7-(benzyloxy)-6-(1,1-dioxido-4-oxo-1,2,5-thiadiazolidin-2-yl)-5-fluoronaphthalen-2-yl)-2-bromoacetamide (5)

To a solution of 5-(6-amino-3-(benzyloxy)-1-fluoronaphthalen-2-yl)-1,2,5-thiadiazolidin-3-one 1,1-dioxide (5A, 150 mg, 291.02 μmol, formic acid salt) in THF (3.0 mL) was added K₂CO₃ (100.55 mg, 727.54 μmol). Then 2-bromoacetyl chloride (5B, 50.38 mg, 320.12 μmol) was added at 0° C. The mixture was stirred at 20° C. for 1 h. The reaction mixture was diluted with DMF (1 mL), filtered and concentrated under reduced pressure at 45° C. to afford N-(7-(benzyloxy)-6-(1,1-dioxido-4-oxo-1,2,5-thiadiazolidin-2-yl)-5-fluoronaphthalen-2-yl)-2-bromoacetamide (5, 155 mg, 216.62 μmol, 74% yield) as yellow oil.

LCMS (ESI): m/z 523.9 [M+H]⁺

### ((S)-5-(3-(benzyloxy)-1-fluoro-7-(pyrrolidin-3-yloxy)naphthalen-2-yl)-1,2,5-thiadiazolidin-3-one 1,1-dioxide

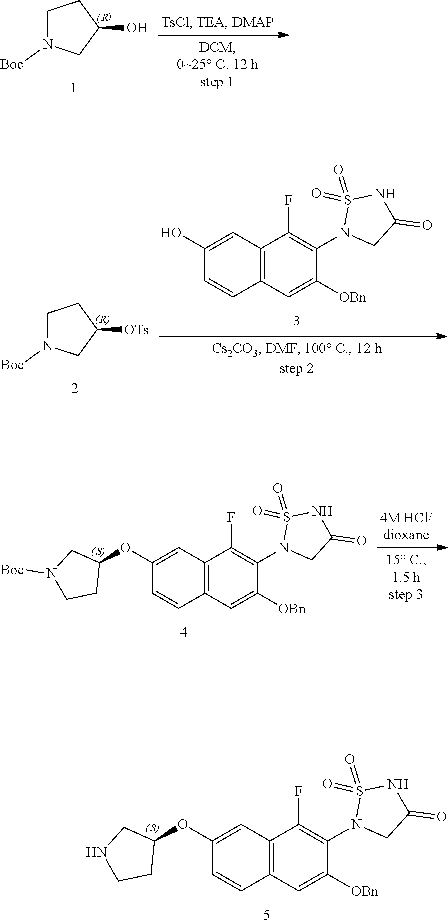

### Step 1: (R)-tert-butyl 3-(tosyloxy)pyrrolidine-1-carboxylate (2)

To a solution of tert-butyl (3R)-3-hydroxypyrrolidine-1-carboxylate (1, 3 g, 16.02 mmol), N,N-diethylethanamine (3.24 g, 32.05 mmol, 4.47 mL), N,N-dimethylpyridin-4-amine (293.62 mg, 2.40 mmol) were dissolved in DCM (50 mL), 4-methylbenzenesulfonyl chloride (4.58 g, 24.03 mmol) was added at 0° C., the reaction mixture was stirred at 20° C. for 16 h. The reaction mixture was purified by silica gel column chromatography (petroleum ether/ethyl acetate=2/1) to obtain the target (R)-tert-butyl 3-(tosyloxy)pyrrolidine-1-carboxylate (2, 2.5 g, 6.96 mmol, 43% yield) as a colorless oil.

LCMS (ESI): m/z 286.1 [M+H−t-Bu]⁺

### Step 2: tert-butyl (3S)-3-[[6-benzyloxy-8-fluoro-7-(1,1,4-trioxo-1,2,5-thiadiazolidin-2-yl)-2-naphthyl]oxy]pyrrolidine-1-carboxylate (4)

To a solution of (R)-tert-butyl 3-(tosyloxy)pyrrolidine-1-carboxylate (2, 286.95 mg, 840.47 μmol) 5-(3-(benzyloxy)-1-fluoro-7-hydroxynaphthalen-2-yl)-1,2,5-thiadiazolidin-3-one 1,1-dioxide (3, 380 mg, 840.47 μmol) in DMF (5 mL) was added cesium carbonate (821.52 mg, 2.52 mmol). The mixture was stirred at 100° C. for 12 h. The reaction was purified by reversed phase column chromatography (0.1% FA) to obtain tert-butyl (3S)-3-((6-(benzyloxy)-7-(1,1-dioxido-4-oxo-1,2,5-thiadiazolidin-2-yl)-8-fluoronaphthalen-2-yl)oxy)pyrrolidine-1-carboxylate (4, 280 mg, 465.35 μmol, 55% yield) LCMS (ESI): m/z 516.2 [M+H−t-Bu]⁺

### Step 3: ((S)-5-(3-(benzyloxy)-1-fluoro-7-(pyrrolidin-3-yloxy)naphthalen-2-yl)-1,2,5-thiadiazolidin-3-one 1,1-dioxide (5)

To a solution of tert-butyl (3S)-3-((6-(benzyloxy)-7-(1,1-dioxido-4-oxo-1,2,5-thiadiazolidin-2-yl)-8-fluoronaphthalen-2-yl)oxy)pyrrolidine-1-carboxylate (4, 50 mg, 87.47 μmol) was added HCl/dioxane (4 M, 2 ml). The mixture was stirred at 25° C. for 1 h. The reaction was concentrated to afford ((S)-5-(3-(benzyloxy)-1-fluoro-7-(pyrrolidin-3-yloxy)naphthalen-2-yl)-1,2,5-thiadiazolidin-3-one 1,1-dioxide (5, 44 mg, 77.96 μmol, 89% yield), which was used for the next step without further purification.

LCMS (ESI): m/z 472.1 [M+H]+

### (R)-5-(3-(benzyloxy)-1-fluoro-7-(pyrrolidin-3-yloxy)naphthalen-2-yl)-1,2,5-thiadiazolidin-3-one 1,1-dioxide (14)

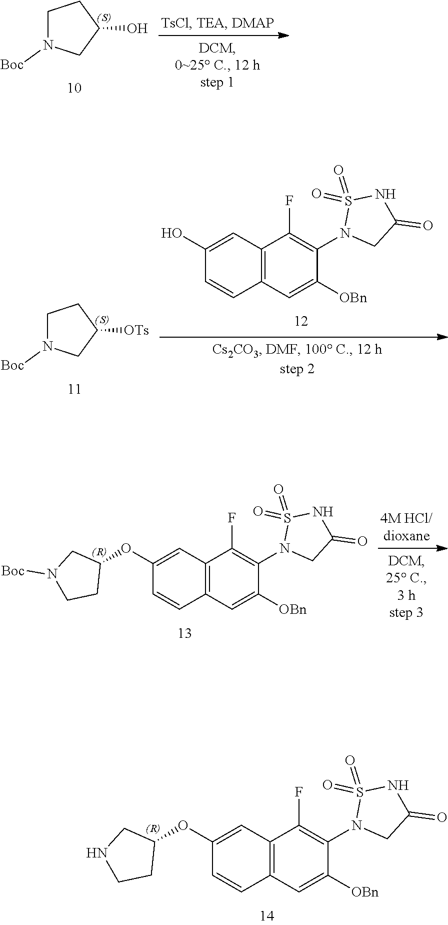

### Step 1: (S)-tert-butyl 3-(tosyloxy)pyrrolidine-1-carboxylate (11)

To a solution of (S)-tert-butyl 3-hydroxypyrrolidine-1-carboxylate (10, 3 g, 16.02 mmol), DMAP (195.74 mg, 1.60 mmol), TEA (4.86 g, 48.07 mmol, 6.70 mL) in DCM (35 mL) was added TsCl (6.11 g, 32.05 mmol) at 0° C., the reaction mixture was stirred at 30° C. for 16 h. The reaction mixture was concentrated under vacuum. The residue was purified by flash silica gel chromatography (flow: 60 mL/min; gradient: 0-100% ethylacetate in petroleum ether; ISCO®; 80 g SepaFlash®Silica Flash Column) to afford (S)-tert-butyl 3-(tosyloxy)pyrrolidine-1-carboxylate (11, 5 g, 14.64 mmol, 91% yield) as colorless oil.

LCMS (ESI): m/z 286.0 [M-tBu+H]⁺

SFC: ee % 100%

¹HNMR (400 MHz, DMSO-d6) δ 7.82 (d, J=8.4 Hz, 2H), 7.49 (d, J=8.0 Hz, 2H), 5.05 (br s, 1H), 3.39-3.18 (m, 4H), 2.43 (s, 3H), 2.12-1.86 (m, 2H), 1.37 (br d, J=11.6 Hz, 9H).

### Step 2: (R)-tert-butyl 3-((6-(benzyloxy)-7-(1,1-dioxido-4-oxo-1,2,5-thiadiazolidin-2-yl)-8-fluoronaphthalen-2-yl)oxy)pyrrolidine-1-carboxylate (13)

To a solution of tert-butyl (3S)-3-(p-tolylsulfonyloxy)pyrrolidine-1-carboxylate (11, 400 mg, 1.17 mmol), 5-(3-(benzyloxy)-1-fluoro-7-hydroxynaphthalen-2-yl)-1,2,5-thiadiazolidin-3-one 1,1-dioxide (12, 430 mg, 1.07 mmol) in DMF (5 mL) was added Cs₂CO₃ (1.15 g, 3.51 mmol). The mixture was stirred at 100° C. for 12 h. The reaction was quenched with water 10 (mL), extracted with EtOAc (20 mL*3) and concentrated to give (R)-tert-butyl 3-((6-(benzyloxy)-7-(1,1-dioxido-4-oxo-1,2,5-thiadiazolidin-2-yl)-8-fluoronaphthalen-2-yl)oxy)pyrrolidine-1-carboxylate (13, 700 mg, 1.14 mmol, 97% yield) as yellow oil.

LCMS (ESI): m/z 516.0 [M-tBu+H]⁺

SFC: ee % 100%

### Step 3: (R)-5-(3-(benzyloxy)-1-fluoro-7-(pyrrolidin-3-yloxy)naphthalen-2-yl)-1,2,5-thiadiazolidin-3-one 1,1-dioxide (14)

To a solution of tert-butyl (3R)-3-[[6-benzyloxy-8-fluoro-7-(1,1,4-trioxo-1,2,5-thiadiazolidin-2-yl)-2-naphthyl]oxy]pyrrolidine-1-carboxylate (13, 250 mg, 437.36 μmol) in DCM (5 mL) was added HCl/dioxane (4 M, 1.2 mL). The reaction mixture was stirred at 25° C. for 3 h. The mixture was concentrated to afford (R)-5-(3-(benzyloxy)-1-fluoro-7-(pyrrolidin-3-yloxy)naphthalen-2-yl)-1,2,5-thiadiazolidin-3-one 1,1-dioxide (14, 200 mg, 381.92 μmol, 87% yield, HCl salt) as yellow oil.

LCMS (ESI): m/z 472.0 [M+H]⁺

### 1-(2,6-dibenzyloxy-3-pyridyl)-3-methyl-5-(4,4,5,5-tetramethyl-1,3,2-dioxaborolan-2-yl)benzimidazol-2-one (3)

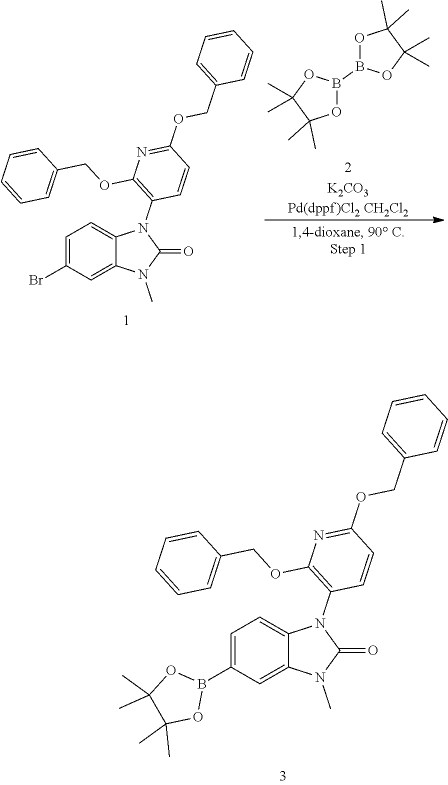

### Step 1:1-(2,6-dibenzyloxy-3-pyridyl)-3-methyl-5-(4,4,5,5-tetramethyl-1,3,2-dioxaborolan-2-yl)benzimidazol-2-one (3)

Into a 50 mL sealed tube containing a well-stirred solution of 5-bromo-1-(2,6-dibenzyloxy-3-pyridyl)-3-methyl-benzimidazol-2-one (1, 1 g, 1.87 mmol), Bis(pinacolato)diboron (2, 1.42 g, 5.60 mmol) in anhydrous 1,4-Dioxane (10 mL) was added potassium acetate (549.70 mg, 5.60 mmol, 350.13 μL). Nitrogen gas was purged through a reaction mixture for 15 min. Then Pd(dppf)C12-DCM (152.47 mg, 186.70 μmol) was added and stirring continued for 5 h at 90° C. The mixture was filtered through Celite, concentrated under reduced pressure, and purified by flash silica gel (230-400 mesh) column chromatography (40% EtOAc in Pet-ether) to afford 1-(2,6-dibenzyloxy-3-pyridyl)-3-methyl-5-(4,4,5,5-tetramethyl-1,3,2-dioxaborolan-2-yl)benzimidazol-2-one (3, 0.8 g, 1.32 mmol, 71% yield) as a white solid. LCMS (ES⁺): m/z 564.5 [M+H]⁺.

### 3-(3-methyl-2-oxo-5-(piperidin-4-yl)-2,3-dihydro-1H-benzo[d]imidazol-1-yl)piperidine-2,6-dione (10)

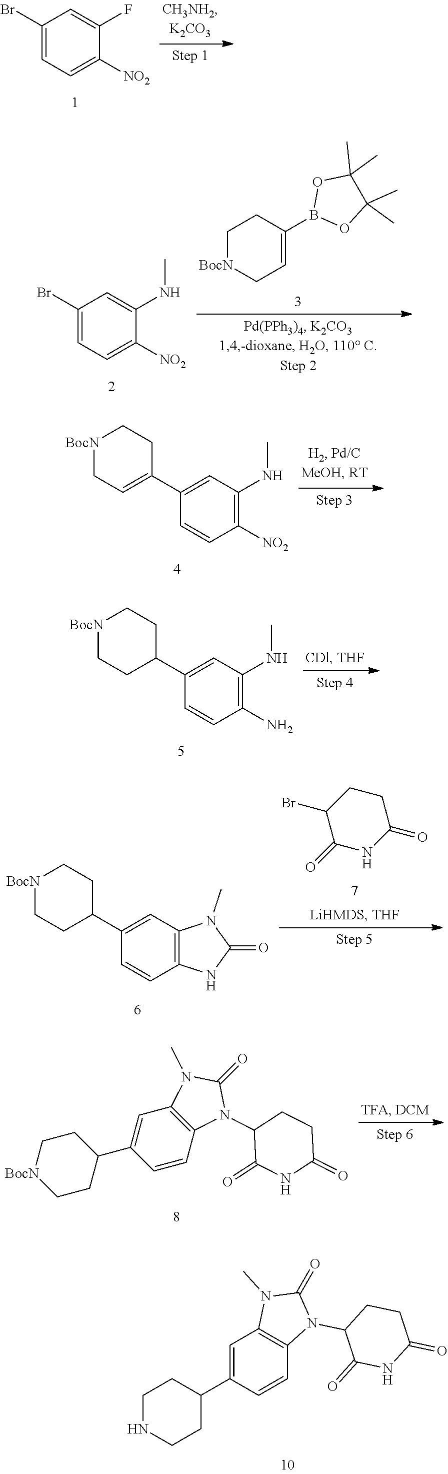

### Step 1: 5-bromo-N-methyl-2-nitroaniline (2)

To a stirred solution of 4-bromo-2-fluoro-1-nitrobenzene (1, 300 g, 1.36 mol) in DCM (3 L) were added K₂CO₃ (0.94 Kg, 6.8 mol) and methylamine (2M in THF) (2.04 L, 4.09 mol) and stirred for 16 h. Two batches of the reaction were combined. The reaction mixture was diluted with water (3.0 L) and extracted with DCM (2.5 L×2). The combined organic layer was washed with saturated sodium bicarbonate solution (1.5 L×2) and brine (1.5 L×2). The organic layer was dried over sodium sulfate, filtered and solvent removed under reduced pressure to obtain 5-bromo-N-methyl-2-nitroaniline (2, 600 g, 95% yield) as a yellow solid. LCMS (ES+): m/z 231.1 [M+H]⁺

### Step 2: tert-butyl 4-(3-(methylamino)-4-nitrophenyl)-3,6-dihydropyridine-1 (2H)-carboxylate (4)

To a stirred solution of 5-bromo-N-methyl-2-nitroaniline (2, 75.0 g, 0.326 mol) in 1,4-dioxane (1.2 L) and water (0.3 L) was added K₂CO₃ (270.3 g, 1.956 mol) and the mixture was stirred for 5 min. tert-Butyl 4-(4,4,5,5-tetramethyl-1,3,2-dioxaborolan-2-yl)-3,6-dihydropyridine-1 (2H)-carboxylate (3, 151.0 g, 0.489 mol) was added to the reaction mixture under nitrogen atmosphere and the reaction mixture was purged with nitrogen for 10 min. Palladium(0) tetrakis(triphenylphosphine) (37.66 g, 0.032 mol) was added to the reaction under nitrogen atmosphere. After purging with nitrogen for 10 min, the reaction was stirred at 110° C. for 4 h. Two batches of the reaction were combined. The reaction mixture was cooled to rt and filtered through Celite. The filtrate was diluted with water (1.5 L) and extracted with ethyl acetate (500 mL×2). The combined organic layer was washed with brine, dried over sodium sulfate, filtered and solvent removed under reduced pressure. The residue was purified by silica gel chromatography (0-20% EtOAc in petroleum ether as an eluent) to obtain tert-butyl 4-(3-(methylamino)-4-nitrophenyl)-3,6-dihydropyridine-1 (2H)-carboxylate (4, 150 g, 69% yield) as a red solid. LCMS (ES⁺): m/z 334.3 [M+H]⁺

### Step 3: tert-butyl 4-(4-amino-3-(methylamino)phenyl)piperidine-1-carboxylate (5)

A solution of tert-butyl 4-(3-(methylamino)-4-nitrophenyl)-3,6-dihydropyridine-1 (2H)-carboxylate (4, 50 g, 0.149 mol) in methanol (1 L) in a Parr-shaker flask was degassed. Palladium on carbon (10%, wet) (25.0 g) was added and the reaction mixture was put under an atmosphere of hydrogen (70-75 psi). Four batches were combined. After 8 h, the reaction mixture was filtered through Celite, washing with methanol. The filtrate was evaporated under reduced pressure and the residue purified by silica gel chromatography (0-20% ethyl acetate and petroleum ether) to obtain tert-butyl 4-(4-amino-3-(methylamino)phenyl)piperidine-1-carboxylate (5, 120.0 g, 65% yield) as dark brown solid. LCMS (ES−): m/z 304.2 [M−H]⁻

### Step 4: tert-butyl 4-(3-methyl-2-oxo-2,3-dihydro-1H-benzo[d]imidazol-5-yl)piperidine-1-carboxylate (6)

To a stirred solution of tert-butyl 4-(4-amino-3-(methylamino)phenyl)piperidine-1-carboxylate (5, 60 g, 0.196 mol) in THF (900 mL) at 0° C. was added CDI (33.45 g, 0.206 mol) and the reaction mixture was stirred at room temperature overnight. Two batches were combined. The solvent was removed under reduced pressure. The residue was triturated with MTBE and filtered to afford tert-butyl 4-(3-methyl-2-oxo-2,3-dihydro-1H-benzo[d]imidazol-5-yl)piperidine-1-carboxylate (6, 88.0 g, 68% yield) as an off white solid. LCMS (ES⁺): m/z 332.3 [M+H]⁺

### Step 5: tert-butyl 4-(1-(2,6-dioxopiperidin-3-yl)-3-methyl-2-oxo-2,3-dihydro-1H-benzo[d]imidazol-5-yl)piperidine-1-carboxylate (8)

To an ice cold stirred solution of tert-butyl 4-(3-methyl-2-oxo-2,3-dihydro-1H-benzo[d]imidazol-5-yl)piperidine-1-carboxylate (6, 44 g, 0.133 mol) in anhydrous THF (900 mL) was added 1 M LiHMDS (403 ml, 0.387 mol). The reaction mixture was stirred for 10 min before adding 3-bromopiperidine-2,6-dione (7, 43.34 g, 0.225 mol). The reaction mixture was stirred at 70-75° C. for 16 h. Two batches were combined. The reaction mixture was cooled to 0° C. and quenched by slow addition of aqueous 1N HCl (620 mL). The mixture was diluted with EtOAc (1 L), and the layers separated. The organic layer was washed with 0.5 N HCl (1.4 L), water (1.5 L×2) and brine (1.5 L). The combined organic layer was dried over sodium sulfate, filtered and concentrated under reduced pressure. The residue was purified by silica gel chromatography (20-50% EtOAc in Petroleum ether) to obtain tert-butyl 4-(1-(2,6-dioxopiperidin-3-yl)-3-methyl-2-oxo-2,3-dihydro-1H-benzo[d]imidazol-5-yl)piperidine-1-carboxylate (8, 51.0 g, 43% yield) as a grey off-white solid. LCMS (ES⁻): m/z 441.1[M−H]⁻

### Step 6: 3-(3-methyl-2-oxo-5-(piperidin-4-yl)-2,3-dihydro-1H-benzo[d]imidazol-1-yl)piperidine-2,6-dione (10)

To a stirred solution of tert-butyl 4-(1-(2,6-dioxopiperidin-3-yl)-3-methyl-2-oxo-2,3-dihydro-1H-benzo[d]imidazol-5-yl)piperidine-1-carboxylate (8, 25.5 g, 0.057 moles) in DCM (250 mL) at 0° C. was added TFA (87.2 ml) via dropwise addition. The reaction mixture was stirred at room temperature for 4 h. Two batches were combined. The volatiles were evaporated under reduced pressure and azeotroped twice with toluene. The residue was triturated with diethyl ether and dried under reduced pressure to obtain 3-(3-methyl-2-oxo-5-(piperidin-4-yl)-2,3-dihydro-1H-benzo[d]imidazol-1-yl)piperidine-2,6-dione (10, 26 g, 43.12 mmol, TFA salt) as an off white solid. LCMS (ES+): m/z 343.3[M+H]⁺

### 2-(1-(1-(2,6-dioxopiperidin-3-yl)-3-methyl-2-oxo-2,3-dihydro-1H-benzo[d]imidazol-5-yl)piperidin-4-yl)acetic acid (5)

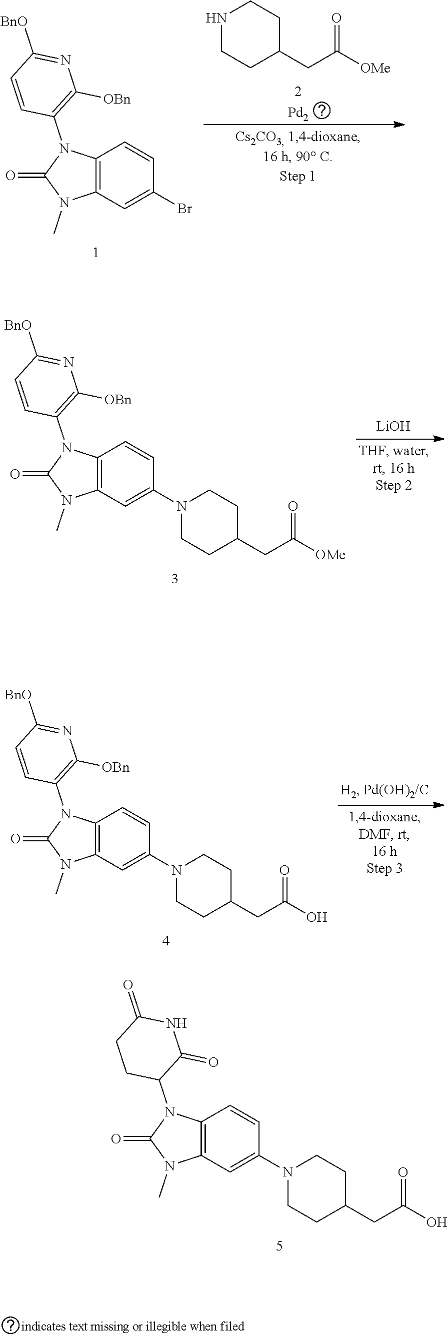

### Step 1: Methyl 2-(1-(1-(2,6-bis(benzyloxy)pyridin-3-yl)-3-methyl-2-oxo-2,3-dihydro-1H-benzo[d]imidazol-5-yl)piperidin-4-yl)acetate (3)

Into a 50 mL sealed tube containing a well-stirred solution of 5-bromo-1-(2,6-dibenzyloxy-3-pyridyl)-3-methyl-benzimidazol-2-one (1, 2.0 g, 3.87 mmol) and methyl 2-(4-piperidyl)acetate (2, 791.55 mg, 5.04 mmol) in 1,4-dioxane (20 mL) was added Cs₂CO₃ (3.79 g, 11.62 mmol). The reaction mixture was degassed with nitrogen for 10 min, then Pd₂(dba)₃ (532.00 mg, 580.96 μmol) and XPhos (461.58 mg, 968.26 μmol) were added. The reaction mixture was heated to 90° C. for 16 h. The reaction mixture was cooled to rt, filtered through Celite and washed with EtOAc (50 mL). The solvent was removed under reduced pressure and the residue purified by silica gel column chromatography (60-120 mesh, 50 g; 40-60% EtOAc in petroleum ether) to afford Methyl 2-(1-(1-(2,6-bis(benzyloxy)pyridin-3-yl)-3-methyl-2-oxo-2,3-dihydro-1H-benzo[d]imidazol-5-yl)piperidin-4-yl)acetate (3, 1.2 g, 52% yield) as a brown gummy solid.

LCMS (ES+): m/z 593.2 [M+H]⁺.

### Step 2: 2-(1-(1-(2,6-bis(benzyloxy)pyridin-3-yl)-3-methyl-2-oxo-2,3-dihydro-1H-benzo[d]imidazol-5-yl)piperidin-4-yl)acetic acid (4)

Into a 50 mL single neck, round bottom flask containing a well-stirred solution of Methyl 2-(1-(1-(2,6-bis(benzyloxy)pyridin-3-yl)-3-methyl-2-oxo-2,3-dihydro-1H-benzo[d]imidazol-5-yl)piperidin-4-yl)acetate (3, 1.2 g, 2.02 mmol) in THF (8 mL) and water (2 mL) was added lithium hydroxide monohydrate (508.21 mg, 12.11 mmol) at rt. The reaction mixture was stirred for 16 h. The volatiles were removed under reduced pressure and the residue was diluted with water (20 mL) and acidified with 1.5N HCl (10 mL). The solid obtained was filtered and triturated with Et₂O (20 mL) to afford 2-(1-(1-(2,6-bis(benzyloxy)pyridin-3-yl)-3-methyl-2-oxo-2,3-dihydro-1H-benzo[d]imidazol-5-yl)piperidin-4-yl)acetic acid (4, 900 mg, 76% yield) as an off-white solid. LCMS (ES+): m/z 579.2[M+H]⁺.

### Step 3: 2-(1-(1-(2,6-dioxopiperidin-3-yl)-3-methyl-2-oxo-2,3-dihydro-1H-benzo[d]imidazol-5-yl)piperidin-4-yl)acetic acid (5)

Into a 100 mL single neck round bottom flask containing a well-stirred solution of 2-(1-(1-(2,6-bis(benzyloxy)pyridin-3-yl)-3-methyl-2-oxo-2,3-dihydro-1H-benzo[d]imidazol-5-yl)piperidin-4-yl)acetic acid (4, 800 mg, 1.37 mmol) in anhydrous DMF (10.0 mL) and 1,4-dioxane (10.0 mL) was added palladium hydroxide on carbon (20 wt. % 50% water) (1.6 g, 11.39 mmol) at rt. The reaction mixture was stirred for 16 h under an atmosphere of hydrogen. The reaction mixture was filtered through Celite and washed with 1,4-dioxane (50 mL). Volatiles were evaporated and the residue was triturated with diethyl ether (10 mL) to afford 2-[1-[1-(2,6-dioxo-3-piperidyl)-3-methyl-2-oxo-benzimidazol-5-yl]-4-piperidyl]acetic acid (5, 300 mg, 44% yield) as a pink solid.

LCMS (ES+): m/z 401.3[M+H]⁺.

### 2-(4-(3-(2,6-dioxopiperidin-3-yl)-1-methyl-1H-indazol-6-yl)-3,5-dimethyl-1H-pyrazol-1-yl)acetic acid (6)

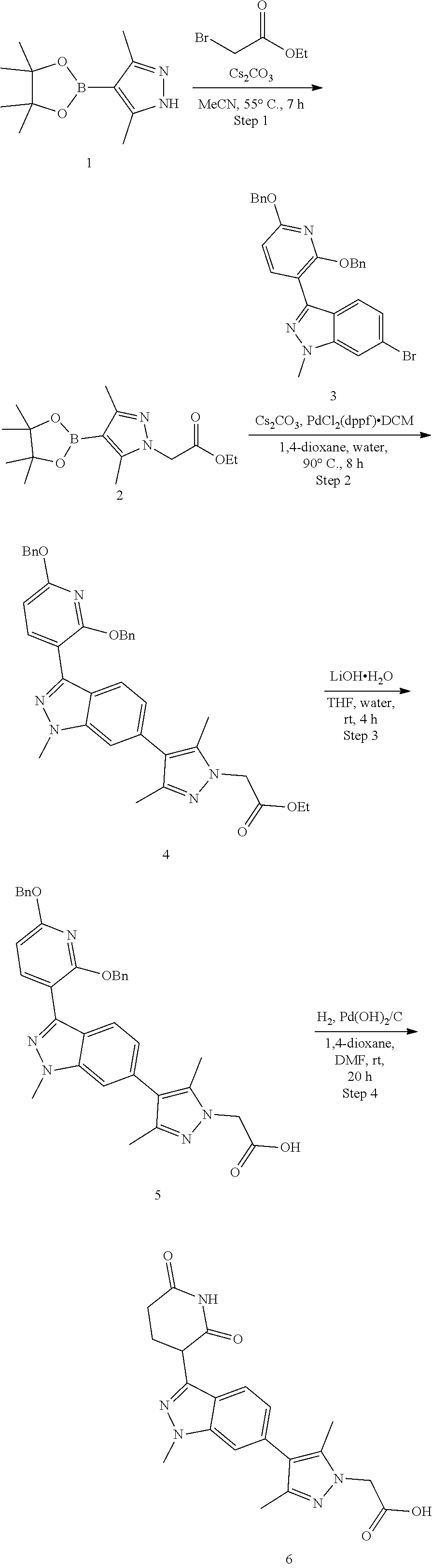

### Step 1: ethyl 2-(3,5-dimethyl-4-(4,4,5,5-tetramethyl-1,3,2-dioxaborolan-2-yl)-1H-pyrazol-1-yl)acetate (2)

Into a 20 mL sealed-tube containing a suspension of 3,5-dimethyl-4-(4,4,5,5-tetramethyl-1,3,2-dioxaborolan-2-yl)-1H-pyrazole (1, 200 mg, 900.53 μmol) and cesium carbonate (440.12 mg, 1.35 mmol) in acetonitrile (1.5 mL) was added dropwise ethyl 2-bromoacetate (165.43 mg, 990.58 μmol, 109.56 μL) in acetonitrile (0.3 mL) and the resulting mixture was heated at 55° C. for 7 h. The reaction mixture was diluted with water (5 mL) and extracted into EtOAc (2×5 mL). The combined organic layers were dried over sodium sulfate and concentrated and purified by silica gel flash column chromatography (230-400 mesh silica gel; 50% EtOAc in pet ether) to obtain ethyl 2-[3,5-dimethyl-4-(4,4,5,5-tetramethyl-1,3,2-dioxaborolan-2-yl)pyrazol-1-yl]acetate (2, 214 mg, 687.46 μmol, 76% yield) as a yellow oil.

LCMS (ES+): m/z 309.2 [M+H]⁺

### Step 2: ethyl 2-(4-(3-(2,6-bis(benzyloxy)pyridin-3-yl)-1-methyl-1H-indazol-6-yl)-3,5-dimethyl-1H-pyrazol-1-yl)acetate (4)

Into a 8 mL screw-capped vial containing a well-stirred solution of ethyl 2-[3,5-dimethyl-4-(4,4,5,5-tetramethyl-1,3,2-dioxaborolan-2-yl)pyrazol-1-yl]acetate (2, 990.49 mg, 3.12 mmol) and 6-bromo-3-(2,6-dibenzyloxy-3-pyridyl)-1-methyl-indazole (3, 1.2 g, 2.40 mmol) in 1,4-dioxane (25 mL) and water (5 mL) was added cesium carbonate (1.56 g, 4.80 mmol) and the resulting mixture was degassed by purging nitrogen gas for five min. To this PdCl₂(dppf)·DCM (293.53 mg, 359.72 μmol) was added and the mixture was heated at 90° C. for 8 h. The reaction mixture was concentrated under vacuum to get the crude material that was purified by silica gel flash column chromatography (230-400 mesh silica gel; 60% EtOAc in Pet ether) to obtain ethyl 2-[4-[3-(2,6-dibenzyloxy-3-pyridyl)-1-methyl-indazol-6-yl]-3,5-dimethyl-pyrazol-1-yl]acetate (4, 1.1 g, 1.81 mmol, 75% yield) as a yellow oil.

LCMS (ES+): m/z 602.8 [M+H]⁺

### Step 3: 2-(4-(3-(2,6-bis(benzyloxy)pyridin-3-yl)-1-methyl-1H-indazol-6-yl)-3,5-dimethyl-1H-pyrazol-1-yl)acetic acid (5)

Into a 50 mL single-neck round-bottom flask containing a well-stirred solution of ethyl 2-[4-[3-(2,6-dibenzyloxy-3-pyridyl)-1-methyl-indazol-6-yl]-3,5-dimethyl-pyrazol-1-yl]acetate (4, 1.1 g, 1.81 mmol) in THF (8 mL) and water (4 mL) was added lithium hydroxide monohydrate (98% (379.75 mg, 9.05 mmol) and the resulting mixture was stirred at room temperature for 4 h. The reaction mixture was concentrated to get the residue that was acidified to pH 1 using 1.5 N HCl and extracted with 10% MeOH in DCM (3×50 mL). The combined organic layers were washed with brine (30 mL), dried over sodium sulfate and evaporated to get the crude material that was triturated with MTBE (3×20 mL) to afford 2-[4-[3-(2,6-dibenzyloxy-3-pyridyl)-1-methyl-indazol-6-yl]-3,5-dimethyl-pyrazol-1-yl]acetic acid (5, 1 g, 1.62 mmol, 90% yield, HCl salt) as a brown solid.

LCMS (ES+): m/z 574.2 [M+H]⁺

### Step 4: 2-(4-(3-(2,6-dioxopiperidin-3-yl)-1-methyl-1H-indazol-6-yl)-3,5-dimethyl-1H-pyrazol-1-yl)acetic acid (6)

Into a 50 mL single-neck round-bottom flask containing a well-stirred solution of 2-[4-[3-(2,6-dibenzyloxy-3-pyridyl)-1-methyl-indazol-6-yl]-3,5-dimethyl-pyrazol-1-yl]acetic acid (5, 1 g, 1.62 mmol, HCl salt) in a mixture of 1,4-dioxane (7 mL) and DMF (4 mL) was added palladium hydroxide on carbon (20 wt. % loading, 1.37 g, 1.95 mmol, 20% purity) and the reaction mixture was stirred at room temperature for 20 h under a hydrogen gas bladder pressure. The reaction mixture was filtered through Celite and washed successively with a mixture of 1,4-dioxane (300 mL) and DMF (300 mL) followed by a mixture of MeCN (200 mL) and THF (200 mL). The filtrate was concentrated, and purified by reverse-phase preparative HPLC [Column: X-Select C18 (250×19) mm, 5 μm; Mobile phase: 0.1% TFA in water and Mobile Phase B: MeCN] to afford 2-[4-[3-(2,6-dioxo-3-piperidyl)-1-methyl-indazol-6-yl]-3,5-dimethyl-pyrazol-1-yl]acetic acid (6, 600 mg, 1.17 mmol, 72% yield, TFA salt) as an off-white solid.

LCMS (ES+): m/z 396.2 [M+H]⁺

### 2-[4-[1-(2,6-dioxo-3-piperidyl)-3-methyl-2-oxo-benzimidazol-5-yl]-3-methyl-pyrazol-1-yl]acetic acid (7a)

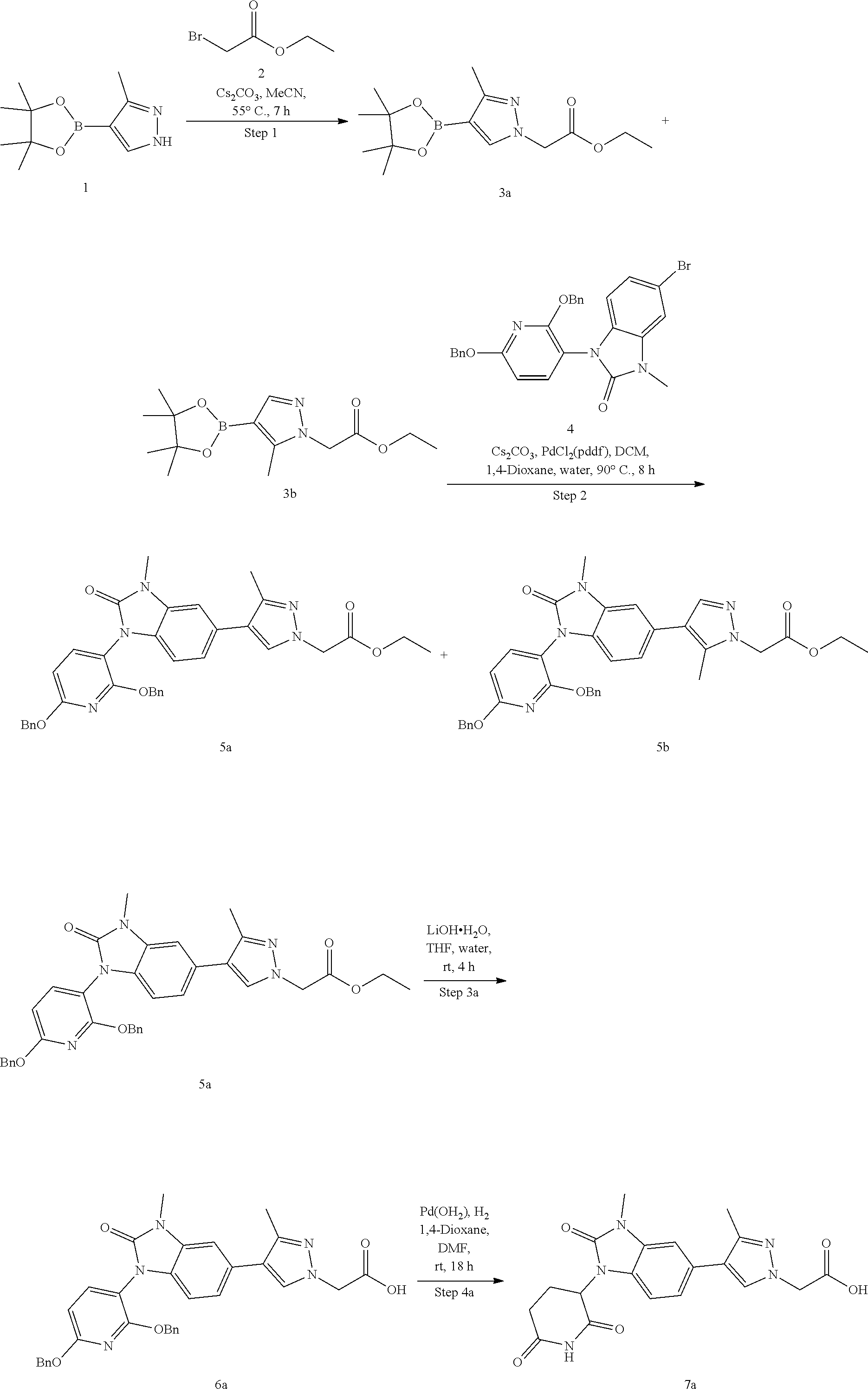

### Step 1: ethyl 2-[3-methyl-4-(4,4,5,5-tetramethyl-1,3,2-dioxaborolan-2-yl)pyrazol-1-yl]acetate (3a) and ethyl 2-[5-methyl-4-(4,4,5,5-tetramethyl-1,3,2-dioxaborolan-2-yl)pyrazol-1-yl]acetate (3b)

Into a 100 mL pressure tube, containing a solution of 3-methyl-4-(4,4,5,5-tetramethyl-1,3,2-dioxaborolan-2-yl)-1H-pyrazole (1, 1.33 g, 6.39 mmol) in acetonitrile (15 mL) was added cesium carbonate (3.12 g, 9.59 mmol) and the suspension was stirred for 5 min. Subsequently, a solution of ethyl 2-bromoacetate (2, 1.28 g, 7.67 mmol, 848.35 μL) in acetonitrile (5 mL) was added dropwise. The reaction mixture was stirred at 55° C. The solvent was removed under reduced pressure and the residue was diluted with EtOAc (50 mL), washed with water (50 mL). The aqueous layer was extracted with EtOAc (2×50 mL). The combined organic layer was dried over sodium sulfate, filtered and evaporated. The crude compound was purified by flash silicagel column chromatography [50% EtOAc in pet ether] to obtain a mixture of ethyl 2-[3-methyl-4-(4,4,5,5-tetramethyl-1,3,2-dioxaborolan-2-yl)pyrazol-1-yl]acetate (3a) and ethyl 2-[5-methyl-4-(4,4,5,5-tetramethyl-1,3,2-dioxaborolan-2-yl)pyrazol-1-yl]acetate (3b) (1.65 g, 5.16 mmol, 81% yield) as a yellow liquid. These compounds could not be separated and were taken forward to the next reaction as a mixture.

LCMS (ES+): m/z 295.2 [M+H]⁺

### Step 2: ethyl 2-[4-[1-(2,6-dibenzyloxy-3-pyridyl)-3-methyl-2-oxo-benzimidazol-5-yl]-3-methyl-pyrazol-1-yl]acetate (5a) and ethyl 2-[4-[1-(2,6-dibenzyloxy-3-pyridyl)-3-methyl-2-oxo-benzimidazol-5-yl]-5-methyl-pyrazol-1-yl]acetate (5b)

Into a 100 mL pressure tube containing a well-stirred solution of 5-bromo-1-(2,6-dibenzyloxy-3-pyridyl)-3-methyl-benzimidazol-2-one (4, 1.2 g, 2.21 mmol), mixture of ethyl 2-[3-methyl-4-(4,4,5,5-tetramethyl-1,3,2-dioxaborolan-2-yl)pyrazol-1-yl]acetate and ethyl 2-[5-methyl-4-(4,4,5,5-tetramethyl-1,3,2-dioxaborolan-2-yl)pyrazol-1-yl]acetate (3a and 3b, 917.62 mg, 2.87 mmol) in 1,4-dioxane (25 mL) and water (5 mL) was added cesium carbonate (1.80 g, 5.52 mmol). The mixture was degassed by purging nitrogen gas for 10 min. Then, PdCl₂(dppf)DCM (270.22 mg, 331.15 μmol) was added and the reaction mixture was stirred at 90° C. After 8 h, the reaction mixture was filtered through Celite, washed with EtOAc (100 mL). The filtrate was dried over sodium sulfate and purified by flash silicagel flash column chromatography (60% EtOAc in pet ether) to obtain (550 mg) of the product that was subjected to SFC purification to separate regioisomers.

Chiral SFC method: Column Name: Chiralpak AS-H; Co-solvent: 45% and Co-solvent Name: 0.1% Isopropyl Amine in IPA: MeCN (1:1); Outlet Pressure: 100 bar; Temperature: 35° C. After the separation the fast eluted regioisomer ethyl 2-[4-[1-(2,6-dibenzyloxy-3-pyridyl)-3-methyl-2-oxo-benzimidazol-5-yl]-3-methyl-pyrazol-1-yl]acetate (5a, 260 mg, 409.17 μmol, 19% yield) (RT: 1.23) was obtained as pale brown sticky solid.

LCMS (ES+): m/z 604.2 [M+H]⁺

And the late eluted regioisomer ethyl 2-[4-[1-(2,6-dibenzyloxy-3-pyridyl)-3-methyl-2-oxo-benzimidazol-5-yl]-5-methyl-pyrazol-1-yl]acetate (5b, 250 mg, 397.57 μmol, 18% yield) (RT: 1.59) was obtained as a pale brown sticky solid.

LCMS (ES+): m/z 604.2 [M+H]⁺

### Step 3a: 2-[4-[1-(2,6-dibenzyloxy-3-pyridyl)-3-methyl-2-oxo-benzimidazol-5-yl]-3-methyl-pyrazol-1-yl]acetic acid (6a)

Into a 50 mL single-neck round-bottom flask containing a well-stirred solution of ethyl 2-[4-[1-(2,6-dibenzyloxy-3-pyridyl)-3-methyl-2-oxo-benzimidazol-5-yl]-3-methyl-pyrazol-1-yl]acetate (5a, 250 mg, 393.43 μmol) in THF (3 mL), was added a solution of lithium hydroxide monohydrate (49.53 mg, 1.18 mmol) in water (0.5 mL) and stirred the reaction mixture at ambient temperature for 4 h. The solvent was removed under reduced pressure and the residue was acidified using aq. 1.5 N HCl (2 mL) and the solid precipitated was collected by filtration, washed with water and dried under reduced pressure to obtain 2-[4-[1-(2,6-dibenzyloxy-3-pyridyl)-3-methyl-2-oxo-benzimidazol-5-yl]-3-methyl-pyrazol-1-yl]acetic acid (6a, 240 mg, 384.27 μmol, 98% yield, HCl salt) as a brown solid.

LCMS (ES+): m/z 576.2 [M+H]⁺

### Step 4a: 2-[4-[1-(2,6-dioxo-3-piperidyl)-3-methyl-2-oxo-benzimidazol-5-yl]-3-methyl-pyrazol-1-yl]acetic acid (a)

To a 50 mL single-neck round-bottom flask containing a suspension of 2-[4-[1-(2,6-dibenzyloxy-3-pyridyl)-3-methyl-2-oxo-benzimidazol-5-yl]-3-methyl-pyrazol-1-yl]acetic acid (6a, 240 mg, 384.27 μmol, HCl salt) in a mixture of 1,4-dioxane (2 mL), DMF (2 mL), was added 20 wt. % palladium hydroxide on carbon (296.80 mg, 422.69 μmol, 20% purity). The resulting suspension was stirred at ambient temperature under hydrogen atmosphere (bladder). After 18 h, the reaction mixture was filtered through Celite and washed with a mixture of 1,4-dioxane (150 mL), DMF (150 mL), followed by a mixture of MeCN (100 mL) and THF (100 mL). The combined filtrate was concentrated to dryness under reduced pressure to obtain 2-[4-[1-(2,6-dioxo-3-piperidyl)-3-methyl-2-oxo-benzimidazol-5-yl]-3-methyl-pyrazol-1-yl]acetic acid (7a, 150 mg, 354.82 μmol, 92% yield) as a red solid.

LCMS (ES−): m/z 396 [M−H]⁻

### 2-[4-[[1-(2,6-dioxo-3-piperidyl)-3-methyl-2-oxo-benzimidazol-5-yl]methyl]piperazin-1-yl]acetic acid (4)

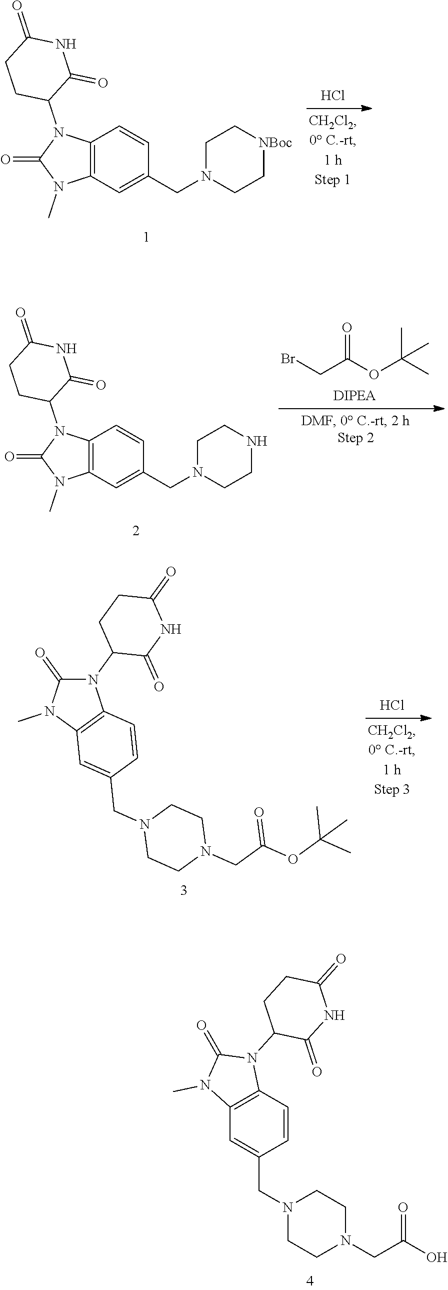

### Step 1: 3-[3-methyl-2-oxo-5-(piperazin-1-ylmethyl)benzimidazol-1-yl]piperidine-2,6-dione (2)

In to a 50 mL single-neck round-bottom flask containing a well-stirred solution of tert-butyl 4-[[1-(2,6-dioxo-3-piperidyl)-3-methyl-2-oxo-benzimidazol-5-yl]methyl]piperazine-1-carboxylate (1, 220 mg, 456.81 μmol) in anhydrous DCM (1 mL) was added dropwise HCl (4.0 M in 1,4-dioxane, 6.60 mmol, 1.5 mL) at 0° C. under nitrogen atmosphere. The resulting mixture was stirred at rt for 1 h. The reaction mixture was evaporated to dryness and the crude material was triturated with Et₂O (2×10 mL) to afford 3-[3-methyl-2-oxo-5-(piperazin-1-ylmethyl)benzimidazol-1-yl]piperidine-2,6-dione (2, 180 mg, 429.59 μmol, 94% yield, HCl salt) as an off-white solid.

LCMS (ES+): m/z 358.2 [M+H]⁺

### Step 2: tert-butyl 2-[4-[[1-(2,6-dioxo-3-piperidyl)-3-methyl-2-oxo-benzimidazol-5-yl]methyl]piperazin-1-yl]acetate (3)

In to a 25 mL single-neck round-bottom flask containing a well-stirred solution of 3-[3-methyl-2-oxo-5-(piperazin-1-ylmethyl)benzimidazol-1-yl]piperidine-2,6-dione (2, 180 mg, 429.59 μmol, HCl salt) in anhydrous DMF (2.5 mL) was added DIPEA (277.61 mg, 2.15 mmol, 374.13 μL) followed by tert-butyl 2-bromoacetate (92.17 mg, 472.55 μmol, 69.30 μL) at 0° C. under nitrogen atmosphere. The resulting mixture was stirred at rt for 2 h. The reaction mixture was diluted with water (5 mL) and extracted with EtOAc (2×20 mL). The organic layers were combined, dried over Na₂SO₄ and concentrated to afford tert-butyl 2-[4-[[1-(2,6-dioxo-3-piperidyl)-3-methyl-2-oxo-benzimidazol-5-yl]methyl]piperazin-1-yl]acetate (3, 210 mg, 351.82 μmol, 82% yield) as a brown oil.

LCMS (ES+): m/z 472.3 [M+H]⁺

### Step 3: 2-[4-[[1-(2,6-dioxo-3-piperidyl)-3-methyl-2-oxo-benzimidazol-5-yl]methyl]piperazin-1-yl]acetic acid (4)

In to a 50 mL single-neck round-bottom flask containing a well-stirred solution of tert-butyl 2-[4-[[1-(2,6-dioxo-3-piperidyl)-3-methyl-2-oxo-benzimidazol-5-yl]methyl]piperazin-1-yl]acetate (3, 210 mg, 351.82 μmol) in anhydrous DCM (2 mL) was added dropwise HCl (4 M in 1,4-dioxane, 6.16 mmol, 1.5 mL) at 0° C. under nitrogen atmosphere. The resulting mixture was stirred at rt for 1 h. The reaction mixture was evaporated to dryness and the crude material was triturated with Et₂O (2×10 mL) to afford 2-[4-[[1-(2,6-dioxo-3-piperidyl)-3-methyl-2-oxo-benzimidazol-5-yl]methyl]piperazin-1-yl]acetic acid (4, 190 mg, 344.76 μmol, 98% yield, HCl salt) as an off-white solid.

LCMS (ES+): m/z 416.2 [M+H]⁺

### 2-(5-(1-(2,6-dioxopiperidin-3-yl)-3-methyl-2-oxo-2,3-dihydro-1H-benzo[d]imidazol-5-yl)-2,5-diazabicyclo[2.2.2]octan-2-yl)acetic acid (7)

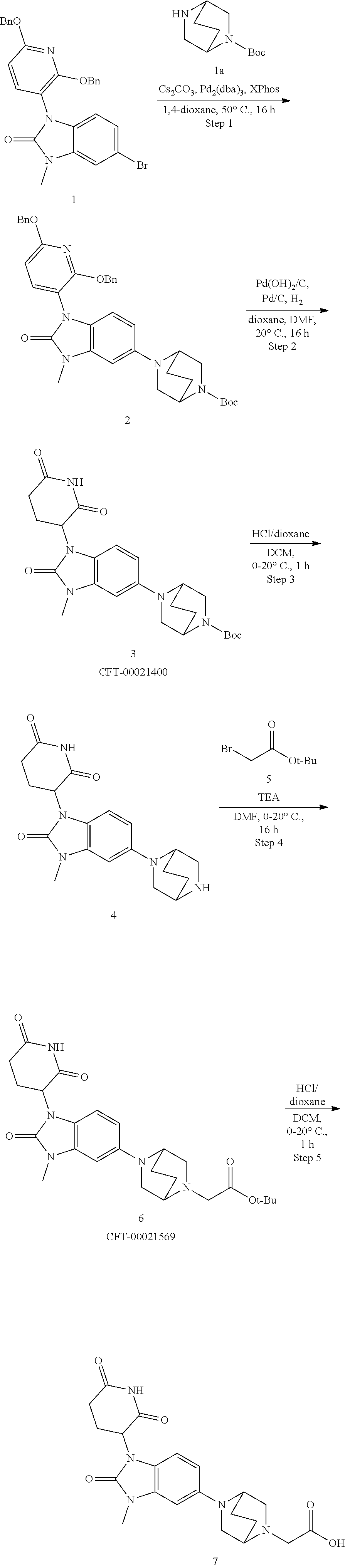

### Step 1: tert-butyl 5-(1-(2,6-bis(benzyloxy)pyridin-3-yl)-3-methyl-2-oxo-2,3-dihydro-1H-benzo[d]imidazol-5-yl)-2,5-diazabicyclo[2.2.2]octane-2-carboxylate (2)

To a mixture of 1-(2,6-bis(benzyloxy)pyridin-3-yl)-5-bromo-3-methyl-1H-benzo[d]imidazol-2 (3H)-one (1, 1 g, 1.94 mmol, 1 eq), tert-butyl 2,5-diazabicyclo[2.2.2]octane-2-carboxylate (1a, 411.10 mg, 1.94 mmol, 1 eq), Cs₂CO₃ (1.26 g, 3.87 mmol, 2 eq) in 1,4-dioxane (20 mL) was added Pd₂(dba)₃ (177.33 mg, 193.65 μmol, 0.1 eq) and Xphos (184.64 mg, 387.31 μmol, 0.2 eq) under N₂, the mixture was stirred at 90° C. for 16 h. The mixture was purified column chromatography (SiO₂, Petroleum ether/Ethyl acetate=100/1 to 1/1) to afford tert-butyl 5-(1-(2,6-bis(benzyloxy)pyridin-3-yl)-3-methyl-2-oxo-2,3-dihydro-1H-benzo[d]imidazol-5-yl)-2,5-diazabicyclo[2.2.2]octane-2-carboxylate (2, 950 mg, 1.45 mmol, 75% yield) as yellow solid.

LCMS (ESI): m/z 648.5 [M+H]⁺

### Step 2: tert-butyl 5-(1-(2,6-dioxopiperidin-3-yl)-3-methyl-2-oxo-2,3-dihydro-1H-benzo[d]imidazol-5-yl)-2,5-diazabicyclo[2.2.2]octane-2-carboxylate (3)

To a solution of tert-butyl 5-(1-(2,6-bis(benzyloxy)pyridin-3-yl)-3-methyl-2-oxo-2,3-dihydro-1H-benzo[d]imidazol-5-yl)-2,5-diazabicyclo[2.2.2]octane-2-carboxylate (2, 500 mg, 771.89 μmol, 1 eq) in 1.4-dioxane (5 mL) and DMF (5 mL) was added Pd/C (25 mg, 10% purity) and Pd(OH)₂/C (25 mg, 10% purity) under H₂, then the mixture was stirred at 20° C. for 16 h under H₂ (15 psi) atmosphere. The reaction mixture was filtered through Celite. The filtrate was poured into H₂O (10 mL). The mixture was extracted with EtOAc (5 mL×2). The combined organic was washed with brine (10 mL), dried with anhydrous Na₂SO₄, filtered and concentrated in vacuum. The residue was purified by prep-HPLC (flow: 25 mL/min; gradient: from 37-67% MeCN in water (0.1% TFA); column: 3_Phenomenex Luna C18 75×30 mm×3 um) to afford tert-butyl 5-(1-(2,6-dioxopiperidin-3-yl)-3-methyl-2-oxo-2,3-dihydro-1H-benzo[d]imidazol-5-yl)-2,5-diazabicyclo[2.2.2]octane-2-carboxylate (3, 340 mg, 582.64 μmol, 75% yield, TFA salt).

LCMS (ESI): m/z 470.0 [M+H]⁺

¹H NMR (400 MHz, d₆-DMSO) δ 11.04 (s, 1H), 6.90 (d, J=8.8 Hz, 1H), 6.57 (d, J=1.8 Hz, 1H), 6.35 (br d, J=8.8 Hz, 1H), 5.26 (dd, J=5.4, 12.8 Hz, 1H), 4.21-4.11 (m, 2H), 3.38-3.32 (m, 2H), 3.29 (s, 3H), 2.93-2.84 (m, 1H), 2.72-2.59 (m, 3H), 2.11-1.65 (m, 6H), 1.40 (d, J=5.4 Hz, 9H).

### Step 3: 3-(5-(2,5-diazabicyclo[2.2.2]octan-2-yl)-3-methyl-2-oxo-2,3-dihydro-1H-benzo[d]imidazol-1-yl)piperidine-2,6-dione (4)

To a solution of tert-butyl 5-(1-(2,6-dioxopiperidin-3-yl)-3-methyl-2-oxo-2,3-dihydro-1H-benzo[d]imidazol-5-yl)-2,5-diazabicyclo[2.2.2]octane-2-carboxylate (3, 130 mg, 276.87 μmol, 1 eq) in DCM (2 mL) was added HCl/dioxane (4 M, 1 mL) at 0° C., then the mixture was stirred at 20° C. for 1 h. The mixture was concentrated in vacuum to afford 3-(5-(2,5-diazabicyclo[2.2.2]octan-2-yl)-3-methyl-2-oxo-2,3-dihydro-1H-benzo[d]imidazol-1-yl)piperidine-2,6-dione (4, 112 mg, 275.95 μmol, 100% yield, HCl salt) as yellow solid. The material was used in the next step without further purification.

LCMS (ESI): m/z 369.9 [M+H]⁺

### Step 4: tert-butyl 2-(5-(1-(2,6-dioxopiperidin-3-yl)-3-methyl-2-oxo-2,3-dihydro-1H-benzo[d]imidazol-5-yl)-2,5-diazabicyclo[2.2.2]octan-2-yl)acetate (6)

To a mixture of 3-(5-(2,5-diazabicyclo[2.2.2]octan-2-yl)-3-methyl-2-oxo-2,3-dihydro-1H-benzo[d]imidazol-1-yl)piperidine-2,6-dione (4, 112 mg, 275.95 μmol, 1 eq, HCl salt) and TEA (83.77 mg, 827.84 μmol, 115.38 μL, 3 eq) in DMF (3 mL) was added tert-butyl 2-bromoacetate (5, 53.82 mg, 275.95 μmol, 40.47 μL, 1 eq) at 0° C., then the mixture was stirred at 20° C. for 16 h. The mixture was poured into H₂O (10 mL). The mixture was extracted with EtOAc (5 mL×2). The combined organic was washed with brine (10 mL), dried with anhydrous Na₂SO₄, filtered and concentrated in vacuum. The residue was purified by prep-HPLC (flow: 25 mL/min; gradient: from 24-54% MeCN in water (10 mM NH₄HCO₃); column: Waters Xbridge 150×25 mm×5 um) to afford tert-butyl 2-(5-(1-(2,6-dioxopiperidin-3-yl)-3-methyl-2-oxo-2,3-dihydro-1H-benzo[d]imidazol-5-yl)-2,5-diazabicyclo[2.2.2]octan-2-yl)acetate (6, 94 mg, 194.39 μmol, 70% yield) as white solid.

LCMS (ESI): m/z 484.5 [M+H]⁺

¹H NMR (400 MHz, d₆-DMSO) δ 11.18-10.84 (m, 1H), 6.88 (br d, J=8.6 Hz, 1H), 6.51 (s, 1H), 6.31 (br d, J=8.8 Hz, 1H), 5.24 (br dd, J=4.8, 12.8 Hz, 1H), 3.86 (br s, 1H), 3.60 (br d, J=9.8 Hz, 1H), 3.28 (s, 3H), 3.21-3.15 (m, 2H), 2.99 (br d, J=9.8 Hz, 1H), 2.91-2.83 (m, 3H), 2.68-2.59 (m, 2H), 2.03-1.68 (m, 5H), 1.60-1.52 (m, 1H), 1.40 (s, 9H).

### Step 5: 2-(5-(1-(2,6-dioxopiperidin-3-yl)-3-methyl-2-oxo-2,3-dihydro-1H-benzo[d]imidazol-5-yl)-2,5-diazabicyclo[2.2.2]octan-2-yl)acetic acid (7)

To a solution of tert-butyl 2-(5-(1-(2,6-dioxopiperidin-3-yl)-3-methyl-2-oxo-2,3-dihydro-1H-benzo[d]imidazol-5-yl)-2,5-diazabicyclo[2.2.2]octan-2-yl)acetate (6, 140 mg, 289.52 μmol, 1 eq) in DCM (4 mL) was added HCl/dioxane (4 M, 2 mL) at 0° C., then the mixture was stirred at 20° C. for 1 h. The mixture was concentrated in vacuum to afford 2-(5-(1-(2,6-dioxopiperidin-3-yl)-3-methyl-2-oxo-2,3-dihydro-1H-benzo[d]imidazol-5-yl)-2,5-diazabicyclo[2.2.2]octan-2-yl)acetic acid (7, 134 mg, 288.85 μmol, 100% yield, HCl salt) as white solid. The material was used in the next step without further purification.

LCMS (ESI): m/z 427.9 [M+H]⁺

### 3-(1-(1-(2,6-dioxopiperidin-3-yl)-3-methyl-2-oxo-2,3-dihydro-1H-benzo[d]imidazol-5-yl) piperidin-4-yl) propionic acid (2)

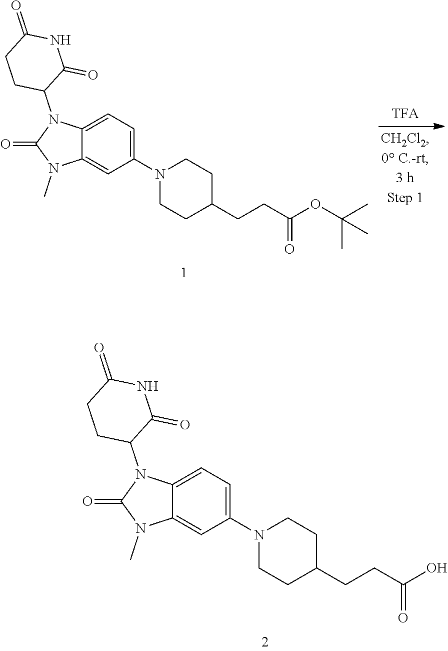 [SMILES: Cn1c(=O)n(C2CCC(=O)NC2=O)c2ccc(N3CCC(CCC(=O)OC(C)(C)C)CC3)cc21 ; "TFA" ; Cn1c(=O)n(C2CCC(=O)NC2=O)c2ccc(N3CCC(CCC(=O)O)CC3)cc21]

### Step 1: 3-(1-(1-(2,6-dioxopiperidin-3-yl)-3-methyl-2-oxo-2,3-dihydro-1H-benzo[d]imidazol-5-yl) piperidin-4-yl) propionic acid (2)

In to a 100 mL single-neck, round-bottom flask containing a well-stirred solution of tert-butyl 3-(1-(1-(2,6-dioxopiperidin-3-yl)-3-methyl-2-oxo-2,3-dihydro-1H-benzo[d]imidazol-5-yl) piperidin-4-yl) propionate (1, 0.3 g, 637.54 μmol) in anhydrous DCM (10 mL) was added TFA (1.82 g, 15.94 mmol, 1.23 mL) dropwise at 0° C. The resulting mixture was stirred at room temperature for 3 h. The reaction mixture was concentrated to dryness and the crude material was triturated with diethyl ether to get 3-[1-[1-(2,6-dioxo-3-piperidyl)-3-methyl-2-oxo-benzimidazol-5-yl]-4-piperidyl] propionic acid (2, 0.3 g, 535.26 μmol, 84% yield, TFA salt) as a gummy syrup.

LCMS (ES+): m/z 415.2 [M+H]⁺

### 2-(4-(1-(2,6-bis(benzyloxy)pyridin-3-yl)-3-(oxetan-3-yl)-2-oxo-2,3-dihydro-1H-benzo[d]imidazol-5-yl)-5,6-dihydropyridin-1 (2H)-yl)acetic acid (7)

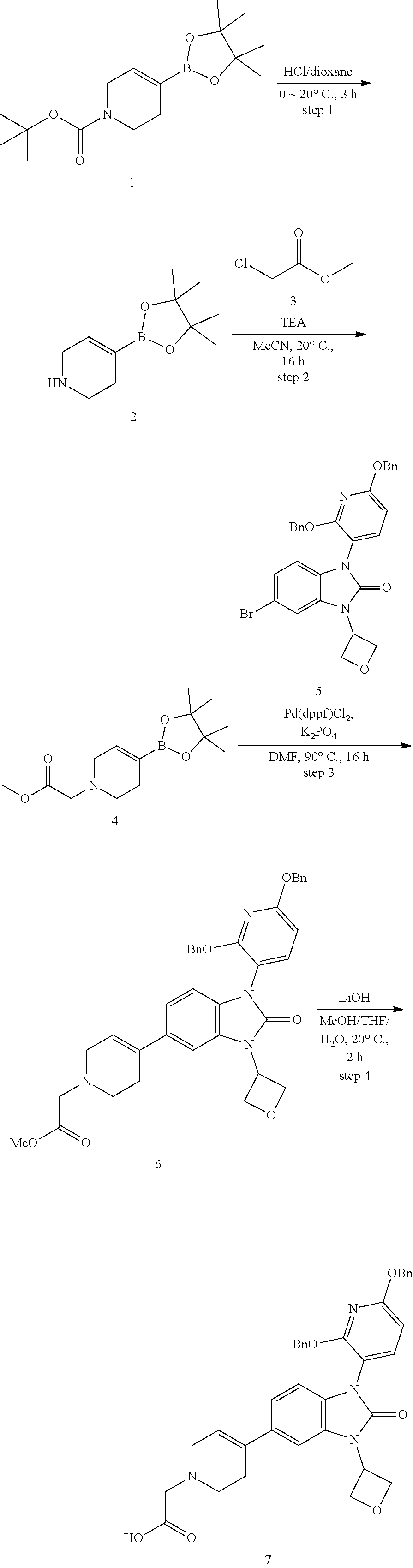

### Step 1: 4-(4,4,5,5-tetramethyl-1,3,2-dioxaborolan-2-yl)-1,2,3,6-tetrahydropyridine (2)

A mixture of tert-butyl 4-(4,4,5,5-tetramethyl-1,3,2-dioxaborolan-2-yl)-5,6-dihydropyridine-1 (2H)-carboxylate (1, 3 g, 9.70 mmol, 1 eq) in HCl/dioxane (4 M, 30.00 mL) was stirred at 0˜20° C. for 3 h. The reaction mixture was concentrated under reduced pressure to afford 4-(4,4,5,5-tetramethyl-1,3,2-dioxaborolan-2-yl)-1,2,3,6-tetrahydropyridine (2, 2.4 g, 9.68 mmol, 99% yield, HCl salt) as an off-white solid. The material was used in the next step without further purification.

LCMS (ESI): m/z 210.2 [M+H]⁺

### Step 2: methyl 2-(4-(4,4,5,5-tetramethyl-1,3,2-dioxaborolan-2-yl)-5,6-dihydropyridin-1 (2H)-yl)acetate (4)

To a solution of 4-(4,4,5,5-tetramethyl-1,3,2-dioxaborolan-2-yl)-1,2,3,6-tetrahydropyridine (2, 1 g, 4.07 mmol, HCl salt, 1 eq) and methyl 2-chloroacetate (3, 530.35 mg, 4.89 mmol, 427.70 μL, 1.2 eq) in MeCN (10 mL) was added TEA (1.03 g, 10.18 mmol, 1.42 mL, 2.5 eq). The mixture was stirred at 20° C. for 16 h. The reaction mixture was purified by flash silica gel chromatography (60 mL/min, Eluent of 0-70% ethyl acetate/petroleum ether gradient, Column: Bigtage®; 12 g SepaFlash® Silica Flash) to afford methyl 2-(4-(4,4,5,5-tetramethyl-1,3,2-dioxaborolan-2-yl)-5,6-dihydropyridin-1 (2H)-yl)acetate (4, 720 mg, 2.51 mmol, 62% yield) as a white solid.

LCMS (ESI): m/z 282.2 [M+H]⁺

### Step 3: methyl 2-(4-(1-(2,6-bis(benzyloxy)pyridin-3-yl)-3-(oxetan-3-yl)-2-oxo-2,3-dihydro-1H-benzo[d]imidazol-5-yl)-5,6-dihydropyridin-1 (2H)-yl)acetate (6)

To a solution of methyl 2-(4-(4,4,5,5-tetramethyl-1,3,2-dioxaborolan-2-yl)-5,6-dihydropyridin-1 (2H)-yl)acetate (4, 1 g, 3.56 mmol, 1.2 eq) and 1-(2,6-bis(benzyloxy)pyridin-3-yl)-5-bromo-3-(oxetan-3-yl)-1H-benzo[d]imidazol-2 (3H)-one (5, 1.66 g, 2.96 mmol, 1 eq) in DMF (15 mL) were added K₃PO₄ (1.89 g, 8.89 mmol, 3 eq) and Pd(dppf)Cl₂ (108.44 mg, 148.20 μmol, 0.05 eq). The mixture was degassed and purged with N₂ for 3 times. Then the mixture was stirred at 90° C. for 16 h under N₂ atmosphere. The reaction mixture was diluted with water (40 mL) and extracted with ethyl acetate (40 mL×3). The combined organic layers were washed with brine (80 mL), dried over Na₂SO₄, filtered and concentrated under reduced pressure to give residue. The residue was purified by flash silica gel chromatography (60 mL/min, Eluent of 0-100% ethyl acetate/petroleum ether gradient, Column: Bigtage®; 12 g SepaFlash® Silica Flash) to afford methyl 2-(4-(1-(2,6-bis(benzyloxy)pyridin-3-yl)-3-(oxetan-3-yl)-2-oxo-2,3-dihydro-1H-benzo[d]imidazol-5-yl)-5,6-dihydropyridin-1 (2H)-yl)acetate (6, 1.1 g, 1.39 mmol, 47% yield) as a yellow solid.

LCMS (ESI): m/z 633.3 [M+H]⁺

### Step 4: 2-(4-(1-(2,6-bis(benzyloxy)pyridin-3-yl)-3-(oxetan-3-yl)-2-oxo-2,3-dihydro-1H-benzo[d]imidazol-5-yl)-5,6-dihydropyridin-1 (2H)-yl)acetic acid (7)

To a solution of methyl 2-(4-(1-(2,6-bis(benzyloxy)pyridin-3-yl)-3-(oxetan-3-yl)-2-oxo-2,3-dihydro-1H-benzo[d]imidazol-5-yl)-5,6-dihydropyridin-1 (2H)-yl)acetate (6, 500 mg, 790.26 μmol, 1 eq) in THF (2 mL) and Methanol (2 mL) were added LiOH·H₂O (165.81 mg, 3.95 mmol, 109.81 μL, 5 eq) and H₂O (790.26 μmol, 2 mL). The mixture was stirred at 20° C. for 2 h. The reaction mixture was acidified to pH=5 with 1 N aqueous HCl, then the mixture was diluted with water (10 mL) and extracted with ethyl acetate (10 mL×3). The combined organic layers were washed with brine (20 mL), dried over Na₂SO₄, filtered and concentrated under reduced pressure to give residue. The residue was purified by flash silica gel chromatography (60 mL/min, Eluent of 50-100% ethyl acetate/petroleum ether gradient and 83% ethyl acetate/ethanol gradient, Column: Bigtage®; 4 g SepaFlash® Silica Flash) to afford 2-(4-(1-(2,6-bis(benzyloxy)pyridin-3-yl)-3-(oxetan-3-yl)-2-oxo-2,3-dihydro-1H-benzo[d]imidazol-5-yl)-5,6-dihydropyridin-1 (2H)-yl)acetic acid (7, 450 mg, 669.17 μmol, 85% yield) as a green solid.

LCMS (ESI): m/z 619.4 [M+H]⁺

### 2-[4-[3-(2,6-dioxo-3-piperidyl)-1-ethyl-indazol-6-yl]-3,3-difluoro-1-piperidyl]acetic acid (4)

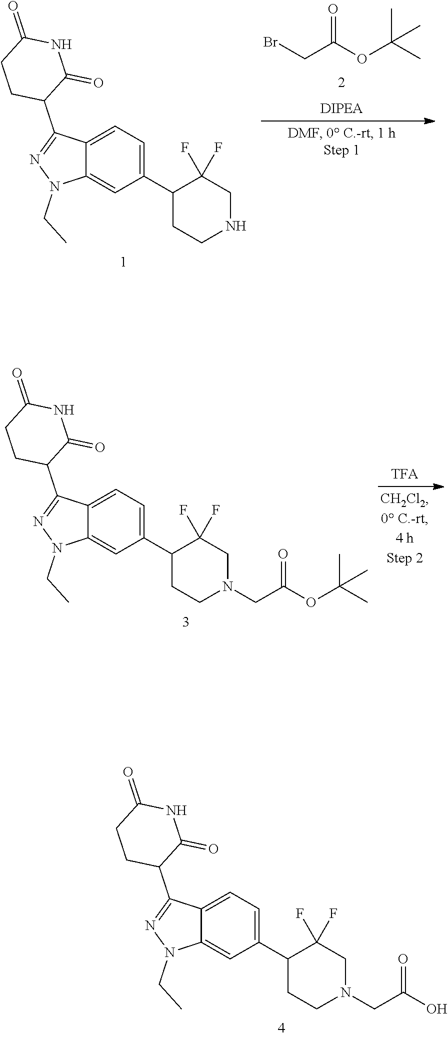

### Step 1: tert-butyl 2-[4-[3-(2,6-dioxo-3-piperidyl)-1-ethyl-indazol-6-yl]-3,3-difluoro-1-piperidyl]acetate (3)

Into a 100 mL single-neck, round-bottom flask containing a well-stirred solution of 3-[6-(3,3-difluoro-4-piperidyl)-1-ethyl-indazol-3-yl]piperidine-2,6-dione (1, 400 mg, 747.11 μmol, TFA salt) in anhydrous DMF (10 mL) were added DIPEA (482.79 mg, 3.74 mmol, 650.67 μL) and tert-butyl 2-bromoacetate (2, 218.59 mg, 1.12 mmol, 164.35 μL) at 0° C. The resulting mixture was stirred at room temperature for 1 h. The reaction mixture was concentrated and diluted with water (80 mL) to precipitate a solid that was filtered and dried under reduced pressure to afford tert-butyl 2-[4-[3-(2,6-dioxo-3-piperidyl)-1-ethyl-indazol-6-yl]-3,3-difluoro-1-piperidyl]acetate (3, 380 mg, 719.42 μmol, 96% yield) as an off-white solid.

LCMS (ES+): m/z 491.2 [M+H]⁺

### Step 2: 2-[4-[3-(2,6-dioxo-3-piperidyl)-1-ethyl-indazol-6-yl]-3,3-difluoro-1-piperidyl]acetic acid (4)

Into a 100 mL single-neck, round-bottom flask containing a well-stirred solution of tert-butyl 2-[4-[3-(2,6-dioxo-3-piperidyl)-1-ethyl-indazol-6-yl]-3,3-difluoro-1-piperidyl]acetate (3, 380 mg, 719.42 μmol) in anhydrous DCM (6 mL) was added TFA (2.96 g, 25.96 mmol, 2 mL) at room temperature. The reaction mixture was stirred at room temperature for 4 h. Thereafter, solvent was evaporated to get the crude compound, which upon trituration with MTBE (70 mL) afforded 2-[4-[3-(2,6-dioxo-3-piperidyl)-1-ethyl-indazol-6-yl]-3,3-difluoro-1-piperidyl]acetic acid (4, 310 mg, 519.83 μmol, 72% yield, TFA salt) as an off-white solid.

LCMS (ES+): m/z 435.2 [M+H]⁺

### 2-[4-[3-(2,6-dioxo-3-piperidyl)-1-isopropyl-indazol-6-yl]-3,3-difluoro-1-piperidyl]acetic acid (4)

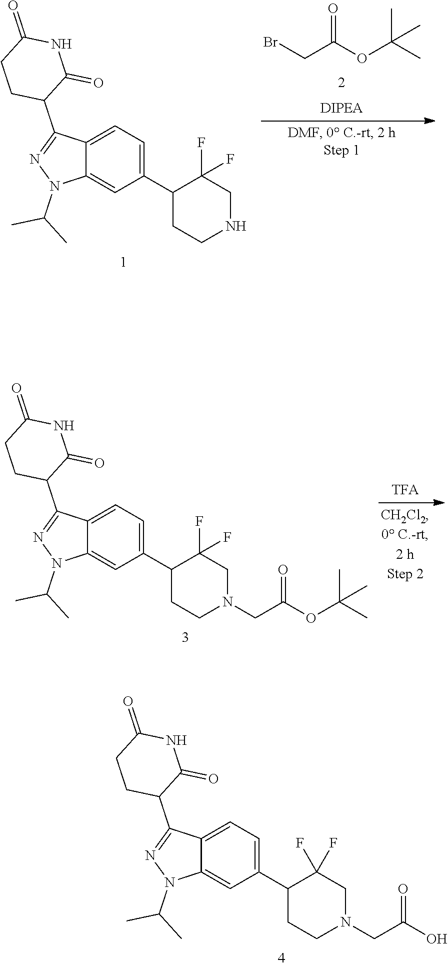

### Step 1: tert-butyl 2-[4-[3-(2,6-dioxo-3-piperidyl)-1-isopropyl-indazol-6-yl]-3,3-difluoro-1-piperidyl]acetate (3)

Into a 50 mL two-neck, round-bottom flask containing a well-stirred solution of 3-[6-(3,3-difluoro-4-piperidyl)-1-isopropyl-indazol-3-yl]piperidine-2,6-dione (1, 600 mg, 1.12 mmol, TFA salt) in DMF (10 mL) was added Et₃N (565.68 mg, 5.59 mmol, 779.17 μL) and tert-butyl 2-bromoacetate (2, 261.70 mg, 1.34 mmol, 196.76 μL) under nitrogen atmosphere. The reaction mixture was stirred at room temperature for 2 h. The reaction mixture was diluted with water (100 mL) and extracted twice with EtOAc (2×100 mL). the combined organic phases were washed with brine, dried over anhydrous Na₂SO₄, filtered and concentrated under reduced pressure to get product tert-butyl 2-[4-[3-(2,6-dioxo-3-piperidyl)-1-isopropyl-indazol-6-yl]-3,3-difluoro-1-piperidyl]acetate (3, 450 mg, 847.26 μmol, 76% yield) which was taken as such for the next step without further purification.

LCMS (ES+): m/z 505.2 [M+H]⁺

### Step 2: 2-[4-[3-(2,6-dioxo-3-piperidyl)-1-isopropyl-indazol-6-yl]-3,3-difluoro-1-piperidyl]acetic acid (4)

Into a 100 mL single-neck, round-bottom flask containing a well-stirred solution of tert-butyl 2-[4-[3-(2,6-dioxo-3-piperidyl)-1-isopropyl-indazol-6-yl]-3,3-difluoro-1-piperidyl]acetate (450 mg, 847.26 μmol) in anhydrous DCM (10 mL) was added TFA (7.40 g, 64.90 mmol, 5 mL) at 0° C. under nitrogen atmosphere. The resulting mixture was stirred at ambient temperature for 2 h. The reaction mixture was concentrated under reduced pressure to get the crude material that was triturated with MTBE to afford 2-[4-[3-(2,6-dioxo-3-piperidyl)-1-isopropyl-indazol-6-yl]-3,3-difluoro-1-piperidyl]acetic acid (4, 400 mg, 520.32 μmol, 61% yield, TFA salt) as an off-white solid.

LCMS (ES+): m/z 449.2 [M+H]⁺

### 2-[[4-[1-(2,6-dioxo-3-piperidyl)-3-methyl-2-oxo-benzimidazol-5-yl]-1-piperidyl]methyl]cyclopropanecarboxylic acid (3)

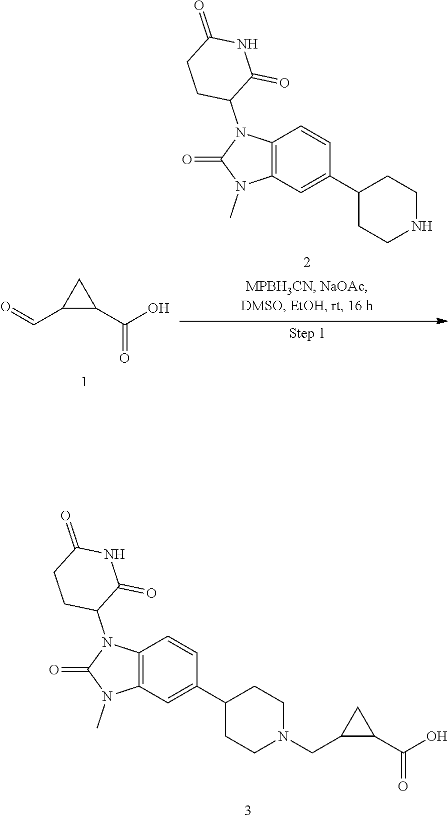 [SMILES: Cn1c(=O)n(C2CCC(=O)NC2=O)c2ccc(C3CCNCC3)cc21 ; O=CC1CC1C(=O)O ; Cn1c(=O)n(C2CCC(=O)NC2=O)c2ccc(C3CCN(CC4CC4C(=O)O)CC3)cc21]

### Step 2: 2-[[4-[1-(2,6-dioxo-3-piperidyl)-3-methyl-2-oxo-benzimidazol-5-yl]-1-piperidyl]methyl]cyclopropanecarboxylic acid (4)

Into a 50 mL single neck round bottom flask containing a well-stirred solution of 3-[3-methyl-2-oxo-5-(4-piperidyl)benzimidazol-1-yl]piperidine-2,6-dione (2, 200 mg, 438.20 μmol, TFA salt) and 2-formylcyclopropanecarboxylic acid (1, 52.08 mg, 438.20 μmol) in DMSO (2 mL) and ethanol (2 mL) was added anhydrous sodium acetate (107.84 mg, 1.31 mmol) and acetic acid (263.14 mg, 4.38 mmol). Afterwards the suspension was stirred for 10 min and Biotage® MP-Cyanoborohydride (2 mmol in 1 g) (400 mg, 800.40 μmol) was added and the reaction mixture was stirred at room temperature. After 16 h, the reaction mixture was filtered through Celite and washed with ethanol (20 mL). The filtrate was concentrated to dryness under reduced pressure and the residue was subjected to reverse phase column chromatography [Purification method: Siliasep premium C18, 25 um, 120 g, Mobile phase A: 0.1% TFA in water, Mobile phase B: MeCN] to afford 2-[[4-[1-(2,6-dioxo-3-piperidyl)-3-methyl-2-oxo-benzimidazol-5-yl]-1-piperidyl]methyl]cyclopropanecarboxylic acid (3, 210 mg, 349.12 μmol, 80% yield, TFA salt) as an off-white solid.

LCMS (ES+): m/z 441.2 [M+H]⁺

### 2-(1-(1-(2,6-bis(benzyloxy)pyridin-3-yl)-3-ethyl-2-oxo-2,3-dihydro-1H-benzo[d]imidazol-5-yl)piperidin-4-yl)acetic acid (9)

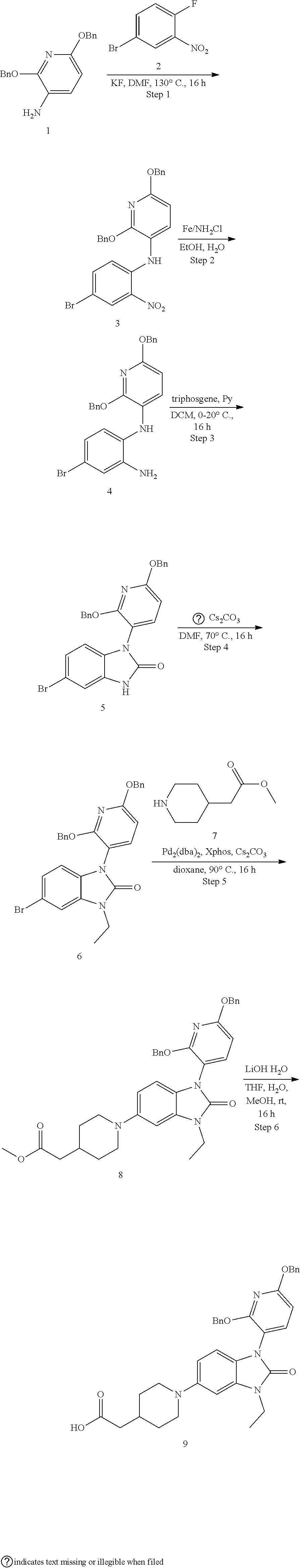

### Step 1: 2,6-bis(benzyloxy)-N-(4-bromo-2-nitrophenyl)pyridin-3-amine (3)

To a mixture of 2,6-bis(benzyloxy)pyridin-3-amine (1, 13.5 g, 44.07 mmol, 1 eq) and 4-bromo-1-fluoro-2-nitrobenzene (2, 11.63 g, 52.88 mmol, 6.50 mL, 1.2 eq) in DMF (150 mL) was added KF (3.07 g, 52.88 mmol, 1.2 eq). The mixture was stirred at 130° C. for 16 h. The residue was diluted with H₂O (1 L) and extracted with ethyl acetate (300 mL×2). The organic phase was combined and washed with brine (300 mL×2), dried over anhydrous Na₂SO₄, filtered and the filtrate concentrated under reduced pressure. The residue was purified by column chromatography (SiO₂, petroleum ether:ethyl acetate=100:0 to 0:1), then triturated with petroleum ether:ethyl acetate (100 mL, 10:1) to afford 2,6-bis(benzyloxy)-N-(4-bromo-2-nitrophenyl)pyridin-3-amine (3, 14 g, 27.65 mmol, 63% yield) as a yellow solid.

LCMS (ESI): m/z 505.8 [M+H]⁺

### Step 2: N¹-(2,6-bis(benzyloxy)pyridin-3-yl)-4-bromobenzene-1,2-diamine (4)

To a solution of 2,6-bis(benzyloxy)-N-(4-bromo-2-nitrophenyl)pyridin-3-amine (3, 14 g, 27.65 mmol, 1 eq) in EtOH (140 mL) and H₂O (140 mL) were added Fe (7.72 g, 138.25 mmol, 5 eq) and NH₄Cl (7.39 g, 138.25 mmol, 5 eq). The mixture was stirred at 70° C. for 2 h. The reaction mixture was filtered and washed with ethyl acetate (100 mL×6). The filtrate was diluted with H₂O (500 mL) and extracted with ethyl acetate (300 mL×3). The organic phases were combined and washed with brine (300 mL×3), dried over anhydrous Na₂SO₄, filtered and the filtrate concentrated under reduced pressure. The residue was purified by column chromatography (SiO₂, petroleum ether:ethyl acetate=100:0 to 0:1) to afford N¹-(2,6-bis(benzyloxy)pyridin-3-yl)-4-bromobenzene-1,2-diamine (4, 12 g, 25.19 mmol, 91% yield) as a dark solid.

LCMS (ESI): m/z 475.8 [M+H]⁺

### Step 3: 1-(2,6-bis(benzyloxy)pyridin-3-yl)-5-bromo-1H-benzo[d]imidazol-2 (3H)-one (5)

To a mixture of N¹-(2,6-bis(benzyloxy)pyridin-3-yl)-4-bromobenzene-1,2-diamine (4, 12 g, 25.19 mmol, 1 eq) and Py (19.93 g, 251.91 mmol, 20.37 mL, 10 eq) in DCM (60 mL) was added dropwise a solution of bis(trichloromethyl) carbonate (14.95 g, 50.38 mmol, 2 eq) in DCM (60 mL) at 0-10° C. The mixture was stirred at 20° C. for 16 h. The mixture was poured into ice-H₂O (300 mL) and extracted with DCM (150 mL×2). The organic phases were combined and washed with brine (150 mL×2), dried over anhydrous Na₂SO₄, filtered and the filtrate concentrated under reduced pressure. The residue was purified by column chromatography (SiO₂, petroleum ether:ethyl acetate=20:1 to 0:1) to afford 1-(2,6-bis(benzyloxy)pyridin-3-yl)-5-bromo-1H-benzo[d]imidazol-2 (3H)-one (5, 8.7 g, 16.45 mmol, 65% yield) as a off-white solid.

LCMS (ESI): m/z 501.9 [M+H]⁺

### Step 4: 1-(2,6-bis(benzyloxy)pyridin-3-yl)-5-bromo-3-ethyl-1H-benzo[d]imidazol-2 (3H)-one (6)

To a solution of 1-(2,6-bis(benzyloxy)pyridin-3-yl)-5-bromo-1H-benzo[d]imidazol-2 (3H)-one (5, 2 g, 3.98 mmol, 1 eq) in DMF (20 mL) was added Cs₂CO₃ (3.24 g, 9.95 mmol, 2.5 eq) and iodoethane (1.86 g, 11.94 mmol, 960.20 μL, 3 eq). The mixture was stirred at 60° C. for 16 h. The residue was diluted with H₂O (100 mL) and extracted with ethyl acetate (100 mL×2). The combined organic layers were washed with brine (100 mL×3), dried over Na₂SO₄, filtered and concentrated under reduced pressure. The residue was purified by column chromatography (SiO₂, petroleum ether:ethyl acetate=1:0 to 1:1) to afford 1-(2,6-bis(benzyloxy)pyridin-3-yl)-5-bromo-3-ethyl-1H-benzo[d]imidazol-2 (3H)-one (6, 2 g, 3.77 mmol, 95% yield) as a white solid.

LCMS (ESI): m/z 531.0 [M+H]⁺

### Step 5: methyl 2-(1-(1-(2,6-bis(benzyloxy)pyridin-3-yl)-3-ethyl-2-oxo-2,3-dihydro-1H-benzo[d]imidazol-5-yl)piperidin-4-yl)acetate (8)

To a solution of 1-(2,6-bis(benzyloxy)pyridin-3-yl)-5-bromo-3-ethyl-1H-benzo[d]imidazol-2 (3H)-one (6, 750 mg, 1.41 mmol, 1 eq), methyl 2-(4-piperidyl)acetate (7, 333.44 mg, 2.12 mmol, 1.5 eq) in dioxane (2 mL) were added Cs₂CO₃ (921.41 mg, 2.83 mmol, 2 eq) and XPhos (67.41 mg, 141.40 μmol, 0.1 eq), Pd₂(dba)₃ (64.74 mg, 70.70 μmol, 0.05 eq). The mixture was stirred at 90° C. for 16 h under N₂. The residue was diluted with H₂O (20 mL) and extracted with ethyl acetate (10 mL×2). The combined organic layers were washed with brine (20 mL×3), dried over Na₂SO₄, filtered and concentrated under reduced pressure. The residue was purified by column chromatography (SiO₂, petroleum ether:ethyl acetate=100:1 to 1:1) to afford methyl 2-(1-(1-(2,6-bis(benzyloxy)pyridin-3-yl)-3-ethyl-2-oxo-2,3-dihydro-1H-benzo[d]imidazol-5-yl)piperidin-4-yl)acetate (8, 480 mg, 791.15 μmol, 56% yield) as a white solid.

LCMS (ESI): m/z 607.2 [M+H]⁺

### Step 6: 2-(1-(1-(2,6-bis(benzyloxy)pyridin-3-yl)-3-ethyl-2-oxo-2,3-dihydro-1H-benzo[d]imidazol-5-yl)piperidin-4-yl)acetic acid (9)

To a solution of methyl 2-(1-(1-(2,6-bis(benzyloxy)pyridin-3-yl)-3-ethyl-2-oxo-2,3-dihydro-1H-benzo[d]imidazol-5-yl)piperidin-4-yl)acetate (8, 480 mg, 791.15 μmol, 1 eq) in MeOH (9 mL), H₂O (9 mL), THF (9 mL) was added LiOH·H₂O (332.00 mg, 7.91 mmol, 10 eq). The mixture was stirred at 50° C. for 16 h. The reaction mixture was adjusted to pH˜7 by 1 N HCl. Then the mixture was extracted with ethyl acetate (10 ml×2). The combined organic layers were washed with brine (20 mL×3), dried over Na₂SO₄, filtered and concentrated under reduced pressure. The residue was purified by column chromatography (SiO₂, petroleum ether:ethyl acetate=100:1 to 1:1) to afford 2-(1-(1-(2,6-bis(benzyloxy)pyridin-3-yl)-3-ethyl-2-oxo-2,3-dihydro-1H-benzo[d]imidazol-5-yl)piperidin-4-yl)acetic acid (9, 300 mg, 506.17 μmol, 64% yield) as a white solid.

LCMS (ESI): m/z 593.4 [M+H]⁺

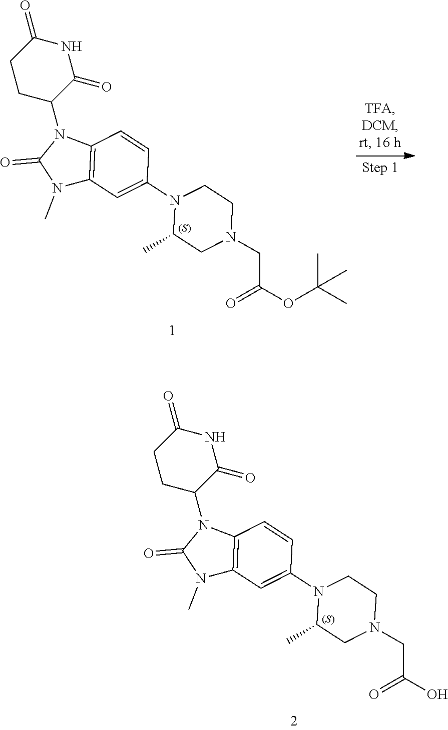

### Step 1: 2-[(3S)-4-[1-(2,6-dioxo-3-piperidyl)-3-methyl-2-oxo-benzimidazol-5-yl]-3-methyl-piperazin-1-yl]acetic acid (2)

Into a 50 mL single-neck round-bottom flask containing a well-stirred solution of tert-butyl 2-[(3S)-4-[1-(2,6-dioxo-3-piperidyl)-3-methyl-2-oxo-benzimidazol-5-yl]-3-methyl-piperazin-1-yl]acetate (1, 160 mg, 325.74 μmol) in anhydrous DCM (2.50 mL) was added TFA (1.52 g, 13.29 mmol) at 0° C. The resulting solution was stirred at ambient temperature. After 16 h the volatiles were removed under reduced pressure and the residue was triturated with pet ether (5 mL), filtered and dried to afford 2-[(3S)-4-[1-(2,6-dioxo-3-piperidyl)-3-methyl-2-oxo-benzimidazol-5-yl]-3-methyl-piperazin-1-yl]acetic acid (2, 160 mg, 296.15 μmol, 91% yield, TFA salt) as an off-white solid.

LCMS (ES+): m/z 416.2 [M+H]⁺

### 2-(5-(1-(2,6-dioxopiperidin-3-yl)-3-methyl-2-oxo-2,3-dihydro-1H-benzo[d]imidazol-5-yl)-1H-indazol-1-yl)acetic acid (6)

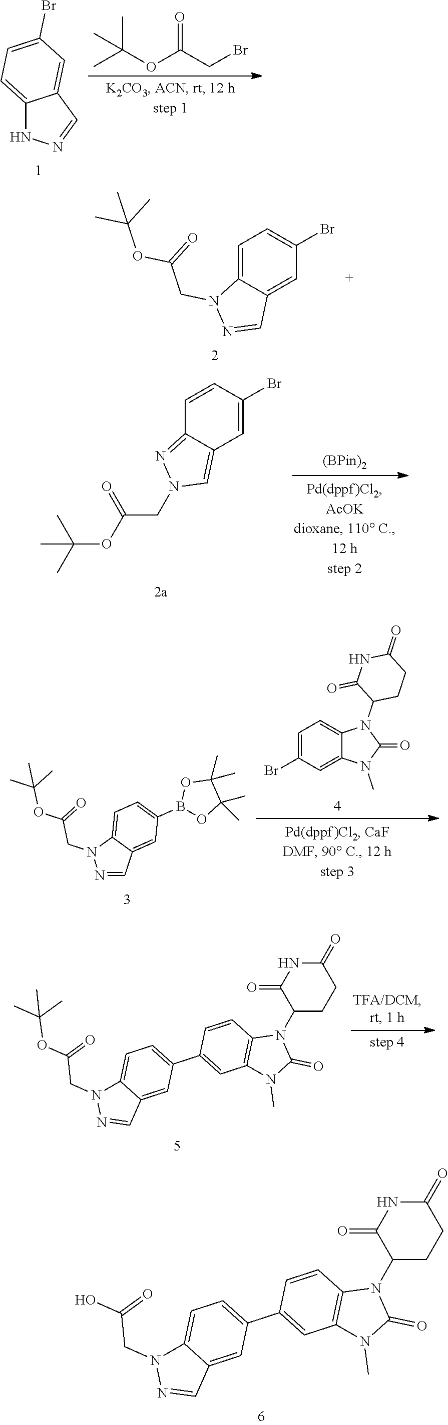

### Step 1: tert-butyl 2-(5-bromo-1H-indazol-1-yl)acetate (2) and tert-butyl 2-(5-bromo-2H-indazol-2-yl)acetate (2a)

To a solution of 5-bromo-1H-indazole (1, 6 g, 30.45 mmol, 14 eq in MeCN (50 mL) were added tert-butyl 2-bromoacetate (7.13 g, 36.54 mmol, 1.2 eq and K₂CO₃ (10.1 g, 73.08 mmol, 2.4 eq). The mixture was stirred at 25° C. for 12 h. The mixture was diluted with EtOAc (80 mL) and water (80 mL). The organic layer was washed with brine (100 mL), dried over Na₂SO₄, filtered and concentrated in vacuo. The residue was purified by flash silica gel chromatography (ISCO©, 120 g Silica Flash Column, eluent of 20˜30% ethyl acetate/petroleum ether gradient @100 mL/min) to give tert-butyl 2-(5-bromo-1H-indazol-1-yl)acetate (2, 7.5 g, 22.9 mmol, 75% yield) as white solid.

LCMS (ESI): m/z 257.0/255.0 [M−tBu+H]⁺

¹H NMR (400 MHz, CDCl3) δ=7.99 (d, J=0.8 Hz, 1H), 7.89 (d, J=1.6 Hz, 1H), 7.48 (dd, J=2.0, 9.2 Hz, 1H), 7.23 (d, J=9.2 Hz, 1H), 5.04 (s, 2H), 1.44 (s, 9H).

And tert-butyl 2-(5-bromo-2H-indazol-2-yl)acetate (2a, 3.5 g, 10.12 mmol, 33% yield) as white solid.

LCMS (ESI): m/z 257.0/255.0 [M−tBu+H]⁺

¹H NMR (400 MHz, CDCl3) δ=7.95 (d, J=0.8 Hz, 1H), 7.83 (dd, J=0.7, 1.6 Hz, 1H), 7.59 (td, J=0.8, 9.2 Hz, 1H), 7.34 (dd, J=1.6, 9.2 Hz, 1H), 5.09 (s, 2H), 1.49 (s, 9H).

### Step 2: tert-butyl 2-(5-(4,4,5,5-tetramethyl-1,3,2-dioxaborolan-2-yl)-1H-indazol-1-yl)acetate (3)

To a solution of tert-butyl 2-(5-bromo-1H-indazol-1-yl)acetate (2, 0.5 g, 1.61 mmol, 1 eq) in dioxane (5 mL) were added Pd(dppf)Cl₂ (58 mg, 79.27 μmol, 0.05 eg), 4,4,4′,4′,5,5,5′,5′-octamethyl-2,2′-bi(1,3,2-dioxaborolane) (816 mg, 3.21 mmol, 2 eq) and KOAc (473 mg, 4.82 mmol, 3 eq). The mixture was stirred at 110° C. for 12 h under N₂. After the reaction mixture was cooled to room temperature, EtOAc (40 mL) and water (40 mL) were added and layers were separated. The aqueous phase was extracted with EtOAc (30 mL×2). Combined extracts were washed with brine (30 mL), dried over MgSO₄, filtered, and concentrated under vacuum. The residue was purified by flash silica gel chromatography (ISCO®, 10 g Silica Flash Column, eluent of 24% ethyl acetate/petroleum ether gradient @100 mL/min) to give tert-butyl 2-(5-(4,4,5,5-tetramethyl-1,3,2-dioxaborolan-2-yl)-1H-indazol-1-yl)acetate (3, 500 mg, 1.23 mmol, 76% yield) as yellow oil.

LCMS (ESI): m/z 303.1 [M−tBu+H]⁺

### Step 3: tert-butyl 2-(5-(1-(2,6-dioxopiperidin-3-yl)-3-methyl-2-oxo-2,3-dihydro-1H-benzo[d]imidazol-5-yl)-1H-indazol-1-yl)acetate (5)

To a solution of tert-butyl 2-(5-(4,4,5,5-tetramethyl-1,3,2-dioxaborolan-2-yl)-1H-indazol-1-yl)acetate (3, 466 mg, 1.30 mmol, 1 eq) in DMF (3 mL) were added CsF (360 mg, 2.37 mmol, 2 eq) 3-(5-bromo-3-methyl-2-oxo-2,3-dihydro-1H-benzo[d]imidazol-1-yl)piperidine-2,6-dione (4, 400 mg, 1.18 mmol, 1.1 eq) and Pd(dppf)Cl₂ (86.6 mg, 118 μmol, 0.1 eq). The mixture was stirred at 90° C. under N₂ for 12 h. The reaction was filtered, the filtrate was purified by reverse phase column (0.1% FA in water/acetonitrile) to give tert-butyl 2-(5-(1-(2,6-dioxopiperidin-3-yl)-3-methyl-2-oxo-2,3-dihydro-1H-benzo[d]imidazol-5-yl)-1H-indazol-1-yl)acetate (5, 250 mg, 393 μmol, 33% yield) as a yellow solid.

LCMS (ES+): m/z 490.4 [M+H]⁺

### Step 4: 2-(5-(1-(2,6-dioxopiperidin-3-yl)-3-methyl-2-oxo-2,3-dihydro-1H-benzo[d]imidazol-5-yl)-1H-indazol-1-yl)acetic acid (6)

A solution of tert-butyl 2-(5-(1-(2,6-dioxopiperidin-3-yl)-3-methyl-2-oxo-2,3-dihydro-1H-benzo[d]imidazol-5-yl)-1H-indazol-1-yl)acetate (5, 250 mg, 510 μmol, 1 eq) in TFA (1 mL) and DCM (5 mL) was stirred at 25° C. for 1 h. The mixture was concentrated to give a residue. The residue was purified by reverse phase column chromatography (0.1% FA in water/acetonitrile) to give 2-(5-(1-(2,6-dioxopiperidin-3-yl)-3-methyl-2-oxo-2,3-dihydro-1H-benzo[d]imidazol-5-yl)-1H-indazol-1-yl)acetic acid (6, 110 mg, 249 μmol, 49% yield) as a yellow solid.

LCMS (ESI+): m/z 434.2 [M+H]⁺

¹H NMR (400 MHz, DMSO-d₆) δ=13.24-12.93 (m, 1H), 11.13 (s, 1H), 8.14 (s, 1H), 8.05 (s, 1H), 7.78-7.70 (m, 2H), 7.56 (d, J=1.6 Hz, 1H), 7.40 (dd, J=1.6, 8.4 Hz, 1H), 7.21 (d, J=8.4 Hz, 1H), 5.42 (dd, J=5.6, 13.2 Hz, 1H), 5.30 (s, 2H), 3.43 (s, 3H), 3.03-2.87 (m, 1H), 2.86-2.73 (m, 1H), 2.72-2.61 (m, 2H), 2.13-2.02 (m, 1H)

### 3-(4-(3-(2,6-dioxopiperidin-3-yl)-1-methyl-1H-indazol-6-yl)piperidin-1-yl)cyclobutanecarboxylic acid (5)

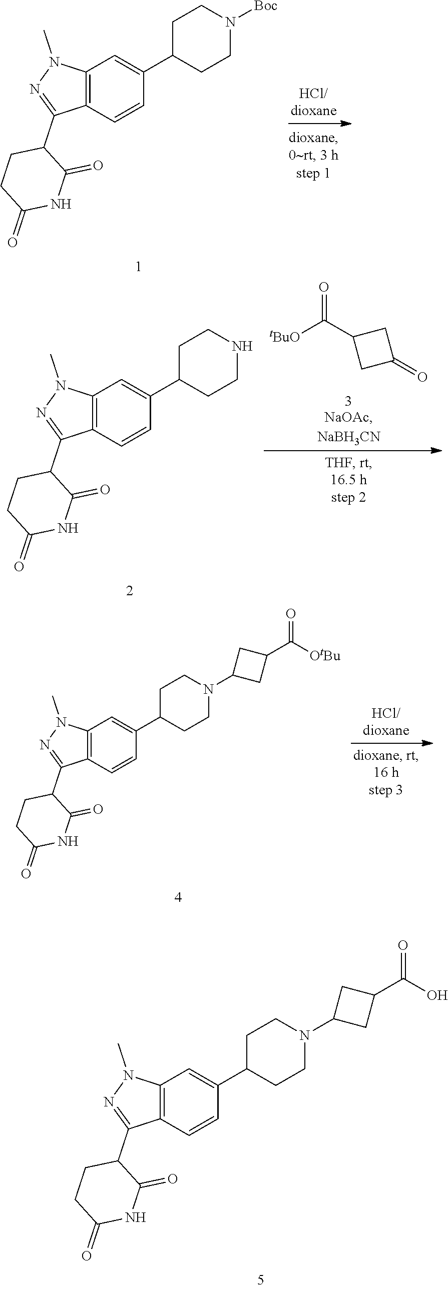 [SMILES: Cn1nc(C2CCC(=O)NC2=O)c2ccc(C3CCN(C(=O)OC(C)(C)C)CC3)cc21 ; Cn1nc(C2CCC(=O)NC2=O)c2ccc(C3CCNCC3)cc21 ; CC(C)(C)OC(=O)C1CC(=O)C1 ; Cn1nc(C2CCC(=O)NC2=O)c2ccc(C3CCN(C4CC(C(=O)OC(C)(C)C)C4)CC3)cc21 ; Cn1nc(C2CCC(=O)NC2=O)c2ccc(C3CCN(C4CC(C(=O)O)C4)CC3)cc21]

### Step 1: 3-(1-methyl-6-(piperidin-4-yl)-1H-indazol-3-yl)piperidine-2,6-dione (2)

To a solution of tert-butyl 4-(3-(2,6-dioxopiperidin-3-yl)-1-methyl-1H-indazol-6-yl)piperidine-1-carboxylate (1, 600 mg, 1.41 mmol, 1 eq) in dioxane (2 mL) was added HCl/dioxane (4 M, 8 mL) at 0° C. The mixture was stirred at 20° C. for 3 h. The reaction mixture was concentrated under reduced pressure to afford 3-(1-methyl-6-(piperidin-4-yl)-1H-indazol-3-yl)piperidine-2,6-dione (2, 600 mg, 1.41 mmol, 99% yield, HCl salt) as a white solid. The crude product was used in the next step without further purification.

LCMS (ESI): m/z 327.3 [M+H]⁺

### Step 2: tert-butyl 3-(4-(3-(2,6-dioxopiperidin-3-yl)-1-methyl-1H-indazol-6-yl)piperidin-1-yl)cyclobutanecarboxylate (4)

To a solution of 3-(1-methyl-6-(piperidin-4-yl)-1H-indazol-3-yl)piperidine-2,6-dione (2, 600 mg, 1.65 mmol, HCl salt, 1 eq) and tert-butyl 3-oxocyclobutanecarboxylate (3, 281.44 mg, 1.65 mmol, 1 eq) in THF (20 mL) was added NaOAc (271.30 mg, 3.31 mmol, 2 eq). The mixture was stirred at 20° C. for 0.5 h, then added NaBH₃CN (519.57 mg, 8.27 mmol, 5 eq) and stirred at 20° C. for 16 h. The reaction mixture was concentrated under reduced pressure to give a residue. The residue was purified by flash silica gel chromatography (60 mL/min, Eluent of 0-100% ethyl acetate/petroleum ether gradient, Column: ISCO®; 10 g Sepa Flash® Silica Flash column) to afford tert-butyl 3-(4-(3-(2,6-dioxopiperidin-3-yl)-1-methyl-1H-indazol-6-yl)piperidin-1-yl)cyclobutanecarboxylate (4, 500 mg, 832.30 μmol, 50% yield) as a white solid.

LCMS (ESI): m/z 481.3 [M+H]⁺

### Step 3: 3-(4-(3-(2,6-dioxopiperidin-3-yl)-1-methyl-1H-indazol-6-yl)piperidin-1-yl)cyclobutanecarboxylic acid (5)

To a solution of tert-butyl 3-(4-(3-(2,6-dioxopiperidin-3-yl)-1-methyl-1H-indazol-6-yl)piperidin-1-yl)cyclobutanecarboxylate (4, 400 mg, 832.30 μmol, 1 eq) in dioxane (1 mL) was added HCl/dioxane (832.30 μmol, 10 mL) at 0° C. and stirred at 20° C. for 3 h. The reaction mixture was concentrated under reduced pressure to afford 3-(4-(3-(2,6-dioxopiperidin-3-yl)-1-methyl-1H-indazol-6-yl)piperidin-1-yl)cyclobutanecarboxylic acid (5, 400 mg, 728.93 μmol, 88% yield, HCl salt) as a white solid. The crude product was used in the next step without further purification.

LCMS (ESI): m/z 425.2 [M+H]⁺

### 2-[4-[4-chloro-1-(2,6-dioxo-3-piperidyl)-3-methyl-2-oxo-benzimidazol-5-yl]-1-piperidyl]acetic acid (3)

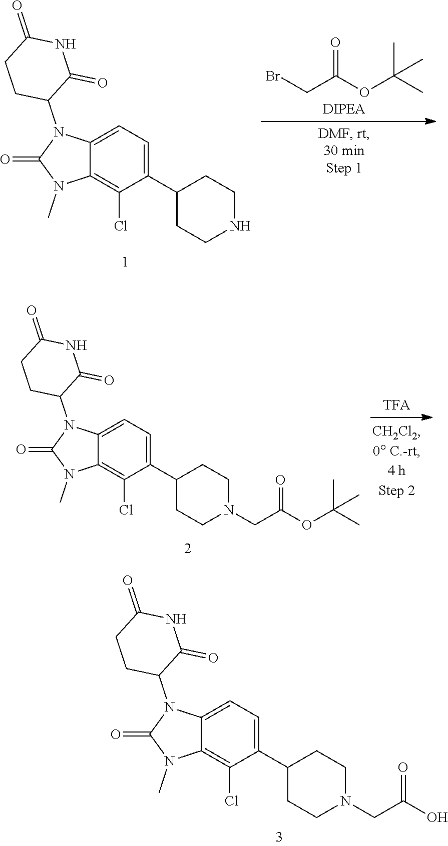

### Step 1: tert-butyl 2-[4-[4-chloro-1-(2,6-dioxo-3-piperidyl)-3-methyl-2-oxo-benzimidazol-5-yl]-1-piperidyl]acetate (2)

Into a 20 mL screw-capped vial containing a well-stirred solution of 3-[4-chloro-3-methyl-2-oxo-5-(4-piperidyl)benzimidazol-1-yl]piperidine-2,6-dione (1, 200 mg, 407.45 μmol, TFA salt) in DMF (2.0 mL) under nitrogen atmosphere at 0° C. was added DIPEA (210.63 mg, 1.63 mmol, 283.87 μL) and tert-butyl 2-bromoacetate (55.63 mg, 285.21 μmol, 41.83 μL) and the reaction mixture was stirred at ambient temperature for 30 min. The reaction mixture was concentrated under vacuum and diluted with ice cold water to precipitate a solid that was filtered, washed with water and dried under vacuum to get tert-butyl 2-[4-[4-chloro-1-(2,6-dioxo-3-piperidyl)-3-methyl-2-oxo-benzimidazol-5-yl]-1-piperidyl]acetate (2, 100 mg, 180.94 μmol, 44% yield) as an off white solid.

LCMS (ES+): m/z 491.2 [M+H]⁺

### Step 2: 2-[4-[4-chloro-1-(2,6-dioxo-3-piperidyl)-3-methyl-2-oxo-benzimidazol-5-yl]-1-piperidyl]acetic acid (3)

Into a 25 mL single-neck round-bottom flask containing a well-stirred solution of tert-butyl 2-[4-[4-chloro-1-(2,6-dioxo-3-piperidyl)-3-methyl-2-oxo-benzimidazol-5-yl]-1-piperidyl]acetate (2, 100 mg, 179.23 μmol) in DCM (2 mL) was added TFA (408.74 mg, 3.58 mmol, 276.17 μL) at 0° C. The reaction mixture was stirred at ambient temperature for 4 h. The reaction mixture was evaporated and the residue was triturated with diethyl ether to get 2-[4-[4-chloro-1-(2,6-dioxo-3-piperidyl)-3-methyl-2-oxo-benzimidazol-5-yl]-1-piperidyl]acetic acid (3, 110 mg, 174.35 μmol, 97% yield, TFA salt) as an off-white solid.

LCMS (ES+): m/z 435.2 [M+H]⁺

### 2-[4-[1-(2,6-dioxo-3-piperidyl)-3-methyl-2-oxo-benzimidazol-5-yl]-2-oxo-1-pyridyl]acetic acid (6)

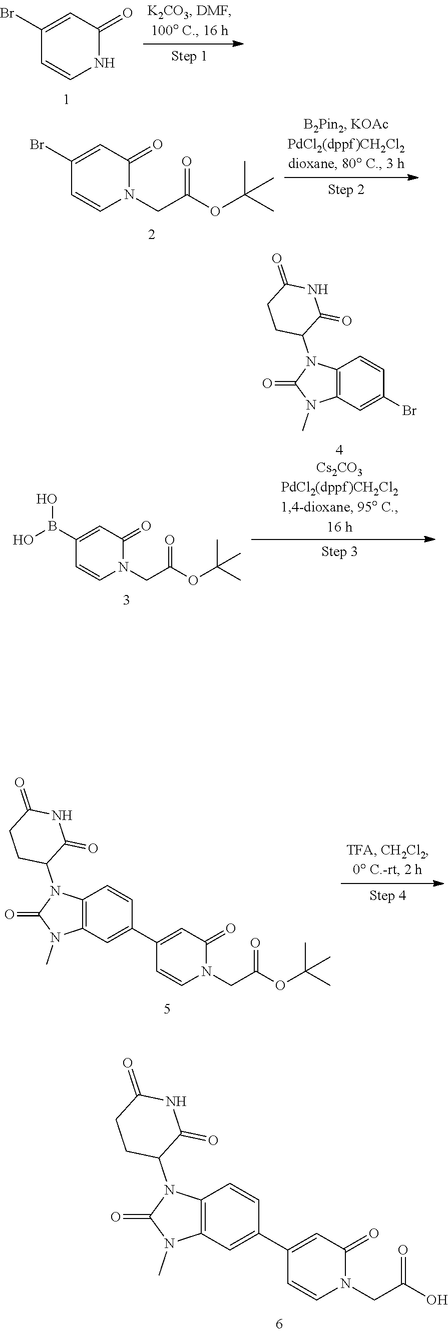 [SMILES: O=c1cc(Br)cc[nH]1 ; CC(C)(C)OC(=O)Cn1ccc(Br)cc1=O ; Cn1c(=O)n(C2CCC(=O)NC2=O)c2ccc(Br)cc21 ; CC(C)(C)OC(=O)Cn1ccc(B(O)O)cc1=O ; Cn1c(=O)n(C2CCC(=O)NC2=O)c2ccc(-c3ccn(CC(=O)OC(C)(C)C)c(=O)c3)cc21 ; Cn1c(=O)n(C2CCC(=O)NC2=O)c2ccc(-c3ccn(CC(=O)O)c(=O)c3)cc21]

### Step 1: tert-butyl 2-(4-bromo-2-oxo-1-pyridyl)acetate (2)

Into a 100 mL round bottom flask contain well-stirred solution of 4-bromopyridin-2 (1H)-one (1, 0.85 g, 4.89 mmol) in DMF (8 mL) was added tert-butyl 2-bromoacetate (1.14 g, 5.86 mmol) and potassium carbonate (1.01 g, 7.33 mmol) at room temperature. The reaction mixture was stirred at 100° C. After 16 h, the reaction mixture was quenched with water (40 mL) and extracted with ethyl acetate (70 mL×3). The combined organic layer was washed with water (150 mL) followed by brine (100 mL) and dried over sodium sulfate. The solvent was removed under reduced pressure and the crude product was purified by flash silica gel column chromatography (35-40% of EtOAc in pet ether) to obtain tert-butyl 2-(4-bromo-2-oxo-1-pyridyl)acetate (2, 1.26 g, 4.26 mmol, 87% yield) as a colorless solid.

LCMS (ES+): m/z 233.0 [M−tBu+H]⁺

### Step 2: (1-(2-(tert-butoxy)-2-oxoethyl)-2-oxo-1,2-dihydropyridin-4-yl)boronic acid (3)

Into a 100 mL pressure tube containing a well-stirred solution of tert-butyl 2-(4-bromo-2-oxo-1-pyridyl)acetate (2, 1.0 g, 3.37 mmol) and bis(pinacolato) diboron (2.56 g, 10.10 mmol) in 1,4-dioxane (40 mL) was added potassium acetate (495.58 mg, 5.05 mmol) at room temperature. The mixture was degassed by bubbling nitrogen gas for 10 min. Then 1,1′-bis(diphenylphosphino)ferrocene]dichloropalladium(II) complex with dichloromethane (82.48 mg, 100.99 μmol) was added. The reaction mixture was heated at 80° C. The reaction mixture was filtered through Celite and the filtrate was diluted with water (35 mL) and extracted with 10% MeOH in EtOAc (2×80 mL). The combined organic layer was washed with brine solution (50 mL), dried over anhydrous Na₂SO₄ and filtered. The solvent was removed under reduced pressure to obtain (1-(2-(tert-butoxy)-2-oxoethyl)-2-oxo-1,2-dihydropyridin-4-yl)boronic acid (3, 1.02 g, 64% yield) as a yellow solid.

LCMS (ES+): m/z 254.2 [M+H]⁺

### Step 3: tert-butyl 2-[4-[1-(2,6-dioxo-3-piperidyl)-3-methyl-2-oxo-benzimidazol-5-yl]-2-oxo-1-pyridyl]acetate (5)

Into a 100 mL pressure tube containing a well-stirred solution of 3-(5-bromo-3-methyl-2-oxo-benzimidazol-1-yl)piperidine-2,6-dione (4, 0.5 g, 1.40 mmol) and [1-(2-tert-butoxy-2-oxo-ethyl)-2-oxo-4-pyridyl]boronic acid (3, 807.88 mg, 2.11 mmol) in 1,4-dioxane (30 mL) was added cesium carbonate (915.34 mg, 2.81 mmol) at room temperature. The suspension was degassed by purging nitrogen gas for 10 min. Then PdCl₂(dppf)·DCM (344.12 mg, 421.40 μmol) was added and the reaction mixture was stirred at 95° C. After 16 h, the reaction mixture was filtered through Celite. The filtrate was concentrated under reduced pressure to get the crude compound, which was purified by flash silica gel column chromatography (5% MeOH in DCM) to obtain tert-butyl 2-[4-[1-(2,6-dioxo-3-piperidyl)-3-methyl-2-oxo-benzimidazol-5-yl]-2-oxo-1-pyridyl]acetate (5, 0.51 g, 929.29 μmol, 66% yield) as a light brown solid.

LCMS (ES+): m/z 467.2 [M+H]⁺

### Step 4: 2-[4-[1-(2,6-dioxo-3-piperidyl)-3-methyl-2-oxo-benzimidazol-5-yl]-2-oxo-1-pyridyl]acetic acid (6)

Into a 50 mL round bottom flask containing a well-stirred solution tert-butyl 2-[4-[1-(2,6-dioxo-3-piperidyl)-3-methyl-2-oxo-benzimidazol-5-yl]-2-oxo-1-pyridyl]acetate (5, 0.51 g, 929.29 μmol) in DCM (7 mL) was added trifluoroacetic acid (2.22 g, 19.47 mmol) at 0° C. and stirred at room temperature. After 4 h the solvent was removed under reduced pressure, the residue was triturated with MTBE (20 mL), filtered and dried to afford 2-[4-[1-(2,6-dioxo-3-piperidyl)-3-methyl-2-oxo-benzimidazol-5-yl]-2-oxo-1-pyridyl]acetic acid (6, 0.43 g, 884.67 μmol, 95% yield, TFA salt) as a light brown solid.

LCMS (ES+): m/z 410.8 [M+H]⁺

### 3-[3-methyl-2-oxo-5-(4-piperidylmethyl)benzimidazol-1-yl]piperidine-2,6-dione (4)

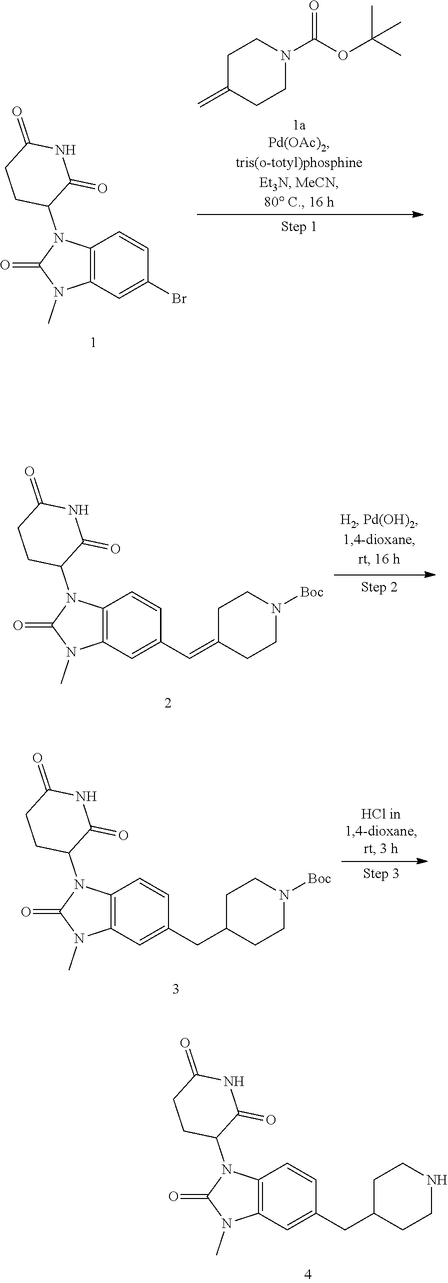 [SMILES: C=C1CCN(C(=O)OC(C)(C)C)CC1 ; Cn1c(=O)n(C2CCC(=O)NC2=O)c2ccc(Br)cc21 ; Cn1c(=O)n(C2CCC(=O)NC2=O)c2ccc(C=C3CCN(C(=O)OC(C)(C)C)CC3)cc21 ; Cn1c(=O)n(C2CCC(=O)NC2=O)c2ccc(CC3CCN(C(=O)OC(C)(C)C)CC3)cc21 ; Cn1c(=O)n(C2CCC(=O)NC2=O)c2ccc(CC3CCNCC3)cc21]

### Step 1: tert-butyl 4-[[1-(2,6-dioxo-3-piperidyl)-3-methyl-2-oxo-benzimidazol-5-yl]methylene]piperidine-1-carboxylate (2)

Into a 25 mL pressure tube containing a well-stirred solution of 3-(5-bromo-3-methyl-2-oxo-benzimidazol-1-yl)piperidine-2,6-dione (1, 1 g, 2.96 mmol) and tert-butyl 4-methylenepiperidine-1-carboxylate (1a, 700.06 mg, 3.55 mmol) in anhydrous acetonitrile (10 mL) was added triethylamine (2.90 g, 28.70 mmol, 4 mL) at room temperature under nitrogen atmosphere. The mixture was degassed by bubbling nitrogen gas for 5 min. Then tris(o-tolyl)phosphine (90.01 mg, 295.72 μmol) and palladium (II) acetate (99.59 mg, 443.58 μmol) were added and degassed for additional 5 min. The reaction mixture was heated to 90° C. After 16 h the reaction mixture was cooled and filtered through Celite and washed with ethyl acetate (100 mL). The filtrate was concentrated to dryness under reduced pressure and the residue was purified by reverse phase column chromatography (Purification method: Siliasep premium C18, um 120 g column; Mobile phase A: 0.1% TFA in water; Mobile phase B: Acetonitrile) to afford tert-butyl 4-[[1-(2,6-dioxo-3-piperidyl)-3-methyl-2-oxo-benzimidazol-5-yl]methylene]piperidine-1-carboxylate (2, 600 mg, 1.14 mmol, 38% yield) as a yellow solid.

LCMS (ES+): m/z 399.2 [M−tBu+H]⁺

### Step 2: tert-butyl4-[[1-(2,6-dioxo-3-piperidyl)-3-methyl-2-oxo-benzimidazol-5-yl]methyl]piperidine-1-carboxylate (3)

Into a 100 mL single neck round bottom flask containing a well-stirred solution of tert-butyl 4-[[1-(2,6-dioxo-3-piperidyl)-3-methyl-2-oxo-benzimidazol-5-yl]methylene]piperidine-1-carboxylate (2, 600 mg, 1.10 mmol) in 1,4-dioxane (10 mL) was added palladium hydroxide on carbon, 20 wt. % dry basis (600.09 mg, 854.62 μmol, 20% purity). The suspension was stirred under hydrogen atmosphere at room temperature. After 16 h, the reaction mixture was filtered through Celite and washed with 1,4-dioxane (100 mL). The filtrate was concentrated under reduced pressure to obtain crude tert-butyl 4-[[1-(2,6-dioxo-3-piperidyl)-3-methyl-2-oxo-benzimidazol-5-yl]methyl]piperidine-1-carboxylate (3, 540 mg, 1.08 mmol, 98% yield) as a pale yellow gummy liquid.

LCMS (ES+): m/z 357.2 [M−Boc+H]⁺

### Step 3: 3-[3-methyl-2-oxo-5-(4-piperidylmethyl)benzimidazol-1-yl]piperidine-2,6-dione (4)

Into a 100 mL single round bottom flask containing a well-stirred solution of tert-butyl 4-[[1-(2,6-dioxo-3-piperidyl)-3-methyl-2-oxo-benzimidazol-5-yl]methyl]piperidine-1-carboxylate (3, 560 mg, 1.12 mmol) in 1,4-dioxane (6 mL) was added a 4.0 M solution of hydrogen in dioxane (1.60 g, 43.88 mmol, 2 mL) at room temperature. After 3 h, the reaction mixture was concentrated to dryness under reduced pressure and washed with diethyl ether (2×10 mL) and dried to get 3-[3-methyl-2-oxo-5-(4-piperidylmethyl)benzimidazol-1-yl]piperidine-2,6-dione (4, 500 mg, 1.09 mmol, 98% yield, HCl salt) as a pale yellow solid.

LCMS: m/z 357.2 [M+H]⁺

### 2-[(3R)-4-[1-(2,6-dioxo-3-piperidyl)-3-methyl-2-oxo-benzimidazol-5-yl]-3-methyl-piperazin-1-yl]acetic acid (2)

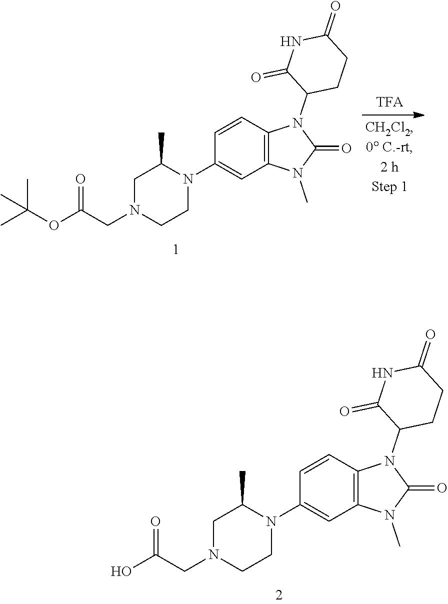 [SMILES: C[C@@H]1CN(CC(=O)OC(C)(C)C)CCN1c1ccc2c(c1)n(C)c(=O)n2C1CCC(=O)NC1=O ; C[C@@H]1CN(CC(=O)O)CCN1c1ccc2c(c1)n(C)c(=O)n2C1CCC(=O)NC1=O]

### Step 1: 2-[(3R)-4-[1-(2,6-dioxo-3-piperidyl)-3-methyl-2-oxo-benzimidazol-5-yl]-3-methyl-piperazin-1-yl]acetic acid (2)

Into a 25 mL single-neck round-bottom flask containing a well-stirred solution of tert-butyl 2-[(3R)-4-[1-(2,6-dioxo-3-piperidyl)-3-methyl-2-oxo-benzimidazol-5-yl]-3-methyl-piperazin-1-yl]acetate (1, 200 mg, 424.13 μmol) in dry DCM (5 mL) under nitrogen atmosphere was added TFA (483.61 mg, 4.24 mmol, 326.76 μL) at 0° C. The resulting reaction mixture was stirred at room temperature for 2 h. The reaction mixture was concentrated under reduced pressure to obtain the crude material that was triturated with MTBE (25 mL) to afford 2-[(3R)-4-[1-(2,6-dioxo-3-piperidyl)-3-methyl-2-oxo-benzimidazol-5-yl]-3-methyl-piperazin-1-yl]acetic acid (2, 220 mg, 394.74 μmol, 93% yield, TFA salt) as an off-white solid.

LCMS (ES+): m/z 416.0 [M+H]⁺

### 2-(1-(1-(2,6-bis(benzyloxy)pyridin-3-yl)-3-isopropyl-2-oxo-2,3-dihydro-1H-benzo[d]imidazol-5-yl)piperidin-4-yl)acetic acid (6)

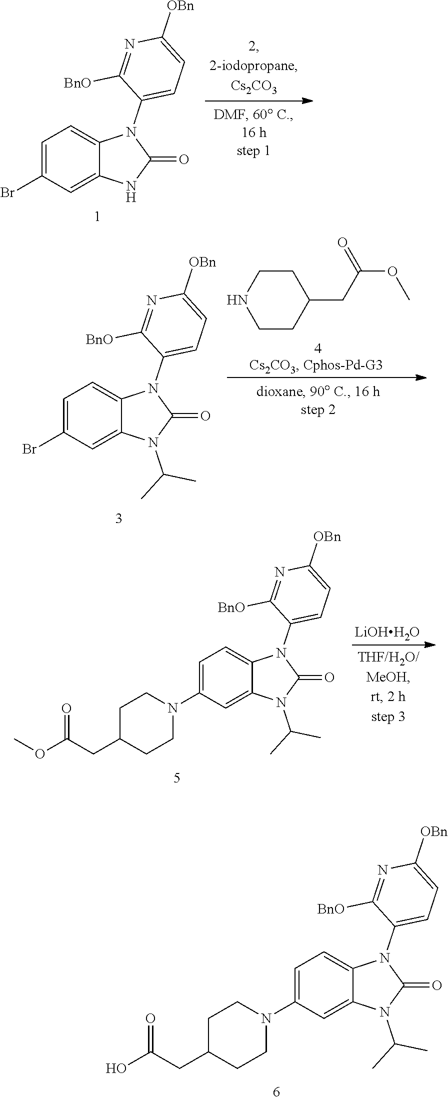

### Step 1:1-(2,6-bis(benzyloxy)pyridin-3-yl)-5-bromo-3-isopropyl-1H-benzo[d]imidazol-2 (3H)-one (3)

To a solution of 1-(2,6-bis(benzyloxy)pyridin-3-yl)-5-bromo-1H-benzo[d]imidazol-2 (3H)-one (1, 2 g, 3.98 mmol, 1 eq) in DMF (20 mL) was added Cs₂CO₃ (3.24 g, 9.95 mmol, 2.5 eq) and 2-iodopropane (2, 2.03 g, 11.94 mmol, 1.19 mL, 3 eq). The mixture was stirred at 60° C. for 16 h. The mixture was diluted with water (50 mL) and extracted with ethyl acetate (50 mL×3). The combined organic layers were washed with brine (80 mL), dried over Na₂SO₄, filtered and concentrated under reduced pressure. The residue was purified by column chromatography (SiO₂, petroleum ether/ethyl acetate=100/1 to 3/1) to afford 1-(2,6-bis(benzyloxy)pyridin-3-yl)-5-bromo-3-isopropyl-1H-benzo[d]imidazol-2 (3H)-one (3, 1.8 g, 3.31 mmol, 83% yield) as a yellow solid.

LCMS (ESI): m/z 544.1 [M+H]⁺

### Step 2: methyl 2-(1-(1-(2,6-bis(benzyloxy)pyridin-3-yl)-3-isopropyl-2-oxo-2,3-dihydro-1H-benzo[d]imidazol-5-yl)piperidin-4-yl)acetate (5)

A mixture of 1-(2,6-bis(benzyloxy)pyridin-3-yl)-5-bromo-3-isopropyl-1H-benzo[d]imidazol-2 (3H)-one (3, 800 mg, 1.47 mmol, 1 eq) and methyl 2-(piperidin-4-yl)acetate (4, 462.01 mg, 2.94 mmol, 2 eq) in dioxane (8 mL) were added Cphos-Pd-G3 (118.43 mg, 146.94 μmol, 0.1 eq) and Cs₂CO₃ (1.44 g, 4.41 mmol, 3 eq), Then the mixture was degassed and purged with N₂ 3 times, and the mixture was stirred at 90° C. for 16 h under N₂ atmosphere. The reaction was filtered and concentrated under reduced pressure to give a residue. The residue was purified by flash silica gel chromatography (60 mL/min, Eluent of 0-50% ethyl acetate/petroleum ether gradient, Column: ISCO®; 20 g Sepa Flash® Silica Flash Column) to afford 1-(2,6-bis(benzyloxy)pyridin-3-yl)-5-bromo-3-(oxetan-3-yl)-1H-benzo[d]imidazol-2 (3H)-one (5, 1 g, 1.50 mmol, 91% yield) as a yellow oil.

LCMS (ESI): m/z 621.2 [M+H]⁺

### Step 3: 2-(1-(1-(2,6-bis(benzyloxy)pyridin-3-yl)-3-isopropyl-2-oxo-2,3-dihydro-1H-benzo[d]imidazol-5-yl)piperidin-4-yl)acetic acid (6)

To a solution of 1-(2,6-bis(benzyloxy)pyridin-3-yl)-5-bromo-3-(oxetan-3-yl)-1H-benzo[d]imidazol-2 (3H)-one (5, 1 g, 1.61 mmol, 1 eq) in H₂O (8 mL), MeOH (8 mL) and THF (8 mL) was added LiOH H₂O (338.02 mg, 8.05 mmol, 5 eq). The mixture was stirred at 20° C. for 2 h. The reaction mixture was acidified to pH=5 with 1 N aqueous HCl. Then the mixture was diluted with water (20 mL) and extracted with ethyl acetate (20 mL×3). The combined organic layers were washed with brine (30 mL), dried over Na₂SO₄, filtered and concentrated under reduced pressure to afford 2-(1-(1-(2,6-bis(benzyloxy)pyridin-3-yl)-3-isopropyl-2-oxo-2,3-dihydro-1H-benzo[d]imidazol-5-yl)piperidin-4-yl)acetic acid (6, 960 mg, 1.47 mmol, 91% yield) as a yellow solid. The crude product was used in the next step without further purification.

LCMS (ESI): m/z 607.2 [M+H]⁺

### 2-[4-[1-(2,6-dioxo-3-piperidyl)-3-methyl-2-oxo-benzimidazol-5-yl]-3-fluoro-1-piperidyl]acetic acid (6)

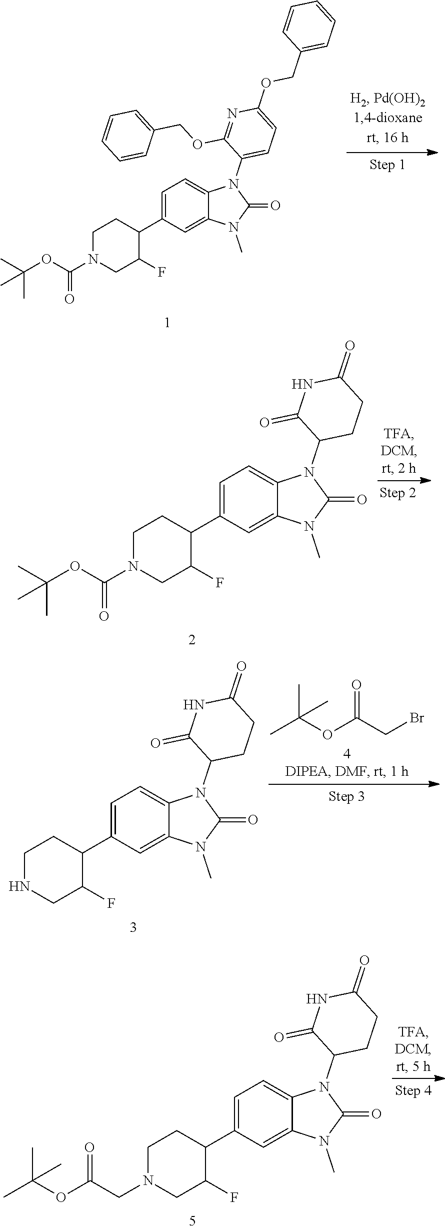

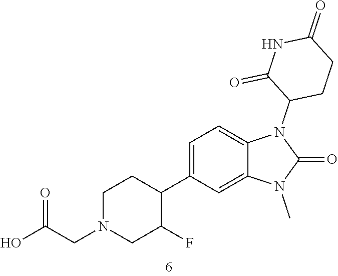 [SMILES: Cn1c(=O)n(C2CCC(=O)NC2=O)c2ccc(C3CCN(CC(=O)O)CC3F)cc21]

### Step 1: tert-butyl 4-[1-(2,6-dioxo-3-piperidyl)-3-methyl-2-oxo-benzimidazol-5-yl]-3-fluoro-piperidine-1-carboxylate (2)

Into a 100 mL single-neck round-bottom flask containing a well-stirred solution of tert-butyl 4-[1-(2,6-dibenzyloxy-3-pyridyl)-3-methyl-2-oxo-benzimidazol-5-yl]-3-fluoro-piperidine-1-carboxylate (1, 700 mg, 1.10 mmol) in 1,4-dioxane (12 mL) was added palladium hydroxide on carbon, 20 wt. % (560.00 mg, 797.52 μmol). After 16 h, the reaction mixture was filtered through Celite, washed with 1,4-dioxane (100 mL). The filtrate was concentrated under reduced pressure to get tert-butyl 4-[1-(2,6-dioxo-3-piperidyl)-3-methyl-2-oxo-benzimidazol-5-yl]-3-fluoro-piperidine-1-carboxylate (2, 470 mg, 994.41 μmol, 91% yield) as a colorless solid.

LCMS (ES+): m/z 405.2 [M-tBu+H]⁺

### Step 2: 3-[5-(3-fluoro-4-piperidyl)-3-methyl-2-oxo-benzimidazol-1-yl]piperidine-2,6-dione (3)

Into a 100 mL single-neck round-bottom flask containing a well-stirred solution of tert-butyl 4-[1-(2,6-dioxo-3-piperidyl)-3-methyl-2-oxo-benzimidazol-5-yl]-3-fluoro-piperidine-1-carboxylate (2, 470 mg, 994.41 μmol) in anhydrous DCM (10 mL) was added TFA (1.48 g, 12.98 mmol) at room temperature. The resulting solution was stirred at room temperature for 2 h. The volatiles were removed to dryness and the residue was triturated with MTBE (50 mL), filtered and dried. The crude compound was purified by reverse phase prep HPLC [Purification method: Column: XSelect C18 (150×19) mm 5 micron; Mobile phase A: 0.1% TFA in water; Mobile phase B: MeCN] to get 3-[5-(3-fluoro-4-piperidyl)-3-methyl-2-oxo-benzimidazol-1-yl]piperidine-2,6-dione (3, 420 mg, 865.13 μmol, 87% yield, TFA salt) as a colorless solid.

LCMS (ES+): m/z 361.1 [M+H]⁺

### Step 3: tert-butyl 2-[4-[1-(2,6-dioxo-3-piperidyl)-3-methyl-2-oxo-benzimidazol-5-yl]-3-fluoro-1-piperidyl]acetate (5)

Into a 25 mL single-neck round-bottom flask containing a well-stirred solution of 3-[5-(3-fluoro-4-piperidyl)-3-methyl-2-oxo-benzimidazol-1-yl]piperidine-2,6-dione (3, 200 mg, 404.72 μmol, TFA salt) in anhydrous DMF (3 mL) were added DIPEA (156.92 mg, 1.21 mmol, 211.48 μL) and tert-butyl 2-bromoacetate (4, 78.94 mg, 404.72 μmol) at 0° C. Then the mixture was at room temperature for 1 h. After completion of the reaction the solvent was removed under reduced pressure and the residue was suspended in water (30 mL). The precipitate was filtered and dried to get tert-butyl 2-[4-[1-(2,6-dioxo-3-piperidyl)-3-methyl-2-oxo-benzimidazol-5-yl]-3-fluoro-1-piperidyl]acetate (5, 150 mg, 314.87 μmol, 78% yield) as white solid.

LCMS (ES+): m/z 475.2 [M+H]⁺

### Step 4: 2-[4-[1-(2,6-dioxo-3-piperidyl)-3-methyl-2-oxo-benzimidazol-5-yl]-3-fluoro-1-piperidyl]acetic acid (6)

Into a 50 mL single-neck round-bottom flask containing a well-stirred solution of tert-butyl 2-[4-[1-(2,6-dioxo-3-piperidyl)-3-methyl-2-oxo-benzimidazol-5-yl]-3-fluoro-1-piperidyl]acetate (5, 140 mg, 292.08 μmol) in anhydrous DCM (5 mL) was added TFA (2.96 g, 25.96 mmol). After 5 h, the volatiles were removed under reduced pressure and the residue was triturated with with MTBE (50 mL), filtered and dried to get 2-[4-[1-(2,6-dioxo-3-piperidyl)-3-methyl-2-oxo-benzimidazol-5-yl]-3-fluoro-1-piperidyl]acetic acid (6, 135 mg, 247.87 μmol, 85% yield, TFA salt) as an off-white solid.

LCMS (ES+): m/z 419.2 [M+H]⁺

### tert-butyl (3R,4R)-4-[1-(2,6-dibenzyloxy-3-pyridyl)-3-methyl-2-oxo-benzimidazol-5-yl]-3-fluoro-piperidine-1-carboxylate (2a) and tert-butyl (3S,4S)-4-[1-(2,6-dibenzyloxy-3-pyridyl)-3-methyl-2-oxo-benzimidazol-5-yl]-3-fluoro-piperidine-1-carboxylate (2b)

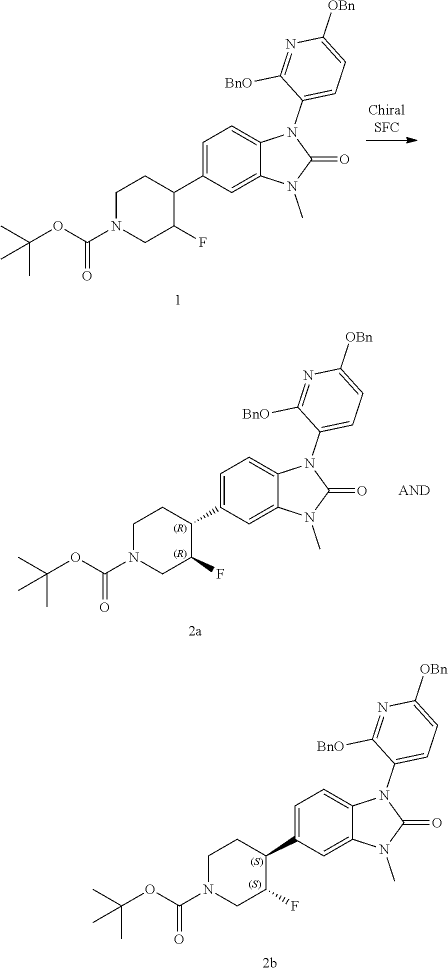

Separation method for Diastereomers: tert-butyl 4-[1-(2,6-dibenzyloxy-3-pyridyl)-3-methyl-2-oxo-benzimidazol-5-yl]-3-fluoro-piperidine-1-carboxylate (1, 1.4 g) was subjected to chiral SFC.

Method: Column Name: Chiralpak OX-H; FlowRate: 5 mL/min, Co-Solvent: 35%, Co-Solvent Name: 0.5% Isopropyl Amine in IPA; Outlet Pressure: 100 bar; Injected Volume: 10 μl, Temperature: 40° C.

Diastereomer 1: tert-butyl (3R,4R)-4-[1-(2,6-dibenzyloxy-3-pyridyl)-3-methyl-2-oxo-benzimidazol-5-yl]-3-fluoro-piperidine-1-carboxylate (520 mg, 804.68 μmol, fast eluting fraction) as an off-white solid.

Diastereomer 2: tert-butyl (3S,4S)-4-[1-(2,6-dibenzyloxy-3-pyridyl)-3-methyl-2-oxo-benzimidazol-5-yl]-3-fluoro-piperidine-1-carboxylate (480 mg, 743.83 μmol, late eluting fraction) as an off-white solid.

### 2-[4-[3-(2,6-dioxo-3-piperidyl)-1-methyl-indazol-6-yl]oxyphenyl]acetic acid (5)

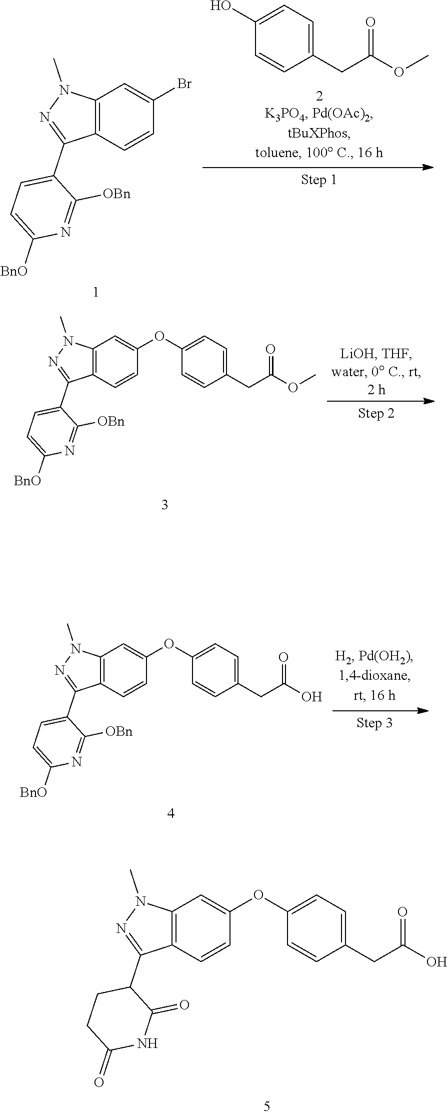 [SMILES: Cn1nc(-c2ccc(OCc3ccccc3)nc2OCc2ccccc2)c2ccc(Br)cc21 ; COC(=O)Cc1ccc(O)cc1 ; COC(=O)Cc1ccc(Oc2ccc3c(-c4ccc(OCc5ccccc5)nc4OCc4ccccc4)nn(C)c3c2)cc1 ; Cn1nc(-c2ccc(OCc3ccccc3)nc2OCc2ccccc2)c2ccc(Oc3ccc(CC(=O)O)cc3)cc21 ; "4" ; Cn1nc(C2CCC(=O)NC2=O)c2ccc(Oc3ccc(CC(=O)O)cc3)cc21]

### Step 1: methyl 2-[4-[3-(2,6-dibenzyloxy-3-pyridyl)-1-methyl-indazol-6-yl]oxyphenyl]acetate (3)

Into a 50 mL pressure tube containing a well-stirred solution of 6-bromo-3-(2,6-dibenzyloxy-3-pyridyl)-1-methyl-indazole (1, 1 g, 2.00 mmol) and methyl 2-(4-hydroxyphenyl)acetate (2, 664.18 mg, 4.00 mmol) in anhydrous toluene (10 mL) was added tripotassium phosphate (1.70 g, 7.99 mmol) at room temperature. The suspension was degassed by purging nitrogen gas for 10 min. Then, palladium(II) acetate (134.60 mg, 599.54 μmol) and t-Bu XPhos (254.59 mg, 599.54 μmol) were added and the reaction mixture was stirred at 100° C. After 16 h, the reaction mixture was filtered through Celite bed, filtrate was concentrated under reduced pressure. The crude compound was purified by flash silica gel column chromatography (20% EtOAC in pet ether) to obtain methyl 2-[4-[3-(2,6-dibenzyloxy-3-pyridyl)-1-methyl-indazol-6-yl]oxyphenyl]acetate (3, 600 mg, 799.12 μmol, 40% yield) as a yellow gummy mass.

LCMS (ES+): m/z 586.2 [M+H]⁺

### Step 2: 2-[4-[3-(2,6-dibenzyloxy-3-pyridyl)-1-methyl-indazol-6-yl]oxyphenyl]acetic acid (4)

Into a 50 mL single neck round-bottom flask containing a well-stirred solution of methyl 2-[4-[3-(2,6-dibenzyloxy-3-pyridyl)-1-methyl-indazol-6-yl]oxyphenyl]acetate (3, 600 mg, 799.12 μmol) in THF (5 mL) and water (3 mL) was added lithium hydroxide monohydrate (67.07 mg, 1.60 mmol) and the reaction mixture was stirred at ambient temperature. After 2 h, the reaction mixture was concentrated and the residue was neutralized with aq. 1.5 N HCl solution. The aqueous layer was extracted with EtOAc (2×50 mL). The combined organic layer was washed with water (50 mL), brine (25 mL) and dried over sodium sulfate. The solvent was evaporated to get 2-[4-[3-(2,6-dibenzyloxy-3-pyridyl)-1-methyl-indazol-6-yl]oxyphenyl]acetic acid (4, 550 mg, 724.13 μmol, 91% yield) as a yellow solid.

LCMS (ES+): m/z 572.2 [M+H]⁺

### Step 3: 2-[4-[3-(2,6-dioxo-3-piperidyl)-1-methyl-indazol-6-yl]oxyphenyl]acetic acid (5)

Into a 50 mL single-neck round-bottom flask containing a well-stirred solution of 2-[4-[3-(2,6-dibenzyloxy-3-pyridyl)-1-methyl-indazol-6-yl]oxyphenyl]acetic acid (4, 550 mg, 724.13 μmol) in 1,4-dioxane (10 mL) was added dihydroxypalladium (508.49 mg, 724.13 μmol, 20% purity) under nitrogen atmosphere at ambient temperature. Then the suspension was stirred under hydrogen atmosphere (bladder) at room temperature for 16 h. The reaction was filtered through Celite and washed with 1,4-dioxane (100 mL). The filtrate was concentrated under reduced pressure to get crude compound which was purified by reverse phase column chromatography [Silicycle C18 column; Mobile phase A: 0.1% TFA in water; Mobile phase B: MeCN] to obtain 2-[4-[3-(2,6-dioxo-3-piperidyl)-1-methyl-indazol-6-yl]oxyphenyl]acetic acid (5, 210 mg, 488.76 μmol, 68% yield) as an off-white solid.

LCMS (ES+): m/z 394.2 [M+H]⁺

### 2-[4-[1-(2,6-dibenzyloxy-3-pyridyl)-4-fluoro-3-methyl-2-oxo-benzimidazol-5-yl]phenyl]acetic acid (8)

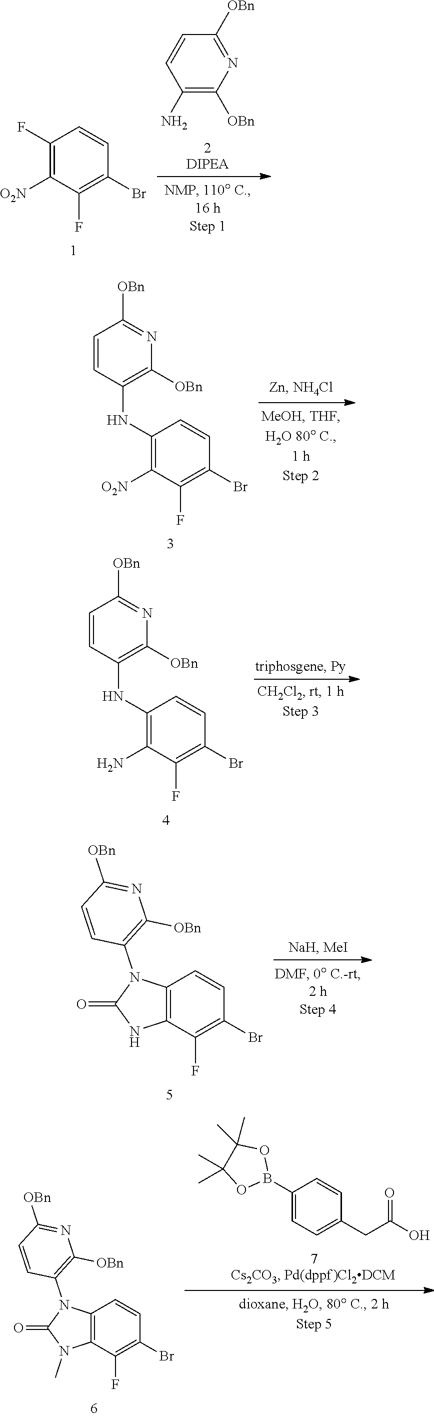

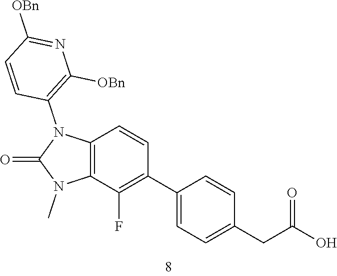 [SMILES: Cn1c(=O)n(-c2ccc(OCc3ccccc3)nc2OCc2ccccc2)c2ccc(-c3ccc(CC(=O)O)cc3)c(F)c21]

### Step 1: 2,6-dibenzyloxy-N-(4-bromo-3-fluoro-2-nitro-phenyl) 143yridine-3-amine (3)

Into a 100 mL sealed-tube containing a well-stirred solution of 1-bromo-2,4-difluoro-3-nitro-benzene (1, 5 g, 21.01 mmol) and 2,6-dibenzyloxypyridin-3-amine (2, 7.96 g, 25.21 mmol) in anhydrous NMP (30 mL) was added DIPEA (13.58 g, 105.05 mmol, 18.30 mL) at room temperature and the resulting mixture was heated at 110° C. for 16 h. The reaction mixture diluted with water (100 mL) and extracted with EtOAc (250 mL). Organic layer was washed with water (3×80 mL), dried over sodium sulfate, filtered and concentrated under reduced pressure to get crude material that was purified by flash silica gel column chromatography (100-200 mesh silica gel; 5% EtOAc in Pet ether) to afford 2,6-dibenzyloxy-N-(4-bromo-3-fluoro-2-nitro-phenyl)₁₄₃yridine-3-amine (3, 4.1 g, 6.91 mmol, 33% yield) as a red solid.

LCMS: m/z 524.0 [M+H]⁺

### Step 2: 4-bromo-N1-(2,4-dibenzyloxyphenyl)-3-fluoro-benzene-1,2-diamine (4)

Into a 250 mL three-neck round-bottom flask containing a well-stirred solution of 4-bromo-N-(2,4-dibenzyloxyphenyl)-3-fluoro-2-nitro-aniline (3, 5.7 g, 9.58 mmol) in THF (30 mL), methanol (25 mL) and water (10 mL) were added zinc dust (High grade material, 3.13 g, 47.92 mmol) and ammonium chloride (2.56 g, 47.92 mmol, 1.68 mL) at room temperature. The reaction mixture was heated at 80° C. for 1 h. The reaction mixture was filtered through Celite and washed with EtOAc (100 mL). The filtrate was diluted with water (100 mL) and extracted with EtOAc (2×100 mL). The combine organic layers were dried over sodium sulfate, filtered and concentrated under reduced pressure to afford 4-bromo-N1-(2,4-dibenzyloxyphenyl)-3-fluoro-benzene-1,2-diamine (4, 4.7 g, 8.46 mmol, 88% yield) as a brown oil.

LCMS (ES+): m/z 496.0 [M+H]⁺

### Step 3: 6-bromo-3-(2,4-dibenzyloxyphenyl)-7-fluoro-1H-benzimidazol-2-one (5)

Into a 250 mL three-neck round-bottom flask containing a well-stirred solution of 4-bromo-N1-(2,4-dibenzyloxyphenyl)-3-fluoro-benzene-1,2-diamine (4, 4.7 g, 8.46 mmol) in anhydrous DCM (30 mL) was added triphosgene (5.02 g, 16.92 mmol) and the resulting mixture was cooled to 0° C. Pyridine (3.35 g, 42.31 mmol, 3.42 mL) in anhydrous DCM (10 mL) was added and stirring was continued at room temperature for 2 h. The reaction mixture was quenched with cold water at 0° C. and the organic layer was extracted with EtOAc (2×100 mL). Combined organic layers were dried over anhydrous Na₂SO₄, filtered and concentrated under reduced pressure to get the crude compound that was purified by flash silica gel column chromatography (230-400 mesh silica gel; 40% EtOAc in Pet ether) to afford 6-bromo-3-(2,4-dibenzyloxyphenyl)-7-fluoro-1H-benzimidazol-2-one (5, 3.5 g, 5.20 mmol, 61% yield) as a brown solid.

LCMS (ES+): m/z 520.8 [M+H]⁺

### Step 4: 5-bromo-1-(2,6-dibenzyloxy-3-pyridyl)-4-fluoro-3-methyl-benzimidazol-2-one (6)

Into a 100 mL single-neck round-bottom flask containing a well-stirred solution of 6-bromo-3-(2,6-dibenzyloxy-3-pyridyl)-7-fluoro-1H-benzimidazol-2-one (5, 3.5 g, 6.73 mmol) in DMF (20 mL) was added sodium hydride (60% dispersion in mineral oil, 386.59 mg, 10.09 mmol, 60% purity) at 0° C. The reaction mixture stirred at 25° C. for 30 min and then iodomethane (436.65 mg, 3.08 mmol, 191.51 μL) was added at 0° C. After 2 h at room temperature, the mixture was quenched with saturated ammonium chloride solution (20 mL) and extracted with ethyl acetate (2×150 mL). The combined organic layers were dried over Na₂SO₄, filtered, concentrated to obtain the crude material that was purified by silica gel column chromatography (100-200 mesh silica gel; 40-50% EtOAc in pet ether) to afford 5-bromo-1-(2,6-dibenzyloxy-3-pyridyl)-4-fluoro-3-methyl-benzimidazol-2-one (6, 1.9 g, 3.00 mmol, 45% yield) as an off-white solid.

LCMS (ES+): m/z 536.0 [M+H]⁺

### Step 5: 2-[4-[1-(2,6-dibenzyloxy-3-pyridyl)-4-fluoro-3-methyl-2-oxo-benzimidazol-5-yl]phenyl]acetic acid (8)

Into a 100 mL sealed-tube containing a well-stirred solution of 5-bromo-1-(2,6-dibenzyloxy-3-pyridyl)-4-fluoro-3-methyl-benzimidazol-2-one (6, 500 mg, 935.67 μmol) and 2-[4-(4,4,5,5-tetramethyl-1,3,2-dioxaborolan-2-yl)phenyl]acetic acid (7, 367.87 mg, 1.40 mmol) in 1,4-dioxane (5 mL) and water (0.5 mL) were added cesium carbonate (914.58 mg, 2.81 mmol) at room temperature. The reaction mixture was purged with nitrogen gas for 10 min. Later [1,1′-bis(diphenylphosphino)ferrocene]dichloropalladium(II), complex with dichloromethane (114.53 mg, 140.35 μmol) was added and reaction mixture was heated at 90° C. for 2 h. The reaction mixture was filtered through Celite and washed thoroughly with EtOAc (250 mL). The filtrate was washed with water (100 mL), dried over Na₂SO₄, filtered and concentrated under reduced pressure to get the crude material that was purified by reverse-phase column chromatography eluted [Column: Redisep C18-120 g; Mobile Phase A: 0.1% TFA in water and Mobile Phase B: CH₃CN] to afford 2-[4-[1-(2,6-dibenzyloxy-3-pyridyl)-4-fluoro-3-methyl-2-oxo-benzimidazol-5-yl]phenyl]acetic acid (8, 430 mg, 625.37 μmol, 67% yield) as an off-white solid.

LCMS (ES+): m/z 590.2 [M+H]⁺

### 2-(4-((3-(2,6-bis(benzyloxy)145yridine-3-yl)-1-methyl-1H-indazol-6-yl)amino)-3-fluorophenyl)acetic acid (6)

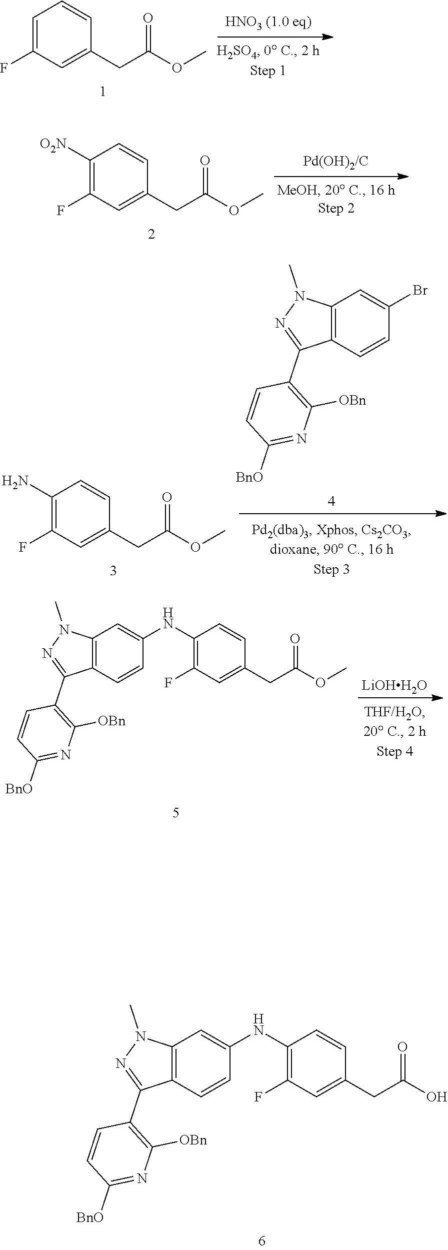

### Step 1: methyl 2-(3-fluoro-4-nitro-phenyl)acetate (2)

To a solution of methyl 2-(3-fluorophenyl)acetate (1, 20 g, 118.93 mmol, 1 eq) in H₂SO₄ (34 mL) was added HNO₃ (7.5 mL, 68% purity, 1 eq) slowly at 0° C. over 1 h in a three-neck flask under N₂. The reaction mixture was stirred at 0° C. for 1 h. Then the reaction mixture was dropped into ice-water (200 mL) slowly at 0° C. A large quantity of yellow precipitate was formed. The mixture was filtered and the filter cake was dried under vacuum. The residue was purified on automated flash chromatography system (ethyl acetate/petroleum ether from 0:1 to 4:6) to afford methyl 2-(3-fluoro-4-nitro-phenyl)acetate (2, 2.55 g, 11.96 mmol, 10% yield) as a yellow solid.

¹H NMR (400 MHz, DMSO-d₆) δ=8.13 (t, J=8.4 Hz, 1H), 7.54 (dd, J=1.6, 12.4 Hz, 1H), 7.37 (dd, J=0.8, 8.4 Hz, 1H), 3.90 (s, 2H), 3.64 (s, 3H).

### Step 2: methyl 2-(4-amino-3-fluoro-phenyl)acetate (3)

To a solution of ethyl 2-(3-fluoro-4-nitro-phenyl)acetate (2, 2.55 g, 11.96 mmol, 1 eq) in MeOH (30 mL) was added Pd(OH)₂/C (500 mg, 10% purity). The reaction mixture was stirred at 20° C. for 16 h under H₂ atmosphere (15 psi). The reaction mixture was filtered through Celite. The filtrate was concentrated under vacuum. The residue was purified on automated flash chromatography system (ethyl acetate/petroleum ether from 0:1 to 4:6) to afford methyl 2-(4-amino-3-fluoro-phenyl)acetate (3, 1.8 g, 9.73 mmol, 81% yield) as a colorless oil.

LCMS (ESI): m/z 183.9 [M+H]⁺

### Step 3: methyl 2-[4-[[3-(2,6-dibenzyloxy-3-pyridyl)-1-methyl-indazol-6-yl]amino]-3-fluoro-phenyl]acetate (5)

To a solution of 6-bromo-3-(2,6-dibenzyloxy-3-pyridyl)-1-methyl-indazole (4, 1 g, 2.00 mmol, 1 eq) and methyl 2-(4-amino-3-fluoro-phenyl)acetate (3, 440 mg, 2.40 mmol, 1.2 eq) in 1,4-dioxane (20 mL) were added Cs₂CO₃ (2.00 g, 6.14 mmol, 3 eq), Pd₂(dba)₃ (199.47 mg, 217.83 μmol, 0.1 eq) and Xphos (200.00 mg, 419.54 μmol, 0.2 eq) under N₂. The reaction mixture was stirred at 90° C. for 16 h. The reaction mixture was filtered and the filtrate was concentrated under vacuum. The residue was purified on automated flash chromatography system (ethyl acetate/petroleum ether from 0:1 to 1:1) to afford methyl 2-[4-[[3-(2,6-dibenzyloxy-3-pyridyl)-1-methyl-indazol-6-yl]amino]-3-fluoro-phenyl]acetate (5, 960 mg, 1.31 mmol, 65% yield) as a yellow oil.

LCMS (ESI): m/z 603.3 [M+H]⁺

### Step 4: 2-(4-((3-(2,6-bis(benzyloxy)pyridin-3-yl)-1-methyl-1H-indazol-6-yl)amino)-3-fluorophenyl)acetic acid (6)

To a solution of methyl 2-[4-[[3-(2,6-dibenzyloxy-3-pyridyl)-1-methyl-indazol-6-yl]amino]-3-fluoro-phenyl]acetate (5, 1 g, 1.33 mmol, 1 eq) in THF (15 mL) was added a solution of LiOH H₂O (167.38 mg, 3.98 mmol, 3 eq) in H₂O (5 mL). After stirring at 20° C. for 2 h, the reaction mixture was concentrated under vacuum to remove THF. The residue was acidified to pH˜5. A large quantity of yellow precipitate was formed. The mixture was filtered and the filter cake was dried under vacuum. The residue was purified by reversed phase flash chromatography (flow: 50 mL/min; gradient: from 0-70% water (0.1% formic acid) in MeCN) to afford 2-(4-((3-(2,6-bis(benzyloxy)pyridin-3-yl)-1-methyl-1H-indazol-6-yl)amino)-3-fluorophenyl)acetic acid (6, 0.7 g, 1.17 mmol, 88% yield) as a yellow solid.

LCMS (ESI): m/z 589.3 [M+H]⁺

### 2-(4-((1-(2,6-bis(benzyloxy)pyridin-3-yl)-3-methyl-2-oxo-2,3-dihydro-1H-benzo[d]imidazol-5-yl)amino)-1H-pyrazol-1-yl)acetic acid (5)

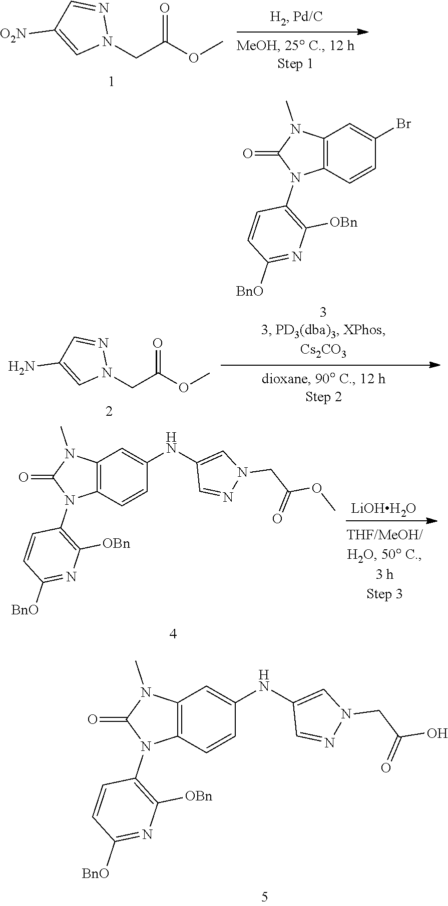

### Step 1: methyl 2-(4-amino-1H-pyrazol-1-yl)acetate (2)

To a solution of methyl 2-(4-nitro-1H-pyrazol-1-yl)acetate (1, 3 g, 16.20 mmol) in MeOH (100 mL) was added Pd/C (0.2 g) under H₂ atmosphere. The suspension was degassed and purged with H₂ for 3 times. Then the mixture was stirred at 25° C. for 12 hrs. The reaction mixture was filtered and the filtrate was concentrated under reduced pressure to give crude product. methyl 2-(4-amino-1H-pyrazol-1-yl)acetate (2, 2.5 g, 16.11 mmol, 99% yield) as brown oil.

LCMS (ESI): m/z 156.2 [M+H]⁺

### Step 2: methyl 2-(4-((1-(2,6-bis(benzyloxy)pyridin-3-yl)-3-methyl-2-oxo-2,3-dihydro-1H-benzo[d]imidazol-5-yl)amino)-1H-pyrazol-1-yl)acetate (4)

To a solution of methyl 2-(4-amino-1H-pyrazol-1-yl)acetate (2, 450.69 mg, 2.90 mmol) and 1-(2,6-bis(benzyloxy)pyridin-3-yl)-5-bromo-3-methyl-1H-benzo[d]imidazol-2 (3H)-one (3, 1 g, 1.94 mmol) in dioxane (10 mL) was added cesium hydroxide hydrate (975.60 mg, 5.81 mmol, 521.71 μL), ditert-butyl-[2-[2,4,6-tri(propan-2-yl)phenyl]phenyl]phosphane (164.47 mg, 387.31 μmol) and (1E,4E)-1,5-diphenylpenta-1,4-dien-3-one; palladium (177.33 mg, 193.65 μmol) under N₂ atmosphere. The suspension was degassed and purged with N₂ for 3 times. Then the mixture was stirred at 90° C. for 12 hrs. The reaction was washed with water (10 mL) and extracted with ethyl acetate 30 mL (10 mL*3). The combined organic layers were dried over anhydrous sodium sulfate, filtered and concentrated under reduced pressure to give a residue. The residue was purified by flash silica gel chromatography (ISCO®; 20 g SepaFlash® Silica Flash Column, Eluent of 0-100% EA/PE) and the eluent was concentrated to give methyl 2-(4-((1-(2,6-bis(benzyloxy)pyridin-3-yl)-3-methyl-2-oxo-2,3-dihydro-1H-benzo[d]imidazol-5-yl)amino)-1H-pyrazol-1-yl)acetate (4, 0.4 g, 677.25 μmol, 35% yield) as yellow oil.

LCMS (ESI): m/z 591.0 [M+H]⁺

### Step 3: 2-(4-((1-(2,6-bis(benzyloxy)pyridin-3-yl)-3-methyl-2-oxo-2,3-dihydro-1H-benzo[d]imidazol-5-yl)amino)-1H-pyrazol-1-yl)acetic acid (5)

To a solution of methyl 2-(4-((1-(2,6-bis(benzyloxy)pyridin-3-yl)-3-methyl-2-oxo-2,3-dihydro-1H-benzo[d]imidazol-5-yl)amino)-1H-pyrazol-1-yl)acetate (4, 0.4 g, 677.25 μmol) in Ethanol (1.5 mL), Water (1.5 mL) and THF (1.5 mL) was added Lithium hydroxide, monohydrate (142.10 mg, 3.39 mmol, 94.10 μL), then the mixture was stirred at 50° C. for 3 hrs. The mixture was concentrated and extracted with ethyl acetate 12 mL (4 mL*3). The combined organic layers were dried over anhydrous sodium sulfate, filtered and concentrated under reduced pressure to give a residue. The residue was purified by reversed phase (0.1% FA) and concentrated to give 2-(4-((1-(2,6-bis(benzyloxy)pyridin-3-yl)-3-methyl-2-oxo-2,3-dihydro-1H-benzo[d]imidazol-5-yl)amino)-1H-pyrazol-1-yl)acetic acid (5, 0.25 g, 433.58 μmol, 64% yield) as yellow solid.

LCMS (ESI): m/z 576.9 [M+H]⁺

### 2-[1-[1-(2,6-dioxo-3-piperidyl)-6-fluoro-3-methyl-2-oxo-benzimidazol-5-yl]-4-piperidyl]acetic acid (10)

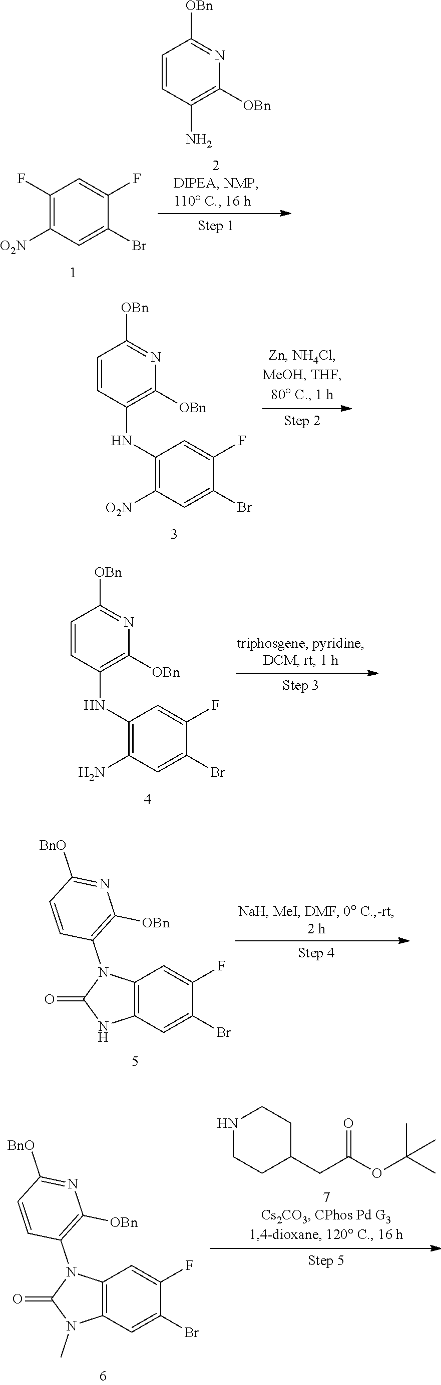

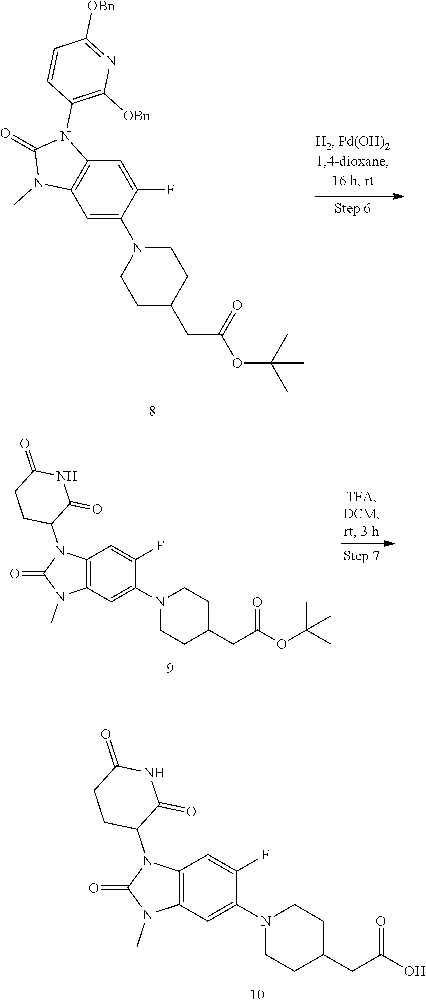 [SMILES: Cn1c(=O)n(-c2ccc(OCc3ccccc3)nc2OCc2ccccc2)c2cc(F)c(N3CCC(CC(=O)OC(C)(C)C)CC3)cc21 ; Cn1c(=O)n(C2CCC(=O)NC2=O)c2cc(F)c(N3CCC(CC(=O)OC(C)(C)C)CC3)cc21 ; Cn1c(=O)n(C2CCC(=O)NC2=O)c2cc(F)c(N3CCC(CC(=O)O)CC3)cc21]

### Step 1: 2,6-dibenzyloxy-N-(4-bromo-5-fluoro-2-nitro-phenyl)pyridin-3-amine (3)

Into a 100 mL pressure tube containing a well-stirred solution of 1-bromo-2,4-difluoro-5-nitro-benzene (1, 3 g, 12.61 mmol) and 2,6-dibenzyloxypyridin-3-amine (2, 3.86 g, 12.61 mmol) in anhydrous NMP (30 mL) was added DIPEA (8.15 g, 63.03 mmol, 10.98 mL) at room temperature. The mixture was then stirred at 110° C. for 16 h. The reaction mixture was diluted with ethyl acetate (150 mL) and the organic layer was washed with water (3×200 mL), dried over anhydrous Na₂SO₄ and filtered. The filtrate was concentrated under reduced pressure and the crude compound was purified by flash silica gel (230-400 mesh) column chromatography (8% EtOAc in pet ether) to afford 2,6-dibenzyloxy-N-(4-bromo-5-fluoro-2-nitro-phenyl)pyridin-3-amine (3, 2.3 g, 3.95 mmol, 31% yield).

¹HNMR (400 MHz, DMSO-d₆): δ 9.26 (s, 1H), 8.48 (d, J=7.20 Hz, 1H), 7.52 (d, J=8.00 Hz, 1H), 7.48-7.29 (m, 10H), 6.55 (d, J=10.40 Hz, 1H), 6.50 (d, J=8.40 Hz, 1H), 5.41 (s, 2H), 5.39 (s, 2H).

### Step 2: 4-bromo-N1-(2,6-dibenzyloxy-3-pyridyl)-5-fluoro-benzene-1,2-diamine (4)

Into a 250 mL single neck round bottom flask containing a well-stirred solution of 2,6-dibenzyloxy-N-(4-bromo-5-fluoro-2-nitro-phenyl)pyridin-3-amine (3, 2.30 g, 3.96 mmol) in methanol (30 mL), THF (45 mL) and water (5 mL) were added zinc powder (2.59 g, 39.55 mmol) and ammonium chloride (2.12 g, 39.55 mmol) at ambient temperature. The resulting suspension was stirred at 80° C. for 1 h. The reaction mixture was filtered through Celite and washed with ethyl acetate (150 mL). The filtrate was washed with water (3×100 mL), dried over anhydrous Na₂SO₄, filtered and concentrated under reduced pressure to get crude 4-bromo-N1-(2,6-dibenzyloxy-3-pyridyl)-5-fluoro-benzene-1,2-diamine (4, 2.19 g, 3.51 mmol, 89% yield) as brown gummy liquid.

LCMS (ES+): m/z 494.0 [M+H]⁺

### Step 3: 6-bromo-3-(2,6-dibenzyloxy-3-pyridyl)-5-fluoro-1H-benzimidazol-2-one (5)

Into a 250 mL single-neck round-bottom flask containing a well-stirred solution of 4-bromo-N1-(2,6-dibenzyloxy-3-pyridyl)-5-fluoro-benzene-1,2-diamine (4, 2.19 g, 3.52 mmol) in anhydrous DCM (30 mL) were added triphosgene (2.09 g, 7.03 mmol) and pyridine (1.39 g, 17.58 mmol, 1.42 mL) at 0° C. After 1 h, the reaction mixture was diluted with DCM (100 mL) and washed with water (2×100 mL), dried over anhydrous Na₂SO₄ and filtered. The filtrate was concentrated under reduced pressure and the crude compound was purified by flash silica gel (230-400 mesh) column chromatography (34% EtOAc in pet ether) to afford 6-bromo-3-(2,6-dibenzyloxy-3-pyridyl)-5-fluoro-1H-benzimidazol-2-one (5, 1.23 g, 1.88 mmol, 53% yield).

LCMS (ES+): m/z 522.0 [M+H]⁺

### Step 4: 5-bromo-1-(2,6-dibenzyloxy-3-pyridyl)-6-fluoro-3-methyl-benzimidazol-2-one (6)

Into a 100 mL single neck round bottom flask containing a well-stirred solution of 6-bromo-3-(2,6-dibenzyloxy-3-pyridyl)-5-fluoro-1H-benzimidazol-2-one (5, 1.22 g, 1.87 mmol) in anhydrous DMF (20 mL) was added sodium hydride, 60% dispersion in mineral oil (107.33 mg, 2.80 mmol, 60% purity) at 0° C. The reaction mixture was stirred at room temperature for 30 min. Then the reaction mixture was cooled to 0° C. and iodomethane (530.14 mg, 3.73 mmol, 232.52 μL) was added. The reaction mixture was stirred at room temperature for 2 h. The reaction was quenched with saturated ammonium chloride solution. The mixture was extracted with ethyl acetate (2×50 mL). The combined organic layer was dried over anhydrous Na₂SO₄, filtered. The solvent was removed under reduced pressure and the crude compound was purified by flash silica gel (230-400 mesh) column chromatography (31% EtOAc in pet ether) to afford 5-bromo-1-(2,6-dibenzyloxy-3-pyridyl)-6-fluoro-3-methyl-benzimidazol-2-one (6, 1 g, 1.80 mmol, 96% yield) as an off-white solid.

LCMS (ES+): m/z 536.0 [M+H]⁺

### Step 5: tert-butyl 2-[1-[1-(2,6-dibenzyloxy-3-pyridyl)-6-fluoro-3-methyl-2-oxo-benzimidazol-5-yl]-4-piperidyl]acetate (8)

Into a 50 mL pressure tube containing a well-stirred solution of 5-bromo-1-(2,6-dibenzyloxy-3-pyridyl)-6-fluoro-3-methyl-benzimidazol-2-one (6, 370 mg, 665.67 μmol) and tert-butyl 2-(4-piperidyl)acetate (7, 265.32 mg, 1.33 mmol) in anhydrous 1,4-dioxane (8 mL) was added cesium carbonate (650.66 mg, 2.00 mmol) at room temperature. The mixture was degassed by bubbling of nitrogen gas for 10 min. Then, CPhos Pd G3 (53.68 mg, 66.57 μmol) was added and the reaction mixture was heated at 120° C. After 16 h, the reaction mixture was filtered through Celite and the filtrate was concentrated under reduced pressure. The crude compound was purified by flash silica gel (230-400 mesh) column chromatography (30% EtOAc in pet ether) to afford tert-butyl 2-[1-[1-(2,6-dibenzyloxy-3-pyridyl)-6-fluoro-3-methyl-2-oxo-benzimidazol-5-yl]-4-piperidyl]acetate (8, 327 mg, 175.74 μmol, 26% yield) as a pale yellow gummy liquid.

LCMS (ES+): m/z 653.2 [M+H]⁺

### Step 6: tert-butyl 2-[1-[1-(2,6-dioxo-3-piperidyl)-6-fluoro-3-methyl-2-oxo-benzimidazol-5-yl]-4-piperidyl]acetate (9)

Into a 50 mL single-neck round-bottom flask containing a well-stirred solution of tert-butyl 2-[1-[1-(2,6-dibenzyloxy-3-pyridyl)-6-fluoro-3-methyl-2-oxo-benzimidazol-5-yl]-4-piperidyl]acetate (8, 327 mg, 175.74 μmol, crude) in anhydrous 1,4-dioxane (4.5 mL) was added palladium hydroxide on carbon, 20 wt. % (300 mg, 427.24 μmol, 20% purity) at room temperature. The suspension was stirred at room temperature under hydrogen atmosphere for 16 h. The reaction mixture was filtered through Celite and the filtrate was concentrated under reduced pressure. The crude compound was purified by reverse phase column chromatography [Purification method: Silicycle C18 column; Mobile phase A: 0.1% Formic acid in water; Mobile phase B: MeCN] to get tert-butyl 2-[1-[1-(2,6-dioxo-3-piperidyl)-6-fluoro-3-methyl-2-oxo-benzimidazol-5-yl]-4-piperidyl]acetate (9, 70 mg, 147.21 μmol, 84% yield) as a colorless solid.

LCMS (ES+): m/z 475.2 [M+H]⁺

### Step 7: 2-[1-[1-(2,6-dioxo-3-piperidyl)-6-fluoro-3-methyl-2-oxo-benzimidazol-5-yl]-4-piperidyl]acetic acid (10)

Into a 25 mL single-neck round-bottom flask containing a well-stirred solution of tert-butyl 2-[1-[1-(2,6-dioxo-3-piperidyl)-6-fluoro-3-methyl-2-oxo-benzimidazol-5-yl]-4-piperidyl]acetate (9, 65 mg, 136.69 μmol) in anhydrous DCM (1.5 mL) was added trifluoroacetic acid (1.04 g, 9.09 mmol, 0.7 mL). After 3 h, the solvent was removed under reduced pressure to get 2-[1-[1-(2,6-dioxo-3-piperidyl)-6-fluoro-3-methyl-2-oxo-benzimidazol-5-yl]-4-piperidyl]acetic acid (10, 65 mg, 121.71 μmol, 89% yield, TFA salt) as off white solid.

LCMS (ES+): m/z 419.2 [M+H]⁺

### 2-[4-[1-(2,6-dibenzyloxy-3-pyridyl)-6-fluoro-3-methyl-2-oxo-benzimidazol-5-yl]phenyl]acetic acid (3)

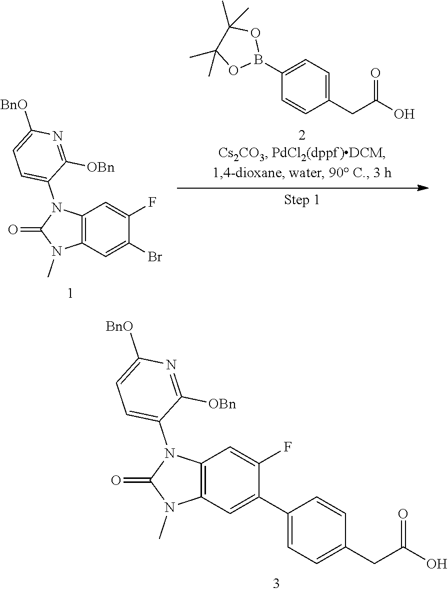 [SMILES: Cn1c(=O)n(-c2ccc(OCc3ccccc3)nc2OCc2ccccc2)c2cc(F)c(Br)cc21 ; CC1(C)OB(c2ccc(CC(=O)O)cc2)OC1(C)C ; Cn1c(=O)n(-c2ccc(OCc3ccccc3)nc2OCc2ccccc2)c2cc(F)c(-c3ccc(CC(=O)O)cc3)cc21]

### Step 1: 2-[4-[1-(2,6-dibenzyloxy-3-pyridyl)-6-fluoro-3-methyl-2-oxo-benzimidazol-5-yl]phenyl]acetic acid (3)

Into a 25 mL pressure tube containing a well-stirred solution of 5-bromo-1-(2,6-dibenzyloxy-3-pyridyl)-6-fluoro-3-methyl-benzimidazol-2-one (1, 600 mg, 1.06 mmol) and 2-[4-(4,4,5,5-tetramethyl-1,3,2-dioxaborolan-2-yl)phenyl]acetic acid (2, 304.30 mg, 1.16 mmol) in 1,4-dioxane (3.0 mL) and water (0.3 mL) was added cesium carbonate (687.76 mg, 2.11 mmol) and the suspension was degassed by bubbling nitrogen gas for 5 min. Then, [1,1′-bis(diphenylphosphino)ferrocene]dichloropalladium(II), complex with dichloromethane (172.29 mg, 211.09 μmol) was added and reaction mixture was stirred at 90° C. After 3 h, the reaction mixture was filtered through Celite and washed with ethyl acetate (100 mL). The filtrate was concentrated under reduced pressure to get the crude compound, which was purified by reverse phase column chromatography [Siliasep C18 60 g, Mobile phase A: 0.1% TFA in water; Mobile phase B: MeCN] to obtain 2-[4-[1-(2,6-dibenzyloxy-3-pyridyl)-6-fluoro-3-methyl-2-oxo-benzimidazol-5-yl]phenyl]acetic acid (3, 400 mg, 521.63 μmol, 49% yield) as a pale yellow solid.

LCMS (ES+): m/z 590.2 [M+H]⁺

### 2-(4-(1-(2,6-bis(benzyloxy)pyridin-3-yl)-3-(2-methoxyethyl)-2-oxo-2,3-dihydro-1H-benzo[d]imidazol-5-yl)phenyl)acetic acid (6)

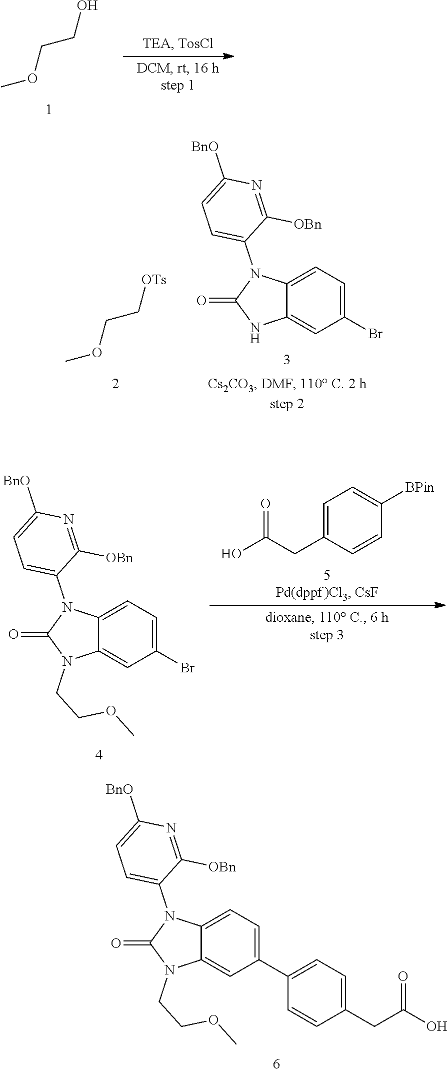

### Step 1: 2-methoxyethyl 4-methylbenzenesulfonate (2)

To a solution of 4-methylbenzene-1-sulfonyl chloride (9 g, 47.21 mmol, 1 eq) and triethylamine (14.33 g, 141.62 mmol, 19.74 mL, 3 eq) in DCM (30 mL) was added 2-methoxyethanol (1, 4.67 g, 61.37 mmol, 4.84 mL, 1.3 eq dropwise at 25° C. The resulting mixture was stirred at 25° C. for 16 h. The reaction mixture was diluted with DCM (50 mL) and washed with water (50 mL) followed by brine (50 mL) and then dried over Na₂SO₄, filtered and concentrated in vacuo. The residue was purified by column chromatography on silica gel (petroleum ether/ethyl acetate=10/1) to give 2-methoxyethyl 4-methylbenzenesulfonate (2, 6.5 g, 27.94 mmol, 59% yield) as a colorless oil.

LCMS (ESI): m/z 231.1 [M+H]⁺

¹H NMR (400 MHz, CDCl3) δ=7.84-7.78 (m, 2H), 7.35 (d, J=8.0 Hz, 2H), 4.19-4.14 (m, 2H), 3.61-3.56 (m, 2H), 3.32 (s, 3H), 2.45 (s, 3H).

### Step 2: 1-(2,6-bis(benzyloxy)pyridin-3-yl)-5-bromo-3-(2-methoxyethyl)-1H-benzo[d]imidazol-2 (3H)-one (4)

To a solution of 1-(2,6-bis(benzyloxy)pyridin-3-yl)-5-bromo-1H-benzo[d]imidazol-2 (3H)-one (3, 1.8 g, 3.58 mmol, 1 eq) and 2-methoxyethyl 4-methylbenzenesulfonate (2, 1.24 g, 5.37 mmol, 1.5 eq in DMF (15 mL) was added cesium carbonate (3.50 g, 10.75 mmol, 3 eq). The resulting mixture was stirred at 110° C. for 2 h. After the reaction mixture was cooled to room temperature, etOAc (40 mL) and water (40 mL) were added and layers were separated. The aqueous phase was extracted with EtOAc (40 mL×2). Combined extracts were washed with brine (60 mL), dried over Na₂SO₄, filtered, and concentrated under vacuum. The residue was purified on automated flash chromatography system (ethyl acetate/petroleum ether from 0:1 to 9:1) to give 1-(2,6-bis(benzyloxy)pyridin-3-yl)-5-bromo-3-(2-methoxyethyl)-1H-benzo[d]imidazol-2 (3H)-one (4, 2.1 g, 3.52 mmol, 98% yield) as a khaki solid.

LCMS (ESI): m/z 562.1 [M+H]⁺

### Step 3: 2-(4-(1-(2,6-bis(benzyloxy)pyridin-3-yl)-3-(2-methoxyethyl)-2-oxo-2,3-dihydro-1H-benzo[d]imidazol-5-yl)phenyl)acetic acid (6)

To a solution of 2-(4-(4,4,5,5-tetramethyl-1,3,2-dioxaborolan-2-yl)phenyl)acetic acid (5, 1.05 g, 4.01 mmol, 1.5 eq) and 5-bromo-1-(2,6-dibenzyloxy-3-pyridyl)-3-(2-methoxyethyl)benzimidazol-2-one (4, 1.5 g, 2.68 mmol, 1 eq) in dioxane (20 mL) were added cesium fluoride (1.22 g, 8.03 mmol, 3 eq) and Pd(dppf)C12 (195.84 mg, 267.65 μmol, 0.1 eq) under N₂ atmosphere. The mixture was stirred at 110° C. for 16 h. The resulting mixture was filtered through a pad of silica gel, and the filtrate was concentrated under reduced pressure. The residue was dissolved in MeOH (10 mL) and acidized with formic acid to pH get 5-6. The solution was purified by reversed phase column (0.1% FA in water/acetonitrile) and concentrated under reduced pressure to remove ACN. The aqueous phase was extracted with DCM (60 mL×2). The combined organic extracts were washed with brine (20 mL) and then dried over Na₂SO₄, filtered and concentrated in vacuo to give 2-(4-(1-(2,6-bis(benzyloxy)pyridin-3-yl)-3-(2-methoxyethyl)-2-oxo-2,3-dihydro-1H-benzo[d]imidazol-5-yl)phenyl)acetic acid (6, 680 mg, 945.45 μmol, 35% yield, formic acid salt) as a black foam.

LCMS (ES+): m/z 616.6 [M+H]⁺

### 2-[1-[1-(2,6-dioxo-3-piperidyl)-4-fluoro-3-methyl-2-oxo-benzimidazol-5-yl]-4-piperidyl]acetic acid (10)

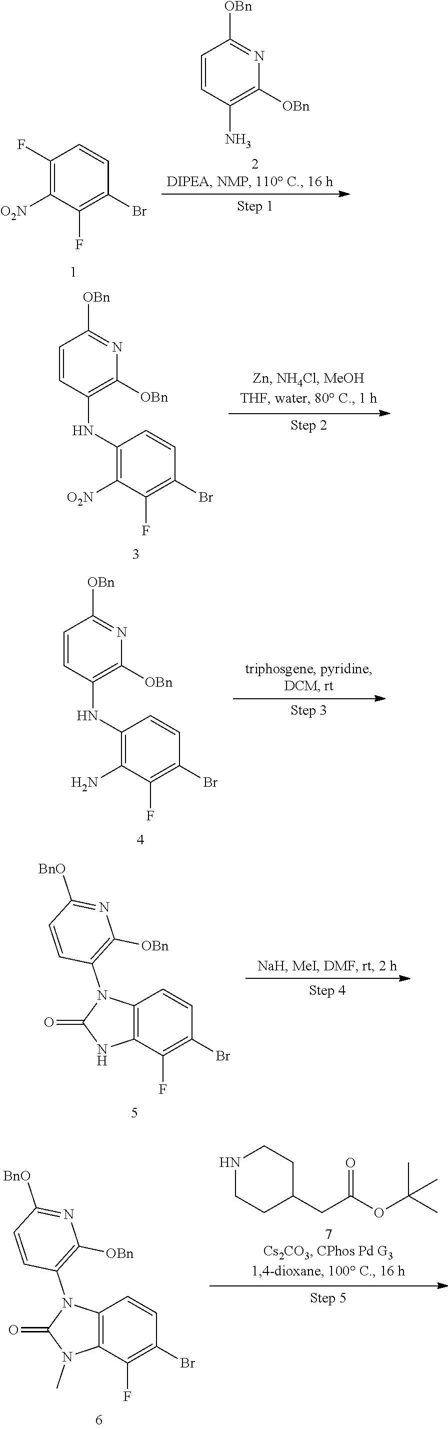

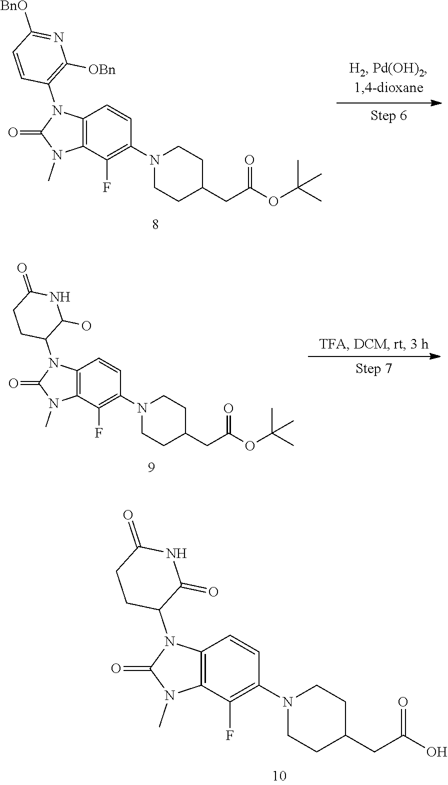 [SMILES: Cn1c(=O)n(-c2ccc(OCc3ccccc3)nc2OCc2ccccc2)c2ccc(N3CCC(CC(=O)OC(C)(C)C)CC3)c(F)c21 ; Cn1c(=O)n(C2CCC(=O)NC2O)c2ccc(N3CCC(CC(=O)OC(C)(C)C)CC3)c(F)c21 ; Cn1c(=O)n(C2CCC(=O)NC2=O)c2ccc(N3CCC(CC(=O)O)CC3)c(F)c21]

### Step 1: 2,6-dibenzyloxy-N-(4-bromo-3-fluoro-2-nitro-phenyl)pyridin-3-amine (3)

Into a 10 mL pressure tube containing a well-stirred solution of 1-bromo-2,4-difluoro-3-nitro-benzene (1, 3 g, 12.61 mmol) and 2,6-dibenzyloxypyridin-3-amine (2, 3.86 g, 12.61 mmol) in anhydrous NMP (25 mL) was added DIPEA (8.15 g, 63.03 mmol, 10.98 mL) and the reaction mixture was heated at 110° C. After 16 h, the reaction, reaction mixture diluted with water (60 mL) and extracted with ethyl acetate (2×150 mL). The organic layer was washed with water (3×60 mL), dried over sodium sulfate and filtered. The filtrate was concentrated under reduced pressure and the crude compound was purified by flash silica gel column chromatography (4% EtOAc in pet ether) to afford 2,6-dibenzyloxy-N-(4-bromo-3-fluoro-2-nitro-phenyl)pyridin-3-amine (3, 1.5 g, 2.50 mmol, 20% yield) as a reddish solid.

LCMS (ES+): m/z 526.0 [M+H]⁺

### Step 2: 4-bromo-N1-(2,4-dibenzyloxyphenyl)-3-fluoro-benzene-1,2-diamine (4)

Into a 250 mL three neck round bottom flask containing a well-stirred solution of 4-bromo-N-(2,4-dibenzyloxyphenyl)-3-fluoro-2-nitro-aniline (3, 1.54 g, 2.58 mmol) in THF (30 mL), methanol (25 mL) and water (10 mL) were added zine powder (843.38 mg, 12.90 mmol) and ammonium chloride (689.90 mg, 12.90 mmol, 450.91 μL) at room temperature. The reaction mixture was stirred at 80° C. for 1 h. The reaction mixture was filtered through Celite and washed with ethyl acetate (100 mL). The filtrate was concentrated to dryness, diluted with ethyl acetate (200 mL) and dried over anhydrous Na₂SO₄. The solvent was removed under reduced pressure to get crude 4-bromo-N1-(2,4-dibenzyloxyphenyl)-3-fluoro-benzene-1,2-diamine (4, 1.4 g, 2.48 mmol, 96% yield) as a brown colored thick mass.

LCMS (ES+): m/z 494.0 [M+H]⁺

### Step 3: 6-bromo-3-(2,4-dibenzyloxyphenyl)-7-fluoro-1H-benzimidazol-2-one (5)

Into a 250 mL single-neck round-bottom flask containing well-stirred solution of 4-bromo-N1-(2,4-dibenzyloxyphenyl)-3-fluoro-benzene-1,2-diamine (4, 1.4 g, 2.48 mmol) in anhydrous DCM (50 mL) were added triphosgene (1.47 g, 4.96 mmol) and stirred for 5 min. Then a solution of pyridine (981.55 mg, 12.41 mmol, 1.00 mL) in anhydrous DCM (10 mL) was added at 0° C. The reaction mixture was stirred at room temperature for 1 h. The reaction mixture was diluted with DCM (300 mL) and washed with water (2×100 mL). The combined organic layer was dried over anhydrous Na₂SO₄ and filtered. The solvent was removed under reduced pressure and the crude compound was purified by flash silica gel (230-400 mesh) column chromatography (40% EtOAc in pet ether) to afford 6-bromo-3-(2,4-dibenzyloxyphenyl)-7-fluoro-1H-benzimidazol-2-one (5, 1 g, 1.71 mmol, 69% yield) LCMS (ES+): m/z 520 [M+H]⁺

### Step 4: 5-bromo-1-(2,6-dibenzyloxy-3-pyridyl)-4-fluoro-3-methyl-benzimidazol-2-one (6)

Into a 100 mL single neck round bottom flask containing a well-stirred solution of 6-bromo-3-(2,6-dibenzyloxy-3-pyridyl)-7-fluoro-1H-benzimidazol-2-one (5, 900 mg, 1.54 mmol) in DMF (20 mL) was added sodium hydride, 60% dispersion in mineral oil (88.40 mg, 2.31 mmol, 60% purity) at 0° C. The reaction mixture was stirred at 25° C. for 30 min. After that, the reaction mixture was cooled to 0° C. and added iodomethane (436.65 mg, 3.08 mmol, 191.51 uL). The reaction mixture was stirred at 25° C. for 2 h. The reaction mixture was quenched with saturated ammonium chloride solution (20 mL) and the solution was extracted with ethyl acetate (2×150 mL). The combined organic layer was dried over Na₂SO₄, filtered and the solvent was removed under vacuum to obtain crude compound, which was purified by flash silica gel column chromatography (40% ethyl acetate in pet ether) to afford 5-bromo-1-(2,6-dibenzyloxy-3-pyridyl)-4-fluoro-3-methyl-benzimidazol-2-one (6, 700 mg, 1.14 mmol, 74% yield) as an off-white solid.

LCMS (ES+): m/z 536.0 [M+H]⁺

### Step 5: tert-butyl 2-[1-[1-(2,6-dibenzyloxy-3-pyridyl)-4-fluoro-3-methyl-2-oxo-benzimidazol-5-yl]-4-piperidyl]acetate (8)

Into a 25 mL pressure tube containing a well-stirred solution of 5-bromo-1-(2,6-dibenzyloxy-3-pyridyl)-4-fluoro-3-methyl-benzimidazol-2-one (6, 150 mg, 243.65 μmol) and tert-butyl 2-(4-piperidyl)acetate (7, 72.83 mg, 365.47 μmol) in anhydrous 1,4-dioxane (8 mL) was added cesium carbonate (238.16 mg, 730.95 μmol) at room temperature. The reaction mixture was degassed by bubbling nitrogen gas for 10 min. Then, Cphos pd G3 (19.65 mg, 24.36 μmol) was added and the mixture was stirred at 100° C. for 16 h. The reaction mixture was filtered through Celite and washed with ethyl acetate (100 mL). The filtrate was concentrated under reduced pressure and the crude compound was purified by flash silica gel column chromatography (30% ethyl acetate in pet ether) to afford tert-butyl 2-[1-[1-(2,6-dibenzyloxy-3-pyridyl)-4-fluoro-3-methyl-2-oxo-benzimidazol-5-yl]-4-piperidyl]acetate (8, 70 mg, 96.13 μmol, 39% yield) as a colorless gummy solid.

LCMS (ES+): m/z 653.2 [M+H]⁺

### Step 6: tert-butyl 2-[1-[1-(2,6-dioxo-3-piperidyl)-4-fluoro-3-methyl-2-oxo-benzimidazol-5-yl]-4-piperidyl]acetate (9)

Into a 25 mL single-neck round-bottom flask containing a well-stirred solution of tert-butyl 2-[1-[1-(2,6-dibenzyloxy-3-pyridyl)-4-fluoro-3-methyl-2-oxo-benzimidazol-5-yl]-4-piperidyl]acetate (8, 250 mg, 361.12 μmol) in anhydrous 1,4-dioxane (6 mL) was added palladium hydroxide on carbon (250 mg, 356.04 μmol, 20% purity) at room temperature. The suspension was stirred under hydrogen bladder pressure at room temperature for 16 h. The reaction mixture was filtered through Celite and washed with 1,4-dioxane (200 mL). The filtrate was concentrated under reduced pressure to get crude tert-butyl 2-[1-[1-(2,6-dioxo-3-piperidyl)-4-fluoro-3-methyl-2-oxo-benzimidazol-5-yl]-4-piperidyl]acetate (9, 190 mg, 350.15 μmol, 97% yield) as a colorless solid.

LCMS (ES+): m/z 475.2 [M+H]⁺

### Step 7: 2-[1-[1-(2,6-dioxo-3-piperidyl)-4-fluoro-3-methyl-2-oxo-benzimidazol-5-yl]-4-piperidyl]acetic acid (10)

Into a 50 mL single-neck round-bottom flask containing a well-stirred solution of tert-butyl 2-[1-[1-(2,6-dioxo-3-piperidyl)-4-fluoro-3-methyl-2-oxo-benzimidazol-5-yl]-4-piperidyl]acetate (9, 219.46 mg, 404.45 μmol) in anhydrous DCM (5 mL) was added trifluoroacetic acid (2.96 g, 25.96 mmol) at room temperature. The solution was stirred at room temperature for 3 h. The reaction mixture was concentrated to dryness to get crude compound 2-[1-[1-(2,6-dioxo-3-piperidyl)-4-fluoro-3-methyl-2-oxo-benzimidazol-5-yl]-4-piperidyl]acetic acid (10, 210 mg, 295.61 μmol, 73% yield, TFA salt) as light brown gummy solid.

LCMS (ES+): m/z 419.2 [M+H]⁺

### 2-(4-(1-(2,6-bis(benzyloxy)pyridin-3-yl)-3-ethyl-2-oxo-2,3-dihydro-1H-benzo[d]imidazol-5-yl)-3-fluorophenyl)acetic acid (5)

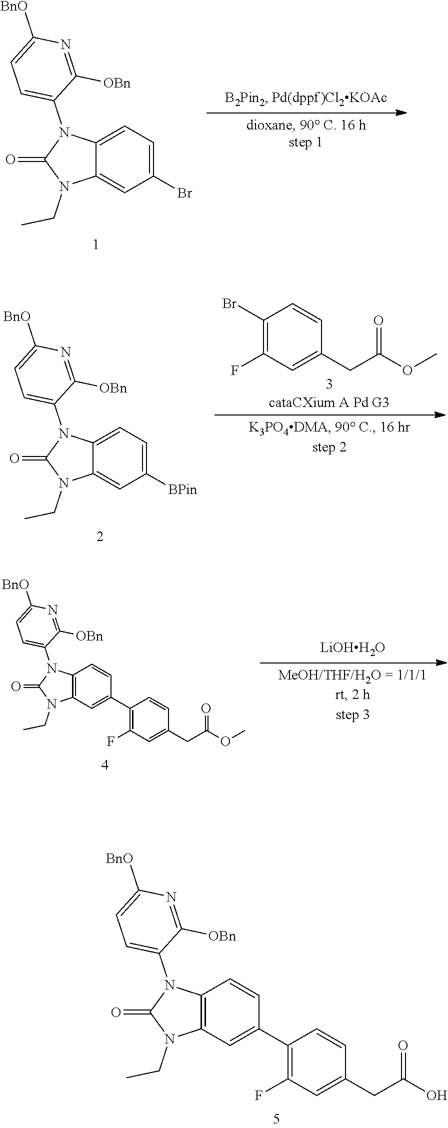

### Step 1:1-(2,6-bis(benzyloxy)pyridin-3-yl)-3-ethyl-5-(4,4,5,5-tetramethyl-1,3,2-dioxaborolan-2-yl)-1H-benzo[d]imidazol-2 (3H)-one (2)

To a solution of 1-(2,6-bis(benzyloxy)pyridin-3-yl)-5-bromo-3-ethyl-11H-benzo[d]imidazol-2 (3H)-one (1, 0.5 g, 942.66 μmol, 1 eq) and bis(pinacol)diborane (359.07 mg, 1.41 mmol, 1.5 eq) in 1,4-dioxane (5 mL) were added KOAc (462.58 mg, 4.71 mmol, 5 eq) and Pd(dppf)Cl₂ (38.49 mg, 47.13 μmol, 0.05 eq) under N₂. The mixture was stirred at 90° C. for 16 h. The reaction mixture was filtered and the filtrate was concentrated under vacuum to give a residue. The residue was purified by flash silica gel chromatography (flow: 36 mL/min; gradient of 0-40% ethyl acetate/petroleum ether; ISCO®; 10 g Sepa Flash® Silica Flash Column; ethyl acetate/petroleum ether=3/1) to afford 1-(2,6-bis(benzyloxy)pyridin-3-yl)-3-ethyl-5-(4,4,5,5-tetramethyl-1,3,2-dioxaborolan-2-yl)-1H-benzo[d]imidazol-2 (3H)-one (2, 500 mg, 787.91 μmol, 83% yield) as a yellow solid.

LCMS (ESI): m/z 578.3 [M+H]⁺

¹H NMR (400 MHz, CDCl3) δ 7.62 (d, J=8.0 Hz, 1H), 7.54-7.50 (m, 1H), 7.48 (s, 1H), 7.46-7.41 (m, 2H), 7.41-7.32 (m, 3H), 7.25 (s, 5H), 6.73 (d, J=8.0 Hz, 1H), 6.52 (d, J=8.0 Hz, 1H), 5.47-5.26 (m, 4H), 4.03 (q, J=7.2 Hz, 2H), 1.38 (s, 12H), 1.25 (t, J=7.2 Hz, 3H).

### Step 2: methyl 2-(4-(1-(2,6-bis(benzyloxy)pyridin-3-yl)-3-ethyl-2-oxo-2,3-dihydro-1H-benzo[d]imidazol-5-yl)-3-fluorophenyl)acetate (4)

Into at 40 mL sealed tube reactor containing a well-stirred solution of 1-(2,6-bis(benzyloxy)pyridin-3-yl)-3-ethyl-5-(4,4,5,5-tetramethyl-1,3,2-dioxaborolan-2-yl)-1H-benzo[d]imidazol-2 (3H)-one (2, 450 mg, 779.25 μmol, 1 eq) and methyl 2-(4-bromo-3-fluorophenyl)acetate (3, 231.03 mg, 935.10 μmol, 1.2 eq) in anhydrous DMA (20 mL) was added K₃PO₄ (496.23 mg, 2.34 mmol, 3 eq) at 20° C. under nitrogen atmosphere and followed by cataCXium A Pd G3 (56.83 mg, 77.93 μmol, 0.1 eq). The resulting mixture was degassed by bubbling nitrogen gas into the reaction mixture for 5 min and heated to 90° C. for 16 h. The reaction mixture was concentrated under vacuum to give a residue. The residue was purified by flash silica gel chromatography (flow: 40 mL/min; gradient of 0-100% ethyl acetate/petroleum ether; ISCO®; 10 g SepaFlash® Silica Flash Column; ethyl acetate/petroleum ether=1/1) to afford methyl 2-(4-(1-(2,6-bis(benzyloxy)pyridin-3-yl)-3-ethyl-2-oxo-2,3-dihydro-1H-benzo[d]imidazol-5-yl)-3-fluorophenyl)acetate (4, 440 mg, 705.24 μmol, 90% yield) as a yellow solid.

LCMS (ESI): m/z 618.2 [M+H]⁺.

### Step 3: 2-(4-(1-(2,6-bis(benzyloxy)pyridin-3-yl)-3-ethyl-2-oxo-2,3-dihydro-1H-benzo[d]imidazol-5-yl)-3-fluorophenyl)acetic acid (5)

To a solution of methyl 2-(4-(1-(2,6-bis(benzyloxy)pyridin-3-yl)-3-ethyl-2-oxo-2,3-dihydro-1H-benzo[d]imidazol-5-yl)-3-fluorophenyl)acetate (4, 440 mg, 712.36 μmol, 1 eq) in H₂O (2 mL), THF (2 mL) and MeOH (2 mL) was added LiOH H₂O (149.47 mg, 3.56 mmol, 5 eq) at 25° C. and the mixture was stirred at 25° C. for 2 h. The reaction mixture was adjusted pH to 6 by 1N HCl aqueous. The mixture was diluted with H₂O (40 mL) and extracted with ethyl acetate (50 mL×2). The combined organic layers were washed with brine (60 mL), dried over Na₂SO₄, filtered and concentrated under vacuum to give a residue. The residue was purified by column chromatography (SiO₂, petroleum ether/ethyl acetate=4/1 to 0/1 and DCM/MeOH=10/1; petroleum ether/ethyl acetate=0/1) to afford 2-(4-(1-(2,6-bis(benzyloxy)pyridin-3-yl)-3-ethyl-2-oxo-2,3-dihydro-1H-benzo[d]imidazol-5-yl)-3-fluorophenyl)acetic acid (5, 320 mg, 524.82 μmol, 74% yield) as yellow solid.

LCMS (ESI): m/z 604.5 [M+H]⁺.

### 2-(4-((3-(2,6-bis(benzyloxy)pyridin-3-yl)-1-methyl-1H-indazol-6-yl)amino)phenyl)acetic acid (4)

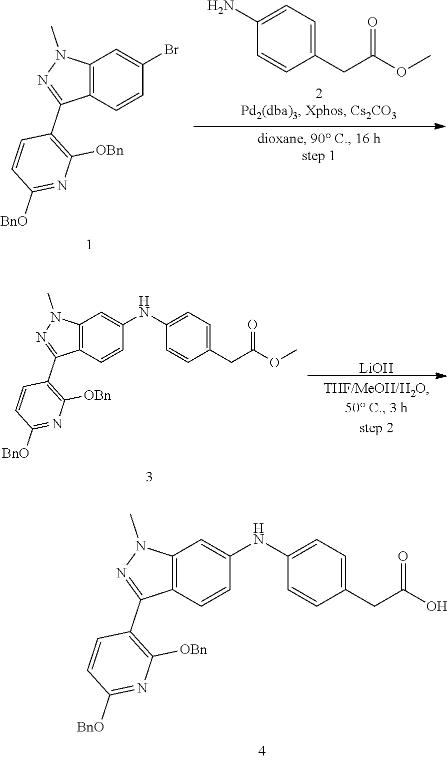 [SMILES: Cn1nc(-c2ccc(OCc3ccccc3)nc2OCc2ccccc2)c2ccc(Br)cc21 ; COC(=O)Cc1ccc(N)cc1 ; COC(=O)Cc1ccc(Nc2ccc3c(-c4ccc(OCc5ccccc5)nc4OCc4ccccc4)nn(C)c3c2)cc1 ; "LiOH" ; Cn1nc(-c2ccc(OCc3ccccc3)nc2OCc2ccccc2)c2ccc(Nc3ccc(CC(=O)O)cc3)cc21]

### Step 1: methyl 2-(4-((3-(2,6-bis(benzyloxy)pyridin-3-yl)-1-methyl-1H-indazol-6-yl)amino)phenyl)acetate (3)

To a mixture of 3-(2,6-bis(benzyloxy)pyridin-3-yl)-6-bromo-1-methyl-1H-indazole (1, 600 mg, 1.20 mmol, 1 eq), methyl 2-(4-aminophenyl)acetate (2, 198.07 mg, 1.20 mmol, 1 eq), Xphos (114.32 mg, 239.82 μmol, 0.2 eq) and Cs₂CO₃ (781.36 mg, 2.40 mmol, 2 eq) in dioxane (10 mL) was added Pd₂(dba)₃ (109.80 mg, 119.91 μmol, 0.1 eq) under N₂. The mixture was stirred at 90° C. for 16 h under N₂ atmosphere. The mixture was filtered and concentrated in vacuum. The residue was purified by column chromatography (SiO₂, Petroleum ether/Ethyl acetate=100/1 to 1/1) to afford methyl 2-(4-((3-(2,6-bis(benzyloxy)pyridin-3-yl)-1-methyl-1H-indazol-6-yl)amino)phenyl)acetate (3, 540 mg, 923.61 μmol, 77% yield) as yellow solid.

LCMS (ESI): m/z 585.1 [M+H]⁺

### Step 2: 2-(4-((3-(2,6-bis(benzyloxy)pyridin-3-yl)-1-methyl-1H-indazol-6-yl)amino)phenyl)acetic acid (4)

A mixture of methyl 2-(4-((3-(2,6-bis(benzyloxy)pyridin-3-yl)-1-methyl-1H-indazol-6-yl)amino)phenyl)acetate (3, 540 mg, 923.61 μmol, 1 eq) and LiOH H₂O (387.58 mg, 9.24 mmol, eg) in H₂O (6 mL), MeOH (6 mL) and THF (6 mL) was stirred at 50° C. for 3 h. The mixture was adjusted to pH-5 by 2 N HCl. The mixture was extracted with EtOAc (100 mL). The organic phase was washed with brine (300 mL), dried with anhydrous Na₂SO₄, filtered and concentrated in vacuum. The residue was purified by column chromatography (SiO₂, Petroleum ether/Ethyl acetate=100/1 to 1/1) to afford 2-(4-((3-(2,6-bis(benzyloxy)pyridin-3-yl)-1-methyl-1H-indazol-6-yl)amino)phenyl)acetic acid (4, 500 mg, 876.22 μmol, 95% yield) as yellow solid.

LCMS (ESI): m/z 571.3 [M+H]⁺

### 2-(4-(1-(2,6-bis(benzyloxy)pyridin-3-yl)-3-ethyl-2-oxo-2,3-dihydro-1H-benzo[d]imidazol-5-yl)phenyl)acetic acid (4)

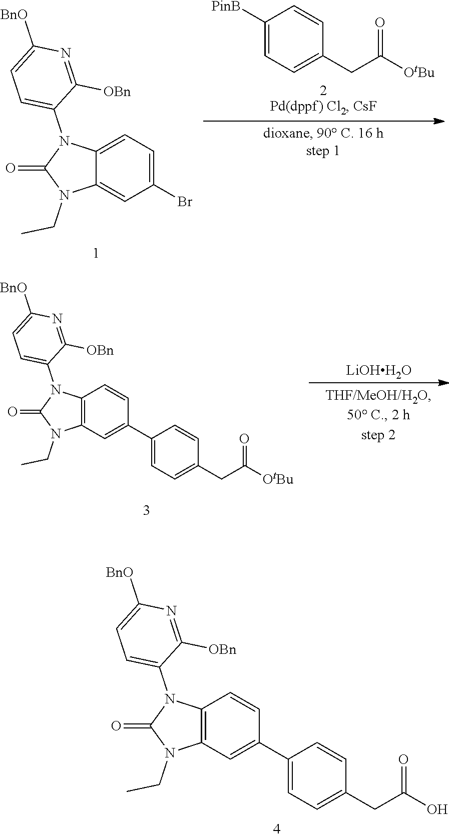

### Step 1: tert-butyl 2-(4-(1-(2,6-bis(benzyloxy)pyridin-3-yl)-3-ethyl-2-oxo-2,3-dihydro-1H-benzo[d]imidazol-5-yl)phenyl)acetate (3)

A mixture of 1-(2,6-bis(benzyloxy)pyridin-3-yl)-5-bromo-3-ethyl-1H-benzo[d]imidazol-2 (3H)-one (1, 440.96 mg, 1.39 mmol, 1 eq), tert-butyl 2-(4-(4,4,5,5-tetramethyl-1,3,2-dioxaborolan-2-yl)phenyl)acetate (2, 490 mg, 923.81 μmol, 1.5 eq) and CsF (420.98 mg, 2.77 mmol, 3 eq) in dioxane (16 mL) was degassed and purged with N₂ for 3 times, and then Pd(dppf)Cl₂ (67.60 mg, 92.38 μmol, 0.1 eq) was added to the mixture and the mixture was stirred at 90° C. for 12 h under N₂ atmosphere. The reaction mixture was filtered and concentrated under reduced pressure. The residue was purified by column chromatography (SiO₂, PE/EA=1/0 to 1/1) to afford tert-butyl 2-(4-(1-(2,6-bis(benzyloxy)pyridin-3-yl)-3-ethyl-2-oxo-2,3-dihydro-1H-benzo[d]imidazol-5-yl)phenyl)acetate (3, 540 mg, 732.06 μmol, 79% yield) as a yellow solid.

LCMS (ESI): m/z 642.3 [M+H]⁺

### Step 2: 2-(4-(1-(2,6-bis(benzyloxy)pyridin-3-yl)-3-ethyl-2-oxo-2,3-dihydro-1H-benzo[d]imidazol-5-yl)phenyl)acetic acid (4)

To a solution of tert-butyl 2-(4-(1-(2,6-bis(benzyloxy)pyridin-3-yl)-3-ethyl-2-oxo-2,3-dihydro-1H-benzo[d]imidazol-5-yl)phenyl)acetate (3, 540 mg, 841.44 μmol, 1 eq) in MeOH (2 mL), H₂O (2 mL) and THF (2 mL) was added LiOH H₂O (353.10 mg, 8.41 mmol, 233.84 μL, 10 eq). The mixture was stirred at 50° C. for 2 h. The reaction mixture was concentrated under reduced pressure. The residue was purified by column chromatography (SiO₂, PE/EA=1/0 to 1/1) to afford 2-(4-(1-(2,6-bis(benzyloxy)pyridin-3-yl)-3-ethyl-2-oxo-2,3-dihydro-1H-benzo[d]imidazol-5-yl)phenyl)acetic acid (4, 230 mg, 388.80 μmol, 46% yield) as yellow oil.

LCMS (ESI): m/z 586.4 [M+H]⁺

### 2-[3-[1-(2,6-dioxo-3-piperidyl)-3-methyl-2-oxo-benzimidazol-5-yl]-8-azabicyclo[3.2.1]octan-8-yl]acetic acid (3)

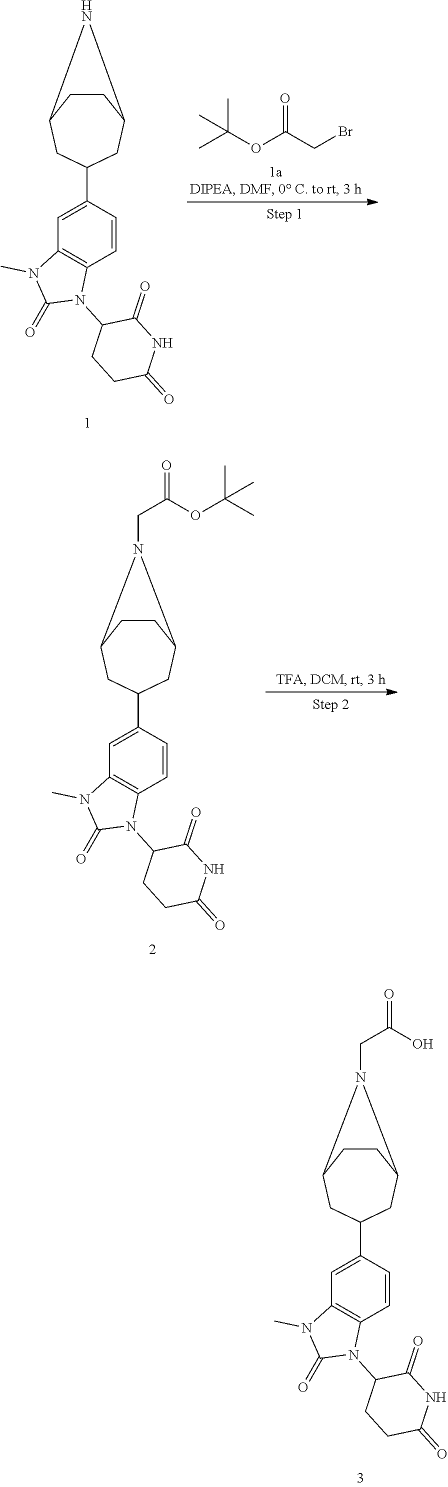

### Step 1: tert-butyl 2-[3-[1-(2,6-dioxo-3-piperidyl)-3-methyl-2-oxo-benzimidazol-5-yl]-8-azabicyclo[3.2.1]octan-8-yl]acetate (2)

Into a 50 mL round-bottom flask containing a well-stirred solution of 3-[5-(8-azabicyclo[3.2.1]octan-3-yl)-3-methyl-2-oxo-benzimidazol-1-yl]piperidine-2,6-dione (1, 220 mg, 456.00 μmol, TFA salt) in DMF (5 mL) was added DIPEA (385.87 mg, 2.99 mmol, 520.05 uL), the suspension was cooled to 0° C. and added tert-butylbromoacetate (1a, 151.41 mg, 776.27 μmol). The reaction mixture was stirred at ambient temperature for 3 h. The reaction mixture was quenched with water (15 mL) and The precipitate was filtered, washed with water (15 mL) and dried to afford tert-butyl 2-[3-[1-(2,6-dioxo-3-piperidyl)-3-methyl-2-oxo-benzimidazol-5-yl]-8-azabicyclo[3.2.1]octan-8-yl]acetate (2, 300 mg, 410.92 μmol, 90% yield) as a pink solid.

LCMS (ES+): m/z 483.2 [M+H]⁺

### Step 2: 2-[3-[1-(2,6-dioxo-3-piperidyl)-3-methyl-2-oxo-benzimidazol-5-yl]-8-azabicyclo[3.2.1]octan-8-yl]acetic acid (3)

Into a 25 mL round-bottom flask containing a well-stirred solution of tert-butyl 2-[3-[1-(2,6-dioxo-3-piperidyl)-3-methyl-2-oxo-benzimidazol-5-yl]-8-azabicyclo[3.2.1]octan-8-yl]acetate (2, 300 mg, 410.92 μmol) in DCM (5 mL) was added TFA (468.55 mg, 4.11 mmol) dropwise at 0° C. The reaction mixture was stirred at room temperature for 1 h. The volatiles were distilled off under reduced pressure and the residue was triturated with MTBE (20 mL), filtered and dried to get 2-[3-[1-(2,6-dioxo-3-piperidyl)-3-methyl-2-oxo-benzimidazol-5-yl]-8-azabicyclo[3.2.1]octan-8-yl]acetic acid (3, 220 mg, 305.16 μmol, 74% yield, TFA salt) as a brown solid.

LCMS (ES+): m/z 427.2 [M+H]⁺

### 3-(5-(1-(2-aminoethyl)piperidin-4-yl)-3-methyl-2-oxo-2,3-dihydro-1H-benzo[d]imidazol-1-yl)piperidine-2,6-dione (8)

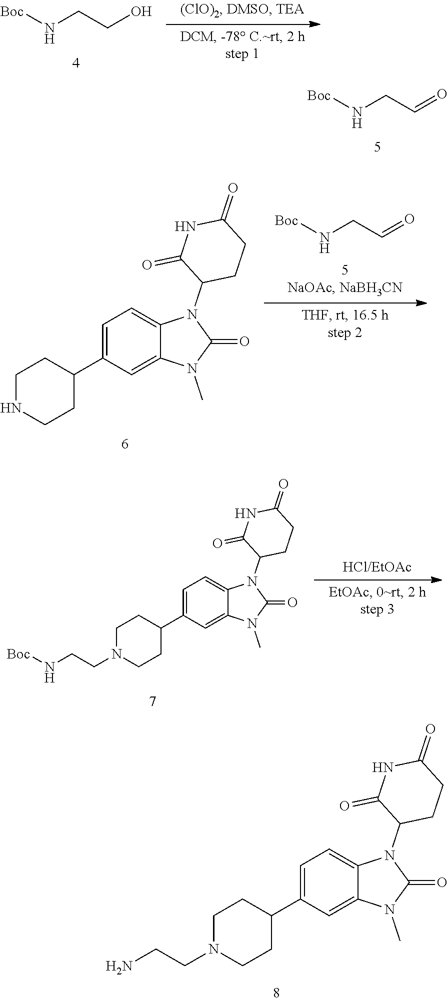 [SMILES: CC(C)(C)OC(=O)NCCO ; CC(C)(C)OC(=O)NCC=O ; Cn1c(=O)n(C2CCC(=O)NC2=O)c2ccc(C3CCNCC3)cc21 ; CC(C)(C)OC(=O)NCC=O ; Cn1c(=O)n(C2CCC(=O)NC2=O)c2ccc(C3CCN(CCNC(=O)OC(C)(C)C)CC3)cc21 ; Cn1c(=O)n(C2CCC(=O)NC2=O)c2ccc(C3CCN(CCN)CC3)cc21]

### Step 1: tert-butyl (2-oxoethyl)carbamate (5)

To a solution of DMSO (6.06 g, 77.54 mmol, 5.51 mL, 2.5 eq) in DCM (30 mL) was added dropwise oxalyl dichloride (7.87 g, 62.04 mmol, 5.39 mL, 2 eq) at −78° C. and stirred −78° C. for 15 mins. To the mixture was added dropwise tert-butyl (2-hydroxyethyl)carbamate (4, 5 g, 31.02 mmol, 4.81 mL, 1 eq) in DCM (30 mL) and stirred at −78° C. for 45 mins. Then TEA (15.69 g, 155.09 mmol, 21.62 mL, 5 eq) was added dropwise at −78° C. The resulting mixture was warmed to 20° C. and stirred at 20° C. for 1 h. The reaction mixture was diluted with water (80 mL) and extracted with dichloromethane (50 mL×3). The combined organic layers were washed with brine (100 mL), dried over Na₂SO₄, filtered and concentrated under reduced pressure to give a residue. The residue was purified by flash silica gel chromatography (60 mL/min, Eluent of 0-100% ethyl acetate/petroleum ether gradient, Column: ISCO; 40 g SepaFlash Silica Flash Column; petroleum ether/ethyl acetate=1/1) to afford tert-butyl (2-oxoethyl)carbamate (5, 1.3 g, 8.17 mmol, 26% yield) as yellow oil.

¹H NMR (400 MHz, d₆-DMSO) δ 9.46 (s, 1H), 7.21 (s, 1H), 3.74 (d, J=5.6 Hz, 2H), 1.40 (s, 9H).

### Step 2: tert-butyl (2-(4-(1-(2,6-dioxopiperidin-3-yl)-3-methyl-2-oxo-2,3-dihydro-1H-benzo[d]imidazol-5-yl)piperidin-1-yl)ethyl)carbamate (7)

To a solution of 3-(3-methyl-2-oxo-5-(piperidin-4-yl)-2,3-dihydro-1H-benzo[d]imidazol-1-yl)piperidine-2,6-dione (6, 400 mg, 1.17 mmol, 1 eq) and tert-butyl (2-oxoethyl)carbamate (5, 371.93 mg, 2.34 mmol, 1 eq) in THF (20 mL) was added NaOAc (191.67 mg, 2.34 mmol, 125.28 μL, 2 eq), Then the mixture was stirred at 20° C. for 0.5 h. To the mixture was added NaBH₃CN (367.08 mg, 5.84 mmol, 5 eq) and stirred at 20° C. for 16 h. The reaction mixture was concentrated under reduced pressure to give residue. The residue was purified by flash silica gel chromatography (60 mL/min, Eluent of 0-100% ethyl acetate/petroleum ether and 0-20% dichloromethane/methanol, Column: ISCO; 5 g SepaFlash Silica Flash Column; DCM/MeOH=10/1) to afford tert-butyl (2-(4-(1-(2,6-dioxopiperidin-3-yl)-3-methyl-2-oxo-2,3-dihydro-1H-benzo[d]imidazol-5-yl)piperidin-1-yl)ethyl)carbamate (7, 220 mg, 412.30 μmol, 35% yield) as a white solid.

LCMS (ESI): m/z 486.2 [M+H]⁺

### Step 3: 3-(5-(1-(2-aminoethyl)piperidin-4-yl)-3-methyl-2-oxo-2,3-dihydro-1H-benzo[d]imidazol-1-yl)piperidine-2,6-dione (8)

To a solution of tert-butyl (2-(4-(1-(2,6-dioxopiperidin-3-yl)-3-methyl-2-oxo-2,3-dihydro-1H-benzo[d]imidazol-5-yl)piperidin-1-yl)ethyl)carbamate (7, 220 mg, 453.07 μmol, 1 eq) in EtOAc (5 mL) was added HCl/EtOAc (4 M, 5 mL, 44 eq) at 0° C. The mixture was stirred at 20° C. for 2 h. The reaction mixture was concentrated under reduced pressure to afford 3-(5-(1-(2-aminoethyl)piperidin-4-yl)-3-methyl-2-oxo-2,3-dihydro-1H-benzo[d]imidazol-1-yl)piperidine-2,6-dione (8, 200 mg, 518.86 μmol, 99% yield) as a white solid. The crude product was used in the next step without further purification.

LCMS (ESI): m/z 386.5 [M+H]⁺

¹H NMR (400 MHz, d₆-DMSO) δ 11.16-10.88 (m, 2H), 7.14-7.00 (m, 2H), 6.92 (d, J=7.6 Hz, 1H), 5.37 (dd, J=5.2, 12.4 Hz, 1H), 3.73-3.60 (m, 2H), 3.35 (s, 3H), 3.15 (d, J=8.0 Hz, 2H), 2.98-2.85 (m, 2H), 2.77-2.54 (m, 3H), 2.24-1.93 (m, 6H), 1.91 (s, 2H).

### 4-(((1-(2,6-dioxopiperidin-3-yl)-2-oxo-1,2-dihydrobenzo[cd]indol-6-yl)methyl)amino)-4-oxobutanoic acid (2)

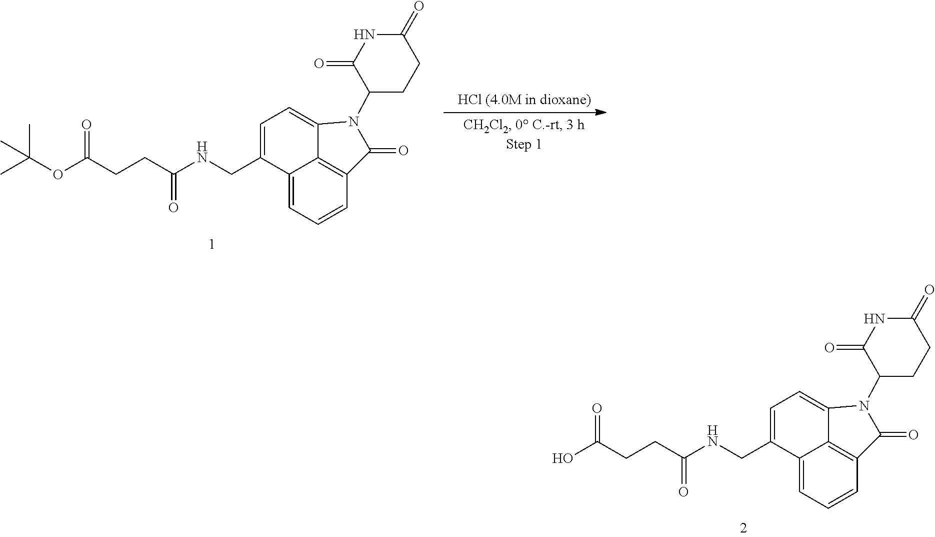 [SMILES: CC(C)(C)OC(=O)CCC(=O)NCc1ccc2c3c(cccc13)C(=O)N2C1CCC(=O)NC1=O ; O=C(O)CCC(=O)NCc1ccc2c3c(cccc13)C(=O)N2C1CCC(=O)NC1=O]

### Step 1: 4-(((1-(2,6-dioxopiperidin-3-yl)-2-oxo-1,2-dihydrobenzo[cd]indol-6-yl)methyl)amino)-4-oxobutanoic acid (2)

Into a 100 mL single-neck round-bottom flask containing a well-stirred solution of tert-butyl 4-[[1-(2,6-dioxo-3-piperidyl)-2-oxo-benzo[cd]indol-6-yl]methylamino]-4-oxo-butanoate (700 mg, 1.35 mmol) in anhydrous DCM (10 mL) at 0° C. was added HCl (4.0 M in dioxane, 33.83 mmol, 8.5 mL) dropwise. The reaction mixture was stirred at room temperature for 3 h. The reaction mixture was concentrated under reduced pressure and triturated with MTBE to obtain 4-(((1-(2,6-dioxopiperidin-3-yl)-2-oxo-1,2-dihydrobenzo[cd]indol-6-yl)methyl)amino)-4-oxobutanoic acid (2, 555 mg, 1.20 mmol, 89% yield) as a yellow solid.

LCMS (ES−): m/z 408.0 [M−H]⁻

### 1-(2,6-bis(benzyloxy)pyridin-3-yl)-6-fluoro-3-methyl-5-(piperazin-1-yl)-1H-benzo[d]imidazol-2 (3H)-one (9)

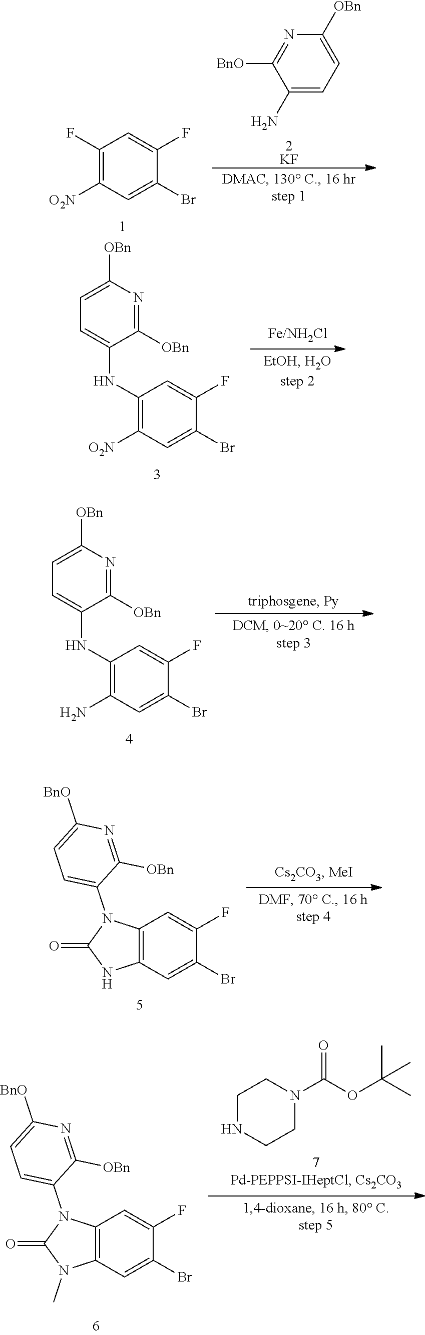

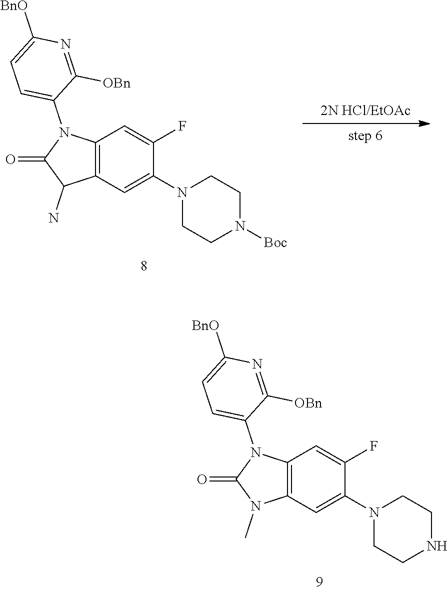 [SMILES: CC(C)(C)OC(=O)N1CCN(c2cc3c(cc2F)N(c2ccc(OCc4ccccc4)nc2OCc2ccccc2)C(=O)C3N)CC1 ; Cn1c(=O)n(-c2ccc(OCc3ccccc3)nc2OCc2ccccc2)c2cc(F)c(N3CCNCC3)cc21]

### Step 1: 2,6-bis(benzyloxy)-N-(4-bromo-5-fluoro-2-nitrophenyl)pyridin-3-amine (3)

To a solution of 1-bromo-2,4-difluoro-5-nitrobenzene (1, 1.32 g, 5.55 mmol, 1 eq) and 2,6-bis(benzyloxy)pyridin-3-amine (2, 1.7 g, 5.55 mmol, 1 eq) in DMAC (1 mL) was added KF (386.86 mg, 6.66 mmol, 1.2 eq). The mixture was stirred at 130° C. for 16 h. The reaction mixture was combined with another three batches (each 100 mg with NMP/DMAC/DMSO as solvent). The mixture was diluted with water (100 mL) and extracted with ethyl acetate (100 mL×2). The combined organic layers were washed with brine (100 mL×2), dried over Na₂SO₄, filtered and concentrated under reduced pressure to afford 2,6-bis(benzyloxy)-N-(4-bromo-5-fluoro-2-nitrophenyl)pyridin-3-amine (3, 3.8 g, 5.51 mmol, 99% yield) as a brown solid. The crude product was used in the next step without further purification.

LCMS (ESI): m/z 525.8 [M+H]⁺

### Step 2: N¹-(2,6-bis(benzyloxy)pyridin-3-yl)-4-bromo-5-fluorobenzene-1,2-diamine (4)

To a mixture of 2,6-bis(benzyloxy)-N-(4-bromo-5-fluoro-2-nitrophenyl)pyridin-3-amine (3, 3.8 g, 7.25 mmol, leg) in water (40 mL) and EtOH (100 mL) were added Fe (2.02 g, 36.24 mmol, 5 eq) and NH₄Cl (1.94 g, 36.24 mmol, 5 eq). The mixture was stirred at 80° C. for 16 h. The mixture was filtered and washed with EtOH (50 ml×3). The filtrate was concentrated to give a residue. The residue was diluted with ethyl acetate (200 mL), washed with brine (100 mL×2), dried over Na₂SO₄, filtered and concentrated under reduced pressure to give a residue. The residue was purified by column chromatography (SiO₂, petroleum ether/ethyl acetate=0/1 to 3/1; petroleum ether/ethyl acetate=3/1) to afford N¹-(2,6-bis(benzyloxy)pyridin-3-yl)-4-bromo-5-fluorobenzene-1,2-diamine (4, 2 g, 3.20 mmol, 44% yield) as a yellow solid.

LCMS (ESI): m/z 496.2 [M+H]+

### Step 3: 1-(2,6-bis(benzyloxy)pyridin-3-yl)-5-bromo-6-fluoro-1H-benzo[d]imidazol-2 (3H)-one (5)

To a solution of N¹-(2,6-bis(benzyloxy)pyridin-3-yl)-4-bromo-5-fluorobenzene-1,2-diamine (4, 2.2 g, 4.45 mmol, 1 eq) and pyridine (3.52 g, 44.50 mmol, 3.60 mL, 10 eq) in DCM (40 mL) was added a solution of triphosgene (2.64 g, 8.90 mmol, 2 eq) in DCM (40 mL) at 0-10° C. The mixture was stirred at 30° C. for 16 h. The mixture was poured into ice-water (100 mL) and extracted with DCM (100 mL×2). The organic phases were combined and washed with brine (50 mL×2), dried over anhydrous Na₂SO₄, filtered and the filtrate was concentrated under reduced pressure to give a residue. The residue was purified by column chromatography (SiO₂, petroleum ether/ethyl acetate=1/0 to 2/1: petroleum ether/ethyl acetate=3/1) to afford 1-(2,6-bis(benzyloxy)pyridin-3-yl)-5-bromo-6-fluoro-1H-benzo[d]imidazol-2 (3H)-one (5, 1.7 g, 3.17 mmol, 71% yield) as a white solid.

LCMS (ESI): m/z 521.9 [M+H]⁺

### Step 4: 1-(2,6-bis(benzyloxy)pyridin-3-yl)-5-bromo-6-fluoro-3-methyl-1H-benzo[d]imidazol-2 (3H)-one (6)

To a mixture of 1-(2,6-bis(benzyloxy)pyridin-3-yl)-5-bromo-6-fluoro-1H-benzo[d]imidazol-2 (3H)-one (5, 1.7 g, 3.27 mmol, 1 eq) and Cs₂CO₃ (2.13 g, 6.53 mmol, 2 eq) in DMF (20 mL) was added Mel (927.44 mg, 6.53 mmol, 406.77 μL, 2 eq). The mixture was stirred at 70° C. for 16 h. The mixture was combined with another 110 mg batch. The mixture was diluted with water (100 mL) and extracted with ethyl acetate (50 mL×2). The combined organic layers were washed with brine (50 mL×2), dried over Na₂SO₄, filtered and concentrated under reduced pressure to afford 1-(2,6-bis(benzyloxy)pyridin-3-yl)-5-bromo-6-fluoro-3-methyl-1H-benzo[d]imidazol-2 (3H)-one (6, 1.7 g, 3.09 mmol, 94% yield) as a yellow solid. The crude product was used in the next step without further purification.

LCMS (ESI): m/z 536.1 [M+H]⁺

### Step 5: tert-butyl 4-(1-(2,6-bis(benzyloxy)pyridin-3-yl)-6-fluoro-3-methyl-2-oxo-2,3-dihydro-1H-benzo[d]imidazol-5-yl)piperazine-1-carboxylate (8)

To a mixture of 1-(2,6-bis(benzyloxy)pyridin-3-yl)-5-bromo-6-fluoro-3-methyl-1H-benzo[d]imidazol-2 (3H)-one (6, 500 mg, 935.67 μmol, 1 eq), tert-butyl piperazine-1-carboxylate (7, 348.54 mg, 1.87 mmol, 2 eq), Cs₂CO₃ (914.58 mg, 2.81 mmol, 3 eq) in dioxane (12 mL) was added Pd-PEPPSI-IHeptCl (45.51 mg, 46.78 μmol, 0.05 eq) under N₂. The mixture was stirred at 90 C for 16 h. The reaction mixture was concentrated under reduced pressure to give a residue. The residue was purified by column chromatography (SiO₂, petroleum ether/ethyl acetate=1/0 to 1/1; petroleum ether/ethyl acetate=1/1) to afford tert-butyl 4-(1-(2,6-bis(benzyloxy)pyridin-3-yl)-6-fluoro-3-methyl-2-oxo-2,3-dihydro-1H-benzo[d]imidazol-5-yl)piperazine-1-carboxylate (8, 500 mg, 765.97 μmol, 82% yield) as yellow oil.

LCMS (ESI): m/z 640.2 [M+H]⁺

¹H NMR (400 MHz, DMSO-d₆) δ 7.77 (d, J=8.0 Hz, 1H), 7.48-7.43 (m, 2H), 7.42-7.31 (m, 3H), 7.27 (s, 5H), 7.01 (d, J=7.2 Hz, 1H), 6.64-6.57 (m, 2H), 5.50-5.26 (m, 4H), 3.49-3.46 (s, 4H), 3.36 (s, 3H), 2.97-2.87 (m, 4H), 1.43 (s, 9H).

### Step 6: 1-(2,6-bis(benzyloxy)pyridin-3-yl)-6-fluoro-3-methyl-5-(piperazin-1-yl)-1H-benzo[d]imidazol-2 (3H)-one (9)

To a solution of tert-butyl 4-(1-(2,6-bis(benzyloxy)pyridin-3-yl)-6-fluoro-3-methyl-2-oxo-2,3-dihydro-1H-benzo[d]imidazol-5-yl)piperazine-1-carboxylate (8, 400 mg, 625.28 μmol, 1 eq) in ethyl acetate (4 mL) was added HCl/EtOAc (4 M, 4.00 mL, 26 eq) at 0-10° C. Then the mixture was stirred at 30° C. for 2 h. The reaction mixture was combined with another 50 mg batch and adjusted pH to 7 with sat-NaHCO₃ at 0-10° C. Then the mixture was extracted with DCM (30 mL×2). The combined organic layers were washed with brine (30 mL×2), dried over Na₂SO₄, filtered and concentrated under reduced pressure to afford 1-(2,6-bis(benzyloxy)pyridin-3-yl)-6-fluoro-3-methyl-5-(piperazin-1-yl)-1H-benzo[d]imidazol-2 (3H)-one (9, 300 mg, 283.54 μmol, 45% yield) as a yellow solid. The crude product was used in the next step without further purification.

LCMS (ESI): m/z 540.2 [M+H]⁺

### 2-(4-(1-(2,6-bis(benzyloxy)pyridin-3-yl)-4-fluoro-3-methyl-2-oxo-2,3-dihydro-1H-benzo[d]imidazol-5-yl)-3-fluorophenyl)acetic acid (5)

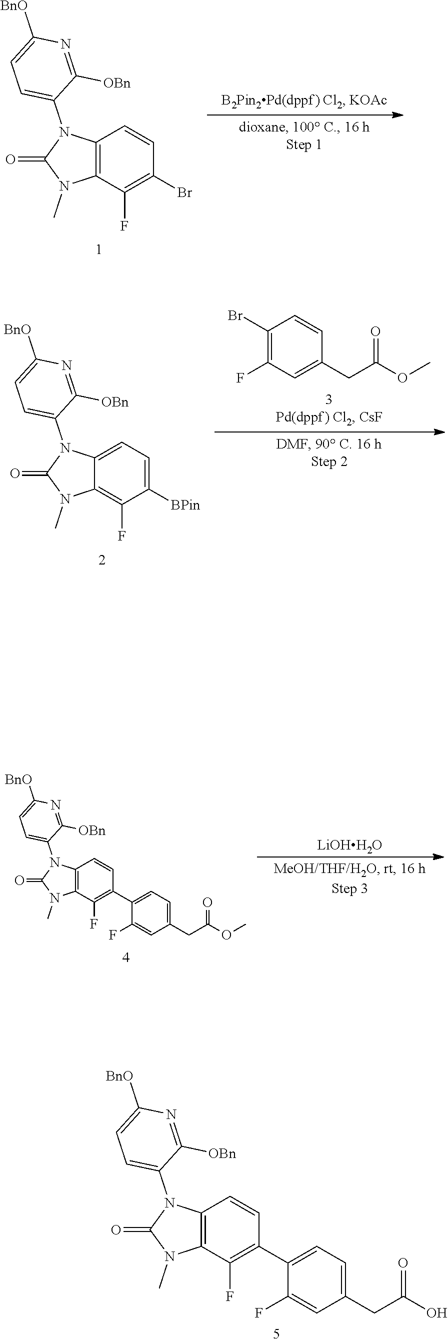

### Step 1:1-(2,6-bis(benzyloxy)pyridin-3-yl)-4-fluoro-3-methyl-5-(4,4,5,5-tetramethyl-1,3,2-dioxaborolan-2-yl)-1H-benzo[d]imidazol-2 (3H)-one (2)

A mixture of 1-(2,6-bis(benzyloxy)pyridin-3-yl)-5-bromo-4-fluoro-3-methyl-1H-benzo[d]imidazol-2 (3H)-one (1, 500 mg, 935.67 μmol, 1 eq), bis(pinacol)diborane (475.20 mg, 1.87 mmol, 2 eq), Pd(dppf)Cl₂ (68.46 mg, 93.57 μmol, 0.1 eq and KOAc (275.49 mg, 2.81 mmol, 3 eq) in dioxane (5 mL) was degassed and purged with N₂ 3 times, and then the mixture was stirred at 100° C. for 16 hr under N₂ atmosphere. The reaction mixture was poured into water (50 mL) and extracted with ethyl acetate (2×50 mL). Organic phases were combined and washed with brine (50 mL), dried by anhydrous Na₂SO₄, filtered and concentrated in vacuum. The residue was purified by flash silica gel chromatography (SiO₂, petroleum ether/ethyl acetate=1/0 to 1/1) to afford 1-(2,6-bis(benzyloxy)pyridin-3-yl)-4-fluoro-3-methyl-5-(4,4,5,5-tetramethyl-1,3,2-dioxaborolan-2-yl)-1H-benzo[d]imidazol-2 (3H)-one (2, 368 mg, 607.59 μmol, 65% yield) as a yellow solid.

LCMS (ESI): m/z 582.1 [M+H]⁺

¹H NMR (400 MHz, d₆-DMSO) δ 7.82 (d, J=8.4 Hz, 1H), 7.45-7.42 (m, 2H), 7.41-7.33 (m, 3H), 7.30-7.23 (m, 6H), 6.60 (dd, J=8.0, 18.4 Hz, 2H), 5.42-5.32 (m, 4H), 3.54 (d, J=1.6 Hz, 3H), 1.30 (s, 12H).

### Step 2: methyl 2-(4-(1-(2,6-bis(benzyloxy)pyridin-3-yl)-4-fluoro-3-methyl-2-oxo-2,3-dihydro-1H-benzo[d]imidazol-5-yl)-3-fluorophenyl)acetate (4)

A mixture of 1-(2,6-bis(benzyloxy)pyridin-3-yl)-4-fluoro-3-methyl-5-(4,4,5,5-tetramethyl-1,3,2-dioxaborolan-2-yl)-1H-benzo[d]imidazol-2 (3H)-one (2, 340 mg, 584.75 μmol, 1 eq), methyl 2-(4-bromo-3-fluorophenyl)acetate (3, 187.81 mg, 760.18 μmol, 1.3 eq, CsF (266.48 mg, 1.75 mmol, 3 eq) and Pd(dppf)Cl₂ (42.79 mg, 58.48 μmol, 0.1 eq) in DMF (3 mL) was degassed and purged with N₂ 3 times, and then the mixture was stirred at 90° C. for 16 hr under N₂ atmosphere. The reaction mixture was poured into water (20 mL) and extracted with DCM (2×20 mL). Organic phases were combined and washed with brine (30 mL), dried by anhydrous Na₂SO₄, filtered and concentrated in vacuum. The residue was purified by flash silica gel chromatography (SiO₂, petroleum ether/ethyl acetate=1/0 to 4/1) to afford methyl 2-(4-(1-(2,6-bis(benzyloxy)pyridin-3-yl)-4-fluoro-3-methyl-2-oxo-2,3-dihydro-1H-benzo[d]imidazol-5-yl)-3-fluorophenyl)acetate (4, 220 mg, 350.37 μmol, 60% yield) as a yellow oil.

LCMS (ESI): m/z 622.2 [M+H]⁺

¹H NMR (400 MHz, CDCl3) δ 7.62 (d, J=8.2 Hz, 1H), 7.46-7.32 (m, 6H), 7.29 (s, 1H), 7.26 (s, 4H), 7.18-7.13 (m, 2H), 6.97-6.91 (m, 1H), 6.54 (dd, J=3.6, 8.0 Hz, 2H), 5.52-5.46 (m, 1H), 5.37 (s, 2H), 5.32-5.28 (m, 1H), 3.75 (s, 3H), 3.69 (s, 5H).

### Step 3: 2-(4-(1-(2,6-bis(benzyloxy)pyridin-3-yl)-4-fluoro-3-methyl-2-oxo-2,3-dihydro-1H-benzo[d]imidazol-5-yl)-3-fluorophenyl)acetic acid (5)

To a solution of methyl 2-(4-(1-(2,6-bis(benzyloxy)pyridin-3-yl)-4-fluoro-3-methyl-2-oxo-2,3-dihydro-1H-benzo[d]imidazol-5-yl)-3-fluorophenyl)acetate (4, 220 mg, 353.91 μmol, 1 eq) in THF (2 mL), MeOH (2 mL) and H₂O (2 mL) was added LiOH H₂O (74.26 mg, 1.77 mmol, 49.18 μL, 5 eq). The mixture was stirred at 20° C. for 16 h. The reaction mixture was poured into water (20 mL) and extracted with DCM (2×20 mL). Organic phases were combined and washed with brine (30 mL), filtered and concentrated in vacuum. The residue was purified by flash silica gel chromatography (SiO₂, petroleum ether/ethyl acetate=10/1 to 1/0 to DCM/MeOH=10/1) to afford 2-(4-(1-(2,6-bis(benzyloxy)pyridin-3-yl)-4-fluoro-3-methyl-2-oxo-2,3-dihydro-1H-benzo[d]imidazol-5-yl)-3-fluorophenyl)acetic acid (5, mg, 293.28 μmol, 83% yield) as a white solid.

LCMS (ESI): m/z 608.4 [M+H]⁺

### 1-(2,6-bis(benzyloxy)pyridin-3-yl)-4-fluoro-3-methyl-5-(piperazin-1-yl)-1H-benzo[d]imidazol-2 (3H)-one (10)

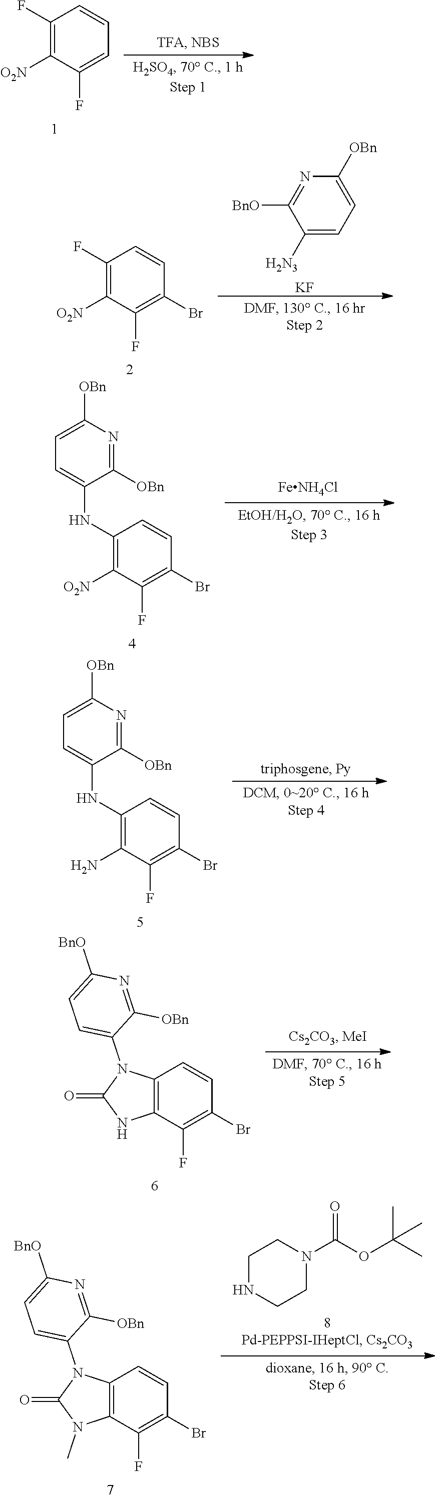

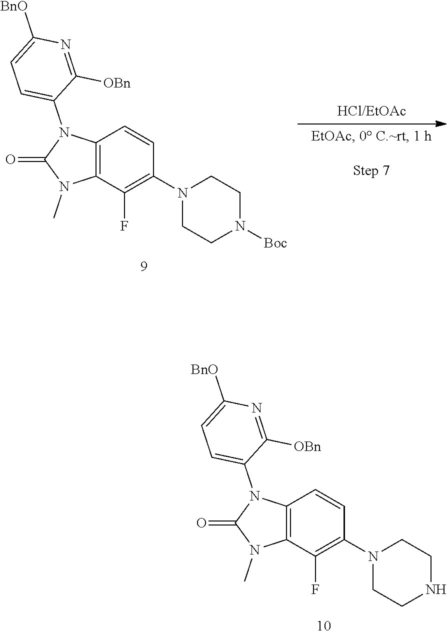 [SMILES: Cn1c(=O)n(-c2ccc(OCc3ccccc3)nc2OCc2ccccc2)c2ccc(N3CCN(C(=O)OC(C)(C)C)CC3)c(F)c21 ; Cn1c(=O)n(-c2ccc(OCc3ccccc3)nc2OCc2ccccc2)c2ccc(N3CCNCC3)c(F)c21]

### Step 1:1-bromo-2,4-difluoro-3-nitrobenzene (3)

A mixture of 1,3-difluoro-2-nitrobenzene (1, 20 g, 125.72 mmol, 13.33 mL, 1 eq) and NBS (22.38 g, 125.72 mmol, 10.65 mL, 1 eq) in TFA (106.56 g, 934.55 mmol, 72 mL, 7.43 eq) and H₂SO₄ (36 mL), the mixture was stirred at 70° C. for 1 h. The reaction mixture was quenched by addition of saturated aqueous NaHCO₃ at 0° C., and then diluted with water (100 mL) and extracted with ethyl acetate (200 mL×2). The combined organic layers were washed with brine (200 mL), dried by anhydrous Na₂SO₄, filtered and concentrated in vacuum. The residue was purified by flash silica gel chromatography (SiO₂, petroleum ether/ethyl acetate=1/0 to 20/1). To afford 1-bromo-2,4-difluoro-3-nitrobenzene (2, 16 g, 67.23 mmol, 53% yield) as colorless oil.

¹H NMR (400 MHz, d₆-DMSO) δ 8.17 (ddd, J=5.6, 7.6, 9.2 Hz, 1H), 7.54 (dt, J=2.0, 9.6 Hz, 1H).

### Step 2: 2,6-bis(benzyloxy)-N-(4-bromo-3-fluoro-2-nitrophenyl)pyridin-3-amine (4)

To mixture of 2,6-bis(benzyloxy)pyridin-3-amine (3, 3 g, 9.79 mmol, 1 eq) and 1-bromo-2,4-difluoro-3-nitrobenzene (2, 2.80 g, 11.75 mmol, 7.70 mL, 1.2 eq) in DMF (30 mL) was added KF (682.69 mg, 11.75 mmol, 1.2 eq). The mixture was stirred at 130° C. for 16 h. The reaction mixture was filtered and concentrated in vacuum. The residue was purified by reversed phase flash (flow: 100 mL/min; gradient: from 5-100% MeCN in water (0.1% TFA) over 60 min; column: 330 g Flash Column Welch Ultimate XB_C18 20-40 μm, 120 Å) to afford 2,6-bis(benzyloxy)-N-(4-bromo-3-fluoro-2-nitrophenyl)pyridin-3-amine (4, 3.3 g, 6.23 mmol, 64% yield) as a yellow solid.

LCMS (ESI): m/z 526.3 [M+H]⁺

¹H NMR (400 MHz, CDCl3) δ 8.16 (s, 1H), 7.45 (d, J=8.4 Hz, 1H), 7.40-7.27 (m, 10H), 6.51 (dd, J=1.6, 9.2 Hz, 1H), 6.41 (d, J=8.0 Hz, 1H), 5.34 (d, J=15.2 Hz, 4H).

### Step 3: N¹-(2,6-bis(benzyloxy)pyridin-3-yl)-4-bromo-3-fluorobenzene-1,2-diamine (5)

To a solution of 2,6-bis(benzyloxy)-N-(4-bromo-3-fluoro-2-nitrophenyl)pyridin-3-amine (4, 3 g, 5.72 mmol, 1 eq) in EtOH (30 mL) and H₂O (30 mL) was added Fe (1.60 g, 28.61 mmol, 203.27 μL, 5 eq) and NH₄Cl (1.53 g, 28.61 mmol, 5 eq). The mixture was stirred at 70° C. for 16 h. The reaction mixture was filtered and the filtrate was concentrated in vacuum to remove ethanol. The mixture was poured into water (50 mL) and extracted with ethyl acetate (50 mL×2). Organic phases were combined and washed with brine (50 mL), dried by anhydrous Na₂SO₄, filtered and concentrated in vacuum. The residue was purified by flash silica gel chromatography (SiO₂, petroleum ether/ethyl acetate=1/0 to 10/1) to afford N¹-(2,6-bis(benzyloxy)pyridin-3-yl)-4-bromo-3-fluorobenzene-1,2-diamine (5, 1.8 g, 3.64 mmol, 64% yield) as a yellow solid.

LCMS (ESI): m/z 496.3 [M+H]⁺

### Step 4: 1-(2,6-bis(benzyloxy)pyridin-3-yl)-5-bromo-4-fluoro-1H-benzo[d]imidazol-2 (3H)-one (6)

To a solution of N¹-(2,6-bis(benzyloxy)pyridin-3-yl)-4-bromo-3-fluorobenzene-1,2-diamine (5, 1.8 g, 2.88 mmol, 1 eq) and Py (2.28 g, 28.76 mmol, 2.33 mL, 10 eq) in DCM (20 mL) was added dropwise a solution of triphosgene (1.71 g, 5.75 mmol, 2 eq) in DCM (20 mL) at 0˜10° C. The mixture was stirred at 20° C. for 16 h. The reaction mixture was poured into water (50 mL) and extracted with DCM (3×50 mL). Organic phases were combined and washed with brine (50 mL), dried by anhydrous Na₂SO₄, filtered and concentrated in vacuum. The residue was purified by flash silica gel chromatography (SiO₂, petroleum ether/ethyl acetate=1/0 to 3/1) to afford 1-(2,6-bis(benzyloxy)pyridin-3-yl)-5-bromo-4-fluoro-1H-benzo[d]imidazol-2 (3H)-one (6, 1.3 g, 2.50 mmol, 87% yield) a white solid.

LCMS (ESI): m/z 522.0 [M+H]⁺

¹H NMR (400 MHz, CDCl3) δ 7.55-7.45 (m, 1H), 7.40-7.19 (m, 8H), 7.18-6.97 (m, 3H), 6.50-6.21 (m, 2H), 5.47-5.16 (m, 4H)

### Step 5: 1-(2,6-bis(benzyloxy)pyridin-3-yl)-5-bromo-4-fluoro-3-methyl-1H-benzo[d]imidazol-2 (3H)-one (7)

To a solution of 1-(2,6-bis(benzyloxy)pyridin-3-yl)-5-bromo-4-fluoro-1H-benzo[d]imidazol-2 (3H)-one (6, 1.2 g, 2.31 mmol, 1 eq) in DMF (12 mL) were added Cs₂CO₃ (1.50 g, 4.61 mmol, 2 eq) and Mel (654.66 mg, 4.61 mmol, 287.13 μL, 2 ec). The mixture was stirred at 70° C. for 16 h. The reaction mixture was poured into water (20 mL) and extracted with ethyl acetate (20 mL×2). Organic phases were combined and washed with brine (30 mL), dried by anhydrous Na₂SO₄, filtered and concentrated in vacuum. The residue was purified by flash silica gel chromatography (SiO₂, petroleum ether/ethyl acetate=1/0 to 10/1) to afford 1-(2,6-bis(benzyloxy)pyridin-3-yl)-5-bromo-4-fluoro-3-methyl-1H-benzo[d]imidazol-2 (3H)-one (7, 1 g, 1.85 mmol, 80% yield) as a white solid.

LCMS (ESI): m/z 536.0 [M+H]⁺

¹H NMR (400 MHz, CDCl3) δ 7.59 (d, J=8.4 Hz, 1H), 7.50-7.32 (m, 5H), 7.30-7.27 (m, 2H), 7.27-7.25 (m, 1H), 7.25-7.19 (m, 2H), 7.25-7.19 (m, 1H), 6.53 (d, J=8.4 Hz, 1H), 6.36 (d, J=8.4 Hz, 1H), 5.51-5.42 (m, 1H), 5.37 (s, 2H), 5.31-5.22 (m, 1H), 3.67 (d, J=1.6 Hz, 3H).

### Step 6: tert-butyl 4-(1-(2,6-bis(benzyloxy)pyridin-3-yl)-4-fluoro-3-methyl-2-oxo-2,3-dihydro-1H-benzo[d]imidazol-5-yl)piperazine-1-carboxylate (9)

A mixture of 1-(2,6-bis(benzyloxy)pyridin-3-yl)-5-bromo-4-fluoro-3-methyl-1H-benzo[d]imidazol-2 (3H)-one (7, 330 mg, 617.54 μmol, 1 eq), tert-butylpiperazine-1-carboxylate (8, 230.04 mg, 1.24 mmol, 2 eq), Pd-PEPPSI-IHeptCl (30.01 mg, 30.88 μmol, 0.05 eq) and Cs₂CO₃ (603.62 mg, 1.85 mmol, 3 eq) in dioxane (3 mL) was degassed and purged with N₂ 3 times, and then the mixture was stirred at 90° C. for 16 hr under N₂ atmosphere. The mixture was filtered and concentrated in vacuum. The residue was purified by flash silica gel chromatography (SiO₂, petroleum ether/ethyl acetate=1/0 to 3/1) to afford tert-butyl 4-(1-(2,6-bis(benzyloxy)pyridin-3-yl)-4-fluoro-3-methyl-2-oxo-2,3-dihydro-1H-benzo[d]imidazol-5-yl)piperazine-1-carboxylate (9, 300 mg, 464.27 μmol, 75% yield) as a yellow solid.

LCMS (ESI): m/z 640.5 [M+H]⁺

¹H NMR (400 MHz, CDCl3) δ 7.59 (d, J=8.4 Hz, 1H), 7.45-7.34 (m, 5H), 7.27-7.21 (m, 5H), 6.59 (t, J=8.0 Hz, 1H), 6.52 (d, J=8.4 Hz, 1H), 6.38 (d, J=8.4 Hz, 1H), 5.49-5.43 (m, 1H), 5.35 (s, 2H), 5.32-5.27 (m, 1H), 3.67-3.61 (m, 7H), 3.00 (d, J=4.4 Hz, 4H), 1.50 (s, 9H).

### Step 7: 1-(2,6-bis(benzyloxy)pyridin-3-yl)-4-fluoro-3-methyl-5-(piperazin-1-yl)-1H-benzo[d]imidazol-2 (3H)-one (10)

To a solution of tert-butyl 4-(1-(2,6-bis(benzyloxy)pyridin-3-yl)-4-fluoro-3-methyl-2-oxo-2,3-dihydro-1H-benzo[d]imidazol-5-yl)piperazine-1-carboxylate (9, 250 mg, 390.80 μmol, 1 eq) in ethyl acetate (2.5 mL) was added HCl/ethyl acetate (4 M, 2.50 mL) at 0° C. The mixture was stirred at 20° C. for 1 h. The reaction mixture was filtered to afford 1-(2,6-bis(benzyloxy)pyridin-3-yl)-4-fluoro-3-methyl-5-(piperazin-1-yl)-1H-benzo[d]imidazol-2 (3H)-one (10, 200 mg, 336.77 μmol, 86% yield, HCl salt) as a white solid. The crude product was used in the next step without further purification.

LCMS (ESI): m/z 540.5 [M+H]⁺

### 3-[5-[4-(2-aminoethyl)-1-piperidyl]-6-fluoro-3-methyl-2-oxo-benzimidazol-1-yl]piperidine-2,6-dione (5)

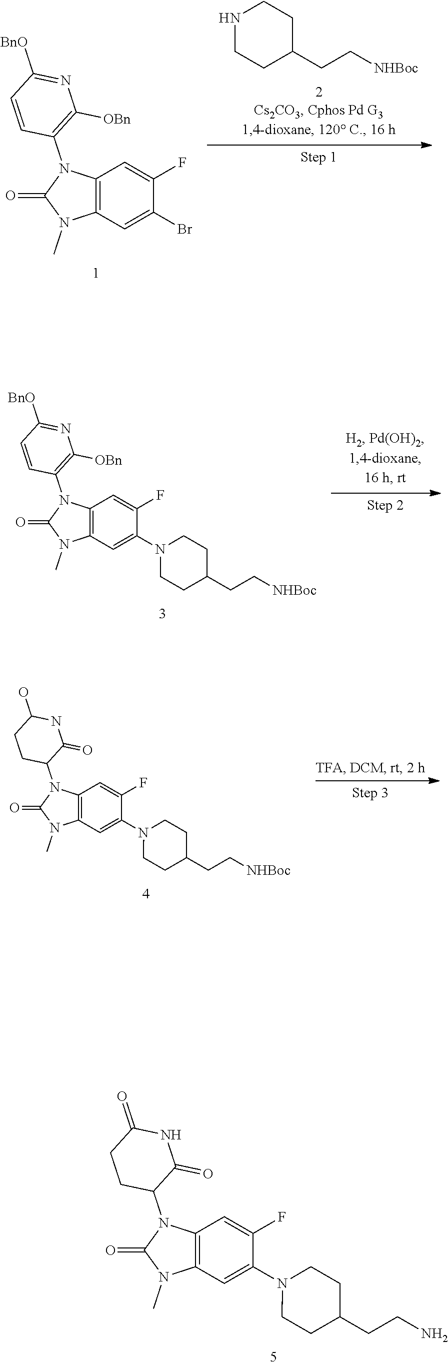 [SMILES: Cn1c(=O)n(-c2ccc(OCc3ccccc3)nc2OCc2ccccc2)c2cc(F)c(Br)cc21 ; CC(C)(C)OC(=O)NCCC1CCNCC1 ; Cn1c(=O)n(-c2ccc(OCc3ccccc3)nc2OCc2ccccc2)c2cc(F)c(N3CCC(CCNC(=O)OC(C)(C)C)CC3)cc21 ; Cn1c(=O)n(C2CCC(O)NC2=O)c2cc(F)c(N3CCC(CCNC(=O)OC(C)(C)C)CC3)cc21 ; Cn1c(=O)n(C2CCC(=O)NC2=O)c2cc(F)c(N3CCC(CCN)CC3)cc21]

### Step 1: tert-butyl N-[2-[1-[1-(2,6-dibenzyloxy-3-pyridyl)-6-fluoro-3-methyl-2-oxo-benzimidazol-5-yl]-4-piperidyl]ethyl]carbamate (3)

Into a 25 mL pressure tube containing a well-stirred solution of 5-bromo-1-(2,6-dibenzyloxy-3-pyridyl)-6-fluoro-3-methyl-benzimidazol-2-one (1, 500 mg, 865.22 μmol) and tert-butyl N-[2-(4-piperidyl)ethyl]carbamate (2, 395.11 mg, 1.73 mmol) in anhydrous 1,4-dioxane (8 mL) was added cesium carbonate (845.71 mg, 2.60 mmol) at room temperature. The mixture was degassed by bubbling nitrogen gas for 10 min. Then, CPhos Pd G3 (69.77 mg, 86.52 μmol) was added and stirred at 120° C. After 16 h, the reaction mixture was passed through Celite bed, filtrate was concentrated under reduced pressure to get the crude compound, which was purified by flash silica gel (230-400 mesh) column chromatography (70% EtOAc in pet ether) to afford tert-butyl N-[2-[1-[1-(2,6-dibenzyloxy-3-pyridyl)-6-fluoro-3-methyl-2-oxo-benzimidazol-5-yl]-4-piperidyl]ethyl]carbamate (3, 188 mg, 270.78 μmol, 31% yield) as a yellow solid.

LCMS (ES+): m/z 682.2 [M+H]⁺

### Step 2: tert-butyl N-[2-[1-[1-(2,6-dioxo-3-piperidyl)-6-fluoro-3-methyl-2-oxo-benzimidazol-5-yl]-4-piperidyl]ethyl]carbamate (4)

Into a 25 mL single-neck round-bottom flask containing a well-stirred solution of tert-butyl N-[2-[1-[1-(2,6-dibenzyloxy-3-pyridyl)-6-fluoro-3-methyl-2-oxo-benzimidazol-5-yl]-4-piperidyl]ethyl]carbamate (3, 100 mg, 144.91 μmol) in anhydrous 1,4-dioxane (2.5 mL) was added 20 wt. % palladium hydroxide on carbon (82.92 mg, 118.09 μmol, 20% purity) at room temperature. The suspension was stirred at room temperature under hydrogen bladder pressure for 16 h. The reaction mixture was passed through Celite, the filtrate was concentrated under reduced pressure to get crude compound which was purified by reverse phase column chromatography [Silicycle C18 column; Mobile phase A: 0.1% formic acid in water; Mobile phase B: MeCN] to get tert-butyl N-[2-[1-[1-(2,6-dioxo-3-piperidyl)-6-fluoro-3-methyl-2-oxo-benzimidazol-5-yl]-4-piperidyl]ethyl]carbamate (4, 50 mg, 88.51 μmol, 61% yield, Formic acid salt) as an off-white solid.

LCMS (ES+): m/z 504.2 [M+H]⁺

### Step 3: 3-[5-[4-(2-aminoethyl)-1-piperidyl]-6-fluoro-3-methyl-2-oxo-benzimidazol-1-yl]piperidine-2,6-dione (5)

Into a 25 mL single-neck round-bottom flask containing a well-stirred solution of tert-butyl N-[2-[1-[1-(2,6-dioxo-3-piperidyl)-6-fluoro-3-methyl-2-oxo-benzimidazol-5-yl]-4-piperidyl]ethyl]carbamate (4, 160 mg, 243.26 μmol) in anhydrous DCM (3 mL) was added TFA (1.48 g, 12.98 mmol, 1 mL) at room temperature. The resulting solution was stirred at room temperature for 2 h. The volatiles were removed from the reaction mixture to get the crude compound which was purified by reverse phase column chromatography [Silicycle C18 column; Mobile phase A: 0.1% TFA in water; Mobile phase B: MeCN] to get 3-[5-[4-(2-aminoethyl)-1-piperidyl]-6-fluoro-3-methyl-2-oxo-benzimidazol-1-yl]piperidine-2,6-dione (5, 105 mg, 202.50 μmol, 83% yield, TFA salt) as an off-white solid.

LCMS (ES+): m/z 404.1 [M+H]⁺

### 3-[5-[1-(2-aminoethyl)-3,3-difluoro-4-piperidyl]-3-methyl-2-oxo-benzimidazol-1-yl]piperidine-2,6-dione (4)

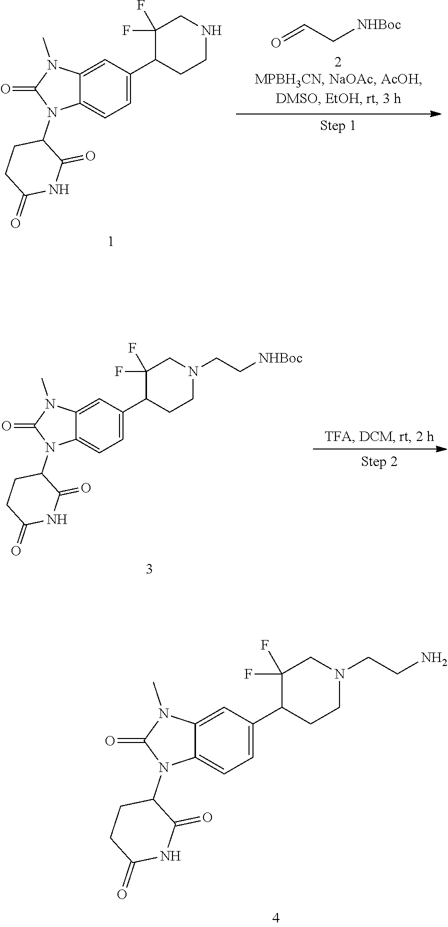 [SMILES: Cn1c(=O)n(C2CCC(=O)NC2=O)c2ccc(C3CCNCC3(F)F)cc21 ; CC(C)(C)OC(=O)NCC=O ; Cn1c(=O)n(C2CCC(=O)NC2=O)c2ccc(C3CCN(CCNC(=O)OC(C)(C)C)CC3(F)F)cc21 ; Cn1c(=O)n(C2CCC(=O)NC2=O)c2ccc(C3CCN(CCN)CC3(F)F)cc21]

### Step 1: tert-butyl N-[2-[4-[1-(2,6-dioxo-3-piperidyl)-3-methyl-2-oxo-benzimidazol-5-yl]-3,3-difluoro-1-piperidyl]ethyl]carbamate (3)

Into a 25 mL single-neck round-bottom flask containing a well-stirred solution of 3-[5-(3,3-difluoro-4-piperidyl)-3-methyl-2-oxo-benzimidazol-1-yl]piperidine-2,6-dione (1, 170 mg, 307.27 μmol, TFA salt), tert-butyl N-(2-oxoethyl)carbamate (2, 97.83 mg, 614.55 μmol) in anhydrous ethanol (1.5 mL) and DMSO (1.5 mL) were added acetic acid (223.13 mg, 3.72 mmol, 212.50 μL) and anhydrous sodium acetate (126.03 mg, 1.54 mmol) at room temperature. The mixture was stirred at room temperature for 1 h. Then, MP-Cyano borohydride (306 mg, 614.55 μmol, 2 mmol/g) was added and stirring continued for 3 h. The reaction mixture was passed through a sintered funnel, filtrate was concentrated under reduced pressure to get the crude compound, which was purified by reverse phase column chromatography [Silicycle C18 column; Mobile phase A: 0.1% formic acid in water; Mobile phase B: MeCN] to get tert-butyl N-[2-[4-[1-(2,6-dioxo-3-piperidyl)-3-methyl-2-oxo-benzimidazol-5-yl]-3,3-difluoro-1-piperidyl]ethyl]carbamate (3, 150 mg, 263.59 μmol, 86% yield, Formic acid salt) as a colorless solid.

LCMS (ES+): m/z 522.2 [M+H]⁺

### Step 2: 3-[5-[1-(2-aminoethyl)-3,3-difluoro-4-piperidyl]-3-methyl-2-oxo-benzimidazol-1-yl]piperidine-2,6-dione (4)

Into a 50 mL single neck round-bottom flask containing a well-stirred solution of tert-butyl N-[2-[4-[1-(2,6-dioxo-3-piperidyl)-3-methyl-2-oxo-benzimidazol-5-yl]-3,3-difluoro-1-piperidyl]ethyl]carbamate (3, 140 mg, 267.73 μmol) in anhydrous DCM (3 mL) was added TFA (740.00 mg, 6.49 mmol, 0.5 mL) at room temperature. The resulting solution was stirred at room temperature for 2 h. The volatiles were removed from the mixture to get 3-[5-[1-(2-aminoethyl)-3,3-difluoro-4-piperidyl]-3-methyl-2-oxo-benzimidazol-1-yl]piperidine-2,6-dione (4, 135 mg, 245.21 μmol, 92% yield, TFA salt) as white solid.

LCMS (ES+): m/z 422.2 [M+H]⁺

### 2-(4-(1-(2,6-dioxopiperidin-3-yl)-3-methyl-2-oxo-2,3-dihydro-1H-imidazo[4,5-b]pyridin-5-yl)phenyl)acetic acid (6)

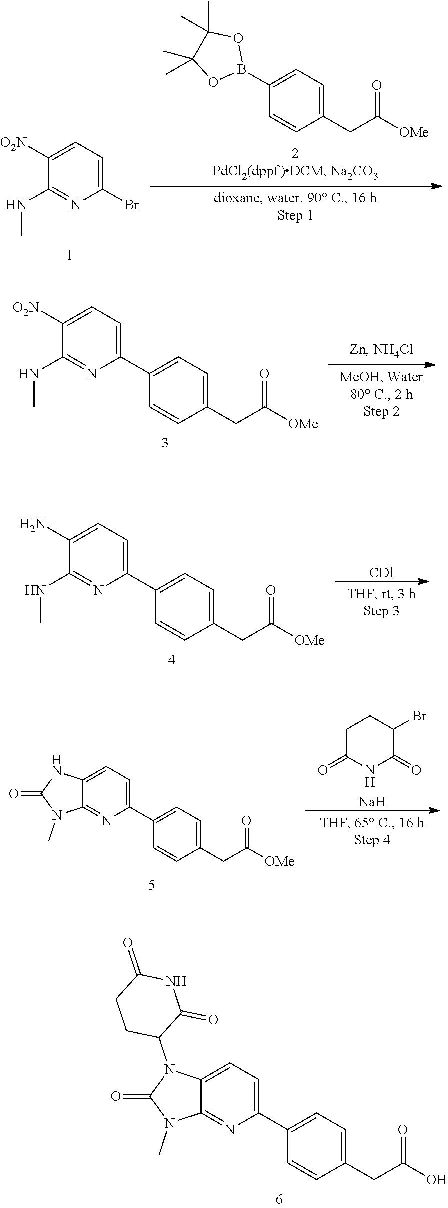

### Step 1: methyl 2-[4-[6-(methylamino)-5-nitro-2-pyridyl]phenyl]acetate (3)

Into a 100 mL sealed-tube containing a well-stirred solution of 6-bromo-N-methyl-3-nitro-pyridin-2-amine (1, 2 g, 8.27 mmol) and methyl 2-[4-(4,4,5,5-tetramethyl-1,3,2-dioxaborolan-2-yl)phenyl]acetate (2, 3.43 g, 12.41 mmol) in dioxane (40 mL) was added sodium carbonate (2.63 g, 24.82 mmol) in water (15 mL) at room temperature. The reaction mixture was purged with nitrogen gas for 5 min. Later [1,1′-Bis (diphenylphosphino)ferrocene]dichloropalladium (II), complex with dichloromethane (675.74 mg, 827.46 μmol) was added and reaction mixture was heated at 90° C. for 16 h. After completion of starting material, reaction mixture was filtered through Celite and washed thoroughly with EtOAc (25 mL) and concentrated under reduced pressure to get the crude material that was purified by silica gel (230-400 mesh) flash column chromatography (20% EtOAc in Pet ether) to obtain methyl 2-[4-[6-(methylamino)-5-nitro-2-pyridyl]phenyl]acetate (3, 2.5 g, 8.03 mmol, 97% yield) as a yellow solid.

LCMS (ES+): m/z 302.2 [M+H]⁺

### Step 2: methyl 2-[4-[5-amino-6-(methylamino)-2-pyridyl]phenyl]acetate (4)

Into a 500 mL single-neck round-bottom flask containing methyl 2-[4-[6-(methylamino)-5-nitro-2-pyridyl]phenyl]acetate (3, 4.2 g, 13.49 mmol) in methanol (50 mL) was added zinc dust (4.41 g, 67.45 mmol) and ammonium chloride (3.61 g, 67.45 mmol) in water (15 mL) and the resulting mixture was heated at 80° C. for 2 h. The reaction mixture was filtered through Celite, washed thoroughly with water (60 mL) and extracted with EtOAc (3×70 mL). The combined organic layers were dried over sodium sulfate and concentrated under reduced pressure to get the crude material that was purified by silica gel (230-400 mesh) column chromatography (60% EtOAc in Pet ether) to afford methyl 2-[4-[5-amino-6-(methylamino)-2-pyridyl]phenyl]acetate (4, 2.88 g, 10.19 mmol, 76% yield) as a light brown oil.

LCMS (ES+): m/z 272.2 [M+H]⁺

### Step 3: 2-[4-(3-methyl-2-oxo-1H-imidazo[4,5-b]pyridin-5-yl)phenyl]acetate (5)

Into a 100 mL round-bottom flask containing a well-stirred solution of methyl 2-[4-[5-amino-6-(methylamino)-2-pyridyl]phenyl]acetate (4, 3.00 g, 10.50 mmol) in THF (20 mL) was added carbonyldiimidazole (3.41 g, 21.01 mmol) at room temperature and the resulting mixture was stirred at room temperature for 3 h. The reaction mixture was filtered through Celite, washed thoroughly with water (60 mL) and extracted with EtOAc (3×70 mL). The combined organic layers were dried over sodium sulfate and concentrated under reduced pressure to get the crude material that was purified by silica gel (230-400 mesh) flash column chromatography (70% EtOAc in Pet ether) to afford methyl 2-[4-(3-methyl-2-oxo-1H-imidazo[4,5-b]pyridin-5-yl)phenyl]acetate (5, 1.16 g, 3.78 mmol, 36% yield) as an off-white solid.

LCMS (ES+): m/z 298.2 [M+H]⁺

### Step 4: 2-(4-(1-(2,6-dioxopiperidin-3-yl)-3-methyl-2-oxo-2,3-dihydro-1H-imidazo[4,5-b]pyridin-5-yl)phenyl)acetic acid (6)

To a suspension of sodium hydride (60% dispersion in mineral oil, 1.31 g, 34.26 mmol, 60% purity) in THF (6 mL) at 0° C. under inert atmosphere was added methyl 2-[4-(3-methyl-2-oxo-1H-imidazo[4,5-b]pyridin-5-yl)phenyl]acetate (5, 1.05 g, 3.43 mmol) in THF (6 mL). The reaction mixture was stirred at room temperature for 2 h. Subsequently, 3-bromopiperidine-2,6-dione (2.68 g, 13.70 mmol) was added at 0° C. and the resulting mixture was stirred at 65° C. for 16 h. The reaction mixture was quenched solution of 1.5 N HCl (15 mL) and extracted with EtOAc (3×50 mL). The combined organic layers were dried over sodium sulfate and concenrated under reduced pressure to get the crude material that was purified by reverse-phase preparative HPLC [Column: X-Select C18 (150×10) mm, 5 micron; Mobile phase A: 0.1% FA in water and Mobile phase B: MeCN] to afford the hydrolyzed product 2-(4-(1-(2,6-dioxopiperidin-3-yl)-3-methyl-2-oxo-2,3-dihydro-1H-imidazo[4,5-b]pyridin-5-yl)phenyl)acetic acid (6, 0.86 g, 77% yield, formic acid salt) as an off-white solid. [Note: The desired ester got hydrolyzed during the work-up to afford the acid]

LCMS (ES+): m/z 395.5 [M+H]⁺

### 3-[6-[1-(2-aminoethyl)-3,3-difluoro-4-piperidyl]-1-methyl-indazol-3-yl]piperidine-2,6-dione (8)

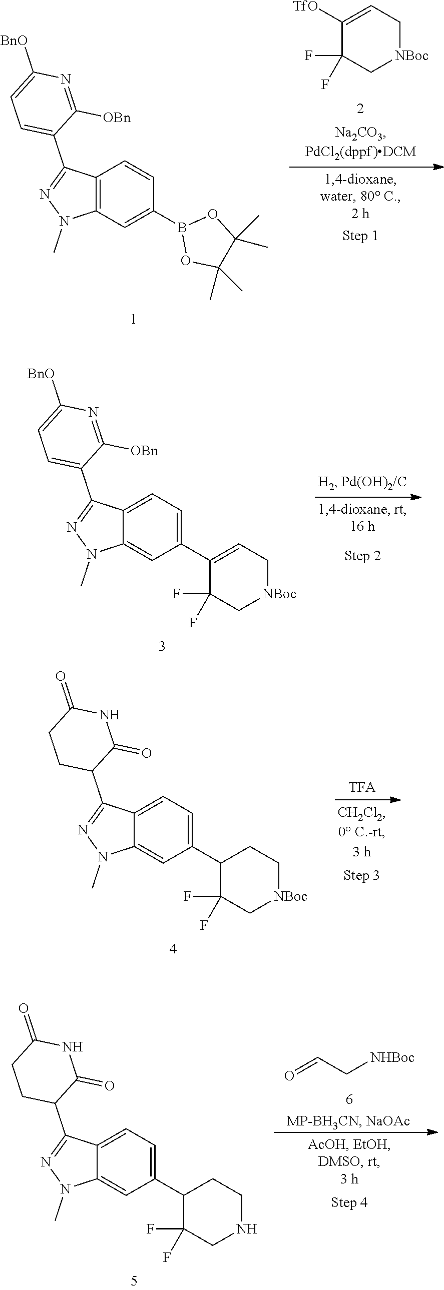

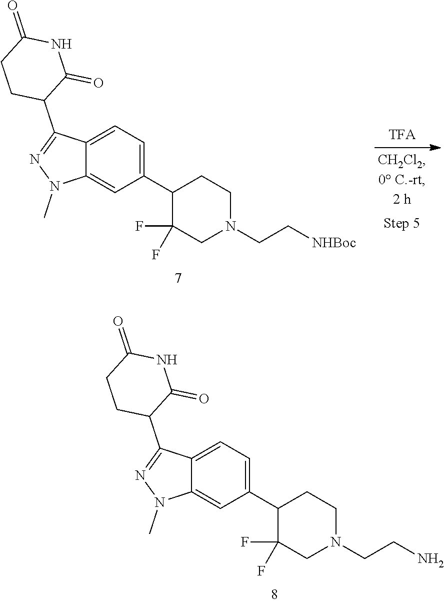 [SMILES: Cn1nc(C2CCC(=O)NC2=O)c2ccc(C3CCN(CCNC(=O)OC(C)(C)C)CC3(F)F)cc21 ; Cn1nc(C2CCC(=O)NC2=O)c2ccc(C3CCN(CCN)CC3(F)F)cc21]

### Step 1: tert-butyl 4-[3-(2,6-dibenzyloxy-3-pyridyl)-1-methyl-indazol-6-yl]-3,3-difluoro-2,6-dihydropyridine-1-carboxylate (3)

Into a 50 mL sealed-tube containing a well-stirred solution of 3-(2,6-dibenzyloxy-3-pyridyl)-1-methyl-6-(4,4,5,5-tetramethyl-1,3,2-dioxaborolan-2-yl) indazole (1, 500 mg, 913.32 μmol) and tert-butyl 3,3-difluoro-4-(trifluoromethylsulfonyloxy)-2,6-dihydropyridine-1-carboxylate (2, 436.09 mg, 1.19 mmol) in 1,4-dioxane (5 mL) and water (1 mL) was added sodium carbonate (290.41 mg, 2.74 mmol) at room temperature and then nitrogen gas was purged through the reaction mixture for 5 min. Then Pd(dppf)Cl₂·DCM (111.79 mg, 137.00 μmol) was added and the mixture was heated at 80° C. for 2 h. The reaction mixture was passed through Celite and washed with EtOAc (50 mL). The filtrate was concentrated under reduced pressure to get the crude compound that was purified by flash silica gel column chromatography (230-400 mesh silica gel; 25% EtOAc in Pet-ether) to afford tert-butyl 4-[3-(2,6-dibenzyloxy-3-pyridyl)-1-methyl-indazol-6-yl]-3,3-difluoro-2,6-dihydropyridine-1-carboxylate (3, 487 mg, 722.00 μmol, 79% yield) as a yellow sticky solid.

LCMS (ES+): m/z 639.2 [M+H]⁺

### Step 2: tert-butyl 4-[3-(2,6-dioxo-3-piperidyl)-1-methyl-indazol-6-yl]-3,3-difluoro-piperidine-1-carboxylate (4)

Into a 100 mL single-neck round-bottom flask containing a well-stirred solution of tert-butyl 4-[3-(2,6-dibenzyloxy-3-pyridyl)-1-methyl-indazol-6-yl]-3,3-difluoro-2,6-dihydropyridine-1-carboxylate (3, 480 mg, 706.43 μmol) in anhydrous 1,4-dioxane (5 mL) were added palladium hydroxide on carbon (20 wt. % 50% water, 545.66 mg, 777.08 μmol) at room temperature. The resultant reaction mixture was stirred under a hydrogen gas bladder for 16 h. The reaction mixture was filtered through Celite and washed successively with 1,4-dioxane (100 mL) and DMF (20 mL). The filtrate was evaporated under reduced pressure and the residue was triturated with diethyl ether (20 mL) to afford tert-butyl 4-[3-(2,6-dioxo-3-piperidyl)-1-methyl-indazol-6-yl]-3,3-difluoro-piperidine-1-carboxylate (4, 300 mg, 636.15 μmol, 90% yield) as an off-white solid.

LCMS (ES+): m/z 463.2 [M+H]⁺

### Step 3: 3-[6-(3,3-difluoro-4-piperidyl)-1-methyl-indazol-3-yl]piperidine-2,6-dione (5)

Into a 50 mL single neck round bottom flask containing a well-stirred solution of tert-butyl 4-[3-(2,6-dioxo-3-piperidyl)-1-methyl-indazol-6-yl]-3,3-difluoro-piperidine-1-carboxylate (4, 300 mg, 635.69 μmol) in dry DCM (4 mL) was added TFA (1.45 g, 12.71 mmol, 979.51 μL) at 0° C. The reaction mixture was stirred at room temperature for 3 h. The reaction mixture was concentrated under reduced pressure and triturated with diethyl ether (20 mL) to afford 3-[6-(3,3-difluoro-4-piperidyl)-1-methyl-indazol-3-yl]piperidine-2,6-dione (5, 295 mg, 606.97 μmol, 95% yield, TFA salt) as an off-white solid.

LCMS (ES+): m/z 363.2 [M+H]⁺

### Step 4: tert-butyl N-[2-[4-[3-(2,6-dioxo-3-piperidyl)-1-methyl-indazol-6-yl]-3,3-difluoro-1-piperidyl]ethyl]carbamate (7)

Into a 10 mL single-neck round-bottom flask containing well-stirred solution of 3-[6-(3,3-difluoro-4-piperidyl)-1-methyl-indazol-3-yl]piperidine-2,6-dione (5, 250 mg, 514.38 μmol, TFA salt) and tert-butyl N-(2-oxoethyl)carbamate (6, 163.76 mg, 1.03 mmol) in anhydrous ethanol (3 mL) and DMSO (3 mL) were added acetic acid (370.66 mg, 6.17 mmol, 353.01 μL) followed by anhydrous sodium acetate (210.97 mg, 2.57 mmol) at room temperature. The mixture was stirred at room temperature for 30 min. Subsequently, MP-Cyanoborohydride (1.0 g/2 mmol loading, 500 mg, 1.03 mmol) was added and stirring was continued for 3 h at room temperature. The reaction mixture was filtered through a sintered funnel, and concentrated under reduced pressure to get the crude compound that was purified by reverse-phase column chromatography [Silicycle C18 column, Mobile Phase A: 0.1% FA in water and Mobile Phase B: MeCN] to get the tert-butyl N-[2-[4-[3-(2,6-dioxo-3-piperidyl)-1-methyl-indazol-6-yl]-3,3-difluoro-1-piperidyl]ethyl]carbamate (7, 120 mg, 204.22 μmol, 40% yield, formic acid salt) as an off-white solid.

LCMS (ES+): m/z 506.2 [M+H]⁺

### Step 5: 3-[6-[1-(2-aminoethyl)-3,3-difluoro-4-piperidyl]-1-methyl-indazol-3-yl]piperidine-2,6-dione (8)

Into a 25 mL single-neck round-bottom flask containing well-stirred solution of tert-butyl N-[2-[4-[3-(2,6-dioxo-3-piperidyl)-1-methyl-indazol-6-yl]-3,3-difluoro-1-piperidyl]ethyl]carbamate (7, 120 mg, 237.36 μmol) in anhydrous DCM (2 mL) was added TFA (541.30 mg, 4.75 mmol, 365.74 μL) at 0° C. and the resulting mixture was stirred at room temperature for 2 h. The reaction mixture was concentrated under reduced pressure to get the crude compound that was purified by reverse phase [Column: Silicycle C18-120 g; Mobile Phase A: 0.1% HCOOH in water and Mobile Phase B: MeCN] to get the 3-[6-[1-(2-aminoethyl)-3,3-difluoro-4-piperidyl]-1-methyl-indazol-3-yl]piperidine-2,6-dione (8, 110 mg, 236.71 μmol, 99% yield, formic acid salt) as an off-white solid.

LCMS (ES+): m/z 406.2 [M+H]⁺

### 4-(4-(2-aminoethyl)piperidin-1-yl)-1-(2,6-bis(benzyloxy)pyridin-3-yl)-3-methyl-1H-benzo[d]imidazol-2 (3H)-one (9)

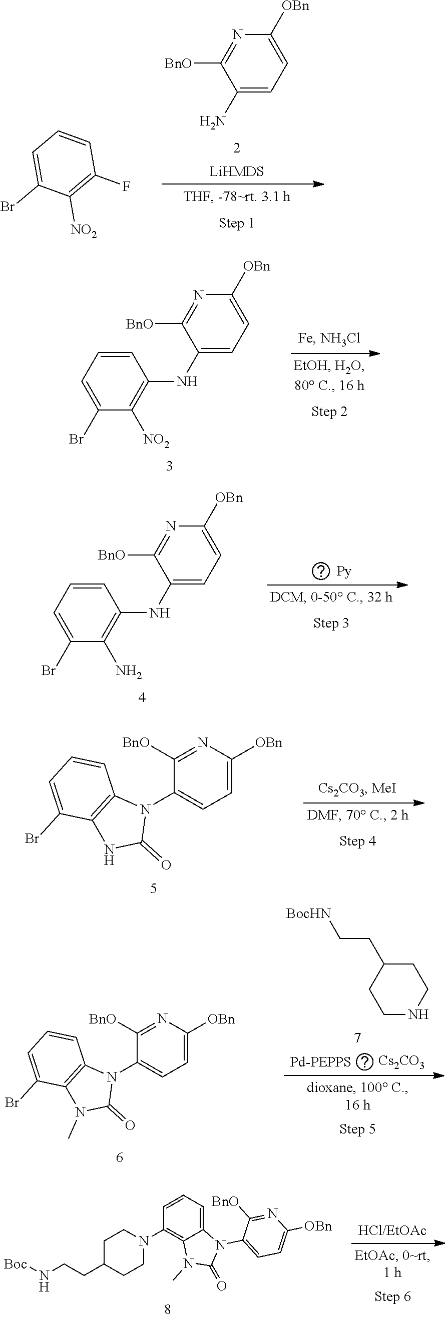

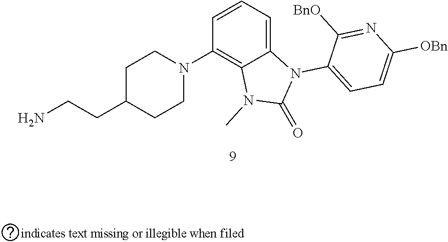 [SMILES: Cn1c(=O)n(-c2ccc(OCc3ccccc3)nc2OCc2ccccc2)c2cccc(N3CCC(CCN)CC3)c21]

### Step 1: 2,6-bis(benzyloxy)-N-(3-bromo-2-nitrophenyl)pyridin-3-amine (3)

To a solution of 2,6-bis(benzyloxy)pyridin-3-amine (2, 5 g, 16.32 mmol, 1 eq) in THF (50 mL) was added LiHMDS (1 M, 40.80 mL, 2.5 eq) at −78° C. The mixture was stirred at 0° C. for 0.1 h. Then solution of 1-bromo-3-fluoro-2-nitrobenzene (1, 3.77 g, 17.14 mmol, 7.70 mL, 1.05 eq) in THF (20 mL) was added. The mixture was stirred at 20° C. for 3 h. The reaction mixture was adjusted pH to 3 with 1N HCl aqueous. The reaction mixture was poured into water (100 mL) and extracted with ethyl acetate (100 mL×2). Organic phases were combined and washed with brine (100 mL), dried by anhydrous Na₂SO₄, filtered and concentrated in vacuum. The residue was purified by column chromatography (SiO₂, petroleum ether/ethyl acetate=1/0 to 5/1; petroleum ether/ethylacetate=5/1) to afford 2,6-bis(benzyloxy)-N-(3-bromo-2-nitrophenyl)pyridin-3-amine (3, 9.2 g, 17.99 mmol) as a yellow solid.

LCMS (ESI): m/z 508.2 [M+H]⁺

### Step 2: N¹-(2,6-bis(benzyloxy)pyridin-3-yl)-3-bromobenzene-1,2-diamine (4)

To a solution of 2,6-bis(benzyloxy)-N-(3-bromo-2-nitrophenyl)pyridin-3-amine (3, 3 g, 5.92 mmol, 1 eq) in EtOH (60 mL) and H₂O (10 mL) were added Fe (1.65 g, 29.62 mmol, 5 eq) and NH₄Cl (1.58 g, 29.62 mmol, 5 eq). The mixture was stirred at 80° C. for 16 h. The mixture was filtered and concentrated to removed ethanol, and extracted with ethyl acetate (60 mL×2). Organic phases were combined and washed with brine (60 mL), dried by anhydrous Na₂SO₄, filtered and concentrated in vacuum to afford N¹-(2,6-bis(benzyloxy)pyridin-3-yl)-3-bromobenzene-1,2-diamine (4, 2.7 g, 5.21 mmol, 88% yield) as red brown oil. The crude product was used into the next step without further purification.

LCMS (ESI): m/z 478.0 [M+H]⁺

¹H NMR (400 MHz, d₆-DMSO) δ 7.43-7.29 (m, 10H), 7.16 (d, J=8.4 Hz, 1H), 7.00 (dd, J=1.2, 8.0 Hz, 1H), 6.58 (dd, J=1.2, 8.0 Hz, 1H), 6.48-6.37 (m, 3H), 5.40 (s, 2H), 5.27 (s, 2H), 4.89 (s, 2H)

### Step 3: 1-(2,6-bis(benzyloxy)pyridin-3-yl)-4-bromo-1H-benzo[d]imidazol-2 (3H)-one (5)

To a solution of N¹-(2,6-bis(benzyloxy)pyridin-3-yl)-3-bromobenzene-1,2-diamine (4, 2.7 g, 4.48 mmol, 1 eq) and Py (3.54 g, 44.78 mmol, 3.62 mL, 10 eq) in DCM (30 mL) was added triphosgene (2.66 g, 8.96 mmol, 2 eq) at 0˜10° C. The mixture was stirred at 20° C. for 16 h. The mixture was stirred at 50° C. for 16 h. The reaction mixture was poured into water (100 mL) and extracted with DCM (50 mL×3). Organic phases were combined and washed with brine (50 mL), dried by anhydrous Na₂SO₄, filtered and concentrated in vacuum to afford 1-(2,6-bis(benzyloxy)pyridin-3-yl)-4-bromo-1H-benzo[d]imidazol-2 (3H)-one (5, 3.2 g, 4.33 mmol, 96% yield) as a yellow solid. The crude product was used into the next step without further purification.

LCMS (ESI): m/z 502.0 [M+H]⁺

### Step 4: 1-(2,6-bis(benzyloxy)pyridin-3-yl)-4-bromo-3-methyl-1H-benzo[d]imidazol-2 (3H)-one (6)

To a solution of 1-(2,6-bis(benzyloxy)pyridin-3-yl)-4-bromo-1H-benzo[d]imidazol-2 (3H)-one (5, 3.2 g, 4.33 mmol, 1 eq) in DMF (30 mL) were add Cs₂CO₃ (2.82 g, 8.66 mmol, 2 eq) and Mel (1.23 g, 8.66 mmol, 539.32 μL, 2 eq). The mixture was stirred at 70° C. for 2 h. The reaction mixture was poured into water (10 mL) and extracted with ethyl acetate (50 mL×2). Organic phases were combined and washed with brine (50 mL), dried by anhydrous Na₂SO₄, filtered and concentrated in vacuum. The residue was purified by flash silica gel chromatography (SiO₂, petroleum ether/ethyl acetate=1/0 to 2/1; petroleum ether/ethylacetate=3/1) to afford 1-(2,6-bis(benzyloxy)pyridin-3-yl)-4-bromo-3-methyl-1H-benzo[d]imidazol-2 (3H)-one (6, 1.9 g, 3.42 mmol, 79% yield) as a brown solid.

LCMS (ESI): m/z 518.1 [M+H]⁺

¹H NMR (400 MHz, d₆-DMSO) δ 7.83 (d, J=8.4 Hz, 1H), 7.46-7.34 (m, 5H), 7.30-7.24 (m, 6H), 6.93 (t, J=8.0 Hz, 1H), 6.69 (dd, J=0.8, 7.6 Hz, 1H), 6.63 (d, J=8.4 Hz, 1H), 5.43-5.34 (m, 4H), 3.68 (s, 3H).

### Step 5: tert-butyl (2-(1-(1-(2,6-bis(benzyloxy)pyridin-3-yl)-3-methyl-2-oxo-2,3-dihydro-1H-benzo[d]imidazol-4-yl)piperidin-4-yl)ethyl)carbamate (8)

A mixture of 1-(2,6-bis(benzyloxy)pyridin-3-yl)-4-bromo-3-methyl-1H-benzo[d]imidazol-2 (3H)-one (6, 500 mg, 968.27 μmol, 1 eq), tert-butyl (2-(piperidin-4-yl)ethyl)carbamate (7, 287.41 mg, 1.26 mmol, 1.3 eq), Cs₂CO₃ (946.44 mg, 2.90 mmol, 3 eq) and Pd-PEPPSI-IHeptCl (47.06 mg, 48.41 μmol, 0.05 eq) in dioxane (5 mL) was degassed and purged with N₂ 3 times, and then the mixture was stirred at 100° C. for 16 hr under N₂ atmosphere. The mixture was concentrated in vacuum. The residue was purified by flash silica gel chromatography (SiO₂, petroleum ether/ethyl acetate=1/0 to 4/1; petroleum ether/ethylacetate=1/1) to afford tert-butyl (2-(1-(1-(2,6-bis(benzyloxy)pyridin-3-yl)-3-methyl-2-oxo-2,3-dihydro-1H-benzo[d]imidazol-4-yl)piperidin-4-yl)ethyl)carbamate (8, 380 mg, 543.84 μmol, 56% yield) as yellow oil.

LCMS (ESI): m/z 664.4 [M+H]⁺

### Step 6: 4-(4-(2-aminoethyl)piperidin-1-yl)-1-(2,6-bis(benzyloxy)pyridin-3-yl)-3-methyl-1H-benzo[d]imidazol-2 (3H)-one (9)

To a solution of tert-butyl (2-(1-(1-(2,6-bis(benzyloxy)pyridin-3-yl)-3-methyl-2-oxo-2,3-dihydro-1H-benzo[d]imidazol-4-yl)piperidin-4-yl)ethyl)carbamate (8, 380 mg, 572.46 μmol, 1 eq) in EtOAc (4 mL) was added HCl/EtOAc (4 M, 4 mL) at 0° C. The mixture was stirred at 20° C. for 1 h. The reaction mixture was filtered to afford filter cake. The residue was purified by prep-HPLC (flow: 50 mL/min; gradient: from 0-45% MeCN in water (0.1% TFA) over 30 min; column: 40 g Flash Column Welch Ultimate XB_C18 20-40 μm; 120 Å) to afford 4-(4-(2-aminoethyl)piperidin-1-yl)-1-(2,6-bis(benzyloxy)pyridin-3-yl)-3-methyl-1H-benzo[d]imidazol-2 (3H)-one (9, 130 mg, 226.01 μmol, 39% yield) as a white solid.

LCMS (ESI): m/z 564.5 [M+H]⁺

### (2R)-3-(benzyloxy)-2-(4-(1-(2,6-dioxopiperidin-3-yl)-3-ethyl-2-oxo-2,3-dihydro-1H-benzo[d]imidazol-5-yl)piperidin-1-yl)propanoic acid (8)

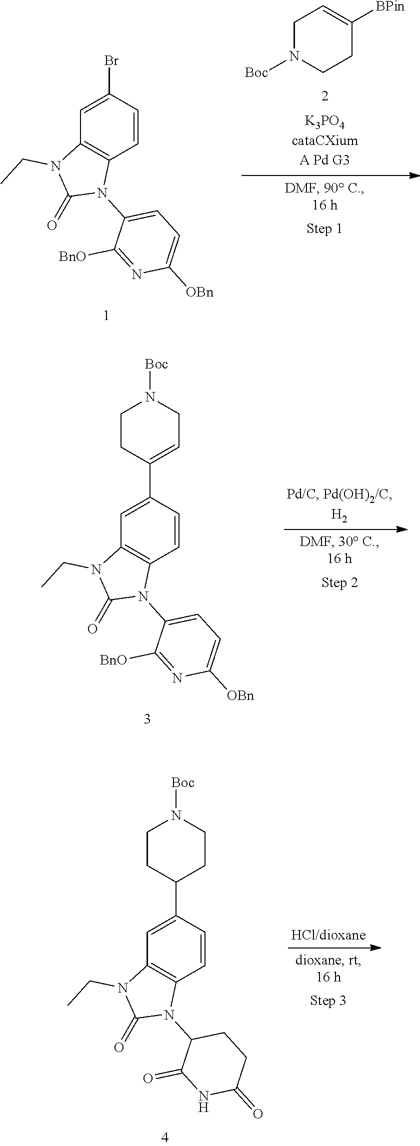

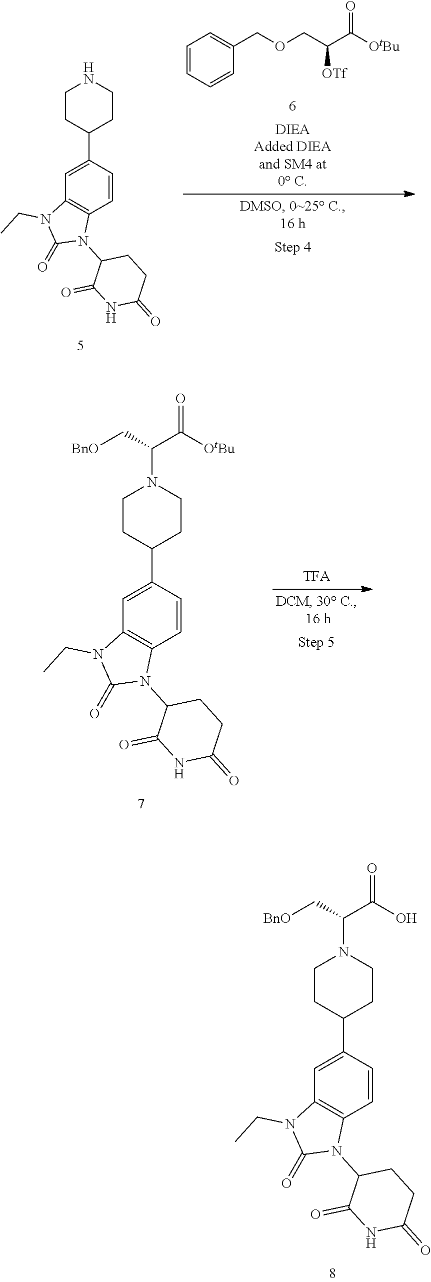 [SMILES: CCn1c(=O)n(C2CCC(=O)NC2=O)c2ccc(C3CCNCC3)cc21 ; CC(C)(C)OC(=O)[C@H](COCc1ccccc1)OS(=O)(=O)C(F)(F)F ; CCn1c(=O)n(C2CCC(=O)NC2=O)c2ccc(C3CCN([C@H](COCc4ccccc4)C(=O)OC(C)(C)C)CC3)cc21 ; CCn1c(=O)n(C2CCC(=O)NC2=O)c2ccc(C3CCN([C@H](COCc4ccccc4)C(=O)O)CC3)cc21]

### Step 1: tert-butyl 4-(1-(2,6-bis(benzyloxy)pyridin-3-yl)-3-ethyl-2-oxo-2,3-dihydro-1H-benzo[d]imidazol-5-yl)-5,6-dihydropyridine-1 (2H)-carboxylate (3)

To a mixture of 1-(2,6-bis(benzyloxy)pyridin-3-yl)-5-bromo-3-ethyl-1H-benzo[d]imidazol-2 (3H)-one (1, 3 g, 5.66 mmol, 1 eq), tert-butyl 4-(4,4,5,5-tetramethyl-1,3,2-dioxaborolan-2-yl)-5,6-dihydropyridine-1 (2H)-carboxylate (2, 1.75 g, 5.66 mmol, 1 eq) and K₃PO₄ (3.60 g, 16.97 mmol, 3 eq) in DMF (30 mL) was added cataCXium A Pd G3 (412.48 mg, 565.60 μmol, 0.1 eq) under N₂, the mixture was stirred at 90° C. for 16 h under N₂. The mixture was filtered and concentrated in vacuum. The residue was purified by column chromatography (SiO₂, Petroleum ether/Ethyl acetate=100/1 to 1/1) to afford tert-butyl 4-(1-(2,6-bis(benzyloxy)pyridin-3-yl)-3-ethyl-2-oxo-2,3-dihydro-1H-benzo[d]imidazol-5-yl)-5,6-dihydropyridine-1 (2H)-carboxylate (3, 3.1 g, 4.85 mmol, 86% yield) as yellow solid.

LCMS (ESI): m/z 633.2 [M+H]⁺

### Step 2: tert-butyl 4-(1-(2,6-dioxopiperidin-3-yl)-3-ethyl-2-oxo-2,3-dihydro-1H-benzo[d]imidazol-5-yl)piperidine-1-carboxylate (4)

To a solution of tert-butyl 4-(1-(2,6-bis(benzyloxy)pyridin-3-yl)-3-ethyl-2-oxo-2,3-dihydro-1H-benzo[d]imidazol-5-yl)-5,6-dihydropyridine-1 (2H)-carboxylate (3, 3.1 g, 4.90 mmol, 1 eq) in DMF (40 mL) was added Pd/C (500 mg, 10% purity) and Pd(OH)₂/C (500 mg, 10% purity) under H₂ (15 Psi), the mixture was stirred at 30° C. for 16 h under H₂ (15 Psi) atmosphere. The mixture was filtered and concentrated in vacuum. The residue was purified by column chromatography (SiO₂, Petroleum ether/Ethyl acetate=100/1 to 0/1) to afford tert-butyl 4-(1-(2,6-dioxopiperidin-3-yl)-3-ethyl-2-oxo-2,3-dihydro-1H-benzo[d]imidazol-5-yl)piperidine-1-carboxylate (4, 2 g, 4.38 mmol, 89% yield) as yellow solid.

LCMS (ESI): m/z 457.0 [M+H]⁺

### Step 3: 3-(3-ethyl-2-oxo-5-(piperidin-4-yl)-2,3-dihydro-1H-benzo[d]imidazol-1-yl)piperidine-2,6-dione (5)

A mixture of tert-butyl 4-(1-(2,6-dioxopiperidin-3-yl)-3-ethyl-2-oxo-2,3-dihydro-1H-benzo[d]imidazol-5-yl)piperidine-1-carboxylate (4, 1 g, 2.19 mmol, 1 eq) and HCl/Dioxane (4 M, 10 mL) in dioxane (5 mL) was stirred at 20° C. for 2 h. The mixture was concentrated in vacuum to afford 3-(3-ethyl-2-oxo-5-(piperidin-4-yl)-2,3-dihydro-1H-benzo[d]imidazol-1-yl)piperidine-2,6-dione (5, 860 mg, 2.19 mmol, 99% yield, HCl salt) as yellow solid, which was used directly for next step.

LCMS (ESI): m/z 356.9 [M+H]⁺

### Step 4: (2R)-tert-butyl 3-(benzyloxy)-2-(4-(1-(2,6-dioxopiperidin-3-yl)-3-ethyl-2-oxo-2,3-dihydro-1H-benzo[d]imidazol-5-yl)piperidin-1-yl)propanoate (7)

To a solution of 3-(3-ethyl-2-oxo-5-(piperidin-4-yl)-2,3-dihydro-1H-benzo[d]imidazol-1-yl)piperidine-2,6-dione (5, 400 mg, 1.02 mmol, HCl salt, 1 eq) in DMSO (10 mL) was added DIPEA (394.75 mg, 3.05 mmol, 532.00 μL, 3 eq) and (S)-tert-butyl 3-(benzyloxy)-2-(((trifluoromethyl)sulfonyl)oxy)propanoate (6, 508.73 mg, 1.32 mmol, 1.3 eq) at 0° C. under N₂, then the mixture was stirred at 20° C. for 16 h. The mixture was poured into H₂O (50 mL). The mixture was extracted with EtOAc (30 mL). The organic phase was washed with brine (50 mL), dried with anhydrous Na₂SO₄, filtered and concentrated in vacuum. The residue was purified by column chromatography (SiO₂, Petroleum ether/Ethyl acetate=100/1 to 0/1) to afford (2R)-tert-butyl 3-(benzyloxy)-2-(4-(1-(2,6-dioxopiperidin-3-yl)-3-ethyl-2-oxo-2,3-dihydro-1H-benzo[d]imidazol-5-yl)piperidin-1-yl)propanoate (7,200 mg, 321.65 μmol, 32% yield) as yellow solid.

LCMS (ESI): m/z 591.3 [M+H]⁺

### Step 5: (2R)-3-(benzyloxy)-2-(4-(1-(2,6-dioxopiperidin-3-yl)-3-ethyl-2-oxo-2,3-dihydro-1H-benzo[d]imidazol-5-yl)piperidin-1-yl)propanoic acid (8)

To a solution of (2R)-tert-butyl 3-(benzyloxy)-2-(4-(1-(2,6-dioxopiperidin-3-yl)-3-ethyl-2-oxo-2,3-dihydro-1H-benzo[d]imidazol-5-yl)piperidin-1-yl)propanoate (7, 200 mg, 338.58 μmol, 1 eq) in DCM (2 mL) was added TFA (2.96 g, 25.96 mmol, 2 mL) at 0° C., the mixture was stirred at 20° C. for 16 h. The mixture was concentrated in vacuum to afford (2R)-3-(benzyloxy)-2-(4-(1-(2,6-dioxopiperidin-3-yl)-3-ethyl-2-oxo-2,3-dihydro-1H-benzo[d]imidazol-5-yl)piperidin-1-yl)propanoic acid (8, 219 mg, 337.64 μmol, 99% yield, TFA salt) as yellow oil, which was used directly for next step.

LCMS (ESI): m/z 535.1 [M+H]⁺

### (2S)-3-(benzyloxy)-2-(4-(1-(2,6-dioxopiperidin-3-yl)-3-ethyl-2-oxo-2,3-dihydro-1H-benzo[d]imidazol-5-yl)piperidin-1-yl)propanoic acid (4)

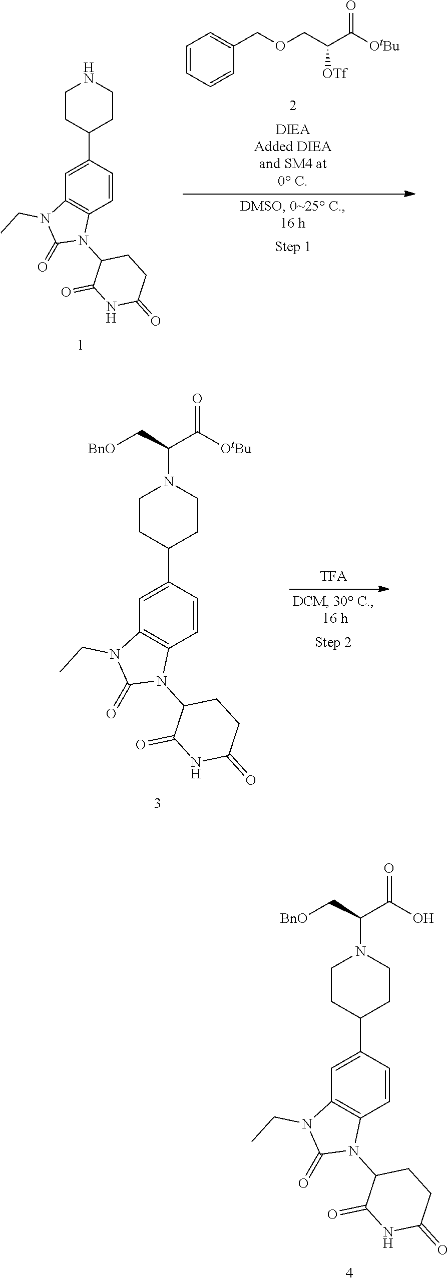 [SMILES: CCn1c(=O)n(C2CCC(=O)NC2=O)c2ccc(C3CCNCC3)cc21 ; CC(C)(C)OC(=O)[C@@H](COCc1ccccc1)OS(=O)(=O)C(F)(F)F ; CCn1c(=O)n(C2CCC(=O)NC2=O)c2ccc(C3CCN([C@@H](COCc4ccccc4)C(=O)OC(C)(C)C)CC3)cc21 ; CCn1c(=O)n(C2CCC(=O)NC2=O)c2ccc(C3CCN([C@@H](COCc4ccccc4)C(=O)O)CC3)cc21]

### Step 1: (2S)-tert-butyl 3-(benzyloxy)-2-(4-(1-(2,6-dioxopiperidin-3-yl)-3-ethyl-2-oxo-2,3-dihydro-1H-benzo[d]imidazol-5-yl)piperidin-1-yl)propanoate (3)

To a solution of 3-(3-ethyl-2-oxo-5-(piperidin-4-yl)-2,3-dihydro-1H-benzo[d]imidazol-1-yl)piperidine-2,6-dione (1, 400 mg, 1.02 mmol, HCl salt, 1 eq) in DMSO (10 mL) was added DIPEA (394.75 mg, 3.05 mmol, 532.00 μL, 3 eq) and (R)-tert-butyl 3-(benzyloxy)-2-(((trifluoromethyl)sulfonyl)oxy)propanoate (2, 508.73 mg, 1.32 mmol, 1.3 eq) at 0° C. under N₂, then the mixture was stirred at 20° C. for 16 h. The mixture was poured into H₂O (50 mL). The mixture was extracted with EtOAc (30 mL). The organic phase was washed with brine (50 mL), dried with anhydrous Na₂SO₄, filtered and concentrated in vacuum. The residue was purified by column chromatography (SiO₂, Petroleum ether/Ethyl acetate=100/1 to 0/1) to afford (2S)-tert-butyl 3-(benzyloxy)-2-(4-(1-(2,6-dioxopiperidin-3-yl)-3-ethyl-2-oxo-2,3-dihydro-1H-benzo[d]imidazol-5-yl)piperidin-1-yl)propanoate (3, 190 mg, 321.65 μmol, 32% yield) as yellow solid.

LCMS (ESI): m/z 591.1 [M+H]⁺

### Step 2: (2S)-3-(benzyloxy)-2-(4-(1-(2,6-dioxopiperidin-3-yl)-3-ethyl-2-oxo-2,3-dihydro-1H-benzo[d]imidazol-5-yl)piperidin-1-yl)propanoic acid (4)

To a solution of (2S)-tert-butyl 3-(benzyloxy)-2-(4-(1-(2,6-dioxopiperidin-3-yl)-3-ethyl-2-oxo-2,3-dihydro-1H-benzo[d]imidazol-5-yl)piperidin-1-yl)propanoate (3, 160 mg, 277.45 μmol, 1 eq) in DCM (4 mL) was added TFA (2.96 g, 25.96 mmol, 2 mL) at 0° C., the mixture was stirred at 20° C. for 16 h. The mixture was concentrated in vacuum to afford (2S)-3-(benzyloxy)-2-(4-(1-(2,6-dioxopiperidin-3-yl)-3-ethyl-2-oxo-2,3-dihydro-1H-benzo[d]imidazol-5-yl)piperidin-1-yl)propanoic acid (4, 176 mg, 277.34 μmol, 99% yield, TFA salt) as yellow oil, which was used directly for next step.

LCMS (ESI): m/z 535.3 [M+H]⁺

### 2-[(3S)-4-[1-(2,6-dioxo-3-piperidyl)-6-fluoro-3-methyl-2-oxo-benzimidazol-5-yl]-3-methyl-piperazin-1-yl]acetic acid (12)

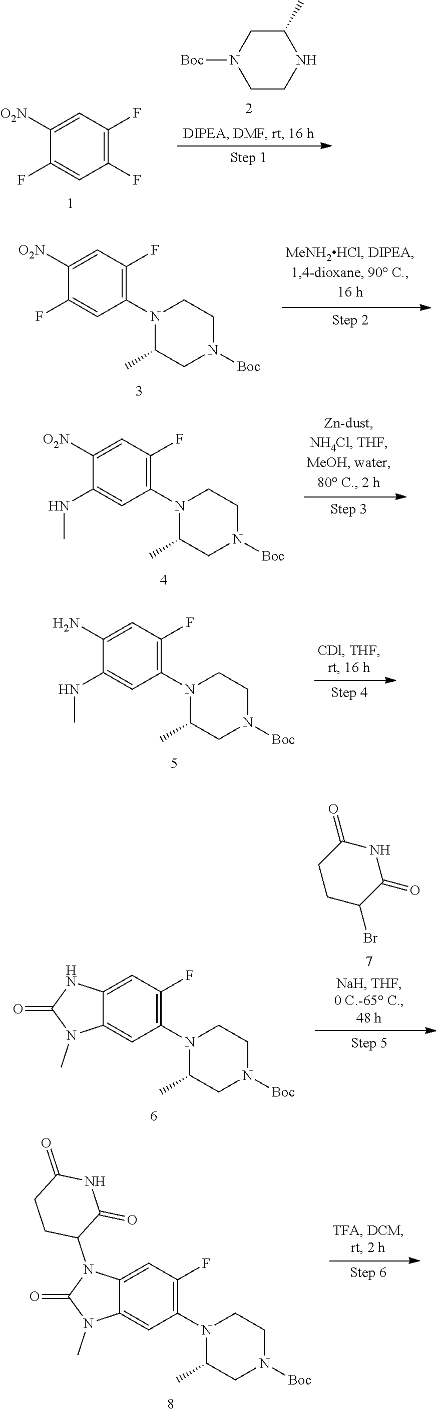

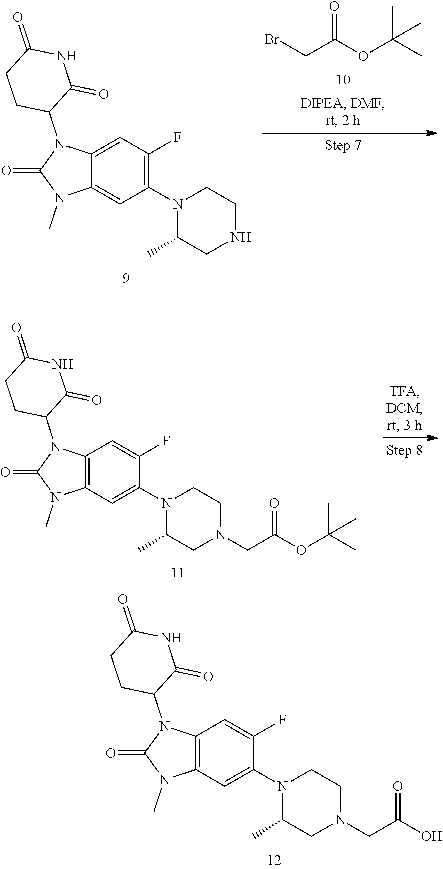

### Step 1: tert-butyl (3S)-4-(2,5-difluoro-4-nitro-phenyl)-3-methyl-piperazine-1-carboxylate (3)

Into a 50 mL single neck round-bottom flask containing a well-stirred solution of tert-butyl (3S)-3-methylpiperazine-1-carboxylate (2, 2.26 g, 11.29 mmol) in anhydrous DMF (8 mL) were added DIPEA (2.19 g, 16.94 mmol, 2.95 mL) and 1,2,4-trifluoro-5-nitro-benzene (1, 2 g, 11.29 mmol, 1.30 mL) and the reaction mixture was stirred at room temperature. After 16 h, the reaction mixture was diluted with ethyl acetate (50 mL) and washed with ice cold water (3×50 mL). The organic layer was dried over anhydrous Na₂SO₄, filtered and concentrated under reduced pressure to get tert-butyl (3S)-4-(2,5-difluoro-4-nitro-phenyl)-3-methyl-piperazine-1-carboxylate (3, 3.7 g, 9.32 mmol, 83% yield) as yellow gummy liquid.

LCMS (ES+): m/z 358.5 [M+H]⁺

### Step 2: tert-butyl (3S)-4-[2-fluoro-5-(methylamino)-4-nitro-phenyl]-3-methyl-piperazine-1-carboxylate (4)

Into a 250 mL pressure tube containing a well-stirred solution of tert-butyl (3S)-4-(2,5-difluoro-4-nitro-phenyl)-3-methyl-piperazine-1-carboxylate (3, 3.62 g, 9.12 mmol) in anhydrous 1,4-dioxane (40 mL) were added methylamine hydrochloride (923.35 mg, 13.68 mmol) and DIPEA (3.53 g, 27.35 mmol, 4.76 mL) at room temperature stirred at 90° C. After 16 h, the solvent was removed under reduced pressure and the residue was taken in ethyl acetate (100 mL). The organic layer was washed with water (2×100 mL), dried over anhydrous Na₂SO₄, filtered and concentrated under reduced pressure to get crude tert-butyl (3S)-4-[2-fluoro-5-(methylamino)-4-nitro-phenyl]-3-methyl-piperazine-1-carboxylate (4, 3.6 g, 7.76 mmol, 85% yield) as reddish gummy liquid.

LCMS (ES+): m/z 369.2 [M+H]⁺

### Step 3: tert-butyl (3S)-4-[4-amino-2-fluoro-5-(methylamino)phenyl]-3-methyl-piperazine-1-carboxylate (5)

Into a 250 mL single neck round bottom flask containing a well-stirred solution of tert-butyl (3S)-4-[2-fluoro-5-(methylamino)-4-nitro-phenyl]-3-methyl-piperazine-1-carboxylate (4, 3.6 g, 7.72 mmol) in THF (40 mL), methanol (30 mL) and water (10 mL) were added Zinc powder (2.52 g, 38.60 mmol) and ammonium chloride (2.06 g, 38.60 mmol) at room temperature. The suspension was stirred at 80° C. for 2 h. The reaction mixture was filtered through Celite, washed with ethyl acetate (150 mL). The organic layer was washed with water (3×150 mL), dried over anhydrous Na₂SO₄, filtered and concentrated under reduced pressure to get crude compound which was purified by flash silica gel (230-400 mesh) column chromatography (50-60% EtOAc in pet ether) to afford tert-butyl (3S)-4-[4-amino-2-fluoro-5-(methylamino)phenyl]-3-methyl-piperazine-1-carboxylate (5, 865 mg, 1.34 mmol, 17% yield) as a brownish solid. LCMS (ES+): m/z 338.2 [M+H]⁺

### Step 4: tert-butyl (3S)-4-(6-fluoro-3-methyl-2-oxo-1H-benzimidazol-5-yl)-3-methyl-piperazine-1-carboxylate (6)

Into a 50 mL single neck round-bottom flask containing a well-stirred solution of tert-butyl (3S)-4-[4-amino-2-fluoro-5-(methylamino)phenyl]-3-methyl-piperazine-1-carboxylate (5, 865 mg, 1.33 mmol) in anhydrous THF (8 mL) was added CDI (646.55 mg, 3.99 mmol) at room temperature. The resulting solution was stirred at room temperature. After 16 h, the reaction mixture was diluted with DCM (100 mL) and organic layer was washed with water (2×100 mL), dried over anhydrous Na₂SO₄, filtered and concentrated under reduced pressure. The crude compound was purified by reverse phase column chromatography [Silicycle C18 column; Mobile phase A: 0.1% Formic acid in water; Mobile phase B: MeCN] to get tert-butyl (3S)-4-(6-fluoro-3-methyl-2-oxo-1H-benzimidazol-5-yl)-3-methyl-piperazine-1-carboxylate (6, 348 mg, 843.55 μmol, 63% yield, Formic acid salt) as pink solid.

LCMS (ES+): m/z 365.2 [M+H]⁺

### Step 5: tert-butyl (3S)-4-[1-(2,6-dioxo-3-piperidyl)-6-fluoro-3-methyl-2-oxo-benzimidazol-5-yl]-3-methyl-piperazine-1-carboxylate (8)

Into a 100 mL single neck round-bottom flask containing a well-stirred solution of tert-butyl (3S)-4-(6-fluoro-3-methyl-2-oxo-1H-benzimidazol-5-yl)-3-methyl-piperazine-1-carboxylate (6, 348.00 mg, 843.55 μmol, Formic acid salt) in anhydrous THF (10 mL) was added 60% sodium hydride (in oil dispersion) (724.50 mg, 18.91 mmol, 60% purity) at 0° C. in portions with 5 min interval and the mixture was stirred at room temperature for 1 h. Then a solution of 3-bromopiperidine-2,6-dione (7, 1.50 g, 7.56 mmol) in anhydrous THF (5 mL) was added dropwise at 0° C. and stirred the reaction mixture at 65° C. for 48 h. The reaction mixture was cooled to 0° C. and quenched with saturated ammonium chloride solution. The aqueous layer was extracted with EtOAc (2×50 mL). The combined organic layer was dried over anhydrous Na₂SO₄, filtered and concentrated. The crude compound was purified by reverse phase column chromatography [Silicycle C18 column; Mobile phase A: 0.1% formic acid in water; Mobile phase B: MeCN] to get tert-butyl (3S)-4-[1-(2,6-dioxo-3-piperidyl)-6-fluoro-3-methyl-2-oxo-benzimidazol-5-yl]-3-methyl-piperazine-1-carboxylate (8, 130 mg, 246.02 μmol, 29% yield, Formic acid salt) as a colorless solid.

LCMS (ES+): m/z 476.2 [M+H]⁺

### Step 6: 3-[6-fluoro-3-methyl-5-[(2S)-2-methylpiperazin-1-yl]-2-oxo-benzimidazol-1-yl]piperidine-2,6-dione (9)

Into a 25 mL single neck round-bottom flask containing a well-stirred solution of tert-butyl (3S)-4-[1-(2,6-dioxo-3-piperidyl)-6-fluoro-3-methyl-2-oxo-benzimidazol-5-yl]-3-methyl-piperazine-1-carboxylate (8, 125 mg, 236.56 μmol, Formic acid salt) in anhydrous DCM (2 mL) was added TFA (1.48 g, 12.98 mmol, 1 mL). After 2 h, the volatiles were removed under reduced pressure to obtain 3-[6-fluoro-3-methyl-5-[(2S)-2-methylpiperazin-1-yl]-2-oxo-benzimidazol-1-yl]piperidine-2,6-dione (9, 85 mg, 161.34 μmol, 68% yield, TFA salt) as brown solid.

LCMS (ES+): m/z 376.2 [M+H]⁺

### Step 7: tert-butyl 2-[(3S)-4-[1-(2,6-dioxo-3-piperidyl)-6-fluoro-3-methyl-2-oxo-benzimidazol-5-yl]-3-methyl-piperazin-1-yl]acetate (11)

Into a 25 mL single neck round-bottom flask containing a solution of 3-[6-fluoro-3-methyl-5-[(2S)-2-methylpiperazin-1-yl]-2-oxo-benzimidazol-1-yl]piperidine-2,6-dione (9, 80 mg, 151.85 μmol, TFA salt) in anhydrous DMF (2 mL) was added DIPEA (58.88 mg, 455.56 μmol, 79.35 μL) and cooled to 0° C. Then, a solution of tert-butyl 2-bromoacetate (10, 32.58 mg, 167.04 μmol, 24.50 μL) in anhydrous DMF (0.5 mL) was added dropwise and stirred the reaction mixture for 2 h at room temperature. The reaction mixture was diluted with ice-water and extracted with EtOAc (2×30 mL), dried over anhydrous Na₂SO₄, filtered and concentrated under reduced pressure. The crude compound was purified by reverse phase prep HPLC [Purification method: Column: XBridge C8 (19×150) mm, 5 micron; Mobile phase A: 0.1% formic acid in water; Mobile phase B: MeCN] to get tert-butyl 2-[(3S)-4-[1-(2,6-dioxo-3-piperidyl)-6-fluoro-3-methyl-2-oxo-benzimidazol-5-yl]-3-methyl-piperazin-1-yl]acetate (11, 70 mg, 129.57 μmol, 85% yield, Formic acid salt) as a colorless solid.

LCMS (ES+): m/z 490.2 [M+H]⁺

### Step 8: 2-[(3S)-4-[1-(2,6-dioxo-3-piperidyl)-6-fluoro-3-methyl-2-oxo-benzimidazol-5-yl]-3-methyl-piperazin-1-yl]acetic acid (12)

Into a 25 mL single neck round-bottom flask containing a well-stirred solution of tert-butyl 2-[(3S)-4-[1-(2,6-dioxo-3-piperidyl)-6-fluoro-3-methyl-2-oxo-benzimidazol-5-yl]-3-methyl-piperazin-1-yl]acetate (11, 65 mg, 120.31 μmol, Formic acid salt) in anhydrous DCM (1 mL) was added TFA (2.22 g, 19.47 mmol, 1.5 mL) at room temperature. The resulting solution was stirred at room temperature for 3 h. The solvent was removed under reduced pressure to get the 2-[(3S)-4-[1-(2,6-dioxo-3-piperidyl)-6-fluoro-3-methyl-2-oxo-benzimidazol-5-yl]-3-methyl-piperazin-1-yl]acetic acid (12, 60 mg, 109.07 μmol, 91% yield, TFA salt) as a colorless solid.

LCMS (ES+): m/z 434.0 [M+H]⁺

### 2-(1-(1-(2,6-bis(benzyloxy)pyridin-3-yl)-3-methyl-2-oxo-2,3-dihydro-1H-benzo[d]imidazol-5-yl)-4-hydroxypiperidin-4-yl)acetic acid (10)

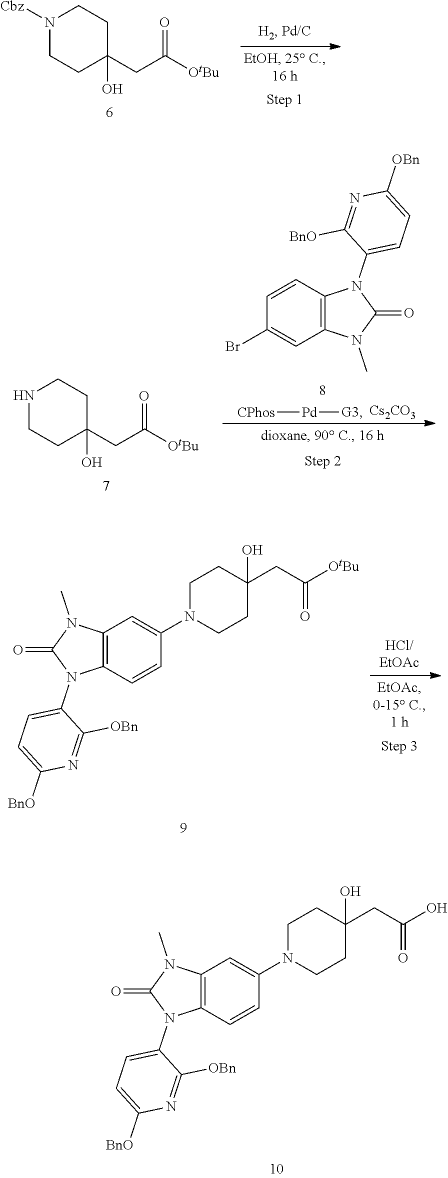

### Step 1: tert-butyl 2-(4-hydroxypiperidin-4-yl)acetate (7)

To a solution of benzyl 4-(2-(tert-butoxy)-2-oxoethyl)-4-hydroxypiperidine-1-carboxylate (6, 2 g, 5.72 mmol) in Ethanol (20 mL) was added Pd/C (200 mg, 10% purity). The mixture was stirred at 25° C. for 16 h under H₂ (15 Psi). The reaction mixture was concentrated under reduced pressure to afford tert-butyl 2-(4-hydroxypiperidin-4-yl)acetate (7, 1.2 g, 5.57 mmol, 97% yield), which was used to the next step without further purification.

¹H NMR (400 MHz, DMSO-d₆) δ (ppm)=4.43-4.22 (m, 1H), 2.81-2.72 (m, 2H), 2.66-2.58 (m, 2H), 2.26 (s, 2H), 1.56-1.44 (m, 4H), 1.40 (s, 9H)

### Step 2: tert-butyl 2-(1-(1-(2,6-bis(benzyloxy)pyridin-3-yl)-3-methyl-2-oxo-2,3-dihydro-1H-benzo[d]imidazol-5-yl)-4-hydroxypiperidin-4-yl)acetate (9)

To a solution of 1-(2,6-bis(benzyloxy)pyridin-3-yl)-5-bromo-3-methyl-1H-benzo[d]imidazol-2 (3H)-one (8, 500 mg, 968.27 μmol, 1 eq), tert-butyl 2-(4-hydroxypiperidin-4-yl)acetate (7, 312.69 mg, 1.45 mmol, 1.5 eq) in dioxane (2 mL) was added Cs₂CO₃ (788.70 mg, 2.42 mmol, 2.5 eq) and CPhos Pd G₃ (78.08 mg, 96.83 μmol, 0.1 eq). The mixture was stirred at 90° C. for 16 h under N₂. The reaction mixture was concentrated under reduced pressure. The residue purified by column chromatography (SiO₂, Petroleum ether:Ethyl acetate=1:0 to 1:1 to afford tert-butyl 2-(1-(1-(2,6-bis(benzyloxy)pyridin-3-yl)-3-methyl-2-oxo-2,3-dihydro-1H-benzo[d]imidazol-5-yl)-4-hydroxypiperidin-4-yl)acetate (9, 380 mg, 583.93 μmol, 60% yield) as a white solid.

LCMS (ESI): m/z 651.4 [M+H]⁺

### Step 3: 2-(1-(1-(2,6-bis(benzyloxy)pyridin-3-yl)-3-methyl-2-oxo-2,3-dihydro-1H-benzo[d]imidazol-5-yl)-4-hydroxypiperidin-4-yl)acetic acid (10)

To a solution of tert-butyl 2-(1-(1-(2,6-bis(benzyloxy)pyridin-3-yl)-3-methyl-2-oxo-2,3-dihydro-1H-benzo[d]imidazol-5-yl)-4-hydroxypiperidin-4-yl)acetate (9, 380 mg, 583.93 μmol) in EtOAc (3 mL) was added HCl/EtOAc (4 M, 4.75 mL) at 0° C. The mixture was stirred at 15° C. for 1 h. The reaction mixture was concentrated under reduced pressure. The residue was purified by prep-HPLC (flow: 25 mL/min; gradient: from 62%-32% water (0.1% TFA)-ACN; column: Phenomenex Luna C18 150×25 mm×10 um) to afford 2-(1-(1-(2,6-bis(benzyloxy)pyridin-3-yl)-3-methyl-2-oxo-2,3-dihydro-1H-benzo[d]imidazol-5-yl)-4-hydroxypiperidin-4-yl)acetic acid (10, 300 mg, 423.32 μmol, 73% yield) as a white solid.

LCMS (ESI): m/z 595.2 [M+H]⁺

### 2-((1-(1-(2,6-bis(benzyloxy)pyridin-3-yl)-3-methyl-2-oxo-2,3-dihydro-1H-benzo[d]imidazol-5-yl)piperidin-4-yl)methyl)cyclopropanecarboxylic acid (10)

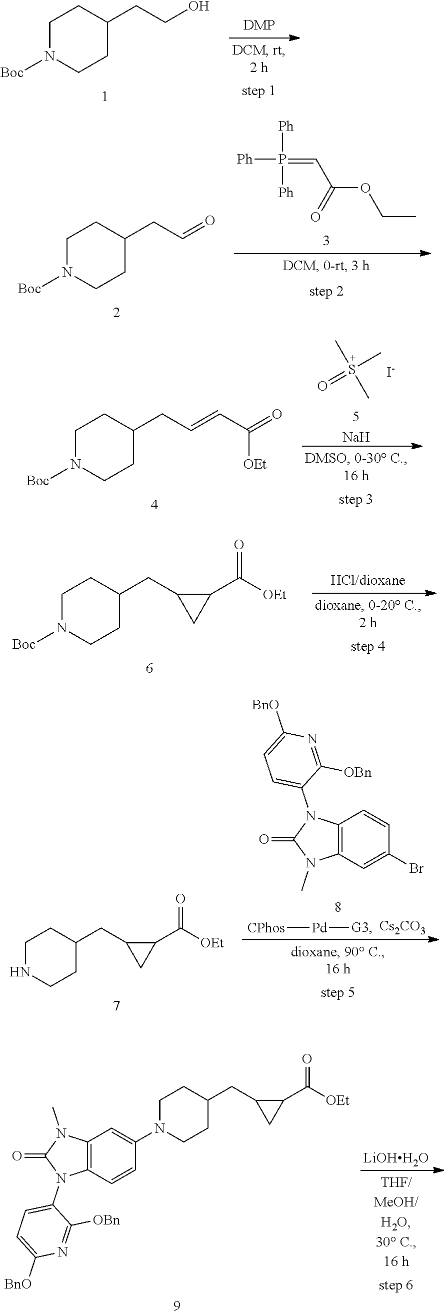

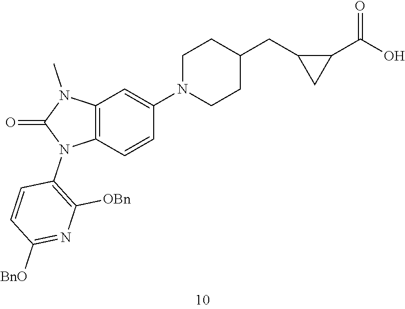 [SMILES: Cn1c(=O)n(-c2ccc(OCc3ccccc3)nc2OCc2ccccc2)c2ccc(N3CCC(CC4CC4C(=O)O)CC3)cc21]

### Step 1: tert-butyl 4-(2-oxoethyl)piperidine-1-carboxylate (2)

To a solution of tert-butyl 4-(2-hydroxyethyl)piperidine-1-carboxylate (1, 30 g, 130.82 mmol, 28.85 mL) in DCM (200 mL) was added Dess-Martin (66.59 g, 156.99 mmol, 1.2 q at 0° C. The mixture was stirred at 20° C. for 2 h. The mixture was concentrated under reduced pressure to give residue. The residue was purified by flash silica gel chromatography (120 mL/min, Eluent of 0-50% ethyl acetate/petroleum ether gradient, Column: ISCO; 100 g SepaFlash Silica Flash Column; Petroleum ether/Ethyl acetate=3/1) to afford tert-butyl 4-(2-oxoethyl)piperidine-1-carboxylate (2, 25 g, 109.99 mmol, 89% yield) as a yellow oil.

¹H NMR (400 MHz, d₆-DMSO) δ 9.68 (t, J=1.6 Hz, 1H), 3.90 (d, J=12.0 Hz, 2H), 2.72 (s, 2H), 2.37 (dd, J=1.6, 6.8 Hz, 2H), 2.05-1.93 (m, 1H), 1.66-1.55 (m, 2H), 1.39 (s, 9H), 1.05 (q, J=4.0, 12.0 Hz, 2H).

### Step 2: (E)-tert-butyl 4-(4-ethoxy-4-oxobut-2-en-1-yl)piperidine-1-carboxylate (4)

To a solution of tert-butyl 4-(2-oxoethyl)piperidine-1-carboxylate (2, 5 g, 22.00 mmol, 1 eq) in DCM (80 mL) was added ethyl 2-(triphenylphosphoranylidene)acetate (3, 13.79 g, 39.60 mmol, 1.8 eq) at 0° C. The mixture was stirred at 30° C. for 3 h. The reaction mixture was concentrated under reduced pressure to give a residue. The residue was purified by flash silica gel chromatography (60 mL/min, Eluent of 0-50% ethyl acetate/petroleum ether gradient, Column: ISCO; 50 g SepaFlash Silica Flash Column; Petroleum ether/Ethyl acetate=5/1) to afford (E)-tert-butyl 4-(4-ethoxy-4-oxobut-2-en-1-yl)piperidine-1-carboxylate (4, 6 g, 19.37 mmol, 88% yield) as a white solid.

LCMS (ESI): m/z 198.2 [M−Boc+H]⁺

¹H NMR (400 MHz, d₆-DMSO) δ 6.92-6.78 (m, 1H), 5.88 (d, J=15.6 Hz, 1H), 4.11 (q, J=6.8 Hz, 2H), 3.91 (d, J=11.6 Hz, 2H), 2.80-2.58 (m, 2H), 2.15 (t, J=6.4 Hz, 2H), 1.64-1.54 (m, 3H), 1.39 (s, 9H), 1.24-1.19 (m, 3H), 1.06-0.92 (m, 2H).

### Step 3: tert-butyl 4-((2-(ethoxycarbonyl)cyclopropyl)methyl)piperidine-1-carboxylate (6)

To a solution of NaH (504.34 mg, 12.61 mmol, 60% purity, 1.25 eq) in DMF (40 mL) at 0° C. was added Trimethylsulfoxonium Iodide (5, 2.89 g, 13.11 mmol, 1.3 eq) and stirred at 20° C. for 0.5 h. The mixture was added (E)-tert-butyl 4-(4-ethoxy-4-oxobut-2-en-1-yl)piperidine-1-carboxylate (4, 3 g, 10.09 mmol, 1 eq) and stirred at 30° C. for 5 h. The residue was purified by flash silica gel chromatography (75 mL/min, Eluent of 0-50% ethyl acetate/petroleum ether gradient, Column: ISCO; 20 g SepaFlash Silica Flash Column; Petroleum ether/Ethyl acetate=3/1) to afford tert-butyl 4-((2-(ethoxycarbonyl)cyclopropyl)methyl)piperidine-1-carboxylate (6, 400 mg, 1.05 mmol, 11% yield) as a white solid.

LCMS (ESI): m/z 212.2 [M−Boc+H]⁺

¹H NMR (400 MHz, d₆-DMSO) δ 4.04 (q, J=7.2 Hz, 2H), 3.92 (d, J=12.0 Hz, 2H), 2.76-2.61 (m, 2H), 1.64 (d, J=12.8 Hz, 2H), 1.56-1.46 (m, 1H), 1.39 (s, 10H), 1.28-1.20 (m, 3H), 1.18 (t, J=6.8 Hz, 3H), 1.06-0.96 (m, 3H), 0.77-0.69 (m, 1H).

### Step 4: ethyl 2-(piperidin-4-ylmethyl)cyclopropanecarboxylate (7)

To a solution of tert-butyl 4-((2-(ethoxycarbonyl)cyclopropyl)methyl)piperidine-1-carboxylate (6, 600 mg, 1.93 mmol, 1 eq) in dioxane (2 mL) was added HCl/dioxane (4 M, 10 mL) at 0° C. The mixture was stirred at 20° C. for 1 h. The reaction mixture was concentrated under reduced pressure to afford ethyl 2-(piperidin-4-ylmethyl)cyclopropanecarboxylate (7, 510 mg, 1.85 mmol, 96% yield, HCl salt) as a white solid. The crude product was used to next step without purification.

LCMS (ESI): m/z 211.8 [M+H]⁺

¹H NMR (400 MHz, d₆-DMSO) δ 4.11-3.98 (m, 2H), 3.21 (d, J=12.0 Hz, 2H), 2.88-2.73 (m, 2H), 1.79 (d, J=13.2 Hz, 2H), 1.70-1.56 (m, 1H), 1.44-1.16 (m, 9H), 1.01 (d, J=4.0, 8.0 Hz, 1H), 0.81-0.71 (m, 1H).

### Step 5: ethyl 2-((1-(1-(2,6-bis(benzyloxy)pyridin-3-yl)-3-methyl-2-oxo-2,3-dihydro-1H-benzo[d]imidazol-5-yl)piperidin-4-yl)methyl)cyclopropanecarboxylate (9)

A mixture of ethyl 2-(piperidin-4-ylmethyl)cyclopropanecarboxylate (7, 470 mg, 1.90 mmol, HCl salt, 2 eq) and 1-(2,6-bis(benzyloxy)pyridin-3-yl)-5-bromo-3-methyl-1H-benzo[d]imidazol-2 (3H)-one (8, 489.79 mg, 948.50 μmol, 1 eq) in dioxane (10 mL) were added Cphos Pd G3 (76.48 mg, 94.85 μmol, 0.1 eq) and CS₂CO₃ (927.11 mg, 2.85 mmol, 3 eq). The mixture was degassed and purged with N₂ 3 times, and then the mixture was stirred at 90° C. for 16 hr under N₂ atmosphere. The reaction mixture was filtered and the filtrate was concentrated under reduced pressure to give residue. The residue was purified by flash silica gel chromatography (60 mL/min, Eluent of 0-50% ethyl acetate/petroleum ether gradient, Column: ISCO; 5 g SepaFlash Silica Flash Column; Petroleum ether/Ethyl acetate=2/1) to afford ethyl 2-((1-(1-(2,6-bis(benzyloxy)pyridin-3-yl)-3-methyl-2-oxo-2,3-dihydro-1H-benzo[d]imidazol-5-yl)piperidin-4-yl)methyl)cyclopropanecarboxylate (9, 390 mg, 578.87 μmol, 61% yield) as a yellow solid.

LCMS (ESI): m/z 647.3 [M+H]⁺

¹H NMR (400 MHz, d₆-DMSO) δ 7.77 (d, J=8.0 Hz, 1H), 7.47-7.42 (m, 2H), 7.41-7.34 (m, 3H), 7.30-7.24 (m, 5H), 6.87-6.83 (m, 1H), 6.62-6.56 (m, 2H), 6.55-6.51 (m, 1H), 5.41-5.32 (m, 4H), 4.06-4.00 (m, 2H), 3.59 (dd, J=2.4, 8.0 Hz, 2H), 3.33 (s, 3H), 2.61 (t, J=11.6 Hz, 2H), 1.83-1.74 (m, 2H), 1.53-1.41 (m, 2H), 1.36-1.24 (m, 5H), 1.18-1.13 (m, 3H), 1.07-1.00 (m, 1H), 0.81-0.73 (m, 1H).

### Step 6: 2-((1-(1-(2,6-bis(benzyloxy)pyridin-3-yl)-3-methyl-2-oxo-2,3-dihydro-1H-benzo[d]imidazol-5-yl)piperidin-4-yl)methyl)cyclopropanecarboxylic acid (10)

To a solution of ethyl 2-((1-(1-(2,6-bis(benzyloxy)pyridin-3-yl)-3-methyl-2-oxo-2,3-dihydro-1H-benzo[d]imidazol-5-yl)piperidin-4-yl)methyl)cyclopropanecarboxylate (9, 390 mg, 602.99 μmol, 1 eq) in MeOH (3 mL), H₂O (3 mL) and THF (3 mL) was added LiOH H₂O (126.52 mg, 3.01 mmol, 5 eq). The mixture was stirred at 30° C. for 16 h. The reaction mixture was adjusted pH to 5 with 1 N HCl aqueous. Then the reaction mixture was diluted with water (10 mL) and extracted with ethyl acetate (20 mL×2). The combined organic layers were washed with brine (30 mL), dried over Na₂SO₄, filtered and concentrated under reduced pressure to afford 2-((1-(1-(2,6-bis(benzyloxy)pyridin-3-yl)-3-methyl-2-oxo-2,3-dihydro-1H-benzo[d]imidazol-5-yl)piperidin-4-yl)methyl)cyclopropanecarboxylic acid (10, 370 mg, 580.07 μmol, 96% yield) as an off-white solid. The crude product was used to next step without purification.

LCMS (ESI): m/z 619.1 [M+H]⁺

¹H NMR (400 MHz, d₆-DMSO) δ 12.16-11.97 (m, 1H), 7.81 (d, J=8.0 Hz, 2H), 7.47-7.44 (m, 2H), 7.42-7.35 (m, 3H), 7.34-7.20 (m, 6H), 6.97-6.73 (m, 1H), 6.63 (d, J=8.0 Hz, 1H), 5.42-5.35 (m, 4H), 3.57 (d, J=7.2 Hz, 3H), 3.41 (s, 3H), 1.92 (s, 2H), 1.87-1.72 (m, 2H), 1.42-1.21 (m, 6H), 1.05-0.99 (m, 1H), 0.74 (s, 1H).

### 2-(4-(1-(2,6-bis(benzyloxy)pyridin-3-yl)-3-methyl-2-oxo-2,3-dihydro-1H-benzo[d]imidazol-5-yl)-3-methoxyphenyl)acetic acid (4)

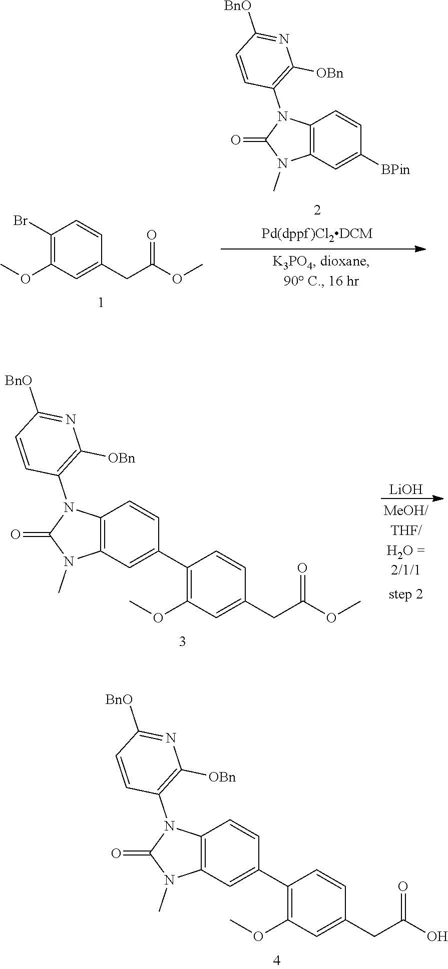

### Step 1: methyl 2-(4-(1-(2,6-bis(benzyloxy)pyridin-3-yl)-3-methyl-2-oxo-2,3-dihydro-1H-benzo[d]imidazol-5-yl)-3-methoxyphenyl)acetate (3)

A mixture of methyl 2-(4-bromo-3-methoxy phenyl)acetate (1, 200 mg, 771.91 μmol) and 1-(2,6-bis(benzyloxy)pyridin-3-yl)-3-methyl-5-(4,4,5,5-tetramethyl-1,3,2-dioxaborolan-2-yl)-1H-benzo[d]imidazol-2 (3H)-one (2, 395.40 mg, 701.74 μmol) in dioxane (10 mL) was added CsF (185.53 mg, 2.81 mmol) and cyclopentyl(diphenyl)phosphane; dichloromethane; dichloropalladium; iron (114.61 mg, 140.35 μmol) under N₂ atmosphere. The reaction mixture was stirred at 90° C. for 16 hr under N₂ atmosphere. The reaction mixture was concentrated to afford residue. The residue was purified by flash column (20 g silica gel column, 20%-50% EA in PE). The eluent was concentrated to afford methyl 2-(4-(1-(2,6-bis(benzyloxy)pyridin-3-yl)-3-methyl-2-oxo-2,3-dihydro-1H-benzo[d]imidazol-5-yl)-3-methoxyphenyl)acetate (3, 96 mg, 143.45 μmol, 20% yield) as yellow oil.

LCMS (ESI): m/z 615.9 [M+H]⁺

### Step 2: 2-(4-(1-(2,6-bis(benzyloxy)pyridin-3-yl)-3-methyl-2-oxo-2,3-dihydro-1H-benzo[d]imidazol-5-yl)-3-methoxyphenyl)acetic acid (4)

To a solution of methyl 2-[4-[1-(2,6-dibenzyloxy-3-pyridyl)-3-methyl-2-oxo-benzimidazol-5-yl]-3-methoxy-phenyl]acetate (3, 96 mg, 155.93 μmol) in THF (0.5 mL) and Methanol (1 mL) was added a solution of lithium; hydroxide; hydrate (32.72 mg, 779.63 μmol, 21.67 μL) in Water (0.5 mL). The mixture was stirred at 20° C. for 2 h. The reaction mixture was poured into water (5 mL) and adjusted pH=6 with 0.5 M HCl aqueous. The mixture was extracted by EA (3 mL×3), dried by anhydrous Na₂SO₄, filtered and concentrated to afford 2-[4-[1-(2,6-dibenzyloxy-3-pyridyl)-3-methyl-2-oxo-benzimidazol-5-yl]-3-methoxy-phenyl]acetic acid (4, 90 mg, 146.60 μmol, 94% yield) as white solid.

LCMS (ESI): m/z 602.2 [M+H]⁺

### 2-(4-(1-(2,6-bis(benzyloxy)pyridin-3-yl)-3-methyl-2-oxo-2,3-dihydro-1H-benzo[d]imidazol-5-yl)-3,5-dimethyl-1H-pyrazol-1-yl)acetic acid (6)

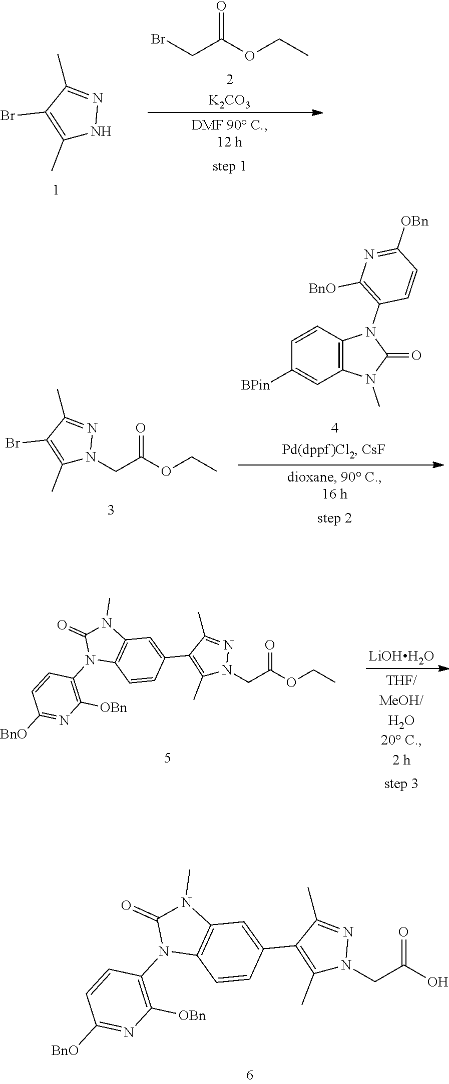

### Step 1: ethyl 2-(4-bromo-3,5-dimethyl-1H-pyrazol-1-yl)acetate (3)

To a mixture of 4-bromo-3,5-dimethyl-1H-pyrazole (1, 1 g, 5.71 mmol, 1 eq) and K₂CO₃ (1.58 g, 11.43 mmol, 2 eq) in DMF (10 mL) was added ethyl 2-bromoacetate (2, 1.05 g, 6.28 mmol, 695.07 μL, 1.1 eq). The mixture was stirred at 90° C. for 12 h. The reaction was diluted with H₂O (20 mL) and extracted with ethyl acetate (20 mL×2). The combined organic layers were washed with brine (20 mL×3), dried over Na₂SO₄, filtered and concentrated under vacuum. The residue was concentrated under vacuum and purified by flash silica gel chromatography (flow: 30 mL/min; gradient: 0-20% ethyl acetate in petroleum ether; ISCO®; 20 g SepaFlash® Silica Flash Column; ethyl acetate/petroleum ether=3/1) to afford ethyl 2-(4-bromo-3,5-dimethyl-1H-pyrazol-1-yl)acetate (3, 1.4 g, 5.36 mmol, 94% yield) as a yellow solid.

¹H NMR (400 MHz, DMSO-d₆) δ=4.99 (s, 2H), 4.16 (q, J=7.2 Hz, 2H), 2.21-2.04 (m, 6H), 1.21 (t, J=7.2 Hz, 3H)

### Step 2: ethyl 2-(4-(1-(2,6-bis(benzyloxy)pyridin-3-yl)-3-methyl-2-oxo-2,3-dihydro-1H-benzo[d]imidazol-5-yl)-3,5-dimethyl-1H-pyrazol-1-yl)acetate (5)

Into at 40 mL sealed tube reactor containing a well-stirred solution of 1-(2,6-bis(benzyloxy)pyridin-3-yl)-3-methyl-5-(4,4,5,5-tetramethyl-1,3,2-dioxaborolan-2-yl)-1H-benzo[d]imidazol-2 (3H)-one (4, 784.68 mg, 1.39 mmol, 1 eq) and ethyl 2-(4-bromo-3,5-dimethyl-1H-pyrazol-1-yl)acetate (3, 400 mg, 1.53 mmol, 1.1 eq) in anhydrous dioxane (8 mL) was added CsF (634.62 mg, 4.18 mmol, 3 eq) at 20° C. under nitrogen atmosphere and followed by Pd(dppf)Cl₂ (50.95 mg, 69.53 μmol, 0.05 eq). The resulting mixture was degassed by bubbling nitrogen gas into the reaction mixture for 5 mins and heated to 90° C. for 16 h. The reaction mixture was concentrated under reduced pressure and diluted with water (20 mL) and extracted with EA (10 mL*3). The combined organic layers were washed with brine (10 mL*2), dried over Na₂SO₄, filtered and concentrated under reduced pressure to give a residue. The residue was purified (flow: 35 mL/min; Eluent of 0-40% Ethylacetate/Petroleum ether gradient; ISCO®; 20 g SepaFlash® Silica Flash Column; Ethylacetate/Petroleum ether=1/1) to afford ethyl 2-(4-(1-(2,6-bis(benzyloxy)pyridin-3-yl)-3-methyl-2-oxo-2,3-dihydro-1H-benzo[d]imidazol-5-yl)-3,5-dimethyl-1H-pyrazol-1-yl)acetate (5, 700 mg, 1.11 mmol, 80% yield) as a yellow oil.

LCMS (ESI): m/z 618.1[M+H]⁺

### Step 3: 2-(4-(1-(2,6-bis(benzyloxy)pyridin-3-yl)-3-methyl-2-oxo-2,3-dihydro-1H-benzo[d]imidazol-5-yl)-3,5-dimethyl-1H-pyrazol-1-yl)acetic acid (6)

To a solution of ethyl 2-(4-(1-(2,6-bis(benzyloxy)pyridin-3-yl)-3-methyl-2-oxo-2,3-dihydro-1H-benzo[d]imidazol-5-yl)-3,5-dimethyl-1H-pyrazol-1-yl)acetate (490 mg, 569.57 μmol, 1 eq) in H₂O (5 mL) and THF (5 mL) and MeOH (5 mL) was added LiOH H₂O (119.51 mg, 2.85 mmol, 5 eg) at 20° C. and the mixture was stirred at 20° C. for 2 h. The reaction mixture was adjusted pH to 6 with 1N HCl aqueous. The mixture was diluted with H₂O (20 mL) and extracted with ethyl acetate (40 mL×2). The combined organic layers were washed with brine (50 mL), dried over Na₂SO₄, filtered and concentrated under vacuum. The residue was purified by flash silica gel chromatography (flow: 36 mL/min; gradient: 0-70% ethyl acetate in petroleum ether; 10 g SepaFlash® Silica Flash Column; ethyl acetate/petroleum=1/1) to afford 2-(4-(1-(2,6-bis(benzyloxy)pyridin-3-yl)-3-methyl-2-oxo-2,3-dihydro-1H-benzo[d]imidazol-5-yl)-3,5-dimethyl-1H-pyrazol-1-yl)acetic acid (6, 200 mg, 339.19 μmol, 60% yield) as a yellow solid.

LCMS (ESI): m/z 590.5 [M+H]⁺

### 2-(4-((1-(2,6-bis(benzyloxy)pyridin-3-yl)-3-methyl-2-oxo-2,3-dihydro-1H-benzo[d]imidazol-5-yl)amino)-3-fluorophenyl)acetic acid (3)

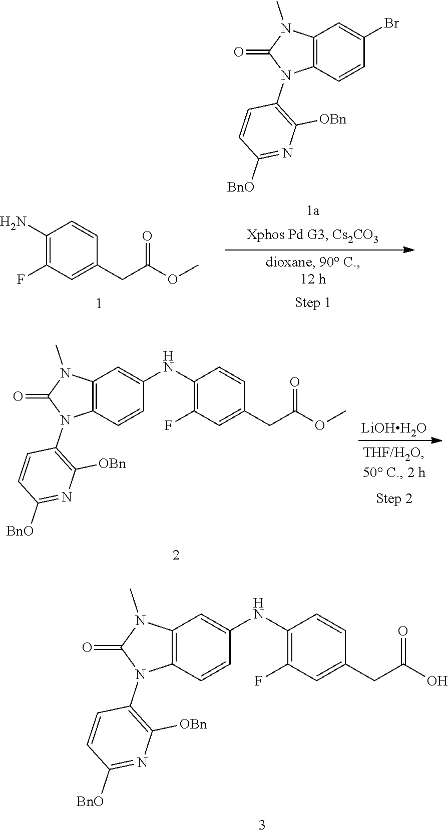

### Step 1: 2-(4-((1-(2,6-bis(benzyloxy)pyridin-3-yl)-3-methyl-2-oxo-2,3-dihydro-1H-benzo[d]imidazol-5-yl)amino)-3-fluorophenyl)acetate (2)

To a solution of methyl 2-(4-amino-3-fluoro-phenyl)acetate (1, 339.22 mg, 1.85 mmol) and 5-bromo-1-(2,6-dibenzyloxy-3-pyridyl)-3-methyl-3a, 7a-dihydrobenzimidazol-2-one (1a, 800 mg, 1.54 mmol) in dioxane (10 mL) were added Xphos Pd G3 (130.62 mg, 154.32 μmol) and dicesium; carbonate (1.26 g, 3.86 mmol). The reaction mixture was stirred at 90° C. for 12 hrs under N₂. The reaction mixture was concentrated under reduced pressure to remove solvent. The residue was diluted with H₂O (15 mL) and extracted with EA (15 mL*3). The combined organic layers were washed with brine (20 mL*3), dried over [anhydrous Na₂SO₄], filtered and concentrated under reduced pressure. The residue was purified by flash silica gel chromatography (ISCO®; 40 g SepaFlash® Silica Flash Column, Eluent of 0-80% Ethylacetate/Petroleum ether gradient @40 mL/min), the elute was concentrated under reduced pressure to afford methyl 2-(4-((1-(2,6-bis(benzyloxy)pyridin-3-yl)-3-methyl-2-oxo-2,3-dihydro-1H-benzo[d]imidazol-5-yl)amino)-3-fluorophenyl)acetate (2, 700 mg, 1.09 μmol, 70% yield) as red oil.

LCMS (ESI): m/z 619.2 [M+H]⁺

¹H NMR (400 MHz, CDCl3) δ=7.55 (d, J=8.4 Hz, 1H), 7.39-7.24 (m, 5H), 7.21-7.15 (m, 5H), 7.03-6.92 (m, 2H), 6.82-6.80 (m, 1H), 6.77 (d, J=2.0 Hz, 1H), 6.73-6.71 (m, 1H), 6.54 (d, J=8.4 Hz, 1H), 6.44 (d, J=8.4 Hz, 1H), 5.66 (br s, 1H), 5.46-5.37 (m, 1H), 5.28 (s, 2H), 5.25-5.18 (m, 1H), 3.64 (s, 3H), 3.48 (s, 2H), 3.36 (s, 3H)

### Step 2: 2-(4-((1-(2,6-bis(benzyloxy)pyridin-3-yl)-3-methyl-2-oxo-2,3-dihydro-1H-benzo[d]imidazol-5-yl)amino)-3-fluorophenyl)acetic acid (3)

To a solution of methyl 2-[4-[[1-(2,6-dibenzyloxy-3-pyridyl)-3-methyl-2-oxo-3a, 7a-dihydrobenzimidazol-5-yl]amino]-3-fluoro-phenyl]acetate (2, 700 mg, 1.13 mmol) in H₂O (5 mL), MeOH (5 mL) and THF (5 mL) was added Lithium hydroxide, monohydrate (473.27 mg, 11.28 mmol, 313.43 uL). The mixture was stirred at 50° C. for 2 hrs. The reaction mixture was concentrated under reduced pressure to remove solvent. The residue was diluted with H₂O (20 mL) and extracted with EA (10 mL*3). The combined organic layers were dried over anhydrous Na₂SO₄, filtered and concentrated under reduced pressure. The residue was purified by flash silica gel chromatography (ISCO®; 20 g SepaFlash® Silica Flash Column, Eluent of 0-100% Ethylacetate/Petroleum ether gradient @ 30 mL/min), and the eluent was concentrated to give 2-[4-[[1-(2,6-dibenzyloxy-3-pyridyl)-3-methyl-2-oxo-3a, 7a-dihydrobenzimidazol-5-yl]amino]-3-fluoro-phenyl]acetic acid (3, 450 mg, 741.79 μmol, 66% yield) as a yellow solid.

LCMS (ES+): m/z 604.8 [M+H]⁺

### 5-(4-aminophenyl)-1-(2,6-bis(benzyloxy)pyridin-3-yl)-3-methyl-1H-benzo[d]imidazol-2 (3H)-one (3)

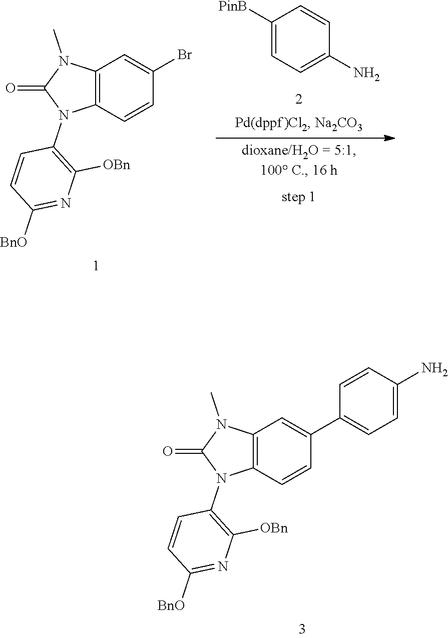

### Step 1: 5-(4-aminophenyl)-1-(2,6-bis(benzyloxy)pyridin-3-yl)-3-methyl-1H-benzo[d]imidazol-2 (3H)-one (3)

To a solution of 1-(2,6-bis(benzyloxy)pyridin-3-yl)-5-bromo-3-methyl-1H-benzo[d]imidazol-2 (3H)-one (1, 300 mg, 580.96 μmol) 4-(4,4,5,5-tetramethyl-1,3,2-dioxaborolan-2-yl) aniline (2, 152.74 mg, 697.15 μmol) disodium; carbonate (123.15 mg, 1.16 mmol, 48.68 μL) in Water (0.6 mL) dioxane (3 mL) was added cyclopentyl(diphenyl)phosphane; dichloropalladium; iron (42.51 mg, 58.10 μmol). The reaction was stirred at 110° C. for 2 h. The reaction mixture was filtered and the organic layer was concentrated under reduced (60 mL/min, Eluent of 0-100% ethyl acetate/petroleum ether) to obtained the 5-(4-aminophenyl)-1-(2,6-bis(benzyloxy)pyridin-3-yl)-3-methyl-1H-benzo[d]imidazol-2 (3H)-one (3, 280 mg, 476.73 μmol, 82% yield) as a yellow oil.

LCMS (ESI): m/z 529.3 [M+H]⁺

### 2-[1-[1-(2,6-dioxo-3-piperidyl)-6-fluoro-3-methyl-2-oxo-benzimidazol-5-yl]-4-hydroxy-4-piperidyl]acetic acid (5)

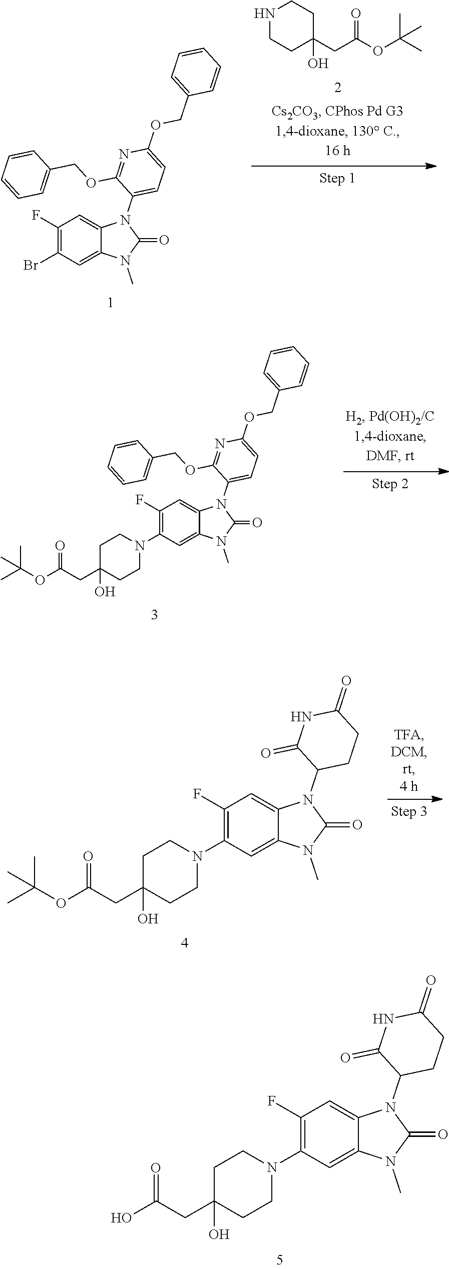 [SMILES: Cn1c(=O)n(-c2ccc(OCc3ccccc3)nc2OCc2ccccc2)c2cc(F)c(Br)cc21 ; CC(C)(C)OC(=O)CC1(O)CCNCC1 ; Cn1c(=O)n(-c2ccc(OCc3ccccc3)nc2OCc2ccccc2)c2cc(F)c(N3CCC(O)(CC(=O)OC(C)(C)C)CC3)cc21 ; Cn1c(=O)n(C2CCC(=O)NC2=O)c2cc(F)c(N3CCC(O)(CC(=O)OC(C)(C)C)CC3)cc21 ; Cn1c(=O)n(C2CCC(=O)NC2=O)c2cc(F)c(N3CCC(O)(CC(=O)O)CC3)cc21]

### Step 1: tert-butyl 2-[1-[1-(2,6-dibenzyloxy-3-pyridyl)-6-fluoro-3-methyl-2-oxo-benzimidazol-5-yl]-4-hydroxy-4-piperidyl]acetate (3)

Into a 50 mL pressure tube containing a well-stirred solution of 5-bromo-1-(2,6-dibenzyloxy-3-pyridyl)-6-fluoro-3-methyl-benzimidazol-2-one (1, 200 mg, 366.78 μmol) in anhydrous 1,4-dioxane (5 mL) were added tert-butyl 2-(4-hydroxy-4-piperidyl)acetate (343.32 mg, 733.57 μmol), CPhos Pd G3 (29.58 mg, 36.68 μmol) and cesium carbonate (358.52 mg, 1.10 mmol) at ambient temperature under nitrogen atmosphere. The resulting mixture was degassed by bubbling nitrogen gas for 10 min and stirred at 130° C. After 16 h, the reaction mixture was filtered through Celite and the filtrate was concentrated under reduced pressure. The crude product was purified by flash silica-gel column chromatography (5-50% EtOAc in pet ether) to afford tert-butyl 2-[1-[1-(2,6-dibenzyloxy-3-pyridyl)-6-fluoro-3-methyl-2-oxo-benzimidazol-5-yl]-4-hydroxy-4-piperidyl]acetate (3, 70 mg, 98.39 μmol, 27% yield)

LCMS (ES+): m/z 669.2 [M+H]⁺

### Step 2: tert-butyl 2-[1-[1-(2,6-dioxo-3-piperidyl)-6-fluoro-3-methyl-2-oxo-benzimidazol-5-yl]-4-hydroxy-4-piperidyl]acetate (4)

Into a 20 mL single-neck round-bottom flask containing a well-stirred solution of tert-butyl 2-[1-[1-(2,6-dibenzyloxy-3-pyridyl)-6-fluoro-3-methyl-2-oxo-benzimidazol-5-yl]-4-hydroxy-4-piperidyl]acetate (3, 200 mg, 281.12 μmol) in anhydrous 1,4-dioxane (5 mL) was added 20 wt. % palladium hydroxide on carbon (200 mg, 284.82 μmol, 20% purity) under nitrogen atmosphere. Then, the resulting suspension was stirred under hydrogen atmosphere (bladder) at room temperature. After 16 h, the reaction mixture was filtered and the filtrate was concentrated under reduced pressure. The residue was triturated with MTBE (10 mL), filtered and dried to afford tert-butyl 2-[1-[1-(2,6-dioxo-3-piperidyl)-6-fluoro-3-methyl-2-oxo-benzimidazol-5-yl]-4-hydroxy-4-piperidyl]acetate (4, 140 mg, 234.04 μmol, 83% yield) as a colorless solid.

UPLC-MS (ES+): m/z 491.7 [M+H]⁺

### Step 3: 2-[1-[1-(2,6-dioxo-3-piperidyl)-6-fluoro-3-methyl-2-oxo-benzimidazol-5-yl]-4-hydroxy-4-piperidyl]acetic acid (5)

Into a 20 mL single neck round-bottom flask containing a well-stirred solution of tert-butyl 2-[1-[1-(2,6-dioxo-3-piperidyl)-6-fluoro-3-methyl-2-oxo-benzimidazol-5-yl]-4-hydroxy-4-piperidyl]acetate (4, 160 mg, 319.66 μmol) in anhydrous DCM (5 mL) was added TFA (2.96 g, 25.96 mmol, 2.0 mL) at ambient temperature. The resulting solution was stirred at ambient temperature for 4 h. The reaction mixture was concentrated under reduced pressure to dryness and the residue was purified by reverse phase prep HPLC [Purification method: Column: Xbridge C18 (20×150) mm; Mobile phase A: 0.1% TFA in water; Mobile phase B: MeCN] to afford 2-[1-[1-(2,6-dioxo-3-piperidyl)-6-fluoro-3-methyl-2-oxo-benzimidazol-5-yl]-4-hydroxy-4-piperidyl]acetic acid (5, 70 mg, 126.60 μmol, 40% yield, TFA salt) as a white solid.

LCMS (ES+): m/z 435.2 [M+H]⁺

### 2-[4-[3-(2,6-dioxo-3-piperidyl)-1-methyl-2-oxo-8,9-dihydro-7H-imidazo[4,5-f]quinolin-6-yl]-1-piperidyl]acetic acid (7)

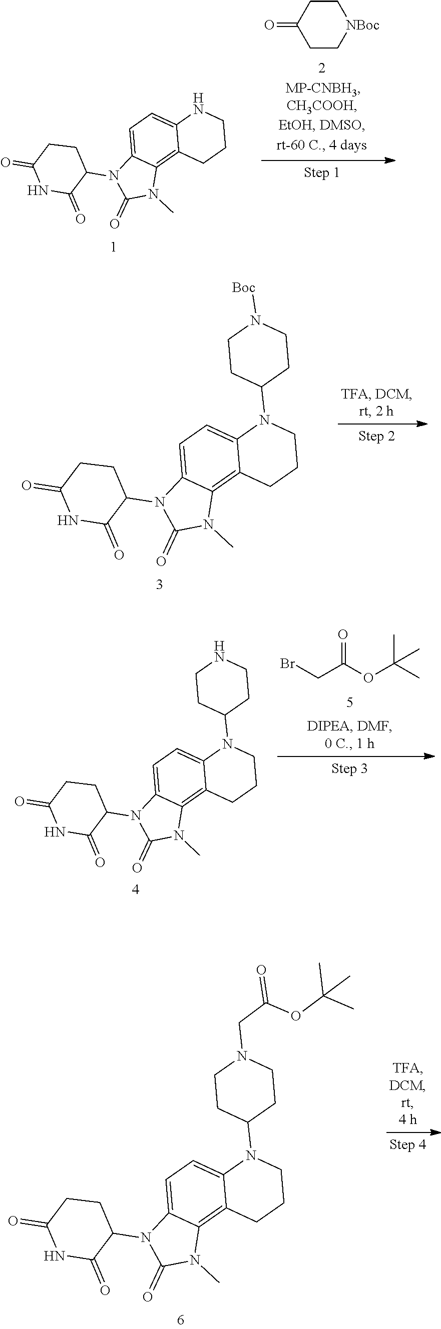

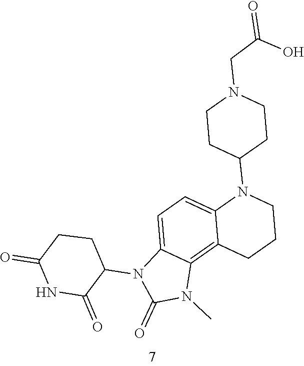 [SMILES: Cn1c(=O)n(C2CCC(=O)NC2=O)c2ccc3c(c21)CCCN3C1CCN(CC(=O)O)CC1]

### Step 1: tert-butyl 4-[3-(2,6-dioxo-3-piperidyl)-1-methyl-2-oxo-8,9-dihydro-7H-imidazo[4,5-f]quinolin-6-yl]piperidine-1-carboxylate (3)

Into a 50 mL single-neck round-bottom flask containing a well-stirred solution of 3-(1-methyl-2-oxo-6,7,8,9-tetrahydroimidazo[4,5-f]quinolin-3-yl)piperidine-2,6-dione (1, 130 mg, 347.40 μmol) and tert-butyl 4-oxopiperidine-1-carboxylate (2, 207.65 mg, 1.04 mmol) in anhydrous DMSO (4 mL) was added acetic acid (525.00 mg, 8.74 mmol, 0.5 mL) at room temperature. The contents were stirred at room temperature for 2 h. Then MP-cyano borohydride (0.35 g, 694.79 μmol, 2 mmol/g) was added and stirring continued for 24 h at room temperature and at to 60° C. for 72 h. The reaction mixture was passed through a sintered funnel, filtrate was concentrated under reduced pressure to get the crude compound, which was purified by reverse phase column chromatography [Silicycle C18 column; Mobile phase A: 10 mM NH₄OAc in water; Mobile phase B: MeCN] to get tert-butyl 4-[3-(2,6-dioxo-3-piperidyl)-1-methyl-2-oxo-8,9-dihydro-7H-imidazo[4,5-f]quinolin-6-yl]piperidine-1-carboxylate (3, 80 mg, 154.75 μmol, 45% yield) as an off-white solid.

LCMS (ES+): m/z 498.2 [M+H]⁺

### Step 2: 3-[1-methyl-2-oxo-6-(4-piperidyl)-8,9-dihydro-7H-imidazo[4,5-f]quinolin-3-yl]piperidine-2,6-dione (4)

Into a 25 mL single-neck round-bottom flask containing a well-stirred solution of tert-butyl 4-[3-(2,6-dioxo-3-piperidyl)-1-methyl-2-oxo-8,9-dihydro-7H-imidazo[4,5-f]quinolin-6-yl]piperidine-1-carboxylate (3, 65 mg, 125.73 μmol) in anhydrous DCM (3 mL) was added TFA (808.45 mg, 7.09 mmol, 546.25 μL) at room temperature. The resulting solution was stirred at room temperature for 2 h. The solvent was removed to dryness and the crude compound was purified by reverse phase column chromatography [Silicycle C18 column; Mobile phase A: 0.1% TFA in water; Mobile phase B: MeCN] to get 3-[1-methyl-2-oxo-6-(4-piperidyl)-8,9-dihydro-7H-imidazo[4,5-f]quinolin-3-yl]piperidine-2,6-dione (4, 45 mg, 87.85 μmol, 70% yield, TFA salt) as an off-white solid.

LCMS (ES+): m/z 397.2 [M+H]⁺

### Step 3: tert-butyl 2-[4-[3-(2,6-dioxo-3-piperidyl)-1-methyl-2-oxo-8,9-dihydro-7H-imidazo[4,5-f]quinolin-6-yl]-1-piperidyl]acetate (6)

Into a 25 mL single-neck round-bottom flask containing well-stirred solution of 3-[1-methyl-2-oxo-6-(4-piperidyl)-8,9-dihydro-7H-imidazo[4,5-f]quinolin-3-yl]piperidine-2,6-dione (4, 60 mg, 114.76 μmol, TFA salt) in anhydrous DMF (1 mL) were added DIPEA (29.66 mg, 229.52 μmol, 39.98 μL) and tert-butyl 2-bromoacetate (5, 22.38 mg, 114.76 μmol, 16.83 μL) at 0° C. The reaction mixture was stirred at 0° C. for 1 h. The reaction mixture was quenched with water (15 mL) and extracted with EtOAc (2×15 mL). The combined organic layer was dried over anhydrous Na₂SO₄, filtered and concentrated under reduced pressure. The crude compound was purified by reverse phase column chromatography [Silicycle C18 column; Mobile phase A: 0.1% formic acid in water; Mobile phase B: MeCN] to get tert-butyl 2-[4-[3-(2,6-dioxo-3-piperidyl)-1-methyl-2-oxo-8,9-dihydro-7H-imidazo[4,5-f]quinolin-6-yl]-1-piperidyl]acetate (6, 25 mg, 44.35 μmol, 39% yield, Formic acid salt) as an off-white solid. LCMS (ES+): m/z 512.3 [M+H]⁺

### Step 4: 2-[4-[3-(2,6-dioxo-3-piperidyl)-1-methyl-2-oxo-8,9-dihydro-7H-imidazo[4,5-f]quinolin-6-yl]-1-piperidyl]acetic acid (7)

Into a 25 mL single-neck round-bottom flask containing a well-stirred solution of tert-butyl 2-[4-[3-(2,6-dioxo-3-piperidyl)-1-methyl-2-oxo-8,9-dihydro-7H-imidazo[4,5-f]quinolin-6-yl]-1-piperidyl]acetate (6, 100 mg, 161.11 μmol, Formic acid salt) in anhydrous DCM (2 mL) was added TFA (1.18 g, 10.38 mmol, 0.8 mL). After 4 h, the solvent was removed under reduced pressure. The crude compound was purified by reverse phase column chromatography [Silicycle C18 column; Mobile phase A: 0.1% TFA in water; Mobile phase B: MeCN] to get 2-[4-[3-(2,6-dioxo-3-piperidyl)-1-methyl-2-oxo-8,9-dihydro-7H-imidazo[4,5-f]quinolin-6-yl]-1-piperidyl]acetic acid (7, 90 mg, 147.14 μmol, 91% yield, TFA salt) as off white solid.

LCMS (ES+): m/z 455.2 [M+H]⁺

### 2-[4-[1-(2,6-dioxo-3-piperidyl)-3-methyl-2-oxo-benzimidazol-5-yl]oxyphenyl]acetic acid (5)

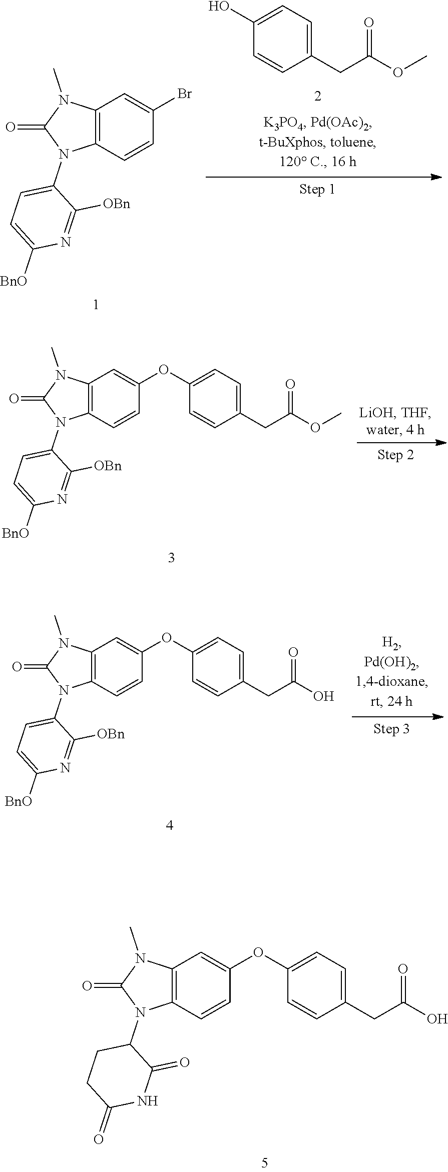 [SMILES: Cn1c(=O)n(-c2ccc(OCc3ccccc3)nc2OCc2ccccc2)c2ccc(Br)cc21 ; COC(=O)Cc1ccc(O)cc1 ; COC(=O)Cc1ccc(Oc2ccc3c(c2)n(C)c(=O)n3-c2ccc(OCc3ccccc3)nc2OCc2ccccc2)cc1 ; Cn1c(=O)n(-c2ccc(OCc3ccccc3)nc2OCc2ccccc2)c2ccc(Oc3ccc(CC(=O)O)cc3)cc21 ; Cn1c(=O)n(C2CCC(=O)NC2=O)c2ccc(Oc3ccc(CC(=O)O)cc3)cc21]

### Step 1: methyl 2-[4-[1-(2,6-dibenzyloxy-3-pyridyl)-3-methyl-2-oxo-benzimidazol-5-yl]oxyphenyl]acetate (3)

Into a 25 mL pressure tube containing a well-stirred solution of 5-bromo-1-(2,6-dibenzyloxy-3-pyridyl)-3-methyl-benzimidazol-2-one (1, 0.5 g, 957.04 μmol) and methyl 2-(4-hydroxyphenyl)acetate (2, 159.03 mg, 957.04 μmol) in anhydrous toluene (5 mL) was added anhydrous potassium phosphate tribasic (812.59 mg, 3.83 mmol) at room temperature. The reaction mixture was degassed by bubbling nitrogen gas for 10 min. Then palladium (II) acetate (64.46 mg, 287.11 μmol) and tBuXPhos (121.92 mg, 287.11 μmol) were added. The reaction mixture was stirred at 120° C. After 16 h, the reaction mixture was passed through Celite. The filtrate was concentrated under reduced pressure to get the crude compound, which was purified by flash silica gel (230-400 mesh) column chromatography (47% EtOAc in pet ether) to afford methyl 2-[4-[1-(2,6-dibenzyloxy-3-pyridyl)-3-methyl-2-oxo-benzimidazol-5-yl]oxyphenyl]acetate (3, 0.3 g, 415.61 μmol, 43% yield) as light brown sticky solid.

UPLC (ES+): m/z 602.8 [M+H]⁺

### Step 2: 2-[4-[1-(2,6-dibenzyloxy-3-pyridyl)-3-methyl-2-oxo-benzimidazol-5-yl]oxyphenyl]acetic acid (4)

Into a 50 mL single-neck round-bottom flask containing a well-stirred solution of methyl 2-[4-[1-(2,6-dibenzyloxy-3-pyridyl)-3-methyl-2-oxo-benzimidazol-5-yl]oxyphenyl]acetate (3, 0.3 g, 413.86 μmol) in THF (2 mL) and water (2 mL) was added lithium hydroxide monohydrate (86.84 mg, 2.07 mmol). After 4 h, the mixture was concentrated and acidified with 1.5 N aqueous HCl and extracted with EtOAc (3×30 mL). The combined organic layer was dried over anhydrous Na₂SO₄, filtered and filtrate was concentrated under reduced pressure to get 2-[4-[1-(2,6-dibenzyloxy-3-pyridyl)-3-methyl-2-oxo-benzimidazol-5-yl]oxyphenyl]acetic acid (4, 0.25 g, 361.63 μmol, 87% yield) as an off-white solid.

UPLC (ES+): m/z 589.0 [M+H]⁺

### Step 3: 2-[4-[1-(2,6-dioxo-3-piperidyl)-3-methyl-2-oxo-benzimidazol-5-yl]oxyphenyl]acetic acid (5)

Into a 25 mL single-neck round-bottom flask containing a well-stirred solution of 2-[4-[1-(2,6-dibenzyloxy-3-pyridyl)-3-methyl-2-oxo-benzimidazol-5-yl]oxyphenyl]acetic acid (4, 0.25 g, 361.63 μmol) in anhydrous 1,4-dioxane (3 mL) was added palladium hydroxide 20 wt. % on carbon (253.93 mg, 361.63 μmol, 20% purity) at room temperature. The suspension was stirred under hydrogen at room temperature. After 16 h, the reaction mixture was filtered through Celite bed. The filtrate was concentrated under reduced pressure and the crude compound was purified by reverse phase column chromatography [Silicycle C18 column; Mobile phase A: 0.1% formic acid in water; Mobile phase B: MeCN] to get 2-[4-[1-(2,6-dioxo-3-piperidyl)-3-methyl-2-oxo-benzimidazol-5-yl]oxyphenyl]acetic acid (5, 100 mg, 239.89 μmol, 66% yield) as an off-white solid.

LCMS (ES+): m/z 410.0 [M+H]⁺2-(3-(1-(2,6-bis(benzyloxy)pyridin-3-yl)-3-methyl-2-oxo-2,3-dihydro-1H-benzo[d]imidazol-5-yl)phenyl)acetic acid (2)

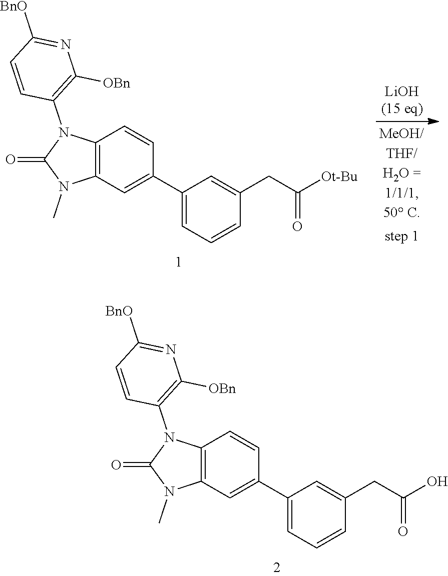 [SMILES: Cn1c(=O)n(-c2ccc(OCc3ccccc3)nc2OCc2ccccc2)c2ccc(-c3cccc(CC(=O)OC(C)(C)C)c3)cc21 ; Cn1c(=O)n(-c2ccc(OCc3ccccc3)nc2OCc2ccccc2)c2ccc(-c3cccc(CC(=O)O)c3)cc21]

### Step 1: 2-(3-(1-(2,6-bis(benzyloxy)pyridin-3-yl)-3-methyl-2-oxo-2,3-dihydro-1H-benzo[d]imidazol-5-yl)phenyl)acetic acid (2)

To a solution of methyl tert-butyl 2-(3-(1-(2,6-bis(benzyloxy)pyridin-3-yl)-3-methyl-2-oxo-2,3-dihydro-1H-benzo[d]imidazol-5-yl)phenyl)acetate (1, 300 mg, 477.91 μmol) in Water (3 mL) and THF (3 mL) was added LiOH·H₂O (300.83 mg, 7.17 mmol, 199.22 uL). The mixture was stirred at 50° C. for 16 h. The reaction mixture was concentrated under reduced pressure to remove THF. The mixture was washed with ethyl acetate (10 mL×2). The pH of the water phase was adjusted to 4 with aq·HCl (1M). The water phase was extracted with ethyl acetate (10 mL×2). The combined organic layers were dried over Na₂SO₄, filtered and concentrated under vacuum to afford 2-(3-(1-(2,6-bis(benzyloxy)pyridin-3-yl)-3-methyl-2-oxo-2,3-dihydro-1H-benzo[d]imidazol-5-yl)phenyl)acetic acid (2, 200 mg, 347.75 μmol, 73% yield) as a white solid. The crude product was used in the next step without further purification.

LCMS (ES+): m/z 572.2 [M+H]⁺

### 2-(4-(1-(2,6-bis(benzyloxy)pyridin-3-yl)-3-methyl-2-oxo-2,3-dihydro-1H-benzo[d]imidazol-5-yl)-2-methylphenyl)acetic acid (5)

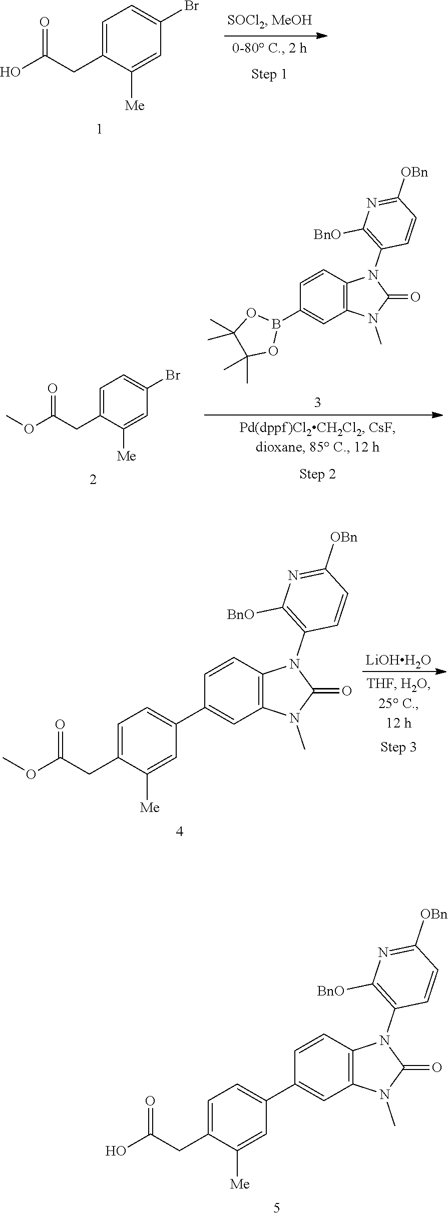

### Step 1: methyl 2-(4-bromo-2-methylphenyl)acetate (2)

To a solution of 2-(4-bromo-2-methylphenyl)acetic acid (1, 1.00 g, 4.37 mmol) in Methanol (10 mL) was added sulfurous dichloride (623.23 mg, 5.24 mmol, 380.02 μL) slowly at 0° C. and then the mixture was stirred at 80° C. for 2 hrs. The reaction mixture was concentrated under reduce pressure to afford methyl 2-(4-bromo-2-methylphenyl)acetate (2, 1.1 g, 3.98 mmol, 100% yield) as yellow oil.

LCMS (ES+): m/z 245.0 [M+H]⁺

¹H NMR (400 MHz, CDCl3) δ=7.26 (s, 1H), 7.24-7.17 (m, 1H), 6.99 (d, J=8.0 Hz, 1H), 3.62 (s, 3H), 3.52 (s, 2H), 2.21 (s, 3H)

### Step 2: methyl 2-(4-(1-(2,6-bis(benzyloxy)pyridin-3-yl)-3-methyl-2-oxo-2,3-dihydro-1H-benzo[d]imidazol-5-yl)-2-methylphenyl)acetate (4)

A mixture of 1-(2,6-bis(benzyloxy)pyridin-3-yl)-3-methyl-5-(4,4,5,5-tetramethyl-1,3,2-dioxaborolan-2-yl)-1H-benzo[d]imidazol-2 (3H)-one (3, 1.00 g, 1.77 mmol), methyl 2-(4-bromo-2-methylphenyl)acetate (2, 500.08 mg, 1.81 mmol), cesium fluoride (808.78 mg, 5.32 mmol, 196.31 μL) and cyclopentyl(diphenyl)phosphane; dichloromethane; dichloropalladium; iron (144.94 mg, 177.48 μmol) in dioxane (10 mL) was stirred at 85° C. for 12 hrs. The reaction mixture was poured into water 20 mL and extracted with EA 30 mL (10 mL*3). The organic layers were dried over anhydrous Na₂SO₄, filtered and concentrated to give a residue. The residue was purified by flash silica gel chromatography (ISCO®; 40 g SepaFlash® Silica Flash Column, Eluent of 0-100% EA/PE) and the eluent was concentrated under reduce pressure to afford methyl 2-(4-(1-(2,6-bis(benzyloxy)pyridin-3-yl)-3-methyl-2-oxo-2,3-dihydro-1H-benzo[d]imidazol-5-yl)-2-methylphenyl)acetate (4, 610 mg, 996.88 μmol, 56% yield) as yellow oil.

LCMS (ES+): m/z 600.2 [M+H]⁺

¹H NMR (400 MHz, DMSO-d6) δ=7.84 (d, J=8.0 Hz, 1H), 7.52 (d, J=1.2 Hz, 2H), 7.48-7.42 (m, 3H), 7.42-7.33 (m, 3H), 7.32-7.22 (m, 7H), 6.75 (d, J=8.4 Hz, 1H), 6.63 (d, J=8.4 Hz, 1H), 5.47-5.29 (m, 4H), 3.73 (s, 2H), 3.63 (s, 3H), 3.45 (s, 3H), 2.30 (s, 3H)

### Step 3: 2-(4-(1-(2,6-bis(benzyloxy)pyridin-3-yl)-3-methyl-2-oxo-2,3-dihydro-1H-benzo[d]imidazol-5-yl)-2-methylphenyl)acetic acid (5)

To a solution of methyl 2-(4-(1-(2,6-bis(benzyloxy)pyridin-3-yl)-3-methyl-2-oxo-2,3-dihydro-1H-benzo[d]imidazol-5-yl)-2-methylphenyl)acetate (4, 0.61 g, 996.88 μmol) in THF (7 mL) and Water (1.4 mL) was added lithium hydroxide hydrate (209.16 mg, 4.98 mmol, 138.52 μL) and the mixture was stirred at 25° C. for 12 hrs. The mixture was acidified with 1 M HCl to pH=4 and then extracted with EA 30 mL (10 mL*3). The combined organic layers were dried over anhydrous Na₂SO₄, filtered and concentrated to afford 2-(4-(1-(2,6-bis(benzyloxy)pyridin-3-yl)-3-methyl-2-oxo-2,3-dihydro-1H-benzo[d]imidazol-5-yl)-2-methylphenyl)acetic acid (5, 0.3 g, 512.25 μmol, 51% yield) as yellow oil.

LCMS (ES+): m/z 586.2 [M+H]⁺

¹H NMR (400 MHz, DMSO-d6) δ=12.32 (br d, J=0.8 Hz, 1H), 7.84 (d, J=8.4 Hz, 1H), 7.51 (s, 2H), 7.45 (br d, J=7.6 Hz, 3H), 7.42-7.33 (m, 3H), 7.32-7.21 (m, 7H), 6.75 (d, J=8.0 Hz, 1H), 6.63 (d, J=8.4 Hz, 1H), 5.45-5.33 (m, 4H), 3.65-3.60 (m, 2H), 3.45 (s, 3H), 2.31 (s, 3H)

### methyl 3-(3-(1-(2,6-bis(benzyloxy)pyridin-3-yl)-3-methyl-2-oxo-2,3-dihydro-1H-benzo[d]imidazol-5-yl)phenyl) propanoate (3)

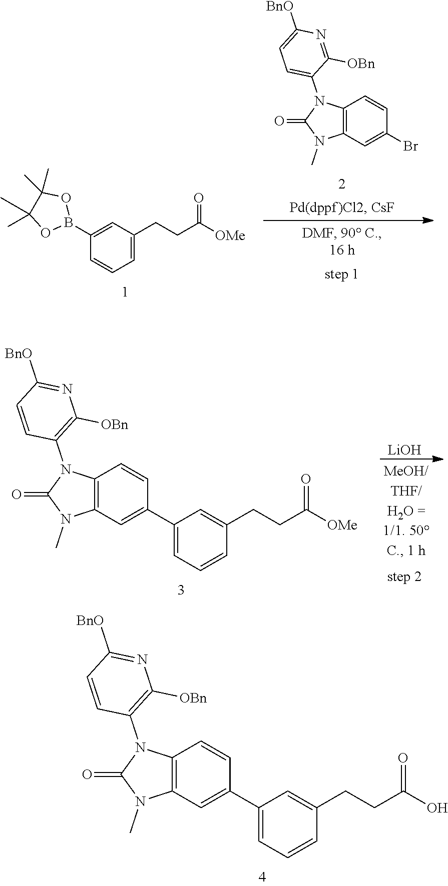 [SMILES: Cn1c(=O)n(-c2ccc(OCc3ccccc3)nc2OCc2ccccc2)c2ccc(Br)cc21 ; COC(=O)CCc1cccc(B2OC(C)(C)C(C)(C)O2)c1 ; COC(=O)CCc1cccc(-c2ccc3c(c2)n(C)c(=O)n3-c2ccc(OCc3ccccc3)nc2OCc2ccccc2)c1 ; "LiOH" ; Cn1c(=O)n(-c2ccc(OCc3ccccc3)nc2OCc2ccccc2)c2ccc(-c3cccc(CCC(=O)O)c3)cc21]

### Step 1: methyl 3-(3-(1-(2,6-bis(benzyloxy)pyridin-3-yl)-3-methyl-2-oxo-2,3-dihydro-1H-benzo[d]imidazol-5-yl)phenyl) propanoate (3)

To a solution of methyl 3-[3-(4,4,5,5-tetramethyl-1,3,2-dioxaborolan-2-yl)phenyl]propanoate (1, 337.15 mg, 1.16 mmol) and 1-(2,6-bis(benzyloxy)pyridin-3-yl)-5-bromo-3-methyl-1H-benzo[d]imidazol-2 (3H)-one (2, 0.5 g, 968.27 μmol) in DMF (15 mL) was added CsF (128.00 mg, 1.94 mmol) and cyclopentyl(diphenyl)phosphane; dichloropalladium; iron (35.42 mg, 48.41 μmol). The mixture was stirred at 90° C. under N₂ for 16 h. The mixture filtered and washed with ethyl acetate (50 mL). The filtrate was washed with brine (30 mL×2), dried over Na₂SO₄, filtered and concentrated under reduced pressure. The residue was purified by column chromatography (SiO₂, petroleum ether/ethyl acetate=100/0 to 1/1) to afford methyl 3-(3-(1-(2,6-bis(benzyloxy) pyridine-3-yl)-3-methyl-2-oxo-2,3-dihydro-1H-benzo[d]imidazol-5-yl)phenyl)propanoate (3, 200 mg, 323.51 μmol, 33% yield) as yellow oil.

LCMS (ESI): m/z 599.9 [M+H]⁺

### Step 2: 3-(3-(1-(2,6-bis(benzyloxy)pyridin-3-yl)-3-methyl-2-oxo-2,3-dihydro-1H-Benzo[d]imidazol-5-yl)phenyl)propanoic acid (4)

To a mixture of methyl 3-(3-(1-(2,6-bis(benzyloxy)pyridin-3-yl)-3-methyl-2-oxo-2,3-dihydro-1H-benzo[d]imidazol-5-yl)phenyl)propanoate (3, 0.2 g, 333.51 μmol) in THF (4 mL) Water (4 mL), Methanol (4 mL) was added LiOH (39.94 mg, 1.67 mmol). The mixture was stirred at 50° C. for 1 h. The reaction mixture was adjusted pH to 2 with 1N HCl aqueous and extracted with ethyl acetate (20 mL×2). The combined organic layers were washed with brine (30 mL×2), dried over Na₂SO₄, filtered and concentrated under reduced pressure to afford 3-(3-(1-(2,6-bis (benzyloxy) pyridin-3-yl)-3-methyl-2-oxo-2,3-dihydro-1H-benzo[d]imidazol-5-yl)phenyl) propanoic acid (4, 0.17 g, 282.73 μmol, 85% yield) as a white solid. The material was used into next step without further purification.

LCMS (ESI): m/z 586.2 [M+H]⁺

### 2-(4-((1-(2,6-bis(benzyloxy)pyridin-3-yl)-3-methyl-2-oxo-2,3-dihydro-1H-benzo[d]imidazol-5-yl)oxy)-3-methylphenyl)acetic acid (3)

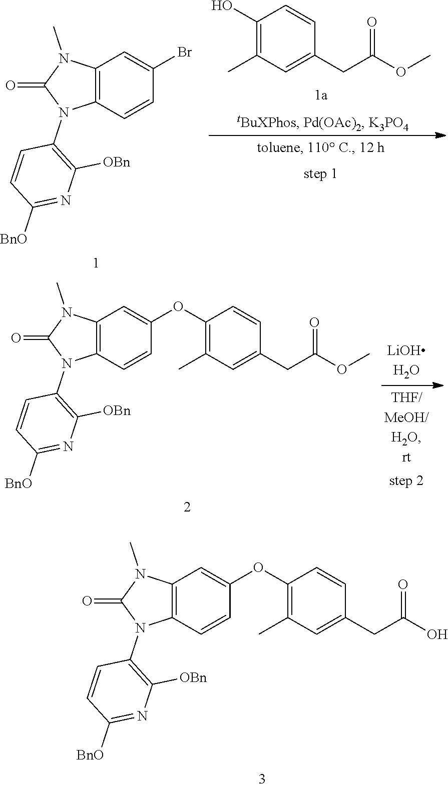

### Step 1: methyl 2-(4-((1-(2,6-bis(benzyloxy)pyridin-3-yl)-3-methyl-2-oxo-2,3-dihydro-1H-benzo[d]imidazol-5-yl)oxy)-3-methylphenyl)acetate (2)

To a solution of 1-(2,6-bis(benzyloxy)pyridin-3-yl)-5-bromo-3-methyl-1H-benzo[d]imidazol-2 (3H)-one (1, 500 mg, 968.27 μmol) and methyl 2-(4-hydroxy-3-methylphenyl)acetate (2, 348.96 mg, 1.94 mmol) in Toluene (7 mL) were added ditert-butyl-[2-[2,4,6-tri(propan-2-yl)phenyl]phenyl]phosphane (82.23 mg, 193.65 μmol), diacetoxypalladium (21.74 mg, 96.83 μmol) and Tripotassium phosphate (616.59 mg, 2.90 mmol). The reaction mixture was stirred at 110° C. for 16 hrs under N₂. The reaction mixture was concentrated under reduced pressure to remove solvent. The residue was diluted with H₂O (15 mL) and extracted with EA (15 mL*3). The combined organic layers were washed with brine (20 mL*3), dried over [anhydrous Na₂SO₄], filtered and concentrated under reduced pressure. The residue was purified by flash silica gel chromatography (ISCO®; 20 g SepaFlash® Silica Flash Column, Eluent of 0-60% Ethylacetate/Petroleum ether gradient @ 40 mL/min), the elute was concentrated under reduced pressure to get methyl 2-(4-((1-(2,6-bis(benzyloxy)pyridin-3-yl)-3-methyl-2-oxo-2,3-dihydro-1H-benzo[d]imidazol-5-yl)oxy)-3-methylphenyl)acetate (2, 220 mg, 344.84 μmol, 36% yield) as red oil.

LCMS (ESI): m/z 616.2 [M+H]⁺

### Step 2: 2-(4-((1-(2,6-bis(benzyloxy)pyridin-3-yl)-3-methyl-2-oxo-2,3-dihydro-1H-benzo[d]imidazol-5-yl)oxy)-3-methylphenyl)acetic acid (3)

To a solution of methyl 2-[4-[1-(2,6-dibenzyloxy-3-pyridyl)-3-methyl-2-oxo-benzimidazol-5-yl]oxy-3-methyl-phenyl]acetate (2, 0.22 g, 357.33 μmol) in Water (1.7 mL), Methanol (4.2 mL) and THF (4.2 mL) was added THF (4.2 mL). The mixture was stirred at 30° C. for 12 h. The reaction mixture was concentrated under reduced pressure to remove solvent. The residue was diluted with H₂O (5 mL) and extracted with EA (10 mL*3). The combined organic layers were dried over anhydrous Na₂SO₄, filtered and concentrated under reduced pressure. The residue was purified by flash silica gel chromatography (ISCO®; 20 g SepaFlash® Silica Flash Column, Eluent of 0-100% Ethylacetate/Petroleum ether gradient @ 30 mL/min), and the eluent was concentrated to give 2-(4-((1-(2,6-bis(benzyloxy)pyridin-3-yl)-3-methyl-2-oxo-2,3-dihydro-1H-benzo[d]imidazol-5-yl)oxy)-3-methylphenyl)acetic acid (3, 180 mg, 259.28 μmol, 73% yield) as a yellow solid.

LCMS (ESI): m/z 601.9 [M+H]⁺

### 6-(1-(2,6-bis(benzyloxy)pyridin-3-yl)-3-methyl-2-oxo-2,3-dihydro-1H-benzo[d]imidazol-5-yl)-1-methyl-1H-indole-3-carboxamide (6)

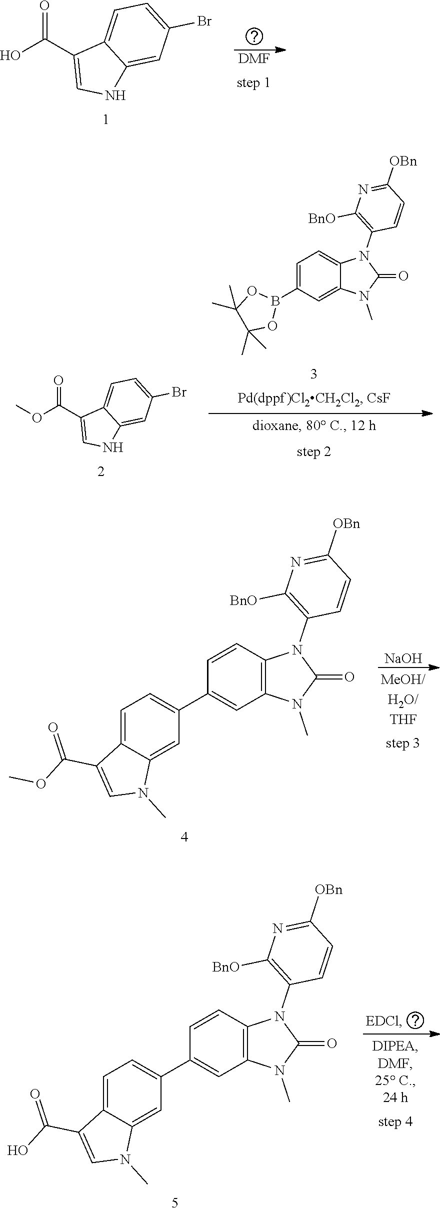

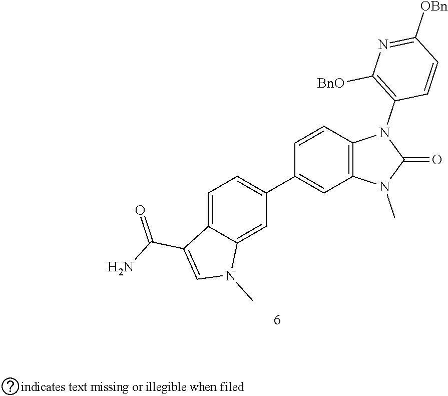 [SMILES: Cn1cc(C(N)=O)c2ccc(-c3ccc4c(c3)n(C)c(=O)n4-c3ccc(OCc4ccccc4)nc3OCc3ccccc3)cc21]

### Step 1: methyl 6-bromo-1-methyl-1H-indole-3-carboxylate (2)

To a solution of 6-bromo-1H-indole-3-carboxylic acid (1, 2 g, 8.33 mmol) in DMF (10 mL) was cooled to 0° C. Then NaH (957.70 mg, 24.99 mmol, 60% purity) was added to the mixture. The reaction was stirred at 0° C. for 0.5 h. Then iodomethane (7.10 g, 49.99 mmol, 3.11 mL) was added to the mixture. The reaction was stirred at 25° C. for 12 h. The mixture was quenched with saturated solution of NH₄Cl (100 mL), extracted with EtOAc (100 mL). The organic layer was washed with brine (100 mL) and then dried over Na₂SO₄, concentrated in vacuo to afford methyl 6-bromo-1-methyl-1H-indole-3-carboxylate (2, 2.0 g, 7.09 mmol, 85% yield) as a yellow solid.

LCMS (ESI): m/z 268.1/270.1 [M+H]⁺

### Step 2: methyl 6-(1-(2,6-bis(benzyloxy)pyridin-3-yl)-3-methyl-2-oxo-2,3-dihydro-1H-benzo[d]imidazol-5-yl)-1-methyl-1H-indole-3-carboxylate (4)

To a solution of methyl 6-bromo-1-methyl-1H-indole-3-carboxylate (2, 500 mg, 1.86 mmol) and 1-(2,6-bis(benzyloxy)pyridin-3-yl)-3-methyl-5-(4,4,5,5-tetramethyl-1,3,2-dioxaborolan-2-yl)-1H-benzo[d]imidazol-2 (3H)-one (3, 1.05 g, 1.86 mmol) in dioxane (5 mL) was added CsF (849.87 mg, 5.59 mmol, 0.206 mL) and cyclopentyl(diphenyl)phosphane; dichloromethane; dichloropalladium; iron (152.30 mg, 186.49 μmol). The mixture was stirred at 80° C. for 12 h. The reaction mixture was diluted with H₂O (20 mL) and extracted with Ethyl acetate (20 mL×3). The combined organic layers were washed with aqueous NaCl (20 mL), dried over Na₂SO₄, filtered and concentrated under reduced pressure. The residue was purified by column chromatography (SiO₂, Petroleum ether/Ethyl acetate=0% to 70%) to afford methyl 6-(1-(2,6-bis(benzyloxy)pyridin-3-yl)-3-methyl-2-oxo-2,3-dihydro-1H-benzo[d]imidazol-5-yl)-1-methyl-1H-indole-3-carboxylate (4, 700 mg, 1.08 mmol, 58% yield) as a yellow solid.

LCMS (ESI): m/z 625.4 [M+H]⁺

### Step 3: 6-(1-(2,6-bis(benzyloxy)pyridin-3-yl)-3-methyl-2-oxo-2,3-dihydro-1H-benzo[d]imidazol-5-yl)-1-methyl-1H-indole-3-carboxylic acid (5)

To a solution of methyl 6-(1-(2,6-bis(benzyloxy)pyridin-3-yl)-3-methyl-2-oxo-2,3-dihydro-1H-benzo[d]imidazol-5-yl)-1-methyl-1H-indole-3-carboxylate (4, 700 mg, 1.12 mmol) in methanol (9 mL), THF (9 mL) and water (3 mL) was added NaOH (448.19 mg, 11.21 mmol, 210.42 uL). The mixture was stirred at 70° C. for 12 h. The reaction mixture was concentrated to remove THF and MeOH, then the residue was acidified to pH=3 with 1N HCl, yellow precipitate was formed, filtered and the filter cake was collected to give 6-(1-(2,6-bis(benzyloxy)pyridin-3-yl)-3-methyl-2-oxo-2,3-dihydro-1H-benzo[d]imidazol-5-yl)-1-methyl-1H-indole-3-carboxylic acid (5, 600 mg, 903.94 μmol, 81% yield) as a yellow solid.

LCMS (ESI): m/z 611.4 [M+H]⁺

### Step 4: 6-(1-(2,6-bis(benzyloxy)pyridin-3-yl)-3-methyl-2-oxo-2,3-dihydro-1H-benzo[d]imidazol-5-yl)-1-methyl-1H-indole-3-carboxamide (6)

To a solution of 6-(1-(2,6-bis(benzyloxy)pyridin-3-yl)-3-methyl-2-oxo-2,3-dihydro-1H-benzo[d] imidazol-5-yl)-1-methyl-1H-indole-3-carboxylic acid (5, 600 mg, 982.55 μmol) in DMF (35 mL) was added HOBt·NH₃ (224.24 mg, 1.47 mmol) and 3-(ethyliminomethyleneamino)-N,N-dimethyl-propan-1-amine; hydrochloride (282.53 mg, 1.47 mmol) and DIPEA (253.97 mg, 1.97 mmol, 342.28 uL). The mixture was stirred at 25° C. for 24 h. The reaction mixture was diluted with H₂O (30 mL) and extracted with ethyl acetate (30 mL×2). The combined organic layers were washed with aqueous NaCl (15 mL×2), dried over Na₂SO₄, filtered and concentrated under reduced pressure. The residue was triturated with petroleum ether/ethyl acetate (20 mL, 3/1) to afford 6-(1-(2,6-bis(benzyloxy)pyridin-3-yl)-3-methyl-2-oxo-2,3-dihydro-1H-benzo[d]imidazol-5-yl)-1-methyl-1H-indole-3-carboxamide (6, 400 mg, 590.48 μmol, 60% yield) as a white solid.

LCMS (ESI): m/z 610.3 [M+H]⁺

### 2-(5-(1-(2,6-bis(benzyloxy)pyridin-3-yl)-3-methyl-2-oxo-2,3-dihydro-1H-benzo[d]imidazol-5-yl)-6-fluoro-1H-indazol-1-yl)acetic acid (6)

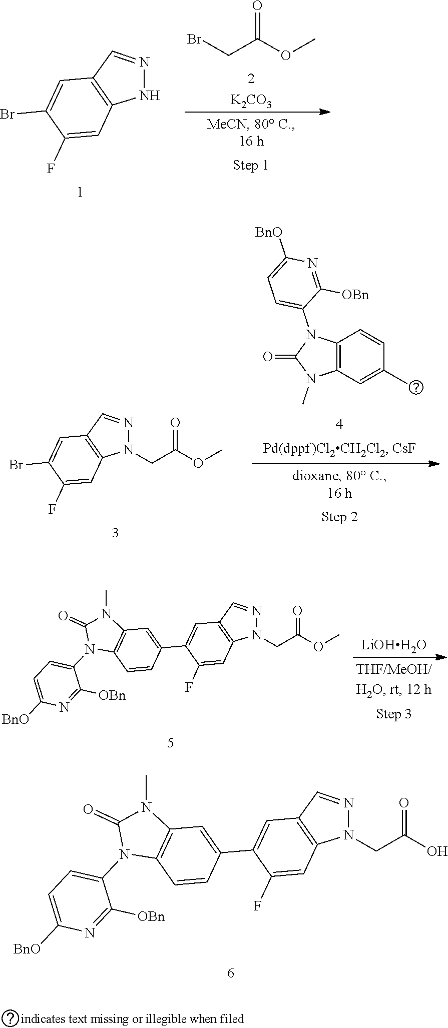

### Step 1: methyl 2-(5-bromo-6-fluoro-1H-indazol-1-yl)acetate (3)

5-bromo-6-fluoro-1H-indazole (1, 1 g, 4.65 mmol) and methyl 2-bromoacetate (2, 853.72 mg, 5.58 mmol, 514.29 μL) are mixed in ACN (20 mL). K₂CO₃ (1.93 g, 13.95 mmol) was added to the reaction mixture, and the mixture was stirred at 80° C. for 12 h. The reaction mixture was cooled to room temperature. EtOAc (40 mL) and water (40 mL) were added and layers were separated. The aqueous phase was extracted with EtOAc (30 mL×2). Combined extracts were washed with brine (60 mL), dried over Na₂SO₄, filtered, and concentrated under vacuum. The residue was purified on automated flash chromatography system (ethyl acetate/petroleum ether from 0:1 to 1:1) to give methyl 2-(5-bromo-6-fluoro-1H-indazol-1-yl)acetate (3, 900 mg, 2.98 mmol, 64% yield) as white solid.

¹H NMR (400 MHz, CDCl3) δ=8.03-7.93 (m, 2H), 7.12-7.09 (m, 1H), 5.12 (s, 2H), 3.78 (s, 3H).

### Step 2: methyl 2-(5-(1-(2,6-bis(benzyloxy)pyridin-3-yl)-3-methyl-2-oxo-2,3-dihydro-1H-benzo[d]imidazol-5-yl)-6-fluoro-1H-indazol-1-yl)acetate (5)

To a suspension of methyl 2-(5-bromo-6-fluoro-1H-indazol-1-yl)acetate (3, 0.5 g, 1.74 mmol) and Pd(dppf)Cl₂·CH₂Cl₂ (142.23 mg, 174.16 μmol) in dioxane (5 mL) at 25° C. was added 1-(2,6-bis(benzyloxy)pyridin-3-yl)-3-methyl-5-(4,4,5,5-tetramethyl-1,3,2-dioxaborolan-2-yl)-1H-benzo[d]imidazol-2 (3H)-one (1.18 g, 2.09 mmol), CsF (793.69 mg, 5.22 mmol) under nitrogen. The reaction mixture was warmed up to 80° C. for 16 h. The reaction mixture was cooled to room temperature. EtOAc (20 mL) and water (20 mL) were added and layers were separated. The aqueous phase was extracted with EtOAc (20 mL×2). Combined extracts were washed with brine (50 mL×3), dried over Na₂SO₄, filtered, and concentrated under vacuum. The residue was purified on automated flash chromatography system (ethyl acetate/petroleum ether from 0:1 to 1:1) to give methyl 2-(5-(1-(2,6-bis(benzyloxy)pyridin-3-yl)-3-methyl-2-oxo-2,3-dihydro-1H-benzo[d]imidazol-5-yl)-6-fluoro-1H-indazol-1-yl)acetate (5, 0.9 g, 1.26 mmol, 72% yield) as brown oil.

LCMS (ESI): m/z 643.8 [M+H]⁺

### Step 3: 2-(5-(1-(2,6-bis(benzyloxy)pyridin-3-yl)-3-methyl-2-oxo-2,3-dihydro-1H-benzo[d]imidazol-5-yl)-6-fluoro-1H-indazol-1-yl)acetic acid (6)

LiOH·H₂O (159.07 mg, 3.88 mmol) was added to a solution of methyl 2-[5-[1-(2,6-dibenzyloxy-3-pyridyl)-3-methyl-2-oxo-benzimidazol-5-yl]-6-fluoro-indazol-1-yl]acetate (5, 0.5 g, 776.81 μmol) in Water (4 mL) and Methanol (4 mL) THF (4 mL). The resulting mixture was stirred at 20° C. for 12 hrs. The reaction mixture was acidized with 1 M HCl to pH=5-6. The mixture was extracted with EtOAc (30 mL×3). The combined organic phase were washed with water (50 mL) and saturated brine (50 mL) and then dried over Na₂SO₄, filtered and concentrated in vacuum to afford 2-[5-[1-(2,6-dibenzyloxy-3-pyridyl)-3-methyl-2-oxo-benzimidazol-5-yl]-6-fluoro-indazol-1-yl]acetic acid (6, 450 mg, 664.67 μmol, 86% yield) as a yellow solid.

LCMS (ESI): m/z 629.8 [M+H]⁺

### 1-(2,6-bis(benzyloxy)pyridin-3-yl)-3-methyl-5-(2,7-diazaspiro[3.5]nonan-7-yl)-1H-benzo[d]imidazol-2 (3H)-one (4)

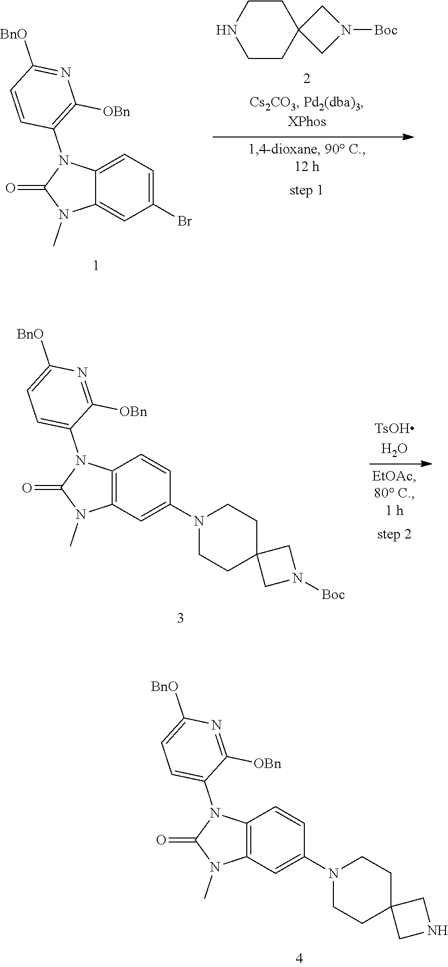 [SMILES: Cn1c(=O)n(-c2ccc(OCc3ccccc3)nc2OCc2ccccc2)c2ccc(Br)cc21 ; CC(C)(C)OC(=O)N1CC2(CCNCC2)C1 ; Cn1c(=O)n(-c2ccc(OCc3ccccc3)nc2OCc2ccccc2)c2ccc(N3CCC4(CC3)CN(C(=O)OC(C)(C)C)C4)cc21 ; Cn1c(=O)n(-c2ccc(OCc3ccccc3)nc2OCc2ccccc2)c2ccc(N3CCC4(CC3)CNC4)cc21]

### Step 1: tert-butyl 7-(1-(2,6-bis(benzyloxy)pyridin-3-yl)-3-methyl-2-oxo-2,3-dihydro-1H-benzo[d]imidazol-5-yl)-2,7-diazaspiro[3.5]nonane-2-carboxylate (3)

To a mixture of 1-(2,6-bis(benzyloxy)pyridin-3-yl)-5-bromo-3-methyl-1H-benzo[d]imidazol-2 (3H)-one (1, 1 g, 1.94 mmol), tert-butyl 2,7-diazaspiro[3.5]nonane-2-carboxylate (2, 482.09 mg, 2.13 mmol), Pd₂(dba)₃ (177.33 mg, 193.65 μmol), XPhos (184.64 mg, 387.31 μmol) and Cs₂CO₃ (1.89 g, 5.81 mmol) in dioxane (10 mL) under N₂. The mixture was stirred at 90° C. for 12 h under N₂. The reaction mixture was concentrated under vacuum. The residue was purified by flash silica gel chromatography (flow: 50 mL/min; gradient: 0-100% ethylacetate in petroleum ether; ISCO®; 40 g SepaFlash®Silica Flash Column; ethylacetate/petroleumether=1/0) to afford tert-butyl 7-(1-(2,6-bis(benzyloxy)pyridin-3-yl)-3-methyl-2-oxo-2,3-dihydro-1H-benzo[d]imidazol-5-yl)-2,7-diazaspiro[3.5]nonane-2-carboxylate (3, 1.1 g, 1.64 mmol, 85% yield) as a yellow solid.

LCMS (ESI): m/z 662.4 [M+H]⁺

¹HNMR (400 MHz, DMSO-d6) δ 7.76 (d, J=8.0 Hz, 1H), 7.48-7.22 (m, 10H), 6.86 (d, J=2.0 Hz, 1H), 6.64-6.48 (m, 3H), 5.43-5.29 (m, 4H), 3.59 (br s, 4H), 3.34 (s, 3H), 3.03 (br s, 4H), 1.79 (m, 4H), 1.40-1.37 (m, 9H).

### Step 2: 1-(2,6-bis(benzyloxy)pyridin-3-yl)-3-methyl-5-(2,7-diazaspiro[3.5]nonan-7-yl)-1H-benzo[d]imidazol-2 (3H)-one (4)

To a mixture of tert-butyl 7-[1-(2,6-dibenzyloxy-3-pyridyl)-3-methyl-2-oxo-benzimidazol-5-yl]-2,7-diazaspiro[3.5]nonane-2-carboxylate (3, 700 mg, 1.06 mmol) in EtOAc (7 mL) was added P-TOLUENESULFONIC ACID MONOHYDRATE (402.40 mg, 2.12 mmol, 324.52 uL). The mixture was stirred at 80° C. for 1 h. The reaction mixture was concentrated to get a residue. The mixture was purified by reversed phase HPLC (FA) and then lyophilization. The 1-(2,6-bis(benzyloxy)pyridin-3-yl)-3-methyl-5-(2,7-diazaspiro[3.5]nonan-7-yl)-1H-benzo[d]imidazol-2 (3H)-one (4, 520 mg, 812.91 μmol, 77% yield, formic acid salt) as yellow solid.

LCMS (ESI): m/z 562.2 [M+H]⁺

### 2-[4-[1-(2,6-dioxo-3-piperidyl)-3-methyl-indazol-5-yl]phenyl]acetic acid (3)

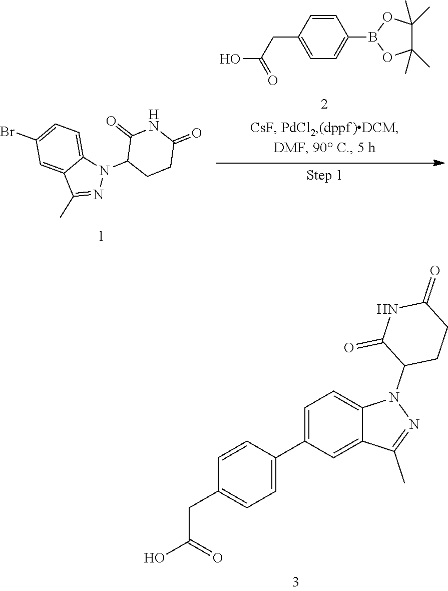 [SMILES: CC1(C)OB(c2ccc(CC(=O)O)cc2)OC1(C)C ; Cc1nn(C2CCC(=O)NC2=O)c2ccc(Br)cc12 ; Cc1nn(C2CCC(=O)NC2=O)c2ccc(-c3ccc(CC(=O)O)cc3)cc12]

### Step 1: 2-[4-[1-(2,6-dioxo-3-piperidyl)-3-methyl-indazol-5-yl]phenyl]acetic acid (3)

To a 10 mL pressure tube containing a well-stirred solution of 3-(5-bromo-3-methyl-indazol-1-yl)piperidine-2,6-dione (1, 200 mg, 620.82 μmol) and 2-[4-(4,4,5,5-tetramethyl-1,3,2-dioxaborolan-2-yl)phenyl]acetic acid (2, 244.08 mg, 931.22 μmol) in anhydrous DMF (3 mL) was added cesium fluoride (235.76 mg, 1.55 mmol) at room temperature. The reaction mixture was degassed by bubbling nitrogen gas for 15 min. Then pd(dppf)c12·DCM (152.09 mg, 186.24 μmol) was added and the reaction mixture was stirred at 90° C. After 5 h, the reaction mixture was filtered and concentrated under reduced pressure. The crude compound was purified by reverse phase column chromatography [Silicycle C18 column; Mobile phase A: 0.1% formic acid in water; Mobile phase B: MeCN] to get 2-[4-[1-(2,6-dioxo-3-piperidyl)-3-methyl-indazol-5-yl]phenyl]acetic acid (3, 90 mg, 29% yield) as white solid.

LCMS (ES+): m/z 378.2 [M+H]⁺

### 2-(4-((1-(2,6-bis(benzyloxy)pyridin-3-yl)-3-methyl-2-oxo-2,3-dihydro-1H-benzo[d]imidazol-5-yl)(methyl)amino)-3-methylphenyl)acetic acid (5)

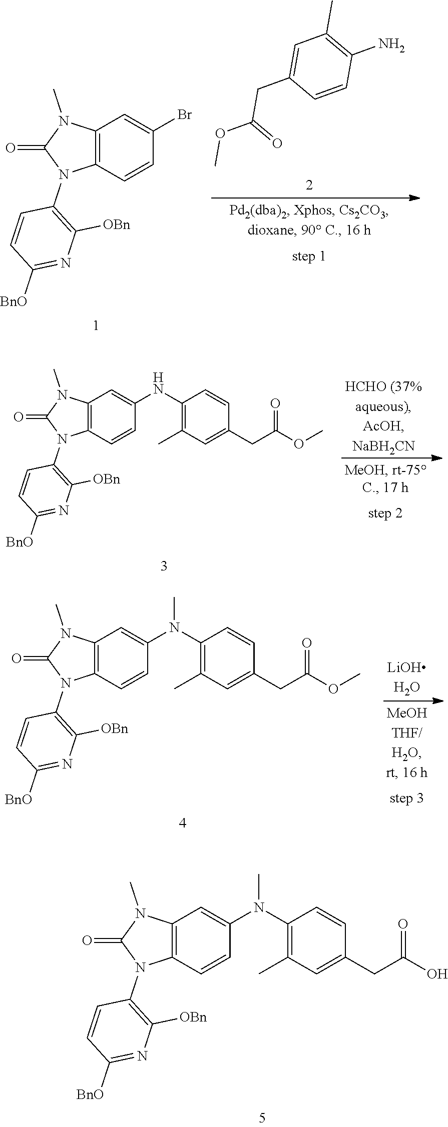 [SMILES: Cn1c(=O)n(-c2ccc(OCc3ccccc3)nc2OCc2ccccc2)c2ccc(Br)cc21 ; COC(=O)Cc1ccc(N)c(C)c1 ; COC(=O)Cc1ccc(Nc2ccc3c(c2)n(C)c(=O)n3-c2ccc(OCc3ccccc3)nc2OCc2ccccc2)c(C)c1 ; "HCHO (37% aqueous),AcOH,NaBH2CN" ; COC(=O)Cc1ccc(N(C)c2ccc3c(c2)n(C)c(=O)n3-c2ccc(OCc3ccccc3)nc2OCc2ccccc2)c(C)c1 ; Cc1cc(CC(=O)O)ccc1N(C)c1ccc2c(c1)n(C)c(=O)n2-c1ccc(OCc2ccccc2)nc1OCc1ccccc1]

### Step 1: methyl 2-(4-((1-(2,6-bis(benzyloxy)pyridin-3-yl)-3-methyl-2-oxo-2,3-dihydro-1H-benzo[d]imidazol-5-yl)amino)-3-methylphenyl)acetate (3)

To a solution of methyl 2-(4-amino-3-methyl-phenyl)acetate (2, 800 mg, 4.46 mmol) and 1-(2,6-bis(benzyloxy)pyridin-3-yl)-5-bromo-3-methyl-1H-benzo[d]imidazol-2 (3H)-one (1, 2.00 g, 3.88 mmol) in dioxane (8 mL) was added dicesium; carbonate (2.53 g, 7.76 mmol) and dicyclohexyl-[2-(2,4,6-triisopropylphenyl)phenyl]phosphane (92.52 mg, 194.08 μmol) and (1E,4E)-1,5-diphenylpenta-1,4-dien-3-one; palladium (177.72 mg, 194.08 μmol). The mixture was stirred at 90° C. for 16 hrs under N₂ atmosphere. The reaction mixture was concentrated under reduced pressure. The residue was purified by column chromatography (SiO₂, petroleum ether/ethyl acetate=10/1 to 1/1) to give methyl 2-(4-((1-(2,6-bis(benzyloxy)pyridin-3-yl)-3-methyl-2-oxo-2,3-dihydro-1H-benzo[d]imidazol-5-yl)amino)-3-methylphenyl)acetate (3, 2 g, 2.99 mmol, 77% yield) as a yellow solid.

LCMS (ESI): m/z 615.2 [M+H]⁺

### Step 2: methyl 2-(4-((1-(2,6-bis(benzyloxy)pyridin-3-yl)-3-methyl-2-oxo-2,3-dihydro-1H-benzo[d]imidazol-5-yl)(methyl)amino)-3-methylphenyl)acetate (4)

To a solution of methyl 2-[4-[[1-(2,6-dibenzyloxy-3-pyridyl)-3-methyl-2-oxo-benzimidazol-5-yl]amino]-3-methyl-phenyl]acetate (3, 0.8 g, 1.30 mmol) in MeOH (16 mL) were added formaldehyde (2.11 g, 26.03 mmol, 1.96 mL) and acetic acid (251.65 mg, 4.19 mmol, 239.67 uL). The mixture was stirred at 30° C. for 1 h. Then sodium; cyanoboranuide (1.64 g, 26.03 mmol) was added and the mixture was stirred at 75° C. for 16 h. The mixture was concentrated under reduced pressure to give a residue. The residue was diluted with ethyl acetate (50 mL) and washed with brine (30 mL×2), dried over Na₂SO₄, filtered and concentrated under reduced pressure to give a residue. The residue was purified by reverse phase (flow: 60 mL/min; gradient: from 5-80% MeCN in water (0.1% TFA) over 45 min; column: 80 g Flash Column, Welch Ultimate XB_C18 20-40 μm; 120 Å). methyl 2-[4-[[1-(2,6-dibenzyloxy-3-pyridyl)-3-methyl-2-oxo-benzimidazol-5-yl]-methyl-amino]-3-methyl-phenyl]acetate (4, 800 mg, 1.11 mmol, 85% yield) as brown oil.

LCMS (ESI): m/z 629.2 [M+H]⁺

### Step 3: 2-(4-((1-(2,6-bis(benzyloxy)pyridin-3-yl)-3-methyl-2-oxo-2,3-dihydro-1H-benzo[d]imidazol-5-yl)(methyl)amino)-3-methylphenyl)acetic acid (5)

To a solution of methyl 2-[4-[[1-(2,6-dibenzyloxy-3-pyridyl)-3-methyl-2-oxo-benzimidazol-5-yl]-methyl-amino]-3-methyl-phenyl]acetate (4, 0.6 g, 954.33 μmol) in THF (2 mL), Methanol (2 mL) and Water (2 mL) was added Lithium hydroxide, monohydrate (200.22 mg, 4.77 mmol, 132.59 uL). The mixture was stirred at 30° C. for 1 h. The mixture was adjusted pH to 4 with 1N HCl aqueous and then extracted with ethyl acetate (20 mL×2). The combined organic layers were washed with brine (30 mL×2), dried over Na₂SO₄, filtered and concentrated under reduced pressure to give 2-[4-[[1-(2,6-dibenzyloxy-3-pyridyl)-3-methyl-2-oxo-benzimidazol-5-yl]-methyl-amino]-3-methyl-phenyl]acetic acid (5, 580 mg, 868.08 μmol, 91% yield) as an off-white solid.

LCMS (ESI): m/z 615.2 [M+H]+

### 2-(1-(1-(2,6-bis(benzyloxy)pyridin-3-yl)-3-methyl-2-oxo-2,3-dihydro-1H-benzo[d]imidazol-5-yl)-1H-pyrazol-3-yl)acetic acid (4)

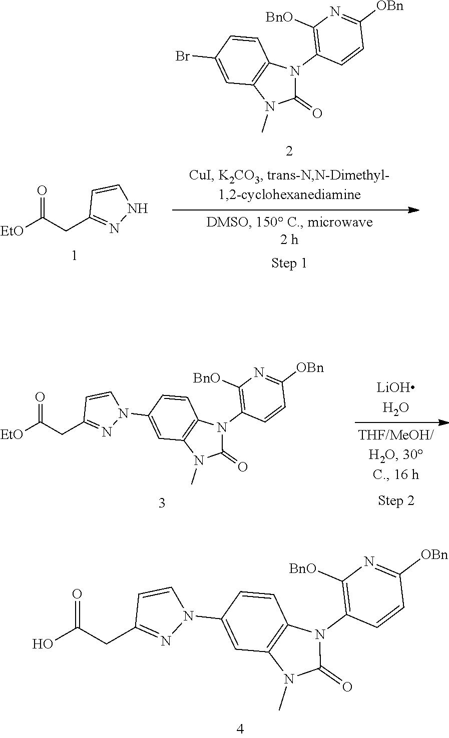 [SMILES: Cn1c(=O)n(-c2ccc(OCc3ccccc3)nc2OCc2ccccc2)c2ccc(Br)cc21 ; CCOC(=O)Cc1cc[nH]n1 ; CCOC(=O)Cc1ccn(-c2ccc3c(c2)n(C)c(=O)n3-c2ccc(OCc3ccccc3)nc2OCc2ccccc2)n1 ; Cn1c(=O)n(-c2ccc(OCc3ccccc3)nc2OCc2ccccc2)c2ccc(-n3ccc(CC(=O)O)n3)cc21]

### Step 1: ethyl 2-(1-(1-(2,6-bis(benzyloxy)pyridin-3-yl)-3-methyl-2-oxo-2,3-dihydro-1H-benzo[d]imidazol-5-yl)-1H-pyrazol-3-yl)acetate (3)

The mixture of ethyl 2-(1H-pyrazol-3-yl)acetate (1, 179.13 mg, 1.16 mmol), 1-(2,6-bis(benzyloxy)pyridin-3-yl)-5-bromo-3-methyl-1H-benzo[d]imidazol-2 (3H)-one (2, 500 mg, 968.27 μmol), iodocopper (184.41 mg, 968.27 μmol, 32.81 uL), dipotassium; carbonate (401.46 mg, 2.90 mmol, 175.31 μL) and (1S,2S)—N1,N2-dimethylcyclohexane-1,2-diamine (137.73 mg, 968.27 μmol, 152.69 μL) in DMSO (5 mL) was microwaved to 150° C. for 2 hr under N₂ atmosphere. The reaction mixture was poured into water (20 mL) and extracted by EA (20 mL×3). The combined organic layer was washed by brine (10 mL), dried by anhydrous Na₂SO₄, filtered and concentrated to afford residue. The residue was purified by flash silica gel chromatography (20 g Silica Flash Column, Eluent of 20˜50% Ethylacetate/Petroleum ether gradient @ 35 mL/min). The eluent was concentrated to afford ethyl 2-(1-(1-(2,6-bis(benzyloxy)pyridin-3-yl)-3-methyl-2-oxo-2,3-dihydro-1H-benzo[d]imidazol-5-yl)-1H-pyrazol-3-yl)acetate (3, 300 mg, 432.47 μmol, 45% yield) as brown oil.

LCMS (ESI): m/z 589.9 [M+H]⁺

¹H NMR (400 MHz, DMSO-d₆) δ=8.39 (d, J=2.4 Hz, 1H), 7.84 (d, J=8.4 Hz, 1H), 7.66 (d, J=2.0 Hz, 1H), 7.47-7.34 (m, 6H), 7.30-7.22 (m, 5H), 6.77 (d, J=8.4 Hz, 1H), 6.63 (d, J=8.4 Hz, 1H), 6.44 (d, J=2.4 Hz, 1H), 5.47-5.30 (m, 4H), 4.05-4.00 (m, 2H), 3.74 (s, 2H), 3.44 (s, 3H), 1.19-1.15 (m, 3H).

### Step 2: 2-(1-(1-(2,6-bis(benzyloxy)pyridin-3-yl)-3-methyl-2-oxo-2,3-dihydro-1H-benzo[d]imidazol-5-yl)-1H-pyrazol-3-yl)acetic acid (4)

To a solution of ethyl 2-[1-[1-(2,6-dibenzyloxy-3-pyridyl)-3-methyl-2-oxo-benzimidazol-5-yl]pyrazol-3-yl]acetate (3, 300 mg, 508.79 μmol) and THF (1.5 mL) in MeOH (3 mL) was added a solution of lithium; hydroxide; hydrate (106.75 mg, 2.54 mmol) in water (1.5 mL). The reaction mixture was stirred at 30° C. for 16 h. The reaction mixture was poured into water (5 mL) and added 1M HCl aqueous to adjusted pH=7. The mixture was extracted by EA (5 mL×3). The combined organic layer was washed with brine (5 mL), dried over anhydrous sodium sulfate, filtered and concentrated to give 2-[1-[1-(2,6-dibenzyloxy-3-pyridyl)-3-methyl-2-oxo-benzimidazol-5-yl]pyrazol-3-yl]acetic acid (4, 280 mg, 423.80 μmol, 83% yield) as white solid.

LCMS (ESI): m/z 561.9 [M+H]⁺

### 5-(4-(aminomethyl)piperidin-1-yl)-1-(2,6-bis(benzyloxy)pyridin-3-yl)-3-methyl-1H-benzo[d]imidazol-2 (3H)-one (4)

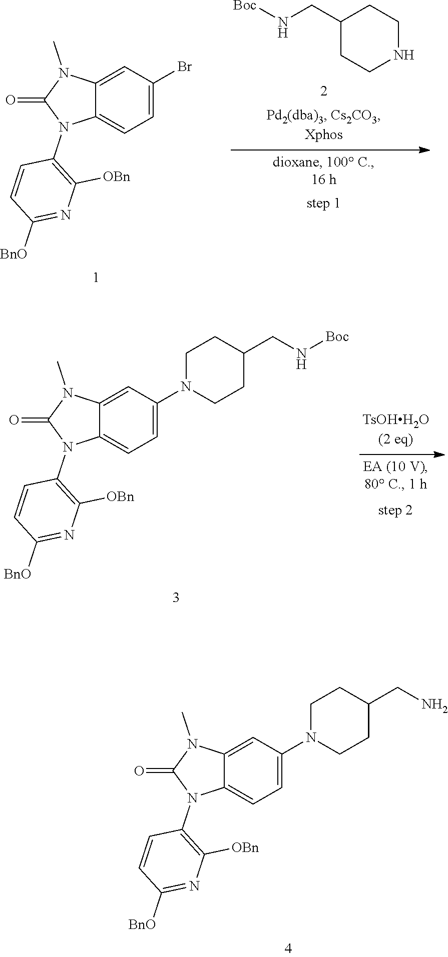 [SMILES: Cn1c(=O)n(-c2ccc(OCc3ccccc3)nc2OCc2ccccc2)c2ccc(Br)cc21 ; CC(C)(C)OC(=O)NCC1CCNCC1 ; Cn1c(=O)n(-c2ccc(OCc3ccccc3)nc2OCc2ccccc2)c2ccc(N3CCC(CNC(=O)OC(C)(C)C)CC3)cc21 ; Cn1c(=O)n(-c2ccc(OCc3ccccc3)nc2OCc2ccccc2)c2ccc(N3CCC(CN)CC3)cc21]

### Step 1: tert-butyl ((1-(1-(2,6-bis(benzyloxy)pyridin-3-yl)-3-methyl-2-oxo-2,3-dihydro-1H-benzo[d]imidazol-5-yl)piperidin-4-yl)methyl)carbamate (3)

A mixture of 1-(2,6-bis(benzyloxy)pyridin-3-yl)-5-bromo-3-methyl-1H-benzo[d]imidazol-2 (3H)-one (1, 1 g, 1.94 mmol), tert-butyl N-(4-piperidylmethyl)carbamate (539.51 mg, 2.52 mmol), dicesium; carbonate (1.89 g, 5.81 mmol) and dicyclohexyl-[2-(2,4,6-Triisopropyl phenyl)phenyl]phosphane (2, 184.64 mg, 387.31 μmol) in dioxane (10 mL) was added (1E,4E)-1,5-diphenylpenta-1,4-dien-3-one; palladium (177.33 mg, 193.65 μmol) and degassed and purged with N₂ 3 times, and then the mixture was stirred at 100° C. for 16 hr under N₂ atmosphere. The mixture was filtered and the filtrate was concentrated in vacuum. The residue was purified by flash silica gel chromatography (SiO₂, petroleum ether/ethylacetate=1/0 to 0/1) to afford tert-butyl ((1-(1-(2,6-bis(benzyloxy)pyridin-3-yl)-3-methyl-2-oxo-2,3-dihydro-1H-benzo[d]imidazol-5-yl)piperidin-4-yl)methyl)carbamate (3, 700 mg, 831.40 μmol, 43% yield) as a yellow oil.

LCMS (ESI): m/z 650.6 [M+H]⁺

### Step 2: 5-(4-(aminomethyl)piperidin-1-yl)-1-(2,6-bis(benzyloxy)pyridin-3-yl)-3-methyl-1H-benzo[d]imidazol-2 (3H)-one (4)

To a solution of tert-butyl ((1-(1-(2,6-bis(benzyloxy)pyridin-3-yl)-3-methyl-2-oxo-2,3-dihydro-1H benzo[d]imidazol-5-yl)piperidin-4-yl)methyl)carbamate (3, 580 mg, 892.61 μmol) in ethyl acetate (6 mL) was added 4-methylbenzenesulfonic acid (307.42 mg, 1.79 mmol). The mixture was stirred at 80° C. for 2 h. the reaction mixture was purified by reversed phase (0.1% FA) to obtain 5-(4-(aminomethyl) piperidin-1-yl)-1-(2,6-bis(benzyloxy)pyridin-3-yl)-3-methyl-1H-benzo[d]imidazol-2 (3H)-one (4, 400 mg, 526.42 μmol, 59% yield, TsOH salt) as a red solid.

LCMS (ESI): m/z 550.2 [M+H]⁺

### (S)-1-(2,6-bis(benzyloxy)pyridin-3-yl)-3-methyl-5-(3-methylpiperazin-1-yl)-1H-benzo[d]imidazol-2 (3H)-one (4)

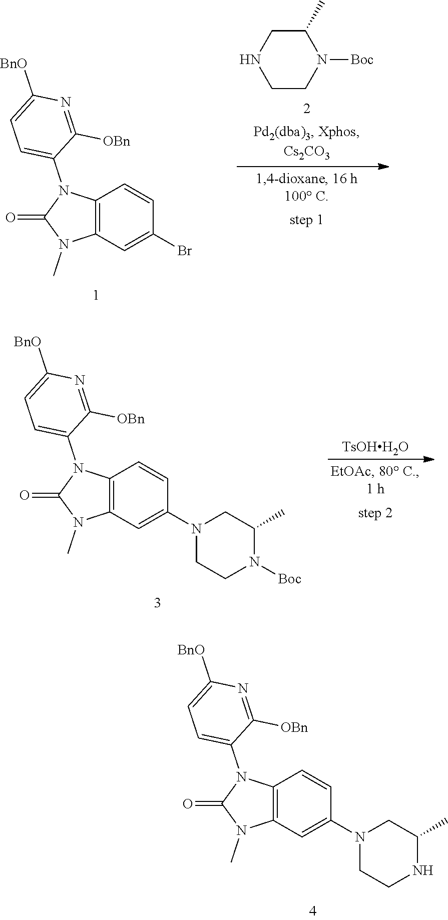 [SMILES: Cn1c(=O)n(-c2ccc(OCc3ccccc3)nc2OCc2ccccc2)c2ccc(Br)cc21 ; C[C@H]1CNCCN1C(=O)OC(C)(C)C ; C[C@H]1CN(c2ccc3c(c2)n(C)c(=O)n3-c2ccc(OCc3ccccc3)nc2OCc2ccccc2)CCN1C(=O)OC(C)(C)C ; "TsOH·H2O" ; C[C@H]1CN(c2ccc3c(c2)n(C)c(=O)n3-c2ccc(OCc3ccccc3)nc2OCc2ccccc2)CCN1]

### Step 1: (S)-tert-butyl 4-(1-(2,6-bis(benzyloxy)pyridin-3-yl)-3-methyl-2-oxo-2,3-dihydro-1H-benzo[d]imidazol-5-yl)-2-methylpiperazine-1-carboxylate (3)

To a mixture of 1-(2,6-bis(benzyloxy)pyridin-3-yl)-5-bromo-3-methyl-1H-benzo[d]imidazol-2 (3H)-one (1, 1 g, 1.94 mmol), (S)-tert-butyl 2-methylpiperazine-1-carboxylate (2, 426.63 mg, 2.13 mmol), Pd₂(dba)₃ (1.77 g, 1.94 mmol), XPhos (923.18 mg, 1.94 mmol) and Cs₂CO₃ (630.96 mg, 1.94 mmol) in dioxane (10 mL) under N₂. The mixture was stirred at 90° C. for 12 hr under N₂. The reaction mixture was concentrated under vacuum. The residue was purified by flash silica gel chromatography (flow: 50 mL/min; gradient: 0-100% ethylacetate in petroleum ether; ISCO®; 40 g SepaFlash®Silica Flash Column; ethylacetate/petroleumether=1/0) to afford (S)-tert-butyl 4-(1-(2,6-bis(benzyloxy)pyridin-3-yl)-3-methyl-2-oxo-2,3-dihydro-1H-benzo[d]imidazol-5-yl)-2-methylpiperazine-1-carboxylate (3, 0.94 g, 1.47 mmol, 76% yield) as a yellow solid.

LCMS (ESI): m/z 636.4 [M+H]⁺

¹HNMR (400 MHz, DMSO-d₆) δ=7.76 (d, J=8.4 Hz, 1H), 7.27 (m, 10H), 6.86 (d, J=2.0 Hz, 1H), 6.67-6.51 (m, 3H), 5.44-5.30 (m, 4H), 4.22 (br s, 1H), 3.81 (br d, J=13.2 Hz, 1H), 3.48 (br d, J=11.6 Hz, 1H), 3.36 (s, 3H), 3.17 (br d, J=2.4 Hz, 1H), 2.76 (m, 1H), 1.99 (s, 2H), 1.46-1.39 (m, 9H), 1.24 (d, J=6.8 Hz, 3H).

### Step 2: (S)-1-(2,6-bis(benzyloxy)pyridin-3-yl)-3-methyl-5-(3-methylpiperazin-1-yl)-1H-benzo[d]imidazol-2 (3H)-one (4)

To a mixture of tert-butyl (2S)-4-[1-(2,6-dibenzyloxy-3-pyridyl)-3-methyl-2-oxo-benzimidazol-5-yl]-2-methyl-piperazine-1-carboxylate (3, 200 mg, 314.59 μmol) in EtOAc (2 mL) was added P-TOLUENESULFONIC ACID MONOHYDRATE (119.68 mg, 629.18 μmol, 96.52 uL). The mixture was stirred at 80° C. for 1 h. The reaction mixture was quenched by addition of saturated NaHCO₃ (5 mL) aqueous at 0° C., and then diluted with water (5 mL) and extracted with ethyl acetate (10 mL×3). The combined organic layers were washed with brine (10 mL), dried by anhydrous Na₂SO₄, filtered and concentrated in vacuum to afford (S)-1-(2,6-bis(benzyloxy)pyridin-3-yl)-3-methyl-5-(3-methylpiperazin-1-yl)-1H-benzo[d]imidazol-2 (3H)-one (4, 160 mg, 268.84 μmol, 85% yield) as yellow solid.

LCMS (ESI): m/z 536.2 [M+H]⁺

### (R)-1-(2,6-bis(benzyloxy)pyridin-3-yl)-3-methyl-5-(3-methylpiperazin-1-yl)-1H-benzo[d]imidazol-2 (3H)-one (4)

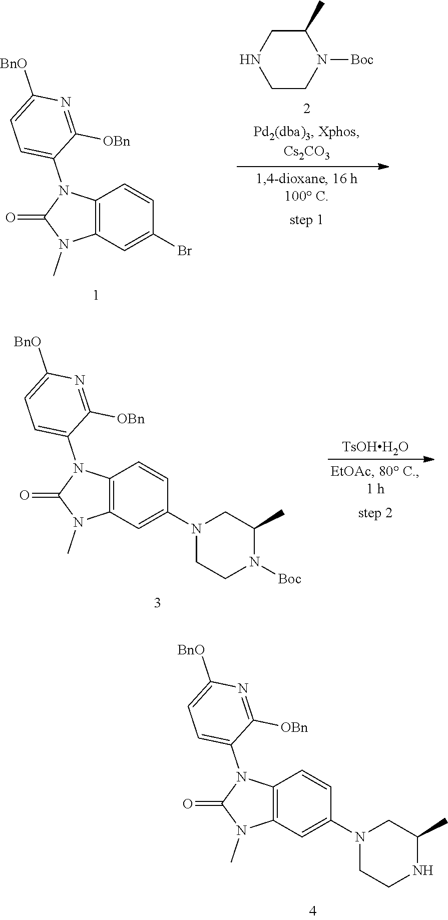 [SMILES: Cn1c(=O)n(-c2ccc(OCc3ccccc3)nc2OCc2ccccc2)c2ccc(Br)cc21 ; C[C@@H]1CNCCN1C(=O)OC(C)(C)C ; C[C@@H]1CN(c2ccc3c(c2)n(C)c(=O)n3-c2ccc(OCc3ccccc3)nc2OCc2ccccc2)CCN1C(=O)OC(C)(C)C ; "TsOH·H2O" ; C[C@@H]1CN(c2ccc3c(c2)n(C)c(=O)n3-c2ccc(OCc3ccccc3)nc2OCc2ccccc2)CCN1]

### Step 1: (R)-tert-butyl 4-(1-(2,6-bis(benzyloxy)pyridin-3-yl)-3-methyl-2-oxo-2,3-dihydro-1H-benzo[d]imidazol-5-yl)-2-methylpiperazine-1-carboxylate (3)

To a mixture of 1-(2,6-bis(benzyloxy)pyridin-3-yl)-5-bromo-3-methyl-1H-benzo[d]imidazol-2 (3H)-one (1, 1 g, 1.94 mmol), (R)-tert-butyl 2-methylpiperazine-1-carboxylate (2, 426.63 mg, 2.13 mmol), Pd₂(dba)₃ (1.77 g, 1.94 mmol), XPhos (923.18 mg, 1.94 mmol) and Cs₂CO₃ (630.96 mg, 1.94 mmol) in dioxane (10 mL) under N₂. The mixture was stirred at 90° C. for 12 hr under N₂. The reaction mixture was concentrated under vacuum. The residue was purified by flash silica gel chromatography (flow: 50 mL/min; gradient: 0-100% ethylacetate in petroleum ether; ISCO®; 40 g SepaFlash®Silica Flash Column; ethylacetate/petroleumether=1/0) to afford (R)-tert-butyl 4-(1-(2,6-bis(benzyloxy)pyridin-3-yl)-3-methyl-2-oxo-2,3-dihydro-1H-benzo[d]imidazol-5-yl)-2-methylpiperazine-1-carboxylate (3, 0.99 g, 1.26 mmol, 65% yield) as a yellow solid.

LCMS (ESI): m/z 636.4 [M+H]⁺

¹HNMR (400 MHz, DMSO-d₆) δ=7.76 (d, J=8.4 Hz, 1H), 7.48-7.23 (m, 10H), 6.86 (d, J=2.0 Hz, 1H), 6.65-6.51 (m, 3H), 5.42-5.27 (m, 4H), 4.21 (br s, 1H), 3.81 (br d, J=13.2 Hz, 1H), 3.48 (br d, J=11.2 Hz, 1H), 3.36 (s, 3H), 3.17 (br d, J=2.4 Hz, 1H), 2.76 (m, 1H), 1.99 (s, 2H), 1.46-1.40 (m, 9H), 1.24 (d, J=6.8 Hz, 3H).

### Step 2: (R)-1-(2,6-bis(benzyloxy)pyridin-3-yl)-3-methyl-5-(3-methylpiperazin-1-yl)-1H-benzo[d]imidazol-2 (3H)-one (4)

To a mixture of tert-butyl (2R)-4-[1-(2,6-dibenzyloxy-3-pyridyl)-3-methyl-2-oxo-benzimidazol-5-yl]-2-methyl-piperazine-1-carboxylate (3, 200 mg, 314.59 μmol) in EtOAc (2 mL) was added P-TOLUENESULFONIC ACID MONOHYDRATE (119.68 mg, 629.18 μmol, 96.52 uL). The mixture was stirred at 80° C. for 1 h. The reaction mixture was quenched by addition of saturated NaHCO₃ (5 mL) aqueous at 0° C., and then diluted with water (5 mL) and extracted with ethyl acetate (10 mL×3). The combined organic layers were washed with brine (10 mL), dried by anhydrous Na₂SO₄, filtered and concentrated in vacuum to afford (R)-1-(2,6-bis(benzyloxy)pyridin-3-yl)-3-methyl-5-(3-methylpiperazin-1-yl)-1H-benzo[d]imidazol-2 (3H)-one (4, 160 mg, 268.84 μmol, 85% yield) as yellow solid.

LCMS (ESI): m/z 536.2 [M+H]⁺

### 2-(2-(1-(2,6-bis(benzyloxy)pyridin-3-yl)-3-methyl-2-oxo-2,3-dihydro-1H-benzo[d]imidazol-5-yl)-2,7-diazaspiro[3.5]nonan-7-yl)acetic acid (6)

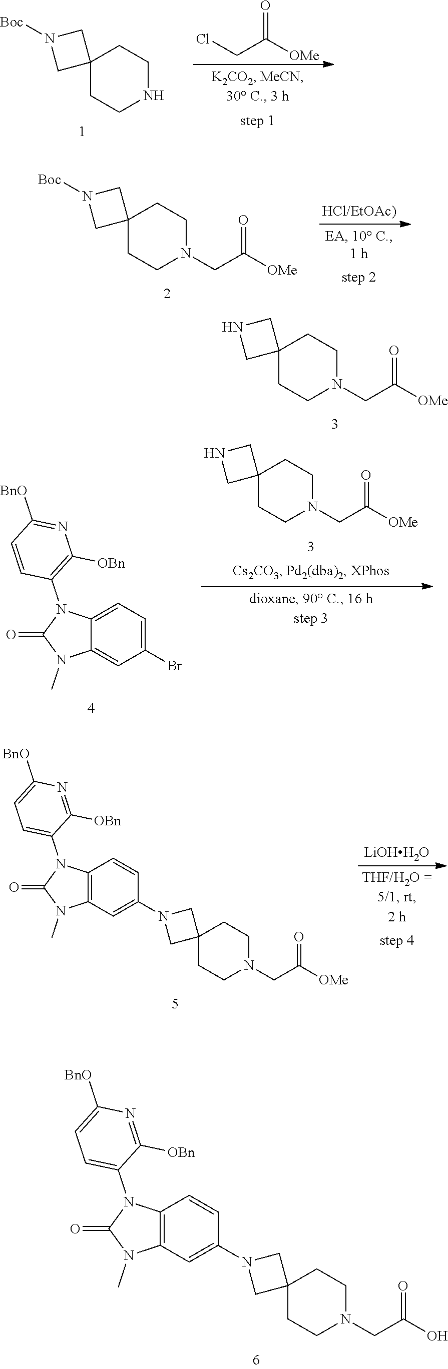

### Step 1: tert-butyl 7-(2-methoxy-2-oxoethyl)-2,7-diazaspiro[3.5]nonane-2-carboxylate (2)

To a solution of tert-butyl 2,7-diazaspiro[3.5]nonane-2-carboxylate (1, 1 g, 4.42 mmol) in MeCN (10 mL) was added K₂CO₃ (1.83 g, 13.26 mmol, 800.02 uL) and methyl 2-chloroacetate (719.29 mg, 6.63 mmol, 580.07 uL). The mixture was stirred at 30° C. for 3 h. The reaction mixture was filtered and concentrated under reduced pressure to give tert-butyl 7-(2-methoxy-2-oxoethyl)-2,7-diazaspiro[3.5]nonane-2-carboxylate (2, 1.4 g, 4.22 mmol, 96% yield) as colorless oil.

¹HNMR (400 MHz, DMSO-d₆) δ 3.59 (s, 3H), 3.50 (br s, 4H), 3.17 (s, 2H), 2.38 (br s, 4H), 1.64 (m, 4H), 1.37 (s, 9H).

### Step 2: methyl 2-(2,7-diazaspiro[3.5]nonan-7-yl)acetate (3)

To a solution of tert-butyl 7-(2-methoxy-2-oxo-ethyl)-2,7-diazaspiro[3.5]nonane-2-carboxylate (2, 1.4 g, 4.69 mmol) in EtOAc (5 mL) was added HCl/EtOAc (4 M, 10 mL). The mixture was stirred at 30° C. for 0.5 h. The reaction mixture was concentrated under reduced pressure to give methyl 2-(2,7-diazaspiro[3.5]nonan-7-yl)acetate (3, 1.5 g, 4.47 mmol, 95% yield, HCl salt) as white solid.

¹HNMR (400 MHz, DMSO-d₆) δ 10.86 (br s, 1H), 9.63 (br s, 2H), 4.17 (s, 2H), 3.82-3.74 (m, 5H), 3.70 (br s, 2H), 3.45 (br d, J=11.6 Hz, 2H), 3.12 (br s, 2H), 2.29-2.18 (m, 2H), 2.03 (br d, J=12.4 Hz, 2H).

### Step 3: methyl 2-(2-(1-(2,6-bis(benzyloxy)pyridin-3-yl)-3-methyl-2-oxo-2,3-dihydro-1H-benzo[d]imidazol-5-yl)-2,7-diazaspiro[3.5]nonan-7-yl)acetate (5)

To a mixture of 1-(2,6-bis(benzyloxy)pyridin-3-yl)-5-bromo-3-methyl-1H-benzo[d]imidazol-2 (3H)-one (4, 1 g, 1.94 mmol), methyl 2-(2,7-diazaspiro[3.5]nonan-7-yl)acetate (3, 682 mg, 2.91 mmol, 021), Pd₂(dba)₃ (177.33 mg, 193.65 μmol), XPhos (184.64 mg, 387.31 μmol) and Cs₂CO₃ (1.89 g, 5.81 mmol) in dioxane (10 mL) under N₂. The mixture was stirred at 90° C. for 12 hr under N₂. The reaction mixture was concentrated under vacuum. The residue was purified by flash silica gel chromatography (flow: 50 mL/min; gradient: 0-100% ethylacetate in petroleum ether; ISCO®; 40 g SepaFlash®Silica Flash Column; ethylacetate/petroleumether=1/0) to afford methyl 2-(2-(1-(2,6-bis(benzyloxy)pyridin-3-yl)-3-methyl-2-oxo-2,3-dihydro-1H-benzo[d]imidazol-5-yl)-2,7-diazaspiro[3.5]nonan-7-yl)acetate (5, 610 mg, 770.04 μmol, 40% yield) as a yellow solid.

LCMS (ESI): m/z 634.6 [M+H]⁺

### Step 4: 2-(2-(1-(2,6-bis(benzyloxy)pyridin-3-yl)-3-methyl-2-oxo-2,3-dihydro-1H-benzo[d]imidazol-5-yl)-2,7-diazaspiro[3.5]nonan-7-yl)acetic acid (6)

To a solution of methyl 2-(2-(1-(2,6-bis(benzyloxy)pyridin-3-yl)-3-methyl-2-oxo-2,3-dihydro-1H-benzo[d]imidazol-5-yl)-2,7-diazaspiro[3.5]nonan-7-yl)acetate (5, 610 mg, 962.55 μmol) in THF (6 mL) was added a solution of LiOH·H₂O (605.88 mg, 14.44 mmol) in Water (6 mL). The mixture was stirred at 25° C. for 2 h. The reaction mixture was concentrated under vacuum. The residue was purified by reversed phase HPLC (FA) and then lyophilisation to afford 2-(2-(1-(2,6-bis(benzyloxy)pyridin-3-yl)-3-methyl-2-oxo-2,3-dihydro-1H-benzo[d]imidazol-5-yl)-2,7-diazaspiro[3.5]nonan-7-yl)acetic acid (6, 220 mg, 355.01 μmol, 37% yield) as a white solid: LCMS (ESI): m/z 620.2 [M+H]⁺

### 3-(4-(1-(2,6-bis(benzyloxy)pyridin-3-yl)-3-methyl-2-oxo-2,3-dihydro-1H-benzo[d]imidazol-5-yl)piperazin-1-yl)propanoic acid (7)

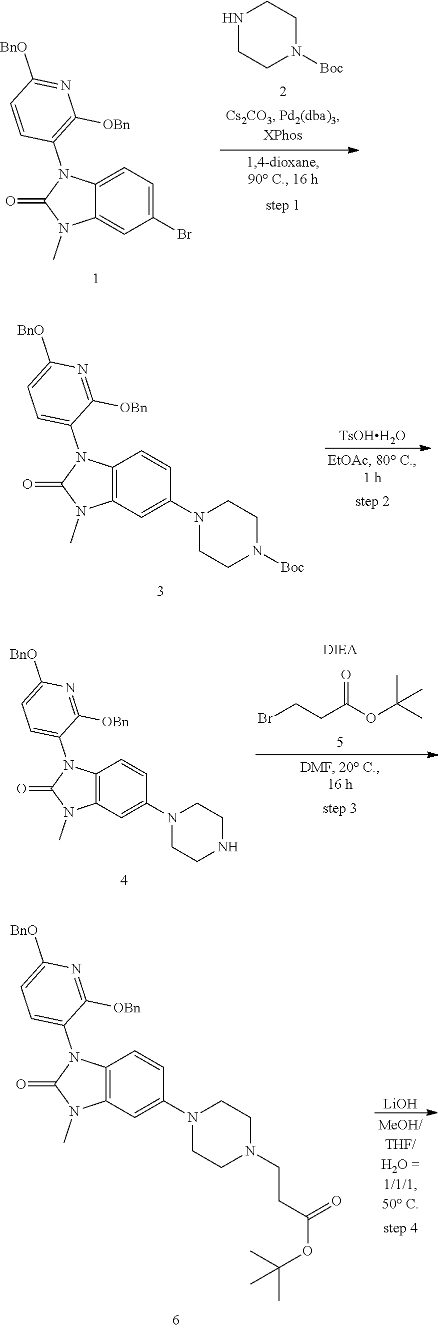

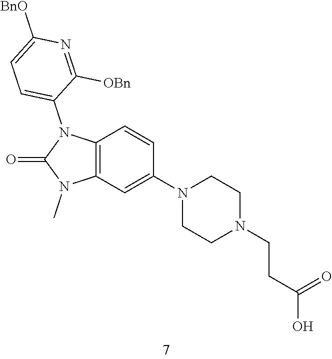 [SMILES: Cn1c(=O)n(-c2ccc(OCc3ccccc3)nc2OCc2ccccc2)c2ccc(N3CCN(CCC(=O)O)CC3)cc21]

### Step 1: tert-butyl 4-(1-(2,6-bis(benzyloxy)pyridin-3-yl)-3-methyl-2-oxo-2,3-dihydro-1H-benzo[d]imidazol-5-yl)piperazine-1-carboxylate (3)

To a mixture of 1-(2,6-bis(benzyloxy)pyridin-3-yl)-5-bromo-3-methyl-1H-benzo[d]imidazol-2 (3H)-one (1, 1 g, 1.94 mmol), tert-butyl piperazine-1-carboxylate (2, 396.75 mg, 2.13 mmol), Pd₂(dba)₃ (177.33 mg, 193.65 μmol), XPhos (184.64 mg, 387.31 μmol) and Cs₂CO₃ (1.89 g, 5.81 mmol) in dioxane (10 mL) under N₂. The mixture was stirred at 90° C. for 12 hr under N₂. The reaction mixture was concentrated under vacuum. The residue was purified by flash silica gel chromatography (flow: 50 mL/min; gradient: 0-100% ethylacetate in petroleum ether; ISCO®; 40 g SepaFlash®Silica Flash Column; ethylacetate/petroleumether=1/0) to afford tert-butyl 4-(1-(2,6-bis(benzyloxy)pyridin-3-yl)-3-methyl-2-oxo-2,3-dihydro-1H-benzo[d]imidazol-5-yl)piperazine-1-carboxylate (3, 1.1 g, 1.72 mmol, 89% yield) as a yellow solid.

LCMS (ESI): m/z 622.3 [M+H]⁺

### Step 2: 1-(2,6-bis(benzyloxy)pyridin-3-yl)-3-methyl-5-(piperazin-1-yl)-1H-benzo[d]imidazol-2 (3H)-one (4)

To a mixture of tert-butyl 4-[1-(2,6-dibenzyloxy-3-pyridyl)-3-methyl-2-oxo-benzimidazol-5-yl]piperazine-1-carboxylate (3, 500 mg, 804.22 μmol) in EtOAc (5 mL) was p-toluene sulfonic acid monohydrate (305.95 mg, 1.61 mmol, 246.73 uL). The mixture was stirred at 80° C. for 1 h. The reaction mixture was concentrated to get a residue. The residue was purified by reversed phase HPLC (FA) and then lyophilization. The 1-(2,6-bis(benzyloxy)pyridin-3-yl)-3-methyl-5-(piperazin-1-yl)-1H-benzo[d]imidazol-2 (3H)-one (4, 400 mg, 704.68 μmol, 88% yield, formic acid salt) as yellow solid LCMS (ESI): m/z 522.3 [M+H]⁺

### Step 3: tert-butyl 3-(4-(1-(2,6-bis(benzyloxy)pyridin-3-yl)-3-methyl-2-oxo-2,3-dihydro-1H-benzo[d]imidazol-5-yl)piperazin-1-yl)propanoate (6)

To a mixture of 1-(2,6-dibenzyloxy-3-pyridyl)-3-methyl-5-piperazin-1-yl-benzimidazol-2-one (4, 400 mg, 704.68 μmol, formic acid salt) and tert-butyl 3-bromopropanoate (5, 221.00 mg, 1.06 mmol) in DMF (4 mL) was added DIPEA (455.37 mg, 3.52 mmol, 613.71 uL). The mixture was stirred at 15° C. for 16 h. The reaction mixture was quenched by water (10 mL) and extracted with ethyl acetate (10 mL×3). The combined organic layers were washed with brine (10 mL), dried by anhydrous Na₂SO₄, filtered and concentrated in vacuum. The tert-butyl 3-(4-(1-(2,6-bis(benzyloxy)pyridin-3-yl)-3-methyl-2-oxo-2,3-dihydro-1H-benzo[d]imidazol-5-yl)piperazin-1-yl)propanoate (6, 600 mg, 692.55 μmol, 98% yield) as yellow solid LCMS (ESI): m/z 650.3 [M+H]⁺

### Step 4: 3-(4-(1-(2,6-bis(benzyloxy)pyridin-3-yl)-3-methyl-2-oxo-2,3-dihydro-1H-benzo[d]imidazol-5-yl)piperazin-1-yl)propanoic acid (7)

To a solution of tert-butyl 3-[4-[1-(2,6-dibenzyloxy-3-pyridyl)-3-methyl-2-oxo-benzimidazol-5-yl]piperazin-1-yl]propanoate (6, 600 mg, 923.39 μmol) in methanol (2 mL) and THF (2 mL) was added a solution of LiOH·H₂O (581 mg, 13.85 mmol) in Water (2 mL). The mixture was stirred at 50° C. for 2 h. The reaction mixture was concentrated to get a mixture. The mixture was purified by reversed phase HPLC (NH₃H₂O) and then lyophilization. The 3-(4-(1-(2,6-bis(benzyloxy)pyridin-3-yl)-3-methyl-2-oxo-2,3-dihydro-1H-benzo[d]imidazol-5-yl)piperazin-1-yl)propanoic acid (7, 300 mg, 500.28 μmol, 54% yield) as white solid.

LCMS (ESI): m/z 594.5 [M+H]⁺

### 2-(4-((1-(2,6-bis(benzyloxy)pyridin-3-yl)-3-methyl-2-oxo-2,3-dihydro-1H-benzo[d]imidazol-5-yl)amino)-3-methylphenyl)propanoic acid (7)

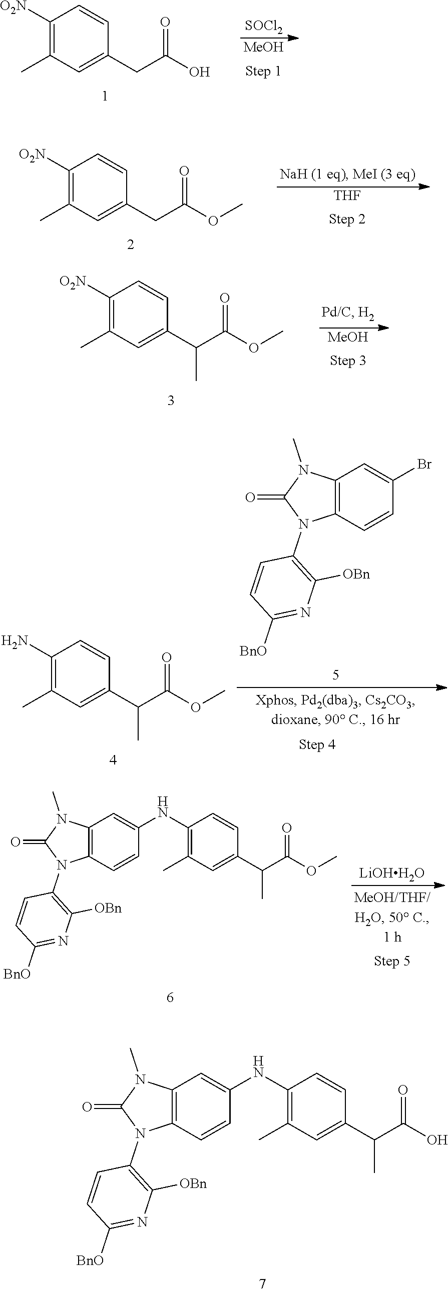

### Step 1: methyl 2-(3-methyl-4-nitrophenyl)acetate (2)

To a solution of 2-(3-methyl-4-nitrophenyl)acetic acid (1, 2 g, 10.25 mmol) in Methanol (20 mL) was cooled to 0° C. and added Thionylchloride (3.66 g, 30.74 mmol, 2.23 mL). Then the reaction mixture was stirred at 80° C. for 2 h. The mixture was concentrated to give methyl 2-(3-methyl-4-nitrophenyl)acetate (2, 2.1 g, 9.84 mmol, 96% yield) as yellow solid.

LCMS (ESI): m/z 210.1 [M+H]⁺

### Step 2: methyl 2-(3-methyl-4-nitrophenyl)propanoate (3)

To a solution of methyl 2-(3-methyl-4-nitrophenyl)acetate (2, 1 g, 4.78 mmol) in THF (10 mL) was added NaH (191.19 mg, 4.78 mmol, 60% purity) at 0° C. The mixture was stirred at 10° C. for 0.5 h. Then iodomethane (2.04 g, 14.34 mmol, 892.75 uL) was added. The reaction mixture was stirred at 10° C. for another 0.5 h. The reaction was quenched by water (10 mL), extracted with EtOAc (50 mL*3) and concentrated to get a residue. The residue was purified by flash silica gel chromatography (flow: 50 mL/min; gradient: 0-100% ethylacetate in petroleum ether; ISCO®; 40 g SepaFlash®Silica Flash Column) to afford methyl 2-(3-methyl-4-nitrophenyl)propanoate (3, 700 mg, 3.01 mmol, 63% yield) as yellow solid.

LCMS (ESI): m/z 224.1 [M+H]⁺

### Step 3: methyl 2-(4-amino-3-methylphenyl)propanoate (4)

To a solution of methyl 2-(3-methyl-4-nitro-phenyl)propanoate (3, 700 mg, 3.14 mmol) in Methanol (10 mL) was added Pd/C (100 mg, 10% purity). The suspension was degassed and purged with H₂ 3 times. The mixture was stirred under H₂ (15 psi) at 30° C. for 2 h. The reaction was filtered and concentrated to get methyl 2-(4-amino-3-methylphenyl)propanoate (4, 530 mg, 2.69 mmol, 86% yield) as yellow oil

LCMS (ESI): m/z 194.1 [M+H]⁺

### Step 4: methyl 2-(4-((1-(2,6-bis(benzyloxy)pyridin-3-yl)-3-methyl-2-oxo-2,3-dihydro-1H-benzo[d]imidazol-5-yl)amino)-3-methylphenyl)propanoate (6)

To a solution of 1-(2,6-bis(benzyloxy)pyridin-3-yl)-5-bromo-3-methyl-1H-benzo[d]imidazol-2 (3H)-one (5, 1.2 g, 2.32 mmol), methyl 2-(4-amino-3-methylphenyl)propanoate (4, 530 mg, 2.74 mmol) in dioxane (15 mL) was added Pd₂(dba)₃ (251.15 mg, 274.27 μmol) and Cs₂CO₃ (2.23 g, 6.86 mmol). The mixture was stirred at 90° C. for 16 h under N₂. The reaction mixture was filtered and concentrated under reduced pressure. The residue was purified by column chromatography (SiO₂, Petroleumether:Ethyl acetate=1:0 to 1:1) to afford methyl 2-(4-((1-(2,6-bis(benzyloxy)pyridin-3-yl)-3-methyl-2-oxo-2,3-dihydro-1H-benzo[d]imidazol-5-yl)amino)-3-methylphenyl)propanoate (6, 1.3 g, 2.01 mmol, 86% yield) as yellow solid.

LCMS (ESI): m/z 629.2 [M+H]⁺

### Step 5: 2-(4-((1-(2,6-bis(benzyloxy)pyridin-3-yl)-3-methyl-2-oxo-2,3-dihydro-1H-benzo[d]imidazol-5-yl)amino)-3-methylphenyl)propanoic acid (7)

To a solution of methyl 2-[4-[[1-(2,6-dibenzyloxy-3-pyridyl)-3-methyl-2-oxo-benzimidazol-5-yl]amino]-3-methyl-phenyl]propanoate (6, 300 mg, 477.16 μmol) in THF (2 mL) and MeOH (2 mL) was added a solution of LiOH·H₂O (60.07 mg, 1.43 mmol) in Water (2 mL). The mixture was stirred at 25° C. for 2 h. The reaction was quenched by 1N HCl and adjust pH to 6˜7 then concentrated to get a residue. The residue was purified by reversed phase HPLC (FA) and then lyophilization. The 2-(4-((1-(2,6-bis(benzyloxy)pyridin-3-yl)-3-methyl-2-oxo-2,3-dihydro-1H-benzo[d]imidazol-5-yl)amino)-3-methylphenyl)propanoic acid (7, 150 mg, 231.82 μmol, 49% yield) as yellow solid.

LCMS (ESI): m/z 615.3 [M+H]⁺

### 2-(5-(1-(2,6-bis(benzyloxy)pyridin-3-yl)-3-methyl-2-oxo-2,3-dihydro-1H-benzo[d]imidazol-5-yl)-3-methyl-1H-indazol-1-yl)acetic acid (6)

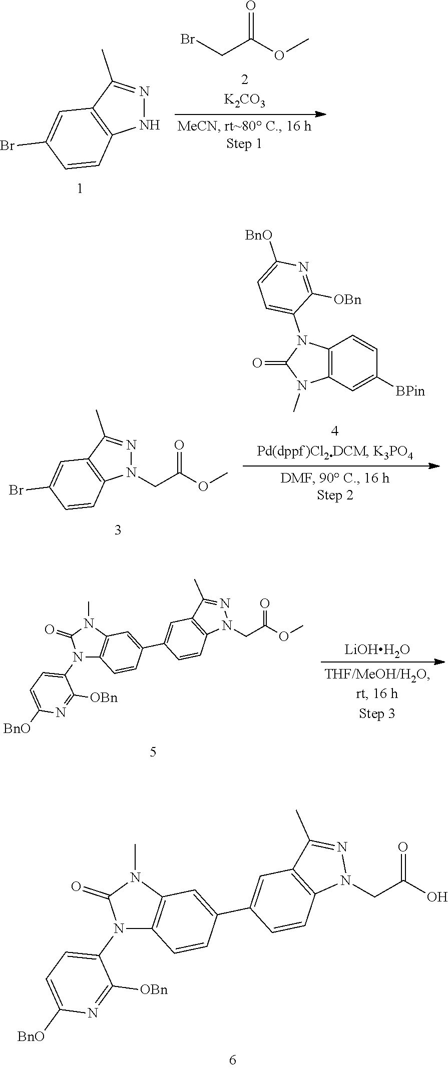

### Step 1: methyl 2-(5-bromo-3-methyl-1H-indazol-1-yl) acetate (3)

To a solution of 5-bromo-3-methyl-1H-indazole (1, 1 g, 4.74 mmol, 1 eq) and K₂CO₃ (1.96 g, 14.21 mmol, 857.84 μL, 3 eq) in MeCN (20 mL) was added methyl 2-bromoacetate (2, 869.75 mg, 5.69 mmol, 523.95 μL, 1.2 eq) at 20° C. The mixture was stirred at 80° C. for 16 h. The mixture was filtered and concentrated in vacuum. The residue was purified by flash silica gel chromatography (SiO₂, petroleum ether/ethyl acetate=1/0 to 4/1) to afford methyl 2-(5-bromo-3-methyl-1H-indazol-1-yl) acetate (3, 1 g, 3.50 mmol, 74% yield) as a white solid.

LCMS (ESI): m/z 283.0 [M+H]⁺

¹H NMR (400 MHz, CDCl3) δ 7.77-7.74 (m, 1H), 7.52 (d, J=9.2 Hz, 1H), 7.34 (dd, J=1.6, 9.2 Hz, 1H), 5.18 (s, 2H), 3.80 (s, 3H), 2.57 (s, 3H).

### Step 2: methyl 2-(5-(1-(2, 6-bis (benzyloxy) pyridin-3-yl)-3-methyl-2-oxo-2, 3-dihydro-1H-benzo[d]imidazol-5-yl)-3-methyl-1H-indazol-1-yl) acetate (5)

A mixture of methyl 2-(5-bromo-3-methyl-1H-indazol-1-yl)acetate (3, 0.6 g, 2.12 mmol), 1-(2,6-bis(benzyloxy)pyridin-3-yl)-3-methyl-5-(4,4,5,5-tetramethyl-1,3,2-dioxaborolan-2-yl)-1H-benzo[d]imidazol-2 (3H)-one (1.55 g, 2.76 mmol), K₃PO₄ (899.68 mg, 4.24 mmol), and cyclopentyl(diphenyl)phosphane; dichloropalladium; iron (155.07 mg, 211.92 μmol) in DMF (6 mL) was degassed and purged with 3 times, and then the mixture was stirred at 80° C. for 16 hr under N₂ atmosphere. The mixture was filtered and concentrated in vacuum. The residue was purified by flash silica gel chromatography (SiO₂, petroleum ether/ethyl acetate=1/0 to 0/1) to afford methyl 2-(5-(1-(2,6-bis(benzyloxy)pyridin-3-yl)-3-methyl-2-oxo-2,3-dihydro-1H-benzo[d]imidazol-5-yl)-3-methyl-1H-indazol-1-yl)acetate (5, 1.3 g, 1.85 mmol, 87% yield) as a yellow solid.

LCMS (ESI): m/z 640.3 [M+H]⁺

### Step 3: 2-(5-(1-(2,6-bis(benzyloxy)pyridin-3-yl)-3-methyl-2-oxo-2,3-dihydro-1H-benzo[d]imidazol-5-yl)-3-methyl-1H-indazol-1-yl)acetic acid (6)

To a solution of methyl 2-(5-(1-(2,6-bis(benzyloxy)pyridin-3-yl)-3-methyl-2-oxo-2,3-dihydro-1H-benzo[d]imidazol-5-yl)-3-methyl-1H-indazol-1-yl)acetate (5, 1.3 g, 2.03 mmol, 1 eq) in Methanol (10 mL), Water (10 mL) and THF (10 mL) was added LiOH·H₂O (426.39 mg, 10.16 mmol, 282.38 μL). The mixture was stirred at 20° C. for 16 h. The reaction mixture was adjusted pH to 3 with 1N HCl aqueous. The reaction mixture was poured into water (10 mL) and extracted with ethyl acetate (2×10 mL). Organic phases were combined and washed with brine (20 mL), dried by anhydrous Na₂SO₄, filtered and concentrated in vacuum. The residue was purified by flash silica gel chromatography (SiO₂, DCE/MeOH=1/0 to 0/1) to afford 2-[5-[1-(2,6-dibenzyloxy-3-pyridyl)-3-methyl-2-oxo-benzimidazol-5-yl]-3-methyl-indazol-1-yl]acetic acid (6, 1 g, 1.33 mmol, 65% yield) as a yellow solid.

LCMS (ESI): m/z 626.3 [M+H]⁺

### 2-((2-(1-(2,6-bis(benzyloxy)pyridin-3-yl)-3-methyl-2-oxo-2,3-dihydro-1H-benzo[d]imidazol-5-yl)-2-azaspiro[3.3]heptan-6-yl)oxy)acetic acid (7)

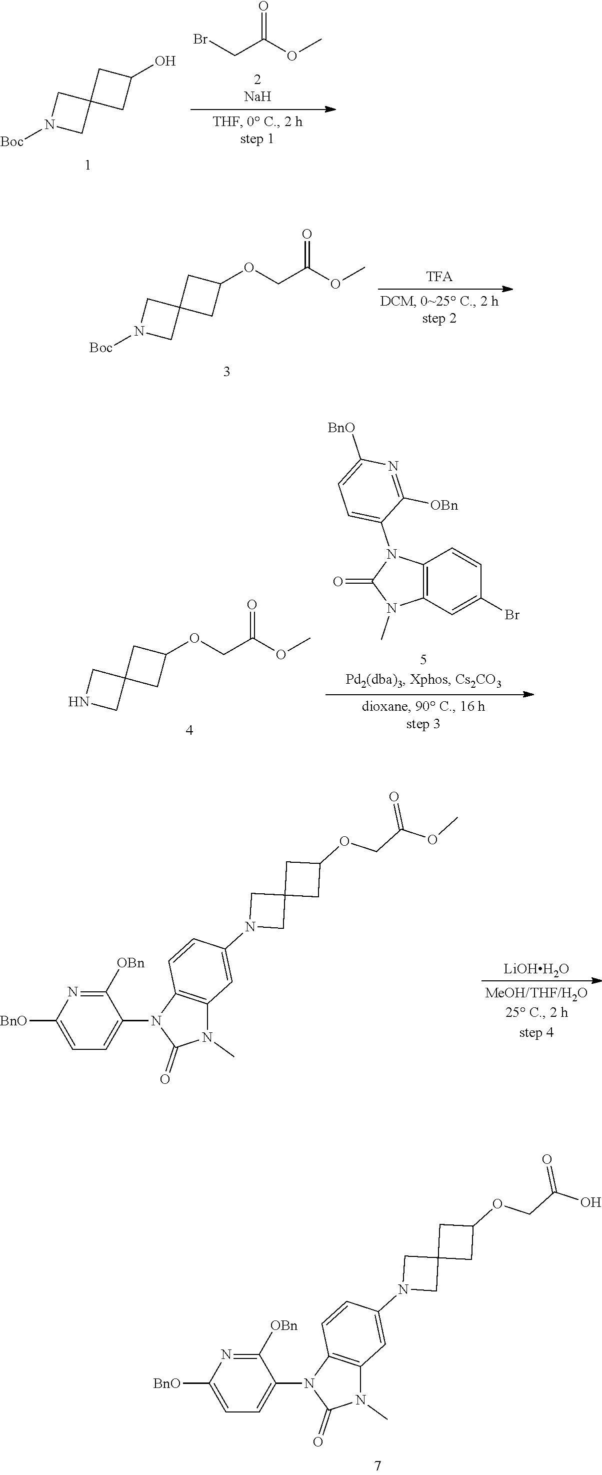

### Step 1: tert-butyl 6-(2-methoxy-2-oxoethoxy)-2-azaspiro[3.3]heptane-2-carboxylate (3)

To a solution of tert-butyl 6-hydroxy-2-azaspiro[3.3]heptane-2-carboxylate (1, 1 g, 4.69 mmol) and NaH (3.59 mg, 9.38 mmol, 60% purity) in THF (15 mL) was added methyl 2-bromoacetate (2, 860.73 mg, 5.63 mmol, 518.51 μL) at 0° C. Then the mixture was stirred at 20° C. for 1 h. The mixture was quenched with H₂O (10 mL), extracted with EA (20 mL*3). The combined organic layer was washed with brine (20 mL), dried over anhydrous Na₂SO₄, filtered and the filter was concentrated under vacuum. The residue was purified on automated flash chromatography system (ethyl acetate/petroleum ether from 0:1 to 1:0) and concentrated under vacuum. Compound tert-butyl 6-(2-methoxy-2-oxoethoxy)-2-azaspiro[3.3]heptane-2-carboxylate (3, 1 g, 3.50 mmol, 75% yield) as colorless oil.

¹H NMR (400 MHz, CDCl3) δ=4.04-3.94 (m, 3H), 3.88 (d, J=6.2 Hz, 4H), 3.76 (s, 3H), 2.50 (ddd, J=3.0, 6.8, 9.9 Hz, 2H), 2.27-2.12 (m, 2H), 1.43 (s, 9H).

### Step 2: methyl 2-(2-azaspiro[3.3]heptan-6-yloxy)acetate (4)

To a solution of tert-butyl 6-(2-methoxy-2-oxoethoxy)-2-azaspiro[3.3]heptane-2-carboxylate (3, 1 g, 3.50 mmol, 1 eq) in DCM (12 mL) was added 2,2,2-trifluoroacetic acid (4.44 g, 38.94 mmol, 3 mL) at 0° C. under N₂. The reaction mixture was stirred at 15° C. for 2 h. The reaction mixture was concentrated under vacuum to remove DCM and added H₂O (20 mL) for lyophilization. The residue was added toluene (20 mL×2) and concentrated under vacuum to afford methyl 2-(2-azaspiro[3.3]heptan-6-yloxy)acetate (4, 1 g, 3.34 mmol, 115% yield, TFA salt) as a yellow oil. The crude product was used in the next step without further purification.

¹H NMR (400 MHz, CDCl3) δ=9.24 (br s, 2H), 4.08 (br t, J=5.6 Hz, 4H), 4.03-3.91 (m, 3H), 3.76 (s, 3H), 2.74-2.55 (m, 2H), 2.34-2.19 (m, 2H).

### Step 3: methyl 2-((2-(1-(2,6-bis(benzyloxy)pyridin-3-yl)-3-methyl-2-oxo-2,3-dihydro-1H-benzo[d]imidazol-5-yl)-2-azaspiro[3.3]heptan-6-yl)oxy)acetate (6)

To a solution of 1-(2,6-bis(benzyloxy)pyridin-3-yl)-5-bromo-3-methyl-1H-benzo[d]imidazol-2 (3H)-one (5, 1.19 g, 2.30 mmol, TFA salt) and methyl 2-(2-azaspiro[3.3]heptan-6-yloxy)acetate (4, 896.48 mg, 3.00 mmol, TFA salt) in dioxane (12 mL) was added (1E,4E)-1,5-diphenylpenta-1,4-dien-3-one; palladium (211.03 mg, 230.45 μmol), dicyclohexyl-[2-(2,4,6-triisopropylphenyl)phenyl]phosphane (219.72 mg, 460.90 μmol) and dicesium; carbonate (2.25 g, 6.91 mmol). The reaction was stirred at 90° C. for 16 hrs under N₂. The reaction was diluted with water (20 mL) and extracted with EA (30 mL*3). The combined organic phase was dried by anhydrous Na₂SO₄ and concentrated under vacuum. The residue was purified by column chromatography on silica gel (petroleum ether/ethyl acetate=1/1) and concentrated under vacuum. Compound methyl 2-((2-(1-(2,6-bis(benzyloxy)pyridin-3-yl)-3-methyl-2-oxo-2,3-dihydro-1H-benzo[d]imidazol-5-yl)-2-azaspiro[3.3]heptan-6-yl)oxy)acetate (6, 0.23 g, 370.55 μmol, 16% yield) as yellow solid.

¹H NMR (400 MHz, DMSO-d6) δ=7.74 (d, J=8.2 Hz, 1H), 7.46-7.34 (m, 5H), 7.31-7.22 (m, 5H), 6.58 (d, J=8.3 Hz, 1H), 6.48 (d, J=8.3 Hz, 1H), 6.29 (d, J=2.0 Hz, 1H), 6.03 (dd, J=2.0, 8.4 Hz, 1H), 5.43-5.27 (m, 4H), 4.05-4.01 (m, 3H), 3.73 (d, J=10.9 Hz, 4H), 3.67-3.62 (m, 3H), 3.33-3.32 (m, 3H), 2.48-2.42 (m, 2H), 2.11 (ddd, J=2.8, 7.1, 9.9 Hz, 2H).

### Step 4: 2-((2-(1-(2,6-bis(benzyloxy)pyridin-3-yl)-3-methyl-2-oxo-2,3-dihydro-1H-benzo[d]imidazol-5-yl)-2-azaspiro[3.3]heptan-6-yl)oxy)acetic acid (7)

To a solution of methyl 2-((2-(1-(2,6-bis(benzyloxy)pyridin-3-yl)-3-methyl-2-oxo-2,3-dihydro-1H-benzo[d]imidazol-5-yl)-2-azaspiro[3.3]heptan-6-yl)oxy)acetate (6, 0.25 g, 402.78 μmol) in THF (1 mL), Methanol (2 mL) and Water (1 mL) was added lithium hydroxide hydrate (84.51 mg, 2.01 mmol, 55.97 uL). The reaction was stirred at 30° C. for 2 hrs. The reaction mixture was concentrated to remove THF and MeOH, adjusted to pH=4 by 1N HCl solution and extracted with EA (20 mL*3). The combined with organic phase was dried by anhydrous Na₂SO₄. The residue was purified by reversed-phase column (0.1% FA). The desired fraction was collected and dried by lyophilization. Compound 2-((2-(1-(2,6-bis(benzyloxy)pyridin-3-yl)-3-methyl-2-oxo-2,3-dihydro-1H-benzo[d]imidazol-5-yl)-2-azaspiro[3.3]heptan-6-yl)oxy)acetic acid (7, 0.2 g, 306.42 μmol, 76% yield, formic acid salt) as white solid.

¹H NMR (400 MHz, DMSO-d6) δ=8.27 (s, 1H), 7.74 (d, J=8.3 Hz, 1H), 7.46-7.32 (m, 5H), 7.31-7.21 (m, 5H), 6.58 (d, J=8.3 Hz, 1H), 6.48 (d, J=8.3 Hz, 1H), 6.29 (d, J=2.0 Hz, 1H), 6.03 (dd, J=2.1, 8.4 Hz, 1H), 5.44-5.25 (m, 4H), 4.01 (quin, J=7.0 Hz, 1H), 3.80 (s, 2H), 3.75 (s, 2H), 3.71 (s, 2H), 3.31 (s, 3H), 2.48-2.41 (m, 2H), 2.15-2.05 (m, 2H).

### 2-(1-(1-(2,6-bis(benzyloxy)pyridin-3-yl)-3-methyl-2-oxo-2,3-dihydro-1H-benzo[d]imidazol-5-yl)azepan-4-yl)acetic acid (7)

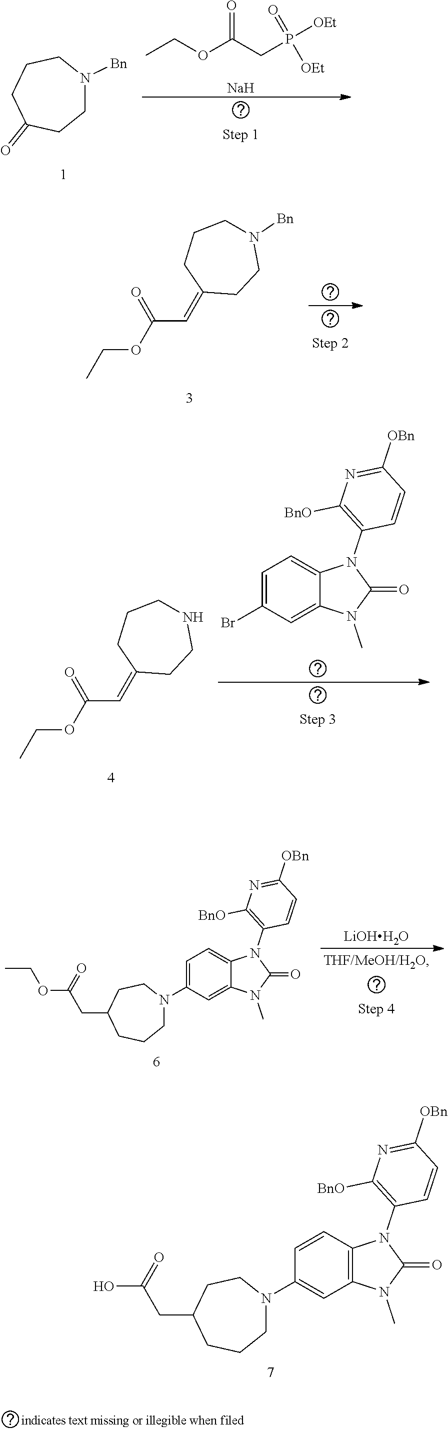

### Step 1: ethyl 2-(1-benzylazepan-4-ylidene)acetate (3)

To a solution of NaH (2.13 g 49.19 mmol, 60% purity) in DME (50 mL) was added ethyl 2-(diethoxyphosphoryl)acetate (2, 11.03 g, 49.19 mmol, 9.76 mL) under 0° C. The mixture was stirred at 20° C. for 30 min. A solution of 1-benzylazepan-4-one (1, 5 g, 24.60 mmol) in DME (50 mL) was added to the mixture at 0° C., the mixture was stirred at 20° C. for 15.5 h under N₂ atmosphere. The residue was diluted with H₂O (300 mL) and extracted with ethyl acetate (100 mL×3). The combined organic layers were washed with brine (150 mL×3), dried over Na₂SO₄, filtered and concentrated under reduced pressure to afford ethyl 2-(1-benzylazepan-4-ylidene)acetate (3, 5.1 g, 17.33 mmol, 70% yield) as a white solid.

LCMS (ESI): m/z 274.2 [M+H]⁺

### Step 2: ethyl 2-(azepan-4-yl)acetate (4)

To a solution of ethyl 2-(1-benzylazepan-4-ylidene)acetate (3, 5 g, 18.29 mmol) in EtOH (50 mL) was added Pd(OH)₂/C (770 mg, 10% purity). The mixture was stirred at 20° C. for 16 h under H₂ (15 Psi). The reaction mixture was filtered and concentrated under reduced pressure to afford ethyl 2-(azepan-4-yl)acetate (4, 3.5 g, 17.30 mmol, 94% yield) as a white solid.

¹H NMR (400 MHz, DMSO-d₆) δ=4.08-4.03 (m, 2H), 2.83-2.73 (m, 2H), 2.71-2.57 (m, 2H), 2.44 (br s, 1H), 2.22 (d, J=7.3 Hz, 2H), 2.05-1.90 (m, 1H), 1.75-1.55 (m, 3H), 1.52-1.39 (m, 1H), 1.19 (br d, J=7.4 Hz, 3H)

### Step 3: ethyl 2-(1-(1-(2,6-bis(benzyloxy)pyridin-3-yl)-3-methyl-2-oxo-2,3-dihydro-1H-benzo[d]imidazol-5-yl)azepan-4-yl)acetate (6)

To a solution of ethyl 2-(azepan-4-yl)acetate (4, 358.77 mg, 1.94 mmol) in dioxane (5 mL) was added 1-(2,6-bis(benzyloxy)pyridin-3-yl)-5-bromo-3-methyl-1H-benzo[d]imidazol-2 (3H)-one (5, 500 mg, 0.96 mmol), Cs₂CO₃ (946 mg, 2.90 mmol), Xphos (92.32 mg, 193.65 μmol) and Pd₂(dba)₃ (88.51 mg, 96.83 μmol). The mixture was degrees in vacuum and purged by N₂, then the mixture was stirred at 90° C. for 16 hrs under N₂ atmosphere. The reaction mixture was filtered and concentrated under reduced pressure. The residue was purified by column chromatography (SiO₂, Petroleum ether:Ethyl acetate=100:0 to 1:1) to afford ethyl 2-(1-(1-(2,6-bis(benzyloxy)pyridin-3-yl)-3-methyl-2-oxo-2,3-dihydro-1H-benzo[d]imidazol-5-yl)azepan-4-yl)acetate (6, 450 mg, 656.2 μmol, 68% yield) as a white solid.

LCMS (ESI): m/z 621.3 [M+H]⁺

### Step 4: 2-(1-(1-(2,6-bis(benzyloxy)pyridin-3-yl)-3-methyl-2-oxo-2,3-dihydro-1H-benzo[d]imidazol-5-yl)azepan-4-yl)acetic acid (7)

To a solution of ethyl 2-(1-(1-(2,6-bis(benzyloxy)pyridin-3-yl)-3-methyl-2-oxo-2,3-dihydro-1H-benzo[d]imidazol-5-yl)azepan-4-yl)acetate (6, 400 mg, 0.644 mmol) in H₂O (10 mL), MeOH (10 mL) and THF (10 mL) was added LiOH H₂O (270 mg, 6.44 mmol, 10 eq). The mixture was stirred at 60° C. for 2 h. The residue was diluted with H₂O (150 mL) and extracted with ethyl acetate (150 mL). The combined organic layers were washed with brine (100 mL), dried over Na₂SO₄, filtered and concentrated under reduced pressure to afford 2-(1-(1-(2,6-bis(benzyloxy)pyridin-3-yl)-3-methyl-2-oxo-2,3-dihydro-1H-benzo[d]imidazol-5-yl)azepan-4-yl)acetic acid (7, 350 mg, 0.574 mmol, 89% yield) as a white solid. LCMS (ESI): m/z 593.5 [M+H]⁺

### (1R,2S)-2-(4-(1-(2,6-bis(benzyloxy)pyridin-3-yl)-3-methyl-2-oxo-2,3-dihydro-1H-benzo[d]imidazol-5-yl)benzyl)cyclopropanecarboxylic acid (9)

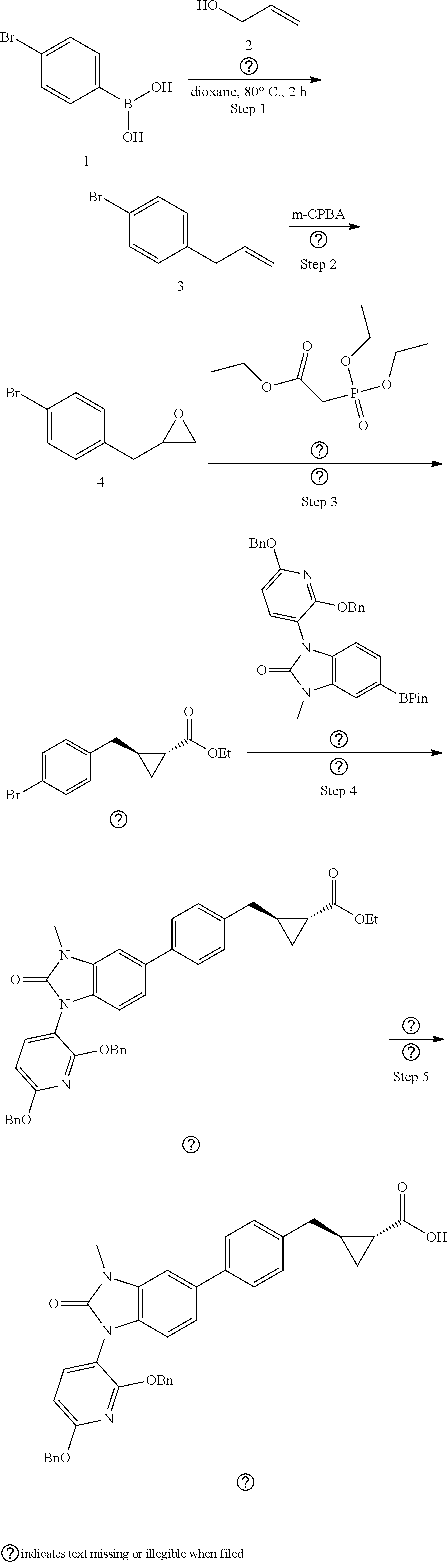

### Step 1:1-allyl-4-bromobenzene (3)

To a solution of (4-bromophenyl)boronic acid (1, 10.37 g, 51.65 mmol) and prop-2-en-1-ol (2.5 g, 43.04 mmol, 2.93 mL) in dioxane (50 mL) was added (1E,4E)-1,5-diphenylpenta-1,4-dien-3-one; palladium (788.34 mg, 860.90 μmol) and triphenyl phosphite (267.12 mg, 860.90 μmol).

The mixture was stirred at 80° C. for 2 hrs under N₂. The mixture was diluted with water (100 mL) and extracted with EtOAc (100 mL*2). The organic phase was washed with brine (100 mL), dried with anhydrous Na₂SO₄, filtered and concentrated under vacuum. The residue was purified by column chromatography (SiO₂, Petroleum ether/Ethyl acetate=1/0 to 20/1) and concentrated under vacuum to afford 1-allyl-4-bromobenzene (3, 7.0 g, 31.97 mmol, 74% yield) as colorless oil.

¹H NMR (400 MHz, CDCl3) δ=7.47-7.40 (m, 2H), 7.12-7.04 (m, 2H), 6.01-5.92 (m, 1H), 5.18-5.05 (m, 2H), 3.37 (d, J=6.4 Hz, 2H).

### Step 2: 2-(4-bromobenzyl)oxirane (4)

To a solution of 1-allyl-4-bromo-benzene (3, 3.0 g, 15.22 mmol) in DCM (80 mL) was added m-CPBA (3.94 g, 18.27 mmol, 80% purity) at 0° C., the mixture was stirred at 15° C. for 16 h. The mixture was poured into water (100 mL). The mixture was extracted with DCM (100 mL*3). The organic phase was washed with Na₂S₂O₃ (aq., 50 mL*2), dried with anhydrous Na₂SO₄, filtered and concentrated in vacuum. The residue was purified by column chromatography (SiO₂, Petroleum ether/Ethyl acetate=1/0 to 20/1) and concentrated under vacuum. Compound 2-(4-bromobenzyl)oxirane (4, 3.0 g, 12.67 mmol, 83% yield) as yellow oil.

¹H NMR (400 MHz, CDCl₃) δ=7.49-7.41 (m, 2H), 7.14 (d, J=8.3 Hz, 2H), 3.18-3.10 (m, 1H), 2.88-2.77 (m, 3H), 2.53 (dd, J=2.7, 5.0 Hz, 1H)

### Step 3: (1R,2S)-ethyl 2-(4-bromobenzyl)cyclopropanecarboxylate (6)

To a solution of ethyl 2-(diethoxyphosphoryl)acetate (5, 3.16 g, 14.08 mmol, 2.79 mL) in toluene (15 mL) was added NaH (563.14 mg, 14.08 mmol, 60% purity) at 0° C., the mixture was stirred at 0° C. for 0.5 h. 2-(4-bromobenzyl)oxirane (4, 1.5 g, 7.04 mmol) was added to the mixture at 0° C., the mixture was stirred at 105° C. for 16 h under N₂. Then NaH (281.57 mg, 7.04 mmol, 60% purity) was added at 0° C., the mixture was stirred at 105° C. for 3 h under N₂. The mixture was poured into ice aq·HCl (50 mL, 1M). The mixture was extracted with EtOAc (20 mL). The organic phase was washed with brine (50 mL), dried with anhydrous Na₂SO₄, filtered and concentrated in vacuum. The residue was purified by column chromatography (SiO₂, Petroleum ether/Ethyl acetate=100/1 to 10/1) to afford (1R,2S)-ethyl 2-(4-bromobenzyl)cyclopropanecarboxylate (6, 0.7 g, 1.98 mmol, 28% yield) as yellow oil.

¹H NMR (400 MHz, CDCl3) δ=7.48-7.40 (m, 2H), 7.11 (br d, J=6.8 Hz, 2H), 4.14 (q, J=7.0 Hz, 2H), 2.75-2.65 (m, 1H), 2.62-2.52 (m, 1H), 1.71-1.61 (m, 1H), 1.54-1.48 (m, 1H), 1.32-1.21 (m, 4H), 0.88-0.79 (m, 1H).

### Step 4: (1R,2S)-ethyl 2-(4-(1-(2,6-bis(benzyloxy)pyridin-3-yl)-3-methyl-2-oxo-2,3-dihydro-1H-benzo[d]imidazol-5-yl)benzyl)cyclopropanecarboxylate (8)

To a mixture of 1-(2,6-bis(benzyloxy)pyridin-3-yl)-3-methyl-5-(4,4,5,5-tetramethyl-1,3,2-dioxaborolan-2-yl)-1H-benzo[d]imidazol-2 (3H)-one (7, 500 mg, 887.39 μmol), (1R,2S)-ethyl 2-(4-bromobenzyl)cyclopropanecarboxylate (6, 251.27 mg, 887.39 μmol) and cataCXium A Pd G3 (64.72 mg, 88.74 μmol) in DMF (10 mL) was added K₃PO₄ (565.09 mg, 2.66 mmol) under N₂, the mixture was stirred at 90° C. for 16 h under N₂. The mixture was diluted with water (30 mL) and extracted with EtOAc (30 mL*3). The organic layer was concentrated under vacuum. The residue was purified by column chromatography (SiO₂, Petroleum ether/Ethyl acetate=30/1 to 1/1) to afford (1R,2S)-ethyl 2-(4-(1-(2,6-bis(benzyloxy)pyridin-3-yl)-3-methyl-2-oxo-2,3-dihydro-1H-benzo[d]imidazol-5-yl)benzyl)cyclopropanecarboxylate (8, 0.5 g, 577.58 μmol, 65% yield) as yellow solid.

LCMS (ESI): m/z 640.2 [M+H]⁺

### Step 5: (1R,2S)-2-(4-(1-(2,6-bis(benzyloxy)pyridin-3-yl)-3-methyl-2-oxo-2,3-dihydro-1H-benzo[d]imidazol-5-yl)benzyl)cyclopropanecarboxylic acid (9)

To a mixture of ethyl (1R,2S)-2-[[4-[1-(2,6-dibenzyloxy-3-pyridyl)-3-methyl-2-oxo-benzimidazol-5-yl]phenyl]methyl]cyclopropanecarboxylate (8, 0.5 g, 781.57 μmol) and LiOH·H₂O (327.97 mg, 7.82 mmol) in THF (2 mL), Methanol (2 mL) and Water (2 mL) was stirred at 20° C. for 16 h. The mixture was adjusted to pH˜4 by 1 N HCl. The mixture was extracted with EtOAc (30 mL). The organic phase was washed with brine (50 mL), dried with anhydrous Na₂SO₄, filtered and concentrated in vacuum to afford (1R,2S)-2-(4-(1-(2,6-bis(benzyloxy)pyridin-3-yl)-3-methyl-2-oxo-2,3-dihydro-1H-benzo[d]imidazol-5-yl)benzyl)cyclopropanecarboxylic acid (9, 0.3 g, 456.13 μmol, 58% yield) as yellow solid.

LCMS (ESI): m/z 612.2[M+H]⁺

### 2-(1-(1-(2,6-bis(benzyloxy)pyridin-3-yl)-3-methyl-2-oxo-2,3-dihydro-1H-benzo[d]imidazol-5-yl)-4-methoxypiperidin-4-yl)acetic acid

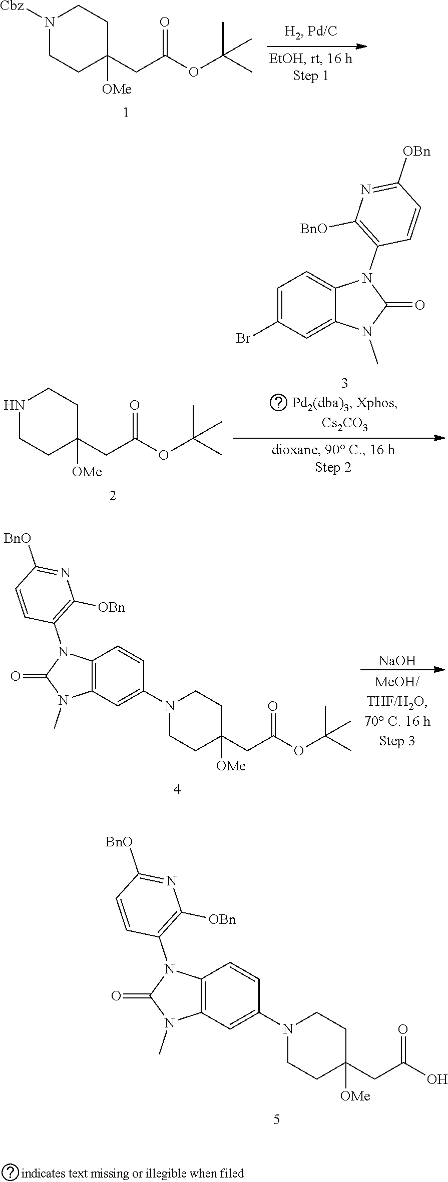 [SMILES: COC1(CC(=O)OC(C)(C)C)CCN(C(=O)OCc2ccccc2)CC1 ; Cn1c(=O)n(-c2ccc(OCc3ccccc3)nc2OCc2ccccc2)c2ccc(Br)cc21 ; COC1(CC(=O)OC(C)(C)C)CCNCC1 ; COC1(CC(=O)OC(C)(C)C)CCN(c2ccc3c(c2)n(C)c(=O)n3-c2ccc(OCc3ccccc3)nc2OCc2ccccc2)CC1 ; "NaOH" ; COC1(CC(=O)O)CCN(c2ccc3c(c2)n(C)c(=O)n3-c2ccc(OCc3ccccc3)nc2OCc2ccccc2)CC1]

### Step 1: tert-butyl 2-(4-methoxypiperidin-4-yl) acetate (2)

To a solution of benzyl 4-(2-(tert-butoxy)-2-oxoethyl)-4-methoxypiperidine-1-carboxylate (1, 1.9 g, 5.23 mmol) in Ethanol (200 mL) was added Pd/C (0.4 g, 10% purity). The suspension was purged with H₂ (3 times). The mixture was stirred under H₂ (15 psi) at 25° C. for 16 h. The mixture was filtered and concentrated under vacuum to afford tert-butyl 2-(4-methoxypiperidin-4-yl)acetate (2, 1.0 g, 4.36 mmol, 83% yield) as black oil.

¹H NMR (400 MHz, CDCl3) δ=3.26-3.23 (m, 3H), 2.98-2.86 (m, 2H), 2.84-2.75 (m, 2H), 2.41-2.38 (m, 2H), 1.84 (br d, J=13.2 Hz, 2H), 1.65-1.54 (m, 2H), 1.47-1.46 (m, 9H).

### Step 2: tert-butyl 2-(1-(1-(2,6-bis(benzyloxy)pyridin-3-yl)-3-methyl-2-oxo-2,3-dihydro-1H-benzo[d]imidazol-5-yl)-4-methoxypiperidin-4-yl)acetate (4)

To a solution of 1-(2,6-bis(benzyloxy)pyridin-3-yl)-5-bromo-3-methyl-1H-benzo[d]imidazol-2 (3H)-one (3, 1.5 g, 2.90 mmol) and tert-butyl 2-(4-methoxypiperidin-4-yl)acetate (2, 865.95 mg, 3.78 mmol) in dioxane (10 mL) was added (1E,4E)-1,5-diphenylpenta-1,4-dien-3-one; palladium (266.00 mg, 290.48 μmol), dicyclohexyl-[2-(2,4,6-triisopropylphenyl)phenyl]phosphane (276.95 mg, 580.96 μmol) and dicesium; carbonate (2.84 g, 8.71 mmol). The reaction was stirred at 90° C. for 16 hrs under N₂. The reaction mixture was diluted with water (20 mL) and extracted with EA (30 mL*3). The combined with organic phase was dried by anhydrous Na₂SO₄ filtered and concentrated under vacuum. The residue was purified on automated flash chromatography system (ethyl acetate/petroleum ether from 0:1 to 1:0) and concentrated under vacuum to afford tert-butyl 2-(1-(1-(2,6-bis(benzyloxy)pyridin-3-yl)-3-methyl-2-oxo-2,3-dihydro-1H-benzo[d]imidazol-5-yl)-4-methoxypiperidin-4-yl)acetate (4, 1.0 g, 1.50 mmol, 52% yield) as yellow solid.

¹H NMR (400 MHz, DMSO-d₆) δ=7.76 (d, J=8.2 Hz, 1H), 7.47-7.33 (m, 5H), 7.27 (s, 5H), 6.86 (s, 1H), 6.65-6.56 (m, 2H), 6.55-6.49 (m, 1H), 5.37 (br s, 4H), 3.27 (br s, 5H), 3.18 (s, 3H), 2.91 (br t, J=10.7 Hz, 2H), 2.44 (s, 2H), 1.91 (br d, J=13.3 Hz, 2H), 1.80-1.68 (m, 2H), 1.40 (s, 9H).

### Step 3: 2-(1-(1-(2,6-bis(benzyloxy)pyridin-3-yl)-3-methyl-2-oxo-2,3-dihydro-1H-benzo[d]imidazol-5-yl)-4-methoxypiperidin-4-yl)acetic acid (5)

To a solution of tert-butyl 2-(1-(1-(2,6-bis(benzyloxy)pyridin-3-yl)-3-methyl-2-oxo-2,3-dihydro-1H-benzo[d]imidazol-5-yl)-4-methoxypiperidin-4-yl)acetate (4, 0.5 g, 752.12 μmol) in THF (4 mL), Methanol (4 mL) and Water (4 mL) was added sodium; hydroxide (300.83 mg, 7.52 mmol). The mixture was stirred at 70° C. for 16 h. The reaction mixture was adjusted pH to 3 with 1N HCl aqueous, and extracted with DCM (2*30 mL). Organic phases were combined and washed with brine (30 mL), dried by anhydrous Na₂SO₄, filtered and concentrated in vacuum. The residue was purified by reversed phase flash (flow: 85 mL/min; gradient: from 0-40% MeCN in water (0.1% FA) over 30 min; column: 40 g Flash Column Welch Ultimate XB_C18 20-40 μm; 120 Å) to afford 2-(1-(1-(2,6-bis(benzyloxy)pyridin-3-yl)-3-methyl-2-oxo-2,3-dihydro-1H-benzo[d]imidazol-5-yl)-4-methoxypiperidin-4-yl)acetic acid (5, 0.15 g, 205.28 μmol, 27% yield, formic acid salt) as yellow solid.

LCMS (ESI): m/z 608.9[M+H]⁺

### 2-[4-[1-(2,6-dioxo-3-piperidyl)-3-methyl-indazol-5-yl]-1-piperidyl]acetic acid (3)

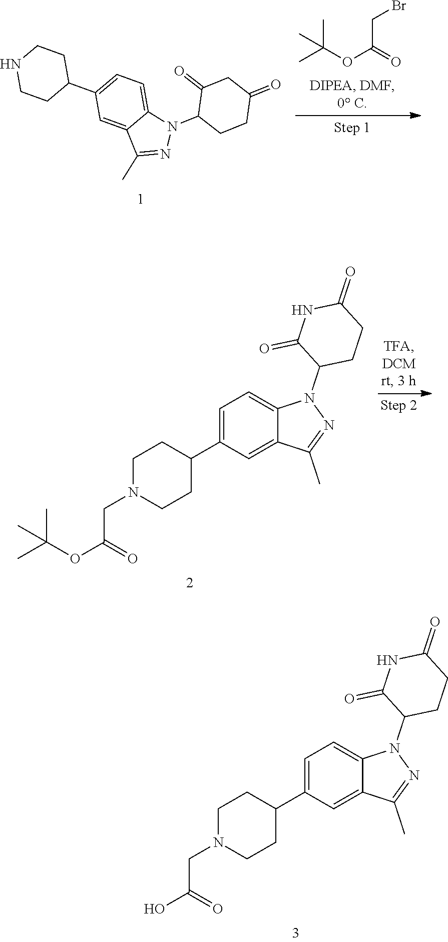

### Step 1: tert-butyl 2-[4-[1-(2,6-dioxo-3-piperidyl)-3-methyl-indazol-5-yl]-1-piperidyl]acetate (2)

Into a 20 mL vial containing a well-stirred solution of 3-[3-methyl-5-(4-piperidyl) indazol-1-yl]piperidine-2,6-dione (1, 100 mg, 170.29 μmol, TFA salt) in DMF (1 mL) were added DIPEA (66.03 mg, 510.88 μmol, 88.99 μL) and tert-butyl bromoacetate (29.89 mg, 153.26 μmol, 22.48 μL) at 0° C. The reaction mixture was stirred at ambient temperature for 30 min. After completion of the reaction, the reaction mixture was quenched with cold water (5 mL) at 0° C. The aqueous layer was extracted with ethylacetate (2×100 mL). The combined organic layer was dried over anhydrous Na₂SO₄, filtered and concentrated under reduced pressure. The crude compound was purified by reverse phase column chromatography [Silicycle C18 column; Mobile phase A: 0.1% formic acid in water; Mobile phase B: MeCN] to get tert-butyl 2-[4-[1-(2,6-dioxo-3-piperidyl)-3-methyl-indazol-5-yl]-1-piperidyl]acetate (2, 40 mg, 72.15 μmol, 42% yield, Formic acid salt) as an off-white solid.

LCMS (ES+): m/z 441.2 [M+H]⁺

### Step 2: 2-[4-[1-(2,6-dioxo-3-piperidyl)-3-methyl-indazol-5-yl]-1-piperidyl]acetic acid (3)

Into a 25 mL single neck round-bottomed flask containing a well-stirred solution of tert-butyl 2-[4-[1-(2,6-dioxo-3-piperidyl)-3-methyl-indazol-5-yl]-1-piperidyl]acetate (2, 150 mg, 259.59 μmol) in DCM (1.5 mL) was added TFA (591.99 mg, 5.19 mmol, 400.00 μL) and the reaction mixture was stirred at room temperature for 3 h. After completion of the reaction, the volatiles were removed under vacuum to dryness. The residue was washed with MTBE (50 mL) and dried to get 2-[4-[1-(2,6-dioxo-3-piperidyl)-3-methyl-indazol-5-yl]-1-piperidyl]acetic acid (3, 120 mg, 216.67 μmol, 83% yield, TFA salt) an off-white solid.

UP-LCMS (ES+): m/z 385.5 [M+H]⁺

### 2-[1-[1-(2,6-dioxo-3-piperidyl)-3-methyl-indazol-5-yl]-4-piperidyl]acetic acid (3)

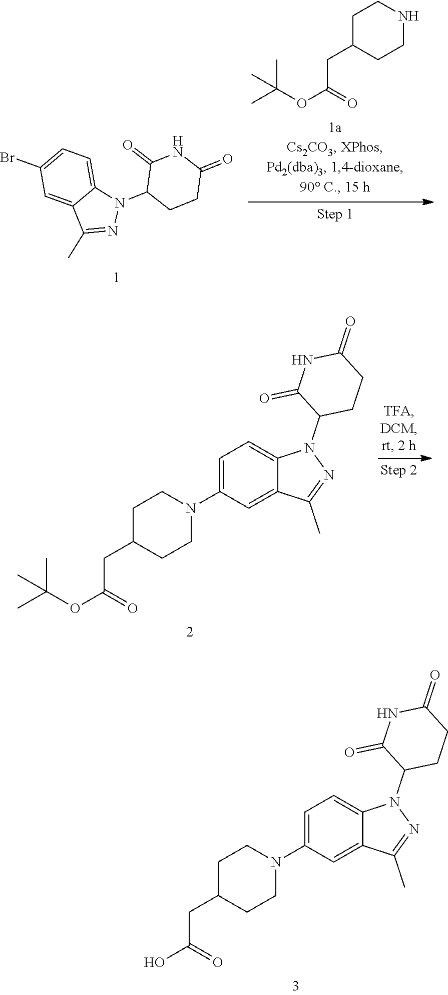 [SMILES: CC(C)(C)OC(=O)CC1CCNCC1 ; Cc1nn(C2CCC(=O)NC2=O)c2ccc(Br)cc12 ; Cc1nn(C2CCC(=O)NC2=O)c2ccc(N3CCC(CC(=O)OC(C)(C)C)CC3)cc12 ; Cc1nn(C2CCC(=O)NC2=O)c2ccc(N3CCC(CC(=O)O)CC3)cc12]

### Step 1: tert-butyl 2-[1-[1-(2,6-dioxo-3-piperidyl)-3-methyl-indazol-5-yl]-4-piperidyl]acetate (2)

Into a 15 mL pressure tube containing a well-stirred solution of 3-(5-bromo-3-methyl-indazol-1-yl)piperidine-2,6-dione (1, 200 mg, 620.82 μmol) in 1,4-dioxane (4 mL) were added tert-butyl 2-(4-piperidyl)acetate (1a, 160.84 mg, 807.06 μmol) and caesium carbonate (404.55 mg, 1.24 mmol). The reaction mixture was degassed by bubbling nitrogen gas for 10 min. Then, XPhos (29.60 mg, 62.08 μmol) and tris(dibenzylideneacetone)dipalladium(0) (113.70 mg, 124.16 μmol) were added. The reaction mixture was heated at 90° C. After 16 h, the reaction mixture was filtered through Celite and washed with ethylacetate (150 mL). The filtrate was concentrated under vacuum and the crude compound was purified by reverse phase column chromatography [Silicycle C18 column, Mobile phase A: 0.1% formic acid in water; Mobile phase B: MeCN] to get tert-butyl 2-[1-[1-(2,6-dioxo-3-piperidyl)-3-methyl-indazol-5-yl]-4-piperidyl]acetate (2, 30 mg, 50.45 μmol, 8% yield, formic acid salt) as an off-white solid.

LCMS (ES+): m/z 441.2 [M+H]⁺

### Step 2: 2-[1-[1-(2,6-dioxo-3-piperidyl)-3-methyl-indazol-5-yl]-4-piperidyl]acetic acid (3)

Into a 25 mL single neck round bottom flask containing a well-stirred solution of tert-butyl 2-[1-[1-(2,6-dioxo-3-piperidyl)-3-methyl-indazol-5-yl]-4-piperidyl]acetate (2, 70 mg, 88.67 μmol, Formic acid salt) in DCM (1 mL) was added TFA (10.11 mg, 88.67 μmol, 6.83 μL) and the reaction mixture was stirred at room temperature for 2 h. After completion of the reaction, the solvent was removed to dryness and the residue was washed with MTBE (50 mL) and dried to get 2-[1-[1-(2,6-dioxo-3-piperidyl)-3-methyl-indazol-5-yl]-4-piperidyl]acetic acid (3, 50 mg, 73.63 μmol, 83% yield, TFA salt) as a brown solid.

UP-LCMS (ES+): m/z 385.6 [M+H]⁺

### 3-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)cyclobutanecarboxylic acid (4)

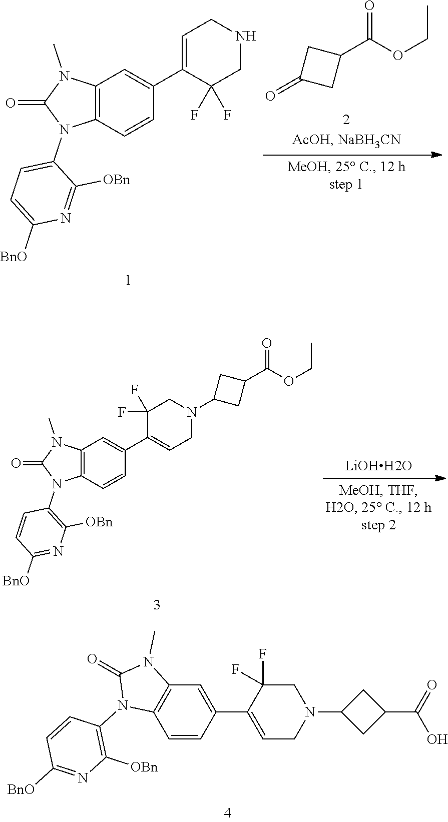

### Step 1: ethyl 3-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)cyclobutanecarboxylate (3)

To a solution of 1-(2,6-bis(benzyloxy)pyridin-3-yl)-5-(3,3-difluoro-1,2,3,6-tetrahydropyridin-4-yl)-3-methyl-1H-benzo[d]imidazol-2 (3H)-one (1, 650 mg, 1.17 mmol) ethyl 3-oxocyclobutanecarboxylate (2, 333.22 mg, 2.34 mmol) acetic acid (7.04 mg, 117.20 μmol, 6.70 μL) in methanol (6 mL) was added Sodium cyanoborohydride (220.96 mg, 3.52 mmol), the reaction mixture was stirred at 30° C. for 16 h. the reaction was purified by reversed phase (0.1% FA) to obtain ethyl 3-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)cyclobutanecarboxylate (3, 600 mg, 837.33 μmol, 71% yield) as a yellow solid LCMS (ESI): m/z 681.2 [M+H]⁺

### Step 2: 3-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)cyclobutanecarboxylic acid (4)

To a solution of ethyl 3-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)cyclobutanecarboxylate (3, 400 mg, 587.60 μmol) in THF (4 mL), Methanol (4 mL) was added lithium; hydroxide; hydrate (123.29 mg, 2.94 mmol, 81.65 μL) in Water (4 mL). The mixture was stirred at 20° C. for 2 h. After the reaction mixture was extracted with MTBE (3×50 mL), the water layer was poured into water (30 mL) and adjusted pH to 3 with citric acid (20 mL). Then the mixture was extracted with ethyl acetate (3×50 mL). Organic phases were combined and dried over anhydrous Na₂SO₄, filtered and concentrated in vacuum. The residue was purified by reversed phase (0.1% FA) to afford 3-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)cyclobutanecarboxylic acid (4, 260 mg, 378.44 μmol, 64% yield) as a yellow solid

LCMS (ESI): m/z 653.1 [M+H]⁺

### (R)-2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanoic acid (7A) and (S)-2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanoic acid (7B)

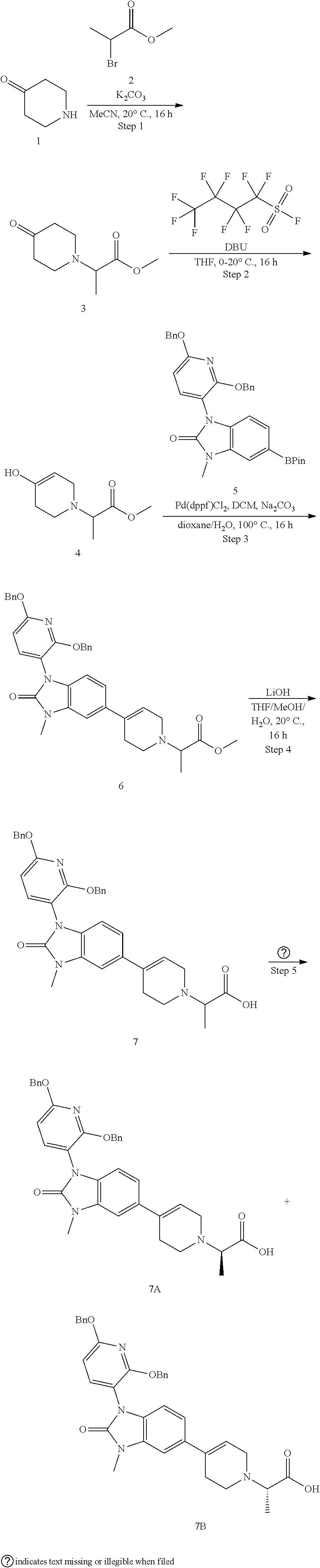

Note: Configurations are arbitrarily assigned.

### Step 1: methyl 2-(4-oxopiperidin-1-yl)propanoate (3)

A mixture of piperidin-4-one (1, 5 g, 36.88 mmol, HCl salt), methyl 2-bromopropanoate (2, 9.24 g, 55.31 mmol, 6.16 mL) in CH₃CN (50 mL) was added dipotassium; carbonate (15.29 g, 110.63 mmol, 6.68 mL) at 0° C., the reaction was stirred at 20° C. for 16 h. The mixture was filtered and concentrated in vacuum. The residue was purified by column chromatography (SiO₂, Petroleum ether/Ethyl acetate=1/0 to 0/1) to afford methyl 2-(4-oxopiperidin-1-yl)propanoate (3, 6 g, 29.15 mmol, 79% yield) as yellow oil

¹H NMR (400 MHz, CDCl3) δ=3.65 (s, 3H), 3.50-3.40 (m, 1H), 2.96-2.74 (m, 4H), 2.47-2.29 (m, 4H), 1.29 (d, J=7.1 Hz, 3H)

### Step 2: methyl 2-(4-(((perfluorobutyl)sulfonyl)oxy)-5,6-dihydropyridin-1 (2H)-yl)propanoate (4)

To a mixture of methyl 2-(4-oxopiperidin-1-yl)propanoate (3, 2 g, 10.80 mmol) and 2,3,4,6,7,8,9,10-octahydropyrimido[1,2-a]azepine (8.22 g, 53.99 mmol, 8.06 mL) in THF (20 mL) was added 1,1,2,2,3,3,4,4,4-nonafluorobutane-1-sulfonyl fluoride (16.31 g, 53.99 mmol, 9.32 mL) at 0° C., the mixture was stirred at 20° C. for 16 h under N₂. The mixture was poured into H₂O (500 mL). The mixture was extracted with EtOAc (200 mL×2). The organic phase was washed with brine (500 mL), dried with anhydrous Na₂SO₄, filtered and concentrated in vacuum. The residue was purified by column chromatography (SiO₂, Petroleum ether/Ethyl acetate=1/0 to 3/1) to afford methyl 2-(4-(((perfluorobutyl)sulfonyl)oxy)-5,6-dihydropyridin-1 (2H)-yl)propanoate (4, 3 g, 6.10 mmol, 56% yield) as yellow oil

¹H NMR (400 MHz, CDCl3) δ=5.68 (t, J=3.5 Hz, 1H), 3.64 (s, 3H), 3.42 (q, J=7.1 Hz, 1H), 3.33-3.22 (m, 2H), 2.87 (td, J=5.6, 11.6 Hz, 1H), 2.74 (td, J=5.6, 11.6 Hz, 1H), 2.38 (br d, J=1.7 Hz, 2H), 1.28 (d, J=7.1 Hz, 3H)

### Step 3: methyl 2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanoate (6)

To a mixture of 1-(2,6-bis(benzyloxy)pyridin-3-yl)-3-methyl-5-(4,4,5,5-tetramethyl-1,3,2-dioxaborolan-2-yl)-1H-benzo[d]imidazol-2 (3H)-one (5, 2.2 g, 3.90 mmol), methyl 2-(4-(((perfluorobutyl)sulfonyl)oxy)-5,6-dihydropyridin-1 (2H)-yl)propanoate (4, 2.01 g, 4.29 mmol) in dioxane (20 mL) Water (4 mL) was added cyclopentyl(diphenyl)phosphane; dichloromethane; dichloropalladium; iron (318.86 mg, 390.45 μmol), disodium; carbonate (1.24 g, 11.71 mmol, 490.71 μL) under N₂, the mixture was stirred at 100° C. for 16 h under N₂. The mixture was filtered and washed with EtOAc 100 mL. The organic phase was poured into H₂O (50 mL). The mixture was extracted with EtOAc (50 mL×3). The organic phase was dried with anhydrous Na₂SO₄, filtered and concentrated in vacuum. The residue was purified by reversed phase (0.1% FA) to afford methyl 2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanoate (6, 700 mg, 1.10 mmol, 28% yield) as yellow solid LCMS (ESI): m/z 605.1 [M+H]⁺

### Step 4: 2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanoic acid (7)

A mixture of methyl 2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanoate (6, 700 mg, 1.16 mmol) in THF (3 mL), Methanol (3 mL) and Water (3 mL) was added lithium; hydroxide; hydrate (485.78 mg, 11.58 mmol, 321.71 μL) and stirred at 20° C. for 16 h. the mixture was extracted with MBTE (3×50 mL). After the water layer was adjusted pH to 3 with aq citric acid (30 mL). Then the mixture was extracted with ethyl acetate (3×50 mL). Organic phases were dried over anhydrous Na₂SO₄, filtered and concentrated in vacuum to obtain 2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanoic acid (7, 500 mg, 804.18 μmol, 69% yield) as a yellow solid LCMS (ESI): m/z 591.2 [M+H]⁺

### Step 5: (R)-2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanoic acid (7A) and (S)-2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanoic acid (7B)

2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanoic acid (7, 500 mg, 846.50 μmol) was separated by SFC (Mobile Phase: 60% MeOH+ACN (0.1% NH₃H₂O) in Supercritical CO₂ Flow Rate: 70 g/min Cycle Time: 12.5 min, total time: 220 min Single injection volume: 4.0 ml Back Pressure: 100 bar to keep the CO₂ in Supercritical flow; column: Phenomenex-Cellulose-2 (250 mm×30 mm, 10 um) and further purification by reversed phase (0.1% FA) to afford (R)-2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanoic acid (7A, 140 mg, 225.17 μmol, 26.60% yield) as yellow solid and (S)-2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanoic acid (7B, 220 mg, 353.84 μmol, 42% yield) as yellow solid.

LCMS (ESI): m/z 591.3 [M+H]⁺

### (2S)-2-(1-(1-(2,6-dioxopiperidin-3-yl)-3-methyl-2-oxo-2,3-dihydro-1H-benzo[d]imidazol-5-yl)piperidin-4-yl)propanoic acid (8)

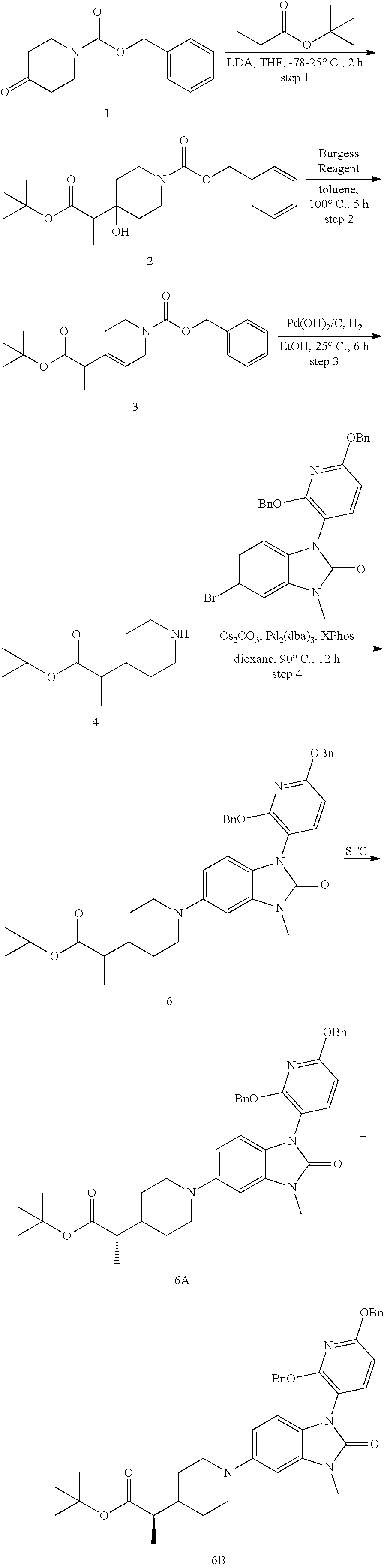

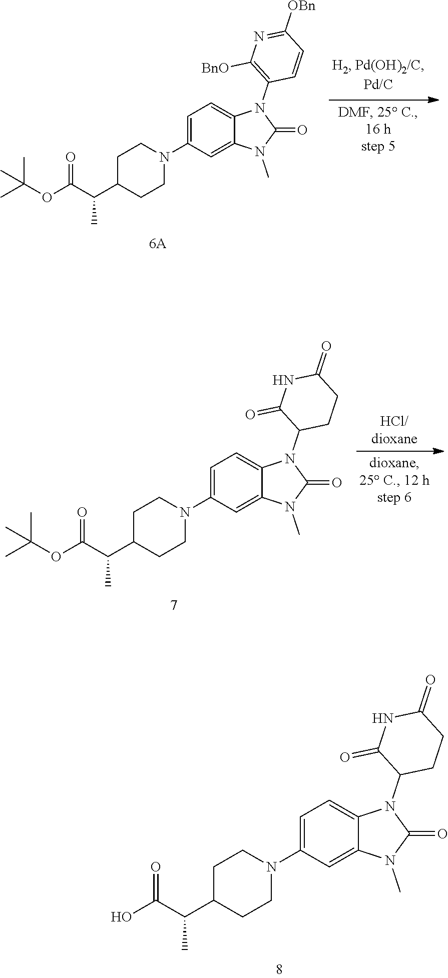 [SMILES: C[C@H](C(=O)OC(C)(C)C)C1CCN(c2ccc3c(c2)n(C)c(=O)n3-c2ccc(OCc3ccccc3)nc2OCc2ccccc2)CC1 ; C[C@H](C(=O)OC(C)(C)C)C1CCN(c2ccc3c(c2)n(C)c(=O)n3C2CCC(=O)NC2=O)CC1 ; C[C@H](C(=O)O)C1CCN(c2ccc3c(c2)n(C)c(=O)n3C2CCC(=O)NC2=O)CC1]

Note: Configurations are arbitrarily assigned.

### Step 1: benzyl 4-(1-(tert-butoxy)-1-oxopropan-2-yl)-4-hydroxypiperidine-1-carboxylate (2)

To a solution of (diisopropylamino)lithium (2 M, 16.08 mL) in THF (40 mL) was added tert-butyl propanoate (4.19 g, 32.15 mmol) in THF (40 mL) at −78° C. under N₂. After stirring at −78° C. for 1 h, a solution of benzyl 4-oxopiperidine-1-carboxylate (5 g, 21.44 mmol, 4.27 mL) in THF (40 mL) was added. The mixture was warmed to 25° C. and stirred for 15 h. The reaction mixture was poured into 1N HCl (50 mL) at 0° C., and then extracted with EtOAc (40 mL*2). The combined organic layers were washed with brine (50 mL*2), dried over Na₂SO₄, filtered and concentrated under reduced pressure. The crude was purified by flash silica gel chromatography (ISCO®; 330 g SepaFlash® Silica Flash Column, Eluent of 10%-35% EA/PE) and the eluent was concentrated under reduce pressure to afford benzyl 4-(2-tert-butoxy-1-methyl-2-oxo-ethyl)-4-hydroxy-piperidine-1-carboxylate (2, 6.5 g, 17.88 mmol, 83% yield) as yellow oil LCMS (ESI): m/z 264.4 [M−Boc+H]⁺

### Step 2: benzyl 4-(1-(tert-butoxy)-1-oxopropan-2-yl)-5,6-dihydropyridine-1 (2H)-carboxylate (3)

To a solution of benzyl 4-(2-tert-butoxy-1-methyl-2-oxo-ethyl)-4-hydroxy-piperidine-1-carboxylate (2, 6.5 g, 17.88 mmol) in toluene (180 mL) was added methoxycarbonyl-(triethylammonio)sulfonyl-azanide (6.39 g, 26.83 mmol). The mixture was stirred at 100° C. for 5 h. The reaction mixture was concentrated under reduced pressure to give the residue. The crude was purified by flash silica gel chromatography (ISCO®; 120 g SepaFlash® Silica Flash Column, Eluent of 5%-25% EA/PE) and the eluent was concentrated under reduce pressure to give benzyl 4-(2-tert-butoxy-1-methyl-2-oxo-ethyl)-3,6-dihydro-2H-pyridine-1-carboxylate (3, 5.2 g, 15.05 mmol, 84% yield) as a colorless oil.

¹H NMR (400 MHz, DMSO-d6) δ=7.37-7.31 (m, 5H), 5.53-5.51 (m, 1H), 5.08 (s, 2H), 3.98-3.79 (m, 2H), 3.53-3.40 (m, 2H), 3.03-2.99 (m, 1H), 2.14-1.92 (m, 2H), 1.37 (s, 9H), 1.12 (d, J=7.2 Hz, 3H)

### Step 3: tert-butyl 2-(piperidin-4-yl)propanoate (4)

To a solution of benzyl 4-(2-tert-butoxy-1-methyl-2-oxo-ethyl)-3,6-dihydro-2H-pyridine-1-carboxylate (3, 5.2 g, 15.05 mmol) in Ethanol (100 mL) was added Pd(OH)₂/C (1 g, 20% purity) under N₂ atmosphere. The suspension was degassed and purged with H2 3 times. The mixture was stirred under H₂ (15 Psi) at 30° C. for 3 hrs. The mixture was filtered through the Celite pad and the filter cake was washed with 3*20 mL EtOH. The combined filtrate was concentrated under vacuum and lyophilized to give tert-butyl 2-(piperidin-4-yl)propanoate (4, 3.2 g, 15.00 mmol, 100% yield) as colorless oil.

¹H NMR (400 MHz, CDCl3) δ=3.11-3.04 (m, 2H), 2.66-2.51 (m, 2H), 2.44 (br s, 1H), 2.13-2.09 (m, 1H), 1.72-1.53 (m, 3H), 1.43 (s, 9H), 1.30-1.09 (m, 2H), 1.05 (d, J=7.2 Hz, 3H).

### Step 4: tert-butyl 2-(1-(1-(2,6-bis (benzyloxy)pyri din-3-yl)-3-methyl-2-oxo-2,3-dihydro-1H-benzo[d]imidazol-5-yl)piperidin-4-yl)propanoate (6)

A mixture of 1-(2,6-bis(benzyloxy)pyridin-3-yl)-5-bromo-3-methyl-1H-benzo[d]imidazol-2 (3H)-one (3 g, 5.81 mmol), tert-butyl 2-(piperidin-4-yl)propanoate (4, 1.61 g, 7.55 mmol), (1E,4E)-1,5-diphenylpenta-1,4-dien-3-one; palladium (5, 532.00 mg, 580.96 μmol), dicyclohexyl-[2-(2,4,6-triisopropylphenyl)phenyl]phosphane (553.91 mg, 1.16 mmol) and dicesium; carbonate (3.79 g, 11.62 mmol) in dioxane (60 mL) was degassed and purged with N₂ 3 time, then the mixture was stirred at 90° C. for 12 h. The crude product was purified by column chromatography on silica gel eluted with petroleum ether/ethyl acetate (0/1 to 1/1). Then was purified by reverse column (H₂O/ACN=100/0 to 0/100, FA) to afford tert-butyl 2-(1-(1-(2,6-bis(benzyloxy)pyridin-3-yl)-3-methyl-2-oxo-2,3-dihydro-1H-benzo[d]imidazol-5-yl)piperidin-4-yl)propanoate (6, 1.3 g, 1.91 mmol, 32.85% yield) as yellow oil. The compound was separated by Chiral resolution for chiral SFC (“Column: Chiralpak AS-3 50×4.6 mm I.D., 3 um Mobile phase: Phase A for CO₂, and Phase B for MeOH (0.05% DEA). (S)-tert-butyl-2-(1-(1-(2,6-bis(benzyloxy)pyridin-3-yl)-3-methyl-2-oxo-2,3-dihydro-1H-benzo[d]imidazol-5-yl)piperidin-4-yl)propanoate (6A, 0.6 g, 915.10 μmol, 45.67% yield) as yellow oil. 2-(1-(1-(2, 6-bis(benzyloxy)pyridin-3-yl)-3-methyl-2-oxo-2,3-dihydro-1H-benzo[d]imidazol-5-yl)piperidin-4-yl)propanoate (6B, 0.4 g, 593.40 μmol, 30% yield) as yellow oil. The stereocenters of 6A and 6B were assigned arbitrarily.

LCMS (ESI): m/z 649.3 [M+H]⁺

### Step 5: (2S)-tert-butyl 2-(1-(1-(2,6-dioxopiperidin-3-yl)-3-methyl-2-oxo-2, 3-dihydro-1H-benzo[d]imidazol-5-yl)piperidin-4-yl)propanoate (7)

To a solution of (S)-tert-butyl 2-(1-(1-(2,6-bis(benzyloxy)pyridin-3-yl)-3-methyl-2-oxo-2,3-dihydro-1H-benzo[d]imidazol-5-yl)piperidin-4-yl)propanoate (6A, 0.13 g, 200.37 μmol) in DMF (12 mL) was added Pd/C (250 mg, 10% purity) and Pd(OH)₂/C (250 mg, 724.43 μmol, 10% purity). The mixture was stirred at 25° C. for 7 h under H₂ (15 Psi). The reaction mixture was filtered and concentrated under reduced pressure. The mixture was purified by reverse column (H₂O/ACN=100/0 to 0/100) to afford (2S)-tert-butyl 2-(1-(1-(2,6-dioxopiperidin-3-yl)-3-methyl-2-oxo-2, 3-dihydro-1H-benzo[d]imidazol-5-yl)piperidin-4-yl)propanoate (7, 0.250 g, 530.56 μmol, 73% yield) as yellow solid. The stereocenters of this compound was assigned arbitrarily.

LCMS (ESI): m/z 471.1 [M+H]⁺

### Step 6: (2S)-2-(1-(1-(2,6-dioxopiperidin-3-yl)-3-methyl-2-oxo-2,3-dihydro-1H-benzo[d]imidazol-5-yl)piperidin-4-yl)propanoic acid (8)

To a solution of (2S)-tert-butyl 2-(1-(1-(2,6-dioxopiperidin-3-yl)-3-methyl-2-oxo-2, 3-dihydro-1H-benzo[d]imidazol-5-yl)piperidin-4-yl)propanoate (7, 0.13 g, 200.37 μmol) in dioxane (2 mL) was added HCl/dioxane (4 M, 2 mL). Then the mixture was stirred at 25° C. for 12 h. The reaction mixture was concentrated under reduced pressure to afford (2S)-2-(1-(1-(2,6-dioxopiperidin-3-yl)-3-methyl-2-oxo-2,3-dihydro-1H-benzo[d]imidazol-5-yl)piperidin-4-yl)propanoic acid (8, 0.15 g, 332.66 μmol, 78% yield, HCl salt) The stereo centers of this compound was assigned arbitrarily. The material was used into next step without purification.

LCMS (ESI): m/z 415.1 [M+H]⁺

### (2R)-2-(1-(1-(2,6-dioxopiperidin-3-yl)-3-methyl-2-oxo-2,3-dihydro-1H-benzo[d]imidazol-5-yl)piperidin-4-yl)propanoic acid (3)

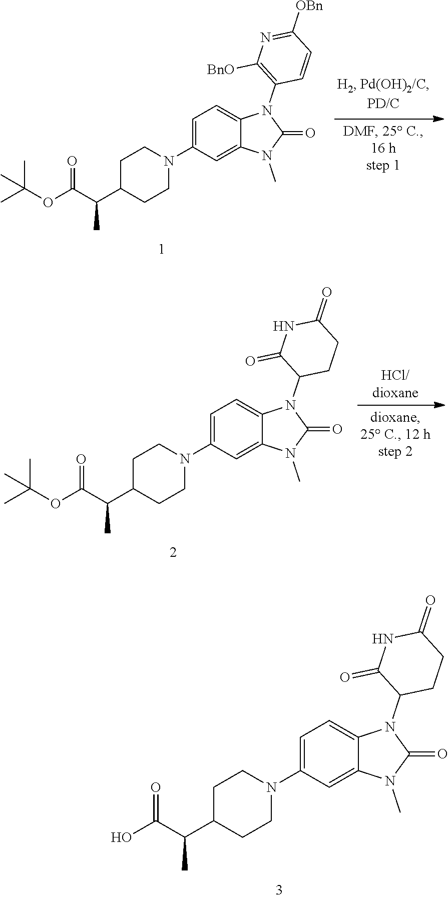 [SMILES: C[C@@H](C(=O)OC(C)(C)C)C1CCN(c2ccc3c(c2)n(C)c(=O)n3-c2ccc(OCc3ccccc3)nc2OCc2ccccc2)CC1 ; C[C@@H](C(=O)OC(C)(C)C)C1CCN(c2ccc3c(c2)n(C)c(=O)n3C2CCC(=O)NC2=O)CC1 ; C[C@@H](C(=O)O)C1CCN(c2ccc3c(c2)n(C)c(=O)n3C2CCC(=O)NC2=O)CC1]

### Step 1: (2R)-tert-butyl 2-(1-(1-(2,6-dioxopiperidin-3-yl)-3-methyl-2-oxo-2,3-dihydro-1H-benzo[d]imidazol-5-yl)piperidin-4-yl)propanoate (2)

To a solution of (R)-tert-butyl 2-(1-(1-(2,6-bis(benzyloxy) pyridin-3-yl)-3-methyl-2-oxo-2,3-dihydro-1H-benzo[d]imidazol-5-yl)piperidin-4-yl)propanoate (1, 0.260 g, 400.75 μmol) in DMF (5 mL) was added Pd/C (10% purity) and Pd(OH)₂/C (120 mg, 400.75 μmol, 10% purity). The mixture was stirred at 25° C. for 4 h under H₂ (15 Psi). The reaction mixture was filtered and concentrated under reduced pressure. T The mixture was purified by reverse column (H₂O/ACN=100/0 to 0/100) to afford (2R)-tert-butyl 2-(1-(1-(2,6-dioxopiperidin-3-yl)-3-methyl-2-oxo-2,3-dihydro-1H-benzo[d]imidazol-5-yl)piperidin-4-yl)propanoate (2, 0.17 g, 361.27 μmol, 90% yield) as white solid. The stereocenters of this compound was assigned arbitrarily.

LCMS (ESI): m/z 471.1 [M+H]⁺

### Step 2: (2R)-2-(1-(1-(2,6-dioxopiperidin-3-yl)-3-methyl-2-oxo-2,3-dihydro-1H-benzo[d]imidazol-5-yl)piperidin-4-yl)propanoic acid (3)

To a solution of (2R)-tert-butyl 2-(1-(1-(2,6-dioxopiperidin-3-yl)-3-methyl-2-oxo-2,3-dihydro-1H-benzo[d]imidazol-5-yl)piperidin-4-yl)propanoate (2, 0.2 g, 425.03 μmol) in dioxane (2 mL) was added HCl/dioxane (4 M, 2 mL) was stirred at 25° C. for 12 h. The reaction mixture was concentrated under reduced pressure to afford (2R)-2-(1-(1-(2,6-dioxopiperidin-3-yl)-3-methyl-2-oxo-2,3-dihydro-1H-benzo[d]imidazol-5-yl)piperidin-4-yl)propanoic acid (3, 0.15 g, 332.66 μmol, 78% yield, HCl salt) as white solid. The stereocenter of this compound was assigned arbitrarily. The material was used into next step without purification.

LCMS (ESI): m/z 415.1 [M+H]⁺

### 2-[3-[[1-(2,6-dibenzyloxy-3-pyridyl)-3-methyl-2-oxo-benzimidazol-5-yl]amino]pyrazol-1-yl]acetic acid (6)

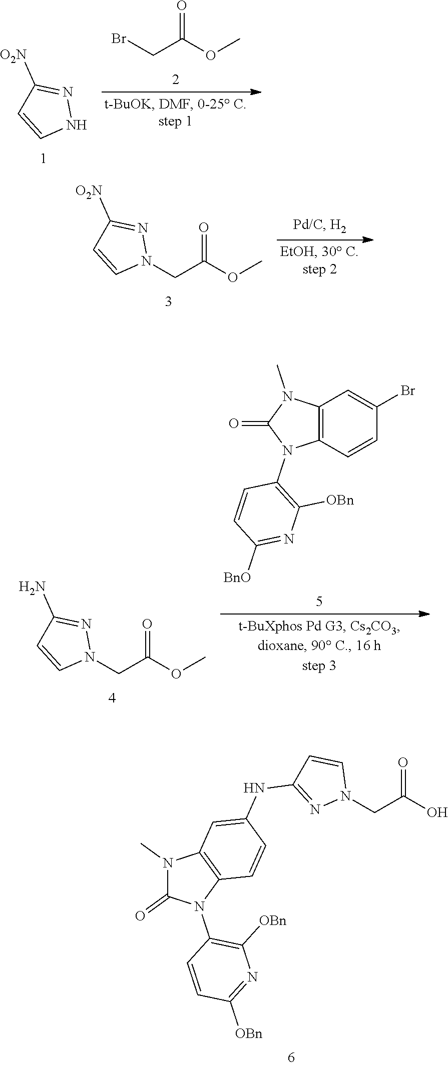

### Step 1: methyl 2-(3-nitro-1H-pyrazol-1-yl)acetate (3)

To a solution of 3-nitro-1H-pyrazole (1, 1 g, 8.84 mmol) in DMF (10 mL) was added t-BuOK (1.98 g, 17.69 mmol) at 0° C., the mixture was stirred at 0° C. for 10 min, then was added methyl 2-bromoacetate (2, 6.76 g, 44.22 mmol) at 0° C. Then the mixture was stirred at 25° C. for 12 h. The mixture was diluted with water (150 mL), extracted with EtOAc (100 mL*3) and washed with brine (30 mL*3). The organic layer was dried with anhydrous Na₂SO₄, filtered and concentrated under vacuum. The residue was purified by prep-HPLC (0.1% FA) and remove the MeCN under vacuum, then extracted with EtOAc (200 mL*3). The organic layer was dried with anhydrous Na₂SO₄, filtered and concentrated under vacuum to give methyl 2-(3-nitro-1H-pyrazol-1-yl)acetate (3, 1.6 g, 8.64 mmol, 98% yield) as yellow solid. LCMS (ESI): m/z 185.9 [M+1]⁺

### Step 2: methyl 2-(3-amino-1H-pyrazol-1-yl)acetate (4)

A mixture of methyl 2-(3-nitro-1H-pyrazol-1-yl)acetate (3, 1.6 g, 8.64 mmol) in Methanol (16 mL) was added Pd/C (320.00 mg, 263.47 μmol, 10% purity), the mixture degassed and purged with H₂ 3 time. Then the mixture was stirred at 10° C. for 16 h. The mixture was filtered, then wash with MeOH (20 mL*2). The filtrate was concentrated under vacuum. The residue was to next step without purification. Compound methyl 2-(3-amino-1H-pyrazol-1-yl)acetate (4, 1.3 g, 8.38 mmol, 97% yield) as yellow oil.

LCMS (ESI): m/z 156.1 [M+1]⁺

### Step 3: 2-[3-[[1-(2,6-dibenzyloxy-3-pyridyl)-3-methyl-2-oxo-benzimidazol-5-yl]amino]pyrazol-1-yl]acetic acid (6)

To a solution of methyl 2-(3-aminopyrazol-1-yl)acetate (4, 390.60 mg, 2.52 mmol) and 1-(2,6-bis(benzyloxy)pyridin-3-yl)-5-bromo-3-methyl-1H-benzo[d]imidazol-2 (3H)-one (5, 1 g, 1.94 mmol) in dioxane (10 mL) was added tBuXPhos-Pd-G3 (153.83 mg, 193.65 μmol), cesium carbonate (1.89 g, 5.81 mmol) under N2 atmosphere. The suspension was degassed and purged with N2 for 3 times. Then the mixture was stirred at 90° C. for 12 hrs. The reaction was washed with water (10 mL) and extracted with ethyl acetate 20 mL (10 mL*2). The aqueous phase was adjusted pH to 3 with 1N HCl aqueous. The mixture was extracted with ethyl acetate 80 mL (20 mL*4). The combined organic layers were dried over anhydrous sodium sulfate, filtered and concentrated under reduced pressure to give 2-[3-[[1-(2,6-dibenzyloxy-3-pyridyl)-3-methyl-2-oxo-benzimidazol-5-yl]amino]pyrazol-1-yl]acetic acid (6, 600 mg, 863.68 μmol, 45% yield) as brown solid.

LCMS (ESI): m/z 577.3 [M+1]⁺

### 2-((((benzyloxy)carbonyl)(4-(1-(2,6-bis(benzyloxy)pyridin-3-yl)-3-methyl-2-oxo-2,3-dihydro-1H-benzo[d]imidazol-5-yl)phenyl)amino)methyl)cyclopropanecarboxylic acid (7)

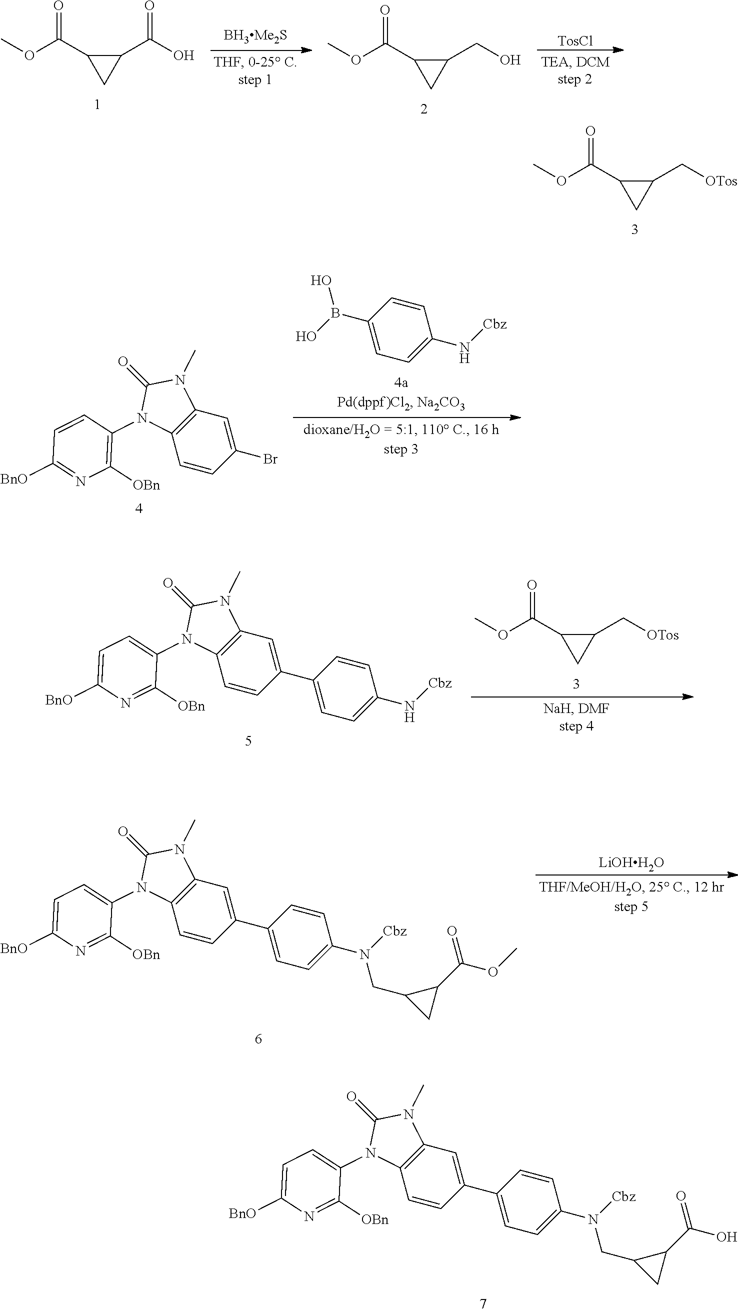

### Step 1: 2-(hydroxymethyl)cyclopropanecarboxylate (2)

To a solution of 2-(methoxycarbonyl)cyclopropanecarboxylic acid (1, 1 g, 6.94 mmol) in THF (10 mL) was added borane; methylsulfanylmethane (10 M, 1.39 mL) at −15° C. Then the mixture was stirred at 20° C. for 12 h. The mixture was quenched with MeOH (10 mL) at 0° C. and stirred at 15° C. for 2 h. Then was concentrated under vacuum to afford methyl 2-(hydroxymethyl) cyclopropanecarboxylate (2, 0.9 g, 6.92 mmol, 100% yield) as yellow oil. ¹H NMR (400 MHz, CDCl3) δ=3.79-3.74 (m, 1H), 4.00-3.74 (m, 1H), 3.72-3.68 (m, 1H), 3.70-3.67 (m, 1H), 3.64-3.44 (m, 1H), 1.80-1.55 (m, 2H), 1.28-0.86 (m, 2H).

### Step 2: methyl 2-((tosyloxy)methyl)cyclopropanecarboxylate (3)

To a solution of methyl 2-(hydroxymethyl)cyclopropanecarboxylate (2, 0.8 g, 6.15 mmol) in DCM (10 mL) was added 4-methylbenzene-1-sulfonyl chloride (1.17 g, 6.15 mmol) and TEA (1.87 g, 18.44 mmol, 2.57 mL) at 0° C. Then the mixture was stirred at 30° C. for 12 h. The mixture quenched with Sat·NaHCO₃ (10 mL), extracted with DCM (8 mL*3), the combined organic layers was washed with brine (8 mL*3), then was concentrated under vacuum. The crude product was purified by column chromatography on silica gel eluted with petroleum ether/ethyl acetate (0/1 to 1/1) to afford methyl 2-((tosyloxy)methyl)cyclopropanecarboxylate (3, 1 g, 3.52 mmol, 57% yield) as yellow oil.

¹H NMR (400 MHz, DMSO-d₆) δ=7.83-7.70 (m, 2H), 7.48 (d, J=8.3 Hz, 2H), 4.36 (dd, J=6.2, 10.6 Hz, 1H), 4.17-3.98 (m, 1H), 3.92 (dd, J=7.9, 11.1 Hz, 1H), 3.55 (d, J=15.8 Hz, 3H), 2.42 (s, 3H), 1.85 (dt, J=5.8, 8.1 Hz, 1H), 1.71-1.52 (m, 1H), 1.14 (dt, J=4.6, 8.3 Hz, 1H), 1.07-1.00 (m, 1H), 0.95-0.80 (m, 1H)

### Step 3: benzyl (4-(1-(2,6-bis(benzyloxy)pyridin-3-yl)-3-methyl-2-oxo-2,3-dihydro-1H-benzo[d]imidazol-5-yl)phenyl)carbamate (5)

To a solution of 1-(2,6-bis(benzyloxy)pyridin-3-yl)-5-bromo-3-methyl-1H-benzo[d]imidazol-2 (3H)-one (4, 1 g, 1.94 mmol), [4-(benzyloxycarbonylamino)phenyl]boronic acid (4a, 787.42 mg, 2.90 mmol) and disodium; carbonate (615.76 mg, 5.81 mmol, 243.38 μL) in Dioxane (10 mL) and Water (2 mL) was added cyclopentyl(diphenyl)phosphane; dichloropalladium; iron (283.40 mg, 387.31 μmol). Then the mixture was stirred at 110° C. for 5 h under N₂. The mixture was filtered and the filter was concentrated under vacuum. The crude product was purified by column chromatography on silica gel eluted with petroleum ether/ethyl acetate (0/1 to 1/1) Compound benzyl (4-(1-(2,6-bis(benzyloxy)pyridin-3-yl)-3-methyl-2-oxo-2,3-dihydro-1H-benzo[d]imidazol-5-yl)phenyl)carbamate (5, 1.1 g, 1.66 mmol, 86% yield) as yellow solid.

LCMS (ESI): m/z 662.9 [M+H]⁺

### Step 4: methyl 2-((((benzyloxy)carbonyl)(4-(1-(2,6-bis(benzyloxy)pyridin-3-yl)-3-methyl-2-oxo-2,3-dihydro-1H-benzo[d]imidazol-5-yl)phenyl)amino)methyl)cyclopropanecarboxylate (6)

To a solution of benzyl (4-(1-(2,6-bis(benzyloxy)pyridin-3-yl)-3-methyl-2-oxo-2,3-dihydro-1H-benzo[d]imidazol-5-yl)phenyl)carbamate (5, 900 mg, 1.36 mmol) in DMF (5 mL) was added NaH (108.63 mg, 2.72 mmol, 60% purity) at 0° C. Then the mixture was stirred at 0° C. for 0.5 h. Then a solution of methyl 2-((tosyloxy)methyl)cyclopropanecarboxylate (3, 772.24 mg, 2.72 mmol) in DMF (4 mL) was added. Then the mixture was stirred at 25° C. for 12 h. The reaction mixture was quenched by water (20 mL), extracted with EtOAc (30 mL*3) and concentrated to get a residue. The residue was purified by reversed phase HPLC (FA) and then lyophilization. The methyl 2-((((benzyloxy)carbonyl)(4-(1-(2,6-bis(benzyloxy)pyridin-3-yl)-3-methyl-2-oxo-2,3-dihydro-1H-benzo[d]imidazol-5-yl)phenyl)amino)methyl)cyclopropanecarboxylate (6, 460 mg, 587.72 μmol, 43% yield) as yellow solid.

LCMS (ESI): m/z 775.2 [M+H]⁺

### Step 5: 2-((((benzyloxy)carbonyl)(4-(1-(2,6-bis(benzyloxy)pyridin-3-yl)-3-methyl-2-oxo-2,3-dihydro-1H-benzo[d]imidazol-5-yl)phenyl)amino)methyl)cyclopropanecarboxylic acid (7)

To a solution of To a solution of methyl 2-[[N-benzyloxycarbonyl-4-[1-(2,6-dibenzyloxy-3-pyridyl)-3-methyl-2-oxo-benzimidazol-5-yl]anilino]methyl]cyclopropanecarboxylate (6, 460 mg, 593.66 μmol) in THF (2 mL) and MeOH (2 mL) was added a solution of LiOH·H₂O (74.74 mg, 1.78 mmol) in Water (2 mL). The mixture was stirred at 25° C. for 2 h. The reaction was quenched by 1N aq·HCl and adjust pH to 6˜7 then concentrated to get a residue. The residue was purified by reversed phase HPLC (FA) and then lyophilization. The 2-((((benzyloxy)carbonyl)(4-(1-(2,6-bis(benzyloxy)pyridin-3-yl)-3-methyl-2-oxo-2,3-dihydro-1H-benzo[d]imidazol-5-yl)phenyl)amino)methyl)cyclopropanecarboxylic acid (7, 263 mg, 335.30 μmol, 56% yield) as yellow solid.

LCMS (ESI): m/z 761.2 [M+H]⁺

### 2-((1R,3r,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetic acid (8)

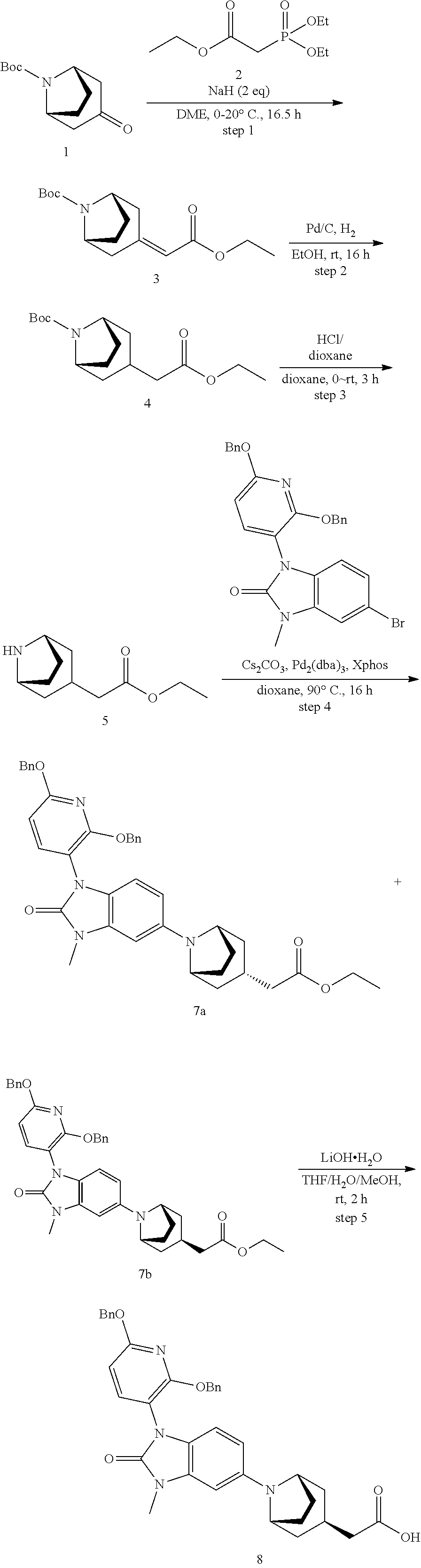

Note: Configurations are arbitrarily assigned.

### Step 1: tert-butyl 3-(2-ethoxy-2-oxoethylidene)-8-azabicyclo[3.2.1]octane-8-carboxylate (3)

To a solution of ethyl 2-(diethoxyphosphoryl)acetate (2, 9.95 g, 44.39 mmol, 8.81 mL, 2 eq in DME (50 mL) was added NaH (1.70 g, 44.39 mmol, 60% purity, 2 eq at 0° C. and stirred at 20° C. for 0.5 h. Then tert-butyl 3-oxo-8-azabicyclo[3.2.1]octane-8-carboxylate (1, 5 g, 22.19 mmol, 1 eq) was added to the mixture at 0° C. The reaction mixture was stirred at 20° C. for 16 h. The reaction mixture was quenched by addition of saturated NH₄Cl (100 mL) at 0° C. and extracted with ethyl acetate (60 mL×3). The combined organic layers were washed with brine (100 mL), dried over Na₂SO₄, filtered and concentrated under reduced pressure to give residue. The residue was purified by flash silica gel chromatography (60 mL/min, Eluent of 0-50% ethyl acetate/petroleum ether gradient, Column: ISCO; 40 g SepaFlash Silica Flash Column), to afford tert-butyl 3-(2-ethoxy-2-oxoethylidene)-8-azabicyclo[3.2.1]octane-8-carboxylate (3, 6.5 g, 21.35 mmol, 96% yield) as yellow oil.

LCMS (ESI): m/z 240.2 [M−tBu+H]⁺

### Step 2: tert-butyl 3-(2-ethoxy-2-oxoethyl)-8-azabicyclo[3.2.1]octane-8-carboxylate (4)

To a solution of tert-butyl 3-(2-ethoxy-2-oxoethylidene)-8-azabicyclo[3.2.1]octane-8-carboxylate (3, 6.5 g, 22.01 mmol, 1 eq) in Ethanol (100 mL) was added Pd/C (700 mg, 10% purity) under N₂ atmosphere. The suspension was degassed and purged with H₂ 3 times. The mixture was stirred under H₂ (15 Psi) at 20° C. for 16 h. The reaction mixture was filtered. The filtrate was concentrated under reduced pressure to afford tert-butyl 3-(2-ethoxy-2-oxoethyl)-8-azabicyclo[3.2.1]octane-8-carboxylate (4, 5.8 g, 16.77 mmol, 76% yield) as white oil. The crude product was used in the next step without further purification.

LCMS (ESI): m/z 242.1 [M−tBu+H]⁺

### Step 3: ethyl 2-(8-azabicyclo[3.2.1]octan-3-yl)acetate (5)

To a solution of tert-butyl 3-(2-ethoxy-2-oxoethyl)-8-azabicyclo[3.2.1]octane-8-carboxylate (4, 3.8 g, 12.78 mmol) in dioxane (3 mL) was added HCl/dioxane (4 M, 38.00 mL) at 0° C. The mixture was stirred at 20° C. for 3 h. The reaction mixture was concentrated under reduced pressure to afford ethyl 2-(8-azabicyclo[3.2.1]octan-3-yl)acetate (5, 2.5 g, 10.70 mmol, 84% yield, HCl salt) as a white solid. The crude product was used in the next step without further purification. ¹H NMR (400 MHz, DMSO-d₆) δ=4.05 (dq, J=4.4, 7.2 Hz, 2H), 3.88 (s, 2H), 3.39 (s, 2H), 2.53 (d, J=7.6 Hz, 1H), 2.30-2.11 (m, 3H), 2.03-1.92 (m, 2H), 1.89-1.76 (m, 2H), 1.67-1.60 (m, 2H), 1.59-1.44 (m, 1H), 1.17 (t, J=1.6, 7.2 Hz, 3H).

### Step 4: ethyl 2-((1R,3r,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetate (7b)

A mixture of ethyl 2-(8-azabicyclo[3.2.1]octan-3-yl)acetate (5, 764.06 mg, 3.87 mmol, HCl salt) and 1-(2,6-bis(benzyloxy)pyridin-3-yl)-5-bromo-3-methyl-1H-benzo[d]imidazol-2 (3H)-one (6, 1 g, 1.94 mmol) in dioxane (15 mL) were added Cs₂CO₃ (1.89 g, 5.81 mmol), Pd₂(dba)₃ (88.67 mg, 96.83 μmol) and Xphos (92.32 mg, 193.65 μmol). The mixture was degassed and purged with N₂ 3 times, and then the mixture was stirred at 90° C. for 16 h under N₂ atmosphere. The reaction mixture was filtered and the filtrate was concentrated under reduced pressure to give residue. The residue was purified by flash silica gel chromatography (60 mL/min, Eluent of 0-50% ethyl acetate/petroleum ether gradient, Column: ISCO; 10 g SepaFlash SilicaFlash Column) to afford 680 mg crude product. The crude product was purified by prep-HPLC (flow: 25 mL/min; gradient: from 54-74% MeCN-water (0.1% TFA) over 7 min; column: 3_Phenomenex Luna C18 75×30 mm×3 um) to afford ethyl 2-((1R,3s,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetate (7a, 210 mg, 704.07 μmol, 16.7% yield) as a pink solid and ethyl 2-((1R,3r,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetate (7b, 120 mg, 413.91 μmol, 9% yield) as a pink solid.

LCMS (ESI): m/z 633.5 [M+H]⁺

### Step 5: 2-((1R,3r,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetic acid (8)

To a solution of ethyl 2-((1R,3r,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetate (7b, 120.00 mg, 189.6 μmol, 1 eq) in THF (3 mL), H₂O (1 mL) and MeOH (2 mL) was added LiOH H₂O (39.79 mg, 948.25 μmol). The mixture was stirred at 10° C. for 1 h. The reaction mixture was diluted with water (5 mL) and washed with ethyl acetate (5 mL). The organic layers were discarded and the aqueous phase was adjusted pH to 3 with 1 N aqueous HCl. The mixture was extracted with ethyl acetate (10 mL×3). The combined organic layers were washed with brine (10 mL), dried over Na₂SO₄, filtered and concentrated under reduced pressure to afford 2-((1R,3r,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetic acid (8, 100 mg, 128.99 μmol, 68% yield) as a white solid. The crude product was used in the next step without further purification.

LCMS (ESI): m/z 605.4 [M+H]⁺

### 2-((1R,3s,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetic acid (2)

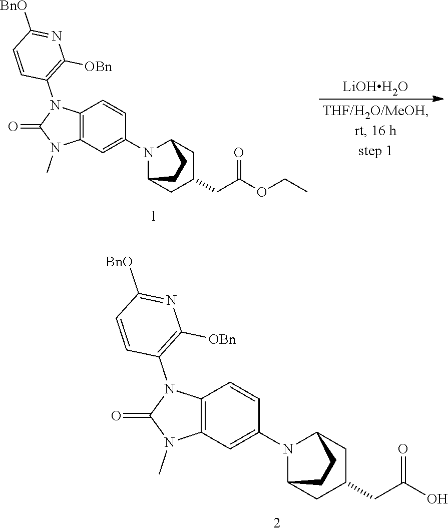

Note: Configurations are arbitrarily assigned.

### Step 1: 2-((1R,3s,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetic acid (2)

To a solution of ethyl 2-((1R,3s,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetate (1, 210 mg, 331.89 μmol) in THF (3 mL), H₂O (2 mL) and MeOH (2 mL) was added LiOH·H₂O (69.64 mg, 1.66 mmol). The mixture was stirred at 10° C. for 1 h. The reaction mixture was diluted with water (5 mL) and extracted with ethyl acetate (5 mL), Then the organic layers was discarded and the aqueous phase was acidified to pH=3 with 1 N aqueous HCl, the mixture was extracted with ethyl acetate (10 mL×3). The combined organic layers were washed with brine (10 mL), dried over Na₂SO₄, filtered and concentrated under reduced pressure to afford 2-((1R,3s,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetic acid (2, 190 mg, 273.36 μmol, 82% yield) as a white solid.

LCMS (ESI): m/z 605.4 [M+H]⁺

### 2-((3S,4S)-4-(1-(2,6-dioxopiperidin-3-yl)-3-ethyl-2-oxo-2,3-dihydro-1H-benzo[d]imidazol-5-yl)-3-hydroxypiperidin-1-yl)acetic acid (11a)

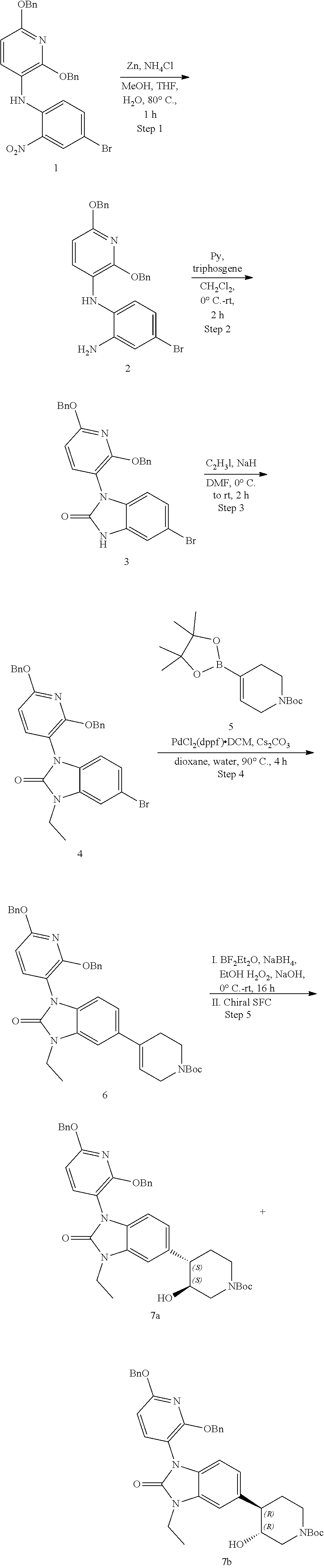

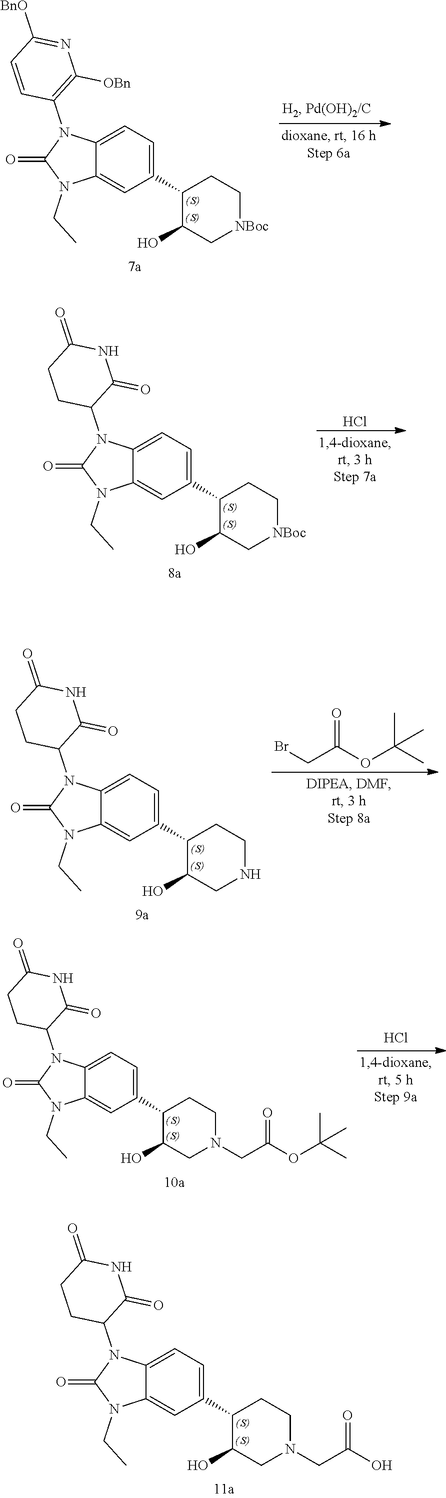

Note: Configurations are arbitrarily assigned.

### Step 1: 4-bromo-N1-(2,6-dibenzyloxy-3-pyridyl)benzene-1,2-diamine (2)

Into a 500 mL three-neck round-bottom flask containing a well-stirred solution of 2,6-dibenzyloxy-N-(4-bromo-2-nitro-phenyl)pyridin-3-amine (1, 5 g, 9.78 mmol) in methanol (50 mL), THF (40 mL) and water (10 mL) were added zinc powder (325 mesh High Grade Material, 3.20 g, 48.88 mmol) and ammonium chloride (2.61 g, 48.88 mmol, 1.71 mL) at 0° C. The reaction mixture was stirred for 1 h at 80° C. Afterwards the reaction mixture was passed through Celite and washed with EtOAc (300 mL). Ethyl acetate layer was washed with water (2×100 mL), dried over anhydrous Na₂SO₄, filtered and concentrated under reduced pressure to afford 4-bromo-N₁-(2,6-dibenzyloxy-3-pyridyl)benzene-1,2-diamine (2, 4.5 g, 9.12 mmol, 93% yield) as brown solid.

LCMS (ES+): m/z 477.0 [M+H]⁺

### Step 2: 6-bromo-3-(2,6-dibenzyloxy-3-pyridyl)-1H-benzimidazol-2-one (3)

Into a 1 litre three-neck round-bottom flask containing a well-stirred solution of 4-bromo-N₁-(2,6-dibenzyloxy-3-pyridyl)benzene-1,2-diamine (2, 8.5 g, 17.22 mmol) in anhydrous DCM (150 mL) was added triphosgene (10.22 g, 34.44 mmol) at 0° C. and stirred for 5 min. Then Pyridine (6.81 g, 86.09 mmol, 6.96 mL) in anhydrous DCM (30 mL) was added at 0° C. The resulting mixture was stirred at room temperature for 2 h. Afterwards, DCM (500 mL) was added to the reaction mixture and the organic layer was washed with water (2×200 mL). The organic layer was dried over sodium sulfate, concentrated and subjected to purification by a silica gel flash column chromatography (230-400 mesh silica gel; 30% EtOAc in pet ether) to afford 6-bromo-3-(2,6-dibenzyloxy-3-pyridyl)-1H-benzimidazol-2-one (3, 8.0 g, 14.89 mmol, 86% yield) as an off-white solid.

LCMS (ES+): m/z 502.0 [M+H]⁺

### Step 3: 5-bromo-1-(2,6-dibenzyloxy-3-pyridyl)-3-ethyl-benzimidazol-2-one (4)

Into a 500 mL three-neck round-bottom flask containing a well-stirred solution of 6-bromo-3-(2,6-dibenzyloxy-3-pyridyl)-1H-benzimidazol-2-one (3, 7.5 g, 13.88 mmol) in dry DMF (60 mL) was added sodium hydride (60% dispersion in mineral oil, 798.01 mg, 20.83 mmol) at 0° C. and the resulting suspension was stirred at rt for 30 min. Afterwards, iodoethane (4.33 g, 27.77 mmol, 2.23 mL) was added and the stirring was continued at room temperature for 2 h. The reaction mixture was quenched with ice cold water (100 mL) and the solution was extracted with ethyl acetate (2×300 mL). The combined organic layers were dried over Na₂SO₄ and concentrated under reduced pressure to get the crude material that was purified by silica gel flash column chromatography (230-400 sillica gel, 40% EtOAc in pet ether) to afford 5-bromo-1-(2,6-dibenzyloxy-3-pyridyl)-3-ethyl-benzimidazol-2-one (4, 7.5 g, 13.18 mmol, 95% yield) as brown sticky solid.

LCMS (ES+): m/z 532.0 [M+H]⁺

### Step 4: tert-butyl 4-[1-(2,6-dibenzyloxy-3-pyridyl)-3-ethyl-2-oxo-benzimidazol-5-yl]-3,6-dihydro-2H-pyridine-1-carboxylate (6)

Into a 250 mL sealed-tube containing a well-stirred solution of 5-bromo-1-(2,6-dibenzyloxy-3-pyridyl)-3-ethyl-benzimidazol-2-one (4, 3.0 g, 5.26 mmol) and tert-butyl 4-(4,4,5,5-tetramethyl-1,3,2-dioxaborolan-2-yl)-3,6-dihydro-2H-pyridine-1-carboxylate (5, 1.95 g, 6.31 mmol) in 1,4-dioxane (40 mL) and water (8 mL) was added caesium carbonate (3.43 g, 10.52 mmol) at room temperature. The reaction mixture was purged with nitrogen gas for 5 min. Then Pd(dppf)Cl₂ CH₂Cl₂ (644.33 mg, 789.01 μmol) was added and reaction mixture was heated at 90° C. for 4 h. The reaction mixture was filtered through Celite and washed thoroughly with EtOAc (400 mL). The filtrate was washed with water (100 mL), dried over Na₂SO₄, filtered and concentrated under reduced pressure. The crude material was purified by silica gel flash column chromatography (230-400 silica gel, 30% EtOAc in pet ether) to afford tert-butyl 4-[1-(2,6-dibenzyloxy-3-pyridyl)-3-ethyl-2-oxo-benzimidazol-5-yl]-3,6-dihydro-2H-pyridine-1-carboxylate (6, 3.9 g, 5.19 mmol, 99% yield) as brown sticky solid.

LCMS (ES+): m/z 633.2 [M+H]⁺

### Step 5: tert-butyl (3S,4S)-4-[1-(2,6-dibenzyloxy-3-pyridyl)-3-ethyl-2-oxo-benzimidazol-5-yl]-3-hydroxy-piperidine-1-carboxylate (7a)

### and

### tert-butyl (3R,4R)-4-[1-(2,6-dibenzyloxy-3-pyridyl)-3-ethyl-2-oxo-benzimidazol-5-yl]-3-hydroxy-piperidine-1-carboxylate (7b)

Into a 500 mL three-neck round-bottom flask containing a well-stirred solution of sodium borohydride (929.15 mg, 24.56 mmol, 868.37 μL) in anhydrous THF (20 mL) was added boron trifluoride diethyl etherate (3.49 g, 24.56 mmol, 3.08 mL) at 0° C. and the resulting mixture was stirred for 2 h at room temperature. Then tert-butyl 4-[1-(2,6-dibenzyloxy-3-pyridyl)-3-ethyl-2-oxo-benzimidazol-5-yl]-3,6-dihydro-2H-pyridine-1-carboxylate (6, 3.7 g, 4.91 mmol) in anhydrous THF (50 mL) was added at 0° C. The contents were stirred for 3 h at room temperature. The reaction was quenched with water (10 mL), ethanol (25 mL), 10% NaOH solution (25 mL) and H₂O₂ (27.50 g, 242.54 mmol, 25 mL, 30% purity) at 0° C. The reaction mixture was stirred at ambient temperature for 11 h. Afterwards, water (100 mL) was added to the reaction mixture and extracted with EtOAc (2×300 mL). The combined organic layers were washed with brine solution (50 mL), dried over Na₂SO₄, filtered and concentrated under reduced pressure. The crude material was purified by silica gel flash column chromatography using (230-400 silica gel; 50% EtOAc in pet ether) to afford tert-butyl-4-[1-(2,6-dibenzyloxy-3-pyridyl)-3-ethyl-2-oxo-benzimidazol-5-yl]-3-hydroxy-piperidine-1-carboxylate (7).

### Chiral Separation:

The enantiomers were separated by chiral SFC: Method details: Column Name: YMC Amylose-SA; Co-Solvent: 30% and Co-Solvent Name: IPA; Outlet Pressure: 100 bar; Temperature: 35° C. After the separation first eluted isomer tert-butyl (3S,4S)-4-[1-(2,6-dibenzyloxy-3-pyridyl)-3-ethyl-2-oxo-benzimidazol-5-yl]-3-hydroxy-piperidine-1-carboxylate (7a, 900 mg, 1.35 mmol, 27% yield) (RT 3.66, optical purity 97.07%), arbitrarily assigned as (3S,4S)-isomer was isolated as white solid.

LCMS (ES+): m/z 651.2 [M+H]⁺

and
the second eluted isomer tert-butyl (3R,4R)-4-[1-(2,6-dibenzyloxy-3-pyridyl)-3-ethyl-2-oxo-benzimidazol-5-yl]-3-hydroxy-piperidine-1-carboxylate (7b, 800 mg, 1.23 mmol, 25% yield) (RT 4.66, optical purity 99.95%), arbitrarily assigned as (3R,4R)-isomer was isolated as an off-white solid.

LCMS (ES+): m/z 651.2 [M+H]⁺

### Step 6a: tert-butyl (3S,4S)-4-(1-(2,6-dioxopiperidin-3-yl)-3-ethyl-2-oxo-2,3-dihydro-1H-benzo[d]imidazol-5-yl)-3-hydroxypiperidine-1-carboxylate (8a)

Into a 100 mL single-neck round-bottom flask containing a well-stirred solution of tert-butyl (3S,4S)-4-[1-(2,6-dibenzyloxy-3-pyridyl)-3-ethyl-2-oxo-benzimidazol-5-yl]-3-hydroxy-piperidine-1-carboxylate (7a, 400 mg, 596.22 μmol) in anhydrous 1,4-dioxane (15 mL) was added palladium hydroxide on carbon (20 wt. % 50% water, 500 mg, 3.56 mmol) at room temperature. The mixture was stirred at room temperature for 16 h under hydrogen gas bladder pressure. The reaction mixture was passed through Celite and washed with 1,4-dioxane (50 mL). The filtrate was concentrated under reduced pressure to afford tert-butyl (3S,4S)-4-[1-(2,6-dioxo-3-piperidyl)-3-ethyl-2-oxo-benzimidazol-5-yl]-3-hydroxy-piperidine-1-carboxylate (8a, 280 mg, 588.40 μmol, 99% yield) as an off-white solid.

LCMS (ES+): m/z 417.2 [M−tBu+H]⁺

### Step 7a: 3-(3-ethyl-5-((3S,4S)-3-hydroxypiperidin-4-yl)-2-oxo-2,3-dihydro-1H-benzo[d]imidazol-1-yl)piperidine-2,6-dione (9a)

Into a 25 mL single-neck round-bottom flask containing a well-stirred solution of tert-butyl (3R,4R)-4-[1-(2,6-dioxo-3-piperidyl)-3-ethyl-2-oxo-benzimidazol-5-yl]-3-hydroxy-piperidine-1-carboxylate (8a, 280 mg, 586.63 μmol) in dry 1,4-dioxane (2 mL) was added HCl (11.73 mmol, 3.0 mL) at 0° C. The resultant reaction mixture was stirred for 3 h at room temperature. The solvent was evaporated and the residue was washed with MTBE (25 mL) to afford 3-[3-ethyl-5-[(3S,4S)-3-hydroxy-4-piperidyl]-2-oxo-benzimidazol-1-yl]piperidine-2,6-dione (9a, 200 mg, 473.88 μmol, 81% yield, HCl salt) as white solid.

LCMS (ES+): m/z 373.2 [M+H]⁺

### Step 8a: tert-butyl 2-((3S,4S)-4-(1-(2,6-dioxopiperidin-3-yl)-3-ethyl-2-oxo-2,3-dihydro-1H-benzo[d]imidazol-5-yl)-3-hydroxypiperidin-1-yl)acetate (10a)

Into a 100 mL single-neck round-bottom flask containing a well-stirred solution of 3-[3-ethyl-5-[(3S,4S)-3-hydroxy-4-piperidyl]-2-oxo-benzimidazol-1-yl]piperidine-2,6-dione (9a, 220 mg, 516.54 μmol, HCl salt) in anhydrous DMF (3 mL) at 0° C. under nitrogen atmosphere, were added tert-butyl bromoacetate (100.75 mg, 516.54 μmol, 75.75 μL) followed by DIPEA (166.89 mg, 1.29 mmol, 224.92 uL). The resulting reaction mixture was stirred at 0° C. for 3 h. The mixture was concentrated under reduced pressure to get a residue that was diluted with water (5 mL) and extracted with DCM (2×100 mL). The combined organic layers were dried over Na₂SO₄, filtered, concentrated under reduced pressure to afford tert-butyl 2-[(3S,4S)-4-[1-(2,6-dioxo-3-piperidyl)-3-ethyl-2-oxo-benzimidazol-5-yl]-3-hydroxy-1-piperidyl]acetate (10a, 250 mg, 498.40 μmol, 96% yield) as brown gummy solid.

LCMS (ES+): m/z 487.2 [M+H]⁺

### Step 9a: 2-((3S,4S)-4-(1-(2,6-dioxopiperidin-3-yl)-3-ethyl-2-oxo-2,3-dihydro-1H-benzo[d]imidazol-5-yl)-3-hydroxypiperidin-1-yl)acetic acid (11a)

Into a 50 mL single-neck round-bottom flask containing a well-stirred solution of tert-butyl 2-[(3S,4S)-4-[1-(2,6-dioxo-3-piperidyl)-3-ethyl-2-oxo-benzimidazol-5-yl]-3-hydroxy-1-piperidyl]acetate (10a, 250 mg, 498.40 μmol) in dry 1,4-dioxane (2 mL) was added HCl (4.0 M in 1,4-dioxane, 9.97 mmol, 2.5 mL) at 0° C. The resultant reaction mixture was stirred for 5 h at room temperature. The reaction mixture was concentrated and the resultant crude material was washed with MTBE (5 mL) to afford 2-[(3S,4S)-4-[1-(2,6-dioxo-3-piperidyl)-3-ethyl-2-oxo-benzimidazol-5-yl]-3-hydroxy-1-piperidyl]acetic acid (11a, 200 mg, 384.61 μmol, 77% yield, HCl salt) as brown gummy solid.

LCMS (ES+): m/z 431.2 [M+H]⁺

### 2-((3R,4R)-4-(1-(2,6-dioxopiperidin-3-yl)-3-ethyl-2-oxo-2,3-dihydro-1H-benzo[d]imidazol-5-yl)-3-hydroxypiperidin-1-yl)acetic acid (11b)

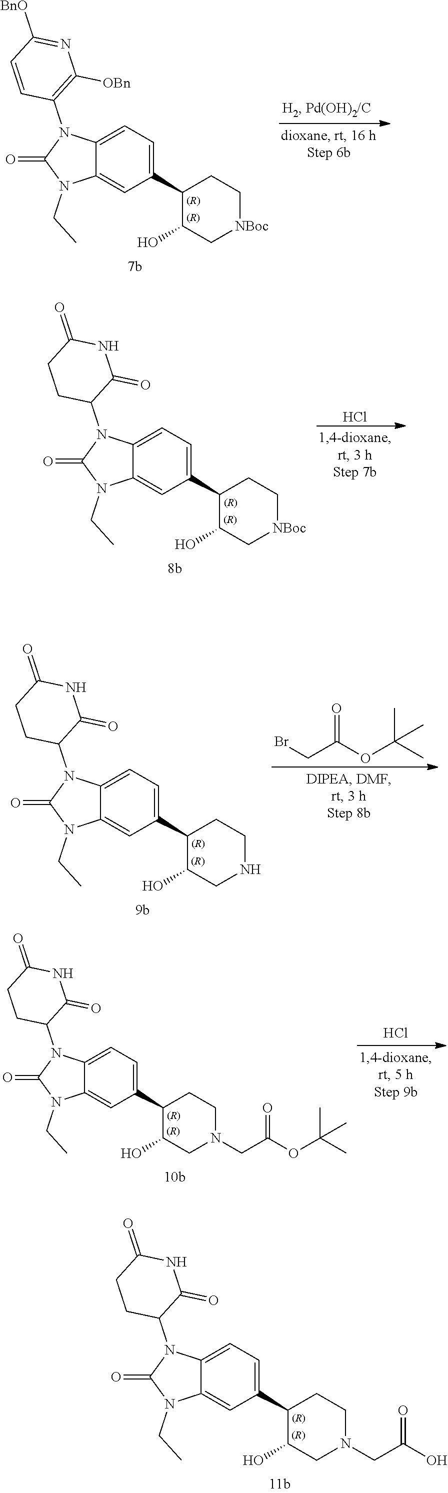

Note: Configurations are arbitrarily assigned.

### Step 6b: tert-butyl (3R,4R)-4-[1-(2,6-dioxo-3-piperidyl)-3-ethyl-2-oxo-benzimidazol-5-yl]-3-hydroxy-piperidine-1-carboxylate (8b)

Into a 100 mL single-neck round-bottom flask containing a well-stirred solution of tert-butyl (3R,4R)-4-[1-(2,6-dibenzyloxy-3-pyridyl)-3-ethyl-2-oxo-benzimidazol-5-yl]-3-hydroxy-piperidine-1-carboxylate (7b, 300 mg, 460.08 μmol) in anhydrous 1,4-dioxane (15 mL) was added palladium hydroxide on carbon (20 wt. % 50% water) (400.00 mg, 2.85 mmol) at room temperature. The mixture was stirred at room temperature for 16 h under hydrogen gas bladder pressure. The reaction mixture was passed through Celite and washed with 1,4-dioxane (50 mL). The filtrate was concentrated under reduced pressure to afford tert-butyl (3R,4R)-4-[1-(2,6-dioxo-3-piperidyl)-3-ethyl-2-oxo-benzimidazol-5-yl]-3-hydroxy-piperidine-1-carboxylate (8b, 210 mg, 443.52 μmol, 96% yield) as an off-white solid.

LCMS (ES+): m/z 417.2 [M−tBu+H]⁺

### Step 7b: 3-(3-ethyl-5-((3R,4R)-3-hydroxypiperidin-4-yl)-2-oxo-2,3-dihydro-1H-benzo[d]imidazol-1-yl)piperidine-2,6-dione (9b)

Into a 50 mL single-neck round-bottom flask containing a well-stirred solution of tert-butyl (3R,4R)-4-[1-(2,6-dioxo-3-piperidyl)-3-ethyl-2-oxo-benzimidazol-5-yl]-3-hydroxy-piperidine-1-carboxylate (8b, 210 mg, 439.97 μmol) in dry 1,4-dioxane (1 mL) was added HCl (4M in 1,4-dioxane, 8.80 mmol, 2.2 mL) at 0° C. The resultant reaction mixture was stirred for 3 h at room temperature. The solvent was evaporated and the residue was washed with MTBE (5 mL) to afford 3-[3-ethyl-5-[(3R,4R)-3-hydroxy-4-piperidyl]-2-oxo-benzimidazol-1-yl]piperidine-2,6-dione (9b, 180 mg, 430.10 μmol, 98% yield, HCl salt) as white solid.

LCMS (ES+): m/z 373.2 [M+H]⁺

### Step 8b: tert-butyl 2-((3R,4R)-4-(1-(2,6-dioxopiperidin-3-yl)-3-ethyl-2-oxo-2,3-dihydro-1H-benzo[d]imidazol-5-yl)-3-hydroxypiperidin-1-yl)acetate (10b)

Into a 100 mL single-neck round-bottom flask containing a well-stirred solution of 3-[3-ethyl-5-[(3R,4R)-3-hydroxy-4-piperidyl]-2-oxo-benzimidazol-1-yl]piperidine-2,6-dione (9b, 160 mg, 383.49 μmol, HCl salt) in anhydrous DMF (2 mL) at 0° C. under nitrogen atmosphere, were added tert-butyl bromoacetate (74.80 mg, 383.49 μmol, 56.24 μL) followed by DIPEA (99.12 mg, 766.98 μmol, 133.59 uL). The resulting reaction mixture was stirred at 0° C. for 3 h. The mixture was concentrated under reduced pressure to get a residue that was diluted with water (5 mL) and extracted with DCM (2×100 mL). The combined organic layers were dried over Na₂SO₄, filtered, concentrated under reduced pressure to afford tert-butyl 2-[(3R,4R)-4-[1-(2,6-dioxo-3-piperidyl)-3-ethyl-2-oxo-benzimidazol-5-yl]-3-hydroxy-1-piperidyl]acetate (10b, 190 mg, 382.69 μmol, 100% yield) as brown gummy solid.

LCMS (ES+): m/z 487.2 [M+H]⁺

### Step 9b: 2-((3R,4R)-4-(1-(2,6-dioxopiperidin-3-yl)-3-ethyl-2-oxo-2,3-dihydro-1H-benzo[d]imidazol-5-yl)-3-hydroxypiperidin-1-yl)acetic acid (11b)

Into a 50 mL single-neck round-bottom flask containing a well-stirred solution of tert-butyl 2-[(3R,4R)-4-[1-(2,6-dioxo-3-piperidyl)-3-ethyl-2-oxo-benzimidazol-5-yl]-3-hydroxy-1-piperidyl]acetate (10b, 190 mg, 382.69 μmol) in dry 1,4-dioxane (2 mL) was added HCl (4.0 M in 1,4-dioxane, 1.52 g, 7.65 mmol, 1.9 mL) at 0° C. The resultant reaction mixture was stirred for 5 h at room temperature. The reaction mixture was concentrated and the resultant crude material was washed with MTBE (5 mL) to afford 2-[(3R,4R)-4-[1-(2,6-dioxo-3-piperidyl)-3-ethyl-2-oxo-benzimidazol-5-yl]-3-hydroxy-1-piperidyl]acetic acid (11b, 180 mg, 376.26 μmol, 98% yield, HCl salt) as brown gummy solid.

LCMS (ES+): m/z 431.2 [M+H]⁺

### 2-(4-(1-(2,6-dioxopiperidin-3-yl)-3-methyl-2-oxo-2,3-dihydro-1H-benzo[d]imidazol-5-yl)-3-oxopiperazin-1-yl)acetic acid (7)

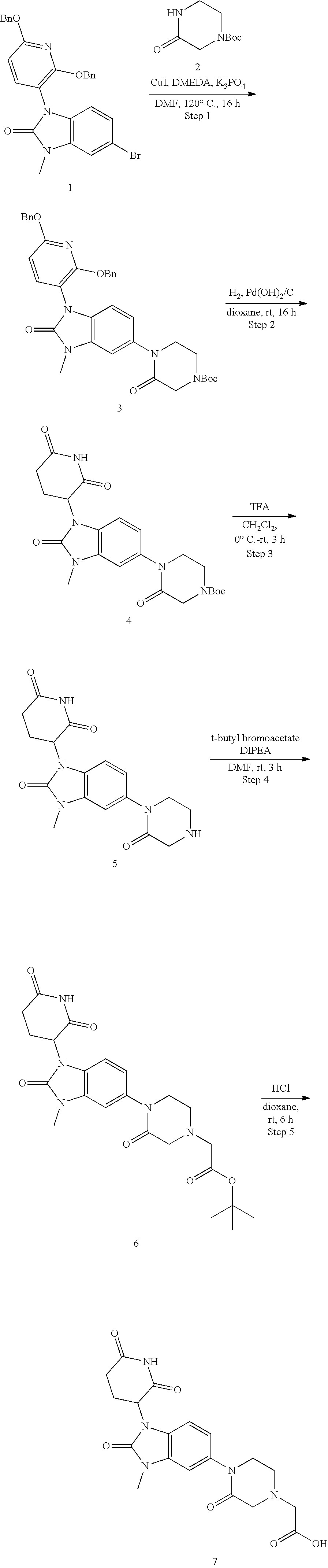

### Step 1: tert-butyl 4-(1-(2,6-bis(benzyloxy)pyridin-3-yl)-3-methyl-2-oxo-2,3-dihydro-1H-benzo[d]imidazol-5-yl)-3-oxopiperazine-1-carboxylate (3)

Into a 250 mL sealed-tube containing a well-stirred solution of 5-bromo-1-(2,6-dibenzyloxy-3-pyridyl)-3-methyl-benzimidazol-2-one (1, 2.18 g, 3.80 mmol), tert-butyl 3-oxopiperazine-1-carboxylate (2, 1.14 g, 5.69 mmol) in dry DMF (50 mL) was added potassium phosphate tribasic anhydrous (1.61 g, 7.59 mmol) and DMEDA (501.88 mg, 5.69 mmol, 613.54 uL). The reaction mixture was degassed by bubbling nitrogen for 5 min. Subsequently, copper(I) iodide (542.16 mg, 2.85 mmol, 96.47 μL) was added and the reaction mixture was heated at 120° C. for 16 h. The reaction mixture was filtered through Celite and washed with 10% MeOH in ethyl acetate (100 mL). The filtrate was concentrated under reduced pressure to get the crude material that was purified by silica gel flash column chromatography (230-400 mesh silica gel; 70% EtOAc in pet ether) to afford tert-butyl 4-[1-(2,6-dibenzyloxy-3-pyridyl)-3-methyl-2-oxo-benzimidazol-5-yl]-3-oxo-piperazine-1-carboxylate (3, 1.7 g, 2.35 mmol, 62% yield) as an off-white solid.

LCMS (ES+): m/z 636.2 [M+H]⁺

### Step 2: tert-butyl 4-(1-(2,6-dioxopiperidin-3-yl)-3-methyl-2-oxo-2,3-dihydro-1H-benzo[d]imidazol-5-yl)-3-oxopiperazine-1-carboxylate (4)

Into a 100 mL single-neck round-bottom flask containing a well-stirred solution of tert-butyl 4-[1-(2,6-dibenzyloxy-3-pyridyl)-3-methyl-2-oxo-benzimidazol-5-yl]-3-oxo-piperazine-1-carboxylate (3, 1.7 g, 2.33 mmol) in anhydrous 1,4-dioxane (30 mL) was added palladium hydroxide on carbon (20 wt. % 50% water, 1.7 g, 12.10 mmol) at room temperature and the resulting mixture was hydrogenated with a hydrogen gas bladder for 16 h. The reaction mixture was passed through Celite and washed with 1,4-dioxane (50 mL). The filtrate was concentrated under reduced pressure to afford tert-butyl 4-[1-(2,6-dioxo-3-piperidyl)-3-methyl-2-oxo-benzimidazol-5-yl]-3-oxo-piperazine-1-carboxylate (4, 900 mg, 1.59 mmol, 68% yield) as brown gummy solid.

LCMS (ES+): m/z 458.2[M+H]⁺

### Step 3: 3-(3-methyl-2-oxo-5-(2-oxopiperazin-1-yl)-2,3-dihydro-1H-benzo[d]imidazol-1-yl)piperidine-2,6-dione (5)

Into a 100 mL single-neck round-bottom flask containing a well-stirred solution of tert-butyl 4-[1-(2,6-dioxo-3-piperidyl)-3-methyl-2-oxo-benzimidazol-5-yl]-3-oxo-piperazine-1-carboxylate (4, 900 mg, 1.59 mmol) in dry DCM (20 mL) was added TFA (1.81 g, 15.86 mmol, 1.22 mL) at 0° C. The resultant reaction mixture was stirred for 3 h at room temperature. The mixture was concentrated and the resultant crude was washed with MTBE (20 mL) to afford 3-[3-methyl-2-oxo-5-(2-oxopiperazin-1-yl)benzimidazol-1-yl]piperidine-2,6-dione (5, 650 mg, 1.37 mmol, 75% yield, TFA salt) as pale pink solid.

LCMS (ES+): m/z 358.2 [M+H]⁺

### Step 4: tert-butyl 2-(4-(1-(2,6-dioxopiperidin-3-yl)-3-methyl-2-oxo-2,3-dihydro-1H-benzo[d]imidazol-5-yl)-3-oxopiperazin-1-yl)acetate (6)

Into a 100 mL single-neck round-bottom flask containing a well-stirred solution of 3-[3-methyl-2-oxo-5-(2-oxopiperazin-1-yl)benzimidazol-1-yl]piperidine-2,6-dione (5, 600 mg, 980.09 μmol, TFA salt) in anhydrous DMF (5 mL) at 0° C. under nitrogen atmosphere were added tert-butyl bromoacetate (286.75 mg, 1.47 mmol, 215.60 μL) followed by DIPEA (633.33 mg, 4.90 mmol, 853.55 uL). The resulting reaction mixture was stirred at room temperature for 3 h. The mixture was concentrated under reduced pressure to get the crude material that was purified by reverse-phase column chromatography [Column: Redisep-C18-120 g; Mobile Phase A: 0.1% Formic acid in water and Mobile Phase B: CH₃CN] to afford tert-butyl 2-[4-[1-(2,6-dioxo-3-piperidyl)-3-methyl-2-oxo-benzimidazol-5-yl]-3-oxo-piperazin-1-yl]acetate (6, 350 mg, 613.80 μmol, 63% yield, formic acid salt) as white solid.

LCMS (ES+): m/z 472.2 [M+H]⁺

### Step 5: 2-(4-(1-(2,6-dioxopiperidin-3-yl)-3-methyl-2-oxo-2,3-dihydro-1H-benzo[d]imidazol-5-yl)-3-oxopiperazin-1-yl)acetic acid (7)

Into a 100 mL single-neck round-bottom flask containing a well-stirred solution of tert-butyl 2-[4-[1-(2,6-dioxo-3-piperidyl)-3-methyl-2-oxo-benzimidazol-5-yl]-3-oxo-piperazin-1-yl]acetate (6, 330 mg, 573.88 μmol, formic acid salt) in dry 1,4-dioxane (4 mL) was added HCl (4.0 M in 1,4-dioxane, 22.96 mmol, 5.75 mL) at 0° C. The resultant reaction mixture was stirred at room temperature for 6 h at room temperature as indicated by UPLC-MS. The solvent was evaporated and the residue was washed with MTBE (20 mL) to afford 2-[4-[1-(2,6-dioxo-3-piperidyl)-3-methyl-2-oxo-benzimidazol-5-yl]-3-oxo-piperazin-1-yl]acetic acid (7, 270 mg, 529.41 μmol, 92% yield, HCl salt) as an off-white solid.

LCMS (ES+): m/z 416.0 [M+H]⁺

### 2-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)acetic acid (9)

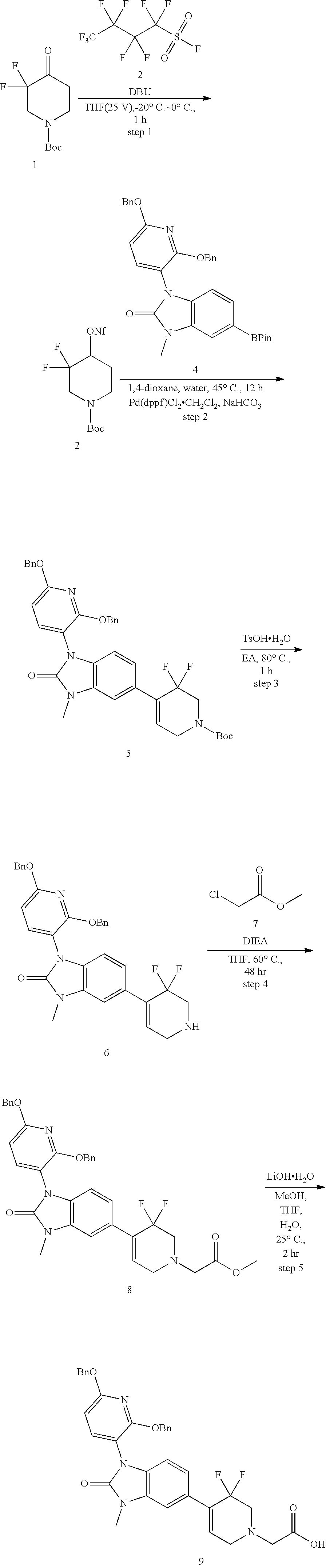

### Step 1: tert-butyl 3,3-difluoro-4-(1,1,2,2,3,3,4,4,4-nonafluorobutylsulfonyloxy)-2,6-dihydropyridine-1-carboxylate (3)

To a solution of tert-butyl 3,3-difluoro-4-oxo-piperidine-1-carboxylate (1, 10 g, 42.51 mmol) in THF (250 mL) was added DBU (19.42 g, 127.54 mmol, 19.04 mL) and 1,1,2,2,3,3,4,4,4-nonafluorobutane-1-sulfonyl fluoride (2, 38.53 g, 127.54 mmol, 22.02 mL) at −20° C. via dropwise addition under N₂. The mixture was stirred at 0° C. for 2 h. The reaction mixture was quenched by brine (500 mL) at 0° C. The aqueous layer was extracted with EA (500 mL*3). The combined organic layer was dried over Na₂SO₄, filtered and the filtrate was concentrated to get a residue. The residue was dissolved by PE:EA=10:1 (1000 mL) then filtered through silica gel (50 g) and concentrated to get tert-butyl 3,3-difluoro-4-(1,1,2,2,3,3,4,4,4-nonafluorobutylsulfonyloxy)-2,6-dihydropyridine-1-carboxylate (3, 22 g, 40.40 mmol, 95% yield) as yellow oil.

¹HNMR (400 MHz, DMSO-d₆) δ 6.83 (br s, 1H), 4.24 (br d, J=3.6 Hz, 2H), 4.09-4.03 (m, 2H), 1.42 (s, 9H).

### Step 2: tert-butyl 4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridine-1 (2H)-carboxylate (5)

To a solution of 1-(2,6-bis(benzyloxy)pyridin-3-yl)-3-methyl-5-(4,4,5,5-tetramethyl-1,3,2-dioxaborolan-2-yl)-1H-benzo[d]imidazol-2 (3H)-one (4, 4 g, 7.10 mmol), tert-butyl 3,3-difluoro-4-(1,1,2,2,3,3,4,4,4-nonafluorobutylsulfonyloxy)-2,6-dihydropyridine-1-carboxylate (3, 4.00 g, 7.73 mmol), Pd(dppf)Cl₂·CH₂Cl₂ (580.00 mg, 710.23 μmol) and NaHCO₃ (1.20 g, 14.28 mmol) in dioxane (50 mL) and H₂O (12 mL) was degassed and purged with N₂ for 3 times, and then the mixture was stirred at 45° C. for 12 hr under N₂ atmosphere. The reaction mixture was filtered and purified by reversed phase HPLC (FA) and then lyophilization. The tert-butyl 4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridine-1 (2H)-carboxylate (5, 4 g, 5.62 mmol, 79% yield) as yellow solid.

LCMS (ESI): m/z 655.2 [M+H]⁺

### Step 3: 1-(2,6-bis(benzyloxy)pyridin-3-yl)-5-(3,3-difluoro-1,2,3,6-tetrahydropyridin-4-yl)-3-methyl-1H-benzo[d]imidazol-2 (3H)-one (6)

To a mixture of tert-butyl 4-[1-(2,6-dibenzyloxy-3-pyridyl)-3-methyl-2-oxo-benzimidazol-5-yl]-3,3-difluoro-2,6-dihydropyridine-1-carboxylate (5, 2 g, 3.05 mmol) in EtOAc (20 mL) was added P-TOLUENESULFONIC ACID MONOHYDRATE (1.16 g, 6.11 mmol, 937.23 uL). The mixture was stirred at 80° C. for 1 h. The reaction mixture was quenched by addition of saturated NaHCO₃ (5 mL) aqueous at 0° C. filtered and concentrated in vacuum. The residue was then purified by reversed phase HPLC (FA) and then lyophilization. The 1-(2,6-bis(benzyloxy)pyridin-3-yl)-5-(3,3-difluoro-1,2,3,6-tetrahydropyridin-4-yl)-3-methyl-1H-benzo[d]imidazol-2 (3H)-one (6, 1.4 g, 2.10 mmol, 69% yield) as yellow solid was obtained.

LCMS (ESI): m/z 555.1 [M+H]⁺

### Step 4: methyl 2-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)acetate (8)

To a mixture of 1-(2,6-dibenzyloxy-3-pyridyl)-5-(3,3-difluoro-2,6-dihydro-1H-pyridin-4-yl)-3-methyl-benzimidazol-2-one (6, 1 g, 1.80 mmol) and methyl 2-chloroacetate (7, 1.96 g, 18.03 mmol, 1.58 mL) in THF (10 mL) was added DIPEA (139.83 mg, 1.08 mmol, 188.45 uL). The mixture was stirred at 60° C. for 48 h. The reaction mixture was quenched by water (20 mL) and extracted with EtOAc (20 mL*2) and concentrated to get methyl 2-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)acetate (8, 1.1 g, 1.19 mmol, 66% yield) as yellow solid.

LCMS (ESI): m/z 627.1 [M+H]⁺

### Step 5: 2-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)acetic acid (9)

To a solution of methyl 2-[4-[1-(2,6-dibenzyloxy-3-pyridyl)-3-methyl-2-oxo-benzimidazol-5-yl]-3,3-difluoro-2,6-dihydropyridin-1-yl]acetate (8, 1.1 g, 1.76 mmol) in THF (5 mL) and Methanol (5 mL) was added a solution of LiOH·H₂O (368.31 mg, 8.78 mmol) in Water (5 mL). The mixture was stirred at 25° C. for 2 h. The reaction was quenched by 1N HCl and adjust pH to 6˜7 then concentrated to get a residue. The residue was purified by reversed phase HPLC (FA) and then lyophilization. The 2-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)acetic acid (9, 520 mg, 814.86 μmol, 46% yield) as yellow solid.

LCMS (ESI): m/z 613.2 [M+H]⁺

### 2-(1-(1-(2,6-bis(benzyloxy)pyridin-3-yl)-3-methyl-2-oxo-2,3-dihydro-1H-benzo[d]imidazol-5-yl)piperidin-4-yl)-2-methylpropanamide (8)

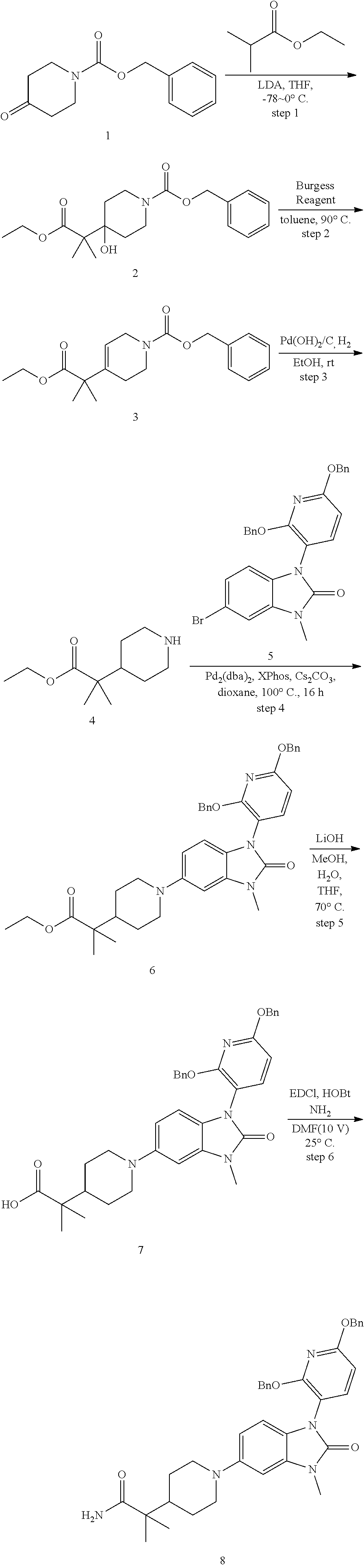

### Step 1: benzyl 4-(1-ethoxy-2-methyl-1-oxopropan-2-yl)-4-hydroxypiperidine-1-carboxylate (2)

To a solution of (diisopropylamino)lithium (2 M in THF, 12.86 mL) in THF (40 mL) was added ethyl isobutyrate (2.74 g, 23.58 mmol) in THF (15 mL) at −78° C. under N₂. After stirring at −78˜0° C. for 1 h, a solution of benzyl 4-oxopiperidine-1-carboxylate (1, 5 g, 21.44 mmol) in THF (40 mL) was added at −78° C. The mixture was stirred at −78˜0° C. for 2 h. The reaction mixture was poured into 1N HCl (50 mL) at 0° C., and then extracted with ethyl acetate (50 mL×3). The combined organic layers were washed with brine (30 mL×2), dried over Na₂SO₄, filtered and concentrated under reduced pressure. The residue was purified by column chromatography (SiO₂, Petroleum ether/Ethyl acetate=50/1 to 5/1) to afford benzyl 4-(1-ethoxy-2-methyl-1-oxopropan-2-yl)-4-hydroxypiperidine-1-carboxylate (2, 6.5 g, 18.34 mmol, 85% yield) as a yellow oil.

LCMS (ESI): m/z 350.2 [M+H]⁺

### Step 2: benzyl 4-(1-ethoxy-2-methyl-1-oxopropan-2-yl)-5,6-dihydropyridine-1 (2H)-carboxylate (3)

A mixture of benzyl 4-(1-ethoxy-2-methyl-1-oxopropan-2-yl)-4-hydroxypiperidine-1-carboxylate (2, 6 g, 17.17 mmol) and methoxycarbonyl-(triethylammonio)sulfonyl-azanide (6.14 g, 25.76 mmol) in toluene (150 mL) was stirred at 90° C. for 12 h. The reaction mixture was concentrated under reduced pressure to remove toluene. The residue was purified by column chromatography (SiO₂, Petroleum ether/Ethyl acetate=20/1 to 5/1) to afford benzyl 4-(1-ethoxy-2-methyl-1-oxopropan-2-yl)-5,6-dihydropyridine-1 (2H)-carboxylate (3, 5 g, 15.09 mmol, 87% yield) as a yellow oil.

¹H NMR (400 MHz, CDCl3) δ=7.41-7.29 (m, 5H), 5.64-5.46 (m, 1H), 5.16 (s, 2H), 4.17-4.08 (m, 2H), 4.02 (q, J=2.4 Hz, 2H), 3.55 (br t, J=5.4 Hz, 2H), 2.08 (s, 2H), 1.31 (s, 6H), 1.23 (t, J=7.1 Hz, 3H).

### Step 3: ethyl 2-methyl-2-(piperidin-4-yl)propanoate (4)

To a solution of benzyl 4-(1-ethoxy-2-methyl-1-oxopropan-2-yl)piperidine-1-carboxylate (3, 0.5 g, 1.50 mmol) in Ethanol (160 mL) was added Pd(OH)₂/C (0.1 g, 1.50 mmol, 10% purity) under N₂ atmosphere. The suspension was degassed and purged with H₂ for 3 times. The mixture was stirred under H₂ (15 Psi) at 30° C. for 12 h. The reaction mixture was filtered and concentrated under reduced pressure to afford ethyl 2-methyl-2-(piperidin-4-yl)propanoate (4, 0.3 g, 1.35 mmol, 90% yield) as yellow oil. The material was used in the next step without further purification.

¹H NMR (400 MHz, CDCl3) δ=4.20-4.05 (m, 2H), 3.13 (br d, J=12.5 Hz, 2H), 2.53 (t, J=11.2 Hz, 2H), 1.77-1.71 (m, 1H), 1.53 (br d, J=12.5 Hz, 2H), 1.33-1.18 (m, 5H), 1.22 (s, 6H).

### Step 4: ethyl 2-(1-(1-(2,6-bis(benzyloxy)pyridin-3-yl)-3-methyl-2-oxo-2,3-dihydro-1H-benzo[d]imidazol-5-yl)piperidin-4-yl)-2-methylpropanoate (6)

A mixture of ethyl 2-methyl-2-(piperidin-4-yl)propanoate (4, 231.56 mg, 1.16 mmol), 1-(2,6-bis(benzyloxy)pyridin-3-yl)-5-bromo-3-methyl-1H-benzo[d]imidazol-2 (3H)-one (5, 500 mg, 968.27 μmol), dicesium; carbonate (630.96 mg, 1.94 mmol), dicyclohexyl-[2-(2,4,6-triisopropylphenyl)phenyl]phosphane (92.32 mg, 193.65 μmol) and (1E,4E)-1,5-diphenylpenta-1,4-dien-3-one; palladium (88.67 mg, 96.83 μmol) in dioxane (5 mL) was degassed and purged with N₂ (3 times), and then the mixture was stirred at 100° C. for 12 h under N₂ atmosphere. The reaction mixture was diluted with water (30 mL) and extracted with EA (30 mL*3). The combined with organic phase was dried by anhydrous Na₂SO₄ filtered and concentrated under vacuum. The residue was purified on automated flash chromatography system (ethyl acetate/petroleum ether from 10:1 to 1:1) and concentrated under vacuum. The crude of ethyl 2-(1-(1-(2,6-bis(benzyloxy)pyridin-3-yl)-3-methyl-2-oxo-2,3-dihydro-1H-benzo[d]imidazol-5-yl)piperidin-4-yl)-2-methylpropanoate (6, 0.44 g, 658.51 μmol, 68% yield).

LCMS (ESI): m/z 635.0 [M+H]⁺

### Step 5: 2-(1-(1-(2,6-bis(benzyloxy)pyridin-3-yl)-3-methyl-2-oxo-2,3-dihydro-1H-benzo[d]imidazol-5-yl)piperidin-4-yl)-2-methylpropanoic acid (7)

To a solution of ethyl 2-(1-(1-(2,6-bis(benzyloxy)pyridin-3-yl)-3-methyl-2-oxo-2,3-dihydro-1H-benzo[d]imidazol-5-yl)piperidin-4-yl)-2-methylpropanoate (6, 0.44 g, 693.17 μmol) in THF (3 mL), Methanol (3 mL) and Water (3 mL) was added lithium; hydroxide; hydrate (145.44 mg, 3.47 mmol, 96.32 uL). The mixture was stirred at 70° C. for 24 hrs. The reaction mixture was concentrated under vacuum. The residue was adjusted to pH=3 and extracted with DCM (25 mL*3). The combined organic layers were washed with brine (25 mL), dried over Na₂SO₄, filtered and concentrated under vacuum. The residue was purified by reversed-phase column (0.1% FA). The desired fraction was collected and dried by lyophilization. Compound 2-(1-(1-(2,6-bis(benzyloxy)pyridin-3-yl)-3-methyl-2-oxo-2,3-dihydro-1H-benzo[d]imidazol-5-yl)piperidin-4-yl)-2-methylpropanoic acid (7, 290 mg, 477.99 μmol, 69% yield) as yellow solid.

LCMS (ESI): m/z 607.2 [M+H]⁺

### Step 6: 2-(1-(1-(2,6-bis(benzyloxy)pyridin-3-yl)-3-methyl-2-oxo-2,3-dihydro-1H-benzo[d]imidazol-5-yl)piperidin-4-yl)-2-methylpropanamide (8)

To a solution of 2-(1-(1-(2,6-bis(benzyloxy)pyridin-3-yl)-3-methyl-2-oxo-2,3-dihydro-1H-benzo[d]imidazol-5-yl)piperidin-4-yl)-2-methylpropanoic acid (7, 230 mg, 379.09 μmol) in DMF (2.3 mL) were added EDCI (109.01 mg, 568.64 μmol), HOBt·NH₃ (86.52 mg, 568.64 μmol) and DIPEA (146.99 mg, 1.14 mmol, 198.09 uL). The mixture was stirred at 60° C. for 2 h. The reaction was quenched by water 10 (mL), extracted with EtOAc (10 mL*5) dried over Na₂SO₄ and concentrated to a residue. The residue was trituration with PE:EA=3:1 (5 mL). The 2-(1-(1-(2,6-bis(benzyloxy)pyridin-3-yl)-3-methyl-2-oxo-2,3-dihydro-1H-benzo[d]imidazol-5-yl)piperidin-4-yl)-2-methylpropanamide (8, 190 mg, 279.17 μmol, 74% yield) as white solid.

LCMS (ESI): m/z 606.42 [M+H]⁺

### 2-(1-(1-(2,6-bis(benzyloxy)pyridin-3-yl)-3-methyl-2-oxo-2,3-dihydro-1H-benzo[d]imidazol-5-yl)-1H-indol-5-yl)acetic acid (6)

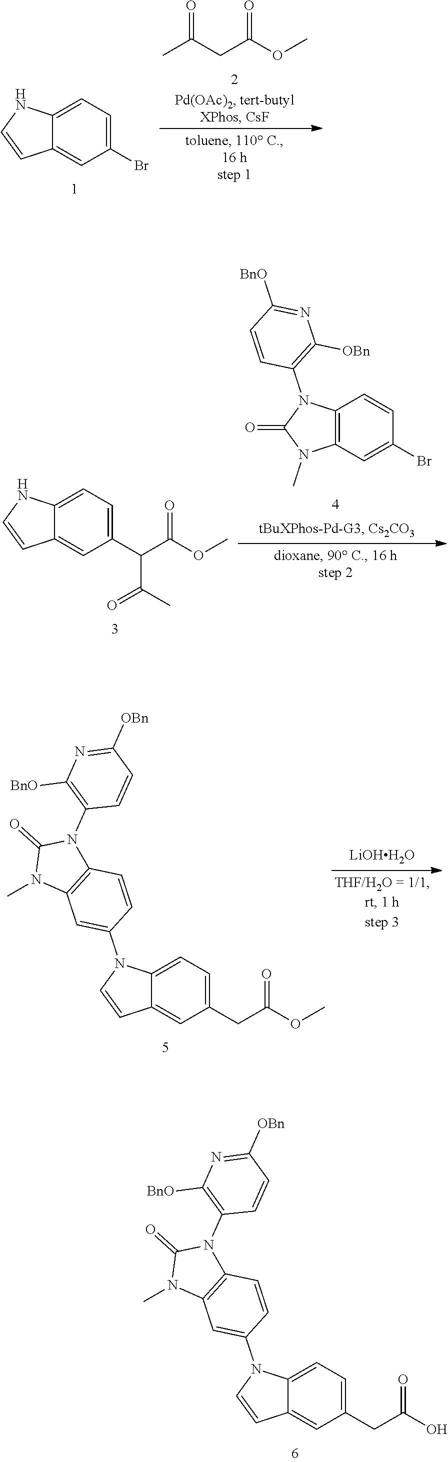

### Step 1: methyl 2-(1H-indol-5-yl)-3-oxo-butanoate (3)

A mixture of 5-bromo-1H-indole (1, 10 g, 51.01 mmol), 5-bromo-1H-indole (2, 10 g, 51.01 mmol), methyl 3-oxobutanoate (5.92 g, 51.01 mmol, 5.48 mL), tert-Butyl XPhos (2.17 g, 5.10 mmol), fluorocesium (30.99 g, 204.04 mmol, 7.52 mL) and diacetoxypalladium (572.60 mg, 2.55 mmol) in Toluene (100 mL) was de gassed and purged with N₂ 3 times, and then the mixture was stirred at 110° C. for 16 hr under N₂ atmosphere. The reaction mixture was poured into water (200 mL) and extracted with ethyl acetate (2*200 mL). Organic phases were combined and washed with brine (200 mL), dried by anhydrous Na₂SO₄, filtered and concentrated in vacuum. The residue was purified by flash silica gel chromatography (Biotage®; 120 g SepaFlash® Silica Flash Column, Eluent of 0-50% Ethylacetate/Petroleum ether gradient @ 100 mL/min). Compound methyl 2-(1H-indol-5-yl)-3-oxo-butanoate (3, 2.6 g, 10.08 mmol, 20% yield) as yellow oil.

LCMS (ESI): m/z 232.0 [M+H]⁺

### Step 2: methyl 2-(1-(1-(2,6-bis(benzyloxy)pyridin-3-yl)-3-methyl-2-oxo-2,3-dihydro-1H-benzo[d]imidazol-5-yl)-1H-indol-5-yl)acetate (5)

To a mixture of 1-(2,6-bis(benzyloxy)pyridin-3-yl)-5-bromo-3-methyl-1H-benzo[d]imidazol-2 (3H)-one (4, 1 g, 1.94 mmol), methyl 2-(1H-indol-5-yl)-3-oxobutanoate (3, 500.00 mg, 2.16 mmol), tBuXPhos-Pd-G3 (153.83 mg, 193.65 μmol) and Cs₂CO₃ (1.89 g, 5.81 mmol) was added dioxane (10 mL). The mixture was stirred at 90° C. for 16 h under N₂. The reaction mixture was concentrated under vacuum. The residue was purified by flash silica gel chromatography (flow: 50 mL/min; gradient: 0-100% ethylacetate in petroleum ether; ISCO®; 40 g SepaFlash®Silica FlashColumn; ethylacetate/petroleumether=1/0) to afford methyl 2-(1-(1-(2,6-bis(benzyloxy)pyridin-3-yl)-3-methyl-2-oxo-2,3-dihydro-1H-benzo[d]imidazol-5-yl)-1H-indol-5-yl)acetate (5, 860 mg, 1.00 mmol, 52% yield) as yellow solid.

LCMS (ESI): m/z 625.2 [M+H]⁺

### Step 3: 2-(1-(1-(2,6-bis(benzyloxy)pyridin-3-yl)-3-methyl-2-oxo-2,3-dihydro-1H-benzo[d]imidazol-5-yl)-1H-indol-5-yl)acetic acid (6)

To a solution of methyl 2-[1-[1-(2,6-dibenzyloxy-3-pyridyl)-3-methyl-2-oxo-benzimidazol-5-yl]indol-5-yl]acetate (5, 860 mg, 1.38 mmol) in THF (3 mL) and Methanol (3 mL) was added a solution of LiOH·H₂O (173.31 mg, 4.13 mmol) in Water (3 mL). The mixture was stirred at 25° C. for 2 h. The reaction was quenched by 1N HCl and adjusted pH to 6˜7 then concentrated to get a residue. The residue was purified by reversed phase HPLC (FA) and then lyophilization. The 2-(1-(1-(2,6-bis(benzyloxy)pyridin-3-yl)-3-methyl-2-oxo-2,3-dihydro-1H-benzo[d]imidazol-5-yl)-1H-indol-5-yl)acetic acid (6, 320 mg, 513.55 μmol, 37% yield) as yellow solid.

LCMS (ESI): m/z 611.1 [M+H]⁺

### 1-(2,6-bis(benzyloxy)pyridin-3-yl)-3-methyl-5-(4,4,5,5-tetramethyl-1,3,2-dioxaborolan-2-yl)-1H-benzo[d]imidazol-2 (3H)-one (2)

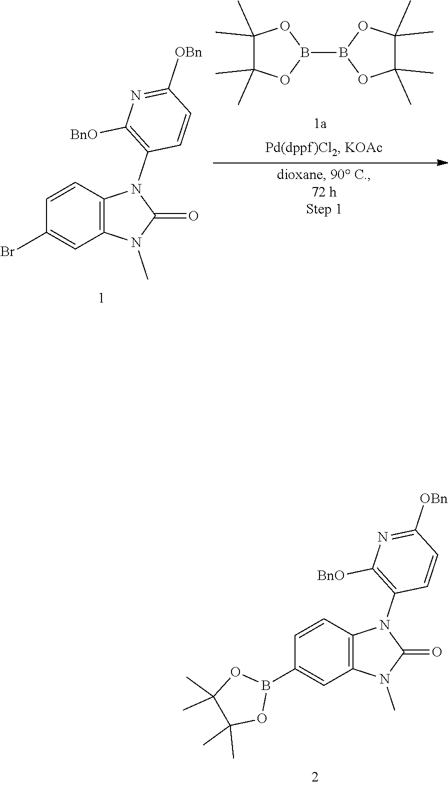

### Step 1:1-(2,6-bis(benzyloxy)pyridin-3-yl)-3-methyl-5-(4,4,5,5-tetramethyl-1,3,2-dioxaborolan-2-yl)-1H-benzo[d]imidazol-2 (3H)-one (2)

To a solution of 5-bromo-1-(2,6-dibenzyloxy-3-pyridyl)-3-methyl-benzimidazol-2-one (1, 10 g, 19.37 mmol) and 4,4,5,5-tetramethyl-2-(4,4,5,5-tetramethyl-1,3,2-dioxaborolan-2-yl)-1,3,2-dioxaborolane (1a, 9.84 g, 38.73 mmol) in dioxane (30 mL) was added KOAc (5.70 g, 58.10 mmol) and Pd(dppf)Cl₂ (1.42 g, 1.94 mmol). The mixture was stirred at 90° C. for 1 hr under N₂. The reaction mixture was filtered and concentrated under reduced pressure. The reduced was dissolved with EtOAc (30 mL) and filtered through silica gel (10 g) and concentrated to get a residue. The residue was triturated with MTBE (10 mL) at 25° C. for 10 mins and filtered, the filter cake was concentrated at 40° C. under vacuum to get product without further purification. The 1-(2,6-bis(benzyloxy)pyridin-3-yl)-3-methyl-5-(4,4,5,5-tetramethyl-1,3,2-dioxaborolan-2-yl)-1H-benzo[d]imidazol-2 (3H)-one (2, 9 g, 15.01 mmol, 78% yield) as yellow solid.

LCMS (ESI): m/z 564.2 [M+H]⁺

¹H NMR (400 MHz, DMSO-d₆) δ 7.81 (d, J=8.4 Hz, 1H), 7.48-7.20 (m, 12H), 6.71 (d, J=7.8 Hz, 1H), 6.62 (d, J=8.4 Hz, 1H), 5.46-5.30 (m, 4H), 3.41 (s, 3H), 1.30 (s, 12H).

### (S)-1-(2,6-bis(benzyloxy)pyridin-3-yl)-3-methyl-5-(6-methyl-1,2,3,6-tetrahydropyridin-4-yl)-1H-benzo[d]imidazol-2 (3H)-one (9)

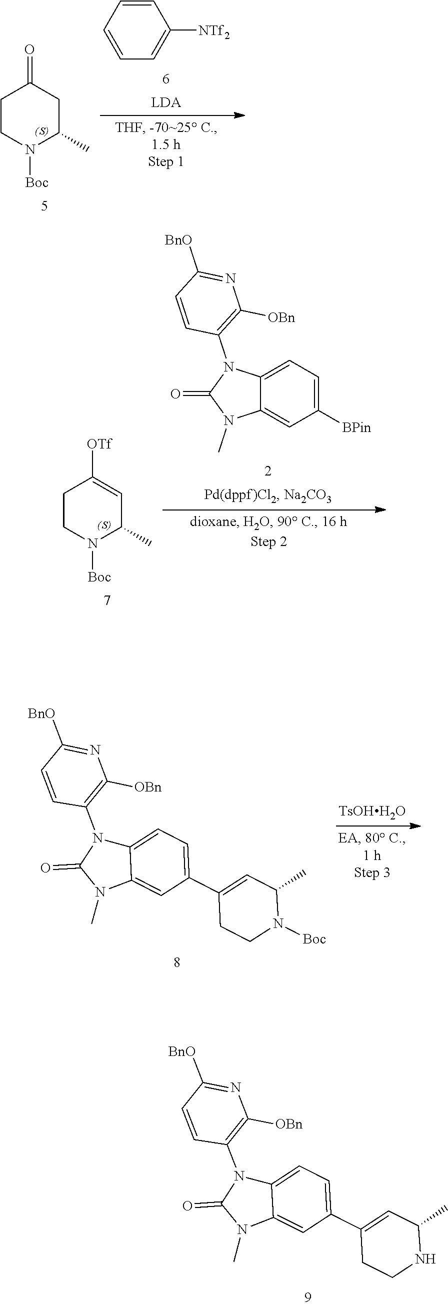

### Step 1: (S)-tert-butyl 2-methyl-4-(((trifluoromethyl)sulfonyl)oxy)-5,6-dihydropyridine-1 (2H)-carboxylate (7)

To a solution of (S)-tert-butyl 2-methyl-4-oxopiperidine-1-carboxylate (5, 2 g, 9.38 mmol) in THF (20 mL) was added LDA (2 M, 5.58 mL, in THF) dropwise at −70° C. The mixture was stirred at −70° C. for 0.5 h. Then a solution of 1,1,1-trifluoro-N-phenyl-N-((trifluoromethyl)sulfonyl)methanesulfonamide (6, 4.02 g, 11.25 mmol) in THF (5 mL) was added dropwise at −70° C. The mixture was gradually raised to 25° C. The mixture was stirred at 25° C. for 1 h under N₂. The reaction mixture was quenched with saturated NH₄Cl (50 mL). The mixture was diluted with EtOAc (150 mL) and water (150 mL). The layers were separated and the aqueous phase was extracted with EtOAc (150 mL×3). Combined organic phase were washed with brine (150 mL), dried over Na₂SO₄, filtered and concentrated under reduced pressure. The residue was purified by column chromatography (SiO₂, Petroleum ether:Ethyl acetate=1:0 to 5:1) to afford (S)-tert-butyl 2-methyl-4-(((trifluoromethyl)sulfonyl)oxy)-5,6-dihydropyridine-1 (2H)-carboxylate (7, 3 g, 8.69 mmol, 93% yield) as a colorless oil.

### Step 2: (S)-tert-butyl 4-(1-(2,6-bis(benzyloxy)pyridin-3-yl)-3-methyl-2-oxo-2,3-dihydro-1H-benzo[d]imidazol-5-yl)-2-methyl-5,6-dihydropyridine-1 (2H)-carboxylate (8)

To a mixture of (S)-tert-butyl 2-methyl-4-(((trifluoromethyl)sulfonyl)oxy)-5,6-dihydropyridine-1 (2H)-carboxylate (7, 500 mg, 1.45 mmol), 1-(2,6-bis(benzyloxy)pyridin-3-yl)-3-methyl-5-(4,4,5,5-tetramethyl-1,3,2-dioxaborolan-2-yl)-1H-benzo[d]imidazol-2 (3H)-one (2, 978.96 mg, 1.74 mmol), Pd(dppf)Cl₂ (105.94 mg, 144.79 μmol) and Na₂CO₃ (460.37 mg, 4.34 mmol, 181.97 μL) in dioxane (8 mL) and H₂O (2 mL) under N₂. The mixture was stirred at 90° C. for 12 h under N₂. The reaction mixture was concentrated under vacuum. The residue was purified by flash silica gel chromatography (flow: 50 mL/min; gradient: 0-100% ethylacetate in petroleum ether; ISCO®; 40 g SepaFlash®Silica Flash Column; ethylacetate/petroleumether=1/0) to afford (S)-tert-butyl 4-(1-(2,6-bis(benzyloxy)pyridin-3-yl)-3-methyl-2-oxo-2,3-dihydro-1H-benzo[d]imidazol-5-yl)-2-methyl-5,6-dihydropyridine-1 (2H)-carboxylate (8, 530 mg, 762.23 μmol, 53% yield) as a yellow solid.

LCMS (ESI): m/z 633.3 [M+H]⁺

### Step 3: (S)-1-(2,6-bis(benzyloxy)pyridin-3-yl)-3-methyl-5-(6-methyl-1,2,3,6-tetrahydropyridin-4-yl)-1H-benzo[d]imidazol-2 (3H)-one (9)

To a mixture of (S)-tert-butyl 4-(1-(2,6-bis(benzyloxy)pyridin-3-yl)-3-methyl-2-oxo-2,3-dihydro-1H-benzo[d]imidazol-5-yl)-2-methyl-5,6-dihydropyridine-1 (2H)-carboxylate (8, 200 mg, 316.08 μmol) in EtOAc (2 mL) was added P-TOLUENESULFONIC ACID MONOHYDRATE (120.25 mg, 632.16 μmol, 96.97 uL). The mixture was stirred at 80° C. for 1 h. The reaction mixture was concentrated under vacuum. The residue was purified by reversed phase HPLC (FA) and then lyophilisation to afford (S)-1-(2,6-bis(benzyloxy)pyridin-3-yl)-3-methyl-5-(6-methyl-1,2,3,6-tetrahydropyridin-4-yl)-1H-benzo[d]imidazol-2 (3H)-one (9, 150 mg, 238.48 μmol, 75% yield, formic acid salt) as an off-white solid.

LCMS (ESI): m/z 533.2 [M+H]⁺

### 2-[4-[3-(2,6-dioxo-3-piperidyl)-1-methyl-2-oxo-imidazo[4,5-c]pyridin-6-yl]phenyl]acetic acid (8)

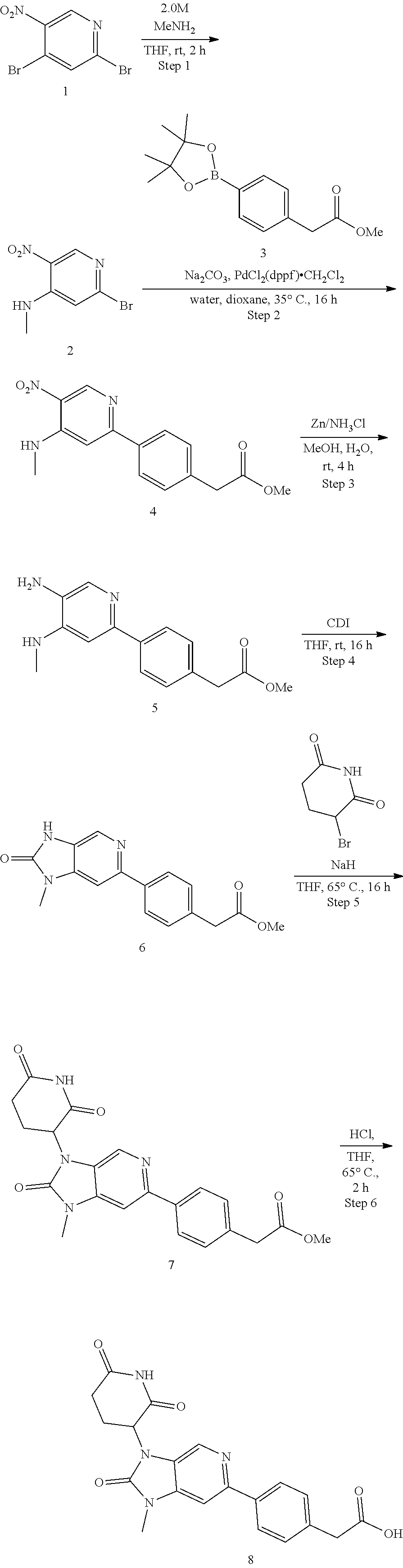

### Step 1: 2-bromo-N-methyl-5-nitro-pyridin-4-amine (2)

Into a 500 mL three-neck round-bottom flask containing a well-stirred solution of 2,4-dibromo-5-nitro-pyridine (1, 5 g, 17.74 mmol) in dry THF (10 mL) was added methylamine (2.0 M solution in THF, 35.47 mmol, 17.5 mL) under nitrogen atmosphere at room temperature and the reaction mixture was stirred for 2 h. Afterwards, saturated sodium chloride solution (100 mL) was added and extracted with EtOAc (2×200 mL). The combined organic layers were dried over sodium sulfate and concentrated under reduced pressure to get the crude material that was purified by silica gel flash column chromatography (100-200 mesh silica gel; 30-40% EtOAc in pet ether) to afford 2-bromo-N-methyl-5-nitro-pyridin-4-amine (2, 3.5 g, 14.33 mmol, 81% yield) as a yellow solid.

LCMS (ES+): m/z 232.21 [M+H]⁺

### Step 2: 2-[4-[4-(methylamino)-5-nitro-2-pyridyl]phenyl]acetate (4)

Into a 250 mL sealed-tube containing a well-stirred solution of 2-bromo-N-methyl-5-nitro-pyridin-4-amine (2, 3.5 g, 14.33 mmol) and methyl 2-[4-(4,4,5,5-tetramethyl-1,3,2-dioxaborolan-2-yl)phenyl]acetate (3, 6.21 g, 21.49 mmol) and sodium carbonate (4.56 g, 42.99 mmol) in 1,4-dioxane (50 mL) and water (5 mL), nitrogen gas was purged for 5 min. Afterwards, Pd(dppf)Cl₂·DCM (1.17 g, 1.43 mmol) was added and the resulting mixture was heated at 85° C. for 16 h. Subsequently, the reaction mixture was filtered through Celite bed and washed with 10% MeOH in DCM (250 mL). The filtrate was concentrated under reduced pressure to get the crude material that was purified by silica gel flash column chromatography (100-200 mesh silica gel; 80-90% EtOAc in pet ether) to afford methyl 2-[4-[4-(methylamino)-5-nitro-2-pyridyl]phenyl]acetate (4, 3.2 g, 9.07 mmol, 63% yield) as a yellow solid.

LCMS (ESI) m/z 302.2 [M+H]⁺

### Step 3: methyl 2-[4-[5-amino-4-(methylamino)-2-pyridyl]phenyl]acetate (5)

Into a 250 mL single-neck round-bottom flask containing a well-stirred solution of methyl 2-[4-[4-(methylamino)-5-nitro-2-pyridyl]phenyl]acetate (4, 3.40 g, 9.63 mmol) in methanol (30 mL) and water (15 mL) were added zinc powder (325 mesh-high grade material, 3.15 g, 48.17 mmol) and ammonium chloride (2.58 g, 48.17 mmol) at room temperature and the resulting mixture was stirred for 4 h. The mixture was filtered through Celite and washed with 10% methanol in DCM (300 mL). The filtrate was dried over sodium sulfate and concentrated under reduced pressure to get methyl 2-[4-[5-amino-4-(methylamino)-2-pyridyl]phenyl]acetate (5, 2.7 g, 9.56 mmol, 99% yield) as a dark brown solid.

LCMS (ESI) m/z 272.2 [M+H]⁺

### Step 4: methyl 2-[4-(1-methyl-2-oxo-3H-imidazo[4,5-c]pyridin-6-yl)phenyl]acetate (6)

Into a 100 mL single-neck round-bottom flask containing a well-stirred solution of methyl 2-[4-[5-amino-4-(methylamino)-2-pyridyl]phenyl]acetate (5, 2.7 g, 9.63 mmol) in dry THF (80 mL) was added 1,1′-carbonyldiimidazole (3.90 g, 24.08 mmol) under nitrogen atmosphere at ambient temperature and the resulting mixture was stirred for 16 h at room temperature. The solvent was evaporated under reduced pressure to get a residue that was purified by silica gel flash column chromatography (230-400 mesh silica gel; 10% MeOH in DCM) to afford methyl 2-[4-(1-methyl-2-oxo-3H-imidazo[4,5-c]pyridin-6-yl)phenyl]acetate (6, 1.3 g, 4.36 mmol, 45% yield) as a light pink solid.

LCMS (ES+): m/z 298.2 [M+H]⁺

### Step 5: methyl 2-[4-[3-(2,6-dioxo-3-piperidyl)-1-methyl-2-oxo-imidazo[4,5-c]pyridin-6-yl]phenyl]acetate (7)

Into a 100 mL three-neck round-bottom flask containing a well-stirred solution of methyl 2-[4-(1-methyl-2-oxo-3H-imidazo[4,5-c]pyridin-6-yl)phenyl]acetate (6, 500 mg, 1.68 mmol) in dry THF (50 mL) was added sodium hydride (60% dispersion in mineral oil, 642.91 mg, 16.78 mmol, 60% purity) at 0° C. and the reaction mixture was stirred at ambient temperature. After 1 h, 3-bromopiperidine-2,6-dione (1.36 g, 6.71 mmol) dissolved in dry THF (7 mL) was added at 0° C. The reaction mixture was then heated at 65° C. for 16 h. Afterwards, saturated ammonium chloride solution (5 mL) was added to the reaction mixture at 0° C. and extracted with EtOAc (3×50 mL). The combined organic layers were dried over sodium sulfate and concentrated under reduced pressure to get methyl 2-[4-[3-(2,6-dioxo-3-piperidyl)-1-methyl-2-oxo-imidazo[4,5-c]pyridin-6-yl]phenyl]acetate (7, 500 mg, 890.53 μmol, 53% yield) as a dark brown solid.

LCMS (ES+): m/z 409.2 [M+H]⁺

### Step 6: 2-[4-[3-(2,6-dioxo-3-piperidyl)-1-methyl-2-oxo-imidazo[4,5-c]pyridin-6-yl]phenyl]acetic acid (8)

Into a 100 mL sealed-tube containing a well-stirred solution of methyl 2-[4-[3-(2,6-dioxo-3-piperidyl)-1-methyl-2-oxo-imidazo[4,5-c]pyridin-6-yl]phenyl]acetate (7, 500 mg, 890.53 μmol) in THF (10 mL) was added 1.5 M Hydrochloride solution (30 mmol, 20 mL) and the resulting mixture was heated at 65° C. for 2 h. The solvent was evaporated under vacuum to get the crude material that was purified by reverse-phase preparative HPLC [Column: Redisep C18 (150×19) mm, 5 μm; Mobile phase A: 0.1% Formic acid in water and Mobile phase B: MeCN] to get 2-[4-[3-(2,6-dioxo-3-piperidyl)-1-methyl-2-oxo-imidazo[4,5-c]pyridin-6-yl]phenyl]acetic acid (8, 180 mg, 360.36 μmol, 40% yield, formic acid salt) as a brown solid.

LCMS (ES+): m/z 395.2 [M+H]⁺

### 2-[(3R)-3-[1-(2,6-dioxo-3-piperidyl)-3-methyl-2-oxo-benzimidazol-5-yl]pyrrolidin-1-yl]acetic acid (9a)

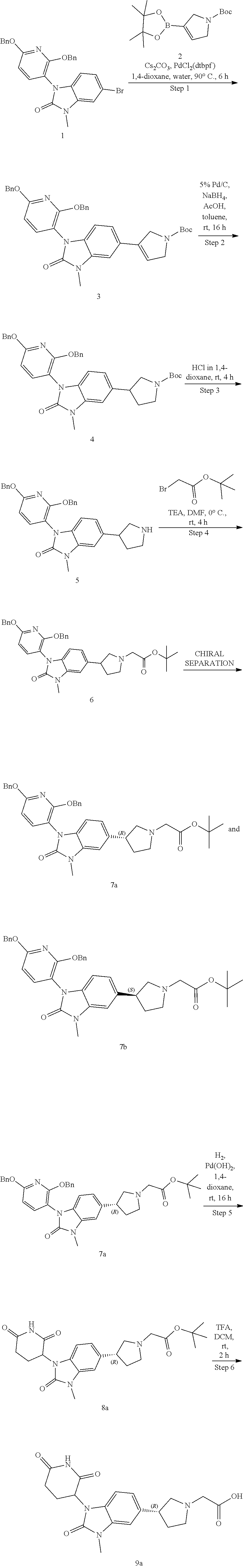

Note: Configurations are arbitrarily assigned.

### Step 1: tert-butyl 3-[1-(2,6-dibenzyloxy-3-pyridyl)-3-methyl-2-oxo-benzimidazol-5-yl]-2,5-dihydropyrrole-1-carboxylate (2)

Into a 50 mL pressure tube containing a well-stirred solution of 5-bromo-1-(2,6-dibenzyloxy-3-pyridyl)-3-methyl-benzimidazol-2-one (1, 2 g, 3.49 mmol) in anhydrous 1,4-dioxane (12 mL) and water (2 mL) were added and tert-butyl 3-(4,4,5,5-tetramethyl-1,3,2-dioxaborolan-2-yl)-2,5-dihydropyrrole-1-carboxylate (2, 1.34 g, 4.53 mmol) and Cesium carbonate (3.41 g, 10.46 mmol) at room temperature under nitrogen atmosphere. The reaction mixture was degassed by bubbling nitrogen gas through the solution for 5 min. Subsequently, 1,1′-bis(di-tert-butylphosphino)ferrocene]dichloropalladium(II) (114.53 mg, 174.29 μmol) was added to the reaction mixture and the resulting suspension was heated to 90° C. for 6 h. The reaction mixture was cooled to room temperature and poured into water (150 mL). The aqueous layer was extracted with ethyl acetate (2×300 mL). The organic layer was dried with anhydrous Na₂SO₄, filtered and concentrated under reduced pressure to get crude compound, which was purified by flash silica gel column chromatography (30-40% ethyl acetate in pet ether) to afford tert-butyl 3-[1-(2,6-dibenzyloxy-3-pyridyl)-3-methyl-2-oxo-benzimidazol-5-yl]-2,5-dihydropyrrole-1-carboxylate (3, 1.9 g, 2.98 mmol, 86% yield) as an off-white solid.

LCMS (ES+): m/z 605.2 [M+H]⁺

### Step 2: tert-butyl 3-[1-(2,6-dibenzyloxy-3-pyridyl)-3-methyl-2-oxo-benzimidazol-5-yl]pyrrolidine-1-carboxylate (4)

Into a 200 mL tinyclave pressure reactor containing a well-stirred solution of tert-butyl 3-[1-(2,6-dibenzyloxy-3-pyridyl)-3-methyl-2-oxo-benzimidazol-5-yl]-2,5-dihydropyrrole-1-carboxylate (3, 1.9 g, 2.95 mmol) in toluene (20 mL) were added acetic acid (1.01 g, 16.79 mmol, 960.15 μL) and 5 wt. % palladium on carbon, (627.34 mg, 295.36 μmol, 5% purity) followed sodium borohydride (558.67 mg, 14.77 mmol) at ambient temperature. The tinyclave was closed and stirred for 2 h. Afterwards, sodium borohydride (558.67 mg, 14.77 mmol) was added and the stirring was continued for 14 h. The reaction mixture was filtered through Celite and washed with mixture of THF (500 mL) and toluene (100 mL), followed by ethyl acetate (200 mL). The filtrate was concentrated under reduced pressure and suspended in water (300 mL). The aqueous layer was extracted with ethyl acetate (2×400 mL). The organic layer was dried with anhydrous Na₂SO₄, filtered and concentrated under reduced pressure. The crude compound was purified by flash silica gel column chromatography (40-50% ethyl acetate in pet ether) to afford tert-butyl 3-[1-(2,6-dibenzyloxy-3-pyridyl)-3-methyl-2-oxo-benzimidazol-5-yl]pyrrolidine-1-carboxylate (4, 1.1 g, 1.52 mmol, 51% yield) as a pale yellow solid.

LCMS (ES+): m/z 551.2 [M−tBu+H]⁺

### Step 3: 1-(2,6-dibenzyloxy-3-pyridyl)-3-methyl-5-pyrrolidin-3-yl-benzimidazol-2-one (5)

Into a 100 mL single-neck round-bottom flask containing a well-stirred solution of tert-butyl 3-[1-(2,6-dibenzyloxy-3-pyridyl)-3-methyl-2-oxo-benzimidazol-5-yl]pyrrolidine-1-carboxylate (4, 1.1 g, 1.50 mmol) in 1,4-dioxane (8 mL) was added a 4.0 M solution of hydrogen chloride in 1,4-dioxane (5.64 mL) at 0° C. The reaction mixture was stirred at room temperature for 4 h. The reaction mixture was concentrated under reduced pressure and the residue was triturated with diethyl ether (15 mL), filtered and dried to obtain 1-(2,6-dibenzyloxy-3-pyridyl)-3-methyl-5-pyrrolidin-3-yl-benzimidazol-2-one (5, 850 mg, 1.29 mmol, 85% yield, HCl salt) as a pale yellow gummy solid.

LCMS (ES+): m/z 507.2 [M+H]⁺

### Step 4: tert-butyl 2-[(3R)-3-[1-(2,6-dibenzyloxy-3-pyridyl)-3-methyl-2-oxo-benzimidazol-5-yl]pyrrolidin-1-yl]acetate (7a) and tert-butyl 2-[(3S)-3-[1-(2,6-dibenzyloxy-3-pyridyl)-3-methyl-2-oxo-benzimidazol-5-yl]pyrrolidin-1-yl]acetate (7b)

Into a 50 mL single neck round bottom flask containing a well-stirred solution of 1-(2,6-dibenzyloxy-3-pyridyl)-3-methyl-5-pyrrolidin-3-yl-benzimidazol-2-one (5, 850 mg, 1.48 mmol) in DMF (8 mL) was added triethylamine (448.23 mg, 4.43 mmol, 617.40 μL) and tert-butyl 2-bromoacetate (345.60 mg, 1.77 mmol, 259.85 μL) at 0° C. The reaction mixture was stirred at room temperature for 4 h. The reaction mixture was concentrated under reduced pressure. The crude compound was purified by reverse phase prep HPLC [Purification method: Column: XBridge C8 (150×19) mm, 5 um; Mobile phase A: 0.1% TFA in water and Mobile phase B: MeCN] to afford tert-butyl 2-(3-(1-(2,6-bis(benzyloxy)pyridin-3-yl)-3-methyl-2-oxo-2,3-dihydro-1H-benzo[d]imidazol-5-yl)pyrrolidin-1-yl)acetate (6, 800 mg, 1.21 mmol, 70% yield, TFA salt) as a pale yellow solid.

The enantiomers of int-6 were separated by chiral SFC:

Method details: Column name—Lux C3; Co solvent—0.5% Isopropyl amine in Methanol 40%, Flow rate: 5 mL/min, Outlet Pressure: 100 bar.

The first eluted fraction at RT 1.29 min: tert-butyl 2-[(3R)-3-[1-(2,6-dibenzyloxy-3-pyridyl)-3-methyl-2-oxo-benzimidazol-5-yl]pyrrolidin-1-yl]acetate (7a, First eluted fraction, 350 mg, 544.06 μmol, 37% yield) (Chiral purity: 99.83%) was isolated as a pale yellow gummy liquid.

LCMS (ES+): m/z 621.2 [M+H]⁺

Second eluted fraction RT 2.14 min: tert-butyl 2-[(3S)-3-[1-(2,6-dibenzyloxy-3-pyridyl)-3-methyl-2-oxo-benzimidazol-5-yl]pyrrolidin-1-yl]acetate (7b, Second eluted fraction, 310 mg, 481.53 μmol, 33% yield) (RT 1.77, optical purity 99.81%) was isolated as a pale yellow gummy liquid.

LCMS (ES+): m/z 621.2 [M+H]⁺

### Step 5: tert-butyl 2-[(3R)-3-[1-(2,6-dioxo-3-piperidyl)-3-methyl-2-oxo-benzimidazol-5-yl]pyrrolidin-1-yl]acetate (8a)

Into a 50 mL single neck round bottom flask containing a well-stirred solution tert-butyl 2-[(3R)-3-[1-(2,6-dibenzyloxy-3-pyridyl)-3-methyl-2-oxo-benzimidazol-5-yl]pyrrolidin-1-yl]acetate (7a, 350 mg, 541.29 μmol) in 1,4-dioxane (7 mL) was added 20 wt. % palladium on carbon (380.10 mg, 541.29 μmol, 20% purity) at room temperature. The reaction mixture was stirred under hydrogen atmosphere (bladder) at room temperature. After 16 h, the reaction mixture was filtered through Celite and washed with 1,4-dioxane (100 mL). The filtrate was concentrated under reduced pressure to obtain crude compound, which was purified by reverse phase column chromatography [Siliasep C18 120 g, Mobile phase A: 0.1% formic acid in water; Mobile phase B: MeCN] to obtain tert-butyl 2-[(3R)-3-[1-(2,6-dioxo-3-piperidyl)-3-methyl-2-oxo-benzimidazol-5-yl]pyrrolidin-1-yl]acetate (8a, 240 mg, 487.29 μmol, 90% yield, formic acid salt) as a pale yellow solid.

LCMS (ES+): m/z 443.2 [M+H]⁺

### Step 6: 2-[(3R)-3-[1-(2,6-dioxo-3-piperidyl)-3-methyl-2-oxo-benzimidazol-5-yl]pyrrolidin-1-yl]acetic acid (9a)

Into a 50 mL single neck round bottom flask containing a well-stirred solution of tert-butyl 2-[(3R)-3-[1-(2,6-dioxo-3-piperidyl)-3-methyl-2-oxo-benzimidazol-5-yl]pyrrolidin-1-yl]acetate (8a, 230 mg, 514.57 μmol) in DCM (4 mL) was added trifluoroacetic acid (880.07 mg, 7.72 mmol, 594.64 μL) at 0° C. The reaction mixture was stirred at room temperature for 2 h. The volatiles were removed under reduced pressure and the residue was triturated with diethyl ether (5 mL), filtered and dried to afford 2-[(3R)-3-[1-(2,6-dioxo-3-piperidyl)-3-methyl-2-oxo-benzimidazol-5-yl]pyrrolidin-1-yl]acetic acid (9a, 210 mg, 417.42 μmol, 81% yield, TFA salt) as an off-white solid.

LCMS (ES+): m/z 387.2 [M+H]⁺

### 2-((3S)-3-(1-(2,6-dioxopiperidin-3-yl)-3-methyl-2-oxo-2,3-dihydro-1H-benzo[d]imidazol-5-yl)pyrrolidin-1-yl)acetic acid (3)

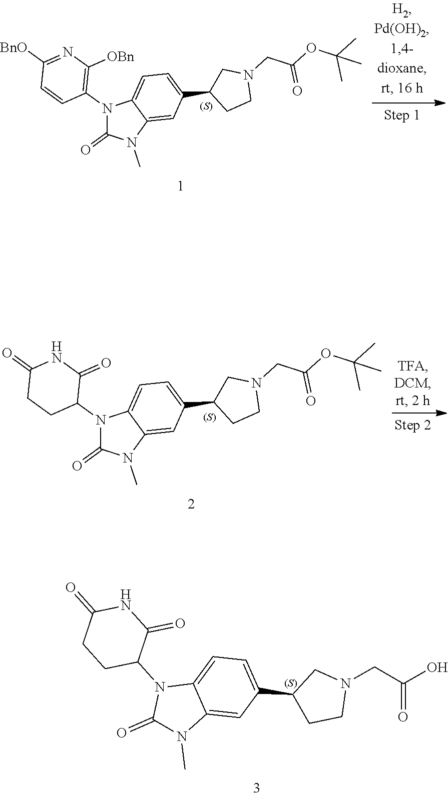

2-((3S)-3-(1-(2,6-dioxopiperidin-3-yl)-3-methyl-2-oxo-2,3-dihydro-1H-benzo[d]imidazol-5-yl)pyrrolidin-1-yl)acetic acid (3, 180 mg, 357.14 μmol, 84% yield, TFA salt) was synthesized following the same two step procedure as 2-[(3R)-3-[1-(2,6-dioxo-3-piperidyl)-3-methyl-2-oxo-benzimidazol-5-yl]pyrrolidin-1-yl]acetic acid.

### 2-[4-[3-(2,4-dioxohexahydropyrimidin-1-yl)-5-fluoro-1-methyl-indazol-6-yl]phenyl]acetic acid (3)

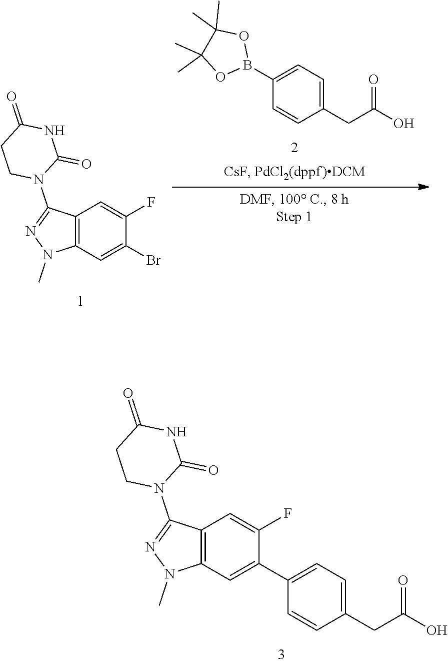 [SMILES: Cn1nc(N2CCC(=O)NC2=O)c2cc(F)c(Br)cc21 ; CC1(C)OB(c2ccc(CC(=O)O)cc2)OC1(C)C ; Cn1nc(N2CCC(=O)NC2=O)c2cc(F)c(-c3ccc(CC(=O)O)cc3)cc21]

### Step 1: 2-[4-[3-(2,4-dioxohexahydropyrimidin-1-yl)-5-fluoro-1-methyl-indazol-6-yl]phenyl]acetic acid (3)

Into a 10 mL sealed-tube containing a well-stirred solution of 1-(6-bromo-5-fluoro-1-methyl-indazol-3-yl)hexahydropyrimidine-2,4-dione (1, 200 mg, 586.28 μmol) and 2-[4-(4,4,5,5-tetramethyl-1,3,2-dioxaborolan-2-yl)phenyl]acetic acid (2, 153.67 mg, 586.28 μmol) in anhydrous DMF (4 mL) was added cesium fluoride (267.17 mg, 1.76 mmol) at room temperature. Nitrogen gas was purged through a reaction mixture for 10 min. Then PdCl₂(dppf)·DCM complex (95.76 mg, 117.26 μmol) was added and stirring was continued for 8 h at 100° C. The reaction was passed through Celite and concentrated under reduced pressure to get the crude compound that was purified by reverse phase column chromatography [Column: Silicycle C18, Mobile phase A: 0.1% FA in water and Mobile Phase B: MeCN] to get 2-[4-[3-(2,4-dioxohexahydropyrimidin-1-yl)-5-fluoro-1-methyl-indazol-6-yl]phenyl]acetic acid (3, 150 mg, 367.04 μmol, 63% yield) as an off-white solid.

LCMS (ES+): m/z 397.2 [M+H]⁺

### 2-[(3R,4S)-1-[1-(2,6-dibenzyloxy-3-pyridyl)-3-methyl-2-oxo-benzimidazol-5-yl]-3-methyl-4-piperidyl]acetic acid (2)

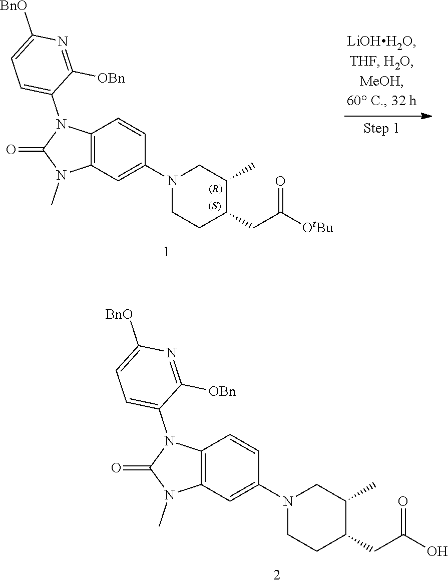

Note: Configurations are arbitrarily assigned.

### Step 1: 2-[(3R,4S)-1-[1-(2,6-dibenzyloxy-3-pyridyl)-3-methyl-2-oxo-benzimidazol-5-yl]-3-methyl-4-piperidyl]acetic acid (2)

Into a 100 mL single-neck round bottom flask containing a well-stirred solution of tert-butyl 2-[(3R,4S)-1-[1-(2,6-dibenzyloxy-3-pyridyl)-3-methyl-2-oxo-benzimidazol-5-yl]-3-methyl-4-piperidyl]acetate (1, 280 mg, 431.57 μmol) in THF (6 mL), water (3 mL) and methanol (6 mL) was added lithium hydroxide, monohydrate (271.63 mg, 6.47 mmol) and the reaction mixture was stirred at 60° C. After 32 h, the reaction mixture was concentrated under reduced pressure and acidified with aq. 1.5 N HCl solution. The solid obtained was filtered and dried to get 2-[(3R,4S)-1-[1-(2,6-dibenzyloxy-3-pyridyl)-3-methyl-2-oxo-benzimidazol-5-yl]-3-methyl-4-piperidyl]acetic acid (2,260 mg, 400.45 μmol, 93% yield, HCl salt) as an off-white solid.

LCMS (ES+): m/z 593.2 [M+H]⁺

### 2-[(3S,4R)-1-[1-(2,6-dibenzyloxy-3-pyridyl)-3-methyl-2-oxo-benzimidazol-5-yl]-3-methyl-4-piperidyl]acetic acid (2)

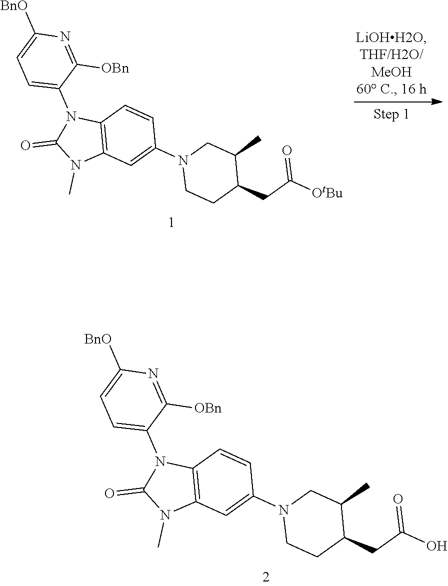 [SMILES: C[C@@H]1CN(c2ccc3c(c2)n(C)c(=O)n3-c2ccc(OCc3ccccc3)nc2OCc2ccccc2)CC[C@@H]1CC(=O)OC(C)(C)C ; C[C@@H]1CN(c2ccc3c(c2)n(C)c(=O)n3-c2ccc(OCc3ccccc3)nc2OCc2ccccc2)CC[C@@H]1CC(=O)O]

2-[(3S,4R)-1-[1-(2,6-dibenzyloxy-3-pyridyl)-3-methyl-2-oxo-benzimidazol-5-yl]-3-methyl-4-piperidyl]acetic acid (2, 225 mg, 335.92 μmol, 78% yield, HCl salt) was synthesized following the same procedure as 2-[(3R,4S)-1-[1-(2,6-dibenzyloxy-3-pyridyl)-3-methyl-2-oxo-benzimidazol-5-yl]-3-methyl-4-piperidyl]acetic acid.

LCMS (ES+): m/z 593.2 [M+H]+

### 2-[4-[4-(2,6-dioxo-3-piperidyl)phenyl]-1-piperidyl]acetic acid (3)

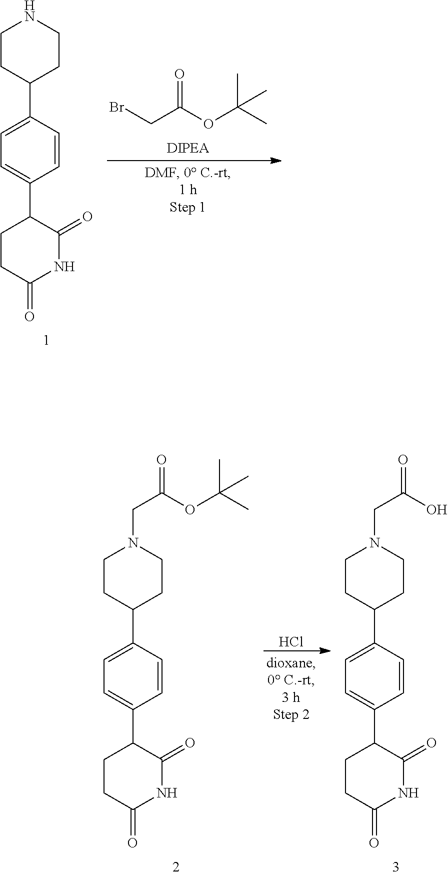

### Step 1: tert-butyl 2-[4-[4-(2,6-dioxo-3-piperidyl)phenyl]-1-piperidyl]acetate (2)

Into a 25 mL round-bottom flask containing a well-stirred solution of 3-[4-(4-piperidyl)phenyl]piperidine-2,6-dione (1, 208.21 mg, 377.59 μmol, HCl salt) in anhydrous DMF (1 mL) at 0° C. was added DIPEA (122.00 mg, 943.97 μmol, 164.42 μL) followed by tert-butyl 2-bromoacetate (88.38 mg, 453.11 μmol, 66.45 μL) under nitrogen atmosphere. The reaction was stirred at room temperature for 1 h, at which point LCMS indicated the completion of the reaction. The reaction mixture was poured into ice-cold water (0.5 mL) to get a precipitate that was filtered and dried under vacuum to afford tert-butyl 2-[4-[4-(2,6-dioxo-3-piperidyl)phenyl]-1-piperidyl]acetate (2, 120 mg, 307.39 μmol, 81% yield) as a white solid.

LCMS (ES+): m/z 387.2 [M+H]⁺

### Step 2: 2-[4-[4-(2,6-dioxo-3-piperidyl)phenyl]-1-piperidyl]acetic acid (3)

Into a 25 mL single neck round-bottom flask containing a well-stirred solution of tert-butyl 2-[4-[4-(2,6-dioxo-3-piperidyl)phenyl]-1-piperidyl]acetate (2, 120 mg, 307.39 μmol) in dry 1,4-dioxane (0.5 mL) was added HCl (4M in 1,4-dioxane, 6.15 mmol, 1.5 mL) at 0° C. The resultant mixture was stirred for 3 h at room temperature. The solvent was evaporated and the residue was washed with MTBE (10 mL) to afford 2-[4-[4-(2,6-dioxo-3-piperidyl)phenyl]-1-piperidyl]acetic acid (3, 95 mg, 253.01 μmol, 82% yield, HCl salt) as white solid.

UPLC-MS (ES+): m/z 331.2 [M+H]⁺

### 1-[6-[4-(2-aminoethyl)-1-piperidyl]-5-fluoro-1-methyl-indazol-3-yl]hexahydropyrimidine-2,4-dione (4)

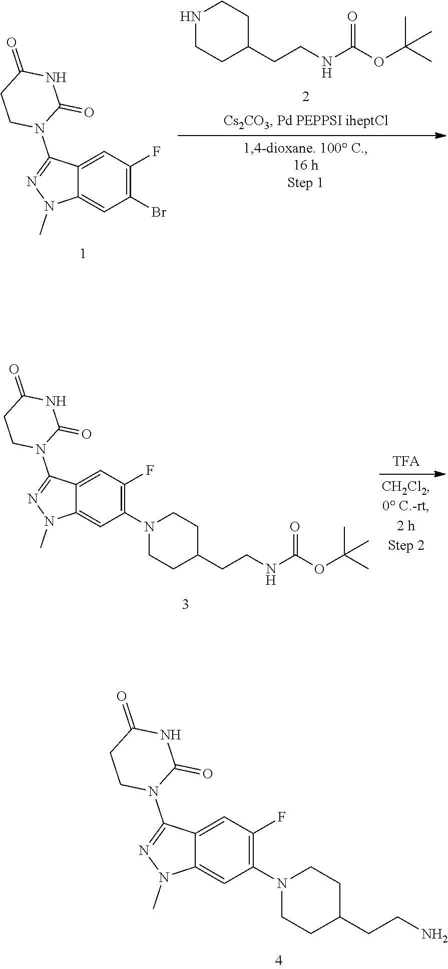 [SMILES: Cn1nc(N2CCC(=O)NC2=O)c2cc(F)c(Br)cc21 ; CC(C)(C)OC(=O)NCCC1CCNCC1 ; Cn1nc(N2CCC(=O)NC2=O)c2cc(F)c(N3CCC(CCNC(=O)OC(C)(C)C)CC3)cc21 ; "TFA" ; Cn1nc(N2CCC(=O)NC2=O)c2cc(F)c(N3CCC(CCN)CC3)cc21]

### Step 1: tert-butyl N-[2-[1-[3-(2,4-dioxohexahydropyrimidin-1-yl)-5-fluoro-1-methyl-indazol-6-yl]-4-piperidyl]ethyl]carbamate (3)

Into a 25 mL sealed-tube containing a well-stirred solution of 1-(6-bromo-5-fluoro-1-methyl-indazol-3-yl)hexahydropyrimidine-2,4-dione (1, 250 mg, 732.85 μmol), tert-butyl N-[2-(4-piperidyl)ethyl]carbamate (2, 334.66 mg, 1.47 mmol) in anhydrous 1,4-dioxane (6 mL) was added cesium carbonate (716.33 mg, 2.20 mmol) at room temperature. Nitrogen gas was purged through the reaction mixture for 10 min. Then Pd-PEPPSI™-IHept C1 3-chloropyridine (35.64 mg, 36.64 μmol) was added and the mixture was heated at 100° C. for 16 h. The reaction mixture water diluted with water (50 mL) and extracted with EtOAc (3×30 mL). The combined organic layers were dried over anhydrous Na₂SO₄, filtered and concentrated under reduced pressure to get the crude material that was purified by reverse-phase [Column: Silicycle C18-120 g; Mobile phase A: 10 mM NH₄HCO₃ in water and Mobile Phase B: MeCN] to get the tert-butyl N-[2-[1-[3-(2,4-dioxohexahydropyrimidin-1-yl)-5-fluoro-1l-methyl-indazol-6-yl]-4-piperidyl]ethyl]carbamate (3, 95 mg, 187.10 μmol, 26% yield).

LCMS (ES+): m/z 489.2 [M+H]⁺

### Step 2: 1-[6-[4-(2-aminoethyl)-1-piperidyl]-5-fluoro-1-methyl-indazol-3-yl]hexahydropyrimidine-2,4-dione (4)

Into a 25 mL single-neck round-bottom flask containing a well-stirred solution of tert-butyl N-[2-[1-[3-(2,4-dioxohexahydropyrimidin-1-yl)-5-fluoro-1-methyl-indazol-6-yl]-4-piperidyl]ethyl]carbamate (3, 80 mg, 157.56 μmol) in anhydrous DCM (2 mL) was added TFA (740.00 mg, 6.49 mmol, 0.5 mL) at 0° C. The resulting mixture was stirred at room temperature for 2 h. The solvent was evaporated and the residue was washed with MTBE to get 1-[6-[4-(2-aminoethyl)-1-piperidyl]-5-fluoro-1-methyl-indazol-3-yl]hexahydropyrimidine-2,4-dione (4, 75 mg, 145.28 μmol, 92% yield, TFA salt) as a white solid.

LCMS (ES+): m/z 389.2 [M+H]⁺

### 2-[(3R,4R)-4-[1-(2,6-dioxo-3-piperidyl)-3-ethyl-2-oxo-benzimidazol-5-yl]-3-fluoro-1-piperidyl]acetic acid (6)

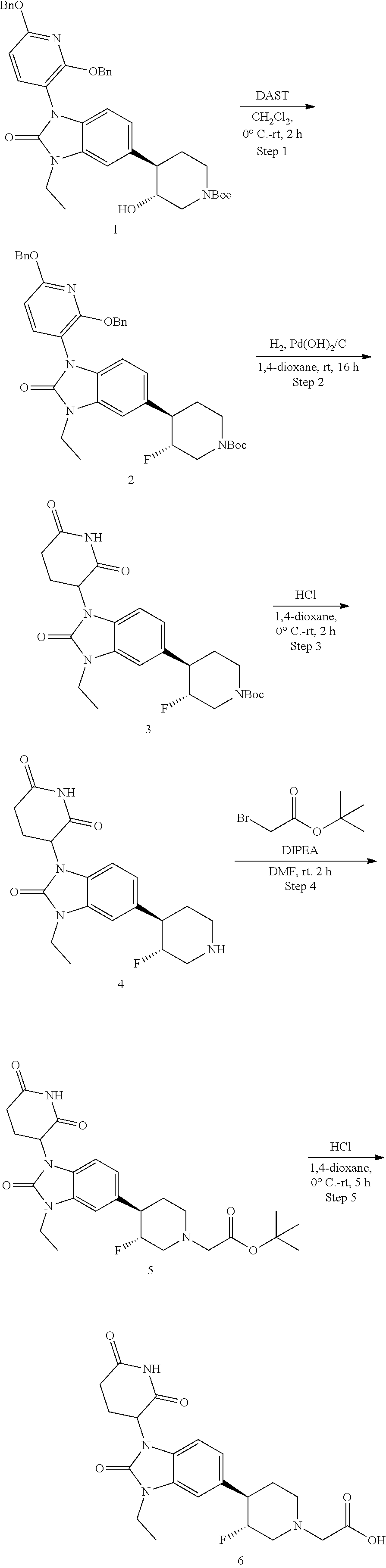

Note: Configurations are arbitrarily assigned.

### Step 1: tert-butyl (3R,4R)-4-[1-(2,6-dioxo-3-piperidyl)-3-ethyl-2-oxo-benzimidazol-5-yl]-3-fluoro-piperidine-1-carboxylate_(2)

Into a 100 mL single-neck round-bottom flask containing a well-stirred solution of tert-butyl (3R,4R)-4-[1-(2,6-dibenzyloxy-3-pyridyl)-3-ethyl-2-oxo-benzimidazol-5-yl]-3-hydroxy-piperidine-1-carboxylate (1, 750 mg, 1.14 mmol) in anhydrous DCM (40 mL) was added DAST (551.74 mg, 3.42 mmol, 452.25 μL) at 0° C. and the resulting mixture was stirred for 2 h at room temperature. The reaction mixture was cooled to 0° C. and quenched with saturated NaHCO₃ solution (30 mL) and extracted with DCM (2×200 mL). The combined organic layers were washed with brine solution (20 mL), dried with Na₂SO₄, filtered and concentrated under reduced pressure to get the crude material that was purified by silica gel flash column chromatography (230-400 mesh silica gel; 30% EtOAc in Pet ether) to afford tert-butyl (3R,4R)-4-[1-(2,6-dibenzyloxy-3-pyridyl)-3-ethyl-2-oxo-benzimidazol-5-yl]-3-fluoro-piperidine-1-carboxylate (2, 260 mg, 385.97 μmol, 34% yield) LCMS (ES+): m/z 653.2 [M+H]⁺

### Step 2: tert-butyl (3R,4R)-4-[1-(2,6-dioxo-3-piperidyl)-3-ethyl-2-oxo-benzimidazol-5-yl]-3-fluoro-piperidine-1-carboxylate (3)

Into a 50 mL single-neck round-bottom flask containing a well-stirred solution of tert-butyl (3R,4R)-4-[1-(2,6-dibenzyloxy-3-pyridyl)-3-ethyl-2-oxo-benzimidazol-5-yl]-3-fluoro-piperidine-1-carboxylate (2, 260 mg, 382.38 μmol) in anhydrous 1,4-dioxane (20 mL) was added palladium hydroxide on carbon (20 wt. % 50% water, 260 mg, 1.85 mmol) at room temperature. The mixture was stirred at room temperature for 16 h under a hydrogen gas bladder. The reaction mixture was passed through Celite and washed with 1,4-dioxane (100 mL). The filtrate was concentrated under reduced pressure to afford tert-butyl (3R,4R)-4-[1-(2,6-dioxo-3-piperidyl)-3-ethyl-2-oxo-benzimidazol-5-yl]-3-fluoro-piperidine-1-carboxylate (3, 180 mg, 379.02 μmol, 99% yield) as brown gummy solid.

LCMS (ES−): m/z 473.1 [M−H]⁻

### Step 3: 3-[3-ethyl-5-[(3R,4R)-3-fluoro-4-piperidyl]-2-oxo-benzimidazol-1-yl]piperidine-2,6-dione (4)

Into a 50 mL single-neck round-bottom flask containing a well-stirred solution of tert-butyl (3R,4R)-4-[1-(2,6-dioxo-3-piperidyl)-3-ethyl-2-oxo-benzimidazol-5-yl]-3-fluoro-piperidine-1-carboxylate (3, 310 mg, 587.96 μmol) in dry 1,4-dioxane (1 mL) was added HCl (4.0 M in 1,4-dioxane, 11.76 mmol, 3.0 mL) at 0° C. The resultant reaction mixture was stirred for 2 h at room temperature. The solvent was evaporated and the residue was triturated with MTBE (10 mL) to afford 3-[3-ethyl-5-[(3R,4R)-3-fluoro-4-piperidyl]-2-oxo-benzimidazol-1-yl]piperidine-2,6-dione (4, 250 mg, 474.60 μmol, 81% yield, HCl salt) as an off-white solid.

LCMS (ES+): m/z 375.2 [M+H]⁺

### Step 4: tert-butyl 2-[(3R,4R)-4-[1-(2,6-dioxo-3-piperidyl)-3-ethyl-2-oxo-benzimidazol-5-yl]-3-fluoro-1-piperidyl]acetate (5)

Into a 50 mL single-neck round-bottom flask containing a well-stirred solution of 3-[3-ethyl-5-[(3R,4R)-3-fluoro-4-piperidyl]-2-oxo-benzimidazol-1-yl]piperidine-2,6-dione (4, 250 mg, 474.60 μmol, HCl salt) in anhydrous DMF (3 mL) at 0° C. under nitrogen atmosphere were added tert-butyl bromoacetate (111.09 mg, 569.52 μmol, 83.52 μL) followed by DIPEA (184.01 mg, 1.42 mmol, 248.00 uL). The resulting reaction mixture was stirred at room temperature for 2 h. The reaction mixture was diluted with water (5 mL) and extracted with DCM (2×100 mL).

The combined organic layers were dried over Na₂SO₄, and concentrated under reduced pressure to afford tert-butyl 2-[(3R,4R)-4-[1-(2,6-dioxo-3-piperidyl)-3-ethyl-2-oxo-benzimidazol-5-yl]-3-fluoro-1-piperidyl]acetate (5, 240 mg, 445.68 μmol, 94% yield) as an off-white solid.

LCMS (ES+): m/z 489.2 [M+H]⁺

### Step 5: 2-[(3R,4R)-4-[1-(2,6-dioxo-3-piperidyl)-3-ethyl-2-oxo-benzimidazol-5-yl]-3-fluoro-1-piperidyl]acetic acid (6

Into a 50 mL single-neck round-bottom flask containing a well-stirred solution of tert-butyl 2-[(3R,4R)-4-[1-(2,6-dioxo-3-piperidyl)-3-ethyl-2-oxo-benzimidazol-5-yl]-3-fluoro-1-piperidyl]acetate (5, 220 mg, 387.27 μmol) in dry 1,4-dioxane (1 mL) was added HCl (4.0 M in 1,4-dioxane, 8.13 mmol, 2 mL) at 0° C. The resultant reaction mixture was stirred for 5 h at room temperature. The solvent was evaporated and the resultant residue was washed with MTBE (5 mL) to afford 2-[(3R,4R)-4-[1-(2,6-dioxo-3-piperidyl)-3-ethyl-2-oxo-benzimidazol-5-yl]-3-fluoro-1-piperidyl]acetic acid (6, 180 mg, 318.23 μmol, 82% yield, HCl salt) as a brown gummy solid.

LCMS (ES+): m/z 433.2 [M+H]⁺

### 2-[(3S,4S)-4-[1-(2,6-dioxo-3-piperidyl)-3-ethyl-2-oxo-benzimidazol-5-yl]-3-fluoro-1-piperidyl]acetic acid (6)

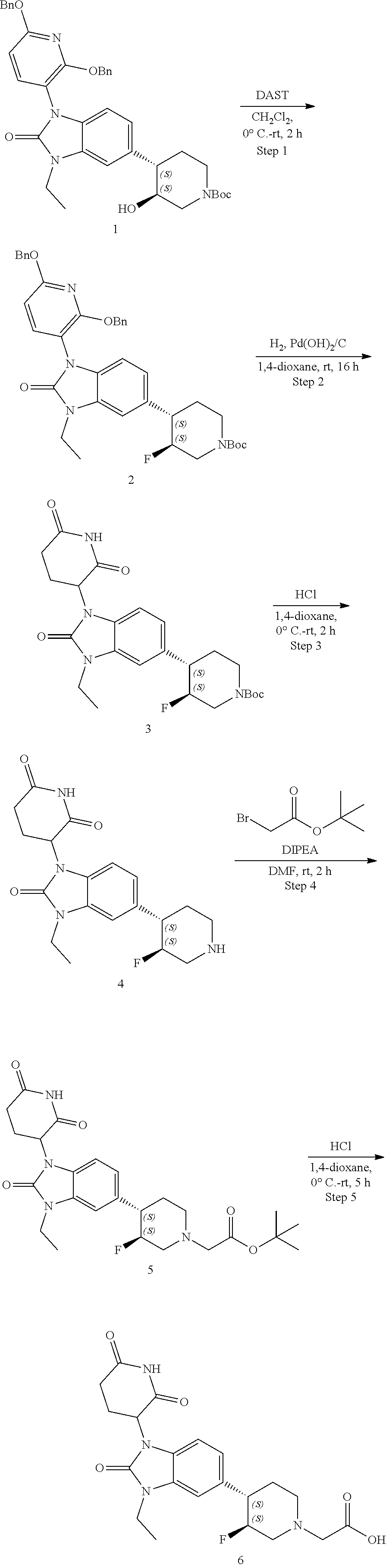

Note: Configurations are arbitrarily assigned.

### Step 1: tert-butyl (3S,4S)-4-[1-(2,6-dioxo-3-piperidyl)-3-ethyl-2-oxo-benzimidazol-5-yl]-3-fluoro-piperidine-1-carboxylate (2)

Into a 100 mL single-neck round-bottom flask containing a well-stirred solution of tert-butyl (3S,4S)-4-[1-(2,6-dibenzyloxy-3-pyridyl)-3-ethyl-2-oxo-benzimidazol-5-yl]-3-hydroxy-piperidine-1-carboxylate (1, 1.0 g, 1.51 mmol) in anhydrous DCM (40 mL) was added DAST (485.48 mg, 3.01 mmol) at 0° C. and the resulting mixture was stirred for 2 h at room temperature. The reaction mixture was cooled to 0° C. and quenched with saturated NaHCO₃ solution (50 mL) and extracted with DCM (2×300 mL). The combined organic layers were washed with brine solution (40 mL), dried with Na₂SO₄, filtered and concentrated under reduced pressure to get the crude material that was purified by silica gel flash column chromatography (230-400 mesh silica gel; 30% EtOAc in Pet ether) to afford tert-butyl (3S,4S)-4-[1-(2,6-dibenzyloxy-3-pyridyl)-3-ethyl-2-oxo-benzimidazol-5-yl]-3-fluoro-piperidine-1-carboxylate (2, 500 mg, 739.94 μmol, 49% yield) LCMS (ES+): m/z 653.2 [M+H]⁺

### Step 2: tert-butyl (3S,4S)-4-[1-(2,6-dioxo-3-piperidyl)-3-ethyl-2-oxo-benzimidazol-5-yl]-3-fluoro-piperidine-1-carboxylate (3)

Into a 50 mL single-neck round-bottom flask containing a well-stirred solution of tert-butyl (3S,4S)-4-[1-(2,6-dibenzyloxy-3-pyridyl)-3-ethyl-2-oxo-benzimidazol-5-yl]-3-fluoro-piperidine-1-carboxylate (2, 500 mg, 735.35 μmol,) in anhydrous 1,4-dioxane (40 mL) was added palladium hydroxide on carbon (20 wt. % 50% water, 600 mg, 4.27 mmol) at room temperature. The mixture was stirred at room temperature for 16 h under a hydrogen gas bladder. The reaction mixture was passed through Celite and washed with 1,4-dioxane (100 mL). The filtrate was concentrated under reduced pressure to afford tert-butyl (3S,4S)-4-[1-(2,6-dioxo-3-piperidyl)-3-ethyl-2-oxo-benzimidazol-5-yl]-3-fluoro-piperidine-1-carboxylate (3, 340 mg, 715.15 μmol, 97% yield) as brown gummy solid.

LCMS (ES−): m/z 473.1 [M−H]⁻

### Step 3: 3-[3-ethyl-5-[(3S,4S)-3-fluoro-4-piperidyl]-2-oxo-benzimidazol-1-yl]piperidine-2,6-dione (4)

Into a 50 mL single-neck round-bottom flask containing a well-stirred solution of tert-butyl (3S,4S)-4-[1-(2,6-dioxo-3-piperidyl)-3-ethyl-2-oxo-benzimidazol-5-yl]-3-fluoro-piperidine-1-carboxylate (3, 320 mg, 606.92 μmol,) in dry 1,4-dioxane (2 mL) was added HCl (4.0 M in 1,4-dioxane, 12.14 mmol, 3 mL) at 0° C. The resultant reaction mixture was stirred for 2 h at room temperature. The solvent was evaporated and the residue was triturated with MTBE (10 mL) to afford 3-[3-ethyl-5-[(3S,4S)-3-fluoro-4-piperidyl]-2-oxo-benzimidazol-1-yl]piperidine-2,6-dione (4, 260 mg, 533.45 μmol, 88% yield, HCl salt) as an off-white solid.

LCMS (ES+): m/z 375.2 [M+H]⁺

### Step 4: tert-butyl 2-[(3S,4S)-4-[1-(2,6-dioxo-3-piperidyl)-3-ethyl-2-oxo-benzimidazol-5-yl]-3-fluoro-1-piperidyl]acetate (5)

Into a 50 mL single-neck round-bottom flask containing a well-stirred solution of 3-[3-ethyl-5-[(3S,4S)-3-fluoro-4-piperidyl]-2-oxo-benzimidazol-1-yl]piperidine-2,6-dione (4, 260 mg, 531.56 μmol, HCl salt) in anhydrous DMF (3 mL) at 0° C. under nitrogen atmosphere were added tert-butyl bromoacetate (124.42 mg, 637.87 μmol, 93.55 μL) followed by DIPEA (206.10 mg, 1.59 mmol, 277.76 uL). The resulting reaction mixture was stirred at room temperature for 2 h. The reaction mixture was diluted with water (5 mL) and extracted with DCM (2×100 mL). The combined organic layers were dried over Na₂SO₄, and concentrated under reduced pressure to afford tert-butyl 2-[(3S,4S)-4-[1-(2,6-dioxo-3-piperidyl)-3-ethyl-2-oxo-benzimidazol-5-yl]-3-fluoro-1-piperidyl]acetate (5, 250 mg, 463.50 μmol, 87% yield) as an off-white solid.

LCMS (ES+): m/z 489.2 [M+H]⁺

### Step 5: 2-[(3S,4S)-4-[1-(2,6-dioxo-3-piperidyl)-3-ethyl-2-oxo-benzimidazol-5-yl]-3-fluoro-1-piperidyl]acetic acid (6)

Into a 50 mL single-neck round-bottom flask containing a well-stirred solution of tert-butyl 2-[(3S,4S)-4-[1-(2,6-dioxo-3-piperidyl)-3-ethyl-2-oxo-benzimidazol-5-yl]-3-fluoro-1-piperidyl]acetate (5, 230 mg, 436.88 μmol,) in dry 1,4-dioxane (1 mL) was added HCl (4.0 M in 1,4-dioxane, 13.11 mmol, 3.25 mL) at 0° C. The resultant reaction mixture was stirred for 5 h at room temperature. The solvent was evaporated and the resultant residue was washed with MTBE (5 mL) to afford 2-[(3S,4S)-4-[1-(2,6-dioxo-3-piperidyl)-3-ethyl-2-oxo-benzimidazol-5-yl]-3-fluoro-1-piperidyl]acetic acid (6, 210 mg, 407.54 μmol, 93% yield, HCl salt) as a brown gummy solid.

LCMS (ES+): m/z 433.6 [M+H]⁺

### 2-[(3S,4S)-1-[1-(2,6-dibenzyloxy-3-pyridyl)-3-methyl-2-oxo-benzimidazol-5-yl]-3-methyl-4-piperidyl]acetic acid (2)

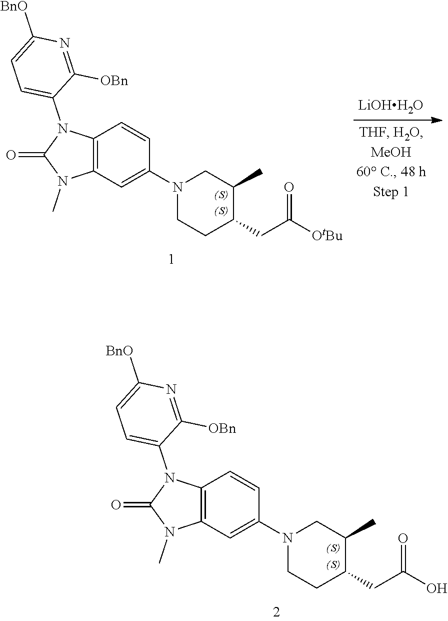

Note: Configurations are arbitrarily assigned.

### Step 1: 2-[(3S,4S)-1-[1-(2,6-dibenzyloxy-3-pyridyl)-3-methyl-2-oxo-benzimidazol-5-yl]-3-methyl-4-piperidyl]acetic acid (2)

Into a 50 mL single-neck round-bottom flask containing a well-stirred solution of tert-butyl 2-[(3S,4S)-1-[1-(2,6-dibenzyloxy-3-pyridyl)-3-methyl-2-oxo-benzimidazol-5-yl]-3-methyl-4-piperidyl]acetate (1, 200.00 mg, 308.27 μmol) in THF (14 mL) and water (2 mL) were added Lithium hydroxide monohydrate (323.37 mg, 7.71 mmol) at room temperature. The reaction mixture was stirred for 48 h at 60° C. The reaction mixture was concentrated under reduced pressure, acidified with aq. 1.5 N HCl and extracted with EtOAc (2×70 mL). The combined organic layers were dried over sodium sulfate and concentrated under vacuum to get 2-[(3S,4S)-1-[1-(2,6-dibenzyloxy-3-pyridyl)-3-methyl-2-oxo-benzimidazol-5-yl]-3-methyl-4-piperidyl]acetic acid (2, 180 mg, 276.19 μmol, 90% yield) as a brown solid.

LCMS (ES+): m/z 593.2 [M+H]⁺

### 2-[(3S,4S)-1-[1-(2,6-dibenzyloxy-3-pyridyl)-3-methyl-2-oxo-benzimidazol-5-yl]-3-methyl-4-piperidyl]acetic acid (2)

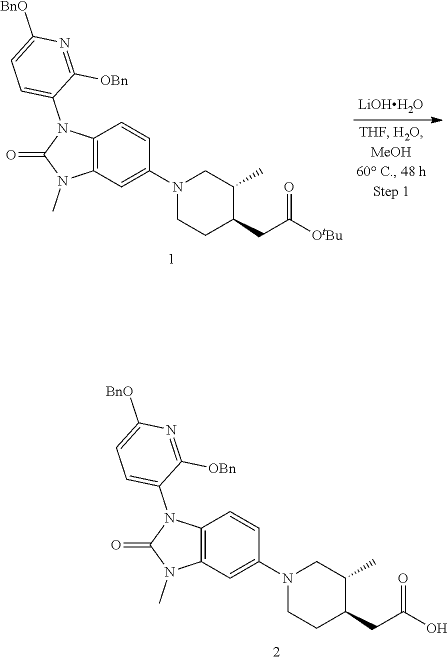 [SMILES: C[C@H]1CN(c2ccc3c(c2)n(C)c(=O)n3-c2ccc(OCc3ccccc3)nc2OCc2ccccc2)CC[C@@H]1CC(=O)OC(C)(C)C ; "LiOH·H2O" ; C[C@H]1CN(c2ccc3c(c2)n(C)c(=O)n3-c2ccc(OCc3ccccc3)nc2OCc2ccccc2)CC[C@@H]1CC(=O)O]

Note: Configurations are arbitrarily assigned.

### Step 1: 2-[(3S,4S)-1-[1-(2,6-dibenzyloxy-3-pyridyl)-3-methyl-2-oxo-benzimidazol-5-yl]-3-methyl-4-piperidyl]acetic acid (2)

Into a 25 mL round-bottom flask containing a well-stirred solution of tert-butyl 2-[(3R,4R)-1-[1-(2,6-dibenzyloxy-3-pyridyl)-3-methyl-2-oxo-benzimidazol-5-yl]-3-methyl-4-piperidyl]acetate (2, 250 mg, 385.33 μmol) in a mixture of THF (14 mL), methanol (4 mL) and water (2 mL) was added lithium hydroxide monohydrate (485.10 mg, 11.56 mmol) at room temperature and the resulting mixture was heated at 60° C. for 72 h. The reaction mixture was carefully acidified by adding 1N HCl and extracted with EtOAc (2×50 mL). The combined organic layers were dried over sodium sulfate and concentrated under reduced pressure to get 2-[(3R,4R)-1-[1-(2,6-dibenzyloxy-3-pyridyl)-3-methyl-2-oxo-benzimidazol-5-yl]-3-methyl-4-piperidyl]acetic acid (2, 240 mg, 377.66 μmol, 98% yield, HCl salt) as an off-white solid.

UPLC-MS (ES+): m/z 593.2 [M+H]⁺

### 2-[1-[3-(2,4-dioxohexahydropyrimidin-1-yl)-5-fluoro-1-methyl-indazol-6-yl]-4-piperidyl]acetic acid (4)

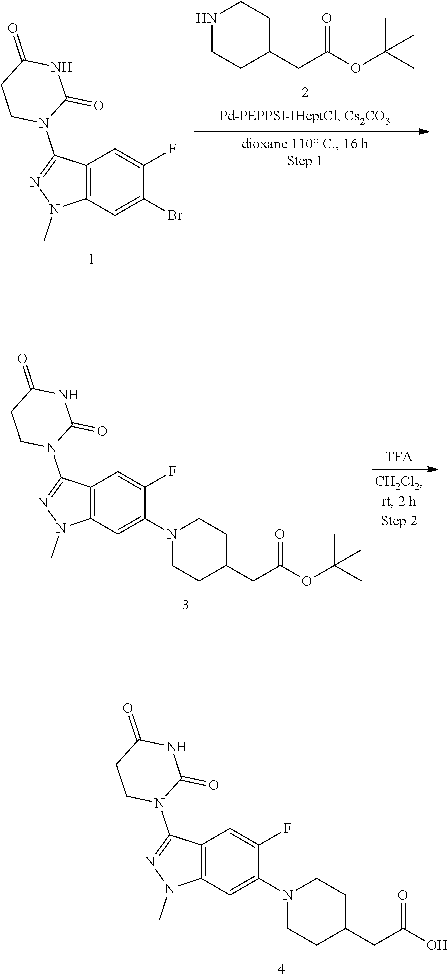 [SMILES: Cn1nc(N2CCC(=O)NC2=O)c2cc(F)c(Br)cc21 ; CC(C)(C)OC(=O)CC1CCNCC1 ; Cn1nc(N2CCC(=O)NC2=O)c2cc(F)c(N3CCC(CC(=O)OC(C)(C)C)CC3)cc21 ; "TFA" ; Cn1nc(N2CCC(=O)NC2=O)c2cc(F)c(N3CCC(CC(=O)O)CC3)cc21]

### Step 1: Preparation of tert-butyl 2-[1-[3-(2,4-dioxohexahydropyrimidin-1-yl)-5-fluoro-1-methyl-indazol-6-yl]-4-piperidyl]acetate (3)

Into a 50 mL sealed-tube containing a well-stirred solution of 1-(6-bromo-5-fluoro-1-methyl-indazol-3-yl)hexahydropyrimidine-2,4-dione (1, 250 mg, 732.85 μmol), tert-butyl 2-(4-piperidyl)acetate (2, 146.05 mg, 732.85 μmol) in anhydrous 1,4-dioxane (5 mL) were added cesium carbonate (716.33 mg, 2.20 mmol) at room temperature. Nitrogen gas was purged through the reaction mixture for 10 min. Then Pd-PEPPSI-IHeptCl (35.64 mg, 36.64 μmol) was added and the mixture was heated at 110° C. for 16 h. The solvent was evaporated from the reaction mixture to get the crude compound that was purified by silica gel column chromatography (100-200 mesh silica gel; 4% EtOAc in Pet ether) to obtain tert-butyl 2-[1-[3-(2,4-dioxohexahydropyrimidin-1-yl)-5-fluoro-1-methyl-indazol-6-yl]-4-piperidyl]acetate (3, 280 mg, 428.98 μmol, 59% yield) as a brown solid.

LCMS (ES+): m/z 460.2 [M+H]⁺

### Step 2: 2-[1-[3-(2,4-dioxohexahydropyrimidin-1-yl)-5-fluoro-1-methyl-indazol-6-yl]-4-piperidyl]acetic acid (4)

Into a 25 mL single-neck round-bottom flask containing a ell-stirred solution of tert-butyl 2-[1-[3-(2,4-dioxohexahydropyrimidin-1-yl)-5-fluoro-1-methyl-indazol-6-yl]-4-piperidyl]acetate (3, 280 mg, 426.54 μmol) in DCM (2 mL) at 0° C., was added TFA (486.35 mg, 4.27 mmol, 328.62 μL) and the resultant mixture was stirred at room temperature for 2 h. The solvent was evaporated to get the crude compound that was purified by reverse-phase preparative HPLC [Column: X-Bridge C8 (150×19) mm, 5 in; Mobile phase A: 0.1% TFA in water and Mobile Phase B: MeCN] to obtain 2-[1-[3-(2,4-dioxohexahydropyrimidin-1-yl)-5-fluoro-1-methyl-indazol-6-yl]-4-piperidyl]acetic acid (4, 220 mg, 415.70 μmol, 97% yield, TFA salt) as a yellow gum.

LCMS (ES+): m/z 404.2 [M+H]⁺

### 2-[4-[[1-(2,6-dioxo-3-piperidyl)-3-methyl-2-oxo-benzimidazol-5-yl]methyl]phenyl]acetic acid (6)

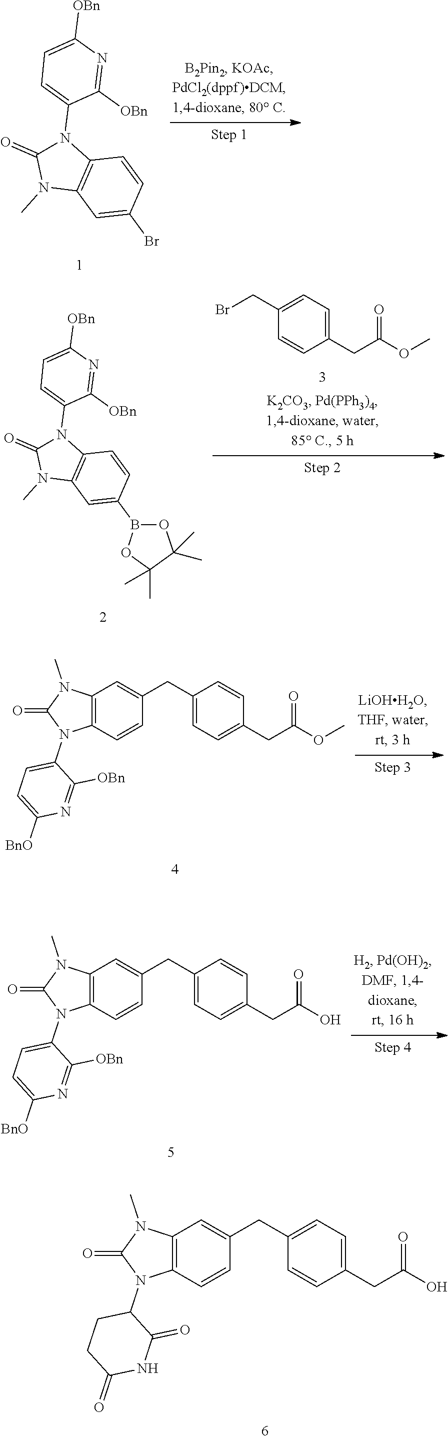 [SMILES: Cn1c(=O)n(-c2ccc(OCc3ccccc3)nc2OCc2ccccc2)c2ccc(Br)cc21 ; Cn1c(=O)n(-c2ccc(OCc3ccccc3)nc2OCc2ccccc2)c2ccc(B3OC(C)(C)C(C)(C)O3)cc21 ; COC(=O)Cc1ccc(CBr)cc1 ; COC(=O)Cc1ccc(Cc2ccc3c(c2)n(C)c(=O)n3-c2ccc(OCc3ccccc3)nc2OCc2ccccc2)cc1 ; Cn1c(=O)n(-c2ccc(OCc3ccccc3)nc2OCc2ccccc2)c2ccc(Cc3ccc(CC(=O)O)cc3)cc21 ; Cn1c(=O)n(C2CCC(=O)NC2=O)c2ccc(Cc3ccc(CC(=O)O)cc3)cc21]

### Step 1:1-(2,6-dibenzyloxy-3-pyridyl)-3-methyl-5-(4,4,5,5-tetramethyl-1,3,2-dioxaborolan-2-yl)benzimidazol-2-one (2)

Into a 50 mL pressure tube containing a well-stirred solution of 5-bromo-1-(2,6-dibenzyloxy-3-pyridyl)-3-methyl-benzimidazol-2-one (1, 1 g, 1.88 mmol) in anhydrous 1,4-dioxane (8 mL) was added potassium acetate (553.06 mg, 5.64 mmol), bis(pinacolato)diboron (1.43 g, 5.64 mmol) and Pd(dppf)Cl₂·DCM (153.40 mg, 187.84 μmol) at ambient temperature under nitrogen atmosphere. The resulting mixture was degassed by bubbling nitrogen gas into the reaction mixture for 10 min. The reaction mixture was heated to 100° C. After 16 h, the reaction mixture was filtered through Celite bed and washed with ethyl acetate (500 mL). The filtrate layer was washed with water (300 mL) and brine (200 mL), dried over anhydrous Na₂SO₄ and filtered. The filtrate was concentrated under reduced pressure to get a crude product which was purified by flash silica-gel column chromatography (0-35% Ethyl acetate in pet ether) to afford 1-(2,6-dibenzyloxy-3-pyridyl)-3-methyl-5-(4,4,5,5-tetramethyl-1,3,2-dioxaborolan-2-yl)benzimidazol-2-one (2, 1 g, 1.67 mmol, 89% yield) as an off-white semisolid.

LCMS (ES+): m/z 564.2 [M+H]⁺

### Step 2: methyl 2-[4-[[1-(2,6-dibenzyloxy-3-pyridyl)-3-methyl-2-oxo-benzimidazol-5-yl]methyl]phenyl]acetate (4)

Into a 50 mL pressure tube containing a well-stirred solution of 1-(2,6-dibenzyloxy-3-pyridyl)-3-methyl-5-(4,4,5,5-tetramethyl-1,3,2-dioxaborolan-2-yl)benzimidazol-2-one (2, 1 g, 1.67 mmol) and methyl 2-[4-(bromomethyl)phenyl]acetate (3, 1.62 g, 6.67 mmol) in 1,4-dioxane (6 mL) and water (1.2 mL) were added potassium carbonate (691.70 mg, 5.00 mmol) and bis(triphenylphosphine)palladium(II) dichloride (234.20 mg, 333.66 μmol). The reaction mixture was degassed by bubbling nitrogen gas for 10 min and heated at 90° C. After 16 h, the reaction mixture was filtered and washed thoroughly with ethyl acetate (200 mL). The filtrate was concentrated under reduced pressure. The crude compound was purified by flash silica gel column chromatography (20-60% ethyl acetate in pet ether) to afford methyl 2-[4-[[1-(2,6-dibenzyloxy-3-pyridyl)-3-methyl-2-oxo-benzimidazol-5-yl]methyl]phenyl]acetate (4, 370 mg, 524.45 μmol, 31% yield) as a brown color liquid.

UP-LCMS (ES+): m/z 600.3 [M+H]⁺

### Step 3: 2-[4-[[1-(2,6-dibenzyloxy-3-pyridyl)-3-methyl-2-oxo-benzimidazol-5-yl]methyl]phenyl]acetic acid (5)

Into a 50 mL round bottom flask containing a well-stirred solution of methyl 2-[4-[[1-(2,6-dibenzyloxy-3-pyridyl)-3-methyl-2-oxo-benzimidazol-5-yl]methyl]phenyl]acetate (4, 370 mg, 524.45 μmol) in THF (2.5 mL) and water (2.5 mL) was added lithium hydroxide monohydrate (154.06 mg, 3.67 mmol) and the reaction mixture was stirred at room temperature. After 4 h, the reaction mixture was concentrated under vacuum and suspended in 1.5 N HCl. The aqueous layer was extracted with ethyl acetate (2×100 mL). The combined organic layers were washed with water (100 mL) followed by brine solution (100 mL), dried over anhydrous sodium sulfate and filtered. The filtrate was concentrated under vacuum to get 2-[4-[[1-(2,6-dibenzyloxy-3-pyridyl)-3-methyl-2-oxo-benzimidazol-5-yl]methyl]phenyl]acetic acid (5, 330 mg, 478.73 μmol, 91% yield) as a brown solid.

LCMS (ES+): m/z 586.2 [M+H]⁺

### Step 4: 2-[4-[[1-(2,6-dioxo-3-piperidyl)-3-methyl-2-oxo-benzimidazol-5-yl]methyl]phenyl]acetic acid (6)

Into a 50 mL single-neck round-bottom flask containing a well-stirred solution of 2-[4-[[1-(2,6-dibenzyloxy-3-pyridyl)-3-methyl-2-oxo-benzimidazol-5-yl]methyl]phenyl]acetic acid (5, 330 mg, 473.32 μmol) in 1,4-dioxane (2.5 mL) and DMF (0.5 mL) was added 20 wt. % palladium hydroxide on carbon (332.35 mg, 473.32 μmol, 20% purity). The reaction mixture was stirred at room temperature under hydrogen atmosphere for 16 h. After completion of the reaction, the reaction mixture was filtered through Celite and washed with 1:1 mixture of 1,4-dioxane and DMF (50 mL). The filtrate was concentrated under reduced pressure to get 2-[4-[[1-(2,6-dioxo-3-piperidyl)-3-methyl-2-oxo-benzimidazol-5-yl]methyl]phenyl]acetic acid (6, 180 mg, 437.05 μmol, 92% yield) as an off-white solid.

LCMS (ES+): m/z 408.2 [M+H]⁺

### tert-butyl 4-[[1-(2,6-dioxo-3-piperidyl)-3-methyl-2-oxo-benzimidazol-5-yl]methyl]piperazine-1-carboxylate (3)

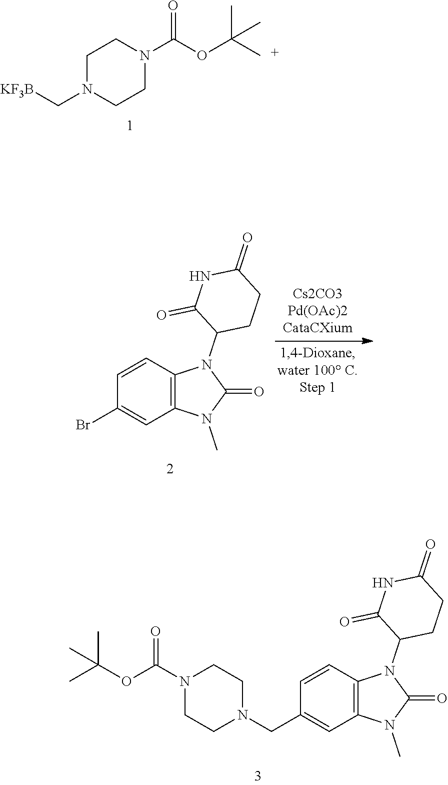

### Step 1: tert-butyl 4-[[1-(2,6-dioxo-3-piperidyl)-3-methyl-2-oxo-benzimidazol-5-yl]methyl]piperazine-1-carboxylate (3)

Into a 25 mL sealed tube containing a well-stirred solution of potassium ((4-(tert-butoxycarbonyl)piperazin-1-yl)methyl)trifluoroborate (1, 113.18 mg, 369.65 μmol) and 3-(5-bromo-3-methyl-2-oxo-benzimidazol-1-yl)piperidine-2,6-dione (2, 50 mg, 147.86 μmol) in 1,4-Dioxane (5 mL) and Water (0.5 mL) was added cesium carbonate (144.53 mg, 443.58 μmol) under nitrogen atmosphere. The reaction mixture was degassed with nitrogen for 5 min. Palladium (II) acetate (3.32 mg, 14.79 μmol) and CataCXium (1.78 mg, 4.95 μmol) were added and the reaction mixture was heated to 100° C. for 5 h. The reaction mixture was cooled to ambient temperature, filtered through Celite and washed with EtOAc. The filtrate was concentrated under reduced pressure to and triturated with MTBE (10 mL). The material was purified via reverse phase preparatory HPLC (Column: X-SELECT C18 (250*19) MM 5 MICRON mobile phase: A: 0.1% TFA in water, B: ACN] to afford tert-butyl 4-[[1-(2,6-dioxo-3-piperidyl)-3-methyl-2-oxo-benzimidazol-5-yl]methyl]piperazine-1-carboxylate (3, 55 mg, 95.83 μmol, 65% yield, TFA salt) as an off white solid.

LCMS (ES+): m/z 458.2 [M+H]⁺

### tert-butyl 3-[1-[1-(2,6-dioxo-3-piperidyl)-3-methyl-2-oxo-benzimidazol-5-yl]-4-piperidyl]propanoate (4)

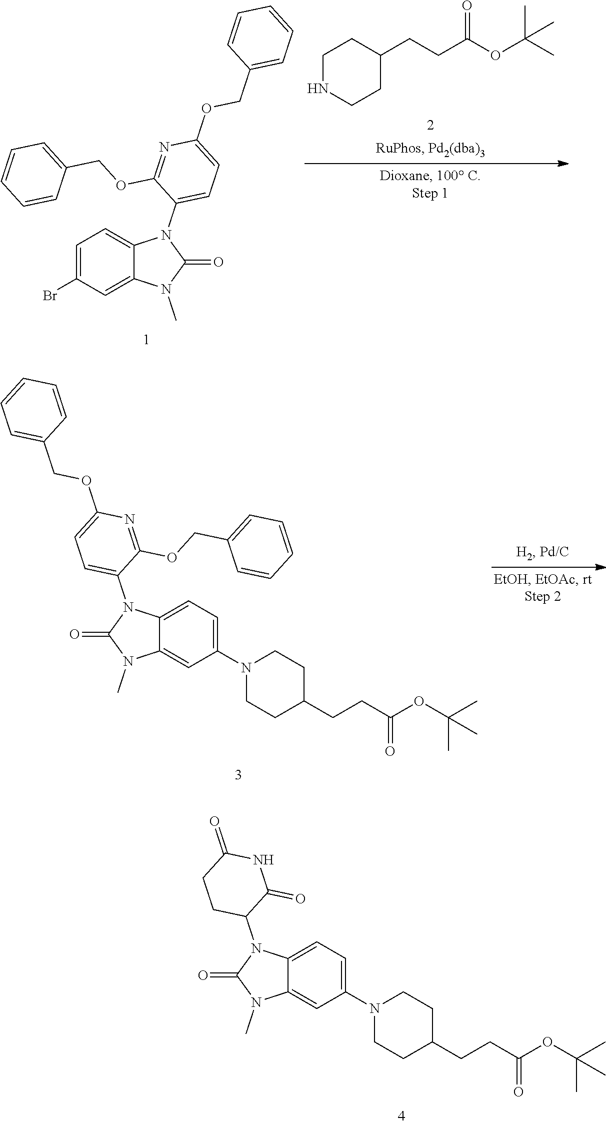

### Step 1: tert-butyl 3-[1-[1-(2,6-dibenzyloxy-3-pyridyl)-3-methyl-2-oxo-benzimidazol-5-yl]-4-piperidyl]propanoate (3)

In a microwave vial, a stirred solution of 5-bromo-1-(2,6-dibenzyloxy-3-pyridyl)-3-methyl-benzimidazol-2-one (1, 1.8 g, 3.49 mmol), tert-butyl 3-(4-piperidyl)propanoate (2, 1.12 g, 5.23 mmol), sodium tert-butoxide (1.34 g, 13.94 mmol), RuPhos (162.66 mg, 348.58 μmol) in Dioxane (25 mL) was degassed with nitrogen for 5 mins. (1E,4E)-1,5-diphenylpenta-1,4-dien-3-one; palladium (319.20 mg, 348.58 μmol) was added and the mixture degassed for 5 mins. After 1 h at 100° C. under microwave irradiation, the reaction mixture was concentrated. Water was added and extracted with ethyl acetate. The organic layer was separated, dried over sodium sulfate, concentrated and the residue purified with 100-200 silica gel, eluting with 15% ethyl acetate in petroleum ether, to afford tert-butyl 3-[1-[1-(2,6-dibenzyloxy-3-pyridyl)-3-methyl-2-oxo-benzimidazol-5-yl]-4-piperidyl]propanoate (3, 1.5 g, 2.22 mmol, 64% yield) as pale yellow solid.

LCMS (ES+): m/z 650.00 [M+H]⁺

### Step 2: tert-butyl 3-[1-[1-(2,6-dioxo-3-piperidyl)-3-methyl-2-oxo-benzimidazol-5-yl]-4-piperidyl]propanoate (4)

A solution of tert-butyl 3-[1-[1-(2,6-dibenzyloxy-3-pyridyl)-3-methyl-2-oxo-benzimidazol-5-yl]-4-piperidyl]propanoate (3, 3 g, 4.62 mmol) in Ethanol (15 mL) and EtOAc (15 mL) was purged with nitrogen gas for 5 min. Then 10% Pd/C (6 g) was added and the resulting mixture was stirred in a Parr shaker (H₂ gas, 80 psi) for 48 h. The reaction mixture was diluted with ethyl acetate (100 mL) and the resulting mixture was filtered through Celite. The solvent was removed to afford tert-butyl 3-[1-[1-(2,6-dioxo-3-piperidyl)-3-methyl-2-oxo-benzimidazol-5-yl]-4-piperidyl]propanoate (4, 1.2 g, 2.47 mmol, 53% yield) as off white solid. The material was used in the next step without further purification.

LCMS (ES+): m/z 471.52 [M+H]⁺

### 5-bromo-1-(2,6-dibenzyloxy-3-pyridyl)-3-(oxetan-3-yl)benzimidazol-2-one (7)

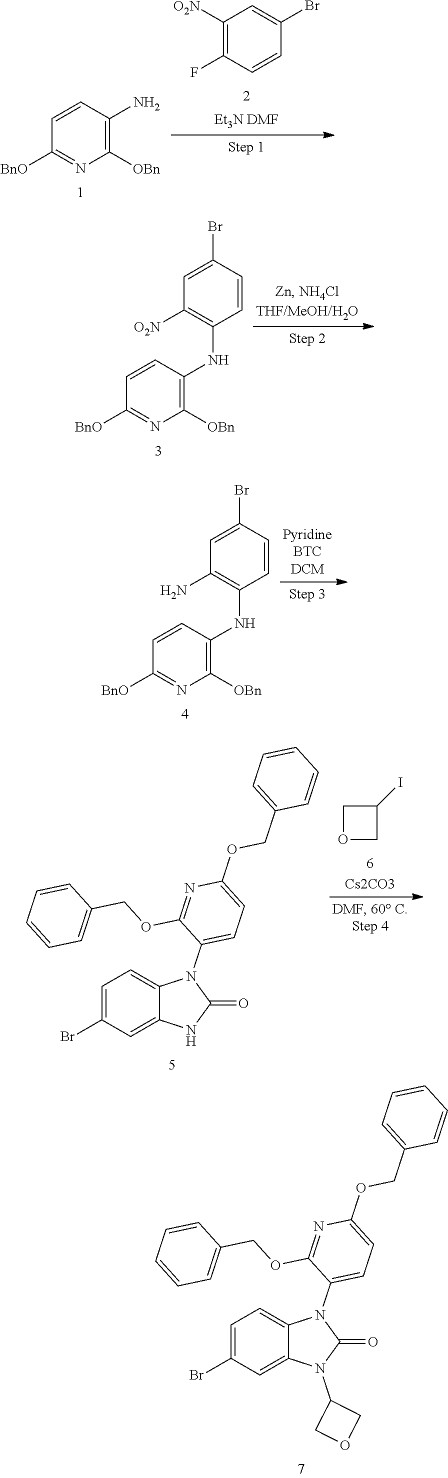

### Step 1: 2,6-dibenzyloxy-N-(4-bromo-2-nitro-phenyl)pyridin-3-amine (3)

A solution of 4-bromo-1-fluoro-2-nitro-benzene (2, 24.70 g, 112.29 mmol, 13.80 mL) and 2,6-dibenzyloxypyridin-3-amine (1, 36.60 g, 112.29 mmol) in anhydrous DMF (419.74 mL) was added to a 250 mL sealed tube. Triethylamine (56.81 g, 561.46 mmol, 78.26 mL) was added and the reaction was heated at 110° C. for 16 h. The reaction mixture was diluted with water (150 mL) and extracted with ethyl acetate (300 mL). The organic layer was washed with water (3×150 mL), dried over sodium sulfate, and concentrated under reduced pressure and purified by column chromatography (silica gel, ethyl acetate/pet ether) to afford 2,6-dibenzyloxy-N-(4-bromo-2-nitro-phenyl)pyridin-3-amine (3, 26 g, 37.48 mmol, 33% yield) as a red-colored solid.

LCMS (ES⁺): m/z 508.4 [M+H]+

### Step 2: 4-bromo-N₁-(2,6-dibenzyloxy-3-pyridyl)benzene-1,2-diamine (4)

A solution of 2,6-dibenzyloxy-N-(4-bromo-2-nitro-phenyl)pyridin-3-amine (3, 20 g, 28.83 mmol) in THF (151.18 mL), methanol (100.78 mL) and water (50.39 mL) was treated with Zinc powder (325 mesh) (9.43 g, 144.17 mmol) and ammonium chloride (7.71 g, 144.17 mmol). After 2 h at 80° C., the reaction was filtered through Celite, washing with ethyl acetate (500 mL), and concentrated in vacuo. The residue was extracted with ethyl acetate (2×500 mL), and the organic layer was dried over sodium sulfate and concentrated under reduced pressure to afford 4-bromo-N₁-(2,6-dibenzyloxy-3-pyridyl)benzene-1,2-diamine (4, 18.00 g, 23.43 mmol, 81% yield) as a brown colored thick mass. The material was used in the next step without further purification LCMS (ES⁺): m/z 478.1 [M+H]+.

### Step 3: 6-bromo-3-(2,6-dibenzyloxy-3-pyridyl)-1H-benzimidazol-2-one (5)

To a solution of 4-bromo-N1-(2,6-dibenzyloxy-3-pyridyl)benzene-1,2-diamine (4, 18 g, 23.43 mmol) in DCM (525.98 mL) was added triphosgene (13.90 g, 46.85 mmol). After 5 min, the reaction was cooled to 0° C. and pyridine (18.53 g, 234.27 mmol, 18.95 mL) in DCM (50 mL) was added. After 3 h at room temperature, the reaction was quenched with ice while stirring and the product was extracted with DCM (250 mL×3). The organic layer was concentrated to afford 6-bromo-3-(2,6-dibenzyloxy-3-pyridyl)-1H-benzimidazol-2-one (5, 7.00 g, 9.61 mmol, 41% yield) as an off-white solid.

LCMS (ES⁺): m/z 504.3 [M+H]⁺.

### Step 4: 5-bromo-1-(2,6-dibenzyloxy-3-pyridyl)-3-(oxetan-3-yl)benzimidazol-2-one (7)

To a solution of Cesium carbonate (1.62 g, 4.98 mmol) in DMF (10 mL) was added 6-bromo-3-(2,6-dibenzyloxy-3-pyridyl)-1H-benzimidazol-2-one (5, 1 g, 1.99 mmol) and 3-iodooxetane (6, 732.44 mg, 3.98 mmol, 342.26 uL). The mixture was stirred at 60° C. for 16 h. The reaction mixture was diluted with water (20 mL) and extracted with ethyl acetate (20 mL×3). The combined organic layers were washed with brine (50 mL*2), dried over Na₂SO₄, filtered and concentrated under reduced pressure. The reaction mixture was purified by flash silica gel chromatography (60 mL/min, Eluent of 0-100% ethylacetate/petroleum ether gradient, Column: ISCO®; 12 g SepaFlash® Silica Flash Column) to afford 5-bromo-1-(2,6-dibenzyloxy-3-pyridyl)-3-(oxetan-3-yl)benzimidazol-2-one (7, 900 mg, 1.50 mmol, 75% yield) was obtained as a yellow oil.

LCMS (ES+): m/z 558.3 [M+H]⁺

### 3-[6-(3,3-difluoro-4-piperidyl)-1-isopropyl-indazol-3-yl]piperidine-2,6-dione (10)

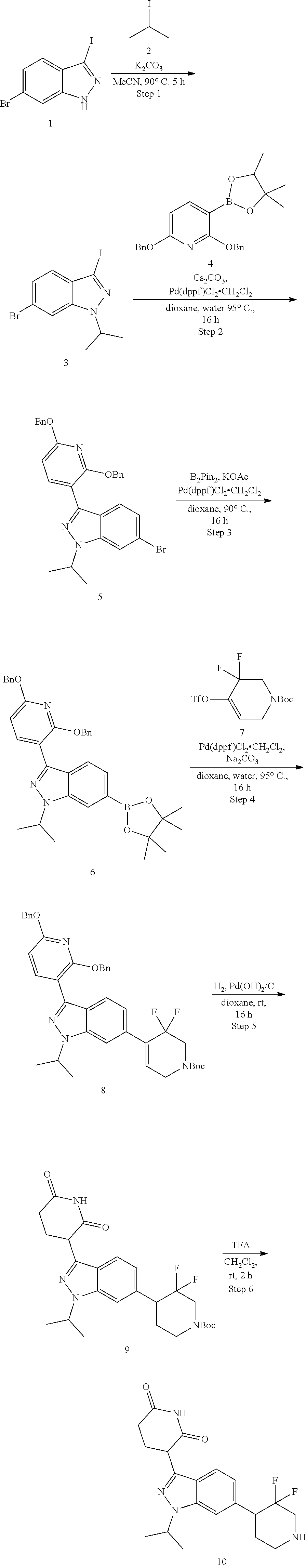

### Step 1: 6-bromo-3-iodo-1-isopropyl-indazole (3)

To a well-stirred solution of 6-bromo-3-iodo-1H-indazole (1, 5 g, 15.48 mmol) in anhydrous acetonitrile (100 mL) were added isopropyl iodide (2, 5.26 g, 30.97 mmol, 3.10 mL) and anhydrous potassium carbonate (6.42 g, 46.45 mmol) at ambient temperature under nitrogen atmosphere and the resulting mixture was heated at 90° C. for 5 h. The reaction mixture was poured into water (50 mL) and extracted with EtOAc (2×50 mL). Organic phases were combined and washed with brine (50 mL). Organic layer was dried over anhydrous Na₂SO₄, filtered and concentrated under reduced pressure. The residue was purified by flash silica-gel (230-400 mesh) chromatography (1% EtOAc in pet ether) to afford a major required regio isomer 6-bromo-3-iodo-1-isopropyl-indazole (3, 3.2 g, 8.30 mmol, 54% yield) as white solid and minor undesired regioisomer 6-bromo-3-iodo-2-isopropyl-indazole (not shown, 1.2 g, 3.08 mmol, 20% yield) as white solid.

LCMS (ES+): m/z 365.0 [M+H]⁺

### Step 2: 6-bromo-3-(2,6-dibenzyloxy-3-pyridyl)-1-isopropyl-indazole (5)

Into a 50 mL sealed tube containing a well-stirred solution of 2,6-dibenzyloxy-3-(4,4,5,5-tetramethyl-1,3,2-dioxaborolan-2-yl)pyridine (4, 851.30 mg, 2.04 mmol) in water (2 mL) were added 6-bromo-3-iodo-1-isopropyl-indazole (3, 745 mg, 2.04 mmol), and cesium carbonate (1.66 g, 5.10 mmol) in 1,4-dioxane (10 mL). The mixture was degassed with nitrogen 10 min. [1,1′-bis(diphenylphosphino)ferrocene]dichloropalladium(II), complex with dichloromethane (166.46 mg, 204.00 μmol) was added and the resulting reaction mixture was heated to 95° C. for 16 h. The reaction was quenched with water (50 mL) and extracted with EtOAc (2×50 mL). The organic phases were combined and washed with brine, dried (anhydrous Na₂SO₄), filtered and concentrated under reduced pressure. The material was purified by flash silica-gel (230-400 mesh) column with (5% EtOAc in pet ether) to afford 6-bromo-3-(2,6-dibenzyloxy-3-pyridyl)-1-isopropyl-indazole (5, 650 mg, 1.06 mmol, 52% yield) as a dark syrup.

LCMS (ES+): m/z 528.0 [M+H]⁺

### Step 3: 3-(2,6-dibenzyloxy-3-pyridyl)-1-isopropyl-6-(4,4,5,5-tetramethyl-1,3,2-dioxaborolan-2-yl) indazole (6)

Into a 50 mL sealed tube containing a well-stirred solution of 6-bromo-3-(2,6-dibenzyloxy-3-pyridyl)-1-isopropyl-indazole (5, 650 mg, 934.83 μmol) in anhydrous 1,4-dioxane (10 mL) were added 4,4,5,5-tetramethyl-2-(4,4,5,5-tetramethyl-1,3,2-dioxaborolan-2-yl)-1,3,2-dioxaborolane (356.09 mg, 1.40 mmol), potassium acetate (183.49 mg, 1.87 mmol, 116.87 μL) and the resulting mixture was degassed with nitrogen for 10 min. 1,1′-bis(diphenylphosphino)ferrocene]dichloropalladium(II), complex with dichloromethane (76.34 mg, 93.48 μmol) was added and the reaction mixture was heated to 90° C. for 16 h. The reaction was quenched with water (100 mL) and extracted with EtOAc (2×100 mL). The organic phases were combined, washed with brine, dried (anhydrous Na₂SO₄), filtered and concentrated under reduced pressure. The material was purified by flash silica-gel (230-400 mesh) column (0-10% EtOAc in pet ether) to afford 3-(2,6-dibenzyloxy-3-pyridyl)-1-isopropyl-6-(4,4,5,5-tetramethyl-1,3,2-dioxaborolan-2-yl) indazole (6, 580 mg, 917.11 μmol, 98% yield) as a syrupy liquid.

LCMS (ES+): m/z 576.2 [M+H]⁺

### Step 4: tert-butyl 4-[3-(2,6-dibenzyloxy-3-pyridyl)-1-isopropyl-indazol-6-yl]-3,3-difluoro-2,6-dihydropyridine-1-carboxylate (8)

Into a 10 mL sealed tube containing a well-stirred solution of 3-(2,6-dibenzyloxy-3-pyridyl)-1-isopropyl-6-(4,4,5,5-tetramethyl-1,3,2-dioxaborolan-2-yl) indazole (6, 100.00 mg, 158.12 μmol) and tert-butyl 3,3-difluoro-4-(trifluoromethylsulfonyloxy)-2,6-dihydropyridine-1-carboxylate (7, 63.13 mg, 158.12 μmol) in dioxane (2 mL) and water (0.2 mL) were added, sodium carbonate (41.90 mg, 395.31 μmol, 16.56 μL) at ambient temperature under nitrogen atmosphere. The resulting mixture was degassed by bubbling nitrogen gas into the reaction mixture for 10 min. Subsequently, 1,1′-bis(diphenylphosphino)ferrocene]dichloropalladium(II), complex with dichloromethane (11.10 mg, 15.81 μmol) was added and the resulting reaction mixture was heated to 95° C. for 16 h. The reaction was quenched with water (20 mL) and extracted with EtOAc (2×30 mL). The organic phases were combined and washed with brine, dried (anhydrous Na₂SO₄), filtered, concentrated under reduced pressure and purified by flash silica-gel (230-400 mesh) column (0-10% EtOAc in pet ether) to afford tert-butyl 4-[3-(2,6-dibenzyloxy-3-pyridyl)-1-isopropyl-indazol-6-yl]-3,3-difluoro-2,6-dihydropyridine-1-carboxylate (8, 80 mg, 99.59 μmol, 63% yield) as a thick syrup.

LCMS (ES+): m/z 667.2 [M+H]⁺

### Step 5: tert-butyl 4-[3-(2,6-dioxo-3-piperidyl)-1-isopropyl-indazol-6-yl]-3,3-difluoro-piperidine-1-carboxylate (9)

Into a 100 mL single neck round bottom flask containing a well-stirred solution of tert-butyl 4-[3-(2,6-dibenzyloxy-3-pyridyl)-1-isopropyl-indazol-6-yl]-3,3-difluoro-2,6-dihydropyridine-1-carboxylate (8, 1.2 g, 1.80 mmol) in 1,4-dioxane (20 mL) was added palladium hydroxide on carbon (20 wt. % 50% water) (500 mg, 3.56 mmol). The resulting suspension was stirred at ambient temperature under hydrogen atmosphere (bladder) for 16 h. The reaction mixture was filtered through Celite and washed with EtOAc (100 mL). The filtrate was concentrated under reduced pressure and triturated with pet ether to afford tert-butyl 4-[3-(2,6-dioxo-3-piperidyl)-1-isopropyl-indazol-6-yl]-3,3-difluoro-piperidine-1-carboxylate (9, 850 mg, 1.65 mmol, 91% yield) as a syrup.

LCMS (ES+): m/z 491.2 [M+H]⁺

### Step 6: 3-[6-(3,3-difluoro-4-piperidyl)-1-isopropyl-indazol-3-yl]piperidine-2,6-dione (10)

Into a 50 mL single neck round bottom flask containing a well-stirred solution of tert-butyl 4-[3-(2,6-dioxo-3-piperidyl)-1-isopropyl-indazol-6-yl]-3,3-difluoro-piperidine-1-carboxylate (9, 850 mg, 162.88 mmol) in anhydrous DCM (10 mL) was added TFA (14.80 g, 129.80 mmol, 10 mL) at 0° C. under nitrogen atmosphere. The resulting mixture was stirred at ambient temperature for 2 h. The reaction mixture was concentrated under reduced pressure and triturated with MTBE to afford 3-[6-(3,3-difluoro-4-piperidyl)-1-isopropyl-indazol-3-yl]piperidine-2,6-dione (10, 0.8 g, 1.49 mmol, 91% yield, TFA salt) as an off-white solid.

LCMS (ES+): m/z 391.2 [M+H]⁺

### 2-[(3S)-4-[1-(2,6-dioxo-3-piperidyl)-3-methyl-2-oxo-benzimidazol-5-yl]-3-methyl-piperazin-1-yl]acetic acid (8)

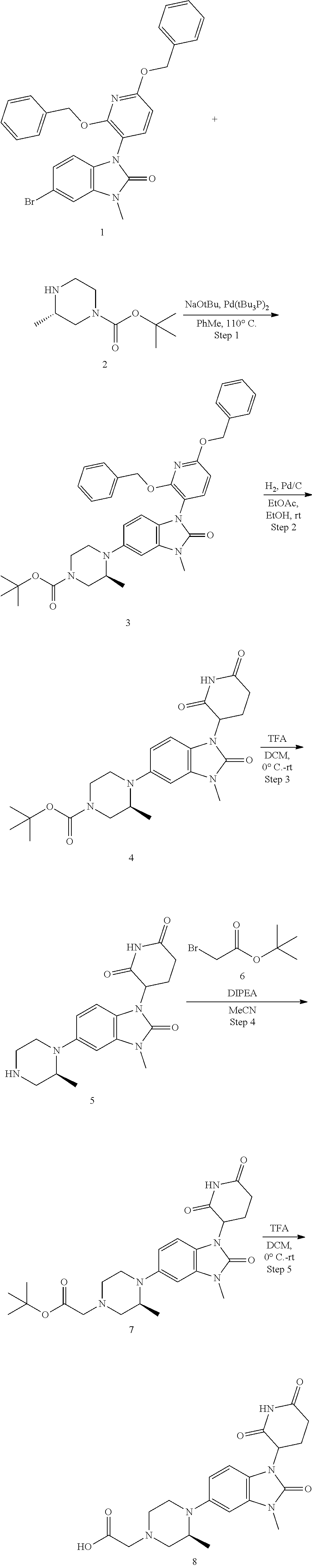

### Step 1: tert-butyl (3S)-4-[1-(2,6-dibenzyloxy-3-pyridyl)-3-methyl-2-oxo-benzimidazol-5-yl]-3-methyl-piperazine-1-carboxylate (3)

To a stirred solution of the 5-bromo-1-(2,6-dibenzyloxy-3-pyridyl)-3-methyl-benzimidazol-2-one (1, 7 g, 13.56 mmol) in toluene (100 mL) was added tert-butyl (3S)-3-methylpiperazine-1-carboxylate (2, 2.71 g, 13.56 mmol) and sodium tert-butoxide (2.61 g, 27.11 mmol). The reaction mixture was purged with nitrogen gas for 10 min. Pd(t-Bu₃P)₂ (692.77 mg, 1.36 mmol) was then added and the mixture purged with nitrogen gas for an additional 5 min before heating at 110° C. for 2 h. Upon completion of the reaction, the mixture was extracted with EtOAc and water, washed with brine, dried over anhydrous sodium sulfate, and filtered. The solvent was removed, and the residue was purified by column chromatography using 100-200 mesh silica gel and 10-50% ethyl acetate/pet ether as eluent to afford tert-butyl (3S)-4-[1-(2,6-dibenzyloxy-3-pyridyl)-3-methyl-2-oxo-benzimidazol-5-yl]-3-methyl-piperazine-1-carboxylate (3, 4.3 g, 6.53 mmol, 48% yield).

LCMS (ES⁺): m/z 636.47 [M+H]⁺

### Step 2: tert-butyl (3S)-4-[1-(2,6-dioxo-3-piperidyl)-3-methyl-2-oxo-benzimidazol-5-yl]-3-methyl-piperazine-1-carboxylate (4)

To a stirred solution of tert-butyl (3S)-4-[1-(2,6-dibenzyloxy-3-pyridyl)-3-methyl-2-oxo-benzimidazol-5-yl]-3-methyl-piperazine-1-carboxylate (3, 0.1 g, 157.29 μmol) in EtOAc (15 mL) and ethanol (15 mL) was added 10% Pd/C (0.09 g, 846.24 μmol). The reaction mixture was stirred at 60° C. for 16 h under hydrogen atmosphere. After completion of the reaction, the mixture was filtered through Celite and concentrated under reduced pressure to afford tert-butyl (3S)-4-[1-(2,6-dioxo-3-piperidyl)-3-methyl-2-oxo-benzimidazol-5-yl]-3-methyl-piperazine-1-carboxylate (4, 0.08 g, 30.42 μmol, 19% yield).

LCMS (ES⁺): m/z 458.36 [M+H]⁺.

### Step 3: 3-[3-methyl-5-[(2S)-2-methylpiperazin-1-yl]-2-oxo-benzimidazol-1-yl]piperidine-2,6-dione (5)

To a solution of tert-butyl (3S)-4-[1-(2,6-dioxo-3-piperidyl)-3-methyl-2-oxo-benzimidazol-5-yl]-3-methyl-piperazine-1-carboxylate (4, 1.3 g, 2.84 mmol) in DCM (15 mL), trifluoroacetic acid (1.62 g, 14.21 mmol, 1.09 mL) was added. The reaction mixture was stirred for 2 h at 25° C. After completion of the reaction, the reaction mixture was concentrated under reduced pressure and washed with diethyl ether twice to afford 3-[3-methyl-5-[(2S)-2-methylpiperazin-1-yl]-2-oxo-benzimidazol-1-yl]piperidine-2,6-dione (5, 0.98 g, 2.01 mmol, 71% yield, TFA salt).

LCMS (ES⁺): m/z 358.37 [M+H]⁺.

### Step 4: tert-butyl 2-[(3S)-4-[1-(2,6-dioxo-3-piperidyl)-3-methyl-2-oxo-benzimidazol-5-yl]-3-methyl-piperazin-1-yl]acetate (7)

To a mixture of 3-[3-methyl-5-[(2S)-2-methylpiperazin-1-yl]-2-oxo-benzimidazol-1-yl]piperidine-2,6-dione (5, 1.5 g, 3.18 mmol, TFA salt) in ACN (20 mL) and N,N-diisopropylethylamine (2.88 g, 22.27 mmol, 3.88 mL), tert-butyl 2-bromoacetate (6, 620.62 mg, 3.18 mmol, 466.63 μL) was added. The reaction was stirred for 16 h at 70° C. After completion of the reaction, the reaction mixture was diluted with water and extracted with ethyl acetate twice. The combined organic layer was dried over sodium sulfate, filtered, and concentrated under reduced pressure to give the crude product, which was purified by column chromatography (Devisil silica) to afford tert-butyl 2-[(3S)-4-[1-(2,6-dioxo-3-piperidyl)-3-methyl-2-oxo-benzimidazol-5-yl]-3-methyl-piperazin-1-yl]acetate (7, 0.600 g, 1.23 mmol, 39% yield).

### Chiral HPLC Method Conditions:

Analytical SFC Conditions: Column/dimensions: Chiralcel OJ-3 (4.6×150) mm, 3 g % C02:75% % Co solvent: 25% (Aetonitrile)

Total Flow: 3.00 g/min Back Pressure: 1500 PSI

Temperature (degree): 30

LCMS (ES⁺): m/z 472.39 [M+H]⁺.

### Step 5: 2-[(3S)-4-[1-(2,6-dioxo-3-piperidyl)-3-methyl-2-oxo-benzimidazol-5-yl]-3-methyl-piperazin-1-yl]acetic acid (8)

In a 50 mL single neck round bottom flask, a well-stirred solution of tert-butyl 2-[(3S)-4-[1-(2,6-dioxo-3-piperidyl)-3-methyl-2-oxo-benzimidazol-5-yl]-3-methyl-piperazin-1-yl]acetate (7, 160 mg, 325.74 μmol) in anhydrous DCM (2.50 mL) was added TFA (1.52 g, 13.29 mmol, 1.02 mL) at 0° C. under nitrogen atmosphere. The resulting mixture was stirred at ambient temperature for 16 h. The reaction mixture was concentrated under reduced pressure to get a crude product. The crude product was triturated with pet ether to afford 2-[(3S)-4-[1-(2,6-dioxo-3-piperidyl)-3-methyl-2-oxo-benzimidazol-5-yl]-3-methyl-piperazin-1-yl]acetic acid (8, 160 mg, 296.15 μmol, 91% yield, TFA salt) as an off white solid.

LCMS (ES⁺): m/z 416.2 [M+H]⁺.

### 2-[(3R)-4-[1-(2,6-dioxo-3-piperidyl)-3-methyl-2-oxo-benzimidazol-5-yl]-3-methyl-piperazin-1-yl]acetic acid (1)

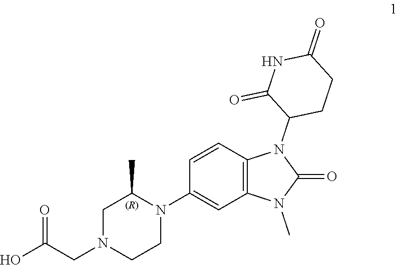

2-[(3R)-4-[1-(2,6-dioxo-3-piperidyl)-3-methyl-2-oxo-benzimidazol-5-yl]-3-methyl-piperazin-1-yl]acetic acid (100 mg, 174.86 μmol, 82% yield, TFA salt) was prepared following the same 5 step procedure as 2-[(3S)-4-[1-(2,6-dioxo-3-piperidyl)-3-methyl-2-oxo-benzimidazol-5-yl]-3-methyl-piperazin-1-yl]acetic acid.

LCMS (ES+): m/z 416.2 [M+H]⁺

### 3-[4-chloro-3-methyl-2-oxo-5-(4-piperidyl)benzimidazol-1-yl]piperidine-2,6-dione

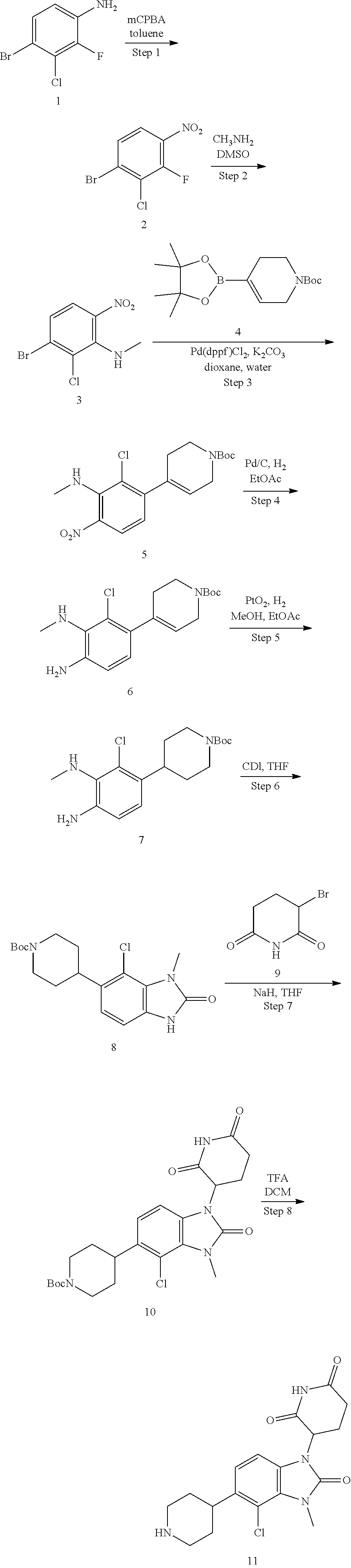

### Step 1:1-bromo-2-chloro-3-fluoro-4-nitrobenzene (2)

To stirred solution of 4-bromo-3-chloro-2-fluoro-aniline (1, 30 g, 133.66 mmol) in toluene (375 mL), 3-chlorobenzenecarboperoxoic acid (92.26 g, 534.62 mmol) was added slowly at room temperature. After complete addition of 3-chlorobenzenecarboperoxoic acid, the reaction was stirred at 50-55° C. for 16 h. Dichloromethane was added to the reaction mixture and then filtered. The filtrate was concentrated and then purified by flash column chromatography to get the desired product 1-bromo-2-chloro-3-fluoro-4-nitrobenzene (2, 7.6 g, 26.77 mmol, 20% yield). The material was used in the next step without further characterization.

### Step 2: 3-bromo-2-chloro-N-methyl-6-nitroaniline (3)

To a stirred solution of 1-bromo-2-chloro-3-fluoro-4-nitrobenzene (2, 7.6 g, 29.87 mmol) in DMSO (15 mL), methylamine, 40% w/w aq. soln. (4.64 g, 149.35 mmol, 5.16 mL) was added slowly at room temperature. The reaction mixture was stirred for 2 h. The reaction mixture was diluted with ice cold water, where a solid precipitates was formed. The solid was filtered off, washed with water, and dried under vacuum to give the desired product 3-bromo-2-chloro-N-methyl-6-nitroaniline (3, 7.5 g, 26.29 mmol, 88% yield). LCMS (ES⁺): m/z 265.09 [M+H]⁺.

### Step 3: tert-butyl 4-(2-chloro-3-(methylamino)-4-nitrophenyl)-3,6-dihydropyridine-1 (2H)-carboxylate (5)

To a stirred solution of 3-bromo-2-chloro-N-methyl-6-nitroaniline (3, 7.5 g, 28.25 mmol) in 1,4-dioxane (50 mL) and water (50 mL), potassium carbonate, anhydrous, 99% (11.71 g, 84.75 mmol) and tert-butyl 4-(4,4,5,5-tetramethyl-1,3,2-dioxaborolan-2-yl)-3,6-dihydropyridine-1 (2H)-carboxylate (4, 8.73 g, 28.25 mmol) were added. The reaction mixture was purged with argon gas for 10-15 min before [1,1′-Bis(diphenylphosphino)ferrocene]dichloropalladium(II) dichloromethane (1:1) (1.15 g, 1.41 mmol) was added. The reaction mixture was allowed to stir for 2 h at 100-110° C. under argon atmosphere. The reaction was quenched with brine solution and extracted with ethyl acetate. It was dried over sodium sulfate and concentrated to get the crude product, which was purified by flash column chromatography to afford tert-butyl 4-(2-chloro-3-(methylamino)-4-nitrophenyl)-3,6-dihydropyridine-1 (2H)-carboxylate (5, 9.1 g, 24.10 mmol, 85% yield). LCMS (ES⁺): m/z 368.28 [M+H]⁺.

### Step 4: tert-butyl 4-(4-amino-2-chloro-3-(methylamino)phenyl)-3,6-dihydropyridine-1 (2H)-carboxylate (6)

In a round-bottom flask, a solution of tert-butyl 4-(2-chloro-3-(methylamino)-4-nitrophenyl)-3,6-dihydropyridine-1 (2H)-carboxylate (5, 500 mg, 1.36 mmol) in ethyl acetate was added palladium, 10% on carbon, Type 487, dry (144.66 mg, 1.36 mmol) at room temperature. The reaction was stirred for 5 h at 10 psi pressure. The reaction mixture was filtered through Celite and concentrated to get the crude product, which was purified by flash column chromatography to afford tert-butyl 4-(4-amino-2-chloro-3-(methylamino)phenyl)-3,6-dihydropyridine-1 (2H)-carboxylate (6, 350 mg, 642.31 μmol, 47% yield). LCMS (ES⁺): m/z 338.30 [M+H]⁺.

### Step 5: tert-butyl 4-(4-amino-2-chloro-3-(methylamino)phenyl)piperidine-1-carboxylate (7)

To a solution of tert-butyl 4-(4-amino-2-chloro-3-(methylamino)phenyl)-3,6-dihydropyridine-1 (2H)-carboxylate (6, 2.68 g, 7.93 mmol) in methanol (50 mL) and ethyl acetate (50 mL) was added platinum (IV) oxide (1.80 g, 7.93 mmol). The reaction mixture was stirred at room temperature for 7 h. The mixture was filtered through Celite, and the filtrate was concentrated in vacuo. It was then purified by flash column chromatography to afford tert-butyl 4-(4-amino-2-chloro-3-(methylamino)phenyl)piperidine-1-carboxylate (7, 1.9 g, 4.91 mmol, 62% yield).

LCMS (ES⁺): m/z 340.31 [M+H]⁺.

### Step 6: tert-butyl 4-(4-chloro-3-methyl-2-oxo-2,3-dihydro-1H-benzo[d]imidazol-5-yl)piperidine-1-carboxylate (8)

To a stirred solution of tert-butyl 4-(4-amino-2-chloro-3-(methylamino)phenyl)piperidine-1-carboxylate (7, 5.5 g, 16.18 mmol) in THF (55 mL), di(imidazol-1-yl)methanone (7.87 g, 48.55 mmol) was added at room temperature. The reaction mixture was stirred for 12 h at 65° C. The reaction mixture was evaporated and the residue was diluted with water and stirred for 15 min. It was then filtered, washed with n-hexane and dried to get the desired compound tert-butyl 4-(4-chloro-3-methyl-2-oxo-2,3-dihydro-1H-benzo[d]imidazol-5-yl)piperidine-1-carboxylate (8, 4.5 g, 11.70 mmol, 72% yield). LCMS (ES⁻): m/z 364.23[M−H]⁻.

### Step 7: tert-butyl 4-(4-chloro-1-(2,6-dioxopiperidin-3-yl)-3-methyl-2-oxo-2,3-dihydro-1H-benzo[d]imidazol-5-yl)piperidine-1-carboxylate (10)

To a solution of sodium hydride (60% dispersion in mineral oil) (502.71 mg, 21.87 mmol) in THF (150 mL), tert-butyl 4-(4-chloro-3-methyl-2-oxo-2,3-dihydro-1H-benzo[d]imidazol-5-yl)piperidine-1-carboxylate (8, 4 g, 10.93 mmol) was added portionwise at room temperature. Then the reaction mixture was stirred at 60° C. After 30 min, 3-bromopiperidine-2,6-dione (9, 5.25 g, 27.33 mmol) in THF was added slowly and reaction was stirred for another 5 h at 60° C. The reaction was cooled to room temperature and methanol was added to quench the reaction. Then it was washed with brine and the organic layer was extracted with ethyl acetate. It was dried over anhydrous sodium sulfate and concentrated to get the desired crude, which was then purified by flash column chromatography to get the desired product tert-butyl 4-(4-chloro-1-(2,6-dioxopiperidin-3-yl)-3-methyl-2-oxo-2,3-dihydro-1H-benzo[d]imidazol-5-yl)piperidine-1-carboxylate (10, 1.9 g, 3.76 mmol, 34% yield). LCMS (ES⁻): m/z 475.24 [M−H]⁻.

### Step 8: 3-(4-chloro-3-methyl-2-oxo-5-(piperidin-4-yl)-2,3-dihydro-1H-benzo[d]imidazol-1-yl)piperidine-2,6-dione (11)

To a stirred solution of tert-butyl 4-(4-chloro-1-(2,6-dioxopiperidin-3-yl)-3-methyl-2-oxo-2,3-dihydro-1H-benzo[d]imidazol-5-yl)piperidine-1-carboxylate (10, 50 mg, 104.83 μmol) in DCM (3 mL), trifluoroacetic acid (1.48 g, 12.98 mmol, 1.00 mL) was added at 0° C. Then the reaction was stirred at room temperature for 2 h. The reaction was concentrated and triturated with diethyl ether and hexane. It was concentrated under high vacuum to afford 3-(4-chloro-3-methyl-2-oxo-5-(piperidin-4-yl)-2,3-dihydro-1H-benzo[d]imidazol-1-yl)piperidine-2,6-dione (11, 50 mg, 94.94 μmol, 91% yield, TFA salt). LCMS (ES⁺): m/z 377.21 [M+H]⁺.

### 3-[5-(8-azabicyclo[3.2.1]octan-3-yl)-3-methyl-2-oxo-benzimidazol-1-yl]piperidine-2,6-dione (13)

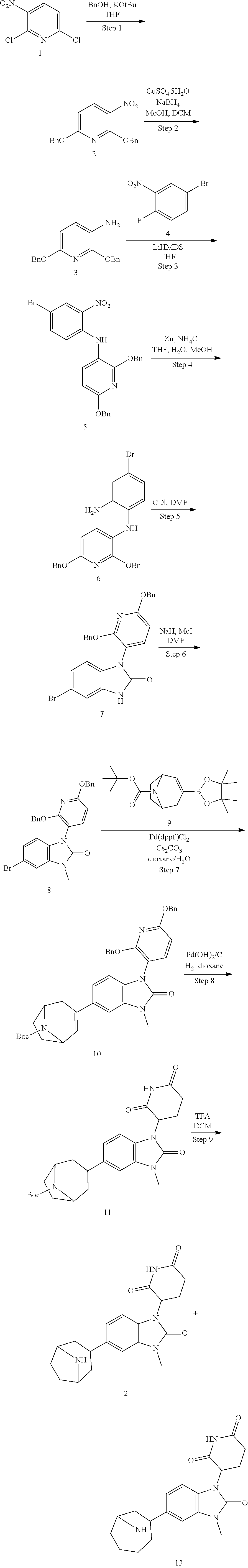

### Step 1: 2,6-dibenzyloxy-3-nitro-pyridine (2)

In a 1 L three neck round bottom flask, a stirred solution of potassium tert-butoxide (39.25 g, 349.76 mmol) in anhydrous THF (100 mL) was cooled to −15° C. and benzyl alcohol (33.62 g, 310.90 mmol, 32.17 mL) was added over 10 min under nitrogen atmosphere. The resulting reaction mixture was warmed to 20-22° C. and stirred for another h. The reaction mixture was cooled to −20° C., then slowly added a solution of 2,6-dichloro-3-nitro-pyridine (1, 25 g, 129.54 mmol) in anhydrous THF (150 mL). The reaction was quenched with ice cold water. Then the resulting precipitate was filtered out on a Buchner funnel. The filter cake was thoroughly washed with water (100 mL×2) and dried to afford 2,6-dibenzyloxy-3-nitro-pyridine (2, 30 g, 89.20 mmol, 69% yield) as a yellow colored solid.

LCMS: m/z (ES⁺): 337.4 [M+H]+.

### Step 2: 2,6-dibenzyloxypyridin-3-amine (3)

In a 1000 ml three neck round bottom flask, a well-stirred solution of copper sulfate pentahydrate (8.91 g, 35.68 mmol) in dry methanol (200 mL) was added a solution of 2,6-dibenzyloxy-3-nitro-pyridine (2, 30 g, 89.20 mmol) in anhydrous DCM (200 mL). The reaction mixture was cooled to −15° C. then sodium borohydride (20.25 g, 535.17 mmol) was added portionwise. The reaction mixture was stirred for an additional two h at −5° C. The reaction mixture was quenched with ice water (50 mL) and the volatiles were evaporated off. The crude product was extracted with ethyl acetate and water (2×200 mL). The organic layer was evaporated off and azeotroped with toluene (20 mL). The resulting black colored crude was purified by column chromatography (230-400 mesh silica gel) using 10% EtOAc-Pet ether as eluent to afford 2,6-dibenzyloxypyridin-3-amine (3, 19.5 g, 62.38 mmol, 70% yield) as a brown colored dense liquid.

LCMS (ES⁺): m/z 307.4 [M+H]⁺.

### Step 3: 2,6-dibenzyloxy-N-(4-bromo-2-nitro-phenyl)pyridin-3-amine (5)

A solution of 2,6-dibenzyloxypyridin-3-amine (3, 100 g, 326.42 mmol) in THF (500 mL) was cooled to −78° C. and lithium bis(trimethylsilyl)amide (81.93 g, 489.62 mmol, 500 mL) as added. The reaction mixture was stirred for 1 h and 4-bromo-1-fluoro-2-nitro-benzene (4, 71.81 g, 326.42 mmol, 40.12 mL) was added. The reaction mixture was concentrated in vacuo, diluted with water and extracted with ethyl acetate. The organic layer was washed with brine solution, dried over sodium sulfate and concentrated in vacuo to afford crude 2,6-dibenzyloxy-N-(4-bromo-2-nitro-phenyl)pyridin-3-amine (5, 130 g, 230.63 mmol, 71% yield). LCMS (ES⁺): m/z 506.10 [M+H]⁺.

### Step 4: 4-bromo-N1-(2,6-dibenzyloxy-3-pyridyl)benzene-1,2-diamine (6)

To a stirred solution of 2,6-dibenzyloxy-N-(4-bromo-2-nitro-phenyl)pyridin-3-amine (5, 200 g, 394.99 mmol) in THF (4000 mL) was added zinc (258.28 g, 3.95 mol, 36.17 mL) and ammonium chloride (422.57 g, 7.90 mol, 276.19 mL) portionwise at 0-28° C. Reaction mixture was filtered through Celite, the filtrate was concentrated in vacuo and extracted with ethyl acetate, washed with brine solution and dried over sodium sulfate. It was then concentrated to dryness to afford 4-bromo-N₁-(2,6-dibenzyloxy-3-pyridyl)benzene-1,2-diamine (6, 150 g, 292.87 mmol, 74% yield).

LCMS (ES⁺): m/z 476.22 [M+H]⁺.

### Step 5: 6-bromo-3-(2,6-dibenzyloxy-3-pyridyl)-1H-benzimidazol-2-one (7)

To a stirred solution of 4-bromo-N1-(2,6-dibenzyloxy-3-pyridyl)benzene-1,2-diamine (6, 110 g, 230.92 mmol) in DMF (1000 mL) was added 1,1′-carbonyldiimidazole (112.33 g, 692.75 mmol) and the reaction mixture stirred at 0-28° C. for 16 h. The reaction mixture was diluted with ice cold water, and stirred for 1 h. The solid obtained was filtered and dried under vacuum to afford 6-bromo-3-(2,6-dibenzyloxy-3-pyridyl)-1H-benzimidazol-2-one (7, 100 g, 181.01 mmol, 78% yield).

LCMS (ES⁺): m/z 502.06 [M+H]⁺.

### Step 6: 5-bromo-1-(2,6-dibenzyloxy-3-pyridyl)-3-methyl-benzimidazol-2-one (8)

To a stirred solution of 6-bromo-3-(2,6-dibenzyloxy-3-pyridyl)-1H-benzimidazol-2-one (7, 108 g, 214.99 mmol) in DMF (1000 mL) was added sodium hydride (60% dispersion in mineral oil) (14.83 g, 644.96 mmol) portionwise at 0-28° C. and the reaction mixture was stirred for 1 h. Methyl iodide (D3) stored over copper (31.16 g, 214.99 mmol, 13.37 mL) was added dropwise at half an h. Reaction mixture was diluted with ice cold water, and the solid obtained was filtered and dried under vacuum. It was then extracted with ethyl acetate, washed with brine solution, dried over sodium sulfate, and concentrated in vacuo to give the crude product, which was washed with pentane to afford 5-bromo-1-(2,6-dibenzyloxy-3-pyridyl)-3-methyl-benzimidazol-2-one (8, 95 g, 183.81 mmol, 86% yield).

LCMS (ES⁺): m/z 516.14 [M+H]⁺.

### Step 7: tert-butyl 3-(1-(2,6-bis(benzyloxy)pyridin-3-yl)-3-methyl-2-oxo-2,3-dihydro-1H-benzo[d]imidazol-5-yl)-8-azabicyclo[3.2.1]oct-2-ene-8-carboxylate (10)

To a stirred solution of 1-(2,6-bis(benzyloxy)pyridin-3-yl)-5-bromo-3-methyl-1,3-dihydro-2H-benzo[d]imidazol-2-one (8, 500 mg, 968.27 μmol) in 1,4-dioxane (16 mL) and water (4 mL), tert-butyl 3-(4,4,5,5-tetramethyl-1,3,2-dioxaborolan-2-yl)-8-azabicyclo[3.2.1]oct-2-ene-8-carboxylate (9, 421.99 mg, 1.26 mmol) and cesium carbonate (946.44 mg, 2.90 mmol) were added at room temperature under argon atmosphere. Then the reaction mixture was purged with argon gas for 15 min followed by the addition of [1,1′-bis(diphenylphosphino)ferrocene]dichloropalladium(II) (48.41 μmol). The reaction was stirred at 80° C. for 18 h under argon atmosphere. After completion of the reaction, it was quenched by adding brine and the aqueous layer was extracted with ethyl acetate, dried over sodium sulfate, and filtered. It was then concentrated in vacuo to give the crude product, which was purified by flash column chromatography to get the desired product tert-butyl 3-(1-(2,6-bis(benzyloxy)pyridin-3-yl)-3-methyl-2-oxo-2,3-dihydro-1H-benzo[d]imidazol-5-yl)-8-azabicyclo[3.2.1]oct-2-ene-8-carboxylate (10, 445 mg, 658.09 μmol, 68% yield).

LCMS (ES⁺): m/z 645.33 [M+H]⁺.

### Step 8: tert-butyl 3-(1-(2,6-dioxopiperidin-3-yl)-3-methyl-2-oxo-2,3-dihydro-1H-benzo[d]imidazol-5-yl)-8-azabicyclo[3.2.1]octane-8-carboxylate (11)

In a steel bomb, a solution of tert-butyl 3-(1-(2,6-bis(benzyloxy)pyridin-3-yl)-3-methyl-2-oxo-2,3-dihydro-1H-benzo[d]imidazol-5-yl)-8-azabicyclo[3.2.1]oct-2-ene-8-carboxylate (10, 3.2 g, 4.96 mmol) in 1,4-dioxane (160 mL), palladium hydroxide on carbon, 20 wt. % 50% water (1.60 g, 11.39 mmol) was added at room temperature. The reaction was stirred for 24 h at 60° C. under hydrogen atmosphere (150 psi). After completion of the reaction, it was filtered through Celite and the filtrate was concentrated to get the desired crude product. It was triturated with diethyl ether to afford tert-butyl 3-(1-(2,6-dioxopiperidin-3-yl)-3-methyl-2-oxo-2,3-dihydro-1H-benzo[d]imidazol-5-yl)-8-azabicyclo[3.2.1]octane-8-carboxylate (11, 2.2 g, 4.36 mmol, 88% yield).

LCMS (ES−): m/z 467.22 [M−H]⁻.

### Step 9: 3-(5-(8-azabicyclo[3.2.1]octan-3-yl)-3-methyl-2-oxo-2,3-dihydro-1H-benzo[d]imidazol-1-yl)piperidine-2,6-dione isomers (12, 13)

To a stirred solution of tert-butyl 3-(1-(2,6-dioxopiperidin-3-yl)-3-methyl-2-oxo-2,3-dihydro-1H-benzo[d]imidazol-5-yl)-8-azabicyclo[3.2.1]octane-8-carboxylate (11, 2.22 g, 4.74 mmol) in DCM (12 mL), trifluoroacetic acid (8.96 g, 78.59 mmol, 6.05 mL) was added. The reaction was concentrated in vacuo to get the crude product. Crude product was triturated with diethyl ether to get the mixture of two isomers. It was then separated by reverse-phase HPLC to get the isomer-1 3-(5-(8-azabicyclo[3.2.1]octan-3-yl)-3-methyl-2-oxo-2,3-dihydro-1H-benzo[d]imidazol-1-yl)piperidine-2,6-dione (12, 0.49 g, 975.53 μmol, 21% yield, TFA salt) and isomer-2 3-(5-(8-azabicyclo[3.2.1]octan-3-yl)-3-methyl-2-oxo-2,3-dihydro-1H-benzo[d]imidazol-1-yl)piperidine-2,6-dione (13, 0.47 g, 970.00 μmol, 20% yield, TFA salt).

Isomer 1 (12): LCMS (ES⁺): m/z 369.30 [M+H]⁺.

Isomer 2 (13): LCMS (ES⁺): m/z 369.30 [M+H]⁺.

### tert-butyl 4-[[1-(2,6-dioxo-3-piperidyl)-2-oxo-benzo[cd]indol-6-yl]methylamino]-4-oxo-butanoate (9)

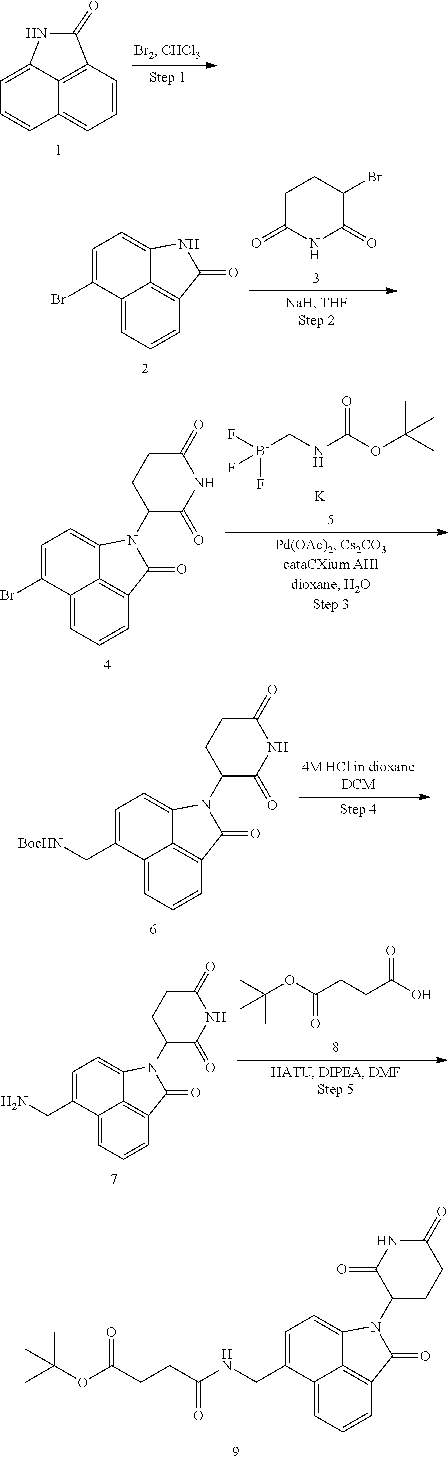

### Step 1: 6-bromo-1H-benzo[cd]indol-2-one (2)

Into a 2 L three-neck round-bottom flask containing a well-stirred solution of 1H-benzo[cd]indol-2-one (1, 20.00 g, 118.22 mmol) in chloroform (1.2 L), cooled to 0° C., was added bromine (14.36 g, 177.52 mmol, 9.64 mL) dropwise at 0° C. The reaction mixture was stirred at room temperature for 20 h. The reaction was quenched carefully with sodium thiosulfate solution (1000 mL) in cold condition and the resulting yellow solid was filtered. Then the solid was washed with cold water (250 mL) and diethyl ether (150 mL) to afford 6-bromo-1H-benzo[cd]indol-2-one (2, 22.0 g, 82.56 mmol, 70% yield) as a yellow solid. LCMS (ES⁺): m/z 250.0 [M+2]⁺.

### Step 2: 3-(6-bromo-2-oxo-benzo[cd]indol-1-yl)piperidine-2,6-dione (4)

Into a 500 mL three-neck round-bottom flask containing a well-stirred solution of 6-bromo-1H-benzo[cd]indol-2-one (2, 2.50 g, 10.08 mmol) in THF (200 mL), cooled to 0° C., sodium hydride (60% dispersion in mineral oil) (2.00 g, 52.20 mmol) was added and the resulting mixture was stirred room temperature for 1 h. To this, 3-bromopiperidine-2,6-dione (3, 4.84 g, 25.19 mmol) in THF (10 mL) was added dropwise at 0° C. and the mixture was heated at 60° C. for 16 h. The reaction was slowly quenched at 0° C. with saturated NH₄Cl solution and extracted with ethyl acetate (2×100 mL). The combined organic layer was dried over anhydrous Na₂SO₄, filtered and concentrated under reduced pressure. The product was recrystallized with dichloromethane (10 mL) and dried to afford 3-(6-bromo-2-oxo-benzo[cd]indol-1-yl)piperidine-2,6-dione (4, 2.5 g, 6.31 mmol, 63% yield) as a yellow solid. LCMS (ES⁺): m/z 360.8 [M+H]⁺.

### Step 3: tert-butyl N-[[1-(2,6-dioxo-3-piperidyl)-2-oxo-benzo[cd]indol-6-yl]methyl]carbamate (6)

Into a 250 mL sealed-tube containing a well-stirred solution of 3-(6-bromo-2-oxo-benzo[cd]indol-1-yl)piperidine-2,6-dione (4, 2.00 g, 5.57 mmol) and potassium [[(tert-butoxycarbonyl)amino]methyl]trifluoroborate (5, 3.30 g, 13.92 mmol) in 1,4-dioxane (60 mL), were added cesium carbonate (5.44 g, 16.71 mmol) and water (8 mL). The reaction mixture was degassed with N₂ for 10 min before palladium (II) acetate (125.01 mg, 556.83 μmol) and di(1-adamantyl)-n-butylphosphine (99.82 mg, 278.42 μmol) were added. The mixture was degassed with nitrogen for 5 min and was heated to 100° C. for 16 h. The mixture was cooled to 0° C., slowly quenched with saturated NH₄Cl solution and extracted with ethyl acetate (2×75 mL). The combined organic layer was dried over anhydrous Na₂SO₄, filtered and concentrated under reduced pressure. The product was purified by flash column chromatography (Silica gel, 230-400 mesh) using 0-100% ethyl acetate in pet ether while the desired compound eluting at 50-60% ethyl acetate in pet ether to afford tert-butyl N-[[1-(2,6-dioxo-3-piperidyl)-2-oxo-benzo[cd]indol-6-yl]methyl]carbamate (6, 900 mg, 1.78 mmol, 32% yield) as a yellow solid. LCMS (ES⁺): m/z 293.1 [M−117+H]⁺.

### Step 4: 3-[6-(aminomethyl)-2-oxo-benzo[cd]indol-1-yl]piperidine-2,6-dione (7)

Into a 100 mL single neck round bottom flask containing a well-stirred solution of tert-butyl N-[[1-(2,6-dioxo-3-piperidyl)-2-oxo-benzo[cd]indol-6-yl]methyl]carbamate (6, 1.4 g, 3.42 mmol) in anhydrous DCM (10 mL), was added 4.0 M hydrogen chloride solution in dioxane (5 mL) at 0° C. The resulting mixture was stirred at rt for 1 h. The solvent was removed and the residue triturated with diethyl ether (10 mL) and dried to afford 3-[6-(aminomethyl)-2-oxo-benzo[cd]indol-1-yl]piperidine-2,6-dione (7, 1.2 g, 2.95 mmol, 86% yield, HCl salt) as an yellow solid.

### Step 5: tert-butyl 4-[[1-(2,6-dioxo-3-piperidyl)-2-oxo-benzo[cd]indol-6-yl]methylamino]-4-oxo-butanoate (9)

Into a 50 mL single neck round bottom flask containing a well-stirred solution of 3-[6-(aminomethyl)-2-oxo-benzo[cd]indol-1-yl]piperidine-2,6-dione (7, 600 mg, 1.94 mmol) and 4-tert-butoxy-4-oxo-butanoic acid (8, 337.89 mg, 1.94 mmol) in DMF (5 mL). The mixture was cooled to 0° C. and DIPEA (752.08 mg, 5.82 mmol, 1.01 mL) and HATU (1.11 g, 2.91 mmol) were added and the mixture was stirred at room temperature for 1 h. The crude mixture was cooled to 0° C., quenched with ice cold water (30 mL) and extracted with ethyl acetate (2×50 mL). The combined organic layer was dried over anhydrous Na₂SO₄, filtered and concentrated under reduced pressure. The material was purified by flash column chromatography (Silica gel, 230-400 mesh) using 0-100% ethyl acetate in pet ether to afford tert-butyl 4-[[1-(2,6-dioxo-3-piperidyl)-2-oxo-benzo[cd]indol-6-yl]methylamino]-4-oxo-butanoate (9, 1.0 g, 1.80 mmol, 93% yield) as a yellow liquid. LCMS (ES⁺): m/z 410.0 [M−56+H]⁺.

### tert-butyl 2-(3-(1-(2,6-bis(benzyloxy)pyridin-3-yl)-3-methyl-2-oxo-2,3-dihydro-1H-benzo[d]imidazol-5-yl)phenyl)acetate (3)

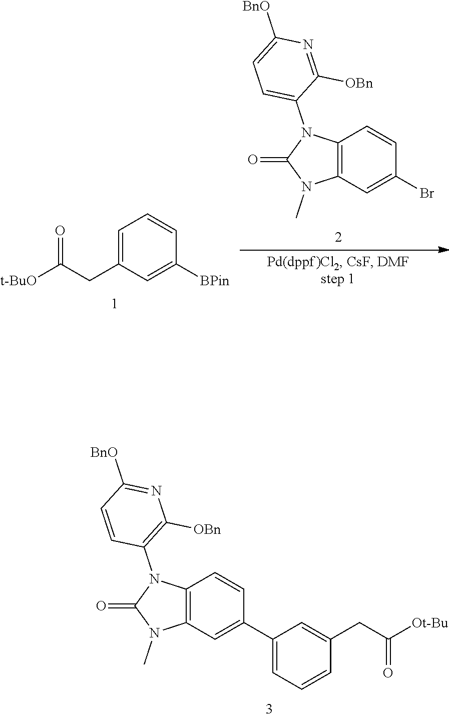

### Step 1: tert-butyl 2-(3-(1-(2,6-bis(benzyloxy)pyridin-3-yl)-3-methyl-2-oxo-2,3-dihydro-1H-benzo[d]imidazol-5-yl)phenyl)acetate (3)

To a solution of 1-(2,6-bis(benzyloxy)pyridin-3-yl)-5-bromo-3-methyl-1H-benzo[d]imidazol-2 (3H)-one (2, 500 mg, 968.27 μmol) and tert-butyl 2-(3-(4,4,5,5-tetramethyl-1,3,2-dioxaborolan-2-yl)phenyl)acetate (1, 450 mg, 1.41 mmol) in DMF (5 mL) were added Pd(dppf)Cl₂ (79.07 mg, 96.83 μmol) and CsF (192.00 mg, 2.90 mmol). The mixture was stirred at 90° C. for 16 h. The reaction mixture was diluted with H₂O (150 mL) and extracted with ethyl acetate (150 mL×3), dried over Na₂SO₄, filtered and concentrated under reduced pressure. The residue was purified by column chromatography (SiO₂, Petroleum ether/Ethyl acetate=1/0 to 1/1) to afford tert-butyl 2-(3-(1-(2,6-bis(benzyloxy)pyridin-3-yl)-3-methyl-2-oxo-2,3-dihydro-1H-benzo[d]imidazol-5-yl)phenyl)acetate (3, 250 mg, 378.35 μmol, 39% yield) as a brown solid.

LCMS (ESI): m/z 628.5 [M+H]⁺

### 3-(5-bromo-3-methyl-indazol-1-yl)piperidine-2,6-dione (4)

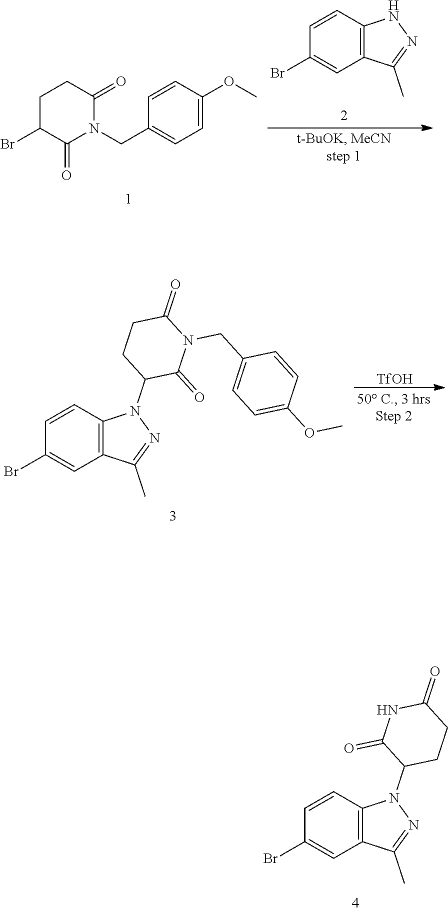

### Step 1: 3-(5-bromo-3-methyl-1H-indazol-1-yl)-1-(4-methoxybenzyl)piperidine-2,6-dione (3)

A mixture of compound 5-bromo-3-methyl-1H-indazole (2, 15.0 g, 71.1 mmol), t-BuOK (16.0 g, 142 mmol) in MeCN (100 mL) was degassed and purged with N₂ for 3 times, and stirred for 1 h, 3-bromo-1-(4-methoxybenzyl)piperidine-2,6-dione (1, 44.4 g, 142 mmol) in MeCN (50 mL) was added to the reaction mixture and stirred at 85° C. for 7 h under N₂ atmosphere. The reaction mixture was diluted with H₂O (500 mL) and extracted with ethyl acetate (500 mL×2). The combined organic layers were washed with brine (400 mL×2), dried over Na₂SO₄, filtered and concentrated under reduced pressure to give a residue, which was purified by prep-HPLC (TFA condition; Instrument: Agela-H1000GC500; Column: I.D. 150 mm*H400 mm Phenomenex Luna C18 15 μm; 100 A; 50.00 grams of sample dissolved in 200 mL of MeCN; Flow rate: 500 mL/min; Mobile phase: MeCN/H₂O; Gradient B %: 30-60% 60 min; 60% 45 min) to afford 3-(5-bromo-3-methyl-1H-indazol-1-yl)-1-(4-methoxybenzyl)piperidine-2,6-dione (3, 12.8 g, 28.9 mmol, 41% yield) as a gray solid.

LCMS (ES+): m/z 444.1 [M+H]⁺

### Step 2: 3-(5-bromo-3-methyl-indazol-1-yl)piperidine-2,6-dione (4)

A mixture of 3-(5-bromo-3-methyl-1H-indazol-1-yl)-1-(4-methoxybenzyl)piperidine-2,6-dione (3, 12.0 g, 27.1 mmol) in TfOH (190 g, 1.27 mol, 112 mL) was degassed and purged with N₂ 3 times, the resulting mixture was stirred at 50° C. for 1 h under N₂ atmosphere. The reaction mixture was cooled to 25° C., poured into ice water (200 mL) and extracted with ethyl acetate (200 mL×2). The combined organic layers were washed with brine (200 mL), dried over Na₂SO₄, filtered and concentrated under reduced pressure to give a residue, which was purified by column chromatography (petroleum ether/ethyl acetate=5/1 to 1/1) to afford 3-(5-bromo-3-methyl-indazol-1-yl)piperidine-2,6-dione (4, 4.00 g, 12.4 mmol, 46% yield) as a light yellow solid.

LCMS (ES+): m/z 322.1 [M+H]⁺

### 3-[3-methyl-5-(4-piperidyl) indazol-1-yl]piperidine-2,6-dione (4)

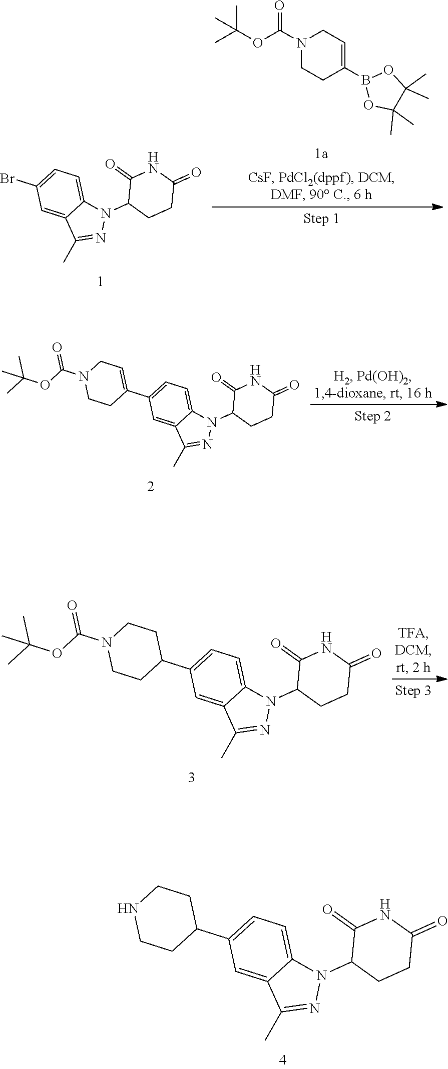 [SMILES: CC(C)(C)OC(=O)N1CC=C(B2OC(C)(C)C(C)(C)O2)CC1 ; Cc1nn(C2CCC(=O)NC2=O)c2ccc(Br)cc12 ; Cc1nn(C2CCC(=O)NC2=O)c2ccc(C3=CCN(C(=O)OC(C)(C)C)CC3)cc12 ; Cc1nn(C2CCC(=O)NC2=O)c2ccc(C3CCN(C(=O)OC(C)(C)C)CC3)cc12 ; Cc1nn(C2CCC(=O)NC2=O)c2ccc(C3CCNCC3)cc12]

### Step 1: tert-butyl 4-[1-(2,6-dioxo-3-piperidyl)-3-methyl-indazol-5-yl]-3,6-dihydro-2H-pyridine-1-carboxylate (2)

Into a 25 mL pressure tube containing a well-stirred solution of 3-(5-bromo-3-methyl-indazol-1-yl)piperidine-2,6-dione (1, 400 mg, 1.24 mmol) and tert-butyl 4-(4,4,5,5-tetramethyl-1,3,2-dioxaborolan-2-yl)-3,6-dihydro-2H-pyridine-1-carboxylate (1a, 579.72 mg, 1.87 mmol) in DMF (6 mL) was added cesium fluoride (471.52 mg, 3.10 mmol, 114.45 μL). The reaction mixture was degassed with nitrogen gas for 10 min. Then, 1,1′-bis(diphenylphosphino)ferrocene]dichloropalladium(II) DCM (304.19 mg, 372.49 μmol) was added and reaction mixture was stirred at 90° C. After 6 h, the reaction mixture was filtered through Celite and washed thoroughly with ethyl acetate (250 mL). The filtrate was concentrated under reduced pressure and purified by flash silica gel column chromatography (60% EtOAc in pet ether) to obtain tert-butyl 4-[1-(2,6-dioxo-3-piperidyl)-3-methyl-indazol-5-yl]-3,6-dihydro-2H-pyridine-1-carboxylate (2, 372 mg, 777.05 μmol, 63% yield) as a brown solid.

LCMS (ES+): m/z 425.6 [M+H]⁺

### Step 2: tert-butyl 4-[1-(2,6-dioxo-3-piperidyl)-3-methyl-indazol-5-yl]piperidine-1-carboxylate (3)

Into a 50 mL single neck round bottom flask containing a well-stirred solution of tert-butyl 4-[1-(2,6-dioxo-3-piperidyl)-3-methyl-indazol-5-yl]-3,6-dihydro-2H-pyridine-1-carboxylate (2, 450 mg, 939.98 μmol) in 1,4-dioxane (3 mL) was added palladium hydroxide 20 wt. % on carbon (462.02 mg, 657.99 μmol). The reaction mixture was stirred under hydrogen atmosphere. After 16 h, the reaction mixture was filtered through Celite, washed with 1,4-dioxane (200 mL) and ethyl acetate (100 mL). The filtrate was concentrated under reduced pressure to afford tert-butyl 4-[1-(2,6-dioxo-3-piperidyl)-3-methyl-indazol-5-yl]piperidine-1-carboxylate (3, 350 mg, 533.40 μmol, 57% yield) as a brown solid. The material was used in the next step without further purification.

LCMS (ES+): m/z 427.7 [M+H]⁺

### Step 3: 3-[3-methyl-5-(4-piperidyl) indazol-1-yl]piperidine-2,6-dione (4)

Into a 50 mL single neck round bottom flask containing a well-stirred solution of tert-butyl 4-[1-(2,6-dioxo-3-piperidyl)-3-methyl-indazol-5-yl]piperidine-1-carboxylate (3, 350 mg, 533.40 μmol) in DCM (2 mL) was added TFA (1.22 g, 10.67 mmol, 821.89 μL). After 2 h the reaction mixture was concentrated and the residue purified by reverse phase prep HPLC [Purification method: Column: XSelect C18 (150×19) mm, 5 micron; Mobile phase A: 0.1% TFA in water; Mobile phase B: MeCN] to afford 3-[3-methyl-5-(4-piperidyl) indazol-1-yl]piperidine-2,6-dione (4, 230 mg, 510.22 μmol, 96% yield, TFA salt) as an off-white solid.

LCMS (ES+): m/z 327.7 [M+H]⁺

### 3-[4-(4-piperidyl)phenyl]piperidine-2,6-dione (6)

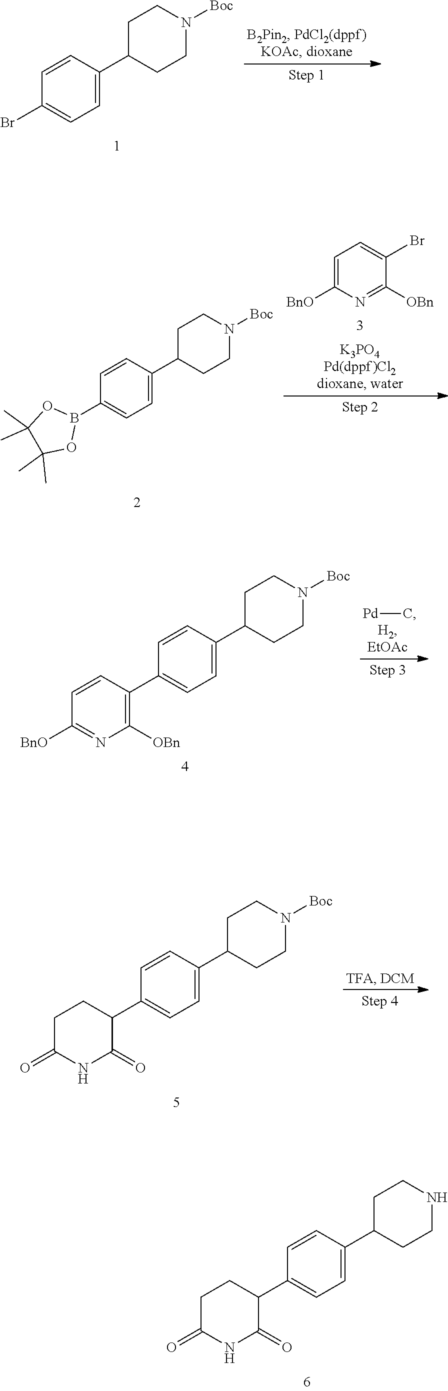

### Step 1: tert-butyl 4-[4-(4,4,5,5-tetramethyl-1,3,2-dioxaborolan-2-yl)phenyl]piperidine-1-carboxylate (2)

To a 500 mL round bottom flask was added a solution of tert-butyl 4-(4-bromophenyl)piperidine-1-carboxylate (1, 10 g, 29.39 mmol) in 1,4-dioxane (100 mL) was added 4,4,5,5-tetramethyl-2-(4,4,5,5-tetramethyl-1,3,2-dioxaborolan-2-yl)-1,3,2-dioxaborolane (11.19 g, 44.08 mmol) followed by the addition of potassium acetate (8.65 g, 88.17 mmol) at room temperature under argon atmosphere. The reaction mixture was degassed with argon for 20 min, after which cyclopentyl(diphenyl)phosphane; dichloromethane; dichloropalladium; iron (2.40 g, 2.94 mmol) was added and the reaction was heated at 100° C. for 6 h. The volatiles were removed under reduced pressure and the residue was extracted with ethyl acetate (200 mL×3) and water (200 mL). The combined organic layers were washed with brine solution (200 mL), dried over anhydrous sodium sulfate, filtered, and concentrated under reduced pressure. The crude product was purified by column chromatography (silica gel 100-200 mesh, 0-30% EtOAc in pet-ether) to afford tert-butyl 4-[4-(4,4,5,5-tetramethyl-1,3,2-dioxaborolan-2-yl)phenyl]piperidine-1-carboxylate (2, 10 g, 24.27 mmol, 83% yield) as a pale yellow solid. LCMS (ES⁺): m/z 332.41 [M−56+H]⁺.

### Step 2: tert-butyl 4-[4-(2,6-dibenzyloxy-3-pyridyl)phenyl]piperidine-1-carboxylate (4)

To a 500 mL round bottom flask was added a solution of tert-butyl 4-[4-(4,4,5,5-tetramethyl-1,3,2-dioxaborolan-2-yl)phenyl]piperidine-1-carboxylate (2, 10 g, 25.82 mmol) in 1,4-dioxane (120 mL) and water (30 mL), followed by the addition of 2,6-dibenzyloxy-3-bromo-pyridine (3, 10.04 g, 27.11 mmol) and potassium phosphate tribasic anhydrous (16.44 g, 77.46 mmol) at room temperature under argon atmosphere. The reaction mixture was degassed with argon for 20 min, after which cyclopentyl(diphenyl)phosphane; dichloropalladium; iron (1.89 g, 2.58 mmol) was added and the reaction was heated at 110° C. for 16 h. The mixture was filtered Celite and washed with ethyl acetate (100 mL×3). The filtrate was washed with water (100 mL) and brine solution (100 mL). The combined organic layers were dried over anhydrous sodium sulfate, filtered, and concentrated under reduced pressure. The crude product was purified by column chromatography (silica gel 230-400 mesh, 0-40% ethyl acetate in pet-ether) and then triturated with pet ether to afford tert-butyl 4-[4-(2,6-dibenzyloxy-3-pyridyl)phenyl]piperidine-1-carboxylate (4, 7 g, 11.57 mmol, 45% yield) as a white color solid. LCMS (ES⁺): m/z 551.43 [M+H]⁺.

### Step 3: tert-butyl 4-[4-(2,6-dioxo-3-piperidyl)phenyl]piperidine-1-carboxylate (5)

A solution of tert-butyl 4-[4-(2,6-dibenzyloxy-3-pyridyl)phenyl]piperidine-1-carboxylate (4, 14 g, 25.42 mmol) in ethyl acetate (420 mL) was added 10% wt. palladium on charcoal (14 g, 25.42 mmol), and the reaction was stirred under hydrogen pressure (70 psi) at room temperature for 16 h. The mixture was filtered through Celite and washed with ethyl acetate (200 mL). The filtrate was concentrated under reduced pressure and the residue was triturated in pentane (100 mL) and diethyl ether (100 mL), dried, and concentrated under reduced pressure to afford tert-butyl 4-[4-(2,6-dioxo-3-piperidyl)phenyl]piperidine-1-carboxylate (5, 8.6 g, 23.05 mmol, 91% yield) as a white solid. LCMS (ES−): m/z 371.23 [M−H]⁻.

### Step 4: 3-[4-(4-piperidyl)phenyl]piperidine-2,6-dione (6)

To a stirred solution of tert-butyl 4-[4-(2,6-dioxo-3-piperidyl)phenyl]piperidine-1-carboxylate (5, 250 mg, 671.22 μmol) in DCM (5 mL) was added TFA (5.92 g, 51.92 mmol, 4 mL) at 0° C. The reaction was stirred for 2 h. The reaction mixture was concentrated and the residue triturated with diethyl ether to obtain 3-[4-(4-piperidyl)phenyl]piperidine-2,6-dione (6, 250 mg, 404.22 μmol, 60% yield, TFA salt) as a brown liquid. LCMS (ES−): m/z 371.23 [M−H]⁻.

### 1-(6-bromo-5-fluoro-1-methyl-indazol-3-yl)hexahydropyrimidine-2,4-dione

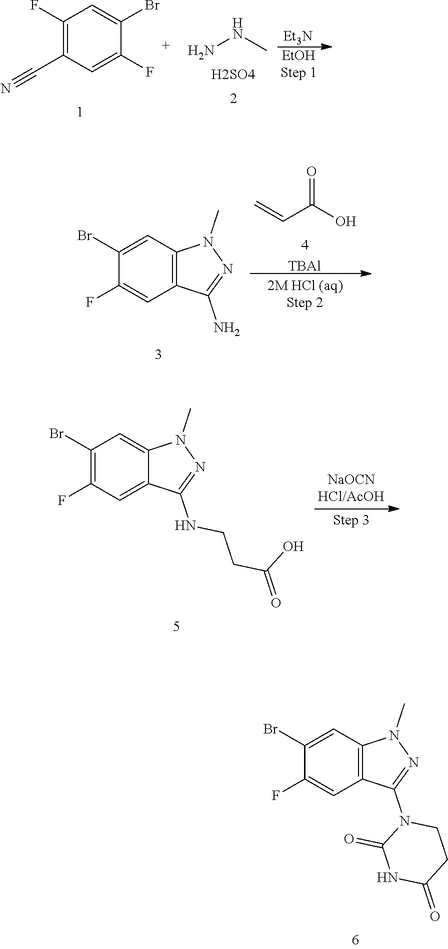

### Step 1: 6-bromo-5-fluoro-1-methyl-indazol-3-amine (3)

To a solution of 4-bromo-2,5-difluoro-benzonitrile (1, 10 g, 45.87 mmol) in EtOH (30 mL) was added methylhydrazine sulfuric acid (2, 19.84 g, 137.62 mmol) and Et₃N (18.57 g, 183.49 mmol, 25.61 mL). The mixture was stirred at 80° C. for 12 h. The mixture was cooled down to 30° C., water (300 mL) was added. The mixture was filtered and the filter cake was washed with water (5 mL×2), and then concentrated at 40° C. under vacuum to afford 6-bromo-5-fluoro-1-methyl-indazol-3-amine (3, 6.5 g, 25.30 mmol, 55% yield) as a yellow solid.

LCMS (ES+): m/z 245 [M+H]⁺

¹H-NMR (400 MHz, DMSO-d₆) δ=7.46 (d, J=8.4 Hz, 1H), 7.06-7.04 (m, 1H), 5.68 (s, 2H), 3.83 (d, J=0.8 Hz, 3H).

### Step 2: 3-[(6-bromo-5-fluoro-1-methyl-indazol-3-yl)amino] propanoic acid (5)

To a solution of 6-bromo-5-fluoro-1-methyl-indazol-3-amine (3, 22 g, 90.14 mmol) and acrylic acid (4, 9.74 g, 135.21 mmol, 9.28 mL) in 2 M aq. HCl (220 mL) was added tetrabutylammonium bromide (2.91 g, 9.01 mmol). The mixture was stirred at 100° C. for 12 h. The reaction mixture was basified with a saturated solution of NaHCO₃ to pH=8. The solution was acidified with acetic acid to pH=5. The white solid precipitate was filtered and washed with water (250 mL), then dried under reduced pressure to afford 3-[(6-bromo-5-fluoro-1-methyl-indazol-3-yl)amino]propanoic acid (5, 28 g, 88.57 mmol, 98% yield) as a white solid. LCMS (ES+): m/z 318.2 [M+H]⁺.

### Step 3: 1-(6-bromo-5-fluoro-1-methyl-indazol-3-yl)hexahydropyrimidine-2,4-dione (6)

To a solution of 3-[(6-bromo-5-fluoro-1-methyl-indazol-3-yl)amino]propanoic acid (5, 26 g, 82.25 mmol) in AcOH (260 mL) was added NaOCN (11.36 g, 164.49 mmol). The mixture was stirred at 60° C. for 16 h. To the mixture was added HCl (260 mL). The mixture was stirred at 60° C. for another 3 h. The reaction mixture was cooled to room temperature and stirred for 1 h, filtered and washed with water (250 mL). The filter cake was dried under vacuum to afford 1-(6-bromo-5-fluoro-1-methyl-indazol-3-yl)hexahydropyrimidine-2,4-dione (6, 18 g, 47.63 mmol, 58% yield) as a white solid. ¹H-NMR (400 MHz, DMSO-d₆) δ=10.59 (s, 1H), 8.16 (d, J=5.6 Hz, 1H), 7.62 (d, J=9.2 Hz, 1H), 4.00 (s, 3H), 3.93-3.90 (m, 2H), 2.77-2.73 (m, 2H).

### 2-(3-((1-(2,6-bis(benzyloxy)pyridin-3-yl)-3-methyl-2-oxo-2,3-dihydro-1H-benzo[d]imidazol-5-yl)amino)phenyl)acetic acid (4)

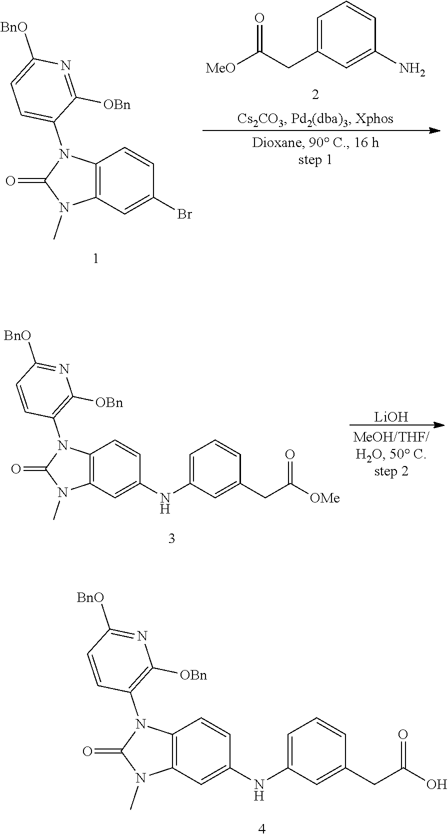 [SMILES: Cn1c(=O)n(-c2ccc(OCc3ccccc3)nc2OCc2ccccc2)c2ccc(Br)cc21 ; COC(=O)Cc1cccc(N)c1 ; COC(=O)Cc1cccc(Nc2ccc3c(c2)n(C)c(=O)n3-c2ccc(OCc3ccccc3)nc2OCc2ccccc2)c1 ; "LiOH" ; Cn1c(=O)n(-c2ccc(OCc3ccccc3)nc2OCc2ccccc2)c2ccc(Nc3cccc(CC(=O)O)c3)cc21]

### Step 1: methyl 2-(3-((1-(2,6-bis(benzyloxy)pyridin-3-yl)-3-methyl-2-oxo-2,3-dihydro-1H-benzo[d]imidazol-5-yl)amino)phenyl)acetate (3)

To a mixture of 1-(2,6-bis(benzyloxy)pyridin-3-yl)-5-bromo-3-methyl-1H-benzo[d]imidazol-2 (3H)-one (1, 784.6 mg, 1.51 mmol) and methyl 2-(3-aminophenyl)acetate (2, 250.0 mg, 3.87 mmol) in Dioxane (8 mL) were added Cs₂CO₃ (986.2 mg, 3.03 mmol) and (1E,4E)-1,5-diphenylpenta-1,4-dien-3-one; palladium (138.59 mg, 151.34 μmol). The mixture was stirred at 90° C. for 16 h. The mixture was filtered and concentrated in vacuum. The residue was purified by column chromatography (SiO₂, Petroleum ether/Ethyl acetate=100/1 to 1/1) to afford methyl 2-(3-((1-(2,6-bis(benzyloxy)pyridin-3-yl)-3-methyl-2-oxo-2,3-dihydro-1H-benzo[d]imidazol-5-yl)amino)phenyl)acetate (3, 0.7 g, 0.99 mmol, 85% yield) as a yellow solid.

LCMS (ESI): m/z 601.1 [M+H]⁺

HNMR (400 MHz, DMSO-d6) δ=8.03 (s, 1H), 7.83-7.77 (m, 1H), 7.48-7.42 (m, 2H), 7.41-7.34 (m, 3H), 7.32-7.23 (m, 5H), 7.12 (s, 1H), 7.00-6.86 (m, 3H), 6.73 (dd, J=2.0, 8.4 Hz, 1H), 6.62 (s, 1H), 6.60-6.58 (m, 1H), 5.46-5.31 (m, 4H), 3.60 (s, 3H), 3.57 (s, 2H), 3.36-3.33 (m, 3H), 3.30-3.29 (m, 1H).

### Step 2: 2-(3-((1-(2,6-bis(benzyloxy)pyridin-3-yl)-3-methyl-2-oxo-2,3-dihydro-1H-benzo[d]imidazol-5-yl)amino)phenyl)acetic acid (4)

A mixture of methyl 2-(3-((1-(2,6-bis(benzyloxy)pyridin-3-yl)-3-methyl-2-oxo-2,3-dihydro-1H-benzo[d]imidazol-5-yl)amino)phenyl)acetate (3, 0.6 g, 1.0 mmol) and LiOH·H₂O (0.36 g, 14.9 mmol) in H₂O (3 mL), MeOH (3 mL) and THF (3 mL) was stirred at 50° C. for 16 h. The mixture was adjusted to pH 5 using 2 N HCl aqueous. The mixture was extracted with ethyl acetate (10 mL). The organic phase was washed with brine (10 mL), dried with anhydrous Na₂SO₄, filtered and concentrated in vacuum. The residue was purified by column chromatography (SiO₂, Petroleum ether/Ethyl acetate=100/1 to 1/1) to afford 2-(3-((1-(2,6-bis(benzyloxy)pyridin-3-yl)-3-methyl-2-oxo-2,3-dihydro-1H-benzo[d]imidazol-5-yl)amino)phenyl)acetic acid (4, 0.3 g, 0.4 mmol, 78% yield) as a yellow solid.

LCMS (ESI): m/z 586.8 [M+H]+

HNMR (400 MHz, DMSO-d6) δ=12.25 (br s, 1H), 8.02 (s, 1H), 7.80 (d, J=8.3 Hz, 1H), 7.47-7.42 (m, 2H), 7.41-7.33 (m, 3H), 7.31-7.25 (m, 5H), 7.12 (t, J=7.8 Hz, 1H), 6.99-6.92 (m, 2H), 6.87 (br d, J=8.1 Hz, 1H), 6.73 (dd, J=1.9, 8.4 Hz, 1H), 6.65-6.61 (m, 1H), 6.60-6.56 (m, 1H), 5.45-5.32 (m, 4H), 3.46 (s, 2H), 3.35-3.33 (m, 3H), 3.29 (br s, 1H).

### 2-[1-[1-(2,6-dioxo-3-piperidyl)-3-methyl-2-oxo-benzimidazol-5-yl]-4-hydroxy-4-piperidyl]acetic acid (5)

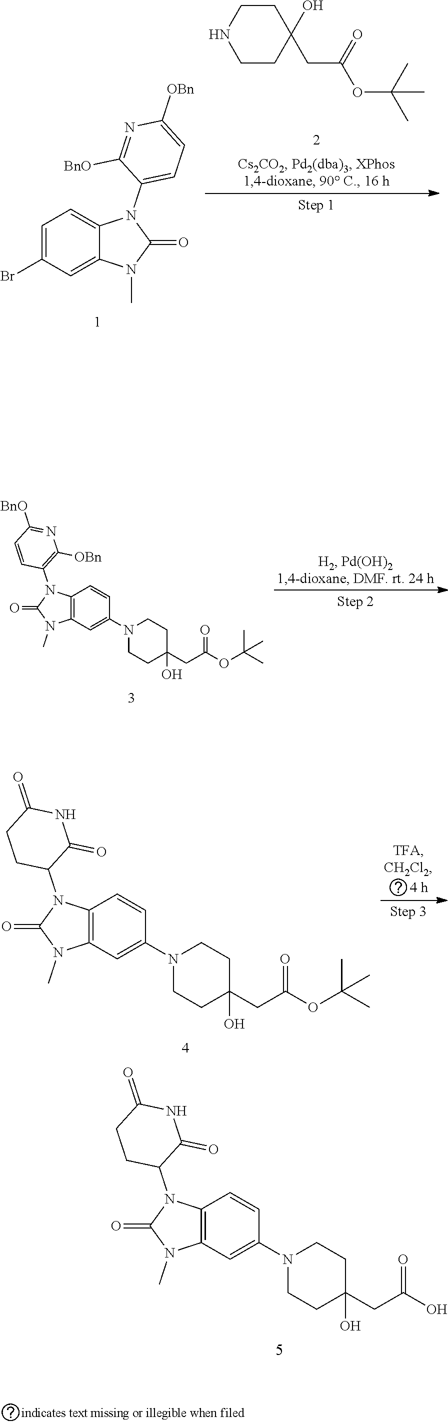 [SMILES: Cn1c(=O)n(-c2ccc(OCc3ccccc3)nc2OCc2ccccc2)c2ccc(Br)cc21 ; CC(C)(C)OC(=O)CC1(O)CCNCC1 ; Cn1c(=O)n(-c2ccc(OCc3ccccc3)nc2OCc2ccccc2)c2ccc(N3CCC(O)(CC(=O)OC(C)(C)C)CC3)cc21 ; Cn1c(=O)n(C2CCC(=O)NC2=O)c2ccc(N3CCC(O)(CC(=O)OC(C)(C)C)CC3)cc21 ; Cn1c(=O)n(C2CCC(=O)NC2=O)c2ccc(N3CCC(O)(CC(=O)O)CC3)cc21]

### Step 1: tert-butyl 2-[1-[1-(2,6-dibenzyloxy-3-pyridyl)-3-methyl-2-oxo-benzimidazol-5-yl]-4-hydroxy-4-piperidyl]acetate (3)

Into a 50 mL pressure tube containing a well-stirred solution of 5-bromo-1-(2,6-dibenzyloxy-3-pyridyl)-3-methyl-benzimidazol-2-one (1, 700 mg, 1.33 mmol) and tert-butyl 2-(4-hydroxy-4-piperidyl)acetate (2, 451.58 mg, 1.99 mmol) in anhydrous 1,4-dioxane (7 mL) was added cesium carbonate (1.30 g, 3.99 mmol). The mixture was degassed with nitrogen for 15 min. Then, tris(dibenzylideneacetone)dipalladium(0) (145.98 mg, 159.42 μmol) and XPhos (126.66 mg, 265.69 μmol) were added. The vial was sealed and heated at 90° C. After 16 h, the reaction mixture was filtered through Celite and washed with EtOAc (50 mL). The filtrate was concentrated under reduced pressure and the residue was purified by flash silica gel column chromatography (90% EtOAc in petroleum ether) to afford tert-butyl 2-[1-[1-(2,6-dibenzyloxy-3-pyridyl)-3-methyl-2-oxo-benzimidazol-5-yl]-4-hydroxy-4-piperidyl]acetate (3, 600 mg, 901.62 μmol, 68% yield) as pale yellow solid. LCMS (ES+): m/z 651.3 [M+H]⁺.

### Step 2: tert-butyl 2-[1-[1-(2,6-dioxo-3-piperidyl)-3-methyl-2-oxo-benzimidazol-5-yl]-4-hydroxy-4-piperidyl]acetate (4)

Into a 50 mL single neck round bottom flask containing a well-stirred solution of tert-butyl 2-[1-[1-(2,6-dibenzyloxy-3-pyridyl)-3-methyl-2-oxo-benzimidazol-5-yl]-4-hydroxy-4-piperidyl]acetate (3, 600 mg, 894.34 μmol) in anhydrous 1,4-dioxane (15 mL) was added palladium hydroxide on carbon (20 wt. %, 50% water) (356.33 mg, 507.46 μmol). The suspension was stirred under an atmosphere of hydrogen for 20 h. The reaction mixture was filtered through Celite and concentrated under reduced pressure. The residue was purified by reverse phase prep HPLC [Purification method: Column: X-Bridge C18 (150×19) 5 micron; Mobile phase A: 0.1% TFA in water; Mobile phase B: MeCN] to afford tert-butyl 2-[1-[1-(2,6-dioxo-3-piperidyl)-3-methyl-2-oxo-benzimidazol-5-yl]-4-hydroxy-4-piperidyl]acetate (4, 400 mg, 681.40 μmol, 76% yield, TFA salt) as a white solid. LCMS (ES+): m/z 473.2 [M+H]⁺.

### Step 3: 2-[1-[1-(2,6-dioxo-3-piperidyl)-3-methyl-2-oxo-benzimidazol-5-yl]-4-hydroxy-4-piperidyl]acetic acid (5)

Into a 50 mL single neck round bottom flask containing a well-stirred solution of tert-butyl 2-[1-[1-(2,6-dioxo-3-piperidyl)-3-methyl-2-oxo-benzimidazol-5-yl]-4-hydroxy-4-piperidyl]acetate (4, 380 mg, 788.09 μmol) in anhydrous DCM (5 mL) was added TFA (1.78 g, 15.58 mmol, 1.2 mL). After 4 h, the solvent was removed under reduced pressure and the residue was triturated with diethyl ether (10 mL), filtered and dried to afford 2-[1-[1-(2,6-dioxo-3-piperidyl)-3-methyl-2-oxo-benzimidazol-5-yl]-4-hydroxy-4-piperidyl]acetic acid (5, 400 mg, 650.24 μmol, 83% yield, TFA salt) as an off-white solid. LCMS (ES+): m/z 417.2 [M+H]⁺.

### 2-(1-(1-(2,6-bis(benzyloxy)pyridin-3-yl)-3-methyl-2-oxo-2,3-dihydro-1H-benzo[d]imidazol-5-yl)-1H-indol-5-yl)acetic acid (6)

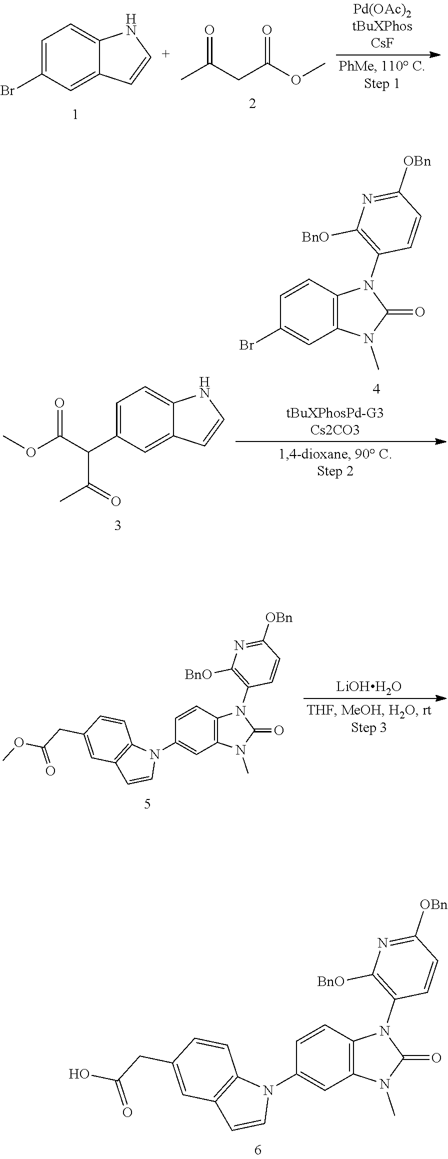

### Step 1: methyl 2-(1H-indol-5-yl)-3-oxo-butanoate (3)

A mixture of 5-bromo-1H-indole (1, 10 g, 51.01 mmol), methyl 3-oxobutanoate (2, 5.92 g, 51.01 mmol, 5.48 mL), tert-Butyl XPhos (2.17 g, 5.10 mmol), cesium fluoride (30.99 g, 204.04 mmol, 7.52 mL) and diacetoxypalladium (572.60 mg, 2.55 mmol) in Toluene (100 mL) was degassed and purged with N₂ three times, and then the mixture was stirred at 110° C. for 16 h under N₂ atmosphere. The reaction mixture was poured into water (200 mL) and extracted with ethyl acetate (2*200 mL). Organic phases were combined and washed with brine (200 mL), dried over anhydrous sodium sulfate, filtered and concentrated in vacuum. The residue was purified by flash silica gel chromatography (Biotage®; 120 g SepaFlash® Silica Flash Column, Eluent of 0-50% Ethylacetate/Petroleum ether gradient @ 100 mL/min) to afford methyl 2-(1H-indol-5-yl)-3-oxo-butanoate (3, 2.6 g, 10.08 mmol, 20% yield) as yellow oil. LCMS (ES+): m/z 232.0[M+H]⁺

### Step 2: methyl 2-(1-(1-(2,6-bis(benzyloxy)pyridin-3-yl)-3-methyl-2-oxo-2,3-dihydro-1H-benzo[d]imidazol-5-yl)-1H-indol-5-yl)acetate (5)

To a mixture of 1-(2,6-bis(benzyloxy)pyridin-3-yl)-5-bromo-3-methyl-1H-benzo[d]imidazol-2 (3H)-one (4, 1 g, 1.94 mmol), methyl 2-(1H-indol-5-yl)-3-oxobutanoate (3, 500.00 mg, 2.16 mmol), tBuXPhos-Pd-G3 (153.83 mg, 193.65 μmol) and Cs₂CO₃ (1.89 g, 5.81 mmol) was added dioxane (10 mL). The mixture was stirred at 90° C. for 16 h under N₂. The reaction mixture was concentrated under vacuum. The residue was purified by flash silica gel chromatography (flow: 50 mL/min; gradient: 0-100% ethylacetate in petroleum ether; ISCO®; 40 g SepaFlash®Silica FlashColumn; ethylacetate/petroleumether=1/0 Rf=0.6) to afford methyl 2-(1-(1-(2,6-bis(benzyloxy)pyridin-3-yl)-3-methyl-2-oxo-2,3-dihydro-1H-benzo[d]imidazol-5-yl)-1H-indol-5-yl)acetate (5, 860 mg, 1.00 mmol, 52% yield) as yellow solid.

LCMS: (ES+): m/z 625.2 [M+H]⁺

### Step 3: 2-(1-(1-(2,6-bis(benzyloxy)pyridin-3-yl)-3-methyl-2-oxo-2,3-dihydro-1H-benzo[d]imidazol-5-yl)-1H-indol-5-yl)acetic acid (6)

To a solution of methyl 2-[1-[1-(2,6-dibenzyloxy-3-pyridyl)-3-methyl-2-oxo-benzimidazol-5-yl]indol-5-yl]acetate (5, 860 mg, 1.38 mmol) in THF (3 mL) and Methanol (3 mL) was added a solution of LiOH·H2O (173.31 mg, 4.13 mmol) in Water (3 mL). After 2 h the reaction was quenched with 1N HCl and adjusted to pH to 6-7, then concentrated. The residue was purified by reversed phase HPLC (FA) and then lyophilization to afford 2-(1-(1-(2,6-bis(benzyloxy)pyridin-3-yl)-3-methyl-2-oxo-2,3-dihydro-1H-benzo[d]imidazol-5-yl)-1H-indol-5-yl)acetic acid (6, 320 mg, 513.55 μmol, 37% yield) as yellow solid.

LCMS: (ES+): m/z 611.1 [M+H]⁺

### 3-(1-methyl-2-oxo-6,7,8,9-tetrahydroimidazo[4,5-f]quinolin-3-yl)piperidine-2,6-dione (7)

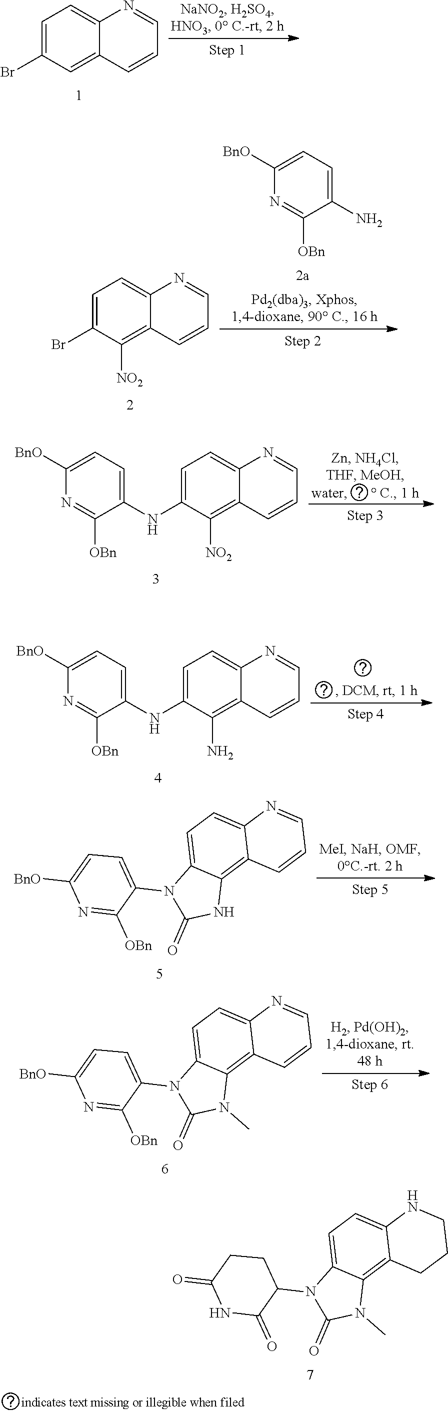

### Step 1: 6-bromo-5-nitro-quinoline (2)

Into a 500 mL round bottom flask containing a well-stirred solution of 6-bromoquinoline (1, 10 g, 48.06 mmol, 6.49 mL) in sulfuric acid (50 mL) were added sodium nitrite (134.98 mg, 1.96 mmol) and nitric acid (95.85 μmol, 4 mL) at 0° C. The mixture was stirred at 0° C. for 45 min and 1 h at ambient temperature. The reaction was quenched with ice water (100 mL) and the yellow solid was filtered, washed with water and dried under reduced pressure to afford 6-bromo-5-nitro-quinoline (2, 8.89 g, 34.78 mmol, 72% yield) as a yellow solid.

LCMS (ES+): m/z 253.0 [M+H]⁺

### Step 2: N-(2,6-dibenzyloxy-3-pyridyl)-5-nitro-quinolin-6-amine (3)

In a 250 mL pressure tube containing a well-stirred solution of 6-bromo-5-nitro-quinoline (2, 7 g, 27.49 mmol) in 1,4-dioxane (70 mL), was added 2,6-dibenzyloxypyridin-3-amine (2a, 11.41 g. 35.73 mmol) followed by cesium carbonate (26.87 g, 82.46 mmol) and the reaction mixture was degassed by bubbling nitrogen gas for 10 min. Then, XPhos (1.31 g, 2.75 mmol) and tris(dibenzylideneacetone)dipalladium(0) (2.52 g, 2.75 mmol) were added and stirred at 90° C. After 16 h, the reaction mixture was filtered through Celite and washed with ethyl acetate (250 mL). The filtrate was concentrated under reduced pressure and purified by flash silica gel (230-400 mesh) column chromatography (40% Ethyl acetate in pet ether) to afford N-(2,6-dibenzyloxy-3-pyridyl)-5-nitro-quinolin-6-amine (3, 11.12 g, 15.74 mmol, 57% yield, 68% purity) as reddish brown gummy liquid.

LCMS (ES+): m/z 479.0 [M+H]⁺

### Step 3: N6-(2,6-dibenzyloxy-3-pyridyl)quinoline-5,6-diamine (4)

Into a 500 mL three neck round bottom flask containing a well-stirred solution of N-(2,6-dibenzyloxy-3-pyridyl)-5-nitro-quinolin-6-amine (3, 11.12 g, 15.57 mmol) in THF (90 mL) and methanol (70 mL) and water (30 mL) was added Zinc Powder (325 mesh High Grade Material) (5.09 g, 77.85 mmol, 712.99 μL) and Ammonium Chloride (4.16 g, 77.85 mmol, 2.72 mL). The reaction mixture was refluxed at 80° C. for 1 h. The reaction mixture was filtered through Celite, washing with ethyl acetate (500 mL) and the filtrate concentrated. The crude was washed with water (200 mL) and extracted with ethyl acetate (2×200 mL). The organic layer was dried over sodium sulfate, filtered and concentrated under reduced pressure to afford N6-(2,6-dibenzyloxy-3-pyridyl)quinoline-5,6-diamine (4, 8 g, 15.52 mmol, 100% yield) as a brown colored thick mass.

LCMS (ES+): m/z 449.2 [M+H]⁺

### Step 4: 3-(2,6-dibenzyloxy-3-pyridyl)-1H-imidazo[4,5-f]quinolin-2-one (5)

Into a 500 mL three-neck round-bottom flask containing a well-stirred solution of N6-(2,6-dibenzyloxy-3-pyridyl)quinoline-5,6-diamine (4, 8 g, 15.52 mmol) in anhydrous DCM (150 mL) was added triphosgene (9.21 g, 31.04 mmol) and stirred for 5 min. Then, pyridine (6.14 g, 77.59 mmol, 6.28 mL) in anhydrous DCM (25 mL) was added at 0° C. The reaction mixture was stirred at room temperature for 2 h. The reaction was quenched with cold water at 0° C. and extracted with 20% methanol in DCM (2×300 mL). The combined organic layer was dried over anhydrous Na₂SO₄, filtered, concentrated and purified by flash silica gel (230-400 mesh) column chromatography (90% ethyl acetate in pet ether) to afford 3-(2,6-dibenzyloxy-3-pyridyl)-1H-imidazo[4,5-f]quinolin-2-one (5, 7.3 g, 14.92 mmol, 96% yield) as a brown solid.

LCMS (ES+): m/z 475.2 [M+H]⁺

### Step 5: 3-(2,6-dibenzyloxy-3-pyridyl)-1-methyl-imidazo[4,5-f]quinolin-2-one (6)

Into a 100 mL single neck round bottom flask containing a well-stirred solution of 3-(2,6-dibenzyloxy-3-pyridyl)-1H-imidazo[4,5-f]quinolin-2-one (5, 7.3 g, 14.92 mmol) in DMF (100 mL) was added sodium hydride (60% in mineral oil) (857.68 mg, 22.38 mmol) at 0° C. The reaction mixture was stirred at 25° C. for 30 min. The reaction mixture was cooled to 0° C. and Iodomethane (4.24 g, 29.85 mmol, 1.86 mL) was added and stirred at room temperature for 2 h. The reaction mixture was quenched with ice water (200 mL) and the precipitate was filtered, washed with water and dried under vacuum to afford 3-(2,6-dibenzyloxy-3-pyridyl)-1-methyl-imidazo[4,5-f]quinolin-2-one (6, 7.5 g, 14.10 mmol, 95% yield) as a brown color solid.

LCMS (ES+): m/z 489.2 [M+H]⁺

### Step 6: 3-(1-methyl-2-oxo-6,7,8,9-tetrahydroimidazo[4,5-f]quinolin-3-yl)piperidine-2,6-dione (7)

Into a 100 mL single neck round bottom flask containing a well-stirred solution of 3-(2,6-dibenzyloxy-3-pyridyl)-1-methyl-imidazo[4,5-f]quinolin-2-one (6, 2 g, 3.73 mmol) in anhydrous 1,4-dioxane (20 mL) was added 20 wt. % palladium hydroxide on carbon (2.35 g, 3.35 mmol). The suspension was stirred for 72 h under hydrogen atmosphere. The reaction mixture was filtered and washed with THF (100 mL) and DMF (100 mL). The filtrate was concentrated and purified by reverse phase column chromatography [Silicycle C18 column; Mobile phase A: 0.1% formic acid in water; Mobile phase B: MeCN] to afford 3-(1-methyl-2-oxo-6,7,8,9-tetrahydroimidazo[4,5-f]quinolin-3-yl)piperidine-2,6-dione (7, 800 mg, 1.99 mmol, 53% yield, formic acid salt) as a light pink color solid.

LCMS (ES+): m/z 315.1 [M+H]⁺

### (R)-2-((1-(1-(2,6-bis(benzyloxy)pyridin-3-yl)-3-methyl-2-oxo-2,3-dihydro-1H-benzo[d]imidazol-4-yl)piperidin-3-yl)oxy)acetic acid (8A) & (S)-2-((1-(1-(2,6-bis(benzyloxy)pyridin-3-yl)-3-methyl-2-oxo-2,3-dihydro-1H-benzo[d]imidazol-4-yl)piperidin-3-yl)oxy)acetic acid (8B)

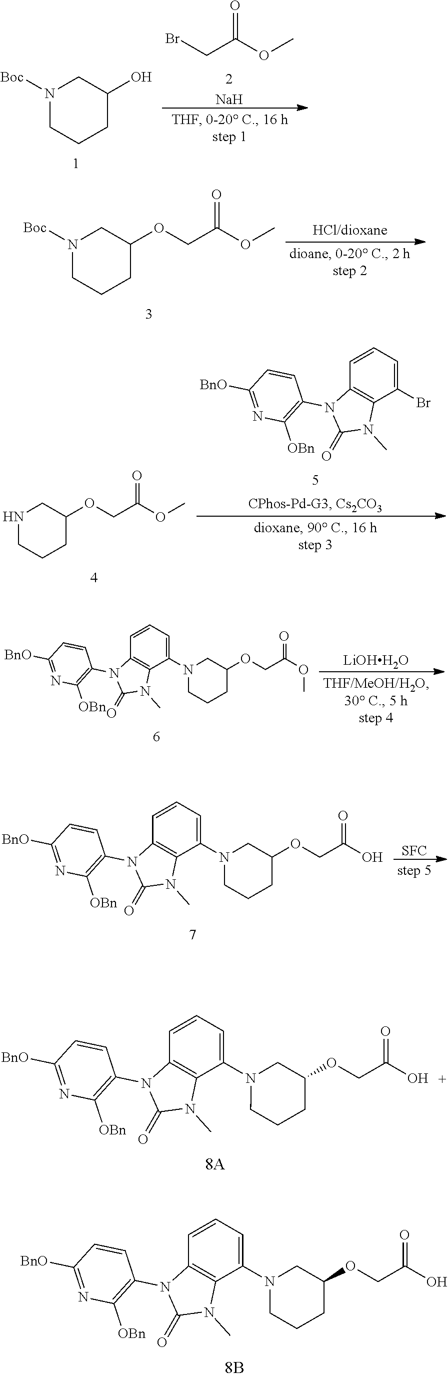

### Step 1: tert-butyl 3-(2-methoxy-2-oxoethoxy)piperidine-1-carboxylate (3)

To a solution of tert-butyl 3-hydroxypiperidine-1-carboxylate (1, 10 g, 49.69 mmol) and NaH (1.99 g, 49.69 mmol, 60% purity) in THF (200 mL) at 0° C. was added methyl 2-bromoacetate (2, 7.60 g, 49.69 mmol, 4.58 mL) drop wise. The resulting mixture was stirred at 20° C. for 16 h. The reaction mixture was quenched by addition of H₂O (200 mL) at 0° C., and then extracted with ethyl acetate (200 mL×2). Organic phases were combined and washed with brine (300 mL), dried over anhydrous Na₂SO₄, filtered and concentrated. The residue was purified by column chromatography (SiO₂, Petroleum ether/Ethyl acetate=1/0 to 5/1; Petroleum ether/Ethyl acetate=2/1, R_{f}=0.6) to afford tert-butyl 3-(2-methoxy-2-oxoethoxy)piperidine-1-carboxylate (3, 6.5 g, 23.54 mmol, 47% yield) as a colorless oil.

1H NMR (400 MHz, d-CHLOROFORM) δ 4.24-4.12 (m, 2H), 3.77 (s, 3H), 3.62 (td, J=4.8, 13.2 Hz, 1H), 3.43 (tt, J=4.0, 8.0 Hz, 1H), 3.08 (d, J=9.6 Hz, 2H), 2.00 (d, J=5.6 Hz, 1H), 1.82-1.74 (m, 1H), 1.62-1.55 (m, 1H), 1.51-1.38 (m, 11H).

### Step 2: methyl 2-(piperidin-3-yloxy)acetate (4)

To a solution of tert-butyl 3-(2-methoxy-2-oxoethoxy)piperidine-1-carboxylate (3, 4.2 g, 15.37 mmol) in dioxane (5 mL) was added HCl/dioxane (4 M, 30 mL) at 0° C. The mixture was stirred at 20° C. for 2 h. The reaction mixture was concentrated under reduced pressure to afford methyl 2-(piperidin-3-yloxy)acetate (4, 3.5 g, 20.21 mmol) as colorless oil. The crude product was used in the next step without purification.

### Step 3: methyl 2-((1-(1-(2,6-bis(benzyloxy)pyridin-3-yl)-3-methyl-2-oxo-2,3-dihydro-1H-benzo[d]imidazol-4-yl)piperidin-3-yl)oxy)acetate (6)

A mixture of 1-(2,6-bis(benzyloxy)pyridin-3-yl)-4-bromo-3-methyl-1H-benzo[d]imidazol-2 (3H)-one (5, 500 mg, 968.27 μmol) and methyl 2-(piperidin-3-yloxy)acetate (4, 503.14 mg, 2.90 mmol) in dioxane (10 mL) were added CS₂CO₃ (1.58 g, 4.84 mmol) and Pd-PEPPSI-IHeptCl (94.21 mg, 96.83 μmol). The mixture was degassed and purged with N₂ 3 times, and then the mixture was stirred at 90° C. for 16 h under N₂ atmosphere. The reaction mixture was filtered and the filtrate was concentrated under reduced pressure. The residue was purified by flash silica gel chromatography (60 mL/min, Eluent of 0-50% ethyl acetate/petroleum ether gradient, Column: ISCO; 10 g SepaFlash Silica Flash Column; Petroleum ether/Ethyl acetate=5/1, R_{f}=0.5) and purified by prep-HPLC (flow: 60 mL/min; gradient: from 62-92% water (0.1% TFA)-ACN over 11 min; column: Phenomenex luna C18 150×40 mm×15 um) to afford methyl 2-((1-(1-(2,6-bis(benzyloxy)pyridin-3-yl)-3-methyl-2-oxo-2,3-dihydro-1H-benzo[d]imidazol-4-yl)piperidin-3-yl)oxy)acetate (6, 430 mg, 607.54 μmol, 37% yield) as a white solid.

LCMS (ESI): m/z 609.3 [M+H]⁺

¹H NMR (400 MHz, d₆-DMSO) δ 7.79 (d, J=8.0 Hz, 1H), 7.46-7.43 (m, 2H), 7.41-7.34 (m, 3H), 7.29-7.25 (m, 5H), 6.97-6.91 (m, 2H), 6.64-6.59 (m, 1H), 6.47-6.40 (m, 1H), 5.41-5.34 (m, 4H), 4.23 (s, 2H), 3.71 (d, J=6.8 Hz, 3H), 3.50-3.39 (m, 1H), 3.33 (s, 3H), 3.04 (dd, J=2.4, 5.6 Hz, 1H), 2.65-2.56 (m, 1H), 2.28-2.09 (m, 1H), 1.83-1.60 (m, 2H), 1.56-1.39 (m, 1H), 1.33-1.22 (m, 1H), 0.90-0.77 (m, 1H)

### Step 4: 2-((1-(1-(2,6-bis(benzyloxy)pyridin-3-yl)-3-methyl-2-oxo-2,3-dihydro-1H-benzo[d]imidazol-4-yl)piperidin-3-yl)oxy)acetic acid (7)

To a solution of methyl 2-((1-(1-(2,6-bis(benzyloxy)pyridin-3-yl)-3-methyl-2-oxo-2,3-dihydro-1H-benzo[d]imidazol-4-yl)piperidin-3-yl)oxy)acetate (6, 380 mg, 624.30 μmol) in THF (3 mL), MeOH (3 mL) and H₂O (3 mL) was LiOH·H₂O (130.99 mg, 3.12 mmol). The mixture was stirred at 30° C. for 5 h. The reaction mixture was adjusted pH to 5 with 1 N HCl aqueous. Then the reaction mixture was diluted with water (10 mL) and extracted with ethyl acetate (20 mL×3). The combined organic layers were washed with brine (30 mL), dried over Na₂SO₄, filtered and concentrated under reduced pressure to afford 2-((1-(1-(2,6-bis(benzyloxy)pyridin-3-yl)-3-methyl-2-oxo-2,3-dihydro-1H-benzo[d]imidazol-4-yl)piperidin-3-yl)oxy)acetic acid (7,350 mg, 582.69 μmol, 93% yield) as a yellow solid. The crude product was used to next step without purification.

LCMS (ESI): m/z 595.0 [M+H]⁺

### Step 5: (R)-2-((1-(1-(2,6-bis(benzyloxy)pyridin-3-yl)-3-methyl-2-oxo-2,3-dihydro-1H-benzo[d]imidazol-4-yl)piperidin-3-yl)oxy)acetic acid (8A) and (S)-2-((1-(1-(2,6-bis(benzyloxy)pyridin-3-yl)-3-methyl-2-oxo-2,3-dihydro-1H-benzo[d]imidazol-4-yl)piperidin-3-yl)oxy)acetic acid (8B)

The 2-((1-(1-(2,6-bis(benzyloxy)pyridin-3-yl)-3-methyl-2-oxo-2,3-dihydro-1H-benzo[d]imidazol-4-yl)piperidin-3-yl)oxy)acetic acid (7, 320 mg, 538.13 μmol) was purified by SFC (Sample preparation: Add acn 60 ml into sample Instrument: Waters 80Q Mobile Phase: 70% MeOH+ACN (0.1% NH₃H₂O) in Supercritical CO₂ Flow Rate: 80 g/min Cycle Time: 7.1 min, total time: 120 min Single injection volume: 4.0 ml Back Pressure: 100 bar to keep the CO₂ in Supercritical flow) to afford (R)-2-((1-(1-(2,6-bis(benzyloxy)pyridin-3-yl)-3-methyl-2-oxo-2,3-dihydro-1H-benzo[d]imidazol-4-yl)piperidin-3-yl)oxy)acetic acid (8A, 145 mg, 241.40 μmol) as a white solid and (S)-2-((1-(1-(2,6-bis(benzyloxy)pyridin-3-yl)-3-methyl-2-oxo-2,3-dihydro-1H-benzo[d]imidazol-4-yl)piperidin-3-yl)oxy)acetic acid (8A, 190 mg, 316.32 μmol) as a white solid. (8A: peak 1 showed on SFC, R₁=3.970 min), (8B: peak 2 showed on SFC, R₁=6.475 min) LCMS (ESI): m/z 593.5 [M+H]⁺

### 8A:

¹H NMR (400 MHz, d₆-DMSO) δ 7.79 (dd, J=4.0, 8.4 Hz, 1H), 7.46-7.32 (m, 5H), 7.29-7.22 (m, 5H), 6.92 (d, J=4.4 Hz, 2H), 6.60 (d, J=8.4 Hz, 1H), 6.45-6.38 (m, 1H), 5.41-5.32 (m, 4H), 3.87 (s, 2H), 3.64 (s, 4H), 3.44-3.40 (m, 1H), 3.06-2.99 (m, 1H), 2.64-2.52 (m, 1H), 2.44 (d, J=1.6 Hz, 1H), 2.22-2.08 (m, 1H), 1.81-1.53 (m, 2H), 1.27-1.10 (m, 1H)

### 8B:

¹H NMR (400 MHz, d₆-DMSO) δ 7.79 (dd, J=4.0, 8.4 Hz, 1H), 7.46-7.31 (m, 5H), 7.29-7.21 (m, 5H), 6.92 (d, J=4.4 Hz, 2H), 6.60 (d, J=8.4 Hz, 1H), 6.43-6.37 (m, 1H), 5.42-5.32 (m, 4H), 3.83 (s, 2H), 3.65 (s, 4H), 3.46-3.39 (m, 1H), 3.08-2.98 (m, 1H), 2.64-2.52 (m, 1H), 2.44-2.35 (m, 1H), 2.22-2.08 (m, 1H), 1.80-1.57 (m, 2H), 1.21-1.12 (m, 1H)

### tert-butyl 2-[(3S,4S)-1-[1-(2,6-dioxo-3-piperidyl)-3-methyl-2-oxo-benzimidazol-5-yl]-3-methyl-4-piperidyl]acetate (10)

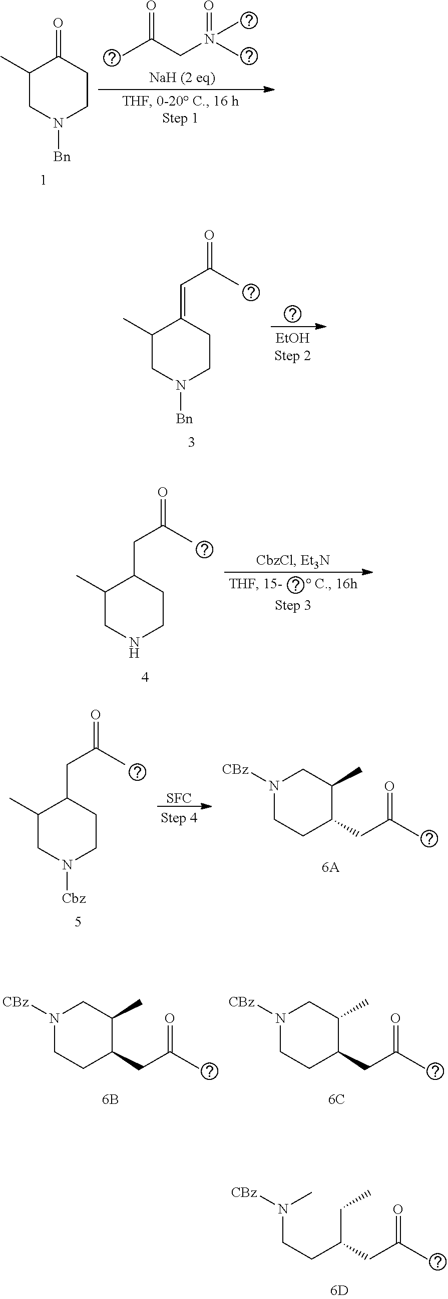

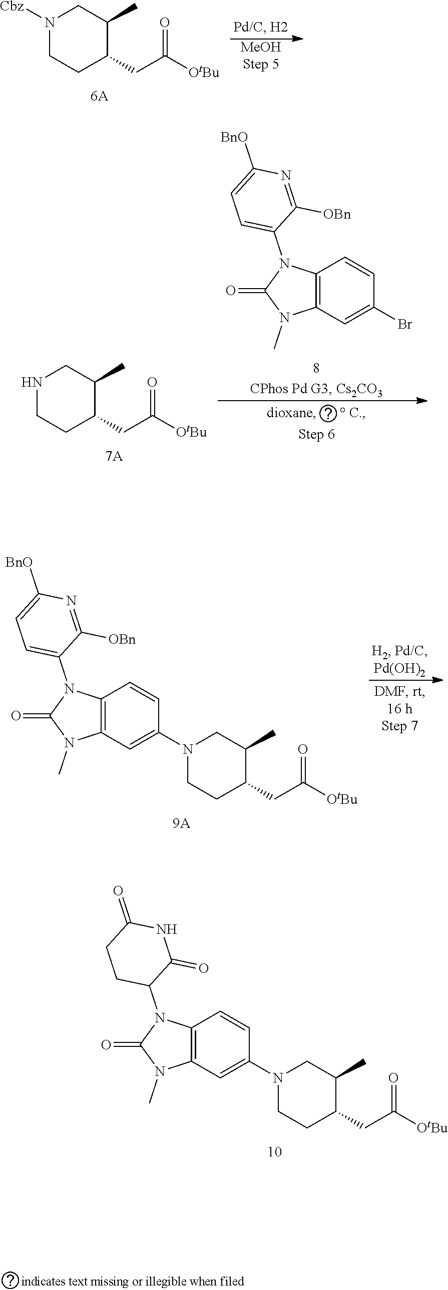 [SMILES: C[C@@H]1CN(C(=O)OCc2ccccc2)CC[C@H]1CC(=O)OC(C)(C)C ; "MeOHStep 5" ; Cn1c(=O)n(-c2ccc(OCc3ccccc3)nc2OCc2ccccc2)c2ccc(Br)cc21 ; C[C@@H]1CNCC[C@H]1CC(=O)OC(C)(C)C ; C[C@@H]1CN(c2ccc3c(c2)n(C)c(=O)n3-c2ccc(OCc3ccccc3)nc2OCc2ccccc2)CC[C@H]1CC(=O)OC(C)(C)C ; C[C@@H]1CN(c2ccc3c(c2)n(C)c(=O)n3C2CCC(=O)NC2=O)CC[C@H]1CC(=O)OC(C)(C)C]

### Step 1: tert-butyl (2E)-2-(1-benzyl-3-methyl-4-piperidylidene)acetate (3)

To a solution of NaH (7.87 g, 196.77 mmol, 60% purity) in THF (200 mL) was added tert-butyl 2-diethoxyphosphorylacetate (2, 37.23 g, 147.58 mmol, 34.79 mL) at 0° C. The mixture was stirred at 20° C. for 0.5 h. Then a solution of 1-benzyl-3-methyl-piperidin-4-one (1, 20 g, 98.39 mmol) in THF (50 mL) was added at 0° C. The mixture was stirred at 20° C. for 16 h under N₂ atmosphere. The reaction mixture (PH>7) was diluted with EtOAc (500 mL) and water (300 mL). The layers were separated and the aqueous phase was extracted with EtOAc (3×250 mL). Combined extracts were washed with brine (250 mL), dried over Na₂SO₄, filtered and concentrated under reduced pressure. The residue purified by column chromatography (SiO₂, Petroleum ether/Ethyl acetate=1/0 to 3/1, Petroleum ether/Ethyl acetate=5:1, RF=0.7) to afford tert-butyl (2E)-2-(1-benzyl-3-methyl-4-piperidylidene)acetate (3, 22 g, 72.99 mmol, 74% yield) as a colorless oil.

1H NMR (400 MHz, DMSO-D) δ (ppm)=7.37-7.22 (m, 5H), 5.52-5.42 (m, 1H), 3.53-3.42 (m, 2H), 2.96-2.82 (m, 1H), 2.70-2.57 (m, 2H), 2.28-2.18 (m, 1H), 2.04-1.94 (m, 1H), 1.45-1.30 (m, 9H), 1.21-0.95 (m, 3H)

### Step 2: tert-butyl 2-(3-methyl-4-piperidyl)acetate (4)

To a solution of tert-butyl (2E)-2-(1-benzyl-3-methyl-4-piperidylidene)acetate (3, 22 g, 72.99 mmol) in Methanol (220 mL) was added Pd/C (2 g, 10% purity) and Pd(OH)₂/C (2 g, 10% purity). The mixture was stirred at 20° C. for 16 h under H2 (15 Psi). The reaction mixture was filtered and concentrated under reduced pressure. The crude product tert-butyl 2-(3-methyl-4-piperidyl)acetate (4, 15 g, 70.32 mmol, 96% yield) was used in the next step without further purification.

1H NMR (400 MHz, CD3SOCD3, 0 K) δ (ppm)=2.90-2.73 (m, 2H), 2.60-2.57 (m, 1H), 2.46-2.34 (m, 2H), 2.11 (br s, 2H), 1.96-1.87 (m, 1H), 1.67-1.58 (m, 1H), 1.57-1.49 (m, 1H), 1.39 (d, J=2.0 Hz, 9H), 1.30-1.23 (m, 1H), 1.21-1.03 (m, 1H), 0.87-0.74 (m, 3H)

### Step 3: benzyl 4-(2-(tert-butoxy)-2-oxoethyl)-3-methylpiperidine-1-carboxylate (5)

To a solution of tert-butyl 2-(3-methyl-4-piperidyl)acetate (4, 7 g, 32.82 mmol) in Water (70 mL) was added benzyl carbonochloridate (11.20 g, 65.63 mmol) and Na₂CO₃ (8.70 g, 82.04 mmol) at 0° C. The mixture was stirred at 20° C. for 16 h. The reaction mixture was filtered and concentrated under reduced pressure. The residue purified by column chromatography (SiO₂, Petroleum ether/Ethyl acetate=1/0 to 3/1) to afford benzyl 4-(2-(tert-butoxy)-2-oxoethyl)-3-methylpiperidine-1-carboxylate (5), which was taken directly to SFC separation.

### Step 4: benzyl 4-(2-(tert-butoxy)-2-oxoethyl)-3-methylpiperidine-1-carboxylate isomers (6A, 6B, 6C, 6D)

benzyl 4-(2-(tert-butoxy)-2-oxoethyl)-3-methylpiperidine-1-carboxylate (5) was purified by SFC (Sample preparation: Add ETOH 30 mL into sample Instrument: Waters 80Q Mobile Phase: 20% MeOH (0.1% NH₃H2O) in Supercritical CO₂ Flow Rate: 55 g/min Cycle Time: 5.3 min, total time: 40 min Single injection volume: 4.0 ml Back Pressure: 100 bar to keep the CO₂ in Supercritical flow) to obtain benzyl (3R,4S)-4-(2-tert-butoxy-2-oxo-ethyl)-3-methyl-piperidine-1-carboxylate (6D, 1.3 g, 3.74 mmol, 11% yield). After purified by SFC(Sample preparation: Add ETOH 30 ml into sample Instrument: Waters 80Q Mobile Phase: 15% MeOH (0.1% NH₃H2O) in Supercritical CO₂ Flow Rate: 50 g/min Cycle Time: 3.2 min, total time: 75 min Single injection volume: 1.0 ml Back Pressure: 100 bar to keep the CO₂ in Supercritical flow) to obtain benzyl (3S,4R)-4-(2-tert-butoxy-2-oxo-ethyl)-3-methyl-piperidine-1-carboxylate (6B, 1.3 g, 3.74 mmol, 11% yield). After purified by SFC(Sample preparation: Add EtOH 25 ml into sample Instrument: Thar 80 SFC Mobile Phase: 25% EtOH (0.1% NH₃H₂O) in Supercritical CO₂ Flow Rate: 50 g/min Cycle Time: 4.9 min, total time: 25 min Single injection volume: 4.0 ml Back Pressure: 100 bar to keep the CO₂ in Supercritical flow) to obtain benzyl (3R,4R)-4-(2-tert-butoxy-2-oxo-ethyl)-3-methyl-piperidine-1-carboxylate (6C, 1 g, 2.88 mmol, 9% yield) and benzyl (3S,4S)-4-(2-tert-butoxy-2-oxo-ethyl)-3-methyl-piperidine-1-carboxylate (6A, 1 g, 2.88 mmol, 9% yield).

LCMS (ES+): m/z 348.1 [M+H]⁺

### Step 5: tert-butyl 2-[(3S,4S)-3-methyl-4-piperidyl]acetate (7A)

To a solution of benzyl (3S,4S)-4-(2-tert-butoxy-2-oxo-ethyl)-3-methyl-piperidine-1-carboxylate (6A, 500 mg, 1.44 mmol) in Methanol (10 mL) was added Pd/C (100 mg, 10% purity) under N₂ atmosphere. The suspension was degassed and purged with hydrogen three times. The mixture was stirred under hydrogen atmosphere (15 Psi) at 15° C. for 16 h. The reaction mixture was filtered and concentrated under reduced pressure to give tert-butyl 2-[(3S,4S)-3-methyl-4-piperidyl]acetate (7A, 300 mg, 1.41 mmol, 98% yield) as a white solid. The material was used in the next step without further purification. 1H NMR (400 MHz, CHLOROFORM-d) δ 3.09-2.89 (m, 2H), 2.65-2.61 (m, 1H), 2.50-2.45 (m, 1H), 2.31-2.25 (m, 1H), 2.01-1.93 (m, 1H), 1.76-1.69 (m, 1H), 1.63-1.58 (m, 1H), 1.47-1.45 (m, 9H), 1.34-1.16 (m, 2H), 0.87 (d, J=6.8 Hz, 3H)

### Step 6: tert-butyl 2-((3S,4S)-1-(1-(2,6-bis(benzyloxy)pyridin-3-yl)-3-methyl-2-oxo-2,3-dihydro-1H-benzo[d]imidazol-5-yl)-3-methylpiperidin-4-yl)acetate (9A)

To a solution of 1-(2,6-bis(benzyloxy)pyridin-3-yl)-5-bromo-3-methyl-1H-benzo[d]imidazol-2 (3H)-one (8, 100 mg, 193.65 μmol), tert-butyl 2-((3S,4S)-3-methylpiperidin-4-yl)acetate (7A, 50.00 mg, 234.39 μmol) in dioxane (2 mL) were added Cs₂CO₃ (157.74 mg, 484.13 μmol) and CPhos Pd G3 (15.62 mg, 19.37 μmol) under N₂. The mixture was stirred at 90° C. for 16 h under N₂. The reaction mixture was concentrated under vacuum. The residue was purified by column chromatography (SiO₂, petroleum ether/ethyl acetate=10/1 to 1/1; petroleum ether/ethyl acetate=1/1, R_{f}=0.4) to afford tert-butyl 2-((3S,4S)-1-(1-(2,6-bis(benzyloxy)pyridin-3-yl)-3-methyl-2-oxo-2,3-dihydro-1H-benzo[d]imidazol-5-yl)-3-methylpiperidin-4-yl)acetate (9A, 80 mg, 120.84 μmol, 62% yield) as a yellow solid.

LCMS (ESI): 471.2 m/z [M+H]+

¹H NMR (400 MHz, DMSO-d₆) δ 7.76 (d, J=8.4 Hz, 1H), 7.46-7.42 (m, 2H), 7.41-7.33 (m, 3H), 7.31-7.23 (m, 5H), 6.85 (d, J=2.0 Hz, 1H), 6.61-6.56 (m, 2H), 6.55-6.50 (m, 1H), 5.42-5.31 (m, 4H), 3.62-3.48 (m, 2H), 3.35 (s, 3H), 2.63-2.57 (m, 1H), 2.33-2.26 (m, 1H), 2.02-1.97 (m, 1H), 1.73 (br d, J=9.6 Hz, 1H), 1.53-1.34 (m, 13H), 0.91 (d, J=6.0 Hz, 3H).

### Step 7: tert-butyl 2-((3S,4S)-1-(1-(2,6-dioxopiperidin-3-yl)-3-methyl-2-oxo-2,3-dihydro-1H-benzo[d]imidazol-5-yl)-3-methylpiperidin-4-yl)acetate (10)

To a solution of tert-butyl 2-((3S,4S)-1-(1-(2,6-bis(benzyloxy)pyridin-3-yl)-3-methyl-2-oxo-2,3-dihydro-1H-benzo[d]imidazol-5-yl)-3-methylpiperidin-4-yl)acetate (9A, 30 mg, 46.24 μmol) in DMF (1 mL) were added Pd(OH)₂ (30 mg, 10% purity) and Pd/C (30 mg, 10% purity) under N₂ atmosphere. The suspension was degassed and purged with H₂ three times. The mixture was stirred under H₂ (15 psi) at 30° C. for 12 h. The reaction mixture was filtered and concentrated under vacuum. The residue was purified by prep-HPLC (flow: 25 mL/min; gradient: from 19-49% MeCN in water (0.1% TFA) over 12 min; column: Phenomenex Synergi C18 150×25 mm×10 um) to afford tert-butyl 2-((3S,4S)-1-(1-(2,6-dioxopiperidin-3-yl)-3-methyl-2-oxo-2,3-dihydro-1H-benzo[d]imidazol-5-yl)-3-methylpiperidin-4-yl)acetate (10, 8.42 mg, 14.26 μmol, 31% yield, TFA salt) as a white solid.

LCMS (ESI): 471.2 m/z [M+H]⁺

¹H NMR (400 MHz, DMSO-d₆) δ 11.10 (s, 1H), 7.78-6.48 (m, 3H), 5.47-5.26 (m, 1H), 3.64-3.41 (m, 3H), 3.34 (s, 3H), 2.95-2.84 (m, 1H), 2.77-2.60 (m, 2H), 2.57-2.51 (m, 2H), 2.11-1.97 (m, 2H), 1.93-1.80 (m, 1H), 1.79-1.50 (m, 3H), 1.42 (s, 9H), 0.93 (d, J=6.0 Hz, 3H).

### tert-butyl 2-[(3S,4R)-1-[1-(2,6-dioxo-3-piperidyl)-3-methyl-2-oxo-benzimidazol-5-yl]-3-methyl-4-piperidyl]acetate (4)

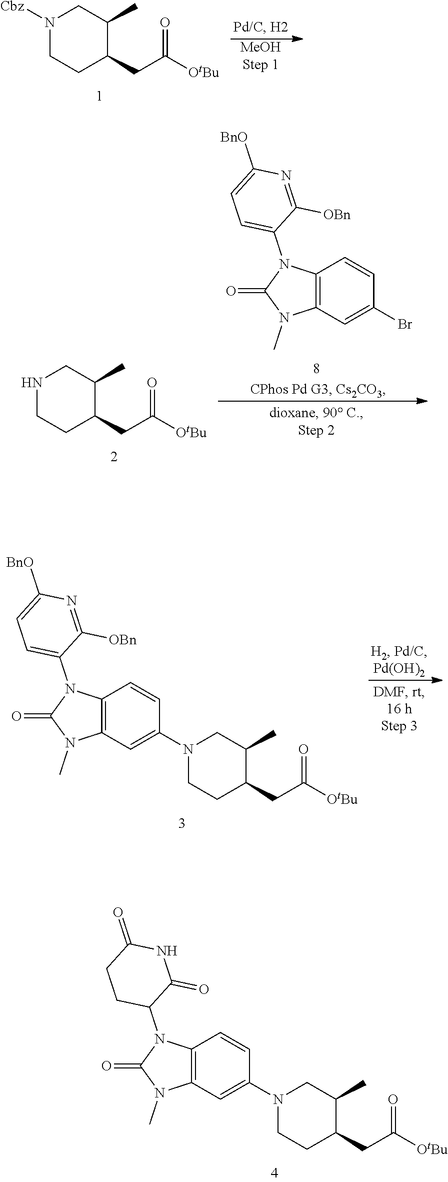 [SMILES: C[C@@H]1CN(C(=O)OCc2ccccc2)CC[C@@H]1CC(=O)OC(C)(C)C ; "MeOHStep 1" ; Cn1c(=O)n(-c2ccc(OCc3ccccc3)nc2OCc2ccccc2)c2ccc(Br)cc21 ; C[C@@H]1CNCC[C@@H]1CC(=O)OC(C)(C)C ; C[C@@H]1CN(c2ccc3c(c2)n(C)c(=O)n3-c2ccc(OCc3ccccc3)nc2OCc2ccccc2)CC[C@@H]1CC(=O)OC(C)(C)C ; C[C@@H]1CN(c2ccc3c(c2)n(C)c(=O)n3C2CCC(=O)NC2=O)CC[C@@H]1CC(=O)OC(C)(C)C]

tert-butyl 2-[(3 S,4R)-1-[1-(2,6-dioxo-3-piperidyl)-3-methyl-2-oxo-benzimidazol-5-yl]-3-methyl-4-piperidyl]acetate (4, 20.32 mg, 34.41 μmol, 56% yield, TFA salt) was prepared in three steps following the same procedure as tert-butyl 2-[(3S,4S)-1-[1-(2,6-dioxo-3-piperidyl)-3-methyl-2-oxo-benzimidazol-5-yl]-3-methyl-4-piperidyl]acetate.

LCMS (ES+): m/z 471.2 [M+H]⁺

1H NMR (400 MHz, DMSO-d6) δ=11.10 (s, 1H), 7.42-6.63 (m, 3H), 5.43-5.31 (m, 1H), 3.66-3.12 (m, 7H), 2.93-2.85 (m, 1H), 2.76-2.56 (m, 4H), 2.40-2.09 (m, 3H), 2.03-1.95 (m, 1H), 1.79-1.64 (m, 1H), 1.42 (s, 9H), 0.97 (br s, 3H).

### tert-butyl 2-[(3R,4R)-1-[1-(2,6-dioxo-3-piperidyl)-3-methyl-2-oxo-benzimidazol-5-yl]-3-methyl-4-piperidyl]acetate (4)

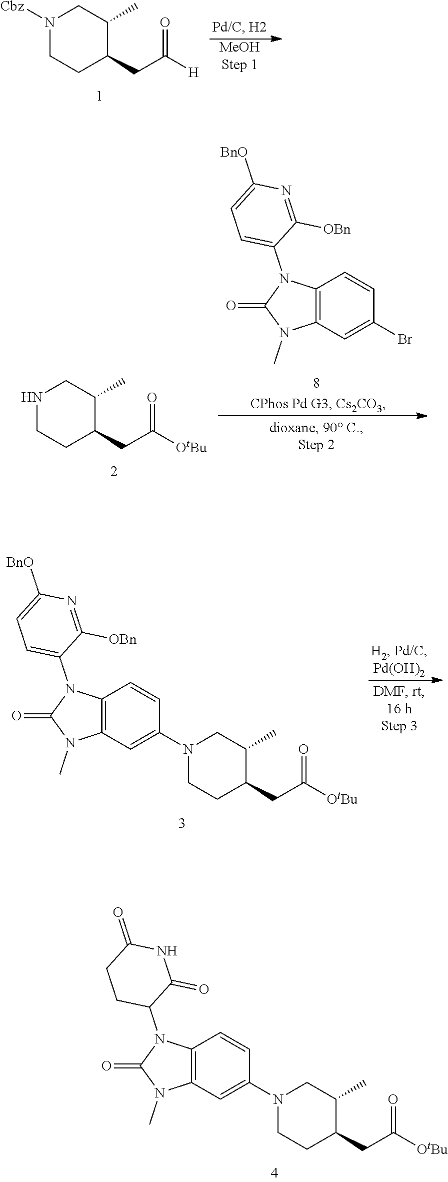 [SMILES: C[C@H]1CN(C(=O)OCc2ccccc2)CC[C@@H]1CC=O ; "MeOHStep 1" ; Cn1c(=O)n(-c2ccc(OCc3ccccc3)nc2OCc2ccccc2)c2ccc(Br)cc21 ; C[C@H]1CNCC[C@@H]1CC(=O)OC(C)(C)C ; C[C@H]1CN(c2ccc3c(c2)n(C)c(=O)n3-c2ccc(OCc3ccccc3)nc2OCc2ccccc2)CC[C@@H]1CC(=O)OC(C)(C)C ; C[C@H]1CN(c2ccc3c(c2)n(C)c(=O)n3C2CCC(=O)NC2=O)CC[C@@H]1CC(=O)OC(C)(C)C]

tert-butyl 2-[(3R,4R)-1-[1-(2,6-dioxo-3-piperidyl)-3-methyl-2-oxo-benzimidazol-5-yl]-3-methyl-4-piperidyl]acetate (4, 12.51 mg, 21.19 μmol, 46% yield, TFA salt) was prepared in three steps following the same procedure as tert-butyl 2-[(3S,4S)-1-[1-(2,6-dioxo-3-piperidyl)-3-methyl-2-oxo-benzimidazol-5-yl]-3-methyl-4-piperidyl]acetate.

LCMS (ES+): m/z 471.2 [M+H]⁺

1H NMR (400 MHz, DMSO-d6) δ 11.10 (s, 1H), 7.68-6.48 (m, 3H), 5.47-5.26 (m, 1H), 3.63-3.48 (m, 3H), 3.35 (s, 3H), 2.96-2.84 (m, 1H), 2.75-2.63 (m, 2H), 2.58-2.52 (m, 2H), 2.11-1.98 (m, 2H), 1.91-1.81 (m, 1H), 1.77-1.52 (m, 3H), 1.43 (s, 9H), 0.93 (d, J=6.0 Hz, 3H)

### tert-butyl 2-[(3R,4S)-1-[1-(2,6-dioxo-3-piperidyl)-3-methyl-2-oxo-benzimidazol-5-yl]-3-methyl-4-piperidyl]acetate (4)

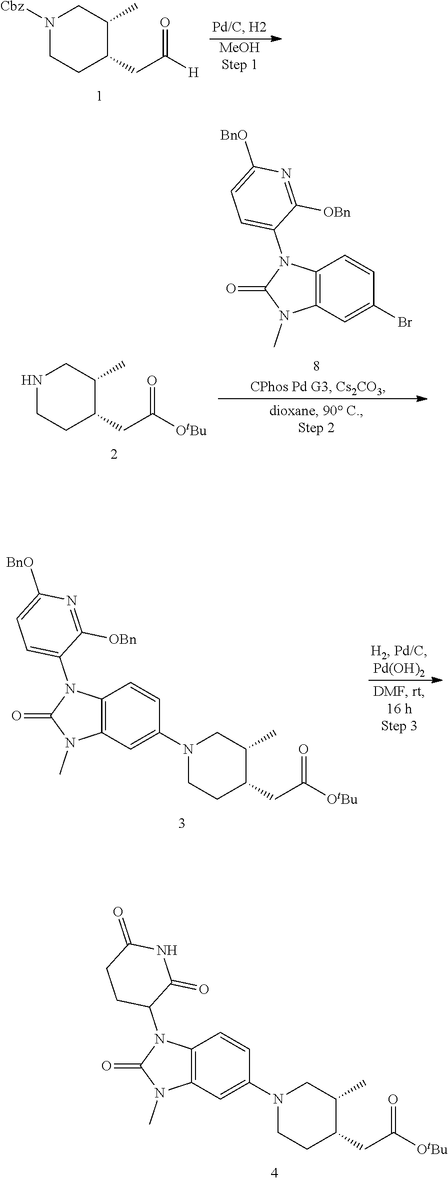 [SMILES: C[C@H]1CN(C(=O)OCc2ccccc2)CC[C@H]1CC=O ; "MeOHStep 1" ; Cn1c(=O)n(-c2ccc(OCc3ccccc3)nc2OCc2ccccc2)c2ccc(Br)cc21 ; C[C@H]1CNCC[C@H]1CC(=O)OC(C)(C)C ; C[C@H]1CN(c2ccc3c(c2)n(C)c(=O)n3-c2ccc(OCc3ccccc3)nc2OCc2ccccc2)CC[C@H]1CC(=O)OC(C)(C)C ; C[C@H]1CN(c2ccc3c(c2)n(C)c(=O)n3C2CCC(=O)NC2=O)CC[C@H]1CC(=O)OC(C)(C)C]

tert-butyl 2-[(3R,4S)-1-[1-(2,6-dioxo-3-piperidyl)-3-methyl-2-oxo-benzimidazol-5-yl]-3-methyl-4-piperidyl]acetate (4, 34.76 mg, 58.87 μmol, 48% yield, TFA salt) was prepared in three steps following the same procedure as tert-butyl 2-[(3S,4S)-1-[1-(2,6-dioxo-3-piperidyl)-3-methyl-2-oxo-benzimidazol-5-yl]-3-methyl-4-piperidyl]acetate.

LCMS (ES+): m/z 471.2 [M+H]⁺

1H NMR (400 MHz, DMSO-d6) δ=11.10 (s, 1H), 7.62-6.60 (m, 3H), 5.36 (br dd, J=4.4, 12.4 Hz, 1H), 3.57-2.98 (m, 7H), 2.95-2.85 (m, 1H), 2.75-2.60 (m, 2H), 2.44-2.25 (m, 2H), 2.16 (br s, 2H), 2.02-1.98 (m, 1H), 1.97-1.55 (m, 2H), 1.42 (s, 9H), 0.96 (br s, 3H)

### 2-[4-[1-(2,6-dioxo-3-piperidyl)-3-methyl-2-oxo-benzimidazol-5-yl]phenyl]acetic acid (3)

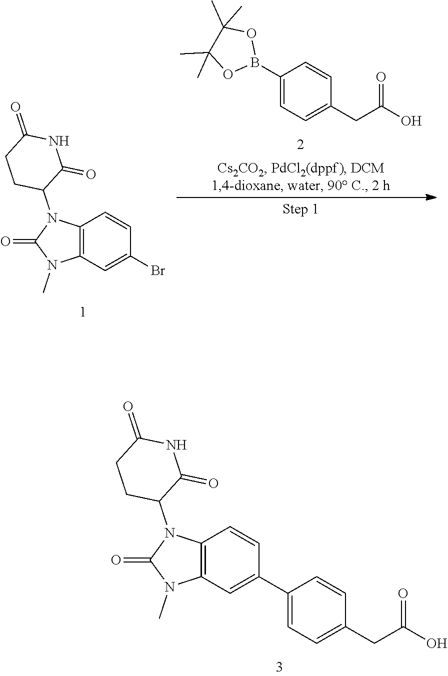 [SMILES: Cn1c(=O)n(C2CCC(=O)NC2=O)c2ccc(Br)cc21 ; CC1(C)OB(c2ccc(CC(=O)O)cc2)OC1(C)C ; Cn1c(=O)n(C2CCC(=O)NC2=O)c2ccc(-c3ccc(CC(=O)O)cc3)cc21]

### Step 1: 2-[4-[1-(2,6-dioxo-3-piperidyl)-3-methyl-2-oxo-benzimidazol-5-yl]phenyl]acetic acid (3)

Into a 25 mL pressure tube containing a well-stirred solution of 3-(5-bromo-3-methyl-2-oxo-benzimidazol-1-yl)piperidine-2,6-dione (1, 550 mg, 1.63 mmol) and 2-[4-(4,4,5,5-tetramethyl-1,3,2-dioxaborolan-2-yl)phenyl]acetic acid (2, 639.47 mg, 2.44 mmol) in 1,4-dioxane (7 mL) and water (0.75 mL) was added cesium carbonate (529.93 mg, 1.63 mmol). The mixture was degassed with nitrogen for 10 min. Pd(dppf)Cl₂·CH₂Cl₂ (199.23 mg, 243.97 μmol) was added. The tube was sealed and the mixture stirred at 90° C. for 16 h. The reaction mixture was filtered through Celite and washed with 1,4-dioxane (125 mL). The filtrate was concentrated under reduced pressure and the residue was purified by reverse phase column chromatography [Purification method: Siliasep C18 120 g, column; Mobile phase A: 0.1% formic acid in water; Mobile phase B: MeCN] to obtain 2-[4-[1-(2,6-dioxo-3-piperidyl)-3-methyl-2-oxo-benzimidazol-5-yl]phenyl]acetic acid (3, 250 mg, 603.72 μmol, 37% yield) as an off-white solid.

LCMS (ES+): m/z 394.0 [M+H]⁺.

### 2-[4-[1-(2,6-dioxo-3-piperidyl)-3-methyl-2-oxo-benzimidazol-5-yl]-3-methyl-phenyl]acetic acid (8)

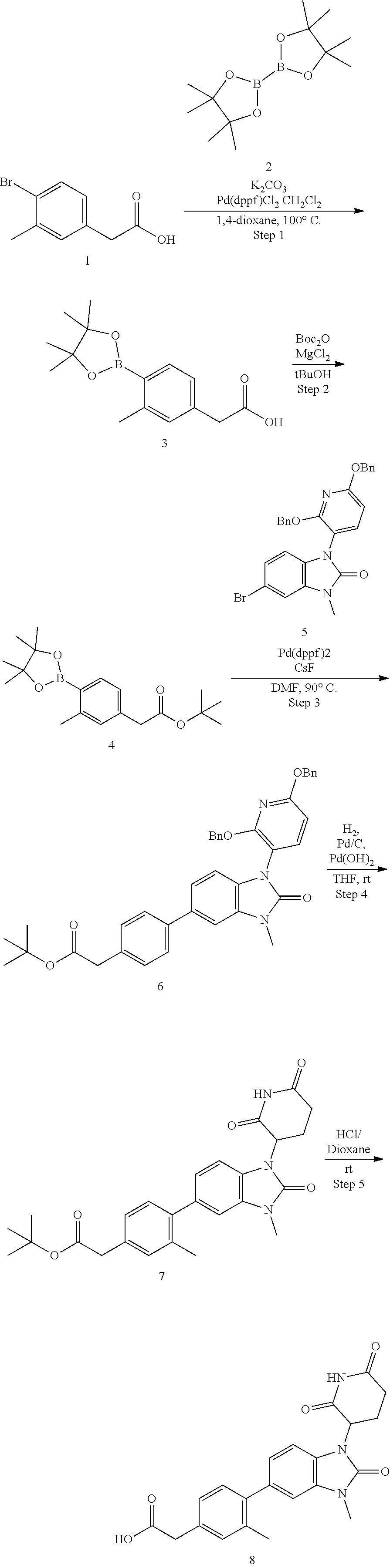

### Step 1: 2-[3-methyl-4-(4,4,5,5-tetramethyl-1,3,2-dioxaborolan-2-yl)phenyl]acetic acid (3)

To a solution of 2-(4-bromo-3-methyl-phenyl)acetic acid (1, 9 g, 39.29 mmol), 4,4,5,5-tetramethyl-2-(4,4,5,5-tetramethyl-1,3,2-dioxaborolan-2-yl)-1,3,2-dioxaborolane (2, 14.97 g, 58.93 mmol) in 1,4-dioxane (90 mL) was added potassium acetate (19.28 g, 196.45 mmol, 12.28 mL), cyclopentyl(diphenyl)phosphane; dichloropalladium; iron (1.44 g, 1.96 mmol), the mixture was stirred at 100° C. for 16 h under N2. The reaction mixture was poured into water (200 mL), extracted with Ethyl acetate (200 mL*2). The combined organic layers were washed with brine (100 mL*2), dried over Na₂SO₄, filtered and concentrated. The residue was purified by column chromatography (SiO2, Petroleum ether/Ethyl acetate=1/1) to afford 2-[3-methyl-4-(4,4,5,5-tetramethyl-1,3,2-dioxaborolan-2-yl)phenyl]acetic acid (3, 7 g, 25.35 mmol, 65% yield) as an off-white solid.

¹H NMR (400 MHz, DMSO-d₆) δ=12.31 (s, 1H), 7.56 (d, J=7.6 Hz, 1H), 7.09-7.01 (m, 2H), 3.52 (s, 2H), 2.44 (s, 3H), 1.29 (s, 12H)

### Step 2: tert-butyl 2-[3-methyl-4-(4,4,5,5-tetramethyl-1,3,2-dioxaborolan-2-yl)phenyl]acetate (4)

To a solution of 2-[3-methyl-4-(4,4,5,5-tetramethyl-1,3,2-dioxaborolan-2-yl)phenyl]acetic acid (3, 500 mg, 1.81 mmol) in tert-butyl alcohol (5 mL) was added tert-butoxycarbonyl tert-butyl carbonate (513.74 mg, 2.35 mmol, 540.21 μL), dichloromagnesium (17.24 mg, 181.07 μmol), the mixture was stirred at 20° C. for 16 h. The reaction mixture was poured into water (10 ml), extracted with Ethyl acetate (10 mL*2). The combined organic layers were washed with brine (10 mL*2), dried over Na₂SO₄, filtered and concentrated. The residue was purified by column chromatography (SiO2, Petroleum ether/Ethyl acetate=1/1) to afford tert-butyl 2-[3-methyl-4-(4,4,5,5-tetramethyl-1,3,2-dioxaborolan-2-yl)phenyl]acetate (4, 300 mg, 902.96 μmol, 50% yield) as a colorless oil.

¹H NMR (400 MHz, DMSO-d₆) δ=7.57 (d, J=8.0 Hz, 1H), 7.07-7.01 (m, 2H), 3.50 (s, 2H), 2.43 (s, 3H), 1.38 (s, 9H), 1.29 (s, 12H)

### Step 3: tert-butyl 2-[4-[1-(2,6-dibenzyloxy-3-pyridyl)-3-methyl-2-oxo-benzimidazol-5-yl]-3-methyl-phenyl]acetate (6)

To a solution of 5-bromo-1-(2,6-dibenzyloxy-3-pyridyl)-3-methyl-benzimidazol-2-one (5, 3 g, 5.81 mmol), tert-butyl 2-[3-methyl-4-(4,4,5,5-tetramethyl-1,3,2-dioxaborolan-2-yl)phenyl]acetate (4, 2.51 g, 7.55 mmol) in DMF (30 mL) was added CsF (1.15 g, 17.43 mmol) and cyclopentyl(diphenyl)phosphane; dichloropalladium; iron (212.55 mg, 290.48 μmol). The mixture was stirred at 90° C. for 16 h under N2. The reaction mixture was poured into water (200 mL), extracted with Ethyl acetate (200 mL*2). The combined organic layers were washed with brine (100 mL*2), dried over Na₂SO₄, filtered and concentrated. The residue was purified by column chromatography (SiO2, Petroleum ether/Ethyl acetate=5/1) to afford tert-butyl 2-[4-[1-(2,6-dibenzyloxy-3-pyridyl)-3-methyl-2-oxo-benzimidazol-5-yl]-3-methyl-phenyl]acetate (6, 3 g, 4.67 mmol, 80% yield) as a yellow oil.

LCMS (ES+): m/z 642.4 [M+H]⁺

### Step 4: tert-butyl 2-[4-[1-(2,6-dioxo-3-piperidyl)-3-methyl-2-oxo-benzimidazol-5-yl]-3-methyl-phenyl]acetate (7)

To a solution of tert-butyl 2-[4-[1-(2,6-dibenzyloxy-3-pyridyl)-3-methyl-2-oxo-benzimidazol-5-yl]-3-methyl-phenyl]acetate (6, 500 mg, 779.12 μmol) in THF (5 mL) was added Pd(OH)₂/C (50 mg, 10% purity), Pd/C (50 mg, 10% purity), the mixture was stirred at 20° C. for 16 h under H2 (15 psi). The mixture was filtered, concentrated and purified by reversed phase flash chromatography (flow: 35 ml/min; gradient: from 5%-60%, 20 min; 60%, 5 min [Water (0.1% TFA) in MeCN; column: I.D. 31 mm*H140 mm, Welch Ultimate XB_C18 20-40 μm; 120 Å) to afford tert-butyl 2-[4-[1-(2,6-dioxo-3-piperidyl)-3-methyl-2-oxo-benzimidazol-5-yl]-3-methyl-phenyl]acetate (7, 120 mg, 203.62 μmol, 26% yield, TFA salt) as a yellow solid.

LCMS (ES+): m/z 464.2 [M+H]⁺

### Step 5: 2-[4-[1-(2,6-dioxo-3-piperidyl)-3-methyl-2-oxo-benzimidazol-5-yl]-3-methyl-phenyl]acetic acid (8)

To a solution of tert-butyl 2-[4-[1-(2,6-dioxo-3-piperidyl)-3-methyl-2-oxo-benzimidazol-5-yl]-3-methyl-phenyl]acetate (7, 1.2 g, 2.59 mmol) in HCl\dioxane (12 mL), the mixture was stirred at 20° C. for 6 h. The mixture was concentrated and purified by reversed phase flash chromatography (flow: 85 ml/min; gradient: 0%-30%, 10 min; 30%, 18 min; [Water (0.1% TFA): Acetonitrile] in MeCN; column: I.D. 41 mm*H235 mm, Welch Ultimate XB_C18 20-40 μm; 120 Å) to afford 2-[4-[1-(2,6-dioxo-3-piperidyl)-3-methyl-2-oxo-benzimidazol-5-yl]-3-methyl-phenyl]acetic acid (8, 660 mg, 1.49 mmol, 57% yield, HCl salt) as a white solid.

LCMS (ES+): m/z 408.4 [M+H]⁺

### 2-[4-[1-(2,6-dioxo-3-piperidyl)-3-methyl-2-oxo-benzimidazol-5-yl]-2-oxo-piperazin-1-yl]acetic acid (8)

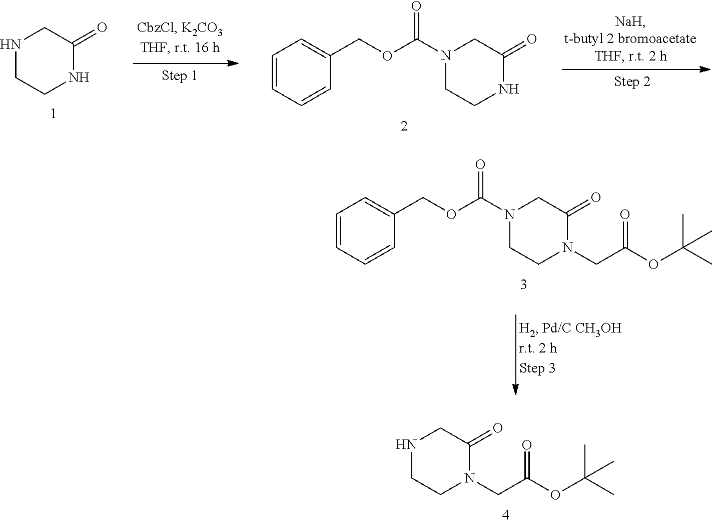 [SMILES: O=C1CNCCN1 ; O=C1CN(C(=O)OCc2ccccc2)CCN1 ; CC(C)(C)OC(=O)CN1CCN(C(=O)OCc2ccccc2)CC1=O ; CC(C)(C)OC(=O)CN1CCNCC1=O]

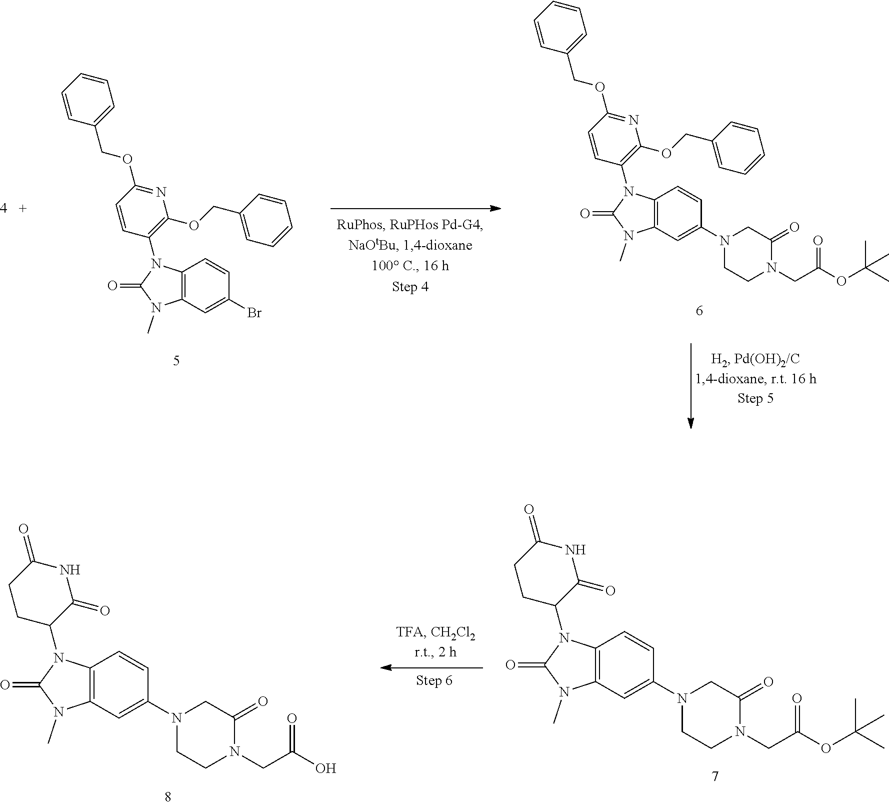 [SMILES: Cn1c(=O)n(-c2ccc(OCc3ccccc3)nc2OCc2ccccc2)c2ccc(Br)cc21 ; Cn1c(=O)n(-c2ccc(OCc3ccccc3)nc2OCc2ccccc2)c2ccc(N3CCN(CC(=O)OC(C)(C)C)C(=O)C3)cc21 ; Cn1c(=O)n(C2CCC(=O)NC2=O)c2ccc(N3CCN(CC(=O)O)C(=O)C3)cc21 ; Cn1c(=O)n(C2CCC(=O)NC2=O)c2ccc(N3CCN(CC(=O)OC(C)(C)C)C(=O)C3)cc21]

### Step 1: benzyl 3-oxopiperazine-1-carboxylate (2)

To a stirred solution of piperazin-2-one (1, 5 g, 49.94 mmol) in THF (50 mL) were added potassium carbonate (610.13 mg, 4.41 mmol) and benzyl carbonochloridate (50 wt. % solution in toluene) (18.74 g, 54.93 mmol). After 16 h, the reaction mixture was diluted with water (50 mL) and extracted with EtOAc (3×50 mL), washed with water (2×50 mL) and brine (25 mL). The organic layer was dried over Na₂SO₄ and evaporated. The residue was washed with MTBE to give benzyl 3-oxopiperazine-1-carboxylate (2, 10.2 g, 39.19 mmol, 78% yield) as a white solid.

### Step 2: benzyl 4-(2-tert-butoxy-2-oxo-ethyl)-3-oxo-piperazine-1-carboxylate (3)

To stirred solution of 60% sodium hydride (60% dispersion in oil) (1.24 g, 31.06 mmol) in THF (60 mL) was added a solution of benzyl 3-oxopiperazine-1-carboxylate (2, 5 g, 20.70 mmol) in THF (10 mL) at 0° C. under nitrogen atmosphere. After 5 min, tert-butyl 2-bromoacetate (4.85 g, 24.85 mmol, 3.64 mL) was added. After 2 h, the reaction was quenched with water (15 mL) and extracted with ethyl acetate (2×50 mL), washed with brine solution (2×50 mL), dried over Na₂SO₄ and then evaporated. The crude compound was purified with flash column chromatography (65% EtOAc in pet ether) to obtain benzyl 4-(2-tert-butoxy-2-oxo-ethyl)-3-oxo-piperazine-1-carboxylate (3, 5.3 g, 15.03 mmol, 73% yield) as a colorless solid.

LCMS (ES+): m/z 293.2 [M−tBu+H]⁺

### Step 3: tert-butyl 2-(2-oxopiperazin-1-yl)acetate (4)

To a stirred solution of benzyl 4-(2-tert-butoxy-2-oxo-ethyl)-3-oxo-piperazine-1-carboxylate (3, 5 g, 14.13 mmol) in methanol (10 mL) was added 10% palladium on carbon (1.5 g, 1.41 mmol). The suspension was stirred under hydrogen atmosphere for 16 h. The reaction mixture was filtered through Celite and washed it with methanol (100 mL), filtrate was collected and concentrated under reduced pressure to afford tert-butyl 2-(2-oxopiperazin-1-yl)acetate (4, 2.5 g, 11.08 mmol, 78% yield) as a colorless liquid.

### Step 4: tert-butyl 2-[4-[1-(2,6-dibenzyloxy-3-pyridyl)-3-methyl-2-oxo-benzimidazol-5-yl]-2-oxo-piperazin-1-yl]acetate (6)

To a stirred solution of 5-bromo-1-(2,6-dibenzyloxy-3-pyridyl)-3-methyl-benzimidazol-2-one (4, 800 mg, 1.55 mmol) in 1,4-dioxane (20 mL) was added tert-butyl 2-(2-oxopiperazin-1-yl)acetate (5, 1.05 g, 4.65 mmol). The reaction mixture was degassed with nitrogen for 5 min and sodium tert-butoxide (446.64 mg, 4.65 mmol), RuPhos (4.52 mg, 9.68 μmol), and RuPhos Pd-G4 (131.75 mg, 154.92 μmol) added. The reaction mixture was stirred at 110° C. for 16 h. The reaction mixture was filtered through Celite and washed with methanol (2×50 mL). The filtrate was concentrated under reduced pressure and purified via flash column chromatography (5% CH₃OH in CH₂Cl₂) to obtain tert-butyl 2-[4-[1-(2,6-dibenzyloxy-3-pyridyl)-3-methyl-2-oxo-benzimidazol-5-yl]-2-oxo-piperazin-1-yl]acetate (6, 530 mg, 739.61 μmol, 48% yield) as a brown liquid.

LCMS (ES+): m/z 650.2 [M+H]⁺

### Step 5: tert-butyl 2-[4-[1-(2,6-dioxo-3-piperidyl)-3-methyl-2-oxo-benzimidazol-5-yl]-2-oxo-piperazin-1-yl]acetate (7)

To a stirred solution of tert-butyl 2-[4-[1-(2,6-dibenzyloxy-3-pyridyl)-3-methyl-2-oxo-benzimidazol-5-yl]-2-oxo-piperazin-1-yl]acetate (6, 320 mg, 413.90 μmol) in 1,4-dioxane (15 mL) was added 20 wt. % palladium hydroxide on carbon (84.04 mg, 119.69 μmol) and the suspension was stirred under hydrogen atmosphere at room temperature for 16 h. The reaction mixture was filtered through Celite and washed it with 1,4-dioxane (100 mL). The filtrate was concentrated and purified by reverse phase preparatory HPLC (Column: XSelect C18 (250×19) mm, 5 micron; Mobile phase A: 0.1% Formic acid in water; Mobile phase B: MeCN] to afford tert-butyl 2-[4-[1-(2,6-dioxo-3-piperidyl)-3-methyl-2-oxo-benzimidazol-5-yl]-2-oxo-piperazin-1-yl]acetate (7, 210 mg, 384.35 μmol, 93% yield, Formic acid salt) as an off white solid.

LCMS (ES+): m/z 472.0 [M+H]⁺

### Step 6: 2-[4-[1-(2,6-dioxo-3-piperidyl)-3-methyl-2-oxo-benzimidazol-5-yl]-2-oxo-piperazin-1-yl]acetic acid (8)

To a stirred solution of crude tert-butyl 2-[4-[1-(2,6-dioxo-3-piperidyl)-3-methyl-2-oxo-benzimidazol-5-yl]-2-oxo-piperazin-1-yl]acetate (7, 170 mg, 212.72 μmol) in CH₂Cl₂ (2 mL) was added trifluoroacetic acid (2.22 g, 19.47 mmol, 1.50 mL) at 0° C. After 3 h, the volatiles were evaporated to dryness to obtain 2-[4-[1-(2,6-dioxo-3-piperidyl)-3-methyl-2-oxo-benzimidazol-5-yl]-2-oxo-piperazin-1-yl]acetic acid (8, 130 mg, 134.66 μmol, 63% yield, TFA salt) as a colorless solid.

LCMS (ES+): m/z 416.0 [M+H]⁺

### 2-[3-[1-(2,6-dioxo-3-piperidyl)-3-methyl-2-oxo-benzimidazol-5-yl]-4-fluoro-1-piperidyl]acetic acid (5)

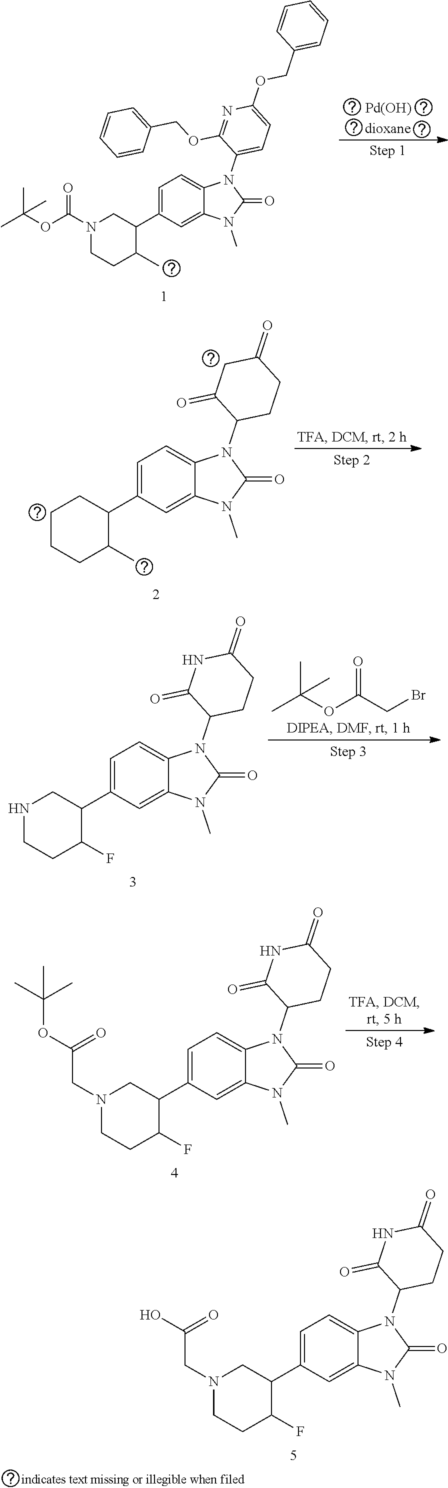

### Step 1: tert-butyl 3-[1-(2,6-dioxo-3-piperidyl)-3-methyl-2-oxo-benzimidazol-5-yl]-4-fluoro-piperidine-1-carboxylate (2)

Into a 100 mL single neck round bottom flask containing a well-stirred solution of tert-butyl 3-[1-(2,6-dibenzyloxy-3-pyridyl)-3-methyl-2-oxo-benzimidazol-5-yl]-4-fluoro-piperidine-1l-carboxylate (1, 700 mg, 1.10 mmol) in 1,4-dioxane (8 mL) was added Palladium hydroxide on carbon (20 wt. % 50% water) (461.74 mg, 657.56 μmol). The reaction mixture was stirred under hydrogen atmosphere at room temperature for 16 h. The reaction mixture was filtered through Celite, washed with 1,4-dioxane (100 mL) and ethyl acetate (150 mL). The filtrate was concentrated under reduced pressure to afford tert-butyl 3-[1-(2,6-dioxo-3-piperidyl)-3-methyl-2-oxo-benzimidazol-5-yl]-4-fluoro-piperidine-1-carboxylate (2, 450 mg, 967.43 Mol, 88% yield,) as an off-white solid.

LCMS (ES+): m/z 405.2 [M−tBu+H]⁺

### Step 2: 3-[5-(4-fluoro-3-piperidyl)-3-methyl-2-oxo-benzimidazol-1-yl]piperidine-2,6-dione (3)

Into a 100 mL single neck round bottom flask containing a well-stirred solution of tert-butyl 3-[1-(2,6-dioxo-3-piperidyl)-3-methyl-2-oxo-benzimidazol-5-yl]-4-fluoro-piperidine-1l-carboxylate (2, 450 mg, 967.43 μmol) in anhydrous DCM (3 mL) was added TFA (2.21 g, 19.35 mmol, 1.49 mL). After 2 h, the mixture was concentrated under vacuum and purified by reverse phase prep HPLC [Purification method: Column: XSelect C18 (150×19) mm, 5 micron; Mobile phase A: 0.1% TFA in water; Mobile phase B: MeCN] to afford 3-[5-(4-fluoro-3-piperidyl)-3-methyl-2-oxo-benzimidazol-1-yl]piperidine-2,6-dione (3,455 mg, 957.47 μmol, 99% yield, TFA salt) as an off-white solid.

LCMS (ES+): m/z 361.2 [M+H]⁺

### Step 3: tert-butyl 2-[3-[1-(2,6-dioxo-3-piperidyl)-3-methyl-2-oxo-benzimidazol-5-yl]-4-fluoro-1-piperidyl]acetate (4)

Into a 20 mL vial containing a well-stirred solution of 3-[5-(4-fluoro-3-piperidyl)-3-methyl-2-oxo-benzimidazol-1-yl]piperidine-2,6-dione (3, 200 mg, 408.93 μmol, TFA salt) in DMF (2 mL) were added DIPEA (158.55 mg, 1.23 mmol, 213.68 μL) and tert-butyl 2-bromoacetate (63.81 mg, 327.15 μmol, 47.98 μL) at 0° C. The reaction mixture was stirred at ambient temperature for 1 h. The reaction mixture was concentrated under vacuum and diluted with water. The solid was filtered and washed with water and MTBE and dried under vacuum to afford tert-butyl 2-[3-[1-(2,6-dioxo-3-piperidyl)-3-methyl-2-oxo-benzimidazol-5-yl]-4-fluoro-1-piperidyl]acetate (4, 110 mg, 223.03 μmol, 55% yield) as an off-white solid.

LCMS (ES+): m/z 475.2 [M+H]⁺

### Step 4: 2-[3-[1-(2,6-dioxo-3-piperidyl)-3-methyl-2-oxo-benzimidazol-5-yl]-4-fluoro-1-piperidyl]acetic acid (5)

Into a 50 mL single neck round bottom flask containing well-stirred solution of tert-butyl 2-[3-[1-(2,6-dioxo-3-piperidyl)-3-methyl-2-oxo-benzimidazol-5-yl]-4-fluoro-1-piperidyl]acetate (4, 160 mg, 323.69 μmol) in anhydrous DCM (2 mL) was added TFA (738.15 mg, 6.47 mmol, 498.75 μL). After 5 h the volatiles were removed to obtain 2-[3-[1-(2,6-dioxo-3-piperidyl)-3-methyl-2-oxo-benzimidazol-5-yl]-4-fluoro-1-piperidyl]acetic acid (5, 170 mg, 300.70 μmol, 93% yield, TFA Salt) as an off white solid. The material was used in the next step without further purification.

LCMS (ES+): m/z 419.2 [M+H]⁺

### tert-butyl (3R,4R)-3-[1-(2,6-dibenzyloxy-3-pyridyl)-3-methyl-2-oxo-benzimidazol-5-yl]-4-fluoro-piperidine-1-carboxylate (2) and tert-butyl (3S,4S)-3-[1-(2,6-dibenzyloxy-3-pyridyl)-3-methyl-2-oxo-benzimidazol-5-yl]-4-fluoro-piperidine-1-carboxylate (3)

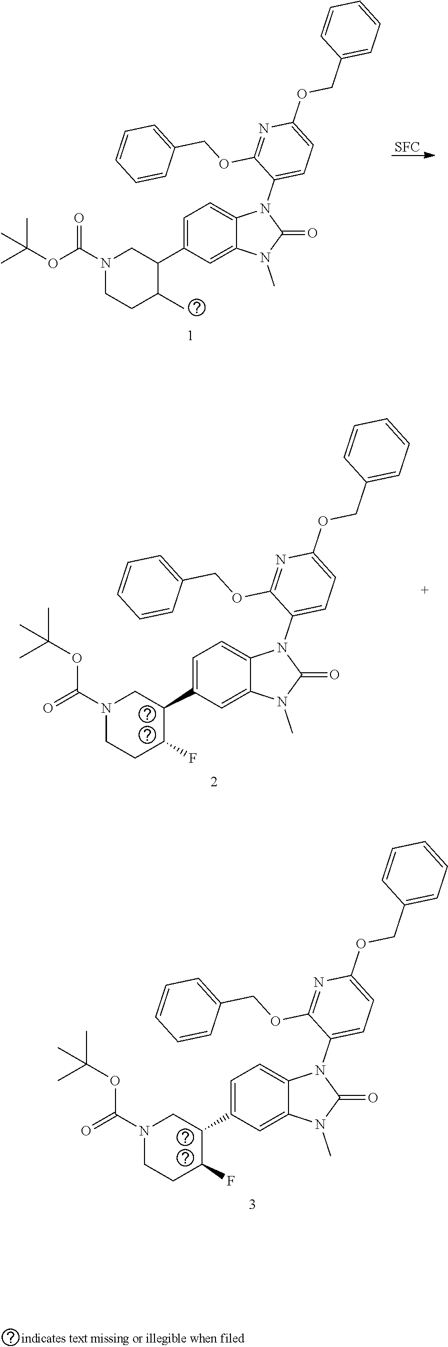 [SMILES: CC1CCN(C(=O)OC(C)(C)C)CC1c1ccc2c(c1)n(C)c(=O)n2-c1ccc(OCc2ccccc2)nc1OCc1ccccc1 ; "SFC" ; Cn1c(=O)n(-c2ccc(OCc3ccccc3)nc2OCc2ccccc2)c2ccc([C@@H]3CN(C(=O)OC(C)(C)C)CC[C@H]3F)cc21 ; Cn1c(=O)n(-c2ccc(OCc3ccccc3)nc2OCc2ccccc2)c2ccc([C@H]3CN(C(=O)OC(C)(C)C)CC[C@@H]3F)cc21]

tert-butyl 3-[1-(2,6-dibenzyloxy-3-pyridyl)-3-methyl-2-oxo-benzimidazol-5-yl]-4-fluoro-piperidine-1-carboxylate (1, 1.4 g) was dissolved in 0.5% Isopropyl Amine in Methanol and subjected to chiral SFC. Column Name: Lux A1, Flowrate: 4 mL/min, Co-Solvent: 30%, Co-Solvent Name: 0.5% Isopropyl Amine in Methanol, Outlet Pressure: 100 bar, Injected Volume: 2 μl, Temperature: 35° C.

Diastereomer 1: tert-butyl (3R,4R)-3-[1-(2,6-dibenzyloxy-3-pyridyl)-3-methyl-2-oxo-benzimidazol-5-yl]-4-fluoro-piperidine-1-carboxylate (2, 575 mg, 891.23 μmol, fast eluting fraction) as an off white solid.

Diastereomer 2: tert-butyl (3S,4S)-3-[1-(2,6-dibenzyloxy-3-pyridyl)-3-methyl-2-oxo-benzimidazol-5-yl]-4-fluoro-piperidine-1-carboxylate (3, 500 mg, late eluting fraction) as an off white solid.

### 2-[4-[1-(2,6-dioxo-3-piperidyl)-3-methyl-2-oxo-benzimidazol-5-yl]oxyphenyl]acetic acid (5)

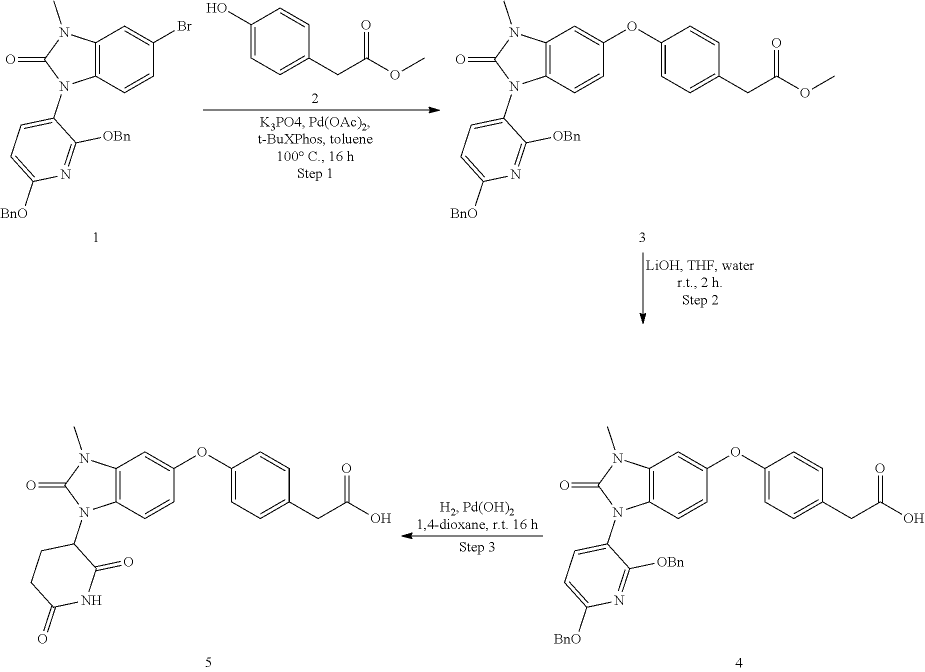

### Step 1: methyl 2-[4-[1-(2,6-dibenzyloxy-3-pyridyl)-3-methyl-2-oxo-benzimidazol-5-yl]oxyphenyl]acetate (3)

Into a 50 mL sealed tube containing a well-stirred solution of 5-bromo-1-(2,6-dibenzyloxy-3-pyridyl)-3-methyl-benzimidazol-2-one (1, 500 mg, 968.27 μmol) and methyl 2-(4-hydroxyphenyl)acetate (2, 160.90 mg, 968.27 μmol) in anhydrous toluene (15 mL) was added potassium phosphate tribasic anhydrous (822.12 mg, 3.87 mmol) at room temperature. The mixture was degassed by purging with nitrogen gas for 15 min. Then Palladium (II) acetate (65.22 mg, 290.48 μmol) and tBuXPhos (123.35 mg, 290.48 μmol) were added and the reaction mixture was stirred at 100° C. for 16 h. Reaction mixture was filtered through Celite and the filtrate was concentrated under reduced pressure. The crude compound was purified by flash silica gel column chromatography (30% EtOAc in pet ether) to afford methyl 2-[4-[1-(2,6-dibenzyloxy-3-pyridyl)-3-methyl-2-oxo-benzimidazol-5-yl]oxyphenyl]acetate (3, 220 mg, 357.69 μmol, 37% yield) as light brown sticky solid.

LCMS (ES+): m/z 602.2 [M+H]⁺

### Step 2: 2-[4-[1-(2,6-dibenzyloxy-3-pyridyl)-3-methyl-2-oxo-benzimidazol-5-yl]oxyphenyl]acetic acid (4)

Into a 25 mL single neck round bottom flask containing a well-stirred solution of methyl 2-[4-[1-(2,6-dibenzyloxy-3-pyridyl)-3-methyl-2-oxo-benzimidazol-5-yl]oxyphenyl]acetate (3, 220 mg, 354.69 μmol) in THF (2 mL) and water (1.5 mL) was added lithium hydroxide monohydrate (29.77 mg, 709.39 μmol). After 2 h, the mixture was concentrated under reduced pressure and acidified with 1.5 N HCl. The aqueous layer was extracted with EtOAC (2×25 mL). Combined organic layer was dried over anhydrous Na₂SO₄, filtered and concentrated under reduced pressure to afford 2-[4-[1-(2,6-dibenzyloxy-3-pyridyl)-3-methyl-2-oxo-benzimidazol-5-yl]oxyphenyl]acetic acid (4, 194 mg, 314.99 μmol, 89% yield) as pale yellow solid.

LCMS (ES+): m/z 588.0 [M+H]⁺

### Step 3: 2-[4-[1-(2,6-dioxo-3-piperidyl)-3-methyl-2-oxo-benzimidazol-5-yl]oxyphenyl]acetic acid (5)

Into a 50 mL single neck round bottom flask containing a well-stirred solution of 2-[4-[1-(2,6-dibenzyloxy-3-pyridyl)-3-methyl-2-oxo-benzimidazol-5-yl]oxyphenyl]acetic acid (4, 195 mg, 316.62 μmol) in anhydrous 1,4-dioxane (5 mL) was added 20 wt. % palladium hydroxide on carbon (200 mg, 284.83 μmol). The suspension was stirred at room temperature under hydrogen atmosphere for 16 h. The reaction mixture was filtered through Celite and concentrated under reduced pressure. The crude compound was purified by reverse phase column chromatography [Silicycle C18 column; Mobile phase A: 0.1% Formic acid in water; Mobile phase B: MeCN] to afford 2-[4-[1-(2,6-dioxo-3-piperidyl)-3-methyl-2-oxo-benzimidazol-5-yl]oxyphenyl]acetic acid (5, 98 mg, 237.80 μmol, 75% yield) as white solid.

LCMS (ES+): m/z 410.2 [M+H]⁺.

### 3-[5-[4-(2-aminoethyl)-1-piperidyl]-4-fluoro-3-methyl-2-oxo-benzimidazol-1-yl]piperidine-2,6-dione (5)

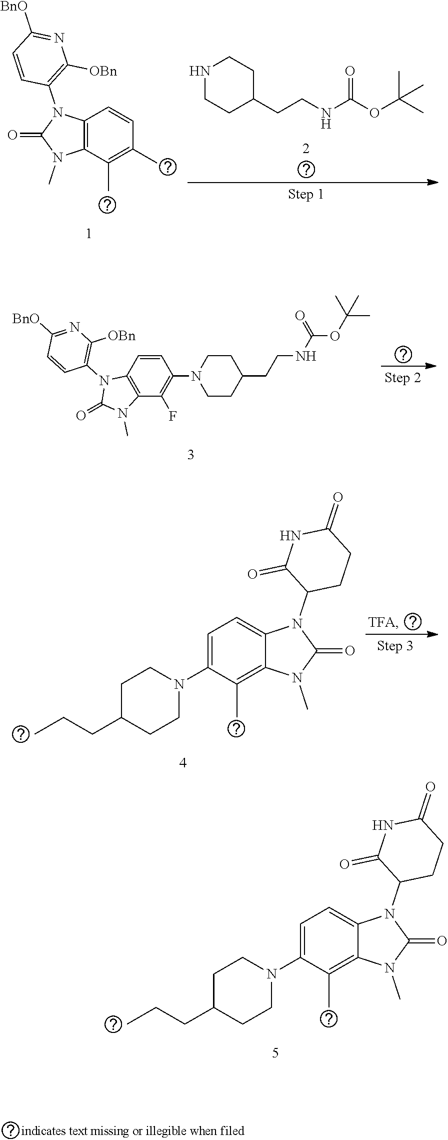

### Step 1: tert-butyl N-[2-[1-[1-(2,6-dibenzyloxy-3-pyridyl)-4-fluoro-3-methyl-2-oxo-benzimidazol-5-yl]-4-piperidyl]ethyl]carbamate (3)

Into a 25 mL pressure tube containing a well-stirred solution of 5-bromo-1-(2,6-dibenzyloxy-3-pyridyl)-4-fluoro-3-methyl-benzimidazol-2-one (1, 600.00 mg, 1.06 mmol) and tert-butyl N-[2-(4-piperidyl)ethyl]carbamate (2, 481.98 mg, 2.11 mmol) in anhydrous 1,4-dioxane (8 mL) was added cesium carbonate (1.03 g, 3.17 mmol). Nitrogen gas was bubbled through the reaction mixture for 5 min. Then CPhos Pd G3 (170.20 mg, 211.09 Mol) was added and again degassed for 5 min. The reaction mixture was heated to 120° C. for 16 h. The reaction mixture was filtered through Celite and washed with EtOAc (500 mL). The filtrate was concentrated under reduced pressure and purified by flash silica gel (230-400 mesh) column chromatography (50% EtOAc in pet ether) to afford tert-butyl N-[2-[1-[1-(2,6-dibenzyloxy-3-pyridyl)-4-fluoro-3-methyl-2-oxo-benzimidazol-5-yl]-4-piperidyl]ethyl]carbamate (3, 300 mg, 419.56 Mol, 40% yield) as an off white solid.

LCMS (ES+): m/z 682.2 [M+H]⁺

### Step 2: tert-butyl N-[2-[1-[1-(2,6-dioxo-3-piperidyl)-4-fluoro-3-methyl-2-oxo-benzimidazol-5-yl]-4-piperidyl]ethyl]carbamate (4)

Into a 50 mL single neck round bottom flask containing well-stirred solution of tert-butyl N-[2-[1-[1-(2,6-dibenzyloxy-3-pyridyl)-4-fluoro-3-methyl-2-oxo-benzimidazol-5-yl]-4-piperidyl]ethyl]carbamate (3, 350 mg, 487.68 μmol) in anhydrous 1,4-dioxane (5 mL) was added 20 wt. % palladium hydroxide on carbon (342.45 mg, 487.68 μmol). The resulting suspension was stirred at room temperature under hydrogen atmosphere for 16 h. The mixture was filtered through Celite and washed with 1,4-dioxane (100 mL). The filtrate was concentrated under reduced pressure and washed with diethyl ether (5 mL) to afford tert-butyl N-[2-[1-[1-(2,6-dioxo-3-piperidyl)-4-fluoro-3-methyl-2-oxo-benzimidazol-5-yl]-4-piperidyl]ethyl]carbamate (4, 240 mg, 457.11 μmol, 94% yield) as an off white solid.

LCMS (ES+): m/z 504.2 [M+H]⁺

### Step 3: 3-[5-[4-(2-aminoethyl)-1-piperidyl]-4-fluoro-3-methyl-2-oxo-benzimidazol-1-yl]piperidine-2,6-dione (5)

Into a 50 mL single neck round bottom flask containing a well-stirred solution of tert-butyl N-[2-[1-[1-(2,6-dioxo-3-piperidyl)-4-fluoro-3-methyl-2-oxo-benzimidazol-5-yl]-4-piperidyl]ethyl]carbamate (4, 242.11 mg, 456.74 μmol) in anhydrous DCM (3 mL) was added TFA (520.78 mg, 4.57 mmol, 351.88 μL). After 2 h, the reaction mixture was concentrated under reduced pressure and purified by reverse phase column chromatography [Siliasep C18 120 g; Mobile phase A: 0.1% formic acid in water; Mobile phase B: MeCN] to afford 3-[5-[4-(2-aminoethyl)-1-piperidyl]-4-fluoro-3-methyl-2-oxo-benzimidazol-1-yl]piperidine-2,6-dione (5, 150 mg, 329.92 μmol, 72% yield, formic acid salt) as an off-white solid.

LCMS (ES+): m/z 404.1 [M+H]⁺

### 4-[3-(2,6-dioxo-3-piperidyl)-1-methyl-2-oxo-8,9-dihydro-7H-imidazo[4,5-f]quinolin-6-yl]butanoic acid (4)

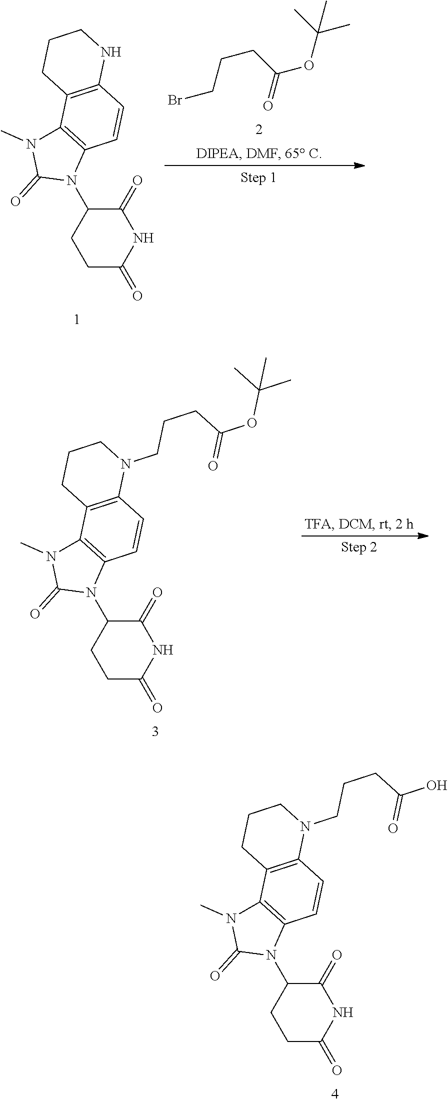

### Step 1: tert-butyl 4-[3-(2,6-dioxo-3-piperidyl)-1-methyl-2-oxo-8,9-dihydro-7H-imidazo[4,5-f]quinolin-6-yl]butanoate (3)

Into a 8 mL vial containing a well-stirred solution of 3-(1-methyl-2-oxo-6,7,8,9-tetrahydroimidazo[4,5-f]quinolin-3-yl)piperidine-2,6-dione (1, 100 mg, 292.68 μmol) in DMF (2 mL) was added DIPEA (113.48 mg, 878.03 μmol, 152.94 μL) and tert-butyl 4-bromobutanoate (2, 130.60 mg, 585.36 μmol, 103.65 μL) at 0° C. The reaction mixture was heated at 65° C. for 24 h. The solvent was removed under vacuum and compound was purified by reverse phase column chromatography [Silicycle C18 column; Mobile phase A: 0.1% formic acid in water; Mobile B: MeCN] to afford tert-butyl 4-[3-(2,6-dioxo-3-piperidyl)-1-methyl-2-oxo-8,9-dihydro-7H-imidazo[4,5-f]quinolin-6-yl]butanoate (3, 110 mg, 218.73 μmol, 75% yield, Formic acid salt) as a pink color solid.

LCMS (ES+): m/z 457.2 [M+H]⁺

### Step 2: 4-[3-(2,6-dioxo-3-piperidyl)-1-methyl-2-oxo-8,9-dihydro-7H-imidazo[4,5-f]quinolin-6-yl]butanoic acid (4)

Into a 50 mL single neck round bottom flask containing a well-stirred solution of tert-butyl 4-[3-(2,6-dioxo-3-piperidyl)-1-methyl-2-oxo-8,9-dihydro-7H-imidazo[4,5-f]quinolin-6-yl]butanoate (3, 140 mg, 275.79 μmol) in DCM (2.5 mL) was added TFA (628.93 mg, 5.52 mmol, 424.95 μL). After 2 h, the volatiles were removed and the residue washed with MTBE (50 mL). The material was purified by reverse phase prep HPLC [Purification method: Column: X-Select C18 (150×19) mm, 5 micron; Mobile phase A: 0.1% Formic Acid in water; Mobile phase B: MeCN] to afford 4-[3-(2,6-dioxo-3-piperidyl)-1-methyl-2-oxo-8,9-dihydro-7H-imidazo[4,5-f]quinolin-6-yl]butanoic acid (4, 120 mg, 267.87 μmol, 97% yield, Formic acid salt) as an off white solid.

UP-LCMS (ES+): m/z 401.2 [M+H]⁺

### 5-[3-(2,6-dioxo-3-piperidyl)-1-methyl-2-oxo-8,9-dihydro-7H-imidazo[4,5-f]quinolin-6-yl]pentanoic acid (4)

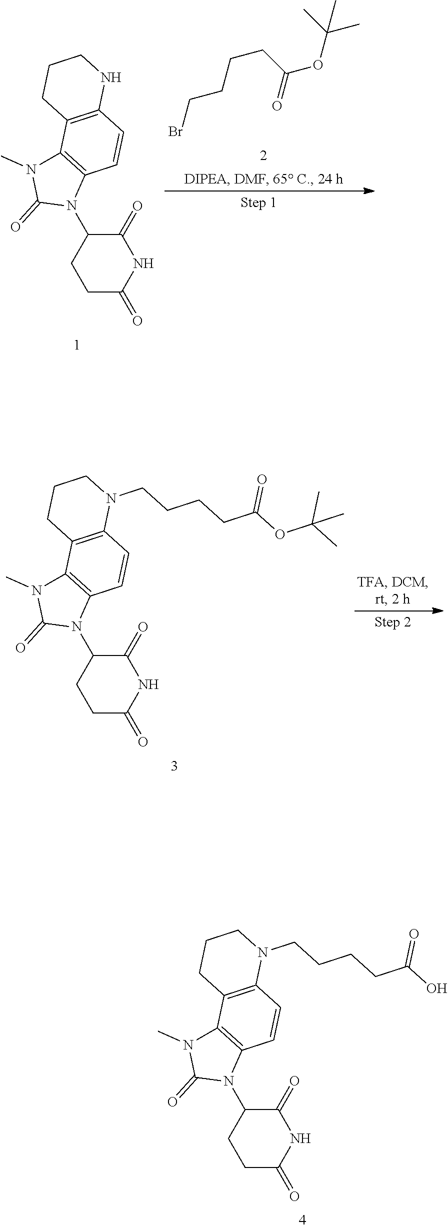

5-[3-(2,6-dioxo-3-piperidyl)-1-methyl-2-oxo-8,9-dihydro-7H-imidazo[4,5-f]quinolin-6-yl]pentanoic acid (4, 79 mg, 171.08 Mol, formic acid salt) was synthesized following the same two step procedure as 4-[3-(2,6-dioxo-3-piperidyl)-1-methyl-2-oxo-8,9-dihydro-7H-imidazo[4,5-f]quinolin-6-yl]butanoic acid.

LCMS (ES+): m/z 415.2 [M+H]⁺

### 2-[1-[1-(2,6-dioxo-3-piperidyl)-3-methyl-2-oxo-imidazo[4,5-b]pyridin-5-yl]-4-piperidyl]acetic acid (8)

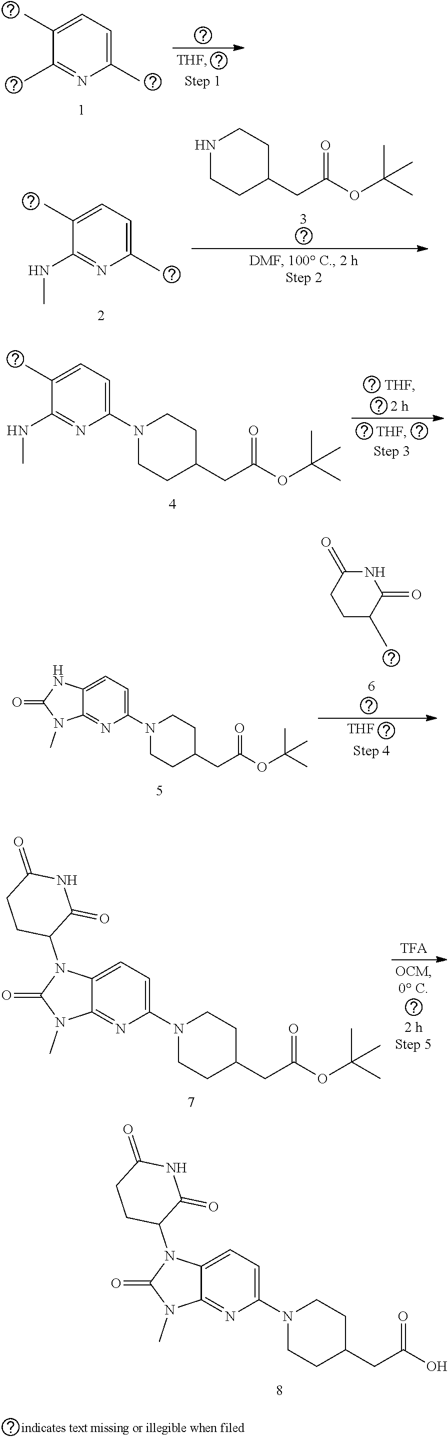

### Step 1: 6-bromo-N-methyl-3-nitro-pyridin-2-amine (2)

In to a 250 mL round bottom flask containing a well-stirred solution of 2,6-dibromo-3-nitro-pyridine (1, 10 g, 35.47 mmol) in anhydrous THF at 0° C. was added sodium carbonate (9.40 g, 88.69 mmol) and methylamine solution (2.0 M in THF, 42.57 mmol, 21.3 mL). The resulting mixture was stirred at rt for 30 min. The reaction mixture was filtered through Celite and washed with EtOAc (100×2 mL). The filtrate was concentrated under reduced pressure and purified by silica gel column chromatography (15-20% EtOAc in petroleum ether) to afford 6-bromo-N-methyl-3-nitro-pyridin-2-amine (2, 6.5 g, 25.77 mmol, 73% yield) as a yellow solid.

LCMS (ES+): m/z 351.5 [M+H]⁺

### Step 2: tert-butyl 2-[1-[6-(methylamino)-5-nitro-2-pyridyl]-4-piperidyl]acetate (4)

Into a 100 mL single neck round bottom flask containing well-stirred solution of 6-bromo-N-methyl-3-nitro-pyridin-2-amine (2, 4 g, 15.86 mmol) and tert-butyl 2-(4-piperidyl)acetate (3, 4.74 g, 23.79 mmol) in dry DMF (50 mL) was added cesium carbonate (15.50 g, 47.58 mmol) under nitrogen atmosphere at ambient temperature. The reaction was stirred at 100° C. for 2 h. The reaction mixture was diluted with ice-cold water (50 mL) and extracted with EtOAc (2×100 mL). The organic layers were separated, washed with brine (100 mL), dried over sodium sulfate and concentrated under reduced pressure. The material was purified by silica gel flash column chromatography (20% EtOAc in pet ether) to afford tert-butyl 2-[1-[6-(methylamino)-5-nitro-2-pyridyl]-4-piperidyl]acetate (4, 5 g, 14.13 mmol, 89% yield) as a yellow solid.

LCMS (ES+): m/z 351.5 [M+H]⁺

### Step 3: tert-butyl 2-[4-(3-methyl-2-oxo-1H-imidazo[4,5-b]pyridin-5-yl)-1-piperidyl]acetate (5)

Into a 50 mL single neck round bottom flask containing a well-stirred solution of tert-butyl 2-[1-[6-(methylamino)-5-nitro-2-pyridyl]-4-piperidyl]acetate (4, 808.08 mg, 2.28 mmol) in THF (15 mL) was added palladium on carbon (5% wt dry basis) (728.88 mg, 6.85 mmol) under nitrogen atmosphere at ambient temperature. The resultant mixture was stirred at ambient temperature for 2 h under hydrogen atmosphere. The reaction mixture was filtered using a syringe filter and washed with THF (25 mL). To this filtrate di(imidazol-1-yl)methanone (910.87 mg, 5.62 mmol) was added and stirred at ambient temperature for 16 h. The reaction mixture was concentrated to dryness and purified by silica gel column chromatography (60% EtOAc in petroleum ether) to yield tert-butyl 2-[4-(3-methyl-2-oxo-1H-imidazo[4,5-b]pyridin-5-yl)-1-piperidyl]acetate (5, 540 mg, 1.50 mmol, 67% yield).

LCMS (ES+): m/z 347.5 [M+H]⁺

### Step 4: tert-butyl 2-[1-[1-(2,6-dioxo-3-piperidyl)-3-methyl-2-oxo-imidazo[4,5-b]pyridin-5-yl]-4-piperidyl]acetate (7)

Into a 250 mL two neck round bottom flask containing a well-stirred suspension of tert-butyl 2-[1-(3-methyl-2-oxo-1H-imidazo[4,5-b]pyridin-5-yl)-4-piperidyl]acetate (5, 604.17 mg, 1.67 mmol) in anhydrous THF (35 mL) at 0° C. was added sodium hydride (60% suspension in oil) (669.64 mg, 16.74 mmol) and the resulting mixture was stirred for 1 h at room temperature. Afterwards, 3-bromopiperidine-2,6-dione (6, 1.43 g, 6.70 mmol) in THF (5 mL) was added at 0° C. and the mixture was heated at 65° C. for 16 h. The reaction was quenched with saturated ammonium chloride and extracted with EtOAc (3×60 mL). Drying over sodium sulfate and concentration under reduced pressure afforded crude compound that was purified by reverse phase preparative HPLC [Purification method: Column: X Bridge C 8 (150×19.1 mm) 5 um; Mobile phase: 0.1% TFA in water; Mobile phase B: Acetonitrile] to afford tert-butyl 2-[1-[1-(2,6-dioxo-3-piperidyl)-3-methyl-2-oxo-imidazo[4,5-b]pyridin-5-yl]-4-piperidyl]acetate (7, 490 mg, 856.81 μmol, 51% yield, TFA salt) as a white solid.

LCMS (ES+): m/z 458.2 [M+H]⁺

### Step 5: 2-[1-[1-(2,6-dioxo-3-piperidyl)-3-methyl-2-oxo-imidazo[4,5-b]pyridin-5-yl]-4-piperidyl]acetic acid (8)

Into a 50 mL single neck round bottom flask containing a well-stirred solution of tert-butyl 2-[1-[1-(2,6-dioxo-3-piperidyl)-3-methyl-2-oxo-imidazo[4,5-b]pyridin-5-yl]-4-piperidyl]acetate (7, 230 mg, 497.68 μmol) in anhydrous DCM (2 mL) at 0° C. was added trifluoroacetic acid (567.47 mg, 4.98 mmol, 383.43 μL) dropwise. The reaction mixture was stirred at room temperature for 2 h. The reaction mixture was concentrated under reduced pressure and triturated with MTBE to obtain 2-[1-[1-(2,6-dioxo-3-piperidyl)-3-methyl-2-oxo-imidazo[4,5-b]pyridin-5-yl]-4-piperidyl]acetic acid (8, 220 mg, 390.33 μmol, 78% yield, TFA salt) as an off-white solid.

LCMS (ES+): m/z 402.6 [M+H]⁺

### 2-[(3S)-4-[1-(2,6-dioxo-3-piperidyl)-4-fluoro-3-methyl-2-oxo-benzimidazol-5-yl]-3-methyl-piperazin-1-yl]acetic acid (12)

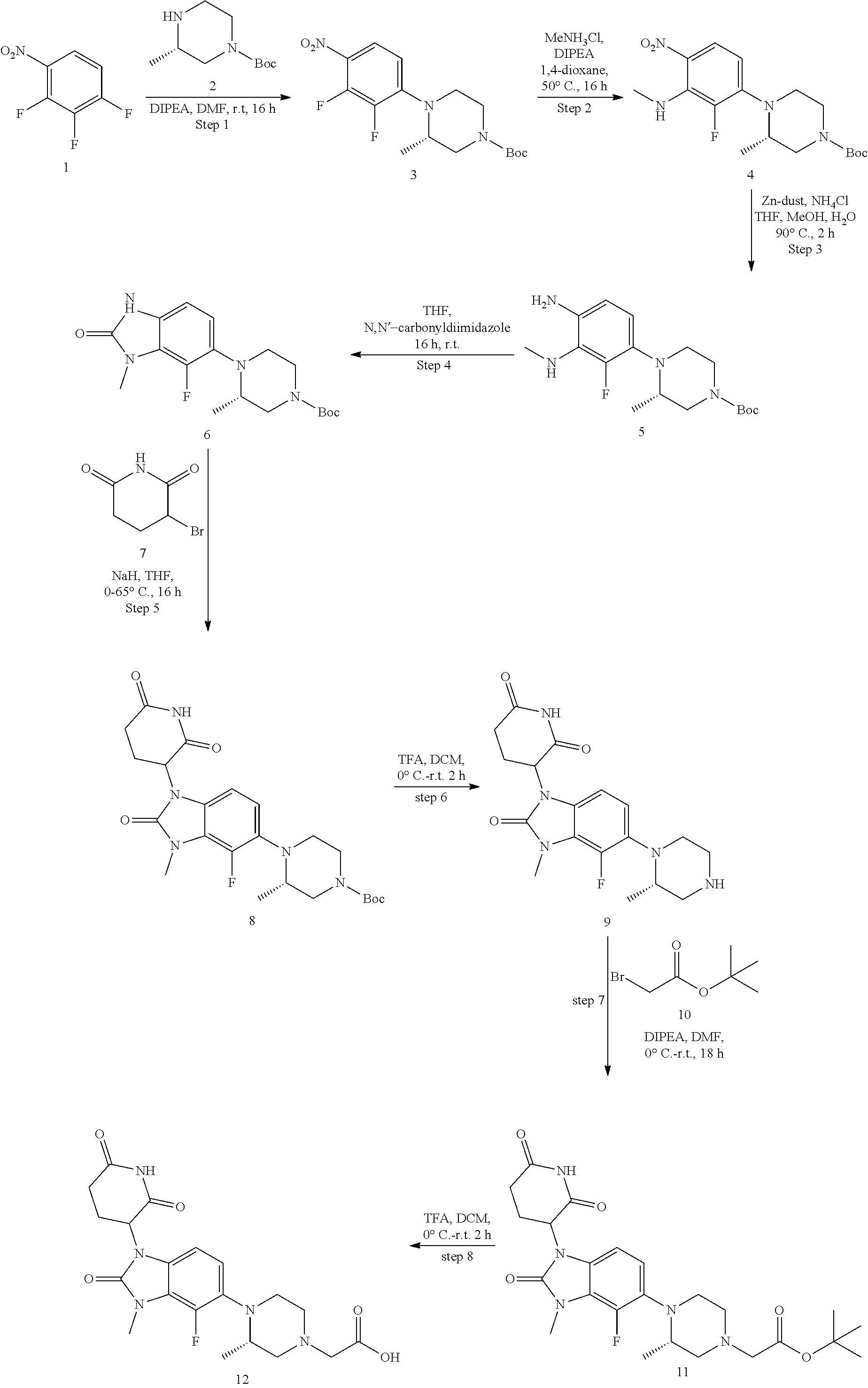

### Step 1: tert-butyl (3S)-4-(2,3-difluoro-4-nitro-phenyl)-3-methyl-piperazine-1-carboxylate (3)

To a 100 mL single neck round bottom flask containing a well-stirred solution of tert-butyl (3S)-3-methylpiperazine-1-carboxylate (2, 5.65 g, 28.24 mmol) in DMF (20 mL), was added N,N-diisopropylethylamine (4.01 g, 31.06 mmol, 5.41 mL) drop-wise and stirred for 10 min. Subsequently, a solution of 1,2,3-trifluoro-4-nitro-benzene (1, 5 g, 28.24 mmol, 3.25 mL) in DMF (5 mL) was added drop-wise and stirred at ambient temperature for 16 h. The reaction mixture was diluted with water (100 mL) and extracted into EtOAc (2×150 mL). The combined EtOAc layer was washed with water (3×100 mL), brine solution (100 mL), dried over sodium sulfate, filtered, evaporated solvent under reduced pressure to afford tert-butyl (3S)-4-(2,3-difluoro-4-nitro-phenyl)-3-methyl-piperazine-1-carboxylate (3, 9.5 g, 20.20 mmol, 72% yield, 76% purity) as a yellow liquid.

LCMS (ES+): m/z 302.2 [M−tBu+H]⁺

### Step 2: tert-butyl (3S)-4-[2-fluoro-3-(methylamino)-4-nitro-phenyl]-3-methyl-piperazine-1-carboxylate (4)

To a 250 mL pressure tube containing a solution of tert-butyl (3S)-4-(2,3-difluoro-4-nitro-phenyl)-3-methyl-piperazine-1-carboxylate (3, 9.5 g, 20.20 mmol) in 1,4-dioxane (80 mL) was added methylamine hydrochloride (2.05 g, 30.31 mmol), followed by N,N-diisopropylethylamine (7.83 g, 60.61 mmol, 10.56 mL) and stirred at 90° C. for 16 h. The reaction mixture was diluted with water (100 mL), extracted into EtOAc (3×150 mL), washed with brine solution (100 mL), dried over sodium sulfate, filtered, and concentrated under reduced pressure to afford tert-butyl (3S)-4-[2-fluoro-3-(methylamino)-4-nitro-phenyl]-3-methyl-piperazine-1-carboxylate (4, 8 g, 17.81 mmol, 88% yield, 82% purity) as a yellow liquid. The material was used in the next step without further purification.

LCMS (ES+): m/z 369.2[M+H]⁺

### Step 3: tert-butyl (3S)-4-[4-amino-2-fluoro-3-(methylamino)phenyl]-3-methyl-piperazine-1-carboxylate (5)

To a 500 mL single neck round bottom flask containing a well-stirred solution of tert-butyl (3S)-4-[2-fluoro-3-(methylamino)-4-nitro-phenyl]-3-methyl-piperazine-1-carboxylate (4, 8 g, 17.81 mmol) in a mixture of THF (40 mL), methanol (80 mL) and water (40 mL), was added Zinc dust (5.82 g, 89.03 mmol), followed by ammonium chloride (4.76 g, 89.03 mmol) and heated at 90° C. for 2 h. The reaction mixture was diluted with water (50 mL) and DCM (300 mL) and filtered through Celite. The layers were separated, the DCM layer was washed with water (2×100 mL), brine solution (100 mL), dried over sodium sulfate, filtered, and solvent removed to afford tert-butyl (3S)-4-[4-amino-2-fluoro-3-(methylamino)phenyl]-3-methyl-piperazine-1-carboxylate (5, 7 g, 12.82 mmol, 72% yield, 62% purity) as a red sticky solid. The material was used in the next step without further purification.

LCMS (ES+): m/z 339.2 [M+H]⁺

### Step 4: tert-butyl (3S)-4-(4-fluoro-3-methyl-2-oxo-1H-benzimidazol-5-yl)-3-methyl-piperazine-1-carboxylate (6)

To a 250 mL single neck round bottom flask containing a well-stirred solution of tert-butyl (3S)-4-[4-amino-2-fluoro-3-(methylamino)phenyl]-3-methyl-piperazine-1-carboxylate (5, 7 g, 12.82 mmol) in THF (100 mL), was added N,N′-carbonyldiimidazole (6.24 g, 38.47 mmol). After 16 h, the reaction mixture was concentrated and purified by flash silicagel column chromatography (230-400 mesh silica gel; 75% EtOAc in pet-ether) to afford tert-butyl (3S)-4-(4-fluoro-3-methyl-2-oxo-1H-benzimidazol-5-yl)-3-methyl-piperazine-1-carboxylate (6, 4 g, 10.43 mmol, 81% yield) as a red solid.

LCMS (ES+): m/z 365.2 [M+H]⁺

### Step 5: tert-butyl (3S)-4-[1-(2,6-dioxo-3-piperidyl)-4-fluoro-3-methyl-2-oxo-benzimidazol-5-yl]-3-methyl-piperazine-1-carboxylate (8)

To a 1 L three-neck round bottom flask containing a well-stirred solution of tert-butyl (3S)-4-(4-fluoro-3-methyl-2-oxo-1H-benzimidazol-5-yl)-3-methyl-piperazine-1-carboxylate (6, 2 g, 5.21 mmol) in THF (250 mL), was added sodium hydride (60% in mineral oil) (2.09 g, 52.14 mmol) at 0° C. in three portions over 5 min interval and stirred at ambient temperature for 1 h. Then a solution of 3-bromopiperidine-2,6-dione (7, 4.00 g, 20.86 mmol) in THF (40 mL) was added drop-wise at 0° C. and stirred the reaction mixture at 65° C. for 16 h. The reaction was quenched with ammonium chloride solution (25 mL), diluted with water (10 mL) at 0° C., extracted into EtOAc (2×200 mL). The combined EtOAc layer was washed with brine solution (100 mL), dried over sodium sulfate, filtered, concentrated under reduced pressure. The crude compound was purified by flash silica gel flash column chromatography (230-400 mesh silica gel; 70% EtOAc in pet-ether) to obtain tert-butyl (3S)-4-[1-(2,6-dioxo-3-piperidyl)-4-fluoro-3-methyl-2-oxo-benzimidazol-5-yl]-3-methyl-piperazine-1-carboxylate (8, 1.65 g, 3.12 mmol, 60% yield) as a pale brown solid.

LCMS (ES+): m/z 476.2 [M+H]⁺

### Step 6: 3-[4-fluoro-3-methyl-5-[(2S)-2-methylpiperazin-1-yl]-2-oxo-benzimidazol-1-yl]piperidine-2,6-dione (9)

To a 100 mL single neck round bottom flask containing a well-stirred solution of tert-butyl (3S)-4-[1-(2,6-dioxo-3-piperidyl)-4-fluoro-3-methyl-2-oxo-benzimidazol-5-yl]-3-methyl-piperazine-1-carboxylate (8, 1.2 g, 2.27 mmol) in DCM (5 mL), was added trifluoroacetic acid (5.07 g, 44.50 mmol, 3.43 mL) at 0° C. and stirred the reaction mixture at ambient temperature for 2 h. The reaction mixture was concentrated under reduced pressure, co-distilled with toluene (2×15 mL), triturated with MTBE (30 mL), dried under reduced pressure to obtain 3-[4-fluoro-3-methyl-5-[(2S)-2-methylpiperazin-1-yl]-2-oxo-benzimidazol-1-yl]piperidine-2,6-dione (9, 1.15 g, 2.16 mmol, 95% yield, TFA salt) as a brown solid.

LCMS (ES+): m/z 376.2 [M+H]⁺

### Step 7: tert-butyl 2-[(3S)-4-[1-(2,6-dioxo-3-piperidyl)-4-fluoro-3-methyl-2-oxo-benzimidazol-5-yl]-3-methyl-piperazin-1-yl]acetate (11)

To a 100 mL single neck round bottom flask containing a well-stirred solution of 3-[4-fluoro-3-methyl-5-[(2S)-2-methylpiperazin-1-yl]-2-oxo-benzimidazol-1-yl]piperidine-2,6-dione (9, 1.1 g, 2.07 mmol, TFA salt) in DMF (10 mL), was added N,N-diisopropylethylamine (801.73 mg, 6.20 mmol, 1.08 mL) and cooled to 0° C. Then a solution of tert-butyl 2-bromoacetate (10, 443.66 mg, 2.27 mmol, 333.58 μL) in DMF (2 mL) was added drop-wise and stirred the reaction mixture at ambient temperature for 18 h. The reaction was quenched with ice-water, extracted into EtOAc (2×50 mL), washed with water (3×30 mL) and brine solution (30 mL), dried over sodium sulfate, filtered, and concentrated under reduced pressure to obtain tert-butyl 2-[(3S)-4-[1-(2,6-dioxo-3-piperidyl)-4-fluoro-3-methyl-2-oxo-benzimidazol-5-yl]-3-methyl-piperazin-1-yl]acetate (11, 800 mg, 1.50 mmol, 73% yield) as a brown solid.

LCMS (ES+): m/z 490.2 [M+H]⁺

### Step 8: 2-[(3S)-4-[1-(2,6-dioxo-3-piperidyl)-4-fluoro-3-methyl-2-oxo-benzimidazol-5-yl]-3-methyl-piperazin-1-yl]acetic acid (12)

To a 50 mL single neck round bottom flask containing a well-stirred solution of tert-butyl 2-[(3S)-4-[1-(2,6-dioxo-3-piperidyl)-4-fluoro-3-methyl-2-oxo-benzimidazol-5-yl]-3-methyl-piperazin-1-yl]acetate (11, 800 mg, 1.50 mmol) in DCM (6 mL), was added trifluoroacetic acid (11.84 g, 103.84 mmol, 8 mL) at 0° C. and the reaction mixture was stirred at ambient temperature for 7 h. The reaction mixture was concentrated under reduced pressure, co-distilled with toluene (2×10 mL), triturated with MTBE (20 mL), dried under reduced pressure to obtain 2-[(3S)-4-[1-(2,6-dioxo-3-piperidyl)-4-fluoro-3-methyl-2-oxo-benzimidazol-5-yl]-3-methyl-piperazin-1-yl]acetic acid (12, 700 mg, 1.05 mmol, 70% yield, TFA salt) as a brown solid.

LCMS (ES+): m/z 434.2 [M+H]⁺

### 2-[(3R)-4-[1-(2,6-dioxo-3-piperidyl)-4-fluoro-3-methyl-2-oxo-benzimidazol-5-yl]-3-methyl-piperazin-1-yl]acetic acid (12)

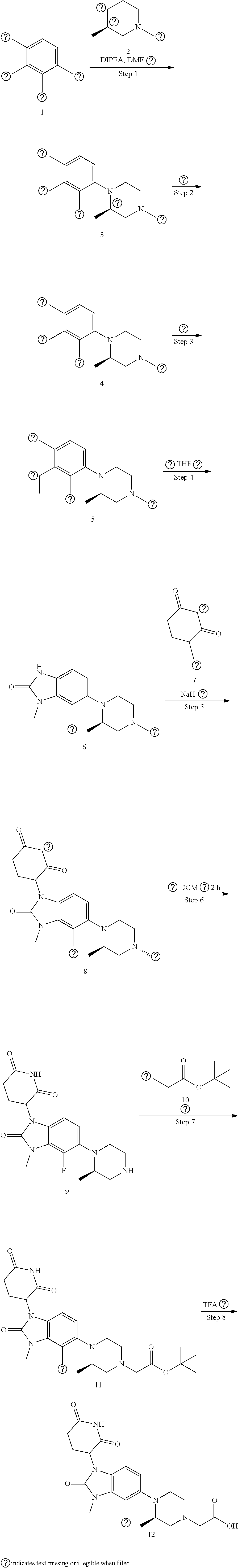

2-[(3R)-4-[1-(2,6-dioxo-3-piperidyl)-4-fluoro-3-methyl-2-oxo-benzimidazol-5-yl]-3-methyl-piperazin-1-yl]acetic acid (12, 200 mg, 313.71 μmol, TFA salt) was synthesized following the same eight step procedure as 2-[(3S)-4-[1-(2,6-dioxo-3-piperidyl)-4-fluoro-3-methyl-2-oxo-benzimidazol-5-yl]-3-methyl-piperazin-1-yl]acetic acid.

LCMS (ES+): m/z 434.2 [M+H]⁺

### 2-(3-(1-(2,6-dioxopiperidin-3-yl)-3-methyl-2-oxo-2,3-dihydro-1H-benzo[d]imidazol-5-yl)-1H-pyrazol-1-yl)acetic acid (5)

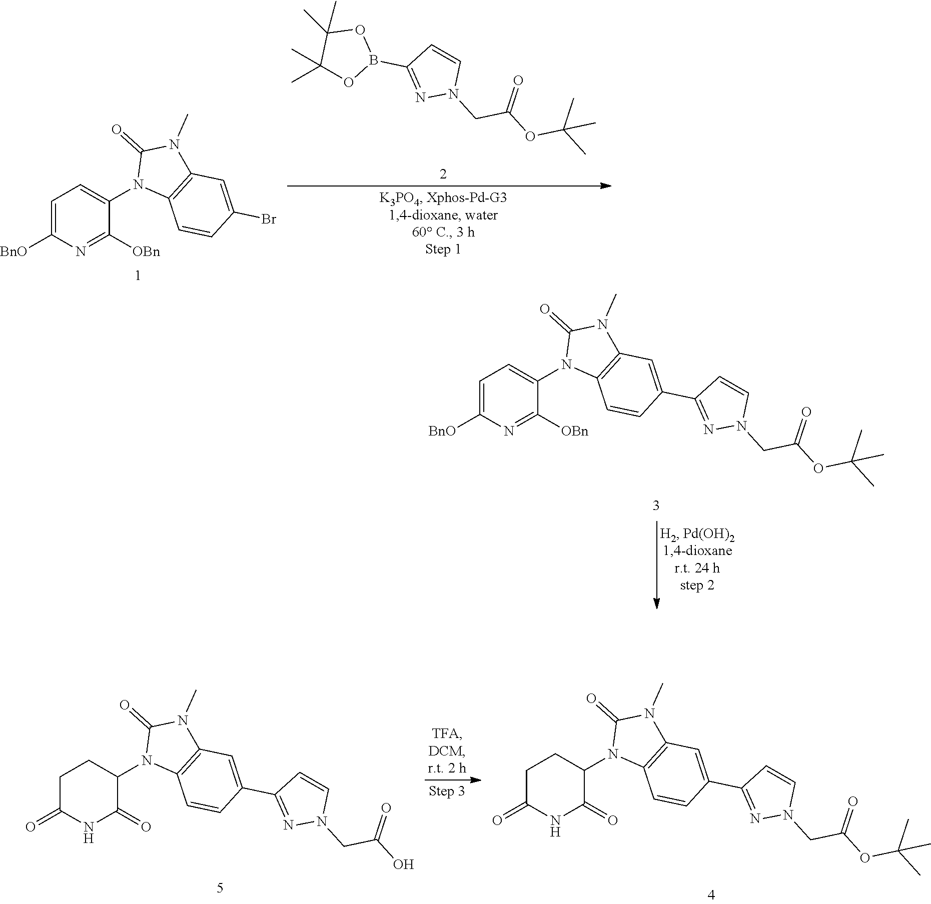 [SMILES: Cn1c(=O)n(-c2ccc(OCc3ccccc3)nc2OCc2ccccc2)c2ccc(Br)cc21 ; CC(C)(C)OC(=O)Cn1ccc(B2OC(C)(C)C(C)(C)O2)n1 ; Cn1c(=O)n(-c2ccc(OCc3ccccc3)nc2OCc2ccccc2)c2ccc(-c3ccn(CC(=O)OC(C)(C)C)n3)cc21 ; Cn1c(=O)n(C2CCC(=O)NC2=O)c2ccc(-c3ccn(CC(=O)O)n3)cc21 ; Cn1c(=O)n(C2CCC(=O)NC2=O)c2ccc(-c3ccn(CC(=O)OC(C)(C)C)n3)cc21]

### Step 1: tert-butyl 2-[3-[1-(2,6-dibenzyloxy-3-pyridyl)-3-methyl-2-oxo-benzimidazol-5-yl]pyrazol-1-yl]acetate (2)

Into a 100 mL pressure tube containing a well-stirred solution of 5-bromo-1-(2,6-dibenzyloxy-3-pyridyl)-3-methyl-benzimidazol-2-one (1, 1 g, 1.94 mmol) and tert-butyl 2-[3-(4,4,5,5-tetramethyl-1,3,2-dioxaborolan-2-yl)pyrazol-1-yl]acetate (2, 942.32 mg, 2.90 mmol) in 1,4-dioxane (8 mL) and water (0.2 mL) was added anhydrous potassium phosphate tribasic (1.23 g, 5.81 mmol). The reaction mixture was purged with nitrogen gas for 10 min. Then, X phos Pd G3 (245.75 mg, 290.48 μmol) was added and the reaction mixture was stirred at 60° C. for 2 h. The reaction mixture was filtered through Celite and washed with ethyl acetate (250 mL). The filtrate was concentrated under reduced pressure and purified by flash silica gel column chromatography (0-60% EtOAc in pet ether) to afford tert-butyl 2-[3-[1-(2,6-dibenzyloxy-3-pyridyl)-3-methyl-2-oxo-benzimidazol-5-yl]pyrazol-1-yl]acetate (850 mg, 1.31 mmol, 67% yield) as a pale yellow solid.

LCMS (ES+): m/z 618.2 [M+H]⁺

### Step 2: tert-butyl 2-[3-[1-(2,6-dioxo-3-piperidyl)-3-methyl-2-oxo-benzimidazol-5-yl]pyrazol-1-yl]acetate (4)

Into a 100 mL single neck round bottom flask containing a well-stirred solution of tert-butyl 2-[3-[1-(2,6-dibenzyloxy-3-pyridyl)-3-methyl-2-oxo-benzimidazol-5-yl]pyrazol-1-yl]acetate (3, 750 mg, 1.12 mmol) in anhydrous 1,4-dioxane (15 mL) was added 20 wt. % palladium hydroxide on carbon (784.40 mg, 1.12 mmol). The reaction mixture was stirred at room temperature under hydrogen atmosphere for 16 h. The reaction mixture was filtered through Celite and washed with 1,4-dioxane (200 mL). The filtrate was concentrated under reduced pressure and the residue was triturated with diethyl ether (20 mL), decanted and dried to obtain tert-butyl 2-[3-[1-(2,6-dioxo-3-piperidyl)-3-methyl-2-oxo-benzimidazol-5-yl]pyrazol-1-yl]acetate (4, 550 mg, 1.07 mmol, 96% yield)

UPLC (ES+): m/z 440.6 [M+H]⁺

### Step 3: 2-(3-(1-(2,6-dioxopiperidin-3-yl)-3-methyl-2-oxo-2,3-dihydro-1H-benzo[d]imidazol-5-yl)-1H-pyrazol-1-yl)acetic acid (5)

Into a 100 mL single round bottom flask containing stirred solution of tert-butyl 2-[3-[1-(2,6-dioxo-3-piperidyl)-3-methyl-2-oxo-benzimidazol-5-yl]pyrazol-1-yl]acetate (4, 500 mg, 972.09 μmol) in DCM (6 mL) was added TFA (110.84 mg, 972.09 μmol, 74.89 μL). After 2 h, the volatiles were removed under reduced pressure and the residue washed with MTBE (2×50 mL) and dried to afford 2-(3-(1-(2,6-dioxopiperidin-3-yl)-3-methyl-2-oxo-2,3-dihydro-1H-benzo[d]imidazol-5-yl)-1H-pyrazol-1-yl)acetic acid (5, 320 mg, 620.79 μmol, 70% yield, TFA salt) as an off-white solid.

LCMS (ES+): m/z 384.2 [M+H]⁺

### 2-[(3R)-4-[1-(2,6-dioxo-3-piperidyl)-6-fluoro-3-methyl-2-oxo-benzimidazol-5-yl]-3-methyl-piperazin-1-yl]acetic acid (12)

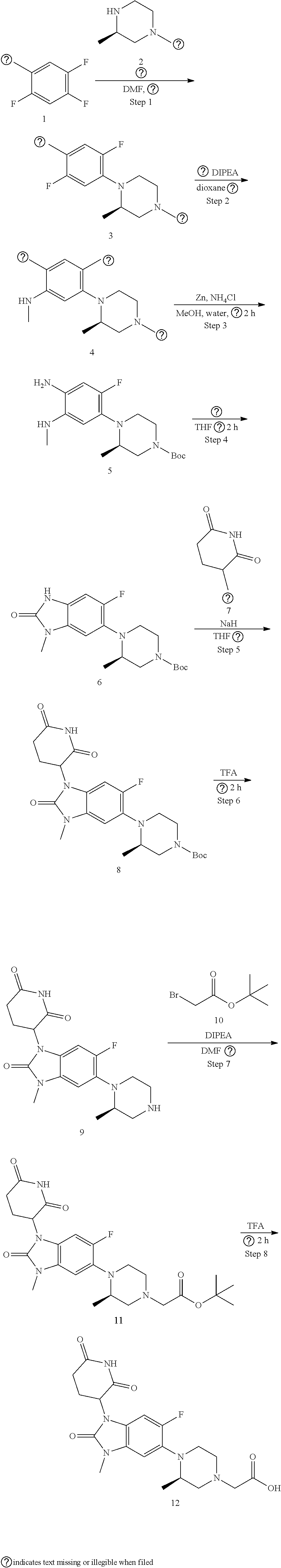

### Step 1: tert-butyl (3R)-4-(2,5-difluoro-4-nitro-phenyl)-3-methyl-piperazine-1-carboxylate (3)

Into a 100 mL single neck round bottom flask containing a well-stirred solution of tert-butyl (R)-3-methylpiperazine-1-carboxylate (2, 5.0 g, 28.24 mmol) in DMF (20 mL) was added N,N-Diisopropylethylamine (3.55 g, 27.46 mmol, 4.78 mL) drop-wise and stirred for 10 min. Subsequently, 1,2,4-trifluoro-5-nitrobenzene (1, 4.42 g, 24.97 mmol, 2.87 mL) in DMF (5 mL). After 18 h the reaction mixture was poured into water (100 mL) and extracted with EtOAc (2×200 mL). The combined organic layers were washed with water (3×100 mL), brine (100 mL), dried over sodium sulfate, filtered and solvent removed to afford tert-butyl (3R)-4-(2,5-difluoro-4-nitro-phenyl)-3-methyl-piperazine-1-carboxylate (3, 6.9 g, 19.06 mmol, 76% yield) as a yellow solid.

LCMS (ES+): m/z 302.2 [M−tBu+H]⁺

### Step 2: tert-butyl (3R)-4-[2-fluoro-5-(methylamino)-4-nitro-phenyl]-3-methyl-piperazine-1-carboxylate (4)

Into a 250 mL sealed-tube containing a well-stirred solution of tert-butyl (3R)-4-(2,5-difluoro-4-nitro-phenyl)-3-methyl-piperazine-1-carboxylate (3, 8.71 g, 24.07 mmol) and methanamine hydrochloride (2.27 g, 33.69 mmol) in 1,4-dioxane (100 mL) was added N,N-diisopropylethylamine (9.33 g, 72.20 mmol, 12.58 mL) and the mixture was heated at 90° C. for 16 h. The reaction mixture was poured into water (150 mL) and extracted with EtOAc (3×200 mL). The combined organic layers were washed with brine (100 mL), dried over sodium sulfate, filtered, and concentrated to afford the crude material that was purified by silica gel (230-400) flash column chromatography (45% ethyl acetate in Pet ether) to obtain tert-butyl (3R)-4-[2-fluoro-5-(methylamino)-4-nitro-phenyl]-3-methyl-piperazine-1-carboxylate (4, 5.2 g, 13.16 mmol, 55% yield).

LCMS (ES+): m/z 369.2 [M+H]⁺

### Step 3: tert-butyl (3R)-4-[4-amino-2-fluoro-5-(methylamino)phenyl]-3-methyl-piperazine-1-carboxylate (5)

Into a 100 mL single neck round bottom flask containing a well-stirred solution of tert-butyl (3R)-4-[2-fluoro-5-(methylamino)-4-nitro-phenyl]-3-methyl-piperazine-1-carboxylate (4, 4.20 g, 10.63 mmol) in a mixture of THF (20 mL), methanol (40 mL) and water (20 mL) were added zinc powder (325 mesh high grade material) (3.48 g, 53.17 mmol) and ammonium chloride (2.84 g, 53.17 mmol) and the resultant mixture was stirred at 90° C. for 3 h. The reaction mixture was filtered through Celite and washed with EtOAc. The filtrate was washed with water (50 mL), dried over sodium sulfate, filtered and concentrated to afford the crude tert-butyl (3R)-4-[4-amino-2-fluoro-5-(methylamino)phenyl]-3-methyl-piperazine-1-carboxylate (5, 3.7 g, 10.34 mmol, 97% yield) as a red sticky solid.

LCMS (ES+): m/z 339.2 [M+H]⁺

### Step 4: tert-butyl (3R)-4-(6-fluoro-3-methyl-2-oxo-1H-benzimidazol-5-yl)-3-methyl-piperazine-1-carboxylate (6)

Into a 100 mL single neck round bottom flask containing a well-stirred solution of tert-butyl (3R)-4-[4-amino-2-fluoro-5-(methylamino)phenyl]-3-methyl-piperazine-1-carboxylate (5, 3.75 g, 10.24 mmol) in THF (150 mL) was added N,N′-Carbonyldiimidazole (4.98 g, 30.72 mmol) and the resultant mixture was stirred at room temperature for 2 h. The reaction mixture was concentrated and purified by silica gel flash column chromatography (230-400 mesh silica gel; 60-70% EtOAc in pet-ether) to obtain tert-butyl (3R)-4-(6-fluoro-3-methyl-2-oxo-1H-benzimidazol-5-yl)-3-methyl-piperazine-1-carboxylate (6, 3.2 g, 8.31 mmol, 81% yield) as a red solid.

LCMS (ES+): m/z 365.2 [M+H]⁺

### Step 5: tert-butyl (3R)-4-[1-(2,6-dioxo-3-piperidyl)-6-fluoro-3-methyl-2-oxo-benzimidazol-5-yl]-3-methyl-piperazine-1-carboxylate (8)

Into a 250 mL two neck round bottom flask containing a well-stirred solution of tert-butyl (3R)-4-(6-fluoro-3-methyl-2-oxo-1H-benzimidazol-5-yl)-3-methyl-piperazine-1-carboxylate (6, 1.06 g, 2.74 mmol) in THF (20 mL) was added sodium hydride (60% dispersion in mineral oil) (841.17 mg, 21.95 mmol) at 0° C. in three portions over 5 min and the resultant suspension was stirred at room temperature for 1 h. To this, 3-bromopiperidine-2,6-dione (7, 2.15 g, 10.98 mmol) in THF (10 mL) was added dropwise at 0° C. and the reaction mixture was heated at 65° C. for 16 h. The reaction was quenched with ammonium chloride solution (125 mL) at 0° C. and extracted with EtOAc (2×200 mL). The combined organic layers were washed with brine (100 mL), dried over sodium sulfate, filtered and concentrated under reduced pressure and purified by silica gel flash column chromatography (230-400 mesh silica gel; 60-70% EtOAc in pet-ether) to afford tert-butyl (3R)-4-[1-(2,6-dioxo-3-piperidyl)-6-fluoro-3-methyl-2-oxo-benzimidazol-5-yl]-3-methyl-piperazine-1-carboxylate (8, 0.82 g, 1.32 mmol, 48% yield) as a white solid.

LCMS (ES+): m/z 476.2 [M+H]⁺

### Step 6: 3-[6-fluoro-3-methyl-5-[(2R)-2-methylpiperazin-1-yl]-2-oxo-benzimidazol-1-yl]piperidine-2,6-dione (9)

Into a 50 mL single neck round bottom flask containing a well-stirred solution of tert-butyl (3R)-4-[1-(2,6-dioxo-3-piperidyl)-6-fluoro-3-methyl-2-oxo-benzimidazol-5-yl]-3-methyl-piperazine-1-carboxylate (8, 823.05 mg, 1.51 mmol) in DCM (10 mL) was added trifluoroacetic acid (1.73 g, 15.14 mmol, 1.17 mL) at 0° C. and the resultant mixture was stirred at room temperature for 2 h. The reaction mixture was concentrated under reduced pressure and triturated with MTBE (30 mL) to obtain 3-[6-fluoro-3-methyl-5-[(2R)-2-methylpiperazin-1-yl]-2-oxo-benzimidazol-1-yl]piperidine-2,6-dione (9, 0.73 g, 1.49 mmol, 98% yield, TFA salt) as a brown solid.

LCMS (ES+): m/z 376.2 [M+H]⁺

### Step 7: tert-butyl 2-[(3R)-4-[1-(2,6-dioxo-3-piperidyl)-6-fluoro-3-methyl-2-oxo-benzimidazol-5-yl]-3-methyl-piperazin-1-yl]acetate (11)

Into a 50 mL single neck round bottom flask containing a well-stirred solution of 3-[6-fluoro-3-methyl-5-[(2R)-2-methylpiperazin-1-yl]-2-oxo-benzimidazol-1-yl]piperidine-2,6-dione (9, 583.21 mg, 1.19 mmol, TFA salt) in DMF (3 mL) at 0° C. was added N,N-diisopropylethylamine (459.49 mg, 3.56 mmol, 619.26 μL) and tert-butyl 2-bromoacetate (10, 231.15 mg, 1.19 mmol, 173.80 μL) and the resultant mixture was stirred at room temperature for 1 h. The reaction mixture was poured into ice-cold water and extracted with EtOAc (2×20 mL). The combined organic layers were washed with water (3×10 mL) and brine solution (10 mL), dried over sodium sulfate, filtered and concentrated to obtain tert-butyl 2-[(3R)-4-[1-(2,6-dioxo-3-piperidyl)-6-fluoro-3-methyl-2-oxo-benzimidazol-5-yl]-3-methyl-piperazin-1-yl]acetate (11, 560 mg, 884.83 μmol, 75% yield, 77% purity).

LCMS (ES+): m/z 490.1 [M+H]⁺

### Step 8: 2-[(3R)-4-[1-(2,6-dioxo-3-piperidyl)-6-fluoro-3-methyl-2-oxo-benzimidazol-5-yl]-3-methyl-piperazin-1-yl]acetic acid (12)

Into a 50 mL single neck round bottom flask containing a well-stirred solution tert-butyl 2-[(3R)-4-[1-(2,6-dioxo-3-piperidyl)-6-fluoro-3-methyl-2-oxo-benzimidazol-5-yl]-3-methyl-piperazin-1-yl]acetate (11, 662 mg, 1.14 mmol) in DCM (5 mL) was added trifluoroacetic acid (2.61 g, 22.88 mmol, 1.76 mL) at 0° C. and the resultant mixture was stirred at room temperature for 2 h. The reaction mixture was concentrated under reduced pressure and triturated with MTBE (20 mL) to afford 2-[(3R)-4-[1-(2,6-dioxo-3-piperidyl)-6-fluoro-3-methyl-2-oxo-benzimidazol-5-yl]-3-methyl-piperazin-1-yl]acetic acid (12, 0.54 g, 929.66 μmol, 81% yield, TFA salt) as a white solid.

LCMS (ES+): m/z 434.2 [M+H]⁺

### 2-[(3R)-3-[1-(2,6-dioxo-3-piperidyl)-3-methyl-2-oxo-benzimidazol-5-yl]-1-piperidyl]acetic acid (9a)

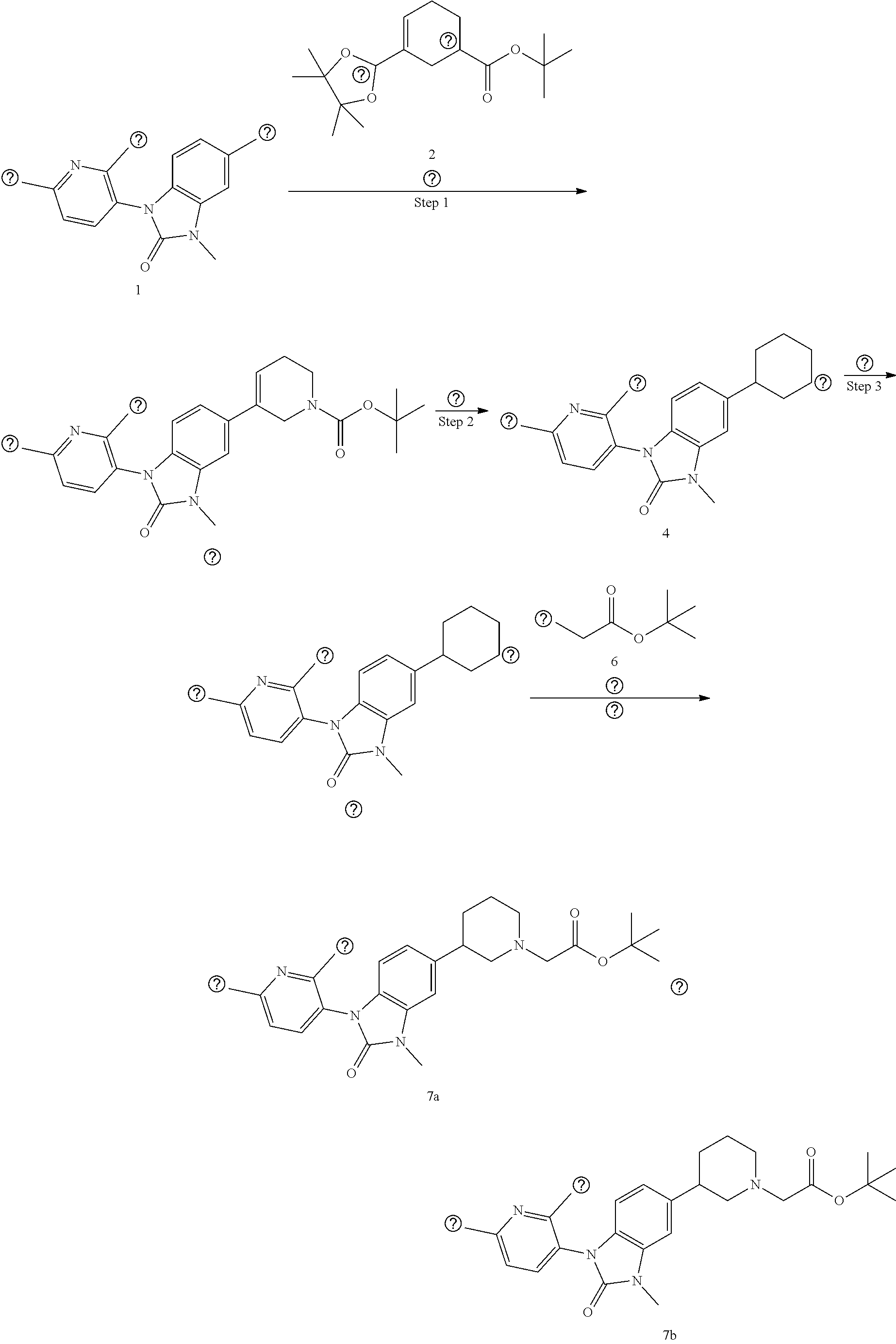

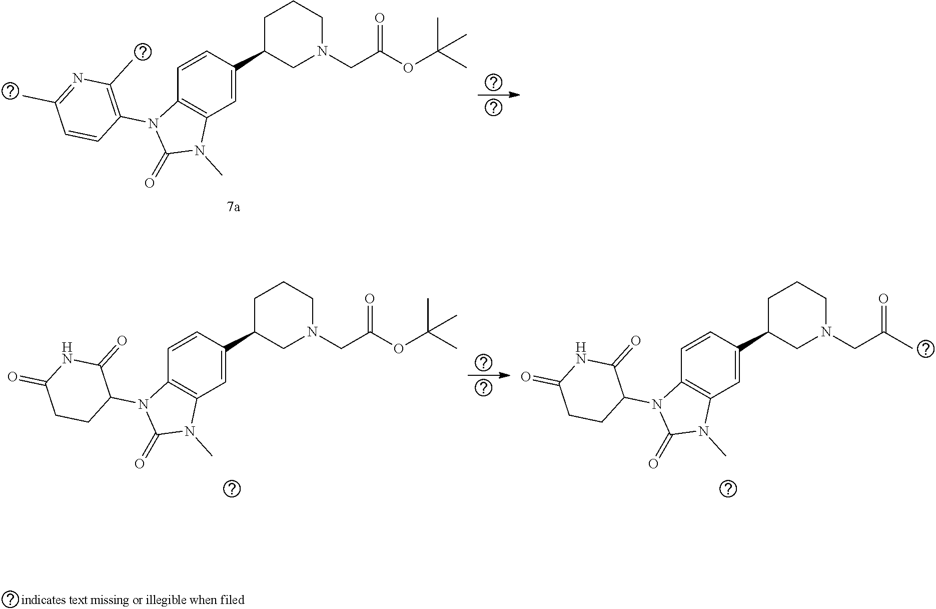 [SMILES: Cc1ccc(-n2c(=O)n(C)c3cc([C@H]4CCCN(CC(=O)OC(C)(C)C)C4)ccc32)c(C)n1 ; Cn1c(=O)n(C2CCC(=O)NC2=O)c2ccc([C@H]3CCCN(CC(=O)OC(C)(C)C)C3)cc21 ; CC(=O)CN1CCC[C@H](c2ccc3c(c2)n(C)c(=O)n3C2CCC(=O)NC2=O)C1]

### Step 1: tert-butyl 5-[1-(2,6-dibenzyloxy-3-pyridyl)-3-methyl-2-oxo-benzimidazol-5-yl]-3,6-dihydro-2H-pyridine-1-carboxylate (3)

Into a 250 mL pressure tube-1,2-diame containing a well-stirred solution of tert-butyl 5-(4,4,5,5-tetramethyl-1,3,2-dioxaborolan-2-yl)-3,6-dihydro-2H-pyridine-1-carboxylate (2, 1.92 g, 6.20 mmol) and 5-bromo-1-(2,6-dibenzyloxy-3-pyridyl)-3-methyl-benzimidazol-2-one (1, 3 g, 5.64 mmol) in a mixture of water (15 mL) and 1,4-dioxane (40 mL), was added Cesium carbonate (5.51 g, 16.91 mmol). The reaction mixture was degassed with nitrogen for 10 min. Subsequently, PdCl₂(dtbpf) (183.64 mg, 281.77 μmol) was added and degassed for another 5 min. The reaction mixture was stirred at 90° C. for 8 h. The reaction mixture was cooled to ambient temperature, diluted with EtOAc (150 mL) and filtered through Celite and washed with EtOAc (150 mL). The combined EtOAc layer was dried over sodium sulfate, concentrated and purified by flash silica gel flash column chromatography (30-40% EtOAc in Pet. ether) to obtain tert-butyl 5-[1-(2,6-dibenzyloxy-3-pyridyl)-3-methyl-2-oxo-benzimidazol-5-yl]-3,6-dihydro-2H-pyridine-1l-carboxylate (3, 3.5 g, 5.43 mmol, 96% yield) as a brown sticky solid.

LCMS (ES+): m/z 619.2 [M+H]⁺

### Step 2: tert-butyl 3-[1-(2,6-dibenzyloxy-3-pyridyl)-3-methyl-2-oxo-benzimidazol-5-yl]piperidine-1-carboxylate (4)

Into a 250 mL miniclave vessel (steel pressure reactor) containing a solution of tert-butyl 5-[1-(2,6-dibenzyloxy-3-pyridyl)-3-methyl-2-oxo-benzimidazol-5-yl]-3,6-dihydro-2H-pyridine-1-carboxylate (3, 1.5 g, 2.33 mmol) in toluene (40 mL) were added acetic acid (838.59 mg, 13.96 mmol, 798.65 μL) and 5 wt. % palladium on carbon (371.52 mg, 174.55 μmol). Subsequently, sodium borohydride (880.52 mg, 23.28 mmol) was added in two portions. After 8 h, the reaction mixture was filtered through Celite and washed with a mixture of THF (350 mL) and toluene (150 mL), followed by a mixture of EtOAc (350 mL), ACN (150 mL). The solvent was removed under reduced pressure and the material was purified by flash silica gel column chromatography (45% EtOAc in pet ether) to obtain tert-butyl 3-[1-(2,6-dibenzyloxy-3-pyridyl)-3-methyl-2-oxo-benzimidazol-5-yl]piperidine-1-carboxylate (4, 1 g, 1.48 mmol, 64% yield) as a brown sticky solid.

LCMS (ES+): m/z 621.2 [M+H]⁺

### Step 3: 1-(2,6-dibenzyloxy-3-pyridyl)-3-methyl-5-(3-piperidyl)benzimidazol-2-one (5)

Into a 100 mL single neck round bottom flask containing a well-stirred solution of tert-butyl 3-[1-(2,6-dibenzyloxy-3-pyridyl)-3-methyl-2-oxo-benzimidazol-5-yl]piperidine-1-carboxylate (4, 1 g, 1.48 mmol) in DCM (7 mL) was added trifluoroacetic acid (1.013 g, 8.9 mmol, 685.12 μL) at 0° C. The reaction mixture was stirred at ambient temperature for 4 h. The reaction mixture was concentrated under reduced pressure, co-distilled with toluene (2×10 mL) and dried to obtain 1-(2,6-dibenzyloxy-3-pyridyl)-3-methyl-5-(3-piperidyl)benzimidazol-2-one (5, 1 g, 1.43 mmol, 97% yield, TFA salt) as a brown sticky solid.

LCMS (ES+): m/z 521.2 [M+H]⁺

### Step 4: tert-butyl 2-[(3R)-3-[1-(2,6-dibenzyloxy-3-pyridyl)-3-methyl-2-oxo-benzimidazol-5-yl]-1-piperidyl]acetate (7a) and tert-butyl 2-[(3S)-3-[1-(2,6-dibenzyloxy-3-pyridyl)-3-methyl-2-oxo-benzimidazol-5-yl]-1-piperidyl]acetate (7b)

Into a 100 mL single neck round bottom flask containing a well-stirred solution of 1-(2,6-dibenzyloxy-3-pyridyl)-3-methyl-5-(3-piperidyl)benzimidazol-2-one (5, 1 g, 1.43 mmol, TFA salt) in DMF (8 mL) was added N,N-diisopropylethylamine (926.57 mg, 7.17 mmol, 1.25 mL). Subsequently, a solution of tert-butyl 2-bromoacetate (6, 279.68 mg, 1.43 mmol, 210.28 μL) in DMF (1 mL) was added. The reaction mixture was stirred at ambient temperature under nitrogen atmosphere for 2 h. The reaction mixture was concentrated under reduced pressure and purified by reverse phase prep HPLC [Purification method: Column: XSelect C18 (19×250) mm, 5 micron; Mobile phase A: 0.1% TFA in MQ Water; Mobile phase B: MeCN] to afford the racemic compound (1 g with purity 99%) that was subjected to SFC Chiral purification.

### Chiral Separation:

The enantiomers were separated by chiral SFC.

Method details: Column Name: Lux A1; Co-solvent: 40% and Co-solvent Name: 0.5% Isopropyl Amine in IPA; Outlet Pressure: 100 bar; Temperature: 35° C.

The fast eluted isomer: tert-butyl 2-[(3R)-3-[1-(2,6-dibenzyloxy-3-pyridyl)-3-methyl-2-oxo-benzimidazol-5-yl]-1-piperidyl]acetate (7a, 350 mg, 540.36 μmol, 38% yield) (RT: 3.18 min), arbitrarily assigned as R-isomer was isolated and re-purified by flash silicagel column chromatography (230-400 mesh silica gel, 70% EtOAc in pet ether) to obtain compound as pale brown sticky solid.

LCMS (ES+): m/z 635.2 [M+H]⁺

Late eluted isomer: tert-butyl 2-[(3S)-3-[1-(2,6-dibenzyloxy-3-pyridyl)-3-methyl-2-oxo-benzimidazol-5-yl]-1-piperidyl]acetate (7b, 300 mg, 467.89 μmol, 33% yield) (RT: 3.77 min) arbitrarily assigned as S-isomer was isolated and re-purified by Silicagel flash column chromatography (230-400 mesh silica gel, 70% EtOAc in pet ether) to obtain compound as pale brown sticky solid.

LCMS (ES+): m/z 635.2 [M+H]⁺

### Step 5a: tert-butyl 2-[(3R)-3-[1-(2,6-dioxo-3-piperidyl)-3-methyl-2-oxo-benzimidazol-5-yl]-1-piperidyl]acetate (8a)

Into a 100 mL single neck round bottom flask containing a well-stirred solution of tert-butyl 2-[(3R)-3-[1-(2,6-dibenzyloxy-3-pyridyl)-3-methyl-2-oxo-benzimidazol-5-yl]-1-piperidyl]acetate (7a, 700 mg, 1.08 mmol) in 1,4-dioxane (10 mL) and DMF (3 mL) were added acetic acid (682.10 mg, 11.36 mmol, 649.62 μL) and 20 wt. % palladium hydroxide on carbon (758.85 mg, 1.08 mmol, 20% purity). The suspension was stirred under hydrogen atmosphere for 16 h at ambient temperature. The reaction mixture was filtered through Celite and washed with a mixture of 1,4-dioxane (200 mL)/DMF (200 mL) and a mixture of THF (150 mL)/MeCN (150 mL). The combined filtrate was concentrated under reduced pressure and purified by reverse phase column chromatography [Redisep C18 120 g, Mobile phase A: 0.1% Formic acid in MQ Water; Mobile phase B: MeCN] to obtain tert-butyl 2-[(3R)-3-[1-(2,6-dioxo-3-piperidyl)-3-methyl-2-oxo-benzimidazol-5-yl]-1-piperidyl]acetate (8a, 180 mg, 343.84 μmol, 32% yield, Formic acid salt) as a pale brown solid.

LCMS (ES+): m/z 457.2 [M+H]⁺

### Step 6a: 2-[(3R)-3-[1-(2,6-dioxo-3-piperidyl)-3-methyl-2-oxo-benzimidazol-5-yl]-1-piperidyl]acetic acid (9a)

Into a 50 mL single neck round bottom flask containing a solution of tert-butyl 2-[(3R)-3-[1-(2,6-dioxo-3-piperidyl)-3-methyl-2-oxo-benzimidazol-5-yl]-1-piperidyl]acetate (8a, 180 mg, 343.84 μmol, Formic acid salt) in DCM (3 mL), was added trifluoroacetic acid (2.96 g, 25.96 mmol, 2 mL) drop-wise at 0° C. The reaction mixture was stirred at ambient temperature under nitrogen atmosphere for 4 h. The reaction mixture was concentrated under vacuum, triturated with MTBE (2×5 mL), filtered and dried under reduced pressure to obtain 2-[(3R)-3-[1-(2,6-dioxo-3-piperidyl)-3-methyl-2-oxo-benzimidazol-5-yl]-1-piperidyl]acetic acid (9a, 170 mg, 318.29 μmol, 93% yield, TFA salt) as a red solid.

LCMS (ES+): m/z 401.2 [M+H]⁺

### 2-[(3S)-3-[1-(2,6-dioxo-3-piperidyl)-3-methyl-2-oxo-benzimidazol-5-yl]-1-piperidyl]acetic acid (3)

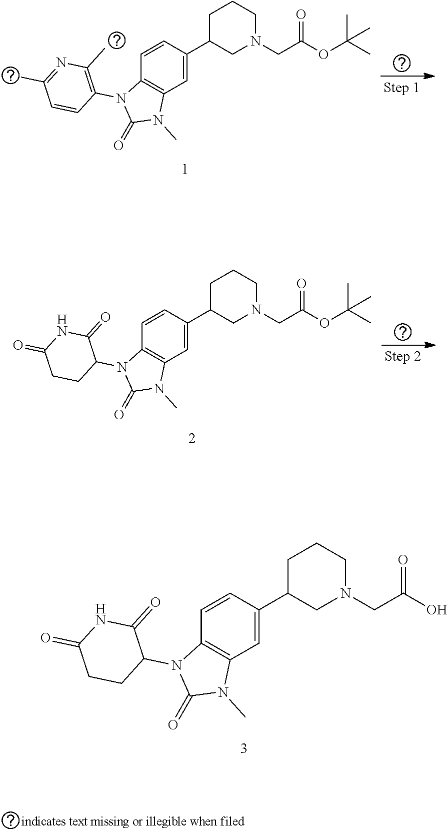 [SMILES: Cc1ccc(-n2c(=O)n(C)c3cc(C4CCCN(CC(=O)OC(C)(C)C)C4)ccc32)c(C)n1 ; Cn1c(=O)n(C2CCC(=O)NC2=O)c2ccc(C3CCCN(CC(=O)OC(C)(C)C)C3)cc21 ; Cn1c(=O)n(C2CCC(=O)NC2=O)c2ccc(C3CCCN(CC(=O)O)C3)cc21]

2-[(3S)-3-[1-(2,6-dioxo-3-piperidyl)-3-methyl-2-oxo-benzimidazol-5-yl]-1-piperidyl]acetic acid (3, 150 mg, 273.76 μmol, TFA salt) was synthesized following the same two step procedure as 2-[(3R)-3-[1-(2,6-dioxo-3-piperidyl)-3-methyl-2-oxo-benzimidazol-5-yl]-1-piperidyl]acetic acid.

LCMS (ES+): m/z 401.2 [M+H]⁺

### 2-[1-[1-(2,6-dioxo-3-piperidyl)-3-methyl-2-oxo-benzimidazol-5-yl] pyrazol-4-yl]acetic acid (5)

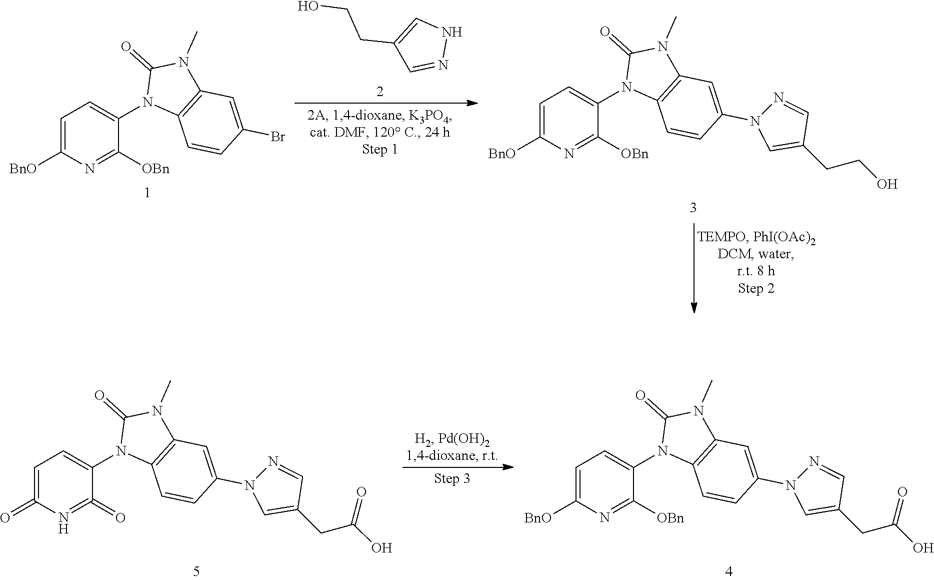

### Step 1: 1-(2,6-dibenzyloxy-3-pyridyl)-5-[4-(2-hydroxyethyl) pyrazol-1-yl]-3-methyl-benzimidazol-2-one (3)

Into a 100 mL pressure tube containing a well-stirred solution of 5-bromo-1-(2,6-dibenzyloxy-3-pyridyl)-3-methyl-benzimidazol-2-one (1, 1.5 g, 2.56 mmol) and 2-(1H-pyrazol-4-yl)ethanol (2, 573.26 mg, 5.11 mmol) in anhydrous DMF (20 mL) were added (1R,2R)-(−)-N,N′-dimethylcyclohexane-1,2-diamine (2A, 363.60 mg, 2.56 mmol), potassium carbonate (883.22 mg, 6.39 mmol) and copper (I) iodide (730.25 mg, 3.83 mmol, 129.94 uL). The reaction mixture was stirred at 120° C. for 16 h. The reaction mixture was passed through Celite and washed with EtOAc (200 mL). The filtrate was washed with brine solution (2×70 mL), dried over anhydrous Na₂SO₄, filtered and concentrated under reduced pressure. The material was purified by flash silica gel (230-400 mesh) column chromatography (100% EtOAc in pet ether) to afford 1-(2,6-dibenzyloxy-3-pyridyl)-5-[4-(2-hydroxyethyl)pyrazol-1-yl]-3-methyl-benzimidazol-2-one (3, 865 mg, 1.45 mmol, 57% yield) as brown color solid.

LCMS (ES+): m/z 548.2 [M+H]⁺

### Step 2: 2-[1-[1-(2,6-dibenzyloxy-3-pyridyl)-3-methyl-2-oxo-benzimidazol-5-yl] pyrazol-4-yl] acetic acid (4)

Into a 50 mL single neck round bottom flask containing a well-stirred solution of 1-(2,6-dibenzyloxy-3-pyridyl)-5-[4-(2-hydroxyethyl) pyrazol-1-yl]-3-methyl-benzimidazol-2-one (3, 300 mg, 499.69 μmol) in DCM (4 mL) and water (2 mL) were added TEMPO, (39.04 mg, 249.84 mol) and bis(acetoxy)iodobenzene (804.75 mg, 2.50 mmol). After 5 h, the reaction was quenched with aqueous Na₂S₂O₃ solution (10 mL). The organic layer was removed and the aqueous layer was acidified with 1.5N HCl and extracted with ethyl acetate (2×30 mL). The combined organic layer was dried over sodium sulfate, filtered, solvent removed and purified by reverse phase column chromatography [Silicycle C18 column; Mobile phase A: 0.1% Formic acid in water; Mobile phase B: MeCN] to afford 2-[1-[1-(2,6-dibenzyloxy-3-pyridyl)-3-methyl-2-oxo-benzimidazol-5-yl] pyrazol-4-yl] acetic acid (4, 184 mg, 270.91 μmol, 54% yield, formic acid salt) as a yellow solid.

LCMS (ES+): m/z 562.2 [M+H]⁺

### Step 3: 2-[1-[1-(2,6-dioxo-3-piperidyl)-3-methyl-2-oxo-benzimidazol-5-yl] pyrazol-4-yl]acetic acid (5)

Into a 50 mL single neck round bottom flask containing a well-stirred solution of 2-[1-[1-(2,6-dibenzyloxy-3-pyridyl)-3-methyl-2-oxo-benzimidazol-5-yl] pyrazol-4-yl]acetic acid (4, 353 mg, 479.60 μmol) in 1,4-dioxane (5 mL) was added 20 wt. % palladium hydroxide on carbon, (320 mg, 455.73 μmol). The suspension was stirred at room temperature under hydrogen bladder pressure for 16 h. The reaction mixture was filtered and washed with THF (50 mL), DMF (50 mL) and concentrated under reduced pressure. The material was purified by reverse phase prep HPLC [Purification method: Column: XSelect C18 (250×19) mm 5 micron; Mobile phase A: 0.1% Formic acid in water; Mobile phase B: MeCN] to afford 2-[1-[1-(2,6-dioxo-3-piperidyl)-3-methyl-2-oxo-benzimidazol-5-yl]pyrazol-4-yl]acetic acid (5, 35 mg, 91.05 μmol, 19% yield) as an off-white solid.

LCMS (ES+): m/z 384.2 [M+H]⁺

### 3-[[4-[1-(2,6-dioxo-3-piperidyl)-3-methyl-2-oxo-benzimidazol-5-yl]-1-piperidyl]methyl]cyclobutanecarboxylic acid (6)

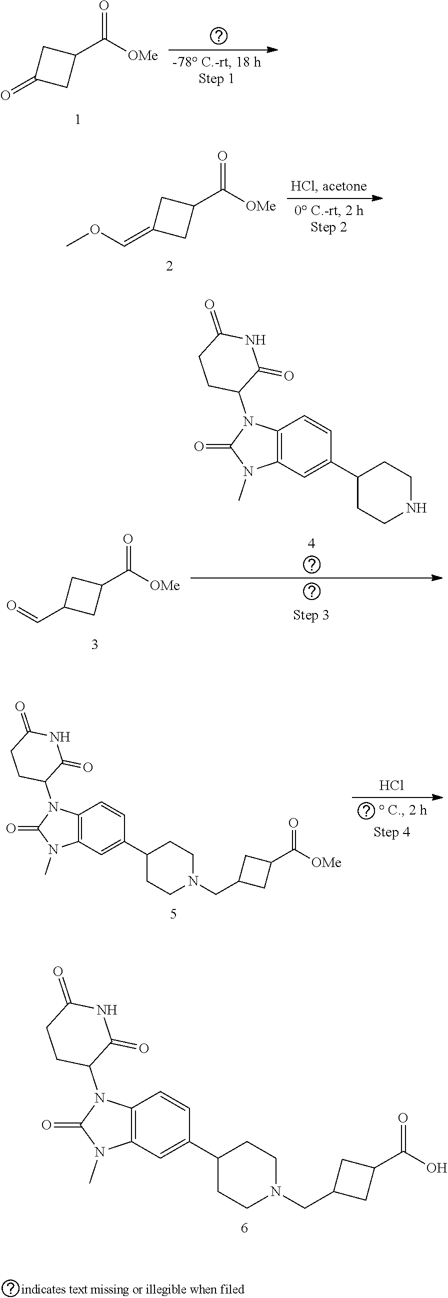

### Step 1: methyl 3-(methoxymethylene)cyclobutanecarboxylate (2)

Into a 250 mL two neck round bottom flask containing a well-stirred suspension of (methoxymethyl)triphenylphosphonium bromide (19.95 g, 51.51 mmol) in THF (100 mL) at 0° C. under nitrogen atmosphere was added LHMDS (1.0 M solution in THF) (46.83 mmol, 46.8 mL) and the resulting reaction mixture was stirred 30 min. A solution of methyl 3-oxocyclobutanecarboxylate (1, 3.0 g, 23.41 mmol) in THF (100 mL). After 18 h, the reaction mixture was slowly quenched with water (20 mL) at 0° C. and extracted with EtOAc (2×50 mL). The combined organic layers were dried over Na₂SO₄ filtered and purified by flash silica gel column chromatography to afford methyl 3-(methoxymethylene)cyclobutanecarboxylate (2, 0.61 g, 3.47 mmol, 15% yield) as an off-white solid.

### Step 2: Methyl 3-formylcyclobutanecarboxylate (3)

Into a 250 mL single neck round bottom flask containing a well-stirred solution of methyl 3-(methoxymethylene)cyclobutanecarboxylate (2, 610 mg, 3.28 mmol) in acetone (5 mL) at 0° C. was added 1N HCl (2.74 mmol, 2.74 mL) dropwise. The reaction mixture was stirred at room temperature for 2 h. The reaction mixture was neutralized with NaHCO₃ solution (10 mL) and extracted with Ethyl acetate (3×50 mL). The combined organic layers were dried over sodium sulfate and concentrated under reduced pressure to obtain methyl 3-formylcyclobutanecarboxylate (3, 420 mg, 2.54 mmol, 77% yield).

### Step 3: Methyl 3-[[4-[1-(2,6-dioxo-3-piperidyl)-3-methyl-2-oxo-benzimidazol-5-yl]-1-piperidyl]methyl]cyclobutanecarboxylate (5)

Into a 50 mL single neck round bottom flask containing a well-stirred solution of methyl 3-formylcyclobutanecarboxylate (3, 479.05 mg, 2.89 mmol) and 3-[3-methyl-2-oxo-5-(4-piperidyl)benzimidazol-1-yl]piperidine-2,6-dione (4, 0.9 g, 2.63 mmol) in anhydrous DMSO 3b/(0.3 mL) were added anhydrous sodium acetate (1.08 g, 13.14 mmol, 704.68 μL) and MP-Cyanoborohydride (1.05 g, 2.10 mmol). After 30 min, acetic acid (1.89 g, 31.54 mmol, 1.80 mL) was added and stirring was continued for another 16 h. The solvent was evaporated to afford the crude material that was purified by reverse-phase column chromatography [Column: Redisep C18-300 g; Mobile Phase A: 0.1% Formic acid in water and Mobile Phase B: MeCN] to obtain methyl 3-[[4-[1-(2,6-dioxo-3-piperidyl)-3-methyl-2-oxo-benzimidazol-5-yl]-1-piperidyl]methyl]cyclobutanecarboxylate (5, 620 mg, 1.14 mmol, 43% yield, formic acid salt) as a white solid.

LCMS (ES+): m/z 469.2 [M+H]⁺

### Step 4: 3-[[4-[1-(2,6-dioxo-3-piperidyl)-3-methyl-2-oxo-benzimidazol-5-yl]-1-piperidyl]methyl]cyclobutanecarboxylic acid (6)

Into a 50 mL sealed-tube containing a well-stirred solution of methyl 3-[[4-[1-(2,6-dioxo-3-piperidyl)-3-methyl-2-oxo-benzimidazol-5-yl]-1-piperidyl]methyl]cyclobutanecarboxylate (5, 300 mg, 601.86 μmol) in THF (5 mL) was added HCl (1.5 M, 4.5 mmol, 3 mL) and the resulting mixture was heated at 65° C. for 2 h. The reaction mixture was concentrated under reduced pressure to obtain 3-[[4-[1-(2,6-dioxo-3-piperidyl)-3-methyl-2-oxo-benzimidazol-5-yl]-1-piperidyl]methyl]cyclobutanecarboxylic acid (6, 280 mg, 531.62 μmol, 88% yield, HCl salt)

LCMS (ES+): m/z 455.6 [M+H]⁺

### 2-[1-[1-(2,6-dioxo-3-piperidyl)-3-methyl-2-oxo-benzimidazol-5-yl]-3,5-dimethyl-pyrazol-4-yl]acetic acid (8)

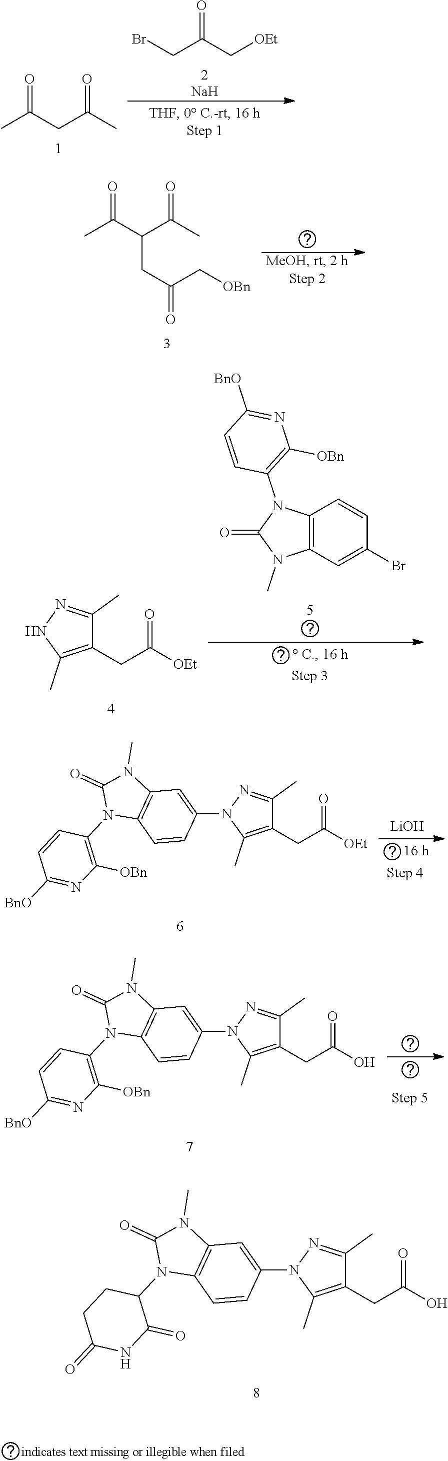

### Step 1: ethyl 3-acetyl-4-oxo-pentanoate (3)

Into a 250 mL two neck round bottom flask containing a well-stirred suspension of sodium hydride (60% dispersion in mineral oil) (918.53 mg, 23.97 mmol) in anhydrous THF (30 mL) at 0° C. was added a solution of pentane-2,4-dione (1, 2.0 g, 19.98 mmol, 2.06 mL) in THF (30 mL) and the resulting mixture was stirred for 1 h at room temperature. To this, ethyl 2-bromoacetate (4.00 g, 23.97 mmol, 2.65 mL) in THF (30 mL) was added at 0° C. and stirring was continued at room temperature for 15 h. The reaction was quenched with saturated ammonium chloride solution (30 mL) and extracted with EtOAc (3×200 mL). The combined layers were dried over sodium sulfate and concentrated under reduced pressure to afford ethyl 3-acetyl-4-oxo-pentanoate (3, 5 g, 8.14 mmol, 41% yield, 30% purity) as yellow oil.

LCMS (ES+): m/z 187.2 [M+H]⁺

### Step 2: ethyl 2-(3,5-dimethyl-1H-pyrazol-4-yl)acetate (4)

Into a 250 mL single neck round bottom flask containing a well-stirred solution of ethyl 3-acetyl-4-oxo-pentanoate (3, 5.0 g, 8.06 mmol) in anhydrous methanol (5 mL) was added hydrazine monohydrate (1.61 g, 32.22 mmol, 1.57 mL). After 2 h, the solvent was evaporated under reduced pressure to afford the crude material that was purified by preparative HPLC [Column: XSelect C18 (250×19) mm, 5 microns; Mobile phase A: 10 mM ammonium acetate in water and Mobile Phase B: MeCN] to afford ethyl 2-(3,5-dimethyl-1H-pyrazol-4-yl)acetate (4, 600 mg, 3.24 mmol, 40% yield) as colorless oil.

UPLC-MS (ES+): m/z 183.2 [M+H]⁺

### Step 3: ethyl 2-[1-[1-(2,6-dibenzyloxy-3-pyridyl)-3-methyl-2-oxo-benzimidazol-5-yl]-3,5-dimethyl-pyrazol-4-yl]acetate (6)

Into a 100 mL sealed tube containing a well-stirred solution of ethyl 2-(3,5-dimethyl-1H-pyrazol-4-yl)acetate (4, 533.63 mg, 2.87 mmol) and 5-bromo-1-(2,6-dibenzyloxy-3-pyridyl)-3-methyl-benzimidazol-2-one (5, 1.3 g, 2.39 mmol) in anhydrous DMF (20 mL) were added trans-N,N′-dimethylcyclohexane-1,2-diamine (408.22 mg, 2.87 mmol), copper (I) iodide (455.49 mg, 2.39 mmol) and anhydrous potassium carbonate (826.37 mg, 5.98 mmol). The mixture was heated at 130° C. for 16 h. The reaction mixture was passed through Celite and washed with EtOAc (50 mL). The filtrate was concentrated under reduced pressure and purified by flash column chromatography (230-400 silica gel, 55% EtOAc in Pet ether) to afford ethyl 2-[1-[1-(2,6-dibenzyloxy-3-pyridyl)-3-methyl-2-oxo-benzimidazol-5-yl]-3,5-dimethyl-pyrazol-4-yl]acetate (6, 700 mg, 913.40 μmol, 38% yield) as brown gummy solid.

UPLC-MS (ES+): m/z 618.8 [M+H]⁺

### Step 4: 2-[1-[1-(2,6-dibenzyloxy-3-pyridyl)-3-methyl-2-oxo-benzimidazol-5-yl]-3,5-dimethyl-pyrazol-4-yl]acetic acid (7)

Into a 100 mL single neck round bottom flask containing a stirred solution ethyl 2-[1-[1-(2,6-dibenzyloxy-3-pyridyl)-3-methyl-2-oxo-benzimidazol-5-yl]-3,5-dimethyl-pyrazol-4-yl]acetate (650 mg, 841.84 μmol) in water (1.98 mL) and THF (19.83 mL) was added lithium hydroxide monohydrate (353.24 mg, 8.42 mmol). After 16 h, the reaction mixture was concentrated, diluted with water (10 mL) and acidified with 1.5N HCl. The precipitate was filtered and dried to afford 2-[1-[1-(2,6-dibenzyloxy-3-pyridyl)-3-methyl-2-oxo-benzimidazol-5-yl]-3,5-dimethyl-pyrazol-4-yl]acetic acid (7, 400 mg, 587.45 μmol, 70% yield, HCl salt) as an off-white solid.

LCMS (ES+): m/z 590.7 [M+H]f

### Step 5: 2-[1-[1-(2,6-dioxo-3-piperidyl)-3-methyl-2-oxo-benzimidazol-5-yl]-3,5-dimethyl-pyrazol-4-yl]acetic acid (8)

Into a 100 mL single neck round bottom flask containing a well-stirred solution of 2-[1-[1-(2,6-dibenzyloxy-3-pyridyl)-3-methyl-2-oxo-benzimidazol-5-yl]-3,5-dimethyl-pyrazol-4-yl]acetic acid (7, 400 mg, 624.11 μmol) in anhydrous DMF (2 mL) and 1,4-dioxane (10 mL) was added palladium hydroxide on carbon (20 wt. % 50% water) (800 mg, 5.70 mmol). The contents were stirred for 16 h under hydrogen gas bladder. The reaction mixture was passed through Celite and washed with dioxane (100 mL). The filtrate was concentrated under reduced pressure and washed with MTBE (10 mL) to afford 2-[1-[1-(2,6-dioxo-3-piperidyl)-3-methyl-2-oxo-benzimidazol-5-yl]-3,5-dimethyl-pyrazol-4-yl]acetic acid (8, 250 mg, 605.36 μmol, 97% yield) as white solid.

LCMS (ES+): m/z 412.2 [M+H]⁺

### 2-[4-[1-(2,6-dioxo-3-piperidyl)-3-methyl-2-oxo-benzimidazol-5-yl]cyclohex-3-en-1-yl]acetic acid (2)

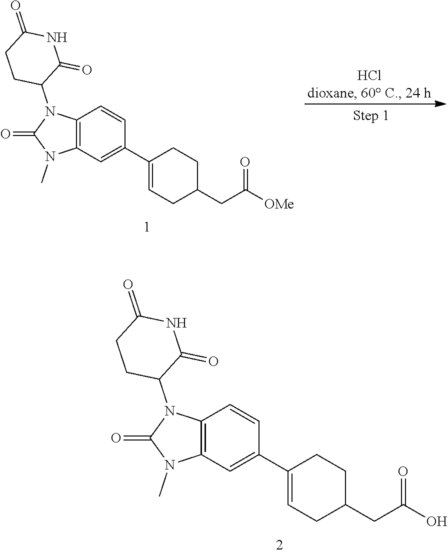 [SMILES: COC(=O)CC1CC=C(c2ccc3c(c2)n(C)c(=O)n3C2CCC(=O)NC2=O)CC1 ; "HCldioxane, 60° C., 24 h" ; Cn1c(=O)n(C2CCC(=O)NC2=O)c2ccc(C3=CCC(CC(=O)O)CC3)cc21]

### Step 2-[4-[1-(2,6-dioxo-3-piperidyl)-3-methyl-2-oxo-benzimidazol-5-yl]cyclohex-3-en-1-yl]acetic acid (2)

Into a 250 mL sealed tube containing a well-stirred solution of methyl 2-[4-[1-(2,6-dioxo-3-piperidyl)-3-methyl-2-oxo-benzimidazol-5-yl]cyclohex-3-en-1-yl]acetate (1, 1 g, 802.04 μmol) in 1,4-dioxane (25 mL) was added hydrochloride solution (1.5 M, 18.58 mL) under nitrogen atmosphere. The reaction was stirred at 60° C. for 24 h. The reaction mixture was concentrated under reduced pressure and purified by reverse phase column chromatography [Column: Redisep C18, 120 g C18; Mobile phase A: 0.1% TFA in water and Mobile phase B: ACN] to afford 2-[4-[1-(2,6-dioxo-3-piperidyl)-3-methyl-2-oxo-benzimidazol-5-yl]cyclohex-3-en-1-yl]acetic acid (2, 60 mg, 150.35 μmol, 19% yield) as a brown solid.

LCMS (ES+): m/z 398.2 [M+H]⁺

### 2-[4-[1-(2,6-dioxo-3-piperidyl)-2-oxo-benzo[cd]indol-6-yl]phenyl]acetic acid (7)

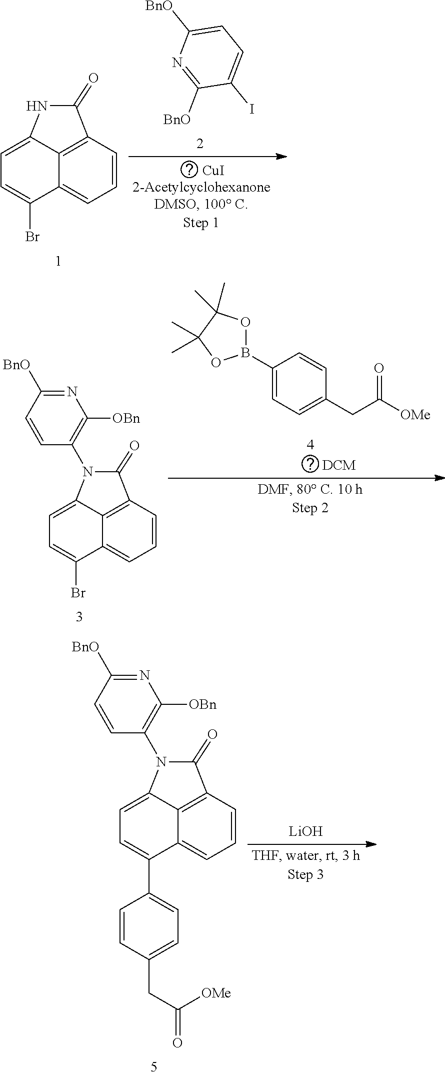

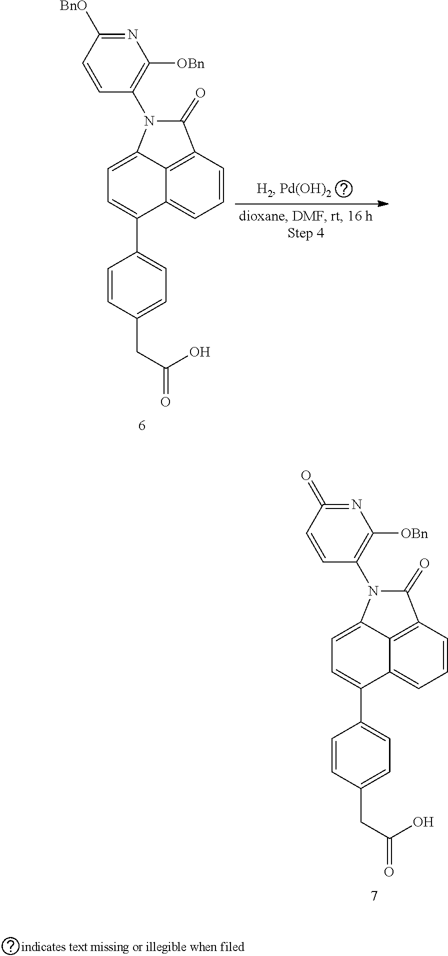

### Step 1: 6-bromo-1-(2,6-dibenzyloxy-3-pyridyl)benzo[cd]indol-2-one (3)

To a stirred solution of 6-bromo-1H-benzo[cd]indol-2-one (1, 2 g, 8.06 mmol) and 2,6-dibenzyloxy-3-iodo-pyridine (2, 4.37 g, 10.48 mmol) in DMSO (10 mL) in a sealed tube was added Ceasium carbonate (7.88 g, 24.19 mmol) and degassed for 10 min. Subsequently, CuI (307.08 mg, 1.61 mmol, 54.64 μL) was added to it followed by 2-Acetylcyclohexanone (226.03 mg, 1.61 mmol, 213.23 μL) and degassed at argon atmosphere for 10 min. The reaction mixture was heated at 100° C. for 18 h. The reaction mixture was then allowed to come to RT, filtered and was extracted with ethyl acetate. The organic phase was washed with brine, and finally dried over anhyd. Na₂SO₄. The solvent was evaporated and the residue was purified by column chromatography on silica gel to furnish the desired product 6-bromo-1-(2,6-dibenzyloxy-3-pyridyl)benzo[cd]indol-2-one (3, 700 mg, 1.17 mmol, 15% yield) as yellow solid.

LCMS (ES+): m/z 539.2 [M+H]+

### Step 2: methyl 2-[4-[1-(2,6-dibenzyloxy-3-pyridyl)-2-oxo-benzo[cd]indol-6-yl]phenyl]acetate (5)

Into a 100 mL pressure tube containing a solution of 6-bromo-1-(2,6-dibenzyloxy-3-pyridyl)benzo[cd]indol-2-one (3, 700 mg, 1.30 mmol) in DMF (15 mL) was added methyl 2-[4-(4,4,5,5-tetramethyl-1,3,2-dioxaborolan-2-yl)phenyl]acetate (4, 431.62 mg, 1.56 mmol) and Cesium fluoride (494.66 mg, 3.26 mmol). The mixture was degassed by bubbling nitrogen gas for 5 min. Then Pd(dppf)Cl₂·DCM complex (106.37 mg, 130.26 μmol) was added and the reaction mixture was heated at 90° C. for 16 h. The reaction mixture was filtered through Celite and washed with ethyl acetate (200 mL). The filtrate was concentrated and the crude compound was purified by column chromatography (50-60% EtOAc in pet ether) to afford methyl 2-[4-[1-(2,6-dibenzyloxy-3-pyridyl)-2-oxo-benzo[cd]indol-6-yl]phenyl]acetate (5, 800 mg, 1.19 mmol, 91.11% yield, 90% purity) as a pale yellow solid.

UPLC-MS (ES+): m/z 607.3 [M+H]⁺

### Step 3: 2-[4-[1-(2,6-dibenzyloxy-3-pyridyl)-2-oxo-benzo[cd]indol-6-yl]phenyl]acetic acid (5)

Into a 50 mL round bottom flask containing a well-stirred solution of methyl 2-[4-[1-(2,6-dibenzyloxy-3-pyridyl)-2-oxo-benzo[cd]indol-6-yl]phenyl]acetate (5, 800 mg, 1.19 mmol) in THF (4 mL) and water (2 mL) was added lithium hydroxide (99.61 mg, 2.37 mmol) at room temperature. The reaction mixture was stirred at room temperature for 3 h. After completion of the reaction, the reaction mixture was concentrated under vacuum to afford the residue which was acidified with 1.5 N HCl. The aqueous layer was extracted twice with ethyl acetate (2×100 mL). The combined organic layers was washed with water followed by brine solution (100 mL) and concentrated under vacuum to afford 2-[4-[1-(2,6-dibenzyloxy-3-pyridyl)-2-oxo-benzo[cd]indol-6-yl]phenyl]acetic acid (6, 750 mg, 1.07 mmol, 90.41% yield, 90% purity, HCl salt) as a brown solid.

UPLC-MS (ES+): m/z 594.2 [M+H]⁺

### Step 4: 2-[4-[1-(2,6-dioxo-3-piperidyl)-2-oxo-benzo[cd]indol-6-yl]phenyl]acetic acid (7)

Into a 100 mL single neck round bottom flask containing a well-stirred solution of 2-[4-[1-(2,6-dibenzyloxy-3-pyridyl)-2-oxo-benzo[cd]indol-6-yl]phenyl]acetic acid (6, 750 mg, 1.14 mmol, 000) in 1,4-dioxane (10 mL) was added 20 wt. % palladium hydroxide on carbon, (799.76 mg, 1.14 mmol, 20% purity) and the suspension was stirred under hydrogen atmosphere at room temperature for 16 h. The reaction mixture was filtered through Celite, washed with 1 4-dioxane (100 mL). The filtrate was concentrated under reduced pressure which was purified by reverse prep HPLC [XSelect C18 (150×19) mm 5 microns; Mobile phase: 0.1% TFA in water; Mobile phase B: MeCN] to obtain 2-[4-[1-(2,6-dioxo-3-piperidyl)-2-oxo-benzo[cd]indol-6-yl]phenyl]acetic acid (7, 440 mg, 1.05 mmol, 92.52% yield, 99.25% purity) as a yellow solid.

LCMS (ES+): m/z 415.2 [M+H]⁺

¹H-NMR (400 MHz, DMSO-d6): δ 12.41 (s, 1H), 11.16 (s, 1H), δ 8.19 (q, J=6.80 Hz, 2H), 7.87 (t, J=7.60 Hz, 1H), 7.54 (q, J=2.40 Hz, 3H), 7.45 (d, J=8.00 Hz, 2H), 7.27 (d, J=7.60 Hz, 1H), 5.52 (q, J=5.20 Hz, 1H), 3.69 (s, 2H), 2.99-2.94 (m, 1H), 2.87-2.79 (m, 1H), 2.70-2.66 (m, 1H), 2.14 (t, J=5.20 Hz, 1H).

### 2-[1-[1-(2,6-dioxo-3-piperidyl)-2-oxo-benzo[cd]indol-6-yl]-4-piperidyl]acetic acid (11)

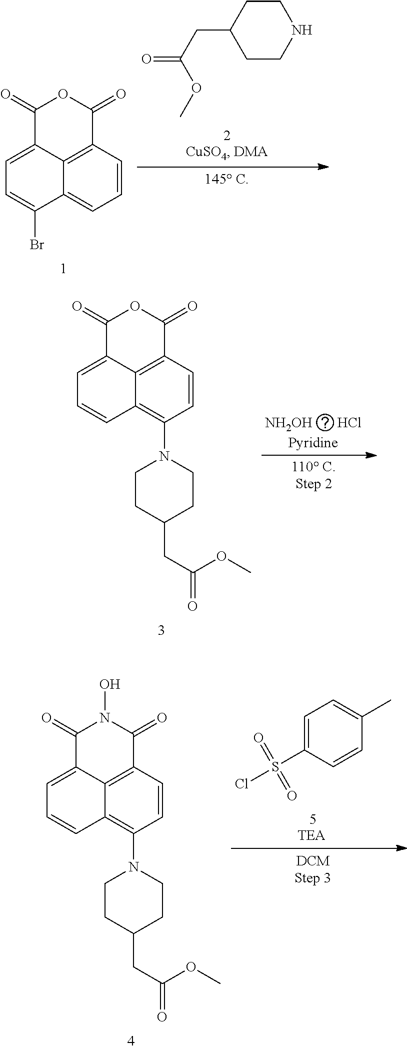

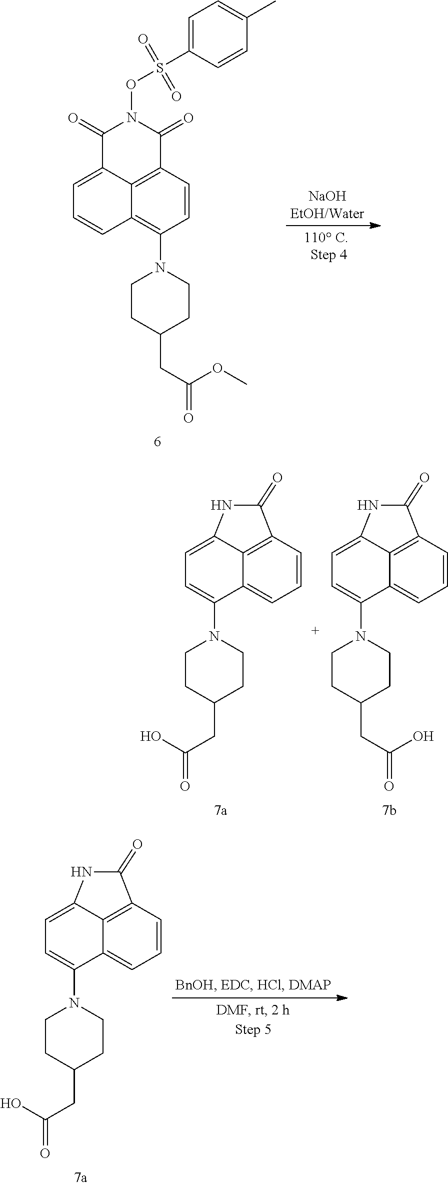

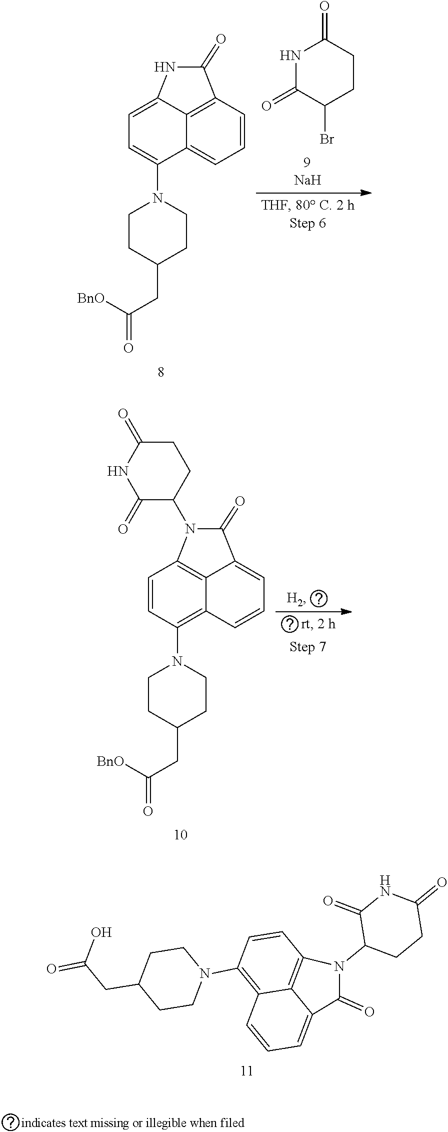

### Step 1: methyl 2-(1-(1,3-dioxo-1H,3H-benzo[de]isochromen-6-yl)piperidin-4-yl)acetate (3)

Into a 250 mL sealed tube containing a well-stirred solution of 6-bromo-1H,3H-benzo[de]isochromene-1,3-dione (1, 5 g, 18.05 mmol) and methyl 2-(4-piperidyl)acetate (2, 7.09 g, 45.11 mmol) in DMA (50 mL) was added Copper(II) sulfate pentahydrate (1.35 g, 5.41 mmol). The resultant reaction mixture was stirred for 5 h at 145° C. Cold water (100 mL) was added and the precipitate was filtered, washed with water (30 mL) and purified by column chromatography eluting with ethyl acetate/pet-ether to afford methyl 2-(1-(1,3-dioxo-1H,3H-benzo[de]isochromen-6-yl)piperidin-4-yl)acetate (3, 10.2 g, 9.33 mmol, 52% yield, 32% purity) as a yellow solid.

LCMS (ES+): m/z 354.2 [M+H]⁺.

### Step 2: methyl 2-[1-(2-hydroxy-1,3-dioxo-benzo[de]isoquinolin-6-yl)-4-piperidyl]acetate (4)

Into a 250 mL round bottom flask containing a well-stirred solution of methyl 2-(1-(1,3-dioxo-1H,3H-benzo[de]isochromen-6-yl)piperidin-4-yl)acetate (3, 10.2 g, 9.24 mmol) in Pyridine (40 mL) was added Hydroxylamine hydrochloride (641.87 mg, 9.24 mmol, 384.36 μL) The reaction mixture was stirred at 110° C. for 2 h. The volatiles were evaporated under reduced pressure to afford methyl 2-[1-(2-hydroxy-1,3-dioxo-benzo[de]isoquinolin-6-yl)-4-piperidyl]acetate (4, 4.8 g, 9.06 mmol, 98% yield) as a yellow gummy liquid. The material was used in the next step without purification.

LCMS (ES+): m/z 369.2 [M+H]⁺.

### Step 3: methyl 2-[1-[1,3-dioxo-2-(p-tolylsulfonyloxy)benzo[de]isoquinolin-6-yl]-4-piperidyl]acetate (6)

In to a 250 mL round bottom flask containing a well-stirred solution of methyl 2-[1-(2-hydroxy-1,3-dioxo-benzo[de]isoquinolin-6-yl)-4-piperidyl]acetate (4, 17.1 g, 32.29 mmol) in DCM (50 mL) were added Triethylamine (4.90 g, 48.43 mmol, 6.75 mL) and 4-methylbenzenesulfonyl chloride (5, 7.39 g, 38.75 mmol). After 10 h the reaction was quenched with water (60 mL) and extracted with DCM (3×50 mL). The organic layer was dried over sodium sulfate, filtered, concentrated and purified by silicagel chromatography (100-200 mesh) using 45-50% EtOAc/pet ether as a gradient to afford methyl 2-[1-[1,3-dioxo-2-(p-tolylsulfonyloxy)benzo[de]isoquinolin-6-yl]-4-piperidyl]acetate (6, 11.97 g, 16.96 mmol, 53% yield) as a yellow solid.

LCMS: m/z 523.2 [M+H]⁺

### Step 4: 2-[1-(2-oxo-1H-benzo[cd]indol-6-yl)-4-piperidyl]acetic acid (7a) and 2-[1-(2-oxo-1H-benzo[cd]indol-5-yl)-4-piperidyl]acetic acid (7b)

Into a 250 mL three neck round bottom flask containing a suspension of methyl 2-[1-[1,3-dioxo-2-(p-tolylsulfonyloxy)benzo[de]isoquinolin-6-yl]-4-piperidyl]acetate (6, 11.97 g, 16.95 mmol) in Ethanol (20 mL) and Water (10 mL) was added 10% NaOH solution (23.05 g, 576.32 mmol, 50 mL) and the resulting mixture was heated at reflux for 4 h. The mixture was carefully acidified by adding concentrated HCl dropwise to precipitate a solid that was filtered, washed with cold water and dried. The material was purified by reverse phase prep HPLC [Purification method: Column: X-Select, C18 (150×10) mm, 5 micron; Mobile phase A: 0.1% FA in water and Mobile phase B: MeCN] to afford 2-[1-(2-oxo-1H-benzo[cd]indol-6-yl)-4-piperidyl]acetic acid (7a, 1.7 g, 3.62 mmol, 21% yield, Formic acid salt) as a yellow solid. LCMS: m/z 311.2 [M+H]+ and 2-[1-(2-oxo-1H-benzo[cd]indol-5-yl)-4-piperidyl]acetic acid (7b, 0.98 g, 1.70 mmol, 10% yield, Formic acid salt) as a yellow solid. LCMS: m/z 311.2 [M+H]+.

### Step 5: benzyl 2-[1-(2-oxo-1H-benzo[cd]indol-6-yl)-4-piperidyl]acetate (8)

Into a 50 mL round bottom flask containing a well-stirred solution of 2-[1-(2-oxo-1H-benzo[cd]indol-6-yl)-4-piperidyl]acetic acid (7a, 500 mg, 902.22 μmol) in DMF (2 mL) was added EDC (77.83 mg, 406.00 μmol). After 5 min, phenylmethanol (97.56 mg, 902.22 mol, 92.92 μL) and DMAP (330.67 mg, 2.71 mmol) were added and stirring was continued at room temperature for 2 h. The mixture was poured into water and extracted with EtOAc (2×20 mL). The combined organic layers were dried over Na₂SO₄ and concentrated under reduced pressure and purified by flash column chromatography (230-400 mesh silica gel, 65% EtOAc in pet ether) to obtain benzyl 2-[1-(2-oxo-1H-benzo[cd]indol-6-yl)-4-piperidyl]acetate (8, 350 mg, 374.94 μmol, 42% yield, 43% purity) as a brown solid.

LCMS (ES+): m/z 401.2 [M+H]⁺

### Step 6: benzyl 2-[1-[1-(2,6-dioxo-3-piperidyl)-2-oxo-benzo[cd]indol-6-yl]-4-piperidyl]acetate (10)

To a suspension of benzyl 2-[1-(2-oxo-1H-benzo[cd]indol-6-yl)-4-piperidyl]acetate (8, 600 mg, 1.17 mmol) in tetrahydrofuran (10 mL) was added sodium hydride (60% dispersion in oil) (467.41 mg, 11.69 mmol) at 0° C. The reaction mixture was stirred at 25° C. for 30 min and then cooled to 0° C. 3-bromo piperidine-2,6-dione (9, 897.56 mg, 4.67 mmol) in tetrahydrofuran (10 mL) was added and the resultant mixture was heated at 60° C. for 2 h. The reaction was quenched with ammonium chloride solution and extracted with EtOAc (2×50 mL). The combined organic layers were washed with brine solution (30 mL), dried over Na₂SO₄ and then concentrated to afford benzyl 2-[1-[1-(2,6-dioxo-3-piperidyl)-2-oxo-benzo[cd]indol-6-yl]-4-piperidyl]acetate (10, 500 mg, 878.87 μmol, 75% yield) which was used in the next step directly.

LCMS (ES+): m/z 512.2 [M+H]⁺

### Step 7: 2-[1-[1-(2,6-dioxo-3-piperidyl)-2-oxo-benzo[cd]indol-6-yl]-4-piperidyl]acetic acid (11)

To a stirred solution of benzyl 2-[1-[1-(2,6-dioxo-3-piperidyl)-2-oxo-benzo[cd]indol-6-yl]-4-piperidyl]acetate (10, 600 mg, 867.92 μmol) in 1,4-dioxane (50 mL) was added palladium hydroxide on carbon (20 wt. %, 50% water) (1.1 g, 1.57 mmol) and the resultant mixture was stirred under hydrogen atmosphere for 2 h at 25° C. The reaction mixture was filtered through Celite and washed with 1,4-dioxane (100 mL). The filtrate was concentrated under reduced pressure (45° C.) to afford 2-[1-[1-(2,6-dioxo-3-piperidyl)-2-oxo-benzo[cd]indol-6-yl]-4-piperidyl]acetic acid (11, 398 mg, 635.22 μmol, 73% yield) which was used in the next step without further purification.

LCMS (ES+): m/z 422.2 [M+H]⁺

### 2-[4-[1-(2,6-dioxo-3-piperidyl) indolin-5-yl]-1-piperidyl]acetic acid (10)

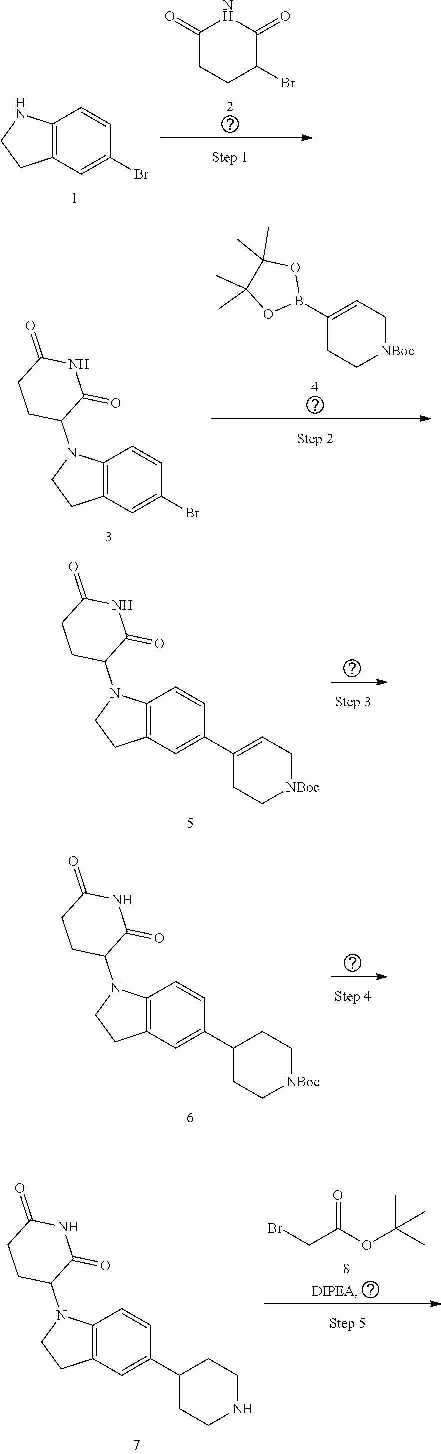

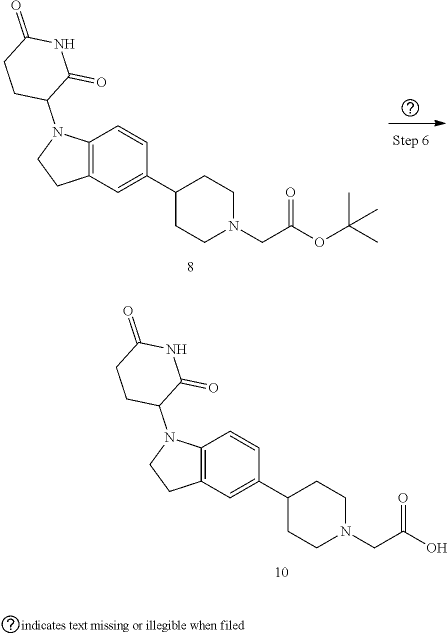 [SMILES: CC(C)(C)OC(=O)CN1CCC(c2ccc3c(c2)CCN3C2CCC(=O)NC2=O)CC1 ; O=C(O)CN1CCC(c2ccc3c(c2)CCN3C2CCC(=O)NC2=O)CC1]

### Step 1: 3-(5-bromoindolin-1-yl)piperidine-2,6-dione (3)

A well-stirred solution of 5-bromoindoline (1, 1.0 g, 5.05 mmol) in anhydrous THF (100 mL) was treated with Sodium hydride (60% dispersion in mineral oil) (1.93 g, 50.49 mmol, 60% purity) at 0° C. under inert atmosphere. The reaction mixture was stirred for 1 h at rt. 3-bromopiperidine-2,6-dione (2, 3.03 g, 15.15 mmol) in THF (8 mL) was added to the reaction mixture and stirred for 16 h at 60° C. The reaction was quenched with NH4Cl solution (15 mL) at 0° C. and extracted with Ethyl Acetate (2×150 mL). Combined organic layer was concentrated and purified by column chromatography (230-400 silica gel) with 50-60% Ethyl Acetate in Pet ether to afford 3-(5-bromoindolin-1-yl)piperidine-2,6-dione (3, 550 mg, 1.60 mmol, 32% yield) as a pale yellow solid.

LCMS (ES+): m/z 311.0 [M+H]+

### Step 2: tert-butyl 4-[1-(2,6-dioxo-3-piperidyl)indolin-5-yl]-3,6-dihydro-2H-pyridine-1-carboxylate (5)

Into a 25 mL pressure tube containing a well-stirred solution of 3-(5-bromoindolin-1-yl) piperidine-2,6-dione (3, 300 mg, 834.52 μmol) and tert-butyl 4-(4,4,5,5-tetramethyl-1,3,2-dioxaborolan-2-yl)-3,6-dihydro-2H-pyridine-1-carboxylate (4, 516.08 mg, 1.67 mmol) in DMF (5 mL) was added cesium fluoride (316.92 mg, 2.09 mmol) and Pd(dppf)Cl₂·CH₂Cl₂ (204.45 mg, 250.36 μmol). The reaction mixture was degassed by bubbling nitrogen gas for 10 min. The mixture was then stirred at 90° C. for 16 h. The reaction mixture was filtered through Celite and washed thoroughly with ethyl acetate (150 mL) and the filtrate was washed with water (100 mL) followed by brine (100 mL). The organic layer was dried over Na₂SO₄, filtered and concentrated and purified by flash silica gel column chromatography (70% EtOAc in pet ether) as eluent to afford tert-butyl 4-[1-(2,6-dioxo-3-piperidyl) indolin-5-yl]-3,6-dihydro-2H-pyridine-1-carboxylate (5, 130 mg, 301.77 μmol, 36% yield) as a brown solid.

LCMS (ES+): m/z 412.3 [M+H]⁺

### Step 3: tert-butyl 4-[1-(2,6-dioxo-3-piperidyl) indolin-5-yl] piperidine-1-carboxylate (6)

Into a 50 mL single neck round bottom flask containing a well-stirred solution of tert-butyl 4-[1-(2,6-dioxo-3-piperidyl)indolin-5-yl]-3,6-dihydro-2H-pyridine-1-carboxylate (5, 130 mg, 302.40 mol) in 1,4-dioxane (1.5 mL) was added 20 wt. % Palladium hydroxide on carbon (106.17 mg, 151.20 μmol, 20% purity). The suspension was stirred at room temperature for 16 h under hydrogen atmosphere. The reaction mixture was filtered through Celite, washed with 1,4-dioxane (150 mL) and concentrated under reduced pressure to afford tert-butyl 4-[1-(2,6-dioxo-3-piperidyl) indolin-5-yl] piperidine-1-carboxylate (6, 120 mg, 275.69 μmol, 91% yield) as a brown solid.

LCMS (ES+): m/z 358.2 [M−tbu+H]⁺

### Step 4: 3-[5-(4-piperidyl) indolin-1-yl] piperidine-2,6-dione (7)

Into a 50 mL single neck round bottom flask containing a well-stirred solution of tert-butyl 4-[1-(2,6-dioxo-3-piperidyl)indolin-5-yl]piperidine-1-carboxylate (6, 120 mg, 275.69 μmol) in DCM (2 mL) was added TFA (628.70 mg, 5.51 mmol, 424.80 μL) and the reaction mixture was stirred at room temperature for 2 h. After completion of the reaction, the reaction mixture was concentrated to dryness and washed with MTBE (25 mL) to afford 3-[5-(4-piperidyl)indolin-1-yl]piperidine-2,6-dione (7, 120 mg, 255.69 μmol, 93% yield, TFA salt) as a brown solid.

LCMS (ES+): m/z 314.2 [M+H]⁺

### Step 5: tert-butyl 2-[4-[1-(2,6-dioxo-3-piperidyl) indolin-5-yl]-1-piperidyl]acetate (9)

Into a 20 mL vial containing a well-stirred solution of 3-[5-(4-piperidyl) indolin-1-yl]piperidine-2,6-dione (7, 120 mg, 255.49 μmol, TFA salt) in DMF (1 mL) was added DIPEA (165.10 mg, 1.28 mmol, 222.51 μL) and tert-butyl bromoacetate (8, 39.87 mg, 204.39 μmol, 29.98 μL) at 0° C. After 30 min, the reaction was quenched with cold water at 0° C. and extracted with ethyl acetate (2×100 mL). The combined organic layer was dried over anhydrous Na₂SO₄, filtered and concentrated under reduced pressure to afford tert-butyl 2-[4-[1-(2,6-dioxo-3-piperidyl) indolin-5-yl]-1-piperidyl]acetate (9, 90 mg, 178.93 μmol, 70% yield) as a brown solid.

LCMS (ES+): m/z 428.2 [M+H]⁺

### Step 6: 2-[4-[1-(2,6-dioxo-3-piperidyl) indolin-5-yl]-1-piperidyl]acetic acid (10)

Into a 25 mL single neck round bottom flask containing a well-stirred solution of tert-butyl 2-[4-[1-(2,6-dioxo-3-piperidyl)indolin-5-yl]-1-piperidyl]acetate (9, 90 mg, 178.93 μmol) in DCM (1.5 mL) was added TFA (408.05 mg, 3.58 mmol, 275.71 μL) and the reaction mixture was stirred at ambient temperature for 3 h. The reaction mixture was concentrated to dryness and washed with MTBE (50 mL) and purified by reverse phase prep HPLC [Purification method: Column: XSelect C18 (150×19) mm 5 micron; Mobile phase A: 0.1% TFA in water; Mobile phase B: MeCN] to afford 2-[4-[1-(2,6-dioxo-3-piperidyl) indolin-5-yl]-1-piperidyl]acetic acid (10, 90 mg, 174.95 μmol, 98% yield, TFA salt) as a brown sticky solid.

LCMS (ES+): m/z 372.2[M+H]⁺

¹HNMR (400 MHz, DMSO-d6): δ 10.81 (s, 1H), 6.91 (s, 1H), 6.82 (d, J=8.00 Hz, 1H), 6.41 (d, J=8.40 Hz, 1H), 4.60-4.57 (m, 1H), 3.40-3.26 (m, 6H), 3.17 (s, 2H), 2.94-2.79 (m, 2H), 2.79-2.69 (m, 1H), 2.21-2.17 (m, 1H), 1.93-1.82 (m, 1H), 1.81-1.71 (m, 4H).

### Preparation of Exemplary Formula (I) Compounds

### 2-[4-[3-(2,6-dioxo-3-piperidyl)-1-methyl-indazol-6-yl]-3,5-dimethyl-pyrazol-1-yl]-N-[5-fluoro-7-hydroxy-6-(1,1,4-trioxo-1,2,5-thiadiazolidin-2-yl)-2-naphthyl]acetamide (Degrader 1)

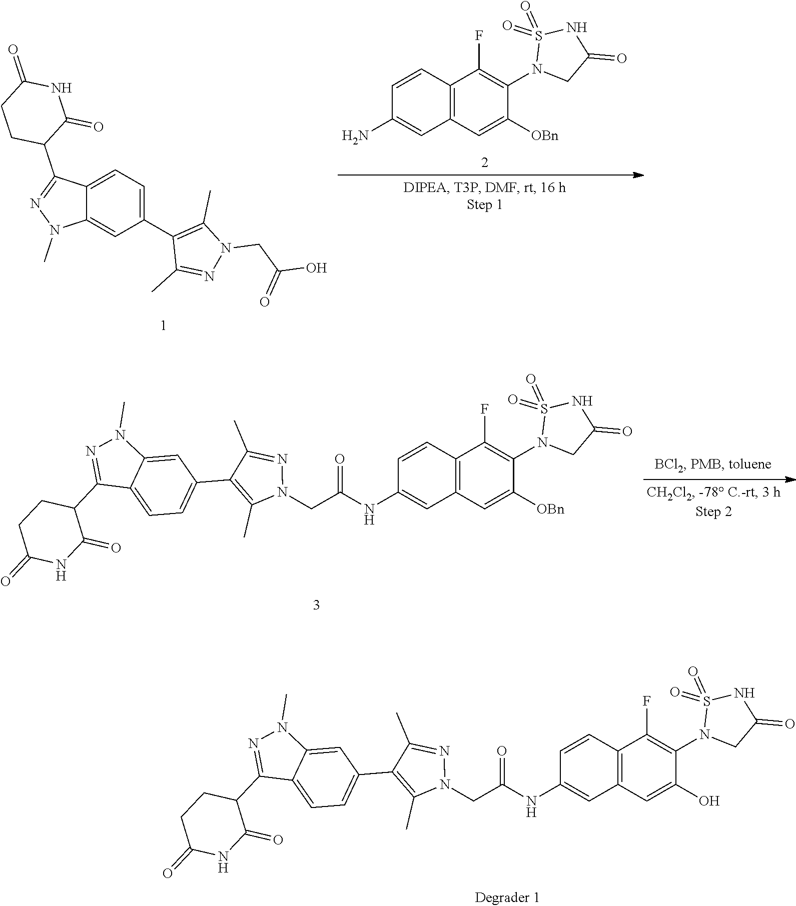 [SMILES: Cc1nn(CC(=O)O)c(C)c1-c1ccc2c(C3CCC(=O)NC3=O)nn(C)c2c1 ; Nc1ccc2c(F)c(N3CC(=O)NS3(=O)=O)c(OCc3ccccc3)cc2c1 ; Cc1nn(CC(=O)Nc2ccc3c(F)c(N4CC(=O)NS4(=O)=O)c(OCc4ccccc4)cc3c2)c(C)c1-c1ccc2c(C3CCC(=O)NC3=O)nn(C)c2c1 ; Cc1nn(CC(=O)Nc2ccc3c(F)c(N4CC(=O)NS4(=O)=O)c(O)cc3c2)c(C)c1-c1ccc2c(C3CCC(=O)NC3=O)nn(C)c2c1]

### Step 1: N-(7-(benzyloxy)-6-(1,1-dioxido-4-oxo-1,2,5-thiadiazolidin-2-yl)-5-fluoronaphthalen-2-yl)-2-(4-(3-(2,6-dioxopiperidin-3-yl)-1-methyl-1H-indazol-6-yl)-3,5-dimethyl-1H-pyrazol-1-yl)acetamide (3)

Into a 8 mL screw-capped vial containing a well-stirred solution of 2-[4-[3-(2,6-dioxo-3-piperidyl)-1-methyl-indazol-6-yl]-3,5-dimethyl-pyrazol-1-yl]acetic acid (1, 123.86 mg, 291.31 μmol) and 5-(6-amino-3-benzyloxy-1-fluoro-2-naphthyl)-1,1-dioxo-1,2,5-thiadiazolidin-3-one (2,150 mg, 264.83 μmol, TFA salt) in DMF (3.0 mL) was added DIPEA (171.13 mg, 1.32 mmol, 230.63 μL) followed by 1-propanephosphonic anhydride (50 Wt % in EtOAc, 252.79 mg, 397.24 μmol, 50% purity) and the resulting mixture was stirred at room temperature for 16 h. The mixture was concentrated under vacuum and acidified with 1.5 N HCl (0.5 mL). The precipitated solid was filtered and dried under vacuum to afford N-(7-(benzyloxy)-6-(1,1-dioxido-4-oxo-1,2,5-thiadiazolidin-2-yl)-5-fluoronaphthalen-2-yl)-2-(4-(3-(2,6-dioxopiperidin-3-yl)-1-methyl-1H-indazol-6-yl)-3,5-dimethyl-1H-pyrazol-1-yl)acetamide (3, 160 mg, 128.35 μmol, 48% yield, HCl salt) as a brown solid.

LCMS (ES+): m/z 779.0 [M+H]⁺

### Step 2: 2-[4-[3-(2,6-dioxo-3-piperidyl)-1-methyl-indazol-6-yl]-3,5-dimethyl-pyrazol-1-yl]-N-[5-fluoro-7-hydroxy-6-(1,1,4-trioxo-1,2,5-thiadiazolidin-2-yl)-2-naphthyl]acetamide (Degrader 1)

Into a 50 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[4-[3-(2,6-dioxo-3-piperidyl)-1-methyl-indazol-6-yl]-3,5-dimethyl-pyrazol-1-yl]acetamide (3, 160 mg, 127.57 μmol, HCl salt) and pentamethylbenzene (94.56 mg, 637.83 μmol, 103.12 μL) in a mixture of toluene (2.5 mL) and DCM (2.5 mL) was added BCl₃ (1.0 M solution in methylene chloride, 2.55 mmol, 2.55 mL) at −78° C. and the resulting mixture was stirred at room temperature for 5 h. The reaction mixture was cooled to −78° C. and quenched with 5% MeOH in DCM (3 mL). Subsequently, the mixture was concentrated under vacuum and purified by reverse-phase preparative HPLC [Column: X-Bridge C8 (150×19) mm, 5 μm; Mobile phase A: 0.1% Formic acid in water and Mobile Phase B: MeCN] to afford 2-[4-[3-(2,6-dioxo-3-piperidyl)-1-methyl-indazol-6-yl]-3,5-dimethyl-pyrazol-1-yl]-N-[5-fluoro-7-hydroxy-6-(1,1,4-trioxo-1,2,5-thiadiazolidin-2-yl)-2-naphthyl]acetamide (Degrader 1, 10 mg, 12.97 μmol, 10% yield, formic acid salt) as an off-white solid.

LCMS (ES+): m/z 689.2 [M+H]⁺

¹H-NMR (400 MHz, DMSO-d6): δ 10.92 (s, 1H), 10.62 (s, 1H), 10.22 (brs, 1H), 8.20 (s, 1H), 7.90 (d, J=9.20 Hz, 1H), 7.75 (d, J=8.40 Hz, 1H), 7.47-7.44 (m, 2H), 7.07 (d, J=9.20 Hz, 1H), 6.97 (d, J=8.80 Hz, 1H), 5.04 (s, 2H), 4.40 (dd, J=5.20, 10.00 Hz, 1H), 4.29 (s, 2H), 4.02 (s, 3H), 2.75-2.60 (m, 2H), 2.48-2.38 (m, 1H), 2.32 (s, 3H), 2.36-2.15 (m, 4H).

### 2-[4-[1-(2,6-dioxo-3-piperidyl)-3-methyl-2-oxo-benzimidazol-5-yl]-3-methyl-pyrazol-1-yl]-N-[5-fluoro-7-hydroxy-6-(1,1,4-trioxo-1,2,5-thiadiazolidin-2-yl)-2-naphthyl]acetamide (Degrader 2)

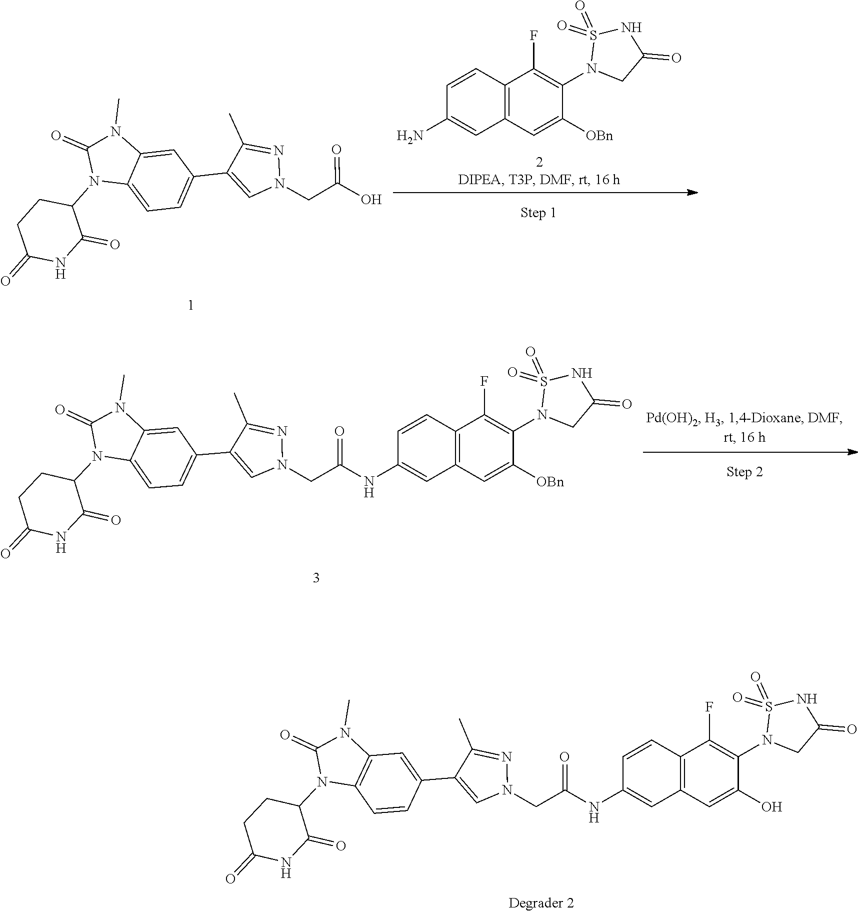 [SMILES: Cc1nn(CC(=O)O)cc1-c1ccc2c(c1)n(C)c(=O)n2C1CCC(=O)NC1=O ; Nc1ccc2c(F)c(N3CC(=O)NS3(=O)=O)c(OCc3ccccc3)cc2c1 ; Cc1nn(CC(=O)Nc2ccc3c(F)c(N4CC(=O)NS4(=O)=O)c(OCc4ccccc4)cc3c2)cc1-c1ccc2c(c1)n(C)c(=O)n2C1CCC(=O)NC1=O ; Cc1nn(CC(=O)Nc2ccc3c(F)c(N4CC(=O)NS4(=O)=O)c(O)cc3c2)cc1-c1ccc2c(c1)n(C)c(=O)n2C1CCC(=O)NC1=O]

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3-methyl-pyrazol-1-yl]acetamide (3)

To a 50 mL single-neck round-bottom flask containing a well-stirred solution of 2-[4-[1-(2,6-dioxo-3-piperidyl)-3-methyl-2-oxo-benzimidazol-5-yl]-3-methyl-pyrazol-1-yl]acetic acid (1, 150 mg, 354.82 μmol) and 5-(6-amino-3-benzyloxy-1-fluoro-2-naphthyl)-1,1-dioxo-1,2,5-thiadiazolidin-3-one (2, 200.97 mg, 354.82 μmol, TFA salt) in anhydrous DMF (3 mL) were added N,N-diisopropylethylamine (275.15 mg, 2.13 mmol, 370.82 μL), followed by 50 wt. % 1-propanephosphonic anhydride solution in ethyl acetate (338.69 mg, 532.23 μmol, 50% purity). After 16 h, the solvent was removed under vacuum and the residue was treated with aq. 1.5 N HCl (5 mL) and the solid precipitated was collected by filtration, co-distilled with toluene (2×5 mL), dried under reduced pressure to obtain N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3-methyl-pyrazol-1-yl]acetamide (3, 190 mg, 148.79 μmol, 42% yield, HCl salt) as a brown solid.

LCMS (ES+): m/z 781 [M+H]⁺

### Step 2: 2-[4-[1-(2,6-dioxo-3-piperidyl)-3-methyl-2-oxo-benzimidazol-5-yl]-3-methyl-pyrazol-1-yl]-N-[5-fluoro-7-hydroxy-6-(1,1,4-trioxo-1,2,5-thiadiazolidin-2-yl)-2-naphthyl]acetamide (Degrader 2)

To a 50 mL single-neck round-bottom flask containing a solution of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3-methyl-pyrazol-1-yl]acetamide (3, 190 mg, 148.79 μmol, HCl salt) in a mixture of 1,4-dioxane (2 mL) and DMF (2 mL) was added 20 wt. % palladium hydroxide on carbon (188.06 mg, 267.83 μmol, 20% purity) and the suspension was stirred under hydrogen atmosphere (bladder) at room temperature. After 16 h, the reaction mixture was filtered and washed with a mixture of 1,4-dioxane (100 mL), DMF (100 mL), followed by a mixture of MeCN (100 mL) and THF (100 mL). The combined filtrate was concentrated to dryness and the crude compound was purified by reverse phase prep HPLC [Purification method: Column: XSelect C18 (250×19) mm 5 micron; Mobile phase A: 0.1% Formic acid in MQ water; Mobile phase B: MeCN], to obtain 2-[4-[1-(2,6-dioxo-3-piperidyl)-3-methyl-2-oxo-benzimidazol-5-yl]-3-methyl-pyrazol-1-yl]-N-[5-fluoro-7-hydroxy-6-(1,1,4-trioxo-1,2,5-thiadiazolidin-2-yl)-2-naphthyl]acetamide (Degrader 2, 48 mg, 57.89 μmol, 39% yield, Formic acid salt) as an off-white solid.

LCMS (ES−): m/z 688.7 [M−H]⁻

¹H-NMR (400 MHz, DMSO-d6): δ 11.11 (s, 1H), 10.52 (brs, 2H), 8.13 (s, 1H), 7.88 (s, 1H), 7.84 (d, J=8.80 Hz, 1H), 7.39 (dd, J=1.60, 9.20 Hz, 1H), 7.17 (d, J=1.20 Hz, 1H), 7.14 (s, 1H), 7.09-7.01 (m, 2H), 6.91 (s, 1H), 5.32 (dd, J=5.20, 12.80 Hz, 1H), 4.95 (s, 2H), 4.40 (s, 2H), 3.30 (s, 3H), 2.90-2.75 (m, 1H), 2.76-2.55 (m, 2H), 2.26 (s, 3H), 2.05-1.92 (m, 1H).

### 2-[4-[1-(2,6-dioxo-3-piperidyl)-3-methyl-2-oxo-benzimidazol-5-yl]-5-methyl-pyrazol-1-yl]-N-[5-fluoro-7-hydroxy-6-(1,1,4-trioxo-1,2,5-thiadiazolidin-2-yl)-2-naphthyl]acetamide (Degrader 3)

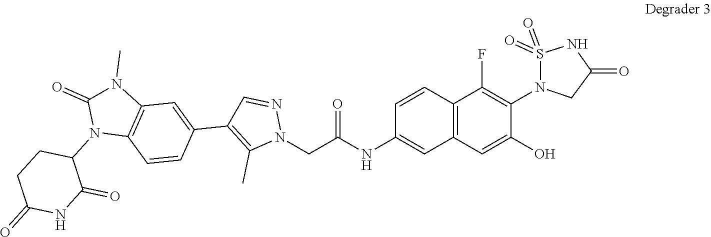 [SMILES: Cc1c(-c2ccc3c(c2)n(C)c(=O)n3C2CCC(=O)NC2=O)cnn1CC(=O)Nc1ccc2c(F)c(N3CC(=O)NS3(=O)=O)c(O)cc2c1]

2-[4-[1-(2,6-dioxo-3-piperidyl)-3-methyl-2-oxo-benzimidazol-5-yl]-5-methyl-pyrazol-1-yl]-N-[5-fluoro-7-hydroxy-6-(1,1,4-trioxo-1,2,5-thiadiazolidin-2-yl)-2-naphthyl]acetamide (Degrader 3, 22 mg, 27.96 Mol, formic acid salt) was synthesized following the same procedures as 2-[4-[1-(2,6-dioxo-3-piperidyl)-3-methyl-2-oxo-benzimidazol-5-yl]-3-methyl-pyrazol-1-yl]-N-[5-fluoro-7-hydroxy-6-(1,1,4-trioxo-1,2,5-thiadiazolidin-2-yl)-2-naphthyl]acetamide (Degrader 2)

LCMS (ES−): m/z 689 [M−H]⁻

¹H-NMR (400 MHz, DMSO-d6): δ 11.11 (s, 1H), 10.62 (s, 1H), 10.20 (s, 1H), 8.18 (s, 1H), 7.90 (d, J=8.80 Hz, 1H), 7.64 (s, 1H), 7.45 (dd, J=2.00, 9.00 Hz, 1H), 7.23 (s, 1H), 7.17 (d, J=8.00 Hz, 1H), 7.09 (d, J=6.80 Hz, 1H), 6.97 (s, 1H), 5.40 (dd, J=5.60, 12.60 Hz, 1H), 5.12 (s, 2H), 4.29 (s, 2H), 3.40 (s, 3H), 2.97-2.88 (m, 1H), 2.80-2.67 (m, 2H), 2.43 (s, 3H), 2.10-2.05 (m, 1H).

### 2-[4-[[1-(2,6-dioxo-3-piperidyl)-3-methyl-2-oxo-benzimidazol-5-yl]methyl]piperazin-1-yl]-N-[5-fluoro-7-hydroxy-6-(1,1,4-trioxo-1,2,5-thiadiazolidin-2-yl)-2-naphthyl]acetamide (Degrader 4)

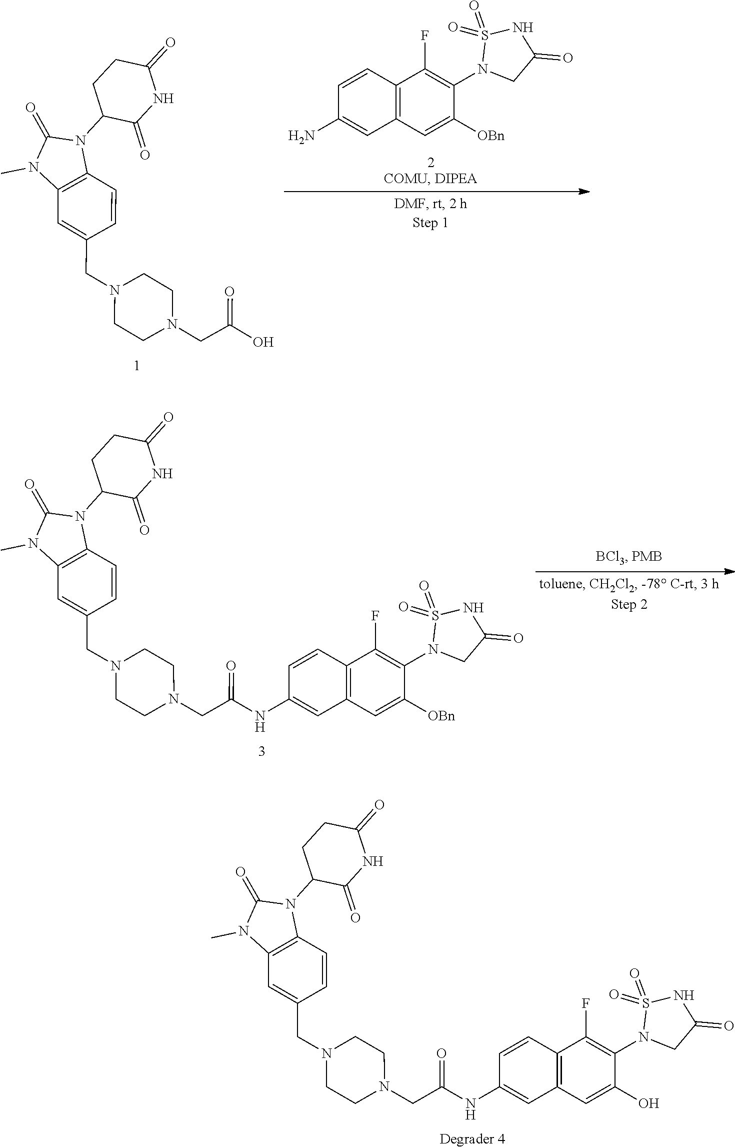 [SMILES: Cn1c(=O)n(C2CCC(=O)NC2=O)c2ccc(CN3CCN(CC(=O)O)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(CN3CCN(CC(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(CN3CCN(CC(=O)Nc4ccc5c(F)c(N6CC(=O)NS6(=O)=O)c(O)cc5c4)CC3)cc21]

### Step 1: N-[7-benzyloxy-5-fluoro-6-(1,1,4-trioxo-1,2,5-thiadiazolidin-2-yl)-2-naphthyl]-2-[4-[[1-(2,6-dioxo-3-piperidyl)-3-methyl-2-oxo-benzimidazol-5-yl]methyl]piperazin-1-yl]acetamide (3)

Into a 20 mL screw-capped vial containing a well-stirred solution of 2-[4-[[1-(2,6-dioxo-3-piperidyl)-3-methyl-2-oxo-benzimidazol-5-yl]methyl]piperazin-1-yl]acetic acid (1, 155 mg, 281.26 μmol, HCl salt) in DMF (2 mL) was added DIPEA (181.75 mg, 1.41 mmol, 244.94 μL) followed by COMU® (180.57 mg, 421.62 μmol) at room temperature. The reaction mixture was stirred at same temperature 10 min. Subsequently, 5-(6-amino-3-benzyloxy-1-fluoro-2-naphthyl)-1,1-dioxo-1,2,5-thiadiazolidin-3-one (2, 165.11 mg, 281.26 μmol, TFA salt) was added and the reaction mixture was stirred for 2 h. The solvent was removed under reduced pressure and the residue was diluted with ice water (8 mL) to get a solid that was filtered and dried to afford N-[7-benzyloxy-5-fluoro-6-(1,1,4-trioxo-1,2,5-thiadiazolidin-2-yl)-2-naphthyl]-2-[4-[[1-(2,6-dioxo-3-piperidyl)-3-methyl-2-oxo-benzimidazol-5-yl]methyl]piperazin-1l-yl]acetamide (3, 200 mg, 92.63 μmol, 33% yield) as off white solid.

LCMS (ES+): m/z 799.1 [M+H]⁺

### Step 2: 2-[4-[[1-(2,6-dioxo-3-piperidyl)-3-methyl-2-oxo-benzimidazol-5-yl]methyl]piperazin-1-yl]-N-[5-fluoro-7-hydroxy-6-(1,1,4-trioxo-1,2,5-thiadiazolidin-2-yl)-2-naphthyl]acetamide (Degrader 4)

Into a 25 mL round-bottom flask containing a well-stirred solution of N-[7-benzyloxy-5-fluoro-6-(1,1,4-trioxo-1,2,5-thiadiazolidin-2-yl)-2-naphthyl]-2-[4-[[1-(2,6-dioxo-3-piperidyl)-3-methyl-2-oxo-benzimidazol-5-yl]methyl]piperazin-1-yl]acetamide (3, 200 mg, 92.63 μmol) in DCM (2.5 mL) and toluene (2.5 mL) was added pentamethylbenzene (68.66 mg, 463.17 μmol) and the reaction mixture was cooled to −78° C. Then BCl₃ (1.0 M solution in DCM, 1.85 mmol, 1.84 mL) was added dropwise over a period of 2 min. Subsequently the reaction mixture was brought to room temperature and stirred for 3 h. The reaction mixture was cooled to −78° C. and quenched slowly with 5% dichloromethane in methanol (2.5 mL). The reaction mixture was allowed to attain room temperature and concentrated under reduced pressure to get the crude material that was purified by preparative HPLC [Column: SUNFIRE C18 (19×150) mm, 5 μm; Mobile Phase A: 0.1% HCOOH in water and Mobile Phase B: MeCN] to get 2-[4-[[1-(2,6-dioxo-3-piperidyl)-3-methyl-2-oxo-benzimidazol-5-yl]methyl]piperazin-1-yl]-N-[5-fluoro-7-hydroxy-6-(1,1,4-trioxo-1,2,5-thiadiazolidin-2-yl)-2-naphthyl]acetamide (Degrader 4, 10 mg, 13.07 μmol, 14% yield, formic acid salt) as an off-white solid.

LCMS (ES+): m/z 709.0 [M+H]⁺

¹H-NMR (400 MHz, DMSO-d6): δ 11.13 (s, 1H), 10.00 (s, 1H), 9.72 (s, 1H), 9.42 (brs, 1H), 8.11 (s, 1H), 7.85 (d, J=9.20 Hz, 1H), 7.49 (d, J=9.20 Hz, 1H), 7.33 (s, 1H), 7.24-7.21 (m, 2H), 6.95 (s, 1H), 5.44-5.41 (m, 1H), 4.38 (s, 2H), 4.08 (s, 2H), 3.54 (s, 3H), 3.38-2.88 (m, 6H), 2.77-2.68 (m, 6H), 2.10-1.09 (m, 1H).

### N-(6-(1,1-dioxido-4-oxo-1,2,5-thiadiazolidin-2-yl)-5-fluoro-7-hydroxynaphthalen-2-yl)-2-(5-(1-(2,6-dioxopiperidin-3-yl)-3-methyl-2-oxo-2,3-dihydro-1H-benzo[d]imidazol-5-yl)-2,5-diazabicyclo[2.2.2]octan-2-yl)acetamide (Degrader 5)

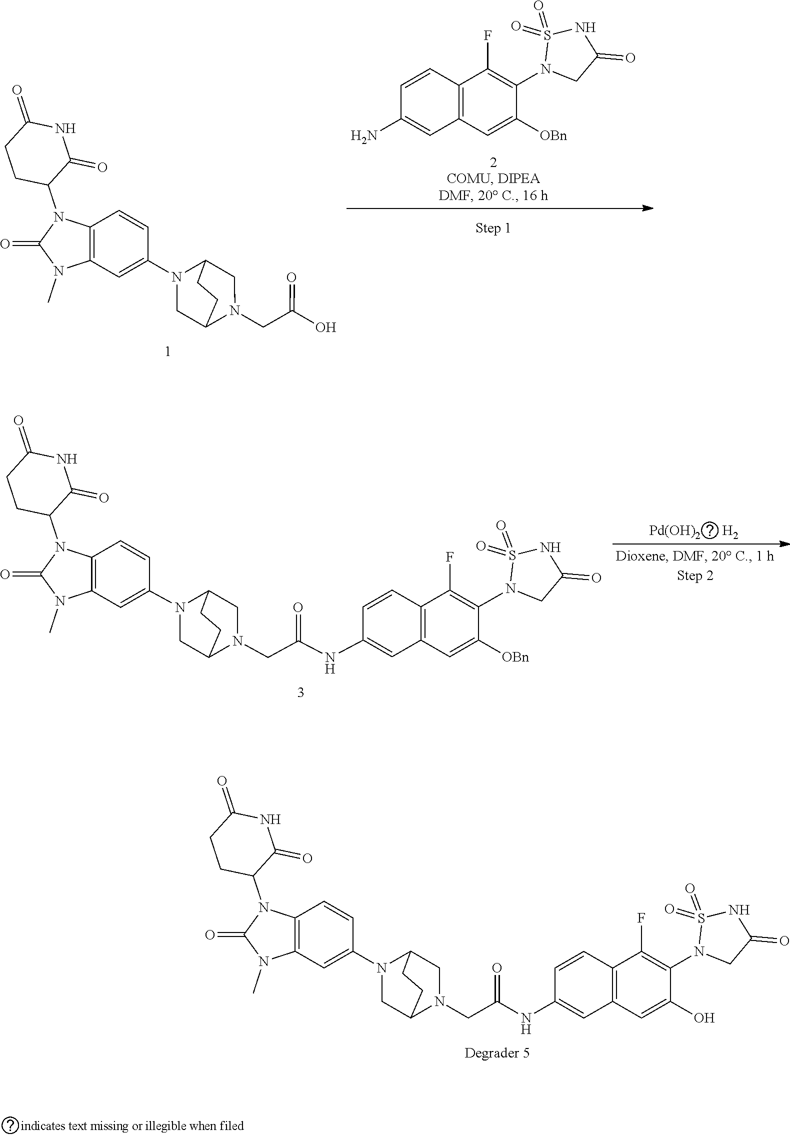 [SMILES: Cn1c(=O)n(C2CCC(=O)NC2=O)c2ccc(N3CC4CCC3CN4CC(=O)O)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(N3CC4CCC3CN4CC(=O)Nc3ccc4c(F)c(N5CC(=O)NS5(=O)=O)c(OCc5ccccc5)cc4c3)cc21 ; "Pd(OH)2" ; "H2" ; Cn1c(=O)n(C2CCC(=O)NC2=O)c2ccc(N3CC4CCC3CN4CC(=O)Nc3ccc4c(F)c(N5CC(=O)NS5(=O)=O)c(O)cc4c3)cc21]

### Step 1: N-(7-(benzyloxy)-6-(1,1-dioxido-4-oxo-1,2,5-thiadiazolidin-2-yl)-5-fluoronaphthalen-2-yl)-2-(5-(1-(2,6-dioxopiperidin-3-yl)-3-methyl-2-oxo-2,3-dihydro-1H-benzo[d]imidazol-5-yl)-2,5-diazabicyclo[2.2.2]octan-2-yl)acetamide (3)

To a mixture of 2-(5-(1-(2,6-dioxopiperidin-3-yl)-3-methyl-2-oxo-2,3-dihydro-1H-benzo[d]imidazol-5-yl)-2,5-diazabicyclo[2.2.2]octan-2-yl)acetic acid (1, 134 mg, 288.85 μmol, 1 eq, HCl salt), 5-(6-amino-3-(benzyloxy)-1-fluoronaphthalen-2-yl)-1,2,5-thiadiazolidin-3-one 1,1-dioxide (2, 148.88 mg, 288.85 μmol, 1 eq, TFA salt) and DIPEA (186.66 mg, 1.44 mmol, 251.56 μL, 5 eq) in DMF (3 mL) was added COMU (136.07 mg, 317.73 μmol, 1.1 eq), then the mixture was stirred at 20° C. for 16 h. The reaction mixture was purified by prep-HPLC (flow: 25 mL/min; gradient: from 21-51% MeCN in water (0.1% TFA); column: Phenomenex Synergi C18 150×25 mm×10 um) to afford N-(7-(benzyloxy)-6-(1,1-dioxido-4-oxo-1,2,5-thiadiazolidin-2-yl)-5-fluoronaphthalen-2-yl)-2-(5-(1-(2,6-dioxopiperidin-3-yl)-3-methyl-2-oxo-2,3-dihydro-1H-benzo[d]imidazol-5-yl)-2,5-diazabicyclo[2.2.2]octan-2-yl)acetamide (3, 150 mg, 160.56 μmol, 56% yield, TFA salt) as yellow solid.

LCMS (ESI): m/z 811.3 [M+H]⁺

### Step 2: N-(6-(1,1-dioxido-4-oxo-1,2,5-thiadiazolidin-2-yl)-5-fluoro-7-hydroxynaphthalen-2-yl)-2-(5-(1-(2,6-dioxopiperidin-3-yl)-3-methyl-2-oxo-2,3-dihydro-1H-benzo[d]imidazol-5-yl)-2,5-diazabicyclo[2.2.2]octan-2-yl)acetamide (Degrader 5)

To a solution of N-(7-(benzyloxy)-6-(1,1-dioxido-4-oxo-1,2,5-thiadiazolidin-2-yl)-5-fluoronaphthalen-2-yl)-2-(5-(1-(2,6-dioxopiperidin-3-yl)-3-methyl-2-oxo-2,3-dihydro-1H-benzo[d]imidazol-5-yl)-2,5-diazabicyclo[2.2.2]octan-2-yl)acetamide (3, 100 mg, 123.33 μmol, 1 eq) in DMF (2 mL) and 1,4-dioxane (2 mL) was added Pd/C (10 mg, 10% purity) and Pd(OH)₂/C (10 mg, 10% purity) under H₂, then the mixture was stirred at 20° C. for 1 h under H₂ (15 psi) atmosphere. The mixture was filtered and concentrated to remove dioxane. The residue was purified by prep-HPLC (flow: 25 mL/min; gradient: from 12-42% MeCN in water (0.1% TFA); column: 3_Phenomenex Luna C18 75×30 mm×3 um) to afford N-(6-(1,1-dioxido-4-oxo-1,2,5-thiadiazolidin-2-yl)-5-fluoro-7-hydroxynaphthalen-2-yl)-2-(5-(1-(2,6-dioxopiperidin-3-yl)-3-methyl-2-oxo-2,3-dihydro-1H-benzo[d]imidazol-5-yl)-2,5-diazabicyclo[2.2.2]octan-2-yl)acetamide (Degrader 5, 23.29 mg, 27.90 μmol, 23% yield, TFA salt) as gray solid.

LCMS (ESI): m/z 721.2 [M+H]⁺

¹H NMR (400 MHz, d₆-DMSO) δ 11.11-11.04 (m, 1H), 10.88-10.68 (m, 1H), 10.51-10.21 (m, 1H), 9.98 (br s, 1H), 8.19-8.04 (m, 1H), 7.96-7.84 (m, 1H), 7.51-7.38 (m, 1H), 7.03-6.91 (m, 2H), 6.68 (br s, 1H), 6.43 (br s, 1H), 5.36-5.22 (m, 1H), 4.58-4.45 (m, 1H), 4.37 (br d, J=1.4 Hz, 1H), 4.21 (br s, 1H), 4.14-4.09 (m, 2H), 3.93 (br d, J=2.6 Hz, 2H), 3.84-3.80 (m, 1H), 3.31 (br s, 5H), 2.90-2.86 (m, 1H), 2.68-2.64 (m, 1H), 2.26 (br s, 1H), 2.05-1.82 (m, 5H).

### 3-[[4-[1-(2,6-dioxo-3-piperidyl)-3-methyl-2-oxo-benzimidazol-5-yl]-1-piperidyl]methyl]-N-[5-fluoro-7-hydroxy-6-(1,1,4-trioxo-1,2,5-thiadiazolidin-2-yl)-2-naphthyl]cyclobutanecarboxamide (Degrader 6)

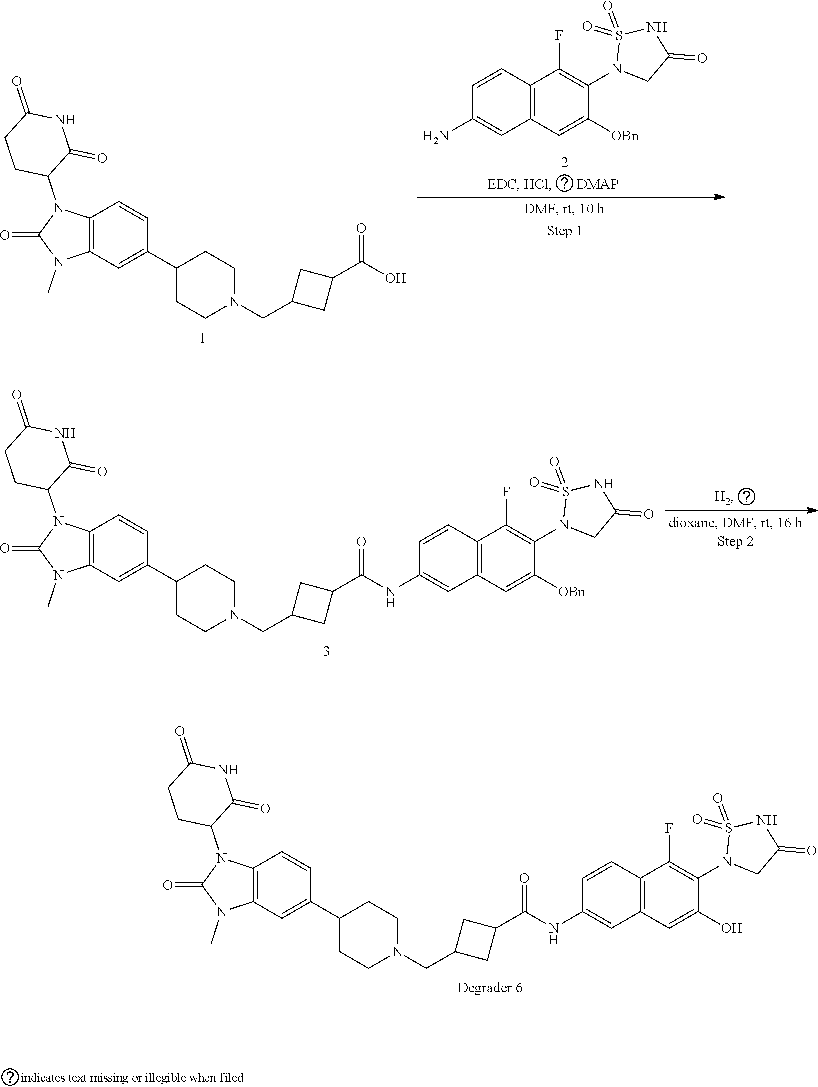 [SMILES: Cn1c(=O)n(C2CCC(=O)NC2=O)c2ccc(C3CCN(CC4CC(C(=O)O)C4)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(C3CCN(CC4CC(C(=O)Nc5ccc6c(F)c(N7CC(=O)NS7(=O)=O)c(OCc7ccccc7)cc6c5)C4)CC3)cc21 ; Cn1c(=O)n(C2CCC(=O)NC2=O)c2ccc(C3CCN(CC4CC(C(=O)Nc5ccc6c(F)c(N7CC(=O)NS7(=O)=O)c(O)cc6c5)C4)CC3)cc21]

### Step 1: N-[7-benzyloxy-5-fluoro-6-(1,1,4-trioxo-1,2,5-thiadiazolidin-2-yl)-2-naphthyl]-3-[[4-[1-(2,6-dioxo-3-piperidyl)-3-methyl-2-oxo-benzimidazol-5-yl]-3,6-dihydro-2H-pyridin-1-yl]methyl]cyclobutanecarboxamide (3)

Into a 25 mL single neck round bottom flask containing a well-stirred solution of 3-[[4-[1-(2,6-dioxo-3-piperidyl)-3-methyl-2-oxo-benzimidazol-5-yl]-1-piperidyl]methyl]cyclobutanecarboxylic acid (1, 250 mg, 473.54 μmol, HCl salt) and 5-(6-amino-3-benzyloxy-1-fluoro-2-naphthyl)-1,1-dioxo-1,2,5-thiadiazolidin-3-one (2, 208.89 mg, 473.54 μmol, TFA salt) in anhydrous DMF (0.5 mL) were added at room temperature HOBt (127.97 mg, 947.09 μmol), DMAP (289.26 mg, 2.37 mmol) and N-(3-dimethylaminopropyl)-N′-ethylcarbodiimide hydrochloride (272.34 mg, 1.42 mmol). The resulting mixture was stirred at room temperature for 16 h and then treated with ice-cold water. The precipitate was filtered, dried and purified by reverse-phase column chromatography [Column: Redisep C18 120 g; Mobile Phase A: 0.1% Formic acid in water and Mobile Phase B: MeCN] to afford N-[7-benzyloxy-5-fluoro-6-(1,1,4-trioxo-1,2,5-thiadiazolidin-2-yl)-2-naphthyl]-3-[[4-[1-(2,6-dioxo-3-piperidyl)-3-methyl-2-oxo-benzimidazol-5-yl]-3,6-dihydro-2H-pyridin-1-yl]methyl]cyclobutanecarboxamide (3, 205 mg, 225.63 μmol, 48% yield) as a beige solid.

LCMS (ES+): m/z 838.3 [M+H]⁺

### Step 2: 3-[[4-[1-(2,6-dioxo-3-piperidyl)-3-methyl-2-oxo-benzimidazol-5-yl]-1-piperidyl]methyl]-N-[5-fluoro-7-hydroxy-6-(1,1,4-trioxo-1,2,5-thiadiazolidin-2-yl)-2-naphthyl]cyclobutanecarboxamide (Degrader 6)

Into a 50 mL single neck round bottom flask containing a well-stirred solution of N-[7-benzyloxy-5-fluoro-6-(1,1,4-trioxo-1,2,5-thiadiazolidin-2-yl)-2-naphthyl]-3-[[4-[1-(2,6-dioxo-3-piperidyl)-3-methyl-2-oxo-benzimidazol-5-yl]-1-piperidyl]methyl]cyclobutanecarboxamide (3, 200 mg, 208.16 μmol, formic acid salt) in anhydrous 1,4-dioxane (2.0 mL) and DMF (0.9 mL) was added palladium hydroxide on carbon (20 wt. %) (200 mg, 208.16 μmol). The resultant reaction mixture was stirred under hydrogen bladder pressure for 16 h. The reaction mixture was filtered through Celite and washed with a mixture of 1,4-dioxane (20 mL) and DMF (7 mL). The filtrate was evaporated under reduced pressure to afford crude material that was purified by reverse-phase preparative HPLC [Column: SUNFIRE C18 (19×150) mm, 5 μm; Mobile Phase A: 0.1% Formic acid in water and Mobile phase B: Acetonitrile] to afford 3-[[4-[1-(2,6-dioxo-3-piperidyl)-3-methyl-2-oxo-benzimidazol-5-yl]-1-piperidyl]methyl]-N-[5-fluoro-7-hydroxy-6-(1,1,4-trioxo-1,2,5-thiadiazolidin-2-yl)-2-naphthyl]cyclobutanecarboxamide (Degrader 6, 20 mg, 24.82 μmol, 12% yield, formic acid salt).

LCMS (ES−): m/z 746.0 [M−H]⁻

¹H-NMR (400 MHz, DMSO-d6): δ 11.18 (s, 1H), 10.04 (s, 1H), 9.72 (s, 1H), 8.17 (t, J=6.80 Hz, 1H), 7.83 (d, J=9.20 Hz, 1H), 7.44 (d, J=9.20 Hz, 1H), 7.08-7.06 (m, 2H), 6.94-6.91 (m, 2H), 5.36 (dd, J=5.60, 14.00 Hz, 1H), 4.08 (s, 2H), 3.60-3.40 (m, 2H), 3.33 (s, 3H), 3.30-3.10 (m, 2H), 3.04-2.80 (m, 4H), 2.81-2.60 (m, 3H), 2.45-2.35 (m, 2H), 2.21-1.75 (m, 8H).

### 3-[5-[1-[5-[[8-fluoro-6-hydroxy-7-(1,1,4-trioxo-1,2,5-thiadiazolidin-2-yl)-2-naphthyl]oxy]-3,3-dimethyl-pentyl]-4-piperidyl]-3-methyl-2-oxo-benzimidazol-1-yl]piperidine-2,6-dione (Degrader 7)

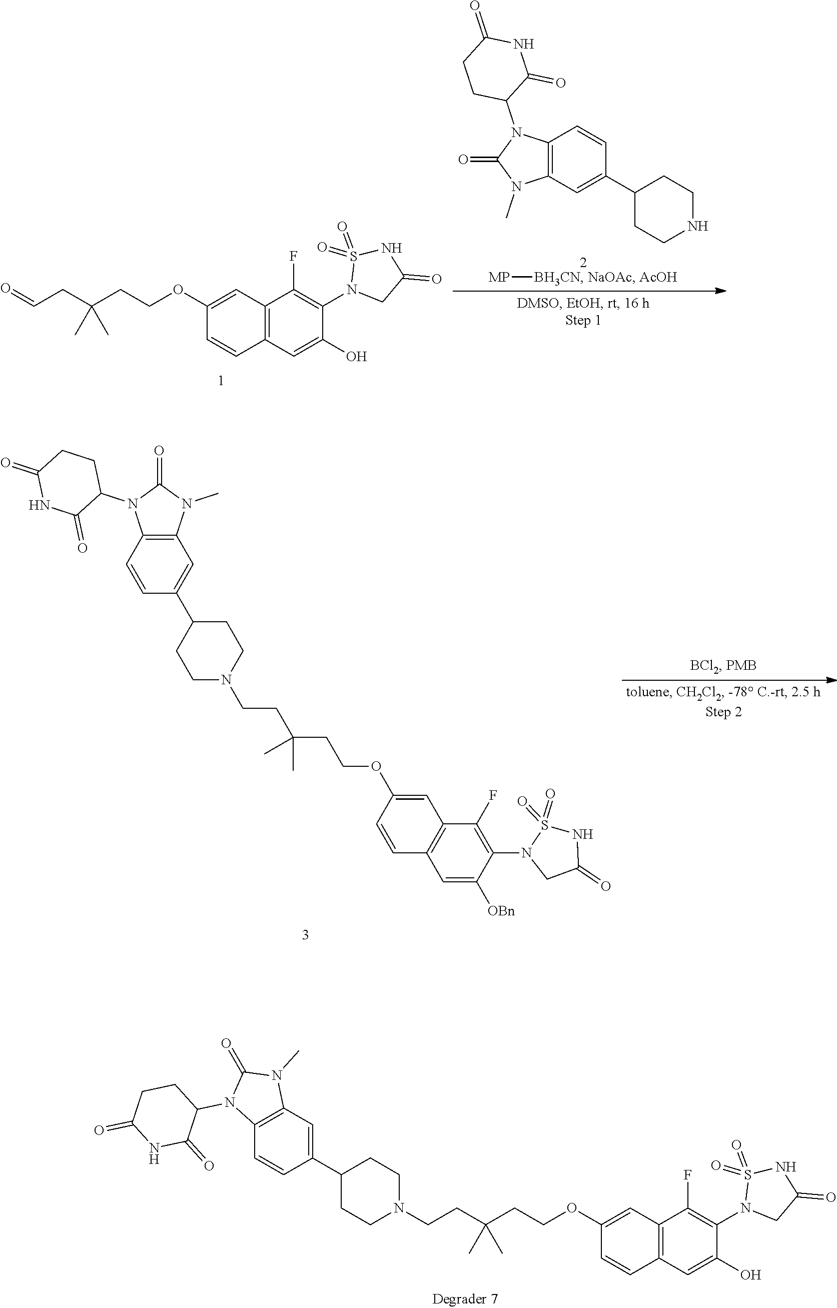

### Step 1: 3-[5-[1-[5-[[6-benzyloxy-8-fluoro-7-(1,1,4-trioxo-1,2,5-thiadiazolidin-2-yl)-2-naphthyl]oxy]-3,3-dimethyl-pentyl]-4-piperidyl]-3-methyl-2-oxo-benzimidazol-1-yl]piperidine-2,6-dione (3)

Into a 100 mL single-neck, round-bottom flask containing a well-stirred solution of 5-[[6-benzyloxy-8-fluoro-7-(1,1,4-trioxo-1,2,5-thiadiazolidin-2-yl)-2-naphthyl]oxy]-3,3-dimethyl-pentanal (1, 350 mg, 302.00 μmol) in DMSO (2 mL) and EtOH (2 mL) was added 3-[3-methyl-2-oxo-5-(4-piperidyl)benzimidazol-1-yl]piperidine-2,6-dione (2, 143.58 mg, 302.00 μmol, TFA Salt), anhydrous NaOAc (74.32 mg, 906.01 μmol) and acetic acid (272.03 mg, 4.53 mmol, 259.08 uL). The resulting mixture was stirred at room temperature for 20 min. Biotage® MP-Cyano borohydride (2 mmol in 1 g, 302.21 mg, 604.53 μmol) was added and the resulting suspension was stirred at room temperature for 16 h. The reaction mixture was filtered and the filtrate was concentrated under vacuum to get the crude material that was purified by reverse-phase column chromatography [Column: SiliaSep premium C18, 25 um, 120 g; Mobile Phase: 0.1% TFA in Water/CH₃CN] to afford 3-[5-[1-[5-[[6-benzyloxy-8-fluoro-7-(1,1,4-trioxo-1,2,5-thiadiazolidin-2-yl)-2-naphthyl]oxy]-3,3-dimethyl-pentyl]-4-piperidyl]-3-methyl-2-oxo-benzimidazol-1-yl]piperidine-2,6-dione (3, 95 mg, 84.73 μmol, 28% yield, TFA salt) as an off-white solid.

LCMS (ES+): m/z 841.2 [M+H]⁺

### Step 2: 3-[5-[1-[5-[[8-fluoro-6-hydroxy-7-(1,1,4-trioxo-1,2,5-thiadiazolidin-2-yl)-2-naphthyl]oxy]-3,3-dimethyl-pentyl]-4-piperidyl]-3-methyl-2-oxo-benzimidazol-1-yl]piperidine-2,6-dione (Degrader 7)

Into a 50 mL single-neck, round-bottom flask containing a well-stirred solution of 3-[5-[1-[5-[[6-benzyloxy-8-fluoro-7-(1,1,4-trioxo-1,2,5-thiadiazolidin-2-yl)-2-naphthyl]oxy]-3,3-dimethyl-pentyl]-4-piperidyl]-3-methyl-2-oxo-benzimidazol-1-yl]piperidine-2,6-dione (3, 95 mg, 96.21 μmol, TFA salt) in anhydrous DCM (3.0 mL) and anhydrous toluene (3.0 mL) was added pentamethylbenzene (42.79 mg, 288.64 μmol) at room temperature. The reaction mixture was cooled to −78° C. and to this was added BCl₃ (1.0 M solution in DCM, 1.92 mmol, 2.0 mL). The reaction mixture was subsequently brought to room temperature and stirred for 2.5 h. The reaction mixture was quenched with 5% MeOH in DCM (1.2 mL) at −78° C. The reaction mixture was concentrated under reduced pressure to get the crude material that was purified by reverse-phase preparative HPLC [Column: X-bridge, C18 (150×19) mm, 5 μm; Mobile phase A: 0.1% Formic acid in water and Mobile phase B: CH₃CN] to afford 3-[5-[1-[5-[[8-fluoro-6-hydroxy-7-(1,1,4-trioxo-1,2,5-thiadiazolidin-2-yl)-2-naphthyl]oxy]-3,3-dimethyl-pentyl]-4-piperidyl]-3-methyl-2-oxo-benzimidazol-1-yl]piperidine-2,6-dione (Degrader 7, 41 mg, 49.99 μmol, 52% yield, Formic acid salt) as a white solid.

LCMS (ES+): m/z 751.2 [M+H]⁺

¹H-NMR (400 MHz, DMSO-d6): δ 11.09 (s, 1H), 9.46 (brs, 1H), 8.29 (s, 1H), 7.67 (d, J=9.20 Hz, 1H), 7.23 (s, 1H), 7.12 (d, J=8.80 Hz, 1H), 7.09 (s, 1H), 7.03 (s, 1H), 6.99 (d, J=8.00 Hz, 1H), 6.89 (d, J=8.00 Hz, 1H), 5.33 (dd, J=4.80, 12.40 Hz, 1H), 4.14 (s, 2H), 4.09 (s, 2H), 3.10-2.85 (m, 3H), 2.80-2.60 (m, 2H), 2.39-2.34 (m, 3H), 2.06-2.01 (m, 3H), 1.75-1.68 (m, 6H), 1.52-1.50 (m, 2H), 1.00 (s, 6H).

### N-(6-(1,1-dioxido-4-oxo-1,2,5-thiadiazolidin-2-yl)-5-fluoro-7-hydroxynaphthalen-2-yl)-3-(1-(1-(2,6-dioxopiperidin-3-yl)-3-methyl-2-oxo-2,3-dihydro-1H-benzo[d]imidazol-5-yl)piperidin-4-yl)propanamide (Degrader 8)

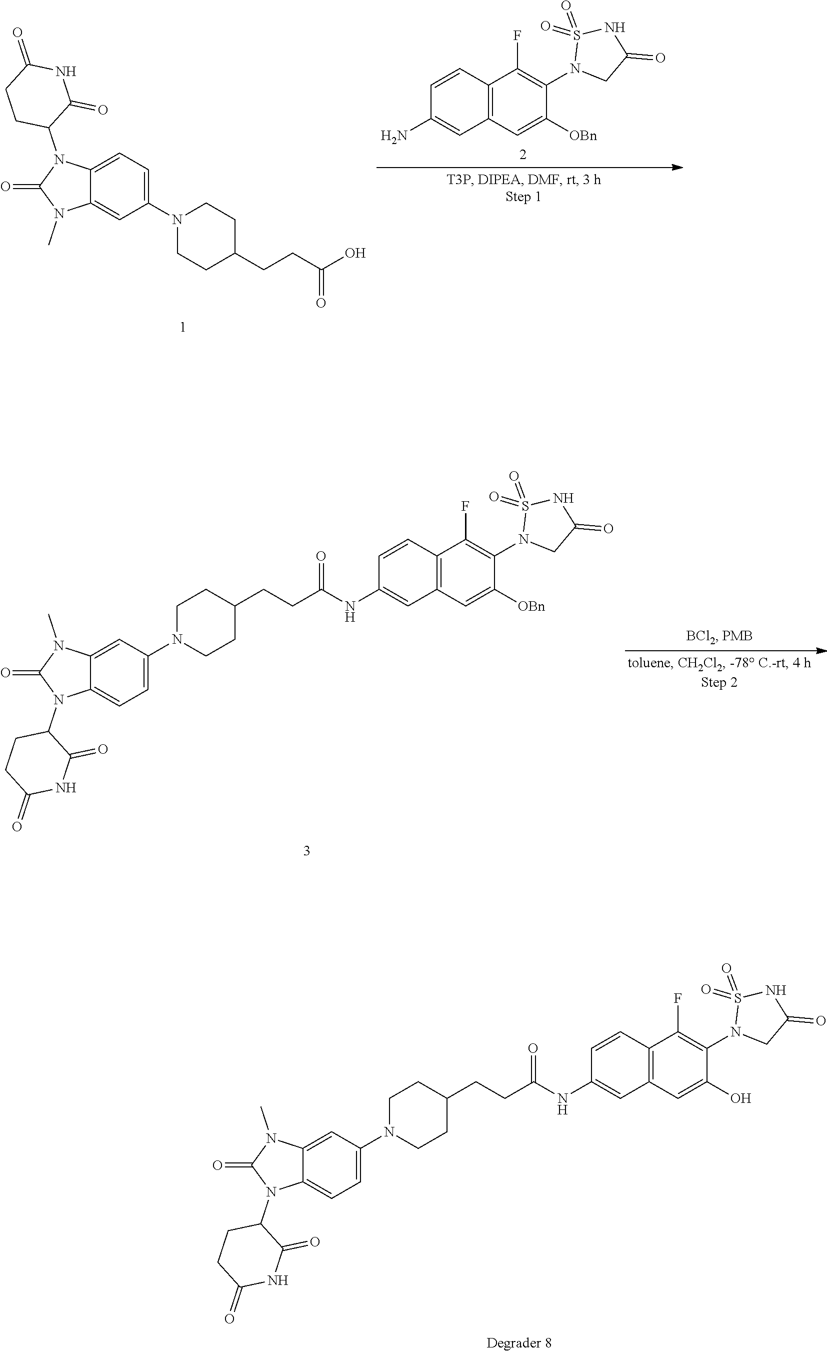 [SMILES: Cn1c(=O)n(C2CCC(=O)NC2=O)c2ccc(N3CCC(CCC(=O)O)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(N3CCC(CCC(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(N3CCC(CCC(=O)Nc4ccc5c(F)c(N6CC(=O)NS6(=O)=O)c(O)cc5c4)CC3)cc21]

### Step 1: N-(7-(benzyloxy)-6-(1,1-dioxido-4-oxo-1,2,5-thiadiazolidin-2-yl)-5-fluoronaphthalen-2-yl)-3-(1-(1-(2,6-dioxopiperidin-3-yl)-3-methyl-2-oxo-2,3-dihydro-1H-benzo[d]imidazol-5-yl) piperidin-4-yl) procainamide (3)

Into a 25 mL single-neck, round-bottom flask containing a well-stirred solution of 3-[1-[1-(2,6-dioxo-3-piperidyl)-3-methyl-2-oxo-benzimidazol-5-yl]-4-piperidyl] propionic acid (1, 120.83 mg, 291.55 μmol, TFA salt) in DMF (4 mL) at room temperature, was added DIPEA (113.04 mg, 874.65 μmol, 152.34 μL) and propylphosphonic anhydride solution (>50 wt. % in EtOAc, 278.30 mg, 437.32 μmol) at rt. The reaction mixture was stirred at room temperature for 1 h. Subsequently, 5-(6-amino-3-benzyloxy-1-fluoro-2-naphthyl)-1,1-dioxo-1,2,5-thiadiazolidin-3-one (2, 150.27 mg, 291.55 μmol, TFA salt) was added and the reaction mixture was stirred for an additional 3 h. The reaction mixture was concentrated, washed with ice cold water and dried under vacuum to obtain the crude N-(7-(benzyloxy)-6-(1,1-dioxido-4-oxo-1,2,5-thiadiazolidin-2-yl)-5-fluoronaphthalen-2-yl)-3-(1-(1-(2,6-dioxopiperidin-3-yl)-3-methyl-2-oxo-2,3-dihydro-1H-benzo[d]imidazol-5-yl) piperidin-4-yl) procainamide (3, 0.28 g, 167.05 μmol, 57% yield) as an off-white solid.

LCMS (ES+): m/z 798.2 [M+H]⁺

### Step 2: N-(6-(1,1-dioxido-4-oxo-1,2,5-thiadiazolidin-2-yl)-5-fluoro-7-hydroxynaphthalen-2-yl)-3-(1-(1-(2,6-dioxopiperidin-3-yl)-3-methyl-2-oxo-2,3-dihydro-1H-benzo[d]imidazol-5-yl)piperidin-4-yl)propanamide (Degrader 8)

Into a 25 mL single-neck, round-bottom flask containing a well-stirred solution of N-[7-benzyloxy-5-fluoro-6-(1,1,4-trioxo-1,2,5-thiadiazolidin-2-yl)-2-naphthyl]-3-[1-[1-(2,6-dioxo-3-piperidyl)-3-methyl-2-oxo-benzimidazol-5-yl]-4-piperidyl] procainamide (3, 0.28 g, 350.94 μmol) in a 1:1 mixture of anhydrous toluene (10 mL) and DCM (10 mL) was added pent methylbenzene (260.13 mg, 1.75 mmol, 283.67 μL) at ambient temperature under nitrogen atmosphere. The reaction mixture was then cooled to −78° C. and Boron trichloride solution (1.0 M in methylene dichloride, 7.02 mmol, 7.0 mL) was added dropwise. The resulting mixture was allowed to stir at ambient temperature for 4 h. The reaction mixture was again cooled to −78° C. and quenched with 5% MeOH/DCM (1 mL). The reaction mixture was concentrated under reduced pressure to get the crude material that was subjected to purification by a reverse-phase preparative HPLC (Column; XBRIDGE C18 (30×150) mm, 5 μm; Mobile Phase A: 0.1% Formic acid in Water and Mobile Phase B: CH₃CN) to afford N-(6-(1,1-dioxido-4-oxo-1,2,5-thiadiazolidin-2-yl)-5-fluoro-7-hydroxynaphthalen-2-yl)-3-(1-(1-(2,6-dioxopiperidin-3-yl)-3-methyl-2-oxo-2,3-dihydro-1H-benzo[d]imidazol-5-yl)piperidin-4-yl)propanamide (Degrader 8, 40 mg, 51.50 μmol, 15% yield, formic acid salt) as an off-white powder.

LCMS (ES+): m/z 708.8 [M+H]⁺

¹H-NMR (400 MHz, DMSO-d6): δ 11.12 (s, 1H), 10.17 (s, 1H), 9.91 (brs, 1H), 8.18 (s, 1H), 7.84 (d, J=8.80 Hz, 1H), 7.44 (d, J=9.20 Hz, 1H), 7.21-7.08 (m, 3H), 6.95 (s, 1H), 5.45-5.33 (m, 1H), 4.16 (s, 2H), 3.63-3.60 (m, 2H), 3.35 (s, 3H), 2.94-2.86 (m, 2H), 2.74-2.65 (m, 5H), 2.02-1.96 (m, 3H), 1.72-1.39 (m, 5H).

### N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-(oxetan-3-yl)-2-oxo-2,3-dihydro-1H-benzo[d]imidazol-5-yl)piperidin-1-yl)acetamide (Degrader 9)

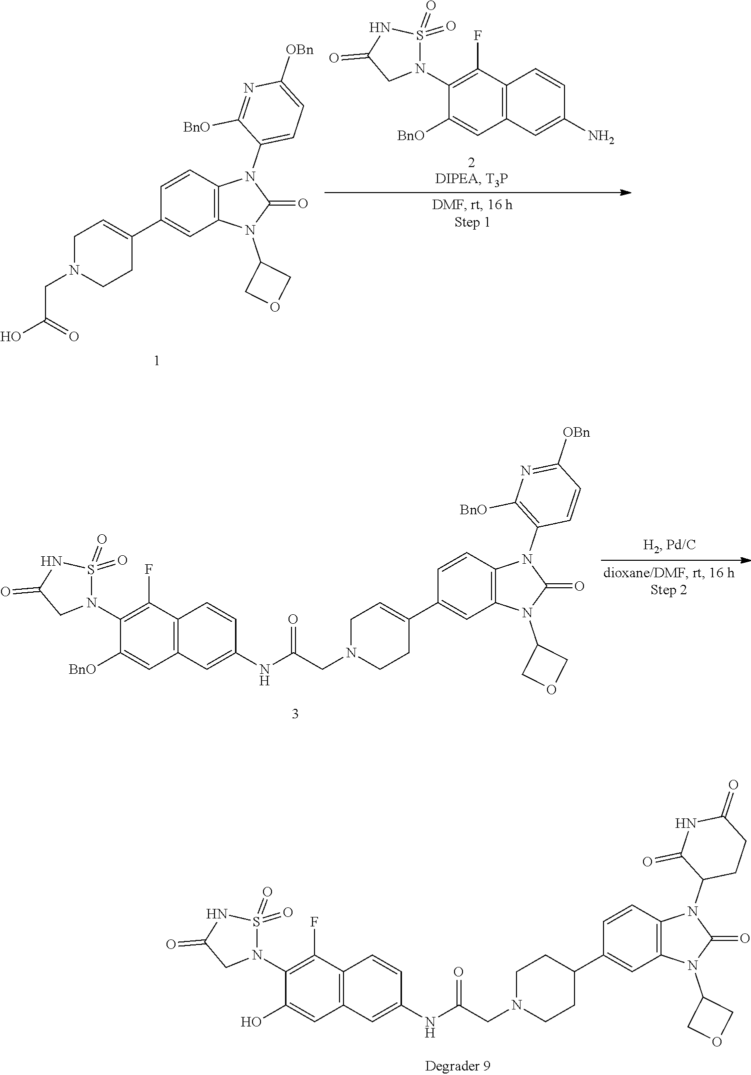

### Step 1: N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-(oxetan-3-yl)-2-oxo-2,3-dihydro-1H-benzo[d]imidazol-5-yl)piperidin-1-yl)acetamide (3)

To a solution of 2-(4-(1-(2,6-bis(benzyloxy)pyridin-3-yl)-3-(oxetan-3-yl)-2-oxo-2,3-dihydro-1H-benzo[d]imidazol-5-yl)-5,6-dihydropyridin-1 (2H)-yl)acetic acid (1, 200 mg, 323.27 μmol, 1 eq) and 5-(6-amino-3-(benzyloxy)-1-fluoronaphthalen-2-yl)-1,2,5-thiadiazolidin-3-one 1,1-dioxide (2, 129.76 mg, 323.27 μmol, 1 eq) in DMF (2 mL) were added DIPEA (626.71 mg, 4.85 mmol, 844.62 μL, 15 eq) and T₃P (1.44 g, 2.26 mmol, 50% purity in ethyl acetate, 7 eq). The mixture was stirred at 20° C. for 16 h. The reaction mixture was diluted with water (10 mL) and extracted with ethyl acetate (10 mL×3). The combined organic layers were washed with brine (20 mL), dried over Na₂SO₄, filtered and concentrated under reduced pressure to give residue. (SiO₂, ethyl acetate: ethyl alcohol=5:1) to give 170 mg product, which was purified by prep-HPLC (flow: 25 mL/min; gradient: from 17-47% MeCN-water (0.05% ammonia hydroxide v/v) over 7.5 min; column: Phenomenex Gemini-NX C18 75×30 mm×3 um) to afford N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-(oxetan-3-yl)-2-oxo-2,3-dihydro-1H-benzo[d]imidazol-5-yl)-5,6-dihydropyridin-1 (2H)-yl)acetamide (3, 110 mg, 109.77 μmol, 34% yield) as a yellow solid.

LCMS (ESI): m/z 1002.5 [M+H]⁺

### Step 2: N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-(oxetan-3-yl)-2-oxo-2,3-dihydro-1H-benzo[d]imidazol-5-yl)piperidin-1-yl)acetamide (Degrader 9)

To a solution of N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-(oxetan-3-yl)-2-oxo-2,3-dihydro-1H-benzo[d]imidazol-5-yl)-5,6-dihydropyridin-1 (2H)-yl)acetamide (3, 110 mg, 109.55 μmol, 1 eq) in dioxane (0.5 mL) and DMF (0.5 mL) was added Pd/C (75 mg) under N2 atmosphere. The mixture was degassed and purged with H₂ 3 times. Then the mixture was stirred under H₂ (15 Psi) at 20° C. for 16 h. The reaction mixture was filtered and concentrated under reduced pressure to give a residue. The residue was purified by prep-HPLC (flow: 30 mL/min; gradient: from 8-38% MeCN-water (10 mM NH₄HCO₃) over 2 min; column: Phenomenex Gemini-NX C18 75×30 mm×3 um) to afford N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-(oxetan-3-yl)-2-oxo-2,3-dihydro-1H-benzo[d]imidazol-5-yl)piperidin-1-yl)acetamide (Degrader 9, 32.92 mg, 44.30 μmol, 40% yield) as a pink solid.

LCMS (ESI): m/z 736.2 [M+H]⁺

¹H NMR (400 MHz, d₆-DMSO) δ 11.16-11.09 (m, 1H), 10.78 (s, 1H), 10.01-9.65 (m, 2H), 8.12 (s, 1H), 7.94-7.82 (m, 1H), 7.54-7.42 (m, 2H), 7.16-6.98 (m, 3H), 5.61-5.49 (m, 1H), 5.42-5.33 (m, 1H), 5.13-4.97 (m, 4H), 4.28-4.16 (m, 1H), 4.08 (s, 2H), 3.68 (s, 1H), 3.28-3.24 (m, 2H), 2.99-2.82 (m, 2H), 2.78-2.55 (m, 3H), 2.00 (s, 6H)

### 3-(5-(1-(2-(4-((7-(1,1-dioxido-4-oxo-1,2,5-thiadiazolidin-2-yl)-8-fluoro-6-hydroxynaphthalen-2-yl)oxy)piperidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (Degrader 10)

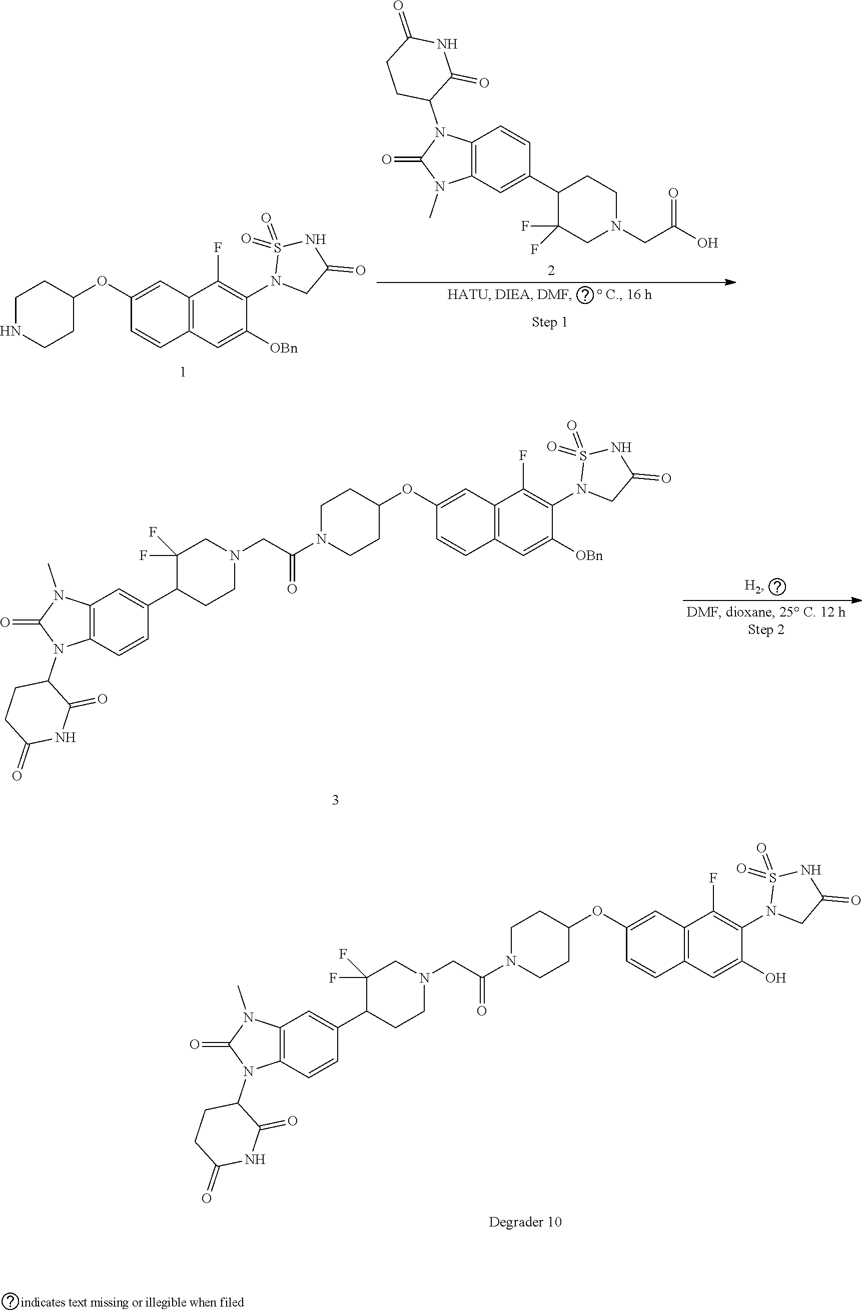 [SMILES: O=C1CN(c2c(OCc3ccccc3)cc3ccc(OC4CCNCC4)cc3c2F)S(=O)(=O)N1 ; Cn1c(=O)n(C2CCC(=O)NC2=O)c2ccc(C3CCN(CC(=O)O)CC3(F)F)cc21 ; Cn1c(=O)n(C2CCC(=O)NC2=O)c2ccc(C3CCN(CC(=O)N4CCC(Oc5ccc6cc(OCc7ccccc7)c(N7CC(=O)NS7(=O)=O)c(F)c6c5)CC4)CC3(F)F)cc21 ; Cn1c(=O)n(C2CCC(=O)NC2=O)c2ccc(C3CCN(CC(=O)N4CCC(Oc5ccc6cc(O)c(N7CC(=O)NS7(=O)=O)c(F)c6c5)CC4)CC3(F)F)cc21]

### Step 1: 3-(5-(1-(2-(4-((6-(benzyloxy)-7-(1,1-dioxido-4-oxo-1,2,5-thiadiazolidin-2-yl)-8-fluoronaphthalen-2-yl)oxy)piperidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (3)

To a solution of 2-(4-(1-(2,6-dioxopiperidin-3-yl)-3-methyl-2-oxo-2,3-dihydro-1H-benzo[d]imidazol-5-yl)-3,3-difluoropiperidin-1-yl)acetic acid (2, 180.00 mg, 412.46 μmol) in DMF (5 mL) was added N-ethyl-N-isopropylpropan-2-amine (266.19 mg, 2.06 mmol, 358.74 μL) and HATU (188 mg, 494.44 μmol). Then 5-(3-(benzyloxy)-1-fluoro-7-(piperidin-4-yloxy)naphthalen-2-yl)-1,2,5-thiadiazolidin-3-one 1,1-dioxide (1, 200 mg, 411.92 μmol) was added to the mixture. The mixture was stirred at 25° C. for 16 h. The reaction mixture was concentrated in vacuo. The residue was purified by reversed phase column (C18, 80 g; 0.1% formic acid in water/acetonitrile) to give 3-(5-(1-(2-(4-((6-(benzyloxy)-7-(1,1-dioxido-4-oxo-1,2,5-thiadiazolidin-2-yl)-8-fluoronaphthalen-2-yl)oxy)piperidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (3, 150 mg, 156.32 μmol, 38% yield, formic acid salt) as a white solid.

LCMS (ESI): m/z 904.3 [M+H]⁺

### Step 2: 3-(5-(1-(2-(4-((7-(1,1-dioxido-4-oxo-1,2,5-thiadiazolidin-2-yl)-8-fluoro-6-hydroxynaphthalen-2-yl)oxy)piperidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (Degrader 10)

To a solution of 3-(5-(1-(2-(4-((6-(benzyloxy)-7-(1,1-dioxido-4-oxo-1,2,5-thiadiazolidin-2-yl)-8-fluoronaphthalen-2-yl)oxy)piperidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (3, 140 mg, 154.88 μmol) in dioxane (3 mL) and DMF (3 mL) was added dihydroxypalladium (70.00 mg, 99.69 μmol, 20% purity) under N₂. The mixture was stirred under hydrogen (15 psi) at 25° C. for 12 h. The resulting mixture was filtered over Celite, and the filtrate was concentrated under reduced pressure. The residue was purified by prep-HPLC (flow: 25 mL/min, gradient: from 16-46%, water (0.225% TFA) in MeCN over 10 min; column: Phenomenex luna C18 150×25 mm×10 μm) to afford 3-(5-(1-(2-(4-((7-(1,1-dioxido-4-oxo-1,2,5-thiadiazolidin-2-yl)-8-fluoro-6-hydroxynaphthalen-2-yl)oxy)piperidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (Degrader 10, 79.53 mg, 79.72 μmol, 51% yield, TFA salt) as a off-white solid.

LCMS (ESI): m/z 814.5 [M+H]⁺

¹H NMR (400 MHz, DMSO-d₆) δ=11.11 (s, 1H), 10.13-9.84 (m, 1H), 7.72 (d, J=8.8 Hz, 1H), 7.33 (s, 1H), 7.21 (d, J=8.4 Hz, 1H), 7.15-7.08 (m, 2H), 7.06 (s, 1H), 7.00 (d, J=8.0 Hz, 1H), 5.38 (dd, J=5.2, 12.4 Hz, 1H), 4.85 (s, 1H), 4.31 (s, 2H), 4.24-4.03 (m, 2H), 3.99-3.84 (m, 2H), 3.83-3.73 (m, 2H), 3.44-3.31 (m, 8H), 3.25-3.01 (m, 2H), 2.98-2.85 (m, 1H), 2.75-2.59 (m, 2H), 2.16-1.94 (m, 4H), 1.84-1.52 (m, 2H).

### 2-[4-[3-(2,6-dioxo-3-piperidyl)-1-ethyl-indazol-6-yl]-3,3-difluoro-1-piperidyl]-N-[5-fluoro-7-hydroxy-6-(1,1,4-trioxo-1,2,5-thiadiazolidin-2-yl)-2-naphthyl]acetamide (Degrader 11)

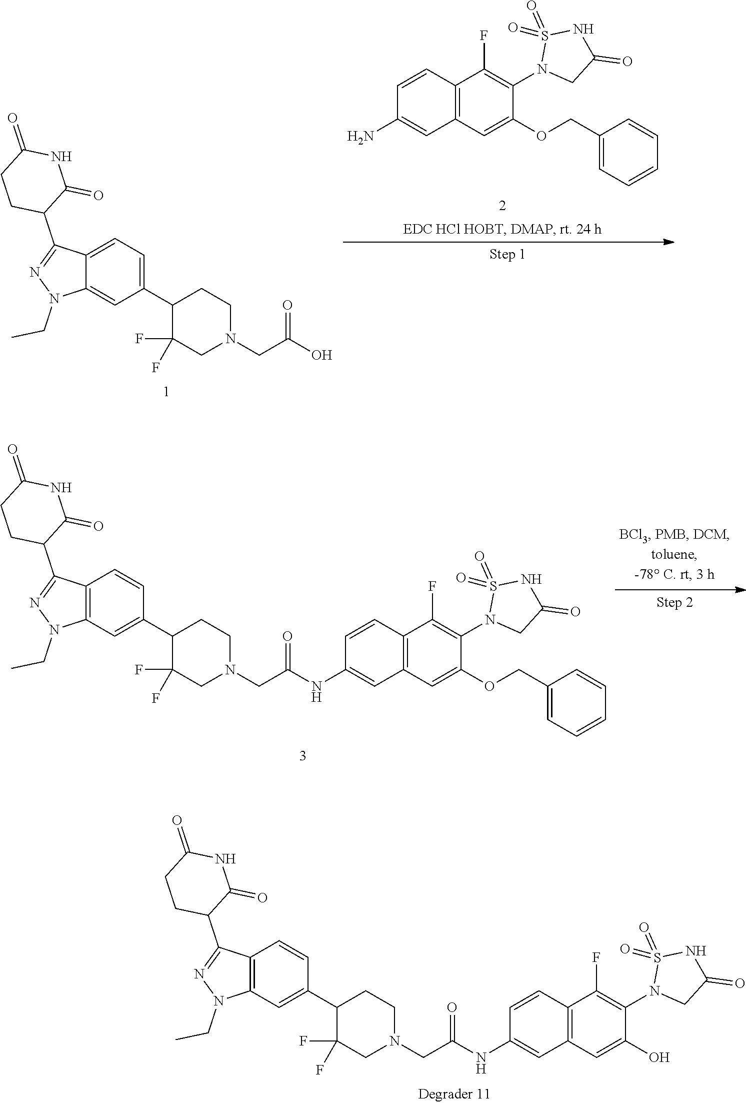 [SMILES: CCn1nc(C2CCC(=O)NC2=O)c2ccc(C3CCN(CC(=O)O)CC3(F)F)cc21 ; Nc1ccc2c(F)c(N3CC(=O)NS3(=O)=O)c(OCc3ccccc3)cc2c1 ; CCn1nc(C2CCC(=O)NC2=O)c2ccc(C3CCN(CC(=O)Nc4ccc5c(F)c(N6CC(=O)NS6(=O)=O)c(OCc6ccccc6)cc5c4)CC3(F)F)cc21 ; CCn1nc(C2CCC(=O)NC2=O)c2ccc(C3CCN(CC(=O)Nc4ccc5c(F)c(N6CC(=O)NS6(=O)=O)c(O)cc5c4)CC3(F)F)cc21]

### Step 1: N-[7-benzyloxy-5-fluoro-6-(1,1,4-trioxo-1,2,5-thiadiazolidin-2-yl)-2-naphthyl]-2-[4-[3-(2,6-dioxo-3-piperidyl)-1-ethyl-indazol-6-yl]-3,3-difluoro-1-piperidyl]acetamide (3)

Into a 50 mL single-neck round-bottom flask containing a well-stirred solution of 2-[4-[3-(2,6-dioxo-3-piperidyl)-1-ethyl-indazol-6-yl]-3,3-difluoro-1-piperidyl]acetic acid (1, 100 mg, 167.69 μmol, TFA salt) and 5-(6-amino-3-benzyloxy-1-fluoro-2-naphthyl)-1,1-dioxo-1,2,5-thiadiazolidin-3-one (2, 89.66 mg, 167.69 μmol, TFA salt) in anhydrous DMF (5 mL) were added EDC·HCl (96.44 mg, 503.06 μmol), DMAP (122.92 mg, 1.01 mmol) and HOBt (45.32 mg, 335.38 μmol) at room temperature. The resulting mixture was stirred at room temperature. After 24 h, the solvent was removed from the reaction mixture under reduced pressure 1.5 N aqueous HCl (80 mL) was added to the residue. The precipitate was filtered and dried to get N-[7-benzyloxy-5-fluoro-6-(1,1,4-trioxo-1,2,5-thiadiazolidin-2-yl)-2-naphthyl]-2-[4-[3-(2,6-dioxo-3-piperidyl)-1-ethyl-indazol-6-yl]-3,3-difluoro-1-piperidyl]acetamide (3, 120 mg, 96.45 μmol, 58% yield) as an off-white solid.

LCMS (ES+): m/z 818.2 [M+H]⁺

### Step 2: 2-[4-[3-(2,6-dioxo-3-piperidyl)-1-ethyl-indazol-6-yl]-3,3-difluoro-1-piperidyl]-N-[5-fluoro-7-hydroxy-6-(1,1,4-trioxo-1,2,5-thiadiazolidin-2-yl)-2-naphthyl]acetamide (Degrader 11)

Into a 50 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[4-[3-(2,6-dioxo-3-piperidyl)-1-ethyl-indazol-6-yl]-3,3-difluoro-1-piperidyl]acetamide (3, 120 mg, 96.84 μmol, crude), pentamethyl benzene (71.78 mg, 484.21 μmol, 78.28 μL) in anhydrous DCM (2.5 mL) and toluene (2.5 mL) was added a 1.0 M solution of boron trichloride in DCM (226.94 mg, 1.94 mmol, 1.94 mL) at −78° C. After completion of the addition reaction mixture was stirred at room temperature. After 3 h, the reaction mixture was quenched with 5% MeOH in DCM (3 mL) at −78° C. The volatiles were removed under reduced pressure and the residue was triturated with MTBE (50 mL), filtered and dried. The crude compound was purified by reverse phase prep HPLC [Purification method: Column: X-Select C18 (250×19) mm, 5 micron; Mobile phase: 0.1% Formic Acid in water; Mobile phase B: MeCN] to get 2-[4-[3-(2,6-dioxo-3-piperidyl)-1-ethyl-indazol-6-yl]-3,3-difluoro-1-piperidyl]-N-[5-fluoro-7-hydroxy-6-(1,1,4-trioxo-1,2,5-thiadiazolidin-2-yl)-2-naphthyl]acetamide (Degrader 11, 33 mg, 42.16 μmol, 44% yield, Formic acid salt) as an off-white solid.

LCMS (ES+): m/z 728.2 [M+H]⁺

¹H NMR (400 MHz, DMSO) δ 10.90 (s, 1H), 10.35 (s, 1H), 10.28 (s, 1H), 8.17 (s, 1H), 7.89 (d, J=9.0 Hz, 1H), 7.67 (d, J=8.4 Hz, 1H), 7.58 (s, 1H), 7.49 (d, J=9.0 Hz, 1H), 7.11 (d, J=8.7 Hz, 1H), 7.00 (s, 1H), 4.46-4.34 (m, 3H), 4.32 (s, 2H), 3.70 (s, 2H), 2.95-2.81 (m, 1H), 2.74-2.57 (m, 3H), 2.44-2.34 (m, 2H), 2.24-2.12 (m, 1H), 2.03-1.91 (m, 1H), 1.38 (t, J=7.2 Hz, 3H).

### 3-[6-[3,3-difluoro-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-1-ethyl-indazol-3-yl]piperidine-2,6-dione (Degrader 12)

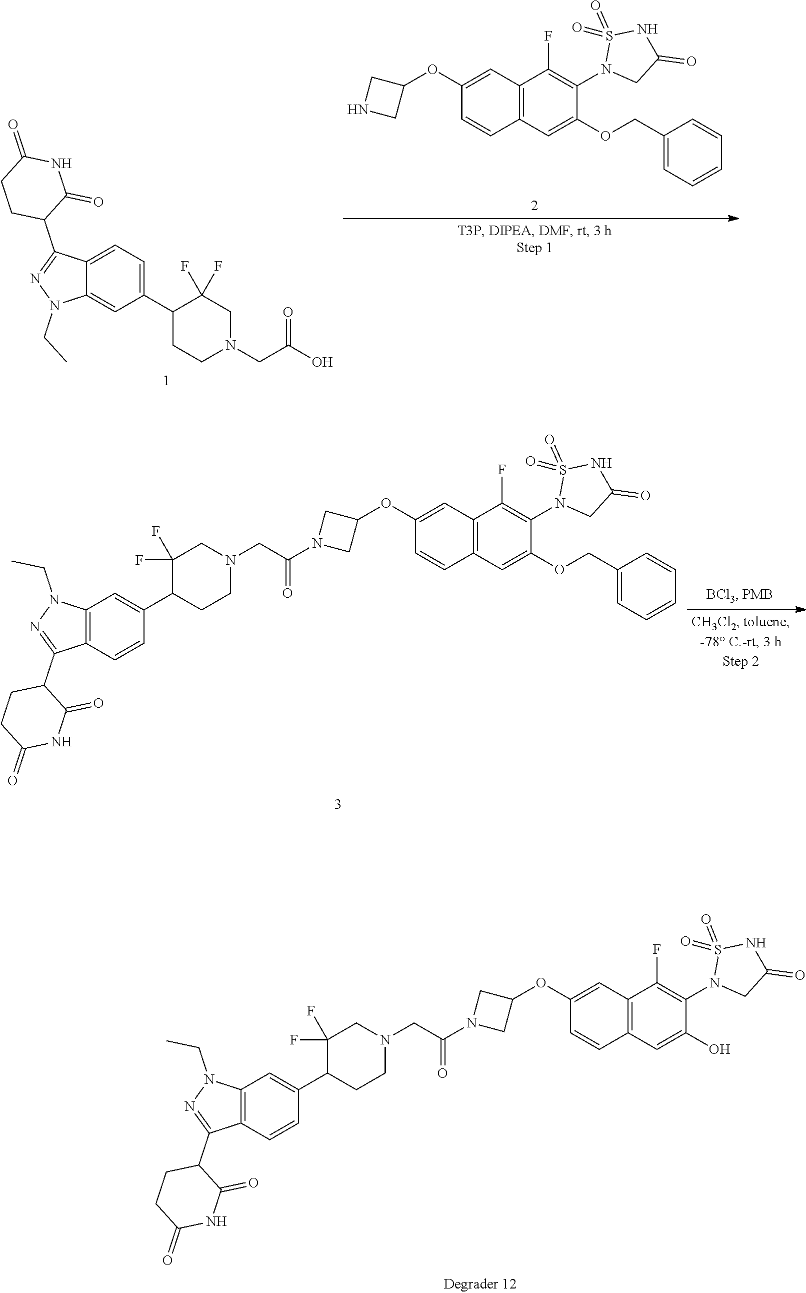 [SMILES: CCn1nc(C2CCC(=O)NC2=O)c2ccc(C3CCN(CC(=O)O)CC3(F)F)cc21 ; O=C1CN(c2c(OCc3ccccc3)cc3ccc(OC4CNC4)cc3c2F)S(=O)(=O)N1 ; CCn1nc(C2CCC(=O)NC2=O)c2ccc(C3CCN(CC(=O)N4CC(Oc5ccc6cc(OCc7ccccc7)c(N7CC(=O)NS7(=O)=O)c(F)c6c5)C4)CC3(F)F)cc21 ; CCn1nc(C2CCC(=O)NC2=O)c2ccc(C3CCN(CC(=O)N4CC(Oc5ccc6cc(O)c(N7CC(=O)NS7(=O)=O)c(F)c6c5)C4)CC3(F)F)cc21]

### Step 1: 3-[6-[1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]-3,3-difluoro-4-piperidyl]-1-ethyl-indazol-3-yl]piperidine-2,6-dione (3)

Into a 50 mL single-neck, round-bottom flask containing a well-stirred solution of 2-[4-[3-(2,6-dioxo-3-piperidyl)-1-ethyl-indazol-6-yl]-3,3-difluoro-1-piperidyl]acetic acid (1, 140 mg, 234.76 μmol, TFA salt) in anhydrous DMF (3 mL) were added propylphosphonic anhydride solution (>50 wt. % in EtOAc, 746.97 mg, 1.17 mmol) and DIPEA (371.00 mg, 2.87 mmol, 0.5 mL) at room temperature and stirred for 10 min. Subsequently, 5-[7-(azetidin-3-yloxy)-3-benzyloxy-1-fluoro-2-naphthyl]-1,1-dioxo-1,2,5-thiadiazolidin-3-one (2, 115.96 mg, 234.76 μmol, HCl salt) in anhydrous DMF (1.5 mL) was added at room temperature and stirring was continued for 3 h. The solvent was removed and water was added to precipitate a solid that was filtered and dried under reduced pressure to get the 3-[6-[1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]-3,3-difluoro-4-piperidyl]-1-ethyl-indazol-3-yl]piperidine-2,6-dione (3, 145 mg, 92.82 μmol, 40% yield) as an off-white solid.

LCMS (ES+): m/z 874.2 [M+H]⁺

### Step 2: 3-[6-[3,3-difluoro-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-1-ethyl-indazol-3-yl]piperidine-2,6-dione (Degrader 12)

Into a 50 mL single-neck, round-bottom flask containing a well-stirred solution of 3-[6-[1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]-3,3-difluoro-4-piperidyl]-1-ethyl-indazol-3-yl]piperidine-2,6-dione (3, 140 mg, 89.71 μmol, crude), pentamethyl benzene (66.50 mg, 448.57 μmol) in anhydrous DCM (3 mL) and toluene (3 mL) was added BCl₃ (1.0 M solution in DCM, 1.79 mmol, 1.79 mL) at −78° C. and the mixture was stirred at room temperature for 3 h. The reaction mixture was quenched with 3 mL of 5% MeOH in DCM at −78° C. Solvents were removed from the reaction mixture under reduced pressure to get the crude compound that was purified by reverse-phase preparative HPLC [Column: X-Bridge C8 (150×19) mm 5 μm; Mobile Phase: 0.1% Formic Acid in Water and MeCN] to get the 3-[6-[3,3-difluoro-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-1-ethyl-indazol-3-yl]piperidine-2,6-dione (Degrader 12, 42 mg, 49.29 μmol, 55% yield, Formic acid salt) as an off-white solid.

LCMS (ES+): m/z 784.2 [M+H]⁺

¹H NMR (400 MHz, DMSO) δ 10.89 (s, 1H), 10.07 (s, 1H), 7.76 (d, J=9.0 Hz, 1H), 7.66 (d, J=8.4 Hz, 1H), 7.55 (s, 1H), 7.23-7.17 (m, 1H), 7.10-6.99 (m, 3H), 5.29-5.20 (m, 1H), 4.75 (q, J=7.3 Hz, 1H), 4.50-4.42 (m, 1H), 4.42-4.33 (m, 4H), 4.30 (s, 2H), 4.28-4.21 (m, 1H), 4.00-3.93 (m, 1H), 3.70-3.62 (m, 2H), 2.92-2.77 (m, 1H), 2.75-2.56 (m, 3H), 2.43-2.35 (m, 2H), 2.21-2.10 (m, 1H), 2.03-1.94 (m, 1H), 1.36 (t, J=7.2 Hz, 3H). Additional protons under solvent peak.

### 3-[6-[3,3-difluoro-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-1-isopropyl-indazol-3-yl]piperidine-2,6-dione (Degrader 13)

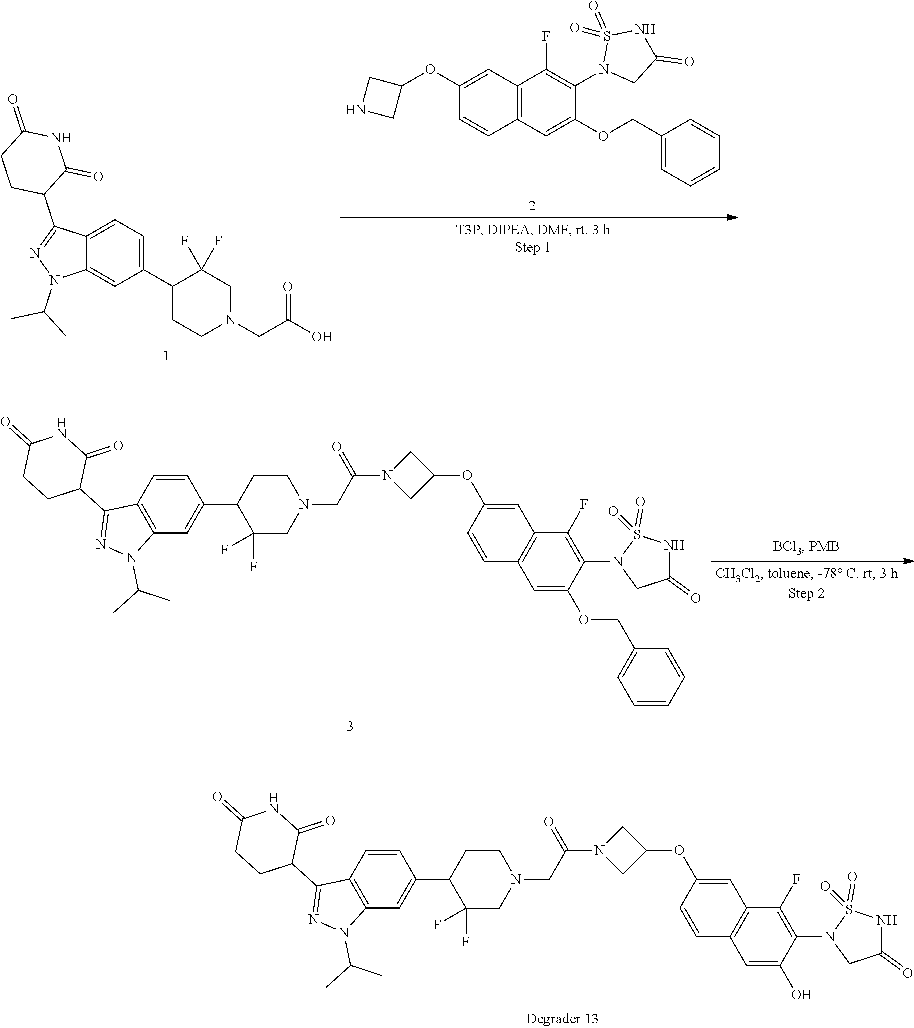 [SMILES: CC(C)n1nc(C2CCC(=O)NC2=O)c2ccc(C3CCN(CC(=O)O)CC3(F)F)cc21 ; O=C1CN(c2c(OCc3ccccc3)cc3ccc(OC4CNC4)cc3c2F)S(=O)(=O)N1 ; CC(C)n1nc(C2CCC(=O)NC2=O)c2ccc(C3CCN(CC(=O)N4CC(Oc5ccc6cc(OCc7ccccc7)c(N7CC(=O)NS7(=O)=O)c(F)c6c5)C4)CC3(F)F)cc21 ; CC(C)n1nc(C2CCC(=O)NC2=O)c2ccc(C3CCN(CC(=O)N4CC(Oc5ccc6cc(O)c(N7CC(=O)NS7(=O)=O)c(F)c6c5)C4)CC3(F)F)cc21]

### Step 1: 3-[6-[1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]-3,3-difluoro-4-piperidyl]-1-isopropyl-indazol-3-yl]piperidine-2,6-dione (3)

Into a 100 mL single-neck round-bottom flask containing well-stirred solution of 2-[4-[3-(2,6-dioxo-3-piperidyl)-1-isopropyl-indazol-6-yl]-3,3-difluoro-1-piperidyl]acetic acid (1, 200 mg, 312.90 μmol, TFA salt) and 5-[7-(azetidin-3-yloxy)-3-benzyloxy-1-fluoro-2-naphthyl]-1,1-dioxo-1,2,5-thiadiazolidin-3-one (2, 192.28 mg, 312.90 μmol, TFA salt) in anhydrous DMF (0.2 mL) were added 1-propanephosphonic anhydride solution (50 wt. % in EtOAc, 597.35 mg, 938.69 μmol) and DIPEA (202.20 mg, 1.56 mmol, 272.51 μL) at ambient temperature under nitrogen atmosphere. The reaction mixture was stirred at ambient temperature for 3 h. The reaction mixture was concentrated under reduced pressure, diluted with cold water (20 mL) to precipitate a solid that was filtered and dried under reduced pressure to afford crude 3-[6-[1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]-3,3-difluoro-4-piperidyl]-1-isopropyl-indazol-3-yl]piperidine-2,6-dione (3, 210 mg, 167.92 μmol, 54% yield) as an off-white solid which was taken as such for the next step.

LCMS (ES+): m/z 888.2 [M+H]⁺

### Step 2: 3-[6-[3,3-difluoro-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-1-isopropyl-indazol-3-yl]piperidine-2,6-dione (Degrader 13)

Into a 50 mL single-neck, round-bottom flask containing a well-stirred solution of 3-[6-[1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]-3,3-difluoro-4-piperidyl]-1-isopropyl-indazol-3-yl]piperidine-2,6-dione (3, 210 mg, 167.92 μmol) and pentamethyl benzene (124.26 mg, 839.6 μmol) in anhydrous DCM (3 mL) and toluene (3 mL) was added BCl₃ (1.0 M solution in DCM, 4.20 mmol, 4.20 mL) at −78° C. After completion of the addition reaction mixture was stirred at room temperature for 3 h. The reaction mixture was quenched with 5 mL of 5% MeOH in DCM at −78° C. and allowed to room temperature. Solvents were removed from the reaction mixture under reduced pressure to get the crude material that was purified by reverse-phase preparative HPLC [Column: X-Bridge C8 (150×19) mm, 5 μm; Mobile Phase A: 0.1% Formic Acid in water and Mobile Phase B: MeCN] to get the 3-[6-[3,3-difluoro-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-1-isopropyl-indazol-3-yl]piperidine-2,6-dione (Degrader 13, 50 mg, 54.12 μmol, 32% yield, TFA salt) as a white solid.

LCMS (ES+): m/z 798.2 [M+H]⁺

¹H NMR (400 MHz, DMSO) δ 10.90 (s, 1H), 10.10 (s, 1H), 7.77 (d, J=9.1 Hz, 1H), 7.65 (d, J=8.2 Hz, 1H), 7.57 (s, 1H), 7.24-7.17 (m, 1H), 7.11-7.04 (m, 2H), 7.02 (s, 1H), 5.31-5.19 (m, 1H), 5.02-4.87 (m, 1H), 4.81-4.65 (m, 1H), 4.52-4.42 (m, 1H), 4.41-4.19 (m, 4H), 4.00-3.92 (m, 1H), 3.30-3.19 (m, 1H), 2.75-2.58 (m, 2H), 2.43-2.35 (m, 2H), 2.21-2.08 (m, 1H), 2.06-1.93 (m, 1H), 1.49-1.40 (m, 6H). Additional protons under solvent peak.

### 3-[5-[1-[[2-[3-[[8-fluoro-6-hydroxy-7-(1,1,4-trioxo-1,2,5-thiadiazolidin-2-yl)-2-naphthyl]oxy]azetidine-1-carbonyl]cyclopropyl]methyl]-4-piperidyl]-3-methyl-2-oxo-benzimidazol-1-yl]piperidine-2,6-dione (Degrader 14)

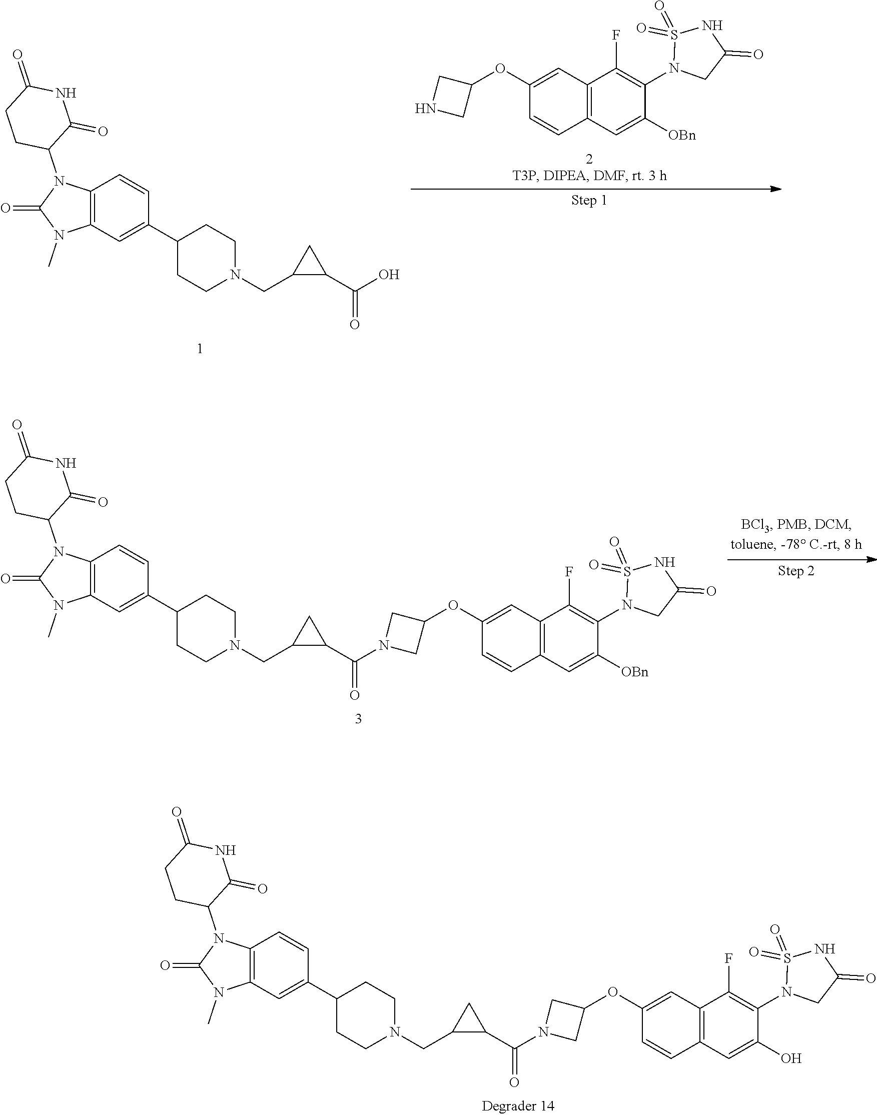 [SMILES: Cn1c(=O)n(C2CCC(=O)NC2=O)c2ccc(C3CCN(CC4CC4C(=O)O)CC3)cc21 ; O=C1CN(c2c(OCc3ccccc3)cc3ccc(OC4CNC4)cc3c2F)S(=O)(=O)N1 ; Cn1c(=O)n(C2CCC(=O)NC2=O)c2ccc(C3CCN(CC4CC4C(=O)N4CC(Oc5ccc6cc(OCc7ccccc7)c(N7CC(=O)NS7(=O)=O)c(F)c6c5)C4)CC3)cc21 ; Cn1c(=O)n(C2CCC(=O)NC2=O)c2ccc(C3CCN(CC4CC4C(=O)N4CC(Oc5ccc6cc(O)c(N7CC(=O)NS7(=O)=O)c(F)c6c5)C4)CC3)cc21]

### Step 1: 3-[5-[1-[[2-[3-[[6-benzyloxy-8-fluoro-7-(1,1,4-trioxo-1,2,5-thiadiazolidin-2-yl)-2-naphthyl]oxy]azetidine-1-carbonyl]cyclopropyl]methyl]-4-piperidyl]-3-methyl-2-oxo-benzimidazol-1-yl]piperidine-2,6-dione (3)

Into a 10 mL single neck round bottom flask containing a well-stirred solution of 2-[[4-[1-(2,6-dioxo-3-piperidyl)-3-methyl-2-oxo-benzimidazol-5-yl]-1-piperidyl]methyl]cyclopropanecarboxylic acid (1, 220.11 mg, 289.77 μmol, TFA salt) in DMF (4 mL) was added DIPEA (187.25 mg, 1.45 mmol) and a 50 wt. % solution of propylphosphonic anhydride in ethyl acetate (368.58 mg, 579.53 μmol, 50% purity) at room temperature. After stirring for 10 min, 5-[7-(azetidin-3-yloxy)-3-benzyloxy-1-fluoro-2-naphthyl]-1,1-dioxo-1,2,5-thiadiazolidin-3-one (2, 180 mg, 289.77 μmol, TFA salt) was added and the reaction mixture was stirred at room temperature. The reaction mixture was concentrated under reduced pressure, the residue was suspended in cold water (7 mL) and triturated. The precipitate was filtered, washed with cold water and dried to afford 3-[5-[1-[[2-[3-[[6-benzyloxy-8-fluoro-7-(1,1,4-trioxo-1,2,5-thiadiazolidin-2-yl)-2-naphthyl]oxy]azetidine-1-carbonyl]cyclopropyl]methyl]-4-piperidyl]-3-methyl-2-oxo-benzimidazol-1-yl]piperidine-2,6-dione (3, 150 mg, 107.73 μmol, 37% yield) as a pale yellow solid.

LCMS (ES+): m/z 881.2 [M+H]⁺

### Step 2: 3-[5-[1-[[2-[3-[[8-fluoro-6-hydroxy-7-(1,1,4-trioxo-1,2,5-thiadiazolidin-2-yl)-2-naphthyl]oxy]azetidine-1-carbonyl]cyclopropyl]methyl]-4-piperidyl]-3-methyl-2-oxo-benzimidazol-1-yl]piperidine-2,6-dione (Degrader 14)

Into a 50 mL single neck round bottom flask containing a well-stirred suspension of 3-[5-[1-[[2-[3-[[6-benzyloxy-8-fluoro-7-(1,1,4-trioxo-1,2,5-thiadiazolidin-2-yl)-2-naphthyl]oxy]azetidine-1-carbonyl]cyclopropyl]methyl]-4-piperidyl]-3-methyl-2-oxo-benzimidazol-1-yl]piperidine-2,6-dione (3, 150 mg, 107.39 μmol) in anhydrous DCM (5 mL) and toluene (5 mL) was added pentamethylbenzene (79.60 mg, 536.96 μmol) at room temperature and cooled to −75° C. Then a 1.0M solution of boron trichloride in dichloromethane (2.5 mmol, 2.15 mL) was added. The reaction mixture was then stirred at room temperature. After 5 h, the reaction mixture was quenched with 5% MeOH in DCM (2 mL) at −75° C. The mixture was concentrated under reduced pressure and the residue was triturated with diethyl ether (10 mL) and filtered. The crude compound was purified by reverse phase prep HPLC [Purification method: Column: X Bridge C18 (150×10) mm, 5 microns; Mobile phase A: 10 mM ammonium bicarbonate in water; Mobile phase B: MeCN] to afford 3-[5-[1-[[2-[3-[[8-fluoro-6-hydroxy-7-(1,1,4-trioxo-1,2,5-thiadiazolidin-2-yl)-2-naphthyl]oxy]azetidine-1-carbonyl]cyclopropyl]methyl]-4-piperidyl]-3-methyl-2-oxo-benzimidazol-1-yl]piperidine-2,6-dione (Degrader 14, 42 mg, 52.29 μmol, 49% yield) as an off-white solid.

LCMS (ES+): m/z 790.2 [M+H]⁺

¹H NMR (400 MHz, DMSO) δ 11.10 (s, 1H), 9.63-9.58 (m, 1H), 7.76-7.69 (m, 1H), 7.19-7.13 (m, 1H), 7.11-6.85 (m, 5H), 5.39-5.30 (m, 1H), 5.28-5.18 (m, 1H), 4.86-4.75 (m, 1H), 4.43-4.33 (m, 1H), 4.29-4.19 (m, 1H), 4.16-4.03 (m, 2H), 3.96-3.85 (m, 1H), 3.64-3.44 (m, 1H), 3.00-2.77 (m, 3H), 2.75-2.57 (m, 3H), 2.11-1.69 (m, 7H), 1.57-1.43 (m, 1H), 1.10-1.04 (m, 1H), 0.98-0.81 (m, 1H). Additional protons under solvent peak.

### 3-[5-[(3R)-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]pyrrolidin-3-yl]-3-methyl-2-oxo-benzimidazol-1-yl]piperidine-2,6-dione (Degrader 15)

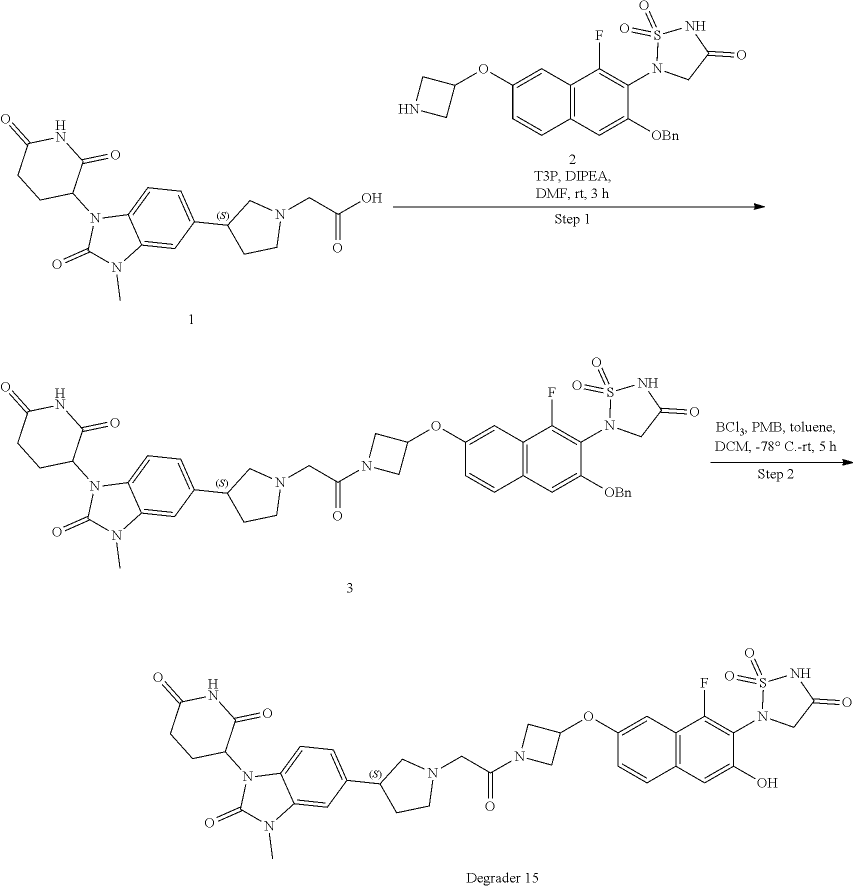

Note: Configurations are arbitrarily assigned.

### Step 1: 3-[5-[(3R)-1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]pyrrolidin-3-yl]-3-methyl-2-oxo-benzimidazol-1-yl]piperidine-2,6-dione (3)

Into a 25 mL single neck round bottom flask containing a well-stirred solution of 2-[(3R)-3-[1-(2,6-dioxo-3-piperidyl)-3-methyl-2-oxo-benzimidazol-5-yl]pyrrolidin-1-yl]acetic acid (1, 109.09 mg, 209.27 μmol, TFA salt) in DMF (3 mL) were added DIPEA (135.23 mg, 1.05 mmol, 182.26 μL) and a 50 wt. % solution of propylphosphonic anhydride in ethyl acetate (266.37 mg, 418.55 μmol, 50% purity) at room temperature. After 10 min, 5-[7-(azetidin-3-yloxy)-3-benzyloxy-1-fluoro-2-naphthyl]-1,1-dioxo-1,2,5-thiadiazolidin-3-one (130 mg, 209.27 μmol, TFA salt) was added and the reaction mixture was stirred at room temperature. After 3 h, the reaction mixture was concentrated under reduced pressure and the residue was triturated with cold water (3 mL). The precipitate was filtered, washed with cold water (10 mL) and diethyl ether (15 mL) to afford 3-[5-[(3R)-1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]pyrrolidin-3-yl]-3-methyl-2-oxo-benzimidazol-1-yl]piperidine-2,6-dione (3, 65 mg, 65.88 μmol, 31% yield) as a pale yellow solid.

LCMS (ES+): m/z 826.4 [M+H]⁺

### Step 2: 3-[5-[(3R)-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]pyrrolidin-3-yl]-3-methyl-2-oxo-benzimidazol-1-yl]piperidine-2,6-dione (Degrader 15)

Into a 50 mL single neck round bottom flask containing a well-stirred suspension of 3-[5-[(3R)-1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]pyrrolidin-3-yl]-3-methyl-2-oxo-benzimidazol-1-yl]piperidine-2,6-dione (3, 65 mg, 65.33 μmol) in anhydrous DCM (2 mL) and toluene (2 mL) was added pentamethylbenzene (48.42 mg, 326.63 μmol) at room temperature and cooled to −75° C. Then a 1.0 M solution of boron trichloride in dichloromethane (1.31 mmol, 1.3 mL) was added and the resulting mixture was stirred at room temperature. After 5 h, the reaction mixture was quenched with 5% MeoH in DCM (2 mL) at −75° C. The volatiles were removed under reduced pressure and the residue was washed with diethyl ether (5 mL). The crude compound was purified by reverse phase prep HPLC [Purification method: Column: X Bridge C18 (150×10) mm, 5 microns; Mobile phase A: 0.1% FA in water; Mobile phase B: MeCN] to afford 3-[5-[(3R)-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]pyrrolidin-3-yl]-3-methyl-2-oxo-benzimidazol-1-yl]piperidine-2,6-dione (Degrader 15, 13 mg, 16.61 μmol, 25% yield) as an off-white solid.

LCMS (ES+): m/z 736.2 [M+H]⁺

¹H NMR (400 MHz, DMSO) δ 11.11 (s, 1H), 10.32 (s, 1H), 9.77 (s, 1H), 7.74 (d, J=9.1 Hz, 1H), 7.22-7.14 (m, 2H), 7.13-7.07 (m, 1H), 7.06 (s, 1H), 7.04-6.96 (m, 2H), 5.35 (dd, J=12.7, 5.2 Hz, 1H), 5.31-5.24 (m, 1H), 4.75-4.68 (m, 1H), 4.56-4.46 (m, 1H), 4.28-4.19 (m, 3H), 4.17-4.07 (m, 2H), 4.05-4.00 (m, 1H), 3.35 (s, 3H), 2.96-2.83 (m, 1H), 2.77-2.60 (m, 3H), 2.21-1.95 (m, 2H). Additional protons under solvent peak.

### 3-[5-[(3S)-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]pyrrolidin-3-yl]-3-methyl-2-oxo-benzimidazol-1-yl]piperidine-2,6-dione (Degrader 16)

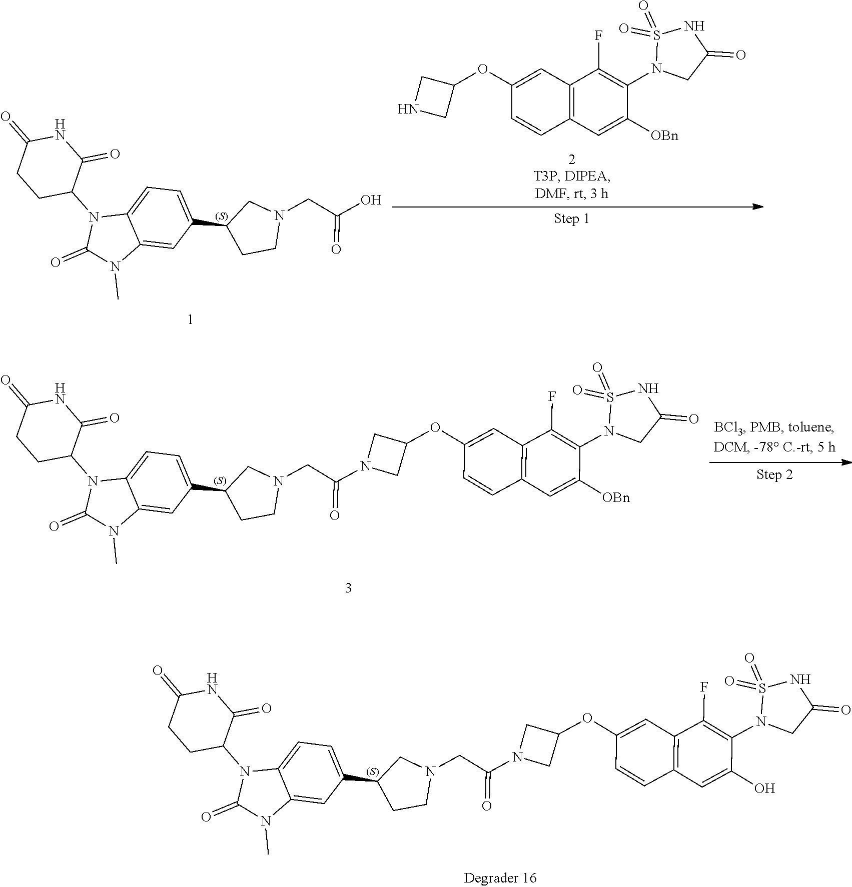

3-[5-[(3S)-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]pyrrolidin-3-yl]-3-methyl-2-oxo-benzimidazol-1-yl]piperidine-2,6-dione (Degrader 16, 10 mg, 12.47 μmol, 25% yield, Formic acid salt) was synthesized in two steps following the same procedure as 3-[5-[(3R)-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]pyrrolidin-3-yl]-3-methyl-2-oxo-benzimidazol-1-yl]piperidine-2,6-dione LCMS (ES+): m/z 736.2 [M+H]⁺

¹HNMR (400 MHz, DMSO-d6): δ 11.11 (s, 1H), 10.29 (s, 1H), 9.69 (s, 1H), 7.74 (d, J=8.80 Hz, 1H), 7.19-6.98 (m, 6H), 5.38-5.28 (m, 2H), 4.72 (t, J=6.40 Hz, 1H), 4.50 (t, J=7.20 Hz, 1H), 4.22-4.00 (m, 7H), 2.93-2.86 (m, 2H), 2.73-2.61 (m, 2H), 2.14-2.00 (m, 2H).

### 3-[5-[1-[[2-[(3R)-3-[8-fluoro-6-hydroxy-7-(1,1,4-trioxo-1,2,5-thiadiazolidin-2-yl)-2-naphthyl]pyrrolidine-1-carbonyl]cyclopropyl]methyl]-4-piperidyl]-3-methyl-2-oxo-benzimidazol-1-yl]piperidine-2,6-dione (Degrader 17)

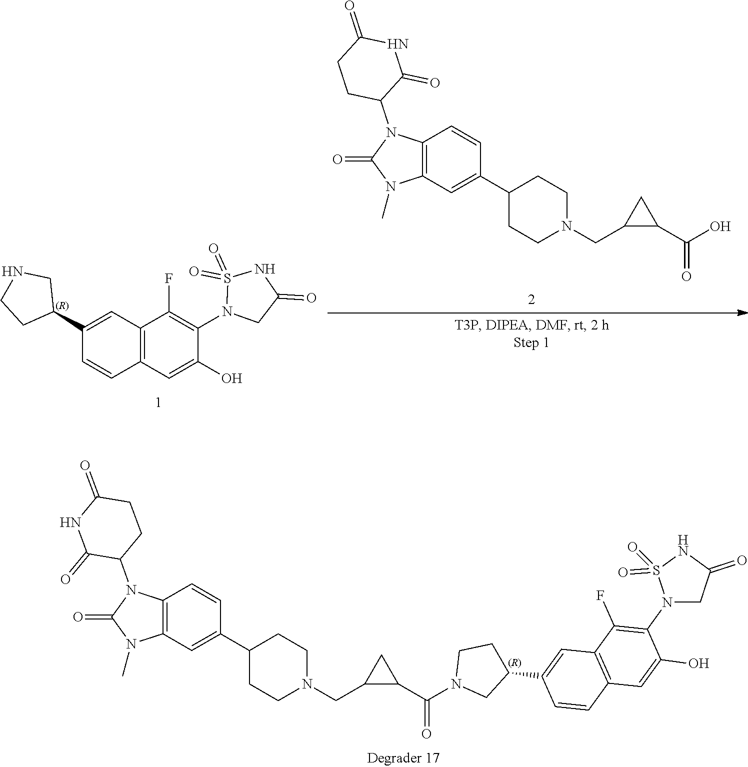

### Step 1: 3-[5-[1-[[2-[(3R)-3-[8-fluoro-6-hydroxy-7-(1,1,4-trioxo-1,2,5-thiadiazolidin-2-yl)-2-naphthyl]pyrrolidin-1-carbonyl]cyclopropyl]methyl]-4-piperidyl]-3-methyl-2-oxo-benzimidazol-1-yl]piperidine-2,6-dione (Degrader 17)

Into a 20 mL screw-capped vial containing a well-stirred solution of 2-[[4-[1-(2,6-dioxo-3-piperidyl)-3-methyl-2-oxo-benzimidazol-5-yl]-1-piperidyl]methyl]cyclopropanecarboxylic acid (2, 120 mg, 162.30 μmol, TFA salt) in DMF (2.5 mL) was added DIPEA (104.88 mg, 811.52 μmol, 141.35 μL) and propylphosphonic anhydride solution (50 wt % in EtOAc, 154.93 mg, 243.46 μmol, 50% purity). The reaction mixture was stirred at room temperature for 5 min. Subsequently, 5-[1-fluoro-3-hydroxy-7-[(3R)-pyrrolidin-3-yl]-2-naphthyl]-1,1-dioxo-1,2,5-thiadiazolidin-3-one (1, 87.34 mg, 178.53 μmol, TFA salt) was added and stirring was continued for 2 h. The reaction mixture was concentrated under vacuum to get crude material that was purified by reverse-phase preparative-HPLC [Column: X-Select C18 (150×19) mm, 5 μm; Mobile phase A: 0.1% TFA in water and Mobile phase B: CH₃CN] to get 3-[5-[1-[[2-[(3R)-3-[8-fluoro-6-hydroxy-7-(1,1,4-trioxo-1,2,5-thiadiazolidin-2-yl)-2-naphthyl]pyrrolidine-1-carbonyl]cyclopropyl]methyl]-4-piperidyl]-3-methyl-2-oxo-benzimidazol-1-yl]piperidine-2,6-dione (Degrader 17 45 mg, 47.41 μmol, 29% yield, TFA Salt) as an off-white solid.

LCMS (ES+): m/z 788.2 [M+H]⁺

¹H-NMR (400 MHz, DMSO-d6): δ 11.11 (s, 1H), 9.95 (brs, 1H), 9.22 (brs, 1H), 7.78-7.69 (m, 2H), 7.53-7.45 (m, 1H), 7.10-6.90 (m, 4H), 5.36 (dd, J=5.20, 12.20 Hz, 1H), 4.17-4.16 (m, 2H), 3.89-3.61 (m, 5H), 3.50-3.25 (m, 6H), 3.12-3.10 (m, 3H), 2.92-2.88 (m, 2H), 2.73-2.65 (m, 3H), 2.09-2.03 (m, 7H), 1.70-1.52 (m, 1H), 1.21-1.10 (m, 1H), 1.00-0.92 (m, 1H).

### 3-[5-[1-[[2-[(3S)-3-[8-fluoro-6-hydroxy-7-(1,1,4-trioxo-1,2,5-thiadiazolidin-2-yl)-2-naphthyl]pyrrolidine-1-carbonyl]cyclopropyl]methyl]-4-piperidyl]-3-methyl-2-oxo-benzimidazol-1-yl]piperidine-2,6-dione (Degrader 18)

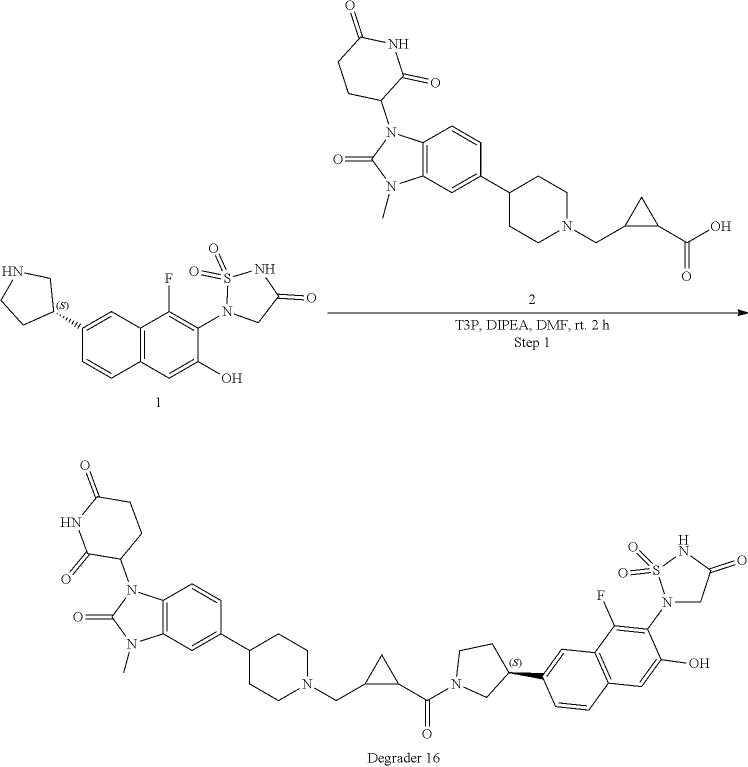

3-[5-[1-[[2-[(3S)-3-[8-fluoro-6-hydroxy-7-(1,1,4-trioxo-1,2,5-thiadiazolidin-2-yl)-2-naphthyl]pyrrolidine-1-carbonyl]cyclopropyl]methyl]-4-piperidyl]-3-methyl-2-oxo-benzimidazol-1-yl]piperidine-2,6-dione (Degrader 18, 50 mg, 54.24 μmol, 33% yield, TFA salt) was synthesized following the same procedure as 3-[5-[1-[[2-[(3R)-3-[8-fluoro-6-hydroxy-7-(1,1,4-trioxo-1,2,5-thiadiazolidin-2-yl)-2-naphthyl]pyrrolidine-1-carbonyl]cyclopropyl]methyl]-4-piperidyl]-3-methyl-2-oxo-benzimidazol-1-yl]piperidine-2,6-dione.

LCMS (ES+): m/z 788.2 [M+H]⁺

¹H-NMR (400 MHz, DMSO-d6): δ 11.11 (s, 1H), 9.84 (brs, 1H), 9.21 (brs, 1H), 7.78-7.69 (m, 2H), 7.51-7.45 (m, 1H), 7.09-6.90 (m, 4H), 5.38-5.34 (m, 1H), 4.21-4.16 (m, 3H), 3.92-3.59 (m, 5H), 3.45-3.25 (m, 5H), 3.12-3.07 (m, 3H), 2.92-2.88 (m, 2H), 2.69-2.61 (m, 2H), 2.61-2.29 (m, 1H), 2.25-1.90 (m, 7H), 1.70-1.52 (m, 1H), 1.21-1.10 (m, 1H), 1.60-0.92 (m, 1H).

### N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)acetamide (Degrader 19)

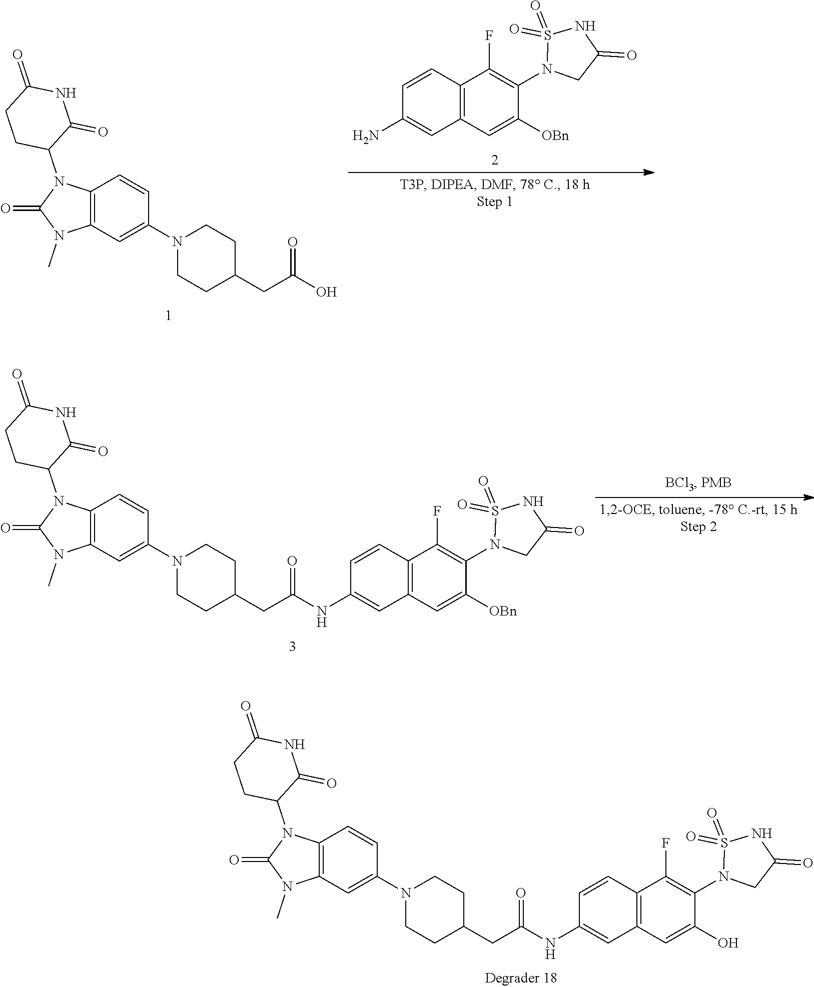 [SMILES: Cn1c(=O)n(C2CCC(=O)NC2=O)c2ccc(N3CCC(CC(=O)O)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(N3CCC(CC(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(N3CCC(CC(=O)Nc4ccc5c(F)c(N6CC(=O)NS6(=O)=O)c(O)cc5c4)CC3)cc21]

### Step 1: N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-2-oxo-benzimidazol-5-yl]-4-piperidyl]acetamide (3)

Into a 50 mL single neck, round bottom flask containing a well-stirred solution of 2-(1-(1-(2,6-dioxopiperidin-3-yl)-3-methyl-2-oxo-2,3-dihydro-1H-benzo[d]imidazol-5-yl)piperidin-4-yl)acetic acid (1, 300 mg, 601.61 μmol) in dry DMF (6 mL) was added propylphosphonic anhydride solution (50 wt % in EtOAc) (765.68 mg, 1.20 mmol) followed by DIPEA (388.76 mg, 3.01 mmol, 523.93 μL). The reaction mixture was stirred at room temperature for 1 h, then 5-(6-amino-3-benzyloxy-1-fluoro-2-naphthyl)-1,1-dioxo-1,2,5-thiadiazolidin-3-one (2, 316.74 mg, 601.61 μmol, TFA salt) was added and the reaction mixture was stirred for 15 h at 70° C. The reaction mixture was concentrated and the residue was purified by reverse-phase preparative HPLC [Column: X-Select C18 (150×19), 5 μm; Mobile Phase A: 0.1% TFA in water and Mobile Phase B: CH₃CN] to afford N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-2-oxo-benzimidazol-5-yl]-4-piperidyl]acetamide (3, 120 mg, 20% yield, TFA salt) as a brown solid. LCMS (ES+): m/z 784.3 [M+H]⁺

### Step 2: N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)acetamide (Degrader 19)

Into a 25 mL single neck round bottom flask containing a well-stirred solution of N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-2-oxo-benzimidazol-5-yl]-4-piperidyl]acetamide (3, 110 mg, 111.73 μmol, TFA salt) in anhydrous DCE (4 mL) and toluene (4 mL) was added pentamethylbenzene (82.82 mg, 558.67 μmol). The reaction mixture was cooled to −78° C. and BCl₃ (1.0 M solution in CH₂Cl₂) (327.30 mg, 2.79 mmol, 2.79 mL) was added. The reaction mixture was stirred at room temperature for 16 h. The reaction was quenched with 5% MeOH in DCM (5 mL) at −78° C. The volatiles were removed and residue triturated with diethyl ether (10 mL) and purified by reverse-phase preparative HPLC [Column: X-Select C18 (150×19), 5 μm; Mobile Phase A: 0.1% TFA in water and Mobile Phase B: CH₃CN] to afford N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)acetamide (Degrader 19 41 mg, 45% yield, TFA salt) as an off-white solid. LCMS (ES+): m/z 694.0 [M+H]⁺

¹H NMR (400 MHz, DMSO-d₆) δ 11.12 (s, 1H), 10.21 (s, 1H), 10.05 (s, 1H), 8.19-8.15 (m, 1H), 7.85 (d, J=8.9 Hz, 1H), 7.44 (dd, J=9.0, 2.0 Hz, 1H), 7.22-7.08 (m, 2H), 6.95 (s, 1H), 5.38 (dd, J=12.9, 5.4 Hz, 1H), 4.23 (s, 2H), 3.62 (d, J=11.3 Hz, 2H), 3.36 (s, 3H), 2.90 (ddd, J=16.9, 12.7, 5.2 Hz, 1H), 2.78-2.58 (m, 2H), 2.43 (d, J=6.8 Hz, 2H), 2.23-2.08 (m, 1H), 2.05-1.88 (m, 3H), 1.71-1.53 (m, 2H).

### N-[5-fluoro-7-hydroxy-6-(1,1,4-trioxo-1,2,5-thiadiazolidin-2-yl)-2-naphthyl]-2-[1-[3-methyl-2-oxo-1-[(3S)-2,6-dioxo-3-piperidyl]benzimidazol-5-yl]-4-piperidyl]acetamide (Degrader 20) and N-[5-fluoro-7-hydroxy-6-(1,1,4-trioxo-1,2,5-thiadiazolidin-2-yl)-2-naphthyl]-2-[1-[3-methyl-2-oxo-1-[(3R)-2,6-dioxo-3-piperidyl]benzimidazol-5-yl]-4-piperidyl]acetamide (Degrader 21)

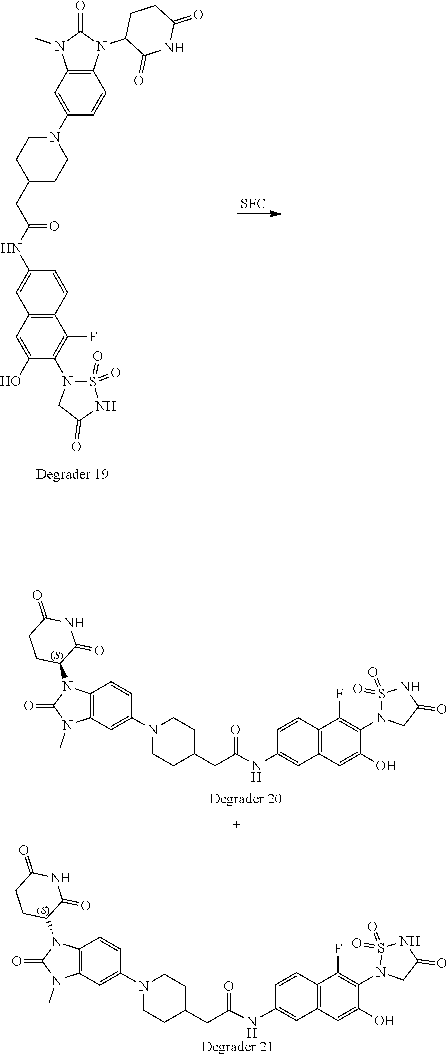

Note: Configurations are arbitrarily assigned.

2-[1-[1-(2,6-dioxo-3-piperidyl)-3-methyl-2-oxo-benzimidazol-5-yl]-4-piperidyl]-N-[5-fluoro-7-hydroxy-6-(1,1,4-trioxo-1,2,5-thiadiazolidin-2-yl)-2-naphthyl]acetamide (Degrader 19, 530 mg, 0.716 mmol, Formic acid salt) was subjected to chiral SFC purification. Method details: Column Name: (R,R) Whelk-01, Flow rate: 5 ml/min, Co-Solvent: 45%, Co-Solvent Name: 0.1% IPAm in IPA:ACN (1:1), Injected Volume: 15 μL, Temperature: 35° C., Outlet Pressure: 100 bar.

Chiral SFC: Method of Analysis Instrument: PIC 175; Column: (R, R) Whelk-01 (250×30) mm, 5 μm; Mobile Phase: CO2: 0.1% Isopropylamine in Isopropyl alcohol: Acetonitrile (1:1): (50:50); Total Flow: 90 g/min; Back pressure: 100 bar; Wavelength: 254 nmu; Cycle time: 10 min; 100 mg was dissolved in 2.5 mL of ACN as well as in Mobile phase and injected 700 L/injection.

After SFC purification, the first eluted fraction at RT 5.5 min was acidified with TFA and concentrated and purified by reverse-phase preparative HPLC [X-Bridge OBD C18 (19×150) mm, 5 M column, Mobile phase A: 0.1% TFA in water and Mobile Phase B: MeCN] to afford N-[5-fluoro-7-hydroxy-6-(1,1,4-trioxo-1,2,5-thiadiazolidin-2-yl)-2-naphthyl]-2-[1-[3-methyl-2-oxo-1-[(3S)-2,6-dioxo-3-piperidyl]benzimidazol-5-yl]-4-piperidyl]acetamide (Degrader 20, 75 mg, 90.08 μmol, 9% yield, TFA salt) as a white solid.

LCMS (ES+): m/z 694.2 [M+H]⁺

¹HNMR (400 MHz, DMSO-d6): δ 11.14 (s, 1H), 10.24 (s, 1H), 10.14 (s, 1H), 8.19 (s, 1H), 7.86 (d, J=8.80 Hz, 1H), 7.45 (dd, J=1.60, 8.80 Hz, 1H), 7.25-7.11 (m, 2H), 6.96 (s, 1H), 5.41-5.38 (m, 1H), 4.26 (s, 2H), 3.64-3.61 (m, 2H), 3.37 (s, 3H), 2.94-2.87 (m, 2H), 2.77-2.61 (m, 2H), 2.50-2.43 (m, 2H), 2.23-2.12 (m, 1H), 2.04-2.01 (m, 3H), 1.71-1.54 (m, 2H).

Note: One of the aromatic protons was not resonated. Appears in D₂O exchange data.

Similarly, the second eluted fraction at RT 6.6 min was acidified with TFA and concentrated and purified by reverse-phase preparative HPLC [X-Bridge OBD C18 (19×150) mm, 5 m column, Mobile phase A: 0.1% TFA in water and Mobile Phase B: MeCN] to afford N-[5-fluoro-7-hydroxy-6-(1,1,4-trioxo-1,2,5-thiadiazolidin-2-yl)-2-naphthyl]-2-[1-[3-methyl-2-oxo-1-[(3R)-2,6-dioxo-3-piperidyl]benzimidazol-5-yl]-4-piperidyl]acetamide (Degrader 21, 70 mg, 79.87 μmol, 8% yield, TFA salt) as white solid.

LCMS (ES−): m/z 692.1 [M−H]⁻

¹HNMR (400 MHz, DMSO-d6): δ 11.14 (s, 1H), 10.24 (s, 1H), 10.11 (s, 1H), 8.19 (s, 1H), 7.86 (d, J=8.80 Hz, 1H), 7.45 (d, J=7.60 Hz, 1H), 7.22-7.09 (m, 2H), 6.96 (s, 1H), 5.40-5.37 (m, 1H), 4.25 (s, 2H), 3.90-3.88 (m, 2H), 3.37 (s, 3H), 2.94-2.87 (m, 1H), 2.76-2.66 (m, 2H), 2.45-2.43 (m, 2H), 2.19-2.16 (m, 1H), 2.09-2.00 (m, 3H), 1.19-1.14 (m, 2H).

Note: One of the aromatic protons was not resonated. Appears in D₂O exchange data. Some aliphatic protons are merged into solvent peaks.

### N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-ethyl-2-oxo-2,3-dihydro-1H-benzo[d]imidazol-5-yl)piperidin-4-yl)acetamide (Degrader 22)

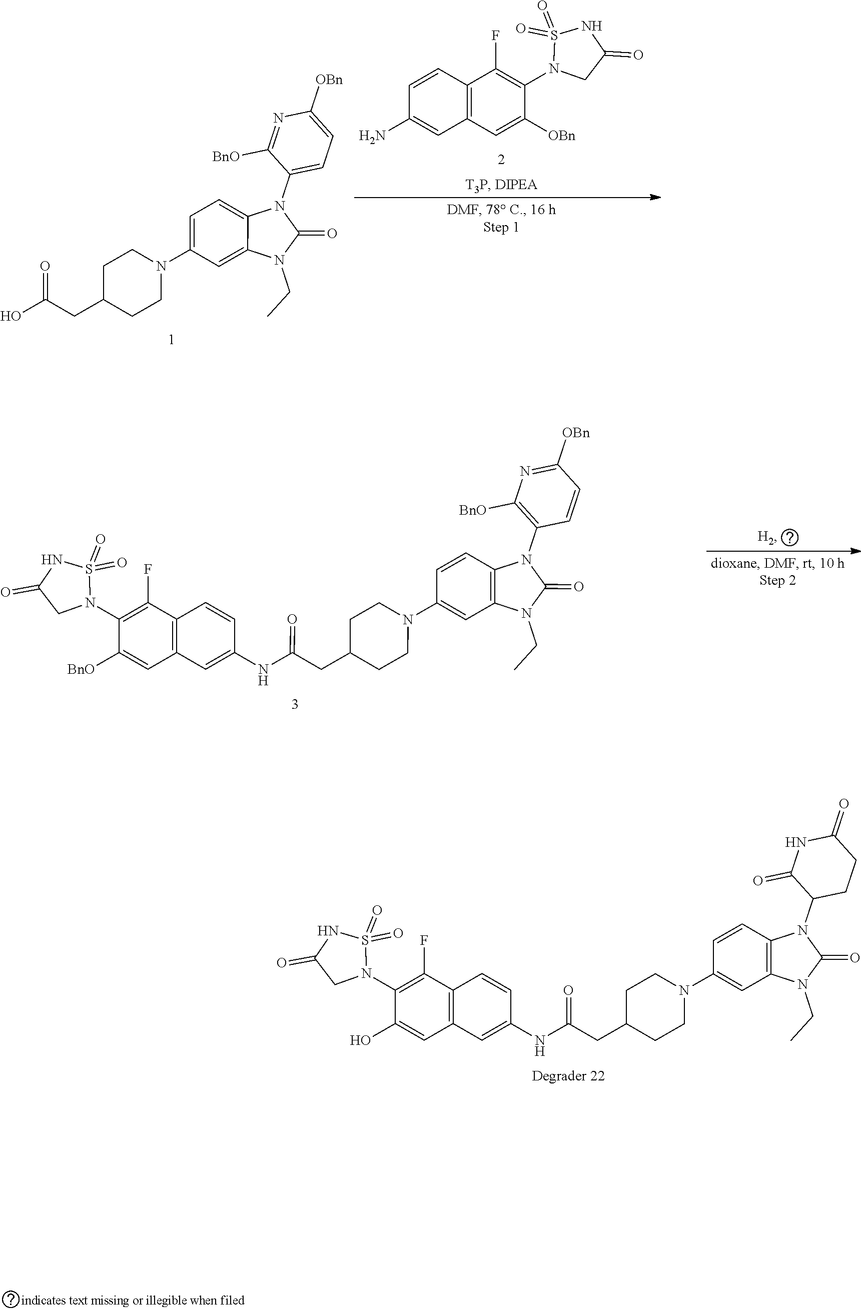 [SMILES: CCn1c(=O)n(-c2ccc(OCc3ccccc3)nc2OCc2ccccc2)c2ccc(N3CCC(CC(=O)O)CC3)cc21 ; Nc1ccc2c(F)c(N3CC(=O)NS3(=O)=O)c(OCc3ccccc3)cc2c1 ; CCn1c(=O)n(-c2ccc(OCc3ccccc3)nc2OCc2ccccc2)c2ccc(N3CCC(CC(=O)Nc4ccc5c(F)c(N6CC(=O)NS6(=O)=O)c(OCc6ccccc6)cc5c4)CC3)cc21 ; CCn1c(=O)n(C2CCC(=O)NC2=O)c2ccc(N3CCC(CC(=O)Nc4ccc5c(F)c(N6CC(=O)NS6(=O)=O)c(O)cc5c4)CC3)cc21]

### Step 1: N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-ethyl-2-oxo-2,3-dihydro-1H-benzo[d]imidazol-5-yl)piperidin-4-yl)acetamide (3)

To a solution of 2-(1-(1-(2,6-bis(benzyloxy)pyridin-3-yl)-3-ethyl-2-oxo-2,3-dihydro-1H-benzo[d]imidazol-5-yl)piperidin-4-yl)acetic acid (1, 100 mg, 168.72 μmol, 1 eq), 5-(6-amino-3-benzyloxy-1-fluoro-2-naphthyl)-1,1-dioxo-1,2,5-thiadiazolidin-3-one (2, 86.97 mg, 168.72 μmol, 1 eq) in DMF (1 mL) were added DIPEA (327.10 mg, 2.53 mmol, 440.83 μL, 15 eq) and T₃P (751.59 mg, 1.18 mmol, 50% purity in EtOAc, 7 eq). The mixture was stirred at 50° C. for 16 h. The reaction mixture was filtered and concentrated under reduced pressure to give a residue. The residue was purified by prep-HPLC (flow: 25 mL/min; gradient: from 76-46% water (0.1% TFA)-ACN; column: Phenomenex Luna C18 75×30 mm×3 μm) to afford N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-ethyl-2-oxo-2,3-dihydro-1H-benzo[d]imidazol-5-yl)piperidin-4-yl)acetamide (3, 80 mg, 82.99 μmol, 49% yield) as a white solid.

LCMS (ESI): m/z 977.2 [M+H]⁺

### Step 2: N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-ethyl-2-oxo-2,3-dihydro-1H-benzo[d]imidazol-5-yl)piperidin-4-yl)acetamide (Degrader 22)

To a solution of N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-ethyl-2-oxo-2,3-dihydro-1H-benzo[d]imidazol-5-yl)piperidin-4-yl)acetamide (3, 80 mg, 81.96 μmol, 1 eq) in dioxane (1 mL), DMF (1 mL) was added Pd/C (20 mg, 10% purity) and Pd(OH)₂/C (20 mg, 10% purity). The mixture was stirred at 20° C. for 16 h under H₂ (15 Psi). The reaction mixture was filtered and concentrated under reduced pressure to give a residue. The residue was purified by prep-HPLC (flow: 25 mL/min; gradient: from 39-9% water (0.1% TFA)-ACN; column: Phenomenex Luna C18 75×30 mm×3 μm) to afford N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-ethyl-2-oxo-2,3-dihydro-1H-benzo[d]imidazol-5-yl)piperidin-4-yl)acetamide (Degrader 22, 30 mg, 34.68 μmol, 42% yield) as an off-white solid.

LCMS (ESI): m/z 708.2 [M+H]⁺

¹H NMR (400 MHz, DMSO-d₆) δ 11.11 (s, 1H), 10.32-9.90 (m, 2H), 8.17 (s, 1H), 7.85 (d, J=9.2 Hz, 1H), 7.54-7.28 (m, 2H), 7.25-7.04 (m, 2H), 7.00-6.92 (m, 1H), 5.44-5.31 (m, 1H), 4.21 (br s, 2H), 3.91-3.86 (m, 2H), 3.61 (br d, J=9.3 Hz, 4H), 3.01-2.84 (m, 2H), 2.73-2.63 (m, 2H), 2.22-2.12 (m, 1H), 2.08-1.90 (m, 4H), 1.71-1.56 (m, 2H), 1.24 (br t, J=7.1 Hz, 3H).

### 2-[(3S)-4-[1-(2,6-dioxo-3-piperidyl)-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 23)

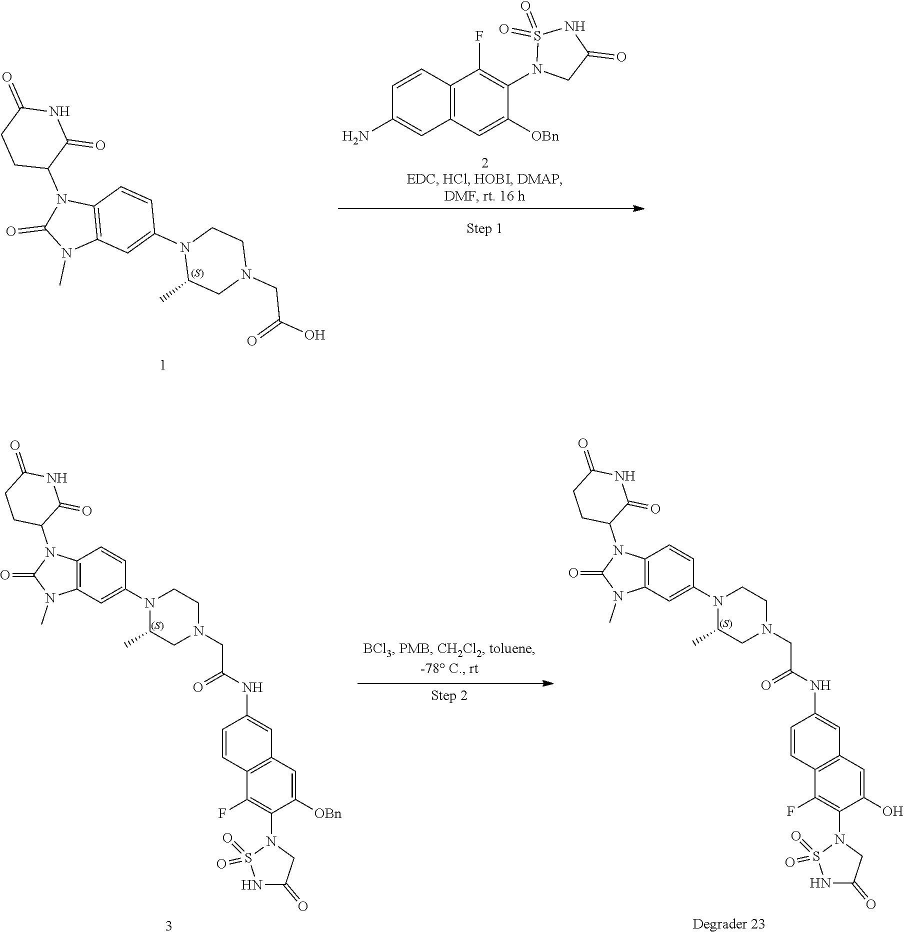

### Step 1: N-[7-benzyloxy-5-fluoro-6-(1,1,4-trioxo-1,2,5-thiadiazolidin-2-yl)-2-naphthyl]-2-[(3S)-4-[1-(2,6-dioxo-3-piperidyl)-3-methyl-2-oxo-benzimidazol-5-yl]-3-methyl-piperazin-1-yl]acetamide (4)

Into a 50 mL single-neck round-bottom flask containing a well-stirred solution of 2-[(3S)-4-[1-(2,6-dioxo-3-piperidyl)-3-methyl-2-oxo-benzimidazol-5-yl]-3-methyl-piperazin-1-yl]acetic acid (1, 93.06 mg, 219.53 μmol) and 5-(6-amino-3-benzyloxy-1-fluoro-2-naphthyl)-1,1-dioxo-1,2,5-thiadiazolidin-3-one (2, 117.49 mg, 263.43 μmol) in anhydrous DMF (10 mL) were added EDC·HCl (126.25 mg, 658.57 μmol), HOBT (59.32 mg, 439.05 μmol) and DMAP (134.10 mg, 1.10 mmol). After 16 h, the reaction mixture was concentrated under reduced pressure. The reside was treated with 1.5 N aqueous HCl (20 mL) and the precipitated solid was filtered and dried under reduced pressure to get N-[7-benzyloxy-5-fluoro-6-(1,1,4-trioxo-1,2,5-thiadiazolidin-2-yl)-2-naphthyl]-2-[(3 S)-4-[1-(2,6-dioxo-3-piperidyl)-3-methyl-2-oxo-benzimidazol-5-yl]-3-methyl-piperazin-1-yl]acetamide (3, 180 mg, 128.44 μmol, 59% yield) as an off-white solid. The crude compound was used in the next step.

LCMS (ES+): m/z 799.20 [M+H]⁺

### Step 2: 2-[(3S)-4-[1-(2,6-dioxo-3-piperidyl)-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 23)

Into a 50 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[(3S)-4-[1-(2,6-dioxo-3-piperidyl)-3-methyl-2-oxo-benzimidazol-5-yl]-3-methyl-piperazin-1-yl]acetamide (3, 160 mg, 114.17 μmol) in a mixture of anhydrous DCM (5 mL) and toluene (5 mL) was added pentamethylbenzene (84.63 mg, 570.83 μmol). The mixture was cooled to −78° C. and added a 1.0 M solution of boron trichloride in DCM (2.28 mmol, 2.28 mL). The reaction mixture was stirred at ambient temperature. After 5 h the reaction mixture was cooled to −78° C. and diluted with DCM (5 mL), The mixture was concentrated under reduced pressure and the residue was triturated with MTBE (5 mL), filtered and dried. The crude compound was purified by reverse phase preparatory HPLC [Purification method: Column: Xbridge C18 (20×150) mm; Mobile phase: A: 0.1% Formic acid in water, B: Acetonitrile] to obtain 2-[(3S)-4-[1-(2,6-dioxo-3-piperidyl)-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 23, 38 mg, 50.01 μmol, 44% yield, TFA salt) as a colorless solid.

LCMS (ES+): m/z 709.0 [M+H]⁺

¹HNMR (400 MHz, DMSO-d6): δ 11.10 (s, 1H), 10.83 (bs, 1H), 9.86 (s, 1H), 8.14 (s, 1H), 7.90 (d, J=8.80 Hz, 1H), 7.48 (d, J=8.80 Hz, 1H), 7.22-6.66 (m, 4H), 5.41-5.30 (m, 1H), 4.40-4.14 (m, 1H), 4.09 (s, 2H), 3.71-3.55 (m, 2H), 3.36-3.12 (m, 7H), 2.94-2.87 (m, 2H), 2.75-2.61 (m, 3H), 2.03-2.01 (m, 1H), 1.10-0.92 (m, 3H).

### 2-[(3R)-4-[1-(2,6-dioxo-3-piperidyl)-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 24)

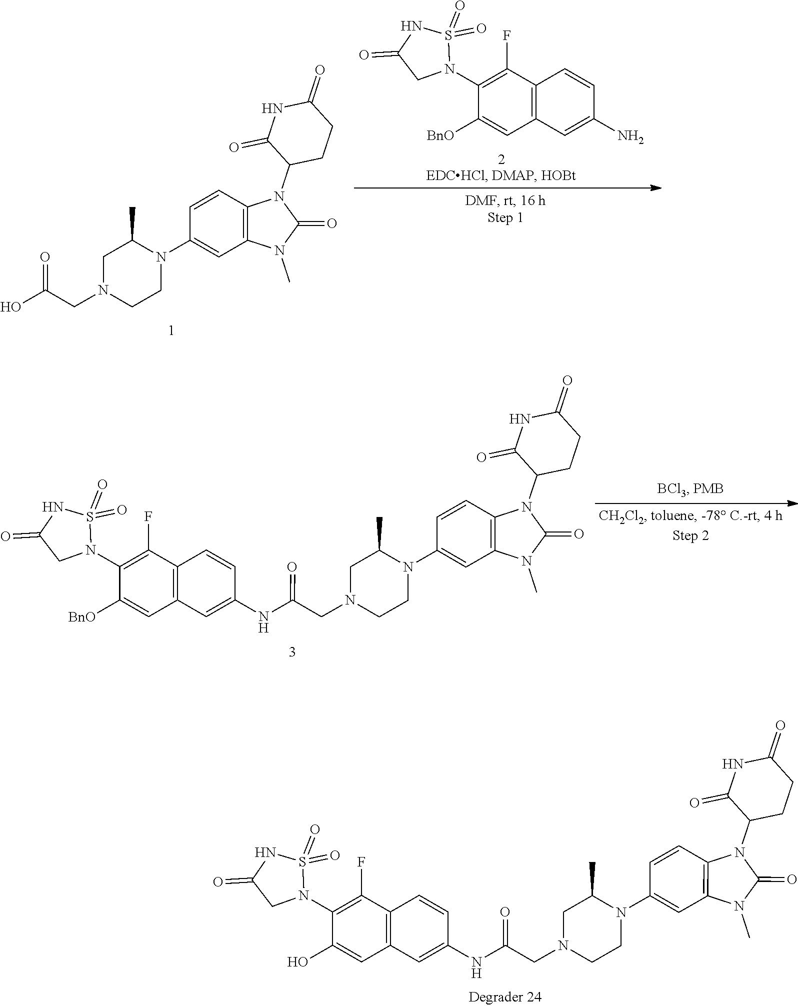

### Step 1: N-[7-benzyloxy-5-fluoro-6-(1,1,4-trioxo-1,2,5-thiadiazolidin-2-yl)-2-naphthyl]-2-[(3R)-4-[1-(2,6-dioxo-3-piperidyl)-3-methyl-2-oxo-benzimidazol-5-yl]-3-methyl-piperazin-1-yl]acetamide (3)

Into a 20 mL screw-capped vial containing a well-stirred solution of 2-[(3R)-4-[1-(2,6-dioxo-3-piperidyl)-3-methyl-2-oxo-benzimidazol-5-yl]-3-methyl-piperazin-1-yl]acetic acid (1, 205.44 mg, 368.62 μmol, TFA salt) and 5-(6-amino-3-benzyloxy-1-fluoro-2-naphthyl)-1,1-dioxo-1,2,5-thiadiazolidin-3-one (2, 200 mg, 368.62 μmol, TFA salt) in DMF (5 mL) were added sequentially EDC·HCl (212.00 mg, 1.11 mmol), DMAP (225.17 mg, 1.84 mmol) and HOBt (99.62 mg, 737.24 μmol) at room temperature. The resulting mixture was stirred at room temperature for 16 h. The reaction mixture concentrated under reduced pressure and diluted with cold water (50 mL) to obtain a solid that was filtered, washed with 1.5 N HCl, water and dried over reduced pressure to afford N-[7-benzyloxy-5-fluoro-6-(1,1,4-trioxo-1,2,5-thiadiazolidin-2-yl)-2-naphthyl]-2-[(3R)-4-[1-(2,6-dioxo-3-piperidyl)-3-methyl-2-oxo-benzimidazol-5-yl]-3-methyl-piperazin-1-yl]acetamide (3, 190 mg, 107.24 μmol, 29% yield) as a brown solid.

LCMS (ES+): m/z 799.2 [M+H]⁺

### Step 2: 2-[(3R)-4-[1-(2,6-dioxo-3-piperidyl)-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 24)

Into a 25 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[(3R)-4-[1-(2,6-dioxo-3-piperidyl)-3-methyl-2-oxo-benzimidazol-5-yl]-3-methyl-piperazin-1-yl]acetamide (3, 200 mg, 112.84 μmol) in anhydrous toluene (3 mL) and DCM (3 mL) was added pentamethylbenzene (83.64 mg, 564.19 μmol, 91.21 μL) at room temperature. The resulting mixture was cooled to −78° C. and added BCl₃ (1.0 M solution in DCM, 4.0 mmol, 4.0 mL) dropwise. The resultant reaction mixture was stirred at room temperature for 4 h. The reaction mixture was cooled to −78° C. and quenched with 1% DCM in MeOH (10 mL). The solvent was evaporated under reduced pressure to obtain the crude material that was purified by reverse-phase preparative HPLC [Column: X SELECT C18 (150×19) mm, 5 μm; Mobile Phase A: 0.1% TFA in Water and Mobile Phase B: CH₃CN] to afford 2-[(3R)-4-[1-(2,6-dioxo-3-piperidyl)-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 24, 35.15 mg, 41.33 μmol, 37% yield, TFA salt) as an off-white solid.

LCMS (ES+): m/z 709.0 [M+H]⁺

¹H-NMR (400 MHz, DMSO-d6): δ 11.11 (s, 1H), 10.82 (s, 1H), 10.03 (s, 1H), 8.14 (s, 1H), 7.92 (d, J=9.20 Hz, 1H), 7.49 (d, J=9.20 Hz, 1H), 7.09-6.91 (m, 3H), 6.69-6.68 (m, 1H), 5.38 (dd, J=60.00, Hz, 1H), 4.32 (s, 2H), 4.18 (s, 2H), 3.58-3.47 (m, 3H), 3.32 (s, 3H), 3.07-2.61 (m, 7H), 2.04-2.01 (m, 1H), 0.99-0.89 (m, 3H).

### 2-[4-[1-(2,6-dioxo-3-piperidyl)-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 25)

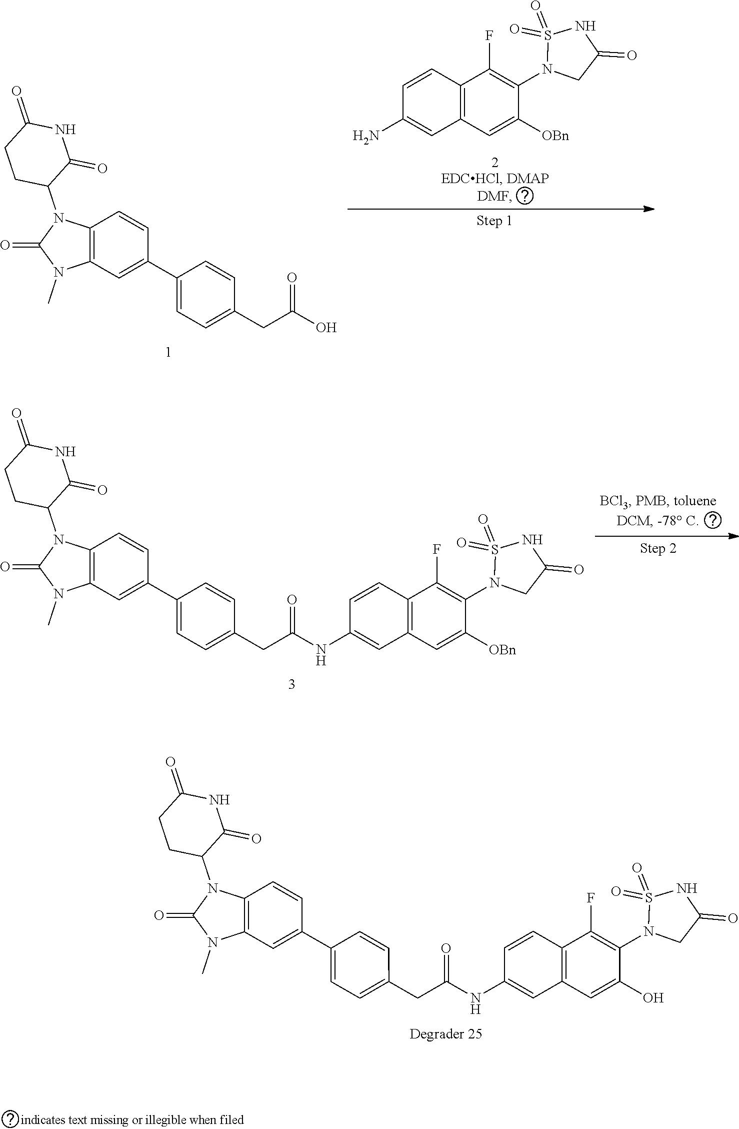 [SMILES: Cn1c(=O)n(C2CCC(=O)NC2=O)c2ccc(-c3ccc(CC(=O)O)cc3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(-c3ccc(CC(=O)Nc4ccc5c(F)c(N6CC(=O)NS6(=O)=O)c(OCc6ccccc6)cc5c4)cc3)cc21 ; Cn1c(=O)n(C2CCC(=O)NC2=O)c2ccc(-c3ccc(CC(=O)Nc4ccc5c(F)c(N6CC(=O)NS6(=O)=O)c(O)cc5c4)cc3)cc21]

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]phenyl]acetamide (3)

To a 25 mL single neck round bottom flask containing a well-stirred solution of 2-[4-[1-(2,6-dioxo-3-piperidyl)-3-methyl-2-oxo-benzimidazol-5-yl]phenyl]acetic acid (1, 230 mg, 555.43 μmol) in DMF (2 mL) were added 4-dimethylaminopyridine (339.28 mg, 2.78 mmol), N-(3-dimethylaminopropyl)-N′-ethylcarbodiimide hydrochloride (159.71 mg, 833.14 μmol). After 2 h a solution of 5-(6-amino-3-benzyloxy-1-fluoro-2-naphthyl)-1,1-dioxo-1,2,5-thiadiazolidin-3-one (2, 301.67 mg, 555.43 μmol, TFA salt) in DMF (1.5 mL) was added. After 16 h, additional quantities of 4-dimethylaminopyridine (339.28 mg, 2.78 mmol) and N-(3-dimethylaminopropyl)-N′-ethylcarbodiimide hydrochloride (159.71 mg, 833.14 μmol) were added. The mixture was stirred at 60° C. for 8 h. The reaction was quenched with ice-water and the precipitate was filtered. The material was purified by reverse phase column chromatography [Purification method: Siliasep C18 120 g, column; Mobile phase A: 0.1% TFA in water; Mobile phase B: MeCN] to afford N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]phenyl]acetamide (3, 170 mg, 194.78 μmol, 35% yield) as a yellow solid. LCMS (ES+): m/z 777.2 [M+H]⁺

### Step 2: 2-[4-[1-(2,6-dioxo-3-piperidyl)-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 25)

To a 25 mL single neck round bottom flask containing a well-stirred suspension of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]phenyl]acetamide (3, 170 mg, 194.78 μmol) in a mixture of toluene (3 mL) and DCM (3 mL), was added pentamethylbenzene (144.37 mg, 973.88 μmol, 157.44 μL) under nitrogen atmosphere. The resulting suspension was cooled to −78° C. and boron trichloride solution (LOM in methylene chloride) (456.44 mg, 3.90 mmol, 3.9 mL) was added drop-wise. The reaction mixture was stirred at ambient temperature. The reaction mixture was cooled to −78° C. and quenched with 5% MeOH in DCM (4 mL) and concentrated under reduced pressure. The residue was triturated with diethyl ether (2×25 mL) and filtered. The material was purified by reverse phase prep HPLC [Purification method: Column: X-Bridge, C18 (19×150 mm), 5 Micron; Mobile phase A: 0.1% TFA in water; Mobile phase B: MeCN] to afford 2-[4-[1-(2,6-dioxo-3-piperidyl)-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 25, 52 mg, 73.87 μmol, 38% yield) as a pale brown solid. LCMS (ES−): m/z 684.8 [M−H]⁻

¹H NMR (400 MHz, DMSO-d₆) δ 11.12 (s, 1H), 10.45 (s, 1H), 8.18 (d, J=1.9 Hz, 1H), 7.87 (d, J=9.0 Hz, 1H), 7.70-7.62 (m, 2H), 7.53-7.41 (m, 4H), 7.35 (dd, J=8.3, 1.7 Hz, 1H), 7.19 (d, J=8.5 Hz, 1H), 6.97-6.94 (m, 1H), 5.40 (dd, J=12.9, 5.4 Hz, 1H), 4.40 (s, 2H), 3.74 (s, 2H), 3.41 (s, 3H), 2.98-2.85 (m, 1H), 2.81-2.70 (m, 1H), 2.69-2.59 (m, 1H), 2.10-1.99 (m, 1H).

### 2-[4-[1-[(3R)-2,6-dioxo-3-piperidyl]-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 26) and 2-[4-[1-[(3S)-2,6-dioxo-3-piperidyl]-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 27)

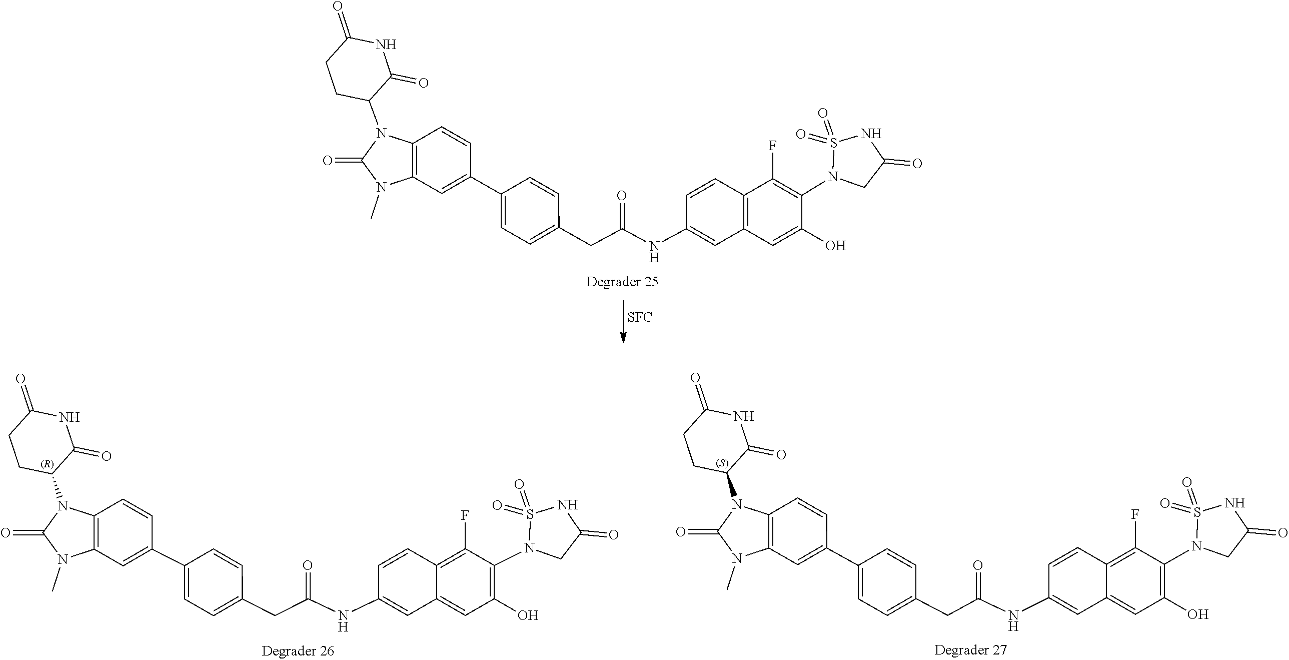

Note: Configurations are arbitrarily assigned.

2-[4-[1-(2,6-dioxo-3-piperidyl)-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 25, 50 mg, 72.8 μmol) was submitted to SFC preparation (column: REGIS(S,S) WHELK-01 (250 mm*25 mm, 10 m); mobile phase: EtOH (0.1% NH₃H₂O); B %: 65%-65%, 4.7; 105 mins) to give fast eluting fraction (peak 1) and late eluting fraction (peak 2). HCOOH was added in the collection container before the fast eluting fraction was eluted out. The elution solvent was concentrated at 40° C. and lyophilized to give 2-[4-[1-[(3R)-2,6-dioxo-3-piperidyl]-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 26, 10 mg, 81% purity, >99% e.e.) as a white solid.

LCMS m/z (ESI): 687.3 [M+H]⁺

¹H NMR (400 MHz, CDCl₃) δ 11.12 (s, 1H) 10.41 (s, 1H) 9.78 (br s, 1H) 8.15 (s, 1H) 7.84 (d, J=9.41 Hz, 1H) 7.67 (br d, J=7.34 Hz, 2H) 7.41-7.56 (m, 4H) 7.36 (br d, J=8.07 Hz, 1H) 7.15-7.24 (m, 2H) 7.08 (s, 1H) 6.95 (d, J=7.70 Hz, 2H) 5.33-5.49 (m, 1H) 4.09 (s, 2H) 3.75 (s, 2H) 3.41 (s, 3H) 2.85-3.02 (m, 1H) 2.72-2.82 (m, 1H) 2.58-2.67 (m, 1H) 1.98-2.11 (m, 1H)

The fraction 2 was concentrated without neutralization with HCOOH at 40° C. to give 2-[4-[1-[(3S)-2,6-dioxo-3-piperidyl]-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 27, 6 mg, 97% purity, 90% e.e.) as a white solid.

LCMS m/z (ESI): 687.3 [M+H]⁺.

¹H NMR (400 MHz, CDCl₃) δ 11.13 (s, 1H) 10.25-10.54 (m, 2H) 8.18 (s, 1H) 7.87 (d, J=8.93 Hz, 1H) 7.67 (d, J=8.19 Hz, 2H) 7.40-7.57 (m, 4H) 7.35 (br d, J=8.31 Hz, 1H) 7.19 (d, J=8.19 Hz, 1H) 6.95 (s, 1H) 5.40 (dd, J=12.72, 5.14 Hz, 1H) 4.30-4.46 (m, 2H) 3.74 (br s, 2H) 3.41 (s, 3H) 2.85-2.97 (m, 1H) 2.71-2.81 (m, 1H) 2.60-2.68 (m, 1H) 1.98-2.11 (m, 1H).

### 2-[4-[1-(2,6-dioxo-3-piperidyl)-3-methyl-2-oxo-benzimidazol-5-yl]-3-methyl-phenyl]-N-[5-fluoro-7-hydroxy-6-(1,1,4-trioxo-1,2,5-thiadiazolidin-2-yl)-2-naphthyl]acetamide (Degrader 28)

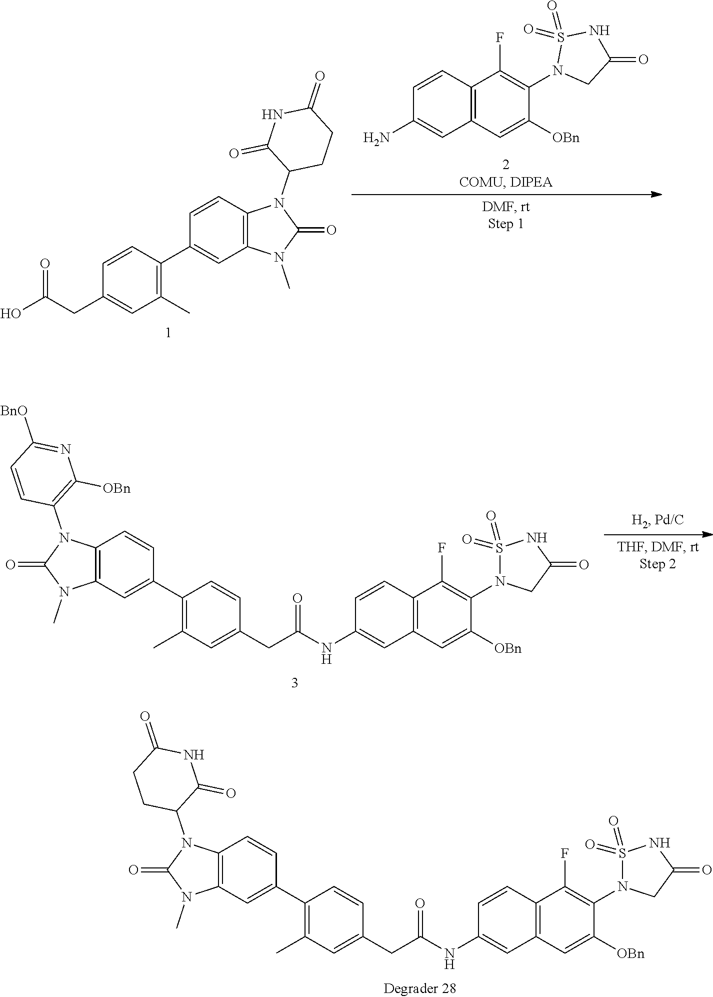 [SMILES: Cc1cc(CC(=O)O)ccc1-c1ccc2c(c1)n(C)c(=O)n2C1CCC(=O)NC1=O ; Nc1ccc2c(F)c(N3CC(=O)NS3(=O)=O)c(OCc3ccccc3)cc2c1 ; Cc1cc(CC(=O)Nc2ccc3c(F)c(N4CC(=O)NS4(=O)=O)c(OCc4ccccc4)cc3c2)ccc1-c1ccc2c(c1)n(C)c(=O)n2-c1ccc(OCc2ccccc2)nc1OCc1ccccc1 ; Cc1cc(CC(=O)Nc2ccc3c(F)c(N4CC(=O)NS4(=O)=O)c(OCc4ccccc4)cc3c2)ccc1-c1ccc2c(c1)n(C)c(=O)n2C1CCC(=O)NC1=O]

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3-methyl-phenyl]acetamide (3)

To a solution of 2-[4-[1-(2,6-dioxo-3-piperidyl)-3-methyl-2-oxo-benzimidazol-5-yl]-3-methyl-phenyl]acetic acid (1, 150 mg, 337.93 μmol, 021), 5-(6-amino-3-benzyloxy-1-fluoro-2-naphthyl)-1,1-dioxo-1,2,5-thiadiazolidin-3-one (2, 135.65 mg, 337.93 μmol) in DMF (2 mL) was added N-ethyl-N-isopropyl-propan-2-amine (218.38 mg, 1.69 mmol, 294.31 μL) and [[(Z)-(1-cyano-2-ethoxy-2-oxo-ethylidene)amino]oxy-morpholino-methylene]-dimethyl-ammonium; hexafluorophosphate (159.20 mg, 371.72 μmol). After 16 h, the mixture was adjusted with 2N HCl (5 mL) to pH=. The precipitate was filtered and purified by reversed phase flash chromatography (flow: 40 mL/min; gradient: 5%-40%, 15 min; 48%, 4 min[Water (0.1% TFA): Acetonitrile] in MeCN; column: I.D. 21 mm*H195 mm, Welch Ultimate XB_C18 20-40 μm; 120 Å) to afford N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3-methyl-phenyl]acetamide (3, 100 mg, 121.39 μmol, 36% yield) as a yellow solid.

LCMS (ES+): m/z 791.2 [M+H]⁺

### Step 2: 2-[4-[1-(2,6-dioxo-3-piperidyl)-3-methyl-2-oxo-benzimidazol-5-yl]-3-methyl-phenyl]-N-[5-fluoro-7-hydroxy-6-(1,1,4-trioxo-1,2,5-thiadiazolidin-2-yl)-2-naphthyl]acetamide (Degrader 28)

To a solution of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3-methyl-phenyl]acetamide (3, 100 mg, 101.16 μmol) in THF (1 mL) and DMF (1 mL) were added Pd(OH)₂/C (10 mg, 10% purity), Pd/C (10 mg, 10% purity), the mixture was stirred at 20° C. for 16 h under H₂ (15 psi). The mixture was filtered, concentrated and purified by reversed phase flash chromatography (flow: 40 mL/min; gradient: 5%-48% MeCN in Water (0.1% TFA) over 11 min; column: I.D. 21 mm*H195 mm, Welch Ultimate XB_C18 20-40 μm; 120 Å) to afford 2-[4-[1-(2,6-dioxo-3-piperidyl)-3-methyl-2-oxo-benzimidazol-5-yl]-3-methyl-phenyl]-N-[5-fluoro-7-hydroxy-6-(1,1,4-trioxo-1,2,5-thiadiazolidin-2-yl)-2-naphthyl]acetamide (Degrader 28, 14 mg, 16.84 μmol, 17% yield, TFA salt) as a white solid.

LCMS (ES+): m/z 701.3 [M+H]⁺

¹H NMR (400 MHz, DMSO-d₆) δ=11.11 (s, 1H), 10.45-10.22 (m, 2H), 8.18 (s, 1H), 7.86 (d, J=8.8 Hz, 1H), 7.50-7.42 (m, 1H), 7.30-7.22 (m, 2H), 7.20-7.15 (m, 3H), 7.01-6.95 (m, 2H), 5.40 (dd, J=5.2, 12.9 Hz, 1H), 4.35 (s, 2H), 3.70 (s, 2H), 3.35 (s, 3H), 2.93-2.87 (m, 1H), 2.79-2.71 (m, 1H), 2.61 (d, J=3.2 Hz, 1H), 2.26 (s, 3H), 2.10-2.01 (m, 1H)

### N-(6-(1,1-dioxido-4-oxo-1,2,5-thiadiazolidin-2-yl)-5-fluoro-7-hydroxynaphthalen-2-yl)-2-(5-(1-(2,6-dioxopiperidin-3-yl)-3-methyl-2-oxo-2,3-dihydro-1H-benzo[d]imidazol-5-yl)-1H-indazol-1-yl)acetamide (Degrader 29)

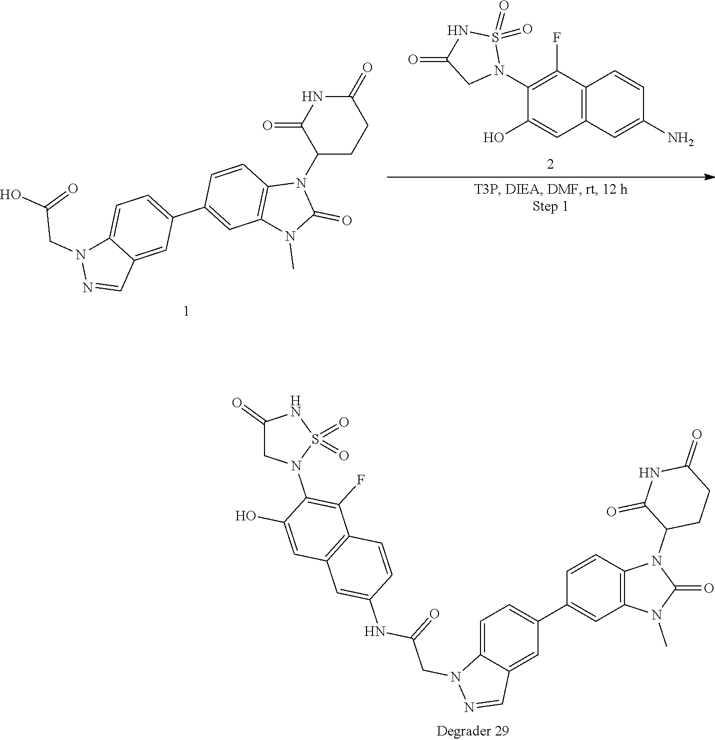

### Step 1: N-(6-(1,1-dioxido-4-oxo-1,2,5-thiadiazolidin-2-yl)-5-fluoro-7-hydroxynaphthalen-2-yl)-2-(5-(1-(2,6-dioxopiperidin-3-yl)-3-methyl-2-oxo-2,3-dihydro-1H-benzo[d]imidazol-5-yl)-1H-indazol-1-yl)acetamide (Degrader 29)

To a solution of 2-(5-(1-(2,6-dioxopiperidin-3-yl)-3-methyl-2-oxo-2,3-dihydro-1H-benzo[d]imidazol-5-yl)-1H-indazol-1-yl)acetic acid (16, 70 mg, 161.51 μmol, 1 eq) in DMF (3 mL) were added 5-(6-amino-1-fluoro-3-hydroxynaphthalen-2-yl)-1,2,5-thiadiazolidin-3-one 1,1-dioxide (2, 140 mg, 403 μmol, HCl salt, 2.49 eq), T₃P (130 μmL, 807 μmol, 50 purity in EtOAc, 5 eq) and DIEA (208 mg, 1.62 mmol, 281 μL, 10 eq). The mixture was stirred at 25° C. for 12 h. The reaction was filtered, the filtrate was purified by prep-HPLC Prep-HPLC (flow: 28 mL/min; gradient: from 29-49% water (0.05% HCl) in MeCN over 6.5 min; column: 3_PhenomenexLuna C18 75×30 mm×3 μm) followed by prep-HPLC (flow: 30 mL/min; gradient: from 6-30% water in MeCN over 8 min; column: 3_PhenomenexLuna C18 75×30 mm×3 μm) to give N-(6-(1,1-dioxido-4-oxo-1,2,5-thiadiazolidin-2-yl)-5-fluoro-7-hydroxynaphthalen-2-yl)-2-(5-(1-(2,6-dioxopiperidin-3-yl)-3-methyl-2-oxo-2,3-dihydro-1H-benzo[d]imidazol-5-yl)-1H-indazol-1-yl)acetamide (Degrader 29, 5.88 mg, 7.77 μmol, 5% yield) as a red solid.

LC-MS (ES+): m/z 727.3 [M+H]⁺

¹H NMR (400 MHz, DMSO-d₆) δ=11.21-11.02 (br s, 1H), 10.67 (s, 1H), 9.77-9.49 (m, 1H), 8.17 (s, 1H), 8.12 (s, 1H), 8.06 (s, 1H), 7.86 (d, J=8.8 Hz, 1H), 7.82-7.75 (m, 2H), 7.57 (d, J=1.6 Hz, 1H), 7.48-7.38 (m, 2H), 7.22 (d, J=8.4 Hz, 1H), 6.92 (s, 1H), 5.47-5.37 (m, 3H), 4.06 (s, 2H), 3.43 (s, 3H), 2.99-2.86 (m, 1H), 2.83-2.69 (m, 1H), 2.69-2.60 (m, 1H), 2.07 (dd, J=5.2, 10.0 Hz, 1H).

### N-(6-(1,1-dioxido-4-oxo-1,2,5-thiadiazolidin-2-yl)-5-fluoro-7-hydroxynaphthalen-2-yl)-3-(4-(3-(2,6-dioxopiperidin-3-yl)-1-methyl-1H-indazol-6-yl)piperidin-1-yl)cyclobutanecarboxamide (Degrader 30)

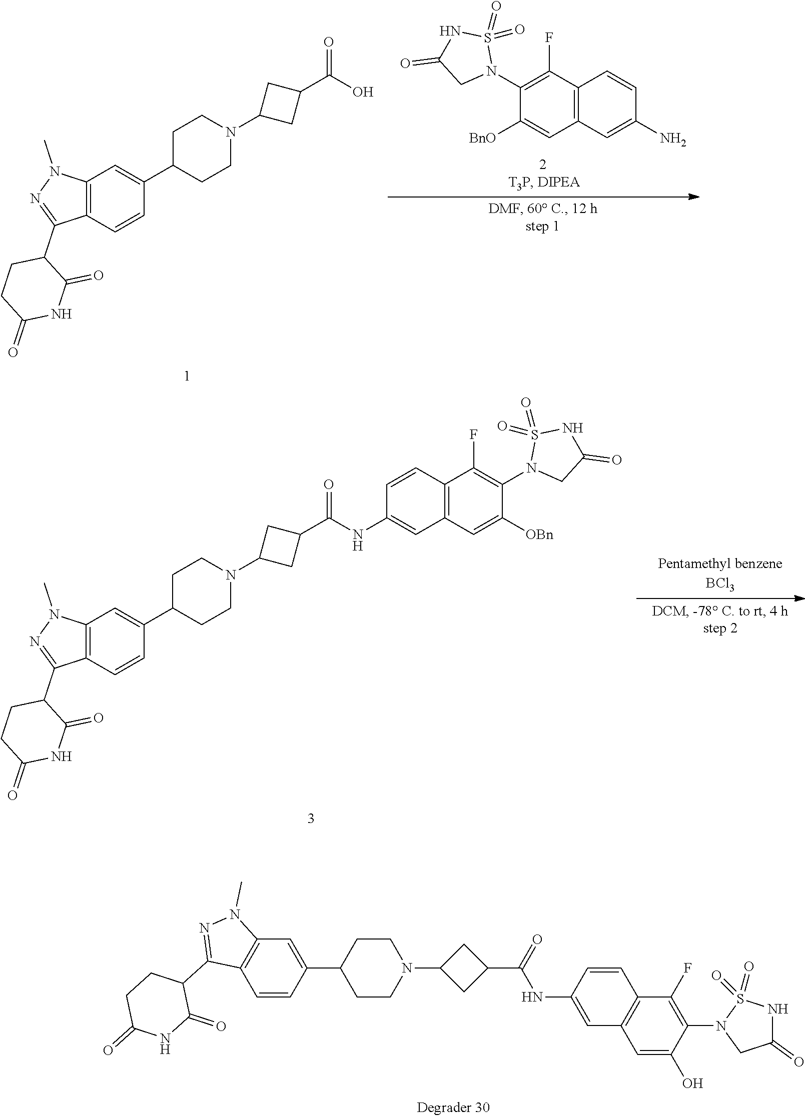 [SMILES: Cn1nc(C2CCC(=O)NC2=O)c2ccc(C3CCN(C4CC(C(=O)O)C4)CC3)cc21 ; Nc1ccc2c(F)c(N3CC(=O)NS3(=O)=O)c(OCc3ccccc3)cc2c1 ; Cn1nc(C2CCC(=O)NC2=O)c2ccc(C3CCN(C4CC(C(=O)Nc5ccc6c(F)c(N7CC(=O)NS7(=O)=O)c(OCc7ccccc7)cc6c5)C4)CC3)cc21 ; Cn1nc(C2CCC(=O)NC2=O)c2ccc(C3CCN(C4CC(C(=O)Nc5ccc6c(F)c(N7CC(=O)NS7(=O)=O)c(O)cc6c5)C4)CC3)cc21]

### Step 1: N-(7-(benzyloxy)-6-(1,1-dioxido-4-oxo-1,2,5-thiadiazolidin-2-yl)-5-fluoronaphthalen-2-yl)-3-(4-(3-(2,6-dioxopiperidin-3-yl)-1-methyl-1H-indazol-6-yl)piperidin-1-yl)cyclobutanecarboxamide (3)

To a solution of 3-(4-(3-(2,6-dioxopiperidin-3-yl)-1-methyl-1H-indazol-6-yl)piperidin-1-yl)cyclobutanecarboxylic acid (1, 200 mg, 433.88 μmol, HCl salt, 1 eq) and 5-(6-amino-3-(benzyloxy)-1-fluoronaphthalen-2-yl)-1,2,5-thiadiazolidin-3-one 1,1-dioxide (2, 174.17 mg, 433.88 μmol, TFA salt, 1 eq) in DMF (4 mL) were added DIPEA (841.15 mg, 6.51 mmol, 1.13 mL, 15 eq) and T₃P (966.38 mg, 3.04 mmol, 50% purity in ethyl acetate, 7 eq). The mixture was stirred at 50° C. for 16 h. The reaction mixture was filtered and concentrated under reduced pressure. The residue was purified by prep-HPLC (flow: 25 mL/min; gradient: from 26-56% MeCN-water (0.1% TFA) over 7 min; column: 3_Phenomenex Luna C18 75×30 mm×3 um) to afford N-(7-(benzyloxy)-6-(1,1-dioxido-4-oxo-1,2,5-thiadiazolidin-2-yl)-5-fluoronaphthalen-2-yl)-3-(4-(3-(2,6-dioxopiperidin-3-yl)-1-methyl-1H-indazol-6-yl)piperidin-1-yl)cyclobutanecarboxamide (3, 120 mg, 128.86 μmol, 30% yield, TFA salt) as a white solid.

LCMS (ESI): m/z 808.5 [M+H]⁺

### Step 2: N-(6-(1,1-dioxido-4-oxo-1,2,5-thiadiazolidin-2-yl)-5-fluoro-7-hydroxynaphthalen-2-yl)-3-(4-(3-(2,6-dioxopiperidin-3-yl)-1-methyl-1H-indazol-6-yl)piperidin-1-yl)cyclobutanecarboxamide (Degrader 30)

To a solution of N-(7-(benzyloxy)-6-(1,1-dioxido-4-oxo-1,2,5-thiadiazolidin-2-yl)-5-fluoronaphthalen-2-yl)-3-(4-(3-(2,6-dioxopiperidin-3-yl)-1-methyl-1H-indazol-6-yl)piperidin-1-yl)cyclobutanecarboxamide (3, 120 mg, 148.54 μmol, TFA salt, 1 eq) in DCM (5 mL) was added 1,2,3,5-pentamethylbenzene (66.06 mg, 445.61 μmol, 3 eq). Then added BCl₃ (1 M in DCM, 4.46 mL, 30 eq) at −78° C. The mixture was stirred at 20° C. for 4 h. The reaction mixture was quenched by addition of DCM:MeOH=10:1 (10 mL) at −78° C. Then the mixture was filtered and concentrated under reduced pressure. The residue was purified by prep-HPLC (flow: 25 mL/min; gradient: from 15-45% MeCN-water (0.1% TFA) over 7 min; column: 3_Phenomenex Luna C18 75×30 mm×3 um) to afford N-(6-(1,1-dioxido-4-oxo-1,2,5-thiadiazolidin-2-yl)-5-fluoro-7-hydroxynaphthalen-2-yl)-3-(4-(3-(2,6-dioxopiperidin-3-yl)-1-methyl-1H-indazol-6-yl)piperidin-1-yl)cyclobutanecarboxamide (Degrader 30, 63.17 mg, 75.94 μmol, 51% yield, TFA salt) as an off-white solid.

LCMS (ESI): m/z 718.2 [M+H]⁺

¹H NMR (400 MHz, DMSO-d₆) δ 10.89 (s, 1H), 10.24 (s, 1H), 10.00-9.74 (m, 1H), 9.66-9.24 (m, 1H), 8.25-8.09 (m, 1H), 7.85 (d, J=9.2 Hz, 1H), 7.69 (d, J=8.4 Hz, 1H), 7.54-7.37 (m, 2H), 7.09-6.91 (m, 2H), 4.35 (dd, J=5.2, 9.2 Hz, 1H), 4.15 (s, 2H), 3.99 (s, 4H), 3.73 (d, J=6.8 Hz, 1H), 3.54 (d, J=10.0 Hz, 2H), 3.29-3.21 (m, 1H), 3.10-2.92 (m, 4H), 2.74-2.58 (m, 4H), 2.37-2.30 (m, 1H), 2.20-2.07 (m, 3H), 2.00-1.88 (m, 2H).

### 2-[4-[4-chloro-1-(2,6-dioxo-3-piperidyl)-3-methyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]acetamide (Degrader 31)

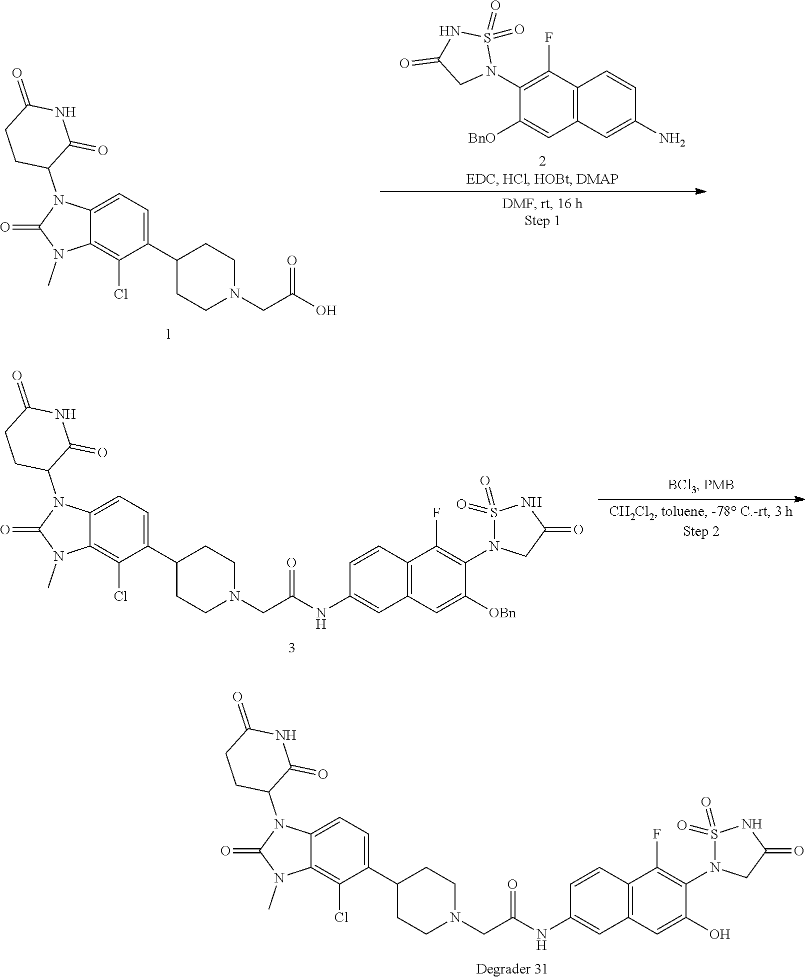 [SMILES: Cn1c(=O)n(C2CCC(=O)NC2=O)c2ccc(C3CCN(CC(=O)O)CC3)c(Cl)c21 ; Nc1ccc2c(F)c(N3CC(=O)NS3(=O)=O)c(OCc3ccccc3)cc2c1 ; Cn1c(=O)n(C2CCC(=O)NC2=O)c2ccc(C3CCN(CC(=O)Nc4ccc5c(F)c(N6CC(=O)NS6(=O)=O)c(OCc6ccccc6)cc5c4)CC3)c(Cl)c21 ; Cn1c(=O)n(C2CCC(=O)NC2=O)c2ccc(C3CCN(CC(=O)Nc4ccc5c(F)c(N6CC(=O)NS6(=O)=O)c(O)cc5c4)CC3)c(Cl)c21]

### Step 1: N-[7-benzyloxy-5-fluoro-6-(1,1,4-trioxo-1,2,5-thiadiazolidin-2-yl)-2-naphthyl]-2-[4-[4-chloro-1-(2,6-dioxo-3-piperidyl)-3-methyl-2-oxo-benzimidazol-5-yl]-1-piperidyl]acetamide (3)

Into a 25 mL round-bottom flask containing a well-stirred solution of 2-[4-[4-chloro-1-(2,6-dioxo-3-piperidyl)-3-methyl-2-oxo-benzimidazol-5-yl]-1-piperidyl]acetic acid (1, 110.16 mg, 174.61 μmol, TFA salt) and 5-(6-amino-3-benzyloxy-1-fluoro-2-naphthyl)-1,1-dioxo-1,2,5-thiadiazolidin-3-one (2, 100 mg, 174.61 μmol, TFA salt) in DMF (1.00 mL) was added EDC·HCl (100.42 mg, 523.83 μmol), HOBt (47.19 mg, 349.22 μmol) and DMAP (127.99 mg, 1.05 mmol). The resulting reaction mixture was stirred at room temperature for 16 h. Afterwards, solvent was removed under reduced pressure and ice-cold water was added to precipitate a solid that was filtered, washed with water and dried under vacuum to get N-[7-benzyloxy-5-fluoro-6-(1,1,4-trioxo-1,2,5-thiadiazolidin-2-yl)-2-naphthyl]-2-[4-[4-chloro-1-(2,6-dioxo-3-piperidyl)-3-methyl-2-oxo-benzimidazol-5-yl]-1-piperidyl]acetamide (3, 120 mg, 74.79 μmol, 43% yield) as an off-white solid.

LCMS (ES+): m/z 818.0 [M+H]⁺

### Step 2: 2-[4-[4-chloro-1-(2,6-dioxo-3-piperidyl)-3-methyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]acetamide (Degrader 31)

Into a 50 mL round-bottom flask containing a well-stirred solution of N-[7-benzyloxy-5-fluoro-6-(1,1,4-trioxo-1,2,5-thiadiazolidin-2-yl)-2-naphthyl]-2-[4-[4-chloro-1-(2,6-dioxo-3-piperidyl)-3-methyl-2-oxo-benzimidazol-5-yl]-1-piperidyl]acetamide (3, 120 mg, 74.79 μmol) in DCM (2 mL) and toluene (2 mL) was added pentamethylbenzene (55.44 mg, 373.96 μmol, 60.46 μL) and the reaction mixture was cooled to −78° C. Then BCl₃ (1.0 M solution in DCM, 1.50 mmol, 1.50 mL) was added dropwise over a period of 2 min. Subsequently the reaction mixture was brought to room temperature and stirred for 3 h. The reaction mixture was cooled to −78° C. and quenched slowly with 5% MeOH in DCM (1.0 mL). The reaction mixture was allowed to attain room temperature and concentrated under reduced pressure at 30° C. to get the crude compound that was purified by reverse-phase preparative HPLC [Column: X-Select, C18 (150×19) mm, 5 μm; Mobile phase A: 0.1% TFA in water and Mobile phase B: CH₃CN] to get 2-[4-[4-chloro-1-(2,6-dioxo-3-piperidyl)-3-methyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]acetamide (Degrader 31, 35 mg, 39.46 μmol, 53% yield, TFA salt) as an off-white solid.

1H-NMR (400 MHz, DMSO-d6): δ 11.16 (s, 1H), 10.81 (s, 1H), 9.92 (s, 1H), 9.86 (brs, 1H), 8.12 (s, 1H), 7.90 (d, J=8.80 Hz, 1H), 7.46 (dd, J=1.60, 9.00 Hz, 1H), 7.17 (d, J=8.00 Hz, 1H), 7.03-6.98 (m, 2H), 5.41 (dd, J=5.20, 12.80 Hz, 1H), 4.23 (s, 2H), 4.11 (s, 2H), 3.75-3.60 (m, 5H), 2.94-2.86 (m, 1H), 2.74-2.66 (m, 3H), 2.62-2.55 (m, 2H), 2.10-1.95 (m, 5H).

LCMS (ES−): m/z 726.0 [M−H]⁻

### 2-[4-[1-(2,6-dioxo-3-piperidyl)-3-methyl-2-oxo-benzimidazol-5-yl]-2-oxo-1-pyridyl]-N-[5-fluoro-7-hydroxy-6-(1,1,4-trioxo-1,2,5-thiadiazolidin-2-yl)-2-naphthyl]acetamide (Degrader 32)

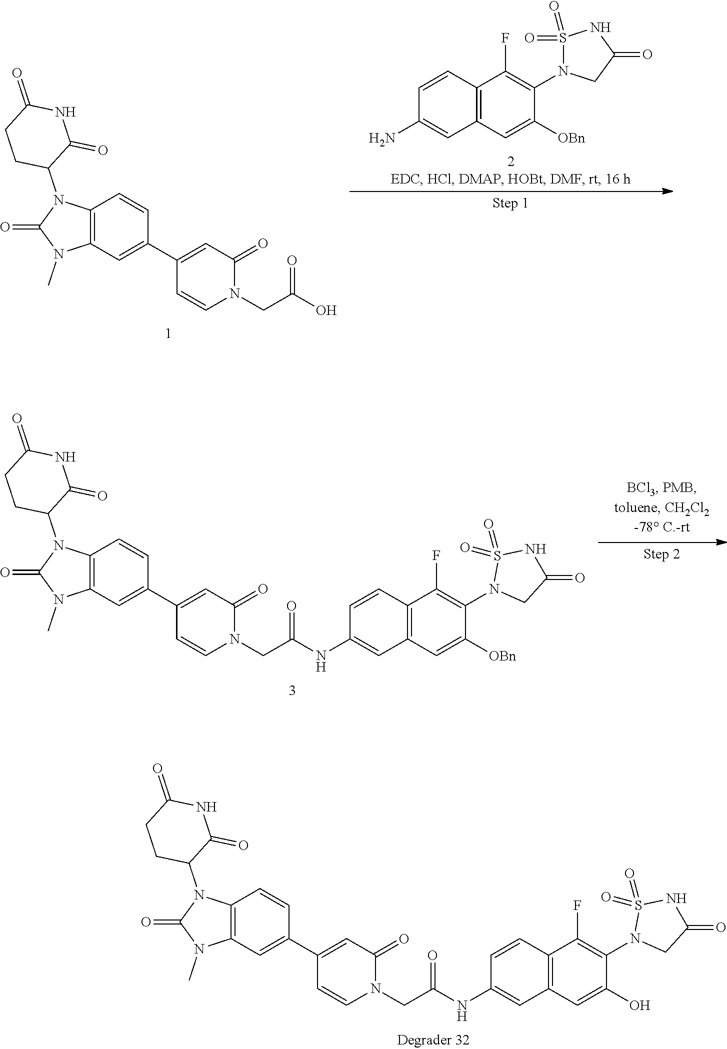 [SMILES: Cn1c(=O)n(C2CCC(=O)NC2=O)c2ccc(-c3ccn(CC(=O)O)c(=O)c3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(-c3ccn(CC(=O)Nc4ccc5c(F)c(N6CC(=O)NS6(=O)=O)c(OCc6ccccc6)cc5c4)c(=O)c3)cc21 ; Cn1c(=O)n(C2CCC(=O)NC2=O)c2ccc(-c3ccn(CC(=O)Nc4ccc5c(F)c(N6CC(=O)NS6(=O)=O)c(O)cc5c4)c(=O)c3)cc21]

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-2-oxo-1-pyridyl]acetamide (3)

Into a 25 mL single-neck round-bottom flask containing a well-stirred solution of 2-[4-[1-(2,6-dioxo-3-piperidyl)-3-methyl-2-oxo-benzimidazol-5-yl]-2-oxo-1-pyridyl]acetic acid (1, 0.41 g, 849.21 μmol) and 5-(6-amino-3-benzyloxy-1-fluoro-2-naphthyl)-1,1-dioxo-1,2,5-thiadiazolidin-3-one (2, 437.71 mg, 849.21 μmol, TFA salt) in anhydrous DMF (7 mL) were added EDC·HCl (395.50 mg, 2.55 mmol), hydroxybenzotriazole (229.49 mg, 1.70 mmol) and 4-dimethylaminopyridine (622.49 mg, 5.10 mmol) at room temperature. The resulting mixture was stirred at room temperature. After 16 h, the reaction mixture was purified by reverse phase column chromatography [Purification method: Biotage C18, 100 g column; Mobile phase A: 0.1% TFA in water; Mobile phase B: MeCN] to obtain N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-2-oxo-1-pyridyl]acetamide (3, 0.49 g, 302.17 μmol, 36% yield) as a light brown solid.

LCMS (ES+): m/z 794.0 [M+H]⁺

### Step 2: 2-[4-[1-(2,6-dioxo-3-piperidyl)-3-methyl-2-oxo-benzimidazol-5-yl]-2-oxo-1-pyridyl]-N-[5-fluoro-7-hydroxy-6-(1,1,4-trioxo-1,2,5-thiadiazolidin-2-yl)-2-naphthyl]acetamide (Degrader 32)

Into a 50 mL single-neck round bottom flask containing a well-stirred solution of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-2-oxo-1-pyridyl]acetamide (3, 0.49 g, 302.48 μmol) and pentamethylbenzene (224.20 mg, 1.51 mmol, 244.50 μL) in anhydrous toluene (3 mL) and DCM (3 mL) was added a 1.0 M solution of boron trichloride in DCM (9.5 mmol, 9.5 mL) at −78° C. The resultant reaction mixture was stirred at ambient temperature for 16 h. The reaction mixture was quenched with 5% MeOH in DCM (3 mL) at −78° C. The solvents were removed from the reaction mixture under reduced pressure. The residue was triturated with MTBE (2×10 mL), filtered and dried. The crude material was purified by reverse phase prep HPLC [Purification method: Column: X-Select, C18 (150×10) mm, 5 micron; Mobile phase A: 0.1% TFA in water and Mobile phase B: MeCN] to afford 2-[4-[1-(2,6-dioxo-3-piperidyl)-3-methyl-2-oxo-benzimidazol-5-yl]-2-oxo-1-pyridyl]-N-[5-fluoro-7-hydroxy-6-(1,1,4-trioxo-1,2,5-thiadiazolidin-2-yl)-2-naphthyl]acetamide (Degrader 32, 54 mg, 70.36 μmol, 23% yield) as an off-white solid.

LCMS (ES−): m/z 702.0 [M−H]⁻

¹HNMR (400 MHz, DMSO-d6): δ 11.16 (s, 1H), 10.64 (s, 1H), 10.35 (s, 1H), 8.19 (s, 1H), 7.90 (d, J=9.20 Hz, 1H), 7.78 (d, J=7.20 Hz, 1H), 7.67 (s, 1H), 7.50 (d, J=8.40 Hz, 1H), 7.45 (d, J=8.80 Hz, 1H), 7.25 (d, J=8.40 Hz, 1H), 6.97 (s, 1H), 6.81 (d, J=1.60 Hz, 1H), 6.74 (d, J=7.20 Hz, 1H), 5.46-5.42 (m, 1H), 4.84 (s, 2H), 4.36 (s, 2H), 3.44 (s, 3H), 2.92-2.85 (m, 1H), 2.78-2.74 (m, 1H), 2.68-2.63 (m, 2H), 2.08-2.05 (m, 1H).

N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-2-fluorophenyl)acetamide (Degrader 33)

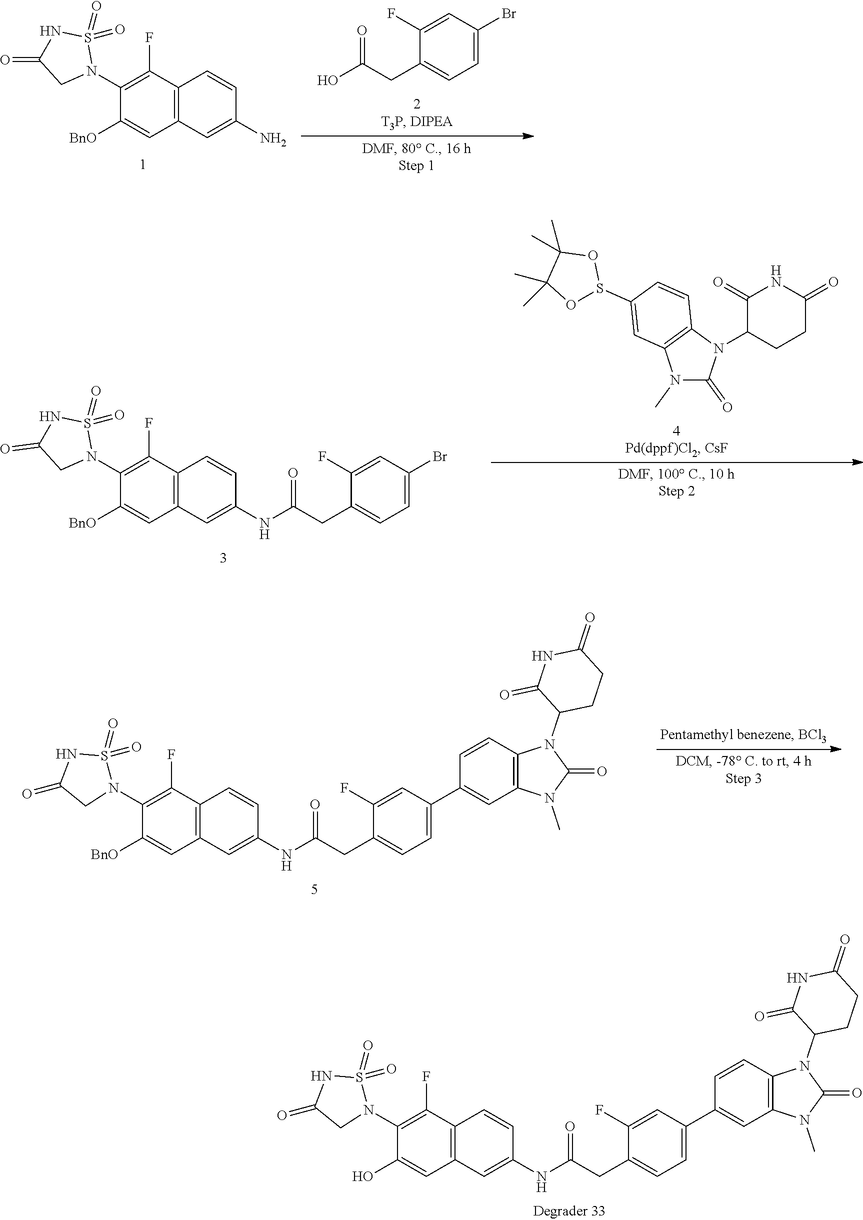

### Step 1: N-(7-(benzyloxy)-6-(1,1-dioxido-4-oxo-1,2,5-thiadiazolidin-2-yl)-5-fluoronaphthalen-2-yl)-2-(4-bromo-2-fluorophenyl)acetamide (3)

To a solution of 5-(6-amino-3-(benzyloxy)-1-fluoronaphthalen-2-yl)-1,2,5-thiadiazolidin-3-one 1,1-dioxide (1, 500 mg, 970.06 μmol, TFA salt, 1 eq) in DMF (5 mL) was added 2-(4-bromo-2-fluorophenyl)acetic acid (2, 226.06 mg, 970.06 μmol, 1 eq), DIPEA (1.88 g, 14.55 mmol, 2.53 mL, 15 eq and T₃P (4.32 g, 6.79 mmol, 50% purity in EtOAc, 7 eq. The mixture was stirred at 50° C. for 16 h. The mixture was filtered and concentrated in vacuum. The residue was purified by flash silica gel chromatography (SiO₂, DCM:MeOH=1:0 to 10:1) followed by reversed phase flash (flow: 35 mL/min; gradient: from 0-75% MeCN in water (0.1% TFA) over 30 min; column: Welch Ultimate XB-C18, 20-40 M, 120 Å, I.D. 95 mm×H 365 mm) to afford N-(7-(benzyloxy)-6-(1,1-dioxido-4-oxo-1,2,5-thiadiazolidin-2-yl)-5-fluoronaphthalen-2-yl)-2-(4-bromo-2-fluorophenyl)acetamide (3, 220 mg, 353.32 μmol, 36% yield) as a white solid.

LCMS (ESI): m/z 618.0 [M+H]⁺

### Step 2: N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)-2-fluorophenyl)acetamide (5)

A mixture of N-(7-(benzyloxy)-6-(1,1-dioxido-4-oxo-1,2,5-thiadiazolidin-2-yl)-5-fluoronaphthalen-2-yl)-2-(4-bromo-2-fluorophenyl)acetamide (3, 90 mg, 146.00 μmol, 1 eq), 3-(3-methyl-2-oxo-5-(4,4,5,5-tetramethyl-1,3,2-dioxaborolan-2-yl)-2,3-dihydro-1H-benzo[d]imidazol-1-yl)piperidine-2,6-dione (4, 61.87 mg, 160.60 μmol, 1.1 eq), Pd(dppf)Cl₂ (5.34 mg, 7.30 μmol, 0.05 eq) and CsF (66.53 mg, 438.01 μmol, 3 eq) in DMF (2 mL) was degassed and purged with N₂ 3 times, and then the mixture was stirred at 100° C. for 16 h under N₂ atmosphere. The mixture was filtered and concentrated in vacuum. The residue was purified by prep-HPLC (flow: 25 mL/min; gradient: from 45-75% MeCN in water (0.1% TFA) over 9 min; column: 3_Phenomenex Luna C18 75×30 mm×3 um) to afford N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)-2-fluorophenyl)acetamide (5, 40 mg, 44.29 μmol, 30% yield) as a white solid.

LCMS (ESI): m/z 795.2 [M+H]⁺

### Step 3: N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-2-fluorophenyl)acetamide (Degrader 33)

To a solution of N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)-2-fluorophenyl)acetamide (5, 70 mg, 88.07 μmol, 1 eq) and 1,2,3,4,5-pentamethylbenzene (39.17 mg, 264.22 μmol, 42.72 μL, 3 eq) in DCM (2 mL) were added BCl₃ (1 M in DCM, 2.64 mL, 30 eq) at −78° C. The mixture was stirred at 20° C. for 4 h. A solution of DCM:MeOH=10:1 was added at −78° C., and the mixture was concentrated in vacuum. The residue was purified by prep-HPLC (flow: 30 mL/min; gradient: from 8-38% MeCN in water over 10 min; column: Phenomenex Gemini-NX C18 75×30 mm×3 um) to afford N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-2-fluorophenyl)acetamide (Degrader 33, 18.06 mg, 24.86 μmol, 28% yield) as a pink solid.

LCMS (ESI): m/z 705.3 [M+H]⁺

¹H NMR (400 MHz, DMSO) δ 11.12 (s, 1H), 10.44 (s, 1H), 9.55 (s, 1H), 8.14 (s, 1H), 7.84 (d, J=9.0 Hz, 1H), 7.61-7.57 (m, 1H), 7.57-7.52 (m, 1H), 7.50 (d, J=7.9 Hz, 1H), 7.47-7.39 (m, 2H), 7.21 (d, J=8.3 Hz, 1H), 6.93 (s, 1H), 5.41 (dd, J=12.8, 5.4 Hz, 1H), 4.06 (s, 2H), 3.83 (s, 2H), 3.42 (s, 3H), 2.98-2.86 (m, 1H), 2.83-2.59 (m, 2H), 2.11-1.99 (m, 1H).

### 4-[[1-(2,6-dioxo-3-piperidyl)-3-methyl-2-oxo-benzimidazol-5-yl]methyl]-N-[5-fluoro-7-hydroxy-6-(1,1,4-trioxo-1,2,5-thiadiazolidin-2-yl)-2-naphthyl]piperidine-1-carboxamide (Degrader 34)

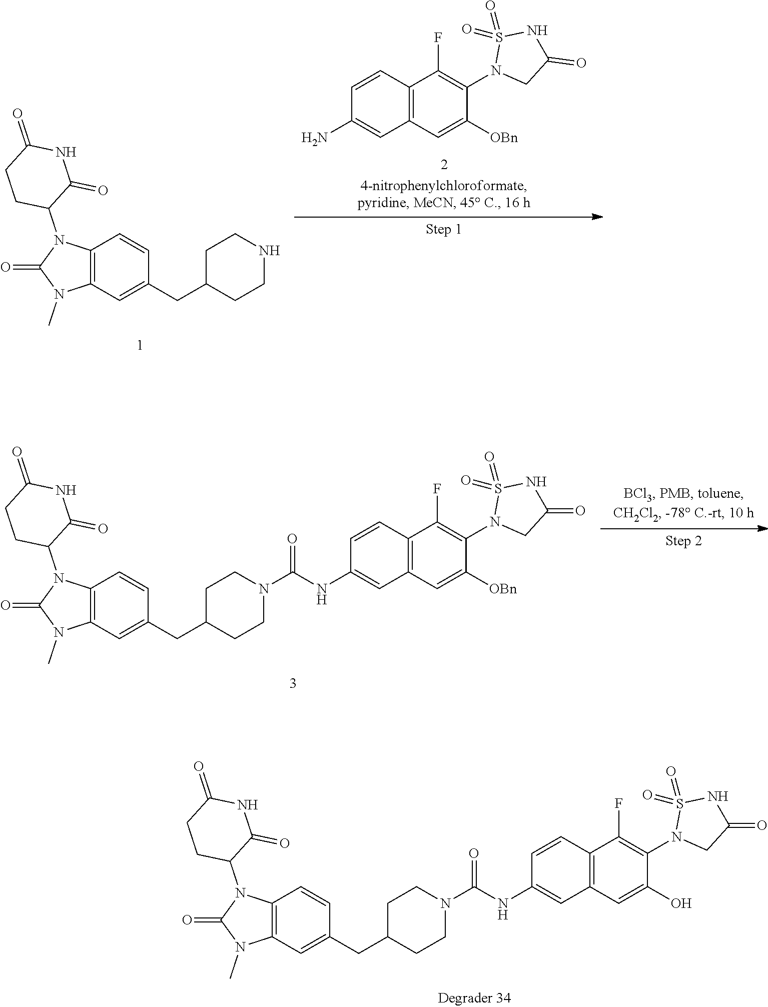 [SMILES: Cn1c(=O)n(C2CCC(=O)NC2=O)c2ccc(CC3CCNCC3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(CC3CCN(C(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(CC3CCN(C(=O)Nc4ccc5c(F)c(N6CC(=O)NS6(=O)=O)c(O)cc5c4)CC3)cc21]

### Step 1: N-[7-benzyloxy-5-fluoro-6-(1,1,4-trioxo-1,2,5-thiadiazolidin-2-yl)-2-naphthyl]-4-[[1-(2,6-dioxo-3-piperidyl)-3-methyl-2-oxo-benzimidazol-5-yl]methyl]piperidine-1-carboxamide (3)

Into a 100 mL single neck round bottom flask containing a well-stirred solution of 5-(6-amino-3-benzyloxy-1-fluoro-2-naphthyl)-1,1-dioxo-1,2,5-thiadiazolidin-3-one (2, 550 mg, 1.28 mmol) in anhydrous acetonitrile (10 mL) was added pyridine (1.01 g, 12.82 mmol, 1.04 mL) and 4-nitrophenyl chloroformate (516.77 mg, 2.56 mmol) at room temperature. The resulting solution was stirred for 2 h at room temperature. Then a solution of 3-[3-methyl-2-oxo-5-(4-piperidylmethyl)benzimidazol-1-yl]piperidine-2,6-dione (1, 532.89 mg, 1.28 mmol) in acetonitrile (1 mL) was added. The resulting mixture was heated at 45° C. After 16 h, the reaction mixture was concentrated and the residue subjected to reverse phase column chromatography (Purification method: Siliasep premium C18, 25 um, 120 g column; Mobile phase A: 0.1% TFA in water; Mobile phase B: Acetonitrile) to afford N-[7-benzyloxy-5-fluoro-6-(1,1,4-trioxo-1,2,5-thiadiazolidin-2-yl)-2-naphthyl]-4-[[1-(2,6-dioxo-3-piperidyl)-3-methyl-2-oxo-benzimidazol-5-yl]methyl]piperidine-1-carboxamide (3, 260 mg, 286.99 μmol, 22% yield) as a pale brown solid.

LCMS: m/z 784.0 [M+H]⁺

### Step 2: 4-[[1-(2,6-dioxo-3-piperidyl)-3-methyl-2-oxo-benzimidazol-5-yl]methyl]-N-[5-fluoro-7-hydroxy-6-(1,1,4-trioxo-1,2,5-thiadiazolidin-2-yl)-2-naphthyl]piperidine-1-carboxamide (Degrader 34)

Into a 100 mL round bottom flask containing a well-stirred solution of N-[7-benzyloxy-5-fluoro-6-(1,1,4-trioxo-1,2,5-thiadiazolidin-2-yl)-2-naphthyl]-4-[[1-(2,6-dioxo-3-piperidyl)-3-methyl-2-oxo-benzimidazol-5-yl]methyl]piperidine-1-carboxamide (3, 260 mg, 286.99 μmol) in toluene (4 mL) and DCM (4 mL) was added pentamethylbenzene (212.73 mg, 1.43 mmol) at room temperature. Then a 1.0 M solution of boron trichloride in DCM (1.01 g, 8.61 mmol, 8.61 mL) was added at −78° C. The reaction mixture was stirred at room temperature. After 16 h, the reaction was quenched with 5% methanol in DCM (8 mL) at −78° C. The mixture was concentrated under reduced pressure. The crude compound was purified by reverse phase prep HPLC (Purification method: column X-bridge C8 (19.1×150) mm; Mobile phase A: 0.1% TFA in water; Mobile phase B: Acetonitrile) to afford 4-[[1-(2,6-dioxo-3-piperidyl)-3-methyl-2-oxo-benzimidazol-5-yl]methyl]-N-[5-fluoro-7-hydroxy-6-(1,1,4-trioxo-1,2,5-thiadiazolidin-2-yl)-2-naphthyl]piperidine-1-carboxamide (Degrader 34, 33 mg, 39.77 μmol, 14% yield, TFA salt) as an off-white solid.

LCMS: m/z 694.0 [M+H]⁺

¹HNMR (400 MHz, DMSO-d6): δ 11.12 (s, 1H), 10.25 (bs, 1H), 8.73 (s, 1H), 7.89 (s, 1H), 7.77 (d, J=8.80 Hz, 1H), 7.49 (d, J=9.20 Hz, 1H), 7.08-7.04 (m, 2H), 6.90-6.86 (m, 2H), 5.37-5.33 (m, 1H), 4.37 (s, 2H), 4.15 (d, J=12.80 Hz, 2H), 3.37 (s, 3H), 2.94-2.88 (m, 1H), 2.86-2.73 (m, 3H), 2.70-2.57 (m, 3H), 2.03-2.00 (m, 1H), 1.76-1.62 (m, 3H), 1.19-1.16 (m, 2H).

### N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-isopropyl-2-oxo-2,3-dihydro-1H-benzo[d]imidazol-5-yl)piperidin-4-yl)acetamide (Degrader 35)

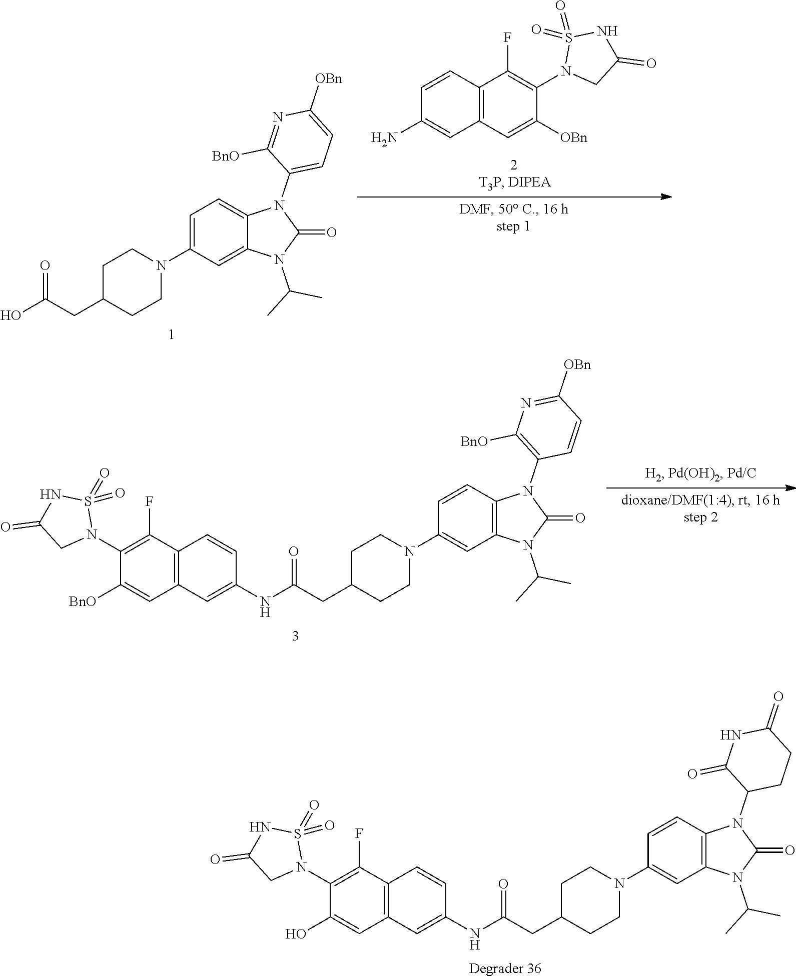 [SMILES: CC(C)n1c(=O)n(-c2ccc(OCc3ccccc3)nc2OCc2ccccc2)c2ccc(N3CCC(CC(=O)O)CC3)cc21 ; Nc1ccc2c(F)c(N3CC(=O)NS3(=O)=O)c(OCc3ccccc3)cc2c1 ; CC(C)n1c(=O)n(-c2ccc(OCc3ccccc3)nc2OCc2ccccc2)c2ccc(N3CCC(CC(=O)Nc4ccc5c(F)c(N6CC(=O)NS6(=O)=O)c(OCc6ccccc6)cc5c4)CC3)cc21 ; CC(C)n1c(=O)n(C2CCC(=O)NC2=O)c2ccc(N3CCC(CC(=O)Nc4ccc5c(F)c(N6CC(=O)NS6(=O)=O)c(O)cc5c4)CC3)cc21]

### Step 1: N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-isopropyl-2-oxo-2,3-dihydro-1H-benzo[d]imidazol-5-yl)piperidin-4-yl)acetamide (3)

To a solution of 2-(1-(1-(2,6-bis(benzyloxy)pyridin-3-yl)-3-isopropyl-2-oxo-2,3-dihydro-1H-benzo[d]imidazol-5-yl)piperidin-4-yl)acetic acid (1, 300 mg, 466.44 μmol, 1 eq) and 5-(6-amino-3-(benzyloxy)-1-fluoronaphthalen-2-yl)-1,2,5-thiadiazolidin-3-one 1,1-dioxide (2, 187.23 mg, 466.44 μmol, 1 eq) in DMF (2 mL) were added DIPEA (904.26 mg, 7.00 mmol, 1.22 mL, 15 eq and T₃P (2.08 g, 3.27 mmol, 50% purity in ethyl acetate, 7 eq. The mixture was stirred at 50° C. for 16 h. The reaction mixture was filtered and concentrated under reduced pressure. The residue was purified by prep-HPLC (flow: 25 mL/min; gradient: from 30-60% MeCN-water (0.05% ammonia hydroxide v/v) over 7 min; column: Waters X bridge 150×25 mm×5 um) to afford N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-isopropyl-2-oxo-2,3-dihydro-11H-benzo[d]imidazol-5-yl)piperidin-4-yl)acetamide (3, 150 mg, 151.50 μmol, 32% yield) as a white solid.

LCMS (ESI): m/z 990.4 [M+H]⁺

### Step 2: N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-isopropyl-2-oxo-2,3-dihydro-1H-benzo[d]imidazol-5-yl)piperidin-4-yl)acetamide (Degrader 35)

To a solution of N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-isopropyl-2-oxo-2,3-dihydro-1H-benzo[d]imidazol-5-yl)piperidin-4-yl)acetamide (3, 50 mg, 50.50 μmol, 1 eq) in dioxane (0.5 mL) and DMF (2 mL) was added Pd/C (50.00 mg, 10% purity) and Pd(OH)₂/C (50 mg, 10% purity) under N₂ atmosphere. The suspension was degassed and purged with H₂ 3 times. The mixture was stirred under H₂ (15 Psi) at 20° C. for 16 h. The reaction mixture and other batch (80 mg material) were filtered and concentrated under reduced pressure to give a residue. The residue was purified by prep-HPLC (flow: 25 mL/min; gradient: from 11-41% MeCN-water (0.1% TFA) over 10 min; column: Phenomenex Synergi C18 150×25 mm×10 um) to afford N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-isopropyl-2-oxo-2,3-dihydro-1H-benzo[d]imidazol-5-yl)piperidin-4-yl)acetamide (Degrader 35, 47.24 mg, 65.5 μmol, 49% yield, TFA salt) as an off-white solid.

LCMS (ESI): m/z 722.2 [M+H]⁺

¹H NMR (400 MHz, DMSO-d₆) δ 11.11 (s, 1H), 10.22 (s, 1H), 10.10-9.91 (m, 1H), 8.17 (s, 1H), 7.85 (d, J=8.8 Hz, 1H), 7.63-7.38 (m, 2H), 7.22-7.09 (m, 2H), 6.96 (d, J=2.4 Hz, 1H), 5.37 (dd, J=5.2, 12.8 Hz, 1H), 4.66-4.58 (m, 1H), 4.21 (s, 2H), 3.61 (d, J=10.0 Hz, 3H), 2.91-2.84 (m, 1H), 2.72-2.67 (m, 1H), 2.60 (s, 1H), 2.44 (d, J=6.4 Hz, 2H), 2.25-2.17 (m, 1H), 2.05-1.96 (m, 3H), 1.73-1.60 (m, 2H), 1.48 (d, J=7.2 Hz, 7H).

### 2-[4-[1-(2,6-dioxo-3-piperidyl)-3-methyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Degrader 36)

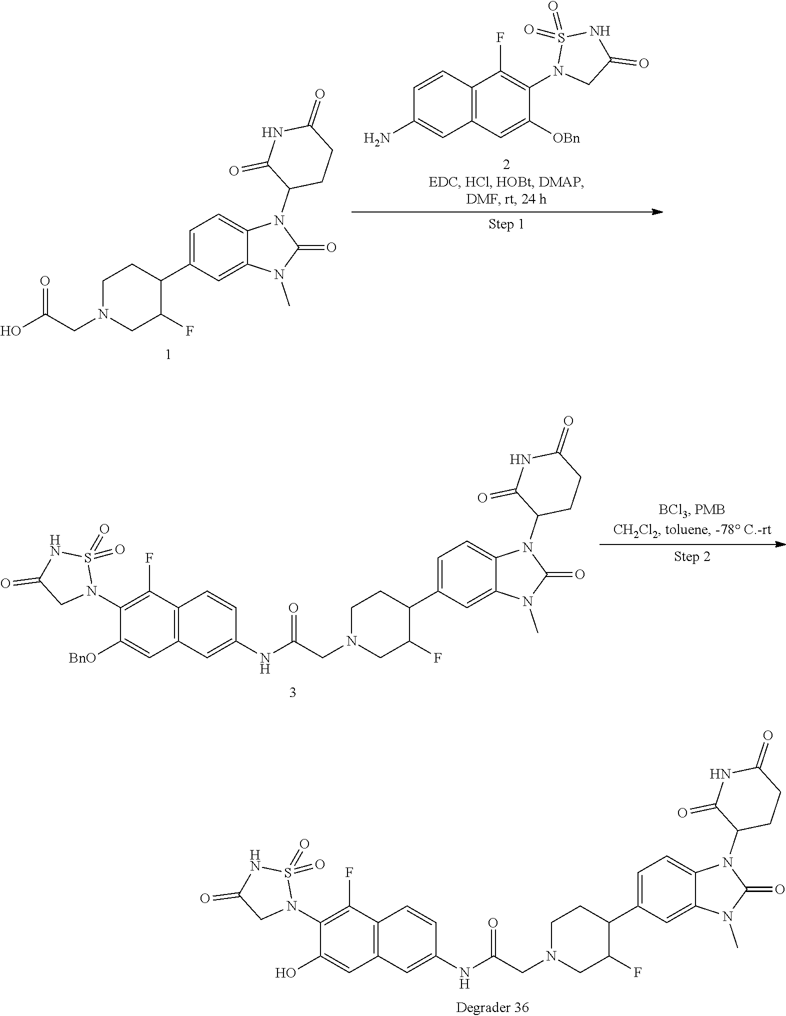 [SMILES: Cn1c(=O)n(C2CCC(=O)NC2=O)c2ccc(C3CCN(CC(=O)O)CC3F)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(C3CCN(CC(=O)Nc4ccc5c(F)c(N6CC(=O)NS6(=O)=O)c(OCc6ccccc6)cc5c4)CC3F)cc21 ; Cn1c(=O)n(C2CCC(=O)NC2=O)c2ccc(C3CCN(CC(=O)Nc4ccc5c(F)c(N6CC(=O)NS6(=O)=O)c(O)cc5c4)CC3F)cc21]

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3-fluoro-1-piperidyl]acetamide (3)

Into a 50 mL single-neck round-bottom flask containing a well-stirred solution of 5-(6-amino-3-benzyloxy-1-fluoro-2-naphthyl)-1,1-dioxo-1,2,5-thiadiazolidin-3-one (2, 111.10 mg, 201.97 μmol, TFA salt), 2-[4-[1-(2,6-dioxo-3-piperidyl)-3-methyl-2-oxo-benzimidazol-5-yl]-3-fluoro-1-piperidyl]acetic acid (1, 110 mg, 201.97 μmol, TFA salt) in anhydrous DMF (5 mL) were added EDC·HCl (116.15 mg, 605.90 μmol), HOBT (54.58 mg, 403.94 μmol) and DMAP (148.04 mg, 1.21 mmol) at room temperature. The reaction mixture was stirred at room temperature. After 24 h, the solvent was removed from the reaction mixture and the residue was treated with 1.5 N aqueous HCl (70 mL). The precipitate was filtered and dried under reduced pressure to obtain N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3-fluoro-1-piperidyl]acetamide (3, 130 mg, 135.03 μmol, 67% yield, HCl salt) as an off-white solid.

LCMS (ES+): m/z 802.0 [M+H]⁺

### Step 2: 2-[4-[1-(2,6-dioxo-3-piperidyl)-3-methyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Degrader 36)

Into a 50 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3-fluoro-1-piperidyl]acetamide (3, 140 mg, 145.42 μmol, HCl salt) and pentamethylbenzene (107.79 mg, 727.08 μmol) in anhydrous DCM (2 mL) and toluene (2 mL) was added a 1.0 M solution of boron trichloride DCM (2.91 mmol, 2.91 mL) at −78° C. Then the reaction mixture was stirred at room temperature for 2 h. The reaction mixture was quenched with 5% MeOH in DCM (5 mL) at −78° C. and excess of solvents were removed under reduced pressure. The residue was washed with MTBE (50 mL) and the crude compound was purified by reverse phase column chromatography [Purification method: Silicycle C18 column; Mobile phase A: 0.1% TFA in water; Mobile phase B: MeCN] to get 2-[4-[1-(2,6-dioxo-3-piperidyl)-3-methyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Degrader 36, 100 mg, 117.50 μmol, 81% yield, TFA salt) as an off-white solid.

LCMS (ES+): m/z 712.0 [M+H]⁺

¹H-NMR (400 MHz, DMSO-d6): δ 11.13 (s, 1H), 10.74 (s, 1H), 10.18 (s, 1H), 8.14 (s, 1H), 7.93 (d, J=9.20 Hz, 1H), 7.51 (dd, J=1.20, 9.20 Hz, 1H), 7.14-7.12 (m, 2H), 7.02-7.00 (m, 2H), 5.41-5.37 (m, 1H), 5.19-5.08 (m, 1H), 4.25-4.15 (m, 3H), 3.84-3.75 (m, 2H), 3.76-3.43 (m, 1H), 3.38 (s, 3H), 3.25-3.12 (m, 1H), 2.97-2.88 (m, 3H), 2.77-2.70 (m, 2H), 2.09-2.01 (m, 4H).

### 2-[(3R,4R)-4-[1-(2,6-dioxo-3-piperidyl)-3-methyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Degrader 37)

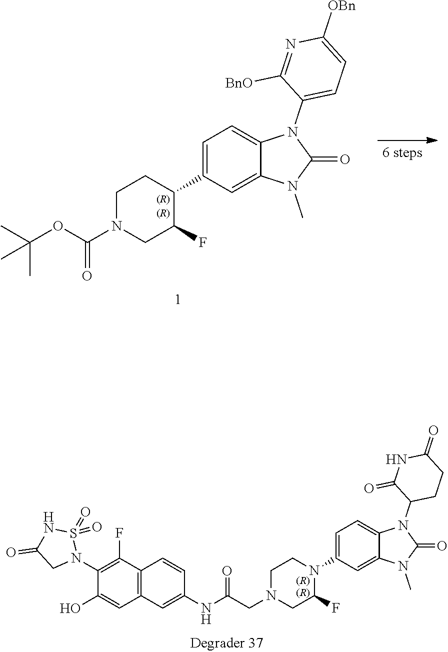

2-[(3R,4R)-4-[1-(2,6-dioxo-3-piperidyl)-3-methyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Degrader 37, 28 mg, 32.73 μmol, TFA salt) was synthesized following the same six step procedure as 2-[4-[1-(2,6-dioxo-3-piperidyl)-3-methyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Degrader 36).

LCMS (ES+): m/z 712.0 [M+H]⁺

¹HNMR (400 MHz, DMSO-d6): δ 11.12 (s, 1H), 10.61 (bs, 1H), 10.03 (s, 1H), 8.14 (s, 1H), 7.91 (d, J=8.80 Hz, 1H), 7.51 (dd, J=1.60, 9.00 Hz, 1H), 7.16-7.11 (m, 2H), 7.02-6.99 (m, 2H), 5.40-5.36 (m, 1H), 5.15-5.04 (m, 1H), 4.17 (s, 2H), 4.05-3.05 (m, 2H), 3.85-3.65 (m, 1H), 2.96-2.88 (m, 3H), 2.73-2.61 (m, 4H), 2.18-1.98 (m, 4H).

### 2-[(3S,4S)-4-[1-(2,6-dioxo-3-piperidyl)-3-methyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Degrader 38)

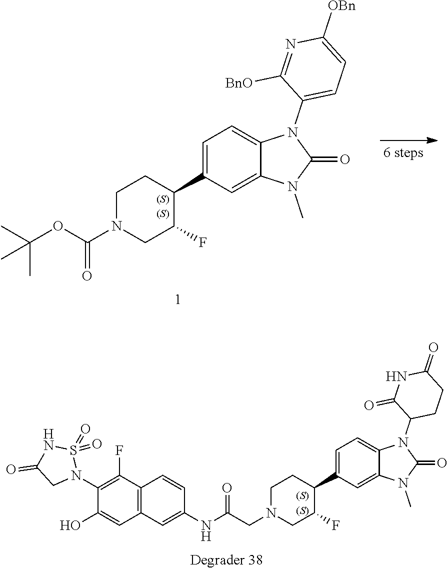

2-[(3S,4S)-4-[1-(2,6-dioxo-3-piperidyl)-3-methyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Degrader 38, 80 mg, 95.89 μmol, TFA salt) was synthesized following the same six step procedure as 2-[4-[1-(2,6-dioxo-3-piperidyl)-3-methyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Degrader 36).

LCMS (ES+): m/z 712.0 [M+H]⁺

¹HNMR (400 MHz, DMSO-d6): δ 11.11 (s, 1H), 10.71 (bs, 1H), 10.13 (s, 1H), 8.14 (s, 1H), 7.92 (d, J=8.80 Hz, 1H), 7.52 (dd, J=1.60, 9.00 Hz, 1H), 7.15-7.12 (m, 2H), 7.02-7.00 (m, 2H), 5.41-5.36 (m, 1H), 5.20-5.07 (m, 1H), 4.24-4.17 (m, 4H), 3.89-3.78 (m, 2H), 3.38 (s, 3H), 3.20-2.88 (m, 5H), 2.78-2.62 (m, 3H), 2.12-1.98 (m, 3H).

### 2-[4-[3-(2,6-dioxo-3-piperidyl)-1-methyl-indazol-6-yl]oxyphenyl]-N-[5-fluoro-7-hydroxy-6-(1,1,4-trioxo-1,2,5-thiadiazolidin-2-yl)-2-naphthyl]acetamide (Degrader 39)

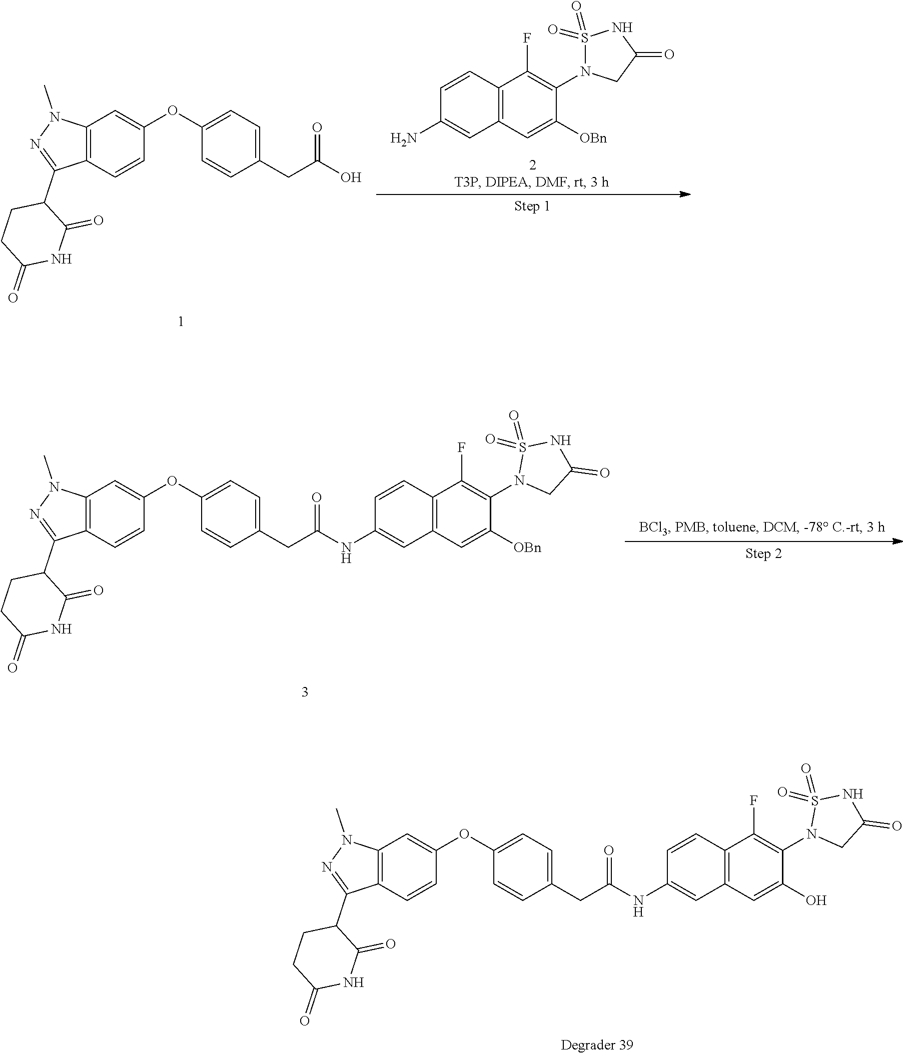 [SMILES: Cn1nc(C2CCC(=O)NC2=O)c2ccc(Oc3ccc(CC(=O)O)cc3)cc21 ; Nc1ccc2c(F)c(N3CC(=O)NS3(=O)=O)c(OCc3ccccc3)cc2c1 ; Cn1nc(C2CCC(=O)NC2=O)c2ccc(Oc3ccc(CC(=O)Nc4ccc5c(F)c(N6CC(=O)NS6(=O)=O)c(OCc6ccccc6)cc5c4)cc3)cc21 ; Cn1nc(C2CCC(=O)NC2=O)c2ccc(Oc3ccc(CC(=O)Nc4ccc5c(F)c(N6CC(=O)NS6(=O)=O)c(O)cc5c4)cc3)cc21]

### Step 1: N-[7-benzyloxy-5-fluoro-6-(1,1,4-trioxo-1,2,5-thiadiazolidin-2-yl)-2-naphthyl]-2-[4-[3-(2,6-dioxo-3-piperidyl)-1-methyl-indazol-6-yl]oxyphenyl]acetamide (3)

Into a 25 mL round-bottom flask containing a well-stirred solution of 2-[4-[3-(2,6-dioxo-3-piperidyl)-1-methyl-indazol-6-yl]oxyphenyl]acetic acid (1, 150 mg, 349.46 μmol) in DMF (5 mL) were added DIPEA (135.49 mg, 1.05 mmol, 182.60 μL) and propylphosphonic anhydride solution 50 wt. % in EtOAc (222.38 mg, 349.46 μmol, 50% purity). After stirring the mixture at room temperature for 30 min, 5-(6-amino-3-benzyloxy-1-fluoro-2-naphthyl)-1,1-dioxo-1,2,5-thiadiazolidin-3-one (2, 187.45 mg, 349.46 μmol, TFA salt) was added. The reaction mixture was stirred at ambient temperature 3 h. The volatiles were distilled off under reduced pressure and the residue was suspended in water (5 mL). The precipitate was filtered and washed with water and dried to afford N-[7-benzyloxy-5-fluoro-6-(1,1,4-trioxo-1,2,5-thiadiazolidin-2-yl)-2-naphthyl]-2-[4-[3-(2,6-dioxo-3-piperidyl)-1-methyl-indazol-6-yl]oxyphenyl]acetamide (3, 320 mg, 277.99 μmol, 80% yield) as a brown solid.

LCMS (ES−): m/z 775.0 [M−H]⁻

### Step 2: 2-[4-[3-(2,6-dioxo-3-piperidyl)-1-methyl-indazol-6-yl]oxyphenyl]-N-[5-fluoro-7-hydroxy-6-(1,1,4-trioxo-1,2,5-thiadiazolidin-2-yl)-2-naphthyl]acetamide (Degrader 39)

Into a 25 mL single neck round-bottom flask containing a well-stirred solution of N-[7-benzyloxy-5-fluoro-6-(1,1,4-trioxo-1,2,5-thiadiazolidin-2-yl)-2-naphthyl]-2-[4-[3-(2,6-dioxo-3-piperidyl)-1-methyl-indazol-6-yl]oxyphenyl]acetamide (3, 320 mg, 277.99 μmol) and pentamethylbenzene, (206.05 mg, 1.39 mmol) in anhydrous DCM (2 mL) and toluene (2 mL) were added a 1.0 M solution of BCl₃ in DCM (2.78 mmol, 2.78 mL) at −78° C. The reaction mixture was stirred at room temperature. After 3 h, the reaction mixture was quenched with 5% MeOH in DCM (5 mL) at −78° C. The volatiles were removed under reduced pressure to get the crude compound, which was purified by reverse phase column chromatography [Silicycle C18 column, Mobile phase A: 0.1% TFA in water; Mobile phase B: MeCN] to obtain 2-[4-[3-(2,6-dioxo-3-piperidyl)-1-methyl-indazol-6-yl]oxyphenyl]-N-[5-fluoro-7-hydroxy-6-(1,1,4-trioxo-1,2,5-thiadiazolidin-2-yl)-2-naphthyl]acetamide (Degrader 39, 70 mg, 96.23 μmol, 35% yield) as an off-white solid.

LCMS (ES+): m/z 687.0 [M+H]⁺

¹HNMR (400 MHz, DMSO-d6): δ 10.89 (s, 1H), 10.51 (bs, 1H), 10.43 (s, 1H), 8.19 (s, 1H), 7.88 (d, J=9.20 Hz, 1H), 7.73 (d, J=8.80 Hz, 1H), 7.47 (dd, J=2.00, 9.00 Hz, 1H), 7.40 (d, J=16.00 Hz, 2H), 7.22 (d, J=1.60 Hz, 1H), 7.03 (d, J=6.80 Hz, 2H), 6.97 (s, 1H), 6.85 (dd, J=2.00, 8.80 Hz, 1H), 4.46 (s, 2H), 4.39-4.35 (m, 1H), 3.92 (s, 3H), 3.71 (s, 2H), 2.66-2.61 (m, 2H), 2.46-2.34 (m, 1H), 2.23-2.16 (m, 1H).

### 2-[4-[1-(2,6-dioxo-3-piperidyl)-4-fluoro-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 40)

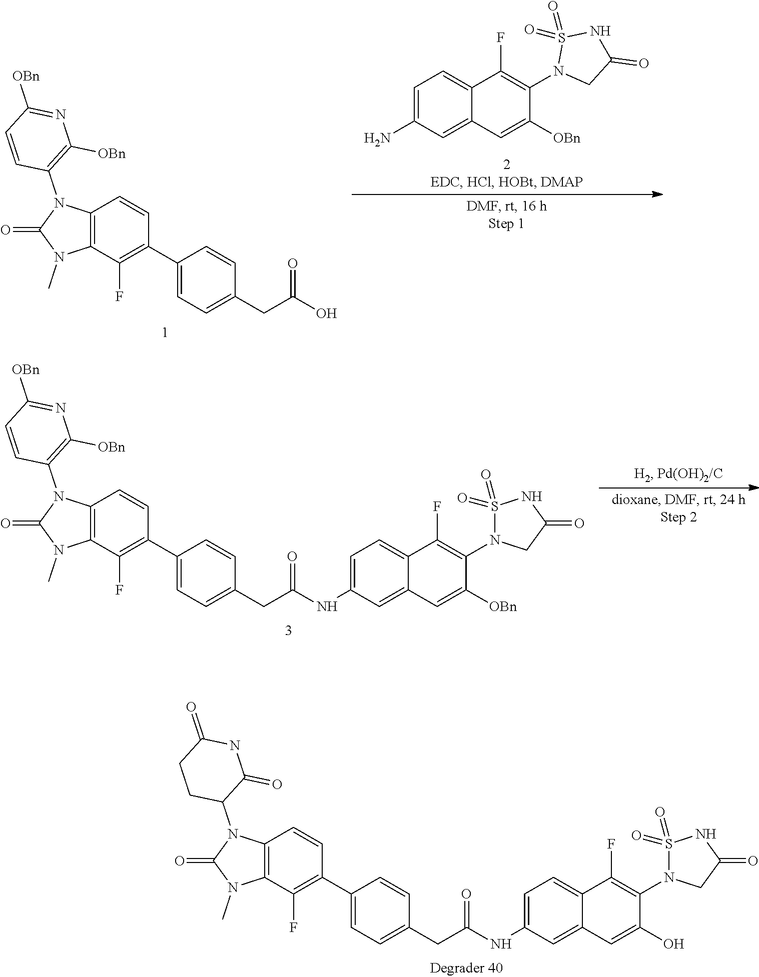 [SMILES: Cn1c(=O)n(-c2ccc(OCc3ccccc3)nc2OCc2ccccc2)c2ccc(-c3ccc(CC(=O)O)cc3)c(F)c21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(-c3ccc(CC(=O)Nc4ccc5c(F)c(N6CC(=O)NS6(=O)=O)c(OCc6ccccc6)cc5c4)cc3)c(F)c21 ; Cn1c(=O)n(C2CCC(=O)NC2=O)c2ccc(-c3ccc(CC(=O)Nc4ccc5c(F)c(N6CC(=O)NS6(=O)=O)c(O)cc5c4)cc3)c(F)c21]

### Step 1: N-[7-benzyloxy-5-fluoro-6-(1,1,4-trioxo-1,2,5-thiadiazolidin-2-yl)-2-naphthyl]-2-[4-[1-(2,6-dibenzyloxy-3-pyridyl)-4-fluoro-3-methyl-2-oxo-benzimidazol-5-yl]phenyl]acetamide (3)

Into a 8 mL screw-capped vial containing a well-stirred solution of 2-[4-[1-(2,6-dibenzyloxy-3-pyridyl)-4-fluoro-3-methyl-2-oxo-benzimidazol-5-yl]phenyl]acetic acid (1, 200 mg, 288.33 μmol) and 5-(6-amino-3-benzyloxy-1-fluoro-2-naphthyl)-1,1-dioxo-1,2,5-thiadiazolidin-3-one (2, 121.83 mg, 224.54 mmol) in DMF (2.5 mL) were sequentially added EDC (165.82 mg, 864.98 μmol), HOBT (77.92 mg, 576.65 μmol) and DMAP (176.12 mg, 1.44 mmol) at room temperature and the resulting mixture was stirred at room temperature for 16 h. The reaction mixture was concentrated under reduced pressure and treated with ice-cold water to precipitate a solid that was filtered and dried over reduced pressure to afford N-[7-benzyloxy-5-fluoro-6-(1,1,4-trioxo-1,2,5-thiadiazolidin-2-yl)-2-naphthyl]-2-[4-[1-(2,6-dibenzyloxy-3-pyridyl)-4-fluoro-3-methyl-2-oxo-benzimidazol-5-yl]phenyl]acetamide (3, 210 mg, 179.61 μmol, 62% yield) as an off-white solid.

LCMS (Es): m/z 973.1 [M+H]⁺

### Step 2: 2-[4-[1-(2,6-dioxo-3-piperidyl)-4-fluoro-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 40)

Into a 100 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[4-[1-(2,6-dibenzyloxy-3-pyridyl)-4-fluoro-3-methyl-2-oxo-benzimidazol-5-yl]phenyl]acetamide (3, 200 mg, 171.06 μmol) in 1,4-dioxane (5 mL) and DMF (2 mL) were added palladium hydroxide on carbon (20 wt. % 50% water, 240.22 mg, 342.11 μmol) at room temperature. The resultant reaction mixture was stirred under a hydrogen gas bladder for 24 h. The reaction mixture was filtered through Celite and washed successively with 1,4-dioxane (100 mL) and DMF (20 mL). The filtrate was evaporated under reduced pressure to get the crude material that was purified by reverse-phase preparative HPLC [Column: X SELECT C18 (150×19) mm, 5 μm; Mobile Phase A: 0.1% Formic acid in water and Mobile Phase B: CH₃CN] to afford 2-[4-[1-(2,6-dioxo-3-piperidyl)-4-fluoro-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 40, 26 mg, 32.53 μmol, 19% yield) as a light pink solid.

LCMS (ES+): m/z 704.8 [M+H]⁺

¹H-NMR (400 MHz, DMSO-d6): δ 11.15 (s, 1H), 10.47 (s, 1H), 10.33 (brs, 1H), 8.19 (s, 1H), 7.87 (d, J=8.80 Hz, 1H), 7.52-7.36 (m, 5H), 7.17-7.08 (m, 2H), 6.95 (s, 1H), 5.43 (dd, J=5.20, 12.80 Hz, 1H), 4.35 (s, 2H), 3.77 (s, 2H), 3.53 (s, 3H), 2.91-2.85 (m, 1H), 2.78-2.62 (m, 3H), 2.09-2.05 (m, 1H).

### 2-[4-[[3-(2,6-dioxo-3-piperidyl)-1-methyl-indazol-6-yl]amino]-3-fluoro-phenyl]-N-[5-fluoro-7-hydroxy-6-(1,1,4-trioxo-1,2,5-thiadiazolidin-2-yl)-2-naphthyl]acetamide (Degrader 41)

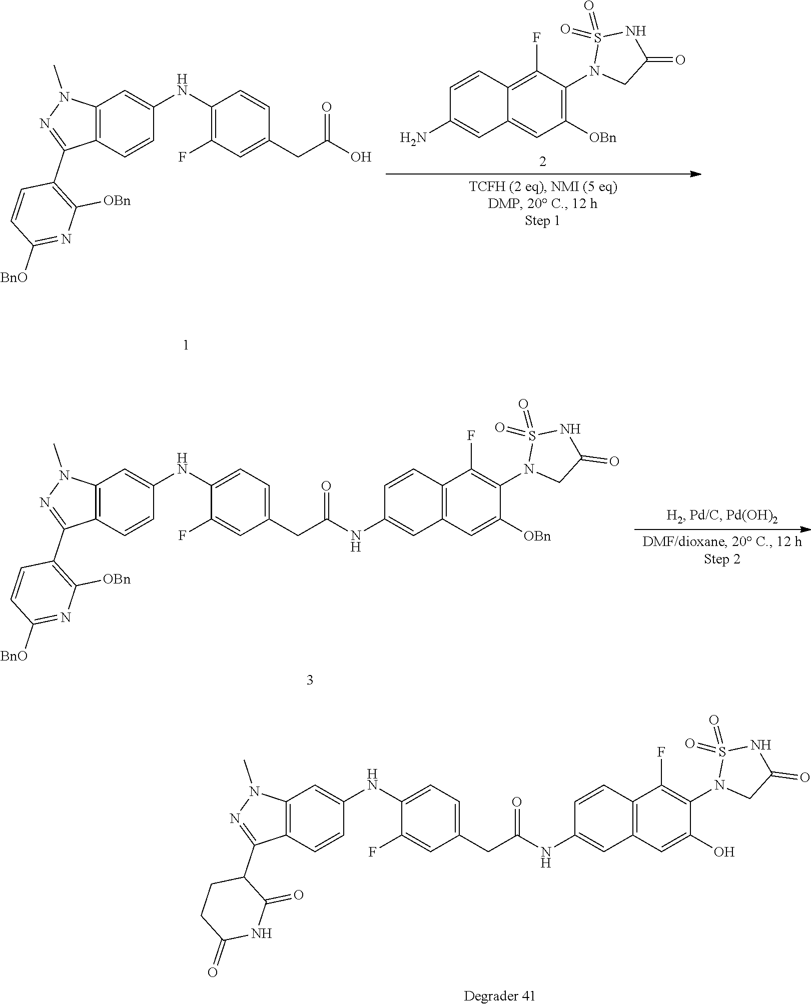 [SMILES: Cn1nc(-c2ccc(OCc3ccccc3)nc2OCc2ccccc2)c2ccc(Nc3ccc(CC(=O)O)cc3F)cc21 ; Nc1ccc2c(F)c(N3CC(=O)NS3(=O)=O)c(OCc3ccccc3)cc2c1 ; Cn1nc(-c2ccc(OCc3ccccc3)nc2OCc2ccccc2)c2ccc(Nc3ccc(CC(=O)Nc4ccc5c(F)c(N6CC(=O)NS6(=O)=O)c(OCc6ccccc6)cc5c4)cc3F)cc21 ; Cn1nc(C2CCC(=O)NC2=O)c2ccc(Nc3ccc(CC(=O)Nc4ccc5c(F)c(N6CC(=O)NS6(=O)=O)c(O)cc5c4)cc3F)cc21]

### Step 1: N-(7-(benzyloxy)-6-(1,1-dioxido-4-oxo-1,2,5-thiadiazolidin-2-yl)-5-fluoronaphthalen-2-yl)-2-(4-((3-(2,6-bis(benzyloxy)pyridin-3-yl)-1-methyl-1H-indazol-6-yl)amino)-3-fluorophenyl)acetamide (3)

To a solution of 2-(4-((3-(2,6-bis(benzyloxy)pyridin-3-yl)-1-methyl-1H-indazol-6-yl)amino)-3-fluorophenyl)acetic acid (1, 400 mg, 679.55 μmol, 1 eq) and 5-(6-amino-3-(benzyloxy)-1-fluoronaphthalen-2-yl)-1,2,5-thiadiazolidin-3-one 1,1-dioxide (2, 272.78 mg, 679.55 μmol, 1 eq) in DMF (10 mL) were added NMI (167.38 mg, 2.04 mmol, 3 eq) and TCFH (286 mg, 1.02 mmol, 1.5 eq) at 0° C. After stirring at 20° C. for 12 h, the reaction mixture was filtered and purified by reversed-phase column (neutral condition) to afford N-(7-(benzyloxy)-6-(1,1-dioxido-4-oxo-1,2,5-thiadiazolidin-2-yl)-5-fluoronaphthalen-2-yl)-2-(4-((3-(2,6-bis(benzyloxy)pyridin-3-yl)-1-methyl-1H-indazol-6-yl)amino)-3-fluorophenyl)acetamide (3, 390 mg, 401.23 μmol, 59% yield) as a light green solid.

LCMS (ESI): m/z 972.4 [M+H]⁺

### Step 2: 2-[4-[[3-(2,6-dioxo-3-piperidyl)-1-methyl-indazol-6-yl]amino]-3-fluoro-phenyl]-N-[5-fluoro-7-hydroxy-6-(1,1,4-trioxo-1,2,5-thiadiazolidin-2-yl)-2-naphthyl]acetamide (Degrader 41)

N-(7-(benzyloxy)-6-(1,1-dioxido-4-oxo-1,2,5-thiadiazolidin-2-yl)-5-fluoronaphthalen-2-yl)-2-(4-((3-(2,6-bis(benzyloxy)pyridin-3-yl)-1-methyl-1H-indazol-6-yl)amino)-3-fluorophenyl)acetamide (3, 50 mg, 51.44 μmol, 1 eq) was dissolved in 1,4-dioxane (0.2 mL) and DMF (2 mL), then put under nitrogen atmosphere using a cycle of vacuums and nitrogen purges. Pd/C (25 mg, 10% purity) and Pd(OH)₂/C (25 mg, 10% purity) were added to the solution, and the reaction mixture was submitted to a 1 atm H₂ atmosphere using a cycle of vacuums and H₂ purges from a rubber balloon. After stirring at 20° C. for 12 h under H₂ (15 psi), the reaction mixture was filtered and the filtration was purified by prep-HPLC (flow: 25 mL/min; gradient: from 62-62% water (0.1% TFA) in MeCN over 7 min; column: 3_Phenomenex Luna C18 75×30 mm×3 μm) to afford 2-[4-[[3-(2,6-dioxo-3-piperidyl)-1-methyl-indazol-6-yl]amino]-3-fluoro-phenyl]-N-[5-fluoro-7-hydroxy-6-(1,1,4-trioxo-1,2,5-thiadiazolidin-2-yl)-2-naphthyl]acetamide (Degrader 41, 14.04 mg, 19.75 μmol, 38% yield) as a yellow solid.

LCMS (ESI): m/z 704.3 [M+H]⁺

¹H NMR (400 MHz, DMSO-d₆) δ=10.88 (s, 1H), 10.43 (s, 2H), 8.22-8.10 (m, 2H), 7.87 (d, J=8.4 Hz, 1H), 7.53 (d, J=8.6 Hz, 1H), 7.46 (d, J=9.2 Hz, 1H), 7.39 (t, J=8.4 Hz, 1H), 7.26 (dd, J=1.6, 12.4 Hz, 1H), 7.12 (dd, J=1.2, 8.4 Hz, 1H), 6.96 (s, 1H), 6.92-6.84 (m, 2H), 4.41-4.24 (m, 3H), 3.83 (s, 3H), 3.69 (s, 2H), 2.66-2.60 (m, 2H), 2.32-2.25 (m, 1H), 2.22-2.11 (m, 1H).

N-(6-(1,1-dioxido-4-oxo-1,2,5-thiadiazolidin-2-yl)-5-fluoro-7-hydroxynaphthalen-2-yl)-2-(4-((1-(2,6-dioxopiperidin-3-yl)-3-methyl-2-oxo-2,3-dihydro-1H-benzo[d]imidazol-5-yl)amino)-1H-pyrazol-1-yl)acetamide (Degrader 42)

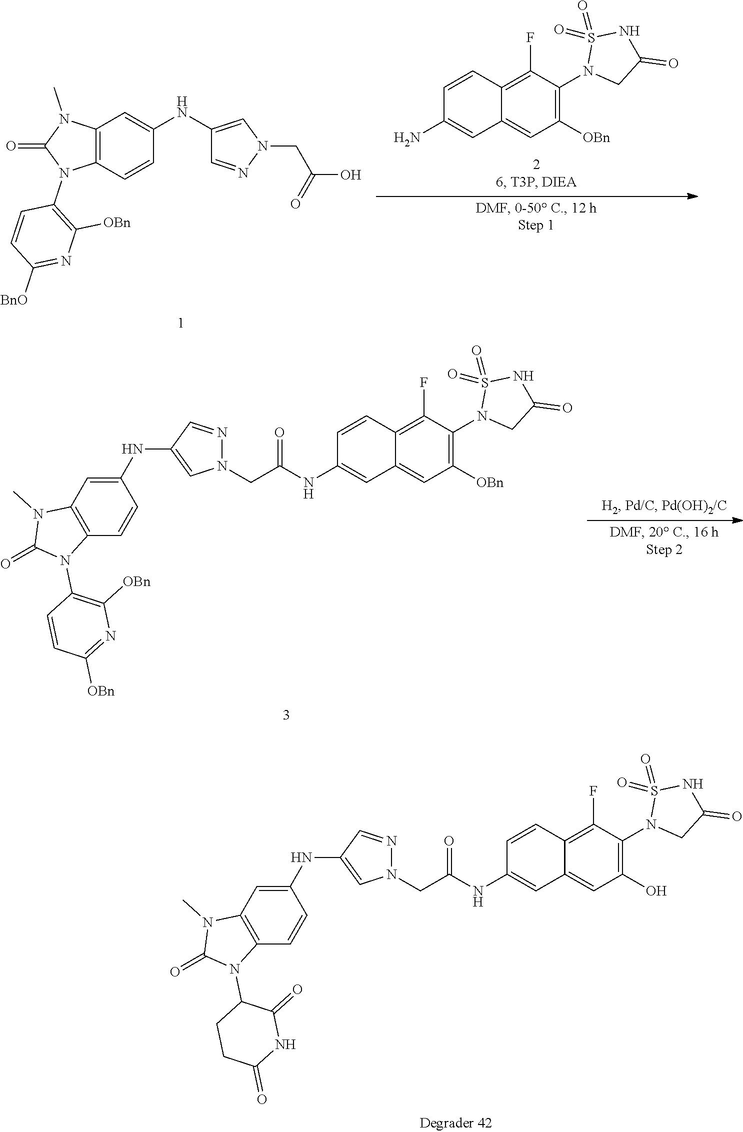 [SMILES: Cn1c(=O)n(-c2ccc(OCc3ccccc3)nc2OCc2ccccc2)c2ccc(Nc3cnn(CC(=O)O)c3)cc21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(Nc3cnn(CC(=O)Nc4ccc5c(F)c(N6CC(=O)NS6(=O)=O)c(OCc6ccccc6)cc5c4)c3)cc21 ; Cn1c(=O)n(C2CCC(=O)NC2=O)c2ccc(Nc3cnn(CC(=O)Nc4ccc5c(F)c(N6CC(=O)NS6(=O)=O)c(O)cc5c4)c3)cc21]

### Step 1: N-(7-(benzyloxy)-6-(1,1-dioxido-4-oxo-1,2,5-thiadiazolidin-2-yl)-5-fluoronaphthalen-2-yl)-2-(4-((1-(2,6-bis(benzyloxy)pyridin-3-yl)-3-methyl-2-oxo-2,3-dihydro-1H-benzo[d]imidazol-5-yl)amino)-1H-pyrazol-1-yl)acetamide (3)

To a solution of 2-(4-((1-(2,6-bis(benzyloxy)pyridin-3-yl)-3-methyl-2-oxo-2,3-dihydro-1H-benzo[d]imidazol-5-yl)amino)-1H-pyrazol-1-yl)acetic acid (1, 0.15 g, 260.15 μmol) and 5-(6-amino-3-(benzyloxy)-1-fluoronaphthalen-2-yl)-1,2,5-thiadiazolidin-3-one 1,1-dioxide (2, 104.43 mg, 260.15 μmol) in DMF (3 mL) was added N-ethyl-N-isopropyl-propan-2-amine (336.22 mg, 2.60 mmol, 453.13 μL) and T₃P (50 wt % in EtOAc) (413.87 mg, 1.30 mmol) at 0° C., then the mixture was stirred at 50° C. for 12 hrs. No workup, the mixture was used purification. The residue was purified by prep-HPLC (Column: Waters Xbridge 150*25 mm*5 um; Condition: water (0.05% ammonia hydroxide v/v)-ACN; B %: 25%-55%, 7 min) and lyophilized to give N-(7-(benzyloxy)-6-(1,1-dioxido-4-oxo-1,2,5-thiadiazolidin-2-yl)-5-fluoronaphthalen-2-yl)-2-(4-((1-(2,6-bis(benzyloxy)pyridin-3-yl)-3-methyl-2-oxo-2,3-dihydro-1H-benzo[d]imidazol-5-yl)amino)-1H-pyrazol-1-yl)acetamide (3, 0.1 g, 104.17 μmol, 40% yield) as a white solid.

LCMS (ESI): m/z 960.6 [M+H]⁺

### Step 2: N-(6-(1,1-dioxido-4-oxo-1,2,5-thiadiazolidin-2-yl)-5-fluoro-7-hydroxynaphthalen-2-yl)-2-(4-((1-(2,6-dioxopiperidin-3-yl)-3-methyl-2-oxo-2,3-dihydro-1H-benzo[d]imidazol-5-yl)amino)-1H-pyrazol-1-yl)acetamide (Degrader 42)

To a solution of N-(7-(benzyloxy)-6-(1,1-dioxido-4-oxo-1,2,5-thiadiazolidin-2-yl)-5-fluoronaphthalen-2-yl)-2-(4-((1-(2,6-bis(benzyloxy)pyridin-3-yl)-3-methyl-2-oxo-2,3-dihydro-1H-benzo[d]imidazol-5-yl)amino)-1H-pyrazol-1-yl)acetamide (3, 0.1 g, 104.17 μmol) in DMF (2 mL) was added Pd/C (50.00 mg, 41.17 μmol, 10% purity) and dihydroxypalladium (50.00 mg, 35.60 μmol, 10% purity) under H₂ atmosphere, The suspension was degassed and purged with H₂ 3 times. The mixture was stirred at 30° C. for 12 hrs under H₂ atmosphere. The mixture was filtered and concentrated in vacuum. The residue was purified by prep-HPLC (flow: 25 mL/min; gradient: from 18-48% MeCN in water (0.1% TFA) over 7 min; column: 3_Phenomenex Luna C18 75×30 mm×3 um) to afford N-(6-(1,1-dioxido-4-oxo-1,2,5-thiadiazolidin-2-yl)-5-fluoro-7-hydroxynaphthalen-2-yl)-2-(4-((1-(2,6-dioxopiperidin-3-yl)-3-methyl-2-oxo-2,3-dihydro-1H-benzo[d]imidazol-5-yl)amino)-1H-pyrazol-1-yl)acetamide (Degrader 42, 49.48 mg, 70.82 μmol, 68% yield, TFA salt) as a gray solid.

LCMS (ESI): m/z 692.0 [M+H]⁺

¹H NMR (400 MHz, DMSO) δ 11.05 (s, 1H), 10.55 (s, 1H), 10.47 (s, 1H), 8.17 (s, 1H), 7.89 (d, J=8.9 Hz, 1H), 7.73 (s, 1H), 7.49-7.42 (m, 1H), 7.40-7.39 (m, 1H), 6.97 (s, 1H), 6.89 (d, J=8.5 Hz, 1H), 6.61 (d, J=2.2 Hz, 1H), 6.51 (dd, J=8.4, 2.2 Hz, 1H), 5.27 (dd, J=12.9, 5.4 Hz, 1H), 5.02 (s, 2H), 4.39 (d, J=11.2 Hz, 2H), 3.28 (s, 3H), 2.95-2.81 (m, 1H), 2.74-2.56 (m, 2H), 2.04-1.93 (m, 1H).

### 2-[1-[1-(2,6-dioxo-3-piperidyl)-6-fluoro-3-methyl-2-oxo-benzimidazol-5-yl]-4-piperidyl]-N-[5-fluoro-7-hydroxy-6-(1,1,4-trioxo-1,2,5-thiadiazolidin-2-yl)-2-naphthyl]acetamide (Degrader 43)

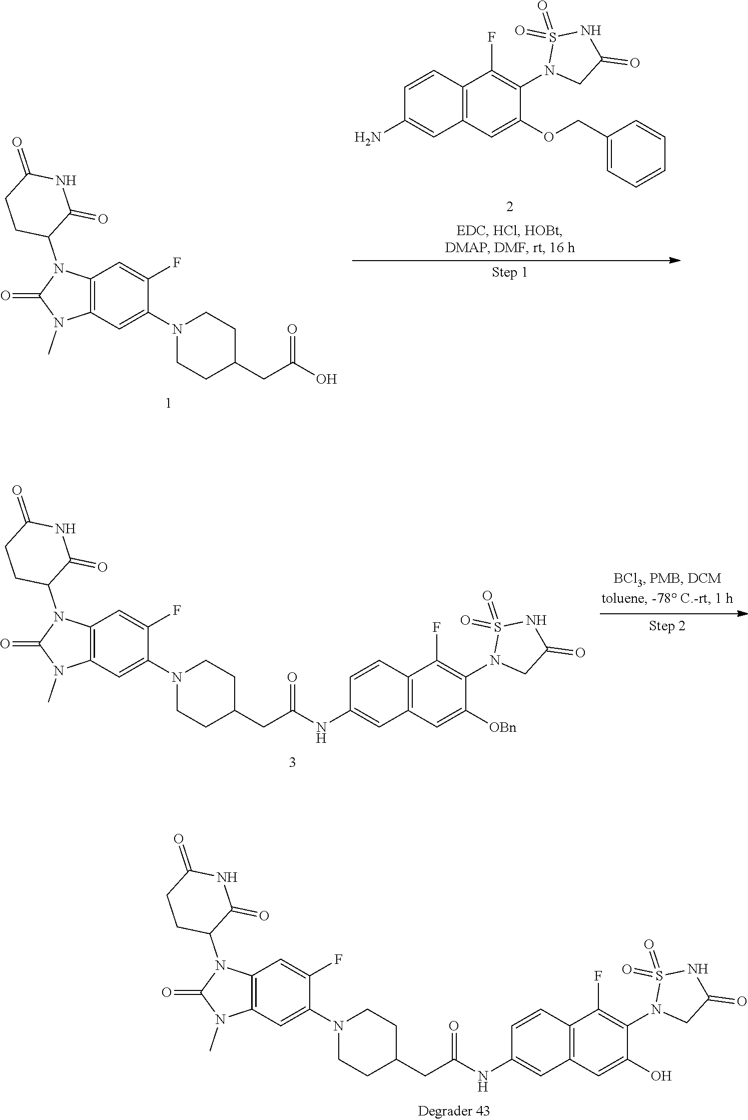 [SMILES: Cn1c(=O)n(C2CCC(=O)NC2=O)c2cc(F)c(N3CCC(CC(=O)O)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2cc(F)c(N3CCC(CC(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2cc(F)c(N3CCC(CC(=O)Nc4ccc5c(F)c(N6CC(=O)NS6(=O)=O)c(O)cc5c4)CC3)cc21]

### Step 1: N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-6-fluoro-3-methyl-2-oxo-benzimidazol-5-yl]-4-piperidyl]acetamide (3)

Into a 25 mL single-neck round-bottom flask containing a well-stirred solution of 2-[1-[1-(2,6-dioxo-3-piperidyl)-6-fluoro-3-methyl-2-oxo-benzimidazol-5-yl]-4-piperidyl]acetic acid (1, 140 mg, 262.15 μmol, TFA salt) and 5-(6-amino-3-benzyloxy-1-fluoro-2-naphthyl)-1,1-dioxo-1,2,5-thiadiazolidin-3-one (2, 158.97 mg, 262.15 μmol, TFA salt) in anhydrous DMF (2.5 mL) were added EDC·HCl (150.76 mg, 786.45 μmol), DMAP (192.16 mg, 1.57 mmol) and HOBT (70.85 mg, 524.30 μmol). After 16 h, the solvent was removed and the residue was treated with 1.5 N HCl (2 mL). The precipitate was filtered and dried under reduced pressure to get N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-6-fluoro-3-methyl-2-oxo-benzimidazol-5-yl]-4-piperidyl]acetamide (3, 185 mg, 153.60 μmol, 59% yield, HCl salt) as an off-white color solid.

LCMS (ES+): m/z 802.0 [M+H]⁺

### Step 2: 2-[1-[1-(2,6-dioxo-3-piperidyl)-6-fluoro-3-methyl-2-oxo-benzimidazol-5-yl]-4-piperidyl]-N-[5-fluoro-7-hydroxy-6-(1,1,4-trioxo-1,2,5-thiadiazolidin-2-yl)-2-naphthyl]acetamide (Degrader 43)

Into a 50 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-6-fluoro-3-methyl-2-oxo-benzimidazol-5-yl]-4-piperidyl]acetamide (3, 185 mg, 153.76 μmol, HCl salt) and pentamethylbenzene (136.76 mg, 922.53 μmol) in anhydrous DCM (3 mL) and toluene (3 mL) was added a 1.0 M solution of boron trichloride in methylene chloride (360.31 mg, 3.08 mmol, 3.08 mL) at −78° C. The reaction mixture was stirred at room temperature for 1 h. The reaction mixture quenched with 5% methanol in DCM (2 mL) at −78° C. The volatiles were removed under reduced pressure and the residue was triturated with MTBE (60 mL) and filtered. The crude compound was purified by reverse phase column chromatography [Purification method: Silicycle C18 column; Mobile phase A: 0.1% Formic acid in water; Mobile phase B: MeCN] to get 2-[1-[1-(2,6-dioxo-3-piperidyl)-6-fluoro-3-methyl-2-oxo-benzimidazol-5-yl]-4-piperidyl]-N-[5-fluoro-7-hydroxy-6-(1,1,4-trioxo-1,2,5-thiadiazolidin-2-yl)-2-naphthyl]acetamide (Degrader 43, 60 mg, 82.78 μmol, 54% yield) as a colorless solid.

LCMS (ES+): m/z 711.8 [M+H]⁺

¹HNMR (400 MHz, DMSO-d6): δ 11.06 (s, 1H), 10.18 (s, 1H), 10.12 (s, 1H), 8.20 (s, 1H), 7.85 (d, J=8.80 Hz, 1H), 7.45 (dd, J=1.60, 9.00 Hz, 1H), 7.14 (d, J=12.00 Hz, 1H), 6.99-6.97 (m, 2H), 5.33-5.29 (m, 1H), 4.28 (s, 2H), 3.33 (s, 3H), 3.29-3.26 (m, 2H), 2.95-2.56 (m, 6H), 2.46-2.40 (m, 2H), 2.01-1.90 (m, 2H), 1.90-1.80 (m, 2H), 1.55-1.39 (m, 2H).

### 2-[4-[1-(2,6-dioxo-3-piperidyl)-6-fluoro-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 44)

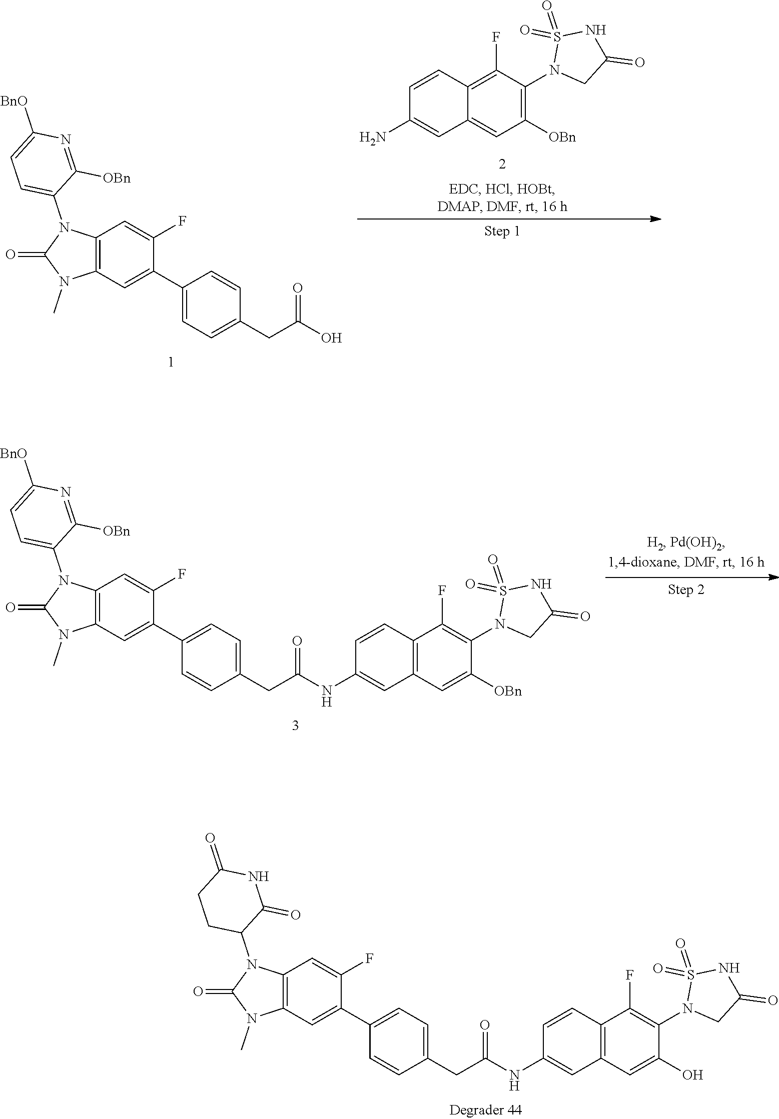 [SMILES: Cn1c(=O)n(-c2ccc(OCc3ccccc3)nc2OCc2ccccc2)c2cc(F)c(-c3ccc(CC(=O)O)cc3)cc21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2cc(F)c(-c3ccc(CC(=O)Nc4ccc5c(F)c(N6CC(=O)NS6(=O)=O)c(OCc6ccccc6)cc5c4)cc3)cc21 ; Cn1c(=O)n(C2CCC(=O)NC2=O)c2cc(F)c(-c3ccc(CC(=O)Nc4ccc5c(F)c(N6CC(=O)NS6(=O)=O)c(O)cc5c4)cc3)cc21]

### Step 1: N-[7-benzyloxy-5-fluoro-6-(1,1,4-trioxo-1,2,5-thiadiazolidin-2-yl)-2-naphthyl]-2-[4-[1-(2,6-dibenzyloxy-3-pyridyl)-6-fluoro-3-methyl-2-oxo-benzimidazol-5-yl]phenyl]acetamide (3)

Into a 50 mL single neck round bottom flask containing a well-stirred solution of 2-[4-[1-(2,6-dibenzyloxy-3-pyridyl)-6-fluoro-3-methyl-2-oxo-benzimidazol-5-yl]phenyl]acetic acid (1, 250 mg, 322.25 μmol) and 5-(6-amino-3-benzyloxy-1-fluoro-2-naphthyl)-1,1-dioxo-1,2,5-thiadiazolidin-3-one (2, 134.74 mg, 250.96 μmol, TFA salt) in anhydrous DMF (4 mL) were added EDC (185.32 mg, 966.74 μmol), DMAP (236.21 mg, 1.93 mmol) and HOBT (87.08 mg, 644.49 μmol) at room temperature. The reaction mixture was stirred at room temperature. After 16 h, the reaction mixture concentrated under reduced pressure and the residue was suspended in aqueous 1.5N HCl (3 mL) at 0° C. The precipitate was filtered, washed with cold water (4 mL) and dried to afford N-[7-benzyloxy-5-fluoro-6-(1,1,4-trioxo-1,2,5-thiadiazolidin-2-yl)-2-naphthyl]-2-[4-[1-(2,6-dibenzyloxy-3-pyridyl)-6-fluoro-3-methyl-2-oxo-benzimidazol-5-yl]phenyl]acetamide (3, 220 mg, 215.14 μmol, 67% yield) as a pale brown solid.

LCMS (ES+): m/z 973.2 [M+H]⁺

### Step 2: 2-[4-[1-(2,6-dioxo-3-piperidyl)-6-fluoro-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 44)

Into a 50 mL single neck round bottom flask containing a well-stirred solution of N-[7-benzyloxy-5-fluoro-6-(1,1,4-trioxo-1,2,5-thiadiazolidin-2-yl)-2-naphthyl]-2-[4-[1-(2,6-dibenzyloxy-3-pyridyl)-6-fluoro-3-methyl-2-oxo-benzimidazol-5-yl]phenyl]acetamide (3, 220 mg, 214.80 μmol) in 1,4-dioxane (4 mL) and DMF (2 mL) was added 20 wt. % palladium hydroxide on carbon (150.83 mg, 214.80 μmol, 20% purity). The suspension was stirred under hydrogen atmosphere at room temperature. After 16 h, the reaction mixture was filtered through Celite bed and washed with mixture of 1,4-dioxane (15 mL) and DMF (7 mL). The filtrate was concentrated under reduced pressure and the crude compound was purified by reverse phase prep HPLC [Purification method: Column: Sunfire C18, (19×150 mm), 5 micron; Mobile phase A: 0.1% TFA in MQ water; Mobile phase B: MeCN] to afford 2-[4-[1-(2,6-dioxo-3-piperidyl)-6-fluoro-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 44, 55 mg, 72.11 μmol, 34% yield) as a pale yellow solid.

LCMS (ES+): m/z 703.0 [M−H]⁻

¹H NMR (400 MHz, DMSO) δ 11.12 (s, 1H), 10.42 (s, 1H), 9.80 (s, 1H), 8.17-8.13 (m, 1H), 7.83 (d, J=9.0 Hz, 1H), 7.56-7.51 (m, 2H), 7.49-7.43 (m, 3H), 7.33 (d, J=6.7 Hz, 1H), 7.27 (d, J=11.0 Hz, 1H), 6.93 (s, 1H), 5.39 (dd, J=12.8, 5.3 Hz, 1H), 4.09 (s, 2H), 3.75 (s, 2H), 2.95-2.70 (m, 2H), 2.66-2.59 (m, 1H), 2.09-1.99 (m, 1H). Additional protons under solvent peak.

### 2-[4-[1-(2,6-dioxo-3-piperidyl)-3-(2-methoxyethyl)-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 45)

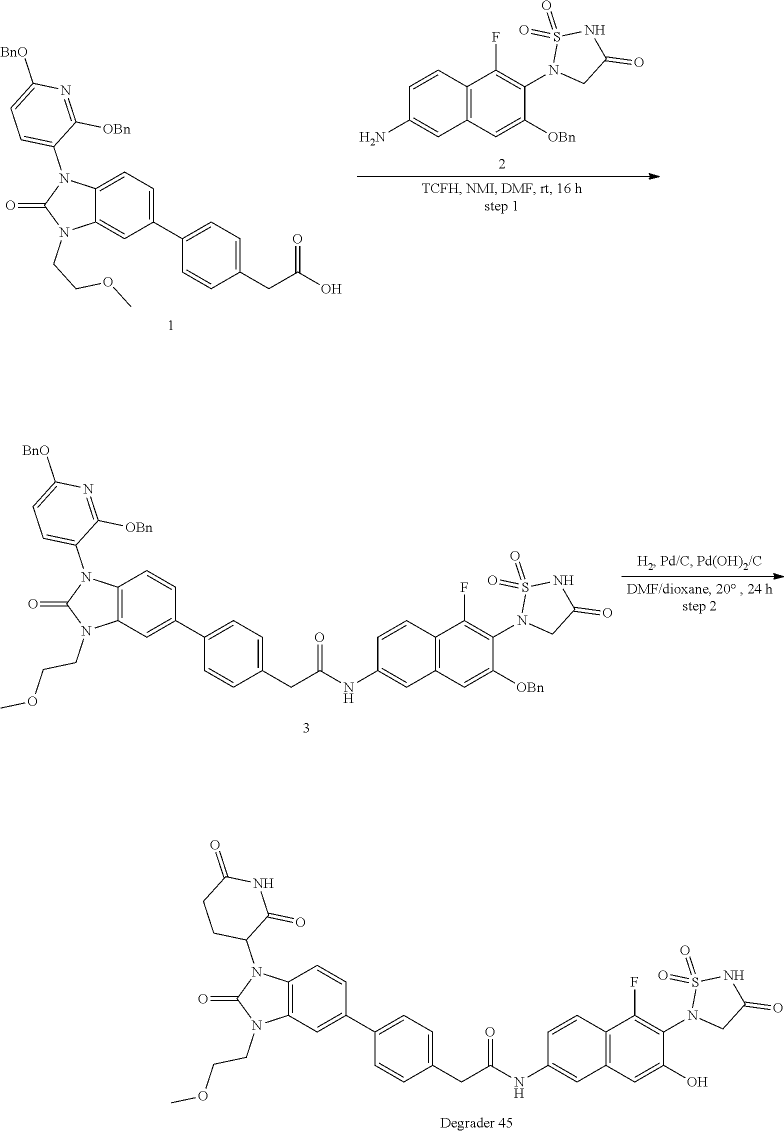 [SMILES: COCCn1c(=O)n(-c2ccc(OCc3ccccc3)nc2OCc2ccccc2)c2ccc(-c3ccc(CC(=O)O)cc3)cc21 ; Nc1ccc2c(F)c(N3CC(=O)NS3(=O)=O)c(OCc3ccccc3)cc2c1 ; COCCn1c(=O)n(-c2ccc(OCc3ccccc3)nc2OCc2ccccc2)c2ccc(-c3ccc(CC(=O)Nc4ccc5c(F)c(N6CC(=O)NS6(=O)=O)c(OCc6ccccc6)cc5c4)cc3)cc21 ; COCCn1c(=O)n(C2CCC(=O)NC2=O)c2ccc(-c3ccc(CC(=O)Nc4ccc5c(F)c(N6CC(=O)NS6(=O)=O)c(O)cc5c4)cc3)cc21]

### Step 1: N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-(2-methoxyethyl)-2-oxo-2,3-dihydro-1H-benzo[d]imidazol-5-yl)phenyl)acetamide (3)

To a solution of 5-(6-amino-3-(benzyloxy)-1-fluoronaphthalen-2-yl)-1,2,5-thiadiazolidin-3-one 1,1-dioxide (2, 253.33 mg, 631.11 μmol, 1.1 eq) and 2-(4-(1-(2,6-bis(benzyloxy)pyridin-3-yl)-3-(2-methoxyethyl)-2-oxo-2,3-dihydro-1H-benzo[d]imidazol-5-yl)phenyl)acetic acid (1, 380 mg, 567.83 μmol, 1 eq) in DMF (8 mL) was added 1-Methylimidazole (139.86 mg, 1.70 mmol, 135.78 μL, 3 eq and [chloro(dimethylamino)methylene]-dimethyl-ammonium; hexafluorophosphate (238.98 mg, 851.75 μmol, 3 eq at 0° C. The mixture was stirred at 20° C. for 12 h. The reaction mixture was concentrated under reduced to remove DMF. The residue was purified by prep-HPLC (flow: 25 mL/min; gradient: from 37-67% water (10 mM NH₄HCO₃) in MeCN over 9 min; column: Waters Xbridge C18 150×25 mm×5 μm) to give N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-(2-methoxyethyl)-2-oxo-2,3-dihydro-1H-benzo[d]imidazol-5-yl)phenyl)acetamide (3, 240 mg, 235.42 μmol, 41% yield) as an off-white solid.

LC-MS (ESI): m/z 999.3 [M+H]⁺

### Step 2: 2-[4-[1-(2,6-dioxo-3-piperidyl)-3-(2-methoxyethyl)-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 45)

N-[7-benzyloxy-5-fluoro-6-(1,1,4-trioxo-1,2,5-thiadiazolidin-2-yl)-2-naphthyl]-2-[4-[1-(2,6-dibenzyloxy-3-pyridyl)-3-(2-methoxyethyl)-2-oxo-benzimidazol-5-yl]phenyl]acetamide (3, 120 mg, 120.11 μmol, 1 eq) is dissolved in DMF (10 mL) and dioxane (1 mL), then put under nitrogen atmosphere using a cycle of vacuums and nitrogen purges. Pd/C (50 mg, 10% purity) and Pd(OH)₂ (50 mg, 10% purity) were added to the solution, and the reaction mixture was submitted to a 1 atm hydrogen atmosphere using a cycle of vacuums and hydrogen purges from a rubber balloon. The reaction mixture was stirred at 20° C. for 16 h under H₂ (15 psi). Another part of Pd/C (50 mg) was added to the mixture and it was stirred at 20° C. for additional 8 h. The reaction mixture was filtered, and the filtrate was concentrated under reduced pressure. The residue was purified by prep-HPLC (flow: 25 mL/min; gradient: from 30-60% water (0.1% TFA) in MeCN over 7 min; column: 3_Phenomenex Luna C18 75×30 mm×3 μm) and lyophilized to give 2-[4-[1-(2,6-dioxo-3-piperidyl)-3-(2-methoxyethyl)-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Degrader 45, 30.13 mg, 35.31 μmol, 29% yield, TFA salt) as a purple solid. LC-MS (ESI): m/z 731.2 [M+H]+

¹H NMR (400 MHz, DMSO-d₆) δ=11.14 (s, 1H), 10.45 (s, 1H), 10.20 (br s, 1H), 8.17 (s, 1H), 7.86 (d, J=9.0 Hz, 1H), 7.68-7.61 (m, 2H), 7.55 (d, J=1.3 Hz, 1H), 7.50-7.41 (m, 3H), 7.36-7.26 (m, 1H), 7.23-7.13 (m, 1H), 7.09 (s, 1H), 6.99-6.92 (m, 1H), 5.41 (dd, J=5.3, 12.9 Hz, 1H), 4.28 (s, 2H), 4.17-3.98 (m, 2H), 3.75 (br s, 2H), 3.64 (t, J=5.4 Hz, 2H), 3.27-3.24 (m, 3H), 2.98-2.85 (m, 1H), 2.81-2.71 (m, 1H), 2.70-2.61 (m, 1H), 2.10-2.00 (m, 1H).

### 2-[1-[1-(2,6-dioxo-3-piperidyl)-4-fluoro-3-methyl-2-oxo-benzimidazol-5-yl]-4-piperidyl]-N-[5-fluoro-7-hydroxy-6-(1,1,4-trioxo-1,2,5-thiadiazolidin-2-yl)-2-naphthyl]acetamide (Degrader 46)

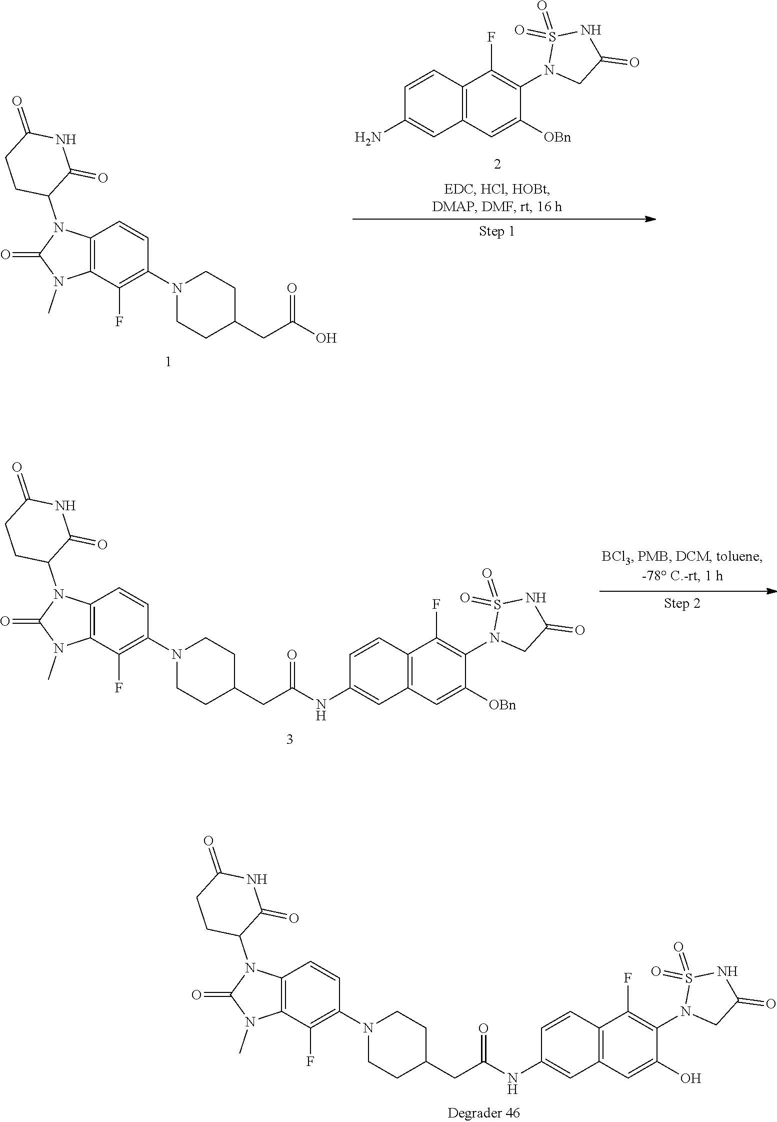 [SMILES: Cn1c(=O)n(C2CCC(=O)NC2=O)c2ccc(N3CCC(CC(=O)O)CC3)c(F)c21 ; Nc1ccc2c(F)c(N3CC(=O)NS3(=O)=O)c(OCc3ccccc3)cc2c1 ; Cn1c(=O)n(C2CCC(=O)NC2=O)c2ccc(N3CCC(CC(=O)Nc4ccc5c(F)c(N6CC(=O)NS6(=O)=O)c(OCc6ccccc6)cc5c4)CC3)c(F)c21 ; Cn1c(=O)n(C2CCC(=O)NC2=O)c2ccc(N3CCC(CC(=O)Nc4ccc5c(F)c(N6CC(=O)NS6(=O)=O)c(O)cc5c4)CC3)c(F)c21]

### Step 1: N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-4-fluoro-3-methyl-2-oxo-benzimidazol-5-yl]-4-piperidyl]acetamide (4)

Into a 10 mL single-neck round-bottom flask containing a well-stirred solution of 2-[1-[1-(2,6-dioxo-3-piperidyl)-4-fluoro-3-methyl-2-oxo-benzimidazol-5-yl]-4-piperidyl]acetic acid (1, 210 mg, 375.92 μmol, TFA salt) and 5-(6-amino-3-benzyloxy-1-fluoro-2-naphthyl)-1,1-dioxo-1,2,5-thiadiazolidin-3-one (2, 201.83 mg, 375.92 μmol, TFA salt) in anhydrous DMF (5 mL) were added EDC·HCl (216.19 mg, 1.13 mmol), DMAP (275.55 mg, 2.26 mmol) and HOBT (101.59 mg, 751.83 μmol). The reaction mixture was stirred at room temperature for 16 h. The solvent was removed under reduced pressure and the residue was treated with 1.5 N HCL. The precipitate was filtered and dried under reduced pressure to obtain N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-4-fluoro-3-methyl-2-oxo-benzimidazol-5-yl]-4-piperidyl]acetamide (3, 50 mg, 60.92 μmol, 16% yield, HCl salt) as off white solid.

LCMS (ES+): m/z 802.0 [M+H]⁺

### Step 2: 2-[1-[1-(2,6-dioxo-3-piperidyl)-4-fluoro-3-methyl-2-oxo-benzimidazol-5-yl]-4-piperidyl]-N-[5-fluoro-7-hydroxy-6-(1,1,4-trioxo-1,2,5-thiadiazolidin-2-yl)-2-naphthyl]acetamide (Degrader 46)

Into a 50 mL single-neck round-bottom flask containing a well-stirred suspension of N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-4-fluoro-3-methyl-2-oxo-benzimidazol-5-yl]-4-piperidyl]acetamide (3, 280 mg, 244.70 μmol, HCl salt) and pentamethylbenzene (217.66 mg, 1.47 mmol, 237.36 μL) in DCM (5 mL) and toluene (5 mL) was added a 1.0 M solution of boron trichloride in methylene chloride (573.44 mg, 4.89 mmol, 4.89 mL) at −78° C. The reaction mixture was stirred at room temperature for 1 h. The reaction mixture was cooled to −78° C. and was quenched with 5% methanol in DCM (3 mL) and the volatiles were removed, and the residue was triturated with methyl tertiary butyl ether (60 mL), filtered and dried. The crude compound was purified by reverse phase column chromatography [Purification method: Silicycle C18 column; Mobile phase A: 0.1% TFA in water; Mobile phase B: MeCN] to get 2-[1-[1-(2,6-dioxo-3-piperidyl)-4-fluoro-3-methyl-2-oxo-benzimidazol-5-yl]-4-piperidyl]-N-[5-fluoro-7-hydroxy-6-(1,1,4-trioxo-1,2,5-thiadiazolidin-2-yl)-2-naphthyl]acetamide (Degrader 46, 42 mg, 49.70 μmol, 20% yield, TFA salt) as an off-white solid.

LCMS (ES+): m/z 712.0 [M+H]⁺

¹HNMR (400 MHz, DMSO-d6): δ 11.11 (s, 1H), 10.18 (s, 1H), 10.05 (s, 1H), 9.82 (s, 1H), 8.19 (s, 1H), 7.84 (d, J=9.20 Hz, 1H), 7.44 (d, J=8.80 Hz, 1H), 6.96 (s, 1H), 6.86 (d, J=8.40 Hz, 1H), 6.77 (t, J=8.40 Hz, 1H), 5.36-5.31 (m, 1H), 4.22 (s, 2H), 3.26-3.17 (m, 2H), 2.89-2.72 (m, 1H), 2.69-2.60 (m, 4H), 2.39-2.34 (m, 2H), 2.02-1.94 (m, 3H), 1.84-1.76 (m, 3H), 1.60-1.30 (m, 3H).

### N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-ethyl-2-oxo-2,3-dihydro-1H-benzo[d]imidazol-5-yl)-3-fluorophenyl)acetamide (Degrader 47)

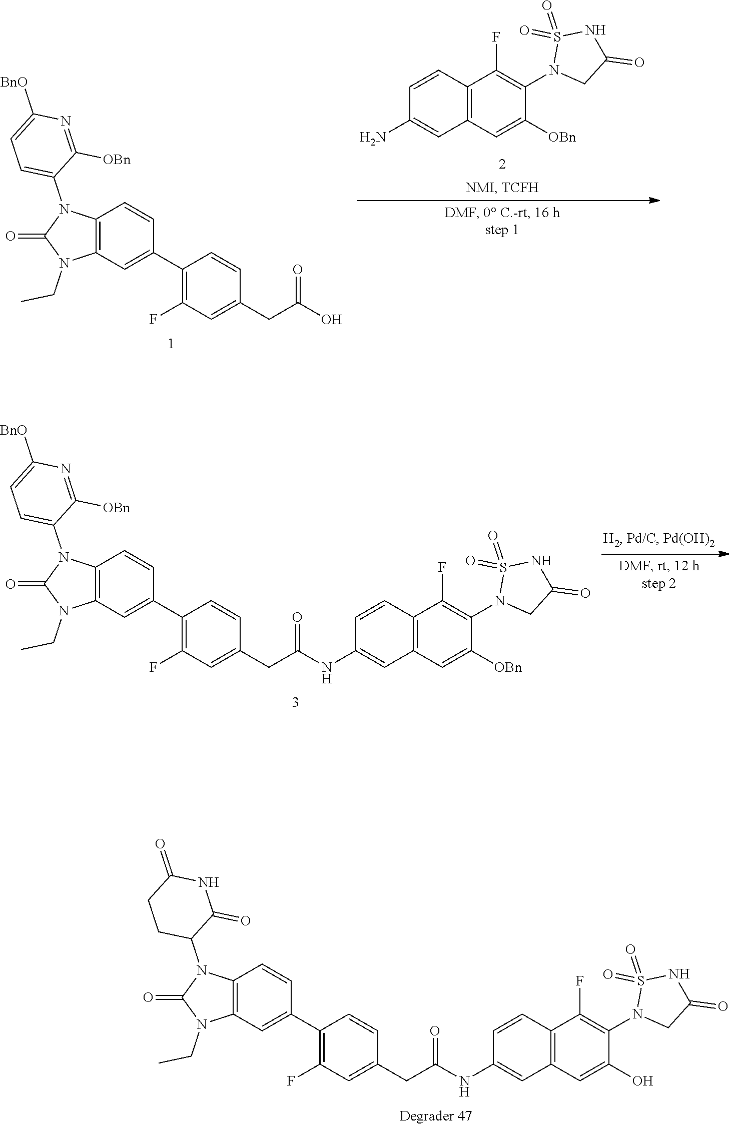 [SMILES: CCn1c(=O)n(-c2ccc(OCc3ccccc3)nc2OCc2ccccc2)c2ccc(-c3ccc(CC(=O)O)cc3F)cc21 ; Nc1ccc2c(F)c(N3CC(=O)NS3(=O)=O)c(OCc3ccccc3)cc2c1 ; CCn1c(=O)n(-c2ccc(OCc3ccccc3)nc2OCc2ccccc2)c2ccc(-c3ccc(CC(=O)Nc4ccc5c(F)c(N6CC(=O)NS6(=O)=O)c(OCc6ccccc6)cc5c4)cc3F)cc21 ; CCn1c(=O)n(C2CCC(=O)NC2=O)c2ccc(-c3ccc(CC(=O)Nc4ccc5c(F)c(N6CC(=O)NS6(=O)=O)c(O)cc5c4)cc3F)cc21]

### Step 1: N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-ethyl-2-oxo-2,3-dihydro-1H-benzo[d]imidazol-5-yl)-3-fluorophenyl)acetamide (3)

To a solution of 2-(4-(1-(2,6-bis(benzyloxy)pyridin-3-yl)-3-ethyl-2-oxo-2,3-dihydro-1H-benzo[d]imidazol-5-yl)-3-fluorophenyl)acetic acid (1, 130 mg, 215.36 μmol, 1 eq), 5-(6-amino-3-(benzyloxy)-1-fluoronaphthalen-2-yl)-1,2,5-thiadiazolidin-3-one 1,1-dioxide (2, 111.00 mg, 215.36 μmol, TFA salt, 1 eq) and NMI (88.41 mg, 1.08 mmol, 85.83 μL, 5 eq) in DMF (5 mL) was added TCFH (181.28 mg, 646.08 μmol, 3 eq) at 0° C. The mixture was stirred at 20° C. for 16 h. The mixture was concentrated under vacuum to give a residue. The residue was purified by prep-HPLC (flow: 25 mL/min; gradient: from 25-55% MeCN in water (0.05% ammonia hydroxide v/v)-ACN over 12 min; column: Waters Xbridge C18 150×25 mm×5 um) to afford N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-ethyl-2-oxo-2,3-dihydro-1H-benzo[d]imidazol-5-yl)-3-fluorophenyl)acetamide (3, 150 mg, 151.97 μmol, 70% yield) as a white solid.

LCMS (ESI): m/z 987.4 [M+H]⁺

¹H NMR (400 MHz, DMSO-d₆) δ 10.50 (s, 1H), 8.29 (s, 1H), 7.93-784 (m, 2H), 7.56-7.50 (m, 4H), 7.45 (t, J=7.2 Hz, 3H), 7.42-7.32 (m, 6H), 7.32-7.29 (m, 2H), 7.29-7.25 (m, 6H), 7.22-7.15 (m, 2H), 6.79 (d, J=8.0 Hz, 1H), 6.63 (d, J=8.4 Hz, 1H), 5.46-5.34 (m, 4H), 5.26 (s, 2H), 4.22 (s, 2H), 3.97 (q, J=7.2 Hz, 2H), 3.79 (s, 2H), 1.26 (t, J=7.2 Hz, 3H).

### Step 2: N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-ethyl-2-oxo-2,3-dihydro-1H-benzo[d]imidazol-5-yl)-3-fluorophenyl)acetamide (Degrader 47)

To a solution of N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-ethyl-2-oxo-2,3-dihydro-1H-benzo[d]imidazol-5-yl)-3-fluorophenyl)acetamide (3, 150 mg, 151.97 μmol, 1 eq) in DMF (12 mL) were added Pd/C (150 mg, 10% purity) and Pd(OH)₂/C (150 mg, 10% purity) under N₂ atmosphere. The mixture was stirred under H₂ (15 psi) at 15° C. for 12 h. The mixture was filtered and concentrated under vacuum. The residue was purified by prep-HPLC (flow: 25 mL/min; gradient: from 36-66% MeCN in water (0.1% TFA) over 12 min; column: Phenomenex Synergi C18 150×25 mm×10 um) to afford N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-ethyl-2-oxo-2,3-dihydro-1H-benzo[d]imidazol-5-yl)-3-fluorophenyl)acetamide (Degrader 47, 67.05 mg, 92.36 μmol, 61% yield) as an off-white solid.

LCMS (ESI): m/z 719.3 [M+H]⁺

¹H NMR (400 MHz, DMSO-d₆) δ 11.11 (s, 1H), 10.46 (s, 2H), 8.17 (s, 1H), 7.87 (d, J=9.2 Hz, 1H), 7.53 (t, J=8.0 Hz, 1H), 7.46 (dd, J=1.6, 9.2 Hz, 1H), 7.40 (s, 1H), 7.34-7.26 (m, 2H), 7.21 (s, 2H), 6.96 (s, 1H), 5.40 (dd, J=5.2, 12.8 Hz, 1H), 4.40 (s, 2H), 3.96-3.88 (m, 2H), 3.78 (s, 2H), 2.97-2.86 (m, 1H), 2.81-2.70 (m, 1H), 2.64-2.58 (m, 1H), 2.13-1.99 (m, 1H), 1.23 (t, J=7.2 Hz, 3H).

### N-(6-(1,1-dioxido-4-oxo-1,2,5-thiadiazolidin-2-yl)-5-fluoro-7-hydroxynaphthalen-2-yl)-2-(4-((3-(2,6-dioxopiperidin-3-yl)-1-methyl-1H-indazol-6-yl)amino)phenyl)acetamide (Degrader 48)

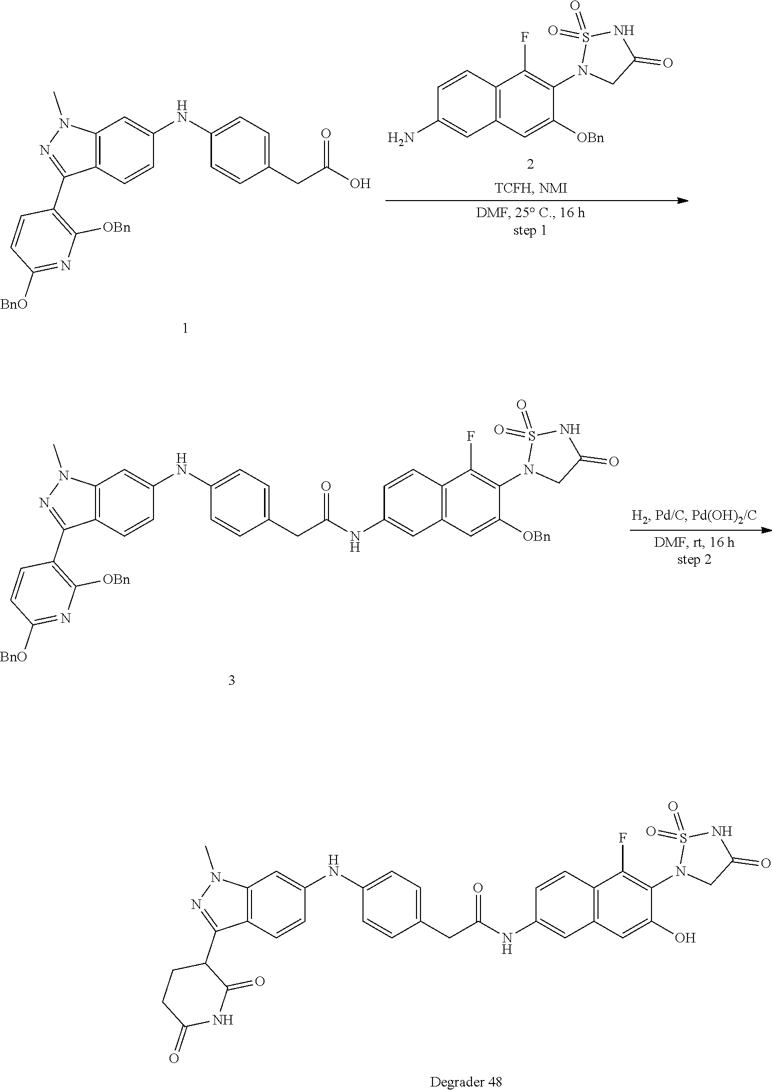 [SMILES: Cn1nc(-c2ccc(OCc3ccccc3)nc2OCc2ccccc2)c2ccc(Nc3ccc(CC(=O)O)cc3)cc21 ; Nc1ccc2c(F)c(N3CC(=O)NS3(=O)=O)c(OCc3ccccc3)cc2c1 ; Cn1nc(-c2ccc(OCc3ccccc3)nc2OCc2ccccc2)c2ccc(Nc3ccc(CC(=O)Nc4ccc5c(F)c(N6CC(=O)NS6(=O)=O)c(OCc6ccccc6)cc5c4)cc3)cc21 ; Cn1nc(C2CCC(=O)NC2=O)c2ccc(Nc3ccc(CC(=O)Nc4ccc5c(F)c(N6CC(=O)NS6(=O)=O)c(O)cc5c4)cc3)cc21]

### Step 1: N-(7-(benzyloxy)-6-(1,1-dioxido-4-oxo-1,2,5-thiadiazolidin-2-yl)-5-fluoronaphthalen-2-yl)-2-(4-((3-(2,6-bis(benzyloxy)pyridin-3-yl)-1-methyl-1H-indazol-6-yl)amino)phenyl)acetamide (3)

To a mixture of 2-(4-((3-(2,6-bis(benzyloxy)pyridin-3-yl)-1-methyl-1H-indazol-6-yl)amino)phenyl)acetic acid (1, 200 mg, 350.49 μmol, 1 eq), 5-(6-amino-3-(benzyloxy)-1-fluoronaphthalen-2-yl)-1,2,5-thiadiazolidin-3-one 1,1-dioxide (2, 180.65 mg, 350.49 μmol, TFA salt, 1 eq) and NMI (143.88 mg, 1.75 mmol, 139.69 μL, 5 eg) in DMF (5 mL) was added TCFH (245.85 mg, 876.22 μmol, 2.5 eq) at 0° C. The mixture was stirred at 20° C. for 16 h. The mixture was concentrated in vacuum. The residue was purified by reversed-phase flash (flow: 40 mL/min; gradient: from 40-100% MeCN in water (NH₃H₂O) over 10 min; column: Welch Ultimate XB_C18 20-40 μm; 120 Å) to afford N-(7-(benzyloxy)-6-(1,1-dioxido-4-oxo-1,2,5-thiadiazolidin-2-yl)-5-fluoronaphthalen-2-yl)-2-(4-((3-(2,6-bis(benzyloxy)pyridin-3-yl)-1-methyl-1H-indazol-6-yl)amino)phenyl)acetamide (3, 150 mg, 152.51 μmol, 44% yield) as yellow solid.

LCMS (ESI): m/z 954.3 [M+H]⁺

### Step 2: N-(6-(1,1-dioxido-4-oxo-1,2,5-thiadiazolidin-2-yl)-5-fluoro-7-hydroxynaphthalen-2-yl)-2-(4-((3-(2,6-dioxopiperidin-3-yl)-1-methyl-1H-indazol-6-yl)amino)phenyl)acetamide (Degrader 48)

To a solution of N-(7-(benzyloxy)-6-(1,1-dioxido-4-oxo-1,2,5-thiadiazolidin-2-yl)-5-fluoronaphthalen-2-yl)-2-(4-((3-(2,6-bis(benzyloxy)pyridin-3-yl)-1-methyl-1H-indazol-6-yl)amino)phenyl)acetamide (3, 100 mg, 104.82 μmol, 1 eq) in DMF (10 mL) was added Pd(OH)₂/C (10 mg, 10% purity) and Pd/C (10 mg, 10% purity) under H₂ (15 psi), the mixture was stirred at 20° C. for 16 h under H₂ (15 psi) atmosphere. The mixture was filtered and concentrated in vacuum. The residue was purified by prep-HPLC (flow: 25 mL/min; gradient: from 37-67% MeCN in water (0.1% TFA); column: Phenomenex Synergi C18 150×25 mm×10 um) to afford N-(6-(1,1-dioxido-4-oxo-1,2,5-thiadiazolidin-2-yl)-5-fluoro-7-hydroxynaphthalen-2-yl)-2-(4-((3-(2,6-dioxopiperidin-3-yl)-1-methyl-1H-indazol-6-yl)amino)phenyl)acetamide (Degrader 48, 59.89 mg, 74.89 μmol, 71% yield, TFA salt) as gray solid.

LCMS (ESI): m/z 686.1 [M+H]⁺

¹H NMR (400 MHz, DMSO-d₆) δ 10.85 (s, 1H), 10.53-10.27 (m, 2H), 8.48-8.25 (m, 1H), 8.19 (s, 1H), 7.87 (d, J=8.8 Hz, 1H), 7.52 (d, J=8.8 Hz, 1H), 7.47 (dd, J=2.0, 9.2 Hz, 1H), 7.27 (d, J=8.6 Hz, 2H), 7.16 (d, J=8.6 Hz, 2H), 7.06 (d, J=1.6 Hz, 1H), 6.96 (s, 1H), 6.84 (dd, J=1.8, 8.6 Hz, 1H), 4.41 (s, 2H), 4.26 (dd, J=5.2, 9.2 Hz, 1H), 3.84 (s, 3H), 3.63 (br s, 2H), 2.68-2.60 (m, 2H), 2.33-2.28 (m, 1H), 2.20-2.13 (m, 1H).

### N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-ethyl-2-oxo-2,3-dihydro-1H-benzo[d]imidazol-5-yl)phenyl)acetamide (Degrader 49)

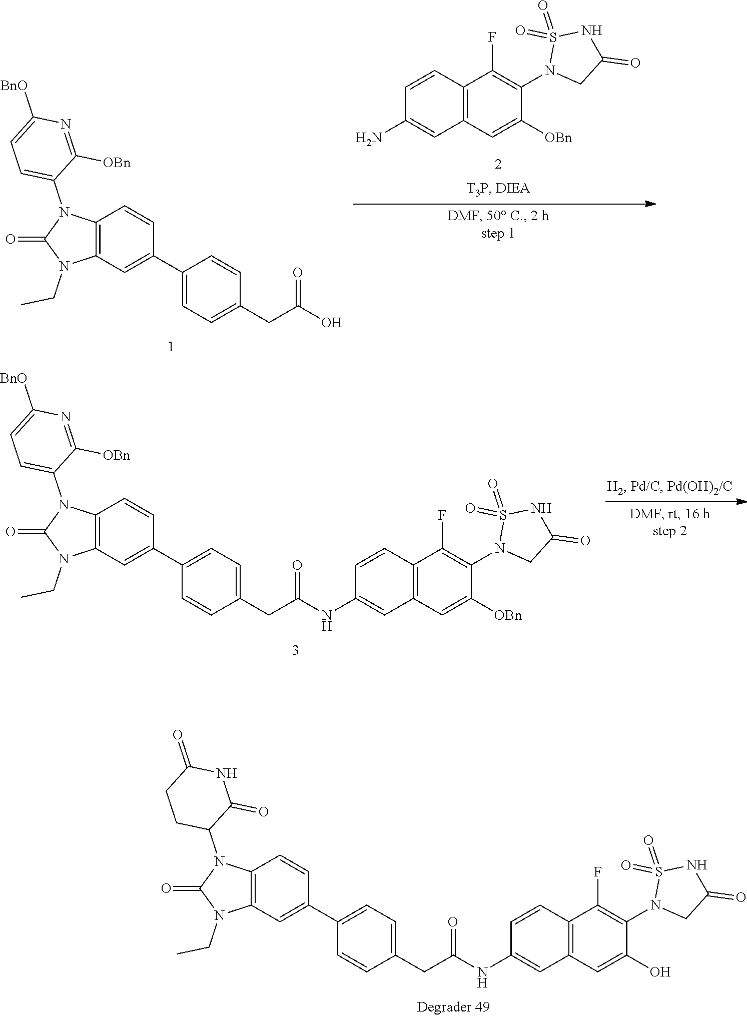 [SMILES: CCn1c(=O)n(-c2ccc(OCc3ccccc3)nc2OCc2ccccc2)c2ccc(-c3ccc(CC(=O)O)cc3)cc21 ; Nc1ccc2c(F)c(N3CC(=O)NS3(=O)=O)c(OCc3ccccc3)cc2c1 ; CCn1c(=O)n(-c2ccc(OCc3ccccc3)nc2OCc2ccccc2)c2ccc(-c3ccc(CC(=O)Nc4ccc5c(F)c(N6CC(=O)NS6(=O)=O)c(OCc6ccccc6)cc5c4)cc3)cc21 ; CCn1c(=O)n(C2CCC(=O)NC2=O)c2ccc(-c3ccc(CC(=O)Nc4ccc5c(F)c(N6CC(=O)NS6(=O)=O)c(O)cc5c4)cc3)cc21]

### Step 1: N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-ethyl-2-oxo-2,3-dihydro-1H-benzo[d]imidazol-5-yl)phenyl)acetamide (3)

To a solution of 2-(4-(1-(2,6-bis(benzyloxy)pyridin-3-yl)-3-ethyl-2-oxo-2,3-dihydro-1H-benzo[d]imidazol-5-yl)phenyl)acetic acid (1, 200 mg, 341.50 μmol, 1 eq) in DMF (1 mL) was added 5-(6-amino-3-(benzyloxy)-1-fluoronaphthalen-2-yl)-1,2,5-thiadiazolidin-3-one 1,1-dioxide (2, 211.23 mg, 409.80 μmol, 1.2 eq), DIEA (662.05 mg, 5.12 mmol, 892.25 μL, 15 eq) and T₃P (1.52 g, 2.39 mmol, 50% purity in EtOAc, 7 eq). The mixture was stirred at 50° C. for 2 h. The residue was purified by prep-HPLC (FlowRate: 25 mL/min; gradient: from 19-49% MeCN in water (10 mM NH₄HCO₃); column: Waters Xbridgem 150×25 mm×5 um) to afford N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-ethyl-2-oxo-2,3-dihydro-1H-benzo[d]imidazol-5-yl)phenyl)acetamide (3, 175 mg, 167.95 μmol, 49% yield) as white solid.

LCMS (ESI): m/z 969.7 [M+H]⁺

### Step 2: N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-ethyl-2-oxo-2,3-dihydro-1H-benzo[d]imidazol-5-yl)phenyl)acetamide (Degrader 49)

To a solution of N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-ethyl-2-oxo-2,3-dihydro-1H-benzo[d]imidazol-5-yl)phenyl)acetamide (3, 120 mg, 123.83 μmol, 1 eq) in DMF (10 mL) was added Pd/C (120 mg, 10% purity) and Pd(OH)₂/C (120 mg, 10% purity) at 25° C. under N₂, the mixture was stirred at 25° C. under H₂ (15 psi) for 16 h. The mixture was filtered and concentrated in vacuum. The residue was purified by prep-HPLC (FlowRate: 25 mL/min; gradient: from 32-62% MeCN in water (0.1% TFA); column: 3_Phenomenex Luna C18 75×30 mm×3 um) to afford N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-ethyl-2-oxo-2,3-dihydro-1H-benzo[d]imidazol-5-yl)phenyl)acetamide (Degrader 49 59.07 mg, 71.05 μmol, 57% yield, TFA salt).

LCMS (ESI): m/z 701.0 [M+H]⁺

¹H NMR (400 MHz, DMSO-d₆) δ 11.11 (s, 1H), 10.42 (s, 1H), 10.18-9.99 (m, 1H), 8.16 (s, 1H), 7.85 (d, J=9.2 Hz, 1H), 7.66 (d, J=8.0 Hz, 2H), 7.53 (d, J=1.2 Hz, 1H), 7.47-7.43 (m, 3H), 7.33 (dd, J=1.6, 8.4 Hz, 1H), 7.19 (d, J=8.4 Hz, 1H), 6.94 (s, 1H), 5.39 (dd, J=5.2, 12.8 Hz, 1H), 4.24 (s, 2H), 3.95 (q, J=6.8 Hz, 2H), 3.74 (s, 2H), 2.96-2.85 (m, 1H), 2.81-2.71 (m, 1H), 2.65-2.60 (m, 1H), 2.08-2.04 (m, 1H), 1.26 (t, J=7.2 Hz, 3H).

### 2-[3-[1-(2,6-dioxo-3-piperidyl)-3-methyl-2-oxo-benzimidazol-5-yl]-8-azabicyclo[3.2.1]octan-8-yl]-N-[5-fluoro-7-hydroxy-6-(1,1,4-trioxo-1,2,5-thiadiazolidin-2-yl)-2-naphthyl]acetamide (Degrader 50, Endo Isomer)

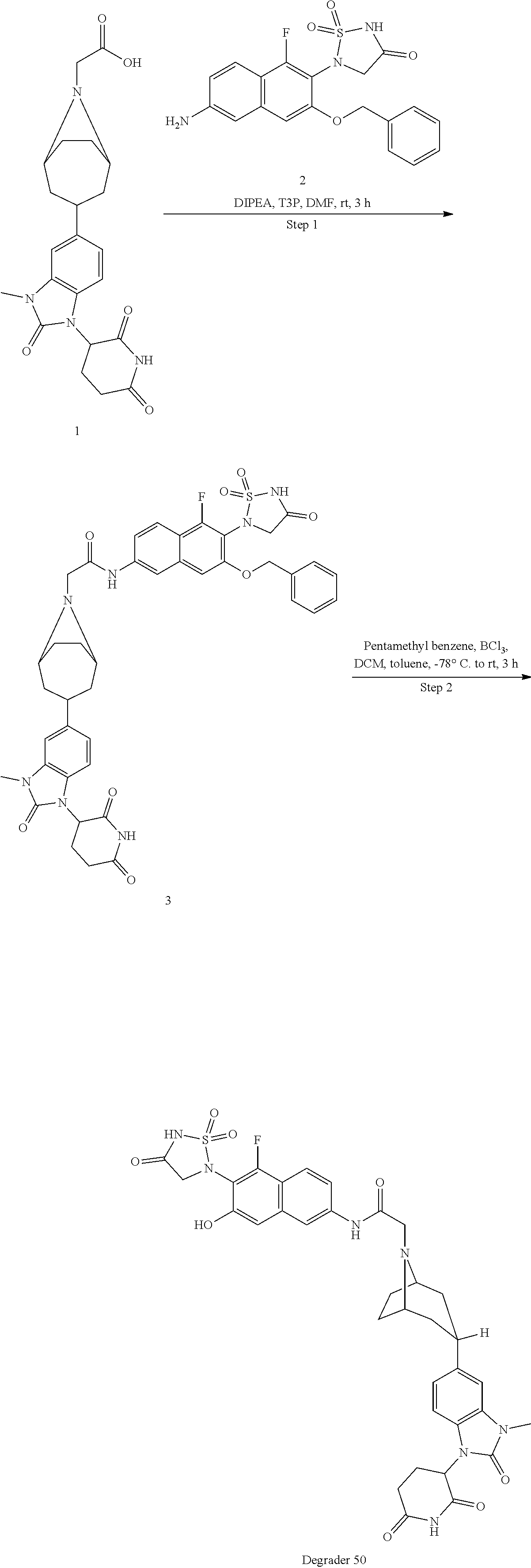 [SMILES: Cn1c(=O)n(C2CCC(=O)NC2=O)c2ccc(C3CC4CCC(C3)N4CC(=O)O)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(C3CC4CCC(C3)N4CC(=O)Nc3ccc4c(F)c(N5CC(=O)NS5(=O)=O)c(OCc5ccccc5)cc4c3)cc21 ; Cn1c(=O)n(C2CCC(=O)NC2=O)c2ccc(C3CC4CCC(C3)N4CC(=O)Nc3ccc4c(F)c(N5CC(=O)NS5(=O)=O)c(O)cc4c3)cc21]

Note: Configurations are arbitrarily assigned.

### Step 1: N-[7-benzyloxy-5-fluoro-6-(1,1,4-trioxo-1,2,5-thiadiazolidin-2-yl)-2-naphthyl]-2-[3-[1-(2,6-dioxo-3-piperidyl)-3-methyl-2-oxo-benzimidazol-5-yl]-8-azabicyclo[3.2.1]octan-8-yl]acetamide (3)

Into a 10 mL round-bottom flask containing a well-stirred solution of 2-[3-[1-(2,6-dioxo-3-piperidyl)-3-methyl-2-oxo-benzimidazol-5-yl]-8-azabicyclo[3.2.1]octan-8-yl]acetic acid (1, 200 mg, 277.42 μmol, TFA salt) in DMF (5 mL) was added DIPEA (107.56 mg, 832.25 μmol, 144.96 μL) and propane phosphonic acid anhydride 50 wt. % solution in EtOAc (176.54 mg, 277.42 μmol, 50% purity) and the reaction mixture was stirred at room temperature for 30 min. Then, 5-(6-amino-3-benzyloxy-1-fluoro-2-naphthyl)-1,1-dioxo-1,2,5-thiadiazolidin-3-one (2, 148.81 mg, 277.42 μmol, TFA salt) was added. The reaction mixture was stirred at ambient temperature. After 3 h, the volatiles were distilled off under reduced pressure to get N-[7-benzyloxy-5-fluoro-6-(1,1,4-trioxo-1,2,5-thiadiazolidin-2-yl)-2-naphthyl]-2-[3-[1-(2,6-dioxo-3-piperidyl)-3-methyl-2-oxo-benzimidazol-5-yl]-8-azabicyclo[3.2.1]octan-8-yl]acetamide (3, 250 mg, 111.38 μmol, 40% yield) as a brown gum.

LCMS (ES+): m/z 810.2 [M+H]⁺

### Step 2: 2-[3-[1-(2,6-dioxo-3-piperidyl)-3-methyl-2-oxo-benzimidazol-5-yl]-8-azabicyclo[3.2.1]octan-8-yl]-N-[5-fluoro-7-hydroxy-6-(1,1,4-trioxo-1,2,5-thiadiazolidin-2-yl)-2-naphthyl]acetamide (Degrader 50, Endo Isomer)

Into a 10 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[3-[1-(2,6-dioxo-3-piperidyl)-3-methyl-2-oxo-benzimidazol-5-yl]-8-azabicyclo[3.2.1]octan-8-yl]acetamide (3, 250 mg, 111.38 μmol), pentamethylbenzene (82.56 mg, 556.89 μmol) in anhydrous DCM (2 mL) and toluene (2 mL) was added a 1.0 M solution of BCl₃ in DCM (1.11 mmol, 1.11 mL) at −78° C. The reaction mixture was stirred at room temperature. After 3 h, the reaction mixture was quenched with 5% MeOH in DCM (5 mL) at −78° C. The volatiles were removed under reduced pressure to get the crude compound, which was purified by reverse phase column chromatography [Purification method: Column XSelect C18 (150×19), 5 micron; Mobile phase A: 0.1% TFA in water; Mobile phase B: MeCN] to obtain 2-[3-[1-(2,6-dioxo-3-piperidyl)-3-methyl-2-oxo-benzimidazol-5-yl]-8-azabicyclo[3.2.1]octan-8-yl]-N-[5-fluoro-7-hydroxy-6-(1,1,4-trioxo-1,2,5-thiadiazolidin-2-yl)-2-naphthyl]acetamide (Degrader 50, 40 mg, 43.18 μmol, 39% yield, TFA salt) as an off white solid.

LCMS (ES+): m/z 720.1 [M+H]⁺

¹HNMR (400 MHz, DMSO-d6): δ 11.12 (s, 1H), 10.71 (s, 1H), 10.04 (s, 1H), 9.65 (s, 1H), 8.18 (s, 1H), 7.92 (d, J=9.20 Hz, 1H), 7.44 (d, J=8.80 Hz, 1H), 7.39 (s, 1H), 7.22 (d, J=8.00 Hz, 1H), 7.09 (d, J=8.00 Hz, 1H), 7.02 (s, 1H), 5.40-5.35 (m, 1H), 4.18 (s, 2H), 4.14 (s, 2H), 4.03-3.95 (m, 2H), 3.39 (s, 3H), 3.29-3.27 (m, 1H), 2.91-2.87 (m, 1H), 2.74-2.66 (m, 6H), 2.08-1.96 (m, 3H), 1.76 (d, J=8.80 Hz, 2H).

### 2-[3-[1-(2,6-dioxo-3-piperidyl)-3-methyl-2-oxo-benzimidazol-5-yl]-8-azabicyclo[3.2.1]octan-8-yl]-N-[5-fluoro-7-hydroxy-6-(1,1,4-trioxo-1,2,5-thiadiazolidin-2-yl)-2-naphthyl]acetamide (Degrader 51, Exo Isomer)

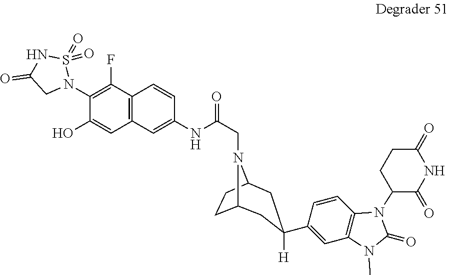 [SMILES: Cn1c(=O)n(C2CCC(=O)NC2=O)c2ccc(C3CC4CCC(C3)N4CC(=O)Nc3ccc4c(F)c(N5CC(=O)NS5(=O)=O)c(O)cc4c3)cc21]

Note: Exo isomer configuration is arbitrarily assigned.

2-[3-[1-(2,6-dioxo-3-piperidyl)-3-methyl-2-oxo-benzimidazol-5-yl]-8-azabicyclo[3.2.1]octan-8-yl]-N-[5-fluoro-7-hydroxy-6-(1,1,4-trioxo-1,2,5-thiadiazolidin-2-yl)-2-naphthyl]acetamide (Degrader 51, exo isomer) was synthesized following the same procedure as 2-[3-[1-(2,6-dioxo-3-piperidyl)-3-methyl-2-oxo-benzimidazol-5-yl]-8-azabicyclo[3.2.1]octan-8-yl]-N-[5-fluoro-7-hydroxy-6-(1,1,4-trioxo-1,2,5-thiadiazolidin-2-yl)-2-naphthyl]acetamide (Degrader 50). LCMS (ES+): m/z 720.0 [M+H]⁺

¹HNMR (400 MHz, DMSO-d6): δ 11.12 (s, 1H), 10.76 (s, 1H), 10.00 (s, 1H), 9.92 (s, 1H), 8.18 (s, 1H), 7.92 (d, J=8.80 Hz, 1H), 7.46 (dd, J=1.60, 8.80 Hz, 1H), 7.18 (s, 1H), 7.12-7.04 (m, 2H), 7.02 (s, 1H), 5.40-5.35 (m, 1H), 4.19 (s, 2H), 4.17 (s, 2H), 4.11 (d, J=5.20 Hz, 2H), 3.37 (s, 3H), 3.31-3.15 (m, 1H), 2.92-2.88 (m, 1H), 2.75-2.67 (m, 2H), 2.39-2.15 (m, 5H), 2.08-1.92 (m, 4H).

### 1-(6-(1,1-dioxido-4-oxo-1,2,5-thiadiazolidin-2-yl)-5-fluoro-7-hydroxynaphthalen-2-yl)-3-(2-(4-(1-(2,6-dioxopiperidin-3-yl)-3-methyl-2-oxo-2,3-dihydro-1H-benzo[d]imidazol-5-yl)piperidin-1-yl)ethyl)urea (Degrader 52)

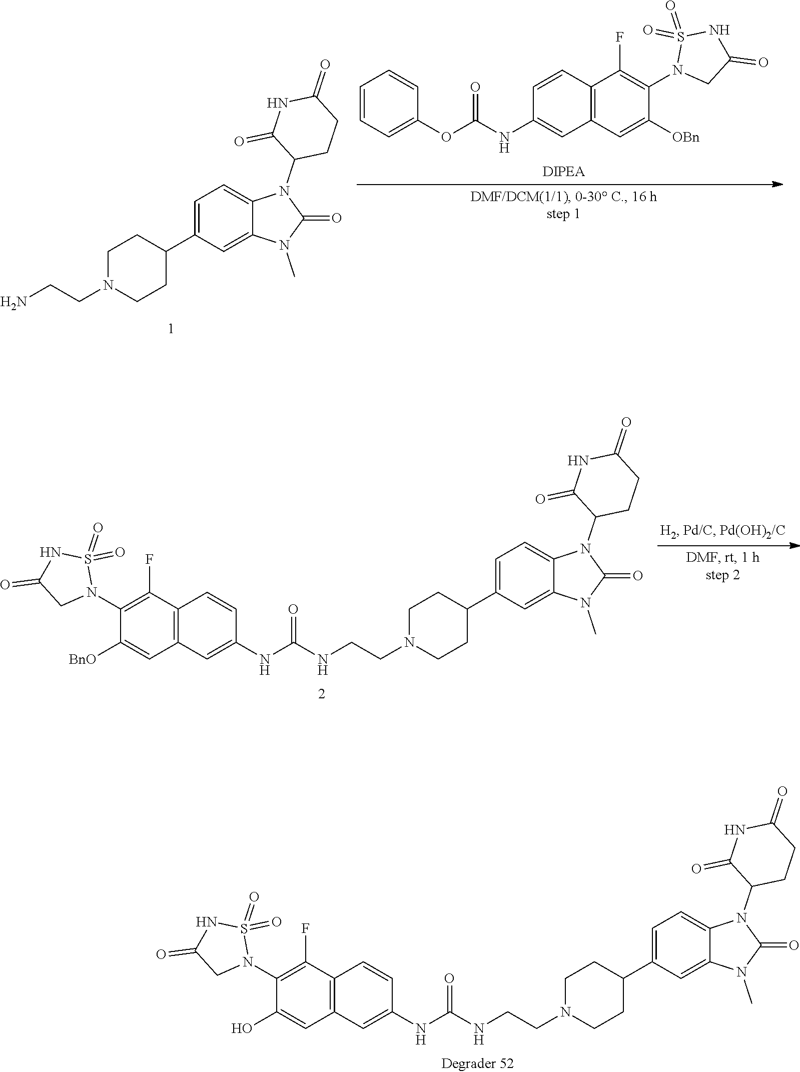

### Step 1: 1-(7-(benzyloxy)-6-(1,1-dioxido-4-oxo-1,2,5-thiadiazolidin-2-yl)-5-fluoronaphthalen-2-yl)-3-(2-(4-(1-(2,6-dioxopiperidin-3-yl)-3-methyl-2-oxo-2,3-dihydro-1H-benzo[d]imidazol-5-yl)piperidin-1-yl)ethyl)urea (3)

To a solution of 3-(5-(1-(2-aminoethyl)piperidin-4-yl)-3-methyl-2-oxo-2,3-dihydro-1H-benzo[d]imidazol-1-yl)piperidine-2,6-dione (1, 200 mg, 518.86 μmol, 1 eq) in DMF (3 mL) and DCM (3 mL) was added DIPEA (335.30 mg, 2.59 mmol, 451.88 μL, 5 eq). Then to the mixture was added phenyl (7-(benzyloxy)-6-(1,1-dioxido-4-oxo-1,2,5-thiadiazolidin-2-yl)-5-fluoronaphthalen-2-yl)carbamate (2, 270.59 mg, 518.86 μmol, 1 eq) at 0° C. The mixture was stirred at 30° C. for 16 h. The reaction mixture was filtered, then the filtrate was concentrated under reduced pressure and purified by prep-HPLC (flow: 25 mL/min; gradient: from 21-51% MeCN-water (0.1% TFA) over 10 min; column: Phenomenex Synergi C18 150×25 mm×10 um) to afford 1-(7-(benzyloxy)-6-(1,1-dioxido-4-oxo-1,2,5-thiadiazolidin-2-yl)-5-fluoronaphthalen-2-yl)-3-(2-(4-(1-(2,6-dioxopiperidin-3-yl)-3-methyl-2-oxo-2,3-dihydro-1H-benzo[d]imidazol-5-yl)piperidin-1-yl)ethyl)urea (3, 125 mg, 150.70 μmol, 29% yield) as a white solid.

LCMS (ESI): m/z 813.4 [M+H]⁺

¹H NMR (400 MHz, d₆-DMSO) δ 11.10 (s, 1H), 9.49-8.95 (m, 2H), 8.04 (s, 1H), 7.90-7.79 (m, 1H), 7.55 (d, J=6.8 Hz, 2H), 7.49-7.22 (m, 5H), 7.16 (s, 1H), 7.11-7.00 (m, 2H), 6.93 (d, J=7.6 Hz, 1H), 6.76-6.61 (m, 1H), 5.36 (dd, J=4.8, 12.0 Hz, 1H), 5.26 (s, 2H), 4.23-4.14 (m, 2H), 3.73 (d, J=10.4 Hz, 2H), 3.34 (s, 3H), 3.28 (s, 2H), 3.19-3.09 (m, 2H), 2.98-2.85 (m, 2H), 2.74-2.63 (m, 2H), 2.11-1.84 (m, 5H).

### Step 2: 1-(6-(1,1-dioxido-4-oxo-1,2,5-thiadiazolidin-2-yl)-5-fluoro-7-hydroxynaphthalen-2-yl)-3-(2-(4-(1-(2,6-dioxopiperidin-3-yl)-3-methyl-2-oxo-2,3-dihydro-1H-benzo[d]imidazol-5-yl)piperidin-1-yl)ethyl)urea (Degrader 52)

To a solution of 1-(7-(benzyloxy)-6-(1,1-dioxido-4-oxo-1,2,5-thiadiazolidin-2-yl)-5-fluoronaphthalen-2-yl)-3-(2-(4-(1-(2,6-dioxopiperidin-3-yl)-3-methyl-2-oxo-2,3-dihydro-1H-benzo[d]imidazol-5-yl)piperidin-1-yl)ethyl)urea (3, 100 mg, 123.02 μmol, 1 eq) in DMF (5 mL) were added Pd/C (100.00 mg, 10% purity) and Pd(OH)₂/C (100 mg, 10% purity) under N₂ atmosphere. The suspension was degassed and purged with H₂ 3 times. The mixture was stirred at 20° C. for 1 hr under H₂ (15 psi). The reaction mixture was filtered, then the filtrate was concentrated under reduced pressure and purified by prep-HPLC (flow: 25 mL/min; gradient: from 12-42% MeCN-water (0.1% TFA) over 7 min; column: 3_Phenomenex Luna C18 75×30 mm×3 um) to afford 1-(6-(1,1-dioxido-4-oxo-1,2,5-thiadiazolidin-2-yl)-5-fluoro-7-hydroxynaphthalen-2-yl)-3-(2-(4-(1-(2,6-dioxopiperidin-3-yl)-3-methyl-2-oxo-2,3-dihydro-1H-benzo[d]imidazol-5-yl)piperidin-1-yl)ethyl)urea (Degrader 52, 69.26 mg, 81.94 μmol, 67% yield, TFA salt) as an off-white solid.

LCMS (ESI): m/z 723.2 [M+H]⁺

¹H NMR (400 MHz, d₆-DMSO) δ 11.10 (s, 1H), 9.78 (s, 1H), 9.13-9.07 (m, 1H), 7.91 (s, 1H), 7.79 (d, J=8.8 Hz, 1H), 7.35 (d, J=7.6 Hz, 1H), 7.11-7.02 (m, 2H), 6.97-6.85 (m, 2H), 6.57 (d, J=3.2 Hz, 1H), 5.36 (dd, J=4.8, 12.4 Hz, 1H), 4.15 (s, 2H), 3.73 (d, J=10.8 Hz, 2H), 3.35 (s, 3H), 3.28 (s, 2H), 3.18-3.09 (m, 2H), 2.90 (d, J=10.8 Hz, 2H), 2.72 (d, J=3.6 Hz, 1H), 2.65 (s, 2H), 2.14-1.82 (m, 6H).

### 1-(6-(1,1-dioxido-4-oxo-1,2,5-thiadiazolidin-2-yl)-5-fluoro-7-hydroxynaphthalen-2-yl)-3-(2-(1-(1-(2,6-dioxopiperidin-3-yl)-2-oxo-1,2-dihydrobenzo[cd]indol-6-yl)piperidin-4-yl)ethyl)urea (Degrader 53)

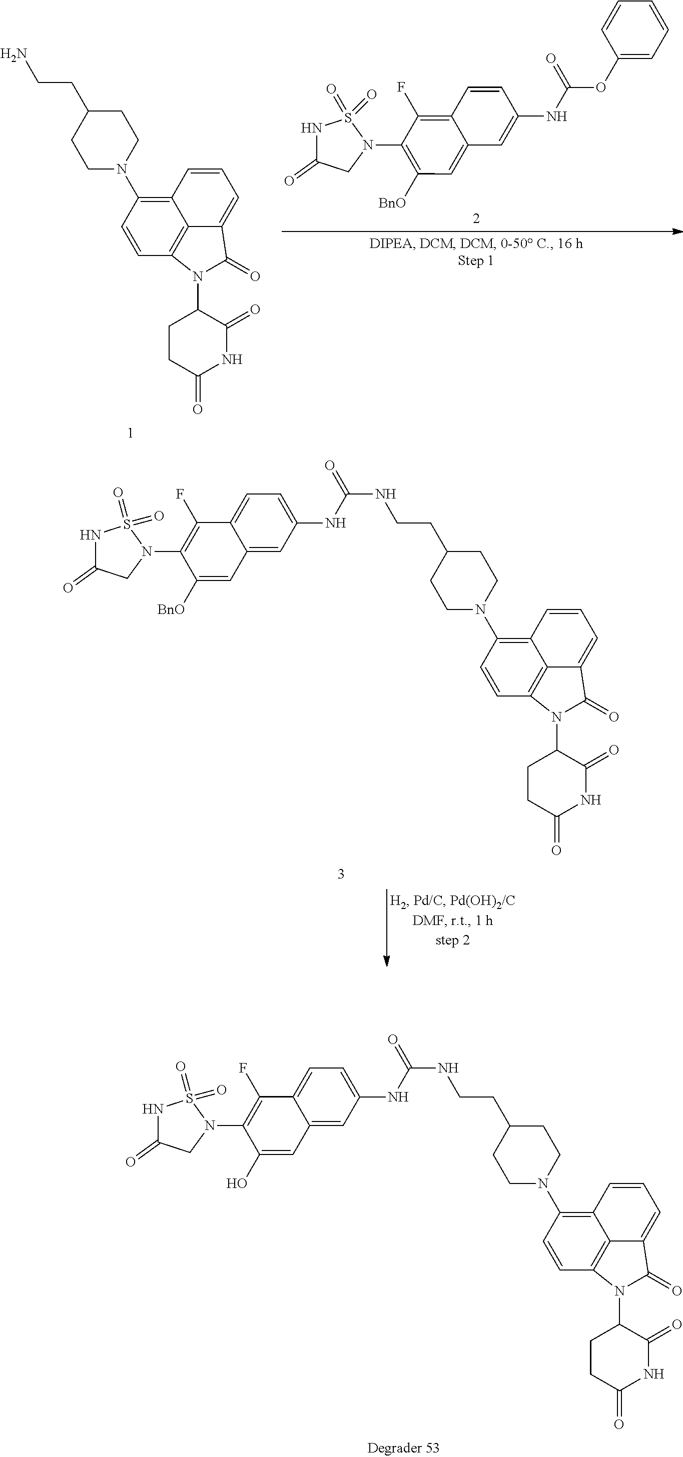 [SMILES: NCCC1CCN(c2ccc3c4c(cccc24)C(=O)N3C2CCC(=O)NC2=O)CC1 ; O=C1CN(c2c(OCc3ccccc3)cc3cc(NC(=O)Oc4ccccc4)ccc3c2F)S(=O)(=O)N1 ; O=C1CCC(N2C(=O)c3cccc4c(N5CCC(CCNC(=O)Nc6ccc7c(F)c(N8CC(=O)NS8(=O)=O)c(OCc8ccccc8)cc7c6)CC5)ccc2c34)C(=O)N1 ; O=C1CCC(N2C(=O)c3cccc4c(N5CCC(CCNC(=O)Nc6ccc7c(F)c(N8CC(=O)NS8(=O)=O)c(O)cc7c6)CC5)ccc2c34)C(=O)N1]

### Step 1: 1-(7-(benzyloxy)-6-(1,1-dioxido-4-oxo-1,2,5-thiadiazolidin-2-yl)-5-fluoronaphthalen-2-yl)-3-(2-(1-(1-(2,6-dioxopiperidin-3-yl)-2-oxo-1,2-dihydrobenzo[cd]indol-6-yl)piperidin-4-yl)ethyl)urea (3)

To a solution of 3-(6-(4-(2-aminoethyl)piperidin-1-yl)-2-oxobenzo[cd]indol-1 (2H)-yl)piperidine-2,6-dione (1, 100 mg, 246.02 μmol, 1 eq) and DIPEA (158.98 mg, 1.23 mmol, 214.25 μL, 5 eq) in DMF (2 mL) and DCM (2 mL) was added phenyl (7-(benzyloxy)-6-(1,1-dioxido-4-oxo-1,2,5-thiadiazolidin-2-yl)-5-fluoronaphthalen-2-yl)carbamate (2, 128.30 mg, 246.02 μmol, 1 eq) at 0° C. The mixture was stirred at 50° C. for 16 h. The reaction mixture was filtered, then the filtrate was concentrated under reduced pressure and purified by prep-HPLC (flow: 25 mL/min; gradient: from 30-60% MeCN-water (0.1% TFA) over 8 min; column: 3_Phenomenex Luna C18 75×30 mm×3 um) to afford 1-(7-(benzyloxy)-6-(1,1-dioxido-4-oxo-1,2,5-thiadiazolidin-2-yl)-5-fluoronaphthalen-2-yl)-3-(2-(1-(1-(2,6-dioxopiperidin-3-yl)-2-oxo-1,2-dihydrobenzo[cd]indol-6-yl)piperidin-4-yl)ethyl)urea (3, 100 mg, 117.52 μmol, 48% yield) as a yellow solid.

LCMS (ESI): m/z 834.2 [M+H]⁺

¹H NMR (400 MHz, d₆-DMSO) δ 11.11 (s, 1H), 8.82 (s, 1H), 8.25 (d, J=8.4 Hz, 1H), 8.14-8.00 (m, 2H), 7.87-7.77 (m, 2H), 7.53 (d, J=7.2 Hz, 2H), 7.41-7.30 (m, 4H), 7.21 (s, 1H), 7.05-6.93 (m, 2H), 6.34 (s, 1H), 5.41 (dd, J=5.2, 12.4 Hz, 1H), 5.26 (s, 2H), 4.38 (s, 2H), 3.24 (d, J=4.8 Hz, 3H), 2.97-2.89 (m, 1H), 2.82-2.70 (m, 3H), 2.67 (d, J=1.6 Hz, 1H), 2.62 (s, 1H), 2.12-2.03 (m, 1H), 1.90 (d, J=4.4 Hz, 2H), 1.55 (s, 5H).

### Step 2: 1-(6-(1,1-dioxido-4-oxo-1,2,5-thiadiazolidin-2-yl)-5-fluoro-7-hydroxynaphthalen-2-yl)-3-(2-(1-(1-(2,6-dioxopiperidin-3-yl)-2-oxo-1,2-dihydrobenzo[cd]indol-6-yl)piperidin-4-yl)ethyl)urea (Degrader 53)

To a solution of 1-(7-(benzyloxy)-6-(1,1-dioxido-4-oxo-1,2,5-thiadiazolidin-2-yl)-5-fluoronaphthalen-2-yl)-3-(2-(1-(1-(2,6-dioxopiperidin-3-yl)-2-oxo-1,2-dihydrobenzo[cd]indol-6-yl)piperidin-4-yl)ethyl)urea (3, 90 mg, 107.93 μmol, 1 eq) in DMF (5 mL) were added Pd(OH)₂/C (90 mg, 10% purity) and Pd/C (90 mg, 10% purity) under N₂ atmosphere. The suspension was degassed and purged with H₂ 3 times. The mixture was stirred at 20° C. for 1 hr under H₂ (15 psi). The reaction mixture was filtered, then the filtrate was concentrated under reduced pressure and purified by purified by prep-HPLC (flow: 25 mL/min; gradient: from 20-50% MeCN-water (0.1% TFA) over 10 min; column: Phenomenex Synergi C18 150×25 mm×10 um) to afford 1-(6-(1,1-dioxido-4-oxo-1,2,5-thiadiazolidin-2-yl)-5-fluoro-7-hydroxynaphthalen-2-yl)-3-(2-(1-(1-(2,6-dioxopiperidin-3-yl)-2-oxo-1,2-dihydrobenzo[cd]indol-6-yl)piperidin-4-yl)ethyl)urea (Degrader 53, 45.36 mg, 52.35 μmol, 49% yield, TFA salt) as a yellow solid.

LCMS (ESI): m/z 744.4 [M+H]⁺

¹H NMR (400 MHz, d₆-DMSO) δ 11.11 (s, 1H), 10.51-10.06 (m, 1H), 8.75 (s, 1H), 8.33-8.20 (m, 1H), 8.09 (d, J=4.4 Hz, 1H), 7.93 (s, 1H), 7.89-7.75 (m, 2H), 7.36-7.21 (m, 1H), 7.08-6.87 (m, 3H), 6.47-6.21 (m, 1H), 5.51-5.32 (m, 1H), 4.40 (s, 2H), 3.50-3.37 (m, 3H), 3.24 (s, 3H), 2.97-2.90 (m, 1H), 2.77 (td, J=1.6, 3.6 Hz, 2H), 2.14-2.05 (m, 1H), 1.91 (d, J=5.2 Hz, 2H), 1.55 (s, 5H).

### N1-(6-(1,1-dioxido-4-oxo-1,2,5-thiadiazolidin-2-yl)-5-fluoro-7-hydroxynaphthalen-2-yl)-N4-((1-(2,6-dioxopiperidin-3-yl)-2-oxo-1,2-dihydrobenzo[cd]indol-6-yl)methyl)succinamide (Degrader 54)

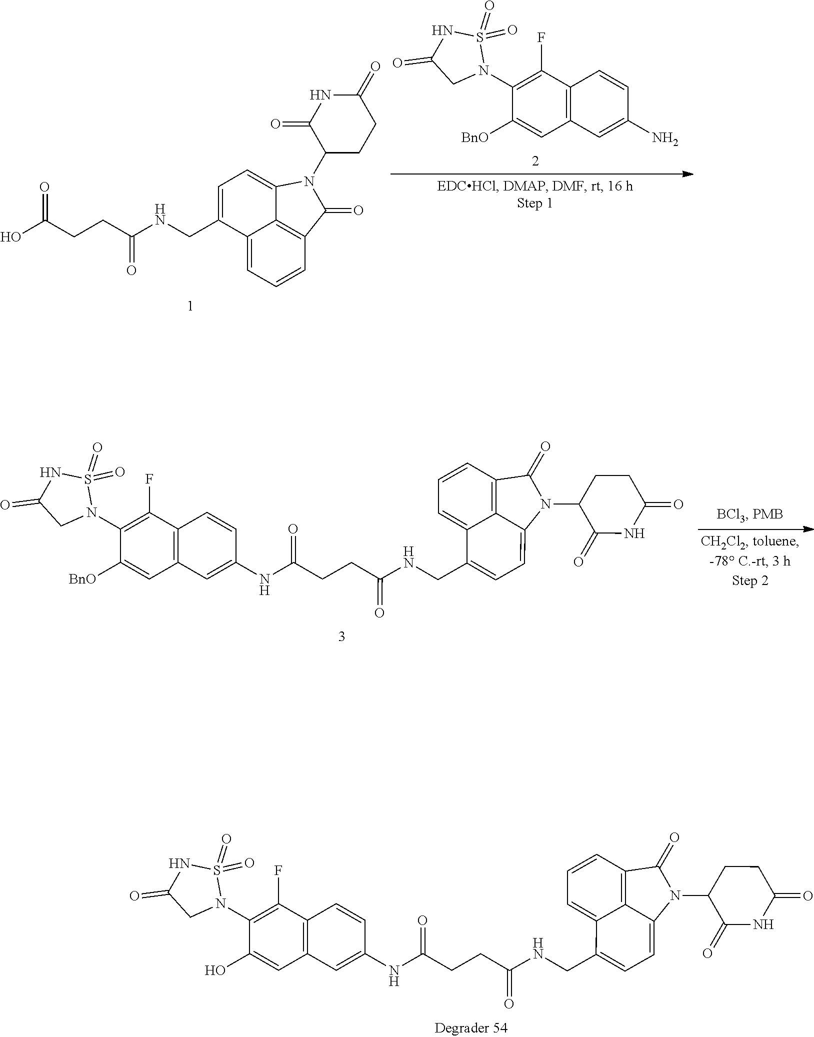

### Step 1: N1-(7-(benzyloxy)-6-(1,1-dioxido-4-oxo-1,2,5-thiadiazolidin-2-yl)-5-fluoronaphthalen-2-yl)-N4-((1-(2,6-dioxopiperidin-3-yl)-2-oxo-1,2-dihydrobenzo[cd]indol-6-yl)methyl)succinamide (3)

Into a 50 mL single-neck round-bottom flask containing a well-stirred solution of 4-[[1-(2,6-dioxo-3-piperidyl)-2-oxo-benzo[cd]indol-6-yl]methylamino]-4-oxo-butanoic acid (1, 200 mg, 433.33 μmol) in anhydrous DMF (3 mL) were added EDC·HCl (249.21 mg, 1.30 mmol), DMAP (264.69 mg, 2.17 mmol) and the resulting mixture was stirred at room temperature for 1 h. Subsequently 5-(6-amino-3-benzyloxy-1-fluoro-2-naphthyl)-1,1-dioxo-1,2,5-thiadiazolidin-3-one (2, 315.32 mg, 519.99 μmol, TFA salt) was added and stirring was continued at ambient temperature for 16 h. The reaction mixture was concentrated under reduced pressure and acidified with 10 mL of 1.5 N HCl solution. The solid and filtered, washed with water (5.0 mL) and dried under vacuum to afford N′-[7-benzyloxy-5-fluoro-6-(1,1,4-trioxo-1,2,5-thiadiazolidin-2-yl)-2-naphthyl]-N-[[1-(2,6-dioxo-3-piperidyl)-2-oxo-benzo[cd]indol-6-yl]methyl]butanediamide (3, 300 mg, 108.60 μmol, 25% yield) as yellow solid.

LCMS (ES+): m/z 793.0 [M+H]⁺

### Step 2: N1-(6-(1,1-dioxido-4-oxo-1,2,5-thiadiazolidin-2-yl)-5-fluoro-7-hydroxynaphthalen-2-yl)-N4-((1-(2,6-dioxopiperidin-3-yl)-2-oxo-1,2-dihydrobenzo[cd]indol-6-yl)methyl)succinamide (Degrader 54)

Into a 50 mL single-neck round-bottom flask containing a well-stirred solution of N′-[7-benzyloxy-5-fluoro-6-(1,1,4-trioxo-1,2,5-thiadiazolidin-2-yl)-2-naphthyl]-N-[[1-(2,6-dioxo-3-piperidyl)-2-oxo-benzo[cd]indol-6-yl]methyl]butanediamide (3, 300 mg, 108.60 μmol) and pentamethylbenzene (80.50 mg, 543.02 μmol) in anhydrous DCM (8 mL) and toluene (8 mL) was added BCl₃ (1.0 M solution in DCM, 2.17 mmol, 2.17 mL) at −78° C. The resultant reaction mixture was stirred at ambient temperature for 3 h. The reaction mixture was quenched with 1 mL of 5% MeOH in DCM at −78° C. The reaction mixture was concentrated under reduced pressure and washed with MTBE (10 mL) to obtain the crude material that was purified by reverse-phase preparative HPLC [Column: X Select C18 (250×19) mm, 5 μm; Mobile Phase A: 0.1% Formic acid in water and Mobile Phase B: Acetonitrile] to afford N1-(6-(1,1-dioxido-4-oxo-1,2,5-thiadiazolidin-2-yl)-5-fluoro-7-hydroxynaphthalen-2-yl)-N4-((1-(2,6-dioxopiperidin-3-yl)-2-oxo-1,2-dihydrobenzo[cd]indol-6-yl)methyl)succinamide (Degrader 54, 18 mg, 25.24 μmol, 23% yield) as off white solid.

LCMS (ES−): m/z 701.0 [M−H]⁻

¹H-NMR (400 MHz, DMSO-d6): δ 11.15 (s, 1H), 10.17 (s, 1H), 9.90 (brs, 1H), 8.38 (d, J=8.00 Hz, 1H), 8.11 (d, J=7.20 Hz, 2H), 7.87-7.78 (m, 2H), 7.44 (d, J=7.60 Hz, 1H), 7.38 (s, 1H), 7.10 (d, J=7.20 Hz, 1H), 6.92 (s, 1H), 5.45 (dd, J=5.60, 12.80 Hz, 1H), 4.72 (s, 2H), 4.08 (s, 2H), 3.01-2.90 (m, 1H), 2.78-2.62 (m, 4H), 2.11-2.08 (m, 1H).

N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-6-fluoro-3-methyl-2-oxo-2,3-dihydro-1H-benzo[d]imidazol-5-yl)piperazin-1-yl)acetamide (Degrader 55)

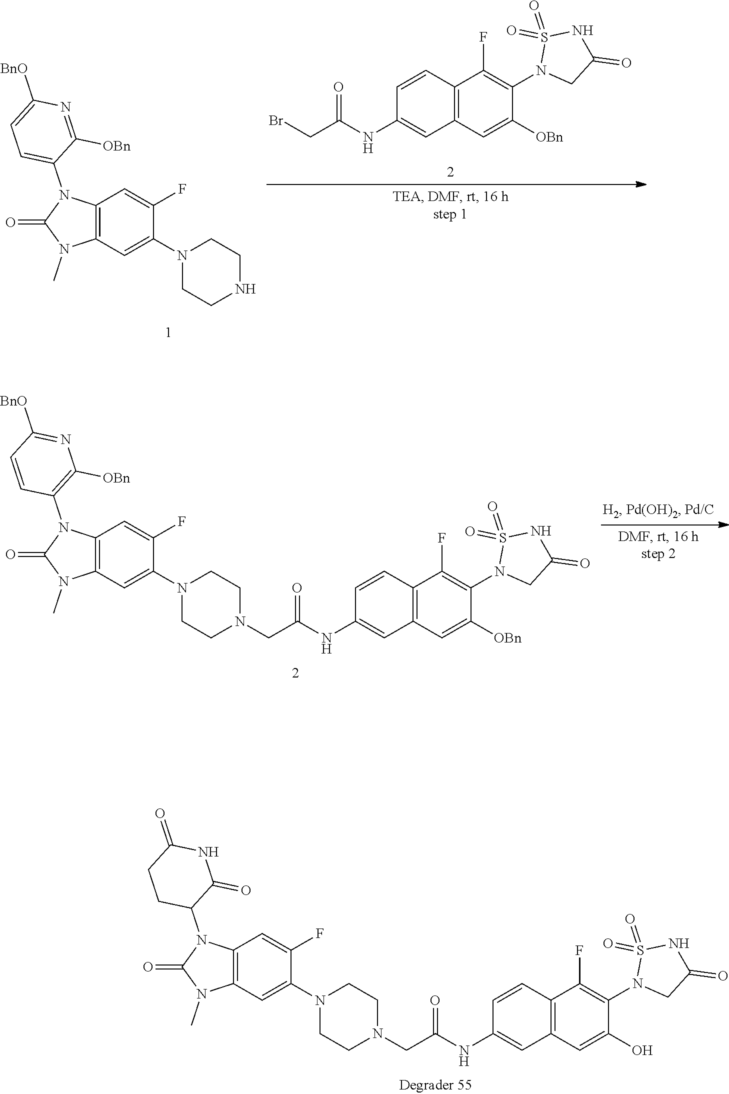 [SMILES: Cn1c(=O)n(-c2ccc(OCc3ccccc3)nc2OCc2ccccc2)c2cc(F)c(N3CCNCC3)cc21 ; O=C(CBr)Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2cc(F)c(N3CCN(CC(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2cc(F)c(N3CCN(CC(=O)Nc4ccc5c(F)c(N6CC(=O)NS6(=O)=O)c(O)cc5c4)CC3)cc21]

### Step 1: N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-6-fluoro-3-methyl-2-oxo-2,3-dihydro-1H-benzo[d]imidazol-5-yl)piperazin-1-yl)acetamide (3)

To a solution of 1-(2,6-bis(benzyloxy)pyridin-3-yl)-6-fluoro-3-methyl-5-(piperazin-1-yl)-1H-benzo[d]imidazol-2 (3H)-one (1, 0.3 g, 555.97 μmol) in DMF (5 mL) were added TEA (168.78 mg, 1.67 mmol, 232.47 μL, 3 eq) and a solution of N-(7-(benzyloxy)-6-(1,1-dioxido-4-oxo-1,2,5-thiadiazolidin-2-yl)-5-fluoronaphthalen-2-yl)-2-bromoacetamide (2, 290.41 mg, 555.97 μmol, 1 eq) in DMF (5 mL) at 0-10° C. The mixture was stirred at 30° C. for 16 h. The mixture was combined with another 45 mg batch and filtered. The filtrate was purified by prep-HPLC (flow: 25 mL/min; gradient: from 28-58% MeCN in water (0.05% ammonia hydroxide v/v) over 10 min; column: Waters Xbridge 150×25 mm×5 um) to afford N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-6-fluoro-3-methyl-2-oxo-2,3-dihydro-1H-benzo[d]imidazol-5-yl)piperazin-1-yl)acetamide (3, 100 mg, 100.91 μmol, 18% yield) as a white solid.

LCMS (ESI): m/z 981.6 [M+H]⁺

### Step 2: N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-6-fluoro-3-methyl-2-oxo-2,3-dihydro-1H-benzo[d]imidazol-5-yl)piperazin-1-yl)acetamide (Degrader 55)

To a solution of N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-6-fluoro-3-methyl-2-oxo-2,3-dihydro-1H-benzo[d]imidazol-5-yl)piperazin-1-yl)acetamide (3, 100 mg, 101.93 μmol) in DMF (3 mL) were added Pd/C (40 mg, 10% purity) and Pd(OH)₂ (40 mg, 10% purity). The mixture was stirred at 30° C. for 16 hr under H₂ (15 psi). The mixture was filtered and washed with DMF (2 mL×3), the filtrate was concentrated to give a residue. The residue was purified by prep-HPLC (flow: 25 mL/min; gradient: from 12-42% MeCN in water (0.1% TFA) over 10 min; column: 3_Phenomenex luna C18 150×25 mm×1 um) to afford N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-6-fluoro-3-methyl-2-oxo-2,3-dihydro-1H-benzo[d]imidazol-5-yl)piperazin-1-yl)acetamide (Degrader 55, 18 mg, 21.56 μmol, 21% yield, TFA salt) as a white solid.

LCMS (ESI): m/z 713.0 [M+H]+

¹H NMR (400 MHz, DMSO-d6) δ 11.08 (s, 1H), 10.82 (s, 1H), 10.23 (s, 1H), 9.85 (s, 1H), 8.12 (s, 1H), 7.91 (d, J=8.8 Hz, 1H), 7.49 (d, J=9.6 Hz, 1H), 7.25-7.18 (m, 1H), 7.09-6.99 (m, 2H), 5.36-5.31 (m, 1H), 4.33 (s, 2H), 4.12 (s, 3H), 3.72-3.68 (m, 4H), 3.37 (s, 3H), 3.29-3.23 (m, 4H), 2.89-2.84 (m, 1H), 2.76-2.72 (m, 1H), 2.64-2.60 (m, 1H), 2.04-1.95 (m, 1H).

### N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-4-fluoro-3-methyl-2-oxo-2,3-dihydro-1H-benzo[d]imidazol-5-yl)-3-fluorophenyl)acetamide (Degrader 56)

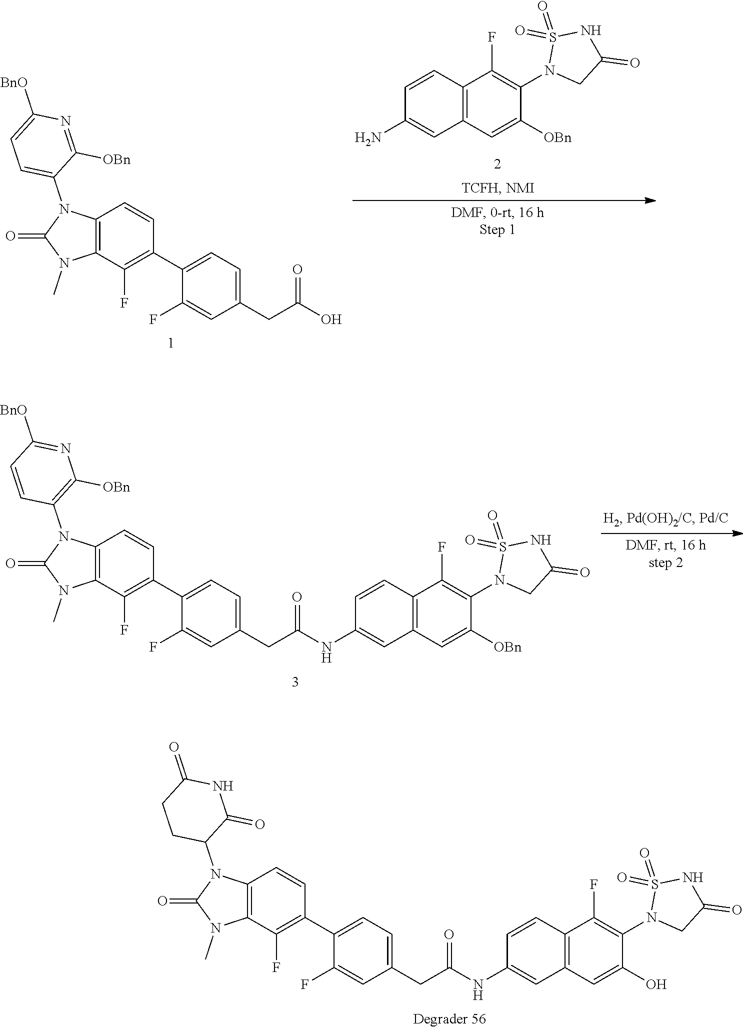 [SMILES: Cn1c(=O)n(-c2ccc(OCc3ccccc3)nc2OCc2ccccc2)c2ccc(-c3ccc(CC(=O)O)cc3F)c(F)c21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(-c3ccc(CC(=O)Nc4ccc5c(F)c(N6CC(=O)NS6(=O)=O)c(OCc6ccccc6)cc5c4)cc3F)c(F)c21 ; Cn1c(=O)n(C2CCC(=O)NC2=O)c2ccc(-c3ccc(CC(=O)Nc4ccc5c(F)c(N6CC(=O)NS6(=O)=O)c(O)cc5c4)cc3F)c(F)c21]

### Step 1: N-(7-benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-4-fluoro-3-methyl-2-oxo-2,3-dihydro-1H-benzo[d]imidazol-5-yl)-3-fluorophenyl)acetamide (3)

To a solution of 2-(4-(1-(2,6-bis(benzyloxy)pyridin-3-yl)-4-fluoro-3-methyl-2-oxo-2,3-dihydro-1H-benzo[d]imidazol-5-yl)-3-fluorophenyl)acetic acid (1, 160 mg, 263.33 μmol, 1 eq), 5-(6-amino-3-(benzyloxy)-1-fluoronaphthalen-2-yl)-1,2,5-thiadiazolidin-3-one 1,1-dioxide (2, 135.73 mg, 263.33 μmol, 1 eq, TFA salt) and NMJ (108.10 mg, 1.32 mmol, 104.95 μL, 5 eq) in DMF (2 mL) was added TCFH (184.71 mg, 658.33 μmol, 2.5 eq) at 0° C. The mixture was stirred at 20° C. for 2 h. The mixture was filtered and concentrated in vacuum. The residue was purified by prep-HPLC (flow: 25 mL/min; gradient: from 30-60% MeCN in water (0.05% ammonia hydroxide) over 12 min; column: Waters Xbridge 150×25 mm×5 um) to afford N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-4-fluoro-3-methyl-2-oxo-2,3-dihydro-1H-benzo[d]imidazol-5-yl)-3-fluorophenyl)acetamide (3, 140 mg, 139.86 μmol, 53% yield) as a white solid.

LCMS (ESI): m/z 991.4 [M+H]⁺

### Step 2: N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-4-fluoro-3-methyl-2-oxo-2,3-dihydro-1H-benzo[d]imidazol-5-yl)-3-fluorophenyl)acetamide (Degrader 56)

To a solution of N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-4-fluoro-3-methyl-2-oxo-2,3-dihydro-1H-benzo[d]imidazol-5-yl)-3-fluorophenyl)acetamide (3, 90 mg, 90.82 μmol, 1 eq) in DMF (9 mL) were added Pd/C (90 mg, 10% purity) and Pd(OH)₂/C (90 mg, 10% purity) under N₂ atmosphere. The suspension was degassed and purged with H₂ 3 times. The mixture was stirred under H₂ (15 psi) at 20° C. for 16 h. The mixture was filtered and concentrated in vacuum. The residue was purified by prep-HPLC (flow: 25 mL/min; gradient: from 32-62% MeCN in water (0.1% TFA) over 10 min; column: 3_Phenomenex Luna C18 75×30 mm×3 um) to afford N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-4-fluoro-3-methyl-2-oxo-2,3-dihydro-1H-benzo[d]imidazol-5-yl)-3-fluorophenyl)acetamide (Degrader 56, 82.8 mg, 97.97 μmol, TFA salt) as a pink solid.

LCMS (ESI): m/z 723.4 [M+H]⁺

¹H NMR (400 MHz, d₆-DMSO) δ 11.16 (s, 1H), 10.70-10.44 (m, 2H), 8.20 (s, 1H), 7.89 (d, J=9.2 Hz, 1H), 7.51-7.40 (m, 2H), 7.37-7.29 (m, 2H), 7.14-7.05 (m, 2H), 6.98 (s, 1H), 5.45 (dd, J=5.2, 12.8 Hz, 1H), 4.48 (s, 2H), 3.82 (s, 2H), 3.52 (d, J=1.6 Hz, 3H), 2.97-2.87 (m, 1H), 2.80-2.72 (m, 1H), 2.71-2.65 (m, 1H), 2.13-2.02 (m, 1H).

N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-4-fluoro-3-methyl-2-oxo-2,3-dihydro-1H-benzo[d]imidazol-5-yl)piperazin-1-yl)acetamide (Degrader 57)

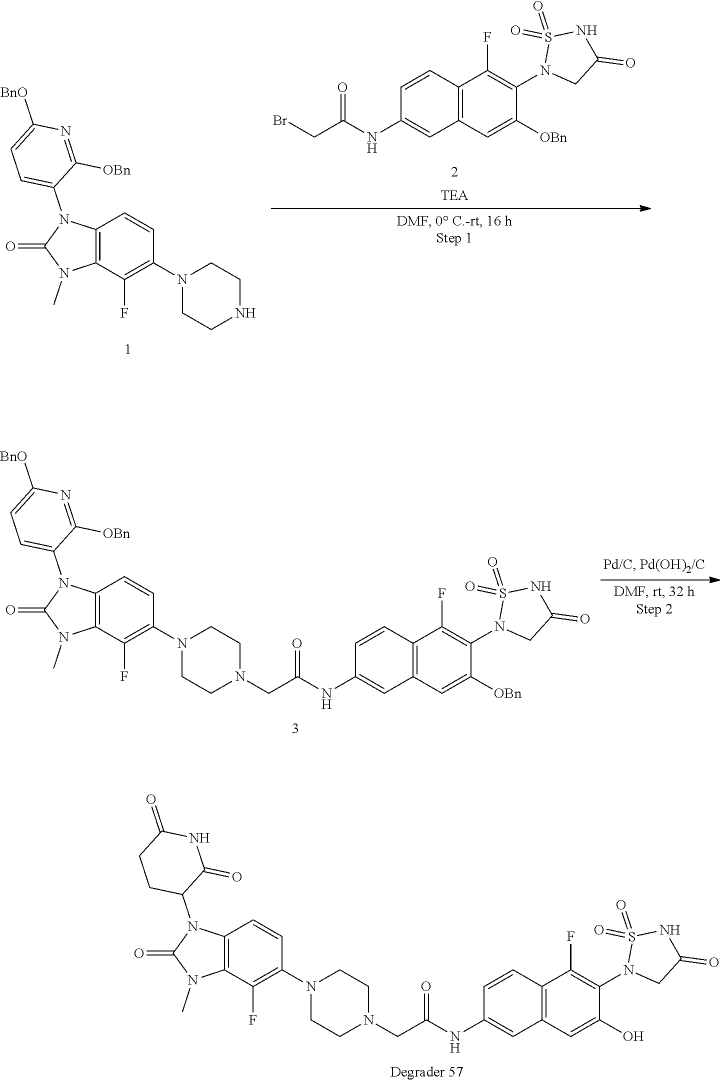 [SMILES: Cn1c(=O)n(-c2ccc(OCc3ccccc3)nc2OCc2ccccc2)c2ccc(N3CCNCC3)c(F)c21 ; O=C(CBr)Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(N3CCN(CC(=O)Nc4ccc5c(F)c(N6CC(=O)NS6(=O)=O)c(OCc6ccccc6)cc5c4)CC3)c(F)c21 ; Cn1c(=O)n(C2CCC(=O)NC2=O)c2ccc(N3CCN(CC(=O)Nc4ccc5c(F)c(N6CC(=O)NS6(=O)=O)c(O)cc5c4)CC3)c(F)c21]

### Step 1: N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-4-fluoro-3-methyl-2-oxo-2,3-dihydro-1H-benzo[d]imidazol-5-yl)piperazin-1-yl)acetamide (3)

To a solution of 1-(2,6-bis(benzyloxy)pyridin-3-yl)-4-fluoro-3-methyl-5-(piperazin-1-yl)-1H-benzo[d]imidazol-2 (3H)-one (1, 200 mg, 347.19 μmol, 1 eq, HCl salt) in DMF (2 mL) were added TEA (87.83 mg, 867.97 μmol, 120.98 μL, 2.5 eq) and N-(7-(benzyloxy)-6-(1,1-dioxido-4-oxo-1,2,5-thiadiazolidin-2-yl)-5-fluoronaphthalen-2-yl)-2-bromoacetamide (2, 190.42 mg, 364.55 μmol, 1.05 eq) at 0° C. The mixture was stirred at 20° C. for 16 h. The mixture was filtered and concentrated in vacuum. The residue was purified by reversed phase flash (flow: 60 mL/min; gradient: from 0-60% MeCN in water (0.1% NH₃H₂O) over 10 min; column: 80 g Flash Column Welch Ultimate XB_C18 20-40 μm; 120 Å) to afford N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-4-fluoro-3-methyl-2-oxo-2,3-dihydro-1H-benzo[d]imidazol-5-yl)piperazin-1-yl)acetamide (3, 150 mg, 136.08 μmol, 39% yield) as a white solid.

LCMS (ESI): m/z 981.4 [M+H]⁺

### Step 2: N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-4-fluoro-3-methyl-2-oxo-2,3-dihydro-1H-benzo[d]imidazol-5-yl)piperazin-1-yl)acetamide (Degrader 57)

To a solution of N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-4-fluoro-3-methyl-2-oxo-2,3-dihydro-1H-benzo[d]imidazol-5-yl)piperazin-1-yl)acetamide (3, 120 mg, 122.32 μmol, 1 eq) in DMF (12 mL) were added Pd/C (120 mg, 10% purity) and Pd(OH)₂/C (120 mg, 10% purity) under N₂ atmosphere. The suspension was degassed and purged with H₂ 3 times. The mixture was stirred under H₂ (15 psi) at 20° C. for 16 h. The mixture was filtered and added Pd/C (120 mg, 10% purity) and Pd(OH)₂/C (120 mg, 10% purity). The mixture was stirred under H₂ (15 psi) at 20° C. for 16 h. The mixture was filtered and concentrated in vacuum. The residue was purified by prep-HPLC (flow: 25 mL/min; gradient: from 14-44% MeCN in water (0.1% TFA) over 9 min; column: 3_Phenomenex Luna C18 75×30 mm×3 um) to afford N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-4-fluoro-3-methyl-2-oxo-2,3-dihydro-1H-benzo[d]imidazol-5-yl)piperazin-1-yl)acetamide (Degrader 57, 47.32 mg, 56.67 μmol, 46% yield, TFA salt) as a pink solid.

LCMS (ESI): m/z 713.5 [M+H]⁺

¹H NMR (400 MHz, d₆-DMSO) δ 11.13 (s, 1H), 10.83 (s, 1H), 10.44-9.89 (m, 2H), 8.13 (s, 1H), 7.92 (d, J=8.8 Hz, 1H), 7.49 (dd, J=1.6, 9.2 Hz, 1H), 7.01 (s, 1H), 6.95-6.90 (m, 1H), 6.83 (t, J=8.4 Hz, 1H), 5.37 (dd, J=5.2, 12.8 Hz, 1H), 4.32 (s, 2H), 4.19 (s, 2H), 3.79 (dd, J=4.8, 7.2 Hz, 2H), 3.49 (s, 3H), 3.43-3.09 (m, 6H), 2.95-2.86 (m, 1H), 2.76-2.64 (m, 2H), 2.07-1.98 (m, 1H).

### 1-[2-[1-[1-(2,6-dioxo-3-piperidyl)-6-fluoro-3-methyl-2-oxo-benzimidazol-5-yl]-4-piperidyl]ethyl]-3-[5-fluoro-7-hydroxy-6-(1,1,4-trioxo-1,2,5-thiadiazolidin-2-yl)-2-naphthyl]urea (Degrader 58)

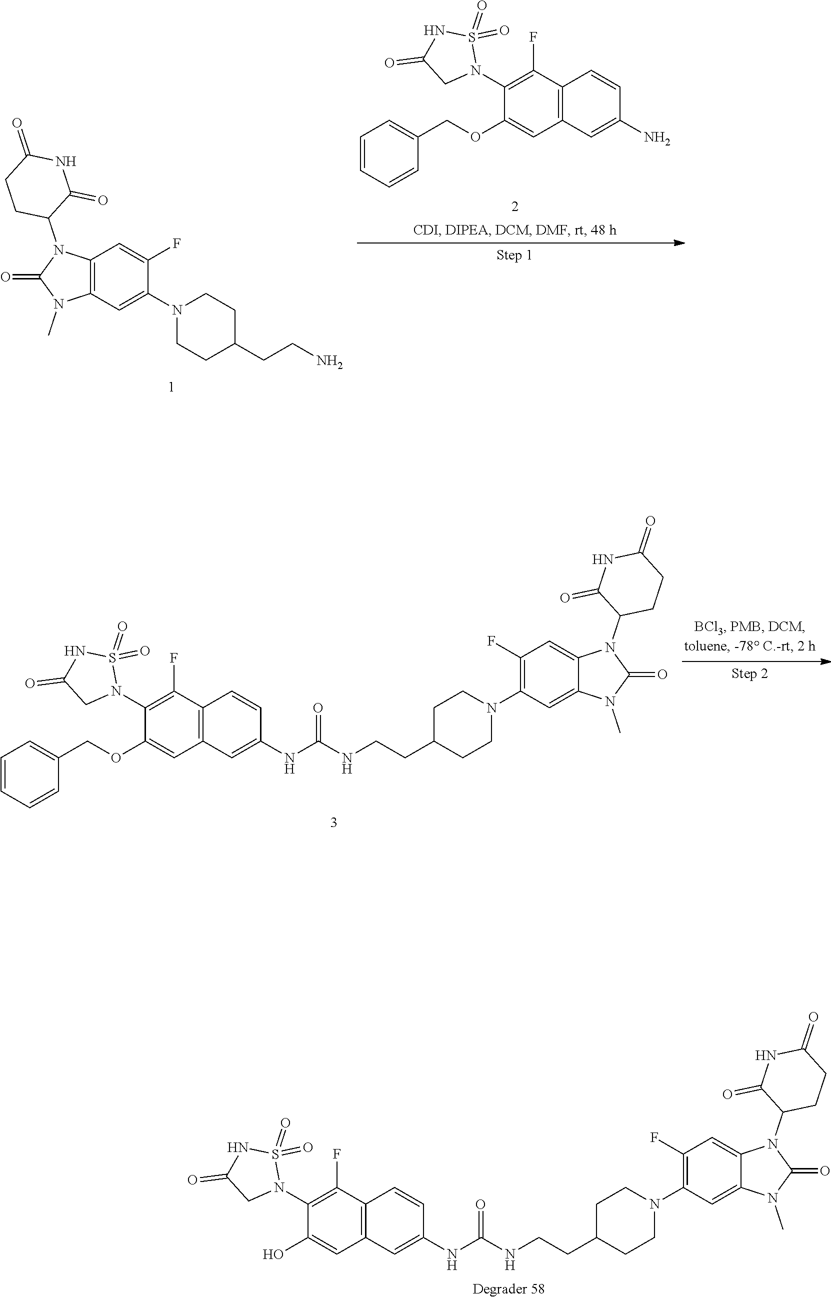 [SMILES: Cn1c(=O)n(C2CCC(=O)NC2=O)c2cc(F)c(N3CCC(CCN)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2cc(F)c(N3CCC(CCNC(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2cc(F)c(N3CCC(CCNC(=O)Nc4ccc5c(F)c(N6CC(=O)NS6(=O)=O)c(O)cc5c4)CC3)cc21]

### Step 1: 1-[7-benzyloxy-5-fluoro-6-(1,1,4-trioxo-1,2,5-thiadiazolidin-2-yl)-2-naphthyl]-3-[2-[1-[1-(2,6-dioxo-3-piperidyl)-6-fluoro-3-methyl-2-oxo-benzimidazol-5-yl]-4-piperidyl]ethyl]urea (3)

Into a 50 mL single neck round-bottom flask containing a well-stirred solution of 5-(6-amino-3-benzyloxy-1-fluoro-2-naphthyl)-1,1-dioxo-1,2,5-thiadiazolidin-3-one (2, 120 mg, 232.35 μmol, TFA salt) in anhydrous DCM (0.7 mL) and DMF (0.2 mL) were added DIPEA (214.50 mg, 1.66 mmol, 289.08 μL) and CDI (161.47 mg, 995.78 μmol) at room temperature. The mixture stirred for 2 h at room temperature. Then, a solution of 3-[5-[4-(2-aminoethyl)-1-piperidyl]-6-fluoro-3-methyl-2-oxo-benzimidazol-1-yl]piperidine-2,6-dione (1, 173.50 mg, 331.93 μmol, TFA salt) and DIPEA (214.50 mg, 1.66 mmol, 289.08 μL) in DMF (0.2 mL) was added and the reaction mixture was stirred at room temperature. After 48 h, the solvent was removed to dryness and the residue was treated with 1.5N HCl (30 mL). The precipitate was filtered and dried under reduced pressure to get crude 1-[7-benzyloxy-5-fluoro-6-(1,1,4-trioxo-1,2,5-thiadiazolidin-2-yl)-2-naphthyl]-3-[2-[1-[1-(2,6-dioxo-3-piperidyl)-6-fluoro-3-methyl-2-oxo-benzimidazol-5-yl]-4-piperidyl]ethyl]urea (3, 170 mg, 141.18 μmol, 43% yield) as brown solid. LCMS (ES+): m/z 832.0 [M+H]⁺

### Step 2: 1-[2-[1-[1-(2,6-dioxo-3-piperidyl)-6-fluoro-3-methyl-2-oxo-benzimidazol-5-yl]-4-piperidyl]ethyl]-3-[5-fluoro-7-hydroxy-6-(1,1,4-trioxo-1,2,5-thiadiazolidin-2-yl)-2-naphthyl]urea (Degrader 58)

Into a 50 mL single neck round-bottom flask containing a well-stirred solution of 1-[7-benzyloxy-5-fluoro-6-(1,1,4-trioxo-1,2,5-thiadiazolidin-2-yl)-2-naphthyl]-3-[2-[1-[1-(2,6-dioxo-3-piperidyl)-6-fluoro-3-methyl-2-oxo-benzimidazol-5-yl]-4-piperidyl]ethyl]urea (3, 170 mg, 141.18 μmol), pentamethylbenzene (125.58 mg, 847.08 μmol) in DCM (3 mL) and toluene (3 mL) was added a 1.0 M solution of boron trichloride in methylene chloride (330.84 mg, 2.82 mmol, 2.82 mL) at −78° C. The reaction mixture was stirred at room temperature for 2 h. The reaction mixture was quenched with 5% MeOH in DCM (2 mL) at −78° C. and the volatiles were removed under reduced pressure. The crude compound was washed with MTBE (60 mL) and purified by reverse phase column chromatography [Silicycle C18 column; Mobile phase A: 0.1% TFA in water; Mobile phase B: MeCN] to get 1-[2-[1-[1-(2,6-dioxo-3-piperidyl)-6-fluoro-3-methyl-2-oxo-benzimidazol-5-yl]-4-piperidyl]ethyl]-3-[5-fluoro-7-hydroxy-6-(1,1,4-trioxo-1,2,5-thiadiazolidin-2-yl)-2-naphthyl]urea (Degrader 58, 45 mg, 51.19 μmol, 36% yield, TFA salt) as an off-white solid.

LCMS (ES+): m/z 741.0 [M+H]⁺

¹HNMR (400 MHz, DMSO-d6): δ 11.07 (s, 1H), 10.20 (bs, 1H), 8.75 (s, 1H), 7.91 (s, 1H), 7.78 (d, J=9.20 Hz, 1H), 7.28 (dd, J=2.00, 9.00 Hz, 1H), 7.12 (d, J=12.00 Hz, 1H), 6.99-6.96 (m, 1H), 6.90 (s, 1H), 6.29 (bs, 1H), 5.33-5.29 (m, 1H), 4.35 (s, 2H), 3.56 (s, 3H), 3.36-3.20 (m, 4H), 2.87-2.83 (m, 1H), 2.74-2.55 (m, 5H), 2.00-1.97 (m, 1H), 1.84-1.81 (m, 2H), 1.53-1.48 (m, 3H), 1.42-1.37 (m, 2H).

### N-[2-[4-[1-(2,6-dioxo-3-piperidyl)-3-methyl-2-oxo-benzimidazol-5-yl]-3,3-difluoro-1-piperidyl]ethyl]-5-fluoro-7-hydroxy-6-(1,1,4-trioxo-1,2,5-thiadiazolidin-2-yl)naphthalene-2-carboxamide (Degrader 59)

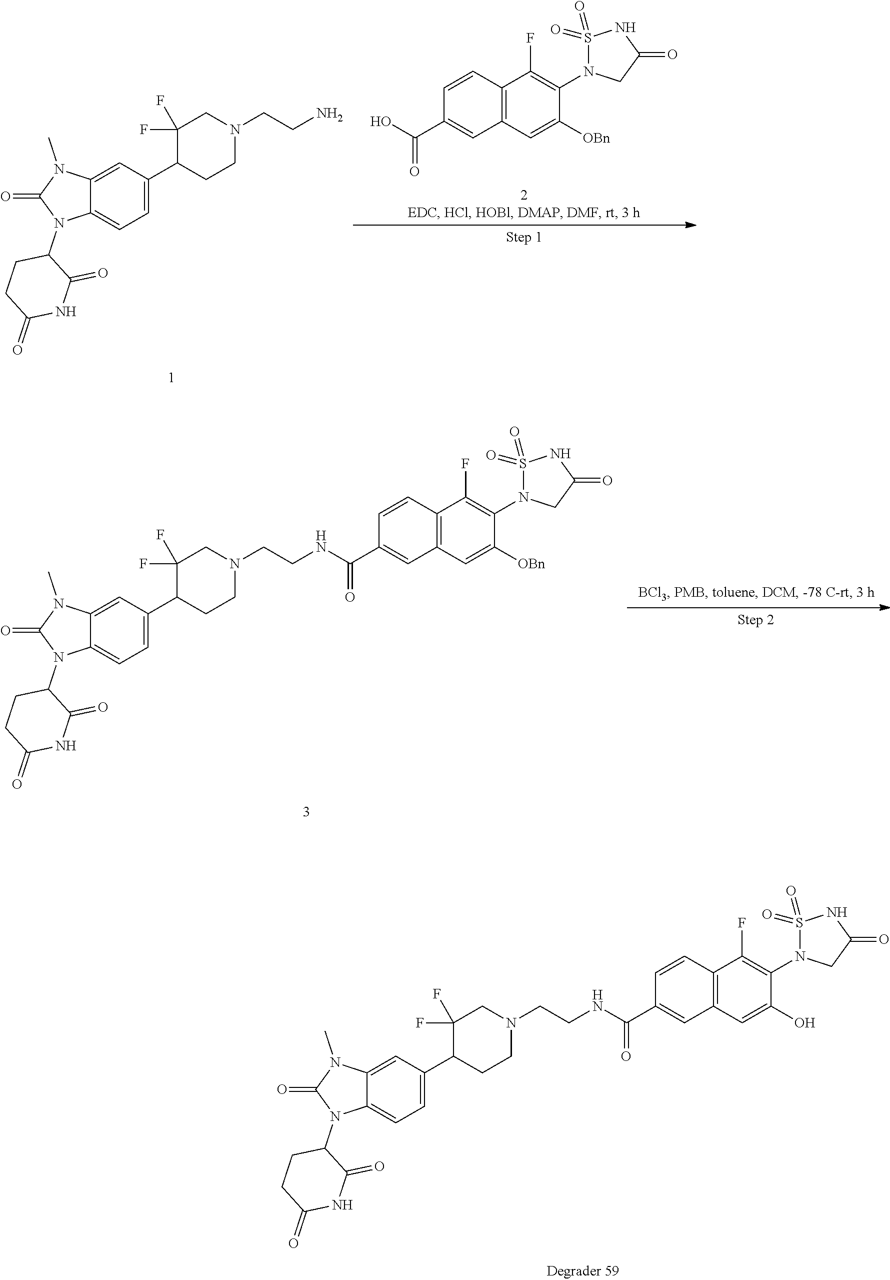 [SMILES: Cn1c(=O)n(C2CCC(=O)NC2=O)c2ccc(C3CCN(CCN)CC3(F)F)cc21 ; O=C1CN(c2c(OCc3ccccc3)cc3cc(C(=O)O)ccc3c2F)S(=O)(=O)N1 ; Cn1c(=O)n(C2CCC(=O)NC2=O)c2ccc(C3CCN(CCNC(=O)c4ccc5c(F)c(N6CC(=O)NS6(=O)=O)c(OCc6ccccc6)cc5c4)CC3(F)F)cc21 ; Cn1c(=O)n(C2CCC(=O)NC2=O)c2ccc(C3CCN(CCNC(=O)c4ccc5c(F)c(N6CC(=O)NS6(=O)=O)c(O)cc5c4)CC3(F)F)cc21]

### Step 1: 7-benzyloxy-N-[2-[4-[1-(2,6-dioxo-3-piperidyl)-3-methyl-2-oxo-benzimidazol-5-yl]-3,3-difluoro-1-piperidyl]ethyl]-5-fluoro-6-(1,1,4-trioxo-1,2,5-thiadiazolidin-2-yl)naphthalene-2-carboxamide (3)

Into a 50 mL single-neck round-bottom flask containing a well-stirred solution of 3-[5-[1-(2-aminoethyl)-3,3-difluoro-4-piperidyl]-3-methyl-2-oxo-benzimidazol-1-yl]piperidine-2,6-dione (1, 130 mg, 235.98 μmol, TFA salt) and 7-benzyloxy-5-fluoro-6-(1,1,4-trioxo-1,2,5-thiadiazolidin-2-yl)naphthalene-2-carboxylic acid (2, 133.37 mg, 306.78 μmol) in anhydrous DMF (3 mL) were added EDC·HCl (180.02 mg, 943.93 μmol), HOBT (63.77 mg, 471.97 μmol) and DMAP (201.81 mg, 1.65 mmol). After 3 h, the reaction mixture was quenched with 50 mL of aqueous 1.5 N HCl solution. The precipitate was filtered and filtrate was concentrated under reduced pressure to get 7-benzyloxy-N-[2-[4-[1-(2,6-dioxo-3-piperidyl)-3-methyl-2-oxo-benzimidazol-5-yl]-3,3-difluoro-1-piperidyl]ethyl]-5-fluoro-6-(1,1,4-trioxo-1,2,5-thiadiazolidin-2-yl)naphthalene-2-carboxamide (3, 200 mg, 204.90 μmol, 87% yield, HCl salt) as a colorless solid.

LCMS (ES+): m/z 834.0 [M+H]⁺

### Step 2: N-[2-[4-[1-(2,6-dioxo-3-piperidyl)-3-methyl-2-oxo-benzimidazol-5-yl]-3,3-difluoro-1-piperidyl]ethyl]-5-fluoro-7-hydroxy-6-(1,1,4-trioxo-1,2,5-thiadiazolidin-2-yl)naphthalene-2-carboxamide (Degrader 59)

Into a 50 mL single neck round-bottom flask containing a well-stirred solution of 7-benzyloxy-N-[2-[4-[1-(2,6-dioxo-3-piperidyl)-3-methyl-2-oxo-benzimidazol-5-yl]-3,3-difluoro-1-piperidyl]ethyl]-5-fluoro-6-(1,1,4-trioxo-1,2,5-thiadiazolidin-2-yl)naphthalene-2-carboxamide (3, 200 mg, 204.90 μmol, HCl salt), pentamethylbenzene (151.87 mg, 1.02 mmol) in anhydrous DCM (2 mL) and toluene (2 mL) was added a 1.0 M boron trichloride in DCM (480.16 mg, 4.10 mmol, 4.1 mL) at −78° C. The reaction mixture was stirred at room temperature. After 3 h, the mixture was quenched with 5% MeOH in DCM (5 mL) at −78° C. and concentrated under reduced pressure to get the crude compound. The residue was washed with MTBE (50 mL) and purified by reverse phase column chromatography [Silicycle C18 column; Mobile phase A: 0.1% formic acid in water; Mobile phase B: MeCN] to get N-[2-[4-[1-(2,6-dioxo-3-piperidyl)-3-methyl-2-oxo-benzimidazol-5-yl]-3,3-difluoro-1-piperidyl]ethyl]-5-fluoro-7-hydroxy-6-(1,1,4-trioxo-1,2,5-thiadiazolidin-2-yl)naphthalene-2-carboxamide (Degrader 59, 145 mg, 192.18 μmol, 94% yield) as a colorless solid.

LCMS (ES+): m/z 744.0 [M+H]⁺

¹HNMR (400 MHz, DMSO-d6): δ 11.11 (s, 1H), 10.27 (s, 1H), 8.92 (s, 1H), 8.29 (s, 1H), 8.01 (d, J=8.80 Hz, 1H), 7.80 (dd, J=1.20, 8.80 Hz, 1H), 7.21 (s, 1H), 7.12-7.11 (m, 2H), 7.00 (d, J=8.00 Hz, 1H), 5.41-5.36 (m, 1H), 4.21 (s, 2H), 3.67-3.53 (m, 3H), 2.92-2.88 (m, 1H), 2.68-2.61 (m, 2H), 2.25-2.39 (m, 1H) 2.04-2.02 (m, 2H).

Note: Several protons in the aliphatic region merge into DMSO peak. The proton count was confirmed by D2O exchange data.

### 2-[4-[1-(2,6-dioxo-3-piperidyl)-3-methyl-2-oxo-imidazo[4,5-b]pyridin-5-yl]phenyl]-N-[5-fluoro-7-hydroxy-6-(1,1,4-trioxo-1,2,5-thiadiazolidin-2-yl)-2-naphthyl]acetamide (Degrader 60)

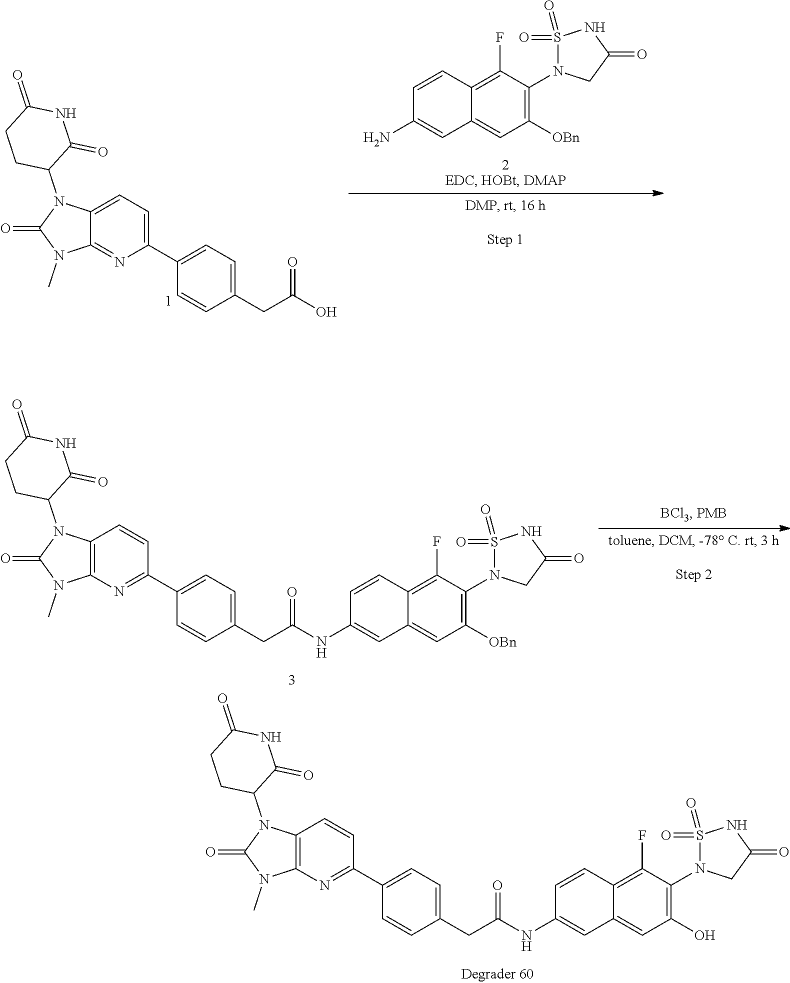 [SMILES: Cn1c(=O)n(C2CCC(=O)NC2=O)c2ccc(-c3ccc(CC(=O)O)cc3)nc21 ; Nc1ccc2c(F)c(N3CC(=O)NS3(=O)=O)c(OCc3ccccc3)cc2c1 ; Cn1c(=O)n(C2CCC(=O)NC2=O)c2ccc(-c3ccc(CC(=O)Nc4ccc5c(F)c(N6CC(=O)NS6(=O)=O)c(OCc6ccccc6)cc5c4)cc3)nc21 ; Cn1c(=O)n(C2CCC(=O)NC2=O)c2ccc(-c3ccc(CC(=O)Nc4ccc5c(F)c(N6CC(=O)NS6(=O)=O)c(O)cc5c4)cc3)nc21]

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-imidazo[4,5-b]pyridin-5-yl]phenyl]acetamide (3)

Into 25 mL two-neck round-bottom flask containing a well-stirred solution of 2-[4-[1-(2,6-dioxo-3-piperidyl)-3-methyl-2-oxo-imidazo[4,5-b]pyridin-5-yl]phenyl]acetic acid (1, 0.2 g, 349.68 μmol, formic acid salt) in DMF (5 mL) were added EDC·HCl (201.10 mg, 1.05 mmol), HOBt (94.50 mg, 699.36 μmol) and DMAP (256.32 mg, 2.10 mmol) at room temperature. The resulting mixture was stirred at room temperature for 1 h. Afterwards, 5-(6-amino-3-benzyloxy-1-fluoro-2-naphthyl)-1,1-dioxo-1,2,5-thiadiazolidin-3-one (2, 154.33 mg, 326.79 μmol, TFA salt) was added and stirring was continued at room temperature for 16 h. The reaction mixture was evaporated under reduced pressure and then diluted with cold water (10 mL) to precipitate a solid that was filtered on a Buchner funnel and dried to afford the crude N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-imidazo[4,5-b]pyridin-5-yl]phenyl]acetamide (3, 0.38 g, 186.05 μmol, 53% yield) as a brown solid.

LCMS (ES+): m/z 778.0 [M+H]⁺

### Step 2: 2-[4-[1-(2,6-dioxo-3-piperidyl)-3-methyl-2-oxo-imidazo[4,5-b]pyridin-5-yl]phenyl]-N-[5-fluoro-7-hydroxy-6-(1,1,4-trioxo-1,2,5-thiadiazolidin-2-yl)-2-naphthyl]acetamide (Degrader 60)

Into a 25 mL two-neck round-bottom flask containing a well-stirred solution of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-imidazo[4,5-b]pyridin-5-yl]phenyl]acetamide (3, 380 mg, 185.66 μmol) in a 1:1 mixture of anhydrous DCM (5 mL) and toluene (5 mL) was added pentamethylbenzene (137.61 mg, 928.29 μmol) at ambient temperature under nitrogen atmosphere. The reaction mixture was then cooled to −78° C. and boron trichloride (1.0 M solution in DCM, 4.64 mmol, 4.6 mL) was added dropwise. The resulting mixture was stirred at room temperature for 3 h. The reaction mixture was again cooled to −78° C. and quenched with 5% MeOH/DCM (4 mL). The reaction mixture was concentrated under reduced pressure to get the crude material that was subjected to purification by a reverse-phase preparative HPLC [Column; SUNFIRE C18 (19×150) mm, 5 Micron; Mobile Phase A: 0.1% TFA in water and Mobile Phase B: CH₃CN] to afford 2-[4-[1-(2,6-dioxo-3-piperidyl)-3-methyl-2-oxo-imidazo[4,5-b]pyridin-5-yl]phenyl]-N-[5-fluoro-7-hydroxy-6-(1,1,4-trioxo-1,2,5-thiadiazolidin-2-yl)-2-naphthyl]acetamide (Degrader 60, 32 mg, 36.36 μmol, 20% yield, TFA salt) as an off-white powder.

LCMS (ES−): m/z 686.0 [M−H]⁻

¹H-NMR (400 MHz, DMSO-d6): δ 11.17 (s, 1H), 10.46 (s, 1H), 10.31-10.41 (brs, 1H), 8.19 (s, 1H), 8.04 (d, J=8.40 Hz, 2H), 7.87 (d, J=9.20 Hz, 1H), 7.66 (d, J=8.40 Hz, 1H), 7.58 (d, J=8.40 Hz, 1H), 7.47 (d, J=8.40 Hz, 3H), 6.96 (s, 1H), 5.46 (dd, J=5.20, 13.00 Hz, 1H), 4.38 (s, 2H), 4.03 (s, 2H), 3.44 (s, 3H), 2.95-2.88 (m, 1H), 2.81-2.60 (m, 2H), 2.15-2.05 (m, 1H).

### N-[2-[4-[3-(2,6-dioxo-3-piperidyl)-1-methyl-indazol-6-yl]-3,3-difluoro-1-piperidyl]ethyl]-5-fluoro-7-hydroxy-6-(1,1,4-trioxo-1,2,5-thiadiazolidin-2-yl)naphthalene-2-carboxamide (Degrader 61)

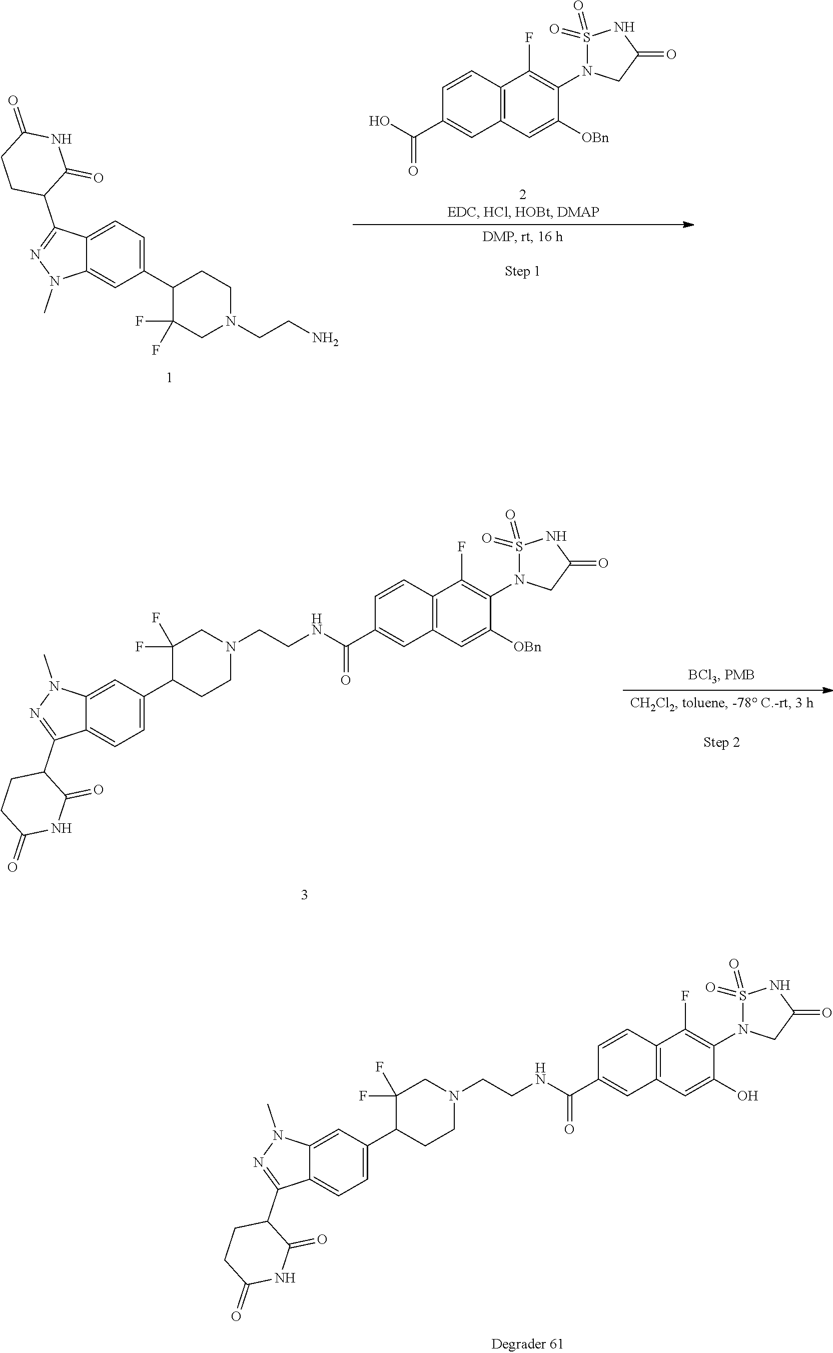 [SMILES: Cn1nc(C2CCC(=O)NC2=O)c2ccc(C3CCN(CCN)CC3(F)F)cc21 ; O=C1CN(c2c(OCc3ccccc3)cc3cc(C(=O)O)ccc3c2F)S(=O)(=O)N1 ; Cn1nc(C2CCC(=O)NC2=O)c2ccc(C3CCN(CCNC(=O)c4ccc5c(F)c(N6CC(=O)NS6(=O)=O)c(OCc6ccccc6)cc5c4)CC3(F)F)cc21 ; Cn1nc(C2CCC(=O)NC2=O)c2ccc(C3CCN(CCNC(=O)c4ccc5c(F)c(N6CC(=O)NS6(=O)=O)c(O)cc5c4)CC3(F)F)cc21]

### Step 1: 7-benzyloxy-N-[2-[4-[3-(2,6-dioxo-3-piperidyl)-1-methyl-indazol-6-yl]-3,3-difluoro-1-piperidyl]ethyl]-5-fluoro-6-(1,1,4-trioxo-1,2,5-thiadiazolidin-2-yl)naphthalene-2-carboxamide (3)

Into a 20 mL screw-capped vial containing a well-stirred solution of 3-[6-[1-(2-aminoethyl)-3,3-difluoro-4-piperidyl]-1-methyl-indazol-3-yl]piperidine-2,6-dione (1, 110 mg, 243.65 μmol, formic acid salt) and 7-benzyloxy-5-fluoro-6-(1,1,4-trioxo-1,2,5-thiadiazolidin-2-yl)naphthalene-2-carboxylic acid (2, 136.33 mg, 316.75 μmol) in DMF (3 mL) were added EDC (186.83 mg, 974.60 μmol), HOBt (65.84 mg, 487.30 μmol) and DMAP (208.37 mg, 1.71 mmol) at room temperature and the resulting mixture was stirred at room temperature for 16 h. The reaction mixture was concentrated under reduced pressure and treated with ice-cold water to obtain a solid that was filtered and dried under vacuum to afford 7-benzyloxy-N-[2-[4-[3-(2,6-dioxo-3-piperidyl)-1-methyl-indazol-6-yl]-3,3-difluoro-1-piperidyl]ethyl]-5-fluoro-6-(1,1,4-trioxo-1,2,5-thiadiazolidin-2-yl)naphthalene-2-carboxamide (3, 120 mg, 66.31 μmol, 27% yield) as an off-white solid.

LCMS (ES+): m/z 818.9 [M+H]⁺

### Step 2: N-[2-[4-[3-(2,6-dioxo-3-piperidyl)-1-methyl-indazol-6-yl]-3,3-difluoro-1-piperidyl]ethyl]-5-fluoro-7-hydroxy-6-(1,1,4-trioxo-1,2,5-thiadiazolidin-2-yl)naphthalene-2-carboxamide (Degrader 61)

Into a 50 mL single-neck round-bottom flask containing a well-stirred solution of 7-benzyloxy-N-[2-[4-[3-(2,6-dioxo-3-piperidyl)-1-methyl-indazol-6-yl]-3,3-difluoro-1-piperidyl]ethyl]-5-fluoro-6-(1,1,4-trioxo-1,2,5-thiadiazolidin-2-yl)naphthalene-2-carboxamide (3, 120 mg, 146.73 μmol) and pentamethylbenzene (108.76 mg, 733.65 μmol, 118.61 μL) in anhydrous toluene (3 mL) and DCM (3 mL) was added dropwise BCl₃ (1.0 M solution in DCM, 293 mmol, 2.93 mL) at −78° C. The resulting mixture was stirred at rt for 3 h and quenched with 5 mL of 5% MeOH in DCM. The solvent was evaporated to get the crude material that was purified by reverse-phase preparative HPLC [Column: X SELECT C18 (150×19) mm, 5 μm; Mobile Phase A: 0.1% HCOOH in water and Mobile Phase B: MeCN] to afford N-[2-[4-[3-(2,6-dioxo-3-piperidyl)-1-methyl-indazol-6-yl]-3,3-difluoro-1-piperidyl]ethyl]-5-fluoro-7-hydroxy-6-(1,1,4-trioxo-1,2,5-thiadiazolidin-2-yl)naphthalene-2-carboxamide (Degrader 61, 36 mg, 44.24 μmol, 30% yield, formic acid salt) as an off-white solid

¹H-NMR (400 MHz, DMSO-d6): δ 10.91 (s, 1H), 10.23 (s, 1H), 8.85 (brs, 1H), 8.28 (s, 1H), 7.99 (d, J=8.40 Hz, 1H), 7.80 (dd, J=1.20, 8.80 Hz, 1H), 7.69 (d, J=8.40 Hz, 1H), 7.54 (s, 1H), 7.20 (s, 1H), 7.09 (d, J=8.40 Hz, 1H), 4.37 (dd, J=5.20, 10.00 Hz, 1H), 4.17 (s, 2H), 4.01 (s, 3H), 3.72-3.55 (m, 3H), 2.75-2.55 (m, 3H), 2.40-2.34 (m, 2H), 2.22-2.15 (m, 1H), 2.10-2.02 (m, 1H).

[Note: 5 aliphatic protons attached to the nitrogen are eclipsed by the moisture in the solvent]

LCMS (ES−): m/z 726.0 [M−H]⁻

### 1-(6-(1,1-dioxido-4-oxo-1,2,5-thiadiazolidin-2-yl)-5-fluoro-7-hydroxynaphthalen-2-yl)-3-(2-(1-(1-(2,6-dioxopiperidin-3-yl)-3-methyl-2-oxo-2,3-dihydro-1H-benzo[d]imidazol-4-yl)piperidin-4-yl)ethyl)urea (Degrader 62)

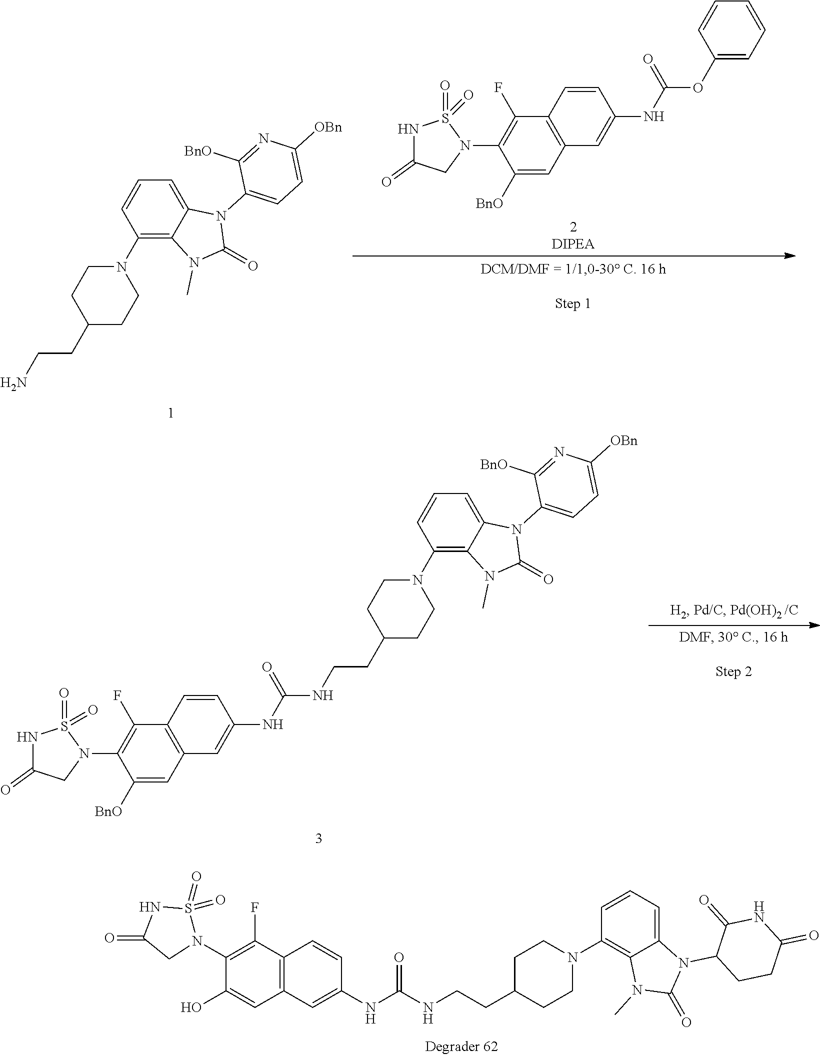 [SMILES: Cn1c(=O)n(-c2ccc(OCc3ccccc3)nc2OCc2ccccc2)c2cccc(N3CCC(CCN)CC3)c21 ; O=C1CN(c2c(OCc3ccccc3)cc3cc(NC(=O)Oc4ccccc4)ccc3c2F)S(=O)(=O)N1 ; Cn1c(=O)n(-c2ccc(OCc3ccccc3)nc2OCc2ccccc2)c2cccc(N3CCC(CCNC(=O)Nc4ccc5c(F)c(N6CC(=O)NS6(=O)=O)c(OCc6ccccc6)cc5c4)CC3)c21 ; Cn1c(=O)n(C2CCC(=O)NC2=O)c2cccc(N3CCC(CCNC(=O)Nc4ccc5c(F)c(N6CC(=O)NS6(=O)=O)c(O)cc5c4)CC3)c21]

### Step 1: 1-(7-(benzyloxy)-6-(1,1-dioxido-4-oxo-1,2,5-thiadiazolidin-2-yl)-5-fluoronaphthalen-2-yl)-3-(2-(1-(1-(2,6-bis(benzyloxy)pyridin-3-yl)-3-methyl-2-oxo-2,3-dihydro-1H-benzo[d]imidazol-4-yl)piperidin-4-yl)ethyl)urea (3)

To a solution of 4-(4-(2-aminoethyl)piperidin-1-yl)-1-(2,6-bis(benzyloxy)pyridin-3-yl)-3-methyl-1H-benzo[d]imidazol-2 (3H)-one (1, 130 mg, 230.62 μmol, 1 eq) in DCM (2 mL) and DMF (2 mL) were added DIPEA (149.03 mg, 1.15 mmol, 200.85 μL, 5 eq) and phenyl (7-(benzyloxy)-6-(1,1-dioxido-4-oxo-1,2,5-thiadiazolidin-2-yl)-5-fluoronaphthalen-2-yl)carbamate (2, 120.27 mg, 230.62 μmol, 1 eq) at 0° C. The mixture was stirred at 30° C. for 16 h. The mixture was filtered and concentrated in vacuum. The residue was purified by prep-HPLC (flow: 25 mL/min; gradient: from 35-65% MeCN in water (0.05% ammonia hydroxide) over 12 min; column: Waters Xbridge 150×25 mm×5 um) to afford 1-(7-(benzyloxy)-6-(1,1-dioxido-4-oxo-1,2,5-thiadiazolidin-2-yl)-5-fluoronaphthalen-2-yl)-3-(2-(1-(1-(2,6-bis(benzyloxy)pyridin-3-yl)-3-methyl-2-oxo-2,3-dihydro-1H-benzo[d]imidazol-4-yl)piperidin-4-yl)ethyl)urea (3, 150 mg, 149.83 μmol, 65% yield) as a white solid.

LCMS (ESI): m/z 991.5 [M+H]⁺

### Step 2: 1-(6-(1,1-dioxido-4-oxo-1,2,5-thiadiazolidin-2-yl)-5-fluoro-7-hydroxynaphthalen-2-yl)-3-(2-(1-(1-(2,6-dioxopiperidin-3-yl)-3-methyl-2-oxo-2,3-dihydro-1H-benzo[d]imidazol-4-yl)piperidin-4-yl)ethyl)urea (Degrader 62)

To a solution of 1-(7-(benzyloxy)-6-(1,1-dioxido-4-oxo-1,2,5-thiadiazolidin-2-yl)-5-fluoronaphthalen-2-yl)-3-(2-(1-(1-(2,6-bis(benzyloxy)pyridin-3-yl)-3-methyl-2-oxo-2,3-dihydro-1H-benzo[d]imidazol-4-yl)piperidin-4-yl)ethyl)urea (3, 150 mg, 151.35 μmol, 1 eq) in DMF (15 mL) were added Pd/C (150 mg, 10% purity) and Pd(OH)₂/C (150 mg, 10% purity) under N₂ atmosphere. The suspension was degassed and purged with H₂ 3 times. The mixture was stirred under H₂ (15 psi) at 30° C. for 16 h. The mixture was filtered and concentrated in vacuum. The residue was purified by prep-HPLC (flow: 25 mL/min; gradient: from 34-64% MeCN in water (0.1% TFA) over 9 min; column: 3_Phenomenex Luna C18 75×30 mm×3 um) to afford 1-(6-(1,1-dioxido-4-oxo-1,2,5-thiadiazolidin-2-yl)-5-fluoro-7-hydroxynaphthalen-2-yl)-3-(2-(1-(1-(2,6-dioxopiperidin-3-yl)-3-methyl-2-oxo-2,3-dihydro-1H-benzo[d]imidazol-4-yl)piperidin-4-yl)ethyl)urea (Degrader 62, 70.54 mg, 82.61 μmol, 55% yield, TFA salt) as a pink solid.

LCMS (ESI): m/z 723.1 [M+H]⁺

¹H NMR (400 MHz, d₆-DMSO) δ 11.08 (s, 1H), 10.62-10.33 (m, 1H), 8.74 (s, 1H), 7.93 (s, 1H), 7.78 (d, J=8.8 Hz, 1H), 7.28 (dd, J=1.6, 8.8 Hz, 1H), 7.00-6.94 (m, 1H), 6.93-6.84 (m, 3H), 6.35-6.25 (m, 1H), 5.37-5.32 (dd, J=5.2, 12.4 Hz, 1H), 4.47 (s, 2H), 3.63 (s, 3H), 3.20 (d, J=4.0 Hz, 2H), 3.12 (d, J=9.6 Hz, 2H), 2.95-2.83 (m, 1H), 2.75-2.59 (m, 4H), 2.02-1.96 (m, 1H), 1.81 (d, J=10.8 Hz, 2H), 1.52-1.36 (m, 5H).

### (2R)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)-3-hydroxypropanamide (Degrader 63)

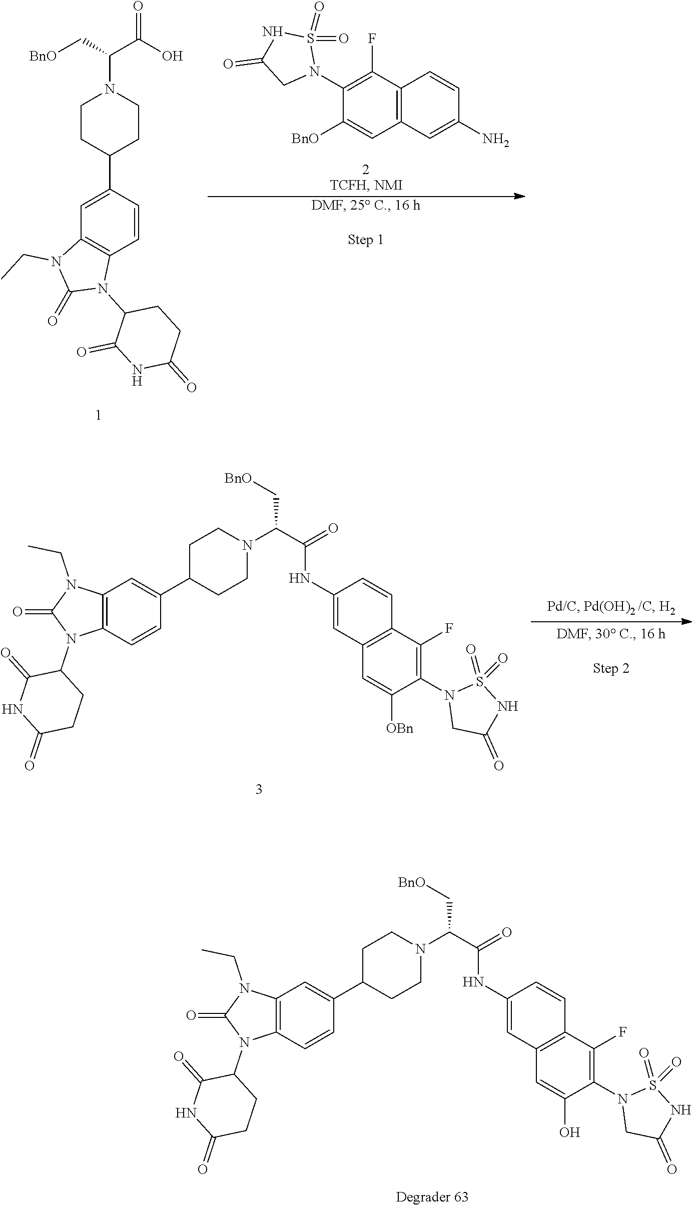 [SMILES: CCn1c(=O)n(C2CCC(=O)NC2=O)c2ccc(C3CCN([C@H](COCc4ccccc4)C(=O)O)CC3)cc21 ; Nc1ccc2c(F)c(N3CC(=O)NS3(=O)=O)c(OCc3ccccc3)cc2c1 ; CCn1c(=O)n(C2CCC(=O)NC2=O)c2ccc(C3CCN([C@H](COCc4ccccc4)C(=O)Nc4ccc5c(F)c(N6CC(=O)NS6(=O)=O)c(OCc6ccccc6)cc5c4)CC3)cc21 ; CCn1c(=O)n(C2CCC(=O)NC2=O)c2ccc(C3CCN([C@H](COCc4ccccc4)C(=O)Nc4ccc5c(F)c(N6CC(=O)NS6(=O)=O)c(O)cc5c4)CC3)cc21]

### Step 1: (2R)-3-(benzyloxy)-N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)propanamide (3)

To a mixture of (2R)-3-(benzyloxy)-2-(4-(1-(2,6-dioxopiperidin-3-yl)-3-ethyl-2-oxo-2,3-dihydro-1H-benzo[d]imidazol-5-yl)piperidin-1-yl)propanoic acid (1, 182 mg, 280.59 μmol, TFA salt, 1 eq), 5-(6-amino-3-(benzyloxy)-1-fluoronaphthalen-2-yl)-1,2,5-thiadiazolidin-3-one 1,1-dioxide (2 144.63 mg, 280.59 μmol, TFA salt, 1 eq) and NMI (115.19 mg, 1.40 mmol, 5 eq) in DMF (10 mL) was added TCFH (196.82 mg, 701.48 μmol, 32.58 μL, 2.5 eq) at 0° C., the mixture was stirred at 20° C. for 16 h. The mixture was concentrated in vacuum. The reaction mixture was purified by prep-HPLC (flow: 25 mL/min; gradient: from 45-75% MeCN in water (0.1% TFA); column: Phenomenex luna C18 150×40 mm×15 um) to afford (2R)-3-(benzyloxy)-N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)propanamide (3, 130 mg, 124.71 μmol, 44% yield, TFA salt) as yellow solid.

LCMS (ESI): m/z 918.4 [M+H]⁺

### Step 2: (2R)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)-3-hydroxypropanamide (Degrader 63)

To a solution of (2R)-3-(benzyloxy)-N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)propanamide (3, 130 mg, 125.97 μmol, TFA salt, 1 eq) in DMF (5 mL) was added Pd/C (100 mg, 10% purity) and Pd(OH)₂/C (100 mg, 10% purity) under H₂ (15 Psi), the mixture was stirred at 30° C. for 16 h under H₂ (15 Psi) atmosphere. The mixture was filtered and concentrated in vacuum. The residue was purified by prep-HPLC (flow: 25 mL/min; gradient: from 55-85% MeCN in water (0.1% TFA); column: Phenomenex Synergi C18 150×25 mm×10 um) to afford (2R)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)-3-hydroxypropanamide (Degrader 63, 59.88 mg, 70.30 μmol, 56% yield, TFA salt) as white solid.

LCMS (ESI): m/z 738.2 [M+H]⁺

¹H NMR (400 MHz, DMSO-d₆) δ 11.09 (s, 1H), 10.81 (br s, 1H), 9.94 (br s, 1H), 9.78-9.59 (m, 1H), 8.15 (s, 1H), 7.92 (d, J=8.8 Hz, 1H), 7.50 (dd, J=1.6, 9.2 Hz, 1H), 7.11-7.06 (m, 2H), 7.02 (s, 1H), 6.94 (br d, J=7.8 Hz, 1H), 5.81 (br s, 1H), 5.35 (br dd, J=5.2, 12.6 Hz, 1H), 4.15-4.11 (m, 4H), 3.89 (q, J=6.8 Hz, 2H), 3.76 (br d, J=7.8 Hz, 1H), 3.42-3.28 (m, 3H), 2.95-2.86 (m, 2H), 2.72-2.62 (m, 2H), 2.29-1.97 (m, 6H), 1.24 (t, J=7.2 Hz, 3H).

### (2S)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)-3-hydroxypropanamide (Degrader 64)

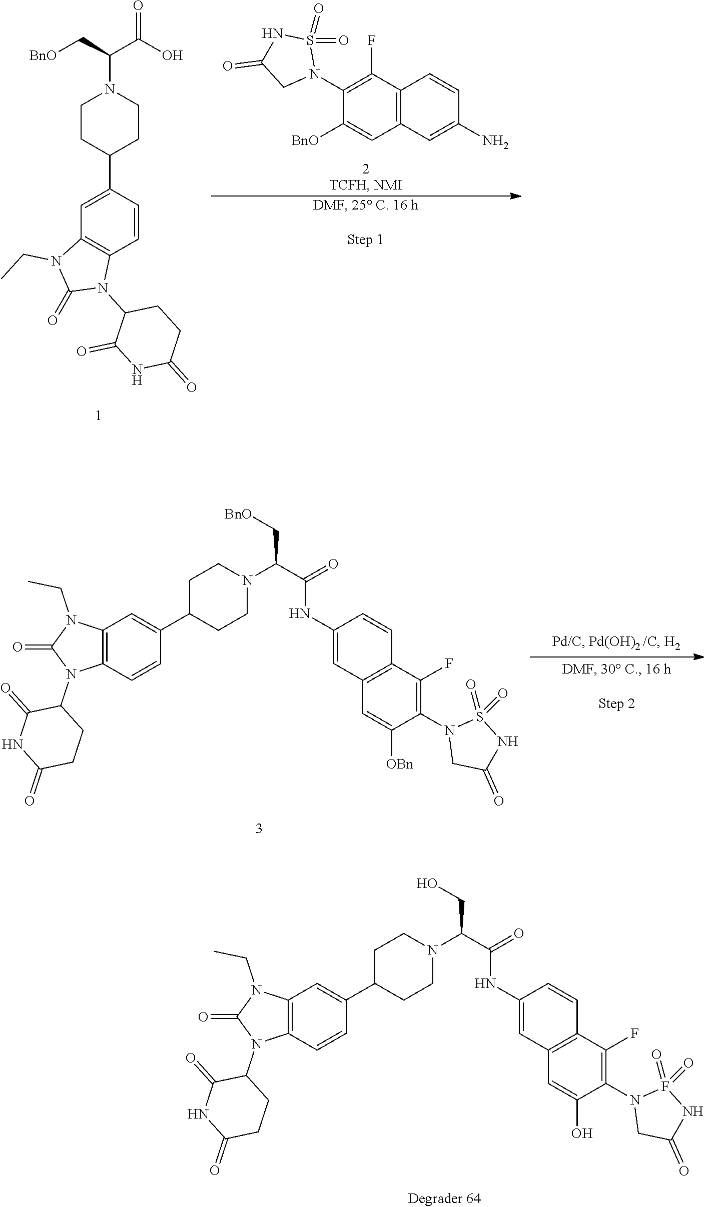

### Step 1: (2S)-3-(benzyloxy)-N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)propanamide (3)

To a mixture of (2S)-3-(benzyloxy)-2-(4-(1-(2,6-dioxopiperidin-3-yl)-3-ethyl-2-oxo-2,3-dihydro-1H-benzo[d]imidazol-5-yl)piperidin-1-yl)propanoic acid (1, 142 mg, 218.92 μmol, TFA salt, 1 eq), 5-(6-amino-3-(benzyloxy)-1-fluoronaphthalen-2-yl)-1,2,5-thiadiazolidin-3-one 1,1-dioxide (2, 112.84 mg, 218.92 μmol, TFA salt, 1 eq) and NMI (89.87 mg, 1.09 mmol, 5 eg) in DMF (10 mL) was added TCFH (153.56 mg, 547.31 μmol, 32.58 μL, 2.5 eq) at 0° C., the mixture was stirred at 20° C. for 16 h. The mixture was concentrated in vacuum. The reaction mixture was purified by prep-HPLC (flow: 25 mL/min; gradient: from 45-75% MeCN in water (0.1% TFA); column: Phenomenex luna C18 150×40 mm×15 um) to afford (2S)-3-(benzyloxy)-N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)propanamide (3, 110 mg, 104.46 μmol, 48% yield, TFA salt) as yellow solid.

LCMS (ESI): m/z 918.4 [M+H]⁺

### Step 2: (2S)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)-3-hydroxypropanamide (Degrader 64)

To a solution of (2S)-3-(benzyloxy)-N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)propanamide (3, 110 mg, 119.83 μmol, TFA salt) in DMF (5 mL) was added Pd/C (100 mg, 10% purity) and Pd(OH)₂/C (100 mg, 10% purity) under H₂ (15 Psi), the mixture was stirred at 30° C. for 16 h under H₂ (15 Psi) atmosphere. The mixture was filtered and concentrated in vacuum. The residue was purified by prep-HPLC (flow: 25 mL/min; gradient: from 55-85% MeCN in water (0.1% TFA); column: Phenomenex Synergi C18 150×25 mm×10 um) to afford (2S)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)-3-hydroxypropanamide (Degrader 64, 61.76 mg, 72.51 μmol, 61% yield, TFA salt) as white solid.

LCMS (ESI): m/z 738.2 [M+H]⁺

¹H NMR (400 MHz, DMSO-d₆) δ 11.09 (s, 1H), 10.80 (br s, 1H), 9.95 (br s, 1H), 9.68 (br dd, J=3. 6.4 Hz, 1H), 8.15 (s, 1H), 7.92 (d, J=8.8 Hz, 1H), 7.50 (dd, J=1.6, 9.0 Hz, 1H), 7.11-7.06 (m, 2H), 7.02 (s, 1H), 6.94 (br d, J=8.0 Hz, 1H), 5.88-5.73 (m, 1H), 5.35 (dd, J=5.6, 12.8 Hz, 1H), 4.16-4.11 (m, 4H), 3.89 (br d, J=7.2 Hz, 2H), 3.79-3.74 (m, 1H), 3.39-3.28 (m, 3H), 2.91 (br t, J=12.0 Hz, 2H), 2.72-2.65 (m, 2H), 2.28-1.97 (m, 6H), 1.24 (t, J=7.2 Hz, 3H).

### 2-[(3S)-4-[1-(2,6-dioxo-3-piperidyl)-6-fluoro-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 65)

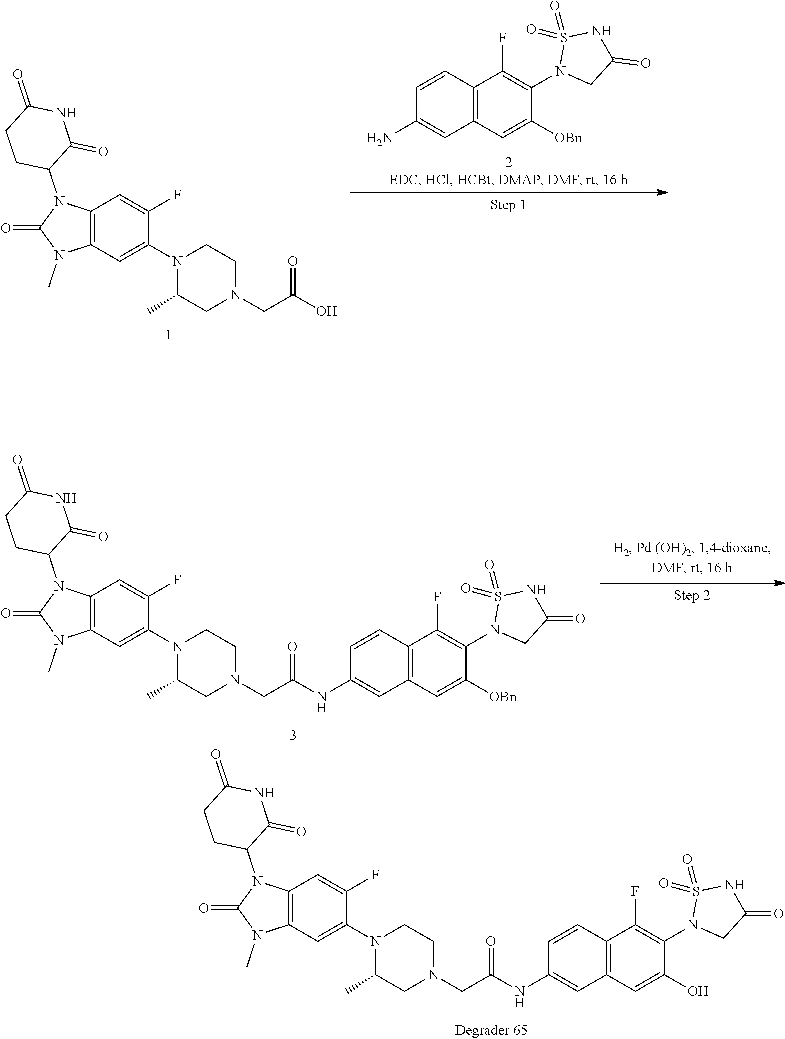 [SMILES: C[C@H]1CN(CC(=O)O)CCN1c1cc2c(cc1F)n(C1CCC(=O)NC1=O)c(=O)n2C ; Nc1ccc2c(F)c(N3CC(=O)NS3(=O)=O)c(OCc3ccccc3)cc2c1 ; C[C@H]1CN(CC(=O)Nc2ccc3c(F)c(N4CC(=O)NS4(=O)=O)c(OCc4ccccc4)cc3c2)CCN1c1cc2c(cc1F)n(C1CCC(=O)NC1=O)c(=O)n2C ; C[C@H]1CN(CC(=O)Nc2ccc3c(F)c(N4CC(=O)NS4(=O)=O)c(O)cc3c2)CCN1c1cc2c(cc1F)n(C1CCC(=O)NC1=O)c(=O)n2C]

### Step 1: N-[7-benzyloxy-5-fluoro-6-(1,1,4-trioxo-1,2,5-thiadiazolidin-2-yl)-2-naphthyl]-2-[(3S)-4-[1-(2,6-dioxo-3-piperidyl)-6-fluoro-3-methyl-2-oxo-benzimidazol-5-yl]-3-methyl-piperazin-1-yl]acetamide (3)

Into a 25 mL single neck round-bottom flask containing a solution of 2-[(3S)-4-[1-(2,6-dioxo-3-piperidyl)-6-fluoro-3-methyl-2-oxo-benzimidazol-5-yl]-3-methyl-piperazin-1-yl]acetic acid (1, 230 mg, 415.92 μmol, TFA salt) and 5-(6-amino-3-benzyloxy-1-fluoro-2-naphthyl)-1,1-dioxo-1,2,5-thiadiazolidin-3-one 2, 221.01 mg, 415.92 μmol, TFA salt) in anhydrous DMF (8 mL) were added EDC·HCl (318.93 mg, 1.66 mmol), HOBt (140.50 mg, 1.04 mmol) and DMAP (355.69 mg, 2.91 mmol). After 16 h, the solvent was removed under reduced pressure and the residue was treated with aqueous 1.5N HCl (30 mL). The precipitate was filtered, dried under reduced pressure and purified by reverse phase column chromatography [Silicycle C18 column; Mobile phase A: 0.1% Formic acid in water; Mobile phase B: MeCN] to get N-[7-benzyloxy-5-fluoro-6-(1,1,4-trioxo-1,2,5-thiadiazolidin-2-yl)-2-naphthyl]-2-[(3 S)-4-[1-(2,6-dioxo-3-piperidyl)-6-fluoro-3-methyl-2-oxo-benzimidazol-5-yl]-3-methyl-piperazin-1-yl]acetamide (3, 160 mg, 173.19 μmol, 42% yield, Formic acid salt) as white solid.

LCMS (ES+): m/z 817.0 [M+H]⁺

### Step 2: 2-[(3S)-4-[1-(2,6-dioxo-3-piperidyl)-6-fluoro-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 65)

Into a 50 mL single neck round-bottom flask containing a well-stirred solution of N-[7-benzyloxy-5-fluoro-6-(1,1,4-trioxo-1,2,5-thiadiazolidin-2-yl)-2-naphthyl]-2-[(3S)-4-[1-(2,6-dioxo-3-piperidyl)-6-fluoro-3-methyl-2-oxo-benzimidazol-5-yl]-3-methyl-piperazin-1-yl]acetamide (3, 155 mg, 167.78 μmol, Formic acid salt) in anhydrous 1,4-dioxane (7 mL) and DMF (2.5 mL) was added 20 wt. % palladium hydroxide on carbon (160 mg, 227.86 μmol, 20% purity). After 16 h, the reaction mixture was filtered and the filtrate was concentrated under reduced pressure. The crude compound was purified by reverse phase prep HPLC [Purification method: Column: XBridge C8 (150×19.1) mm, 5 micron; Mobile phase A: 0.1% Formic acid in water; Mobile phase B: MeCN] to 2-[(3S)-4-[1-(2,6-dioxo-3-piperidyl)-6-fluoro-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 65, 36 mg, 48.79 μmol, 29% yield) as an off-white solid.

LCMS (ES+): m/z 727.0 [M+H]⁺

¹H NMR (400 MHz, DMSO) δ 11.08 (s, 1H), 9.76 (s, 1H), 8.13 (s, 1H), 7.87 (d, J=9.0 Hz, 1H), 7.53-7.46 (m, 1H), 7.17 (d, J=11.3 Hz, 1H), 7.13-7.07 (m, 1H), 6.98 (s, 1H), 5.33 (dd, J=12.8, 5.4 Hz, 1H), 4.08 (s, 2H), 3.35 (s, 2H), 3.16-3.05 (m, 1H), 2.94-2.69 (m, 2H), 2.65-2.57 (m, 1H), 2.55-2.52 (m, 2H), 2.48-2.45 (m, 2H), 2.04-1.95 (m, 1H), 0.90 (s, 3H). Additional protons under solvent peak.

### 2-[(3S)-4-[1-(2,6-dioxo-3-piperidyl)-4-fluoro-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 66)

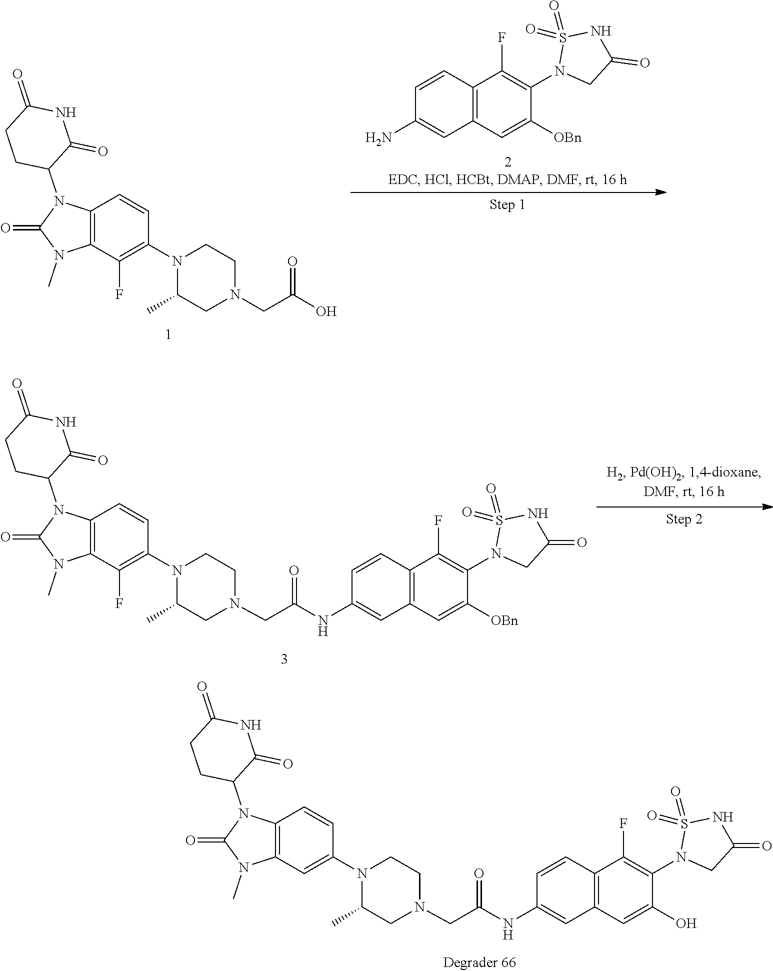 [SMILES: C[C@H]1CN(CC(=O)O)CCN1c1ccc2c(c1F)n(C)c(=O)n2C1CCC(=O)NC1=O ; Nc1ccc2c(F)c(N3CC(=O)NS3(=O)=O)c(OCc3ccccc3)cc2c1 ; C[C@H]1CN(CC(=O)Nc2ccc3c(F)c(N4CC(=O)NS4(=O)=O)c(OCc4ccccc4)cc3c2)CCN1c1ccc2c(c1F)n(C)c(=O)n2C1CCC(=O)NC1=O ; C[C@H]1CN(CC(=O)Nc2ccc3c(F)c(N4CC(=O)NS4(=O)=O)c(O)cc3c2)CCN1c1ccc2c(c1)n(C)c(=O)n2C1CCC(=O)NC1=O]

2-[(3 S)-4-[1-(2,6-dioxo-3-piperidyl)-4-fluoro-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 66, 70 mg, 86.27 μmol, Formic acid salt) was synthesized following the same two step procedure as 2-[(3S)-4-[1-(2,6-dioxo-3-piperidyl)-6-fluoro-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 65) LCMS (ES+): m/z 727.1 [M+H]⁺

¹HNMR (400 MHz, DMSO-d6): δ 11.15 (s, 1H), 10.79 (bs, 1H), 10.43 (bs, 1H), 9.85 (s, 1H), 8.14 (s, 1H), 7.89 (d, J=9.20 Hz, 1H), 7.49 (d, J=9.20 Hz, 1H), 6.99-6.97 (m, 3H), 5.41-5.36 (m, 1H), 4.19-4.39 (m, 1H), 4.09 (s, 2H), 3.65-3.51 (m, 2H), 3.50 (s, 3H), 3.14-2.94 (m, 2H), 2.91-2.86 (m, 1H), 2.74-2.62 (m, 3H), 2.05-2.02 (m, 1H), 0.90 (bs, 3H).

### 3-(5-(4-(2-(3-((7-(1,1-dioxido-4-oxo-1,2,5-thiadiazolidin-2-yl)-8-fluoro-6-hydroxynaphthalen-2-yl)oxy) azetidin-1-yl)-2-oxoethyl)-4-hydroxypiperidin-1-yl)-3-methyl-2-oxo-2,3-dihydro-1H-benzo[d]imidazol-1-yl)piperidine-2,6-dione (Degrader 67)

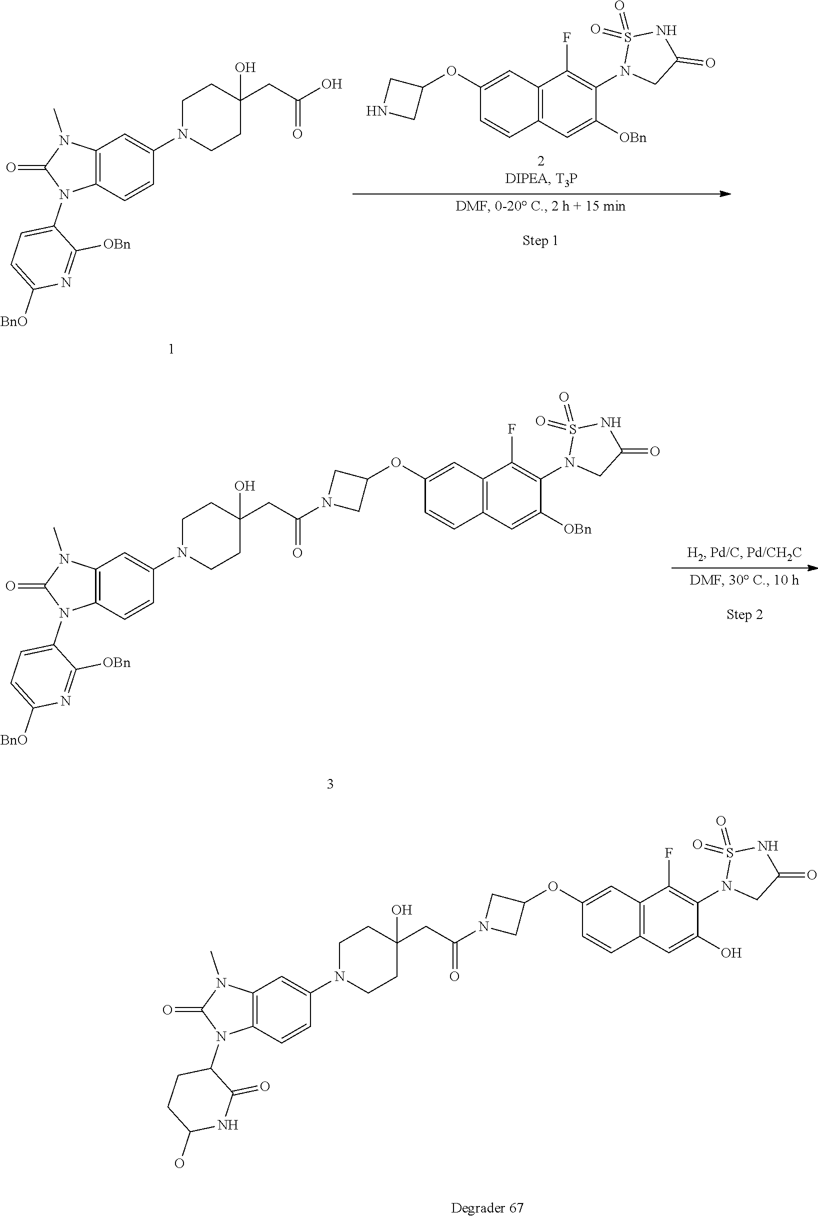 [SMILES: Cn1c(=O)n(-c2ccc(OCc3ccccc3)nc2OCc2ccccc2)c2ccc(N3CCC(O)(CC(=O)O)CC3)cc21 ; O=C1CN(c2c(OCc3ccccc3)cc3ccc(OC4CNC4)cc3c2F)S(=O)(=O)N1 ; Cn1c(=O)n(-c2ccc(OCc3ccccc3)nc2OCc2ccccc2)c2ccc(N3CCC(O)(CC(=O)N4CC(Oc5ccc6cc(OCc7ccccc7)c(N7CC(=O)NS7(=O)=O)c(F)c6c5)C4)CC3)cc21 ; Cn1c(=O)n(C2CCC(O)NC2=O)c2ccc(N3CCC(O)(CC(=O)N4CC(Oc5ccc6cc(O)c(N7CC(=O)NS7(=O)=O)c(F)c6c5)C4)CC3)cc21]

### Step 1: 5-(3-(benzyloxy)-7-((1-(2-(1-(1-(2,6-bis(benzyloxy)pyridin-3-yl)-3-methyl-2-oxo-2,3-dihydro-1H-benzo[d]imidazol-5-yl)-4-hydroxypiperidin-4-vl)acetyl)azetidin-3-yl)oxy)-1-fluoronaphthalen-2-vl)-1,2,5-thiadiazolidin-3-one 1,1-dioxide (3)

To a solution of 2-(1-(1-(2,6-bis(benzyloxy)pyridin-3-yl)-3-methyl-2-oxo-2,3-dihydro-1H-benzo[d]imidazol-5-yl)-4-hydroxypiperidin-4-yl)acetic acid (1, 150 mg, 211.66 μmol, 061) in DMF (3 mL) was added DIEA (136.78 mg, 1.06 mmol, 184.34 μL, 5 eq) and HATU (89.00 mg, 232.83 μmol, 1.1 eq) at 0° C. The mixture was stirred at 20° C. for 15 min. Then the mixture was added 5-(7-(azetidin-3-yloxy)-3-(benzyloxy)-1-fluoronaphthalen-2-yl)-1,2,5-thiadiazolidin-3-one 1,1-dioxide (2, 120.96 mg, 211.66 μmol, 1 eq). The mixture was stirred at 20° C. for 2 h under N₂. The reaction mixture was filtered and concentrated under reduced pressure. The reaction solution was purified by prep-HPLC (flow: 25 mL/min; gradient: from 60-30% water (0.05% ammonia hydroxide v/v)-ACN; column: Waters Xbridge 150×25 mm×5 um) to afford 5-(3-(benzyloxy)-7-((1-(2-(1-(1-(2,6-bis(benzyloxy)pyridin-3-yl)-3-methyl-2-oxo-2,3-dihydro-1H-benzo[d]imidazol-5-yl)-4-hydroxypiperidin-4-yl)acetyl)azetidin-3-yl)oxy)-1-fluoronaphthalen-2-yl)-1,2,5-thiadiazolidin-3-one 1,1-dioxide (3, 150 mg, 145.05 μmol, 69% yield) as a off-white solid.

LCMS (ESI): m/z 1034.3 [M+H]⁺

### Step 2: 3-(5-(4-(2-(3-((7-(1,1-dioxido-4-oxo-1,2,5-thiadiazolidin-2-yl)-8-fluoro-6-hydroxynaphthalen-2-vl)oxy)azetidin-1-yl)-2-oxoethyl)-4-hydroxypiperidin-1-vl)-3-methyl-2-oxo-2,3-dihydro-1H-benzo[d]imidazol-1-yl)piperidine-2,6-dione (Degrader 67)

To a solution of 5-(3-(benzyloxy)-7-((1-(2-(1-(1-(2,6-bis(benzyloxy)pyridin-3-yl)-3-methyl-2-oxo-2,3-dihydro-1H-benzo[d]imidazol-5-yl)-4-hydroxypiperidin-4-yl)acetyl)azetidin-3-yl)oxy)-1-fluoronaphthalen-2-yl)-1,2,5-thiadiazolidin-3-one 1,1-dioxide (3, 50 mg, 48.35 μmol) in DMF (2 mL) was added Pd/C (50 mg, 10% purity) and Pd(OH)₂/C (50 mg, 10% purity). The mixture was stirred at 30° C. for 16 h under H₂ (15 Psi). The reaction mixture was filtered and concentrated under reduced pressure to give a residue. The residue was purified by prep-HPLC (flow: 25 mL/min; gradient: from 44%-14% water (0.1% TFA)-ACN; column: 3_Phenomenex Luna C18 75×30 mm×3 um) to afford 3-(5-(4-(2-(3-((7-(1,1-dioxido-4-oxo-1,2,5-thiadiazolidin-2-yl)-8-fluoro-6-hydroxynaphthalen-2-yl)oxy)azetidin-1-yl)-2-oxoethyl)-4-hydroxypiperidin-1-yl)-3-methyl-2-oxo-2,3-dihydro-1H-benzo[d]imidazol-1-yl)piperidine-2,6-dione (Degrader 67, 27.29 mg, 30.71 μmol, 64% yield, TFA salt) as a white solid.

LCMS (ESI): m/z 766.5 [M+H]⁺

¹H NMR (400 MHz, DMSO-d6) δ 11.12 (s, 1H), 10.09-9.85 (m, 1H), 7.76 (d, J=9.0 Hz, 1H), 7.64-7.35 (m, 1H), 7.32-7.12 (m, 3H), 7.07 (s, 1H), 7.00 (d, J=2.1 Hz, 1H), 5.39 (br dd, J=5.1, 12.6 Hz, 1H), 5.24-5.17 (m, 1H), 4.78-4.71 (m, 1H), 4.39 (br dd, J=6.7, 10.8 Hz, 1H), 4.30-4.22 (m, 3H), 4.20 (br s, 1H), 3.91 (br dd, J=3.1, 10.4 Hz, 2H), 3.51-3.42 (m, 2H), 3.38 (s, 3H), 2.96-2.84 (m, 1H), 2.75-2.62 (m, 2H), 2.40 (br d, J=5.6 Hz, 2H), 2.17-1.99 (m, 3H), 1.93-1.82 (m, 2H)

### N-(6-(1,1-dioxido-4-oxo-1,2,5-thiadiazolidin-2-yl)-5-fluoro-7-hydroxynaphthalen-2-yl)-2-((1-(1-(2,6-dioxopiperidin-3-yl)-3-methyl-2-oxo-2,3-dihydro-1H-benzo[d]imidazol-5-yl)piperidin-4-yl)methyl)cyclopropanecarboxamide (Degrader 68)

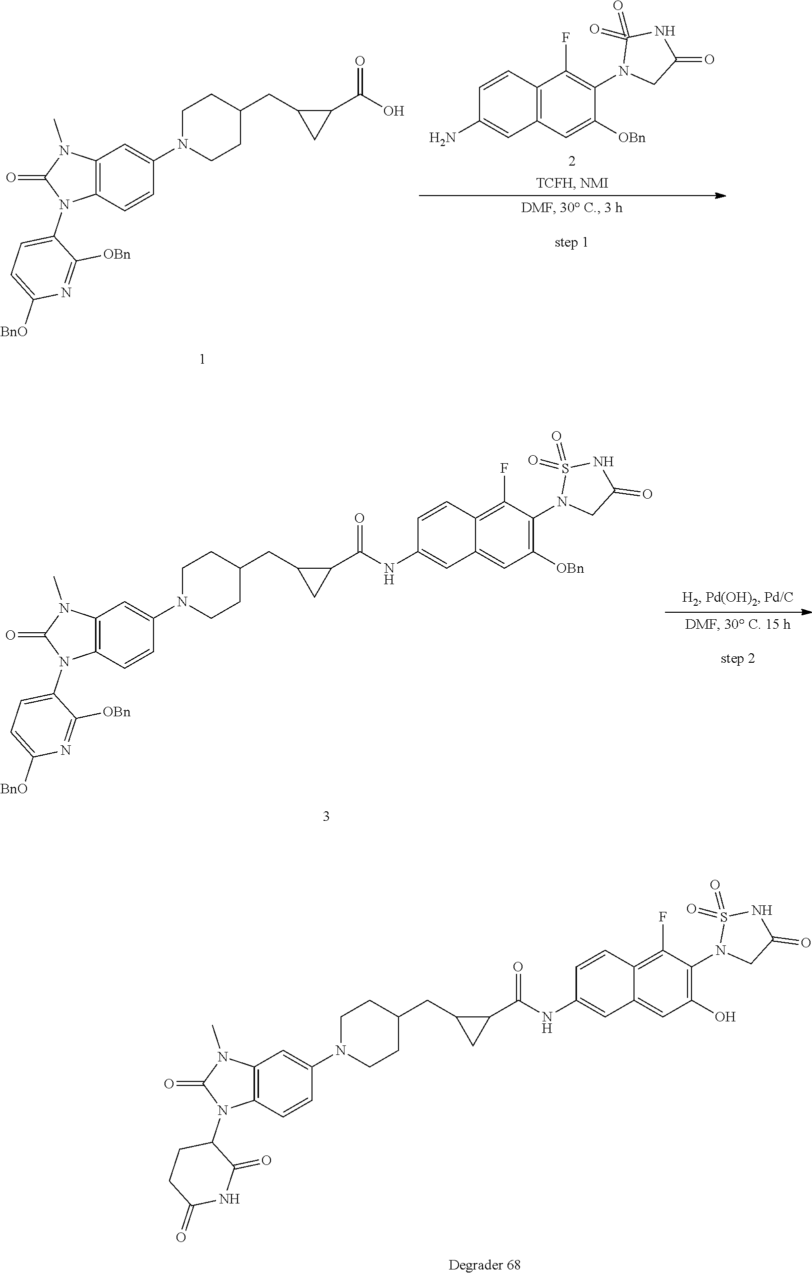

### Step 1: N-(7-(benzyloxy)-6-(1,1-dioxido-4-oxo-1,2,5-thiadiazolidin-2-yl)-5-fluoronaphthalen-2-yl)-2-((1-(1-(2,6-bis(benzyloxy)pyridin-3-yl)-3-methyl-2-oxo-2,3-dihydro-1H-benzo[d]imidazol-5-yl)piperidin-4-yl)methyl)cyclopropanecarboxamide (3)

To a solution of 2-((1-(1-(2,6-bis(benzyloxy)pyridin-3-yl)-3-methyl-2-oxo-2,3-dihydro-1H-benzo[d]imidazol-5-yl)piperidin-4-yl)methyl)cyclopropanecarboxylic acid (1, 330 mg, 533.36 μmol, 1 eq) and 5-(6-amino-3-(benzyloxy)-1-fluoronaphthalen-2-yl)-1,2,5-thiadiazolidin-3-one 1,1-dioxide (2, 288.66 mg, 560.03 μmol, TFA salt, 1.05 eq in DMF (10 mL) was added NMI (218.95 mg, 2.67 mmol, 212.58 μL, 5 eq. The reaction mixture added TCFH (374.12 mg, 1.33 mmol, 2.5 eq) at 0° C. The mixture was stirred at 30° C. for 3 h. The reaction mixture was filtered and the filtrate was concentrated under reduced pressure to give residue. The residue was purified by prep-HPLC (flow: 25 mL/min; gradient: from 30-60% MeCN-water (0.05% ammonia hydroxide v/v) over 10 min; column: Phenomenex Gemini-NX C18 75×30 mm×3 um) to afford N-(7-(benzyloxy)-6-(1,1-dioxido-4-oxo-1,2,5-thiadiazolidin-2-yl)-5-fluoronaphthalen-2-yl)-2-((1-(1-(2,6-bis(benzyloxy)pyridin-3-yl)-3-methyl-2-oxo-2,3-dihydro-1H-benzo[d]imidazol-5-yl)piperidin-4-yl)methyl)cyclopropanecarboxamide (3, 250 mg, 246.98 μmol, 46% yield) as a white solid.

LCMS (ESI): m/z 1002.3 [M+H]⁺

¹H NMR (400 MHz, d₆-DMSO) δ 10.45 (s, 1H), 8.28 (s, 1H), 7.89 (d, J=8.8 Hz, 1H), 7.80 (d, J=8.0 Hz, 1H), 7.60-7.50 (m, 3H), 7.48-7.42 (m, 2H), 7.42-7.30 (m, 6H), 7.29-7.26 (m, 4H), 7.21 (d, J=4.4 Hz, 1H), 7.09 (s, 1H), 7.01-6.92 (m, 1H), 6.79-6.54 (m, 2H), 5.46-5.32 (m, 4H), 5.27 (s, 2H), 4.16 (s, 2H), 3.61 (d, J=9.2 Hz, 3H), 3.38 (s, 3H), 1.94 (d, J=10.0 Hz, 2H), 1.82-1.17 (m, 8H), 1.14-1.03 (m, 1H), 0.82-0.70 (m, 1H).

### Step 2: N-(6-(1,1-dioxido-4-oxo-1,2,5-thiadiazolidin-2-yl)-5-fluoro-7-hydroxynaphthalen-2-yl)-2-((1-(1-(2,6-dioxopiperidin-3-yl)-3-methyl-2-oxo-2,3-dihydro-1H-benzo[d]imidazol-5-yl)piperidin-4-yl)methyl)cyclopropanecarboxamide (Degrader 68)

To a solution of N-(7-(benzyloxy)-6-(1,1-dioxido-4-oxo-1,2,5-thiadiazolidin-2-yl)-5-fluoronaphthalen-2-yl)-2-((1-(1-(2,6-bis(benzyloxy)pyridin-3-yl)-3-methyl-2-oxo-2,3-dihydro-1H-benzo[d]imidazol-5-yl)piperidin-4-yl)methyl)cyclopropanecarboxamide (3, 50 mg, 49.89 μmol, 1 eq) in DMF (5 mL) were added Pd/C (50 mg, 10% purity) and Pd(OH)₂/C (50 mg, 10% purity) under N₂ atmosphere. The suspension was degassed and purged with H₂ 3 times. The mixture was stirred under H₂ (15 psi) at 30° C. for 16 h. The reaction mixture was filtered and the filtrate was concentrated under reduced pressure to give residue. The residue was purified by prep-HPLC (flow: 25 mL/min; gradient: from 17-47% water (0.1% TFA)-ACN over 9 min; column: Phenomenex Synergi C18 150×25 mm×10 um) to afford N-(6-(1,1-dioxido-4-oxo-1,2,5-thiadiazolidin-2-yl)-5-fluoro-7-hydroxynaphthalen-2-yl)-2-((1-(1-(2,6-dioxopiperidin-3-yl)-3-methyl-2-oxo-2,3-dihydro-1H-benzo[d]imidazol-5-yl)piperidin-4-yl)methyl)cyclopropanecarboxamide (Degrader 68, 31.41 mg, 35.94 μmol, 72% yield) as a white solid.

LCMS (ESI): m/z 734.2 [M+H]⁺

¹H NMR (400 MHz, d₆-DMSO) δ 11.12 (s, 1H), 10.41 (s, 1H), 10.11-9.84 (m, 1H), 8.16 (s, 1H), 7.84 (d, J=8.8 Hz, 1H), 7.45 (dd, J=1.6, 8.8 Hz, 2H), 7.27-6.97 (m, 2H), 6.94 (s, 1H), 5.48-5.23 (m, 1H), 4.22 (s, 2H), 3.63 (s, 3H), 3.36 (s, 3H), 2.94-2.86 (m, 1H), 2.76-2.68 (m, 1H), 2.65 (s, 1H), 2.07-1.90 (m, 4H), 1.82-1.74 (m, 1H), 1.70-1.57 (m, 3H), 1.43-1.32 (m, 3H), 1.12-1.06 (m, 1H), 0.80-0.74 (m, 1H).

### N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-3-methoxyphenyl)acetamide (Degrader 69)

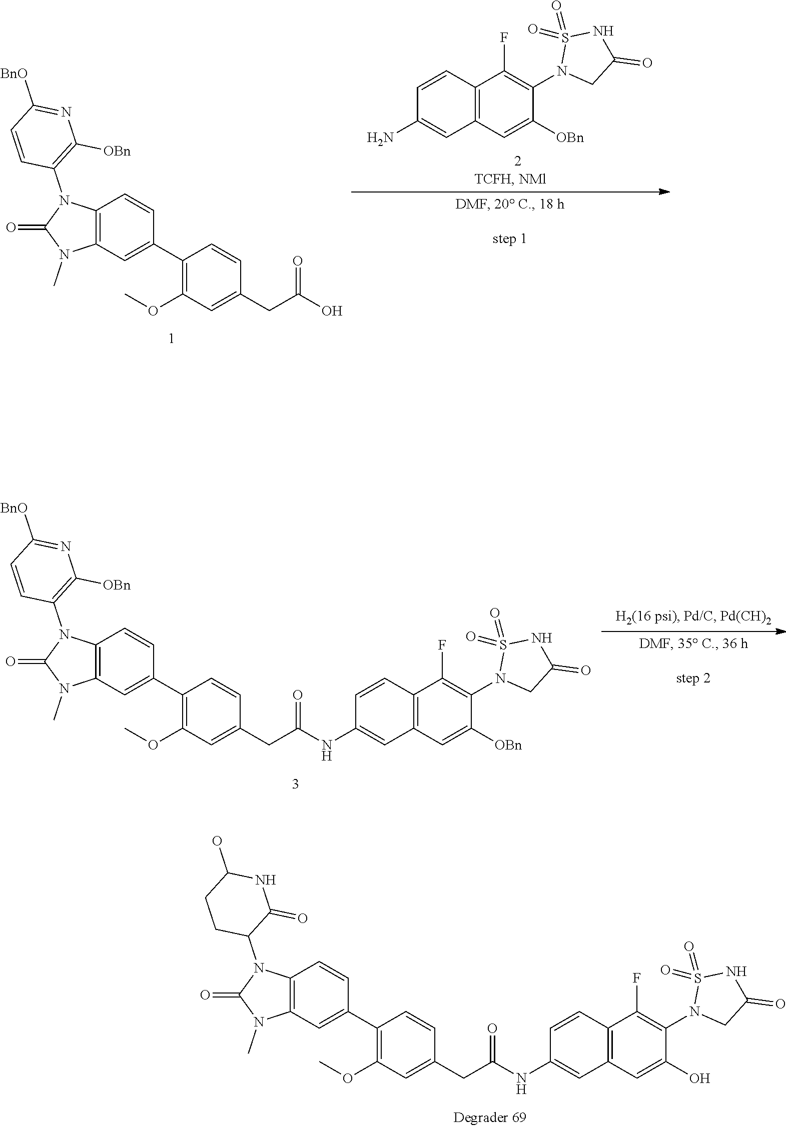 [SMILES: COc1cc(CC(=O)O)ccc1-c1ccc2c(c1)n(C)c(=O)n2-c1ccc(OCc2ccccc2)nc1OCc1ccccc1 ; Nc1ccc2c(F)c(N3CC(=O)NS3(=O)=O)c(OCc3ccccc3)cc2c1 ; COc1cc(CC(=O)Nc2ccc3c(F)c(N4CC(=O)NS4(=O)=O)c(OCc4ccccc4)cc3c2)ccc1-c1ccc2c(c1)n(C)c(=O)n2-c1ccc(OCc2ccccc2)nc1OCc1ccccc1 ; COc1cc(CC(=O)Nc2ccc3c(F)c(N4CC(=O)NS4(=O)=O)c(O)cc3c2)ccc1-c1ccc2c(c1)n(C)c(=O)n2C1CCC(O)NC1=O]

### Step 1: N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-3-methoxyphenyl)acetamide (3)

To a solution of 2-[4-[1-(2,6-dibenzyloxy-3-pyridyl)-3-methyl-2-oxo-benzimidazol-5-yl]-3-methoxy-phenyl]acetic acid (1, 90 mg, 149.59 μmol) and 5-(6-amino-3-(benzyloxy)-1-fluoronaphthalen-2-yl)-1,2,5-thiadiazolidin-3-one 1,1-dioxide (2, 66.05 mg, 164.55 μmol) in DMF (1 mL) was added [chloro(dimethylamino)methylene]-dimethyl-ammonium; hexafluorophosphate (104.93 mg, 373.97 μmol) and 1-methylimidazole (61.41 mg, 747.95 μmol, 59.62 uL). The reaction mixture was stirred at 20° C. for 16 h. The reaction mixture was purified by reversed phased HPLC (0.01%, NH₃·H₂O in water, MeCN). The desired peak was concentrated and lyphilizated to afford N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-3-methoxyphenyl)acetamide (3, 65 mg, 61.37 μmol, 41% yield) as white solid.

LCMS (ESI): m/z 985.2 [M+H]⁺

### Step 2: N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-3-methoxyphenyl)acetamide (Degrader 69)

To a solution of N-[7-benzyloxy-5-fluoro-6-(1,1,4-trioxo-1,2,5-thiadiazolidin-2-yl)-2-naphthyl]-2-[4-[1-(2,6-dibenzyloxy-3-pyridyl)-3-methyl-2-oxo-benzimidazol-5-yl]-3-methoxy-phenyl]acetamide (3, 65 mg, 65.99 μmol) in DMF (2 mL) was added wet Pd/C (30 mg, 10% purity) and wet dihydroxypalladium (75.00 mg, 20% purity). The suspension was degassed and purged with H₂ 3 times. The mixture was stirred under H₂ (15 psi) at 35° C. for 16 h. The reaction mixture was filtered and the filtrate was purified by prep-HPLC(Column: Phenomenex Gemini-NX C18 75*30 mm*3 um, Condition: water (0.1% TFA)-ACN, Begin B 25, End B 55, Gradient Time (7 min), FlowRate (25 ml/min)), the target peak was concentrated and lyophilizated to afford N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-3-methoxyphenyl)acetamide (Degrader 69, 28.42 mg, 33.93 μmol, 51% yield) as white solid.

LCMS (ESI): m/z 717.4 [M+H]⁺

¹H NMR (400 MHz, DMSO-d6) δ=11.09 (s, 1H), 10.20 (s, 1H), 10.13-9.97 (m, 1H), 8.14 (s, 1H), 7.82 (d, J=8.8 Hz, 1H), 7.46-7.32 (m, 1H), 7.22 (d, J=8.4 Hz, 1H), 7.16-7.09 (m, 2H), 7.04-6.99 (m, 2H), 6.96-6.89 (m, 2H), 5.39-5.35 (m, 1H), 4.24 (s, 2H), 3.80 (s, 3H), 3.68 (s, 2H), 3.22 (s, 3H), 2.94-2.84 (m, 1H), 2.61 (d, J=17.6 Hz, 2H), 2.02-1.94 (m, 1H).

### N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-3,5-dimethyl-1H-pyrazol-1-yl)acetamide (Degrader 70)

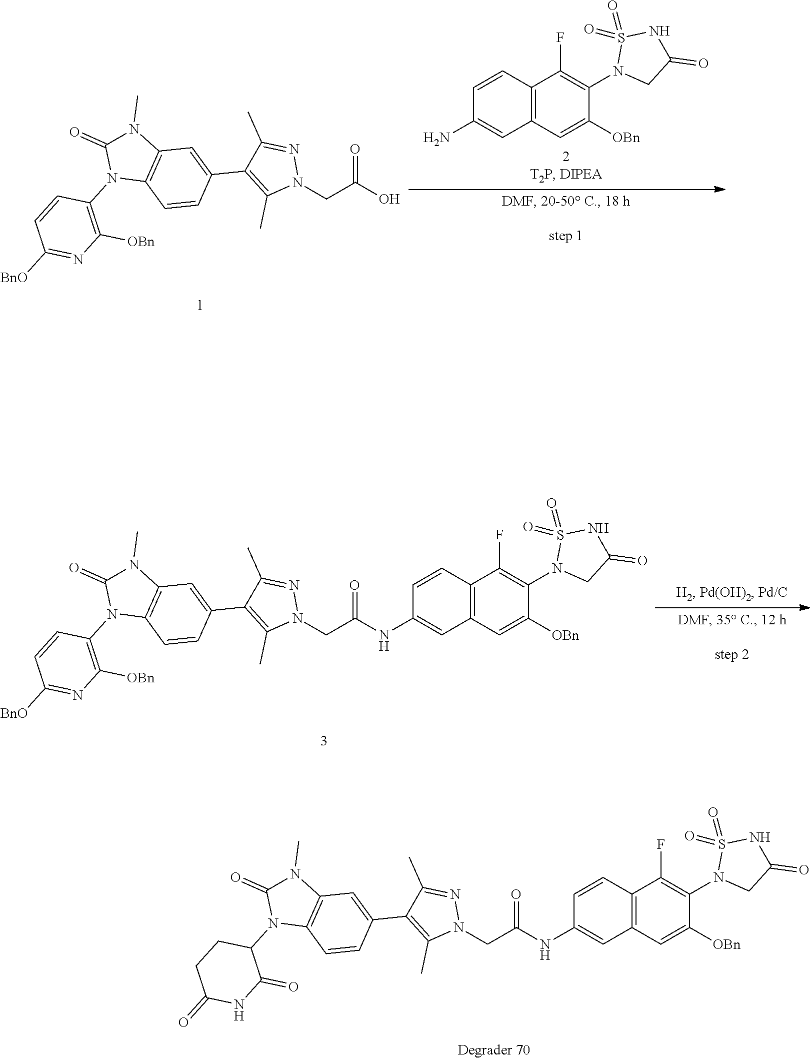 [SMILES: Cc1nn(CC(=O)O)c(C)c1-c1ccc2c(c1)n(C)c(=O)n2-c1ccc(OCc2ccccc2)nc1OCc1ccccc1 ; Nc1ccc2c(F)c(N3CC(=O)NS3(=O)=O)c(OCc3ccccc3)cc2c1 ; Cc1nn(CC(=O)Nc2ccc3c(F)c(N4CC(=O)NS4(=O)=O)c(OCc4ccccc4)cc3c2)c(C)c1-c1ccc2c(c1)n(C)c(=O)n2-c1ccc(OCc2ccccc2)nc1OCc1ccccc1 ; Cc1nn(CC(=O)Nc2ccc3c(F)c(N4CC(=O)NS4(=O)=O)c(OCc4ccccc4)cc3c2)c(C)c1-c1ccc2c(c1)n(C)c(=O)n2C1CCC(=O)NC1=O]

### Step 1: N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-3,5-dimethyl-1H-pyrazol-1-yl)acetamide (3)

To a solution of 2-(4-(1-(2,6-bis(benzyloxy)pyridin-3-yl)-3-methyl-2-oxo-2,3-dihydro-1H-benzo[d]imidazol-5-yl)-3,5-dimethyl-1H-pyrazol-1-yl)acetic acid (1, 200 mg, 339.19 μmol, 1 eq), 5-(6-amino-3-(benzyloxy)-1-fluoronaphthalen-2-yl)-1,2,5-thiadiazolidin-3-one 1,1-dioxide (2, 174.83 mg, 339.19 μmol, TFA salt, 1 eq) and DIPEA (657.57 mg, 5.09 mmol, 15 eq) in DMF (2 mL) was added T₃P (1.51 g, 2.37 mmol, 50% purity in ethyl acetate, 7 eq) at 20° C. The mixture was stirred at 50° C. for 16 h. The mixture was purified by reversed phase (NH₃·H₂O) and lyophilization to afford N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-3,5-dimethyl-1H-pyrazol-1-yl)acetamide (3, 80 mg, 82.22 μmol, 24% yield) as a white solid.

LCMS (ESI): m/z 973.6[M+H]⁺

### Step 2: N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-3,5-dimethyl-1H-pyrazol-1-yl)acetamide (Degrader 70)

To a solution of N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-3,5-dimethyl-1H-pyrazol-1-yl)acetamide (3, 70 mg, 71.94 μmol, 1 eq) in DMF (3 mL) were added Pd/C (70 mg, 10% purity) and Pd(OH)₂/C (70 mg, 20% purity) under N₂ atmosphere. The mixture was stirred under H₂ (15 Psi) at 35° C. for 12 h. The mixture was filtered and concentrated under vacuum. The residue was purified by prep-HPLC (flow: 25 mL/min; gradient: from 23-53% MeCN in water (0.1% TFA) over 7 min; column: 3_Phenomenex luna C18 75×30 mm×3 um) to afford N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-3,5-dimethyl-1H-pyrazol-1-yl)acetamide (Degrader 70, 53.2 mg, 64.98 μmol, 90% yield, TFA salt) as a pink solid.

LCMS (ESI): m/z 705.2 [M+H]⁺

1H NMR (400 MHz, DMSO-d6) δ=11.12 (s, 1H), 10.61 (s, 1H), 10.51-10.33 (m, 1H), 8.20 (s, 1H), 7.90 (d, J=8.8 Hz, 1H), 7.45 (dd, J=1.6, 9.2 Hz, 1H), 7.17 (d, J=8.0 Hz, 1H), 7.09 (d, J=0.8 Hz, 1H), 6.99-6.92 (m, 2H), 5.40 (dd, J=5.6, 12.8 Hz, 1H), 5.01 (s, 2H), 4.39 (s, 2H), 3.38 (s, 3H), 3.02-2.86 (m, 1H), 2.81-2.59 (m, 3H), 2.27 (s, 3H), 2.16 (s, 3H), 2.11-1.98 (m, 1H)

### N-(6-(1,1-dioxido-4-oxo-1,2,5-thiadiazolidin-2-yl)-5-fluoro-7-hydroxynaphthalen-2-yl)-2-(4-((1-(2,6-dioxopiperidin-3-yl)-3-methyl-2-oxo-2,3-dihydro-1H-benzo[d]imidazol-5-yl)amino)-3-fluorophenyl)acetamide (Degrader 71)

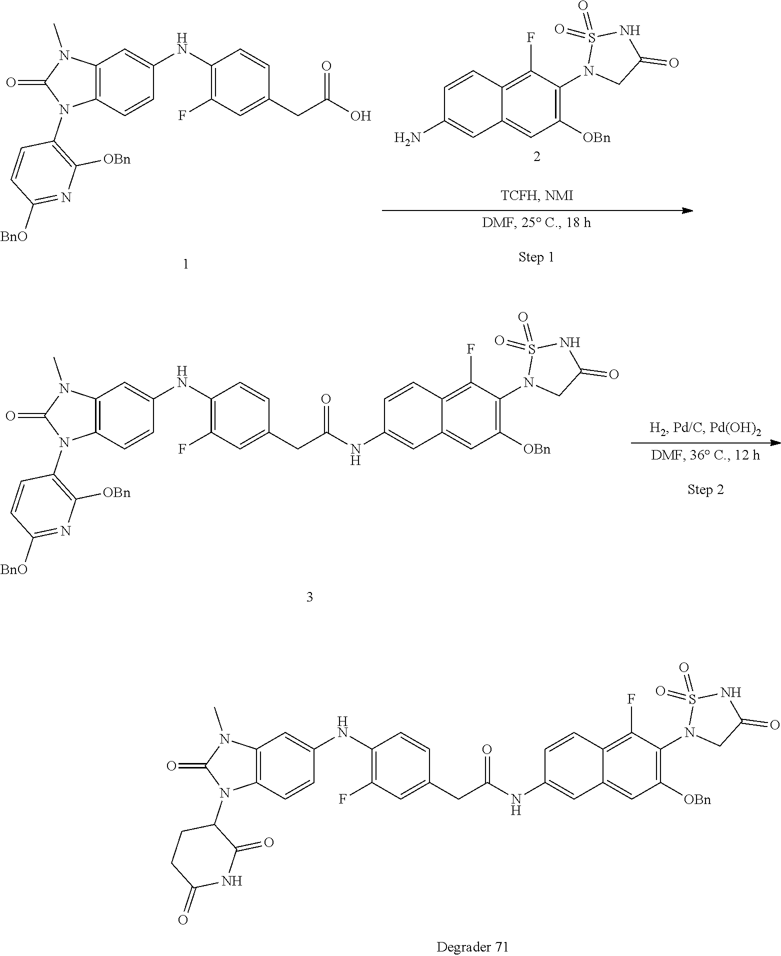 [SMILES: Cn1c(=O)n(-c2ccc(OCc3ccccc3)nc2OCc2ccccc2)c2ccc(Nc3ccc(CC(=O)O)cc3F)cc21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(Nc3ccc(CC(=O)Nc4ccc5c(F)c(N6CC(=O)NS6(=O)=O)c(OCc6ccccc6)cc5c4)cc3F)cc21 ; Cn1c(=O)n(C2CCC(=O)NC2=O)c2ccc(Nc3ccc(CC(=O)Nc4ccc5c(F)c(N6CC(=O)NS6(=O)=O)c(OCc6ccccc6)cc5c4)cc3F)cc21]

### Step 1: N-(7-(benzyloxy)-6-(1,1-dioxido-4-oxo-1,2,5-thiadiazolidin-2-yl)-5-fluoronaphthalen-2-yl)-2-(4-((1-(2,6-bis(benzyloxy)pyridin-3-yl)-3-methyl-2-oxo-2,3-dihydro-1H-benzo[d]imidazol-5-yl)amino)-3-fluorophenyl)acetamide (3)

To a solution of 2-[4-[[1-(2,6-dibenzyloxy-3-pyridyl)-3-methyl-2-oxo-benzimidazol-5-yl]amino]-3-fluoro-phenyl]acetic acid (1, 180 mg, 297.70 μmol) and 5-(6-amino-3-benzyloxy-1-fluoro-2-naphthyl)-1,1-dioxo-1,2,5-thiadiazolidin-3-one (2, 153.45 mg, 297.70 μmol, 061) in DMF (2 mL) were added [chloro(dimethylamino)methylene]-dimethyl-ammonium; hexafluorophosphate (250.59 mg, 893.11 μmol) and 1-methylimidazole (122.21 mg, 1.49 mmol, 118.65 uL). The mixture was stirred at 25° C. for 12 h. The reaction mixture was filtered to get the filtrate. The residue was purified by prep-HPLC (column: Waters Xbridge 150*25 mm*5 um; mobile phase: water (0.05% ammonia hydroxide v/v)-ACN; B %: 32%-62%, 10 min). After Prep-HPLC purification, the eluent was concentrated to remove organic solvents. The residual aqueous solution was lyophilized to give N-[7-benzyloxy-5-fluoro-6-(1,1,4-trioxo-1,2,5-thiadiazolidin-2-yl)-2-naphthyl]-2-[4-[[1-(2,6-dibenzyloxy-3-pyridyl)-3-methyl-2-oxo-benzimidazol-5-yl]amino]-3-fluoro-phenyl]acetamide (3, 65 mg, 63.81 μmol, 21% yield) as a yellow solid.

LCMS (ES+): m/z 988.5 [M+H]+

### Step 2: 2-[4-[[1-(2,6-dioxo-3-piperidyl)-3-methyl-2-oxo-benzimidazol-5-yl]amino]-3-fluoro-phenyl]-N-[5-fluoro-7-hydroxy-6-(1,1,4-trioxo-1,2,5-thiadiazolidin-2-yl)-2-naphthyl]acetamide (Degrader 71)

To a solution of N-[7-benzyloxy-5-fluoro-6-(1,1,4-trioxo-1,2,5-thiadiazolidin-2-yl)-2-naphthyl]-2-[4-[[1-(2,6-dibenzyloxy-3-pyridyl)-3-methyl-2-oxo-benzimidazol-5-yl]amino]-3-fluoro-phenyl]acetamide (3, 50 mg, 50.61 μmol) in DMF (3 mL) was added Pd/C (50 mg, 10% purity) and Pd(OH)₂/C (50 mg, 20% purity) under N₂ atmosphere. The suspension was degassed and purged with H₂ for 3 times. The mixture was stirred under H₂ (15 Psi) at 35° C. for 12 h. The reaction mixture was filtered to get the filtrate. The filtrate was purified by prep-HPLC (TFA condition: column: 3_Phenomenex Luna C18 75*30 mm*3 um; mobile phase: [water (0.225% TFA)-ACN]; B %: 30%-60%, 7 min)). After Prep-HPLC purification, the eluent was concentrated to remove organic solvents. The residual aqueous solution was lyophilized to give 2-[4-[[1-(2,6-dioxo-3-piperidyl)-3-methyl-2-oxo-benzimidazol-5-yl]amino]-3-fluoro-phenyl]-N-[5-fluoro-7-hydroxy-6-(1,1,4-trioxo-1,2,5-thiadiazolidin-2-yl)-2-naphthyl]acetamide (Degrader 71, 29.22 mg, 39.95 μmol, 79% yield) as yellow gum.

LCMS (ES+): m/z 720.2 [M+H]⁺

¹H NMR (400 MHz, DMSO-d₆) δ=11.08 (s, 1H), 10.36 (s, 2H), 8.17 (s, 1H), 7.86 (d, J=9.2 Hz, 1H), 7.46-7.44 (m, 1H), 7.24-7.12 (m, 3H), 7.06-6.93 (m, 4H), 6.89 (d, J=1.6 Hz, 1H), 6.78-6.72 (m, 1H), 5.33-5.31 (m, 1H), 4.28 (s, 2H), 3.63 (s, 2H), 3.28 (s, 3H), 2.93-2.88 (m, 1H), 2.69-2.67 (m, 2H), 2.05-2.00 (m, 1H)

### N-(6-(1,1-dioxido-4-oxo-1,2,5-thiadiazolidin-2-yl)-5-fluoro-7-hydroxynaphthalen-2-yl)-2-(3-((1-(2,6-dioxopiperidin-3-yl)-3-methyl-2-oxo-2,3-dihydro-1H-benzo[d]imidazol-5-yl)amino)phenyl)acetamide (Degrader 72)

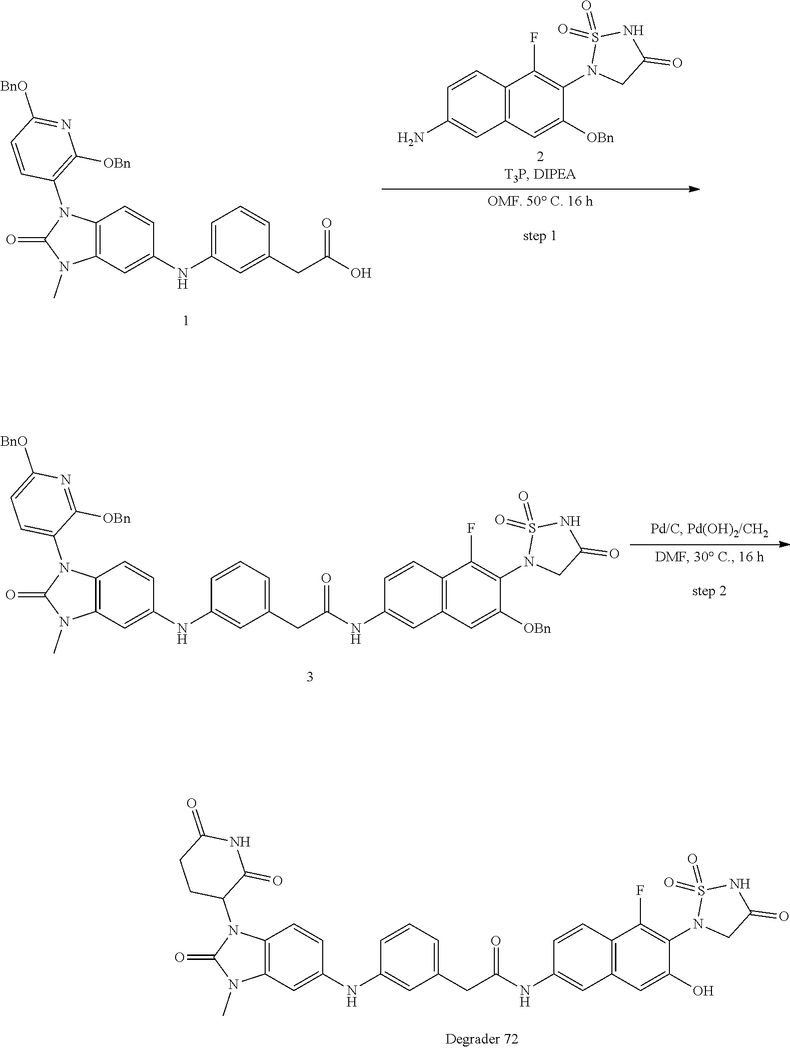 [SMILES: Cn1c(=O)n(-c2ccc(OCc3ccccc3)nc2OCc2ccccc2)c2ccc(Nc3cccc(CC(=O)O)c3)cc21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(Nc3cccc(CC(=O)Nc4ccc5c(F)c(N6CC(=O)NS6(=O)=O)c(OCc6ccccc6)cc5c4)c3)cc21 ; Cn1c(=O)n(C2CCC(=O)NC2=O)c2ccc(Nc3cccc(CC(=O)Nc4ccc5c(F)c(N6CC(=O)NS6(=O)=O)c(O)cc5c4)c3)cc21]

### Step 1: N-(7-(benzyloxy)-6-(1,1-dioxido-4-oxo-1,2,5-thiadiazolidin-2-yl)-5-fluoronaphthalen-2-yl)-2-(3-((1-(2,6-bis(benzyloxy)pyridin-3-yl)-3-methyl-2-oxo-2,3-dihydro-1H-benzo[d]imidazol-5-yl)amino)phenyl)acetamide (3)

To a mixture of 2-[3-[[1-(2,6-dibenzyloxy-3-pyridyl)-3-methyl-2-oxo-benzimidazol-5-yl]amino]phenyl]acetic acid (1, 130 mg, 221.60 μmol, 1 eq), 5-(6-amino-3-(benzyloxy)-1-fluoronaphthalen-2-yl)-1,2,5-thiadiazolidin-3-one 1,1-dioxide (2, 88.95 mg, 221.60 μmol, 1 eq) and DIPEA (429.61 mg, 3.32 mmol, 578.99 μL, 15 eq) in DMF (2 mL) was added T₃P (987.14 mg, 1.55 mmol, 50% purity in EtOAc, 7 eq), the mixture was stirred at 50° C. for 4 h. The mixture was poured into water (20 mL). The mixture was extracted with ethyl acetate (20 mL*2). The organic phase was washed with brine (30 mL), dried with anhydrous Na₂SO₄, filtered and concentrated in vacuum. The residue was purified by prep-HPLC (flow: 25 mL/min; gradient: from 30-60%; column: Waters Xbridge 150*25 mm*5 um, water (0.05% ammonia hydroxide v/v)-ACN) to afford N-(7-(benzyloxy)-6-(1,1-dioxido-4-oxo-1,2,5-thiadiazolidin-2-yl)-5-fluoronaphthalen-2-yl)-2-(3-((1-(2,6-bis(benzyloxy)pyridin-3-yl)-3-methyl-2-oxo-2,3-dihydro-1H-benzo[d]imidazol-5-yl)amino)phenyl)acetamide (3, 65 mg, 67.01 μmol, 30% yield) as yellow solid.

LCMS (ESI): m/z 970.2 [M+H]+

### Step 2: N-(6-(1,1-dioxido-4-oxo-1,2,5-thiadiazolidin-2-yl)-5-fluoro-7-hydroxynaphthalen-2-yl)-2-(3-((1-(2,6-dioxopiperidin-3-yl)-3-methyl-2-oxo-2,3-dihydro-1H-benzo[d]imidazol-5-yl)amino)phenyl)acetamide (Degrader 72)

To a mixture of N-(7-(benzyloxy)-6-(1,1-dioxido-4-oxo-1,2,5-thiadiazolidin-2-yl)-5-fluoronaphthalen-2-yl)-2-(3-((1-(2,6-bis(benzyloxy)pyridin-3-yl)-3-methyl-2-oxo-2,3-dihydro-1H-benzo[d]imidazol-5-yl)amino)phenyl)acetamide (3, 58.20 mg, 60 μmol, 1 eq) in DMF (2 mL) were added Pd/C (60 mg, 10% purity) and Pd(OH)₂/C (60 mg, 20% purity) under H₂, then the mixture was stirred at 30° C. for 16 h under H₂ (15 psi) atmosphere. The mixture was filtered and filtrate was concentrated in vacuum. The residue was purified by prep-HPLC (flow: 25 mL/min; gradient: from 32-62% water (0.1% TFA)-ACN; Phenomenex Synergi C18 150*25 mm*10 um). to afford N-(6-(1,1-dioxido-4-oxo-1,2,5-thiadiazolidin-2-yl)-5-fluoro-7-hydroxynaphthalen-2-yl)-2-(3-((1-(2,6-dioxopiperidin-3-yl)-3-methyl-2-oxo-2,3-dihydro-1H-benzo[d]imidazol-5-yl)amino)phenyl)acetamide (Degrader 72, 21.08 mg, 25.84 μmol, 43% yield, TFA salt) as an white solid.

LCMS (ESI): m/z 702.3 [M+H]+

¹H NMR (400 MHz, DMSO-d₆) δ=11.08 (s, 1H), 10.35 (s, 1H), 9.97 (br s, 1H), 8.15 (s, 1H), 8.03 (br s, 1H), 7.83 (d, J=9.0 Hz, 1H), 7.42 (dd, J=1.9, 9.0 Hz, 1H), 7.16-7.11 (m, 1H), 7.06 (s, 1H), 7.00 (d, J=8.4 Hz, 1H), 6.96-6.91 (m, 2H), 6.87 (dd, J=1.4, 8.2 Hz, 1H), 6.79-6.72 (m, 2H), 5.31 (dd, J=5.4, 12.8 Hz, 1H), 4.18 (s, 2H), 3.59 (s, 2H), 3.28 (s, 3H), 2.89 (s, 1H), 2.76-2.62 (m, 2H), 2.06-1.97 (m, 1H).

### 1-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)phenyl)urea (Degrader 73)

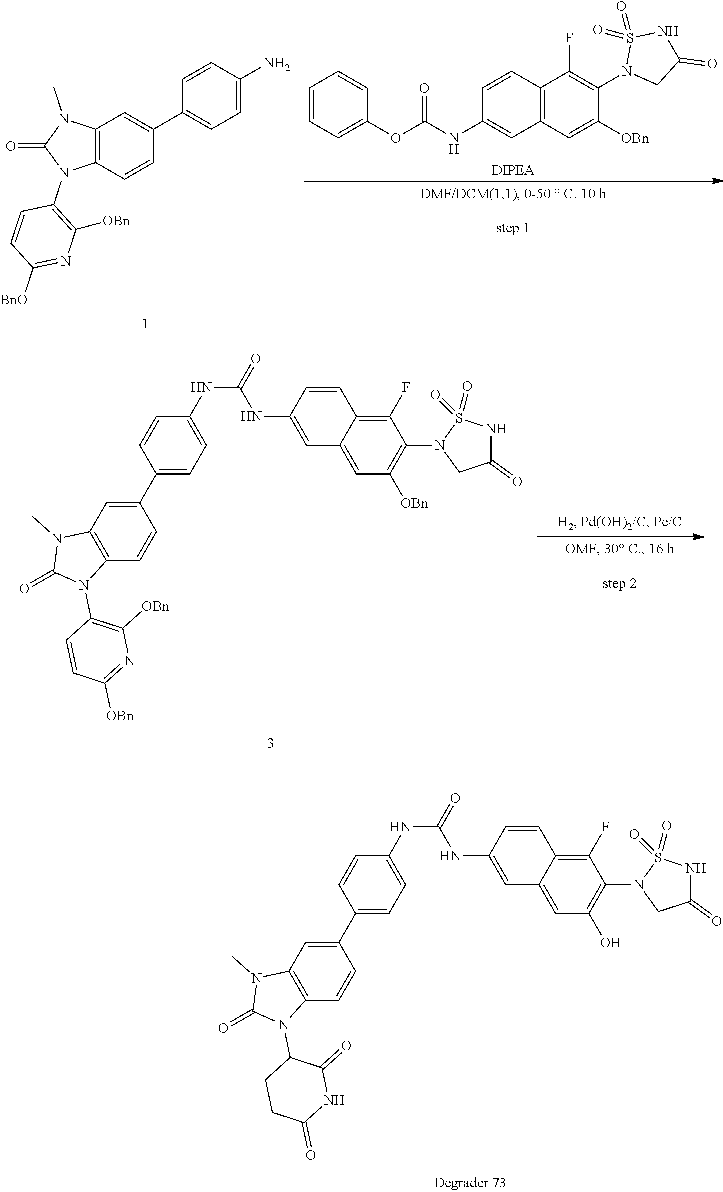 [SMILES: Cn1c(=O)n(-c2ccc(OCc3ccccc3)nc2OCc2ccccc2)c2ccc(-c3ccc(N)cc3)cc21 ; O=C1CN(c2c(OCc3ccccc3)cc3cc(NC(=O)Oc4ccccc4)ccc3c2F)S(=O)(=O)N1 ; Cn1c(=O)n(-c2ccc(OCc3ccccc3)nc2OCc2ccccc2)c2ccc(-c3ccc(NC(=O)Nc4ccc5c(F)c(N6CC(=O)NS6(=O)=O)c(OCc6ccccc6)cc5c4)cc3)cc21 ; Cn1c(=O)n(C2CCC(=O)NC2=O)c2ccc(-c3ccc(NC(=O)Nc4ccc5c(F)c(N6CC(=O)NS6(=O)=O)c(O)cc5c4)cc3)cc21]

### Step 1: 1-(7-(benzyloxy)-6-(1,1-dioxido-4-oxo-1,2,5-thiadiazolidin-2-yl)-5-fluoronaphthalen-2-yl)-3-(4-(1-(2,6-bis(benzyloxy)pyridin-3-yl)-3-methyl-2-oxo-2,3-dihydro-1H-benzo[d]imidazol-5-yl)phenyl)urea (3)

To a 5-(4-aminophenyl)-1-(2,6-bis(benzyloxy)pyridin-3-yl)-3-methyl-1H-benzo[d]imidazol-2 (3H)-one (1, 150 mg, 283.77 μmol) and N-ethyl-N-isopropyl-propan-2-amine (183.38 mg, 1.42 mmol, 247.14 μL) in DMF (4 mL) and DCM (4 mL) was added phenyl (7-(benzyloxy)-6-(1,1-dioxido-4-oxo-1,2,5-thiadiazolidin-2-yl)-5-fluoronaphthalen-2-yl)carbamate (2, 295.98 mg, 567.54 μmol) at 0′° C. The mixture was stirred at 50° C. for 16 h. The reaction mixture was filtered and the filtrate was concentrated under reduced pressure to give residue. The residue was purified by prep-HPLC (flow: 25 mL/min; gradient: from 30-60% MeCN-water (0.05% ammonia hydroxide v/v) over 10 min; column: Waters Xbridge 150×25 mm×5 um) to afford 1-(7-(benzyloxy)-6-(1,1-dioxido-4-oxo-1,2,5-thiadiazolidin-2-yl)-5-fluoronaphthalen-2-yl)-3-(4-(1 (2,6-bis(benzyloxy)pyridin-3-yl)-3-methyl-2-oxo-2,3-dihydro-1H-benzo[d]imidazol-5-yl) phenyl)urea (3,180 mg, 154.60 μmol, 55% yield) as a yellow solid.

LCMS (ESI): m/z 956.4 [M+H]+

### Step 2: 1-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)phenyl)urea (Degrader 73)

To a solution of 1-(7-(benzyloxy)-6-(1,1-dioxido-4-oxo-1,2,5-thiadiazolidin-2-yl)-5-fluoronaphthalen-2-yl)-3-(4-(1-(2,6-bis(benzyloxy)pyridin-3-yl)-3-methyl-2-oxo-2,3-dihydro-1H-benzo[d]imidazol-5-yl)phenyl)urea (3, 60 mg, 62.76 μmol) in DMF (3 mL) were added Pd(OH)₂/C (60 mg, 20% purity) and Pd/C (60 mg, 10% purity) under N₂ atmosphere. The suspension was degassed and purged with H₂ 3 times. The mixture was stirred under at 30° C. for 16 hr under H₂ (15 psi). The reaction mixture was filtered, then the filtrate was concentrated under reduced pressure and purified by prep-HPLC (flow: 25 mL/min; gradient: from 30-60% MeCN-water (0.1% TFA) over 7 min; column: 3_Phenomenex Luna C18 75×30 mm×3 um) to afford 1-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)phenyl)urea (Degrader 73, 27.79 mg, 40.41 μmol, 64% yield) as a white solid.

LCMS (ESI): m/z 688.2 [M+H]⁺

¹H NMR (400 MHz, d₆-DMSO) δ11.10 (m, 1H), 10.13-10.08 (m, 1H), 8.97-8.88 (m, 2H), 8.00 (s, 1H), 7.86 (d, J=8.8 Hz, 1H), 7.83 (d, J=8.8 Hz, 2H), 7.67 (d, J=8.8 Hz, 2H), 7.57 (s, 1H), 7.45-7.35 (m, 2H), 7.17 (d, J=8.0 Hz, 1H), 6.98 (s, 1H), 5.42-5.38 (m, 1H), 4.28 (s, 2H), 3.40 (s, 3H), 2.93-2.84 (m, 1H), 2.71-2.70 (m, 1H), 2.70-2.68 (m, 1H), 2.10-2.03 (m, 1H).

### 2-[1-[1-(2,6-dioxo-3-piperidyl)-6-fluoro-3-methyl-2-oxo-benzimidazol-5-yl]-4-hydroxy-4-piperidyl]-N-[5-fluoro-7-hydroxy-6-(1,1,4-trioxo-1,2,5-thiadiazolidin-2-yl)-2-naphthyl]acetamide (Degrader 74)

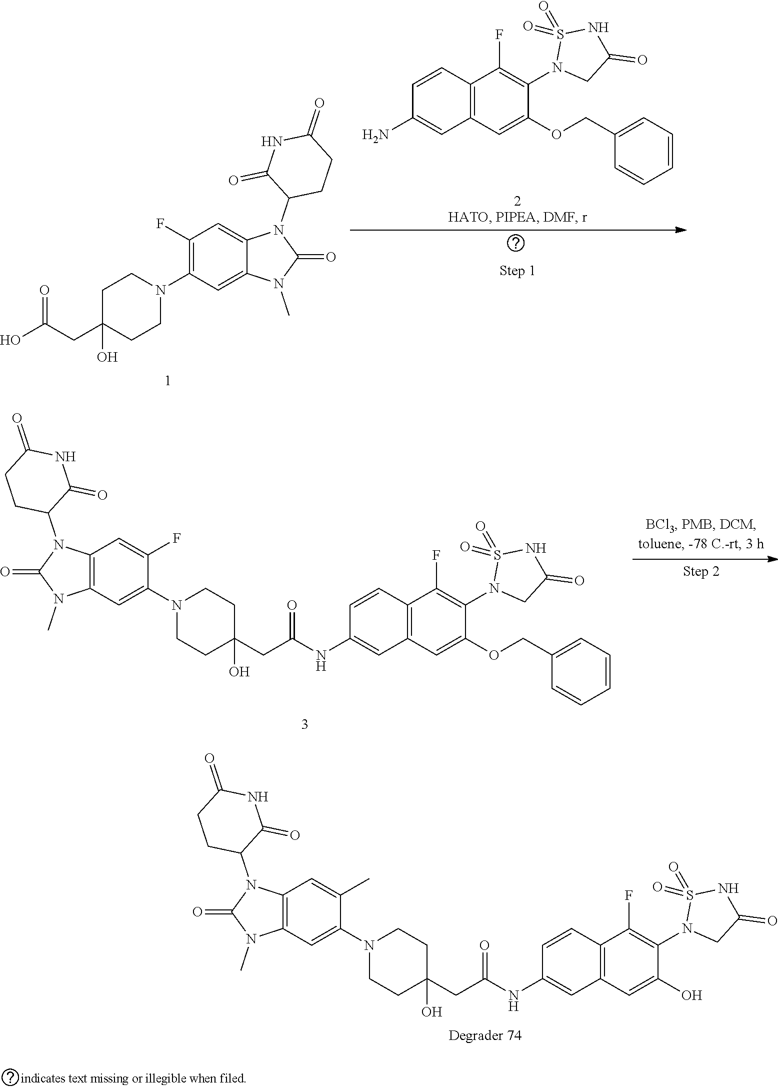 [SMILES: Cn1c(=O)n(C2CCC(=O)NC2=O)c2cc(F)c(N3CCC(O)(CC(=O)O)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2cc(F)c(N3CCC(O)(CC(=O)Nc4ccc5c(F)c(N6CC(=O)NS6(=O)=O)c(OCc6ccccc6)cc5c4)CC3)cc21 ; Cc1cc2c(cc1N1CCC(O)(CC(=O)Nc3ccc4c(F)c(N5CC(=O)NS5(=O)=O)c(O)cc4c3)CC1)n(C)c(=O)n2C1CCC(=O)NC1=O]

### Step 1: N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-6-fluoro-3-methyl-2-oxo-benzimidazol-5-yl]-4-hydroxy-4-piperidyl]acetamide (3)

Into a 25 mL single neck round-bottom flask containing a well-stirred solution of 2-[1-[1-(2,6-dioxo-3-piperidyl)-6-fluoro-3-methyl-2-oxo-benzimidazol-5-yl]-4-hydroxy-4-piperidyl]acetic acid (1, 50 mg, 113.95 μmol) and 5-(6-amino-3-benzyloxy-1-fluoro-2-naphthyl)-1,1-dioxo-1,2,5-thiadiazolidin-3-one (2, 45.74 mg, 113.95 μmol) in anhydrous DMF (2 mL) were added DIPEA (44.18 mg, 341.84 μmol, 59.54 μL) and HATU (64.99 mg, 170.92 μmol) at ambient temperature under nitrogen atmosphere. The reaction mixture was stirred at ambient temperature for 2 h. The reaction mixture was concentrated under reduced pressure and residue was treated with water (10 mL). The precipitate was filtered and dried. The crude compound was purified by reverse phase prep HPLC [Purification method: Column: Xbridge C18 (20×150) mm; Mobile phase A: 0.1% Formic acid in water; Mobile phase B: MeCN] to afford N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-6-fluoro-3-methyl-2-oxo-benzimidazol-5-yl]-4-hydroxy-4-piperidyl]acetamide (3, 40 mg, 43.53 μmol, 38% yield, Formic acid salt) as a colorless solid.

LCMS (ES+): m/z 818.4 [M+H]⁺

### Step 2: 2-[1-[1-(2,6-dioxo-3-piperidyl)-6-fluoro-3-methyl-2-oxo-benzimidazol-5-yl]-4-hydroxy-4-piperidyl]-N-[5-fluoro-7-hydroxy-6-(1,1,4-trioxo-1,2,5-thiadiazolidin-2-yl)-2-naphthyl]acetamide (Degrader 74)

Into a 20 mL single neck round-bottom flask containing a well-stirred solution of N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-6-fluoro-3-methyl-2-oxo-benzimidazol-5-yl]-4-hydroxy-4-piperidyl]acetamide (3, 40 mg, 43.53 μmol, formic acid salt) in a mixture of anhydrous DCM (2 mL) and toluene (2 mL) were added pentamethyl benzene (32.26 mg, 217.63 μmol) and 1.0 M solution of boron trichloride in DCM (1.09 mmol, 1.09 mL) at −78° C. The reaction mixture was stirred at ambient temperature. After 3 h, the reaction mixture was cooled to −78° C. and quenched with 5% MeOH in DCM (5 mL). The mixture was concentrated under reduced pressure and the reside was triturated with MTBE and filtered. The crude compound was purified by reverse phase prep HPLC [Purification method: Column: Xbridge C18 (20×150) mm; Mobile phase A: 0.1% TFA in water, B: MeCN] to afford 2-[1-[1-(2,6-dioxo-3-piperidyl)-6-fluoro-3-methyl-2-oxo-benzimidazol-5-yl]-4-hydroxy-4-piperidyl]-N-[5-fluoro-7-hydroxy-6-(1,1,4-trioxo-1,2,5-thiadiazolidin-2-yl)-2-naphthyl]acetamide (Degrader 74, 18 mg, 19.67 μmol, 45% yield, TFA salt) as a colorless solid.

LCMS (ES+): m/z 728.0 [M+H]⁺

¹H NMR (400 MHz, DMSO) δ 11.07 (s, 1H), 10.17 (s, 1H), 8.22-8.18 (m, 1H), 7.85 (d, J=8.9 Hz, 1H), 7.45 (dd, J=9.1, 2.0 Hz, 1H), 7.27-7.09 (m, 2H), 7.04-6.95 (m, 2H), 5.31 (dd, J=12.9, 5.4 Hz, 1H), 4.27 (s, 2H), 3.33 (s, 3H), 3.15-2.97 (m, 4H), 2.91-2.69 (m, 2H), 2.63-2.55 (m, 3H), 2.02-1.86 (m, 3H), 1.77 (d, J=12.7 Hz, 2H).

### 2-[4-[3-(2,6-dioxo-3-piperidyl)-1-methyl-2-oxo-8,9-dihydro-7H-imidazo[4,5-f]quinolin-6-yl]-1-piperidyl]-N-[5-fluoro-7-hydroxy-6-(1,1,4-trioxo-1,2,5-thiadiazolidin-2-yl)-2-naphthyl]acetamide (Degrader 75)

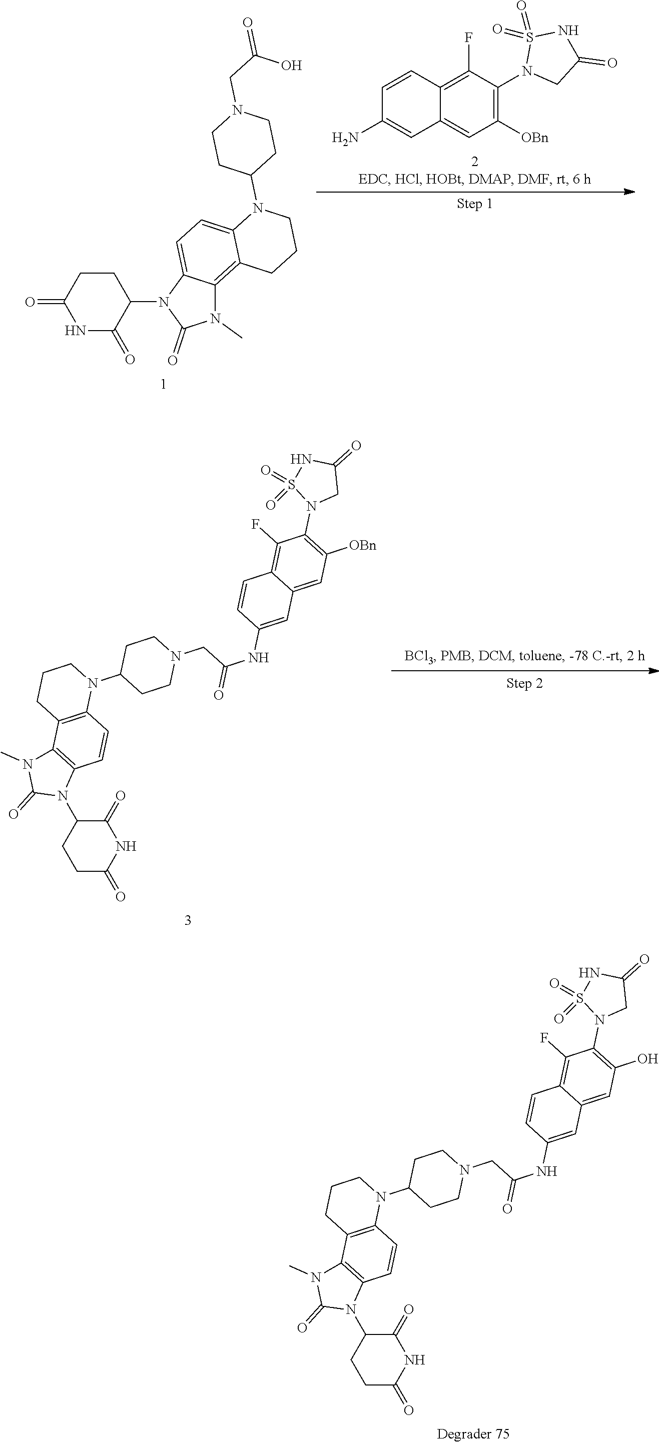 [SMILES: Cn1c(=O)n(C2CCC(=O)NC2=O)c2ccc3c(c21)CCCN3C1CCN(CC(=O)O)CC1 ; Nc1ccc2c(F)c(N3CC(=O)NS3(=O)=O)c(OCc3ccccc3)cc2c1 ; Cn1c(=O)n(C2CCC(=O)NC2=O)c2ccc3c(c21)CCCN3C1CCN(CC(=O)Nc2ccc3c(F)c(N4CC(=O)NS4(=O)=O)c(OCc4ccccc4)cc3c2)CC1 ; Cn1c(=O)n(C2CCC(=O)NC2=O)c2ccc3c(c21)CCCN3C1CCN(CC(=O)Nc2ccc3c(F)c(N4CC(=O)NS4(=O)=O)c(O)cc3c2)CC1]

### Step 1: N-[7-benzyloxy-5-fluoro-6-(1,1,4-trioxo-1,2,5-thiadiazolidin-2-yl)-2-naphthyl]-2-[4-[3-(2,6-dioxo-3-piperidyl)-1-methyl-2-oxo-8,9-dihydro-7H-imidazo[4,5-f]quinolin-6-yl]-1-piperidyl]acetamide (3)

Into a 50 mL single-neck round-bottom flask containing a well-stirred solution of 2-[4-[3-(2,6-dioxo-3-piperidyl)-1-methyl-2-oxo-8,9-dihydro-7H-imidazo[4,5-J]quinolin-6-yl]-1-piperidyl]acetic acid (1, 80 mg, 130.79 μmol, TFA salt) and 5-(6-amino-3-benzyloxy-1-fluoro-2-naphthyl)-1,1-dioxo-1,2,5-thiadiazolidin-3-one (2, 68.85 mg, 130.79 μmol, TFA salt) in anhydrous DMF (2 mL) were added EDC·HCl (74.83 mg, 392.37 μmol), HOBT (35.35 ng, 261.58 μmol) and DMAP (95.87 mg, 784.73 μmol). After 6 h, the solvent was removed and the residue was quenched with 1.5N HCl solution (50 mL). The precipitate was filtered and dried under reduced pressure to get N-[7-benzyloxy-5-fluoro-6-(1,1,4-trioxo-1,2,5-thiadiazolidin-2-yl)-2-naphthyl]-2-[4-[3-(2,6-dioxo-3-piperidyl)-1-methyl-2-oxo-8,9-dihydro-7H-imidazo[4,5-f]quinolin-6-yl]-1-piperidyl]acetamide (3, 120 mg, 90.61 μmol, 69% yield, HCl salt) as off white solid.

UPLC (ES+): m/z 838.0 [M+H]⁺

### Step 2: 2-[4-[3-(2,6-dioxo-3-piperidyl)-1-methyl-2-oxo-8,9-dihydro-7H-imidazo[4,5-f]quinolin-6-yl]-1-piperidyl]-N-[5-fluoro-7-hydroxy-6-(1,1,4-trioxo-1,2,5-thiadiazolidin-2-yl)-2-naphthyl]acetamide (Degrader 75)

Into a 50 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[4-[3-(2,6-dioxo-3-piperidyl)-1-methyl-2-oxo-8,9-dihydro-7H-imidazo[4,5-f]quinolin-6-yl]-1-piperidyl]acetamide (3, 120 mg, 90.48 μmol, HCl salt) and pentamethylbenzene (67.06 mg, 452.38 μmol) in anhydrous DCM (2 mL) and Toluene (2 mL) was added a 1.0 M solution of boron trichloride in DCM (1.81 mmol, 1.81 mL) at −78° C. The reaction mixture was then stirred at room temperature. After 2 h, the reaction mixture was quenched with 5% MeOH in DCM (2.5 mL) at −78° C. and the solvents were removed under reduced pressure. The crude compound was washed with MTBE (50 mL) and purified by reverse phase prep HPLC [Purification method: XSelect C18 (250×19) mm, 5 microns column; Mobile phase A: 0.1% formic acid in water; Mobile phase B: MeCN] to get 2-[4-[3-(2,6-dioxo-3-piperidyl)-1-methyl-2-oxo-8,9-dihydro-7H-imidazo[4,5-f]quinolin-6-yl]-1-piperidyl]-N-[5-fluoro-7-hydroxy-6-(1,1,4-trioxo-1,2,5-thiadiazolidin-2-yl)-2-naphthyl]acetamide (Degrader 75, 30 mg, 39.62 μmol, 44% yield) as an off-white solid.

LCMS (ES+): m/z 749.2 [M+H]⁺

¹HNMR (400 MHz, DMSO-d6): δ 11.05 (s, 1H), 10.76 (s, 1H), 9.82 (s, 1H), 9.78 (s, 1H), 8.11 (s, 1H), 7.90 (d, J=8.80 Hz, 1H), 7.47 (d, J=8.40 Hz, 1H), 7.00 (s, 1H), 6.77 (d, J=8.40 Hz, 1H), 6.57 (d, J=8.80 Hz, 1H), 5.28-5.23 (m, 1H), 4.19 (s, 2H), 4.08 (s, 2H), 4.03-3.96 (m, 1H), 3.66-3.62 (m, 2H), 3.55 (s, 3H), 3.15-3.08 (m, 5H), 2.90-2.84 (m, 1H), 2.68-2.64 (m, 2H), 2.16-2.08 (m, 2H), 1.98-1.83 (m, 5H).

### 2-[4-[1-(2,6-dioxo-3-piperidyl)-3-methyl-2-oxo-benzimidazol-5-yl]oxyphenyl]-N-[5-fluoro-7-hydroxy-6-(1,1,4-trioxo-1,2,5-thiadiazolidin-2-yl)-2-naphthyl]acetamide (Degrader 76)

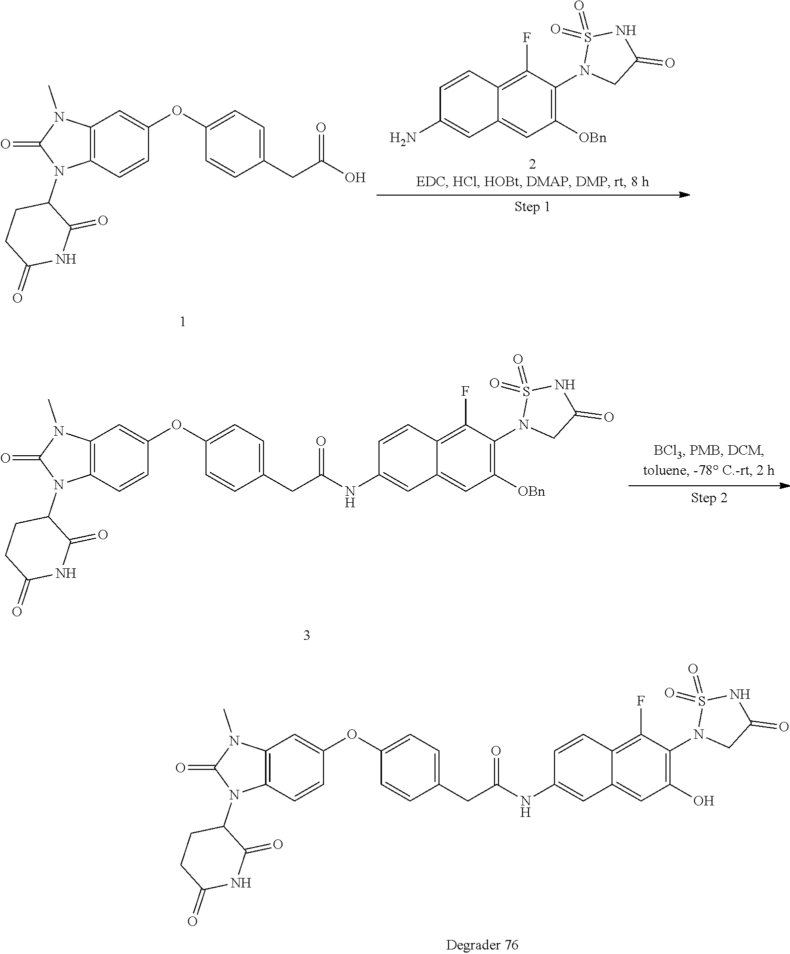 [SMILES: Cn1c(=O)n(C2CCC(=O)NC2=O)c2ccc(Oc3ccc(CC(=O)O)cc3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(Oc3ccc(CC(=O)Nc4ccc5c(F)c(N6CC(=O)NS6(=O)=O)c(OCc6ccccc6)cc5c4)cc3)cc21 ; Cn1c(=O)n(C2CCC(=O)NC2=O)c2ccc(Oc3ccc(CC(=O)Nc4ccc5c(F)c(N6CC(=O)NS6(=O)=O)c(O)cc5c4)cc3)cc21]

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]oxyphenyl]acetamide (3)

Into a 50 mL single-neck round-bottom flask a containing a well-stirred solution of 2-[4-[1-(2,6-dioxo-3-piperidyl)-3-methyl-2-oxo-benzimidazol-5-yl]oxyphenyl]acetic acid (1, 90 mg, 215.90 μmol) and 5-(6-amino-3-benzyloxy-1-fluoro-2-naphthyl)-1,1-dioxo-1,2,5-thiadiazolidin-3-one (2, 88.52 mg, 168.14 μmol, TFA Salt) in anhydrous DMF (2 mL) were added EDC·HCl (123.52 mg, 647.71 μmol), HOBT (58.35 mg, 431.81 μmol) and DMAP (158.26 mg, 1.30 mmol). The reaction mixture was stirred at room temperature. After 6 h, the solvent was removed from the reaction mixture and quenched with 1.5N HCl solution (50 mL). The precipitate was filtered and dried under reduced pressure to get N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]oxyphenyl]acetamide (3, 140 mg, 129.87 μmol, 60% yield) as an off-white solid.

UPLC (ES+): m/z 793.9 [M+H]⁺

### Step 2: 2-[4-[1-(2,6-dioxo-3-piperidyl)-3-methyl-2-oxo-benzimidazol-5-yl]oxyphenyl]-N-[5-fluoro-7-hydroxy-6-(1,1,4-trioxo-1,2,5-thiadiazolidin-2-yl)-2-naphthyl]acetamide (Degrader 76)

Into a 50 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]oxyphenyl]acetamide (3, 140 mg, 129.87 μmol), pentamethylbenzene (96.26 mg, 649.33 μmol) in anhydrous DCM (2 mL) and toluene (2 mL) was added a 1.0 M solution of boron trichloride in DCM (304.33 mg, 2.60 mmol, 2.60 mL) at −78° C. The reaction mixture was stirred at room temperature. After 2 h, the reaction mixture was quenched with 5% MeOH in DCM (3 mL) at −78° C. and excess solvents were removed under reduced pressure. The crude compound was washed with MTBE (50 mL) and purified by reverse phase column chromatography [Silicycle C18 column; Mobile phase A: 0.1% formic acid in water; Mobile phase B: MeCN] to get 2-[4-[1-(2,6-dioxo-3-piperidyl)-3-methyl-2-oxo-benzimidazol-5-yl]oxyphenyl]-N-[5-fluoro-7-hydroxy-6-(1,1,4-trioxo-1,2,5-thiadiazolidin-2-yl)-2-naphthyl]acetamide (Degrader 76, 75 mg, 104.03 μmol, 80% yield).

LCMS (ES+): m/z 703.0 [M+H]⁺

¹H NMR (400 MHz, DMSO-d6): δ 11.05 (s, 1H), 10.40 (s, 1H), 10.19 (s, 1H), 8.16 (s, 1H), 7.85 (d, J=8.80 Hz, 1H), 7.44 (dd, J=1.60, 9.20 Hz, 1H), 7.34 (d, J=8.80 Hz, 2H), 7.13 (d, J=8.40 Hz, 1H), 7.04 (d, J=2.40 Hz, 1H), 6.97-6.93 (m, 3H), 6.74 (dd, J=2.40, 8.60 Hz, 1H), 5.41-5.36 (m, 1H), 4.28 (s, 2H), 3.67 (s, 2H), 3.31 (s, 3H), 2.95-2.81 (m, 1H), 2.73-2.65 (m, 3H), 2.06-1.99 (m, 1H),

### N-(6-(1,1-dioxido-4-oxo-1,2,5-thiadiazolidin-2-yl)-5-fluoro-7-hydroxynaphthalen-2-yl)-2-(3-(1-(2,6-dioxopiperidin-3-yl)-3-methyl-2-oxo-2,3-dihydro-1H-benzo[d]imidazol-5-yl)phenyl)acetamide (Degrader 77)

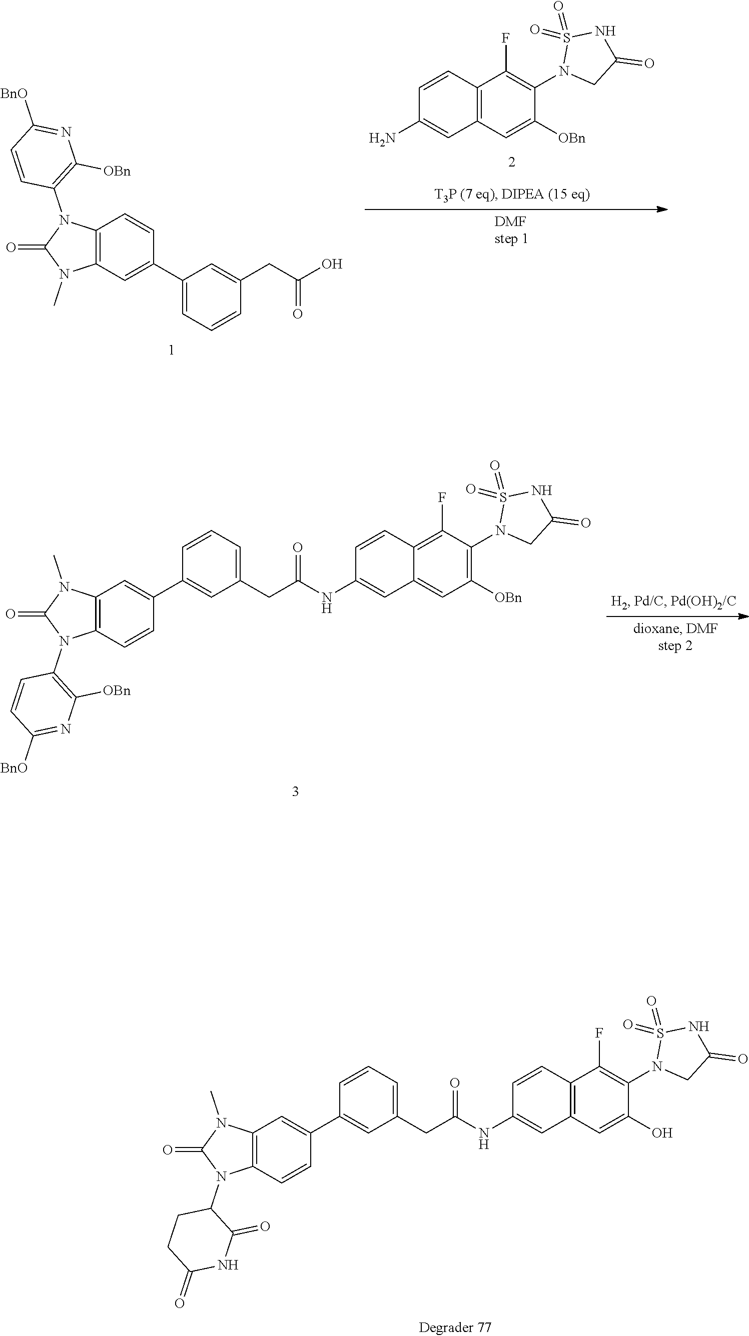 [SMILES: Cn1c(=O)n(-c2ccc(OCc3ccccc3)nc2OCc2ccccc2)c2ccc(-c3cccc(CC(=O)O)c3)cc21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(-c3cccc(CC(=O)Nc4ccc5c(F)c(N6CC(=O)NS6(=O)=O)c(OCc6ccccc6)cc5c4)c3)cc21 ; Cn1c(=O)n(C2CCC(=O)NC2=O)c2ccc(-c3cccc(CC(=O)Nc4ccc5c(F)c(N6CC(=O)NS6(=O)=O)c(O)cc5c4)c3)cc21]

### Step 1: N-(7-(benzyloxy)-6-(1,1-dioxido-4-oxo-1,2,5-thiadiazolidin-2-yl)-5-fluoronaphthalen-2-yl)-2-(3-(1-(2,6-bis(benzyloxy)pyridin-3-yl)-3-methyl-2-oxo-2,3-dihydro-1H-benzo[d]imidazol-5-yl)phenyl)acetamide (3)

To a solution of 2-(3-(1-(2,6-bis(benzyloxy)pyridin-3-yl)-3-methyl-2-oxo-2,3-dihydro-1H-benzo[d]imidazol-5-yl)phenyl)acetic acid (1, 200 mg, 349.88 μmol) and 5-(6-amino-3-(benzyloxy)-1-fluoronaphthalen-2-yl)-1,2,5-thiadiazolidin-3-one 1,1-dioxide (2, 140.45 mg, 349.88 μmol) in DMF (4 mL) was added DIEA (678.30 mg, 5.25 mmol, 914.15 μL, 10 eq) and T₃P (1.56 g, 2.45 mmol, 50% purity in ethyl acetate). The mixture was stirred at 50° C. for 16 h. The reaction mixture was concentrated under reduced pressure to remove DMF. The residue was purified by prep-HPLC (flow: 25 mL/min; gradient: from 33-63% MeCN-water (0.05% ammonia hydroxide v/v) over 10 min; column: Waters Xbridge 150×25 mm×5 um) to afford N-(7-(benzyloxy)-6-(1,1-dioxido-4-oxo-1,2,5-thiadiazolidin-2-yl)-5-fluoronaphthalen-2-yl)-2-(3-(1-(2,6-bis(benzyloxy)pyridin-3-yl)-3-methyl-2-oxo-2,3-dihydro-1H-benzo[d]imidazol-5-yl)phenyl)acetamide (3, 130 mg, 130.24 μmol, 37% yield) as a white solid.

LCMS (ES+): m/z 955.2[M+H]⁺

### Step 2: N-(6-(1,1-dioxido-4-oxo-1,2,5-thiadiazolidin-2-yl)-5-fluoro-7-hydroxynaphthalen-2-yl)-2-(3-(1-(2,6-dioxopiperidin-3-yl)-3-methyl-2-oxo-2,3-dihydro-1H-benzo[d]imidazol-5-yl)phenyl)acetamide (Degrader 77)

To a solution of N-(7-(benzyloxy)-6-(1,1-dioxido-4-oxo-1,2,5-thiadiazolidin-2-yl)-5-fluoronaphthalen-2-yl)-2-(3-(1-(2,6-bis(benzyloxy)pyridin-3-yl)-3-methyl-2-oxo-2,3-dihydro-1H-benzo[d]imidazol-5-yl)phenyl)acetamide (3, 120 mg, 125.65 μmol) in DMF (2 mL) were added Pd(OH)₂/C (50 mg, wet, 10% purity) and Pd/C (50 mg, wet, 10% purity). The suspension was degassed and purged with H₂ for 3 times. The mixture was stirred under H₂ (15 psi) at 30° C. for 16 h. The mixture was filtered through Celite. The reaction mixture was concentrated under reduced pressure to remove DMF. The residue was purified by prep-HPLC (FlowRate: 25 ml/min; gradient: from 33%-63% MeCN in water (0.1% TFA) over 9 min; Column: 3_Phenomenex Luna C18 75×30 mm×3 um) to afford N-(6-(1,1-dioxido-4-oxo-1,2,5-thiadiazolidin-2-yl)-5-fluoro-7-hydroxynaphthalen-2-yl)-2-(3-(1-(2,6-dioxopiperidin-3-yl)-3-methyl-2-oxo-2,3-dihydro-1H-benzo[d]imidazol-5-yl)phenyl)acetamide (Degrader 77, 36.53 mg, 52.56 μmol, 42% yield) as a white solid.

LCMS (ESI): m/z 687.1[M+H]⁺

¹H NMR (400 MHz, DMSO-d6) δ=11.13 (s, 1H), 10.46 (s, 1H), 10.40-10.31 (m, 1H), 8.19 (s, 1H), 7.87 (d, J=8.9 Hz, 1H), 7.69 (s, 1H), 7.60 (br d, J=7.8 Hz, 1H), 7.53-7.40 (m, 3H), 7.38-7.31 (m, 2H), 7.22 (d, J=8.2 Hz, 1H), 6.96 (s, 1H), 5.42 (dd, J=5.4, 12.8 Hz, 1H), 4.38 (br s, 2H), 3.80 (s, 2H), 3.42 (s, 3H), 2.99-2.89 (m, 1H), 2.82-2.67 (m, 2H), 2.13-2.00 (m, 1H)

### 3-[5-[1-[(E)-3-[5-fluoro-7-hydroxy-6-(1,1,4-trioxo-1,2,5-thiadiazolidin-2-yl)-2-naphthyl]allyl]-4-piperidyl]-3-methyl-2-oxo-benzimidazol-1-yl]piperidine-2,6-dione (Degrader 78)

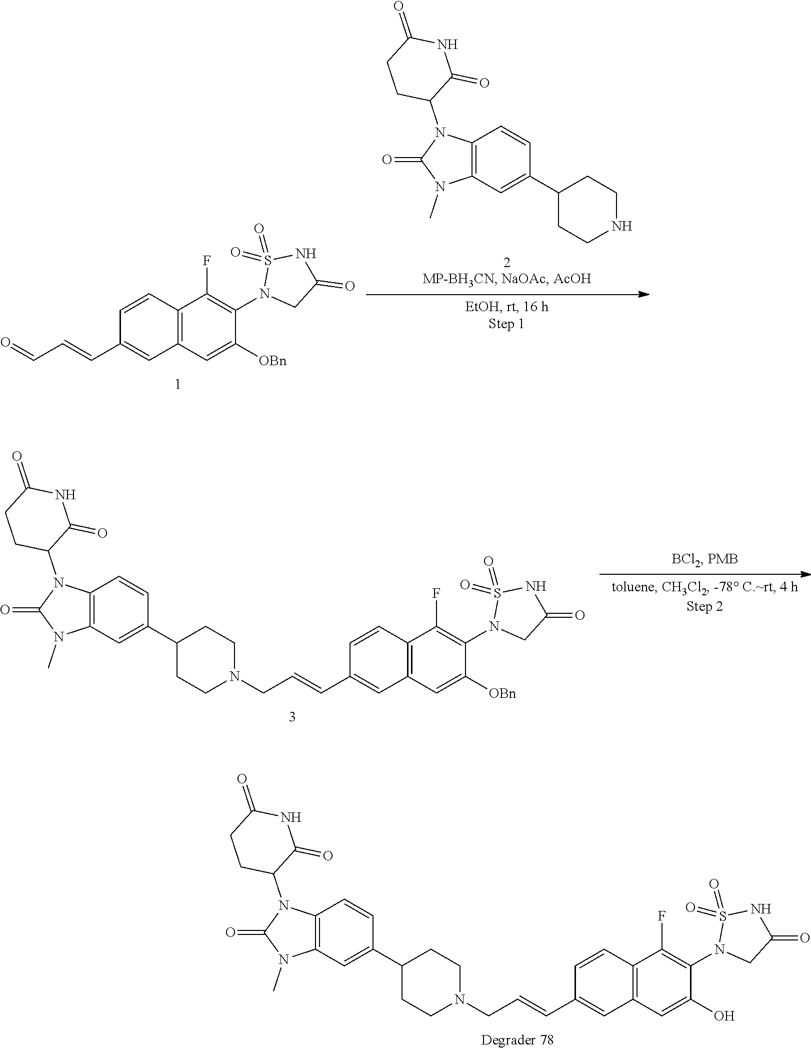 [SMILES: O=C/C=C/c1ccc2c(F)c(N3CC(=O)NS3(=O)=O)c(OCc3ccccc3)cc2c1 ; Cn1c(=O)n(C2CCC(=O)NC2=O)c2ccc(C3CCNCC3)cc21 ; Cn1c(=O)n(C2CCC(=O)NC2=O)c2ccc(C3CCN(C/C=C/c4ccc5c(F)c(N6CC(=O)NS6(=O)=O)c(OCc6ccccc6)cc5c4)CC3)cc21 ; Cn1c(=O)n(C2CCC(=O)NC2=O)c2ccc(C3CCN(C/C=C/c4ccc5c(F)c(N6CC(=O)NS6(=O)=O)c(O)cc5c4)CC3)cc21]

### Step 1: 3-[5-[1-[(E)-3-[7-benzyloxy-5-fluoro-6-(1,1,4-trioxo-1,2,5-thiadiazolidin-2-yl)-2-naphthyl]allyl]-4-piperidyl]-3-methyl-2-oxo-benzimidazol-1-yl]piperidine-2,6-dione (3)

To a stirred solution of (E)-3-[7-benzyloxy-5-fluoro-6-(1,1,4-trioxo-1,2,5-thiadiazolidin-2-yl)-2-naphthyl]prop-2-enal (1, 500 mg, 334.66 μmol) in DMSO (5 mL) was added 3-[3-methyl-2-oxo-5-(4-piperidyl)benzimidazol-1-yl]piperidine-2,6-dione (2, 153.68 mg, 401.60 μmol, HCl salt) at 0° C. The reaction mixture was stirred at the same temperature for 15 mins. Thereafter, MP-BH₃CN (1.0 g/2 mmol loading, 334 mg, 669.33 μmol) was added to the reaction mixture and stirring was continued at room temperature for 16 h. The reaction mixture filtered through Celite and washed with DCM (10 mL). The filtrate was concentrated under reduced pressure to get a residue to which ice-cold water (5 mL) was added to precipitate a solid that was filtered and dried to afford 3-[5-[1-[(E)-3-[7-benzyloxy-5-fluoro-6-(1,1,4-trioxo-1,2,5-thiadiazolidin-2-yl)-2-naphthyl]allyl]-4-piperidyl]-3-methyl-2-oxo-benzimidazol-1-yl]piperidine-2,6-dione (3, 350 mg, 262.17 μmol, 78% yield) LCMS (ES+): m/z 767.2 [M+H]⁺

### Step 2: 3-[5-[1-[(E)-3-[5-fluoro-7-hydroxy-6-(1,1,4-trioxo-1,2,5-thiadiazolidin-2-yl)-2-naphthyl]allyl]-4-piperidyl]-3-methyl-2-oxo-benzimidazol-1-yl]piperidine-2,6-dione (Degrader 78)

Into a 50 mL two-neck round-bottom flask containing a well-stirred solution of 3-[5-[1-[(E)-3-[7-benzyloxy-5-fluoro-6-(1,1,4-trioxo-1,2,5-thiadiazolidin-2-yl)-2-naphthyl]allyl]-4-piperidyl]-3-methyl-2-oxo-benzimidazol-1-yl]piperidine-2,6-dione (3, 350 mg, 262.17 μmol) in toluene (5 mL) and CH₂Cl₂ (5 mL) under inert atmosphere was added pentamethylbenzene (194.33 mg, 1.31 mmol) at room temperature. The mixture was cooled to −78° C. and BCl₃ solution (1M solution in DCM, 5.24 mmol, 5.24 mL) was added slowly to the reaction mixture. The reaction mixture was gradually warmed to room temperature and stirred for 4 h. The reaction mixture was slowly quenched with 5% CH₃OH in CH₂Cl₂ (10 mL) solution at −78° C. and warmed to room temperature. Evaporation under vacuum and trituration with MTBE (2×50 mL) gave the crude compound that was purified by reverse-phase preparatory HPLC [Column: x-select C18 (250×19) mm 5 micron, Mobile phase A: 0.1% formic acid in water and Mobile Phase B: CH₃CN] afforded 3-[5-[1-[(E)-3-[5-fluoro-7-hydroxy-6-(1,1,4-trioxo-1,2,5-thiadiazolidin-2-yl)-2-naphthyl]allyl]-4-piperidyl]-3-methyl-2-oxo-benzimidazol-1-yl]piperidine-2,6-dione (Degrader 78, 45.69 mg, 62.22 μmol, 24% yield, formic acid salt) as a white powder.

¹H-NMR (400 MHz, DMSO-d6): δ 11.08 (s, 1H), 9.86 (s, 1H), 9.40 (brs, 1H), 7.91 (d, J=8.40 Hz, 1H), 7.82 (s, 1H), 7.64 (d, J=8.80 Hz, 1H), 7.11-6.92 (m, 5H), 6.61-6.53 (m, 1H), 5.36 (dd, J=5.60, 12.80 Hz, 1H), 4.11 (s, 2H), 4.05-3.95 (m, 1H), 3.72-3.55 (m, 1H), 3.35 (s, 3H), 3.21-3.08 (m, 2H), 3.31-2.87 (m, 2H), 2.78-2.60 (m, 3H), 2.08-1.89 (m, 6H).

LCMS (ES+): m/z 677.2 [M+H]⁺

### N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-2-methylphenyl)acetamide (Degrader 79)

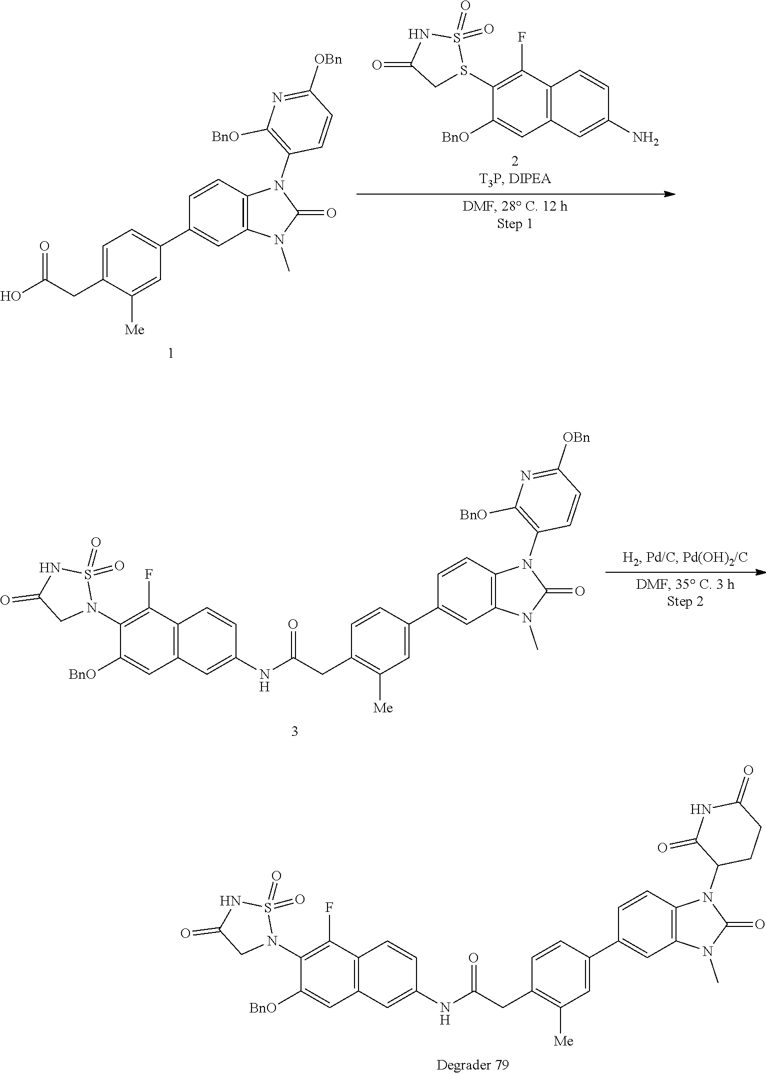

### Step 1: N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-2-methylphenyl)acetamide (3)

To a solution of 2-(4-(1-(2,6-bis(benzyloxy)pyridin-3-yl)-3-methyl-2-oxo-2,3-dihydro-1H-benzo[d]imidazol-5-yl)-2-methylphenyl)acetic acid (1, 0.2 g, 341.50 μmol), 5-(6-amino-3-(benzyloxy)-1-fluoronaphthalen-2-yl)-1,2,5-thiadiazolidin-3-one 1,1-dioxide (143.94 mg, 358.58 μmol) and N-ethyl-N-isopropyl-propan-2-amine (2, 441.37 mg, 3.42 mmol, 594.84 μL) in DMF (2 mL) was added T₃P (1.52 g, 2.39 mmol, 1.42 mL, 50% purity) and then the mixture was stirred at 25° C. for 12 hrs. The reaction mixture was purified by prep-HPLC (Column: Waters Xbridge 150*25 mm*5 um 32%-62% ACN in water (0.05% ammonia hydroxide v/v)-ACN FlowRate (ml/min): 25). Compound N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-2-methylphenyl)acetamide (3, 130 mg, 123.42 μmol, 36% yield) as a yellow solid.

LCMS (ES+): m/z 969.2 [M+H]⁺

¹H NMR (400 MHz, DMSO-d6) δ=10.43 (s, 1H), 8.27 (s, 1H), 8.24-8.11 (m, 1H), 7.93-7.80 (m, 2H), 7.61-7.51 (m, 5H), 7.50-7.43 (m, 3H), 7.42-7.33 (m, 6H), 7.33-7.21 (m, 7H), 7.19 (s, 1H), 6.75 (d, J=8.0 Hz, 1H), 6.63 (d, J=8.4 Hz, 1H), 5.46-5.32 (m, 4H), 5.25 (s, 2H), 4.06 (s, 2H), 3.79 (s, 2H), 3.46 (s, 3H), 2.41 (s, 3H)

### Step 2: N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-2-methylphenyl)acetamide (Degrader 79)

N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-2-methylphenyl)acetamide (3, 110 mg, 113.51 μmol) in DMF (4 mL) was added Pd(OH)₂ (100 mg, 142.42 μmol, 20% purity) and Pd/C (100 mg, 84.44 μmol, 10% purity). The suspension was degassed and purged with H₂ for 3 times. The mixture was stirred at H₂ (15 psi) at 35° C. for 3 hrs. The mixture was filtered to get filtrate and concentrated under vacuum to afford the crude product. The residue was purified by prep-HPLC (Column: 3_Phenomenex Luna C18 75*30 mm*3 um, 33%-63% ACN in water (0.1% TFA)-ACN over 7 min FlowRate: 25 ml/min). Compound N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-2-methylphenyl)acetamide (Degrader 79, 66.65 mg, 81.81 μmol, 72% yield, TFA salt) as a white solid.

¹H NMR (400 MHz, DMSO) δ 11.11 (s, 1H), 10.42 (s, 1H), 8.20-8.16 (m, 1H), 7.87 (d, J=9.0 Hz, 1H), 7.56-7.52 (m, 1H), 7.51-7.45 (m, 3H), 7.38-7.32 (m, 2H), 7.18 (d, J=8.3 Hz, 1H), 6.96 (s, 1H), 5.40 (dd, J=12.8, 5.4 Hz, 1H), 4.40 (s, 2H), 3.79 (s, 2H), 3.41 (s, 3H), 2.99-2.86 (m, 1H), 2.81-2.70 (m, 1H), 2.68-2.59 (m, 1H), 2.40 (s, 3H), 2.09-2.00 (m, 1H).

### 3-[3-[1-(2,6-dioxo-3-piperidyl)-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]propanamide (Degrader 80)

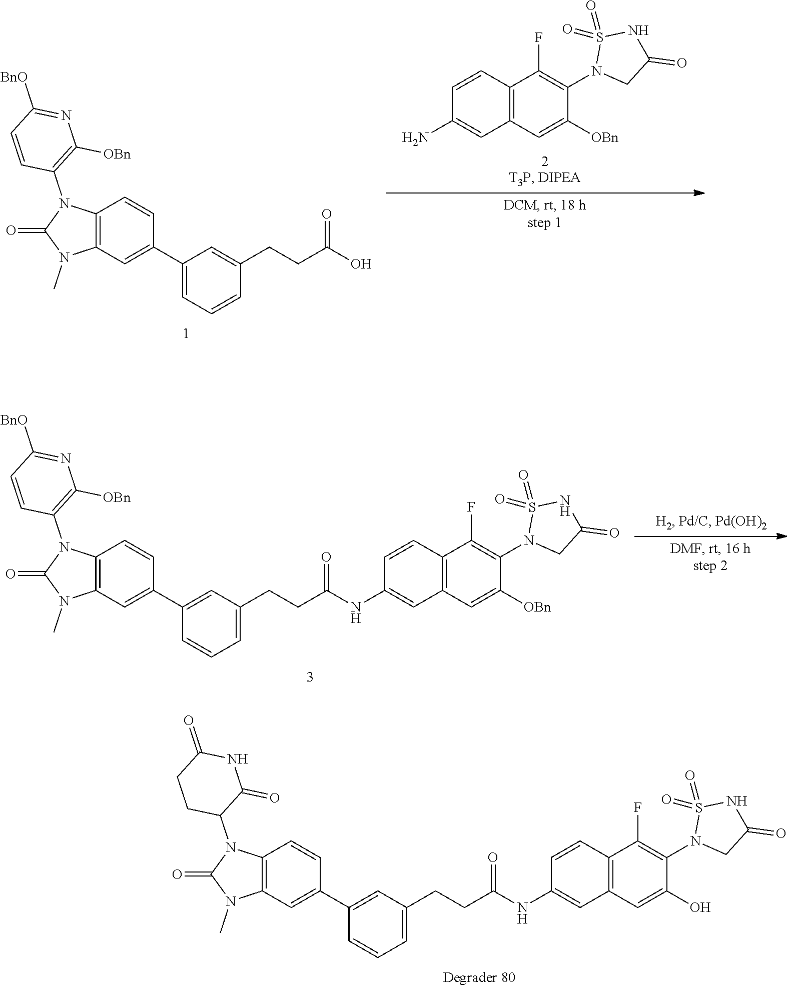 [SMILES: Cn1c(=O)n(-c2ccc(OCc3ccccc3)nc2OCc2ccccc2)c2ccc(-c3cccc(CCC(=O)O)c3)cc21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(-c3cccc(CCC(=O)Nc4ccc5c(F)c(N6CC(=O)NS6(=O)=O)c(OCc6ccccc6)cc5c4)c3)cc21 ; Cn1c(=O)n(C2CCC(=O)NC2=O)c2ccc(-c3cccc(CCC(=O)Nc4ccc5c(F)c(N6CC(=O)NS6(=O)=O)c(O)cc5c4)c3)cc21]

### Step 1: N-(7-(benzyloxy)-6-(1,1-dioxido-4-oxo-1,2,5-thiadiazolidin-2-yl)-5-fluoronaphthalen-2-yl)-3-(3-(1-(2,6-bis(benzyloxy)pyridin-3-yl)-3-methyl-2-oxo-2,3-dihydro-1H-benzo[d]imidazol-5-yl)phenyl)propanamide (3)

To a solution of 3-(3-(1-(2,6-bis(benzyloxy)pyridin-3-yl)-3-methyl-2-oxo-2,3-dihydro-1H-benzo[d]imidazol-5-yl)phenyl)propanoic acid (1, 0.17 g, 290.28 μmol) and 5-(6-amino-3-(benzyloxy)-1-fluoronaphthalen-2-yl)-1,2,5-thiadiazolidin-3-one 1,1-dioxide (2, 116.52 mg, 290.28 μmol) in DCM (15 mL) was added T₃P (50 wt % in EtOAc) (2.77 g, 8.71 mmol) and DIPEA (562.74 mg, 4.35 mmol, 758.42 uL). The mixture was stirred at 20° C. for 16 h. The mixture was washed with brine (30 mL×2), dried over Na₂SO₄, filtered and concentrated under reduced pressure. The residue was purified by prep-HPLC (flow: 60 mL/min; gradient: from 28-58% MeCN in water (0.05% ammonia hydroxide) over 10 min; column: Waters Xbridge 150×25 mm×5 um) to afford N-(7-(benzyloxy)-6-(1,1-dioxido-4-oxo-1,2,5-thiadiazolidin-2-yl)-5-fluoronaphthalen-2-yl)-3-(3-(1-(2,6-bis(benzyloxy)pyridin-3-yl)-3-methyl-2-oxo-2,3-dihydro-1H-benzo[d]imidazol-5-yl)phenyl)propanamide (3, 55 mg, 56.76 μmol, 20% yield) as a white solid.

LCMS (ESI): m/z 969.2 [M+H]⁺

### Step 2: 3-[3-[1-(2,6-dioxo-3-piperidyl)-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]propanamide (Degrader 80)

To a solution of N-(7-(benzyloxy)-6-(1,1-dioxido-4-oxo-1,2,5-thiadiazolidin-2-yl)-5-fluoronaphthalen-2-yl)-3-(3-(1-(2,6-bis(benzyloxy)pyridin-3-yl)-3-methyl-2-oxo-2,3-dihydro-1H-benzo[d]imidazol-5-yl)phenyl)propanamide (3, 0.06 g, 61.92 μmol) in DMF (3 mL) were added Pd/C (601.58 mg, 495.33 μmol, 10% purity) and Pd(OH)₂ (347.81 mg, 495.33 μmol, 20% purity). The mixture was stirred at 20° C. for 16 hr under H₂ (15 psi). The mixture was filtered and washed with DMF (1 mL). The filtrated was purified by prep-HPLC (flow: 28 mL/min; gradient: from 38-60% MeCN in water (0.1% TFA) over 9 min; column: Phenomenex Synergi C18 150×25 mm×10 um) to afford 3-[3-[1-(2,6-dioxo-3-piperidyl)-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]propanamide (Degrader 80, 29.98 mg, 36.80 μmol, 59% yield, TFA salt) as a yellow solid.

LCMS (ESI): m/z 701.2 [M+H]⁺

¹H NMR (400 MHz, DMSO-d₆) δ11.11 (s, 1H), 10.17 (s, 1H), 10.19 (s, 1H), 9.98-9.95 (s, 1H), 8.16 (s, 1H), 7.83-7.81 (m, 2H), 7.60-7.50 (m, 1H), 7.47-7.25 (m, 4H), 7.17-6.95 (m, 2H), 5.42-5.37 (m, 1H), 4.19 (s, 2H), 3.38 (s, 3H), 3.04-3.01 (m, 2H), 2.89-2.62 (m, 5H), 2.06-2.03 (m, 1H).

### N-(6-(1,1-dioxido-4-oxo-1,2,5-thiadiazolidin-2-yl)-5-fluoro-7-hydroxynaphthalen-2-yl)-2-(4-((1-(2,6-dioxopiperidin-3-yl)-3-methyl-2-oxo-2,3-dihydro-1H-benzo[d]imidazol-5-yl)oxy)-3-methylphenyl)acetamide (Degrader 81)

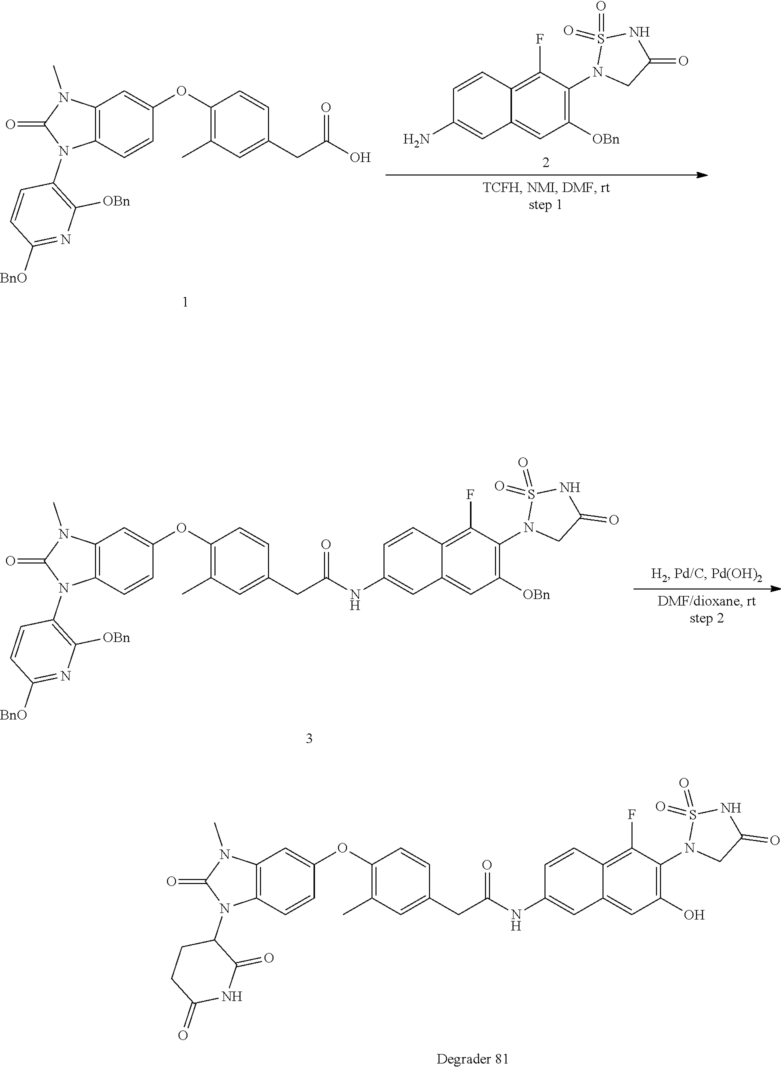 [SMILES: Cc1cc(CC(=O)O)ccc1Oc1ccc2c(c1)n(C)c(=O)n2-c1ccc(OCc2ccccc2)nc1OCc1ccccc1 ; Nc1ccc2c(F)c(N3CC(=O)NS3(=O)=O)c(OCc3ccccc3)cc2c1 ; Cc1cc(CC(=O)Nc2ccc3c(F)c(N4CC(=O)NS4(=O)=O)c(OCc4ccccc4)cc3c2)ccc1Oc1ccc2c(c1)n(C)c(=O)n2-c1ccc(OCc2ccccc2)nc1OCc1ccccc1 ; Cc1cc(CC(=O)Nc2ccc3c(F)c(N4CC(=O)NS4(=O)=O)c(O)cc3c2)ccc1Oc1ccc2c(c1)n(C)c(=O)n2C1CCC(=O)NC1=O]

### Step 1: N-(7-(benzyloxy)-6-(1,1-dioxido-4-oxo-1,2,5-thiadiazolidin-2-yl)-5-fluoronaphthalen-2-yl)-2-(4-((1-(2,6-bis(benzyloxy)pyridin-3-yl)-3-methyl-2-oxo-2,3-dihydro-1H-benzo[d]imidazol-5-yl)oxy)-3-methylphenyl)acetamide (3)

To a solution of 2-(4-((1-(2,6-bis(benzyloxy)pyridin-3-yl)-3-methyl-2-oxo-2,3-dihydro-1H-benzo[d]imidazol-5-yl)oxy)-3-methylphenyl)acetic acid (1, 80 mg, 132.97 μmol) and 5-(6-amino-3-benzyloxy-1-fluoro-2-naphthyl)-1,1-dioxo-1,2,5-thiadiazolidin-3-one (2, 58.71 mg, 146.27 μmol) in DMF (2.5 mL) was added [chloro(dimethylamino)methylene]-dimethyl-ammonium; hexafluorophosphate (93.27 mg, 332.42 μmol) and 1-methylimidazole (54.59 mg, 664.84 μmol, 53 uL). The mixture was stirred at 25° C. for 12 h. The reaction mixture was filtered to get the filtrate. The filtrate was purified by prep-HPLC (base condition; flow: 25 mL/min; gradient: from 35-65% water (0.05% ammonia hydroxide v/v)-ACN over 10 min; column: Waters Xbridge 150*25 mm*5 um) to afford N-(7-(benzyloxy)-6-(1,1-dioxido-4-oxo-1,2,5-thiadiazolidin-2-yl)-5-fluoronaphthalen-2-yl)-2-(4-((1-(2,6-bis(benzyloxy)pyridin-3-yl)-3-methyl-2-oxo-2,3-dihydro-1H-benzo[d]imidazol-5-yl)oxy)-3-methylphenyl)acetamide (3, 25 mg, 25.38 μmol, 19% yield) as a white solid.

LCMS (ESI): m/z 985.6 [M+H]⁺

### Step 2: N-(6-(1,1-dioxido-4-oxo-1,2,5-thiadiazolidin-2-yl)-5-fluoro-7-hydroxynaphthalen-2-yl)-2-(4-((1-(2,6-dioxopiperidin-3-yl)-3-methyl-2-oxo-2,3-dihydro-1H-benzo[d]imidazol-5-yl)oxy)-3-methylphenyl)acetamide (Degrader 81)

To a solution of N-(7-(benzyloxy)-6-(1,1-dioxido-4-oxo-1,2,5-thiadiazolidin-2-yl)-5-fluoronaphthalen-2-yl)-2-(4-((1-(2,6-bis(benzyloxy)pyridin-3-yl)-3-methyl-2-oxo-2,3-dihydro-1H-benzo[d]imidazol-5-yl)oxy)-3-methylphenyl)acetamide (3, 50 mg, 50.76 μmol, 1 eq) in DMF (1 mL) and dioxane (0.1 mL) were added Pd/C (50.00 mg, 41.17 μmol, 10% purity) and Pd(OH)₂/C (50 mg, 76.14 μmol, 10% purity) under N₂ atmosphere. The suspension was degassed and purged with H₂ 3 times. The mixture was stirred under H₂ (15 Psi) at 35° C. for 12 h. The reaction mixture was filtered to get the filtrate. The filtrate was purified by prep-HPLC (TFA condition; flow: 25 mL/min; gradient: from 35-65% water (0.1% TFA)-ACN over 7 min; column: 3_Phenomenex Luna C18 75×30 mm×3 um) to afford N-(6-(1,1-dioxido-4-oxo-1,2,5-thiadiazolidin-2-yl)-5-fluoro-7-hydroxynaphthalen-2-yl)-2-(4-((1-(2,6-dioxopiperidin-3-yl)-3-methyl-2-oxo-2,3-dihydro-1H-benzo[d]imidazol-5-yl)oxy)-3-methylphenyl)acetamide (Degrader 81, 17.98 mg, 21.43 μmol, 42% yield) as a yellow solid.

LCMS (ESI): m/z 717.1 [M+H]+

¹H NMR (400 MHz, DMSO) δ 11.09 (s, 1H), 10.36 (s, 1H), 9.97 (s, 1H), 8.15 (s, 1H), 7.84 (d, J=9.0 Hz, 1H), 7.46-7.41 (m, 1H), 7.30-7.27 (m, 1H), 7.17-7.13 (m, 1H), 7.07 (d, J=8.5 Hz, 1H), 6.94 (d, J=2.1 Hz, 2H), 6.76 (d, J=8.3 Hz, 1H), 6.59 (dd, J=8.5, 2.4 Hz, 1H), 5.35 (dd, J=12.7, 5.4 Hz, 1H), 4.19 (s, 2H), 3.64 (s, 2H), 3.29 (s, 3H), 2.97-2.82 (m, 1H), 2.75-2.57 (m, 2H), 2.24 (s, 3H), 2.05-1.97 (m, 1H).

### N-(6-(1,1-dioxido-4-oxo-1,2,5-thiadiazolidin-2-yl)-5-fluoro-7-hydroxynaphthalen-2-yl)-6-(1-(2,6-dioxopiperidin-3-yl)-3-methyl-2-oxo-2,3-dihydro-1H-benzo[d]imidazol-5-yl)-1-methyl-1H-indole-3-carboxamide (Degrader 82)

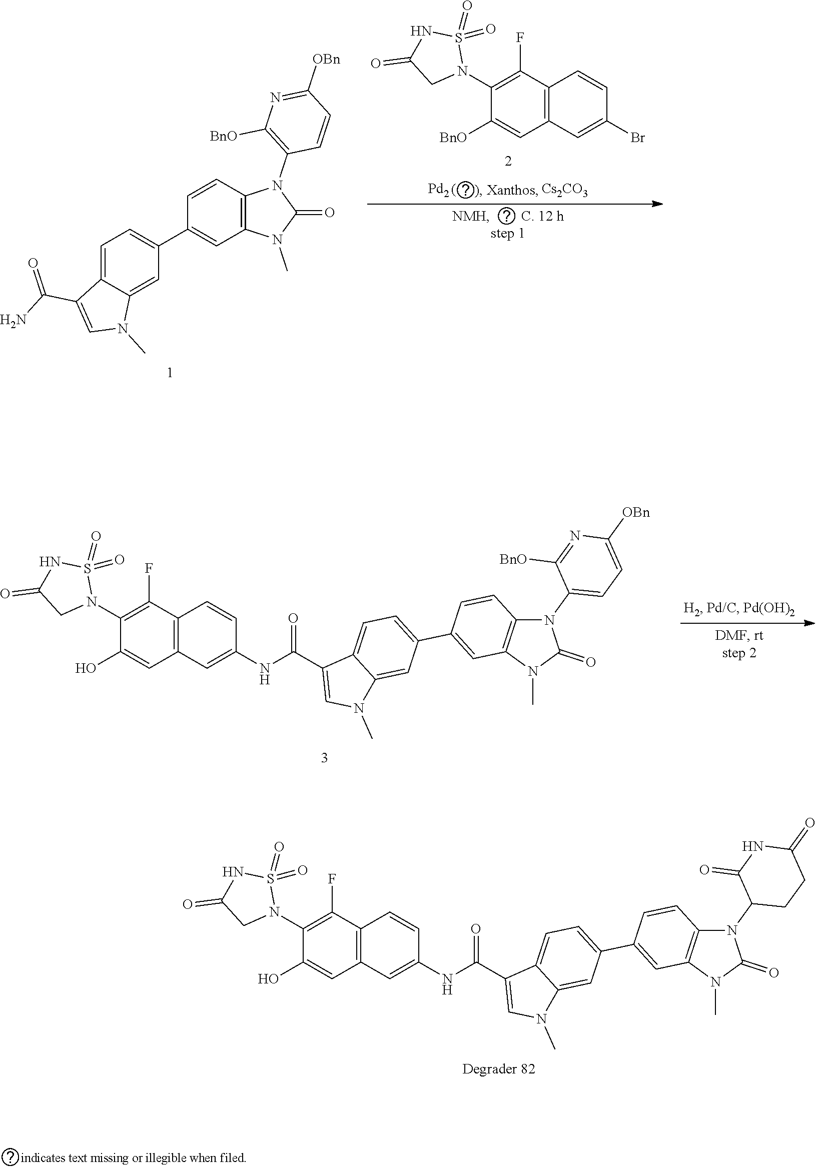

### Step 1: N-(7-(benzyloxy)-6-(1,1-dioxido-4-oxo-1,2,5-thiadiazolidin-2-yl)-5-fluoronaphthalen-2-yl)-6-(1-(2,6-bis(benzyloxy)pyridin-3-yl)-3-methyl-2-oxo-2,3-dihydro-1H-benzo[d]imidazol-5-yl)-1-methyl-1H-indole-3-carboxamide (3)

To a mixture of 6-(1-(2,6-bis(benzyloxy)pyridin-3-yl)-3-methyl-2-oxo-2,3-dihydro-1H-benzo[d]imidazol-5-yl)-1-methyl-1H-indole-3-carboxamide (1, 100 mg, 164.02 μmol), 5-(3-(benzyloxy)-6-bromo-1-fluoronaphthalen-2-yl)-1,2,5-thiadiazolidin-3-one 1,1-dioxide (2, 152.64 mg, 328.05 μmol) in NMP (5 mL) was added cesium carbonate (106.88 mg, 328.05 μmol), Xantphos (18.98 mg, 32.80 μmol) and (1E,4E)-1,5-diphenylpenta-1,4-dien-3-one; palladium (15.02 mg, 16.40 μmol). The mixture was degassed and purged with N₂ for 3 times, and then the mixture was stirred at 110° C. for 12 h. The reaction mixture was diluted with H₂O (10 mL) and DCM (10 mL), brown precipitate was formed, the mixture was filtered through Celite, then the filter cake was washed with MeOH (10 mL*3) and the filter liquor was concentrated under reduced pressure to give the crude product. The crude product was purified by reversed-phase HPLC (40 g of XB-C18, 20-35 M, 100 Å) Mobile phase: A for H₂O (0.1% FA v/v) and B for acetonitrile; Gradient: B 0%-30% in 35 min; Flow rate: 60 ml/min; Column temperature: R.T. Wavelength: 220 nm/254 nmu) to afford N-(7-(benzyloxy)-6-(1,1-dioxido-4-oxo-1,2,5-thiadiazolidin-2-yl)-5-fluoronaphthalen-2-yl)-6-(1-(2,6-bis(benzyloxy)pyridin-3-yl)-3-methyl-2-oxo-2,3-dihydro-1H-benzo[d]imidazol-5-yl)-1-methyl-1H-indole-3-carboxamide (3, 30 mg, 30.18 μmol, 18% yield) as a white solid.

LCMS (ESI): m/z 994.7 [M+H]⁺

### Step 2: N-(6-(1,1-dioxido-4-oxo-1,2,5-thiadiazolidin-2-yl)-5-fluoro-7-hydroxynaphthalen-2-yl)-6-(1-(2,6-dioxopiperidin-3-yl)-3-methyl-2-oxo-2,3-dihydro-1H-benzo[d]imidazol-5-yl)-1-methyl-1H-indole-3-carboxamide (Degrader 82)

To a solution of N-(7-(benzyloxy)-6-(1,1-dioxido-4-oxo-1,2,5-thiadiazolidin-2-yl)-5-fluoronaphthalen-2-yl)-6-(1-(2,6-bis(benzyloxy)pyridin-3-yl)-3-methyl-2-oxo-2,3-dihydro-1H-benzo[d]imidazol-5-yl)-1-methyl-1H-indole-3-carboxamide (3, 30 mg, 30.55 μmol, 1 eq) in DMF (2 mL) was added Pd/C (30 mg, 10% purity) and Pd(OH)₂/C (30 mg, 10% purity) under N₂ atmosphere. The suspension was degassed and purged with H₂ for 3 times. The mixture was stirred under H₂ (15 Psi) at 25° C. for 12 h. The reaction mixture was filtered and concentrated under reduced pressure. The residue was purified by prep-HPLC (flow: 25 mL/min; gradient: from 33-63% water (0.1% TFA)-ACN over 7 min; column: 3_Phenomenex Luna C18 75*30 mm*3 um) to afford N-(6-(1,1-dioxido-4-oxo-1,2,5-thiadiazolidin-2-yl)-5-fluoro-7-hydroxynaphthalen-2-yl)-6-(1-(2,6-dioxopiperidin-3-yl)-3-methyl-2-oxo-2,3-dihydro-1H-benzo[d]imidazol-5-yl)-1-methyl-1H-indole-3-carboxamide (Degrader 82, 4.5 mg, 5.09 μmol, 17% yield) as a yellow solid.

LCMS (ESI): m/z 726.4 [M+H]⁺

¹H NMR (400 MHz, DMSO-d₆) δ=11.13 (s, 1H), 10.03-9.76 (m, 2H), 8.37-8.20 (m, 3H), 7.92-7.82 (m, 2H), 7.69-7.58 (m, 3H), 7.49 (br d, J=8.0 Hz, 1H), 7.23 (br d, J=8.4 Hz, 1H), 6.99 (s, 1H), 5.45-5.40 (m, 1H), 4.14 (s, 2H), 3.98 (s, 3H), 3.45 (s, 4H), 2.96 (s, 1H), 2.81-2.65 (m, 2H), 2.14-2.03 (m, 1H), 1.52-1.39 (m, 1H), 1.28-1.02 (m, 1H)

### N-(6-(1,1-dioxido-4-oxo-1,2,5-thiadiazolidin-2-yl)-5-fluoro-7-hydroxynaphthalen-2-yl)-2-(5-(1-(2,6-dioxopiperidin-3-yl)-3-methyl-2-oxo-2,3-dihydro-1H-benzo[d]imidazol-5-yl)-6-fluoro-1H-indazol-1-yl)acetamide (Degrader 83)

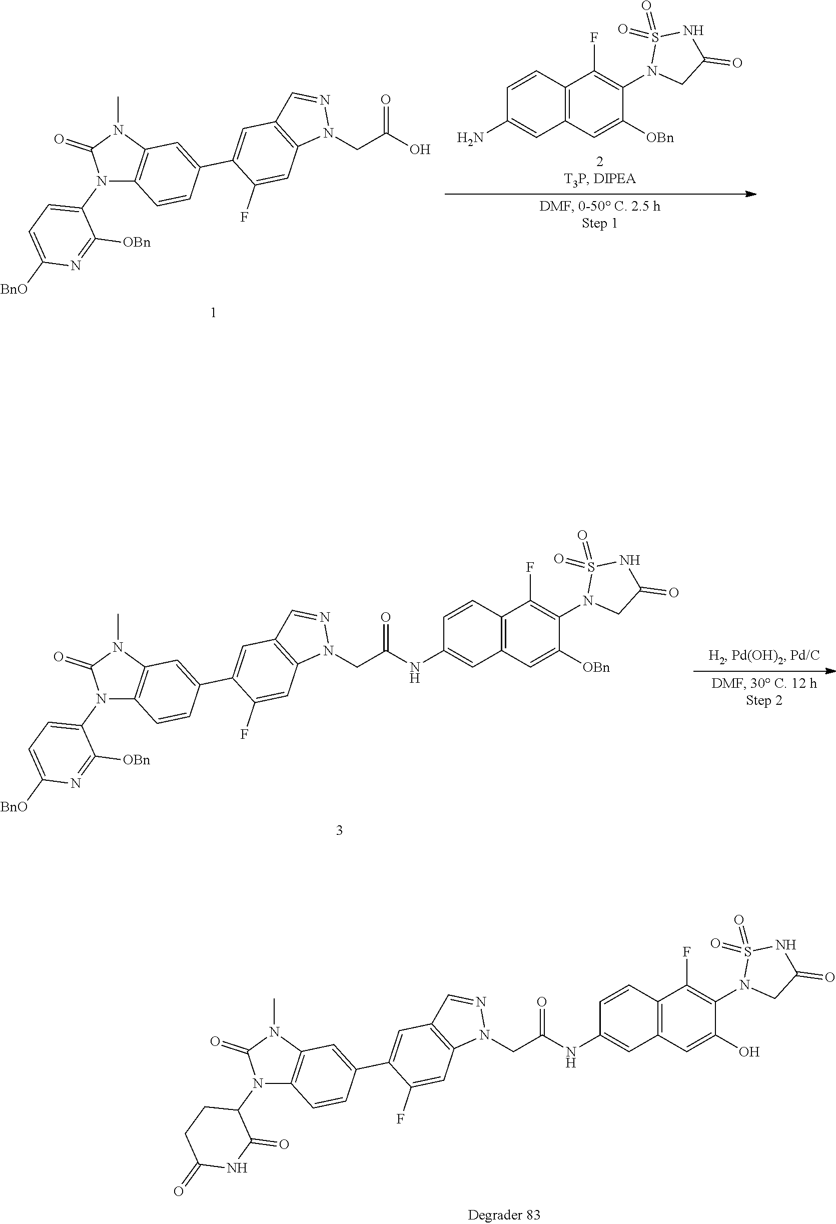 [SMILES: Cn1c(=O)n(-c2ccc(OCc3ccccc3)nc2OCc2ccccc2)c2ccc(-c3cc4cnn(CC(=O)O)c4cc3F)cc21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(-c3cc4cnn(CC(=O)Nc5ccc6c(F)c(N7CC(=O)NS7(=O)=O)c(OCc7ccccc7)cc6c5)c4cc3F)cc21 ; Cn1c(=O)n(C2CCC(=O)NC2=O)c2ccc(-c3cc4cnn(CC(=O)Nc5ccc6c(F)c(N7CC(=O)NS7(=O)=O)c(O)cc6c5)c4cc3F)cc21]

### Step 1: N-(7-(benzyloxy)-6-(1,1-dioxido-4-oxo-1,2,5-thiadiazolidin-2-yl)-5-fluoronaphthalen-2-yl)-2-(5-(1-(2,6-bis(benzyloxy)pyridin-3-yl)-3-methyl-2-oxo-2,3-dihydro-1H-benzo[d]imidazol-5-yl)-6-fluoro-1H-indazol-1-yl)acetamide (3)

To a solution of 2-[5-[1-(2,6-dibenzyloxy-3-pyridyl)-3-methyl-2-oxo-benzimidazol-5-yl]-6-fluoro-indazol-1-yl]acetic acid (1, 200 mg, 317.64 μmol) and DIPEA (205.27 mg, 1.59 mmol, 276.64 μL) in DMF (3 mL) was added T₃P (606.41 mg, 952.93 μmol, 50% purity) at 0° C. The mixture was stirred at 0° C. for 30 min, then 5-(6-amino-3-(benzyloxy)-1-fluoronaphthalen-2-yl)-1,2,5-thiadiazolidin-3-one 1,1-dioxide (2, 127.51 mg, 317.64 μmol) was added. The mixture was stirred at 50° C. for 2 h. The resulting mixture was filtered, and the filtrate was concentrated under reduced pressure. The residue was purified by prep-HPLC (flow: 25 mL/min; gradient: from 32-62% water (0.05% NH₃H₂O) in MeCN over 10 min; column: Waters Xbridge 150*25 mm*5 um).

The desired fraction was concentrated under reduced pressure to remove ACN. The aqueous phase was lyophilized to give N-(7-(benzyloxy)-6-(1,1-dioxido-4-oxo-1,2,5-thiadiazolidin-2-yl)-5-fluoronaphthalen-2-yl)-2-(5-(1-(2,6-bis(benzyloxy)pyridin-3-yl)-3-methyl-2-oxo-2,3-dihydro-1H-benzo[d]imidazol-5-yl)-6-fluoro-1H-indazol-1-yl)acetamide (3, 300 mg, 296.14 μmol, 93% yield) as a white solid.

LCMS (ESI): m/z 1013.2 [M+H]⁺

### Step 2: N-(6-(1,1-dioxido-4-oxo-1,2,5-thiadiazolidin-2-yl)-5-fluoro-7-hydroxynaphthalen-2-yl)-2-(5-(1-(2,6-dioxopiperidin-3-yl)-3-methyl-2-oxo-2,3-dihydro-1H-benzo[d]imidazol-5-yl)-6-fluoro-1H-indazol-1-yl)acetamide (Degrader 83)

To a solution of N-[7-benzyloxy-5-fluoro-6-(1,1,4-trioxo-1,2,5-thiadiazolidin-2-yl)-2-naphthyl]-2-[5-[1-(2,6-dibenzyloxy-3-pyridyl)-3-methyl-2-oxo-benzimidazol-5-yl]-6-fluoro-indazol-1-yl]acetamide (3, 0.1 g, 98.71 μmol) in DMF (3 mL) were added Pd/C (66.67 mg, 54.89 μmol, 10% purity) and Pd(OH)₂/C (50 mg, 98.71 μmol, 20% purity) under N₂. The mixture was stirred at 30° C. for 12 h under H₂ (15 psi). The resulting mixture was filtered through Celite, and the filtrate was concentrated under reduced pressure. The crude product was re-purified by prep-HPLC (flow: 25 mL/min; gradient: from 32-62% water (0.1% TFA) in MeCN over 7 min; column: Phenomenex Luna C18 75×30 mm×3 μm) and lyophilized to give N-(6-(1,1-dioxido-4-oxo-1,2,5-thiadiazolidin-2-yl)-5-fluoro-7-hydroxynaphthalen-2-yl)-2-(5-(1-(2,6-dioxopiperidin-3-yl)-3-methyl-2-oxo-2,3-dihydro-1H-benzo[d]imidazol-5-yl)-6-fluoro-1H-indazol-1-yl)acetamide (Degrader 83, 36.83 mg, 42.89 μmol, 43% yield) as a white solid.

LCMS (ESI): m/z 745.1 [M+H]⁺

1H NMR (400 MHz, DMSO-d6) δ=11.10 (s, 1H), 10.68 (s, 1H), 10.30 (s, 1H), 8.19 (s, 1H), 8.16 (s, 1H), 7.88 (dd, J=8.4, 13.2 Hz, 2H), 7.74 (d, J=11.0 Hz, 1H), 7.46 (dd, J=1.8, 9.2 Hz, 1H), 7.38 (s, 1H), 7.30 (s, 2H), 6.95 (s, 1H), 5.50-5.37 (m, 3H), 4.33 (s, 2H), 3.40 (s, 3H), 2.96-2.72 (m, 2H), 2.64 (br d, J=15.6 Hz, 1H), 2.06 (td, J=3.3, 6.7 Hz, 1H).

### N-(6-(1,1-dioxido-4-oxo-1,2,5-thiadiazolidin-2-yl)-5-fluoro-7-hydroxynaphthalen-2-yl)-2-(7-(1-(2,6-dioxopiperidin-3-yl)-3-methyl-2-oxo-2,3-dihydro-1H-benzo[d]imidazol-5-yl)-2,7-diazaspiro[3.5]nonan-2-yl)acetamide (Degrader 84)

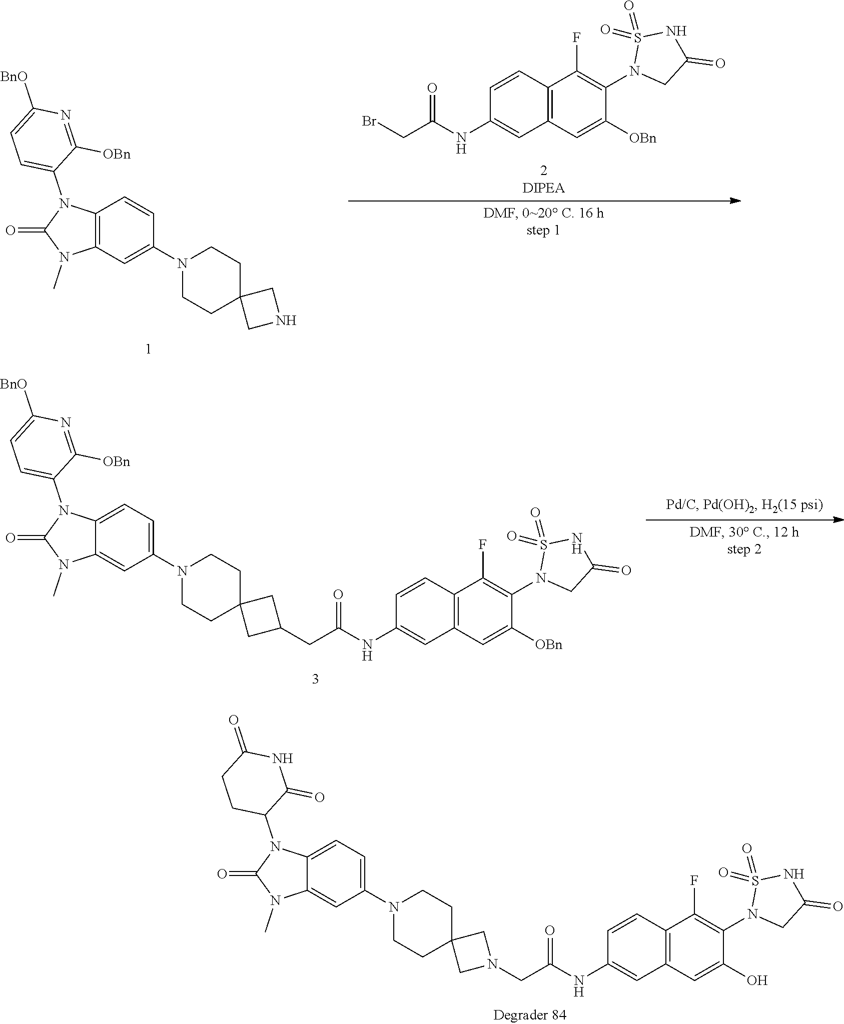 [SMILES: Cn1c(=O)n(-c2ccc(OCc3ccccc3)nc2OCc2ccccc2)c2ccc(N3CCC4(CC3)CNC4)cc21 ; O=C(CBr)Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(N3CCC4(CC3)CC(CC(=O)Nc3ccc5c(F)c(N6CC(=O)NS6(=O)=O)c(OCc6ccccc6)cc5c3)C4)cc21 ; Cn1c(=O)n(C2CCC(=O)NC2=O)c2ccc(N3CCC4(CC3)CN(CC(=O)Nc3ccc5c(F)c(N6CC(=O)NS6(=O)=O)c(O)cc5c3)C4)cc21]

### Step 1: N-(7-(benzyloxy)-6-(1,1-dioxido-4-oxo-1,2,5-thiadiazolidin-2-yl)-5-fluoronaphthalen-2-yl)-2-(7-(1-(2,6-bis(benzyloxy)pyridin-3-yl)-3-methyl-2-oxo-2,3-dihydro-1H-benzo[d]imidazol-5-yl)-2,7-diazaspiro[3.5]nonan-2-yl)acetamide (3)

To a mixture of 5-(2,7-diazaspiro[3.5]nonan-7-yl)-1-(2,6-dibenzyloxy-3-pyridyl)-3-methyl-benzimidazol-2-one (1, 160 mg, 263.29 μmol, formic acid salt) and N-(7-(benzyloxy)-6-(1,1-dioxido-4-oxo-1,2,5-thiadiazolidin-2-yl)-5-fluoronaphthalen-2-yl)-2-bromoacetamide (2, 152 mg, 291.00 μmol) in DMF (2 mL) was added DIPEA (170.14 mg, 1.32 mmol, 229.30 uL). The mixture was stirred at 15° C. for 16 h. The reaction mixture was filtered and purified by reversed phase HPLC (NH₃H₂O) and then lyophilization. The N-(7-(benzyloxy)-6-(1,1-dioxido-4-oxo-1,2,5-thiadiazolidin-2-yl)-5-fluoronaphthalen-2-yl)-2-(7-(1-(2,6-bis(benzyloxy)pyridin-3-yl)-3-methyl-2-oxo-2,3-dihydro-1H-benzo[d]imidazol-5-yl)-2,7-diazaspiro[3.5]nonan-2-yl)acetamide (3, 150 mg, 134.58 μmol, 51% yield) as off-white solid.

LCMS (ESI): m/z 1003.2 [M+H]⁺

### Step 2: N-(6-(1,1-dioxido-4-oxo-1,2,5-thiadiazolidin-2-yl)-5-fluoro-7-hydroxynaphthalen-2-yl)-2-(7-(1-(2,6-dioxopiperidin-3-yl)-3-methyl-2-oxo-2,3-dihydro-1H-benzo[d]imidazol-5-yl)-2,7-diazaspiro[3.5]nonan-2-yl)acetamide (Degrader 84)

To a solution of N-(7-(benzyloxy)-6-(1,1-dioxido-4-oxo-1,2,5-thiadiazolidin-2-yl)-5-fluoronaphthalen-2-yl)-2-(7-(1-(2,6-bis(benzyloxy)pyridin-3-yl)-3-methyl-2-oxo-2,3-dihydro-1H-benzo[d]imidazol-5-yl)-2,7-diazaspiro[3.5]nonan-2-yl)acetamide (3, 80 mg, 79.75 μmol) in DMF (4 mL) was added Pd/C (80 mg, 19.94 μmol, 10% purity) and Pd(OH)₂ (80 mg, 19.94 μmol, 10% purity) under N₂ atmosphere. The suspension was degassed and purged with H₂ 3 times. The mixture was stirred under H₂ (15 Psi) at 25° C. for 12 h. The reaction mixture was filtered and concentrated under vacuum. The residue was purified by prep-HPLC (flow: 25 mL/min; gradient: from 8-38% MeCN in water (0.1% TFA) over 12 min; column: Phenomenex Synergi C18 150×25 mm×10 um) to afford N-(6-(1,1-dioxido-4-oxo-1,2,5-thiadiazolidin-2-yl)-5-fluoro-7-hydroxynaphthalen-2-yl)-2-(7-(1-(2,6-dioxopiperidin-3-yl)-3-methyl-2-oxo-2,3-dihydro-1H-benzo[d]imidazol-5-yl)-2,7-diazaspiro[3.5]nonan-2-yl)acetamide (Degrader 84, 58.14 mg, 62.33 μmol, 78% yield, TFA salt) as a pink solid.

LCMS (ESI): m/z 735.1 [M+H]⁺

¹H NMR (400 MHz, DMSO-d₆) δ 11.07 (s, 1H), 10.72 (s, 1H), 10.52 (br d, J=4.8 Hz, 1H), 10.18-9.93 (m, 1H), 8.14-8.04 (m, 1H), 7.94-7.87 (m, 1H), 7.49-7.40 (m, 1H), 7.07-6.90 (m, 3H), 6.82-6.69 (m, 1H), 5.31 (m, 1H), 4.39 (br d, J=4.4 Hz, 2H), 4.22 (s, 2H), 4.09 (m, 4H), 3.32 (s, 3H), 3.26-3.07 (m, 4H), 2.95-2.84 (m, 1H), 2.70-2.61 (m, 2H), 2.13-1.89 (m, 5H).

### 2-[4-[1-(2,6-dioxo-3-piperidyl)-3-methyl-indazol-5-yl]phenyl]-N-[5-fluoro-7-hydroxy-6-(1,1,4-trioxo-1,2,5-thiadiazolidin-2-yl)-2-naphthyl]acetamide (Degrader 85)

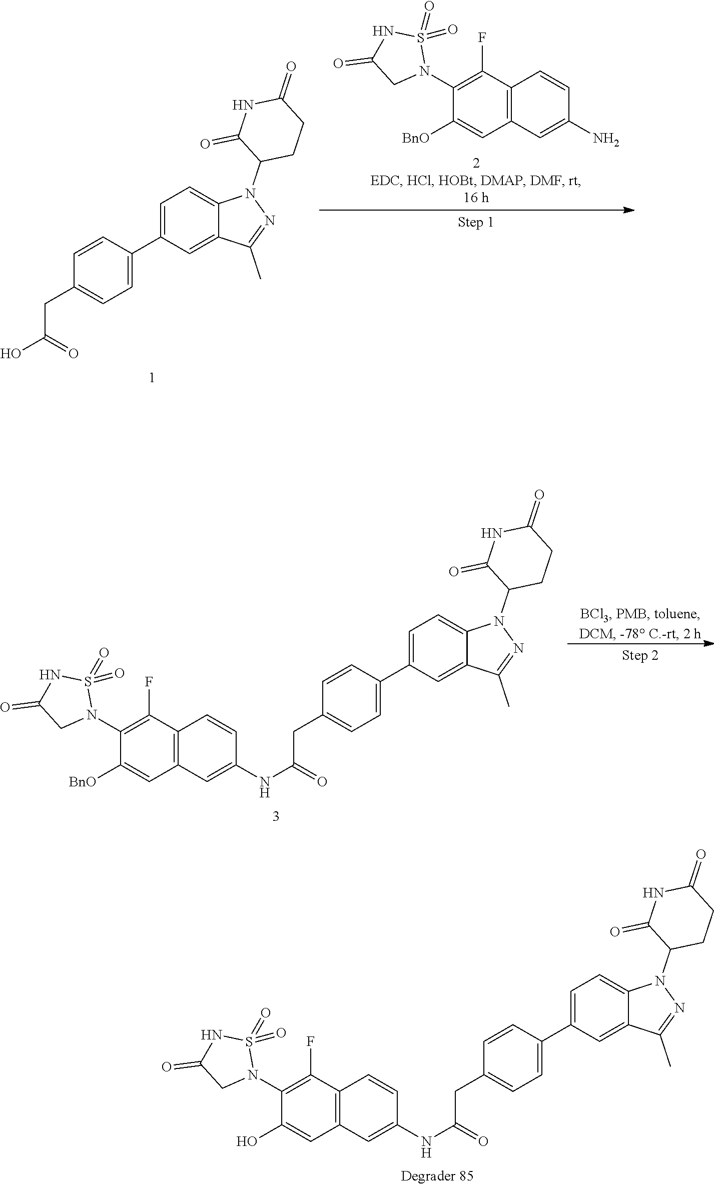 [SMILES: Cc1nn(C2CCC(=O)NC2=O)c2ccc(-c3ccc(CC(=O)O)cc3)cc12 ; Nc1ccc2c(F)c(N3CC(=O)NS3(=O)=O)c(OCc3ccccc3)cc2c1 ; Cc1nn(C2CCC(=O)NC2=O)c2ccc(-c3ccc(CC(=O)Nc4ccc5c(F)c(N6CC(=O)NS6(=O)=O)c(OCc6ccccc6)cc5c4)cc3)cc12 ; Cc1nn(C2CCC(=O)NC2=O)c2ccc(-c3ccc(CC(=O)Nc4ccc5c(F)c(N6CC(=O)NS6(=O)=O)c(O)cc5c4)cc3)cc12]

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-indazol-5-yl]phenyl]acetamide (5)

To a 10 mL single-neck round-bottom flask containing a solution of 2-[4-[1-(2,6-dioxo-3-piperidyl)-3-methyl-indazol-5-yl]phenyl]acetic acid (1, 140 mg, 304.19 μmol) and 5-(6-amino-3-benzyloxy-1-fluoro-2-naphthyl)-1,1-dioxo-1,2,5-thiadiazolidin-3-one (2, 161.64 mg, 304.19 μmol, TFA salt) in anhydrous DMF (0.7 mL) were added EDC·HCl (174.94 mg, 912.58 μmol), HOBT (102.76 mg, 760.48 μmol) and DMAP (260.14 mg, 2.13 mmol) at room temperature. The resulting mixture was stirred at room temperature. After 16 h, the solvent was removed under reduced pressure and the residue was treated with aqueous 1.5N HCl (30 mL) was added. The separated off white solid was filtered and dried under reduced pressure to get crude N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-indazol-5-yl]phenyl]acetamide (3, 234 mg, 110.98 μmol, 36% yield, HCl salt) as brown solid.

LCMS (ES+): m/z 761.0 [M+H]⁺

### Step 2: 2-[4-[1-(2,6-dioxo-3-piperidyl)-3-methyl-indazol-5-yl]phenyl]-N-[5-fluoro-7-hydroxy-6-(1,1,4-trioxo-1,2,5-thiadiazolidin-2-yl)-2-naphthyl]acetamide (Degrader 85)

Into a 50 mL single neck round-bottom flask containing a well-stirred solution of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-indazol-5-yl]phenyl]acetamide (3, 230 mg, 109.08 μmol, HCl salt, crude), pentamethylbenzene (16.17 mg, 109.08 μmol) in anhydrous toluene (3 mL) and DCM (3 mL) was added a 1.0 M solution of boron trichloride in methylene chloride (5.8 mmol, 5.8 mL) at −78° C. The reaction mixture was stirred at room temperature for 2 h. Then, the reaction mixture was quenched with 5% MeOH in DCM (3 mL) at −78° C. and concentrated under reduced pressure. The residue was triturated with MTBE (50 mL), filtered and dried. The crude compound was purified by reverse phase column chromatography [Silicycle C18 column; Mobile phase A: 0.1% formic acid in water; Mobile phase B: MeCN] to get 2-[4-[1-(2,6-dioxo-3-piperidyl)-3-methyl-indazol-5-yl]phenyl]-N-[5-fluoro-7-hydroxy-6-(1,1,4-trioxo-1,2,5-thiadiazolidin-2-yl)-2-naphthyl]acetamide (Degrader 85, 26 mg, 37.40 μmol, 34% yield) as off white solid.

LCMS (ES+): m/z 671.0 [M+H]⁺

¹H NMR (400 MHz, DMSO-d6): δ 11.08 (s, 1H), 10.44 (s, 1H), 10.08 (s, 1H), 8.18 (s, 1H), 8.00 (s, 1H), 7.86 (d, J=8.80 Hz, 1H), 7.74-7.71 (m, 3H), 7.63 (d, J=8.80 Hz, 1H), 7.48-7.46 (m, 3H), 6.95 (s, 1H), 5.81-5.76 (m, 1H), 4.24 (s, 2H), 3.76 (s, 2H), 2.89-2.74 (m, 4H), 2.54 (s, 3H), 2.34-2.28 (m, 1H).

### 1-[4-[9-(2,6-dioxo-3-piperidyl)pyrido[2,3-b]indol-6-yl]butyl]-3-[5-fluoro-7-hydroxy-6-(1,1,4-trioxo-1,2,5-thiadiazolidin-2-yl)-2-naphthyl]urea (Degrader 86)

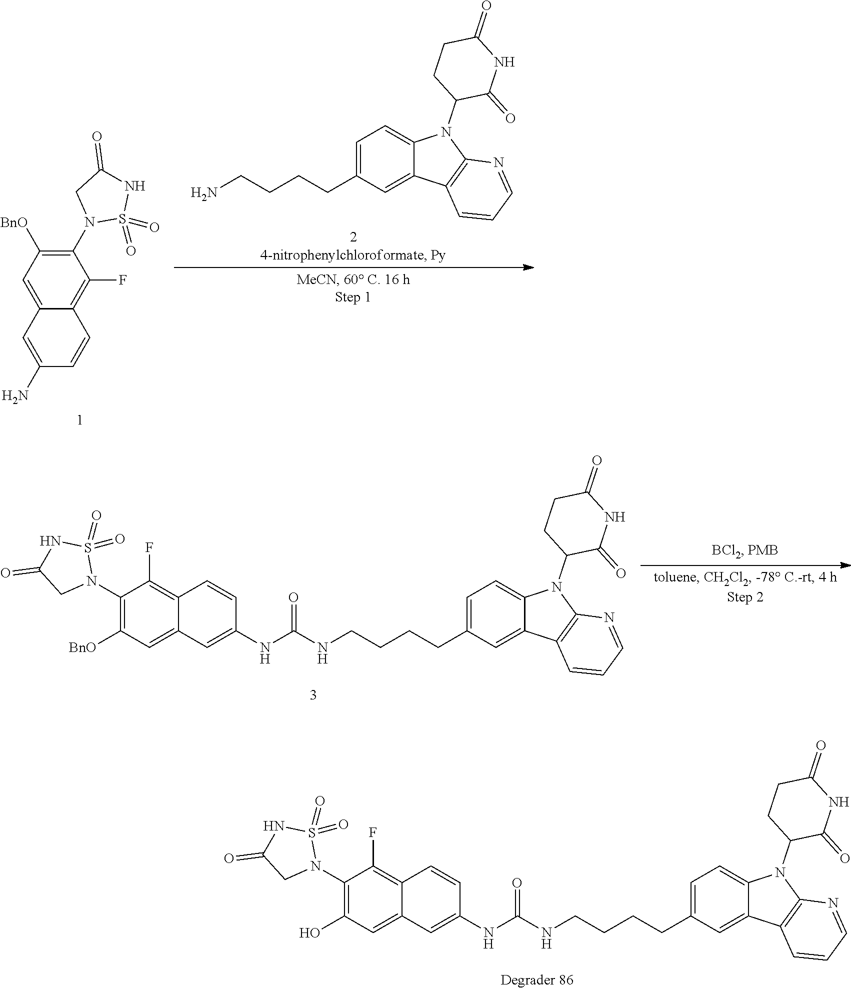 [SMILES: Nc1ccc2c(F)c(N3CC(=O)NS3(=O)=O)c(OCc3ccccc3)cc2c1 ; NCCCCc1ccc2c(c1)c1cccnc1n2C1CCC(=O)NC1=O ; O=C1CCC(n2c3ccc(CCCCNC(=O)Nc4ccc5c(F)c(N6CC(=O)NS6(=O)=O)c(OCc6ccccc6)cc5c4)cc3c3cccnc32)C(=O)N1 ; O=C1CCC(n2c3ccc(CCCCNC(=O)Nc4ccc5c(F)c(N6CC(=O)NS6(=O)=O)c(O)cc5c4)cc3c3cccnc32)C(=O)N1]

### Step 1: 1-[7-benzyloxy-5-fluoro-6-(1,1,4-trioxo-1,2,5-thiadiazolidin-2-yl)-2-naphthyl]-3-[4-[9-(2,6-dioxo-3-piperidyl)pyrido[2,3-b]indol-6-yl]butyl]urea (3)

Into a 25 mL single-neck round-bottom flask containing a well-stirred solution of 5-(6-amino-3-benzyloxy-1-fluoro-2-naphthyl)-1,1-dioxo-1,2,5-thiadiazolidin-3-one (1, 40 mg, 76.83 μmol, TFA salt) in dry acetonitrile (2 mL) was added 4-nitrophenyl chloroformate (23.23 mg, 115.24 μmol) followed by pyridine (30.39 mg, 384.14 μmol, 31.07 μL) and the resulting mixture was stirred for 2 h at room temperature. Afterwards, 3-[6-(4-aminobutyl)pyrido[2,3-b]indol-9-yl]piperidine-2,6-dione (2, 36.04 mg, 76.83 μmol, TFA salt) was added and stirring was continued at 60° C. for 16 h. Subsequently, the solvent was evaporated under reduced pressure to get residue which was diluted with ice cold water to precipitate a solid. Filtration and drying afforded 1-[7-benzyloxy-5-fluoro-6-(1,1,4-trioxo-1,2,5-thiadiazolidin-2-yl)-2-naphthyl]-3-[4-[9-(2,6-dioxo-3-piperidyl)pyrido[2,3-b]indol-6-yl]butyl]urea (3, 60 mg, 28.67 μmol, 37% yield) as a brown color solid.

LCMS (ES−): m/z 776.0 [M−H]⁻

### Step 2:1-[4-[9-(2,6-dioxo-3-piperidyl)pyrido[2,3-b]indol-6-yl]butyl]-3-[5-fluoro-7-hydroxy-6-(1,1,4-trioxo-1,2,5-thiadiazolidin-2-yl)-2-naphthyl]urea (Degrader 86)

Into a 25 mL single-neck round-bottom flask containing a well-stirred solution of 1-[7-benzyloxy-5-fluoro-6-(1,1,4-trioxo-1,2,5-thiadiazolidin-2-yl)-2-naphthyl]-3-[4-[9-(2,6-dioxo-3-piperidyl)pyrido[2,3-b]indol-6-yl]butyl]urea (3, 60 mg, 28.67 μmol) in dry toluene (1 mL) and dry DCM (1 mL) was added pentamethylbenzene (21.25 mg, 143.36 μmol) under nitrogen atmosphere at room temperature. The reaction mixture was then cooled to −78° C. and BCl₃ solution (1.0 M solution in DCM, 573.45 μmol, 573.00 μL) was slowly added to the reaction mixture. Subsequently, the mixture was stirred at room temperature for 4 h. The reaction mixture was quenched with 5% MeOH in DCM (1 mL) at −78° C. The mixture was concentrated under reduced pressure and purified by reverse-phase preparative HPLC [Purification method: Column: X-BRIDGE C8 (150×19) mm, 5 microns; Mobile phase A: 0.1% ammonium acetate in water and Mobile Phase B: MeCN] to get 1-[4-[9-(2,6-dioxo-3-piperidyl)pyrido[2,3-b]indol-6-yl]butyl]-3-[5-fluoro-7-hydroxy-6-(1,1,4-trioxo-1,2,5-thiadiazolidin-2-yl)-2-naphthyl]urea (Degrader 86, 7.5 mg, 10.23 μmol, 36% yield) as a brown gummy solid.

LCMS (ES+): m/z 688.0 [M+H]⁺

¹H-NMR (400 MHz, DMSO-d6): δ 11.12 (s, 1H), 8.84 (s, 1H), 8.51 (d, J=6.00 Hz, 1H), 8.40 (dd, J=1.20, 4.80 Hz, 1H), 8.08 (s, 1H), 7.88 (s, 1H), 7.72 (d, J=9.20 Hz, 1H), 7.53 (d, J=9.20 Hz, 1H), 7.39 (d, J=7.60 Hz, 1H), 7.25-7.21 (m, 2H), 6.85 (s, 1H), 6.52-6.42 (m, 1H), 6.05-5.95 (m, 1H), 4.06 (s, 2H), 3.10-2.90 (m, 2H), 2.80 (t, J=7.20 Hz, 2H), 2.18-2.05 (m, 2H), 1.76-1.67 (m, 4H), 1.58-1.49 (m, 2H).

### N-(6-(1,1-dioxido-4-oxo-1,2,5-thiadiazolidin-2-yl)-5-fluoro-7-hydroxynaphthalen-2-yl)-2-(4-((1-(2,6-dioxopiperidin-3-yl)-3-methyl-2-oxo-2,3-dihydro-1H-benzo[d]imidazol-5-yl)(methyl)amino)-3-methylphenyl)acetamide (Degrader 87)

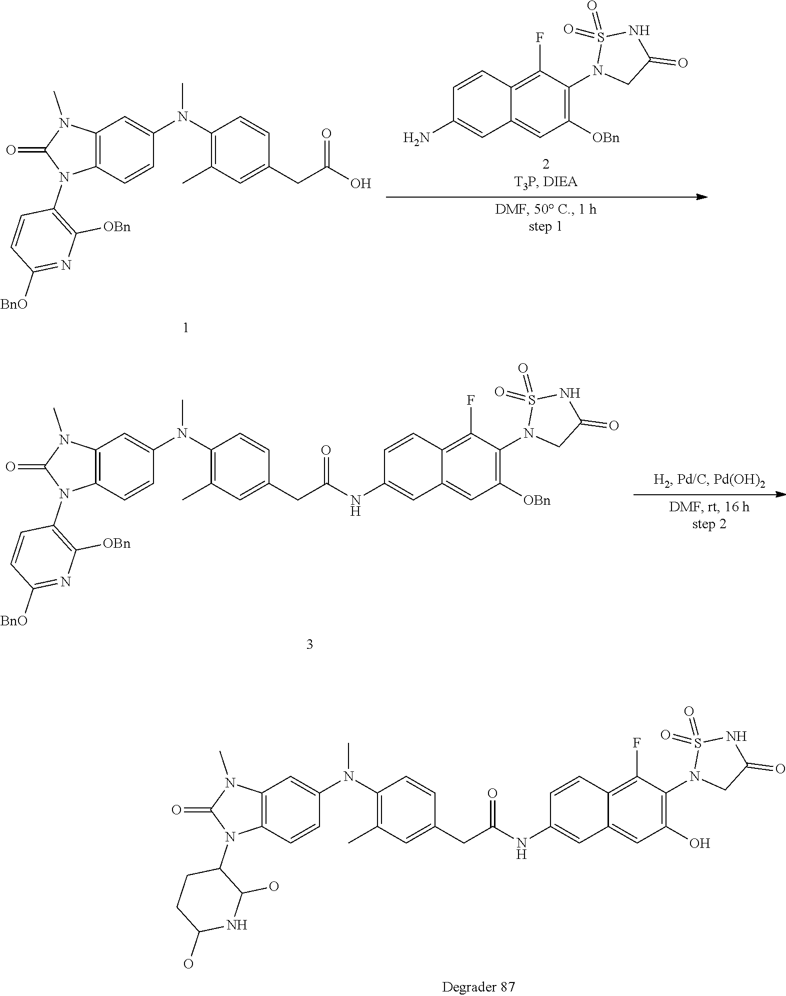 [SMILES: Cc1cc(CC(=O)O)ccc1N(C)c1ccc2c(c1)n(C)c(=O)n2-c1ccc(OCc2ccccc2)nc1OCc1ccccc1 ; Nc1ccc2c(F)c(N3CC(=O)NS3(=O)=O)c(OCc3ccccc3)cc2c1 ; Cc1cc(CC(=O)Nc2ccc3c(F)c(N4CC(=O)NS4(=O)=O)c(OCc4ccccc4)cc3c2)ccc1N(C)c1ccc2c(c1)n(C)c(=O)n2-c1ccc(OCc2ccccc2)nc1OCc1ccccc1 ; Cc1cc(CC(=O)Nc2ccc3c(F)c(N4CC(=O)NS4(=O)=O)c(O)cc3c2)ccc1N(C)c1ccc2c(c1)n(C)c(=O)n2C1CCC(O)NC1O]

### Step 1: N-(7-(benzyloxy)-6-(1,1-dioxido-4-oxo-1,2,5-thiadiazolidin-2-yl)-5-fluoronaphthalen-2-yl)-2-(4-((1-(2,6-bis(benzyloxy)pyridin-3-yl)-3-methyl-2-oxo-2,3-dihydro-1H-benzo[d]imidazol-5-yl)(methyl)amino)-3-methylphenyl)acetamide (3)

To a solution of 2-[4-[[1-(2,6-dibenzyloxy-3-pyridyl)-3-methyl-2-oxo-benzimidazol-5-yl]-methyl-amino]-3-methyl-phenyl]acetic acid (1, 0.05 g, 81.34 μmol) and 5-(6-amino-3-benzyloxy-1-fluoro-2-naphthyl)-1,1-dioxo-1,2,5-thiadiazolidin-3-one (2, 32.65 mg, 81.34 μmol) in DMF (1 mL) were added T₃P (50 wt % in EtOAc) (155.29 mg, 244.03 μmol, 147.89 μL, 50% purity) and DIEA (41.16 mg, 406.71 μmol, 56.69 μL) at 0° C. The mixture was stirred at 50° C. for 1 h. The mixture was filtered to remove the insoluble. The filtrate was purified by prep-HPLC (flow: 25 mL/min; gradient: from 30-60% MeCN in water (0.05% ammonia hydroxide v/v) over 10 min; column: Waters Xbridge 150*25 mm*5 um). N-[7-benzyloxy-5-fluoro-6-(1,1,4-trioxo-1,2,5-thiadiazolidin-2-yl)-2-naphthyl]-2-[4-[[1-(2,6-dibenzyloxy-3-pyridyl)-3-methyl-2-oxo-benzimidazol-5-yl]-methyl-amino]-3-methyl-phenyl]acetamide (3, 50 mg, 49.60 μmol, 61% yield) as a yellow solid.

LCMS (ESI): m/z 997.3 [M]⁺

### Step 2: N-(6-(1,1-dioxido-4-oxo-1,2,5-thiadiazolidin-2-yl)-5-fluoro-7-hydroxynaphthalen-2-yl)-2-(4-((1-(2,6-dioxopiperidin-3-yl)-3-methyl-2-oxo-2,3-dihydro-1H-benzo[d]imidazol-5-yl)(methyl)amino)-3-methylphenyl)acetamide (Degrader 87)

To a solution of N-[7-benzyloxy-5-fluoro-6-(1,1,4-trioxo-1,2,5-thiadiazolidin-2-yl)-2-naphthyl]-2-[4-[[1-(2,6-dibenzyloxy-3-pyridyl)-3-methyl-2-oxo-benzimidazol-5-yl]-methyl-amino]-3-methyl-phenyl]acetamide (3, 0.06 g, 60.12 μmol) in DMF were added Pd/C (60 mg, 49.40 μmol, 10% purity) and Pd(OH)₂ (60 mg, 60.12 μmol, 20% purity). The mixture was stirred at 30° C. for 16 hr under H₂ (15 psi). The mixture was filtered and washed with DMF (1 mL×3). The filtrate was purified by prep-HPLC (flow: 25 mL/min; gradient: from 40-70% MeCN in water (0.1% TFA) over 10 min; column: Phenomenex Synergi C18 150×25 mm×10 um). N-(6-(1,1-dioxido-4-oxo-1,2,5-thiadiazolidin-2-yl)-5-fluoro-7-hydroxynaphthalen-2-yl)-2-(4-((1-(2,6-dioxopiperidin-3-yl)-3-methyl-2-oxo-2,3-dihydro-1H-benzo[d]imidazol-5-yl)(methyl)amino)-3-methylphenyl)acetamide (Degrader 87, 43.81 mg, 51.92 μmol, 86% yield, TFA salt) as a pink solid.

¹H NMR (400 MHz, DMSO) δ 11.03 (s, 1H), 10.41 (s, 1H), 10.24 (s, 1H), 8.19 (s, 1H), 7.86 (d, J=9.0 Hz, 1H), 7.46 (dd, J=9.1, 2.0 Hz, 1H), 7.32-7.27 (m, 1H), 7.28-7.22 (m, 1H), 7.08 (d, J=8.0 Hz, 1H), 6.96 (s, 1H), 6.84 (d, J=8.6 Hz, 1H), 6.48 (d, J=2.3 Hz, 1H), 6.08 (dd, J=8.6, 2.3 Hz, 1H), 5.25 (dd, J=12.9, 5.3 Hz, 1H), 4.32 (s, 2H), 3.69 (s, 2H), 3.25 (s, 3H), 3.17 (s, 3H), 2.93-2.82 (m, 1H), 2.71-2.58 (m, 2H), 2.09 (s, 3H), 2.01-1.89 (m, 2H).

### N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)-1H-pyrazol-3-yl)acetamide (Degrader 88)

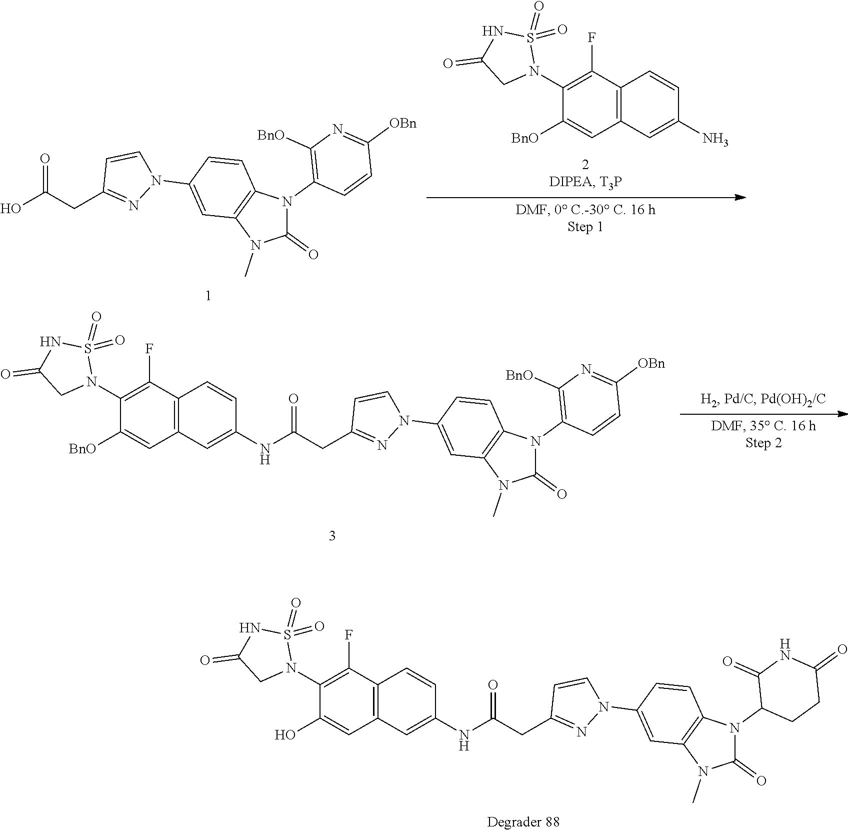 [SMILES: Cn1c(=O)n(-c2ccc(OCc3ccccc3)nc2OCc2ccccc2)c2ccc(-n3ccc(CC(=O)O)n3)cc21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(-n3ccc(CC(=O)Nc4ccc5c(F)c(N6CC(=O)NS6(=O)=O)c(OCc6ccccc6)cc5c4)n3)cc21 ; Cn1c(=O)n(C2CCC(=O)NC2=O)c2ccc(-n3ccc(CC(=O)Nc4ccc5c(F)c(N6CC(=O)NS6(=O)=O)c(O)cc5c4)n3)cc21]

### Step 1 N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-methyl-2-oxo-2,3-dihydro-1H-benzo[d]imidazol-5-yl)-1H-pyrazol-3-yl)acetamide (3)

A solution of 2-[1-[1-(2,6-dibenzyloxy-3-pyridyl)-3-methyl-2-oxo-benzimidazol-5-yl]pyrazol-3-yl]acetic acid (1, 280 mg, 498.59 μmol) and 5-(6-amino-3-(benzyloxy)-1-fluoronaphthalen-2-yl)-1,2,5-thiadiazolidin-3-one 1,1-dioxide (2, 200.14 mg, 498.59 μmol) was added into the mixture of N-ethyl-N-isopropyl-propan-2-amine (451.07 mg, 3.49 mmol, 607.91 μL) in DMF (3 mL). Then T₃P (1.85 mg, 1.50 mmol, 50% purity) was added into the mixture at 0° C. The reaction mixture was stirred at 30° C. for 16 h. The reaction mixture was diluted with DMSO (1 mL) and purified by reversed phased HPLC (NH₃·H₂O). The target peak was concentrated to afford N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-methyl-2-oxo-2,3-dihydro-1H-benzo[d]imidazol-5-yl)-1H-pyrazol-3-yl)acetamide (3, 100 mg, 103.71 μmol, 21% yield) as white solid

LCMS (ESI): m/z 945.1 [M+H]⁺

### Step 2: N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)-1H-pyrazol-3-yl)acetamide (Degrader 88)

To a solution of N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-methyl-2-oxo-2,3-dihydro-1H-benzo[d]imidazol-5-yl)-1H-pyrazol-3-yl)acetamide (3, 100 mg, 105.82 μmol) in DMF (2 mL) was added wet Pd/C (50.00 mg, 10% purity) and wet Pd(OH)₂ (50.00 mg, 50% purity). The reaction was stirred at 35° C. for 16 hr under H₂ (15 psi) atmosphere. The reaction mixture was filtered to remove black solid. The filtrate was purified by prep-HPLC (Column: 3_Phenomenex Luna C18 75*30 mm*3 um; water (0.1% TFA)-ACN, B: 22%-52%; Gradient Time: 7 min, FlowRate (ml/min): 25). The target peak was concentrated and lyophilizated to afford N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)-1H-pyrazol-3-yl)acetamide (Degrader 88, 35.55 mg, 44.21 μmol, 42% yield, TFA salt) as white solid.

LCMS (ESI): m/z 677.2 [M+H]⁺

¹H NMR (400 MHz, DMSO-d6) δ=11.12 (s, 1H), 10.75-10.31 (m, 2H), 8.40 (d, J=2.4 Hz, 1H), 8.21 (s, 1H), 7.88 (d, J=9.2 Hz, 1H), 7.65 (d, J=2.0 Hz, 1H), 7.50-7.47 (m, 2H), 7.22 (d, J=8.8 Hz, 1H), 6.97 (s, 1H), 6.51 (d, J=2.4 Hz, 1H), 5.42-5.38 (m, 1H), 4.46 (s, 2H), 3.82 (s, 2H), 3.40 (s, 3H), 2.96-2.86 (m, 1H), 2.79-2.71 (m, 1H), 2.66-2.61 (m, 1H), 2.09-2.02 (m, 1H).

### 1-(6-(1,1-dioxido-4-oxo-1,2,5-thiadiazolidin-2-yl)-5-fluoro-7-hydroxynaphthalen-2-yl)-3-((1-(1-(2,6-dioxopiperidin-3-yl)-3-methyl-2-oxo-2,3-dihydro-1H-benzo[d]imidazol-5-yl)piperidin-4-yl)methyl)urea (Degrader 89)

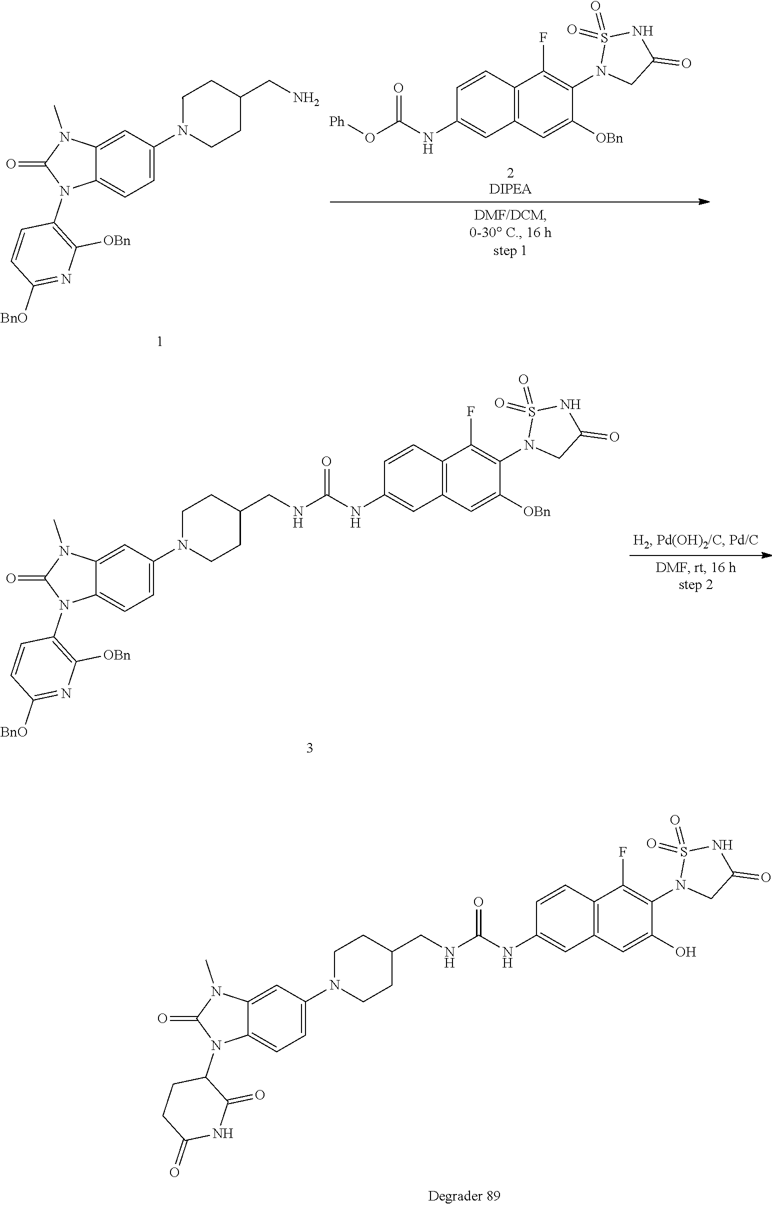 [SMILES: Cn1c(=O)n(-c2ccc(OCc3ccccc3)nc2OCc2ccccc2)c2ccc(N3CCC(CN)CC3)cc21 ; O=C1CN(c2c(OCc3ccccc3)cc3cc(NC(=O)Oc4ccccc4)ccc3c2F)S(=O)(=O)N1 ; Cn1c(=O)n(-c2ccc(OCc3ccccc3)nc2OCc2ccccc2)c2ccc(N3CCC(CNC(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(N3CCC(CNC(=O)Nc4ccc5c(F)c(N6CC(=O)NS6(=O)=O)c(O)cc5c4)CC3)cc21]

### Step 1: 1-(7-(benzyloxy)-6-(1,1-dioxido-4-oxo-1,2,5-thiadiazolidin-2-yl)-5-fluoronaphthalen-2-yl)-3-((1-(1-(2,6-bis(benzyloxy)pyridin-3-yl)-3-methyl-2-oxo-2,3-dihydro-1H-benzo[d]imidazol-5-yl)piperidin-4-yl)methyl)urea (3)

To a solution of 5-(4-(aminomethyl)piperidin-1-yl)-1-(2,6-bis(benzyloxy)pyridin-3-yl)-3-methyl-1H-benzo[d]imidazol-2 (3H)-one (1, 400 mg, 554.12 μmol, TsOH salt) and N-ethyl-N-isopropyl-propan-2-amine (5, 358.08 mg, 2.77 mmol, 482.59 μL) in DCM (4 mL) and DMF (4 mL) was added phenyl (7-(benzyloxy)-6-(1,1-dioxido-4-oxo-1,2,5-thiadiazolidin-2-yl)-5-fluoronaphthalen-2-yl)carbamate (2, 317.88 mg, 609.53 μmol) at 0° C. The mixture was stirred at 30° C. for 16 h. The mixture was filtered and the filtrate was concentrated in vacuum to give a residue. The residue was purified by prep-HPLC (flow: 25 mL/min; gradient: from 28-58% MeCN in water (0.05% NH₄HCO₃) over 12 min; column: Waters Xbridge 150×25 mm×5 um) to afford 1-(7-(benzyloxy)-6-(1,1-dioxido-4-oxo-1,2,5-thiadiazolidin-2-yl)-5-fluoronaphthalen-2-yl)-3-((1-(1-(2,6-bis(benzyloxy)pyridin-3-yl)-3-methyl-2-oxo-2,3-dihydro-1H-benzo[d]imidazol-5-yl)piperidin-4-yl)methyl)urea (3, 170 mg, 165.29 μmol, 30% yield) as a yellow solid.

LCMS (ESI): m/z 977.6 [M+H]⁺

### Step 2: 1-(6-(1,1-dioxido-4-oxo-1,2,5-thiadiazolidin-2-yl)-5-fluoro-7-hydroxynaphthalen-2-yl)-3-((1-(1-(2,6-dioxopiperidin-3-yl)-3-methyl-2-oxo-2,3-dihydro-1H-benzo[d]imidazol-5-yl)piperidin-4-yl)methyl)urea (Degrader 89)

To a solution of 1-(7-(benzyloxy)-6-(1,1-dioxido-4-oxo-1,2,5-thiadiazolidin-2-yl)-5-fluoronaphthalen-2-yl)-3-((1-(1-(2,6-bis(benzyloxy)pyridin-3-yl)-3-methyl-2-oxo-2,3-dihydro-1H-benzo[d]imidazol-5-yl)piperidin-4-yl)methyl)urea (3, 70 mg, 71.64 μmol) in DMF (3 mL) was added Pd/C (87.01 mg, 71.64 μmol, 10% purity) and Pd(OH)₂/C (70 mg, 71.64 μmol, 10% purity) under N₂ atmosphere. The suspension was degassed and purged with H₂ 3 times. The mixture was stirred under H₂ (15 psi) at 30° C. for 16 h. The reaction mixture was filtered and concentrated in vacuum. The residue was purified by prep-HPLC (flow: 25 mL/min; gradient: from 32-52% MeCN in water (0.1% TFA) over 9 min; column: 3_Phenomenex Luna C18 75×30 mm×3 μm) to afford 1-(6-(1,1-dioxido-4-oxo-1,2,5-thiadiazolidin-2-yl)-5-fluoro-7-hydroxynaphthalen-2-yl)-3-((1-(1-(2,6-dioxopiperidin-3-yl)-3-methyl-2-oxo-2,3-dihydro-1H-benzo[d]imidazol-5-yl)piperidin-4-yl)methyl)urea (Degrader 89, 34.87 mg, 41.96 μmol, 59% yield, TFA salt) as a white solid.

LCMS (ESI): m/z 709.3 [M+H]⁺

¹H NMR (400 MHz, DMSO) δ 11.11 (s, 1H), 9.92 (s, 1H), 8.79 (s, 1H), 7.92-7.88 (m, 1H), 7.77 (d, J=9.0 Hz, 1H), 7.32-7.26 (m, 1H), 7.20-7.05 (m, 2H), 6.88 (s, 1H), 6.52-6.45 (m, 1H), 5.43-5.32 (m, 1H), 4.22 (s, 2H), 3.63 (d, J=11.4 Hz, 3H), 3.35 (s, 3H), 3.18-3.09 (m, 2H), 2.96-2.84 (m, 1H), 2.77-2.57 (m, 2H), 2.06-1.97 (m, 1H), 1.95-1.88 (m, 2H), 1.85-1.73 (m, 1H), 1.63-1.47 (m, 2H).

### N-(6-(1,1-dioxido-4-oxo-1,2,5-thiadiazolidin-2-yl)-5-fluoro-7-hydroxynaphthalen-2-yl)-2-((2S)-4-(1-(2,6-dioxopiperidin-3-yl)-3-methyl-2-oxo-2,3-dihydro-1H-benzo[d]imidazol-5-yl)-2-methylpiperazin-1-yl)acetamide (Degrader 90)

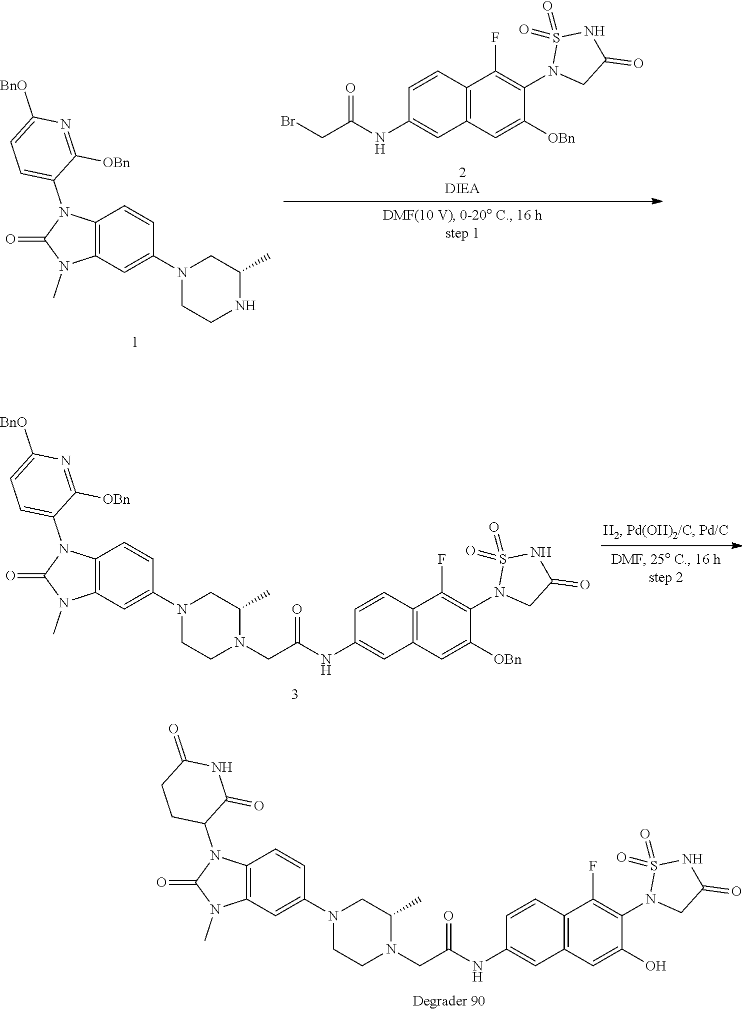 [SMILES: C[C@H]1CN(c2ccc3c(c2)n(C)c(=O)n3-c2ccc(OCc3ccccc3)nc2OCc2ccccc2)CCN1 ; O=C(CBr)Nc1ccc2c(F)c(N3CC(=O)NS3(=O)=O)c(OCc3ccccc3)cc2c1 ; C[C@H]1CN(c2ccc3c(c2)n(C)c(=O)n3-c2ccc(OCc3ccccc3)nc2OCc2ccccc2)CCN1CC(=O)Nc1ccc2c(F)c(N3CC(=O)NS3(=O)=O)c(OCc3ccccc3)cc2c1 ; C[C@H]1CN(c2ccc3c(c2)n(C)c(=O)n3C2CCC(=O)NC2=O)CCN1CC(=O)Nc1ccc2c(F)c(N3CC(=O)NS3(=O)=O)c(O)cc2c1]

### Step 1: (S)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-2-methylpiperazin-1-yl)acetamide (3)

To a mixture of 1-(2,6-dibenzyloxy-3-pyridyl)-3-methyl-5-[(3S)-3-methylpiperazin-1-yl]benzimidazol-2-one (1, 160 mg, 298.71 μmol) and N-(7-(benzyloxy)-6-(1,1-dioxido-4-oxo-1,2,5-thiadiazolidin-2-yl)-5-fluoronaphthalen-2-yl)-2-bromoacetamide (2, 172 mg, 329.29 μmol) in DMF (2 mL) was added DIPEA (193.03 mg, 1.49 mmol, 260.15 uL). The mixture was stirred at 15° C. for 16 h. The reaction mixture was filtered and purified by reversed phase HPLC (NH₃·H₂O) and then lyophilization to give the (S)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-2-methylpiperazin-1-yl)acetamide (3, 110 mg, 111.46 μmol, 37% yield) as off-white solid.

LCMS (ESI): m/z 977.3 [M+H]⁺

### Step 2: N-(6-(1,1-dioxido-4-oxo-1,2,5-thiadiazolidin-2-yl)-5-fluoro-7-hydroxynaphthalen-2-yl)-2-((2S)-4-(1-(2,6-dioxopiperidin-3-yl)-3-methyl-2-oxo-2,3-dihydro-1H-benzo[d]imidazol-5-yl)-2-methylpiperazin-1-yl)acetamide (Degrader 90)

To a solution of N-[7-benzyloxy-5-fluoro-6-(1,1,4-trioxo-1,2,5-thiadiazolidin-2-yl)-2-naphthyl]-2-[(2S)-4-[1-(2,6-dibenzyloxy-3-pyridyl)-3-methyl-2-oxo-benzimidazol-5-yl]-2-methyl-piperazin-1-yl]acetamide (3, 80 mg, 81.88 μmol) in DMF (4 mL) was added Pd/C (80 mg, 10% purity) and Pd(OH)₂ (80 mg, 10% purity) under N₂ atmosphere. The suspension was degassed and purged with H₂ 3 times. The mixture was stirred under H₂ (15 Psi) at 25° C. for 16 h. The reaction mixture was filtered and concentrated under vacuum. The residue was purified by prep-HPLC (flow: 25 mL/min; gradient: from 8-38% MeCN in water (0.1% TFA) over 12 min; column: Phenomenex Synergi C18 150×25 mm×10 um) to afford N-(6-(1,1-dioxido-4-oxo-1,2,5-thiadiazolidin-2-yl)-5-fluoro-7-hydroxynaphthalen-2-yl)-2-((2S)-4-(1-(2,6-dioxopiperidin-3-yl)-3-methyl-2-oxo-2,3-dihydro-1H-benzo[d]imidazol-5-yl)-2-methylpiperazin-1-yl)acetamide (Degrader 90, 36.04 mg, 43.80 μmol, 54% yield, TFA salt) as a whit solid.

LCMS (ESI): m/z 709.1 [M+H]⁺

¹H NMR (400 MHz, DMSO-d₆) δ11.08 (s, 1H), 10.93-10.72 (m, 1H), 9.91 (br s, 2H), 8.12 (s, 1H), 7.91 (d, J=9.2 Hz, 1H), 7.49 (br d, J=9.2 Hz, 1H), 7.01 (m, 2H), 6.93 (d, J=1.6 Hz, 1H), 6.71 (br d, J=8.0 Hz, 1H), 5.32 (m, 1H), 4.65-4.45 (m, 1H), 4.13 (s, 2H), 3.84-3.77 (m, 1H), 3.63-3.51 (m, 3H), 3.48-3.40 (m, 2H), 3.33 (s, 3H), 3.20-2.94 (m, 2H), 2.91-2.84 (m, 1H), 2.71-2.61 (m, 2H), 2.05-1.94 (m, 1H), 1.48-1.32 (m, 3H).

### N-(6-(1,1-dioxido-4-oxo-1,2,5-thiadiazolidin-2-yl)-5-fluoro-7-hydroxynaphthalen-2-yl)-2-((2R)-4-(1-(2,6-dioxopiperidin-3-yl)-3-methyl-2-oxo-2,3-dihydro-1H-benzo[d]imidazol-5-yl)-2-methylpiperazin-1-yl)acetamide (Degrader 91)

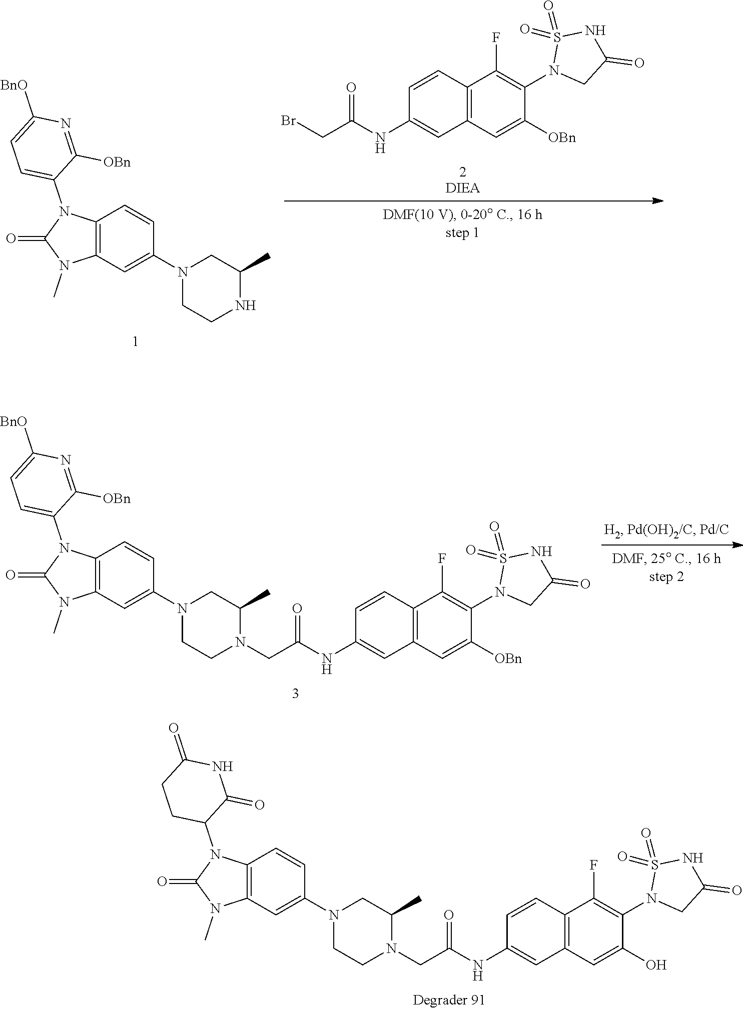 [SMILES: C[C@@H]1CN(c2ccc3c(c2)n(C)c(=O)n3-c2ccc(OCc3ccccc3)nc2OCc2ccccc2)CCN1 ; O=C(CBr)Nc1ccc2c(F)c(N3CC(=O)NS3(=O)=O)c(OCc3ccccc3)cc2c1 ; C[C@@H]1CN(c2ccc3c(c2)n(C)c(=O)n3-c2ccc(OCc3ccccc3)nc2OCc2ccccc2)CCN1CC(=O)Nc1ccc2c(F)c(N3CC(=O)NS3(=O)=O)c(OCc3ccccc3)cc2c1 ; C[C@@H]1CN(c2ccc3c(c2)n(C)c(=O)n3C2CCC(=O)NC2=O)CCN1CC(=O)Nc1ccc2c(F)c(N3CC(=O)NS3(=O)=O)c(O)cc2c1]

### Step 1: (R)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-2-methylpiperazin-1-yl)acetamide (3)

To a mixture of 1-(2,6-dibenzyloxy-3-pyridyl)-3-methyl-5-[(3R)-3-methylpiperazin-1-yl]benzimidazol-2-one (1, 160 mg, 298.71 μmol) and N-(7-(benzyloxy)-6-(1,1-dioxido-4-oxo-1,2,5-thiadiazolidin-2-yl)-5-fluoronaphthalen-2-yl)-2-bromoacetamide (2, 172 mg, 329.29 μmol) in DMF (2 mL) was added DIPEA (193.03 mg, 1.49 mmol, 260.15 uL). The mixture was stirred at 15° C. for 16 h. The reaction mixture was filtered and purified by reversed phase HPLC (NH₃·H₂O) and then lyophilisation to give the (R)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-2-methylpiperazin-1-yl)acetamide (3, 100 mg, 98.25 μmol, 33% yield) as off-white solid LCMS (ESI): m/z 977.3 [M+H]⁺

### Step 2: N-(6-(1,1-dioxido-4-oxo-1,2,5-thiadiazolidin-2-yl)-5-fluoro-7-hydroxynaphthalen-2-yl)-2-((2R)-4-(1-(2,6-dioxopiperidin-3-yl)-3-methyl-2-oxo-2,3-dihydro-1H-benzo[d]imidazol-5-yl)-2-methylpiperazin-1-yl)acetamide (Degrader 91)

To a solution of N-[7-benzyloxy-5-fluoro-6-(1,1,4-trioxo-1,2,5-thiadiazolidin-2-yl)-2-naphthyl]-2-[(2R)-4-[1-(2,6-dibenzyloxy-3-pyridyl)-3-methyl-2-oxo-benzimidazol-5-yl]-2-methyl-piperazin-1-yl]acetamide (3, 80 mg, 81.88 μmol) in DMF (4 mL) was added Pd/C (80 mg, 19.94 μmol, 10% purity) and Pd(OH)₂ (80 mg, 19.94 μmol, 10% purity) under N₂ atmosphere. The suspension was degassed and purged with H₂ 3 times. The mixture was stirred under H₂ (15 Psi) at 25° C. for 16 h. The reaction mixture was filtered and concentrated under vacuum. The residue was purified by prep-HPLC (flow: 25 mL/min; gradient: from 8-38% MeCN in water (0.1% TFA) over 12 min; column: Phenomenex Synergi C18 150×25 mm×10 um) to afford N-(6-(1,1-dioxido-4-oxo-1,2,5-thiadiazolidin-2-yl)-5-fluoro-7-hydroxynaphthalen-2-yl)-2-((2R)-4-(1-(2,6-dioxopiperidin-3-yl)-3-methyl-2-oxo-2,3-dihydro-1H-benzo[d]imidazol-5-yl)-2-methylpiperazin-1-yl)acetamide (Degrader 91, 32.8 mg, 39.87 μmol, 49% yield, TFA salt) as a white solid.

LCMS (ESI): m/z 709.1 [M+H]⁺

¹H NMR (400 MHz, DMSO-d₆) δ 11.08 (s, 1H), 10.93-10.72 (m, 1H), 9.91 (br s, 2H), 8.12 (s, 1H), 7.91 (d, J=9.2 Hz, 1H), 7.49 (br d, J=9.2 Hz, 1H), 7.01 (m, 2H), 6.93 (d, J=1.6 Hz, 1H), 6.71 (br d, J=8.0 Hz, 1H), 5.32 (m, 1H), 4.65-4.45 (m, 1H), 4.13 (s, 2H), 3.84-3.77 (m, 1H), 3.63-3.51 (m, 3H), 3.48-3.40 (m, 2H), 3.33 (s, 3H), 3.20-2.94 (m, 2H), 2.91-2.84 (m, 1H), 2.71-2.61 (m, 2H), 2.05-1.94 (m, 1H), 1.48-1.32 (m, 3H).

### N-(6-(1,1-dioxido-4-oxo-1,2,5-thiadiazolidin-2-yl)-5-fluoro-7-hydroxynaphthalen-2-yl)-2-(2-(1-(2,6-dioxopiperidin-3-yl)-3-methyl-2-oxo-2,3-dihydro-1H-benzo[d]imidazol-5-yl)-2,7-diazaspiro[3.5]nonan-7-yl)acetamide (Degrader 92)

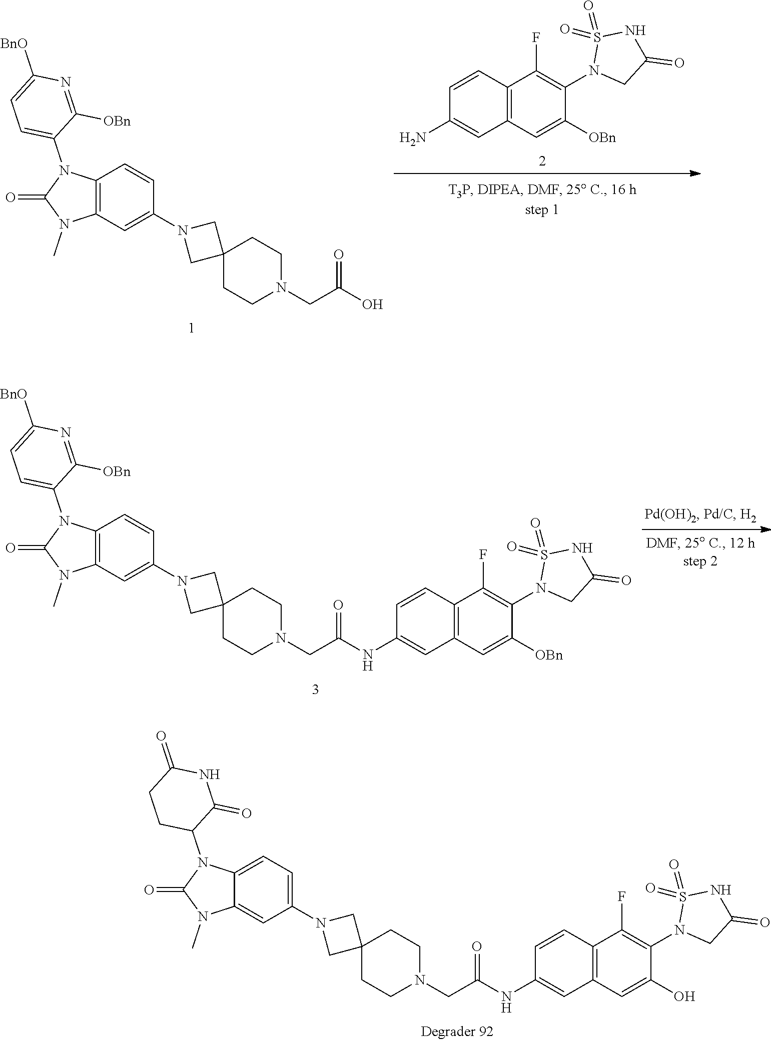 [SMILES: Cn1c(=O)n(-c2ccc(OCc3ccccc3)nc2OCc2ccccc2)c2ccc(N3CC4(CCN(CC(=O)O)CC4)C3)cc21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(N3CC4(CCN(CC(=O)Nc5ccc6c(F)c(N7CC(=O)NS7(=O)=O)c(OCc7ccccc7)cc6c5)CC4)C3)cc21 ; Cn1c(=O)n(C2CCC(=O)NC2=O)c2ccc(N3CC4(CCN(CC(=O)Nc5ccc6c(F)c(N7CC(=O)NS7(=O)=O)c(O)cc6c5)CC4)C3)cc21]

### Step 1: N-(7-(benzyloxy)-6-(1,1-dioxido-4-oxo-1,2,5-thiadiazolidin-2-yl)-5-fluoronaphthalen-2-yl)-2-(2-(1-(2,6-bis(benzyloxy)pyridin-3-yl)-3-methyl-2-oxo-2,3-dihydro-1H-benzo[d]imidazol-5-yl)-2,7-diazaspiro[3.5]nonan-7-yl)acetamide (3)

To a solution of 2-[2-[1-(2,6-dibenzyloxy-3-pyridyl)-3-methyl-2-oxo-benzimidazol-5-yl]-2,7-diazaspiro[3.5]nonan-7-yl]acetic acid (1, 150 mg, 242.05 μmol) and 5-(6-amino-3-(benzyloxy)-1-fluoronaphthalen-2-yl)-1,2,5-thiadiazolidin-3-one 1,1-dioxide (2, 165.00 mg, 320.12 μmol, TFA salt) in DMF (1.5 mL) were added DIPEA (156.42 mg, 1.21 mmol, 210.80 μL) and T₃P (462.09 mg, 726.15 μmol, 50% purity). The mixture was stirred at 20° C. for 16 h. The reaction mixture was filtered. The mixture was purified by prep-HPLC (flow: 25 mL/min; gradient: from water (0.05% ammonia hydroxide v/v)-CAN over 9 min; column: Waters Xbridge 150*25 mm*5 um) to afford N-(7-(benzyloxy)-6-(1,1-dioxido-4-oxo-1,2,5-thiadiazolidin-2-yl)-5-fluoronaphthalen-2-yl)-2-(2-(1-(2,6-bis(benzyloxy)pyridin-3-yl)-3-methyl-2-oxo-2,3-dihydro-1H-benzo[d]imidazol-5-yl)-2,7-diazaspiro[3.5]nonan-7-yl)acetamide (3, 110 mg, 109.66 μmol, 45% yield) as white solid.

LCMS (ESI): m/z 1003.0 [M+H]⁺

### Step 2: N-(6-(1,1-dioxido-4-oxo-1,2,5-thiadiazolidin-2-yl)-5-fluoro-7-hydroxynaphthalen-2-yl)-2-(2-(1-(2,6-dioxopiperidin-3-yl)-3-methyl-2-oxo-2,3-dihydro-1H-benzo[d]imidazol-5-yl)-2,7-diazaspiro[3.5]nonan-7-yl)acetamide (Degrader 92)

To a solution of N-[7-benzyloxy-5-fluoro-6-(1,1,4-trioxo-1,2,5-thiadiazolidin-2-yl)-2-naphthyl]-2-[2-[1-(2,6-dibenzyloxy-3-pyridyl)-3-methyl-2-oxo-benzimidazol-5-yl]-2,7-diazaspiro[3.5]nonan-7-yl]acetamide (3, 110 mg, 109.66 μmol) in DMF (4 mL) was added Pd/C (110 mg, 10% purity) and Pd(OH)₂ (110 mg, 109.66 μmol, 10% purity) under N₂ atmosphere. The suspension was degassed and purged with H₂ 3 times. The mixture was stirred under H₂ (15 Psi) at 25° C. for 12 h. The reaction mixture was filtered and concentrated under vacuum. The residue was purified by prep-HPLC (flow: 25 mL/min; gradient: from 8-38% MeCN in water (0.1% TFA) over 12 min; column: Phenomenex Synergi C18 150×25 mm×10 um) to afford N-(6-(1,1-dioxido-4-oxo-1,2,5-thiadiazolidin-2-yl)-5-fluoro-7-hydroxynaphthalen-2-yl)-2-(2-(1-(2,6-dioxopiperidin-3-yl)-3-methyl-2-oxo-2,3-dihydro-1H-benzo[d]imidazol-5-yl)-2,7-diazaspiro[3.5]nonan-7-yl)acetamide (Degrader 92, 67.2 mg, 79.17 μmol, 72% yield, TFA salt) as a white solid.

LCMS (ESI): m/z 735.1 [M+H]⁺

¹H NMR (400 MHz, DMSO-d₆) δ 11.06 (s, 1H), 10.81 (br d, J=6.0 Hz, 1H), 10.38-10.16 (m, 1H), 9.93-9.75 (m, 1H), 8.12 (s, 1H), 7.92 (d, J=8.8 Hz, 1H), 7.47 (br d, J=9.2 Hz, 1H), 7.02 (s, 1H), 6.93 (d, J=8.4 Hz, 1H), 6.30 (s, 1H), 6.12 (br d, J=8.8 Hz, 1H), 5.28 (m, 1H), 4.34-4.24 (m, 2H), 4.24-4.16 (m, 2H), 3.72-3.64 (m, 2H), 3.62-3.49 (m, 4H), 3.29 (s, 3H), 3.20 (m, 2H), 2.97-2.80 (m, 1H), 2.75-2.60 (m, 2H), 2.10-1.96 (m, 4H).

### N-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)piperazin-1-yl)propanamide (Degrader 93)

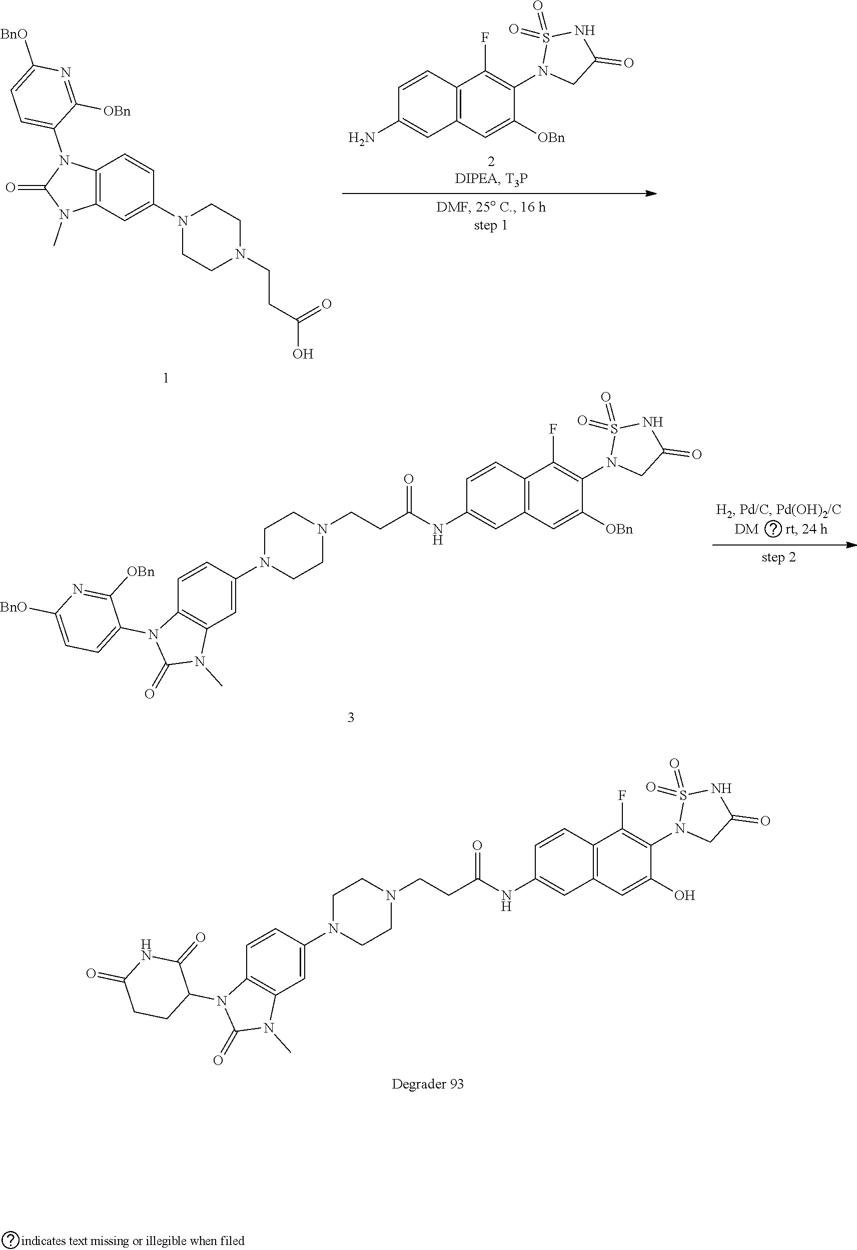 [SMILES: Cn1c(=O)n(-c2ccc(OCc3ccccc3)nc2OCc2ccccc2)c2ccc(N3CCN(CCC(=O)O)CC3)cc21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(N3CCN(CCC(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(N3CCN(CCC(=O)Nc4ccc5c(F)c(N6CC(=O)NS6(=O)=O)c(O)cc5c4)CC3)cc21]

### Step 1: N-(7-(benzyloxy)-6-(1,1-dioxido-4-oxo-1,2,5-thiadiazolidin-2-yl)-5-fluoronaphthalen-2-yl)-3-(4-(1-(2,6-bis(benzyloxy)pyridin-3-yl)-3-methyl-2-oxo-2,3-dihydro-1H-benzo[d]imidazol-5-yl)piperazin-1-yl)propanamide (3)

To a solution of 3-(4-(1-(2,6-bis(benzyloxy)pyridin-3-yl)-3-methyl-2-oxo-2,3-dihydro-1H-benzo[d]imidazol-5-yl)piperazin-1-yl)propanoic acid (1, 150 mg, 252.67 μmol) and 5-(6-amino-3-(benzyloxy)-1-fluoronaphthalen-2-yl)-1,2,5-thiadiazolidin-3-one 1,1-dioxide (2, 169.30 mg, 328.46 μmol, 061) in DMF (1.5 mL) were added DIPEA (163.28 mg, 1.26 mmol, 220.05 uL) and T₃P (482.36 mg, 758.00 μmol, 50% purity). The mixture was stirred at 20° C. for 16 h. The mixture was purified by Prep-HPLC (NH₃·H₂O) and then lyophilization. The N-(7-(benzyloxy)-6-(1,1-dioxido-4-oxo-1,2,5-thiadiazolidin-2-yl)-5-fluoronaphthalen-2-yl)-3-(4-(1-(2,6-bis(benzyloxy)pyridin-3-yl)-3-methyl-2-oxo-2,3-dihydro-1H-benzo[d]imidazol-5-yl)piperazin-1-yl)propanamide (3, 110 mg, 112.58 μmol, 45% yield) as white solid.

LCMS (ESI): m/z 977.1 [M+H]⁺

### Step 2: N-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)piperazin-1-yl)propanamide (Degrader 93)

To a solution of N-[7-benzyloxy-5-fluoro-6-(1,1,4-trioxo-1,2,5-thiadiazolidin-2-yl)-2-naphthyl]-3-[4-[1-(2,6-dibenzyloxy-3-pyridyl)-3-methyl-2-oxo-benzimidazol-5-yl]piperazin-1-yl]propanamide (3, 110 mg, 112.58 μmol) in DMF (4 mL) was added Pd/C (110 mg, 10% purity) and Pd(OH)₂/C (110 mg, 112.58 μmol, 10% purity) under N₂ atmosphere. The suspension was degassed and purged with H₂ 3 times. The mixture was stirred under H₂ (15 Psi) at 25° C. for 12 h. The reaction mixture was filtered and concentrated under vacuum. The residue was purified by prep-HPLC (flow: 25 mL/min; gradient: from 8-38% MeCN in water (0.1% TFA) over 12 min; column: Phenomenex Synergi C18 150×25 mm×10 um) to afford N-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)piperazin-1-yl)propanamide (Degrader 93, 27.21 mg, 33.07 μmol, 29% yield, TFA salt) as a white solid.

LCMS (ESI): m/z 709.1 [M+H]⁺

¹H NMR (400 MHz, DMSO-d₆) δ 11.08 (s, 1H), 10.43 (s, 1H), 9.82 (br s, 1H), 9.48 (br d, J=1.2 Hz, 1H), 8.15 (s, 1H), 7.86 (d, J=8.8 Hz, 1H), 7.50-7.41 (m, 1H), 7.00 (d, J=8.4 Hz, 1H), 6.97-6.92 (m, 2H), 6.71 (m, 1H), 5.31 (m, 1H), 4.11 (s, 2H), 3.79 (br d, J=12.0 Hz, 2H), 3.64 (br d, J=9.6 Hz, 2H), 3.54 (br s, 3H), 3.48-3.37 (m, 3H), 3.27 (m, 2H), 3.05-2.97 (m, 2H), 2.96-2.85 (m, 2H), 2.76-2.62 (m, 2H), 2.03-1.93 (m, 1H).

### N-(6-(1,1-dioxido-4-oxo-1,2,5-thiadiazolidin-2-yl)-5-fluoro-7-hydroxynaphthalen-2-yl)-2-(4-((1-(2,6-dioxopiperidin-3-yl)-3-methyl-2-oxo-2,3-dihydro-1H-benzo[d]imidazol-5-yl)amino)-3-methylphenyl)propanamide (Degrader 94)

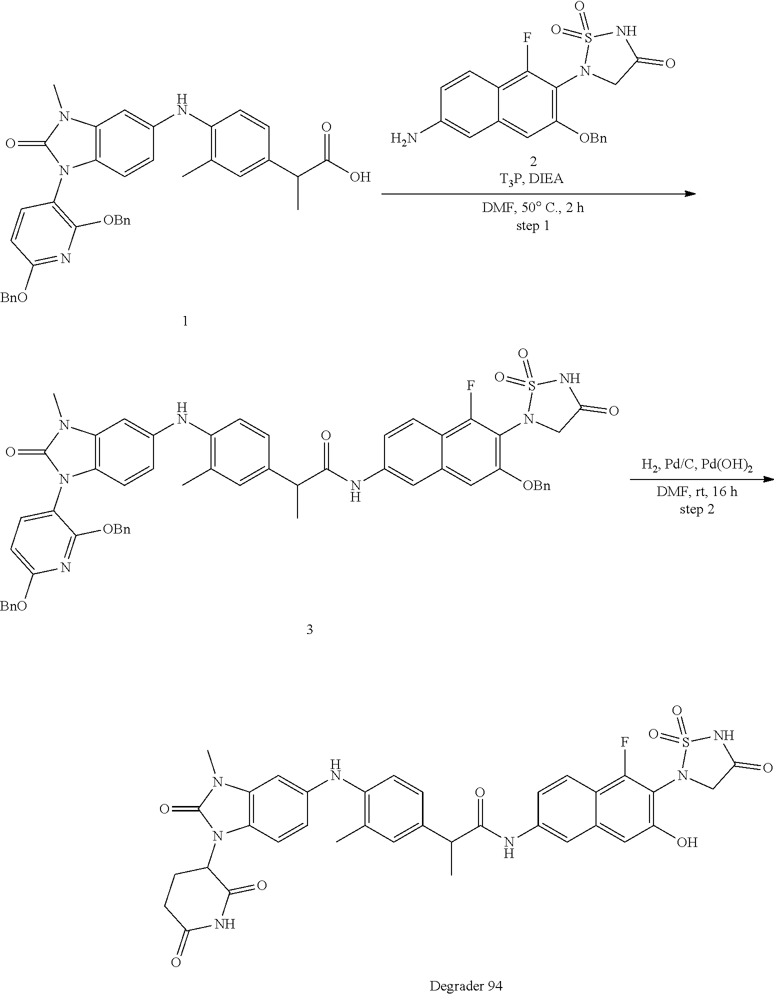 [SMILES: Cc1cc(C(C)C(=O)O)ccc1Nc1ccc2c(c1)n(C)c(=O)n2-c1ccc(OCc2ccccc2)nc1OCc1ccccc1 ; Nc1ccc2c(F)c(N3CC(=O)NS3(=O)=O)c(OCc3ccccc3)cc2c1 ; Cc1cc(C(C)C(=O)Nc2ccc3c(F)c(N4CC(=O)NS4(=O)=O)c(OCc4ccccc4)cc3c2)ccc1Nc1ccc2c(c1)n(C)c(=O)n2-c1ccc(OCc2ccccc2)nc1OCc1ccccc1 ; Cc1cc(C(C)C(=O)Nc2ccc3c(F)c(N4CC(=O)NS4(=O)=O)c(O)cc3c2)ccc1Nc1ccc2c(c1)n(C)c(=O)n2C1CCC(=O)NC1=O]

### Step 1: N-(7-(benzyloxy)-6-(1,1-dioxido-4-oxo-1,2,5-thiadiazolidin-2-yl)-5-fluoronaphthalen-2-yl)-2-(4-((1-(2,6-bis(benzyloxy)pyridin-3-yl)-3-methyl-2-oxo-2,3-dihydro-1H-benzo[d]imidazol-5-yl)amino)-3-methylphenyl)propanamide (3)

To a solution of 2-[4-[[1-(2,6-dibenzyloxy-3-pyridyl)-3-methyl-2-oxo-benzimidazol-5-yl]amino]-3-methyl-phenyl]propanoic acid (1, 150 mg, 244.03 μmol) and 5-(6-amino-3-(benzyloxy)-1-fluoronaphthalen-2-yl)-1,2,5-thiadiazolidin-3-one 1,1-dioxide (2, 164 mg, 318.18 μmol, TFA salt) in DMF (1.5 mL) were added DIPEA (158 mg, 1.22 mmol, 212.94 μL) and T₃P (466 mg, 732.29 μmol, 50% purity). The mixture was stirred at 50° C. for 2 h. The reaction was quenched by water (1 mL) to get a mixture. The mixture was purified by reversed phase HPLC (NH₃·H₂O) and concentrated to get N-(7-(benzyloxy)-6-(1,1-dioxido-4-oxo-1,2,5-thiadiazolidin-2-yl)-5-fluoronaphthalen-2-yl)-2-(4-((1-(2,6-bis(benzyloxy)pyridin-3-yl)-3-methyl-2-oxo-2,3-dihydro-1H-benzo[d]imidazol-5-yl)amino)-3-methylphenyl)propanamide (3, 50 mg, 48.09 μmol, 20% yield) as yellow solid.

LCMS (ESI): m/z 998.1 [M+H]⁺

### Step 2: N-(6-(1,1-dioxido-4-oxo-1,2,5-thiadiazolidin-2-yl)-5-fluoro-7-hydroxynaphthalen-2-yl)-2-(4-((1-(2,6-dioxopiperidin-3-yl)-3-methyl-2-oxo-2,3-dihydro-1H-benzo[d]imidazol-5-yl)amino)-3-methylphenyl)propanamide (Degrader 94)

To a solution of N-[7-benzyloxy-5-fluoro-6-(1,1,4-trioxo-1,2,5-thiadiazolidin-2-yl)-2-naphthyl]-2-[4-[[1-(2,6-dibenzyloxy-3-pyridyl)-3-methyl-2-oxo-benzimidazol-5-yl]amino]-3-methyl-phenyl]propanamide (3, 50 mg, 50.10 μmol) in DMF (2 mL) was added Pd/C (50 mg, 10% purity) and Pd(OH)₂ (50 mg, 50.10 μmol, 10% purity) under N₂ atmosphere. The suspension was degassed and purged with H₂ 3 times. The mixture was stirred under H₂ (15 Psi) at 25° C. for 12 h.

The reaction mixture was filtered and concentrated under vacuum. The residue was purified by prep-HPLC (flow: 25 mL/min; gradient: from 8-38% MeCN in water (0.1% TFA) over 12 min; column: Phenomenex SynergiC18 150×25 mm×10 um) to afford N-(6-(1,1-dioxido-4-oxo-1,2,5-thiadiazolidin-2-yl)-5-fluoro-7-hydroxynaphthalen-2-yl)-2-(4-((1-(2,6-dioxopiperidin-3-yl)-3-methyl-2-oxo-2,3-dihydro-1H-benzo[d]imidazol-5-yl)amino)-3-methylphenyl)propanamide (Degrader 94, 26.19 mg, 29.80 μmol, 59% yield, TFA salt) as a blue solid.

LCMS (ESI): m/z 730.1 [M+H]⁺

¹H NMR (400 MHz, DMSO-d₆) δ 11.07 (s, 1H), 10.53-10.31 (m, 1H), 10.23 (s, 1H), 8.20 (s, 1H), 7.84 (d, J=8.8 Hz, 1H), 7.52-7.40 (m, 1H), 7.19 (d, J=1.6 Hz, 1H), 7.12-7.07 (m, 1H), 7.06-7.02 (m, 1H), 6.97-6.93 (m, 2H), 6.79 (d, J=2.0 Hz, 1H), 6.66 (m, 1H), 5.30 (m, 1H), 4.44-4.36 (m, 2H), 3.76-3.73 (m, 1H), 3.25 (s, 3H), 2.95-2.84 (m, 1H), 2.74-2.63 (m, 2H), 2.21 (s, 3H), 2.04-1.96 (m, 1H), 1.41 (d, J=6.8 Hz, 3H).

### N-(6-(1,1-dioxido-4-oxo-1,2,5-thiadiazolidin-2-yl)-5-fluoro-7-hydroxynaphthalen-2-yl)-2-(5-(1-(2,6-dioxopiperidin-3-yl)-3-methyl-2-oxo-2,3-dihydro-1H-benzo[d]imidazol-5-yl)-3-methyl-1H-indazol-1-yl)acetamide (Degrader 95)

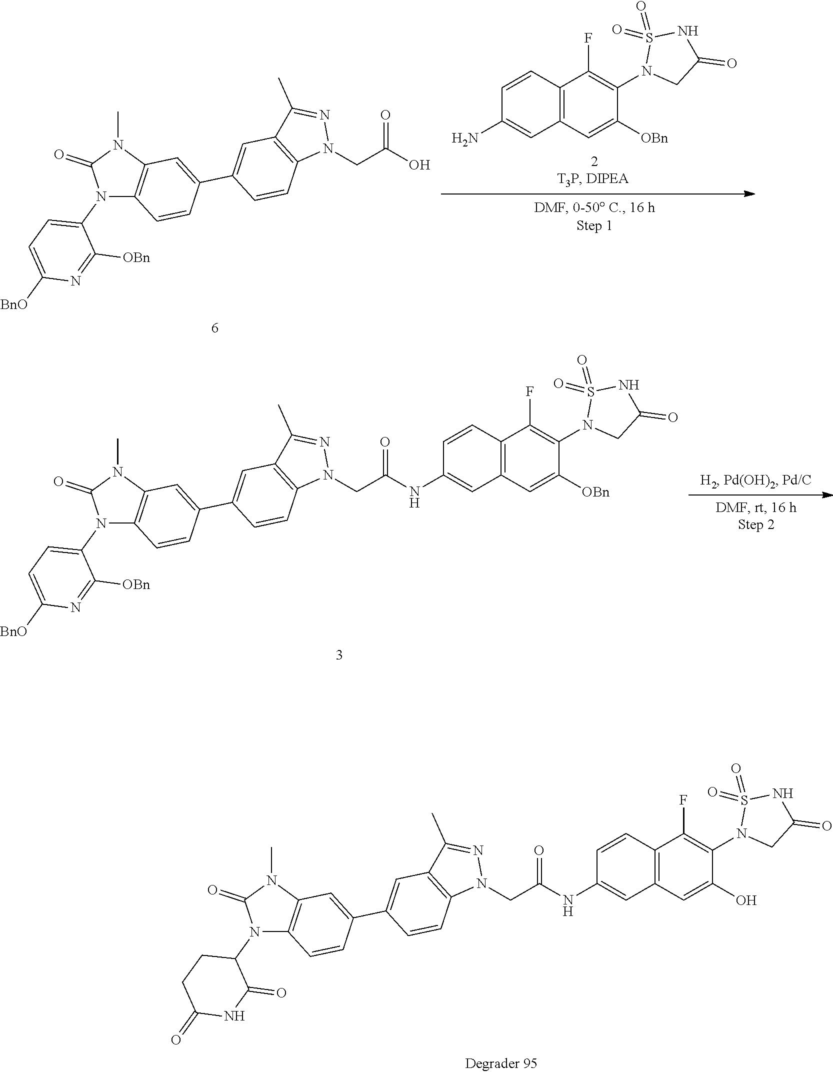 [SMILES: Cc1nn(CC(=O)O)c2ccc(-c3ccc4c(c3)n(C)c(=O)n4-c3ccc(OCc4ccccc4)nc3OCc3ccccc3)cc12 ; Nc1ccc2c(F)c(N3CC(=O)NS3(=O)=O)c(OCc3ccccc3)cc2c1 ; Cc1nn(CC(=O)Nc2ccc3c(F)c(N4CC(=O)NS4(=O)=O)c(OCc4ccccc4)cc3c2)c2ccc(-c3ccc4c(c3)n(C)c(=O)n4-c3ccc(OCc4ccccc4)nc3OCc3ccccc3)cc12 ; Cc1nn(CC(=O)Nc2ccc3c(F)c(N4CC(=O)NS4(=O)=O)c(O)cc3c2)c2ccc(-c3ccc4c(c3)n(C)c(=O)n4C3CCC(=O)NC3=O)cc12]

### Step 1: N-(7-(benzyloxy)-6-(1,1-dioxido-4-oxo-1,2,5-thiadiazolidin-2-yl)-5-fluoronaphthalen-2-yl)-2-(5-(1-(2,6-bis(benzyloxy)pyridin-3-yl)-3-methyl-2-oxo-2,3-dihydro-1H-benzo[d]imidazol-5-yl)-3-methyl-1H-indazol-1-yl)acetamide (3)

To a solution of 2-(5-(1-(2,6-bis(benzyloxy)pyridin-3-yl)-3-methyl-2-oxo-2,3-dihydro-1H-benzo[d]imidazol-5-yl)-3-methyl-1H-indazol-1-yl)acetic acid (1, 150 mg, 239.74 μmol, 1 eq) and 5-(6-amino-3-(benzyloxy)-1-fluoronaphthalen-2-yl)-1,2,5-thiadiazolidin-3-one 1,1-dioxide (2, 148.00 mg, 287.69 μmol, 1.2 eq, TFA salt) in DMF (1.5 mL mL) were added DIPEA (464.76 mg, 3.60 mmol, 626.37 μL, 15 eq) and T₃P (1.07 g, 1.68 mmol, 50% purity, 7 eq) at 0° C. The mixture was stirred at 50° C. for 16 h. The reaction mixture was washed with water (3 mL) and extracted with EA (3 mL*3) and concentrated under reduced pressure to give a residue. The residue was purified by reversed-phase (0.1% of NH₃·H₂O) and the eluent was lyophilized to give N-(7-(benzyloxy)-6-(1,1-dioxido-4-oxo-1,2,5-thiadiazolidin-2-yl)-5-fluoronaphthalen-2-yl)-2-(5-(1-(2,6-bis(benzyloxy)pyridin-3-yl)-3-methyl-2-oxo-2,3-dihydro-1H-benzo[d]imidazol-5-yl)-3-methyl-1H-indazol-1-yl)acetamide (3, 80 mg, 73.73 μmol, 31% yield) as yellow solid.

LCMS (ESI): m/z 1009.2 [M+H]⁺

### Step 2: N-(6-(1,1-dioxido-4-oxo-1,2,5-thiadiazolidin-2-yl)-5-fluoro-7-hydroxynaphthalen-2-yl)-2-(5-(1-(2,6-dioxopiperidin-3-yl)-3-methyl-2-oxo-2,3-dihydro-1H-benzo[d]imidazol-5-yl)-3-methyl-1H-indazol-1-yl)acetamide (Degrader 95)

To a solution of N-(7-(benzyloxy)-6-(1,1-dioxido-4-oxo-1,2,5-thiadiazolidin-2-yl)-5-fluoronaphthalen-2-yl)-2-(5-(1-(2,6-bis(benzyloxy)pyridin-3-yl)-3-methyl-2-oxo-2,3-dihydro-1H-benzo[d]imidazol-5-yl)-3-methyl-1H-indazol-1-yl)acetamide (3, 80 mg, 79.28 μmol, 1 eq) in DMF (2 mL) were added Pd/C (80 mg, 10% purity) and Pd(OH)₂/C (80 mg, 10% purity) under N₂ atmosphere. The suspension was degassed and purged with H₂ 3 times. The mixture was stirred under H₂ (15 psi) at 20° C. for 16 h. The mixture was filtered and concentrated in vacuum. The residue was purified by prep-HPLC (flow: 25 mL/min; gradient: from 30-60% MeCN in water (0.1% TFA) over 9 min; column: 3_Phenomenex Luna C18 75*30 mm*3 um) to afford N-(6-(1,1-dioxido-4-oxo-1,2,5-thiadiazolidin-2-yl)-5-fluoro-7-hydroxynaphthalen-2-yl)-2-(5-(1-(2,6-dioxopiperidin-3-yl)-3-methyl-2-oxo-2,3-dihydro-1H-benzo[d]imidazol-5-yl)-3-methyl-1H-indazol-1-yl)acetamide (Degrader 95, 20.18 mg, 26.97 μmol, 34% yield) as a white solid.

LCMS (ESI): m/z 741.5 [M+H]⁺

¹H NMR (400 MHz, DMSO-d₆) δ=11.13 (s, 1H), 10.65 (s, 1H), 10.43-10.23 (m, 1H), 8.16 (s, 1H), 8.02 (s, 1H), 7.89 (d, J=8.8 Hz, 1H), 7.78-7.73 (m, 1H), 7.72-7.66 (m, 1H), 7.58 (s, 1H), 7.49-7.38 (m, 2H), 7.21 (d, J=8.0 Hz, 1H), 6.94 (s, 1H), 5.48-5.36 (m, 1H), 5.32 (s, 2H), 4.34 (s, 2H), 3.44 (s, 3H), 2.99-2.88 (m, 2H), 2.81-2.76 (m, 1H), 2.56 (s, 3H), 2.11-2.04 (m, 1H)

### N-(6-(1,1-dioxido-4-oxo-1,2,5-thiadiazolidin-2-yl)-5-fluoro-7-hydroxynaphthalen-2-yl)-2-((2-(1-(2,6-dioxopiperidin-3-yl)-3-methyl-2-oxo-2,3-dihydro-1H-benzo[d]imidazol-5-yl)-2-azaspiro[3.3]heptan-6-yl)oxy)acetamide (Degrader 96)

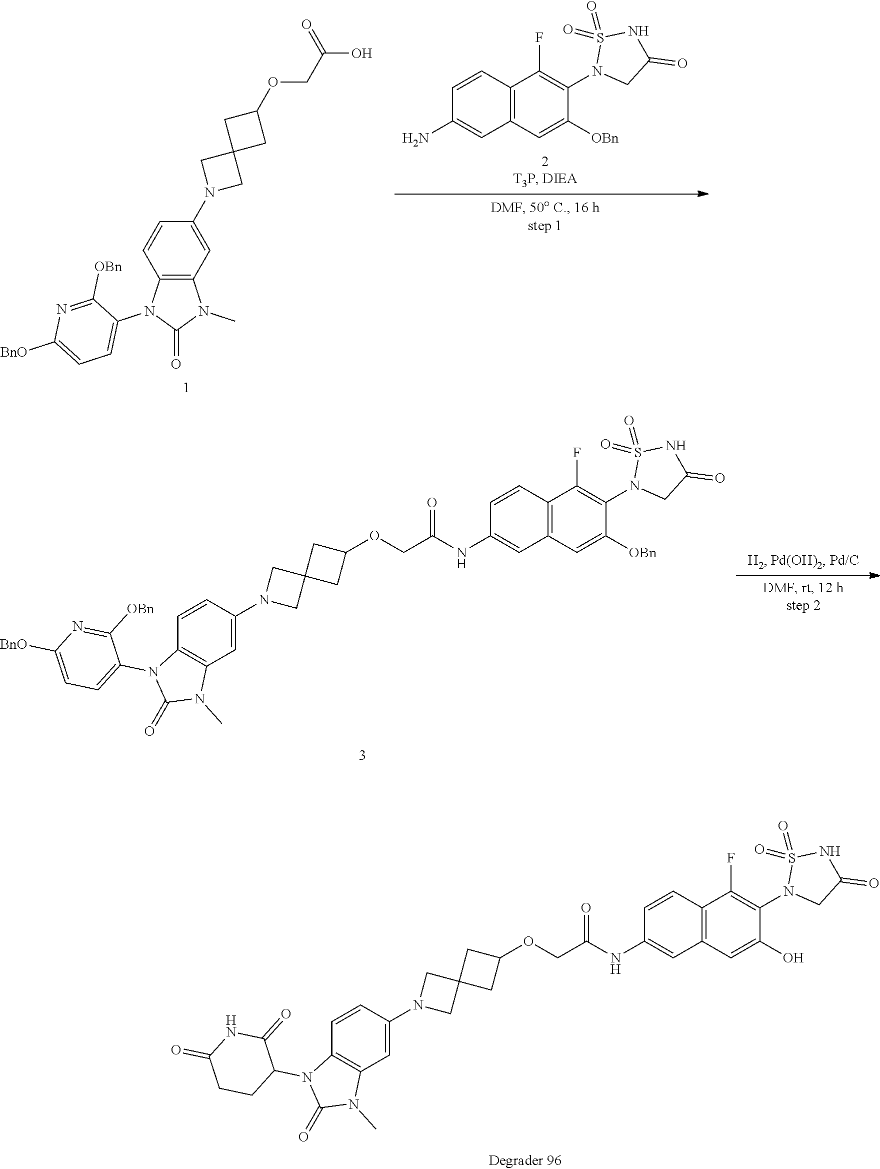 [SMILES: Cn1c(=O)n(-c2ccc(OCc3ccccc3)nc2OCc2ccccc2)c2ccc(N3CC4(CC(OCC(=O)O)C4)C3)cc21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(N3CC4(CC(OCC(=O)Nc5ccc6c(F)c(N7CC(=O)NS7(=O)=O)c(OCc7ccccc7)cc6c5)C4)C3)cc21 ; Cn1c(=O)n(C2CCC(=O)NC2=O)c2ccc(N3CC4(CC(OCC(=O)Nc5ccc6c(F)c(N7CC(=O)NS7(=O)=O)c(O)cc6c5)C4)C3)cc21]

### Step 1: N-(7-(benzyloxy)-6-(1,1-dioxido-4-oxo-1,2,5-thiadiazolidin-2-yl)-5-fluoronaphthalen-2-yl)-2-((2-(1-(2,6-bis(benzyloxy)pyridin-3-yl)-3-methyl-2-oxo-2,3-dihydro-1H-benzo[d]imidazol-5-yl)-2-azaspiro[3.3]heptan-6-yl)oxy)acetamide (3)

DIPEA (237.62 mg, 1.84 mmol, 320.24 μL, 15 eq) in DMF (1 mL) was added 2-((2-(1-(2,6-bis(benzyloxy)pyridin-3-yl)-3-methyl-2-oxo-2,3-dihydro-1H-benzo[d]imidazol-5-yl)-2-azaspiro[3.3]heptan-6-yl)oxy)acetic acid (1, 80 mg, 122.57 μmol, formic acid salt) and 5-(6-amino-3-(benzyloxy)-1-fluoronaphthalen-2-yl)-1,2,5-thiadiazolidin-3-one 1,1-dioxide (2, 69.49 mg, 134.83 μmol) at 0° C. Then T₃P (545.99 mg, 857.99 μmol, 50% purity) was added at 0° C. The reaction mixture was stirred at 50° C. for 12 hrs. The mixture was concentrated under vacuum. The crude product was purified by reversed-phase HPLC (0.1% NH₃H₂O) and concentrated under vacuum to remove MeCN. The liquid was under lyophilization to afford N-(7-(benzyloxy)-6-(1,1-dioxido-4-oxo-1,2,5-thiadiazolidin-2-yl)-5-fluoronaphthalen-2-yl)-2-((2-(1-(2,6-bis(benzyloxy)pyridin-3-yl)-3-methyl-2-oxo-2,3-dihydro-1H-benzo[d]imidazol-5-yl)-2-azaspiro[3.3]heptan-6-yl)oxy)acetamide (3, 0.1 g, 101.00 μmol, 82% yield) as a white solid.

LCMS (ESI): m/z 990.7 [M+H]+

¹H NMR (400 MHZ, DMSO-d6) δ=9.99 (s, 1H), 8.30 (s, 1H), 8.23-8.10 (m, 1H), 7.89 (d, J=8.9 Hz, 1H), 7.75 (d, J=8.3 Hz, 1H), 7.63-7.54 (m, 3H), 7.46-7.18 (m, 15H), 6.58 (d, J=8.3 Hz, 1H), 6.49 (d, J=8.3 Hz, 1H), 6.30 (d, J=1.9 Hz, 1H), 6.04 (dd, J=2.0, 8.4 Hz, 1H), 5.42-5.30 (m, 4H), 5.27 (s, 2H), 4.12-3.98 (m, 5H), 3.76 (d, J=8.9 Hz, 4H), 3.67-3.54 (m, 2H), 3.14 (dq, J=4.1, 7.3 Hz, 2H), 2.27-2.16 (m, 2H).

### Step 2: N-(6-(1,1-dioxido-4-oxo-1,2,5-thiadiazolidin-2-yl)-5-fluoro-7-hydroxynaphthalen-2-yl)-2-((2-(1-(2,6-dioxopiperidin-3-yl)-3-methyl-2-oxo-2,3-dihydro-1H-benzo[d]imidazol-5-yl)-2-azaspiro[3.3]heptan-6-yl)oxy)acetamide (Degrader 96)

To a solution of N-(7-(benzyloxy)-6-(1,1-dioxido-4-oxo-1,2,5-thiadiazolidin-2-yl)-5-fluoronaphthalen-2-yl)-2-((2-(1-(2,6-bis(benzyloxy)pyridin-3-yl)-3-methyl-2-oxo-2,3-dihydro-1H-benzo[d]imidazol-5-yl)-2-azaspiro[3.3]heptan-6-yl)oxy)acetamide (3, 90 mg, 90.90 μmol, 1 eq) in DMF (2 mL) were added Pd/C (90.00 mg, 10% purity) and Pd(OH)₂/C (90 mg, 10% purity). The mixture was stirred under H₂ (15 Psi) at 30° C. for 16 hr under H₂. The mixture was filtered and concentrated under vacuum. The residue was purified by prep-HPLC (flow: 25 mL/min; gradient: from 15-45% MeCN in water (0.1% TFA) over 10 min; column: Phenomenex Synergi C18 150*25 mm*10 um) to afford N-(6-(1,1-dioxido-4-oxo-1,2,5-thiadiazolidin-2-yl)-5-fluoro-7-hydroxynaphthalen-2-yl)-2-((2-(1-(2,6-dioxopiperidin-3-yl)-3-methyl-2-oxo-2,3-dihydro-1H-benzo[d]imidazol-5-yl)-2-azaspiro[3.3]heptan-6-yl)oxy)acetamide (Degrader 96, 27.36 mg, 29.79 μmol, 33% yield, TFA salt) as a pink solid.

LCMS (ESI): m/z 722.2 [M+H]⁺

¹H NMR (400 MHz, DMSO-d6) δ=11.04 (s, 1H), 10.49-10.13 (m, 1H), 9.95 (s, 1H), 8.17 (s, 1H), 7.86 (d, J=8.9 Hz, 1H), 7.64-7.49 (m, 1H), 6.98 (s, 1H), 6.91 (d, J=8.3 Hz, 1H), 6.36 (s, 1H), 6.15 (br d, J=6.6 Hz, 1H), 5.27 (dd, J=5.3, 12.7 Hz, 1H), 4.34 (s, 2H), 4.11-4.06 (m, 1H), 4.02 (s, 2H), 3.82 (br d, J=8.3 Hz, 4H), 3.28 (s, 3H), 2.95-2.82 (m, 1H), 2.73-2.60 (m, 2H), 2.59-2.53 (m, 2H), 2.23 (ddd, J=2.1, 7.5, 10.1 Hz, 2H), 2.03-1.90 (m, 1H).

### N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)azepan-4-yl)acetamide (Degrader 97)

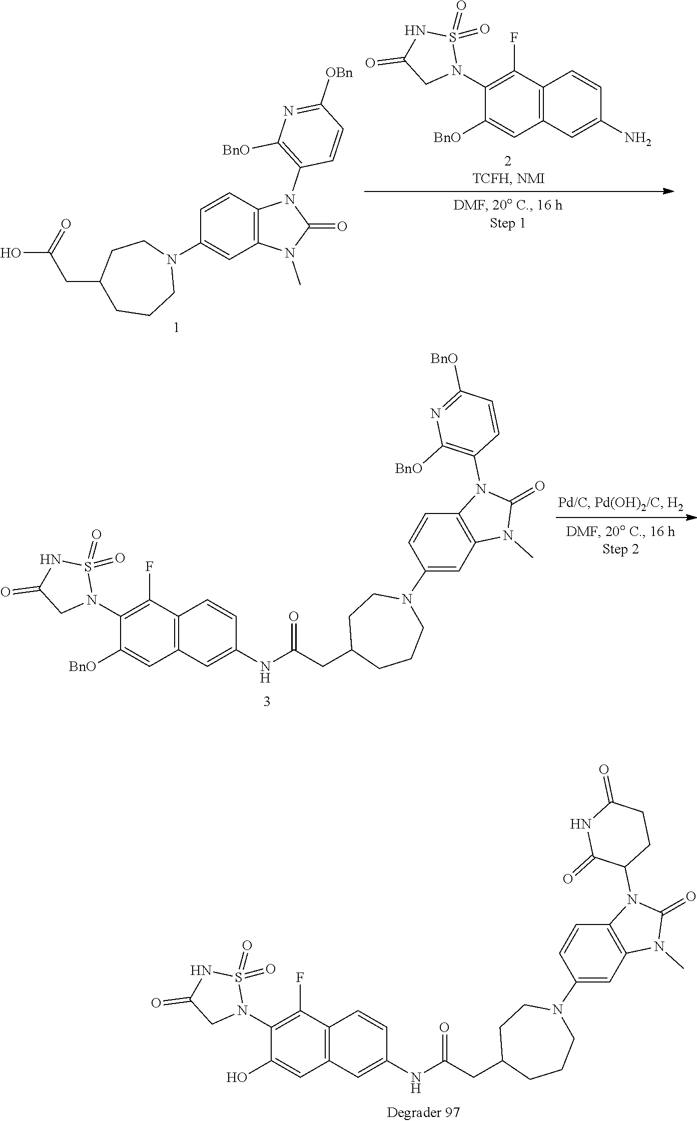

### Step 1: N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-methyl-2-oxo-2,3-dihydro-1H-benzo[d]imidazol-5-yl)azepan-4-yl)acetamide (3)

To a solution of 2-(1-(1-(2,6-bis(benzyloxy)pyridin-3-yl)-3-methyl-2-oxo-2,3-dihydro-1H-benzo[d]imidazol-5-yl)azepan-4-yl)acetic acid (1, 100 mg, 168.72 μmol), 5-(6-amino-3-(benzyloxy)-1-fluoronaphthalen-2-yl)-1,2,5-thiadiazolidin-3-one 1,1-dioxide (2, 88.05 mg, 219.34 μmol) in DMF (1 mL) was added NMI (69.26 mg, 0.84 mmol, 67.25 μL) and TCFH (118.35 mg, 0.42 mmol) at 0° C. The mixture was stirred at 30° C. for 16 h. The mixture was combined with another batch and poured into water (30 mL), then extracted with EA (20 mL*3). The combined organic layer was washed by water, brine, then dried over anhydrous Na₂SO₄, filtered and concentrated under vacuum. The residue was purified by prep-HPLC (flow: 25 mL/min; gradient: from 33%-63% water (0.05% ammonia hydroxide v/v)-ACN; column: Waters Xbridge 150×25 mm×5 um) to afford N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-methyl-2-oxo-2,3-dihydro-1H-benzo[d]imidazol-5-yl)azepan-4-yl)acetamide (3, 75 mg, 74.01 μmol, 44% yield) as a white solid.

LCMS (ESI): m/z 977.4 [M+H]⁺

### Step 2: N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)azepan-4-yl)acetamide (Degrader 97)

To a solution of N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-methyl-2-oxo-2,3-dihydro-1H-benzo[d]imidazol-5-yl)azepan-4-yl)acetamide (3, 70 mg, 71.72 μmol) in DMF (5 mL) was added Pd/C (50 mg, 10% purity) and Pd(OH)₂ (50 mg, 10% purity). The mixture was stirred at 25° C. for 16 h under H₂ (15 Psi). The reaction mixture was filtered and concentrated under reduced pressure to give a residue. The residue was purified by prep-HPLC (flow: 25 mL/min; gradient: from 37%-17% water (0.1% TFA)-ACN; column: 3_Phenomenex Luna C18 75×30 mm×3 um) to afford N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)azepan-4-yl)acetamide (Degrader 97, 2.22 mg, 2.67 μmol, 4% yield, TFA salt) as a blue solid.

LCMS (ESI): m/z 708.2 [M+H]⁺

¹H NMR (400 MHz, DMSO-d₆) δ 11.05 (s, 1H), 10.10 (s, 2H), 8.16 (s, 1H), 7.82 (d, J=8.9 Hz, 1H), 7.41 (br d, J=9.5 Hz, 1H), 7.01-6.82 (m, 2H), 6.67-6.33 (m, 2H), 5.37-5.18 (m, 1H), 4.29-4.18 (m, 2H), 3.57 (br d, J=5.0 Hz, 4H), 3.30 (br s, 3H), 2.90-2.88 (m, 1H), 2.70-2.68 (m, 2H), 2.33 (br d, J=1.9 Hz, 2H), 2.04-1.95 (m, 4H), 1.75-1.67 (m, 2H), 1.54-1.49 (m, 1H), 1.35-1.30 (m, 1H).

### (1R,2S)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)benzyl)cyclopropanecarboxamide (Degrader 98)

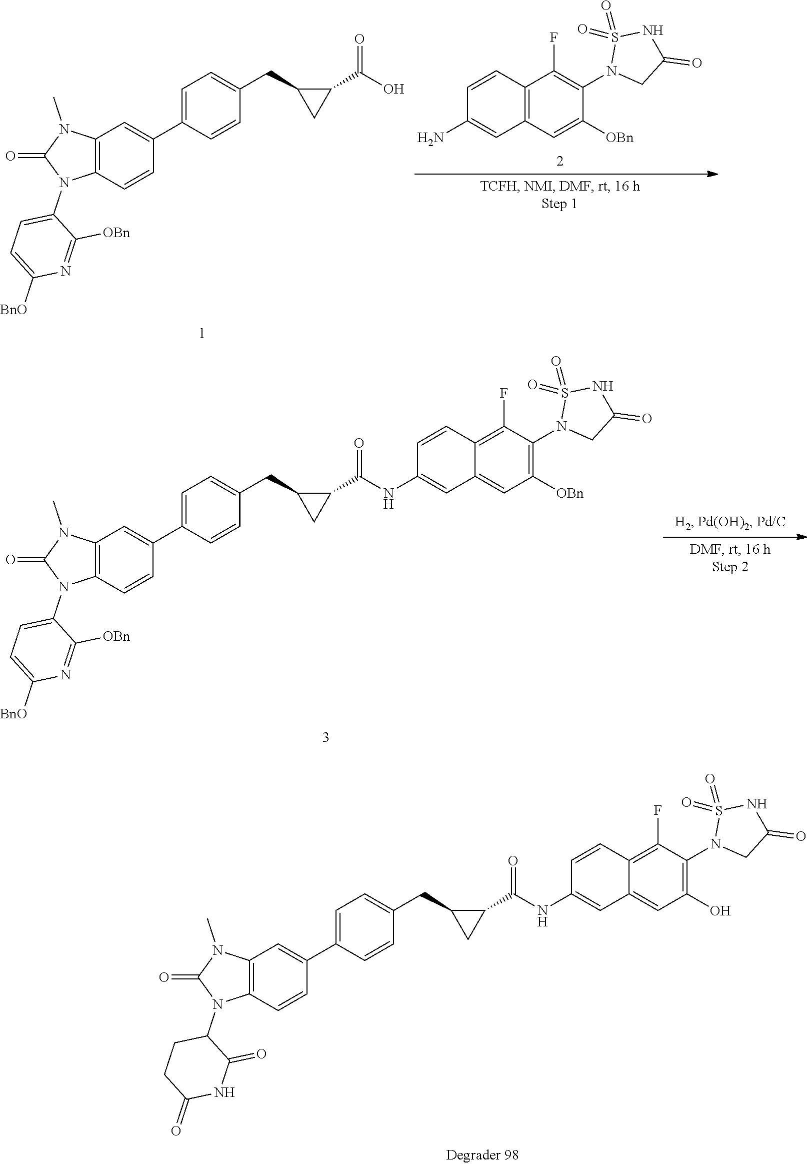 [SMILES: Cn1c(=O)n(-c2ccc(OCc3ccccc3)nc2OCc2ccccc2)c2ccc(-c3ccc(C[C@@H]4C[C@H]4C(=O)O)cc3)cc21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(-c3ccc(C[C@@H]4C[C@H]4C(=O)Nc4ccc5c(F)c(N6CC(=O)NS6(=O)=O)c(OCc6ccccc6)cc5c4)cc3)cc21 ; Cn1c(=O)n(C2CCC(=O)NC2=O)c2ccc(-c3ccc(C[C@@H]4C[C@H]4C(=O)Nc4ccc5c(F)c(N6CC(=O)NS6(=O)=O)c(O)cc5c4)cc3)cc21]

### Step 1: (1R,2S)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)benzyl)cyclopropanecarboxamide (3)

To a mixture of (1R,2S)-2-[[4-[1-(2,6-dibenzyloxy-3-pyridyl)-3-methyl-2-oxo-benzimidazol-5-yl]phenyl]methyl]cyclopropanecarboxylic acid (1, 150 mg, 228.06 μmol), 5-(6-amino-3-benzyloxy-1-fluoro-2-naphthyl)-1,1-dioxo-1,2,5-thiadiazolidin-3-one (2, 129.31 mg, 250.87 μmol, TFA salt) and NMI (93.62 mg, 1.14 mmol) in DMF (2.0 mL) was added TCFH (159.98 mg, 570.16 μmol) at 0° C. The mixture was stirred at 30° C. for 16 h. The mixture was concentrated in vacuum. The residue was purified by prep-HPLC (flow: 25 mL/min; gradient: from 35-65% MeCN in water (0.05% ammonia hydroxide v/v); column: Waters Xbridge 150*25 mm*5 um) and dried by lyophilization to afford (1R,2S)—N-[7-benzyloxy-5-fluoro-6-(1,1,4-trioxo-1,2,5-thiadiazolidin-2-yl)-2-naphthyl]-2-[[4-[1-(2,6-dibenzyloxy-3-pyridyl)-3-methyl-2-oxo-benzimidazol-5-yl]phenyl]methyl]cyclopropanecarboxamide (3, 35 mg, 34.92 μmol, 15% yield) as white solid.

LCMS (ESI): m/z 995.6[M+H]⁺

### Step 2: (1R,2S)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)benzyl)cyclopropanecarboxamide (Degrader 98)

To a solution of (1R,2S)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)benzyl)cyclopropanecarboxamide (3, 30 mg, 30.15 μmol) in DMF (0.5 mL) was added Pd/C (30 mg, 10% purity) and Pd(OH)₂/C (30 mg, 10% purity) under H₂ (15 PSI). The reaction mixture was stirred at 30° C. for 3 hrs under H₂ (15 Psi). The mixture was filtered and concentrated in vacuum. The residue was purified by prep-HPLC (flow: 25 mL/min; gradient: from 7-66% MeCN in water (0.1% TFA) over 7 min; column: 3_Phenomenex Luna C18 75*30 mm*3 um) and dried by lyophilization to afford (1R,2S)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)benzyl)cyclopropanecarboxamide (Degrader 98, 3.41 mg, 4.02 μmol, 13% yield, TFA salt) as white solid.

¹H NMR (400 MHz, DMSO) δ 11.11 (s, 1H), 10.41 (s, 1H), 9.70 (s, 1H), 8.13 (s, 1H), 7.80 (d, J=8.8 Hz, 1H), 7.64 (d, J=8.0 Hz, 2H), 7.49 (d, J=1.7 Hz, 1H), 7.44-7.30 (m, 4H), 7.17 (d, J=8.3 Hz, 1H), 6.90 (s, 1H), 5.39 (dd, J=12.8, 5.4 Hz, 1H), 4.06 (s, 2H), 3.40 (s, 2H), 3.30 (s, 3H), 2.89 (d, J=13.0 Hz, 1H), 2.77-2.59 (m, 2H), 2.09-1.99 (m, 1H), 1.88-1.78 (m, 1H), 1.64-1.52 (m, 1H), 1.16-1.06 (m, 1H), 0.96-0.87 (m, 1H).

LCMS (ES+): m/z 727.2 [M+H]⁺

### N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)-4-methoxypiperidin-4-yl)acetamide (Degrader 99)

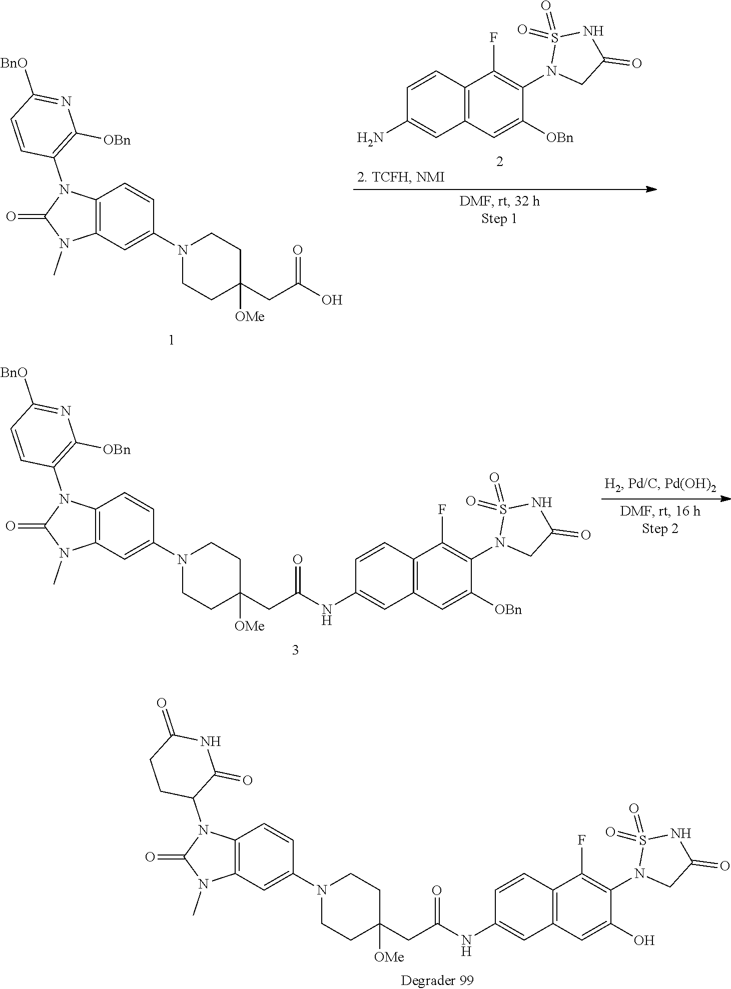 [SMILES: COC1(CC(=O)O)CCN(c2ccc3c(c2)n(C)c(=O)n3-c2ccc(OCc3ccccc3)nc2OCc2ccccc2)CC1 ; Nc1ccc2c(F)c(N3CC(=O)NS3(=O)=O)c(OCc3ccccc3)cc2c1 ; COC1(CC(=O)Nc2ccc3c(F)c(N4CC(=O)NS4(=O)=O)c(OCc4ccccc4)cc3c2)CCN(c2ccc3c(c2)n(C)c(=O)n3-c2ccc(OCc3ccccc3)nc2OCc2ccccc2)CC1 ; COC1(CC(=O)Nc2ccc3c(F)c(N4CC(=O)NS4(=O)=O)c(O)cc3c2)CCN(c2ccc3c(c2)n(C)c(=O)n3C2CCC(=O)NC2=O)CC1]

### Step 1: N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-methyl-2-oxo-2,3-dihydro-1H-benzo[d]imidazol-5-yl)-4-methoxypiperidin-4-yl)acetamide (3)

N-ethyl-N-isopropyl-propan-2-amine (256.63 mg, 1.99 mmol, 345.86 μL) in DMF (1.0 mL) was added a mixture of 2-(1-(1-(2,6-bis(benzyloxy)pyridin-3-yl)-3-methyl-2-oxo-2,3-dihydro-1H-benzo[d]imidazol-5-yl)-4-methoxypiperidin-4-yl)acetic acid (1, 130 mg, 198.56 μmol, formic acid salt) and 5-(6-amino-3-(benzyloxy)-1-fluoronaphthalen-2-yl)-1,2,5-thiadiazolidin-3-one 1,1-dioxide (2, 102.35 mg, 198.56 μmol, 061) at 0° C. Then T₃P (631.79 mg, 992.81 μmol, 50% purity) was added at 0° C. The reaction mixture was stirred at 50° C. for 12 hrs. The mixture was filtered and concentrated in vacuum. The residue was purified by prep-HPLC (flow: 25 mL/min; gradient: from 23-53% MeCN in water (0.05% ammonia hydroxide) over 12 min; column: Waters Xbridge 150*25 mm*5 um) to afford N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-methyl-2-oxo-2,3-dihydro-1H-benzo[d]imidazol-5-yl)-4-methoxypiperidin-4-yl)acetamide (3, 100 mg, 100.80 μmol, 51% yield) as yellow solid.

LCMS (ESI): m/z 992.6 [M+H]⁺

### Step 2: N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)-4-methoxypiperidin-4-yl)acetamide (Degrader 99)

To a solution of N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-methyl-2-oxo-2,3-dihydro-1H-benzo[d]imidazol-5-yl)-4-methoxypiperidin-4-yl)acetamide (3, 90 mg, 90.72 μmol) in DMF (2 mL) was added Pd/C (90 mg, 10% purity) and Pd(OH)₂/C (90 mg, 10% purity) under H₂. The reaction mixture stirred at 30° C. for 3 hrs under H₂ (15 Psi). The mixture was filtered and concentrated in vacuum. The residue was purified by prep-HPLC (flow: 25 mL/min; gradient: from 10-40% MeCN in water (0.1% TFA) over 7 min; column: 3_Phenomenex Luna C18 75*30 mm*3 um) and lyophilization to afford N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)-4-methoxypiperidin-4-yl)acetamide (Degrader 99, 15.89 mg, 18.78 μmol, 21% yield, TFA salt) as white solid.

¹H NMR (400 MHz, DMSO-d6) δ=11.12 (s, 1H), 10.26 (br s, 1H), 10.14-9.90 (m, 1H), 8.20 (s, 1H), 7.87 (d, J=9.0 Hz, 1H), 7.45 (br d, J=9.0 Hz, 1H), 7.38-6.93 (m, 4H), 5.37 (br dd, J=4.7, 11.4 Hz, 1H), 4.23 (s, 2H), 3.45 (br d, J=6.9 Hz, 5H), 3.37 (s, 3H), 3.30 (s, 3H), 2.94-2.85 (m, 1H), 2.75 (br d, J=4.5 Hz, 1H), 2.68-2.59 (m, 2H), 2.19-1.97 (m, 5H).

LCMS (ES+): m/z 724.2 [M+H]⁺.

### 2-[4-[1-(2,6-dioxo-3-piperidyl)-3-methyl-indazol-5-yl]-1-piperidyl]-N-[5-fluoro-7-hydroxy-6-(1,1,4-trioxo-1,2,5-thiadiazolidin-2-yl)-2-naphthyl]acetamide (Degrader 100)

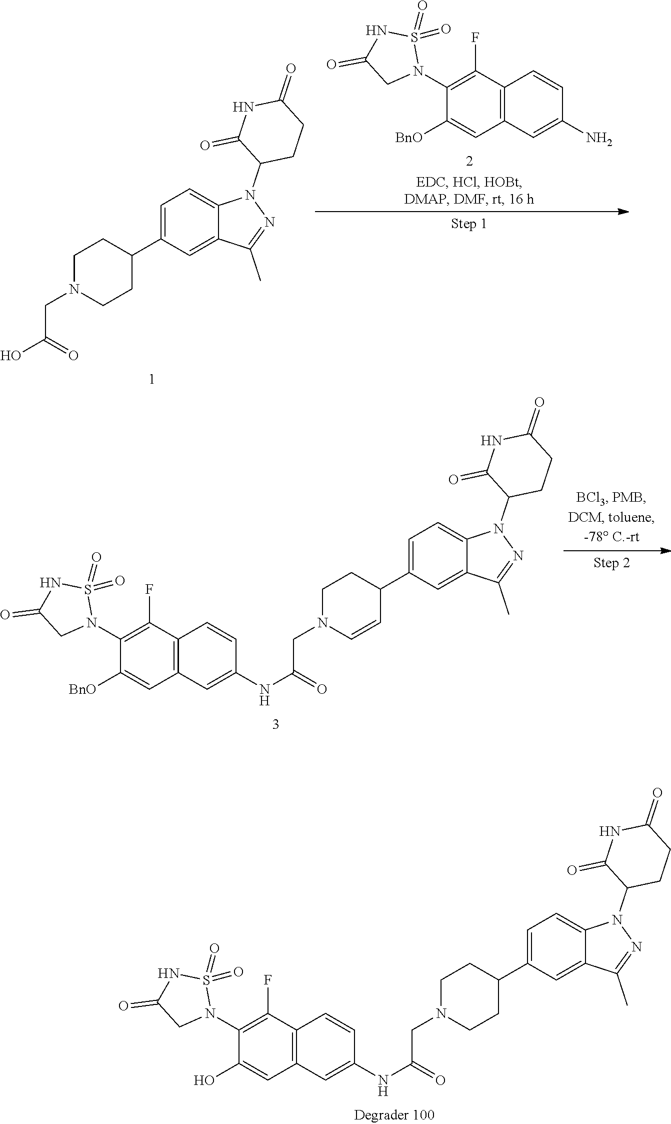

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-indazol-5-yl]-1-piperidyl]acetamide (3)

Into a 20 mL vial containing a well-stirred solution of 5-(6-amino-3-benzyloxy-1-fluoro-2-naphthyl)-1,1-dioxo-1,2,5-thiadiazolidin-3-one (2, 120 mg, 227.95 μmol, TFA salt) in DMF (2 mL) were added 2-[4-[1-(2,6-dioxo-3-piperidyl)-3-methyl-indazol-5-yl]-1-piperidyl]acetic acid (1, 97.37 mg, 175.80 μmol, TFA salt) and EDC hydrochloride (131.09 mg, 683.84 μmol), 4-dimethylaminopyridine (167.09 mg, 1.37 mmol) and hydroxybenzotriazole (61.60 mg, 455.90 μmol). The reaction mixture was stirred at room temperature. After 16 h, the solvent was removed under reduced pressure. The concentrate was treated with 1.5 N HCl and The precipitate was filtered, washed with water and dried under vacuum to get N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-indazol-5-yl]-1-piperidyl]acetamide (3, 140 mg, 113.05 μmol, 50% yield) crude compound as an brown solid.

LCMS (ES+): m/z 768.0 [M+H]⁺

### Step 2: 2-[4-[1-(2,6-dioxo-3-piperidyl)-3-methyl-indazol-5-yl]-1-piperidyl]-N-[5-fluoro-7-hydroxy-6-(1,1,4-trioxo-1,2,5-thiadiazolidin-2-yl)-2-naphthyl]acetamide (Degrader 100)

Into a 50 mL round bottom flask containing a well-stirred solution of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-indazol-5-yl]-1-piperidyl]acetamide (3, 140 mg, 113.05 μmol) in anhydrous DCM (2 mL) toluene (2 mL) were added pentamethyl benzene (83.79 mg, 565.23 μmol) and a 1.0 M solution of boron trichloride solution in DCM (2.26 mmol, 2.26 mL) at −78° C. The reaction was brought to room temperature and stirred for 3 h. The reaction mixture was cooled to −78° C. and quenched slowly with 5% methanol in dichloromethane (1 mL). The reaction mixture was concentrated under reduced pressure and the residue was washed with MTBE (10 mL), filtered and dried. The crude compound was purified by reverse phase prep HPLC [Purification method: Column: XBridge C18 (150×19) mm, 5 micron; Mobile phase A: 0.1% formic acid in water; Mobile phase B: MeCN] to get 2-[4-[1-(2,6-dioxo-3-piperidyl)-3-methyl-indazol-5-yl]-1-piperidyl]-N-[5-fluoro-7-hydroxy-6-(1,1,4-trioxo-1,2,5-thiadiazolidin-2-yl)-2-naphthyl]acetamide (Degrader 100, 10 mg, 13.33 μmol, 12% yield, formic acid salt) as an off-white solid.

LCMS (ES+): m/z 678.2 [M+H]⁺

¹H NMR (400 MHz, DMSO) δ 11.06 (s, 1H), 10.78 (s, 1H), 9.82 (s, 1H), 8.14-8.11 (m, 1H), 7.89 (d, J=9.0 Hz, 1H), 7.58-7.45 (m, 3H), 7.34 (d, J=8.7 Hz, 1H), 7.00 (s, 1H), 5.72 (dd, J=11.8, 5.1 Hz, 1H), 4.32-4.20 (m, 1H), 4.08 (s, 2H), 3.79-3.62 (m, 1H), 2.96 (s, 1H), 2.91-2.68 (m, 4H), 2.26-2.18 (m, 1H), 2.16-1.94 (m, 4H). Additional protons under solvent peak.

### 2-[1-[1-(2,6-dioxo-3-piperidyl)-3-methyl-indazol-5-yl]-4-piperidyl]-N-[5-fluoro-7-hydroxy-6-(1,1,4-trioxo-1,2,5-thiadiazolidin-2-yl)-2-naphthyl]acetamide (Degrader 101)

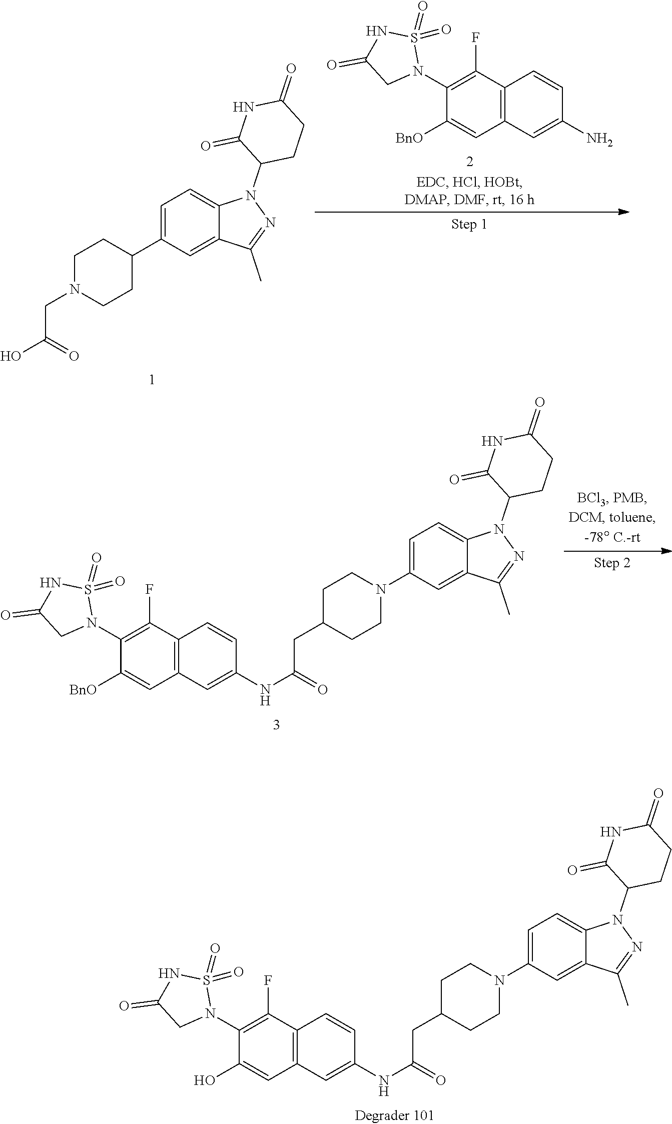

### Step 1: N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-indazol-5-yl]-4-piperidyl]acetamide (3)

Into a 8 mL vial containing a well-stirred solution of 5-(6-amino-3-benzyloxy-1-fluoro-2-naphthyl)-1,1-dioxo-1,2,5-thiadiazolidin-3-one (2, 40 mg, 75.28 μmol, TFA salt) in DMF (1.5 mL) were added 2-[1-[1-(2,6-dioxo-3-piperidyl)-3-methyl-indazol-5-yl]-4-piperidyl]acetic acid (1, 39.64 mg, 58.06 μmol, TFA salt), N-(3-dimethylaminopropyl)-N′-ethylcarbodiimide hydrochloride (43.29 mg, 225.83 μmol), hydroxybenzotriazole (20.34 mg, 150.55 μmol) and DMAP (55.18 mg, 451.66 μmol). The reaction mixture was stirred at room temperature for 16 h. After completion of the reaction, the solvent was removed under reduced pressure and treated with 1.5N HCl. The precipitate was filtered, washed with water and dried under vacuum to get N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-indazol-5-yl]-4-piperidyl]acetamide (3, 70 mg, 40.11 μmol, 53% yield) crude compound as an brown solid.

UPLC-MS (ES+): m/z 768.0 [M+H]⁺

### Step 2: 2-[1-[1-(2,6-dioxo-3-piperidyl)-3-methyl-indazol-5-yl]-4-piperidyl]-N-[5-fluoro-7-hydroxy-6-(1,1,4-trioxo-1,2,5-thiadiazolidin-2-yl)-2-naphthyl]acetamide (Degrader 101)

Into a 25 mL round bottom flask containing a well-stirred solution of N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-indazol-5-yl]-4-piperidyl]acetamide (3, 60 mg, 35.16 μmol) and pentamethylbenzene (26.06 mg, 175.82 μmol) in anhydrous DCM (1 mL) and toluene (1 mL) was added a 1.0 M solution of boron trichloride solution 1.0 M in DCM (703.29 μmol, 0.70 mL) at −78° C. The reaction mixture was stirred at room temperature. After 3 h, the reaction mixture was cooled to −78° C. and quenched slowly with 5% methanol in dichloromethane (0.5 mL). The reaction mixture was concentrated under reduced pressure at 30° C. The residue was triturated with diethylether (10 mL), filtered and dried. The crude compound was purified by reverse phase column chromatography [Silicycle C18 column; Mobile phase A: 0.1% formic acid in water; Mobile phase B: MeCN] to get 2-[1-[1-(2,6-dioxo-3-piperidyl)-3-methyl-indazol-5-yl]-4-piperidyl]-N-[5-fluoro-7-hydroxy-6-(1,1,4-trioxo-1,2,5-thiadiazolidin-2-yl)-2-naphthyl]acetamide (Degrader 101, 5 mg, 6.31 μmol, 18% yield, Formic acid salt) as an off-white solid.

LCMS (ES+): m/z 678.0 [M+H]⁺

¹H NMR (400 MHz, DMSO) δ 11.03 (s, 1H), 10.16 (s, 1H), 9.77 (s, 1H), 8.17 (s, 1H), 7.82 (d, J=9.0 Hz, 1H), 7.51-7.37 (m, 2H), 7.24 (s, 1H), 7.09 (s, 1H), 6.94 (s, 1H), 5.70-5.59 (m, 1H), 4.08 (s, 2H), 3.63-3.55 (m, 2H), 2.90-2.79 (m, 1H), 2.75-2.70 (m, 2H), 2.43 (s, 3H), 2.38 (d, J=6.7 Hz, 1H), 2.27-2.16 (m, 1H), 2.08 (d, J=4.3 Hz, 1H), 2.04-1.93 (m, 1H), 1.91-1.77 (m, 2H), 1.60-1.38 (m, 2H).

### N-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)-3,3-difluoropiperidin-1-yl)cyclobutanecarboxamide (Degrader 102)

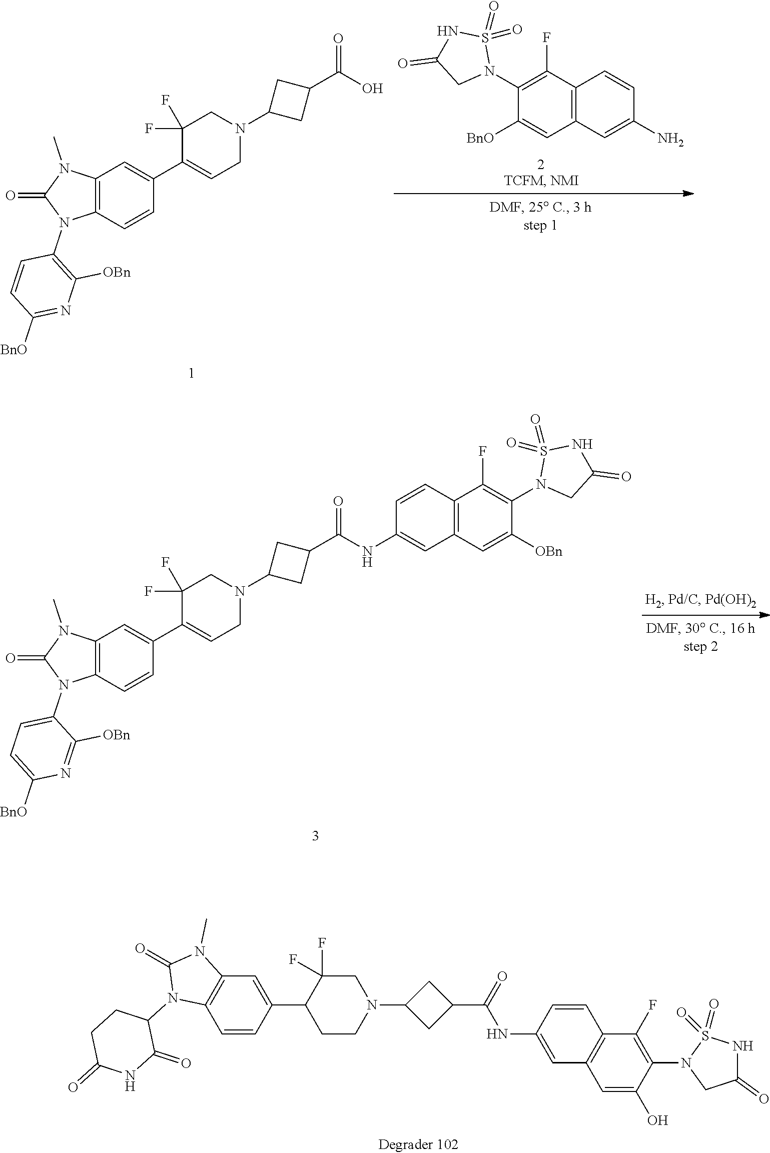

### Step 1: N-(7-(benzyloxy)-6-(1,1-dioxido-4-oxo-1,2,5-thiadiazolidin-2-yl)-5-fluoronaphthalen-2-yl)-3-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)cyclobutanecarboxamide (3)

To a solution of 3-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)cyclobutanecarboxylic acid (1, 130 mg, 199.18 μmol) and 5-(6-amino-3-(benzyloxy)-1-fluoronaphthalen-2-yl)-1,2,5-thiadiazolidin-3-one 1,1-dioxide (2, 102.66 mg, 199.18 μmol, TFA salt), 1-methylimidazole (81.77 mg, 995.89 μmol, 79.38 μL) in DMF (2.5 mL) was added [chloro(dimethylamino)methylene]-dimethyl-ammonium; hexafluorophosphate (139.71 mg, 497.94 μmol) at 0° C. The mixture was stirred at 30° C. for 12 h. the mixture was purified by Prep-HPLC (0.1% NH₄HCO₃) to obtain N-(7-(benzyloxy)-6-(1,1-dioxido-4-oxo-1,2,5-thiadiazolidin-2-yl)-5-fluoronaphthalen-2-yl)-3-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)cyclobutanecarboxamide (3, 100 mg, 91.69 μmol, 46% yield) LCMS (ESI): m/z 1036.0 [M+H]⁺

### Step 2: N-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)-3,3-difluoropiperidin-1-yl)cyclobutanecarboxamide (Degrader 102)

To a solution of N-(7-(benzyloxy)-6-(1,1-dioxido-4-oxo-1,2,5-thiadiazolidin-2-yl)-5-fluoronaphthalen-2-yl)-3-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)cyclobutanecarboxamide (3, 60 mg, 57.91 μmol) in DMF (4 mL) were added Pd/C (60 mg, 49.40 μmol, 10% purity) and Pd(OH)₂/C (60 mg, 57.91 μmol, 10% purity) under N₂ atmosphere. The suspension was degassed and purged with H₂ 3 times. The mixture was stirred under H₂ (15 psi) at 30° C. for 16 h. The mixture was filtered and concentrated in vacuum. The residue was purified by prep-HPLC(Column: 3_Phenomenex Luna C18 75*30 mm*3 um, water (0.1% TFA)-ACN, Flow Rate (25 ml/min)) to obtain N-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)-3,3-difluoropiperidin-1-yl)cyclobutanecarboxamide (Degrader 102, 23.3 mg, 25.84 μmol, 45% yield, TFA salt) as pink solid.

LCMS (ESI): m/z 770.0 [M+H]+

1H NMR (400 MHz, DMSO-d6) δ=11.11 (s, 1H), 10.84 (br s, 1H), 10.12-9.93 (m, 1H), 9.89-9.64 (m, 1H), 8.15 (s, 1H), 7.93 (d, J=8.9 Hz, 1H), 7.50 (dd, J=1.6, 9.0 Hz, 1H), 7.16-6.84 (m, 4H), 5.37 (br dd, J=5.3, 12.7 Hz, 1H), 4.27-3.98 (m, 3H), 3.89-3.71 (m, 1H), 3.36 (s, 3H), 3.24-3.11 (m, 1H), 3.05-2.83 (m, 2H), 2.79-2.60 (m, 4H), 2.23-1.90 (m, 5H), 1.66 (br d, J=6.8 Hz, 2H)

### (2R)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)propanamide (Degrader 103)

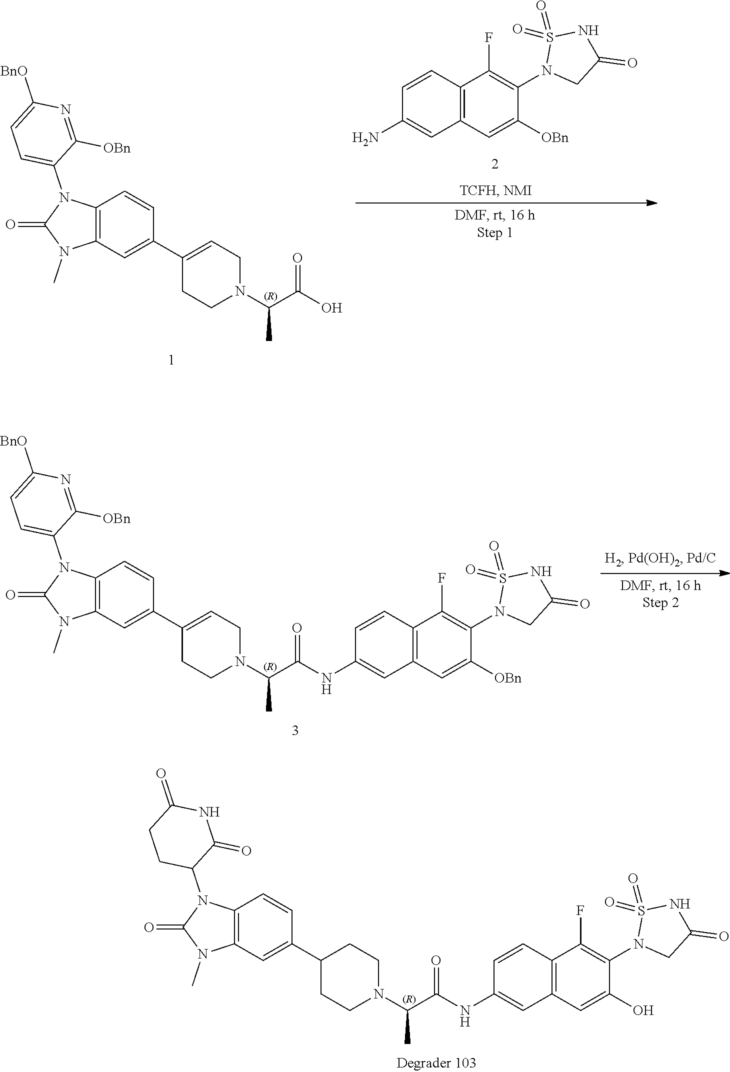

Note: Configurations are arbitrarily assigned.

### Step 1: (R)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanamide (3)

To a mixture of 5-(6-amino-3-(benzyloxy)-1-fluoronaphthalen-2-yl)-1,2,5-thiadiazolidin-3-one 1,1-dioxide (2, 87.26 mg, 169.30 μmol, TFA salt), (R)-2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanoic acid (1, 100.00 mg, 169.30 μmol) and 1-methylimidazole (69.50 mg, 846.50 μmol, 67.48 μL) in DMF (2 mL) was added [chloro(dimethylamino)methylene]-dimethyl-ammonium; hexafluorophosphate (118.76 mg, 423.25 μmol) at 0° C. The mixture was stirred at 30° C. for 16 h. The residue was purified by revered phase (0.1% NH₄HCO₃) to obtain (R)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanamide (3, 80 mg, 78.02 μmol, 46% yield) as yellow solid LCMS (ESI): m/z 975.0 [M+H]⁺

### Step 2: (2R)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)propanamide (Degrader 103)

To a solution of (R)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanamide (3.40 mg, 41.07 μmol) in DMF (2 mL) were added Pd(OH)₂/C (40 mg, 41.07 μmol, 10% purity) and Pd/C (40 mg, 32.93 μmol, 10% purity) under N₂ atmosphere. The suspension was degassed and purged with H₂ 3 times. The mixture was stirred under H₂ (15 psi) at 30° C. for 16 h. The mixture was filtered and concentrated in vacuum. The residue was purified by prep-HPLC(Column: 3_Phenomenex Luna C18 75*30 mm*3 um, water (0.1% TFA)-ACN, FlowRate (25 ml/min)) to obtain (2R)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)propanamide (Degrader 103, 10.95 mg, 13.33 μmol, 32% yield, TFA salt) as yellow solid

LCMS (ESI): m/z 708.1 [M+H]⁺

¹H NMR (400 MHz, DMSO) δ 11.10 (s, 1H), 10.83 (s, 1H), 10.01 (s, 1H), 9.75 (s, 1H), 8.14 (s, 1H), 7.92 (d, J=9.0 Hz, 1H), 7.50 (dd, J=9.0, 2.0 Hz, 1H), 7.08 (d, J=8.2 Hz, 1H), 7.05-7.00 (m, 2H), 6.96-6.90 (m, 1H), 5.36 (dd, J=12.7, 5.4 Hz, 1H), 4.19-4.14 (m, 2H), 4.14-4.04 (m, 1H), 3.81-3.72 (m, 1H), 3.62-3.53 (m, 2H), 3.35 (s, 3H), 3.23-3.12 (m, 1H), 2.98-2.85 (m, 2H), 2.76-2.56 (m, 2H), 2.19-1.96 (m, 5H), 1.65 (d, J=6.9 Hz, 3H).

### (2S)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)propanamide (Degrader 104)

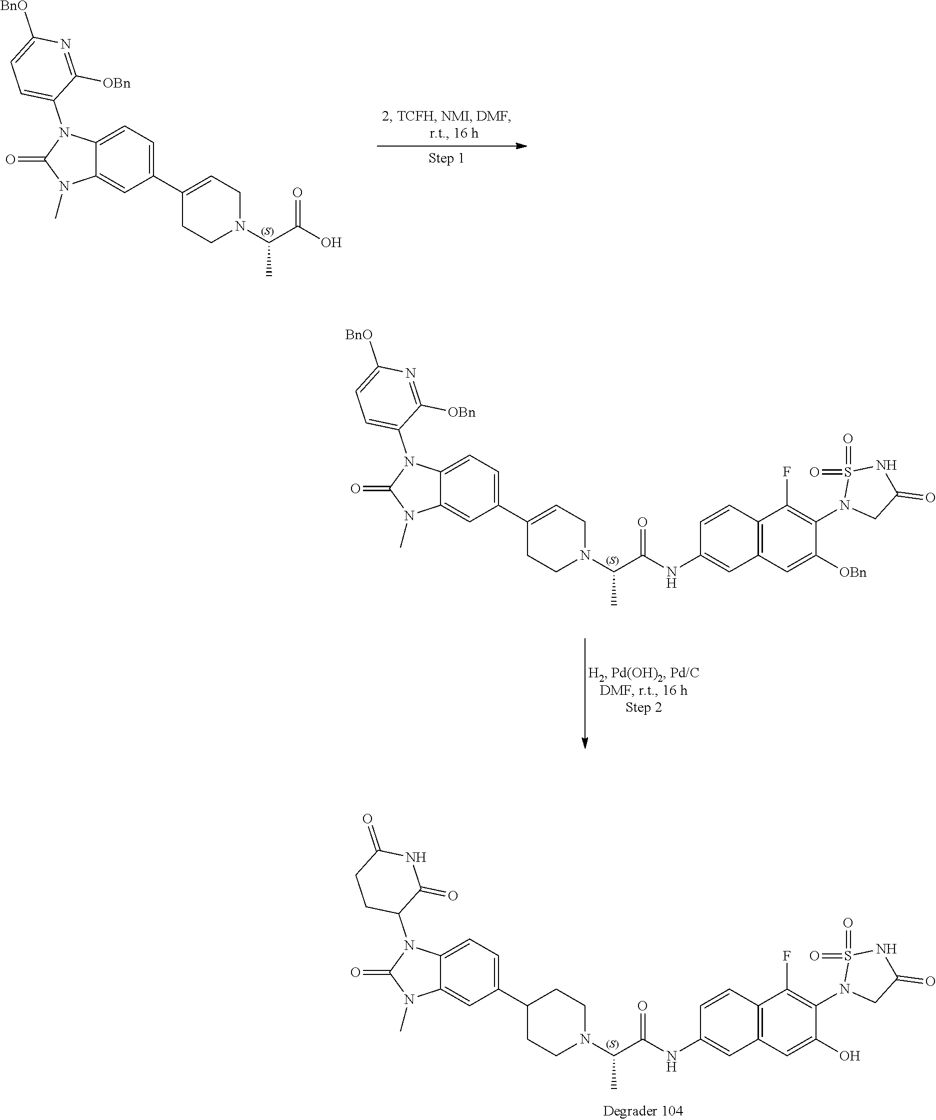

### Step 1: (S)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanamide (3)

To a mixture of (S)-2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanoic acid (1, 95 mg, 160.84 μmol), 5-(6-amino-3-(benzyloxy)-1-fluoronaphthalen-2-yl)-1,2,5-thiadiazolidin-3-one 1,1-dioxide (2, 82.90 mg, 160.84 μmol, 061) and 1-methylimidazole (66.03 mg, 804.18 μmol, 64.10 μL) in DMF (2 mL) was added TCFH (112.82 mg, 402.09 μmol) at 0° C. The mixture was stirred at 30° C. for 16 h. The mixture was purified by prep-HPLC (flow: 25 mL/min; gradient: from 47-77% MeCN in water (0.1% FA); column: Waters Xbridge 150×25 mm×5 um) to afford (S)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanamide (3, 120 mg, 117.04 μmol, 73% yield) as yellow solid.

LCMS (ESI): m/z 974.8 [M+H]⁺

### Step 2: (2S)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)propanamide (Degrader 104)

To a solution of (S)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-5,6-dihydropyridin-1 (2H)-yl)propanamide (3, 80 mg, 82.13 μmol) in DMF (4 mL) was added Pd/C (80 mg, 65.87 μmol, 10% purity) and Pd(OH)₂/C (80 mg, 82.13 μmol, 10% purity) under N₂ atmosphere. The suspension was degassed and purged with H₂ 3 times. The mixture was stirred under H₂ (15 psi) at 30° C. for 16 h. The reaction mixture was filtered and concentrated in vacuum. The residue was purified by prep-HPLC (flow: 25 mL/min; gradient: from 32-52% MeCN in water (0.1% TFA) over 9 min; column: 3_Phenomenex Luna C18 75×30 mm×3 μm) to afford (2S)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)propanamide (Degrader 104, 30.72 mg, 37.38 μmol, 46% yield, TFA salt) as a pink solid.

LCMS (ESI): m/z 708.0 [M+H]⁺

¹H NMR (400 MHz, DMSO-d₆) δ=11.11 (s, 1H), 10.83 (br s, 1H), 10.04-9.62 (m, 2H), 8.15 (s, 1H), 7.93 (d, J=8.8 Hz, 1H), 7.50 (dd, J=1.6, 9.0 Hz, 1H), 7.20-6.88 (m, 4H), 5.47-5.26 (m, 1H), 4.14 (br s, 3H), 3.83-3.70 (m, 1H), 3.24-3.14 (m, 1H), 3.02-2.86 (m, 3H), 2.80-2.61 (m, 5H), 2.18-1.93 (m, 5H), 1.66 (br d, J=6.2 Hz, 3H)

### (2S)—N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)propanamide (Degrader 105)

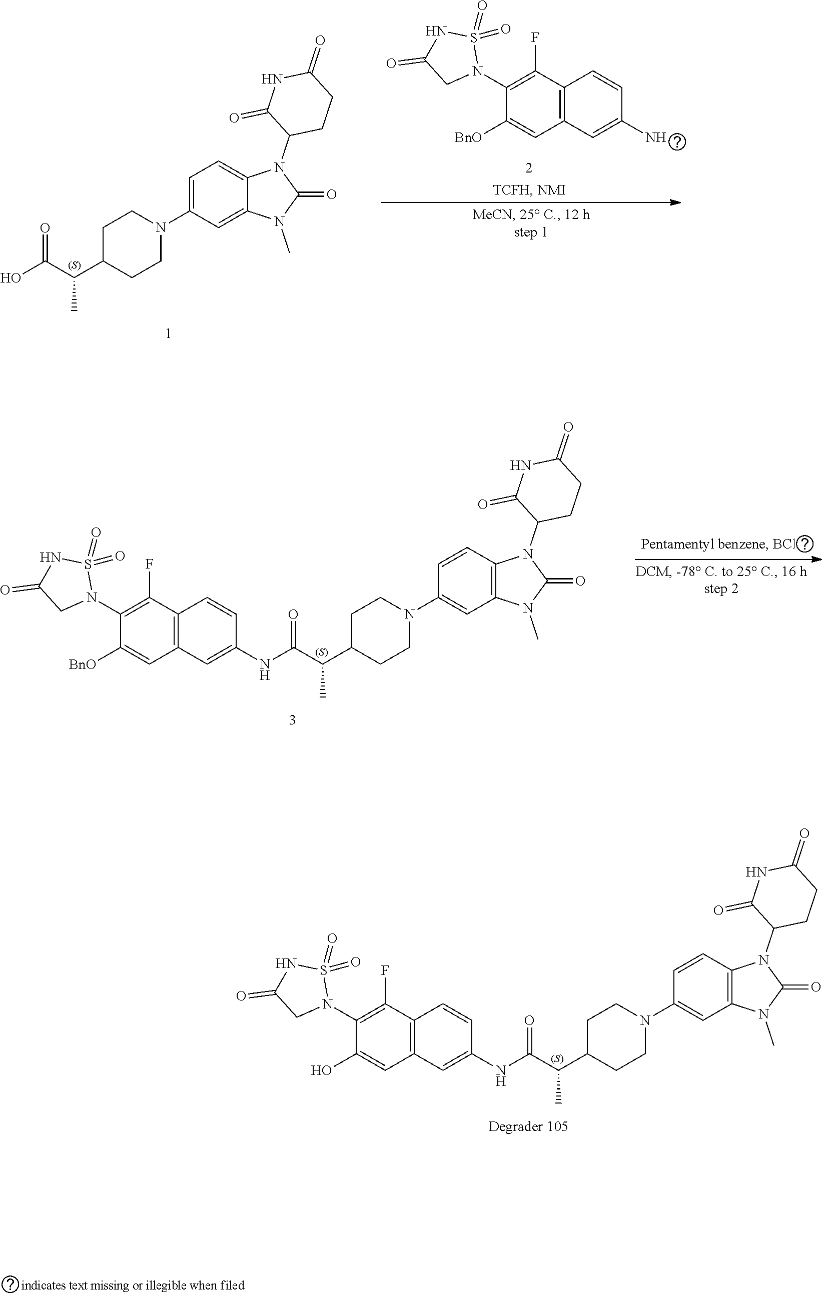

Note: Configurations are arbitrarily assigned.

### Step 1: (2S)—N-(7-(benzyloxy)-6-(1,1-dioxido-4-oxo-1,2,5-thiadiazolidin-2-yl)-5-fluoronaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)propanamide (3)

To a solution of (2S)-2-(1-(1-(2,6-dioxopiperidin-3-yl)-3-methyl-2-oxo-2,3-dihydro-1H-benzo[d]imidazol-5-yl)piperidin-4-yl)propanoic acid (1, 130 mg, 313.67 μmol), 5-(6-amino-3-(benzyloxy)-1-fluoronaphthalen-2-yl)-1,2,5-thiadiazolidin-3-one 1,1-dioxide (2, 151.09 mg, 376.40 μmol) in ACN (3 mL) was added [chloro (dime thylamino)methylene]-dimethyl-ammonium; hexafluorophosphate (220.02 mg, 784.16 μmol) and 1-Methylimidazole (128.77 mg, 1.57 mmol, 125.02 μL). The mixture was stirred at 25° C. for 12 h. The crude product was purified by Prep-HPLC (flow: 25 mL/min; gradient: from 22-52% MeCN in water (0.225% FA)-ACNUnisil 3-100 C18 Ultra 150*50 mm*3 um to afford (2S)—N-(7-(benz yloxy)-6-(1,1-dioxido-4-oxo-1,2,5-thiadiazolidin-2-yl)-5-fluoronaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)propenamide (3, 130 mg, 162.94 μmol, 52% yield) as white solid. The stereo center of this compound was assigned arbitrarily.

LCMS (ESI): m/z 798.1 [M+H]⁺

### Step 2: (2S)—N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)propanamide (Degrader 105)

To a solution of (2S)—N-(7-(benzyloxy)-6-(1,1-dioxido-4-oxo-1,2,5-thiadiazolidin-2-yl)-5-fluoronaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)propanamide (3, 0.13 g, 162.94 μmol) in DMF (5 mL) was added Pd(OH)₂/C (130 mg, 162.94 μmol, 10% purity). The mixture was stirred at 25° C. under H₂ (15 psi) for 3 h. The mixture was filtered and the filter was concentrated under vacuum. The crude product was purified by Prep-HPLC (flow: 25 mL/min; gradient: from 16-46% water (0.1% TFA)-ACN, column: Phenomenex Synergi C18 150*25 mm*10 um to afford ((2S)—N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)propanamide (Degrader 105, 16.73 mg, 20.36 μmol, 12% yield, TFA salt) as pink solid. The stereocenters of this compound was assigned arbitrarily.

LCMS (ESI): m/z 708.5 [M+H]+

¹H NMR (400 MHz, DMSO-d6) δ=11.16-11.06 (m, 1H), 10.30-9.90 (m, 2H), 8.21 (s, 1H), 7.85 (d, J=8.9 Hz, 1H), 7.55-6.70 (m, 5H), 5.56-5.23 (m, 1H), 4.22 (br s, 2H), 3.74-3.60 (m, 4H), 3.35 (s, 3H), 2.95-2.83 (m, 1H), 2.76-2.62 (m, 2H), 2.36-2.30 (m, 1H), 2.05-1.95 (m, 2H), 1.93-1.78 (m, 2H), 1.74-1.52 (m, 2H), 1.18 (br d, J=6.6 Hz, 3H).

### (2R)—N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)propanamide (Degrader 106)

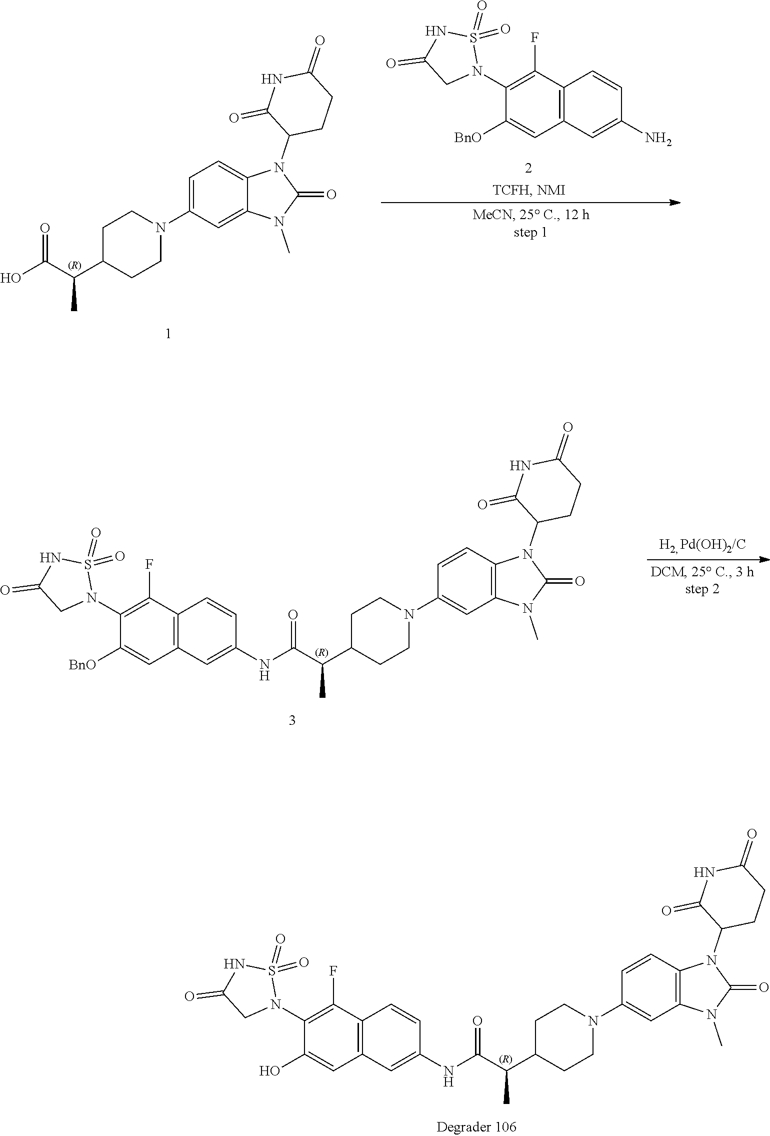

Note: Configurations are arbitrarily assigned.

### Step 1: (2R)—N-(7-(benzyloxy)-6-(1,1-dioxido-4-oxo-1,2,5-thiadiazolidin-2-yl)-5-fluoronaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)propanamide (3)

To a solution of (2R)-2-(1-(1-(2,6-dioxo piperidin-3-yl)-3-methyl-2-oxo-2,3-dihydro-1H-benzo[d]imidazol-5-yl)piperidin-4-yl)propanoic acid (1,130 mg, 313.67 μmol), 5-(6-amino-3-(benzyloxy)-1-fluoronaphthalen-2-yl)-1,2,5-thiadiazolidin-3-one 1,1-dioxide (2, 151.09 mg, 376.40 μmol) in ACN (3 mL) was added [chloro (dime thylamino)methylene]-dimethyl-ammonium; hexafluorophosphate (784.16 μmol) and 1-Methylimidazole (128.77 mg, 1.57 mmol, 125.02 uL). The mixture was stirred at 25° C. for 12 h. The crude product was purified by Prep-HPLC (flow: 25 mL/min; gradient: from 21-51% MeCN in water (0.225% FA)-ACN, column: Phenomenex luna C18 150*25 mm*10 um to afford (2R)—N-(7-(benzyloxy)-6-(1,1-dioxido-4-oxo-1,2,5-thiadiazolidin-2-yl)-5-fluoronaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)propanamide (3, 0.150 g, 188.01 μmol, 60% yield) as white solid. The stereo center of this compound was assigned arbitrarily.

LCMS (ESI): m/z 798.1 [M+H]⁺

### Step 2: (2R)—N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)propanamide (Degrader 106)

To a solution of (2R)—N-(7-(benzyloxy)-6-(1,1-dioxido-4-oxo-1,2,5-thiadiazolidin-2-yl)-5-fluoronaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)propanamide (3, 150 mg, 188.01 μmol) in DMF (5 mL) was added Pd(OH)₂/C (150 mg, 188.01 μmol, 10% purity). The mixture was stirred at 25° C. under H₂ (15 Psi) for 3 h. The mixture combined with another batch and filtered, the filter was concentrated under vacuum. The crude product was purified by Prep-HPLC (flow: 25 mL/min; gradient: from 16-46% water (0.1% TFA)-ACN, column: Phenomenex Synergi C18 150*25 mm*10 um to afford (2R)—N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)propanamide (Degrader 106, 23.64 mg, 28.77 μmol, 15% yield, TFA salt) was obtained as pink solid. The stereocenters of this compound was assigned arbitrarily.

LCMS (ESI): m/z 708.5 [M+H]⁺

¹H NMR (400 MHz, DMSO-d6) δ=11.13 (br s, 1H), 10.59-9.98 (m, 2H), 8.22 (s, 1H), 7.87 (br d, J=8.8 Hz, 1H), 7.76-6.85 (m, 5H), 5.48-5.33 (m, 1H), 4.40-4.24 (m, 2H), 3.64 (br s, 4H), 3.29 (br s, 3H), 2.98-2.84 (m, 1H), 2.78-2.64 (m, 2H), 2.40-2.30 (m, 1H), 2.15-1.99 (m, 2H), 1.98-1.83 (m, 2H), 1.79-1.53 (m, 2H), 1.20 (br d, J=6.6 Hz, 3H).

### 2-[3-[[1-(2,6-dioxo-3-piperidyl)-3-methyl-2-oxo-benzimidazol-5-yl]amino]pyrazol-1-yl]-N-[5-fluoro-7-hydroxy-6-(1,1,4-trioxo-1,2,5-thiadiazolidin-2-yl)-2-naphthyl]acetamide (Degrader 107)

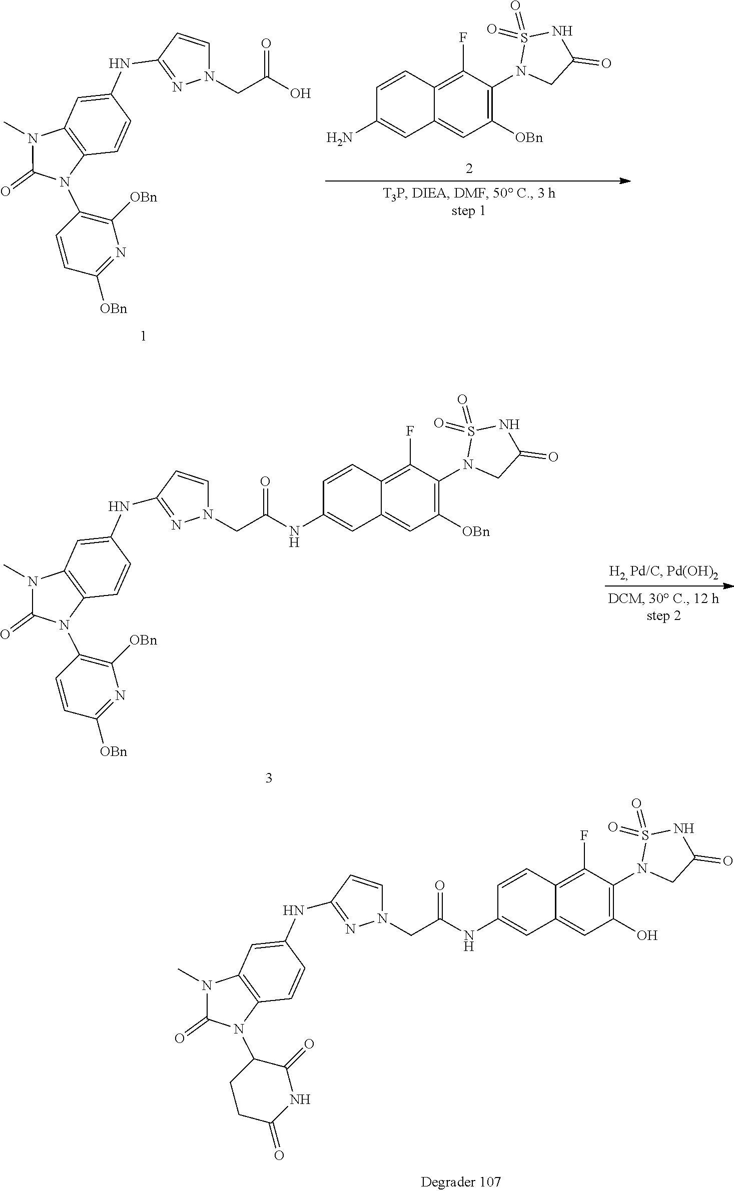 [SMILES: Cn1c(=O)n(-c2ccc(OCc3ccccc3)nc2OCc2ccccc2)c2ccc(Nc3ccn(CC(=O)O)n3)cc21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(Nc3ccn(CC(=O)Nc4ccc5c(F)c(N6CC(=O)NS6(=O)=O)c(OCc6ccccc6)cc5c4)n3)cc21 ; Cn1c(=O)n(C2CCC(=O)NC2=O)c2ccc(Nc3ccn(CC(=O)Nc4ccc5c(F)c(N6CC(=O)NS6(=O)=O)c(O)cc5c4)n3)cc21]

### Step 1: N-[7-benzyloxy-5-fluoro-6-(1,1,4-trioxo-1,2,5-thiadiazolidin-2-yl)-2-naphthyl]-2-[3-[[1-(2,6-dibenzyloxy-3-pyridyl)-3-methyl-2-oxo-benzimidazol-5-yl]amino]pyrazol-1-yl]acetamide (3)

To a solution of 2-[3-[[1-(2,6-dibenzyloxy-3-pyridyl)-3-methyl-2-oxo-benzimidazol-5-yl]amino]pyrazol-1-yl]acetic acid (1, 150 mg, 260.15 μmol) and 5-(6-amino-3-benzyloxy-1-fluoro-2-naphthyl)-1,1-dioxo-1,2,5-thiadiazolidin-3-one (2, 147.50 mg, 286.16 μmol, 061) in DMF (1.5 mL) was added N-ethyl-N-isopropyl-propan-2-amine (336.21 mg, 2.60 mmol, 453.12 μL) and 2,4,6-tripropyl-1,3,5,2$1\hat{ }{5},4$1\hat{ }{5},6$1\hat{ }{5}-trioxatriphosphinane 2,4,6-trioxide (413.87 mg, 1.30 mmol), then the mixture was stirred at 50° C. for 3 hrs. The mixture was concentrated to give N-[7-benzyloxy-5-fluoro-6-(1,1,4-trioxo-1,2,5-thiadiazolidin-2-yl)-2-naphthyl]-2-[3-[[1-(2,6-dibenzyloxy-3-pyridyl)-3-methyl-2-oxo-benzimidazol-5-yl]amino]pyrazol-1-yl]acetamide (3, 70 mg, 70.73 μmol, 27% yield) as a white solid.

LCMS (ES+): m/z 960.4 [M+H]+

### Step 2: 2-[3-[[1-(2,6-dioxo-3-piperidyl)-3-methyl-2-oxo-benzimidazol-5-yl]amino]pyrazol-1-yl]-N-[5-fluoro-7-hydroxy-6-(1,1,4-trioxo-1,2,5-thiadiazolidin-2-yl)-2-naphthyl]acetamide (Degrader 107)

N-[7-benzyloxy-5-fluoro-6-(1,1,4-trioxo-1,2,5-thiadiazolidin-2-yl)-2-naphthyl]-2-[3-[[1-(2,6-dibenzyloxy-3-pyridyl)-3-methyl-2-oxo-benzimidazol-5-yl]amino]pyrazol-1-yl]acetamide (3, 70 mg, 72.92 μmol) was dissolved in DMF (1 mL) then put under nitrogen atmosphere using a cycle of vacuums and nitrogen purges. Pd/C (70 mg, 10% purity) and Pd(OH)₂/C (70 mg, 10% purity) were added to the solution, and the reaction mixture was submitted to a 1 atm hydrogen atmosphere using a cycle of vacuums and hydrogen purges from a rubber balloon. The reaction mixture was stirred at 25° C. for 16 h under H₂ (15 psi). The reaction mixture was filtered, and the filtrate was concentrated under reduced pressure. The residue was purified by prep-HPLC (flow: 25 mL/min; gradient: from 18-48% water (0.1% TFA) in MeCN over 10 min; column: Phenomenex Synergi C18 150×25 mm×10 μm) and lyophilized to give 2-[3-[[1-(2,6-dioxo-3-piperidyl)-3-methyl-2-oxo-benzimidazol-5-yl]amino]pyrazol-1-yl]-N-[5-fluoro-7-hydroxy-6-(1,1,4-trioxo-1,2,5-thiadiazolidin-2-yl)-2-naphthyl]acetamide (Degrader 107, 17.33 mg, 21.29 μmol, 29% yield, TFA salt) as a gray solid.

LC-MS (ESI): m/z 692.3 [M+H]+

¹H NMR (400 MHz, DMSO-d6) δ=11.05 (s, 1H), 10.55 (s, 2H), 8.49-8.26 (m, 1H), 8.19 (s, 1H), 7.90 (d, J=9.0 Hz, 1H), 7.63 (d, J=2.3 Hz, 1H), 7.46 (dd, J=2.0, 9.0 Hz, 1H), 7.22 (d, J=1.7 Hz, 1H), 7.02-6.86 (m, 3H), 5.87 (d, J=2.3 Hz, 1H), 5.27 (br dd, J=5.4, 12.7 Hz, 1H), 4.93 (s, 2H), 4.39 (s, 2H), 3.23 (s, 3H), 2.98-2.87 (m, 1H), 2.76-2.66 (m, 2H), 2.05-1.91 (m, 1H)

### N-(6-(1,1-dioxido-4-oxo-1,2,5-thiadiazolidin-2-yl)-5-fluoro-7-hydroxynaphthalen-2-yl)-2-(((4-(1-(2,6-dioxopiperidin-3-yl)-3-methyl-2-oxo-2,3-dihydro-1H-benzo[d]imidazol-5-yl)phenyl)amino)methyl)cyclopropanecarboxamide (Degrader 108)

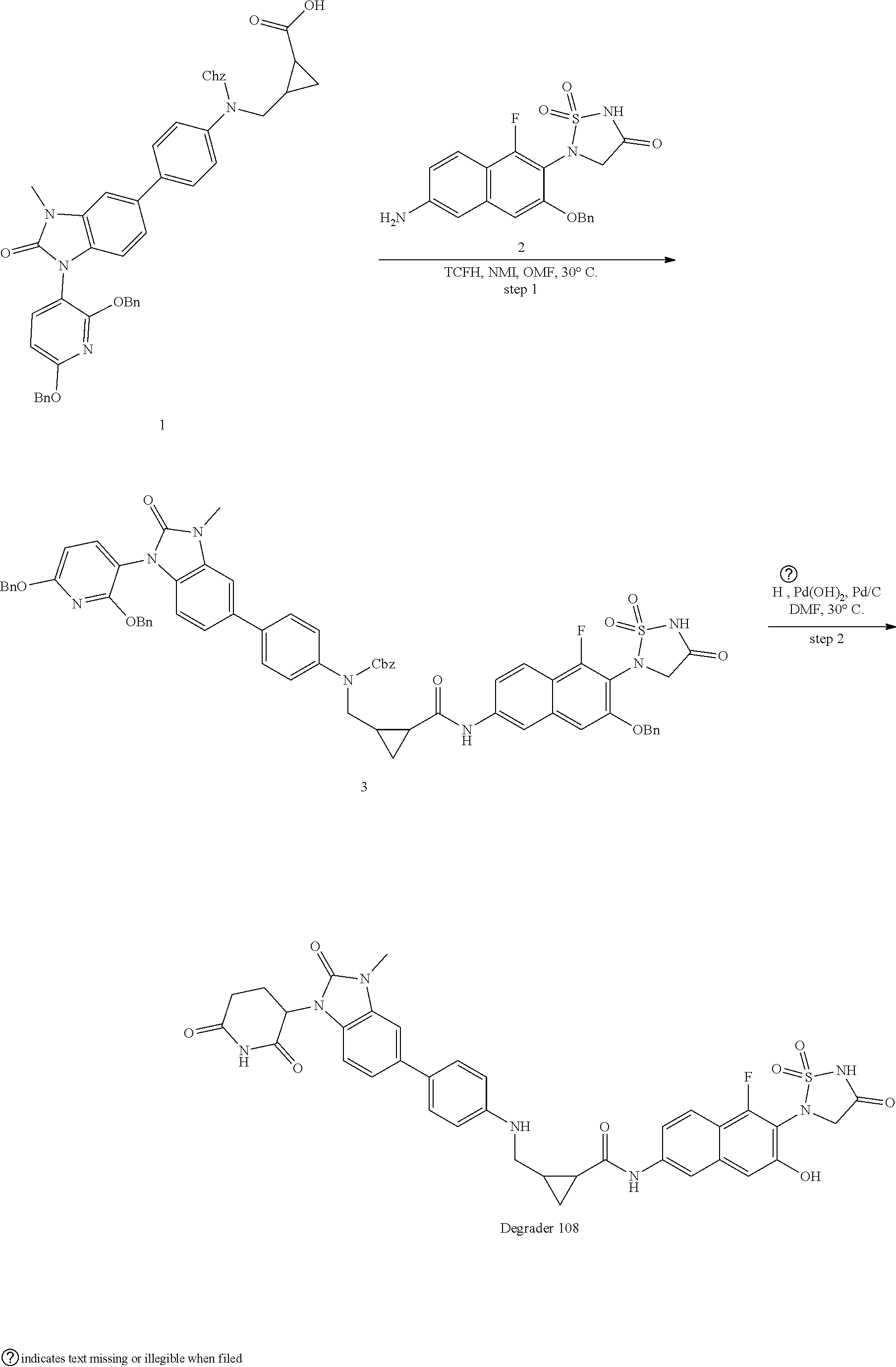

### Step 1: benzyl ((2-((7-(benzyloxy)-6-(1,1-dioxido-4-oxo-1,2,5-thiadiazolidin-2-yl)-5-fluoronaphthalen-2-yl)carbamoyl)cyclopropyl)methyl)(4-(1-(2,6-bis(benzyloxy)pyridin-3-yl)-3-methyl-2-oxo-2,3-dihydro-1H-benzo[d]imidazol-5-yl)phenyl)carbamate (3)

To a mixture of 2-[[N-benzyloxycarbonyl-4-[1-(2,6-dibenzyloxy-3-pyridyl)-3-methyl-2-oxo-benzimidazol-5-yl]anilino]methyl]cyclopropanecarboxylic acid (17, 150 mg, 197.15 μmol), 5-(6-amino-3-(benzyloxy)-1-fluoronaphthalen-2-yl)-1,2,5-thiadiazolidin-3-one 1,1-dioxide (2, 132.11 mg, 256.30 μmol, TFA salt) and NMI (80.93 mg, 985.76 μmol) in DMF (1.5 mL) was added TCFH (138.29 mg, 492.88 μmol) at 0° C. The mixture was stirred at 20° C. for 16 h. The mixture was purified by Prep-HPLC (NH₃·H₂O) and then lyophilization. The benzyl ((2-((7-(benzyloxy)-6-(1,1-dioxido-4-oxo-1,2,5-thiadiazolidin-2-yl)-5-fluoronaphthalen-2-yl)carbamoyl)cyclopropyl)methyl)(4-(1-(2,6-bis(benzyloxy)pyridin-3-yl)-3-methyl-2-oxo-2,3-dihydro-1H-benzo[d]imidazol-5-yl)phenyl)carbamate (3, 100 mg, 86.52 μmol, 44% yield) as white solid.

LCMS (ESI): m/z 1144.2 [M+H]⁺

### Step 2: N-(6-(1,1-dioxido-4-oxo-1,2,5-thiadiazolidin-2-yl)-5-fluoro-7-hydroxynaphthalen-2-yl)-2-(((4-(1-(2,6-dioxopiperidin-3-yl)-3-methyl-2-oxo-2,3-dihydro-1H-benzo[d]imidazol-5-yl)phenyl)amino)methyl)cyclopropanecarboxamide (Degrader 108)

To a solution of benzyl ((2-((7-(benzyloxy)-6-(1,1-dioxido-4-oxo-1,2,5-thiadiazolidin-2-yl)-5-fluoronaphthalen-2-yl)carbamoyl)cyclopropyl)methyl)(4-(1-(2,6-bis(benzyloxy)pyridin-3-yl)-3-methyl-2-oxo-2,3-dihydro-1H-benzo[d]imidazol-5-yl)phenyl)carbamate (3, 100 mg, 87.40 μmol) in DMF (2.5 mL) was added Pd/C (100 mg, 10% purity) and Pd(OH)₂/C (100 mg, 87.40 μmol, 10% purity) under N₂ atmosphere. The suspension was degassed and purged with H₂ 3 times. The mixture was stirred at 30° C. for 16 hr under H₂ (15 psi). The reaction mixture was filtered and concentrated under vacuum. The residue was purified by prep-HPLC (flow: 25 mL/min; gradient: from 8-38% MeCN in water (0.1% TFA) over 12 min; column: Phenomenex SynergiC18 150×25 mm×10 um) to afford N-(6-(1,1-dioxido-4-oxo-1,2,5-thiadiazolidin-2-yl)-5-fluoro-7-hydroxynaphthalen-2-yl)-2-(((4-(1-(2,6-dioxopiperidin-3-yl)-3-methyl-2-oxo-2,3-dihydro-1H-benzo[d]imidazol-5-yl)phenyl)amino)methyl)cyclopropanecarboxamide (Degrader 108, 38.28 mg, 44.73 μmol, 51% yield, TFA salt) as a white solid.

LCMS (ESI): m/z 742.1 [M+H]⁺

¹H NMR (400 MHz, DMSO-d₆) δ11.11 (s, 1H), 10.63-10.27 (m, 2H), 8.21-8.13 (m, 1H), 7.89-7.80 (m, 1H), 7.59-7.42 (m, 3H), 7.41-7.30 (m, 1H), 7.28-7.05 (m, 2H), 7.03-6.73 (m, 3H), 5.45-5.31 (m, 1H), 4.40 (br d, J=2.6 Hz, 2H), 3.50-3.43 (m, 1H), 3.38 (d, J=10.4 Hz, 3H), 3.27-3.03 (m, 1H), 2.99-2.84 (m, 1H), 2.78-2.67 (m, 2H), 2.13-1.95 (m, 2H), 1.90-1.55 (m, 2H), 1.21-0.90 (m, 2H).

### N-(6-(1,1-dioxido-4-oxo-1,2,5-thiadiazolidin-2-yl)-5-fluoro-7-hydroxynaphthalen-2-yl)-2-((1R,3r,5S)-8-(1-(2,6-dioxopiperidin-3-yl)-3-methyl-2-oxo-2,3-dihydro-1H-benzo[d]imidazol-5-yl)-8-azabicyclo[3.2.1]octan-3-yl)acetamide (Degrader 109)

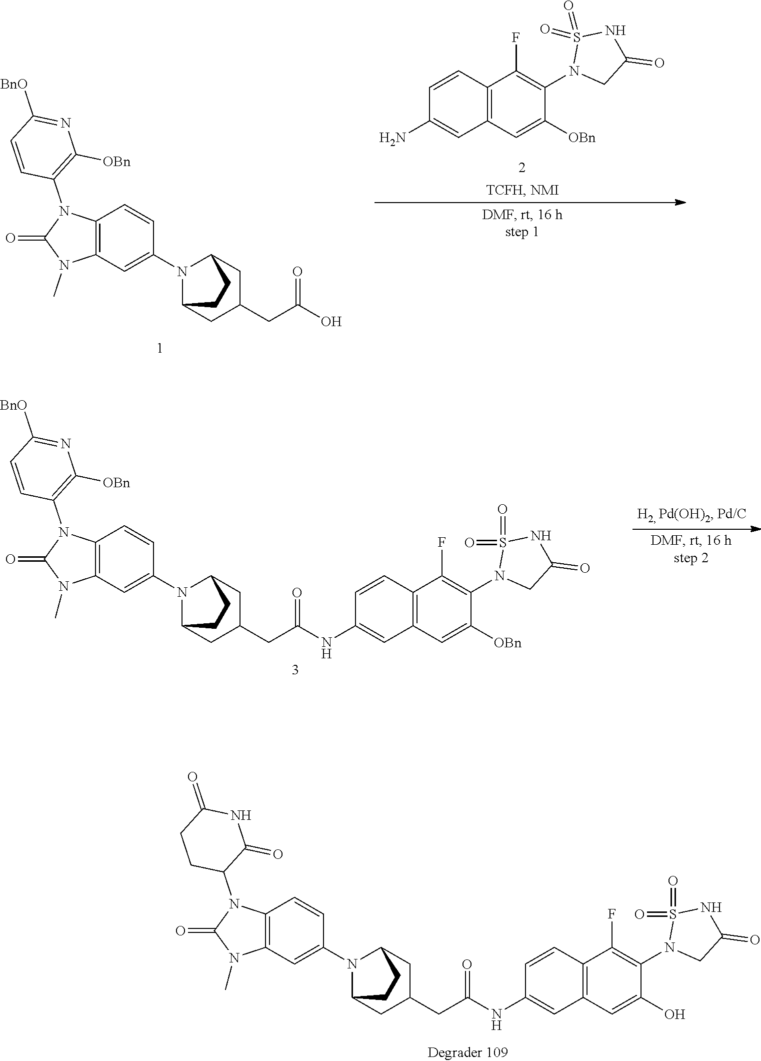

Note: Configurations are arbitrarily assigned.

### Step 1: N-(7-(benzyloxy)-6-(1,1-dioxido-4-oxo-1,2,5-thiadiazolidin-2-yl)-5-fluoronaphthalen-2-yl)-2-((1R,3r,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetamide (3)

To a solution of 2-((1R,3r,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetic acid (1, 80 mg, 132.30 μmol) and 5-(6-amino-3-(benzyloxy)-1-fluoronaphthalen-2-yl)-1,2,5-thiadiazolidin-3-one 1,1-dioxide (2, 58.42 mg, 145.53 μmol, TFA salt) in DMF (2 mL) were added 1-methyl-1H-imidazole (54.31 mg, 661.49 μmol, 52.73 μL) and N-(chloro(dimethylamino)methylene)-N-methylmethanaminium hexafluorophosphate (92.80 mg, 330.75 μmol) at 0° C. The mixture was stirred at 25° C. for 15 h. The reaction mixture was filtered and the filtrate was concentrated under reduced pressure to give residue. The residue was purified by prep-HPLC (flow: 25 mL/min; gradient: from 28-58% MeCN-water (0.05% ammonia hydroxide v/v) over 10 min; column: Waters Xbridge 150×25 mm×5 um) to afford N-(7-(benzyloxy)-6-(1,1-dioxido-4-oxo-1,2,5-thiadiazolidin-2-yl)-5-fluoronaphthalen-2-yl)-2-((1R,3r,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetamide (3, 60 mg, 60.72 μmol, 46% yield) as a white solid.

LCMS (ESI): m/z 988.5 [M+H]⁺

### Step 2: N-(6-(1,1-dioxido-4-oxo-1,2,5-thiadiazolidin-2-yl)-5-fluoro-7-hydroxynaphthalen-2-yl)-2-((1R,3r,5S)-8-(1-(2,6-dioxopiperidin-3-yl)-3-methyl-2-oxo-2,3-dihydro-1H-benzo[d]imidazol-5-yl)-8-azabicyclo[3.2.1]octan-3-yl)acetamide (Degrader 109)

To a solution of N-(7-(benzyloxy)-6-(1,1-dioxido-4-oxo-1,2,5-thiadiazolidin-2-yl)-5-fluoronaphthalen-2-yl)-2-((1R,3r,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetamide (3, 60.00 mg, 60.72 μmol) in DMF (3 mL) were added Pd/C (60 mg, 10% purity) and Pd(OH)₂/C (60 mg, 10% purity) under N₂ atmosphere. The suspension was degassed and purged with H₂ 3 times. The mixture was stirred under H₂ (15 Psi) at 30° C. for 15 h. The reaction mixture was filtered and the filtrate was concentrated under reduced pressure to give residue. The residue was purified by prep-HPLC (flow: 25 mL/min; gradient: from 12-42% MeCN-water (0.1% TFA) over 7 min; column: 3_Phenomenex Luna C18 75×30 mm×3 um) to afford N-(6-(1,1-dioxido-4-oxo-1,2,5-thiadiazolidin-2-yl)-5-fluoro-7-hydroxynaphthalen-2-yl)-2-((1R,3r,5S)-8-(1-(2,6-dioxopiperidin-3-yl)-3-methyl-2-oxo-2,3-dihydro-1H-benzo[d]imidazol-5-yl)-8-azabicyclo[3.2.1]octan-3-yl)acetamide (Degrader 109, 35.42 mg, 39.85 μmol, 66% yield, TFA salt) as a pink solid.

LCMS (ESI): m/z 720.1 [M+H]⁺

¹H NMR (400 MHz, DMSO-d₆) δ=11.07 (s, 1H), 10.40-10.23 (m, 1H), 10.18 (s, 1H), 8.19 (s, 1H), 7.85 (d, J=8.9 Hz, 1H), 7.43 (dd, J=1.7, 9.1 Hz, 1H), 6.97 (s, 2H), 6.89-6.46 (m, 2H), 5.38-5.20 (m, 1H), 4.39-4.29 (m, 4H), 3.32 (s, 3H), 2.96-2.81 (m, 1H), 2.77-2.64 (m, 2H), 2.40-2.15 (m, 4H), 2.13-1.89 (m, 6H), 1.46-1.22 (m, 2H).

### N-(6-(1,1-dioxido-4-oxo-1,2,5-thiadiazolidin-2-yl)-5-fluoro-7-hydroxynaphthalen-2-yl)-2-((1R,3s,5S)-8-(1-(2,6-dioxopiperidin-3-yl)-3-methyl-2-oxo-2,3-dihydro-1H-benzo[d]imidazol-5-yl)-8-azabicyclo[3.2.1]octan-3-yl)acetamide (Degrader 110)

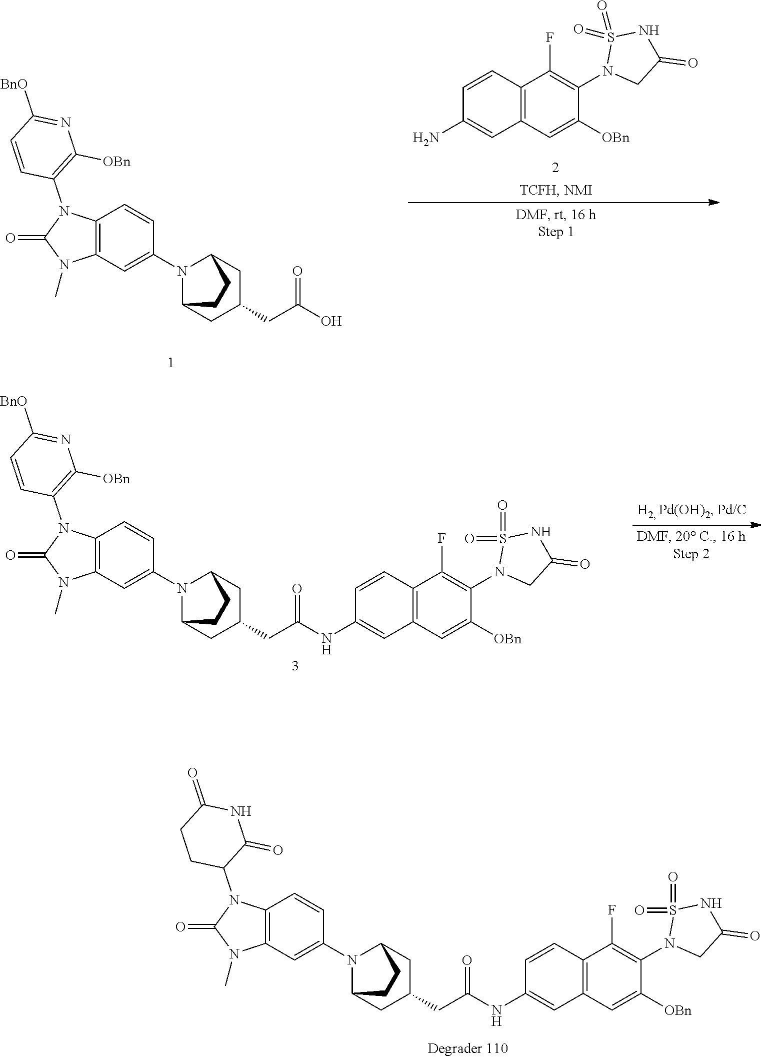

Note: Configurations are arbitrarily assigned.

### Step 1: N-(7-(benzyloxy)-6-(1,1-dioxido-4-oxo-1,2,5-thiadiazolidin-2-yl)-5-fluoronaphthalen-2-yl)-2-((1R,3s,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetamide (3)

To a solution of 2-((1R,3s,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetic acid (1, 150 mg, 248.06 μmol) and 5-(6-amino-3-(benzyloxy)-1-fluoronaphthalen-2-yl)-1,2,5-thiadiazolidin-3-one 1,1-dioxide (2, 109.53 mg, 272.87 μmol, TFA salt) in DMF (2 mL) was added NMI (101.83 mg, 1.24 mmol, 98.86 μL) and TCFH (174.00 mg, 620.15 μmol) at 0° C. The mixture was stirred at 30° C. for 16 h. The reaction mixture was filtered and concentrated under reduced pressure. The reaction solution was purified by prep-HPLC (flow: 25 mL/min; gradient: from 58-28% water (0.05% ammonia hydroxide v/v)-ACN; column: Waters Xbridge 150×25 mm×5 um) to afford N-(7-(benzyloxy)-6-(1,1-dioxido-4-oxo-1,2,5-thiadiazolidin-2-yl)-5-fluoronaphthalen-2-yl)-2-((1R,3s,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetamide (3, 80 mg, 78.45 μmol, 32% yield) as a white solid.

LCMS (ESI): m/z 989.8 [M+H]⁺

### Step 2: N-(6-(1,1-dioxido-4-oxo-1,2,5-thiadiazolidin-2-yl)-5-fluoro-7-hydroxynaphthalen-2-yl)-2-((1R,3s,5S)-8-(1-(2,6-dioxopiperidin-3-yl)-3-methyl-2-oxo-2,3-dihydro-1H-benzo[d]imidazol-5-yl)-8-azabicyclo[3.2.1]octan-3-yl)acetamide (Degrader 110)

To a solution of N-(7-(benzyloxy)-6-(1,1-dioxido-4-oxo-1,2,5-thiadiazolidin-2-yl)-5-fluoronaphthalen-2-yl)-2-((1R,3s,5S)-8-(1-(2,6-bis(benzyloxy)pyridin-3-yl)-3-methyl-2-oxo-2,3-dihydro-1H-benzo[d]imidazol-5-yl)-8-azabicyclo[3.2.1]octan-3-yl)acetamide (3, 80.00 mg, 80.96 μmol) in DMF (3 mL) was added Pd/C (80 mg, 10% purity) and Pd(OH)₂/C (80 mg, 10% purity). The mixture was stirred at 30° C. for 15 h under H₂ (15 Psi). The reaction mixture was filtered and concentrated under reduced pressure to give a residue. The residue was purified by prep-HPLC (flow: 25 mL/min; gradient: from 42%-12% water (0.1% TFA)-ACN; column: 3_Phenomenex Luna C18 75×30 mm×3 um) to afford N-(6-(1,1-dioxido-4-oxo-1,2,5-thiadiazolidin-2-yl)-5-fluoro-7-hydroxynaphthalen-2-yl)-2-((1R,3s,5S)-8-(1-(2,6-dioxopiperidin-3-yl)-3-methyl-2-oxo-2,3-dihydro-1H-benzo[d]imidazol-5-yl)-8-azabicyclo[3.2.1]octan-3-yl)acetamide (Degrader 110, 36.49 mg, 42.01 μmol, 52% yield, TFA salt) as a pink solid.

LCMS (ESI): m/z 720.4 [M+H]⁺

¹H NMR (400 MHz, DMSO-d₆) δ=11.08 (s, 1H), 10.47-10.20 (m, 1H), 10.11-9.92 (m, 1H), 8.16 (s, 1H), 7.81 (d, J=8.9 Hz, 1H), 7.36 (br d, J=8.8 Hz, 1H), 7.09-6.35 (m, 4H), 5.36-5.21 (m, 1H), 4.35 (s, 2H), 4.41-4.30 (m, 1H), 3.30 (s, 3H), 2.98-2.80 (m, 1H), 2.75-2.57 (m, 2H), 2.18-1.94 (m, 6H), 1.83 (br d, J=6.7 Hz, 3H), 1.69-1.32 (m, 4H).

### N-(6-(1,1-dioxido-4-oxo-1,2,5-thiadiazolidin-2-yl)-5-fluoro-7-hydroxynaphthalen-2-yl)-2-((3S,4S)-4-(1-(2,6-dioxopiperidin-3-yl)-3-ethyl-2-oxo-2,3-dihydro-1H-benzo[d]imidazol-5-yl)-3-hydroxypiperidin-1-yl)acetamide (Degrader 111)

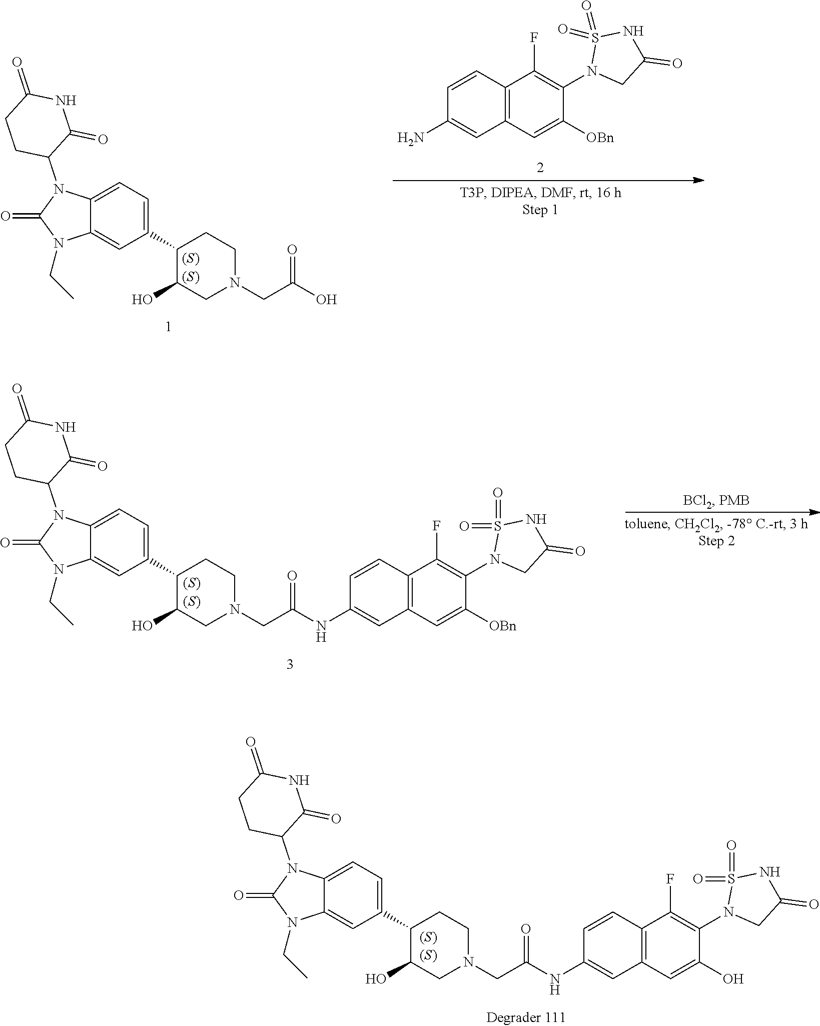

Note: Configurations are arbitrarily assigned.

### Step 1: N-(7-(benzyloxy)-6-(1,1-dioxido-4-oxo-1,2,5-thiadiazolidin-2-yl)-5-fluoronaphthalen-2-yl)-2-((3S,4S)-4-(1-(2,6-dioxopiperidin-3-yl)-3-ethyl-2-oxo-2,3-dihydro-1H-benzo[d]imidazol-5-yl)-3-hydroxypiperidin-1-yl)acetamide (3)

Into a 50 mL single-neck round-bottom flask containing a well-stirred solution of 2-[(3S,4S)-4-[1-(2,6-dioxo-3-piperidyl)-3-ethyl-2-oxo-benzimidazol-5-yl]-3-hydroxy-1-piperidyl]acetic acid (1, 180 mg, 343.10 μmol, HCl salt) and 5-(6-amino-3-benzyloxy-1-fluoro-2-naphthyl)-1,1-dioxo-1,2,5-thiadiazolidin-3-one (2, 164.09 mg, 308.79 μmol, TFA salt) in anhydrous DMF (3 mL) were added DIPEA (443.43 mg, 3.43 mmol, 597.61 μL) and propylphosphonic anhydride solution (50 Wt % in EtOAc, 436.67 mg, 686.21 μmol, 50% purity). The resultant reaction mixture was stirred at room temperature for 16 h. The solvent was evaporated and the resulting crude was purified by reverse-phase column chromatography [Redisep C18-120 g; Mobile Phase A: 0.1% FA in Water and Mobile Phase B: CH₃CN] to afford N-[7-benzyloxy-5-fluoro-6-(1,1,4-trioxo-1,2,5-thiadiazolidin-2-yl)-2-naphthyl]-2-[(3S,4S)-4-[1-(2,6-dioxo-3-piperidyl)-3-ethyl-2-oxo-benzimidazol-5-yl]-3-hydroxy-1-piperidyl]acetamide (3, 95 mg, 102.75 μmol, 30% yield, formic acid salt) as an off-white solid.

LCMS (ES+): m/z 814.0 [M+H]⁺

### Step 2: N-(6-(1,1-dioxido-4-oxo-1,2,5-thiadiazolidin-2-yl)-5-fluoro-7-hydroxynaphthalen-2-yl)-2-((3S,4S)-4-(1-(2,6-dioxopiperidin-3-yl)-3-ethyl-2-oxo-2,3-dihydro-1H-benzo[d]imidazol-5-yl)-3-hydroxypiperidin-1-yl)acetamide (Degrader 111)

Into a 100 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[(3S,4S)-4-[1-(2,6-dioxo-3-piperidyl)-3-ethyl-2-oxo-benzimidazol-5-yl]-3-hydroxy-1-piperidyl]acetamide (3, 95 mg, 102.75 μmol, formic acid salt) and pentamethylbenzene (76.16 mg, 513.74 μmol) in anhydrous toluene (10 mL) and DCM (10 mL) was added BCl₃ (1.0 M solution in DCM, 2.57 mmol, 2.6 mL) at −78° C. The resultant reaction mixture was stirred at ambient temperature for 3 h. The reaction mixture was quenched with 5 mL of 5% MeOH in DCM at −78° C. and concentrated under reduced pressure to get the crude material that was purified by reverse-phase preparative-HPLC [Column: X-BRIDGE C8 (150×19) mm, 5 μm; Mobile phase A: 0.1% FA in water and Mobile Phase B: CH₃CN] to afford N-(6-(1,1-dioxido-4-oxo-1,2,5-thiadiazolidin-2-yl)-5-fluoro-7-hydroxynaphthalen-2-yl)-2-((3S,4S)-4-(1-(2,6-dioxopiperidin-3-yl)-3-ethyl-2-oxo-2,3-dihydro-1H-benzo[d]imidazol-5-yl)-3-hydroxypiperidin-1-yl)acetamide (Degrader 111, 23 mg, 29.52 μmol, 29% yield, formic acid salt) as a white solid.

¹H-NMR (400 MHz, DMSO-d6): δ 11.15 (s, 1H), 10.82 (brs, 1H), 10.06 (brs, 1H), 9.86 (s, 1H), 8.12 (s, 1H), 7.91 (d, J=9.20 Hz, 1H), 7.49 (d, J=9.20 Hz, 1H), 7.10-7.08 (m, 2H), 7.01 (s, 1H), 6.94 (d, J=8.00 Hz, 1H), 5.40-5.35 (m, 2H), 4.40-4.25 (m, 2H), 4.19-4.00 (m, 3H), 3.91-3.88 (m, 2H), 3.70-3.55 (m, 1H), 3.30-3.15 (m, 1H), 3.10-2.85 (m, 2H), 2.73-2.56 (m, 3H), 2.20-1.90 (m, 3H), 1.26 (t, J=7.20 Hz, 3H).

LCMS (ES−): m/z 722.2 [M−H]⁻

### N-(6-(1,1-dioxido-4-oxo-1,2,5-thiadiazolidin-2-yl)-5-fluoro-7-hydroxynaphthalen-2-yl)-2-((3R,4R)-4-(1-(2,6-dioxopiperidin-3-yl)-3-ethyl-2-oxo-2,3-dihydro-1H-benzo[d]imidazol-5-yl)-3-hydroxypiperidin-1-yl)acetamide (Degrader 112)

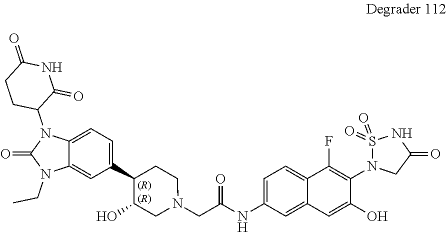

### Step 1: N-(7-(benzyloxy)-6-(1,1-dioxido-4-oxo-1,2,5-thiadiazolidin-2-yl)-5-fluoronaphthalen-2-yl)-2-((3R,4R)-4-(1-(2,6-dioxopiperidin-3-yl)-3-ethyl-2-oxo-2,3-dihydro-1H-benzo[d]imidazol-5-yl)-3-hydroxypiperidin-1-yl)acetamide (3)

Into a 50 mL single-neck round-bottom flask containing a well-stirred solution of 2-[(3R,4R)-4-[1-(2,6-dioxo-3-piperidyl)-3-ethyl-2-oxo-benzimidazol-5-yl]-3-hydroxy-1-piperidyl]acetic acid (1, 170 mg, 353.17 μmol, HCl salt) and 5-(6-amino-3-benzyloxy-1-fluoro-2-naphthyl)-1,1-dioxo-1,2,5-thiadiazolidin-3-one (2, 168.90 mg, 317.85 μmol, TFA salt) in anhydrous DMF (3 mL) were added DIPEA (456.44 mg, 3.53 mmol, 615.14 μL) and propylphosphonic anhydride solution (50 Wt % in EtOAc, 449.49 mg, 706.34 μmol, 50% purity). The resultant reaction mixture was stirred at room temperature for 16 h. The solvent was evaporated and the resulting crude was purified by reverse-phase column chromatography [Redisep C18-120 g; Mobile Phase A: 0.1% FA in Water and Mobile Phase B: CH₃CN] to afford N-[7-benzyloxy-5-fluoro-6-(1,1,4-trioxo-1,2,5-thiadiazolidin-2-yl)-2-naphthyl]-2-[(3R,4R)-4-[1-(2,6-dioxo-3-piperidyl)-3-ethyl-2-oxo-benzimidazol-5-yl]-3-hydroxy-1-piperidyl]acetamide (3, 130 mg, 139.85 μmol, 40% yield, formic acid salt) as an off-white solid.

LCMS (ES+): m/z 814.9 [M+H]⁺

### Step 2: N-(6-(1,1-dioxido-4-oxo-1,2,5-thiadiazolidin-2-yl)-5-fluoro-7-hydroxynaphthalen-2-yl)-2-((3R,4R)-4-(1-(2,6-dioxopiperidin-3-yl)-3-ethyl-2-oxo-2,3-dihydro-1H-benzo[d]imidazol-5-yl)-3-hydroxypiperidin-1-yl)acetamide (Degrader 112)

Into a 100 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[(3R,4R)-4-[1-(2,6-dioxo-3-piperidyl)-3-ethyl-2-oxo-benzimidazol-5-yl]-3-hydroxy-1-piperidyl]acetamide (3, 150 mg, 161.36 μmol, formic acid salt) and pentamethylbenzene (119.60 mg, 806.80 μmol) in anhydrous toluene (10 mL) and DCM (10 mL) was added BCl₃ (1.0 M solution in DCM, 4.03 mmol, 4.0 mL) at −78° C. The resultant reaction mixture was stirred at ambient temperature for 3 h. The reaction mixture was quenched with 5 mL of 5% MeOH in DCM at −78° C. and concentrated under reduced pressure to get the crude material that was purified by reverse-phase preparative-HPLC [Column: X-BRIDGE C8 (150×19) mm, 5 μm; Mobile phase A: 0.1% FA in water and Mobile Phase B: CH₃CN] to afford N-(6-(1,1-dioxido-4-oxo-1,2,5-thiadiazolidin-2-yl)-5-fluoro-7-hydroxynaphthalen-2-yl)-2-((3R,4R)-4-(1-(2,6-dioxopiperidin-3-yl)-3-ethyl-2-oxo-2,3-dihydro-1H-benzo[d]imidazol-5-yl)-3-hydroxypiperidin-1-yl)acetamide (Degrader 112, 40 mg, 51.05 μmol, 32% yield, formic acid salt) as a white solid.

¹H-NMR (400 MHz, DMSO-d6): δ 11.12 (s, 1H), 10.81 (brs, 1H), 10.02 (brs, 1H), 9.87 (s, 1H), 8.12 (s, 1H), 7.92 (d, J=8.80 Hz, 1H), 7.48 (d, J=8.80 Hz, 1H), 7.10-7.07 (m, 2H), 7.01 (s, 1H), 6.94 (d, J=8.00 Hz, 1H), 5.40-5.35 (m, 2H), 4.40-4.25 (m, 2H), 4.15-4.00 (m, 3H), 3.91-3.88 (m, 2H), 3.70-3.55 (m, 1H), 3.30-3.15 (m, 1H), 3.10-2.85 (m, 2H), 2.73-2.56 (m, 3H), 2.20-1.90 (m, 3H), 1.26 (t, J=7.20 Hz, 3H),

LCMS (ES−): m/z 722.0 [M−H]⁻

### N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-3-oxopiperazin-1-yl)acetamide (Degrader 113)

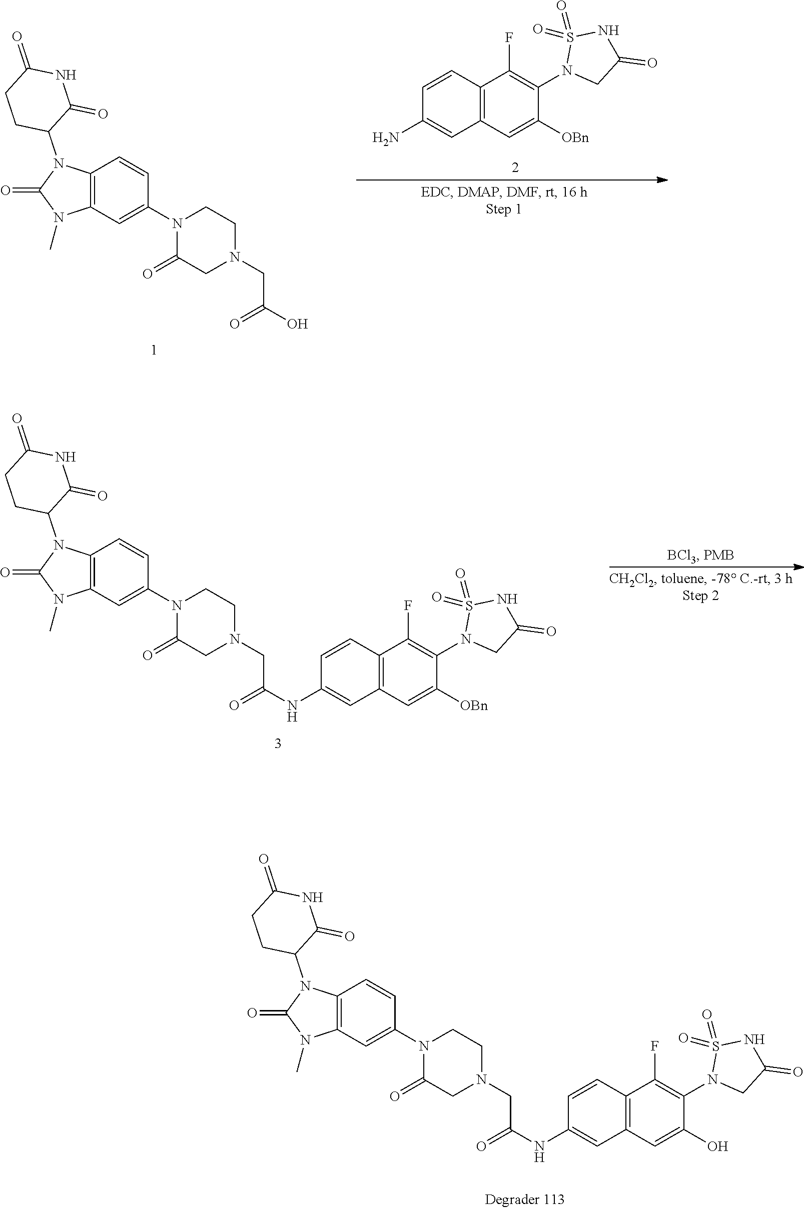 [SMILES: Cn1c(=O)n(C2CCC(=O)NC2=O)c2ccc(N3CCN(CC(=O)O)CC3=O)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(N3CCN(CC(=O)Nc4ccc5c(F)c(N6CC(=O)NS6(=O)=O)c(OCc6ccccc6)cc5c4)CC3=O)cc21 ; Cn1c(=O)n(C2CCC(=O)NC2=O)c2ccc(N3CCN(CC(=O)Nc4ccc5c(F)c(N6CC(=O)NS6(=O)=O)c(O)cc5c4)CC3=O)cc21]

### Step 1: N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)-3-oxopiperazin-1-yl)acetamide (3)

Into a 50 mL single-neck round-bottom flask containing a well-stirred solution of 2-[4-[1-(2,6-dioxo-3-piperidyl)-3-methyl-2-oxo-benzimidazol-5-yl]-3-oxo-piperazin-1-yl]acetic acid (1, 240 mg, 467.40 μmol, HCl salt) in anhydrous DMF (1 mL) were added EDC·HCl (268.80 mg, 1.40 mmol) and DMAP (285.51 mg, 2.34 mmol). The resultant reaction mixture was stirred at ambient temperature for 1 h. Subsequently, 5-(6-amino-3-benzyloxy-1-fluoro-2-naphthyl)-1,1-dioxo-1,2,5-thiadiazolidin-3-one (2, 223.53 mg, 420.66 μmol, TFA salt) was added and stirring was continued for another 16 h at room temperature. Afterwards the solvent was evaporated under reduced pressure and the resulting crude material was purified by reverse-phase column chromatography [Column: Redisep-C18-120 g; Mobile Phase A: 0.1% Formic acid in water and Mobile Phase B: CH₃CN] to afford N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3-oxo-piperazin-1-yl]acetamide (3, 200 mg, 100.85 μmol, 22% yield, formic acid salt) as an off-white solid.

LCMS (ES+): m/z 799.1 [M+H]⁺

### Step 2: N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-3-oxopiperazin-1-yl)acetamide (Degrader 113)

Into a 100 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3-oxo-piperazin-1-yl]acetamide (3, 200 mg, 99.43 μmol, formic acid salt) and pentamethylbenzene (73.70 mg, 497.15 μmol) in anhydrous toluene (6 mL) and DCM (6 mL) was added BCl₃ (1.0 M solution in Methylene chloride, 2.49 mmol, 2.5 mL) at −78° C. The resultant reaction mixture was stirred at room temperature for 16 h. The reaction mixture was quenched with 5 mL of 5% MeOH in DCM at −78° C., concentrated under reduced pressure to get the crude material that was purified by reverse-phase preparative-HPLC [Column: X-BRIDGE C18 (150×10) mm, 5 μm, Mobile phase A: 0.1% Formic acid in water and Mobile Phase B: CH₃CN] to afford N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-3-oxopiperazin-1-yl)acetamide (Degrader 113, 6.0 mg, 7.85 μmol, 8% yield, formic acid salt) as a white solid. ¹H-NMR (400 MHz, DMSO-d6): δ 11.13 (s, 1H), 10.35 (brs, 1H), 9.99 (s, 1H), 8.17 (s, 1H), 7.88 (d, J=8.80 Hz, 1H), 7.55 (dd, J=1.60, 9.20 Hz, 1H), 7.21 (s, 1H), 7.17 (d, J=8.40 Hz, 1H), 7.02-6.96 (m, 2H), 5.40 (dd, J=5.60, 12.80 Hz, 1H), 4.19 (s, 2H), 3.79 (s, 2H), 3.75-3.55 (m, 3H), 2.99-2.75 (m, 1H), 2.82-2.61 (m, 2H), 2.10-2.02 (m, 1H).

[Note: 6 aliphatic protons including a methyl group are eclipsed by the solvent signal]

LCMS (ES+): m/z 709.0 [M+H]⁺

### 3-[5-[4-[2-[(3S)-3-[8-fluoro-6-hydroxy-7-(1,1,4-trioxo-1,2,5-thiadiazolidin-2-yl)-2-naphthyl]pyrrolidin-1-yl]-2-oxo-ethyl]-4-hydroxy-1-piperidyl]-3-methyl-2-oxo-benzimidazol-1-yl]piperidine-2,6-dione (Degrader 114)

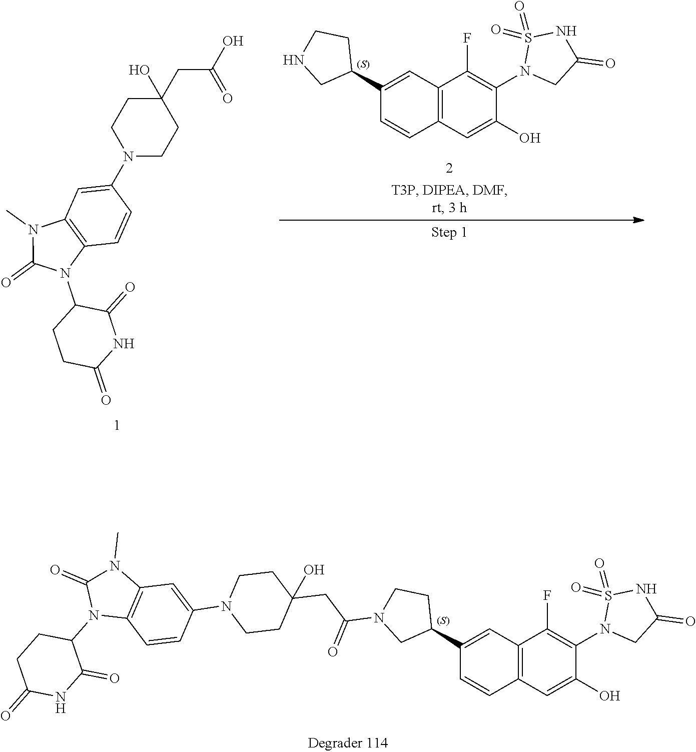

### Step 1: 3-[5-[4-[2-[(3S)-3-[8-fluoro-6-hydroxy-7-(1,1,4-trioxo-1,2,5-thiadiazolidin-2-yl)-2-naphthyl]pyrrolidin-1-yl]-2-oxo-ethyl]-4-hydroxy-1-piperidyl]-3-methyl-2-oxo-benzimidazol-1-yl]piperidine-2,6-dione (Degrader 114)

Note: Configurations are arbitrarily assigned

Into a 50 mL single neck round-bottom flask containing a well-stirred solution of 2-[1-[1-(2,6-dioxo-3-piperidyl)-3-methyl-2-oxo-benzimidazol-5-yl]-4-hydroxy-4-piperidyl]acetic acid (1, 130 mg, 242.62 μmol, TFA salt) in anhydrous DMF (1.5 mL) were added ≥50 wt. % propylphosphonic anhydride solution in ethyl acetate (463.19 mg, 727.87 μmol, 436.97 μL, 50% purity) and DIPEA (156.79 mg, 1.21 mmol, 211.30 μL) at room temperature. The contents were stirred at room temperature for 10 min. Then a solution of 5-[1-fluoro-3-hydroxy-7-[(3S)-pyrrolidin-3-yl]-2-naphthyl]-1,1-dioxo-1,2,5-thiadiazolidin-3-one (2, 104.29 mg, 184.92 μmol, TFA salt) in anhydrous DMF (1.5 mL) was added and stirring continued for 3 h. The solvent was removed under reduced pressure and the residue was suspended in water (30 mL). The precipitate was filtered and purified by reverse phase column chromatography [Silicycle C18 column; Mobile phase A: 0.1% TFA in water; Mobile phase B: MeCN] to get 3-[5-[4-[2-[(3S)-3-[8-fluoro-6-hydroxy-7-(1,1,4-trioxo-1,2,5-thiadiazolidin-2-yl)-2-naphthyl]pyrrolidin-1-yl]-2-oxo-ethyl]-4-hydroxy-1-piperidyl]-3-methyl-2-oxo-benzimidazol-1-yl]piperidine-2,6-dione (Degrader 114, 70 mg, 73.55 μmol, 30% yield, TFA salt) as off white solid.

LCMS (ES+): m/z 764.2 [M+H]⁺

¹HNMR (400 MHz, DMSO-d6): δ 11.13 (s, 1H), 10.00 (brs, 1H), 7.78-7.73 (m, 2H), 7.51-7.47 (m, 1H), 7.23-6.97 (m, 3H), 5.40-5.36 (m, 1H), 4.20 (s, 2H), 4.10 (t, J=8.40 Hz, 2H), 3.95-3.90 (m, 1H), 3.78-3.54 (m, 2H), 3.37-3.31 (m, 7H), 2.94-2.87 (m, 1H), 2.75-2.61 (m, 4H), 2.45-2.38 (m, 1H), 2.17-2.19 (m, 4H), 1.99-1.87 (m, 2H). Additional protons under solvent peaks.

### 3-[5-[4-[2-[(3R)-3-[8-fluoro-6-hydroxy-7-(1,1,4-trioxo-1,2,5-thiadiazolidin-2-yl)-2-naphthyl]pyrrolidin-1-yl]-2-oxo-ethyl]-4-hydroxy-1-piperidyl]-3-methyl-2-oxo-benzimidazol-1-yl]piperidine-2,6-dione (Degrader 115)

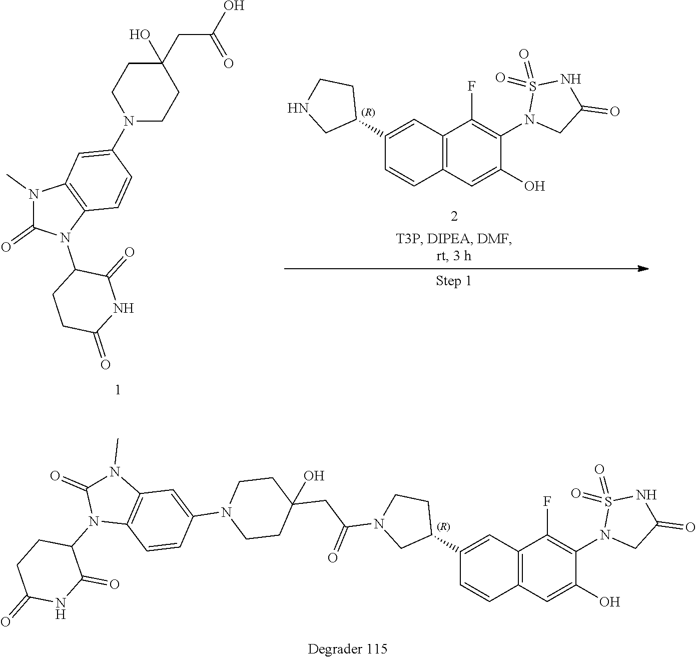

3-[5-[4-[2-[(3R)-3-[8-fluoro-6-hydroxy-7-(1,1,4-trioxo-1,2,5-thiadiazolidin-2-yl)-2-naphthyl]pyrrolidin-1-yl]-2-oxo-ethyl]-4-hydroxy-1-piperidyl]-3-methyl-2-oxo-benzimidazol-1-yl]piperidine-2,6-dione (Degrader 115, 70 mg, 76.31 μmol, TFA salt) was synthesized following the same procedure as 3-[5-[4-[2-[(3S)-3-[8-fluoro-6-hydroxy-7-(1,1,4-trioxo-1,2,5-thiadiazolidin-2-yl)-2-naphthyl]pyrrolidin-1-yl]-2-oxo-ethyl]-4-hydroxy-1-piperidyl]-3-methyl-2-oxo-benzimidazol-1-yl]piperidine-2,6-dione (Degrader 114).

LCMS (ES+): m/z 764.2 [M+H]⁺

¹HNMR (400 MHz, DMSO-d6): δ 11.13 (s, 1H), 10.00 (s, 1H), 7.78-7.73 (m, 2H), 7.51-7.47 (m, 1H), 7.22-6.97 (m, 3H), 5.40-5.36 (m, 1H), 4.30 (s, 2H), 4.21-4.08 (m, 1H), 3.95-3.90 (m, 1H), 3.78-3.54 (m, 2H), 3.54-3.38 (m, 7H), 2.94-2.87 (m, 1H), 2.75-2.58 (m, 4H), 2.45-2.38 (m, 1H), 2.17-2.19 (m, 4H), 1.99-1.81 (m, 2H).

### 3-(5-(1-(2-((S)-3-((7-(1,1-dioxido-4-oxo-1,2,5-thiadiazolidin-2-yl)-8-fluoro-6-hydroxynaphthalen-2-yl)oxy)pyrrolidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (Degrader 116)

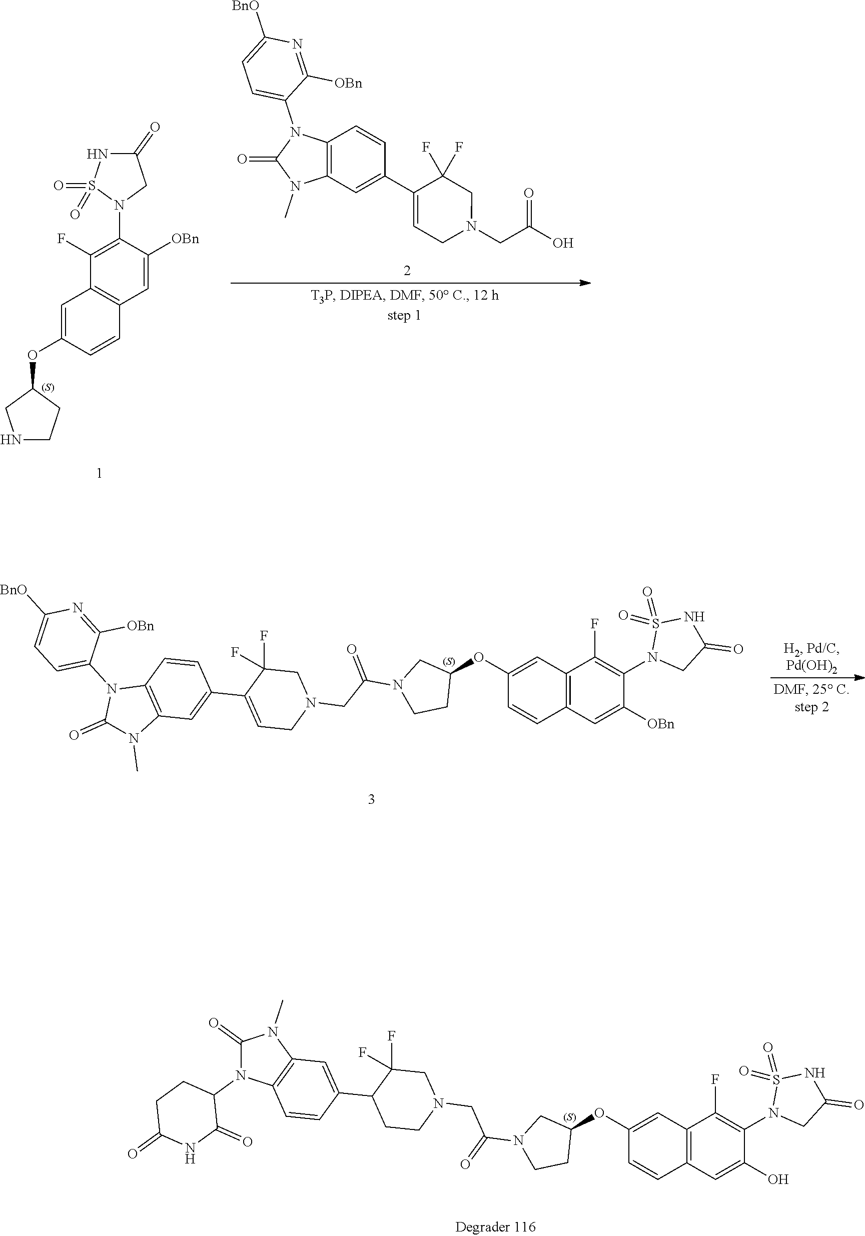

Note: Configurations are arbitrarily assigned.

### Step 1: 3-(5-(1-(2-((S)-3-((6-(benzyloxy)-7-(1,1-dioxido-4-oxo-1,2,5-thiadiazolidin-2-yl)-8-fluoronaphthalen-2-yl)oxy)pyrrolidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (3)

To a solution of (S)-5-(3-(benzyloxy)-1-fluoro-7-(pyrrolidin-3-yloxy)naphthalen-2-yl)-1,2,5-thiadiazolidin-3-one 1,1-dioxide (2, 44 mg, 86.62 μmol, HCl salt) 2-(4-(1-(2,6-dioxopiperidin-3-yl)-3-methyl-2-oxo-2,3-dihydro-1H-benzo[d]imidazol-5-yl)-3,3-difluoropiperidin-1-yl)acetic acid (1, 50 mg, 86.62 μmol) in DMF (1.5 mL) was added N-ethyl-N-isopropylpropan-2-amine (167.93 mg, 1.30 mmol, 226.32 μL) and 2,4,6-tripropyl-1,3,5,2,4,6-trioxatriphosphinane 2,4,6-trioxide (385.86 mg, 606.35 μmol, 360.00 μL, 50% purity). The mixture was stirred at 50° C. for 12 h, it was concentrated in vacuo. The residue was purified by prep-HPLC (flow: 25 mL/min; gradient: from 18-38% water (0.1% NH₄HCO₃) in MeCN over 7 min; column: PhenomenexLunaC1875×30 mm×30 μm) to give (S)-5-(3-(benzyloxy)-7-((1-(2-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)acetyl)pyrrolidin-3-yl)oxy)-1-fluoronaphthalen-2-yl)-1,2,5-thiadiazolidin-3-one 1,1-dioxide (3, 25 mg, 21.57 μmol, 25% yield) LC-MS (ESI): m/z 1066.1 [M+H]⁺

### Step 2: 3-(5-(1-(2-((S)-3-((7-(1,1-dioxido-4-oxo-1,2,5-thiadiazolidin-2-yl)-8-fluoro-6-hydroxynaphthalen-2-yl)oxy)pyrrolidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (Degrader 116)

To a solution of (S)-5-(3-(benzyloxy)-7-((1-(2-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)acetyl)pyrrolidin-3-yl)oxy)-1-fluoronaphthalen-2-yl)-1,2,5-thiadiazolidin-3-one 1,1-dioxide (3, 25 mg, 23.45 μmol) in DMF (3 mL) was added Pd(OH)₂/C (25 mg, 23.45 μmol, 10% purity) and Pd/C (25 mg, 20.58 μmol, 10% purity) under H₂ (15 psi), the mixture was stirred at 30° C. for 16 h under H₂ (15 psi) atmosphere. The mixture was filtered and concentrated in vacuum. The residue was purified by prep-HPLC(Column: 3_Phenomenex Luna C18 75*30 mm*3 um, water (0.1% TFA)-ACN, FlowRate (25 ml/min)) to obtain 3-(5-(1-(2-((S)-3-((7-(1,1-dioxido-4-oxo-1,2,5-thiadiazolidin-2-yl)-8-fluoro-6-hydroxynaphthalen-2-yl)oxy)pyrrolidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (Degrader 116, 3.59 mg, 3.93 μmol, 17% yield, TFA salt) as a gray solid LCMS (ESI): m/z 800.1 [M+H]⁺

¹H NMR (400 MHz, DMSO-d6) δ=11.11 (s, 1H), 9.86-9.57 (m, 1H), 7.72 (br d, J=9.0 Hz, 1H), 7.30-6.87 (m, 6H), 5.47-5.16 (m, 2H), 4.22-4.10 (m, 3H), 3.93-3.85 (m, 1H), 3.82-3.55 (m, 6H), 3.23-3.08 (m, 3H), 3.00-2.82 (m, 2H), 2.78-2.60 (m, 3H), 2.30-2.13 (m, 4H), 2.09-1.81 (m, 2H)

### 3-(5-(1-(2-((R)-3-((7-(1,1-dioxido-4-oxo-1,2,5-thiadiazolidin-2-yl)-8-fluoro-6-hydroxynaphthalen-2-yl)oxy)pyrrolidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (Degrader 117)

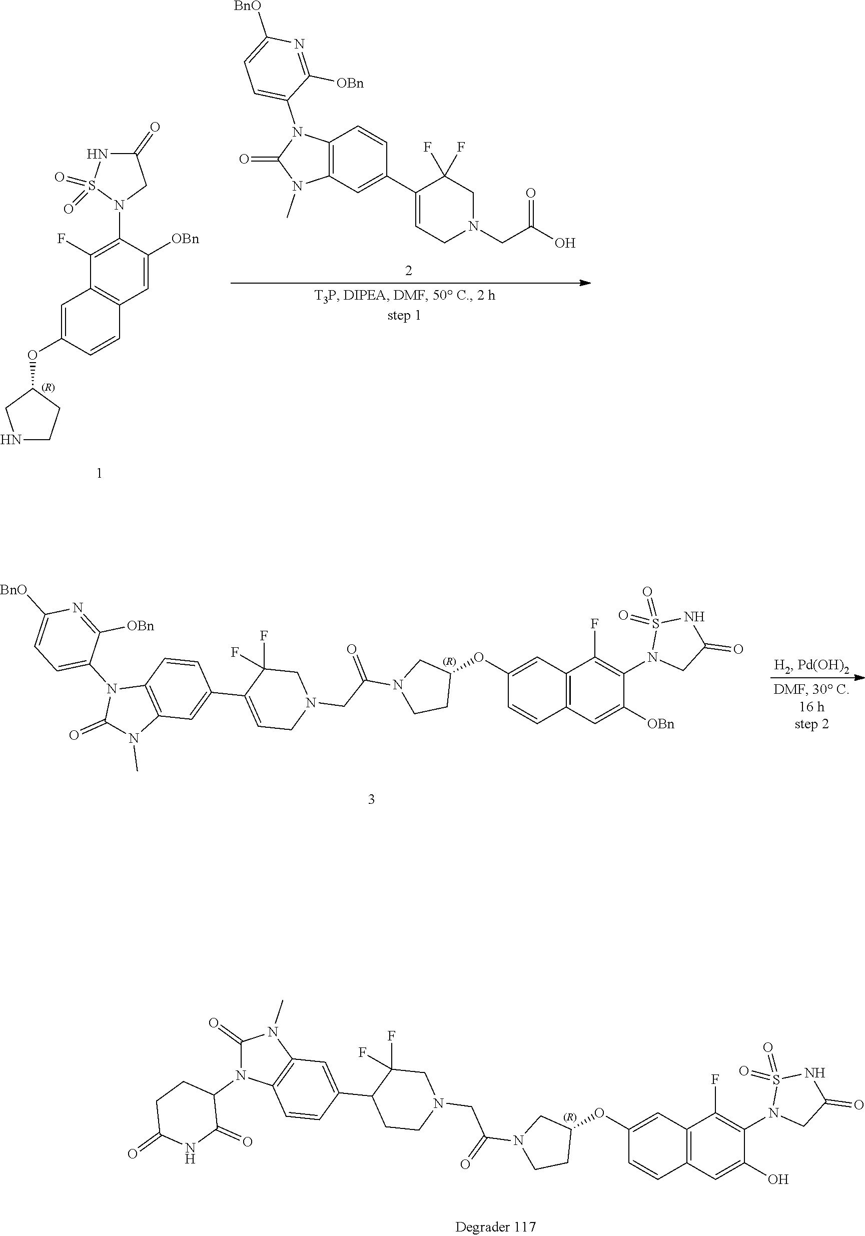

Note: Configurations are arbitrarily assigned.

### Step 1: (R)-5-(3-(benzyloxy)-7-((1-(2-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)acetyl)pyrrolidin-3-yl)oxy)-1-fluoronaphthalen-2-yl)-1,2,5-thiadiazolidin-3-one 1,1-dioxide (3)

To a solution of 5-[3-benzyloxy-1-fluoro-7-[(3R)-pyrrolidin-3-yl]oxy-2-naphthyl]-1,1-dioxo-1,2,5-thiadiazolidin-3-one (1, 150 mg, 318.13 μmol) and 2-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)acetic acid (2, 194.90 mg, 318.13 μmol) in DMF (1.5 mL) were added DIPEA (205.58 mg, 1.59 mmol, 277.07 μL) and T₃P (607.34 mg, 954.40 μmol, 50% purity). The mixture was stirred at 50° C. for 2 h. The reaction was quenched by water (1 mL) to get a mixture. The mixture was purified by reversed phase HPLC (NH₃·H₂O) and concentrated to get (R)-5-(3-(benzyloxy)-7-((1-(2-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)acetyl)pyrrolidin-3-yl)oxy)-1-fluoronaphthalen-2-yl)-1,2,5-thiadiazolidin-3-one 1,1-dioxide (3, 120 mg, 108.06 μmol, 34% yield) as yellow solid.

LCMS (ESI): m/z 1066.1 [M+H]⁺

SFC: ee % 100%

### Step 2: 3-(5-(1-(2-((R)-3-((7-(1,1-dioxido-4-oxo-1,2,5-thiadiazolidin-2-yl)-8-fluoro-6-hydroxynaphthalen-2-yl)oxy)pyrrolidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (Degrader 117)

To a solution of (R)-5-(3-(benzyloxy)-7-((1-(2-(4-(1-(2,6-bis(benzyloxy)pyridin-3-yl)-3-methyl-2-oxo-2,3-dihydro-1H-benzo[d]imidazol-5-yl)-5,5-difluoro-5,6-dihydropyridin-1 (2H)-yl)acetyl)pyrrolidin-3-yl)oxy)-1-fluoronaphthalen-2-yl)-1,2,5-thiadiazolidin-3-one 1,1-dioxide (3, 100 mg, 93.80 μmol) in DMF (2.5 mL) was added Pd/C (100 mg, 93.80 μmol, 10% purity) and Pd(OH)₂/C (100 mg, 93.80 μmol, 10% purity) under N₂ atmosphere. The suspension was degassed and purged with H₂ 3 times. The mixture was stirred at 30° C. for 16 hr under H₂ (15 psi). The reaction mixture was filtered and concentrated under vacuum. The residue was purified by prep-HPLC (flow: 25 mL/min; gradient: from 8-38% MeCN in water (0.1% TFA) over 12 min; column: Phenomenex SynergiC18 150×25 mm×10 um) to afford 3-(5-(1-(2-((R)-3-((7-(1,1-dioxido-4-oxo-1,2,5-thiadiazolidin-2-yl)-8-fluoro-6-hydroxynaphthalen-2-yl)oxy)pyrrolidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (Degrader 117, 34.27 mg, 34.27 μmol, 41% yield, TFA salt) as a white solid.

LCMS (ESI): m/z 800.1 [M+H]⁺

¹H NMR (400 MHz, DMSO-d₆) δ11.11 (s, 1H), 10.15-9.83 (m, 1H), 7.75 (br d, J=9.2 Hz, 1H), 7.42-6.80 (m, 6H), 5.44-5.16 (m, 2H), 4.52-4.26 (m, 4H), 3.93-3.82 (m, 2H), 3.77-3.65 (m, 3H), 3.55-3.26 (m, 7H), 3.23-3.07 (m, 1H), 2.98-2.86 (m, 1H), 2.76-2.66 (m, 2H), 2.41-2.26 (m, 2H), 2.24-2.16 (m, 1H), 2.14-1.90 (m, 2H). N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)-2-methylpropanamide (Degrader 118)

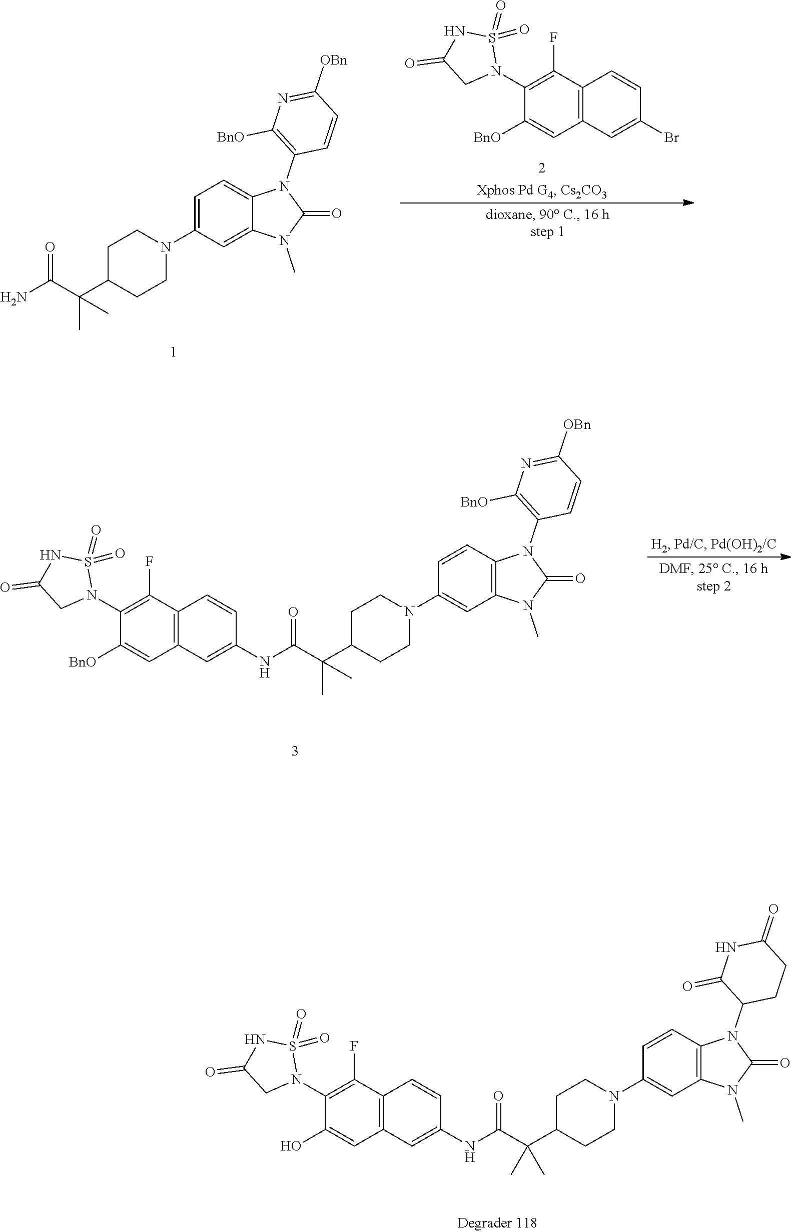 [SMILES: Cn1c(=O)n(-c2ccc(OCc3ccccc3)nc2OCc2ccccc2)c2ccc(N3CCC(C(C)(C)C(N)=O)CC3)cc21 ; O=C1CN(c2c(OCc3ccccc3)cc3cc(Br)ccc3c2F)S(=O)(=O)N1 ; Cn1c(=O)n(-c2ccc(OCc3ccccc3)nc2OCc2ccccc2)c2ccc(N3CCC(C(C)(C)C(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(N3CCC(C(C)(C)C(=O)Nc4ccc5c(F)c(N6CC(=O)NS6(=O)=O)c(O)cc5c4)CC3)cc21]

### Step 1: N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-methyl-2-oxo-2,3-dihydro-1H-benzo[d]imidazol-5-yl)piperidin-4-yl)-2-methylpropanamide (3)

To a mixture of 2-[1-[1-(2,6-dibenzyloxy-3-pyridyl)-3-methyl-2-oxo-benzimidazol-5-yl]-4-piperidyl]-2-methyl-propanamide (1, 50 mg, 82.55 μmol), 5-(3-(benzyloxy)-6-bromo-1-fluoronaphthalen-2-yl)-1,2,5-thiadiazolidin-3-one 1,1-dioxide (2, 50.0 mg, 107.46 μmol), XPhos-Pd-G4 (7.10 mg, 8.25 μmol) and Cs₂CO₃ (80.68 mg, 247.64 μmol) was added dioxane (0.5 mL). The mixture was stirred at 90° C. for 16 h under N₂. The three reaction batches were combined together. The mixture was filtered and concentrated to get a residue. The residue was purified by prep-HPLC (NH₃·H₂O) and lyophilization. The N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-methyl-2-oxo-2,3-dihydro-1H-benzo[d]imidazol-5-yl)piperidin-4-yl)-2-methylpropanamide (3, 50 mg, 49.99 μmol, 61% yield) as white solid.

LCMS (ESI): m/z 990.3 [M+H]⁺

### Step 2: N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)-2-methylpropanamide (Degrader 118)

To a solution of N-[7-benzyloxy-5-fluoro-6-(1,1,4-trioxo-1,2,5-thiadiazolidin-2-yl)-2-naphthyl]-2-[1-[1-(2,6-dibenzyloxy-3-pyridyl)-3-methyl-2-oxo-benzimidazol-5-yl]-4-piperidyl]-2-methyl-propanamide (3, 50 mg, 50.50 μmol) in DMF (2 mL) was added Pd/C (50 mg, 10% purity) and Pd(OH)₂/C (50 mg, 50.50 μmol, 10% purity) under N₂ atmosphere. The suspension was degassed and purged with H₂ 3 times. The mixture was stirred under at 30° C. for 16 hr under H₂ (15 psi). The reaction mixture was filtered and concentrated under vacuum. The residue was purified by prep-HPLC (flow: 25 mL/min; gradient: from 8-38% MeCN in water (0.1% TFA) over 12 min; column: Phenomenex SynergiC18 150×25 mm×10 um) to afford N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)-2-methylpropanamide (Degrader 118, 30.97 mg, 37.06 μmol, 74% yield, TFA salt) as a white solid.

LCMS (ESI): m/z 722.0 [M+H]⁺

¹H NMR (400 MHz, DMSO-d₆) δ=11.11 (s, 1H), 10.24-9.97 (m, 1H), 9.39 (s, 1H), 8.16 (s, 1H), 7.91-7.80 (m, 1H), 7.62 (m, 1H), 7.52-7.29 (m, 1H), 7.26-7.04 (m, 2H), 6.97 (s, 1H), 5.48-5.30 (m, 1H), 4.25 (br d, J=2.8 Hz, 2H), 3.69 (br s, 2H), 3.63 (br s, 2H), 3.35 (s, 3H), 2.95-2.86 (m, 1H), 2.77-2.62 (m, 2H), 2.09-1.95 (m, 2H), 1.91-1.79 (m, 2H), 1.78-1.65 (m, 2H), 1.27 (s, 6H).

### N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)indolin-5-yl)acetamide (Degrader 119)

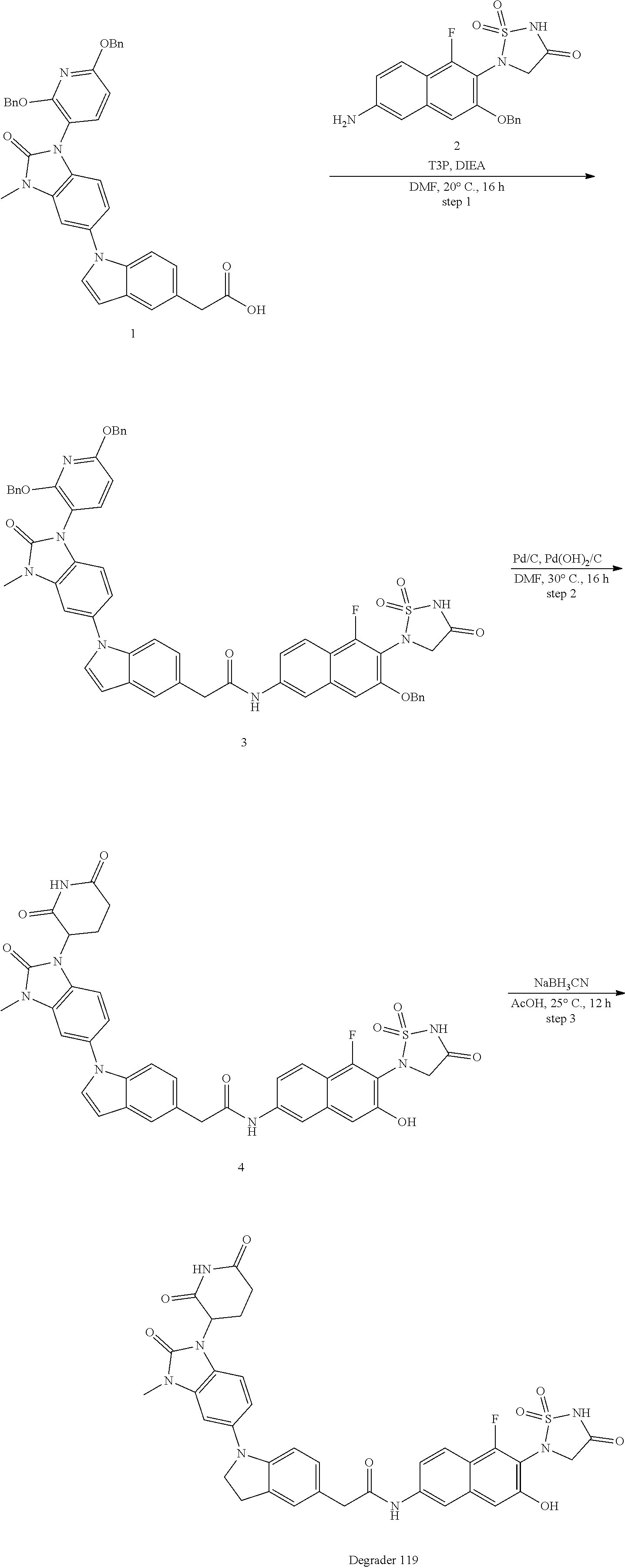

### Step 1: N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-methyl-2-oxo-2,3-dihydro-1H-benzo[d]imidazol-5-yl)-1H-indol-5-yl)acetamide (3)

To a solution of 2-[1-[1-(2,6-dibenzyloxy-3-pyridyl)-3-methyl-2-oxo-benzimidazol-5-yl]indol-5-yl]acetic acid (1, 150 mg, 245.64 μmol) and 5-(6-amino-3-(benzyloxy)-1-fluoronaphthalen-2-yl)-1,2,5-thiadiazolidin-3-one 1,1-dioxide (2, 164.59 mg, 319.33 μmol, TFA salt) in DMF (1.5 mL) were added DIPEA (158.73 mg, 1.23 mmol, 213.93 μL) and T₃P (468.94 mg, 736.91 μmol, 50% purity). The mixture was stirred at 20° C. for 16 h. The mixture was purified by Prep-HPLC (NH₃·H₂O) and then lyophilization. The N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-methyl-2-oxo-2,3-dihydro-1H-benzo[d]imidazol-5-yl)-1H-indol-5-yl)acetamide (3, 100 mg, 97.58 μmol, 40% yield) as white solid.

LCMS (ESI): m/z 995.0 [M+H]⁺

### Step 2: N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)-1H-indol-5-yl)acetamide (4)

To a solution of N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-methyl-2-oxo-2,3-dihydro-1H-benzo[d]imidazol-5-yl)-1H-indol-5-yl)acetamide (3, 50 mg, 50.30 μmol) in DMF (1 mL) was added Pd/C (50 mg, 10% purity) and Pd(OH)₂/C (50 mg, 50.30 μmol, 10% purity) under N₂ atmosphere. The suspension was degassed and purged with H₂ 3 times. The mixture was stirred at 30° C. for 16 hr under H₂ (15 psi). The reaction mixture was filtered and concentrated under vacuum. The residue was purified by prep-HPLC (flow: 25 mL/min; gradient: from 8-38% MeCN in water (0.1% TFA) over 12 min; column: Phenomenex SynergiC18 150×25 mm×10 um) to afford N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)-1H-indol-5-yl)acetamide (4, 20 mg, 23.76 μmol, 47% yield, TFA salt) as a blue solid.

LCMS (ESI): m/z 728.1 [M+H]⁺

### Step 3: N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)indolin-5-yl)acetamide (Degrader 119)

To a solution of 2-[1-[1-(2,6-dioxo-3-piperidyl)-3-methyl-2-oxo-benzimidazol-5-yl]indol-5-yl]-N-[5-fluoro-7-hydroxy-6-(1,1,4-trioxo-1,2,5-thiadiazolidin-2-yl)-2-naphthyl]acetamide (20 mg, 23.82 μmol, TFA salt) in AcOH (0.5 mL) was added sodium; cyanoboranuide (2.99 mg, 47.63 μmol). The mixture was stirred at 25° C. for 12 h. The mixture was purified by prep-HPLC (flow: 25 mL/min; gradient: from 8-38% MeCN in water (0.1% TFA) over 12 min; column: Phenomenex SynergiC18 150×25 mm×10 um) to afford N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)indolin-5-yl)acetamide (Degrader 119, 8.87 mg, 10.54 μmol, 44% yield, TFA salt) as a blue solid.

LCMS (ESI): m/z 728.0 [M+H]⁺

¹H NMR (400 MHz, DMSO-d₆) δ11.09 (s, 1H), 10.31 (s, 1H), 10.29-10.15 (m, 1H), 8.16 (s, 1H), 7.85 (d, J=8.8 Hz, 1H), 7.49-7.42 (m, 1H), 7.16 (s, 1H), 7.11-7.05 (m, 2H), 7.03-6.98 (m, 1H), 6.94 (d, J=4.8 Hz, 2H), 6.91 (s, 1H), 5.35 (m, 1H), 4.32 (s, 2H), 3.94-3.90 (m, 2H), 3.56 (s, 2H), 3.33 (s, 3H), 3.12-3.03 (m, 2H), 2.97-2.84 (m, 1H), 2.77-2.68 (m, 1H), 2.66-2.60 (m, 1H), 2.09-1.96 (m, 1H).

### N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)-1H-indol-5-yl)acetamide (Degrader 120)

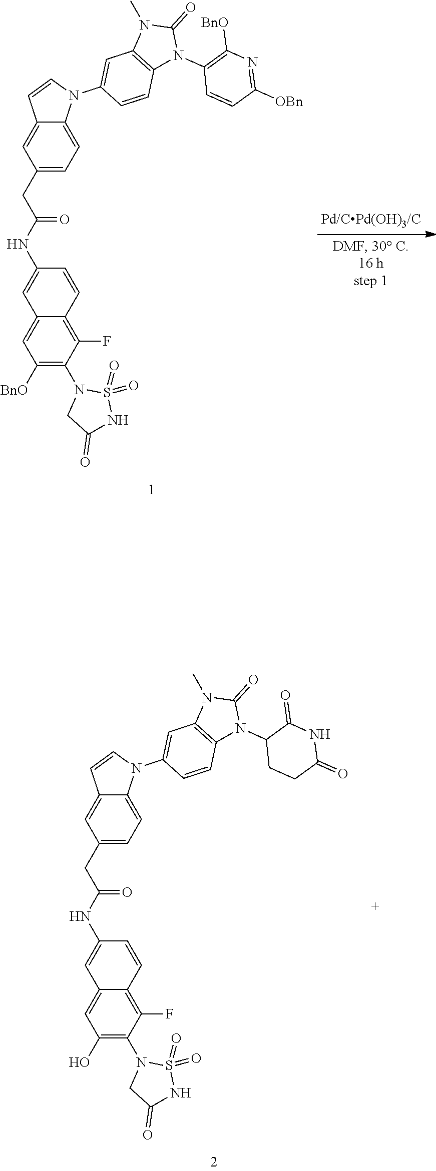 [SMILES: Cn1c(=O)n(-c2ccc(OCc3ccccc3)nc2OCc2ccccc2)c2ccc(-n3ccc4cc(CC(=O)Nc5ccc6c(F)c(N7CC(=O)NS7(=O)=O)c(OCc7ccccc7)cc6c5)ccc43)cc21 ; Cn1c(=O)n(C2CCC(=O)NC2=O)c2ccc(-n3ccc4cc(CC(=O)Nc5ccc6c(F)c(N7CC(=O)NS7(=O)=O)c(O)cc6c5)ccc43)cc21]

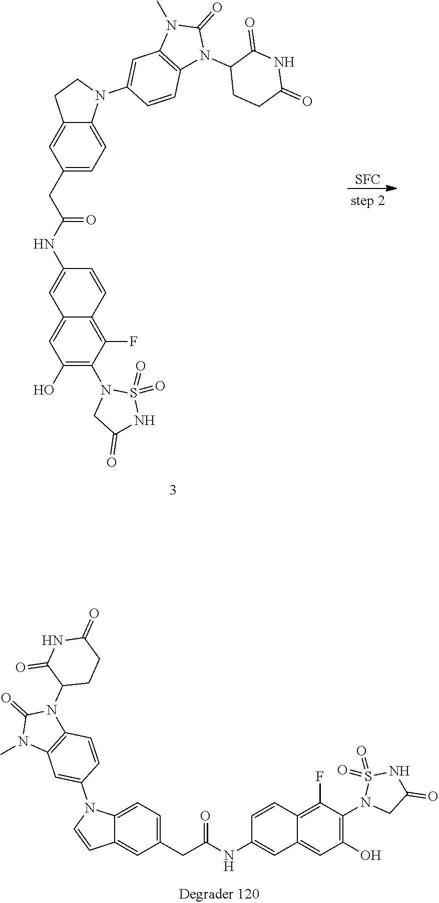 [SMILES: Cn1c(=O)n(C2CCC(=O)NC2=O)c2ccc(N3CCc4cc(CC(=O)Nc5ccc6c(F)c(N7CC(=O)NS7(=O)=O)c(O)cc6c5)ccc43)cc21 ; "SFC" ; Cn1c(=O)n(C2CCC(=O)NC2=O)c2ccc(-n3ccc4cc(CC(=O)Nc5ccc6c(F)c(N7CC(=O)NS7(=O)=O)c(O)cc6c5)ccc43)cc21]

### Step 1: N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)-1H-indol-5-yl)acetamide (2) and N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)indolin-5-yl)acetamide (3)

To a solution of N-(7-(benzyloxy)-6-(1,1-dioxido-4-oxo-1,2,5-thiadiazolidin-2-yl)-5-fluoronaphthalen-2-yl)-2-(1-(1-(2,6-bis(benzyloxy)pyridin-3-yl)-3-methyl-2-oxo-2,3-dihydro-1H-benzo[d]imidazol-5-yl)-1H-indol-5-yl)acetamide (1, 50 mg, 50.30 μmol) in DMF (1 mL) was added Pd/C (50 mg, 10% purity) and Pd(OH)₂/C (50 mg, 50.30 μmol, 10% purity) under N₂ atmosphere. The suspension was degassed and purged with H₂ 3 times. The mixture was stirred at 30° C. for 16 hr under H₂ (15 psi). The reaction mixture was filtered and concentrated under vacuum. The residue was purified by reversed phase-HPLC (FA) to afford N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)indolin-5-yl)acetamide (3, 20 mg, 27.48 μmol, 55% yield) as a blue solid. SFC data showed 37% of N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)-1H-indol-5-yl)acetamide (2) and 62% of N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)indolin-5-yl)acetamide (3).

LCMS (ESI): m/z 728.1 [M+H]⁺

### Step 2: N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)-1H-indol-5-yl)acetamide (Degrader 120)

The N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)-1H-indol-5-yl)acetamide (2, 20 mg, 27.56 μmol) & N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)indolin-5-yl)acetamide (3) was separated by SFC (Column: Chiralpak IG-3 50×4.6 mm I.D., 3 um Mobile phase: Phase A for CO₂, and Phase B for IPA+ACN (0.05% DEA); Gradient elution: 60% IPA+ACN (0.05% DEA) in CO₂ Flow rate: 3 mL/min; Detector: PDA; Column Temp: 35 C; Back Pressure: 100 Bar) and purification by prep-HPLC (flow: 25 mL/min; gradient: from 8-38% MeCN in water (0.1% TFA) over 12 min; column: Phenomenex SynergiC18 150×25 mm×10 um) to afford N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)-1H-indol-5-yl)acetamide (Degrader 120, 1.28 mg, 1.52 μmol, 6% yield, TFA salt) as white solid.

LCMS (ESI): m/z 726.0 [M+H]⁺

¹H NMR (400 MHz, DMSO-d₆) δ 11.14 (s, 1H), 10.37 (s, 1H), 9.77 (br d, J=1.6 Hz, 1H), 8.14 (s, 1H), 7.82 (d, J=9.2 Hz, 1H), 7.66-7.57 (m, 2H), 7.54-7.42 (m, 3H), 7.31-7.20 (m, 3H), 7.12 (s, 1H), 7.00 (s, 1H), 6.92 (s, 1H), 6.67 (d, J=3.2 Hz, 1H), 5.44 (m, 1H), 4.07 (s, 2H), 3.76 (s, 2H), 3.39 (br s, 2H), 3.39-3.39 (m, 1H), 3.01-2.86 (m, 1H), 2.84-2.68 (m, 2H), 2.11-2.03 (m, 1H), 1.30-1.21 (m, 1H).

### N-(6-(1,1-dioxido-4-oxo-1,2,5-thiadiazolidin-2-yl)-5-fluoro-7-hydroxynaphthalen-2-yl)-2-((2S)-4-(1-(2,6-dioxopiperidin-3-yl)-3-methyl-2-oxo-2,3-dihydro-1H-benzo[d]imidazol-5-yl)-2-methylpiperidin-1-yl)acetamide (Degrader 121)

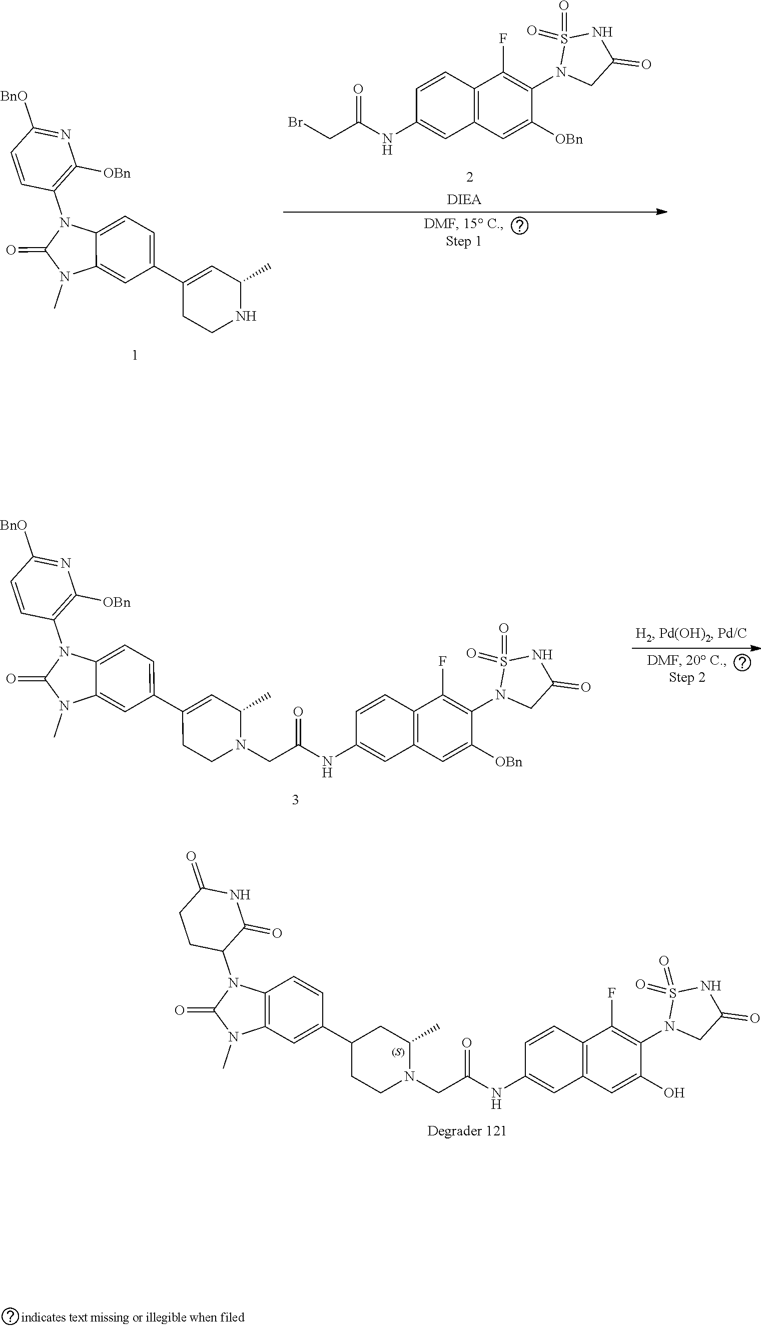

### Step 1: (S)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-2-methyl-5,6-dihydropyridin-1 (2H)-yl)acetamide (3)

To a mixture of 1-(2,6-dibenzyloxy-3-pyridyl)-3-methyl-5-[(6S)-6-methyl-1,2,3,6-tetrahydropyridin-4-yl]benzimidazol-2-one (1, 150 mg, 259.22 μmol, formic acid salt) and N-(7-(benzyloxy)-6-(1,1-dioxido-4-oxo-1,2,5-thiadiazolidin-2-yl)-5-fluoronaphthalen-2-yl)-2-bromoacetamide (2, 149 mg, 285.25 μmol) in DMF (1.5 mL) was added DIPEA (167.51 mg, 1.30 mmol, 225.76 uL). The mixture was stirred at 25° C. for 16 h. The reaction mixture was filtered. The mixture was purified by prep-HPLC (flow: 25 mL/min; gradient: from water (0.05% ammonia hydroxide v/v)-CAN over 9 min; column: Waters Xbridge 150*25 mm*5 um) to afford (S)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-2-methyl-5,6-dihydropyridin-1 (2H)-yl)acetamide (3, 110 mg, 110.67 μmol, 43% yield) as white solid.

LCMS (ESI): m/z 974.1 [M+H]⁺

### Step 2: N-(6-(1,1-dioxido-4-oxo-1,2,5-thiadiazolidin-2-yl)-5-fluoro-7-hydroxynaphthalen-2-yl)-2-((2S)-4-(1-(2,6-dioxopiperidin-3-yl)-3-methyl-2-oxo-2,3-dihydro-1H-benzo[d]imidazol-5-yl)-2-methylpiperidin-1-yl)acetamide (Degrader 121)

To a solution of N-[7-benzyloxy-5-fluoro-6-(1,1,4-trioxo-1,2,5-thiadiazolidin-2-yl)-2-naphthyl]-2-[(6S)-4-[1-(2,6-dibenzyloxy-3-pyridyl)-3-methyl-2-oxo-benzimidazol-5-yl]-6-methyl-3,6-dihydro-2H-pyridin-1-yl]acetamide (3, 110 mg, 112.93 μmol) in DMF (4 mL) was added Pd/C (110 mg, 10% purity) and Pd(OH)₂ (110 mg, 112.93 μmol, 10% purity) under N₂ atmosphere. The suspension was degassed and purged with H₂ 3 times. The mixture was stirred under H₂ (15 Psi) at 25° C. for 12 h. The reaction mixture was filtered and concentrated under vacuum. The residue was purified by prep-HPLC (flow: 25 mL/min; gradient: from 8-38% MeCN in water (0.1% TFA) over 12 min; column: Phenomenex Synergi C18 150×25 mm×10 um) to afford N-(6-(1,1-dioxido-4-oxo-1,2,5-thiadiazolidin-2-yl)-5-fluoro-7-hydroxynaphthalen-2-yl)-2-((2S)-4-(1-(2,6-dioxopiperidin-3-yl)-3-methyl-2-oxo-2,3-dihydro-1H-benzo[d]imidazol-5-yl)-2-methylpiperidin-1-yl)acetamide (Degrader 121, 44.82 mg, 54.54 μmol, 48% yield, TFA salt) as a white solid

LCMS (ESI): m/z 708.2 [M+H]⁺

¹H NMR (400 MHz, DMSO-d₆) δ11.11 (s, 1H), 10.91-10.76 (m, 1H), 10.22-9.88 (m, 1H), 9.86-9.46 (m, 1H), 8.17-8.09 (m, 1H), 7.99-7.85 (m, 1H), 7.56-7.44 (m, 1H), 7.19-7.04 (m, 2H), 7.02 (s, 1H), 6.99-6.90 (m, 1H), 5.36 (m, 1H), 4.60-4.46 (m, 1H), 4.33-4.12 (m, 4H), 4.10-3.96 (m, 2H), 3.81-3.72 (m, 1H), 3.69-3.58 (m, 1H), 3.09-2.84 (m, 2H), 2.77-2.57 (m, 3H), 2.12-1.91 (m, 5H), 1.48-1.27 (m, 3H).

### 2-[4-[3-(2,6-dioxo-3-piperidyl)-1-methyl-2-oxo-imidazo[4,5-c]pyridin-6-yl]phenyl]-N-[5-fluoro-7-hydroxy-6-(1,1,4-trioxo-1,2,5-thiadiazolidin-2-yl)-2-naphthyl]acetamide (Degrader 122)

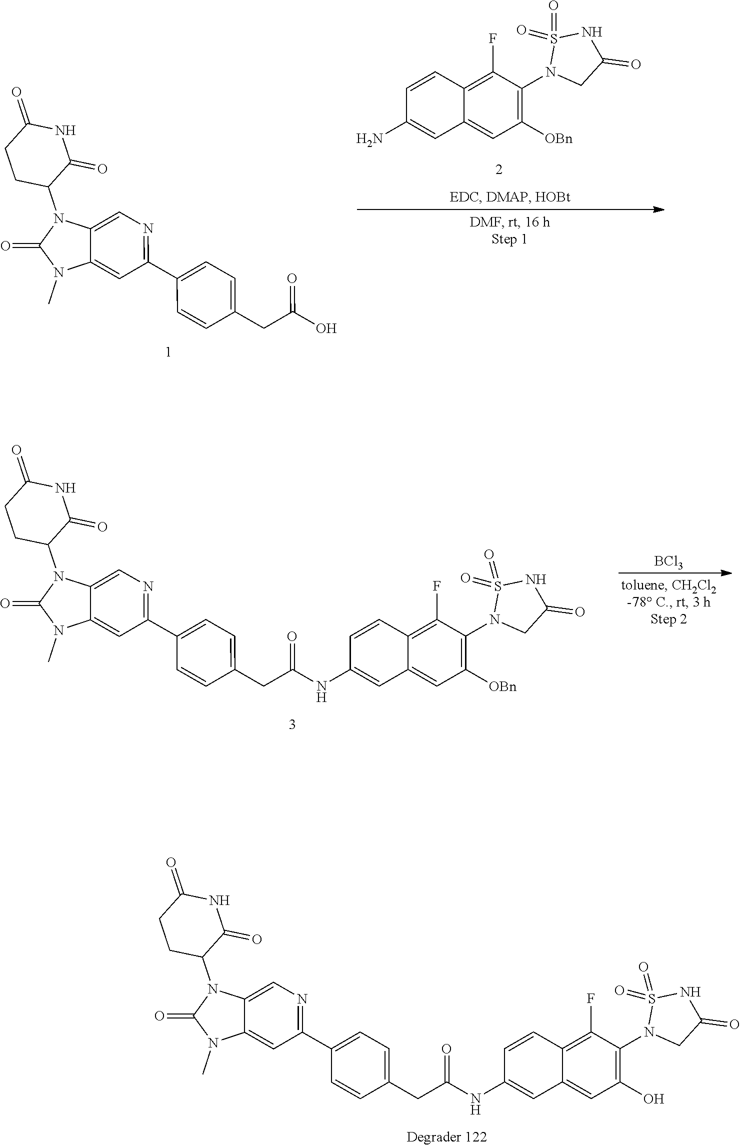 [SMILES: Cn1c(=O)n(C2CCC(=O)NC2=O)c2cnc(-c3ccc(CC(=O)O)cc3)cc21 ; Nc1ccc2c(F)c(N3CC(=O)NS3(=O)=O)c(OCc3ccccc3)cc2c1 ; Cn1c(=O)n(C2CCC(=O)NC2=O)c2cnc(-c3ccc(CC(=O)Nc4ccc5c(F)c(N6CC(=O)NS6(=O)=O)c(OCc6ccccc6)cc5c4)cc3)cc21 ; "BCl3" ; Cn1c(=O)n(C2CCC(=O)NC2=O)c2cnc(-c3ccc(CC(=O)Nc4ccc5c(F)c(N6CC(=O)NS6(=O)=O)c(O)cc5c4)cc3)cc21]

### Step 1: N-[7-benzyloxy-5-fluoro-6-(1,1,4-trioxo-1,2,5-thiadiazolidin-2-yl)-2-naphthyl]-2-[4-[3-(2,6-dioxo-3-piperidyl)-1-methyl-2-oxo-imidazo[4,5-c]pyridin-6-yl]phenyl]acetamide (10)

Into a 25 mL single-neck round-bottom flask containing a well-stirred solution of 2-[4-[3-(2,6-dioxo-3-piperidyl)-1-methyl-2-oxo-imidazo[4,5-c]pyridin-6-yl]phenyl]acetic acid (1, 180 mg, 360.36 μmol, formic acid salt) and 5-(6-amino-3-benzyloxy-1-fluoro-2-naphthyl)-1,1-dioxo-1,2,5-thiadiazolidin-3-one (2, 191.07 mg, 360.36 μmol, TFA salt) in dry DMF (5 mL) was added EDC·HCl (206.49 mg, 1.08 mmol), HOBT (97.39 mg, 720.73 μmol) and DMAP (264.15 mg, 2.16 mmol) at room temperature and the resulting mixture was stirred for 16 h at room temperature. The reaction mixture was concentrated under reduced pressure and diluted with ice-cold water to precipitate a solid that was filtered and dried to afford N-[7-benzyloxy-5-fluoro-6-(1,1,4-trioxo-1,2,5-thiadiazolidin-2-yl)-2-naphthyl]-2-[4-[3-(2,6-dioxo-3-piperidyl)-1-methyl-2-oxo-imidazo[4,5-c]pyridin-6-yl]phenyl]acetamide (3, 230 mg, 204.13 μmol, 57% yield) as a pale brown solid.

LCMS (ES+): m/z 778.0 [M+H]⁺

### Step 2: 2-[4-[3-(2,6-dioxo-3-piperidyl)-1-methyl-2-oxo-imidazo[4,5-c]pyridin-6-yl]phenyl]-N-[5-fluoro-7-hydroxy-6-(1,1,4-trioxo-1,2,5-thiadiazolidin-2-yl)-2-naphthyl]acetamide (Degrader 122)

Into a 50 mL single-neck round-bottom flask containing well-stirred solution of N-[7-benzyloxy-5-fluoro-6-(1,1,4-trioxo-1,2,5-thiadiazolidin-2-yl)-2-naphthyl]-2-[4-[3-(2,6-dioxo-3-piperidyl)-1-methyl-2-oxo-imidazo[4,5-c]pyridin-6-yl]phenyl]acetamide (3, 330 mg, 292.89 μmol) in dry toluene (6 mL) and dry DCM (6 mL) was added pent methylbenzene (217.09 mg, 1.46 mmol) under nitrogen atmosphere at room temperature. The reaction mixture was then cooled to −78° C. and BCl₃ solution (1.0 M in methylene chloride, 4.39 mmol, 4.39 mL) was added dropwise. Subsequently, the reaction mixture was stirred at room temperature for 3 h. Afterwards, reaction mixture was quenched with 5% MeOH in DCM (4 mL) at −78° C. and concentrated under reduced pressure to get the crude material that was purified by reverse-phase preparative HPLC [Column: Redisep gold C18, 19×150 mm, 5 μm; Mobile phase A: 0.1% TFA in water and Mobile phase B: MeCN] to afford 2-[4-[3-(2,6-dioxo-3-piperidyl)-1-methyl-2-oxo-imidazo[4,5-c]pyridin-6-yl]phenyl]-N-[5-fluoro-7-hydroxy-6-(1,1,4-trioxo-1,2,5-thiadiazolidin-2-yl)-2-naphthyl]acetamide (Degrader 122, 61 mg, 65.16 μmol, 22% yield, TFA salt) as a pale brown solid.

LCMS (ES+): m/z 688.1 [M+H]⁺

¹H-NMR (400 MHz, DMSO-d6): δ 11.20 (s, 1H), 10.49 (s, 1H), 10.07 (brs, 1H), 8.53 (s, 1H), 8.16 (s, 1H), 8.05-7.98 (m, 3H), 7.86 (d, J=8.80 Hz, 1H), 7.55-7.46 (m, 3H), 6.96 (s, 1H), 5.53 (dd, J=4.80, 12.80 Hz, 1H), 4.23 (s, 2H), 3.81 (s, 2H), 3.49 (s, 3H), 2.99-2.65 (m, 3H), 2.14-2.08 (m, 1H).

### 2-[(3R)-3-[1-(2,6-dioxo-3-piperidyl)-3-methyl-2-oxo-benzimidazol-5-yl]pyrrolidin-1-yl]-N-[5-fluoro-7-hydroxy-6-(1,1,4-trioxo-1,2,5-thiadiazolidin-2-yl)-2-naphthyl]acetamide (Degrader 123)

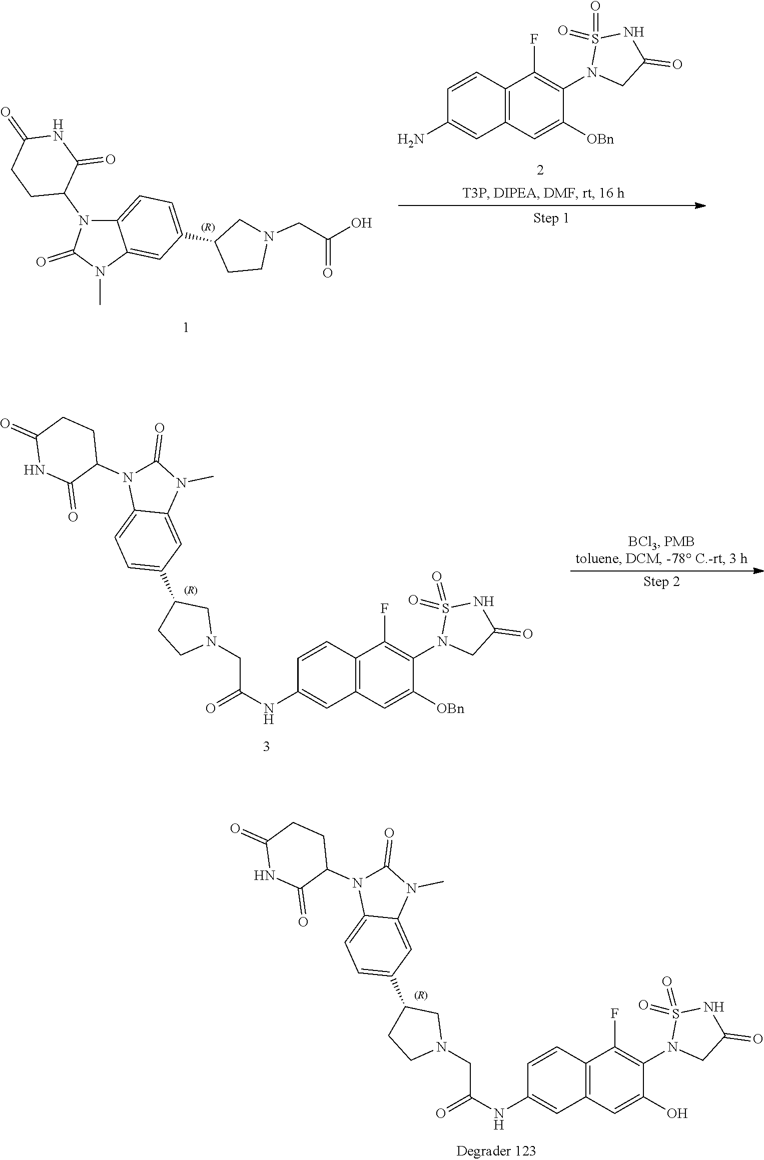

Note: Configurations are arbitrarily assigned.

### Step 1: N-[7-benzyloxy-5-fluoro-6-(1,1,4-trioxo-1,2,5-thiadiazolidin-2-yl)-2-naphthyl]-2-[(3R)-3-[1-(2,6-dioxo-3-piperidyl)-3-methyl-2-oxo-benzimidazol-5-yl]pyrrolidin-1-yl]acetamide (3)

Into a 50 mL single neck round bottom flask containing a well-stirred solution of 2-[(3R)-3-[1-(2,6-dioxo-3-piperidyl)-3-methyl-2-oxo-benzimidazol-5-yl]pyrrolidin-1-yl]acetic acid (1, 170 mg, 336.31 μmol, TFA salt) in DMF (4 mL) were added DIPEA (217.33 mg, 1.68 mmol, 292.89 μL) and propylphosphonic anhydride solution (>50 wt. % in ethyl acetate) (428.06 mg, 672.63 μmol, 50% purity) at room temperature. Then 5-(6-amino-3-benzyloxy-1-fluoro-2-naphthyl)-1,1-dioxo-1,2,5-thiadiazolidin-3-one (2, 178.71 mg, 336.31 μmol, TFA salt). After 16 h, the reaction mixture was concentrated under reduced pressure and the residue was treated with 1.5N HCl (5 mL) at 0° C. The precipitate was filtered, washed with cold water (5 mL) and diethyl ether (5 mL) to afford N-[7-benzyloxy-5-fluoro-6-(1,1,4-trioxo-1,2,5-thiadiazolidin-2-yl)-2-naphthyl]-2-[(3R)-3-[1-(2,6-dioxo-3-piperidyl)-3-methyl-2-oxo-benzimidazol-5-yl]pyrrolidin-1-yl]acetamide (3, 210 mg, 178.42 μmol, 53% yield, HCl salt) as a pale yellow solid.

LCMS (ES+): m/z 770.0 [M+H]⁺

### Step 2: 2-[(3R)-3-[1-(2,6-dioxo-3-piperidyl)-3-methyl-2-oxo-benzimidazol-5-yl]pyrrolidin-1-yl]-N-[5-fluoro-7-hydroxy-6-(1,1,4-trioxo-1,2,5-thiadiazolidin-2-yl)-2-naphthyl]acetamide (Degrader 123)

Into a 50 mL single neck round bottom flask containing a well-stirred solution of N-[7-benzyloxy-5-fluoro-6-(1,1,4-trioxo-1,2,5-thiadiazolidin-2-yl)-2-naphthyl]-2-[(3R)-3-[1-(2,6-dioxo-3-piperidyl)-3-methyl-2-oxo-benzimidazol-5-yl]pyrrolidin-1-yl]acetamide (3, 180 mg, 159.00 μmol) in anhydrous DCM (4 mL) and toluene (4 mL) was added pentamethylbenzene (117.86 mg, 795.02 μmol) at room temperature. Then a 1.0 M solution of boron trichloride in dichloromethane (3.18 mmol, 3.18 mL) was added at −75° C. The reaction mixture was stirred at room temperature. After 3 h, the reaction mixture was quenched with 5% MeOH in DCM (2 mL) at −75° C. The solvent was removed under reduced pressure and the residue was washed with diethyl ether (10 mL). The crude compound was purified by reverse phase prep HPLC [Purification method: Column: Xselect, C18 (250×19) mm; 5 micron; Mobile phase A: 0.1% TFA in water; Mobile phase B: MeCN] to afford 2-[(3R)-3-[1-(2,6-dioxo-3-piperidyl)-3-methyl-2-oxo-benzimidazol-5-yl]pyrrolidin-1-yl]-N-[5-fluoro-7-hydroxy-6-(1,1,4-trioxo-1,2,5-thiadiazolidin-2-yl)-2-naphthyl]acetamide (Degrader 123, 90 mg, 108.10 μmol, 68% yield, TFA salt) as an off-white solid.

LCMS (ES+): m/z 680.2 [M+H]⁺

¹H-NMR (400 MHz, DMSO-d6): δ 11.11 (s, 1H), 10.78 (s, 1H), 10.40 (s, 1H), 10.03 (s, 1H), 8.12 (s, 1H), 7.92 (d, J=9.20 Hz, 1H), 7.48 (d, J=9.20 Hz, 1H), 7.24 (d, J=14.80 Hz, 1H), 7.13-7.01 (m, 3H), 5.40-5.36 (m, 1H), 4.42 (s, 2H), 4.19 (s, 2H), 4.04-3.77 (m, 3H), 3.37 (s, 3H), 2.95-2.87 (m, 1H), 2.78-2.61 (m, 3H), 2.25-2.09 (m, 1H), 2.03-2.00 (m, 1H).

### 2-[(3S)-3-[1-(2,6-dioxo-3-piperidyl)-3-methyl-2-oxo-benzimidazol-5-yl]pyrrolidin-1-yl]-N-[5-fluoro-7-hydroxy-6-(1,1,4-trioxo-1,2,5-thiadiazolidin-2-yl)-2-naphthyl]acetamide (Degrader 124)

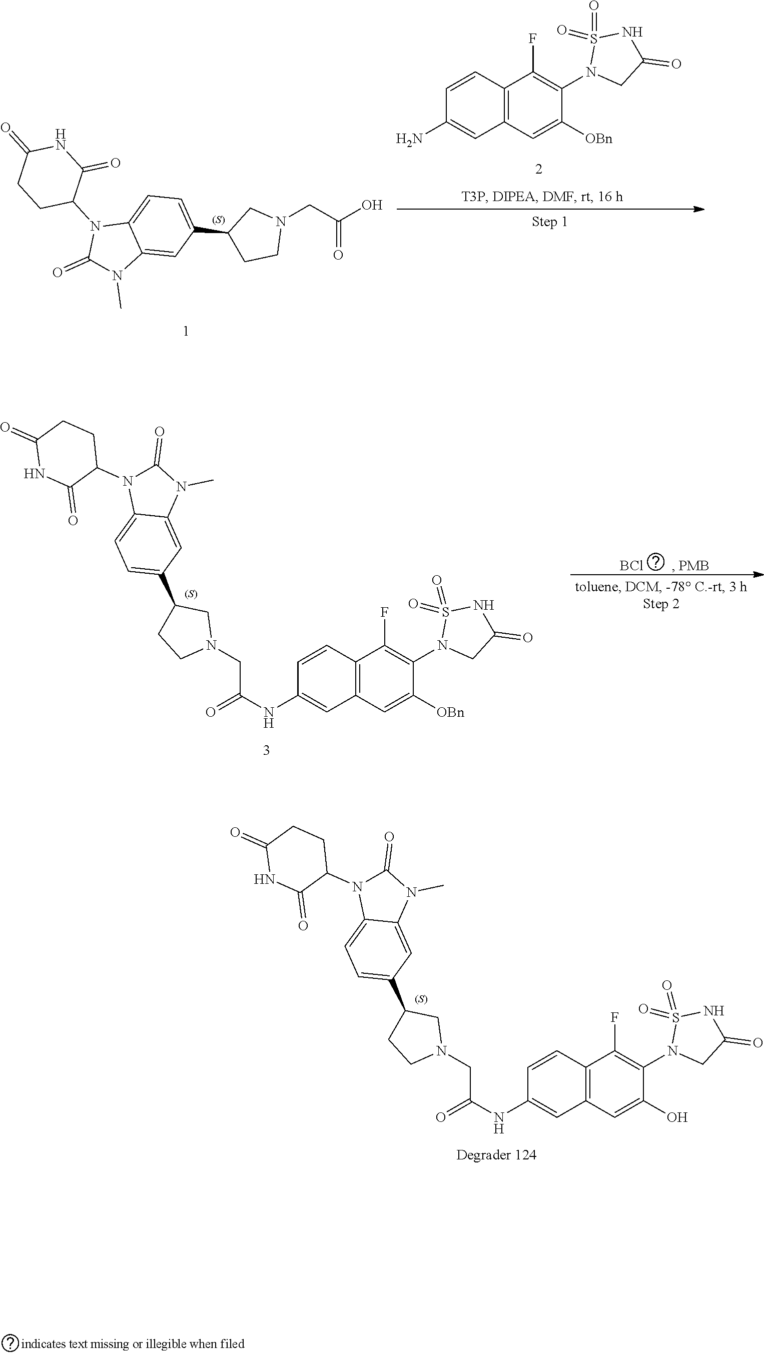

Note: Configurations are arbitrarily assigned.

2-[(3 S)-3-[1-(2,6-dioxo-3-piperidyl)-3-methyl-2-oxo-benzimidazol-5-yl]pyrrolidin-1-yl]-N-[5-fluoro-7-hydroxy-6-(1,1,4-trioxo-1,2,5-thiadiazolidin-2-yl)-2-naphthyl]acetamide (Degrader 124 100 mg, 122.01 μmol, TFA salt) was synthesized in two steps following the same procedure as 2-[(3R)-3-[1-(2,6-dioxo-3-piperidyl)-3-methyl-2-oxo-benzimidazol-5-yl]pyrrolidin-1-yl]-N-[5-fluoro-7-hydroxy-6-(1,1,4-trioxo-1,2,5-thiadiazolidin-2-yl)-2-naphthyl]acetamide (Degrader 123).

LCMS (ES+): m/z 680.2 [M+H]⁺

1H-NMR (400 MHz, DMSO-d6): δ 11.11 (s, 1H), 10.78 (s, 1H), 10.40 (s, 1H), 10.01 (s, 1H), 8.12 (s, 1H), 7.92 (d, J=8.80 Hz, 1H), 7.48 (d, J=8.80 Hz, 1H), 7.24 (d, J=15.20 Hz, 1H), 7.13-7.09 (m, 3H), 5.40-5.36 (m, 1H), 4.42 (s, 2H), 4.17 (s, 2H), 4.04-3.59 (m, 4H), 3.37 (s, 3H), 2.92-2.87 (m, 1H), 2.74-2.51 (m, 3H), 2.17-2.00 (m, 2H).

LCMS (ES+): m/z 387.2 [M+H]⁺

### 2-[4-[3-(2,4-dioxohexahydropyrimidin-1-yl)-5-fluoro-1-methyl-indazol-6-yl]phenyl]-N-[5-fluoro-7-hydroxy-6-(1,1,4-trioxo-1,2,5-thiadiazolidin-2-yl)-2-naphthyl]acetamide (Degrader 125)

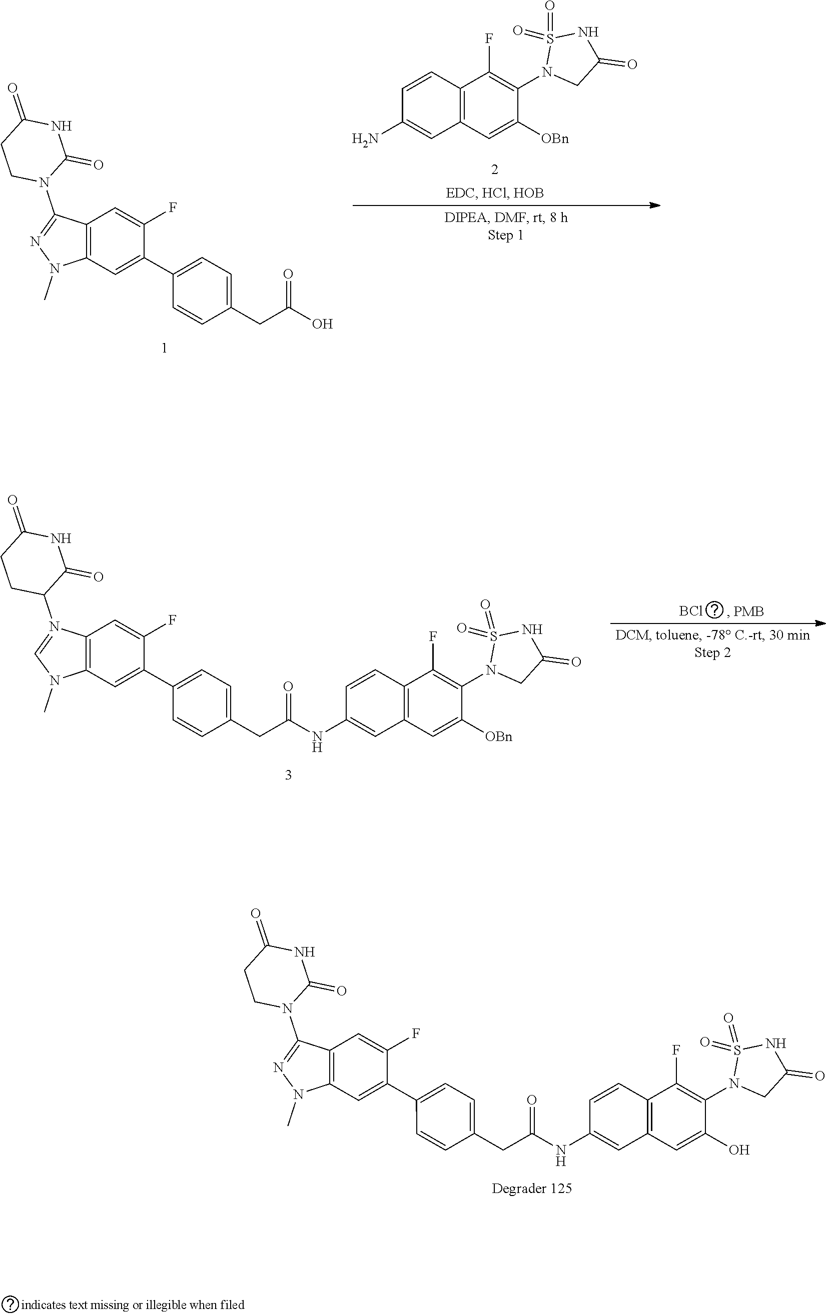

### Step 1: N-[7-benzyloxy-5-fluoro-6-(1,1,4-trioxo-1,2,5-thiadiazolidin-2-yl)-2-naphthyl]-2-[4-[3-(2,4-dioxohexahydropyrimidin-1-yl)-5-fluoro-1-methyl-indazol-6-yl]phenyl]acetamide (3)

Into a 25 mL single-neck round-bottom flask containing well-stirred solution of 2-[4-[3-(2,4-dioxohexahydropyrimidin-1-yl)-5-fluoro-1-methyl-indazol-6-yl]phenyl]acetic acid (1, 120 mg, 293.63 μmol) and 5-(6-amino-3-benzyloxy-1-fluoro-2-naphthyl)-1,1-dioxo-1,2,5-thiadiazolidin-3-one (2, 132.42 mg, 228.68 μmol, TFA salt) in anhydrous DMF (5 mL) were added EDC·HCl (225.16 mg, 1.17 mmol), DMAP (251.11 mg, 2.06 mmol) and HOBT (119.03 mg, 880.90 μmol) at room temperature. The resultant mixture was stirred at room temperature for 8 h. The solvent was evaporated and the residue was treated with 1.5 N HCl (70 mL). The precipitated solid was filtered and dried under reduced pressure to get N-[7-benzyloxy-5-fluoro-6-(1,1,4-trioxo-1,2,5-thiadiazolidin-2-yl)-2-naphthyl]-2-[4-[3-(2,4-dioxohexahydropyrimidin-1-yl)-5-fluoro-1-methyl-indazol-6-yl]phenyl]acetamide (3, 160 mg, 178.51 μmol, 61% yield) as off white solid.

UPLC-MS (ES+): m/z 780.8 [M+H]⁺

### Step 2: 2-[4-[3-(2,4-dioxohexahydropyrimidin-1-yl)-5-fluoro-1-methyl-indazol-6-yl]phenyl]-N-[5-fluoro-7-hydroxy-6-(1,1,4-trioxo-1,2,5-thiadiazolidin-2-yl)-2-naphthyl]acetamide (Degrader 125)

Into a 25 mL single-neck round-bottom flask containing well-stirred solution of N-[7-benzyloxy-5-fluoro-6-(1,1,4-trioxo-1,2,5-thiadiazolidin-2-yl)-2-naphthyl]-2-[4-[3-(2,4-dioxohexahydropyrimidin-1-yl)-5-fluoro-1-methyl-indazol-6-yl]phenyl]acetamide (3, 160 mg, 178.51 μmol) and pentamethylbenzene (132.32 mg, 892.57 μmol) in anhydrous DCM (4 mL) and toluene (4 mL) was added boron trichloride (1.0 M solution in DCM, 2.68 mmol, 2.68 mL) at −78° C. and the resultant mixture was stirred at room temperature for 30 min. The reaction mixture was quenched with 5% MeOH in DCM (3 mL) at −78° C. Subsequently, the reaction mixture was concentrated under reduced pressure to get the crude compound that was purified by reverse-phase preparative HPLC [Column: X-Select C18 (150×19) mm, 5 μm; Mobile phase A: 0.1% TFA in water and Mobile Phase B: MeCN] to get 2-[4-[3-(2,4-dioxohexahydropyrimidin-1-yl)-5-fluoro-1-methyl-indazol-6-yl]phenyl]-N-[5-fluoro-7-hydroxy-6-(1,1,4-trioxo-1,2,5-thiadiazolidin-2-yl)-2-naphthyl]acetamide (Degrader 125, 45 mg, 54.44 μmol, 31% yield, TFA salt) as an off-white solid.

¹H NMR (400 MHz, DMSO) δ 10.59 (s, 1H), 10.48 (s, 2H), 8.21-8.16 (m, 1H), 7.88 (d, J=9.0 Hz, 1H), 7.78 (d, J=6.4 Hz, 1H), 7.60 (dd, J=8.3, 1.9 Hz, 2H), 7.56-7.49 (m, 4H), 7.47 (dd, J=9.1, 2.0 Hz, 1H), 6.96 (s, 1H), 4.41 (s, 2H), 4.03 (s, 3H), 3.94 (t, J=6.7 Hz, 2H), 3.79 (s, 2H), 2.77 (t, J=6.7 Hz, 2H).

LCMS (ES+): m/z 690.0 [M+H]⁺

### 2-[(3R,4S)-1-[1-(2,6-dioxo-3-piperidyl)-3-methyl-2-oxo-benzimidazol-5-yl]-3-methyl-4-piperidyl]-N-[5-fluoro-7-hydroxy-6-(1,1,4-trioxo-1,2,5-thiadiazolidin-2-yl)-2-naphthyl]acetamide (Degrader 126)

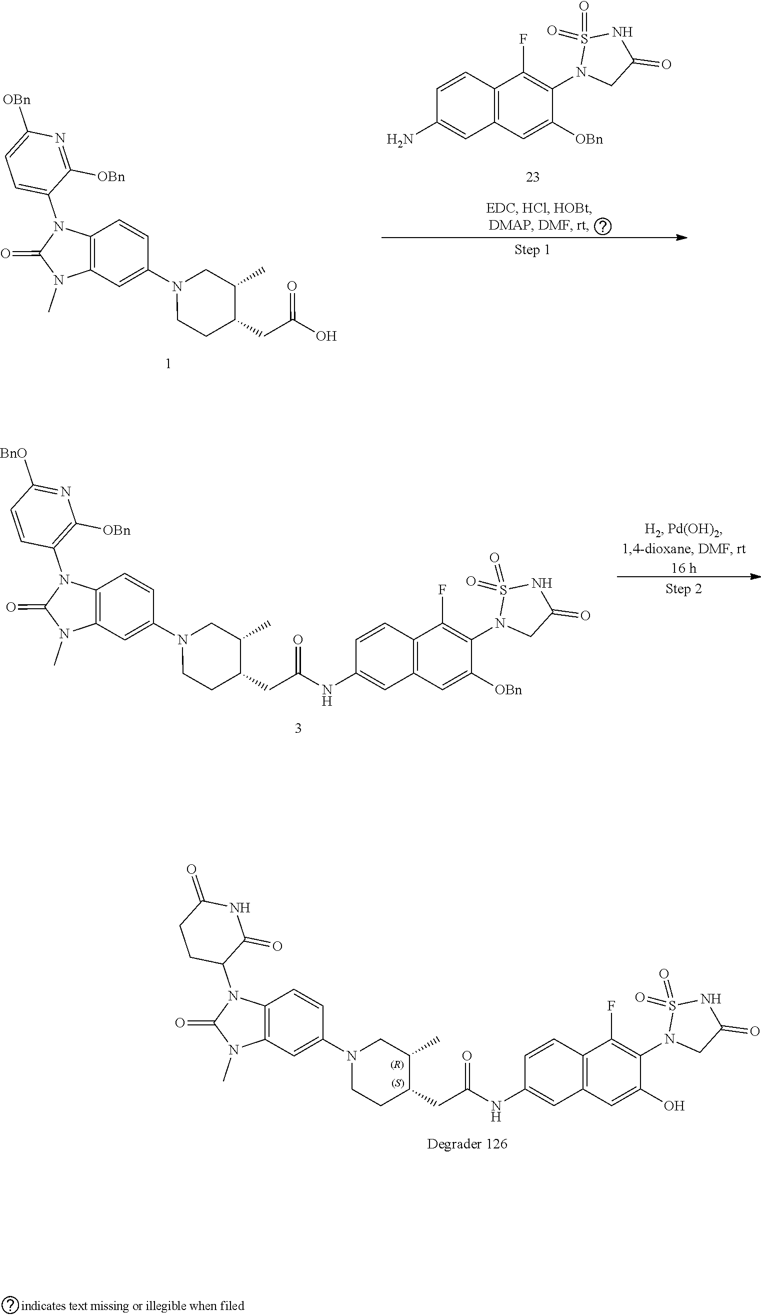

Note: Configurations are arbitrarily assigned.

### Step 1: N-[7-benzyloxy-5-fluoro-6-(1,1,4-trioxo-1,2,5-thiadiazolidin-2-yl)-2-naphthyl]-2-[(3R,4S)-1-[1-(2,6-dibenzyloxy-3-pyridyl)-3-methyl-2-oxo-benzimidazol-5-yl]-3-methyl-4-piperidyl]acetamide (3)

Into a 20 mL vial containing a solution of 2-[(3R,4S)-1-[1-(2,6-dibenzyloxy-3-pyridyl)-3-methyl-2-oxo-benzimidazol-5-yl]-3-methyl-4-piperidyl]acetic acid (1, 250 mg, 408.73 μmol) and 5-(6-amino-3-benzyloxy-1-fluoro-2-naphthyl)-1,1-dioxo-1,2,5-thiadiazolidin-3-one (2, 169.12 mg, 318.27 μmol, HCl salt) in DMF (5 mL) were added N-(3-dimethylaminopropyl)-N′-ethylcarbodiimide hydrochloride (391.77 mg, 2.04 mmol), 4-(dimethylamino)pyridine (499.35 mg, 4.09 mmol) and HOBt (165.68 mg, 1.23 mmol). The resulting mixture was stirred at ambient temperature. After 16 h, the solvent was removed under vacuum and the residue was treated with aq. 1.5 N HCl (5 mL). The solid precipitated was collected by filtration, dried under vacuum. The crude compound was purified by reverse phase column chromatography [Biotage, 120 g of C18 column; Mobile phase A: 0.1 mM ammonium bicarbonate in water; Mobile phase B: acetonitrile] to get N-[7-benzyloxy-5-fluoro-6-(1,1,4-trioxo-1,2,5-thiadiazolidin-2-yl)-2-naphthyl]-2-[(3R,4S)-1-[1-(2,6-dibenzyloxy-3-pyridyl)-3-methyl-2-oxo-benzimidazol-5-yl]-3-methyl-4-piperidyl]acetamide (3, 210 mg, 209.12 μmol, 51% yield) as a light brown solid.

LCMS (ES+): m/z 976.2 [M+H]⁺

### Step 2: 2-[(3R,4S)-1-[1-(2,6-dioxo-3-piperidyl)-3-methyl-2-oxo-benzimidazol-5-yl]-3-methyl-4-piperidyl]-N-[5-fluoro-7-hydroxy-6-(1,1,4-trioxo-1,2,5-thiadiazolidin-2-yl)-2-naphthyl]acetamide (Degrader 126)

Into a 50 mL single-neck round bottom flask containing a well-stirred solution of N-[7-benzyloxy-5-fluoro-6-(1,1,4-trioxo-1,2,5-thiadiazolidin-2-yl)-2-naphthyl]-2-[(3R,4S)-1-[1-(2,6-dibenzyloxy-3-pyridyl)-3-methyl-2-oxo-benzimidazol-5-yl]-3-methyl-4-piperidyl]acetamide (3, 190 mg, 188.82 μmol) in anhydrous 1,4-dioxane (6 mL) and DMF (2 mL) was added 20 wt. % palladium hydroxide on carbon (331.44 mg, 472.04 μmol, 20% purity). The resultant reaction mixture was stirred at room temperature under hydrogen bladder pressure for 16 h. After completion of starting material, reaction mixture was filtered, washed with mixture of 1,4-dioxane (20 mL) and DMF (7 mL). The filtrate was evaporated under reduced pressure. The crude compound was purified by reverse phase prep HPLC [Purification method: XSelect C18 (250×19) mm, 5 microns; Mobile phase A: 0.1% formic acid in water; Mobile phase B: Acetonitrile] to afford 2-[(3R,4S)-1-[1-(2,6-dioxo-3-piperidyl)-3-methyl-2-oxo-benzimidazol-5-yl]-3-methyl-4-piperidyl]-N-[5-fluoro-7-hydroxy-6-(1,1,4-trioxo-1,2,5-thiadiazolidin-2-yl)-2-naphthyl]acetamide (Degrader 126, 36 mg, 50.70 μmol, 27% yield) as an off-white solid.

LCMS (ES+): m/z 708.2 [M+H]⁺

¹H NMR (400 MHz, DMSO-d6): δ 11.12 (s, 1H), 10.25 (bs, 2H), 8.19 (s, 1H), 7.86 (d, J=8.80 Hz, 1H), 7.45 (dd, J=1.60, 9.00 Hz, 1H), 7.24 (s, 1H), 7.13-7.11 (m, 1H), 7.01-6.97 (m, 2H), 5.38-5.34 (m, 1H), 4.30 (s, 2H), 3.33-3.17 (m, 9H), 2.94-2.86 (m, 1H), 2.76-2.61 (m, 2H), 2.51-2.50 (m, 2H), 2.47-2.33 (m, 1H), 2.18-2.00 (m, 1H), 1.86-1.76 (m, 2H), 1.08 (d, J=6.80 Hz, 3H).

### 2-[(3S,4R)-1-[1-(2,6-dioxo-3-piperidyl)-3-methyl-2-oxo-benzimidazol-5-yl]-3-methyl-4-piperidyl]-N-[5-fluoro-7-hydroxy-6-(1,1,4-trioxo-1,2,5-thiadiazolidin-2-yl)-2-naphthyl]acetamide (Degrader 127)

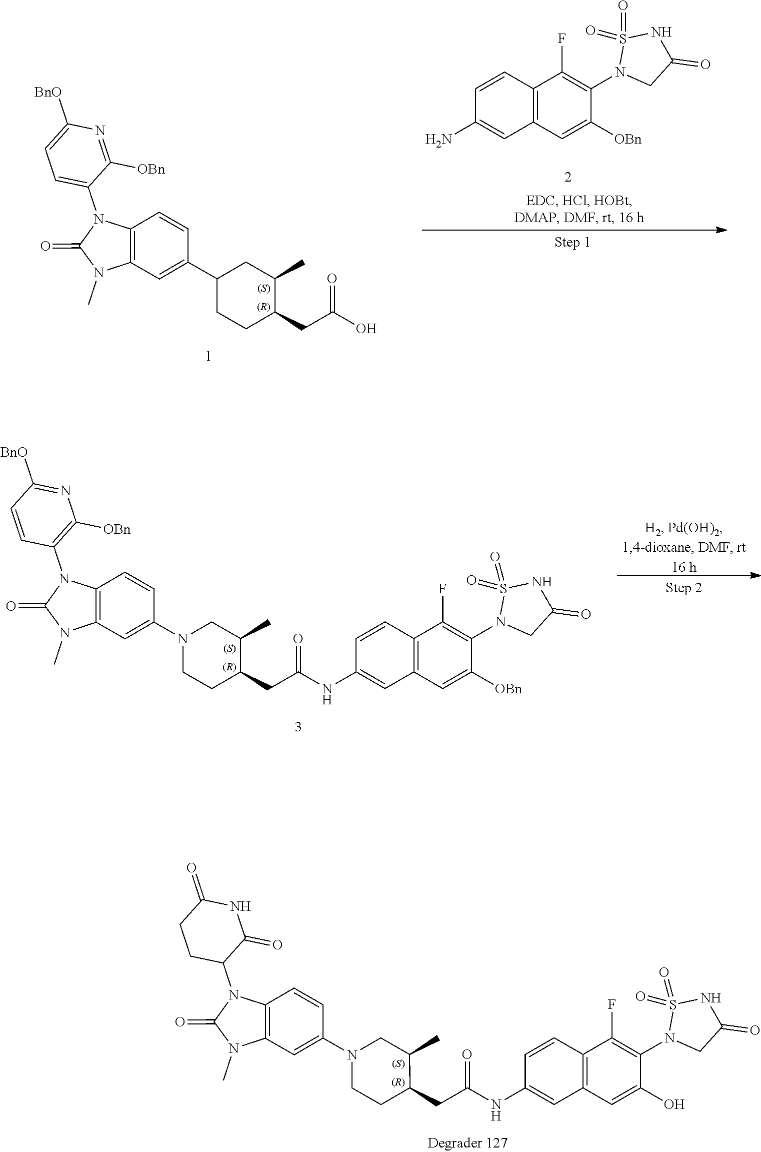

2-[(3S,4R)-1-[1-(2,6-dioxo-3-piperidyl)-3-methyl-2-oxo-benzimidazol-5-yl]-3-methyl-4-piperidyl]-N-[5-fluoro-7-hydroxy-6-(1,1,4-trioxo-1,2,5-thiadiazolidin-2-yl)-2-naphthyl]acetamide (Degrader 127, 32 mg, 44.82 μmol) was synthesized following the same two step procedure as 2-[(3R,4S)-1-[1-(2,6-dioxo-3-piperidyl)-3-methyl-2-oxo-benzimidazol-5-yl]-3-methyl-4-piperidyl]-N-[5-fluoro-7-hydroxy-6-(1,1,4-trioxo-1,2,5-thiadiazolidin-2-yl)-2-naphthyl]acetamide (Degrader 126)

LCMS (ES−): m/z 706.2 [M−H]⁻

¹H NMR (400 MHz, DMSO) δ 11.08 (s, 1H), 10.19 (s, 2H), 8.23-8.13 (m, 1H), 7.90-7.79 (m, 1H), 7.44 (dd, J=9.0, 2.0 Hz, 1H), 7.07 (s, 1H), 6.96 (s, 1H), 5.36-5.25 (m, 1H), 4.30 (s, 2H), 3.34 (s, 3H), 2.97-2.84 (m, 1H), 2.75-2.54 (m, 3H), 2.46-2.39 (m, 2H), 2.31-2.22 (m, 1H), 2.19-2.07 (m, 1H), 2.05-1.96 (m, 1H), 1.83-1.60 (m, 2H), 1.14-0.99 (m, 3H). Additional protons under solvent peaks.

### 2-[4-[4-(2,6-dioxo-3-piperidyl)phenyl]-1-piperidyl]-N-[5-fluoro-7-hydroxy-6-(1,1,4-trioxo-1,2,5-thiadiazolidin-2-yl)-2-naphthyl]acetamide (Degrader 128)

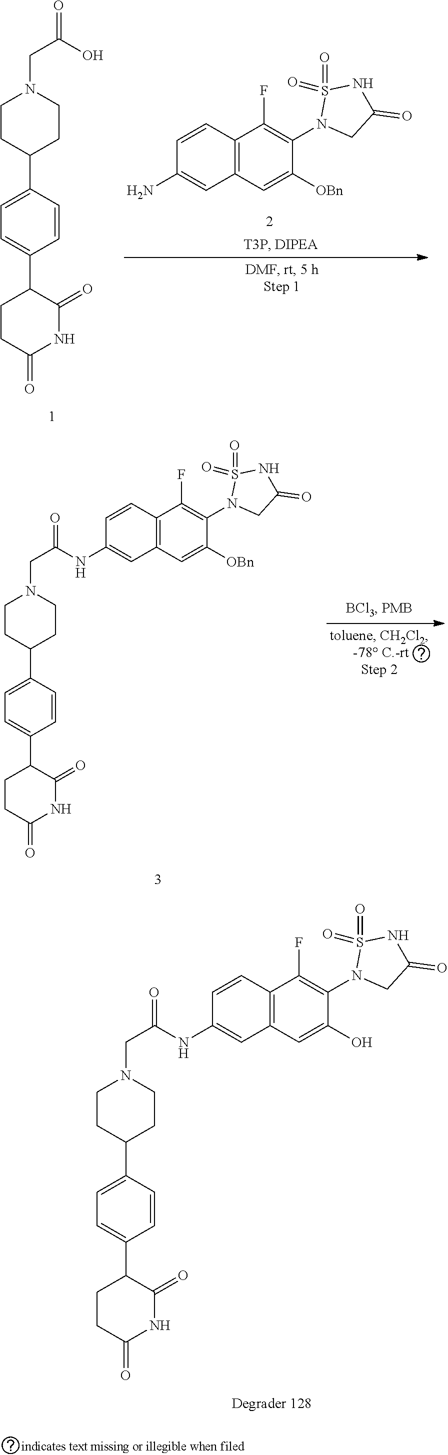 [SMILES: O=C(O)CN1CCC(c2ccc(C3CCC(=O)NC3=O)cc2)CC1 ; Nc1ccc2c(F)c(N3CC(=O)NS3(=O)=O)c(OCc3ccccc3)cc2c1 ; O=C1CCC(c2ccc(C3CCN(CC(=O)Nc4ccc5c(F)c(N6CC(=O)NS6(=O)=O)c(OCc6ccccc6)cc5c4)CC3)cc2)C(=O)N1 ; O=C1CCC(c2ccc(C3CCN(CC(=O)Nc4ccc5c(F)c(N6CC(=O)NS6(=O)=O)c(O)cc5c4)CC3)cc2)C(=O)N1]

### Step 1: N-[7-benzyloxy-5-fluoro-6-(1,1,4-trioxo-1,2,5-thiadiazolidin-2-yl)-2-naphthyl]-2-[4-[4-(2,6-dioxo-3-piperidyl)phenyl]-1-piperidyl]acetamide (3)

Into a 8 mL screw-capped vial containing a well-stirred solution of 2-[4-[4-(2,6-dioxo-3-piperidyl)phenyl]-1-piperidyl]acetic acid (1, 85 mg, 227.08 μmol, HCl salt) in DMF at room temperature were added DIPEA (146.74 mg, 1.14 mmol, 197.76 μL) and propanephosphonic anhydride (50 Wt % in EtOAc, 289.00 mg, 454.15 μmol) and the resulting mixture was stirred at room temperature for 1 h. Subsequently, 5-(6-amino-3-benzyloxy-1-fluoro-2-naphthyl)-1,1-dioxo-1,2,5-thiadiazolidin-3-one (2, 120.66 mg, 227.08 μmol, TFA salt) was added and the resulting mixture was stirred at room temperature for 5 h. The solvent was evaporated and water was added to precipitate a solid that was filtered and dried to afford N-[7-benzyloxy-5-fluoro-6-(1,1,4-trioxo-1,2,5-thiadiazolidin-2-yl)-2-naphthyl]-2-[4-[4-(2,6-dioxo-3-piperidyl)phenyl]-1-piperidyl]acetamide (3, 90 mg, 118.53 μmol, 52% yield) as an off-white solid.

UPLC-MS (ES−): m/z 712.7 [M−H]⁻

### Step 2: 2-[4-[4-(2,6-dioxo-3-piperidyl)phenyl]-1-piperidyl]-N-[5-fluoro-7-hydroxy-6-(1,1,4-trioxo-1,2,5-thiadiazolidin-2-yl)-2-naphthyl]acetamide (Degrader 128)

Into a 25 mL round-bottom flask containing a well-stirred solution of N-[7-benzyloxy-5-fluoro-6-(1,1,4-trioxo-1,2,5-thiadiazolidin-2-yl)-2-naphthyl]-2-[4-[4-(2,6-dioxo-3-piperidyl)phenyl]-1-piperidyl]acetamide (3, 80 mg, 105.36 μmol) in anhydrous toluene (2 mL) and DCM (2 mL) under inert atmosphere was added pentamethylbenzene (78.09 mg, 526.78 μmol) and the resulting mixture was cooled to −78° C. Then Boron trichloride (1.0 M in DCM, 3.16 mmol, 0.3 mL) was added dropwise. Subsequently, the reaction mixture was brought to room temperature and stirred for 6 h. The reaction mixture was cooled to −78° C. and quenched slowly with 5% dichloromethane in methanol (2 mL). The reaction mixture was allowed to attain room temperature and concentrated under reduced pressure to get the crude material that was purified by reverse-phase preparative HPLC [column: X-SELECT C18 (250×19) MM, 5 μm; Mobile Phase A: 0.1% TFA in water and Mobile Phase B: acetonitrile] to afford 2-[4-[4-(2,6-dioxo-3-piperidyl)phenyl]-1-piperidyl]-N-[5-fluoro-7-hydroxy-6-(1,1,4-trioxo-1,2,5-thiadiazolidin-2-yl)-2-naphthyl]acetamide (Degrader 128, 13 mg, 17.24 μmol, 16% yield, TFA salt) as a white solid.

LCMS (ES−): 622.0 [M−H]⁻

¹H-NMR (400 MHz, DMSO-d6): δ 10.89 (s, 1H), 10.78 (brs, 1H), 9.82 (s, 1H), 8.12 (s, 1H), 7.91 (d, J=8.80 Hz, 1H), 7.48 (d, J=10.40 Hz, 1H), 7.38-7.12 (m, 4H), 7.01 (s, 1H), 4.23 (brs, 1H), 4.10 (s, 2H), 3.85 (dd, J=5.20, 11.40 Hz, 1H), 3.75-3.60 (m, 2H), 2.91-2.77 (m, 1H), 2.72-2.62 (m, 2H), 2.25-2.12 (m, 2H), 2.11-1.95 (m, 6H).

[Note: 2 protons merged in the solvent peak]

### 1-[2-[1-[3-(2,4-dioxohexahydropyrimidin-1-yl)-5-fluoro-1-methyl-indazol-6-yl]-4-piperidyl]ethyl]-3-[5-fluoro-7-hydroxy-6-(1,1,4-trioxo-1,2,5-thiadiazolidin-2-yl)-2-naphthyl]urea (Degrader 129)

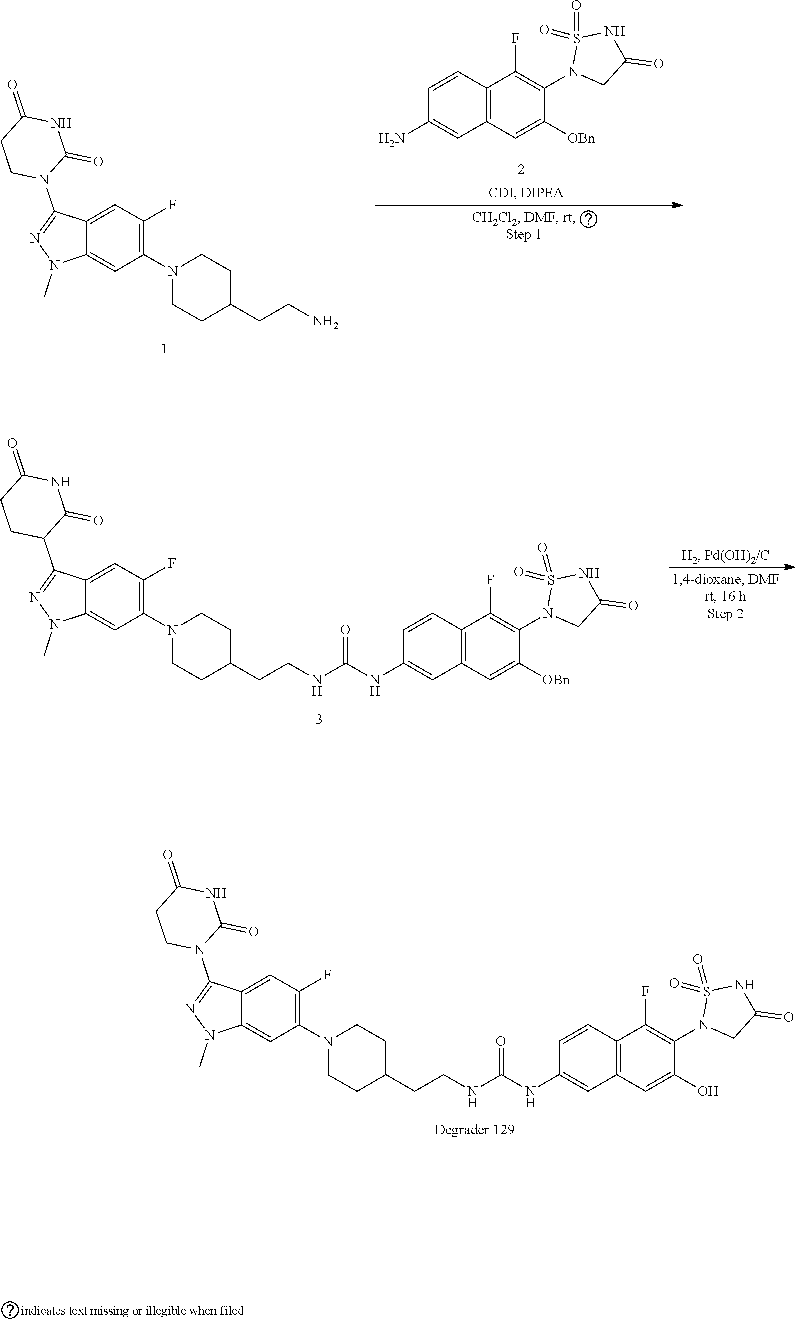 [SMILES: Cn1nc(N2CCC(=O)NC2=O)c2cc(F)c(N3CCC(CCN)CC3)cc21 ; Nc1ccc2c(F)c(N3CC(=O)NS3(=O)=O)c(OCc3ccccc3)cc2c1 ; Cn1nc(C2CCC(=O)NC2=O)c2cc(F)c(N3CCC(CCNC(=O)Nc4ccc5c(F)c(N6CC(=O)NS6(=O)=O)c(OCc6ccccc6)cc5c4)CC3)cc21 ; "H2, Pd(OH)2/C" ; Cn1nc(N2CCC(=O)NC2=O)c2cc(F)c(N3CCC(CCNC(=O)Nc4ccc5c(F)c(N6CC(=O)NS6(=O)=O)c(O)cc5c4)CC3)cc21]

### Step 1: 1-[7-benzyloxy-5-fluoro-6-(1,1,4-trioxo-1,2,5-thiadiazolidin-2-yl)-2-naphthyl]-3-[2-[1-[3-(2,4-dioxohexahydropyrimidin-1-yl)-5-fluoro-1-methyl-indazol-6-yl]-4-piperidyl]ethyl]urea (3)

Into a 10 mL single-neck round-bottom flask containing a well-stirred solution of 5-(6-amino-3-benzyloxy-1-fluoro-2-naphthyl)-1,1-dioxo-1,2,5-thiadiazolidin-3-one (2, 67.31 mg, 116.22 μmol, TFA salt) in anhydrous DCM (1.5 mL) and DMF (1.5 mL) were added 1,1′-carbonyldiimidazole (56.54 mg, 348.67 μmol) and DIPEA (89.04 mg, 688.93 μmol, 120.00 μL) at room temperature. The resulting mixture was stirred at room temperature for 2 h. Subsequently, 1-[6-[4-(2-aminoethyl)-1-piperidyl]-5-fluoro-1-methyl-indazol-3-yl]hexahydropyrimidine-2,4-dione (1, 60 mg, 116.22 μmol, TFA salt) and DIPEA (89.04 mg, 688.93 μmol, 120.00 μL) in anhydrous DMF (1.5 mL) were added and stirring was continued for another 16 h at room temperature. The reaction mixture was concentrated under reduced pressure and acidified with 1.5 N HCl (30 mL). The precipitates solid was filtered and dried under reduced pressure to get the 1-[7-benzyloxy-5-fluoro-6-(1,1,4-trioxo-1,2,5-thiadiazolidin-2-yl)-2-naphthyl]-3-[2-[1-[3-(2,4-dioxohexahydropyrimidin-1-yl)-5-fluoro-1-methyl-indazol-6-yl]-4-piperidyl]ethyl]urea (3, 90 mg, 90.81 μmol, 78% yield, HCl salt) as off white solid.

UPLC-MS (ES+): m/z 816.8 [M+H]⁺

### Step 2: 1-[2-[1-[3-(2,4-dioxohexahydropyrimidin-1-yl)-5-fluoro-1-methyl-indazol-6-yl]-4-piperidyl]ethyl]-3-[5-fluoro-7-hydroxy-6-(1,1,4-trioxo-1,2,5-thiadiazolidin-2-yl)-2-naphthyl]urea (Degrader 129)

Into a 10 mL single-neck round-bottom flask containing well-stirred solution of 1-[7-benzyloxy-5-fluoro-6-(1,1,4-trioxo-1,2,5-thiadiazolidin-2-yl)-2-naphthyl]-3-[2-[1-[3-(2,4-dioxohexahydropyrimidin-1-yl)-5-fluoro-1-methyl-indazol-6-yl]-4-piperidyl]ethyl]urea (3, 80 mg, 80.72 μmol, HCl salt) in anhydrous 1,4-dioxane (1.5 mL) and DMF (1.5 mL) was added palladium hydroxide on carbon (20 wt. % 50% water, 80 mg, 113.93 μmol) at room temperature. The resulting mixture was stirred at room temperature for 16 h under a hydrogen gas bladder. The reaction mixture was passed through Celite and concentrated under reduced pressure to get the crude material that was purified by reverse-phase preparative HPLC [Column: X-Select C18 (150×19) mm, 5 μm; Mobile phase A: 0.1% TFA in water and Mobile Phase B: MeCN] to get the 1-[2-[1-[3-(2,4-dioxohexahydropyrimidin-1-yl)-5-fluoro-1-methyl-indazol-6-yl]-4-piperidyl]ethyl]-3-[5-fluoro-7-hydroxy-6-(1,1,4-trioxo-1,2,5-thiadiazolidin-2-yl)-2-naphthyl]urea (Degrader 129, 25 mg, 29.48 μmol, 37% yield, TFA salt) as an off-white solid.

¹H-NMR (400 MHz, DMSO-d6): δ 10.54 (s, 1H), 10.47 (brs, 1H), 8.76 (s, 1H), 7.94 (s, 1H), 7.79 (d, J=8.80 Hz, 1H), 7.34 (d, J=12.40 Hz, 1H), 7.29 (dd, J=2.00, 9.20 Hz, 1H), 7.10 (d, J=7.20 Hz, 1H), 6.91 (s, 1H), 6.31 (s, 1H), 4.46 (s, 2H), 3.94 (s, 3H), 3.89 (t, J=6.80 Hz, 2H), 3.15-3.18 (m, 2H), 2.74 (t, J=6.80 Hz, 2H), 2.69-2.67 (m, 1H), 1.86-1.84 (m, 2H), 1.50-1.38 (m, 5H).

[Note: 3 aliphatic protons are eclipsed by the solvent peak]

LCMS (ES+): m/z 726.0 [M+H]⁺

### 2-[(3R,4R)-4-[1-(2,6-dioxo-3-piperidyl)-3-ethyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Degrader 130)

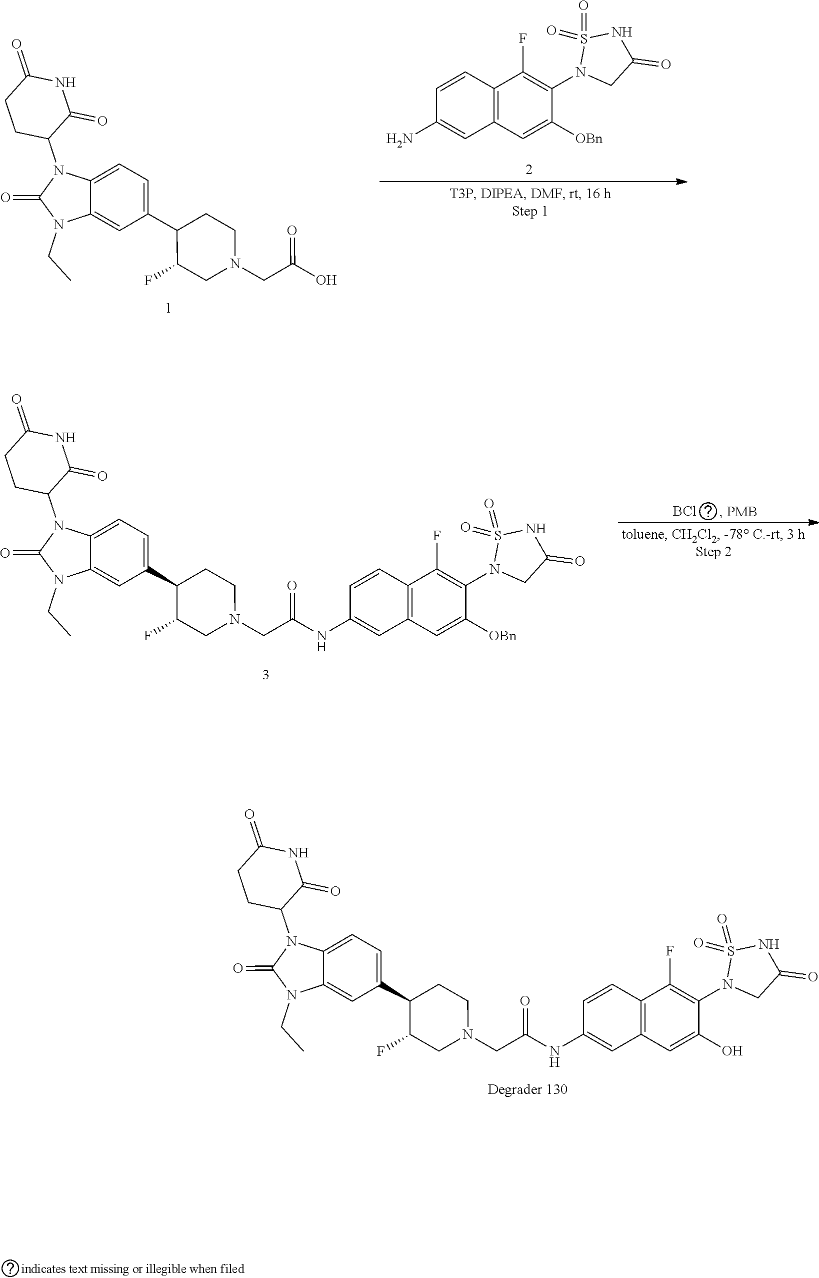 [SMILES: CCn1c(=O)n(C2CCC(=O)NC2=O)c2ccc(C3CCN(CC(=O)O)C[C@@H]3F)cc21 ; Nc1ccc2c(F)c(N3CC(=O)NS3(=O)=O)c(OCc3ccccc3)cc2c1 ; CCn1c(=O)n(C2CCC(=O)NC2=O)c2ccc([C@H]3CCN(CC(=O)Nc4ccc5c(F)c(N6CC(=O)NS6(=O)=O)c(OCc6ccccc6)cc5c4)C[C@@H]3F)cc21 ; "BCl" ; CCn1c(=O)n(C2CCC(=O)NC2=O)c2ccc([C@H]3CCN(CC(=O)Nc4ccc5c(F)c(N6CC(=O)NS6(=O)=O)c(O)cc5c4)C[C@@H]3F)cc21]

Note: Configurations are arbitrarily assigned.

### Step 1: N-[7-benzyloxy-5-fluoro-6-(1,1,4-trioxo-1,2,5-thiadiazolidin-2-yl)-2-naphthyl]-2-[(3R,4R)-4-[1-(2,6-dioxo-3-piperidyl)-3-ethyl-2-oxo-benzimidazol-5-yl]-3-fluoro-1-piperidyl]acetamide (3)

Into a 50 mL single-neck round-bottom flask containing a well-stirred solution of 2-[(3R,4R)-4-[1-(2,6-dioxo-3-piperidyl)-3-ethyl-2-oxo-benzimidazol-5-yl]-3-fluoro-1-piperidyl]acetic acid (1, 160 mg, 283.21 μmol, HCl salt) in anhydrous DMF (0.5 mL) were added DIPEA (366.02 mg, 2.83 mmol, 493.29 μL) and propylphosphonic anhydride solution (50 Wt % in EtOAc, 360.45 mg, 566.43 μmol) and stirred for 1 h at room temperature. Subsequently, 5-(6-amino-3-benzyloxy-1-fluoro-2-naphthyl)-1,1-dioxo-1,2,5-thiadiazolidin-3-one (2, 8.47 mg, 15.93 μmol, TFA salt) was added and stirring was continued for another 16 h. The reaction mixture was concentrated and the resulting crude material was purified by reverse phase column chromatography [Column: Silicycle-C18 120 g; Mobile Phase A: 0.1% Formic acid in water and Mobile Phase B: MeCN] to afford N-[7-benzyloxy-5-fluoro-6-(1,1,4-trioxo-1,2,5-thiadiazolidin-2-yl)-2-naphthyl]-2-[(3R,4R)-4-[1-(2,6-dioxo-3-piperidyl)-3-ethyl-2-oxo-benzimidazol-5-yl]-3-fluoro-1-piperidyl]acetamide (3, 90 mg, 93.04 μmol, 33% yield, formic acid salt) as an off-white solid.

LCMS (ES+): m/z 816.9 [M+H]⁺

### Step 2: 2-[(3R,4R)-4-[1-(2,6-dioxo-3-piperidyl)-3-ethyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Degrader 130)

Into a 50 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[(3R,4R)-4-[1-(2,6-dioxo-3-piperidyl)-3-ethyl-2-oxo-benzimidazol-5-yl]-3-fluoro-1-piperidyl]acetamide (3, 80 mg, 82.61 μmol, formic acid salt) and pentamethylbenzene (61.23 mg, 413.06 μmol) in anhydrous toluene (2 mL) and DCM (2 mL) under nitrogen atmosphere was added BCl₃ (1.0 M solution in DCM, 1.65 mmol, 1.65 mL) at −78° C. The resultant reaction mixture was stirred at room temperature for 3 h and then quenched with 1 mL of 5% MeOH in DCM at −78° C. Subsequently, the reaction mixture was concentrated under reduced pressure and the resulting crude material was purified by reverse-phase preparative HPLC [Column: X-Select C18 (150×30) mm, 5 μm; Mobile phase A: 0.1% Formic acid in water and Mobile Phase B: MeCN] to afford 2-[(3R,4R)-4-[1-(2,6-dioxo-3-piperidyl)-3-ethyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Degrader 130, 35 mg, 44.59 μmol, 54% yield, formic acid salt) as an off-white solid.

¹H-NMR (400 MHz, DMSO-d6): δ 11.15 (s, 1H), 10.75 (brs, 1H), 10.32 (brs, 1H), 10.03 (s, 1H), 8.11 (s, 1H), 7.90 (d, J=9.20 Hz, 1H), 7.45 (d, J=9.20 Hz, 1H), 7.29 (s, 1H), 7.15 (d, J=8.00 Hz, 1H), 7.03 (d, J=6.80 Hz, 1H), 7.00 (s, 1H), 5.38 (dd, J=5.60, 14.20 Hz, 1H), 5.20-4.95 (m, 1H), 4.35-4.10 (m, 4H), 4.00-3.88 (m, 2H), 3.80-3.70 (m, 2H), 3.00-2.81 (m, 2H), 2.80-2.60 (m, 3H), 2.30-2.10 (m, 1H), 2.19-1.97 (m, 1H), 1.26 (t, J=7.20 Hz, 3H),

LCMS (ES+): m/z 726.1 [M+H]⁺

[Note: 2 protons are eclipsed by the moisture signal in the solvent]

### 2-[(3S,4S)-4-[1-(2,6-dioxo-3-piperidyl)-3-ethyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Degrader 131)

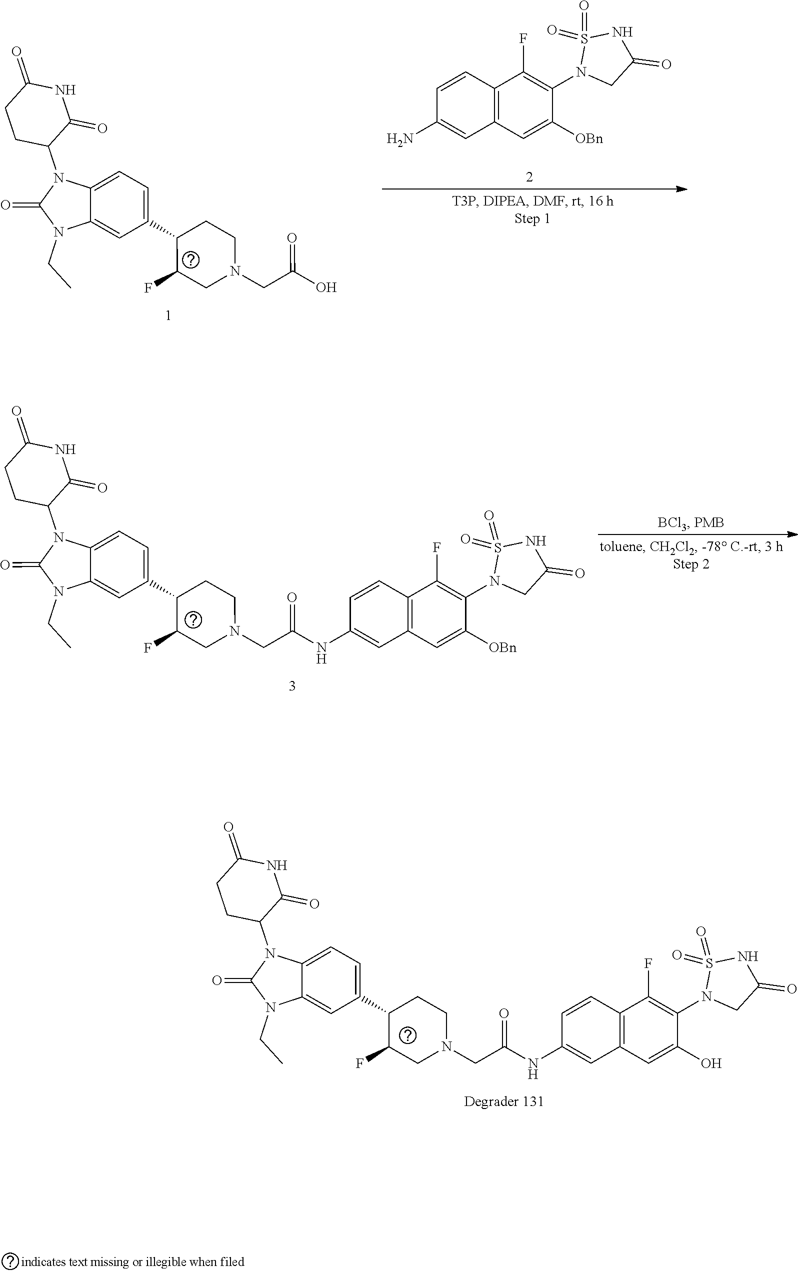

Note: Configurations are arbitrarily assigned.

### Step 1: N-[7-benzyloxy-5-fluoro-6-(1,1,4-trioxo-1,2,5-thiadiazolidin-2-yl)-2-naphthyl]-2-[(3S,4S)-4-[1-(2,6-dioxo-3-piperidyl)-3-ethyl-2-oxo-benzimidazol-5-yl]-3-fluoro-1-piperidyl]acetamide (3)

Into a 50 mL single-neck round-bottom flask containing a well-stirred solution of 2-[(3S,4S)-4-[1-(2,6-dioxo-3-piperidyl)-3-ethyl-2-oxo-benzimidazol-5-yl]-3-fluoro-1-piperidyl]acetic acid (1, 200 mg, 388.14 μmol, HCl salt) in anhydrous DMF (0.5 mL) were added DIPEA (501.63 mg, 3.88 mmol, 676.05 μL) and propylphosphonic anhydride solution (50 Wt % in EtOAc, 493.99 mg, 776.28 μmol) and stirred for 1 h at room temperature. Subsequently, 5-(6-amino-3-benzyloxy-1-fluoro-2-naphthyl)-1,1-dioxo-1,2,5-thiadiazolidin-3-one (2, 202.08 mg, 388.14 μmol, TFA salt) was added and stirring was continued for another 16 h. The reaction mixture was concentrated and the resulting crude material was purified by reverse phase column chromatography [Column: Silicycle-C18 120 g; Mobile Phase A: 0.1% Formic acid in water and Mobile Phase B: MeCN] to afford N-[7-benzyloxy-5-fluoro-6-(1,1,4-trioxo-1,2,5-thiadiazolidin-2-yl)-2-naphthyl]-2-[(3S,4S)-4-[1-(2,6-dioxo-3-piperidyl)-3-ethyl-2-oxo-benzimidazol-5-yl]-3-fluoro-1-piperidyl]acetamide (3, 140 mg, 158.73 μmol, 41% yield, formic acid salt) as an off-white solid.

LCMS (ES+): m/z 816.9 [M+H]⁺

### Step 2: 2-[(3S,4S)-4-[1-(2,6-dioxo-3-piperidyl)-3-ethyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Degrader 131)

Into a 50 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[(3S,4S)-4-[1-(2,6-dioxo-3-piperidyl)-3-ethyl-2-oxo-benzimidazol-5-yl]-3-fluoro-1-piperidyl]acetamide (3, (140 mg, 149.44 μmol, formic acid salt) and pentamethylbenzene (110.77 mg, 747.22 μmol) in anhydrous toluene (2 mL) and DCM (2 mL) under nitrogen atmosphere was added BCl₃ (1.0 M solution in DCM, 2.99 mmol, 3 mL) at −78° C. The resultant reaction mixture was stirred at room temperature for 2 h and then quenched with 5 mL of 5% MeOH in DCM at −78° C. Subsequently, the reaction mixture was concentrated under reduced pressure and the resulting crude material was purified by reverse-phase preparative HPLC [Column: X-Select C18 (150×30) mm, 5 μm; Mobile phase A: 0.1% Formic acid in water and Mobile Phase B: MeCN] to afford 2-[(3S,4S)-4-[1-(2,6-dioxo-3-piperidyl)-3-ethyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Degrader 131 55 mg, 70.52 μmol, 47% yield, formic acid salt) as an off-white solid.

¹H-NMR (400 MHz, DMSO-d6): δ 11.12 (s, 1H), 10.81 (brs, 1H), 10.36 (brs, 1H), 9.92 (s, 1H), 8.11 (s, 1H), 7.89 (d, J=8.80 Hz, 1H), 7.45 (d, J=8.40 Hz, 1H), 7.29 (s, 1H), 7.14 (d, J=7.60 Hz, 1H), 7.03 (d, J=7.60 Hz, 1H), 6.99 (s, 1H), 5.38 (dd, J=5.20, 12.80 Hz, 1H), 5.31-5.00 (m, 1H), 4.30-4.10 (m, 4H), 3.93-3.88 (m, 2H), 3.75-3.60 (m, 2H), 3.00-2.80 (m, 1H), 2.80-2.60 (m, 3H), 2.35-2.10 (m, 1H), 2.09-1.95 (m, 1H), 1.26 (t, J=7.20 Hz, 3H).

[Note: 2 aliphatic protons are eclipsed by the moisture signal in the solvent]

LCMS (ES+): m/z 726.1 [M+H]⁺

### 2-[(3S,4S)-1-[1-(2,6-dioxo-3-piperidyl)-3-methyl-2-oxo-benzimidazol-5-yl]-3-methyl-4-piperidyl]-N-[5-fluoro-7-hydroxy-6-(1,1,4-trioxo-1,2,5-thiadiazolidin-2-yl)-2-naphthyl]acetamide (Degrader 132)

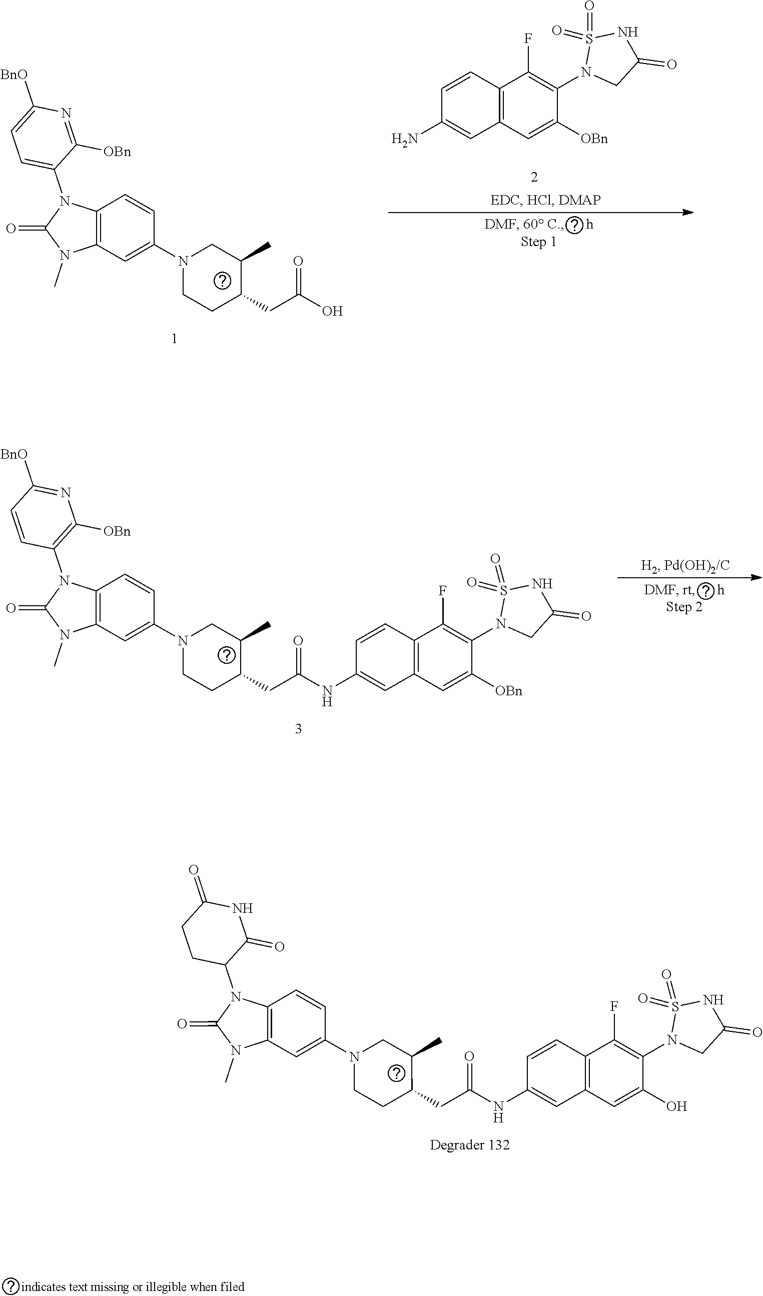 [SMILES: C[C@@H]1CN(c2ccc3c(c2)n(C)c(=O)n3-c2ccc(OCc3ccccc3)nc2OCc2ccccc2)CC[C@H]1CC(=O)O ; Nc1ccc2c(F)c(N3CC(=O)NS3(=O)=O)c(OCc3ccccc3)cc2c1 ; C[C@@H]1CN(c2ccc3c(c2)n(C)c(=O)n3-c2ccc(OCc3ccccc3)nc2OCc2ccccc2)CC[C@H]1CC(=O)Nc1ccc2c(F)c(N3CC(=O)NS3(=O)=O)c(OCc3ccccc3)cc2c1 ; "H2, Pd(OH)2/C" ; C[C@@H]1CN(c2ccc3c(c2)n(C)c(=O)n3C2CCC(=O)NC2=O)CC[C@H]1CC(=O)Nc1ccc2c(F)c(N3CC(=O)NS3(=O)=O)c(O)cc2c1]

Note: Configurations are arbitrarily assigned.

### Step 1: N-[7-benzyloxy-5-fluoro-6-(1,1,4-trioxo-1,2,5-thiadiazolidin-2-yl)-2-naphthyl]-2-[(3S,4S)-1-[1-(2,6-dibenzyloxy-3-pyridyl)-3-methyl-2-oxo-benzimidazol-5-yl]-3-methyl-4-piperidyl]acetamide (3)

Into a 20 mL screw-capped vial containing a well-stirred solution of 2-[(3S,4S)-1-[1-(2,6-dibenzyloxy-3-pyridyl)-3-methyl-2-oxo-benzimidazol-5-yl]-3-methyl-4-piperidyl]acetic acid (1, 80 mg, 122.75 μmol) in DMF (0.6 mL) at room temperature were added DMAP (89.98 mg, 736.50 μmol) and EDC·HCl (70.59 mg, 368.25 μmol). The resulting mixture was stirred at room temperature for 1 h. Subsequently 5-(6-amino-3-benzyloxy-1-fluoro-2-naphthyl)-1,1-dioxo-1,2,5-thiadiazolidin-3-one (2, 71.09 mg, 122.75 μmol, TFA salt) was added and the mixture was heated at 60° C. for 16 h. The reaction mixture treated with ice-cold water (10 mL) to precipitate a solid that was filtered, dried and purified by reverse-phase column chromatography [C18-50 g; Mobile Phase A: 0.1% Ammonium bicarbonate in water and Mobile Phase B: acetonitrile] to obtain N-[7-benzyloxy-5-fluoro-6-(1,1,4-trioxo-1,2,5-thiadiazolidin-2-yl)-2-naphthyl]-2-[(3S,4S)-1-[1-(2,6-dibenzyloxy-3-pyridyl)-3-methyl-2-oxo-benzimidazol-5-yl]-3-methyl-4-piperidyl]acetamide (3, 50 mg, 47.13 μmol, 38% yield) as an off-white solid.

UPLC-MS (ES+): m/z 976.4 [M+H]⁺

### Step 2: 2-[(3S,4S)-1-[1-(2,6-dioxo-3-piperidyl)-3-methyl-2-oxo-benzimidazol-5-yl]-3-methyl-4-piperidyl]-N-[5-fluoro-7-hydroxy-6-(1,1,4-trioxo-1,2,5-thiadiazolidin-2-yl)-2-naphthyl]acetamide (Degrader 132)

Into a 25 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[(3S,4S)-1-[1-(2,6-dibenzyloxy-3-pyridyl)-3-methyl-2-oxo-benzimidazol-5-yl]-3-methyl-4-piperidyl]acetamide (3, 150 mg, 141.38 μmol) in anhydrous DMF (1 mL) and 1,4-dioxane (3 mL) was added palladium hydroxide on carbon (99.27 mg, 141.38 μmol, 20% purity) at room temperature. The resulting mixture was stirred at room temperature for 16 h under a hydrogen gas bladder. The reaction mixture was passed through Celite and concentrated under reduced pressure to get the crude compound that was purified by reverse-phase preparative HPLC [Column: X-Select C8 (150×19) mm, 5 μm; Mobile Phase A: 0.1% TFA in water and Mobile Phase B: MeCN] to get the 2-[(3S,4S)-1-[1-(2,6-dioxo-3-piperidyl)-3-methyl-2-oxo-benzimidazol-5-yl]-3-methyl-4-piperidyl]-N-[5-fluoro-7-hydroxy-6-(1,1,4-trioxo-1,2,5-thiadiazolidin-2-yl)-2-naphthyl]acetamide (Degrader 132, 22 mg, 26.43 μmol, 19% yield, TFA salt) as an off-white solid.

¹H-NMR (400 MHz, DMSO-d6): δ 11.12 (s, 1H), 10.22 (s, 1H), 10.03 (brs, 1H), 8.18 (s, 1H), 7.86 (d, J=9.20 Hz, 1H), 7.46 (dd, J=1.60, 8.80 Hz, 1H), 7.40-7.00 (m, 3H), 6.96 (s, 1H), 5.49-5.35 (m, 1H), 4.23 (s, 2H), 3.36 (s, 3H), 3.02-2.87 (m, 2H), 2.75-2.65 (m, 2H), 2.30-2.18 (m, 1H), 2.08-1.63 (m, 5H), 1.02 (d, J=4.80 Hz, 3H).

[Note: 4 aliphatic protons are eclipsed by the solvent signal]

LCMS (ES+): m/z 708.2 [M+H]⁺

### 2-[(3R,4R)-1-[1-(2,6-dioxo-3-piperidyl)-3-methyl-2-oxo-benzimidazol-5-yl]-3-methyl-4-piperidyl]-N-[5-fluoro-7-hydroxy-6-(1,1,4-trioxo-1,2,5-thiadiazolidin-2-yl)-2-naphthyl]acetamide (Degrader 133)

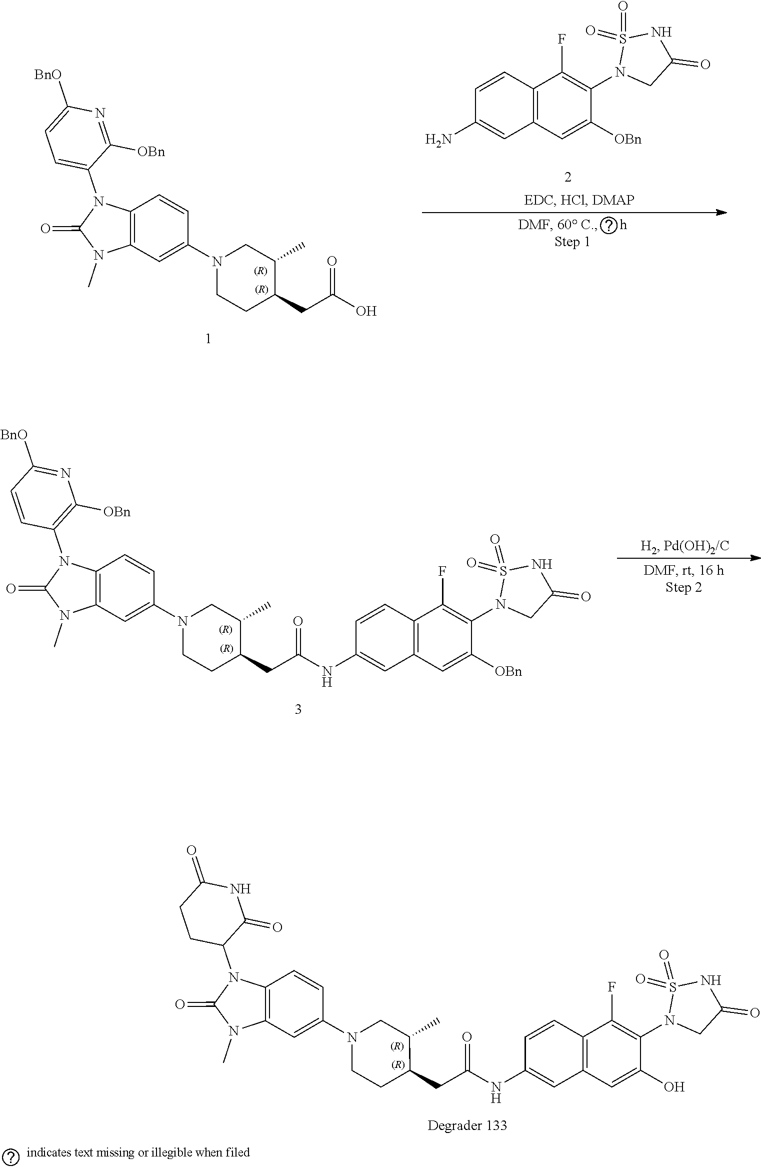

Note: Configurations are arbitrarily assigned.

### Step 1: N-[7-benzyloxy-5-fluoro-6-(1,1,4-trioxo-1,2,5-thiadiazolidin-2-yl)-2-naphthyl]-2-[(3R,4R)-1-[1-(2,6-dibenzyloxy-3-pyridyl)-3-methyl-2-oxo-benzimidazol-5-yl]-3-methyl-4-piperidyl]acetamide (3)

Into a 20 mL screw-capped vial containing a well-stirred solution of 2-[(3R,4R)-1-[1-(2,6-dibenzyloxy-3-pyridyl)-3-methyl-2-oxo-benzimidazol-5-yl]-3-methyl-4-piperidyl]acetic acid (1, 80 mg, 133.63 μmol) in DMF (4 mL) at room temperature were added DMAP (97.95 mg, 801.78 μmol) and EDC·HCl (76.85 mg, 400.89 μmol). The resulting mixture was stirred at room temperature for 1 h. Subsequently 5-(6-amino-3-benzyloxy-1-fluoro-2-naphthyl)-1,1-dioxo-1,2,5-thiadiazolidin-3-one (2, 70.85 mg, 133.63 μmol, TFA salt) was added and the mixture was heated at 60° C. for 16 h. The reaction mixture treated with ice-cold water (10 mL) to precipitate a solid that was filtered, dried and purified by reverse-phase column chromatography [C18-50 g; Mobile Phase A: 0.1% Ammonium bicarbonate in water and Mobile Phase B: acetonitrile] to obtain N-[7-benzyloxy-5-fluoro-6-(1,1,4-trioxo-1,2,5-thiadiazolidin-2-yl)-2-naphthyl]-2-[(3R,4R)-1-[1-(2,6-dibenzyloxy-3-pyridyl)-3-methyl-2-oxo-benzimidazol-5-yl]-3-methyl-4-piperidyl]acetamide (3, 150 mg, 132.16 μmol, 99% yield) as an off-white solid.

UPLC-MS (ES+): m/z 976.4 [M+H]⁺

### Step 2: 2-[(3R,4R)-1-[1-(2,6-dioxo-3-piperidyl)-3-methyl-2-oxo-benzimidazol-5-yl]-3-methyl-4-piperidyl]-N-[5-fluoro-7-hydroxy-6-(1,1,4-trioxo-1,2,5-thiadiazolidin-2-yl)-2-naphthyl]acetamide (Degrader 133)

Into a 25 mL single-neck round-bottom flask containing well-stirred solution of N-[7-benzyloxy-5-fluoro-6-(1,1,4-trioxo-1,2,5-thiadiazolidin-2-yl)-2-naphthyl]-2-[(3R,4R)-1-[1-(2,6-dibenzyloxy-3-pyridyl)-3-methyl-2-oxo-benzimidazol-5-yl]-3-methyl-4-piperidyl]acetamide (3, 150 mg, 132.16 μmol) in anhydrous DMF (1 mL) and 1,4-dioxane (3 mL) was added palladium hydroxide on carbon (92.80 mg, 132.16 μmol, 20% purity) at room temperature. The resulting mixture was stirred at room temperature for 16 h under a hydrogen gas bladder. The reaction mixture was passed through Celite and concentrated under reduced pressure to get the crude compound that was purified by reverse-phase preparative HPLC [Column: X-Select C8 (150×19) mm, 5 μm; Mobile Phase A: 0.1% TFA in water and Mobile Phase B: MeCN] to get the 2-[(3R,4R)-1-[1-(2,6-dioxo-3-piperidyl)-3-methyl-2-oxo-benzimidazol-5-yl]-3-methyl-4-piperidyl]-N-[5-fluoro-7-hydroxy-6-(1,1,4-trioxo-1,2,5-thiadiazolidin-2-yl)-2-naphthyl]acetamide (Degrader 133, 17 mg, 20.23 μmol, 15% yield, TFA salt) as an off white solid.

¹H-NMR (400 MHz, DMSO-d6): δ 11.12 (s, 1H), 10.23 (s, 1H), 10.15 (brs, 1H), 8.17 (s, 1H), 7.85 (d, J=8.80 Hz, 1H), 7.46 (dd, J=1.60, 9.20 Hz, 1H), 7.40-7.15 (m, 3H), 6.96 (s, 1H), 5.37 (dd, J=5.20, 12.60 Hz, 1H), 4.21 (s, 2H), 3.63-3.54 (m, 2H), 3.35 (s, 3H), 3.00-2.90 (m, 1H), 2.74-2.61 (m, 3H), 2.30-2.18 (m, 1H), 2.08-1.63 (m, 5H), 1.02 (d, J=4.80 Hz, 3H).

[Note: 2 aliphatic protons are eclipsed by the solvent signal]

LCMS (ES+): m/z 708.2 [M+H]⁺

### 2-[1-[3-(2,4-dioxohexahydropyrimidin-1-yl)-5-fluoro-1-methyl-indazol-6-yl]-4-piperidyl]-N-[5-fluoro-7-hydroxy-6-(1,1,4-trioxo-1,2,5-thiadiazolidin-2-yl)-2-naphthyl]acetamide (Degrader 134)

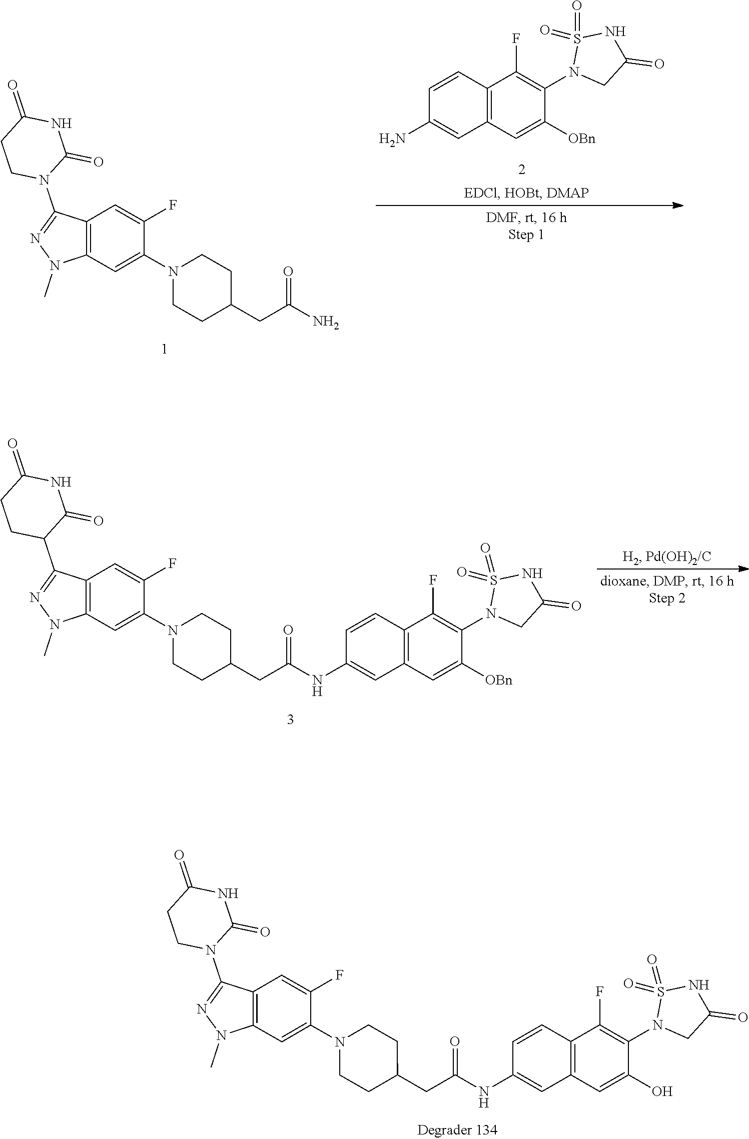 [SMILES: Cn1nc(N2CCC(=O)NC2=O)c2cc(F)c(N3CCC(CC(N)=O)CC3)cc21 ; Nc1ccc2c(F)c(N3CC(=O)NS3(=O)=O)c(OCc3ccccc3)cc2c1 ; Cn1nc(C2CCC(=O)NC2=O)c2cc(F)c(N3CCC(CC(=O)Nc4ccc5c(F)c(N6CC(=O)NS6(=O)=O)c(OCc6ccccc6)cc5c4)CC3)cc21 ; "H2, Pd(OH)2/C" ; Cn1nc(N2CCC(=O)NC2=O)c2cc(F)c(N3CCC(CC(=O)Nc4ccc5c(F)c(N6CC(=O)NS6(=O)=O)c(O)cc5c4)CC3)cc21]

### Step 1: N-[7-benzyloxy-5-fluoro-6-(1,1,4-trioxo-1,2,5-thiadiazolidin-2-yl)-2-naphthyl]-2-[1-[3-(2,4-dioxohexahydropyrimidin-1-yl)-5-fluoro-1-methyl-indazol-6-yl]-4-piperidyl]acetamide (3)

Into a 25 mL single-neck round-bottom flask containing a well-stirred solution of 2-[1-[3-(2,4-dioxohexahydropyrimidin-1-yl)-5-fluoro-1-methyl-indazol-6-yl]-4-piperidyl]acetic acid (1, 200 mg, 324.68 μmol, TFA salt) and 5-(6-amino-3-benzyloxy-1-fluoro-2-naphthyl)-1,1-dioxo-1,2,5-thiadiazolidin-3-one (2, 188.04 mg, 324.68 μmol, TFA salt) in anhydrous DMF (3 mL) were added DMAP (277.66 mg, 2.27 mmol), HOBt (131.61 mg, 974.05 μmol) and EDC·HCl (248.97 mg, 1.30 mmol) at room temperature. The reaction mixture was stirred at ambient temperature 16 h. The solvent was evaporated and the residue was treated with 1.5 N HCl (5 mL) at 0° C. The precipitated solid was collected by filtration and dried under vacuum to the obtain crude material that was purified by reverse-phase chromatography [Column: REDISEP C18-120 g; Mobile Phase A: 0.1 mM Ammonium bicarbonate in water and Mobile Phase B: MeCN] to get N-[7-benzyloxy-5-fluoro-6-(1,1,4-trioxo-1,2,5-thiadiazolidin-2-yl)-2-naphthyl]-2-[1-[3-(2,4-dioxohexahydropyrimidin-1-yl)-5-fluoro-1-methyl-indazol-6-yl]-4-piperidyl]acetamide (3, 170 mg, 172.85 μmol, 53% yield) as a light brown solid.

UPLC-MS (ES+): m/z 787.3 [M+H]⁺

### Step 2: 2-[1-[3-(2,4-dioxohexahydropyrimidin-1-yl)-5-fluoro-1-methyl-indazol-6-yl]-4-piperidyl]-N-[5-fluoro-7-hydroxy-6-(1,1,4-trioxo-1,2,5-thiadiazolidin-2-yl)-2-naphthyl]acetamide (Degrader 134)

Into a 25 mL single-neck round-bottom flask containing well-stirred solution of N-[7-benzyloxy-5-fluoro-6-(1,1,4-trioxo-1,2,5-thiadiazolidin-2-yl)-2-naphthyl]-2-[1-[3-(2,4-dioxohexahydropyrimidin-1-yl)-5-fluoro-1-methyl-indazol-6-yl]-4-piperidyl]acetamide (3, 150 mg, 152.52 μmol) in anhydrous DMF (1 mL) and 1,4-dioxane (3 mL) was added 20% Palladium hydroxide on carbon (107.09 mg, 152.52 μmol, 20% purity) at room temperature. The contents were stirred at room temperature for 16 h under hydrogen bladder pressure. The reaction was passed through Celite and washed with DMF. The filtrate was concentrated under reduced pressure to get the crude compound that was purified by reverse-phase preparative HPLC [Column: X-bridge C8 (150×19) mm, 5 μm; Mobile phase A: 0.1% TFA in water and Mobile Phase B: MeCN] to get 2-[1-[3-(2,4-dioxohexahydropyrimidin-1-yl)-5-fluoro-1-methyl-indazol-6-yl]-4-piperidyl]-N-[5-fluoro-7-hydroxy-6-(1,1,4-trioxo-1,2,5-thiadiazolidin-2-yl)-2-naphthyl]acetamide (Degrader 134, 25 mg, 30.59 μmol, 20% yield, TFA salt) as an off white solid.

¹H-NMR (400 MHz, DMSO-d6): δ 10.46 (s, 1H), 10.11 (s, 1H), 9.85 (s, 1H), 8.12 (s, 1H), 7.76 (d, J=9.20 Hz, 1H), 7.37 (dd, J=2.00, 9.20 Hz, 1H), 7.27 (d, J=12.80 Hz, 1H), 7.04 (d, J=7.20 Hz, 1H), 6.88 (s, 1H), 4.11 (s, 2H), 3.87 (s, 3H), 3.82 (t, J=6.40 Hz, 2H), 3.39-3.36 (m, 2H), 2.68-2.61 (m, 4H), 2.33 (d, J=7.20 Hz, 2H), 2.01-1.92 (m, 1H), 1.81-1.78 (m, 2H), 1.45-1.40 (m, 2H).

LCMS (ES+): m/z 697.2 [M+H]⁺

### 2-[[(3R)-1-[1-(2,6-dioxo-3-piperidyl)-3-methyl-2-oxo-benzimidazol-4-yl]-3-piperidyl]oxy]-N-[5-fluoro-7-hydroxy-6-(1,1,4-trioxo-1,2,5-thiadiazolidin-2-yl)-2-naphthyl]acetamide (Degrader 135)

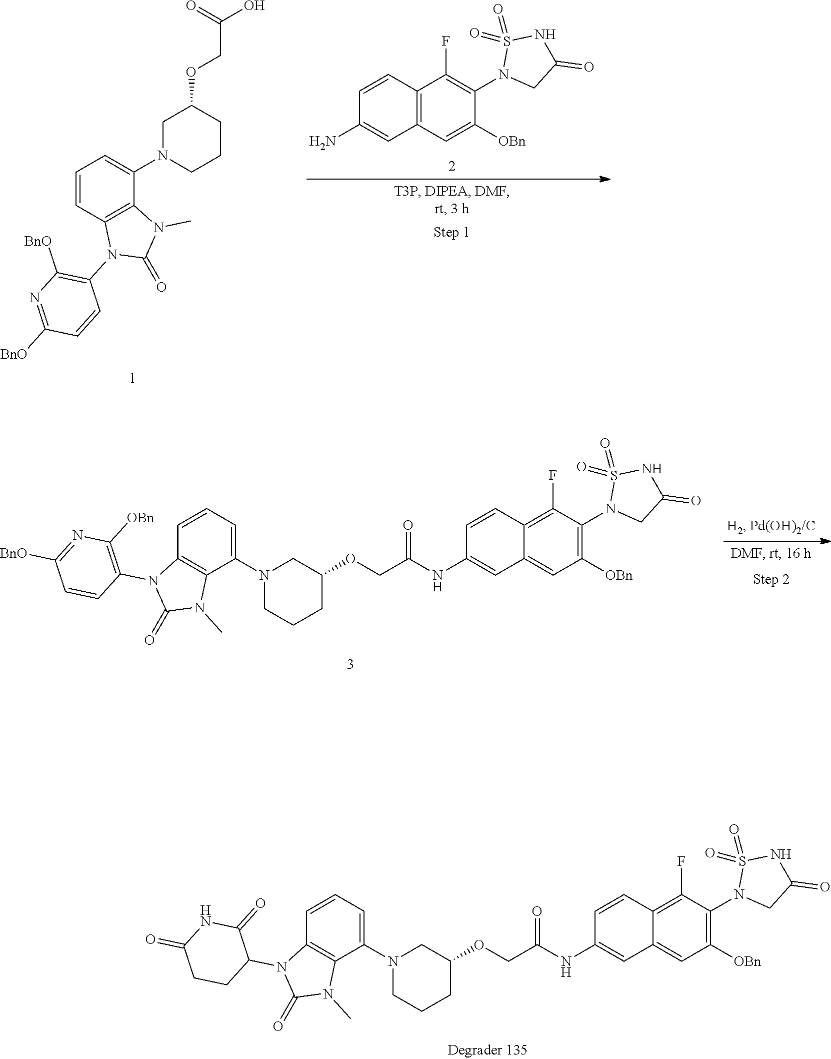 [SMILES: Cn1c(=O)n(-c2ccc(OCc3ccccc3)nc2OCc2ccccc2)c2cccc(N3CCC[C@@H](OCC(=O)O)C3)c21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2cccc(N3CCC[C@@H](OCC(=O)Nc4ccc5c(F)c(N6CC(=O)NS6(=O)=O)c(OCc6ccccc6)cc5c4)C3)c21 ; Cn1c(=O)n(C2CCC(=O)NC2=O)c2cccc(N3CCC[C@@H](OCC(=O)Nc4ccc5c(F)c(N6CC(=O)NS6(=O)=O)c(OCc6ccccc6)cc5c4)C3)c21]

Note: Configurations are arbitrarily assigned.

### Step 1: N-[7-benzyloxy-5-fluoro-6-(1,1,4-trioxo-1,2,5-thiadiazolidin-2-yl)-2-naphthyl]-2-[[(3R)-1-[1-(2,6-dibenzyloxy-3-pyridyl)-3-methyl-2-oxo-benzimidazol-4-yl]-3-piperidyl]oxy]acetamide (3)

Into a 10 mL single-neck round-bottom flask containing well-stirred solution of 2-[[(3R)-1-[1-(2,6-dibenzyloxy-3-pyridyl)-3-methyl-2-oxo-benzimidazol-4-yl]-3-piperidyl]oxy]acetic acid (1, 50 mg, 84.08 μmol) in anhydrous DMF (0.5 mL) were added propylphosphonic anhydride solution (>50 wt. % EtOAc, 321.04 mg, 504.49 μmol, 50% purity) and DIPEA (54.34 mg, 420.41 μmol, 73.23 μL) at room temperature. The resulting mixture was stirred at room temperature for 10 min and then 5-(6-amino-3-benzyloxy-1-fluoro-2-naphthyl)-1,1-dioxo-1,2,5-thiadiazolidin-3-one (2, 43.34 mg, 84.08 μmol, TFA salt) in anhydrous DMF (0.5 mL) was added. After stirring for another 3 h, the reaction mixture was concentrated under reduced pressure and treated with ice-cold water to get a precipitate that was filtered and dried to afford the N-[7-benzyloxy-5-fluoro-6-(1,1,4-trioxo-1,2,5-thiadiazolidin-2-yl)-2-naphthyl]-2-[[(3R)-1-[1-(2,6-dibenzyloxy-3-pyridyl)-3-methyl-2-oxo-benzimidazol-4-yl]-3-piperidyl]oxy]acetamide (3, 35 mg, 35.26 μmol, 42% yield) as an off-white solid.

UPLC-MS (ES+): m/z 978.2 [M+H]⁺

### Step 2: 2-[[(3R)-1-[1-(2,6-dioxo-3-piperidyl)-3-methyl-2-oxo-benzimidazol-4-yl]-3-piperidyl]oxy]-N-[5-fluoro-7-hydroxy-6-(1,1,4-trioxo-1,2,5-thiadiazolidin-2-yl)-2-naphthyl]acetamide (Degrader 135)

Into a 10 mL single-neck round-bottom flask containing well-stirred solution of N-[7-benzyloxy-5-fluoro-6-(1,1,4-trioxo-1,2,5-thiadiazolidin-2-yl)-2-naphthyl]-2-[[(3R)-1-[1-(2,6-dibenzyloxy-3-pyridyl)-3-methyl-2-oxo-benzimidazol-4-yl]-3-piperidyl]oxy]acetamide (3, 35 mg, 35.26 μmol) in anhydrous 1,4-dioxane (0.6 mL) and DMF (0.6 mL) was added palladium hydroxide on carbon (20 wt. % 50% water, 50 mg, 71.21 μmol) at room temperature. The resulting mixture was stirred at room temperature for 16 h under a hydrogen gas bladder. The reaction mixture was passed through Celite and washed with a 1:1 mixture of dioxane and DMF (50 mL). The filtrate was concentrated under reduced pressure to get the crude material that was purified by reverse-phase preparative-HPLC [Column: X-Bridge C8 (150×19) mm, 5 μm; Mobile phase A: 0.1% TFA in water and Mobile Phase B: MeCN] to afford 2-[[(3R)-1-[1-(2,6-dioxo-3-piperidyl)-3-methyl-2-oxo-benzimidazol-4-yl]-3-piperidyl]oxy]-N-[5-fluoro-7-hydroxy-6-(1,1,4-trioxo-1,2,5-thiadiazolidin-2-yl)-2-naphthyl]acetamide (Degrader 135, 5 mg, 6.04 μmol, 17% yield, TFA salt) as off white solid.

¹H NMR (400 MHz, DMSO-d6): δ 11.09 (s, 1H), 10.35 (brs, 1H), 9.88 (s, 1H), 8.15 (s, 1H), 7.86 (d, J=8.80 Hz, 1H), 7.55 (d, J=8.80 Hz, 1H), 7.02-6.89 (m, 4H), 5.36 (dd, J=5.20, 12.80 Hz, 1H), 4.52 (s, 2H), 4.36 (s, 2H), 3.82-3.61 (m, 5H), 3.12-3.01 (m, 1H), 2.92-2.85 (m, 2H), 2.74-2.60 (m, 4H), 2.33-2.25 (m, 1H), 2.09-1.99 (m, 1H), 1.79-1.50 (m, 2H), 1.33-1.18 (m, 1H).

LCMS (ES+): m/z 710.2 [M+H]⁺

### 2-[4-[[1-(2,6-dioxo-3-piperidyl)-3-methyl-2-oxo-benzimidazol-5-yl]methyl]phenyl]-N-[5-fluoro-7-hydroxy-6-(1,1,4-trioxo-1,2,5-thiadiazolidin-2-yl)-2-naphthyl]acetamide (Degrader 136)

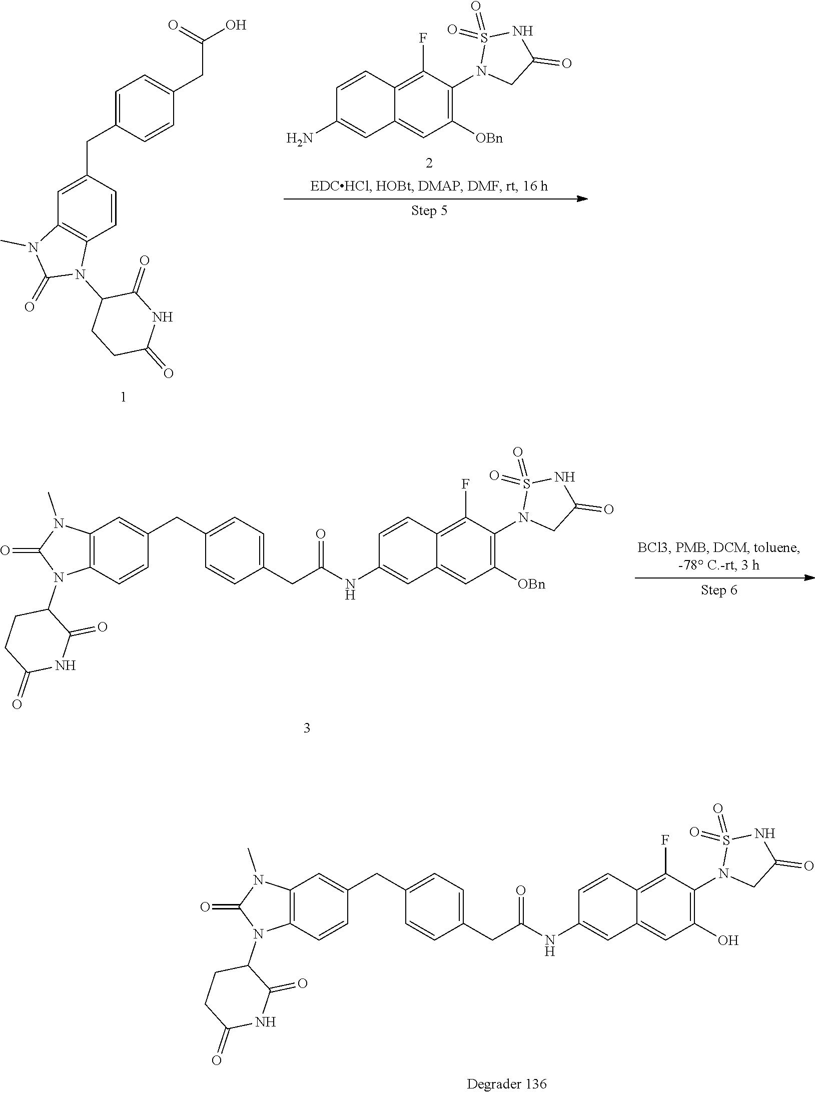 [SMILES: Cn1c(=O)n(C2CCC(=O)NC2=O)c2ccc(Cc3ccc(CC(=O)O)cc3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(Cc3ccc(CC(=O)Nc4ccc5c(F)c(N6CC(=O)NS6(=O)=O)c(OCc6ccccc6)cc5c4)cc3)cc21 ; Cn1c(=O)n(C2CCC(=O)NC2=O)c2ccc(Cc3ccc(CC(=O)Nc4ccc5c(F)c(N6CC(=O)NS6(=O)=O)c(O)cc5c4)cc3)cc21]

### Step 1: N-[7-benzyloxy-5-fluoro-6-(1,1,4-trioxo-1,2,5-thiadiazolidin-2-yl)-2-naphthyl]-2-[4-[[1-(2,6-dioxo-3-piperidyl)-3-methyl-2-oxo-benzimidazol-5-yl]methyl]phenyl]acetamide (3)

Into a 20 mL vial containing a well-stirred solution of 5-(6-amino-3-benzyloxy-1-fluoro-2-naphthyl)-1,1-dioxo-1,2,5-thiadiazolidin-3-one (2, 100 mg, 192.07 μmol, TFA salt) in anhydrous DMF (2.5 mL) were added 2-[4-[[1-(2,6-dioxo-3-piperidyl)-3-methyl-2-oxo-benzimidazol-5-yl]methyl]phenyl]acetic acid (1, 79.85 mg, 192.07 μmol), N-(3-dimethylaminopropyl)-N′-ethylcarbodiimide hydrochloride (110.46 mg, 576.21 μmol), HOBT (51.91 mg, 384.14 μmol) and DMAP (140.79 mg, 1.15 mmol). After 16 h, the solvent was removed under reduced pressure to get crude compound. The residue was treated with 1.5N HCl (3 mL) and The precipitate was filtered, washed with water and dried. The crude compound was purified by reverse phase column chromatography [silicycle C18 column; Mobile phase A: 0.1% formic acid in water; Mobile phase B: MeCN] to get N-[7-benzyloxy-5-fluoro-6-(1,1,4-trioxo-1,2,5-thiadiazolidin-2-yl)-2-naphthyl]-2-[4-[[1-(2,6-dioxo-3-piperidyl)-3-methyl-2-oxo-benzimidazol-5-yl]methyl]phenyl]acetamide (3, 50 mg, 61.44 μmol, 32% yield) as a colorless solid.

LCMS (ES+): m/z 791.0 [M+H]⁺

### Step 2: 2-[4-[[1-(2,6-dioxo-3-piperidyl)-3-methyl-2-oxo-benzimidazol-5-yl]methyl]phenyl]-N-[5-fluoro-7-hydroxy-6-(1,1,4-trioxo-1,2,5-thiadiazolidin-2-yl)-2-naphthyl]acetamide (Degrader 136)

Into a 50 mL single-neck round-bottom flask containing a well-stirred solution of N-[7-benzyloxy-5-fluoro-6-(1,1,4-trioxo-1,2,5-thiadiazolidin-2-yl)-2-naphthyl]-2-[4-[[1-(2,6-dioxo-3-piperidyl)-3-methyl-2-oxo-benzimidazol-5-yl]methyl]phenyl]acetamide (3, 50 mg, 61.33 μmol) in anhydrous toluene (1 mL) and DCM (1 mL) was added pentamethylbenzene (45.46 mg, 306.65 μmol) and the reaction mixture was cooled to −78° C. Then a 1.0 M solution of boron trichloride in DCM (1.23 mmol, 1.23 mL) was added dropwise. The reaction mixture was stirred at room temperature. After 3 h, the reaction mixture was cooled to −78° C. and quenched with 5% methanol in dichloromethane (0.5 mL). The reaction mixture was concentrated under reduced pressure at 30° C. and the residue was washed with diethyl ether (10 mL) and filtered. The crude compound was purified by reverse phase column chromatography [Silicycle C18 column, mobile phase A: 0.1% TFA in water; Mobile phase B: MeCN] to get 2-[4-[[1-(2,6-dioxo-3-piperidyl)-3-methyl-2-oxo-benzimidazol-5-yl]methyl]phenyl]-N-[5-fluoro-7-hydroxy-6-(1,1,4-trioxo-1,2,5-thiadiazolidin-2-yl)-2-naphthyl]acetamide (Degrader 136, 17 mg, 23.20 μmol, 38% yield) as an off-white solid

LCMS (ES−): m/z 699.0 [M−H]⁻

¹H NMR (400 MHz, DMSO-d6): δ 11.07 (s, 1H), 10.36 (s, 1H), 10.12 (bs, 1H), 8.13 (s, 1H), 7.83 (d, J=9.20 Hz, 1H), 7.43 (dd, J=2.00, 9.00 Hz, 1H), 7.27 (d, J=8.40 Hz, 2H), 7.21 (d, J=8.40 Hz, 2H), 7.09-7.08 (m, 1H), 7.01 (d, J=8.00 Hz, 1H), 6.96-6.90 (m, 2H), 5.35-5.30 (m, 1H), 4.24 (s, 2H), 3.95 (s, 2H), 3.64 (s, 2H), 3.30 (s, 3H), 2.90-2.84 (m, 1H), 2.70-2.63 (m, 3H), 2.00-1.97 (m, 1H).

### (2R)-2-[4-[1-(2,6-dioxo-3-piperidyl)-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]-3-hydroxy-propanamide (Degrader 137)

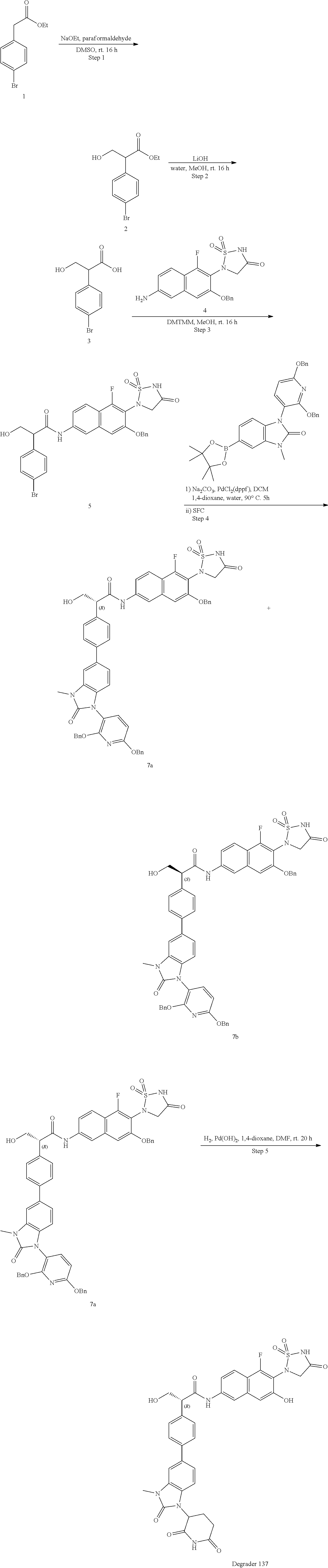

Note: Configurations are arbitrarily assigned.

### Step 1: ethyl 2-(4-bromophenyl)-3-hydroxy-propanoate (2)

Into a 250 mL single-neck round-bottom flask containing a well-stirred solution of ethyl 2-(4-bromophenyl)acetate (1, 10 g, 41.14 mmol) in anhydrous DMSO (50 mL) was added sodium ethoxide (167.96 mg, 2.47 mmol) at room temperature. After 5 min, paraformaldehyde (1.48 g, 49.36 mmol) was added and stirring was continued for 16 h at room temperature. The reaction mixture was quenched with acetic acid (0.8 mL) and water (100 mL). The aqueous layer was extracted with MTBE (3×70 mL). The combined organic layers were washed with 10% aqueous NaHCO₃ (1×40 mL), brine (1×40 mL), dried over anhydrous Na₂SO₄, filtered and concentrated under reduced pressure to get the crude material. Purification by a flash silica-gel (230-400 mesh) flash column chromatography (35% EtOAc in Pet-ether) afforded ethyl 2-(4-bromophenyl)-3-hydroxy-propanoate (2, 6.5 g, 22.61 mmol, 55% yield) as colorless oil.

### Step 2: 2-(4-bromophenyl)-3-hydroxy-propanoic acid (3)

Into a 50 mL single-neck round-bottom flask containing a well-stirred solution of ethyl 2-(4-bromophenyl)-3-hydroxy-propanoate (2, 1 g, 3.48 mmol) in anhydrous THF (8 mL) and methanol (8 mL) was added lithium hydroxide monohydrate (729.81 mg, 17.39 mmol) in water (8 mL) at room temperature. The contents were stirred at room temperature for 16 h. The reaction mixture was acidified with aqueous 1.5 N HCl solution and the aqueous layer was extracted with EtOAc (3×30 mL). The combined organic layers were dried over anhydrous Na₂SO₄, filtered and concentrated under reduced pressure to get 2-(4-bromophenyl)-3-hydroxy-propanoic acid (3, 0.7 g, 2.57 mmol, 74% yield) as white solid.

UPLC-MS (ES−): m/z 243.0 [M−H]⁻

### Step 3: N-[7-benzyloxy-5-fluoro-6-(1,1,4-trioxo-1,2,5-thiadiazolidin-2-yl)-2-naphthyl]-2-(4-bromophenyl)-3-hydroxy-propanamide (5)

Into a 50 mL single-neck round-bottom flask containing a well-stirred solution of 2-(4-bromophenyl)-3-hydroxy-propanoic acid (3, 400 mg, 1.47 mmol), 5-(6-amino-3-benzyloxy-1-fluoro-2-naphthyl)-1,1-dioxo-1,2,5-thiadiazolidin-3-one (4, 829.49 mg, 1.47 mmol, TFA salt) in anhydrous methanol (16 mL) was added 4-(4,6-Dimethoxy-1,3,5-triazin-2-yl)-4-methylmorpholinium chloride (609.74 mg, 2.20 mmol) at room temperature and the resultant mixture was stirred at room temperature for 16 h. The reaction mixture was concentrated and the residue was subjected to reverse-phase column chromatography [Silicycle C18 column; Mobile phase A: 0.1% FA in water and Mobile phase B: MeCN] to get the N-[7-benzyloxy-5-fluoro-6-(1,1,4-trioxo-1,2,5-thiadiazolidin-2-yl)-2-naphthyl]-2-(4-bromophenyl)-3-hydroxy-propanamide (5, 0.39 g, 564.71 μmol, 38% yield) as an off-white solid.

LCMS (ES+): m/z 630.0 [M+H]⁺

### Step 4: (2R)—N-[7-benzyloxy-5-fluoro-6-(1,1,4-trioxo-1,2,5-thiadiazolidin-2-yl)-2-naphthyl]-2-[4-[1-(2,6-dibenzyloxy-3-pyridyl)-3-methyl-2-oxo-benzimidazol-5-yl]phenyl]-3-hydroxy-propanamide (7a) and (2S)—N-[7-benzyloxy-5-fluoro-6-(1,1,4-trioxo-1,2,5-thiadiazolidin-2-yl)-2-naphthyl]-2-[4-[1-(2,6-dibenzyloxy-3-pyridyl)-3-methyl-2-oxo-benzimidazol-5-yl]phenyl]-3-hydroxy-propanamide (7b)

Note: Configurations are arbitrarily assigned

Into a 50 mL sealed-tube containing a well-stirred solution of N-[7-benzyloxy-5-fluoro-6-(1,1,4-trioxo-1,2,5-thiadiazolidin-2-yl)-2-naphthyl]-2-(4-bromophenyl)-3-hydroxy-propanamide (5, 390 mg, 564.71 μmol), 1-(2,6-dibenzyloxy-3-pyridyl)-3-methyl-5-(4,4,5,5-tetramethyl-1,3,2-dioxaborolan-2-yl)benzimidazol-2-one (6, 684.27 mg, 1.13 mmol) in 1,4-dioxane (8 mL) and water (2 mL) was added sodium carbonate (179.56 mg, 1.69 mmol) at room temperature. Nitrogen gas was purged through a reaction mixture for 10 min. Then PdCl₂(dppf)·DCM (46.11 mg, 56.47 μmol) was added and the resultant mixture was heated at 90° C. for 5 h. The reaction mixture was acidified with aqueous 1.5N HCl solution and extracted with 15% MeOH in EtOAc (3×50 mL). The combined organic layers were dried over anhydrous Na₂SO₄, filtered and concentrated under reduced pressure to get the crude material that was purified by reverse-phase column chromatography [Silicycle C18 column; Mobile phase A: 10 mM NH₄HCO₃ in water and Mobile phase B: MeCN] to get the 390 mg of N-[7-benzyloxy-5-fluoro-6-(1,1,4-trioxo-1,2,5-thiadiazolidin-2-yl)-2-naphthyl]-2-[4-[1-(2,6-dibenzyloxy-3-pyridyl)-3-methyl-2-oxo-benzimidazol-5-yl]phenyl]-3-hydroxy-propanamide (7a/b).

The isomers were separated by Chiral SFC purification. Method details: Column Name: YMC Cellulose-SB, FlowRate: 5 mL/min, Co-Solvent: 50%, Co-Solvent Name: 0.1% IPAm in MeOH:ACN (1:1), Injected Volume: 15 μL, Temperature: 35° C., Outlet Pressure: 100 bar.

### Chiral SFC:

### Method of Analysis

Instrument: PIC 175

Column: YMC Cellulose SB (250×30) mm, 5 μm

Mobile Phase: CO₂: 0.1% Isopropylamine in Methanol:Acetonitrile (1:1):(55:45)

Total Flow: 100 g/min

Back pressure: 100 bar

Wavelength: 254 nm

Cycle time: 17 min

400 mg of C858155 was dissolved in 6 mL of ACN as well as in Mobile phase and injected 500 μL/injection.

After concentration, the first eluted fraction at RT 3.8 min: (2R)—N-[7-benzyloxy-5-fluoro-6-(1,1,4-trioxo-1,2,5-thiadiazolidin-2-yl)-2-naphthyl]-2-[4-[1-(2,6-dibenzyloxy-3-pyridyl)-3-methyl-2-oxo-benzimidazol-5-yl]phenyl]-3-hydroxy-propanamide (7a, 130 mg, 129.33 μmol, 23% yield) was isolated as an off-white solid.

LCMS (ES+): m/z 985.5 [M+H]⁺

and the second eluted fraction at RT 4.9 min: (2S)—N-[7-benzyloxy-5-fluoro-6-(1,1,4-trioxo-1,2,5-thiadiazolidin-2-yl)-2-naphthyl]-2-[4-[1-(2,6-dibenzyloxy-3-pyridyl)-3-methyl-2-oxo-benzimidazol-5-yl]phenyl]-3-hydroxy-propanamide (7b, 140 mg, 137.86 μmol, 24% yield) was isolated as an off-white solid.

LCMS (ES+): m/z 985.5 [M+H]⁺

### Step 5: (2R)-2-[4-[1-(2,6-dioxo-3-piperidyl)-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]-3-hydroxy-propanamide (Degrader 137)

Into a 10 mL single-neck round-bottom flask containing a well-stirred solution of (2R)—N-[7-benzyloxy-5-fluoro-6-(1,1,4-trioxo-1,2,5-thiadiazolidin-2-yl)-2-naphthyl]-2-[4-[1-(2,6-dibenzyloxy-3-pyridyl)-3-methyl-2-oxo-benzimidazol-5-yl]phenyl]-3-hydroxy-propanamide (7a, 130 mg, 129.33 μmol) in anhydrous 1,4-dioxane (1.5 mL) and DMF (1.5 mL) was added palladium hydroxide on carbon (20 wt. % 50% water) (100 mg, 142.41 μmol, 20% purity) at room temperature. The contents of the flask were stirred at room temperature for 20 h under hydrogen bladder pressure. The reaction mixture was filtered through Celite and concentrated under reduced pressure to get the crude material. Purification by a reverse-phase column chromatography [Silicycle C18 column; Mobile phase A: 0.1% Formic acid in water and Mobile phase B: MeCN]afforded (2R)-2-[4-[1-(2,6-dioxo-3-piperidyl)-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]-3-hydroxy-propanamide (Degrader 137, 70 mg, 95.69 μmol, 74% yield) as an off-white solid.

LCMS (ES−): m/z 715.0 [M−H]⁻

¹H NMR (400 MHz, DMSO) δ 11.12 (s, 1H), 10.38 (s, 1H), 9.85 (s, 1H), 8.21-8.18 (m, 1H), 7.82 (d, J=9.0 Hz, 1H), 7.65 (d, J=8.1 Hz, 2H), 7.51-7.46 (m, 3H), 7.44 (dd, J=9.1, 2.0 Hz, 1H), 7.32 (dd, J=8.2, 1.7 Hz, 1H), 7.18 (d, J=8.3 Hz, 1H), 6.94 (s, 1H), 5.39 (dd, J=12.8, 5.4 Hz, 1H), 4.18-4.07 (m, 3H), 3.98-3.89 (m, 1H), 3.62 (dd, J=10.1, 5.1 Hz, 1H), 3.39 (s, 3H), 3.00-2.85 (m, 1H), 2.80-2.58 (m, 3H), 2.08-1.98 (m, 1H).

### (2S)-2-[4-[1-(2,6-dioxo-3-piperidyl)-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]-3-hydroxy-propanamide (Degrader 138)

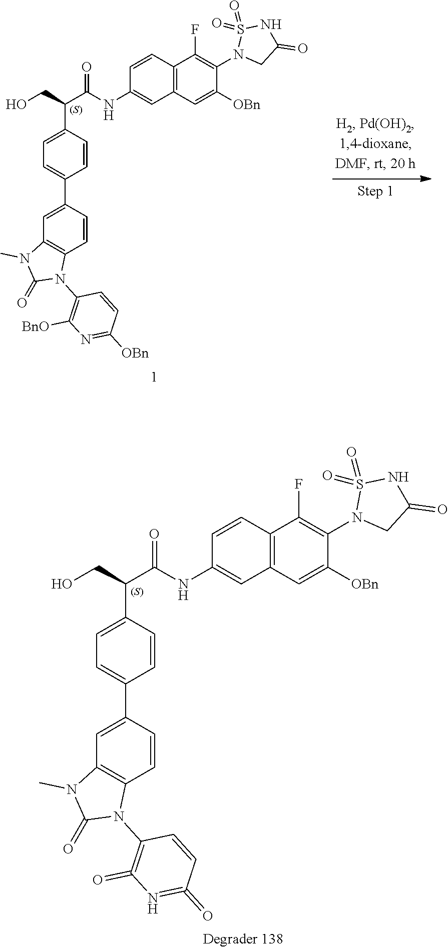

### Step 1: (2S)-2-[4-[1-(2,6-dioxo-3-piperidyl)-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]-3-hydroxy-propanamide (Degrader 138)

Into a 10 mL single-neck round-bottom flask containing a well-stirred solution of (2S)—N-[7-benzyloxy-5-fluoro-6-(1,1,4-trioxo-1,2,5-thiadiazolidin-2-yl)-2-naphthyl]-2-[4-[1-(2,6-dibenzyloxy-3-pyridyl)-3-methyl-2-oxo-benzimidazol-5-yl]phenyl]-3-hydroxy-propanamide (1, 140 mg, 137.86 μmol) in anhydrous 1,4-dioxane (1.5 mL) and DMF (1.5 mL) was added palladium hydroxide on carbon (20 wt. % 50% water, 100 mg, 142.41 μmol, 20% purity) at room temperature. The contents of the flask were stirred at room temperature for 20 h under hydrogen bladder pressure. The reaction mixture was passed through Celite and concentrated under reduced pressure to get the crude material that was purified by reverse-phase column chromatography [Silicycle C18 column; Mobile phase A: 0.1% Formic acid in water and Mobile phase B: MeCN] to afford (2S)-2-[4-[1-(2,6-dioxo-3-piperidyl)-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]-3-hydroxy-propanamide (Degrader 138, 47 mg, 60.85 μmol, 44% yield) as an off-white solid.

LCMS (ES−): m/z 715.0 [M−H]⁻

¹H NMR (400 MHz, DMSO) δ 11.12 (s, 1H), 10.39 (s, 1H), 10.03 (s, 1H), 8.21 (s, 1H), 7.83 (d, J=9.0 Hz, 1H), 7.65 (d, J=8.1 Hz, 2H), 7.52-7.42 (m, 4H), 7.32 (dd, J=8.2, 1.7 Hz, 1H), 7.18 (d, J=8.2 Hz, 1H), 6.95 (s, 1H), 5.39 (dd, J=12.9, 5.4 Hz, 1H), 4.19 (s, 2H), 4.14 (t, J=9.7 Hz, 1H), 3.94 (dd, J=9.4, 5.1 Hz, 1H), 3.62 (dd, J=10.1, 5.1 Hz, 1H), 2.98-2.85 (m, 1H), 2.82-2.58 (m, 3H), 2.09-1.96 (m, 1H).

### 2-[4-[1-(2,6-dioxo-3-piperidyl)-3-methyl-2-oxo-benzimidazol-5-yl]-2-oxo-piperazin-1-yl]-N-[5-fluoro-7-hydroxy-6-(1,1,4-trioxo-1,2,5-thiadiazolidin-2-yl)-2-naphthyl]acetamide (Degrader 139)

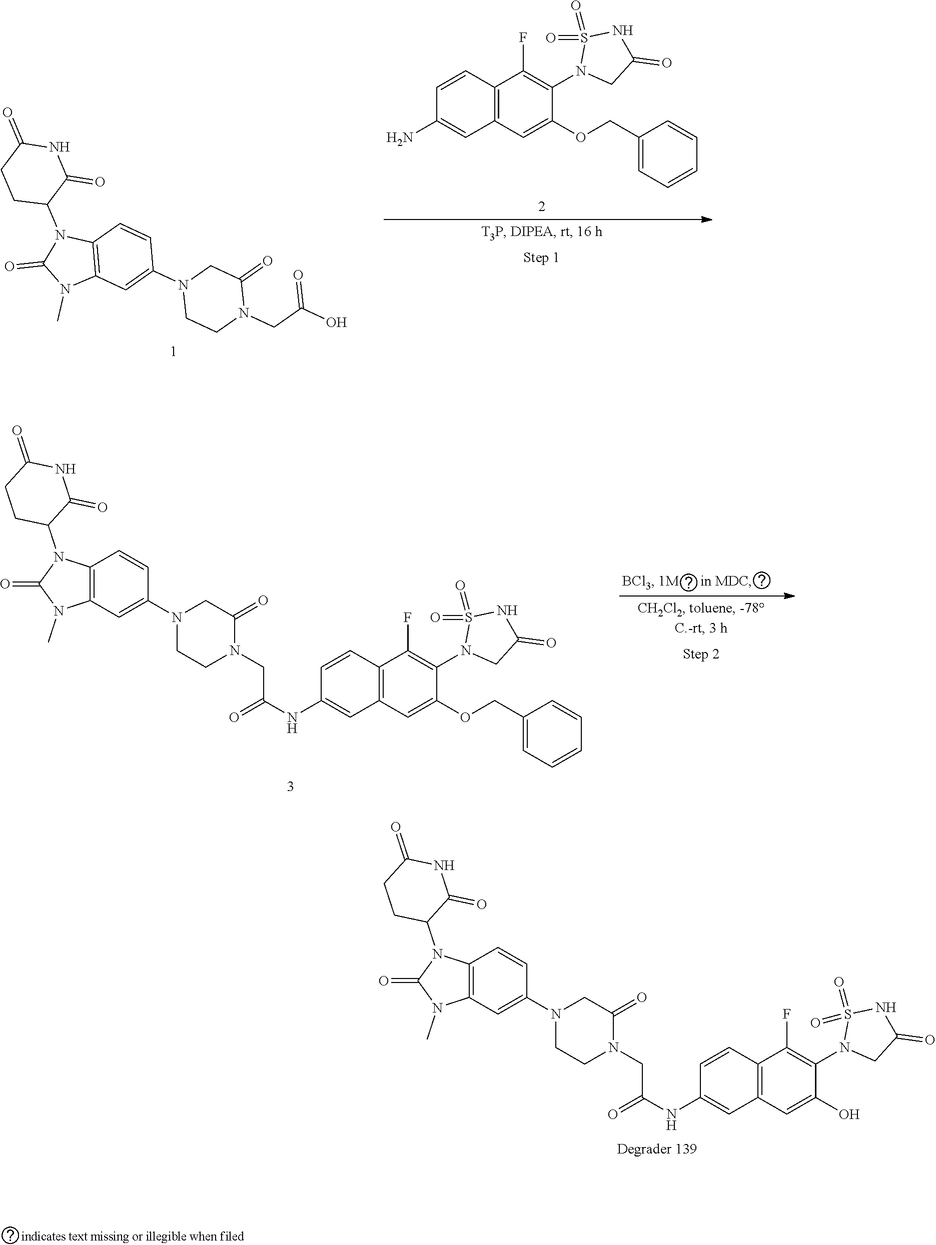 [SMILES: Cn1c(=O)n(C2CCC(=O)NC2=O)c2ccc(N3CCN(CC(=O)O)C(=O)C3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(N3CCN(CC(=O)Nc4ccc5c(F)c(N6CC(=O)NS6(=O)=O)c(OCc6ccccc6)cc5c4)C(=O)C3)cc21 ; Cn1c(=O)n(C2CCC(=O)NC2=O)c2ccc(N3CCN(CC(=O)Nc4ccc5c(F)c(N6CC(=O)NS6(=O)=O)c(O)cc5c4)C(=O)C3)cc21]

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-2-oxo-piperazin-1-yl]acetamide (3)

To a stirred solution of 2-[4-[1-(2,6-dioxo-3-piperidyl)-3-methyl-2-oxo-benzimidazol-5-yl]-2-oxo-piperazin-1-yl]acetic acid (1, 100 mg, 132.09 μmol) in anhydrous DMF (0.5 mL) were added DIPEA (85.36 mg, 660.45 μmol), 5-(6-amino-3-benzyloxy-1-fluoro-2-naphthyl)-1,1-dioxo-1,2,5-thiadiazolidin-3-one (2, 68.08 mg, 132.09 μmol) at 0° C. and 1-propanephosphonic anhydride (50 wt % in ethyl acetate) (252.17 mg, 396.27 μmol, 235.67 uL). After 16 h, the solvent was evaporated and the residue diluted with water (5 mL) and the solid was filtered and dried to give N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-2-oxo-piperazin-1-yl]acetamide (3, 250 mg, 83.91 μmol, 64% yield, 27% purity).

LCMS (ES+): m/z 799.8 [M+H]⁺

### Step 2: 2-[4-[1-(2,6-dioxo-3-piperidyl)-3-methyl-2-oxo-benzimidazol-5-yl]-2-oxo-piperazin-1-yl]-N-[5-fluoro-7-hydroxy-6-(1,1,4-trioxo-1,2,5-thiadiazolidin-2-yl)-2-naphthyl]acetamide (Degrader 139)

To a stirred solution of N-[7-benzyloxy-5-fluoro-6-(1,1,4-trioxo-1,2,5-thiadiazolidin-2-yl)-2-naphthyl]-2-[1-[3-(2,6-dioxo-3-piperidyl)-1-methyl-indazol-6-yl]-4-piperidyl]acetamide (3, 250 mg, 83.91 μmol) in CH₂Cl₂ (10 mL) and toluene (10 mL) was added pentamethylbenzene (62.19 mg, 419.54 mmol) at −78° C. Borontrichloride (1.0 M in methylene chloride) (1.68 mmol, 1.7 mL) was added. The reaction mixture was then stirred at room temperature for 16 h. The reaction was quenched with 5% methanol in CH₂Cl₂ (10 mL) solution and evaporated under vacuum. The residue was washed with MTBE (2×50 mL). The material was purified by reverse phase prep HPLC (Column: XSelect C18 (250×19) mm, 5 micron Mobile phase A: 0.1% ammonium acetate in water; Mobile phase B: MeCN] to give 2-[4-[1-(2,6-dioxo-3-piperidyl)-3-methyl-2-oxo-benzimidazol-5-yl]-2-oxo-piperazin-1-yl]-N-[5-fluoro-7-hydroxy-6-(1,1,4-trioxo-1,2,5-thiadiazolidin-2-yl)-2-naphthyl]acetamide (Degrader 139, 35.26 mg, 49.14 μmol, 59% yield) as a colorless solid.

LCMS (ES+): m/z 709.8 [M+H]⁺

1H-NMR (400 MHz, DMSO-d6): δ 11.14 (s, 1H), 10.33 (s, 1H), 10.01 (s, 1H), 8.17 (s, 1H), 7.88 (d, J=9.20 Hz, 1H), 7.55 (d, J=9.20 Hz, 1H), 7.21-7.16 (m, 2H), 7.02-6.96 (m, 3H), 5.43-5.38 (m, 1H), 4.19 (s, 2H), 3.79-3.38 (m, 5H), 2.95-2.88 (m, 1H), 2.78-2.68 (m, 3H), 2.51-2.51 (m, 1H), 2.09-2.02 (m, 1H).

Note: Not some of the protons are merged in solvent signals.

### 2-[3-[1-(2,6-dioxo-3-piperidyl)-3-methyl-2-oxo-benzimidazol-5-yl]-4-fluoro-1-piperidyl]-N-[5-fluoro-7-hydroxy-6-(1,1,4-trioxo-1,2,5-thiadiazolidin-2-yl)-2-naphthyl]acetamide (Degrader 140)

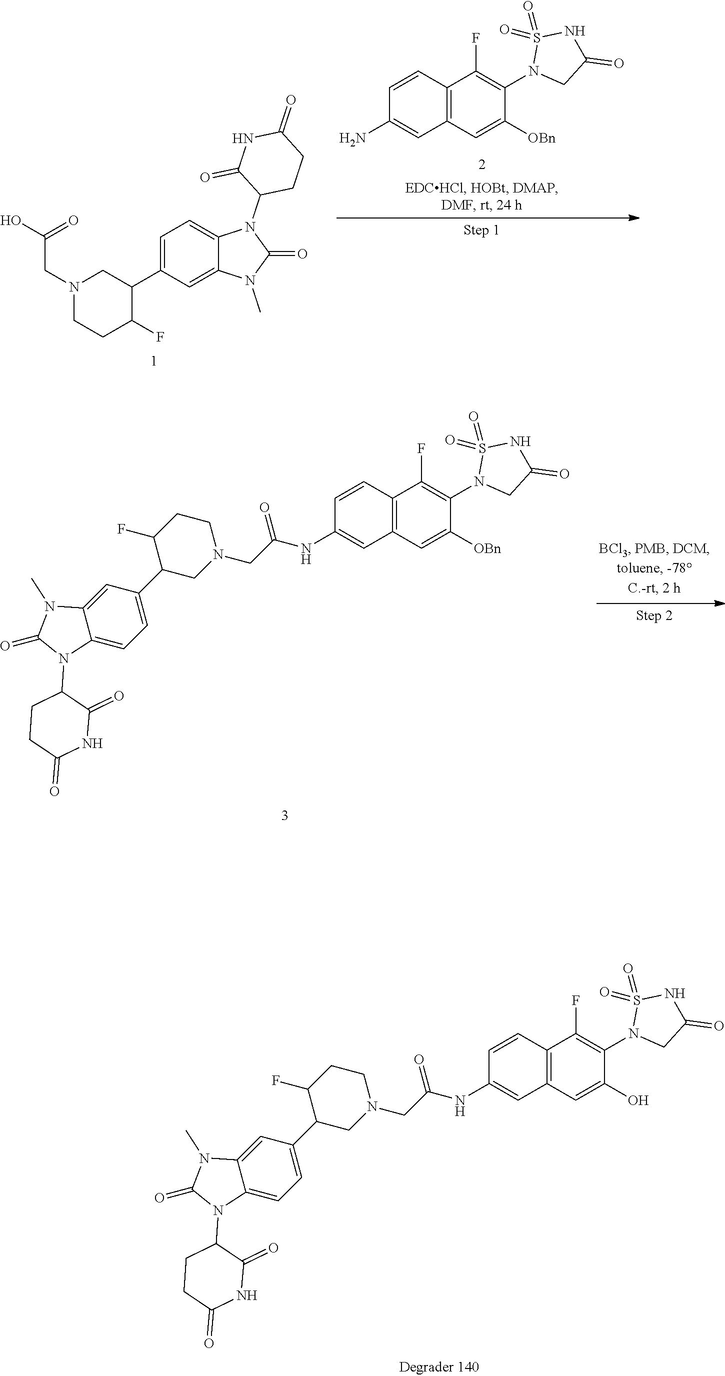 [SMILES: Cn1c(=O)n(C2CCC(=O)NC2=O)c2ccc(C3CN(CC(=O)O)CCC3F)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(C3CN(CC(=O)Nc4ccc5c(F)c(N6CC(=O)NS6(=O)=O)c(OCc6ccccc6)cc5c4)CCC3F)cc21 ; Cn1c(=O)n(C2CCC(=O)NC2=O)c2ccc(C3CN(CC(=O)Nc4ccc5c(F)c(N6CC(=O)NS6(=O)=O)c(O)cc5c4)CCC3F)cc21]

### Step 1: N-[7-benzyloxy-5-fluoro-6-(1,1,4-trioxo-1,2,5-thiadiazolidin-2-yl)-2-naphthyl]-2-[3-[1-(2,6-dioxo-3-piperidyl)-3-methyl-2-oxo-benzimidazol-5-yl]-4-fluoro-1-piperidyl]acetamide (3)

Into a 100 mL round bottom flask containing a well-stirred solution of 2-[3-[1-(2,6-dioxo-3-piperidyl)-3-methyl-2-oxo-benzimidazol-5-yl]-4-fluoro-1-piperidyl]acetic acid (1, 150 mg, 264.82 μmol, TFA salt) in anhydrous DMF (0.5 mL) was added 5-(6-amino-3-benzyloxy-1-fluoro-2-naphthyl)-1,1-dioxo-1,2,5-thiadiazolidin-3-one (2, 101.97 mg, 185.37 μmol), HOBt (71.56 mg, 529.64 μmol), EDC (152.30 mg, 794.45 μmol) and DMAP (194.12 mg, 1.59 mmol). After 16 h, the solvent was removed under reduced pressure and the residue was suspended in 1.5N HCl (1.5 mL). The solid was filtered and washed with water and diethyl ether and dried under vacuum to afford N-[7-benzyloxy-5-fluoro-6-(1,1,4-trioxo-1,2,5-thiadiazolidin-2-yl)-2-naphthyl]-2-[3-[1-(2,6-dioxo-3-piperidyl)-3-methyl-2-oxo-benzimidazol-5-yl]-4-fluoro-1-piperidyl]acetamide (3, 90 mg, 96.88 μmol, 37% yield) as an off-white solid.

LCMS (ES−): m/z 800.0 [M−H]⁻

### Step 2: 2-[3-[1-(2,6-dioxo-3-piperidyl)-3-methyl-2-oxo-benzimidazol-5-yl]-4-fluoro-1-piperidyl]-N-[5-fluoro-7-hydroxy-6-(1,1,4-trioxo-1,2,5-thiadiazolidin-2-yl)-2-naphthyl]acetamide (Degrader 140)

Into a 50 mL round bottom flask containing a well-stirred solution of N-[7-benzyloxy-5-fluoro-6-(1,1,4-trioxo-1,2,5-thiadiazolidin-2-yl)-2-naphthyl]-2-[3-[1-(2,6-dioxo-3-piperidyl)-3-methyl-2-oxo-benzimidazol-5-yl]-4-fluoro-1-piperidyl]acetamide (3, 90 mg, 96.53 μmol) in toluene (1 mL) and DCM (1 mL) was added pentamethylbenzene (71.55 mg, 482.66 μmol, 78.03 μL) and the reaction mixture was cooled to −78° C. Then boron trichloride (1.0 M in dichloromethane) (1.93 mmol, 1.93 mL) was added dropwise. The reaction mixture was stirred at room temperature for 3 h. The reaction mixture was cooled to −78° C. and quenched slowly with 5% methanol in dichloromethane (1 mL). The reaction mixture was concentrated under reduced pressure and the residue was washed with diethyl ether (10 mL) and filtered. The crude compound was purified by reverse phase prep HPLC [Purification method: Column: X-Bridge C18 (150×19) mm, 5 micron; Mobile phase A: 0.1% TFA in water and Mobile phase B: MeCN] to afford 2-[3-[1-(2,6-dioxo-3-piperidyl)-3-methyl-2-oxo-benzimidazol-5-yl]-4-fluoro-1-piperidyl]-N-[5-fluoro-7-hydroxy-6-(1,1,4-trioxo-1,2,5-thiadiazolidin-2-yl)-2-naphthyl]acetamide (Degrader 140, 25 mg, 29.73 μmol, 31% yield, TFA Salt) as a colorless solid.

LCMS (ES+): m/z 712.0 [M+H]⁺

1H-NMR (400 MHz, DMSO-d6): δ 11.12 (s, 1H), 10.72 (bs, 1H), 10.34 (bs, 1H), 9.90 (bs, 1H), 8.10 (s, 1H), 7.89 (d, J=9.20 Hz, 1H), 7.45 (d, J=8.80 Hz, 1H), 7.24 (s, 1H), 7.14 (d, J=8.00 Hz, 1H), 7.04 (d, J=8.00 Hz, 1H), 6.99 (s, 1H), 5.40-5.36 (m, 1H), 5.12-4.99 (bm, 1H), 4.15-4.11 (m, 4H), 3.71-3.55 (m, 4H), 2.94-2.87 (m, 1H), 2.76-2.66 (m, 3H), 2.39-2.33 (m, 1H), 2.23-2.00 (m, 2H).

### 2-[(3R,4R)-3-[1-(2,6-dioxo-3-piperidyl)-3-methyl-2-oxo-benzimidazol-5-yl]-4-fluoro-1-piperidyl]-N-[5-fluoro-7-hydroxy-6-(1,1,4-trioxo-1,2,5-thiadiazolidin-2-yl)-2-naphthyl]acetamide (Degrader 141)

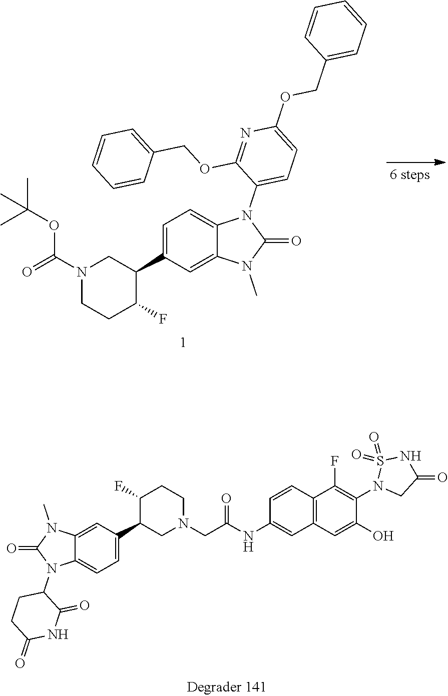 [SMILES: Cn1c(=O)n(-c2ccc(OCc3ccccc3)nc2OCc2ccccc2)c2ccc([C@@H]3CN(C(=O)OC(C)(C)C)CC[C@H]3F)cc21 ; Cn1c(=O)n(C2CCC(=O)NC2=O)c2ccc([C@@H]3CN(CC(=O)Nc4ccc5c(F)c(N6CC(=O)NS6(=O)=O)c(O)cc5c4)CC[C@H]3F)cc21]

2-[(3R,4R)-3-[1-(2,6-dioxo-3-piperidyl)-3-methyl-2-oxo-benzimidazol-5-yl]-4-fluoro-1-piperidyl]-N-[5-fluoro-7-hydroxy-6-(1,1,4-trioxo-1,2,5-thiadiazolidin-2-yl)-2-naphthyl]acetamide (Degrader 141, 35 mg, 45.40 μmol, formic acid salt) was synthesized from tert-butyl (3R,4R)-3-[1-(2,6-dibenzyloxy-3-pyridyl)-3-methyl-2-oxo-benzimidazol-5-yl]-4-fluoro-piperidine-1-carboxylate (1) following the same six step procedure as 2-[3-[1-(2,6-dioxo-3-piperidyl)-3-methyl-2-oxo-benzimidazol-5-yl]-4-fluoro-1-piperidyl]-N-[5-fluoro-7-hydroxy-6-(1,1,4-trioxo-1,2,5-thiadiazolidin-2-yl)-2-naphthyl]acetamide (Degrader 140)

LCMS (ES+): m/z 711.9 [M+H]⁺

1H-NMR (400 MHz, DMSO-d6): δ 11.12 (s, 1H), 10.70 (bs, 1H), 10.30 (bs, 1H), 9.91 (s, 1H), 8.11 (s, 1H), 7.89 (d, J=8.80 Hz, 1H), 7.45 (d, J=8.40 Hz, 1H), 7.24 (s, 1H), 7.14 (d, J=8.00 Hz, 1H), 7.04 (d, J=7.60 Hz, 1H), 6.99 (s, 1H), 5.40-5.36 (m, 1H), 5.12-5.00 (broad m, 1H), 4.15-4.11 (m, 4H), 3.71-3.55 (m, 4H), 2.94-2.87 (m, 1H), 2.76-2.61 (m, 3H), 2.50-2.34 (m, 1H), 2.34-2.09 (m, 2H).

### 2-[(3S,4S)-3-[1-(2,6-dioxo-3-piperidyl)-3-methyl-2-oxo-benzimidazol-5-yl]-4-fluoro-1-piperidyl]-N-[5-fluoro-7-hydroxy-6-(1,1,4-trioxo-1,2,5-thiadiazolidin-2-yl)-2-naphthyl]acetamide (Degrader 142)

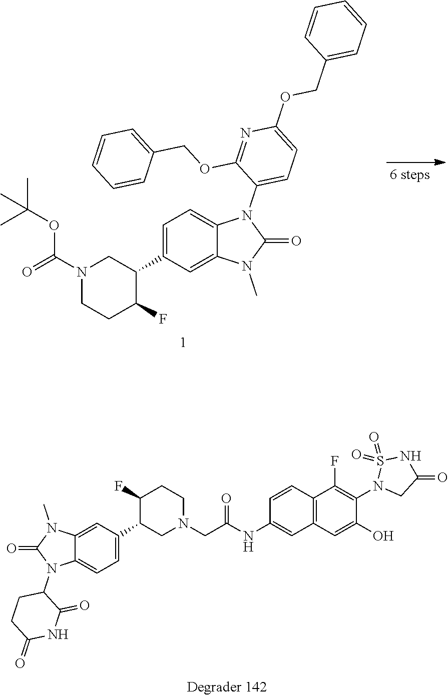 [SMILES: Cn1c(=O)n(-c2ccc(OCc3ccccc3)nc2OCc2ccccc2)c2ccc([C@H]3CN(C(=O)OC(C)(C)C)CC[C@@H]3F)cc21 ; Cn1c(=O)n(C2CCC(=O)NC2=O)c2ccc([C@H]3CN(CC(=O)Nc4ccc5c(F)c(N6CC(=O)NS6(=O)=O)c(O)cc5c4)CC[C@@H]3F)cc21]

2-[(3S,4S)-3-[1-(2,6-dioxo-3-piperidyl)-3-methyl-2-oxo-benzimidazol-5-yl]-4-fluoro-1-piperidyl]-N-[5-fluoro-7-hydroxy-6-(1,1,4-trioxo-1,2,5-thiadiazolidin-2-yl)-2-naphthyl]acetamide (Degrader 142, 25 mg, 32.86 μmol, formic acid salt) was synthesized from tert-butyl (3S,4S)-3-[1-(2,6-dibenzyloxy-3-pyridyl)-3-methyl-2-oxo-benzimidazol-5-yl]-4-fluoro-piperidine-1-carboxylate (1) following the same six step procedure as 2-[3-[1-(2,6-dioxo-3-piperidyl)-3-methyl-2-oxo-benzimidazol-5-yl]-4-fluoro-1-piperidyl]-N-[5-fluoro-7-hydroxy-6-(1,1,4-trioxo-1,2,5-thiadiazolidin-2-yl)-2-naphthyl]acetamide (Degrader 140).

LCMS (ES+): m/z 712.0 [M+H]⁺

1H-NMR (400 MHz, DMSO-d6): δ 11.12 (s, 1H), 10.72 (bs, 1H), 10.33 (bs, 1H), 9.89 (s, 1H), 8.11 (s, 1H), 7.88 (d, J=9.20 Hz, 1H), 7.46 (d, J=8.00 Hz, 1H), 7.24 (s, 1H), 7.14 (d, J=8.00 Hz, 1H), 7.04 (d, J=8.00 Hz, 1H), 6.98 (s, 1H), 5.40-5.36 (broad m, 1H), 5.14-4.97 (m, 1H), 4.15-4.10 (m, 4H), 3.71-3.55 (m, 4H), 2.95-2.87 (m, 1H), 2.77-2.61 (m, 3H), 2.50-2.34 (m, 1H), 2.35-2.09 (m, 2H).

### 2-[1-[1-(2,6-dioxo-3-piperidyl)-2-oxo-benzo[cd]indol-6-yl]-4-piperidyl]-N-[5-fluoro-7-hydroxy-6-(1,1,4-trioxo-1,2,5-thiadiazolidin-2-yl)-2-naphthyl]acetamide (Degrader 143)

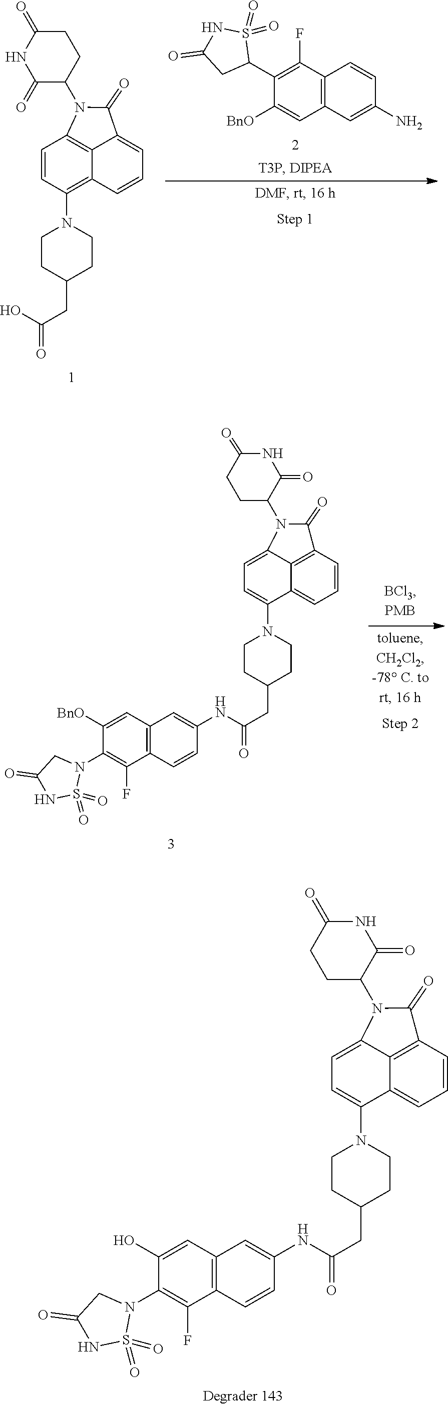 [SMILES: O=C(O)CC1CCN(c2ccc3c4c(cccc24)C(=O)N3C2CCC(=O)NC2=O)CC1 ; Nc1ccc2c(F)c(C3CC(=O)NS3(=O)=O)c(OCc3ccccc3)cc2c1 ; O=C1CCC(N2C(=O)c3cccc4c(N5CCC(CC(=O)Nc6ccc7c(F)c(N8CC(=O)NS8(=O)=O)c(OCc8ccccc8)cc7c6)CC5)ccc2c34)C(=O)N1 ; O=C1CCC(N2C(=O)c3cccc4c(N5CCC(CC(=O)Nc6ccc7c(F)c(N8CC(=O)NS8(=O)=O)c(O)cc7c6)CC5)ccc2c34)C(=O)N1]

### Step 1: N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-2-oxo-benzo[cd]indol-6-yl]-4-piperidyl]acetamide (3)

To a stirred solution of 2-[1-[1-(2,6-dioxo-3-piperidyl)-2-oxo-benzo[cd]indol-6-yl]-4-piperidyl]acetic acid (1, 300 mg, 530.32 μmol) in DMF (5 mL) was added i-Pr₂NEt (342.70 mg, 2.65 mmol, 461.86 μL) and 5-(6-amino-3-benzyloxy-1-fluoro-2-naphthyl)-1,1-dioxo-1,2,5-thiadiazolidin-3-one (2, 212.88 mg, 413.00 μmol, TFA salt) at 0° C. 1-propanephosphonic anhydride (50 wt % in ethyl acetate) (1.01 g, 1.59 mmol, 946.19 μL) was added and stirring was continued at 25° C. for 16 h. The reaction mixture was concentrated and then ice-cold water (5 mL) was added. The precipitate was filtered and dried to afford N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-2-oxo-benzo[cd]indol-6-yl]-4-piperidyl]acetamide (3, 350 mg, 134.33 μmol, 25% yield, 31% purity).

The material was used directly in the next step.

LCMS (ES+): m/z 805.2 [M+H]⁺

### Step 2: 2-[1-[1-(2,6-dioxo-3-piperidyl)-2-oxo-benzo[cd]indol-6-yl]-4-piperidyl]-N-[5-fluoro-7-hydroxy-6-(1,1,4-trioxo-1,2,5-thiadiazolidin-2-yl)-2-naphthyl]acetamide (Degrader 143)

To a stirred solution of N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-2-oxo-benzo[cd]indol-6-yl]-4-piperidyl]acetamide (3, 250 mg, 105.61 μmol) and pentamethylbenzene (78.28 mg, 528.05 μmol, 85.37 μL) in toluene (3 mL) and CH₂Cl₂ (3 mL) under nitrogen atmosphere at −78° C. was added BCl₃ (1.0 M in DCM) (2.11 mmol, 2.11 mL). The mixture was stirred for 16 h at 25° C. The reaction was quenched with 5% MeOH in DCM (10 mL), concentrated and purified by reverse-phase preparatory HPLC [Column: X-select C18 (250×19) mm 5 micron; Mobile Phase A: 0.1% ammonium acetate in water and Mobile Phase B: ACN] to afford 2-[1-[1-(2,6-dioxo-3-piperidyl)-2-oxo-benzo[cd]indol-6-yl]-4-piperidyl]-N-[5-fluoro-7-hydroxy-6-(1,1,4-trioxo-1,2,5-thiadiazolidin-2-yl)-2-naphthyl]acetamide (Degrader 143, 35.96 mg, 49.25 μmol, 47% yield) as an orange solid.

LCMS (ES+): m/z 714.8 [M+H]⁺

¹H-NMR (400 MHz, DMSO-d6): δ 11.11 (s, 1H), 10.17 (s, 1H), 8.25 (d, J=8.40 Hz, 1H), 8.19 (s, 1H), 8.08 (d, J=7.20 Hz, 1H), 7.85-7.81 (m, 2H), 7.45 (dd, J=1.20, 9.20 Hz, 1H), 7.02 (d, J=8.00 Hz, 1H), 6.97-6.95 (m, 2H), 5.42 (dd, J=5.20, 12.80 Hz, 1H), 4.07 (s, 2H), 3.46-3.43 (m, 2H), 3.01-2.90 (m, 1H), 2.85-2.72 (m, 3H), 2.45 (d, J=6.80 Hz, 2H), 2.13-1.96 (m, 2H), 1.93-1.90 (m, 2H), 1.66-1.63 (m, 2H).

Note: 1 aliphatic proton is obscured by the solvent signal.

### 1-[2-[1-[1-(2,6-dioxo-3-piperidyl)-4-fluoro-3-methyl-2-oxo-benzimidazol-5-yl]-4-piperidyl]ethyl]-3-[5-fluoro-7-hydroxy-6-(1,1,4-trioxo-1,2,5-thiadiazolidin-2-yl)-2-naphthyl]urea (Degrader 144)

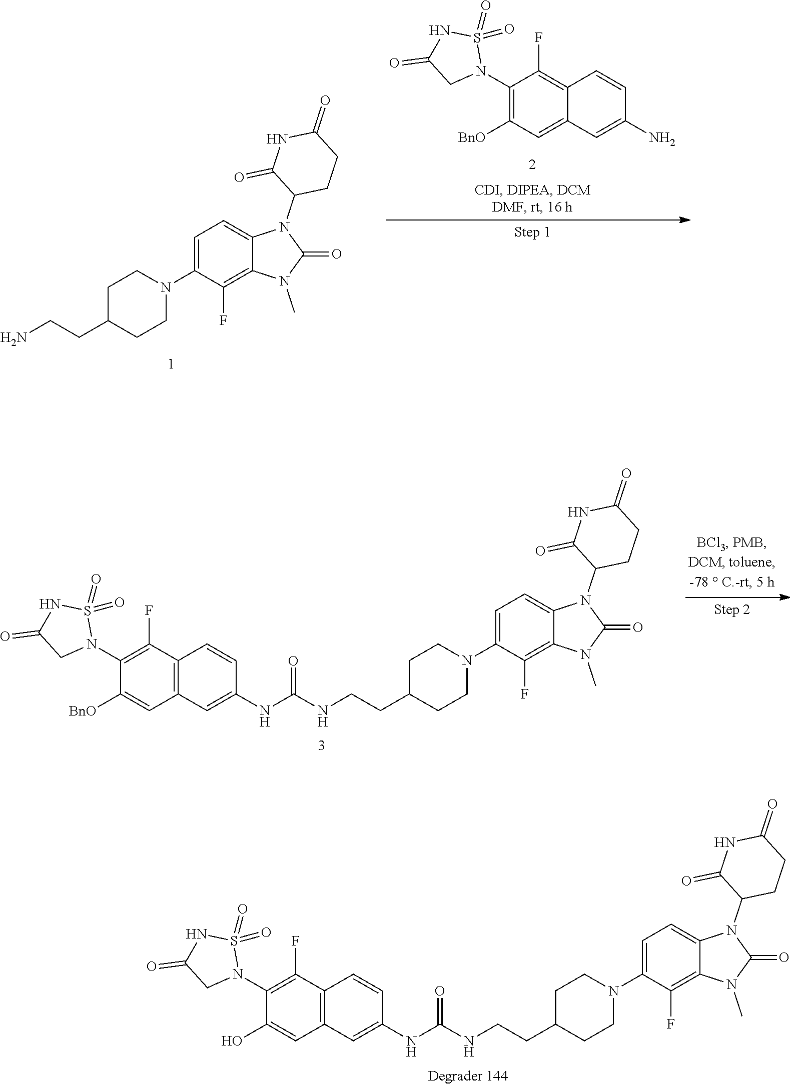 [SMILES: Cn1c(=O)n(C2CCC(=O)NC2=O)c2ccc(N3CCC(CCN)CC3)c(F)c21 ; Nc1ccc2c(F)c(N3CC(=O)NS3(=O)=O)c(OCc3ccccc3)cc2c1 ; Cn1c(=O)n(C2CCC(=O)NC2=O)c2ccc(N3CCC(CCNC(=O)Nc4ccc5c(F)c(N6CC(=O)NS6(=O)=O)c(OCc6ccccc6)cc5c4)CC3)c(F)c21 ; Cn1c(=O)n(C2CCC(=O)NC2=O)c2ccc(N3CCC(CCNC(=O)Nc4ccc5c(F)c(N6CC(=O)NS6(=O)=O)c(O)cc5c4)CC3)c(F)c21]

### Step 1: 1-[7-benzyloxy-5-fluoro-6-(1,1,4-trioxo-1,2,5-thiadiazolidin-2-yl)-2-naphthyl]-3-[2-[1-[1-(2,6-dioxo-3-piperidyl)-4-fluoro-3-methyl-2-oxo-benzimidazol-5-yl]-4-piperidyl]ethyl]urea (3)

Into a 50 mL single neck round bottom flask containing a well-stirred solution of 5-(6-amino-3-benzyloxy-1-fluoro-2-naphthyl)-1,1-dioxo-1,2,5-thiadiazolidin-3-one (2, 100 mg, 190.13 μmol) in anhydrous DCM (2 mL) and DMF (1 mL) were added DIPEA (122.86 mg, 950.66 μmol, 165.58 μL) and CDI (54.74 mg, 380.26 μmol). After 2 h, a solution of 3-[5-[4-(2-aminoethyl)-1-piperidyl]-4-fluoro-3-methyl-2-oxo-benzimidazol-1-yl]piperidine-2,6-dione (1, 87.20 mg, 190.13 μmol) in DMF (1 mL) and DIPEA (122.86 mg, 950.66 μmol, 165.58 μL) was added. After 16 h, the solvent was removed under reduced pressure and acidified with 1.5N HCl (3 mL). The precipitate was filtered and dried to afford 1-[7-benzyloxy-5-fluoro-6-(1,1,4-trioxo-1,2,5-thiadiazolidin-2-yl)-2-naphthyl]-3-[2-[1-[1-(2,6-dioxo-3-piperidyl)-4-fluoro-3-methyl-2-oxo-benzimidazol-5-yl]-4-piperidyl]ethyl]urea (3, 90 mg, 44.03 μmol, 23% yield, HCL salt) as a brownish solid.

LCMS (ES+): m/z 831.0 [M+H]⁺

### Step 2: 1-[2-[1-[1-(2,6-dioxo-3-piperidyl)-4-fluoro-3-methyl-2-oxo-benzimidazol-5-yl]-4-piperidyl]ethyl]-3-[5-fluoro-7-hydroxy-6-(1,1,4-trioxo-1,2,5-thiadiazolidin-2-yl)-2-naphthyl]urea (Degrader 144)

Into a 50 mL single neck round bottom flask containing a well-stirred solution of 1-[7-benzyloxy-5-fluoro-6-(1,1,4-trioxo-1,2,5-thiadiazolidin-2-yl)-2-naphthyl]-3-[2-[1-[1-(2,6-dioxo-3-piperidyl)-4-fluoro-3-methyl-2-oxo-benzimidazol-5-yl]-4-piperidyl]ethyl]urea (3, 190 mg, 91.47 μmol) in DCM (2.5 mL) and toluene (2.5 mL) was added pentamethylbenzene (67.80 mg, 457.36 μmol). Then boron trichloride (1.0 M solution in methylene chloride) (1.83 mmol, 1.83 mL) was added at −78° C. The reaction mixture was stirred at room temperature for 5 h. The reaction mixture was cooled to −78° C. and was quenched with 5% Methanol in DCM (2 mL). The solvent was removed and washed with MTBE (10 mL) to afford the crude compound which was purified by reverse phase prep HPLC [Purification method: Column: XSelect C18 (250×19) mm, 5 microns, Mobile phase A: 0.1% TFA in water; Mobile phase B: MeCN] to afford 1-[2-[1-[1-(2,6-dioxo-3-piperidyl)-4-fluoro-3-methyl-2-oxo-benzimidazol-5-yl]-4-piperidyl]ethyl]-3-[5-fluoro-7-hydroxy-6-(1,1,4-trioxo-1,2,5-thiadiazolidin-2-yl)-2-naphthyl]urea (Degrader 144, 35 mg, 40.31 μmol, 44% yield, TFA salt) as an off-white solid.

LCMS (ES+): m/z 741.0 [M+H]⁺

¹H-NMR (400 MHz, DMSO-d6): δ 11.11 (s, 1H), 10.38 (bs, 1H), 8.74 (s, 1H), 7.93 (s, 1H), 7.78 (d, J=8.80 Hz, 1H), 7.28 (dd, J=2.00, 9.00 Hz, 1H), 6.90-6.86 (m, 2H), 6.81-6.77 (m, 1H), 6.29 (s, 1H), 5.36-5.32 (m, 1H), 4.43 (s, 2H), 3.48 (s, 3H), 3.48-3.20 (m, 5H), 2.92-2.85 (m, 1H), 2.73-2.64 (m, 4H), 2.02-1.99 (m, 1H), 1.84-1.81 (m, 2H), 1.48-1.37 (m, 5H).

### 4-[3-(2,6-dioxo-3-piperidyl)-1-methyl-2-oxo-8,9-dihydro-7H-imidazo[4,5-f]quinolin-6-yl]-N-[5-fluoro-7-hydroxy-6-(1,1,4-trioxo-1,2,5-thiadiazolidin-2-yl)-2-naphthyl]butanamide (Degrader 145)

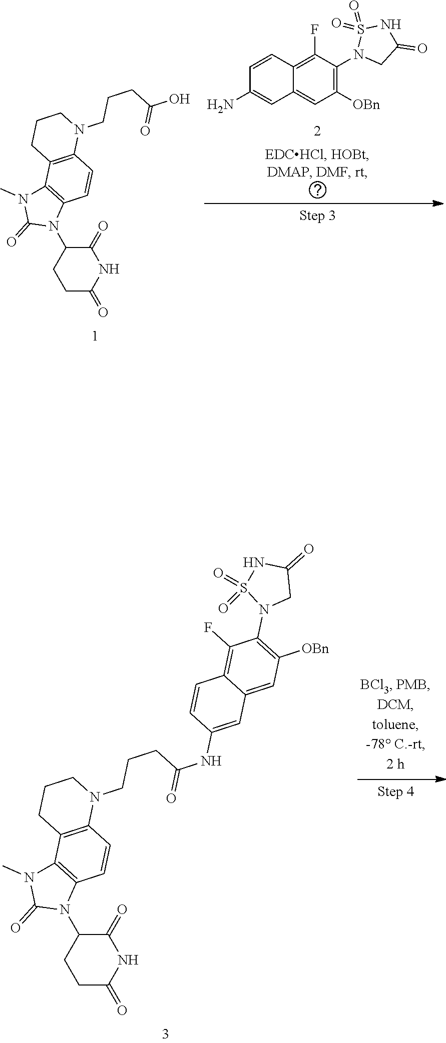 [SMILES: Cn1c(=O)n(C2CCC(=O)NC2=O)c2ccc3c(c21)CCCN3CCCC(=O)O ; Nc1ccc2c(F)c(N3CC(=O)NS3(=O)=O)c(OCc3ccccc3)cc2c1 ; Cn1c(=O)n(C2CCC(=O)NC2=O)c2ccc3c(c21)CCCN3CCCC(=O)Nc1ccc2c(F)c(N3CC(=O)NS3(=O)=O)c(OCc3ccccc3)cc2c1]

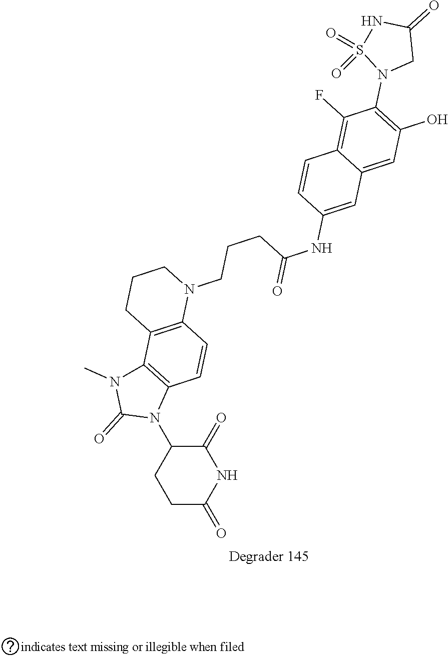 [SMILES: Cn1c(=O)n(C2CCC(=O)NC2=O)c2ccc3c(c21)CCCN3CCCC(=O)Nc1ccc2c(F)c(N3CC(=O)NS3(=O)=O)c(O)cc2c1]

### Step 1: N-[7-benzyloxy-5-fluoro-6-(1,1,4-trioxo-1,2,5-thiadiazolidin-2-yl)-2-naphthyl]-4-[3-(2,6-dioxo-3-piperidyl)-1-methyl-2-oxo-8,9-dihydro-7H-imidazo[4,5-f]quinolin-6-yl]butanamide (3)

Into a 20 mL vial containing a well-stirred solution of 5-(6-amino-3-benzyloxy-1-fluoro-2-naphthyl)-1,1-dioxo-1,2,5-thiadiazolidin-3-one (2, 110 mg, 211.28 Mol, TFA salt) in DMF (2 mL) were added 4-[3-(2,6-dioxo-3-piperidyl)-1-methyl-2-oxo-8,9-dihydro-7H-imidazo[4,5-f]quinolin-6-yl]butanoic acid (1, 114.41 mg, 211.28 μmol, TFA salt), N-(3-dimethylaminopropyl)-N′-ethylcarbodiimide hydrochloride (121.51 mg, 633.84 μmol), HOBT (57.10 mg, 422.56 μmol) and DMAP (154.87 mg, 1.27 mmol). After 16 h, the solvent was removed under reduced pressure and acidified with 1.5N HCl. The solid was filtered, washed with water and dried under vacuum to afford N-[7-benzyloxy-5-fluoro-6-(1,1,4-trioxo-1,2,5-thiadiazolidin-2-yl)-2-naphthyl]-4-[3-(2,6-dioxo-3-piperidyl)-1-methyl-2-oxo-8,9-dihydro-7H-imidazo[4,5-J]quinolin-6-yl]butanamide (3, 190 mg, 160.47 μmol, 76% yield, 66% purity) as an brown solid. The material was used in the next step without further purification.

LCMS (ES+): m/z 784.0 [M+H]⁺

### Step 4: 4-[3-(2,6-dioxo-3-piperidyl)-1-methyl-2-oxo-8,9-dihydro-7H-imidazo[4,5-f]quinolin-6-yl]-N-[5-fluoro-7-hydroxy-6-(1,1,4-trioxo-1,2,5-thiadiazolidin-2-yl)-2-naphthyl]butanamide (Degrader 145)

Into a 50 mL round bottom flask containing a well-stirred solution of N-[7-benzyloxy-5-fluoro-6-(1,1,4-trioxo-1,2,5-thiadiazolidin-2-yl)-2-naphthyl]-4-[3-(2,6-dioxo-3-piperidyl)-1-methyl-2-oxo-8,9-dihydro-7H-imidazo[4,5-f]quinolin-6-yl]butanamide (3, 190 mg, 160.47 μmol) in toluene (2.5 mL) and DCM (2.5 mL) was added pentamethylbenzene (118.94 mg, 802.35 μmol) and the reaction mixture was cooled to −78° C. Then boron trichloride (1.0 M in dichloromethane) (3.21 mmol, 3.2 mL) was added dropwise over a period of 2 min. The reaction mixture was brought to room temperature and stirred for 3 h. The reaction mixture was cooled to −78° C. and quenched with 5% methanol in dichloromethane (1 mL). The reaction mixture was concentrated under reduced pressure and the residue was washed with MTBE (10 mL) and filtered. The material was purified by reverse phase column chromatography [Silicycle C18 column; Mobile phase A: 0.1% formic acid in water; Mobile phase B: MeCN] to afford 4-[3-(2,6-dioxo-3-piperidyl)-1-methyl-2-oxo-8,9-dihydro-7H-imidazo[4,5-f]quinolin-6-yl]-N-[5-fluoro-7-hydroxy-6-(1,1,4-trioxo-1,2,5-thiadiazolidin-2-yl)-2-naphthyl]butanamide (Degrader 145, 65 mg, 86.18 μmol, 54% yield, formic acid salt) as a colorless solid.

LCMS (ES+): m/z 694.0 [M+H]⁺

¹H-NMR (400 MHz, DMSO-d6): δ 11.11 (s, 1H), 10.29 (s, 1H), 10.17 (s, 1H), 8.17 (s, 1H), 7.84 (s, 1H), 7.43 (dd, J=1.60, 9.20 Hz, 1H), 6.96 (s, 1H), 6.79 (d, J=7.20 Hz, 1H), 6.52 (d, J=7.20 Hz, 1H), 5.27-5.24 (m, 1H), 4.34 (s, 2H), 3.32-3.15 (m, 8H), 2.88-2.84 (m, 1H), 2.71-2.55 (m, 3H), 2.09-2.03 (m, 5H).

### 5-[3-(2,6-dioxo-3-piperidyl)-1-methyl-2-oxo-8,9-dihydro-7H-imidazo[4,5-f]quinolin-6-yl]-N-[5-fluoro-7-hydroxy-6-(1,1,4-trioxo-1,2,5-thiadiazolidin-2-yl)-2-naphthyl]pentanamide (Degrader 146)

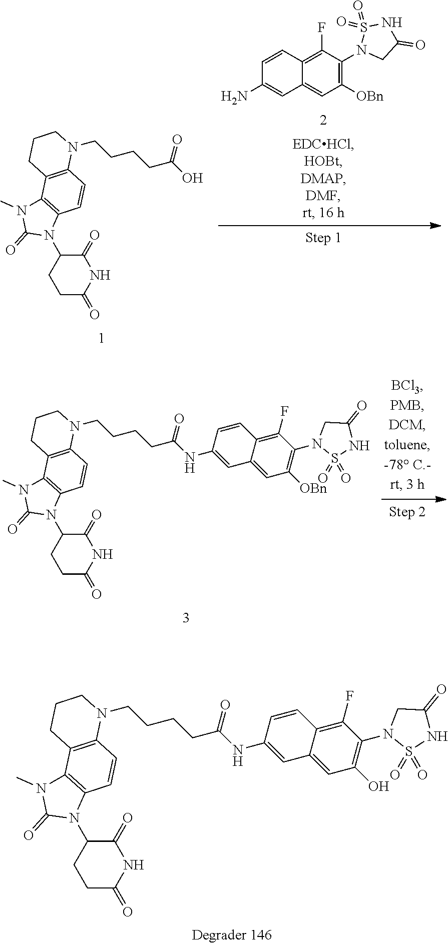 [SMILES: Nc1ccc2c(F)c(N3CC(=O)NS3(=O)=O)c(OCc3ccccc3)cc2c1 ; Cn1c(=O)n(C2CCC(=O)NC2=O)c2ccc3c(c21)CCCN3CCCCC(=O)O ; Cn1c(=O)n(C2CCC(=O)NC2=O)c2ccc3c(c21)CCCN3CCCCC(=O)Nc1ccc2c(F)c(N3CC(=O)NS3(=O)=O)c(OCc3ccccc3)cc2c1 ; Cn1c(=O)n(C2CCC(=O)NC2=O)c2ccc3c(c21)CCCN3CCCCC(=O)Nc1ccc2c(F)c(N3CC(=O)NS3(=O)=O)c(O)cc2c1]

5-[3-(2,6-dioxo-3-piperidyl)-1-methyl-2-oxo-8,9-dihydro-7H-imidazo[4,5-f]quinolin-6-yl]-N-[5-fluoro-7-hydroxy-6-(1,1,4-trioxo-1,2,5-thiadiazolidin-2-yl)-2-naphthyl]pentanamide (Degrader 146, 47 mg, 60.18 μmol, formic acid salt) was synthesized following the same two step procedure as 4-[3-(2,6-dioxo-3-piperidyl)-1-methyl-2-oxo-8,9-dihydro-7H-imidazo[4,5-f]quinolin-6-yl]-N-[5-fluoro-7-hydroxy-6-(1,1,4-trioxo-1,2,5-thiadiazolidin-2-yl)-2-naphthyl]butanamide (Degrader 145).

LCMS (ES+): m/z 708.0 [M+H]⁺

¹H-NMR (400 MHz, DMSO-d6): δ 11.06 (s, 1H), 10.22 (s, 1H), 10.15 (s, 1H), 8.17 (s, 1H), 7.84 (d, J=9.20 Hz, 1H), 7.42 (dd, J=1.60, 9.20 Hz, 1H), 6.95 (s, 1H), 6.62-6.76 (m, 1H), 6.52-6.42 (m, 1H), 5.26-5.24 (m, 1H), 4.31 (s, 2H), 3.49-3.15 (m, 8H), 2.91-2.84 (m, 1H), 2.68-2.62 (m, 2H), 2.43-2.33 (m, 2H), 1.99-1.85 (m, 3H), 1.67-1.59 (m, 4H).

### 2-[1-[1-(2,6-dioxo-3-piperidyl)-3-methyl-2-oxo-imidazo[4,5-b]pyridin-5-yl]-4-piperidyl]-N-[5-fluoro-7-hydroxy-6-(1,1,4-trioxo-1,2,5-thiadiazolidin-2-yl)-2-naphthyl]acetamide (Degrader 147)

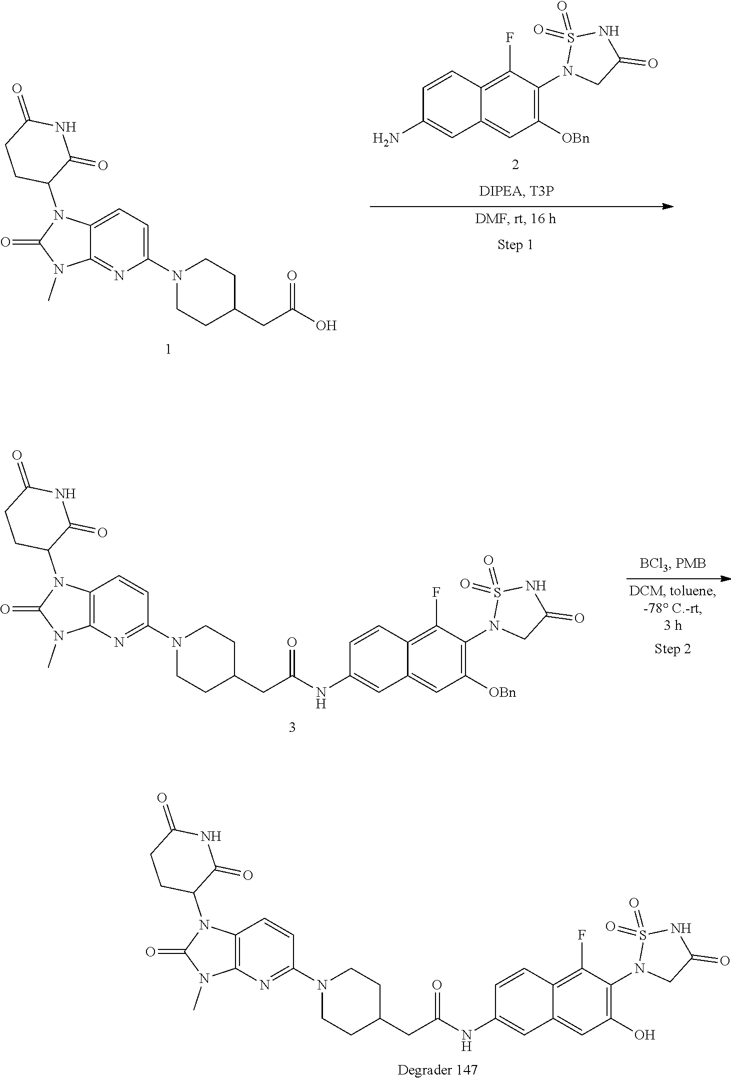 [SMILES: Cn1c(=O)n(C2CCC(=O)NC2=O)c2ccc(N3CCC(CC(=O)O)CC3)nc21 ; Nc1ccc2c(F)c(N3CC(=O)NS3(=O)=O)c(OCc3ccccc3)cc2c1 ; Cn1c(=O)n(C2CCC(=O)NC2=O)c2ccc(N3CCC(CC(=O)Nc4ccc5c(F)c(N6CC(=O)NS6(=O)=O)c(OCc6ccccc6)cc5c4)CC3)nc21 ; Cn1c(=O)n(C2CCC(=O)NC2=O)c2ccc(N3CCC(CC(=O)Nc4ccc5c(F)c(N6CC(=O)NS6(=O)=O)c(O)cc5c4)CC3)nc21]

### Step 1: N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-2-oxo-imidazo[4,5-b]pyridin-5-yl]-4-piperidyl]acetamide (3)

Into a 20 mL screw-capped vial containing a well-stirred solution of 2-[1-[1-(2,6-dioxo-3-piperidyl)-3-methyl-2-oxo-imidazo[4,5-b]pyridin-5-yl]-4-piperidyl]acetic acid (1, 180 mg, 349.22 Mol, TFA salt) in DMF (6 mL) were added propylphosphonic anhydride (50 Wt % in EtOAc) (444.46 mg, 698.43 Mol) and N-ethyl-N-isopropyl-propan-2-amine (225.66 mg, 1.75 mmol, 304.13 μL). After 1 h, 5-(6-amino-3-benzyloxy-1-fluoro-2-naphthyl)-1,1-dioxo-1,2,5-thiadiazolidin-3-one (2, 1.54 mg, 271.96 Mol, TFA salt) was added and stirring was continued at for 16 h. The solvent was evaporated and water was added to precipitate a solid that was filtered and dried to afford N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-2-oxo-imidazo[4,5-b]pyridin-5-yl]-4-piperidyl]acetamide (3, 250 mg, 178.39 μmol, 51% yield, 56% purity) a brown solid.

LCMS (ES+): m/z 785.9 [M+H]⁺

### Step 2: 2-[1-[1-(2,6-dioxo-3-piperidyl)-3-methyl-2-oxo-imidazo[4,5-b]pyridin-5-yl]-4-piperidyl]-N-[5-fluoro-7-hydroxy-6-(1,1,4-trioxo-1,2,5-thiadiazolidin-2-yl)-2-naphthyl]acetamide (Degrader 147)

Into a 25 mL single neck round bottom flask containing well-stirred solution of N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-2-oxo-imidazo[4,5-b]pyridin-5-yl]-4-piperidyl]acetamide (3, 260 mg, 185.52 mol), pentamethylbenzene (110.01 mg, 742.09 μmol) in anhydrous DCM (2 mL) and toluene (2 mL) was added boron trichloride (1.0 M in DCM) (1 M, 1.86 mL) at −78° C. The contents were stirred at room temperature for 3 h. The reaction was quenched with 5 mL of 10% MeOH in DCM at −78° C. Subsequently, the mixture was concentrated under reduced pressure to afford the crude compound, which was purified by reverse phase preparative HPLC [Column: X Select C18 (250×19) mm, 5 microns, Mobile phase: 0.1% FA in water; Mobile phase B: Acetonitrile] to obtain 2-[1-[1-(2,6-dioxo-3-piperidyl)-3-methyl-2-oxo-imidazo[4,5-b]pyridin-5-yl]-4-piperidyl]-N-[5-fluoro-7-hydroxy-6-(1,1,4-trioxo-1,2,5-thiadiazolidin-2-yl)-2-naphthyl]acetamide (Degrader 147, 40 mg, 52.53 μmol, 28% yield, formic acid salt) as an off-white solid.

LCMS (ES−): m/z 693.0 [M−H]⁻

¹H-NMR (400 MHz, DMSO-d6): δ 11.08 (s, 1H), 10.12 (s, 1H), 9.97 (brs, 1H), 8.18 (s, 1H), 7.83 (d, J=9.20 Hz, 1H), 7.42 (dd, J=1.60, 9.00 Hz, 1H), 7.31 (d, J=8.40 Hz, 1H), 6.95 (s, 1H), 6.46 (d, J=8.80 Hz, 1H), 5.32 (dd, J=5.20, 13.00 Hz, 1H), 4.21-4.20 (m, 4H), 3.29 (s, 3H), 2.95-2.75 (m, 3H), 2.71-2.58 (m, 2H), 2.32 (d, J=6.80 Hz, 2H), 2.10-1.99 (m, 2H), 1.81-1.72 (m, 2H), 1.36-1.28 (m, 2H).

### 2-[(3R)-4-[1-(2,6-dioxo-3-piperidyl)-4-fluoro-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 148)

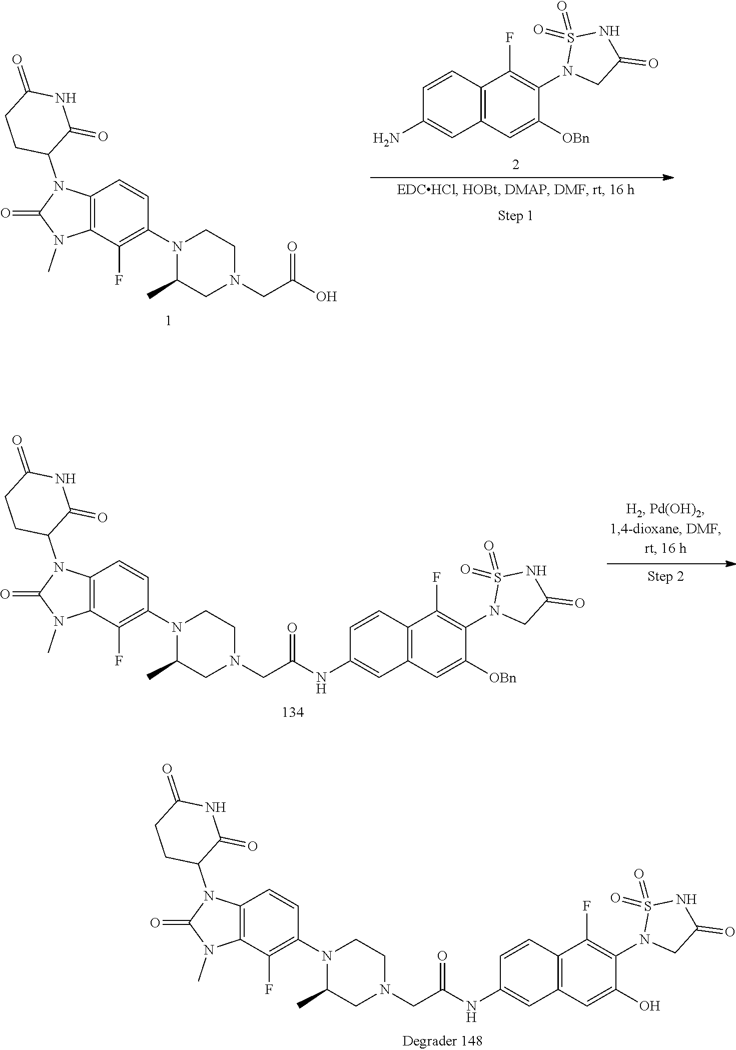 [SMILES: C[C@@H]1CN(CC(=O)O)CCN1c1ccc2c(c1F)n(C)c(=O)n2C1CCC(=O)NC1=O ; Nc1ccc2c(F)c(N3CC(=O)NS3(=O)=O)c(OCc3ccccc3)cc2c1 ; C[C@@H]1CN(CC(=O)Nc2ccc3c(F)c(N4CC(=O)NS4(=O)=O)c(OCc4ccccc4)cc3c2)CCN1c1ccc2c(c1F)n(C)c(=O)n2C1CCC(=O)NC1=O ; C[C@@H]1CN(CC(=O)Nc2ccc3c(F)c(N4CC(=O)NS4(=O)=O)c(O)cc3c2)CCN1c1ccc2c(c1F)n(C)c(=O)n2C1CCC(=O)NC1=O]

2-[(3R)-4-[1-(2,6-dioxo-3-piperidyl)-4-fluoro-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 148, 50 mg, 63.96 μmol, formic acid salt) was synthesized following the same two step procedure as 2-[(3S)-4-[1-(2,6-dioxo-3-piperidyl)-4-fluoro-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 66).

LCMS (ES+): m/z 727.20 [M+H]⁺

¹H-NMR (400 MHz, DMSO-d6): δ 11.13 (s, 1H), 10.86 (bs, 1H), 10.33 (bs, 1H), 9.84 (s, 1H), 8.13 (s, 1H), 7.91 (d, J=8.80 Hz, 1H), 7.49 (dd, J=1.60, 9.20 Hz, 1H), 7.01-6.93 (m, 3H), 5.41-5.37 (m, 1H), 4.30-4.14 (m, 2H), 4.12 (s, 2H), 3.50-3.35 (m, 8H), 3.21-3.12 (m, 1H), 2.95-2.82 (m, 1H), 2.74-2.61 (m, 2H), 2.09-2.03 (m, 1H), 0.90 (s, 3H).

### 2-[3-[1-(2,6-dioxo-3-piperidyl)-3-methyl-2-oxo-benzimidazol-5-yl]pyrazol-1-yl]-N-[5-fluoro-7-hydroxy-6-(1,1,4-trioxo-1,2,5-thiadiazolidin-2-yl)-2-naphthyl]acetamide (Degrader 149)

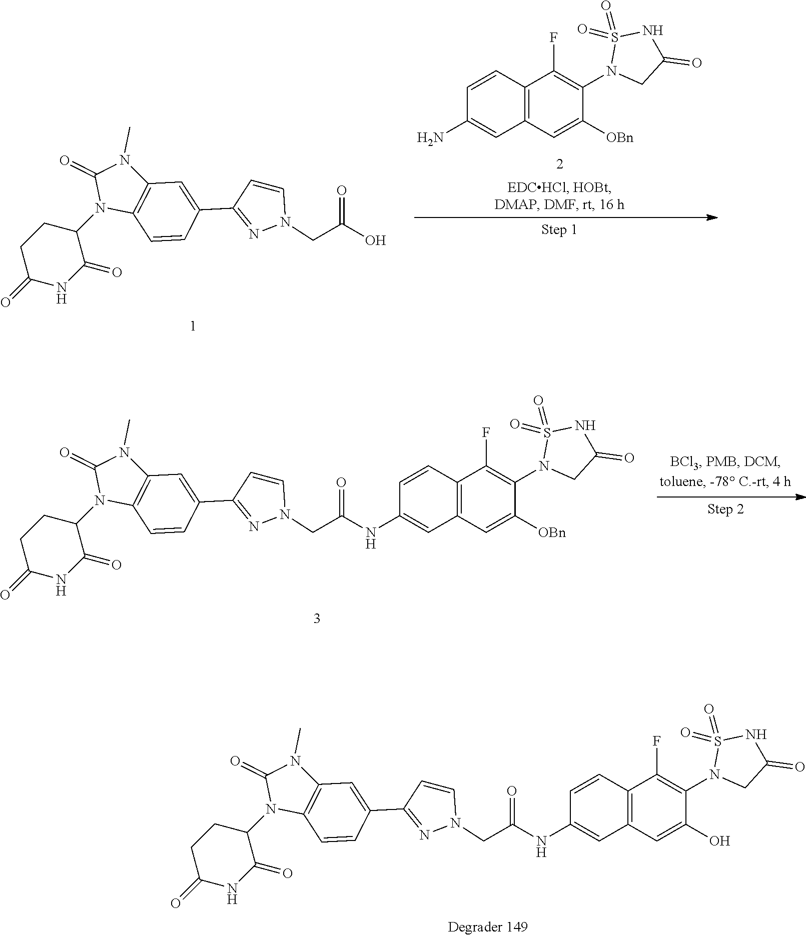 [SMILES: Cn1c(=O)n(C2CCC(=O)NC2=O)c2ccc(-c3ccn(CC(=O)O)n3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(-c3ccn(CC(=O)Nc4ccc5c(F)c(N6CC(=O)NS6(=O)=O)c(OCc6ccccc6)cc5c4)n3)cc21 ; Cn1c(=O)n(C2CCC(=O)NC2=O)c2ccc(-c3ccn(CC(=O)Nc4ccc5c(F)c(N6CC(=O)NS6(=O)=O)c(O)cc5c4)n3)cc21]

### Step 1: N-[7-benzyloxy-5-fluoro-6-(1,1,4-trioxo-1,2,5-thiadiazolidin-2-yl)-2-naphthyl]-2-[3-[1-(2,6-dioxo-3-piperidyl)-3-methyl-2-oxo-benzimidazol-5-yl]pyrazol-1-yl]acetamide (3)

Into a 50 mL single neck round bottom flask containing a well-stirred solution of 2-[3-[1-(2,6-dioxo-3-piperidyl)-3-methyl-2-oxo-benzimidazol-5-yl]pyrazol-1-yl]acetic acid (1, 200 mg, 373.52 Mol, TFA salt) and 5-(6-amino-3-benzyloxy-1-fluoro-2-naphthyl)-1,1-dioxo-1,2,5-thiadiazolidin-3-one (2, 140.93 mg, 280.14 Mol) in anhydrous DMF (5 mL) were added DMAP (273.80 mg, 2.24 mmol), EDC HCl (214.81 mg, 1.12 mmol) followed by HOBt (100.94 mg, 747.03 μmol). After 16 h, the reaction mixture was concentrated under reduced pressure, diluted with cold water (50 mL) and acidified with 1.5N HCl. The solid was filtered, dried and triturated with MTBE (50 mL) to afford N-[7-benzyloxy-5-fluoro-6-(1,1,4-trioxo-1,2,5-thiadiazolidin-2-yl)-2-naphthyl]-2-[3-[1-(2,6-dioxo-3-piperidyl)-3-methyl-2-oxo-benzimidazol-5-yl]pyrazol-1-yl]acetamide (3, 150 mg, 102.98 μmol, 28% yield, 53% purity) as a brown color solid.

LCMS (Es+): m/z 767.0 [M+H]⁺

### Step 2: 2-[3-[1-(2,6-dioxo-3-piperidyl)-3-methyl-2-oxo-benzimidazol-5-yl]pyrazol-1-yl]-N-[5-fluoro-7-hydroxy-6-(1,1,4-trioxo-1,2,5-thiadiazolidin-2-yl)-2-naphthyl]acetamide (Degrader 149)

Into a 50 mL single neck round bottom flask containing well-stirred solution of N-[7-benzyloxy-5-fluoro-6-(1,1,4-trioxo-1,2,5-thiadiazolidin-2-yl)-2-naphthyl]-2-[3-[1-(2,6-dioxo-3-piperidyl)-3-methyl-2-oxo-benzimidazol-5-yl]pyrazol-1-yl]acetamide (3, 160 mg, 109.85 μmol) and pentamethylbenzene (81.42 mg, 549.23 Mol, 88.79 μL) in anhydrous DCM (4 mL) and toluene (4 mL) was added boron trichloride (1.0 M in methylene chloride) (2.20 mmol, 2.20 mL) at −78° C. The mixture was stirred at room temperature for 4 h. The reaction mixture was cooled to −78° C. and was quenched with 5% methanol in DCM (3 mL) and the solvent was removed and the residue washed with MTBE (60 mL) and purified by reverse phase prep HPLC [Purification method: XSelect C18 (250×19) mm, 5 micron; Mobile phase A: 0.1% TFA in water; Mobile phase B: MeCN] to afford 2-[3-[1-(2,6-dioxo-3-piperidyl)-3-methyl-2-oxo-benzimidazol-5-yl]pyrazol-1-yl]-N-[5-fluoro-7-hydroxy-6-(1,1,4-trioxo-1,2,5-thiadiazolidin-2-yl)-2-naphthyl]acetamide (Degrader 149, 34 mg, 38.72 μmol, 35% yield, TFA salt) as an off-white solid.

LCMS (ES+): m/z 677.0 [M+H]⁺

¹H-NMR (400 MHz, DMSO-d6): δ 11.12 (s, 1H), 10.62 (s, 1H), 10.29 (s, 1H), 8.19 (s, 1H), 7.90 (d, J=9.20 Hz, 1H), 7.84 (d, J=2.40 Hz, 1H), 7.60 (d, J=1.60 Hz, 1H), 7.52 (dd, J=1.20, 8.20 Hz, 1H), 7.46 (dd, J=1.60, 9.00 Hz, 1H), 7.15 (d, J=8.00 Hz, 1H), 6.96 (s, 1H), 6.78 (d, J=2.40 Hz, 1H), 5.45-5.35 (m, 1H), 5.14 (s, 2H), 4.32 (s, 2H), 3.39 (s, 3H), 2.99-2.86 (m, 1H), 2.81-2.58 (m, 2H), 2.11-1.99 (m, 1H).

### 2-[(3R)-4-[1-(2,6-dioxo-3-piperidyl)-6-fluoro-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 150)

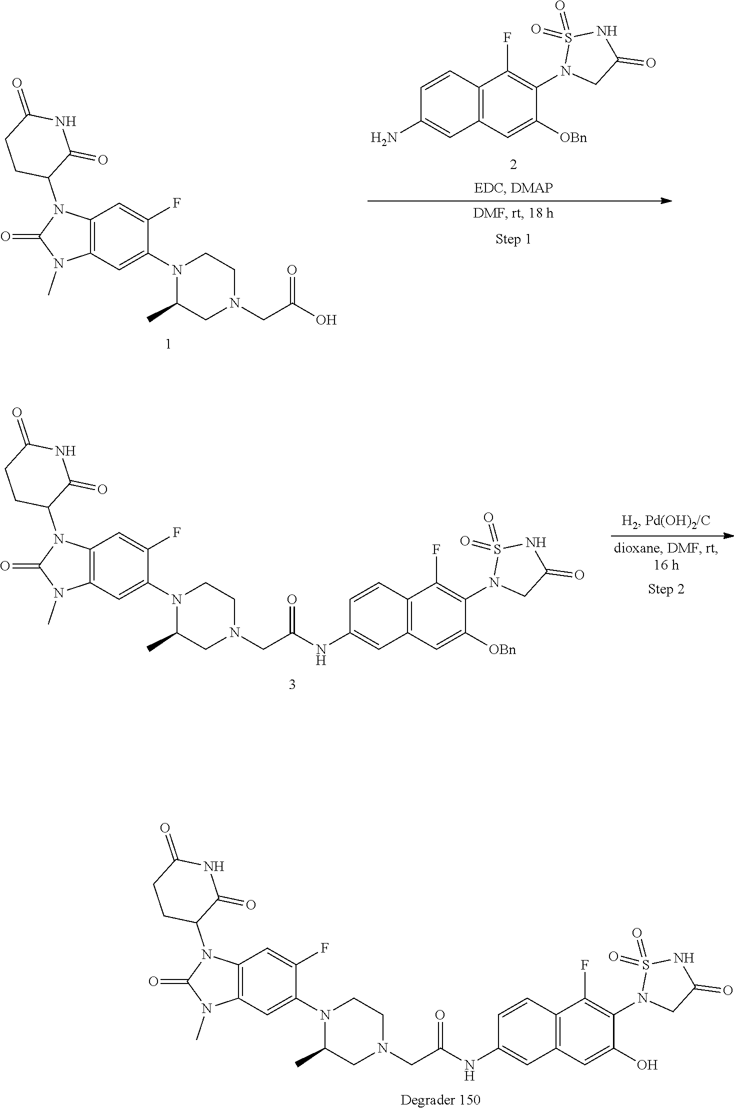 [SMILES: C[C@@H]1CN(CC(=O)O)CCN1c1cc2c(cc1F)n(C1CCC(=O)NC1=O)c(=O)n2C ; Nc1ccc2c(F)c(N3CC(=O)NS3(=O)=O)c(OCc3ccccc3)cc2c1 ; C[C@@H]1CN(CC(=O)Nc2ccc3c(F)c(N4CC(=O)NS4(=O)=O)c(OCc4ccccc4)cc3c2)CCN1c1cc2c(cc1F)n(C1CCC(=O)NC1=O)c(=O)n2C ; C[C@@H]1CN(CC(=O)Nc2ccc3c(F)c(N4CC(=O)NS4(=O)=O)c(O)cc3c2)CCN1c1cc2c(cc1F)n(C1CCC(=O)NC1=O)c(=O)n2C]

### Step 1: N-[7-benzyloxy-5-fluoro-6-(1,1,4-trioxo-1,2,5-thiadiazolidin-2-yl)-2-naphthyl]-2-[(3R)-4-[1-(2,6-dioxo-3-piperidyl)-6-fluoro-3-methyl-2-oxo-benzimidazol-5-yl]-3-methyl-piperazin-1-yl]acetamide (3)

Into a 20 mL screw-capped vial containing a well-stirred solution of 2-[(3R)-4-[1-(2,6-dioxo-3-piperidyl)-6-fluoro-3-methyl-2H-benzimidazol-5-yl]-3-methyl-piperazin-1-yl]acetic acid (1, 100 mg, 224.70 μmol) in DMF (1 mL) were added DMAP (164.71 mg, 1.35 mmol) and 1-ethyl-3-(3-dimethylaminopropyl)carbodiimide hydrochloride (43.07 mg, 224.70 Mol). After 1 h, 5-(6-amino-3-benzyloxy-1-fluoro-2-naphthyl)-1,1-dioxo-1,2,5-thiadiazolidin-3-one (2, 119.14 mg, 224.70 μmol, TFA salt) was added. After 16 h, the solvent was evaporated and water was added to precipitate a solid that was filtered and dried to afford N-[7-benzyloxy-5-fluoro-6-(1,1,4-trioxo-1,2,5-thiadiazolidin-2-yl)-2-naphthyl]-2-[(3R)-4-[1-(2,6-dioxo-3-piperidyl)-6-fluoro-3-methyl-2-oxo-benzimidazol-5-yl]-3-methyl-piperazin-1-yl]acetamide (3, 110 mg, 98.31 μmol, 44% yield, 73% purity) as a brown solid.

UPLC-MS (ES+): m/z 817.9 [M+H]⁺

### Step 2: 2-[(3R)-4-[1-(2,6-dioxo-3-piperidyl)-6-fluoro-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 150)

Into a 25 mL single neck round bottom flask containing a well-stirred solution of N-[7-benzyloxy-5-fluoro-6-(1,1,4-trioxo-1,2,5-thiadiazolidin-2-yl)-2-naphthyl]-2-[(3R)-4-[1-(2,6-dioxo-3-piperidyl)-6-fluoro-3-methyl-2-oxo-benzimidazol-5-yl]-3-methyl-piperazin-1-yl]acetamide (3, 150 mg, 134.06 μmol) in a mixture of DMF (0.6 mL) and dioxane (1.4 mL) was added palladium hydroxide on carbon (20 wt. % loading) (131.79 mg, 938.39 μmol) and the resulting mixture was stirred for 16 h at ambient temperature under hydrogen atmosphere. The reaction mixture was filtered through Celite and washed with a mixture of 1,4-dioxane (10 mL) and DMF (3 mL). The combined filtrate was concentrated, dried and purified by reverse-phase preparative-HPLC [Column: X-BRIDGE C8 (150×19) mm, 5 MICRONS, FLOW: 12 mL, Mobile phase: 0.1% TFA in water; Mobile phase B: Acetonitrile] to obtain 2-[(3R)-4-[1-(2,6-dioxo-3-piperidyl)-6-fluoro-3-methyl-2-oxo-benzimidazol-5-yl]-3-methyl-piperazin-1-yl]-N-[5-fluoro-7-hydroxy-6-(1,1,4-trioxo-1,2,5-thiadiazolidin-2-yl)-2-naphthyl]acetamide (Degrader 150, 22 mg, 23.29 μmol, 17% yield, TFA salt) as an off-white solid.

¹H-NMR (400 MHz, DMSO-d6): δ 11.12 (s, 1H), 10.00 (s, 1H), 8.14 (s, 1H), 7.92 (d, J=8.80 Hz, 1H), 7.49 (d, J=8.80 Hz, 1H), 7.22 (d, J=11.20 Hz, 1H), 7.10 (s, 1H), 7.01 (s, 1H), 5.36 (dd, J=6.00, 12.20 Hz, 1H), 4.35-4.25 (m, 2H), 4.17 (s, 2H), 3.37 (s, 3H), 3.11-3.09 (m, 1H), 2.92-2.85 (m, 1H), 2.79-2.60 (m, 3H), 2.03-2.01 (m, 1H), 0.98 (d, J=7.20 Hz, 3H).

Note: 5 aliphatic protons are obscured by the solvent signal.

LCMS (ES+): m/z 727.0 [M+H]⁺

### 2-[(3R)-3-[1-(2,6-dioxo-3-piperidyl)-3-methyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]acetamide (Degrader 151)

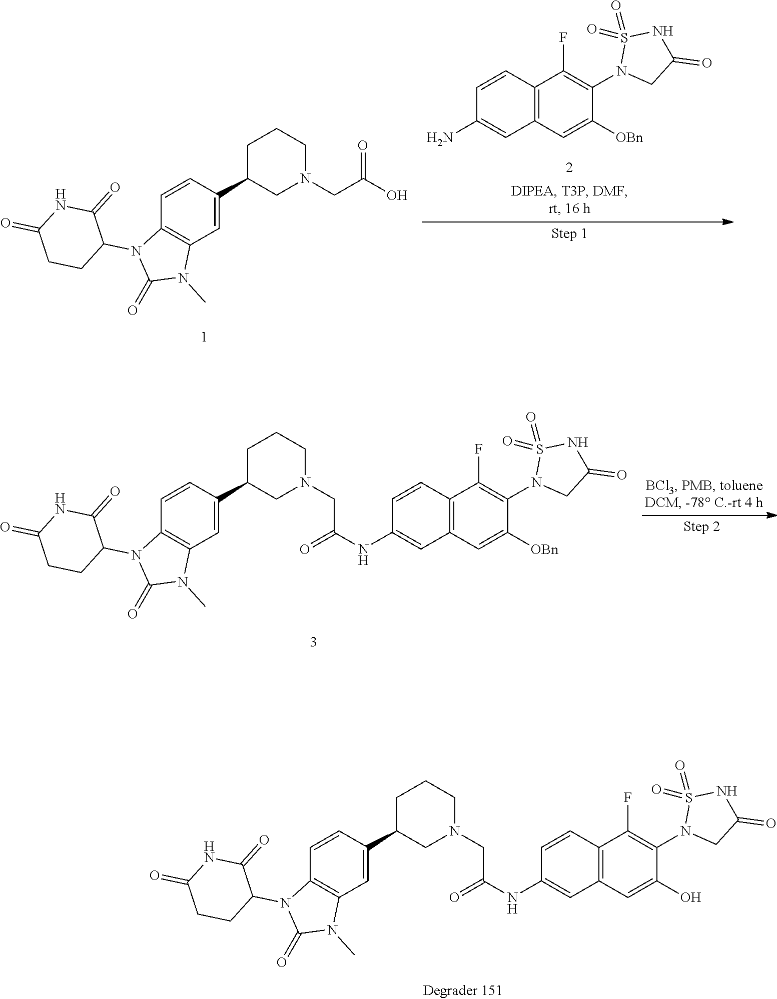 [SMILES: Cn1c(=O)n(C2CCC(=O)NC2=O)c2ccc([C@H]3CCCN(CC(=O)O)C3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc([C@H]3CCCN(CC(=O)Nc4ccc5c(F)c(N6CC(=O)NS6(=O)=O)c(OCc6ccccc6)cc5c4)C3)cc21 ; Cn1c(=O)n(C2CCC(=O)NC2=O)c2ccc([C@H]3CCCN(CC(=O)Nc4ccc5c(F)c(N6CC(=O)NS6(=O)=O)c(O)cc5c4)C3)cc21]

### Step 1: N-[7-benzyloxy-5-fluoro-6-(1,1,4-trioxo-1,2,5-thiadiazolidin-2-yl)-2-naphthyl]-2-[(3R)-3-[1-(2,6-dioxo-3-piperidyl)-3-methyl-2-oxo-benzimidazol-5-yl]-1-piperidyl]acetamide (3)

Into a 50 mL single neck round bottom flask containing a well-stirred solution of 5-(6-amino-3-benzyloxy-1-fluoro-2-naphthyl)-1,1-dioxo-1,2,5-thiadiazolidin-3-one (2, 145.90 mg, 251.92 μmol, TFA salt) and 2-[(3R)-3-[1-(2,6-dioxo-3-piperidyl)-3-methyl-2-oxo-benzimidazol-5-yl]-1-piperidyl]acetic acid (1, 150 mg, 279.91 μmol, TFA salt) in anhydrous DMF (2 mL) was added N,N-diisopropylethylamine (217.05 mg, 1.68 mmol, 292.53 uL), followed by 1-propanephosphonic anhydride (50 wt. % in EtOAc) (302.81 mg, 475.85 μmol). The resulting mixture was stirred at ambient temperature for 16 h. The solvent was removed under reduced pressure and the residue was suspended in aq. 1.5 N HCl (5 mL). The solid was collected by filtration, dried under reduced pressure to obtain N-[7-benzyloxy-5-fluoro-6-(1,1,4-trioxo-1,2,5-thiadiazolidin-2-yl)-2-naphthyl]-2-[(3R)-3-[1-(2,6-dioxo-3-piperidyl)-3-methyl-2-oxo-benzimidazol-5-yl]-1-piperidyl]acetamide (3, 180 mg, 136.05 μmol, 49% yield, 62% purity, HCl salt) as a brown solid.

LCMS (ES+): m/z 784.2 [M+H]⁺

### Step 2: 2-[(3R)-3-[1-(2,6-dioxo-3-piperidyl)-3-methyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]acetamide (Degrader 151)

Into a 50 mL single neck round bottom flask containing a suspension of N-[7-benzyloxy-5-fluoro-6-(1,1,4-trioxo-1,2,5-thiadiazolidin-2-yl)-2-naphthyl]-2-[(3R)-3-[1-(2,6-dioxo-3-piperidyl)-3-methyl-2-oxo-benzimidazol-5-yl]-1-piperidyl]acetamide (3, 180 mg, 136.05 μmol, HCl salt) in a mixture of toluene (4 mL) and DCM (4 mL), was added pentamethylbenzene (100.85 mg, 680.25 μmol). Then boron trichloride (1.0 M in methylene chloride) (4.08 mmol, 4.08 mL) was added at −78° C. The reaction mixture was stirred at ambient temperature for 4 h. The reaction mixture was cooled to −78° C. and quenched with 5% MEOH in DCM (3 mL), concentrated under reduced pressure at 35° C., triturated with MTBE (3×20 mL), filtered and purified by reverse phase prep HPLC [Column: X-select C18 (250×19) mm, 5 micron; Mobile phase A: 0.1% TFA in MQ Water; Mobile phase B: MeCN] to obtain 2-[(3R)-3-[1-(2,6-dioxo-3-piperidyl)-3-methyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]acetamide (Degrader 151, 52 mg, 63.21 μmol, 46% yield, TFA salt) as an off-white solid.

LCMS (ES−): m/z 692.4 [M−H]⁻

¹H-NMR (400 MHz, DMSO-d6): δ 11.10 (s, 1H), 10.77 (s, 1H), 9.96 (bs, 2H), 8.11 (s, 1H), 7.90 (d, J=8.80 Hz, 1H), 7.45 (d, J=9.20 Hz, 1H), 7.14-7.10 (m, 2H), 7.00-6.96 (m, 2H), 5.39-5.35 (m, 1H), 4.22 (s, 2H), 4.16 (s, 2H), 3.67-3.58 (m, 2H), 3.36 (s, 3H), 3.29-3.16 (m, 3H), 2.92-2.87 (m, 1H), 2.88-2.61 (m, 2H), 2.05-1.89 (m, 4H), 1.81-1.68 (m, 1H).

### 2-[(3S)-3-[1-(2,6-dioxo-3-piperidyl)-3-methyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]acetamide (Degrader 152)

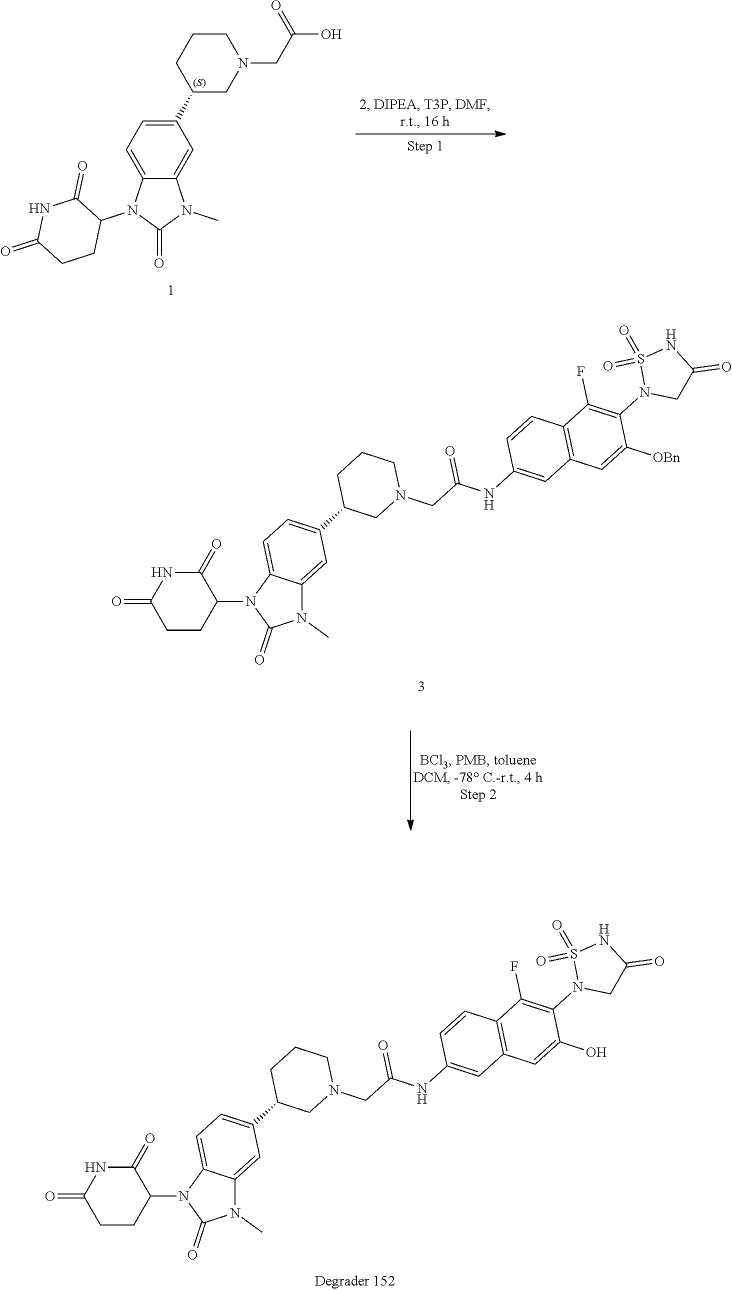

2-[(3S)-3-[1-(2,6-dioxo-3-piperidyl)-3-methyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]acetamide (Degrader 152, 50 mg, 60.01 μmol, TFA salt) was synthesized in two steps following the same procedure as 2-[(3R)-3-[1-(2,6-dioxo-3-piperidyl)-3-methyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]acetamide (Degrader 151).

LCMS (ES−): m/z 692 [M−H]⁻

¹H-NMR (400 MHz, DMSO-d6): δ 11.10 (s, 1H), 10.77 (s, 1H), 9.99-9.95 (broad m, 2H), 8.11 (s, 1H), 7.90 (d, J=8.80 Hz, 1H), 7.45 (d, J=9.20 Hz, 1H), 7.14-7.10 (m, 2H), 7.00-6.96 (m, 2H), 5.39-5.35 (m, 1H), 4.22 (s, 2H), 4.16 (s, 2H), 3.64-3.55 (m, 2H), 3.36 (s, 3H), 3.26-3.09 (m, 3H), 2.94-2.87 (m, 1H), 2.76-2.57 (m, 2H), 2.01-1.93 (m, 4H), 1.81-1.65 (m, 1H).

### 2-[1-[1-(2,6-dioxo-3-piperidyl)-3-methyl-2-oxo-benzimidazol-5-yl]pyrazol-4-yl]-N-[5-fluoro-7-hydroxy-6-(1,1,4-trioxo-1,2,5-thiadiazolidin-2-yl)-2-naphthyl]acetamide (Degrader 153)

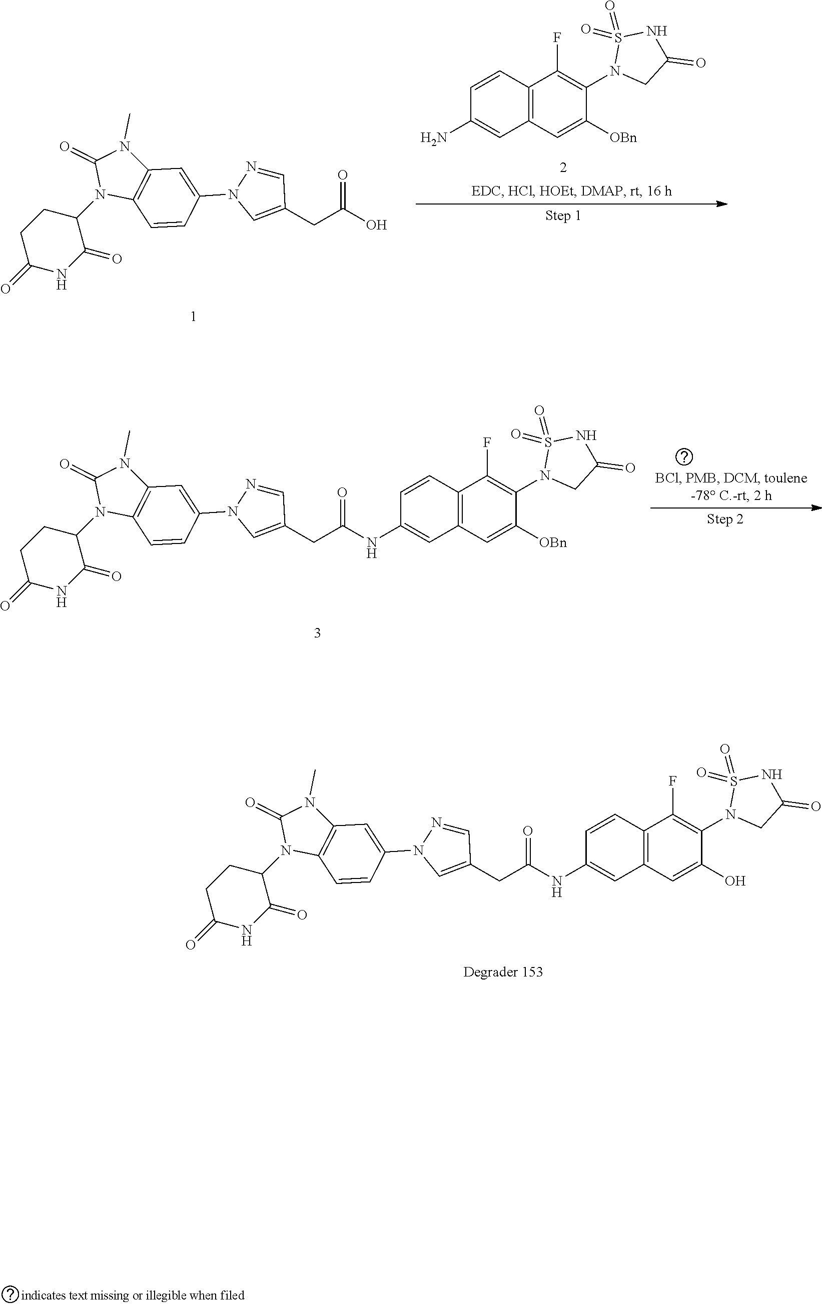 [SMILES: Cn1c(=O)n(C2CCC(=O)NC2=O)c2ccc(-n3cc(CC(=O)O)cn3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(-n3cc(CC(=O)Nc4ccc5c(F)c(N6CC(=O)NS6(=O)=O)c(OCc6ccccc6)cc5c4)cn3)cc21 ; Cn1c(=O)n(C2CCC(=O)NC2=O)c2ccc(-n3cc(CC(=O)Nc4ccc5c(F)c(N6CC(=O)NS6(=O)=O)c(O)cc5c4)cn3)cc21]

### Step 1: N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-2-oxo-benzimidazol-5-yl]pyrazol-4-yl]acetamide (3)

To a 25 mL single neck round bottom flask containing 2-[1-[1-(2,6-dioxo-3-piperidyl)-3-methyl-2-oxo-benzimidazol-5-yl]pyrazol-4-yl]acetic acid (1, 100 mg, 253.03 Mol), 5-(6-amino-3-benzyloxy-1-fluoro-2-naphthyl)-1,1-dioxo-1,2,5-thiadiazolidin-3-one (2, 134.45 mg, 253.03 μmol, TFA salt), EDC HCl (242.60 mg, 1.27 mmol), HOBt (102.57 mg, 759.08 Mol) and DMAP (309.12 mg, 2.53 mmol) was added DMF (3 mL) and the resulting solution was stirred at room temperature for 16 h. The reaction mixture was diluted with 1.5N HCl (20 mL) and the precipitate filtered and purified by reverse phase column chromatography [Silicycle C18 column; Mobile phase A: 0.1% Formic acid in water; Mobile phase B: MeCN] to afford N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-2-oxo-benzimidazol-5-yl]pyrazol-4-yl]acetamide (3, 75 mg, 82.28 μmol, 33% yield, formic acid salt) as an off-white solid.

LCMS (ES+): m/z 767.0 [M+H]⁺

### Step 2: 2-[1-[1-(2,6-dioxo-3-piperidyl)-3-methyl-2-oxo-benzimidazol-5-yl]pyrazol-4-yl]-N-[5-fluoro-7-hydroxy-6-(1,1,4-trioxo-1,2,5-thiadiazolidin-2-yl)-2-naphthyl]acetamide (Degrader 153)

Into a 25 mL single neck round bottom flask containing well-stirred solution of N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-2-oxo-benzimidazol-5-yl]pyrazol-4-yl]acetamide (3, 75 mg, 82.28 μmol, formic acid salt) and pentamethylbenzene (73.19 mg, 493.70 μmol, 79.81 μL) in DCM (1.5 mL) and toluene (1.5 mL) was added boron trichloride (1 M in DCM) (152.32 mg, 1.3 mmol, 1.3 mL) at −78° C. The reaction mixture was stirred at room temperature for 2 h. The reaction mixture was cooled to −78° C. and was quenched with 5% methanol in DCM (2 mL) and the solvent was removed and the residue was washed with MTBE (40 mL) and purified by reverse phase column chromatography [Silicycle C18 column; Mobile phase A: 0.1% Formic acid in water; Mobile phase B: MeCN] to afford 2-[1-[1-(2,6-dioxo-3-piperidyl)-3-methyl-2-oxo-benzimidazol-5-yl]pyrazol-4-yl]-N-[5-fluoro-7-hydroxy-6-(1,1,4-trioxo-1,2,5-thiadiazolidin-2-yl)-2-naphthyl]acetamide (Degrader 153, 35 mg, 49.81 μmol, 61% yield) as off white solid.

LCMS (ES−): m/z 675.0 [M−H]⁻

¹HNMR (400 MHz, DMSO-d6): δ 11.13 (s, 1H), 10.36 (s, 1H), 9.75 (s, 1H), 8.40 (s, 1H), 8.17 (s, 1H), 7.84 (d, J=8.80 Hz, 1H), 7.70-7.68 (m, 2H), 7.51 (dd, J=2.00, 8.40 Hz, 1H), 7.46 (dd, J=1.60, 9.20 Hz, 1H), 7.23 (d, J=8.40 Hz, 1H), 6.94 (s, 1H), 5.43-5.39 (m, 1H), 4.07 (s, 2H), 3.67 (s, 2H), 3.37 (s, 3H), 2.95-2.87 (m, 1H), 2.79-2.72 (m, 1H), 2.68-2.67 (m, 1H), 2.12-2.01 (m, 1H).

### 3-[5-[1-[2-[4-[8-fluoro-6-hydroxy-7-(1,1,4-trioxo-1,2,5-thiadiazolidin-2-yl)-2-naphthyl]-1-piperidyl]-2-oxo-ethyl]-4-piperidyl]indolin-1-yl]piperidine-2,6-dione (Degrader 154)

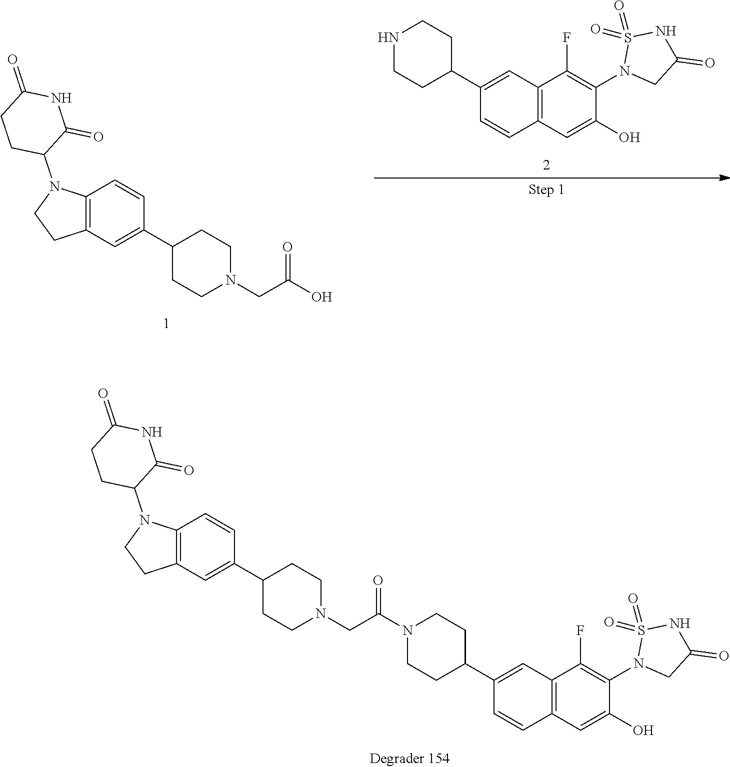 [SMILES: O=C(O)CN1CCC(c2ccc3c(c2)CCN3C2CCC(=O)NC2=O)CC1 ; O=C1CN(c2c(O)cc3ccc(C4CCNCC4)cc3c2F)S(=O)(=O)N1 ; O=C1CCC(N2CCc3cc(C4CCN(CC(=O)N5CCC(c6ccc7cc(O)c(N8CC(=O)NS8(=O)=O)c(F)c7c6)CC5)CC4)ccc32)C(=O)N1]

### Step 1: 3-[5-[1-[2-[4-[8-fluoro-6-hydroxy-7-(1,1,4-trioxo-1,2,5-thiadiazolidin-2-yl)-2-naphthyl]-1-piperidyl]-2-oxo-ethyl]-4-piperidyl] indolin-1-yl]piperidine-2,6-dione (Degrader 154)

Into a 20 mL vial containing a well-stirred solution of 2-[4-[1-(2,6-dioxo-3-piperidyl) indolin-5-yl]-1-piperidyl]acetic acid (1, 70 mg, 102.38 μmol, TFA salt) in DMF (2 mL) was added DIPEA (66.16 mg, 511.89 μmol, 89.16 μL) and propylphosphonic anhydride (50 wt. % in ethyl acetate) (97.72 mg, 153.57 μmol). The reaction mixture was stirred for 5 min and 5-[1-fluoro-3-hydroxy-7-(4-piperidyl)-2-naphthyl]-1,1-dioxo-1,2,5-thiadiazolidin-3-one (2, 41.77 mg, 78.72 μmol, TFA salt) was added. Then reaction mixture was stirred at ambient temperature for 16 h. The reaction mixture was concentrated and purified by reverse phase column chromatography [Silicycle C18 column; Mobile phase A: 0.1% Formic acid in water; Mobile phase B: MeCN] to afford 3-[5-[1-[2-[4-[8-fluoro-6-hydroxy-7-(1,1,4-trioxo-1,2,5-thiadiazolidin-2-yl)-2-naphthyl]-1-piperidyl]-2-oxo-ethyl]-4-piperidyl] indolin-1-yl]piperidine-2,6-dione (Degrader 154, 19 mg, 21.47 μmol, 21% yield, formic acid salt) as an off white solid.

LCMS (ES+): m/z 733.2 [M+H]⁺

¹HNMR (400 MHz, DMSO-d6): δ 10.79 (s, 1H), 9.71 (s, 1H), 9.44 (bs, 1H), 7.72-7.67 (m, 2H), 7.41 (dd, J=1.60, 8.60 Hz, 1H), 7.05 (s, 1H), 6.93 (s, 1H), 6.84 (d, J=6.00 Hz, 1H), 6.44 (d, J=8.00 Hz, 1H), 4.63-4.58 (m, 2H), 4.50-4.21 (m, 2H), 4.09 (s, 2H), 3.89-3.51 (broad m, 6H), 3.31-3.15 (m, 4H), 3.05-2.62 (m, 4H), 2.67-2.60 (m, 2H), 2.22-2.15 (m, 1H), 1.94-1.76 (m, 7H), 1.62-1.51 (m, 1H).

### 2-[4-[1-(2,6-dioxo-3-piperidyl)-2-oxo-benzo[cd]indol-6-yl]phenyl]-N-[5-fluoro-7-hydroxy-6-(1,1,4-trioxo-1,2,5-thiadiazolidin-2-yl)-2-naphthyl]acetamide (Degrader 155)

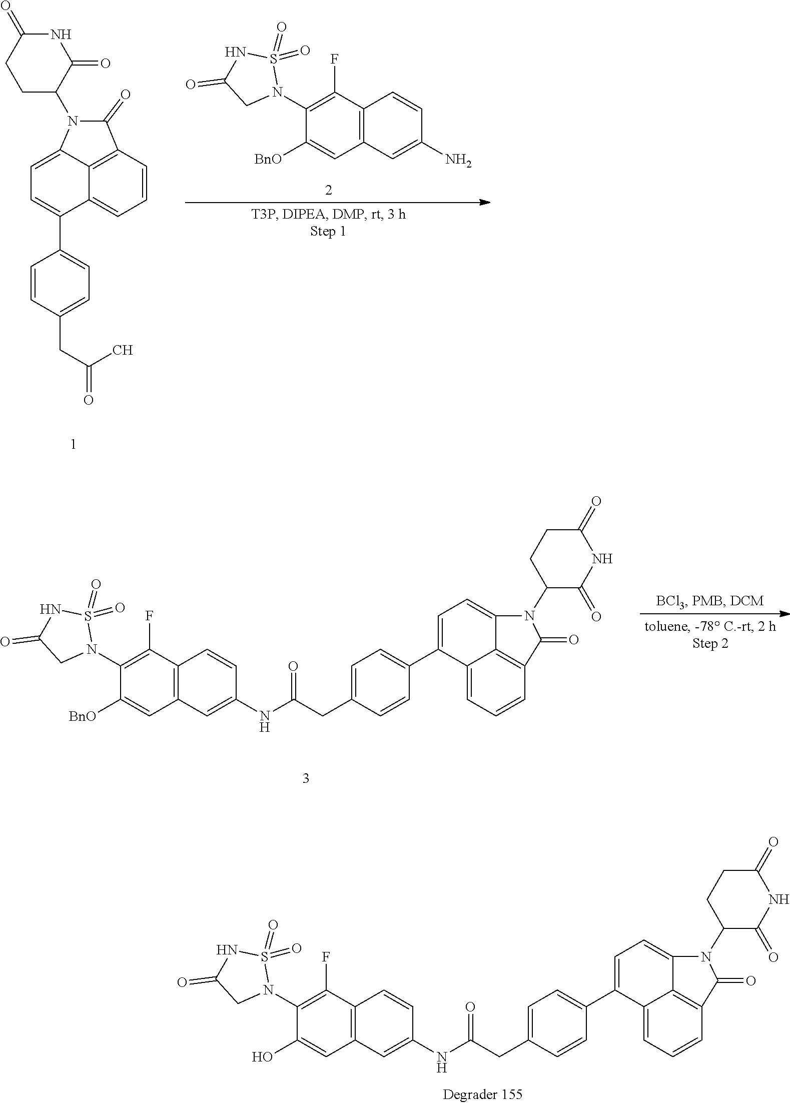 [SMILES: CC(=O)Cc1ccc(-c2ccc3c4c(cccc24)C(=O)N3C2CCC(=O)NC2=O)cc1 ; Nc1ccc2c(F)c(N3CC(=O)NS3(=O)=O)c(OCc3ccccc3)cc2c1 ; O=C1CCC(N2C(=O)c3cccc4c(-c5ccc(CC(=O)Nc6ccc7c(F)c(N8CC(=O)NS8(=O)=O)c(OCc8ccccc8)cc7c6)cc5)ccc2c34)C(=O)N1 ; O=C1CCC(N2C(=O)c3cccc4c(-c5ccc(CC(=O)Nc6ccc7c(F)c(N8CC(=O)NS8(=O)=O)c(O)cc7c6)cc5)ccc2c34)C(=O)N1]

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-2-oxo-benzo[cd]indol-6-yl]phenyl]acetamide (3)

To a stirred solution of 2-[4-[1-(2,6-dioxo-3-piperidyl)-2-oxo-benzo[cd]indol-6-yl]phenyl]acetic acid (1, 210.00 mg, 354.73 μmol) in anhydrous DMF (5 mL) were added DIPEA (91.69 mg, 709.46 μmol, 123.58 μL), T₃P (50 wt. % in EtOAc) (225.61 mg, 354.73 μmol) and 5-(6-amino-3-benzyloxy-1-fluoro-2-naphthyl)-1,1-dioxo-1,2,5-thiadiazolidin-3-one (2, 200 mg, 354.73 mol, TFA salt) at 0° C. under nitrogen atmosphere. The resulting reaction mixture was stirred at room temperature for 3 h. The reaction mixture was treated with ice water (3 mL) and the precipitate filtered and dried under vacuum to afford N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-2-oxo-benzo[cd]indol-6-yl]phenyl]acetamide (3, 450 mg, 327.15 μmol, 92% yield, 58% purity) as a gummy solid.

UPLC-MS (ES+): m/z 798.1 [M+H]⁺

### Step 2: 2-[4-[1-(2,6-dioxo-3-piperidyl)-2-oxo-benzo[cd]indol-6-yl]phenyl]-N-[5-fluoro-7-hydroxy-6-(1,1,4-trioxo-1,2,5-thiadiazolidin-2-yl)-2-naphthyl]acetamide (Degrader 155)

Into a 100 mL single neck round bottom flask containing a well-stirred solution of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-2-oxo-benzo[cd]indol-6-yl]phenyl]acetamide (3, 450 mg, 327.15 μmol) and pentamethylbenzene (387.98 mg, 2.62 mmol) in anhydrous DCM (4 mL) and toluene (1 mL) was added BCl₃ (1.0 M in DCM) (6.45 mmol, 6.54 mL) at −78° C. The reaction mixture was stirred at room temperature for 3 h. Reaction mixture was quenched with 5 mL of 5% MeOH in DCM at −78° C. and excess solvents were removed and the residue purified by reverse phase prep HPLC [Purification method: Column X Bridge C18 (150×19 mm) 5 microns; Mobile phase A: 0.1% Formic acid in water; Mobile phase B: MeCN] to obtain 2-[4-[1-(2,6-dioxo-3-piperidyl)-2-oxo-benzo[cd]indol-6-yl]phenyl]-N-[5-fluoro-7-hydroxy-6-(1,1,4-trioxo-1,2,5-thiadiazolidin-2-yl)-2-naphthyl]acetamide (Degrader 155, 12 mg, 15.21 μmol, 5% yield) as a yellow solid.

LCMS (ES+): m/z 706.0 [M+H]⁺

¹H-NMR (400 MHz, DMSO-d6): δ 11.16 (s, 1H), 10.51 (s, 1H), 10.42 (brs, 1H), 8.22-8.20 (m, 2H), 8.17 (d, J=7.20 Hz, 1H), 7.90-7.86 (m, 2H), 7.57-7.49 (m, 6H), 7.27 (d, J=7.60 Hz, 1H), 6.98 (s, 1H), 5.51 (dd, J=4.80, 12.80 Hz, 1H), 4.40 (s, 2H), 3.82 (s, 2H), 3.15-2.90 (m, 1H), 2.89-2.72 (m, 1H), 2.70-2.63 (m, 1H), 2.15-2.08 (m, 1H).

### 2-[1-[1-(2,6-dioxo-3-piperidyl)-3-methyl-2-oxo-benzimidazol-5-yl]-3,5-dimethyl-pyrazol-4-yl]-N-[5-fluoro-7-hydroxy-6-(1,1,4-trioxo-1,2,5-thiadiazolidin-2-yl)-2-naphthyl]acetamide (Degrader 156)

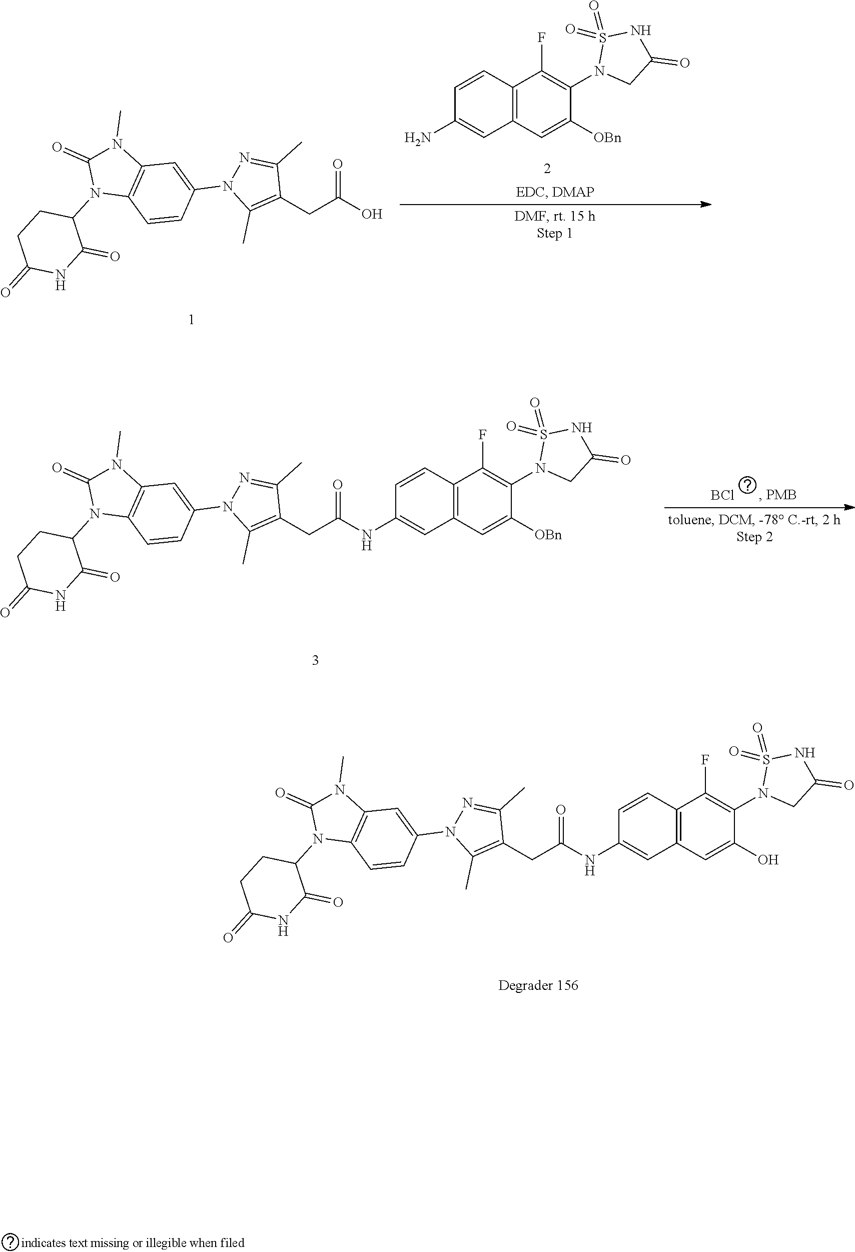 [SMILES: Cc1nn(-c2ccc3c(c2)n(C)c(=O)n3C2CCC(=O)NC2=O)c(C)c1CC(=O)O ; Nc1ccc2c(F)c(N3CC(=O)NS3(=O)=O)c(OCc3ccccc3)cc2c1 ; Cc1nn(-c2ccc3c(c2)n(C)c(=O)n3C2CCC(=O)NC2=O)c(C)c1CC(=O)Nc1ccc2c(F)c(N3CC(=O)NS3(=O)=O)c(OCc3ccccc3)cc2c1 ; "BCl" ; Cc1nn(-c2ccc3c(c2)n(C)c(=O)n3C2CCC(=O)NC2=O)c(C)c1CC(=O)Nc1ccc2c(F)c(N3CC(=O)NS3(=O)=O)c(O)cc2c1]

### Step 1: N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-2-oxo-benzimidazol-5-yl]-3,5-dimethyl-pyrazol-4-yl]acetamide (3)

Into a 20 mL screw-capped vial containing a well-stirred solution of 2-[1-[1-(2,6-dioxo-3-piperidyl)-3-methyl-2-oxo-benzimidazol-5-yl]-3,5-dimethyl-pyrazol-4-yl]acetic acid (1, 200 mg, 437.52 μmol) in anhydrous DMF (5 mL) were added DMAP (267.26 mg, 2.19 mmol), EDC HCl (251.62 mg, 1.31 mmol). After 1 h, 5-(6-amino-3-benzyloxy-1-fluoro-2-naphthyl)-1,1-dioxo-1,2,5-thiadiazolidin-3-one (2, 247.82 mg, 437.52 μmol, TFA salt) was added. After 15 h, the mixture was concentrated and treated with ice-cold water (20 mL) to give a precipitate that was filtered and dried to afford N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-2-oxo-benzimidazol-5-yl]-3,5-dimethyl-pyrazol-4-yl]acetamide (3, 200 mg, 191.74 μmol, 44% yield, 76% purity) as an off-white solid.

LCMS (ES+): m/z 795.7 [M+H]⁺

### Step 2: 2-[1-[1-(2,6-dioxo-3-piperidyl)-3-methyl-2-oxo-benzimidazol-5-yl]-3,5-dimethyl-pyrazol-4-yl]-N-[5-fluoro-7-hydroxy-6-(1,1,4-trioxo-1,2,5-thiadiazolidin-2-yl)-2-naphthyl]acetamide (Degrader 156)

Into a 50 mL single-neck round bottom flask containing a well-stirred solution of N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-2-oxo-benzimidazol-5-yl]-3,5-dimethyl-pyrazol-4-yl]acetamide (3, 200 mg, 191.24 μmol) and pentamethylbenzene (141.75 mg, 956.21 μmol) in anhydrous toluene (5 mL) and DCM (5 mL) was added boron trichloride solution (1.0 M in DCM) (3.82 mmol, 3.82 mL) at −78° C. The reaction mixture was stirred at ambient temperature for 2 h. The reaction was quenched with 5 mL of 5% MeOH in DCM at −78° C., concentrated and purified by reverse-phase preparative HPLC [Column: X-SELECT C18 (250×19) MM, 5 MICRONS, Mobile phase A: 0.1% FA in water and Mobile Phase B: MeCN] to afford 2-[1-[1-(2,6-dioxo-3-piperidyl)-3-methyl-2-oxo-benzimidazol-5-yl]-3,5-dimethyl-pyrazol-4-yl]-N-[5-fluoro-7-hydroxy-6-(1,1,4-trioxo-1,2,5-thiadiazolidin-2-yl)-2-naphthyl]acetamide (Degrader 156, 67 mg, 92.67 μmol, 48% yield) as a white solid.

LCMS (ES−): m/z 703.0 [M−H]⁻

¹HNMR (400 MHz, DMSO-d6): δ 11.14 (s, 1H), 10.45 (s, 1H), 10.34 (s, 1H), 8.20 (s, 1H), 7.88 (d, J=8.80 Hz, 1H), 7.48 (dd, J=1.20, 9.00 Hz, 1H), 7.34 (d, J=2.00 Hz, 1H), 7.22 (d, J=8.40 Hz, 1H), 7.13 (d, J=8.40 Hz, 1H), 6.97 (s, 1H), 5.46-5.41 (m, 1H), 4.41 (s, 2H), 3.56 (s, 3H), 3.38 (s, 3H), 2.95-2.88 (m, 1H), 2.78-2.73 (m, 1H), 2.29 (s, 3H), 2.22 (s, 3H), 2.16-2.09 (m, 1H).

### 2-[4-[1-(2,6-dioxo-3-piperidyl)-3-methyl-2-oxo-benzimidazol-5-yl]cyclohex-3-en-1-yl]-N-[5-fluoro-7-hydroxy-6-(1,1,4-trioxo-1,2,5-thiadiazolidin-2-yl)-2-naphthyl]acetamide (Degrader 157)

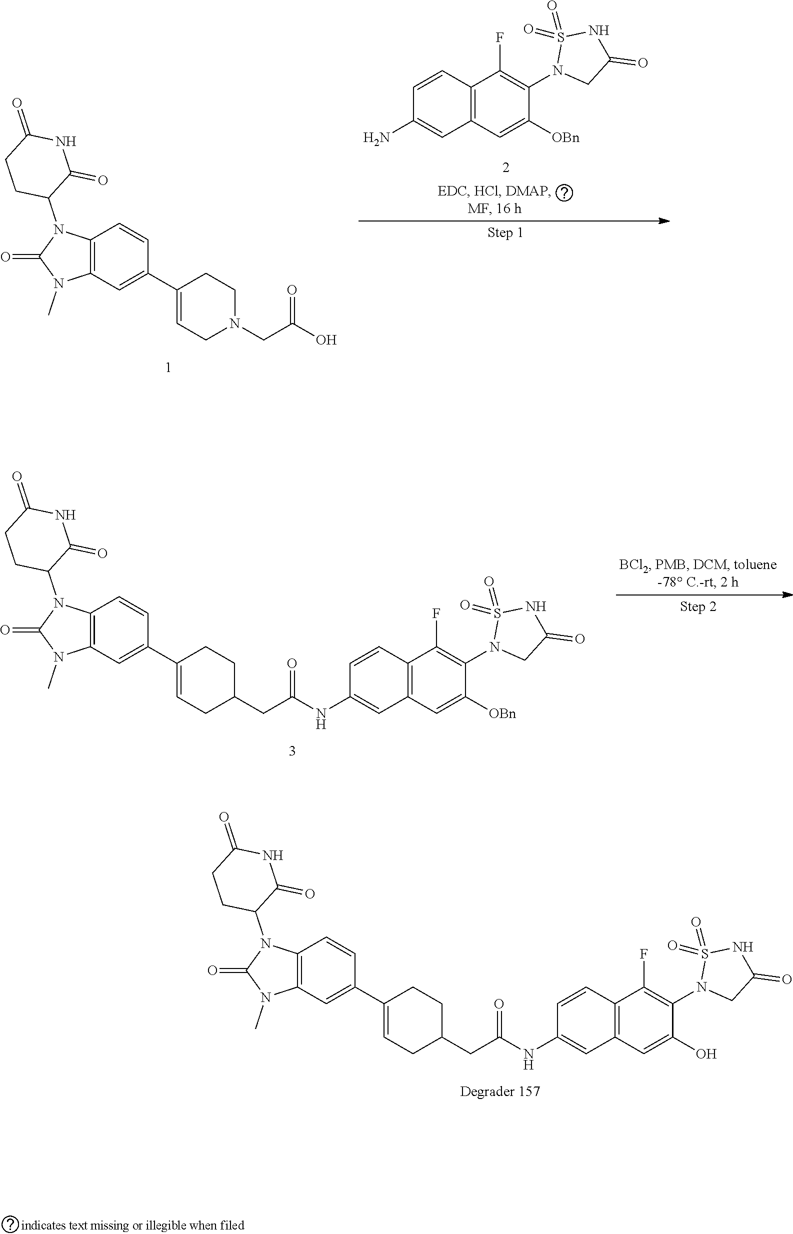 [SMILES: Cn1c(=O)n(C2CCC(=O)NC2=O)c2ccc(C3=CCN(CC(=O)O)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(C3=CCC(CC(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(C3=CCC(CC(=O)Nc4ccc5c(F)c(N6CC(=O)NS6(=O)=O)c(O)cc5c4)CC3)cc21]

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]cyclohex-3-en-1-yl]acetamide (3)

Into a 20 mL vial containing a well-stirred solution of 2-[4-[1-(2,6-dioxo-3-piperidyl)-3-methyl-2-oxo-benzimidazol-5-yl] cyclohex-3-en-1-yl]acetic acid (1, 45 mg, 97.38 Mol) in DMF (2 mL) were added 5-(6-amino-3-benzyloxy-1-fluoro-2-naphthyl)-1,1-dioxo-1,2,5-thiadiazolidin-3-one (2, 40.30 mg, 75.84 Mol, TFA salt), EDC HCl (56.00 mg, 292.13 Mol) and DMAP (59.48 mg, 486.89 μmol). The reaction mixture was heated at 60° C. for 16 h. After completion of the reaction, the solvent was removed under reduced pressure and the residue was suspended in 1.5N HCl (1 mL) was added. The precipitate was filtered, washed with water and dried under vacuum to afford N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]cyclohex-3-en-1-yl]acetamide (3, 95 mg, 66.82 μmol, 69% yield, 55% purity) crude compound as an off white solid.

LCMS (ES+): m/z 781.0 [M+H]⁺

### Step 2: 2-[4-[1-(2,6-dioxo-3-piperidyl)-3-methyl-2-oxo-benzimidazol-5-yl]cyclohex-3-en-1-yl]-N-[5-fluoro-7-hydroxy-6-(1,1,4-trioxo-1,2,5-thiadiazolidin-2-yl)-2-naphthyl]acetamide (Degrader 157)

Into a 50 mL single neck round bottom flask containing a well-stirred solution of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]cyclohex-3-en-1-yl]acetamide (3, 95 mg, 68.13 mol) in anhydrous toluene (1.5 mL) and DCM (1.5 mL) was added pentamethylbenzene (50.50 mg, 340.67 μmol). The reaction mixture was cooled to −78° C. and boron trichloride (1.0 M in DCM) (1.36 mmol, 1.36 mL) was added. The reaction mixture was stirred at room temperature for 2 h. The reaction mixture was cooled to −78° C. and quenched slowly with 5% methanol in dichloromethane (1 mL). The solvent was removed under reduced pressure and the residue was washed with diethyl ether (10 mL), filtered, dried and purified by reverse phase column chromatography [Silicycle C18 column; Mobile phase A: 0.1% Formic acid in water; Mobile phase B: MeCN] to afford 2-[4-[1-(2,6-dioxo-3-piperidyl)-3-methyl-2-oxo-benzimidazol-5-yl]cyclohex-3-en-1-yl]-N-[5-fluoro-7-hydroxy-6-(1,1,4-trioxo-1,2,5-thiadiazolidin-2-yl)-2-naphthyl]acetamide (Degrader 157, 13 mg, 16.32 μmol, 24% yield) as an off-white solid.

LCMS (ES+): m/z 691.2 [M+H]⁺

¹HNMR (400 MHz, DMSO-d6): δ 11.09 (s, 1H), 10.15 (s, 1H), 9.70 (s, 1H), 8.18 (s, 1H), 7.82 (d, J=8.80 Hz, 1H), 7.44 (dd, J=1.60, 9.20 Hz, 1H), 7.25 (s, 1H), 7.11 (dd, J=1.20, 8.40 Hz, 1H), 7.05 (d, J=8.00 Hz, 1H), 6.95 (s, 1H), 6.12 (s, 1H), 5.38-5.33 (m, 1H), 4.07 (s, 2H), 2.94-2.86 (m, 2H), 2.75-2.61 (m, 3H), 2.41-2.34 (m, 3H), 2.21-2.13 (m, 1H), 2.08-1.94 (m, 4H), 1.52-1.42 (m, 1H).

### HiBit Method

### Materials

Dulbecco's modified Eagle medium (DMEM) without phenol red and fetal bovine serum (FBS) were purchased from Gibco (Grand Island, NY, USA). Nano-Glo® HiBiT Lytic Assay System was purchased from Promega (Medison, WI, USA). 293T. 109 (HiBiT-PTPN2) cell line was generated by ectopically expressing PTPN2 with N-terminal HiBiT fusion tag in 293T WT cell line purchased from ATCC (Manassas, VA, USA). Cell culture flasks and 384-well microplates were acquired from VWR (Radnor, PA, USA).

### PTPN2 Degradation Analysis

PTPN2 degradation was determined based on quantification of luminescent signal using Nano-Glo® HiBiT Lytic Assay kit. Test compounds were added to the 384-well plate from a top concentration of 10 μM with 11 points, half log titration in duplicates. 293T. 109 cells were added into 384-well plates at a cell density of 5000 cells per well. The plates were kept at 37° C. with 5% CO₂ for 24 hours. The cells treated in the absence of the test compound were the negative control and the cells without Nano-Glo® HiBiT Lytic reagent were the positive control. After 24-hour incubation, Nano-Glo® HiBiT Lytic Assay reagents were added to the cells. Luminescence was acquired on EnVision™ Multilabel Reader (PerkinElmer, Santa Clara, CA, USA).

TABLE 2

| | HiBiT- | | HiBiT- | |
|---|---|---|---|---|
| | Degradation | | Degradation | |
| | 293T.109 PTPN2 | | B16F10.4 PTPN2 | |
| Degrader. | 24.0 hours | | 24.0 hours | |
| No. | DC₅₀ [nM] | Eₘₐₓ | DC₅₀ [nM] | Eₘₐₓ |
| 1 | A | A | A | A |
| 2 | ND | ND | ND | ND |
| 3 | ND | ND | ND | ND |
| 4 | ND | ND | ND | ND |
| 5 | ND | ND | ND | ND |
| 6 | A | A | B | A |
| 7 | A | A | C | B |
| 8 | B | A | B | A |
| 9 | C | A | C | A |
| 10 | B | B | C | C |
| 11 | B | B | C | B |
| 12 | A | A | B | A |
| 13 | B | B | B | B |
| 14 | B | A | C | B |
| 15 | ND | ND | ND | ND |
| 16 | C | A | D | B |
| 17 | B | B | C | B |
| 18 | B | B | C | B |
| 19 | A | A | B | A |
| 20 | B | A | B | A |
| 21 | B | A | B | A |
| 22 | B | A | B | B |
| 23 | C | A | C | A |
| 24 | B | A | B | A |
| 25 | a | A | A | A |
| 26 | B | A | A | A |
| 27 | B | A | A | A |
| 28 | B | B | A | A |
| 29 | B | A | A | A |
| 30 | A | A | C | A |
| 31 | ND | ND | ND | ND |
| 32 | B | A | B | B |
| 33 | B | A | B | A |
| 34 | D | C | D | C |
| 35 | D | C | C | A |
| 36 | A | A | A | A |
| 37 | B | A | B | A |
| 38 | A | A | A | A |
| 39 | B | A | C | A |
| 40 | C | C | C | B |
| 41 | A | A | B | A |
| 42 | B | A | B | A |
| 43 | A | A | C | A |
| 44 | C | A | C | A |
| 45 | C | A | C | A |
| 46 | B | B | C | C |
| 47 | B | A | A | A |
| 48 | B | A | B | A |
| 49 | B | A | B | A |
| 50 | B | A | C | A |
| 51 | B | A | C | B |
| 52 | A | A | B | A |
| 53 | A | A | A | A |
| 54 | C | C | D | C |
| 55 | B | A | C | A |
| 56 | C | B | B | A |
| 57 | B | B | C | B |
| 58 | A | A | C | B |
| 59 | C | A | B | A |
| 60 | C | B | B | A |
| 61 | C | C | C | C |
| 62 | C | A | C | A |
| 63 | A | A | A | A |
| 64 | A | A | B | A |
| 65 | C | A | D | B |
| 66 | C | A | D | B |
| 67 | C | B | C | A |
| 68 | B | A | B | A |
| 69 | C | A | C | B |
| 70 | A | A | A | A |
| 71 | B | A | B | A |
| 72 | A | A | B | A |
| 73 | B | A | B | A |
| 74 | A | A | C | B |
| 75 | D | B | D | A |
| 76 | B | A | B | A |
| 77 | C | A | D | B |
| 78 | C | B | D | C |
| 79 | B | A | B | A |
| 80 | A | A | C | B |
| 81 | B | A | B | A |
| 82 | C | B | C | A |
| 83 | A | A | A | A |
| 84 | B | A | C | A |
| 85 | C | B | C | A |
| 86 | D | B | D | C |
| 87 | B | C | B | A |
| 88 | C | B | D | C |
| 89 | B | A | B | A |
| 90 | C | B | B | A |
| 91 | B | A | C | A |
| 92 | C | C | D | C |
| 93 | B | A | B | A |
| 94 | C | B | B | A |
| 95 | A | A | A | A |
| 96 | B | B | C | C |
| 97 | B | A | C | B |
| 98 | ND | ND | ND | ND |
| 99 | A | A | B | A |
| 100 | A | A | C | A |
| 101 | D | C | D | C |
| 102 | B | B | B | B |
| 103 | A | A | D | C |
| 104 | B | B | B | A |
| 105 | C | C | C | B |
| 106 | C | B | C | B |
| 107 | B | B | B | B |
| 108 | B | B | B | C |
| 109 | C | C | C | B |
| 110 | B | A | C | A |
| 111 | A | A | B | A |
| 112 | A | A | B | A |
| 113 | B | A | C | A |
| 114 | D | C | D | B |
| 115 | C | B | C | A |
| 116 | D | C | D | C |
| 117 | D | D | D | C |
| 118 | D | C | C | C |
| 119 | C | B | B | A |
| 120 | C | A | B | A |
| 121 | A | A | B | A |
| 122 | D | B | C | A |
| 123 | B | A | C | B |
| 124 | A | A | C | A |
| 125 | C | B | C | A |
| 126 | B | B | B | A |
| 127 | B | A | B | A |
| 128 | C | B | D | B |
| 129 | B | A | C | A |
| 130 | C | B | C | B |
| 131 | C | B | D | B |
| 132 | B | A | B | A |
| 133 | B | A | B | A |
| 134 | B | A | C | A |
| 135 | D | C | D | C |
| 136 | A | B | B | B |
| 137 | C | B | A | A |
| 138 | A | A | A | A |
| 139 | C | A | D | A |
| 140 | B | A | C | A |
| 141 | A | A | B | A |
| 142 | C | B | C | B |
| 143 | B | A | A | A |
| 144 | A | A | B | A |
| 145 | D | C | D | D |
| 146 | A | A | B | A |
| 147 | C | C | C | B |
| 148 | B | A | C | B |
| 149 | C | B | C | C |
| 150 | C | C | C | B |
| 151 | C | B | C | B |
| 152 | B | B | C | C |
| 153 | C | A | B | A |
| 154 | C | A | C | A |
| 155 | C | C | B | A |
| 156 | ND | ND | ND | ND |
| 157 | B | A | B | A |

DC₅₀ values:
A: DC₅₀ < 50 nM;
B: 50 nM ≤ DC₅₀ < 150 nM;
C: 150 nM ≤ DC₅₀ < 500 nM;
D: 500 nM ≤ DC₅₀;
Eₘₐₓ values:
A: Eₘₐₓ < 10;
B: 10 ≤ Eₘₐₓ < 20;
C: 20 ≤ Eₘₐₓ < 50;
D: 50 ≤ Eₘₐₓ < 100

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I): 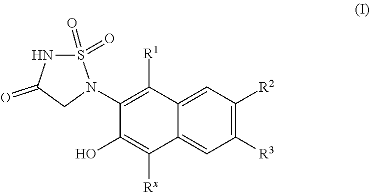
or a pharmaceutically acceptable salt thereof:
wherein:
R¹ is hydrogen or halogen;
R² is hydrogen, halogen, C1-C3 alkoxy, C3-C6 cycloalkoxy, C1-C3 haloalkoxy, C3-C5 halocycloalkoxy, C1-C3 alkyl, C1-C3 haloalkyl, C3-C6 cycloalkyl, or -L-Z;
R³ is hydrogen, halogen, C1-C3 alkoxy, C3-C5 cycloalkoxy, C1-C3 haloalkoxy, C3-C5 halocycloalkoxy, C1-C3 alkyl, C1-C3 haloalkyl, C3-C5 cycloalkyl, or -L-Z;
wherein one of R² and R³ is -L-Z and the other of R² and R³ is not -L-Z;
R^{x} is hydrogen or halogen;
L is —U—V—W—X—Y—;
U is a bond, —(NR⁴)—, —O—, C1-C3 alkylene, C2-C3 alkenylene, C2-C3 alkynylene, C3-C6 cycloalkylene, 4-10 membered heterocyclylene, 5-10 membered heteroarylene, —(C═O)NR⁴—, —NR⁴(C═O)—, —OR⁵—, —R⁵O—, —NR⁴R⁵—, —R⁵NR⁴—, or —(NR⁴)(C═O)(NR⁴)—;
each R⁴ is independently a hydrogen, C1-C6 alkyl, or C3-C5 cycloalkyl;
R⁵ is C1-C3 alkylene, C3-C7 cycloalkylene, or 4-12 membered heterocyclylene;
V is a bond, —(NR⁴)—, —O—, C1-C6 alkylene, C2-C6 alkenylene, —(C═O)NR⁴—, —(NR⁴)R⁵—, —(NR⁴)(C═O)—, —NH(C═O)NH—, —OR⁵—, —R⁵O—, 4-10 membered heterocyclylene, 5-10 membered heteroarylene, C6-C10 arylene, or C3-C6 cycloalkylene;
W is a bond, C1-C3 alkylene optionally substituted with hydroxyl, C3-C6 cycloalkylene, 4-12 membered heterocyclylene, —O—, —(NR⁴)—, —R⁵(NR⁴)—, —(NR⁴)R⁵—, —(NR⁴)(C═O)—, —R⁵(NR⁴)(C═O)—, —(C═O)(NR⁴)R⁵—, —R⁵(C═O)(NR⁴)—, —(C═O)(NR⁴)—, —R⁵(C═O)—, —(C═O)R⁵—, —(C═O)—, —(S═O)—, or —S(O₂)—;
X is a bond, C1-C3 alkylene, C3-C6 cycloalkylene, 4-12 membered heterocyclylene, C6-C10 arylene, 5-10 membered heteroarylene, —R⁵(NR⁴)(C═O)—, —(C═O)R⁵(NR⁴)—, —R⁵(C═O)(NR⁴)—, —(NR⁴)(C═O)R⁵—, —R⁵(C═O)(NR⁴)—, —(C═O)(NR⁴)R⁵—, —(NR⁴)R⁵(C═O)—, —R⁵(C═O)(NR⁴)R⁵—, —R⁵(NR⁴)(C═O)R⁵—, —(C═O)R⁵—, or —R⁵(C═O)—;
Y is R⁶, —R⁶(CR^{A}R^{B})ₚ-Q-, or -Q-(CR^{A}R^{B})ₚR⁶—;
Q is —(NR⁴)—, —O—, or —(CR^{A}R^{B})ₚ—;
p is 0, 1, 2, or 3;
R⁶ is C1-C3 alkylene, C3-C7 cycloalkylene, 4-12 membered heterocyclylene, C6-C10 arylene, or 5-10 membered heteroarylene;
wherein the heterocyclylene, heteroarylene, arylene, and cycloalkylene groups of U, V, W, X, and R⁶ are each optionally substituted with 1-3 substituents independently selected from fluoro, hydroxyl, C1-C6 alkoxy, and C1-C6 alkyl;
each R^{A} and R^{B} is independently hydrogen, fluoro, or C1-C6 alkyl; or
R^{A} and R^{B}, together with the carbon atom to which they are attached, come together to form a C3-C4 cycloalkyl; or
R^{A} and R^{B} combine to form oxo;
Z is selected from the group consisting of 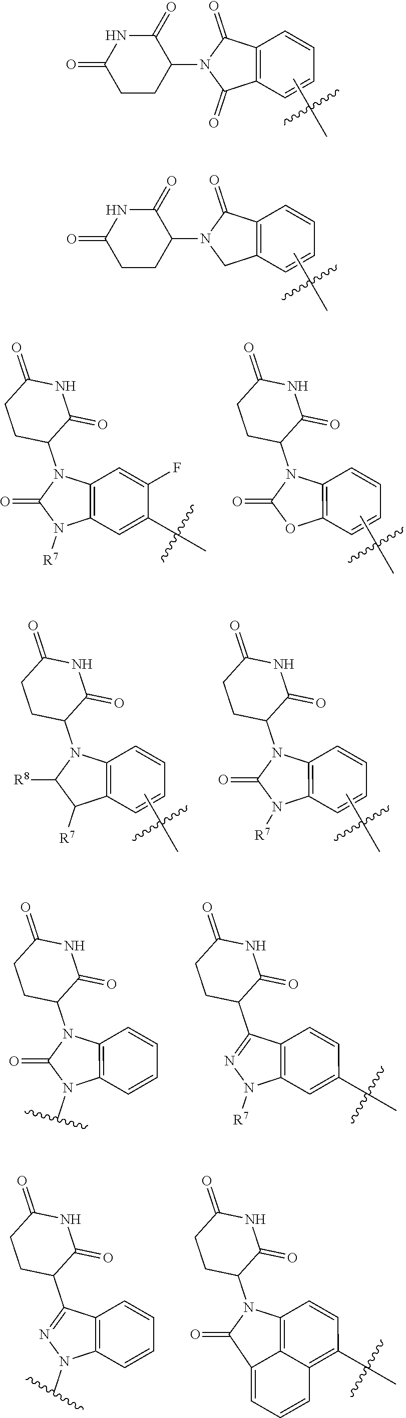 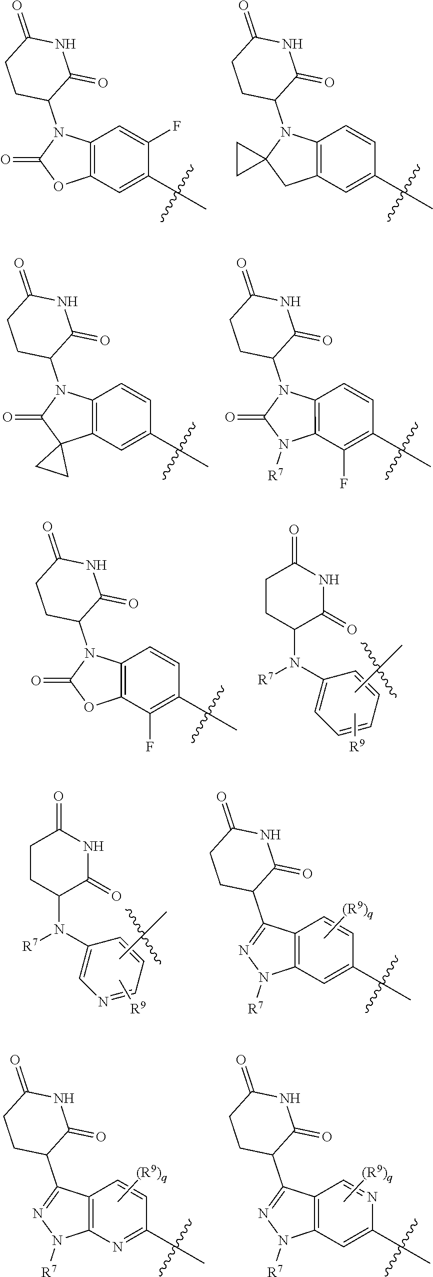 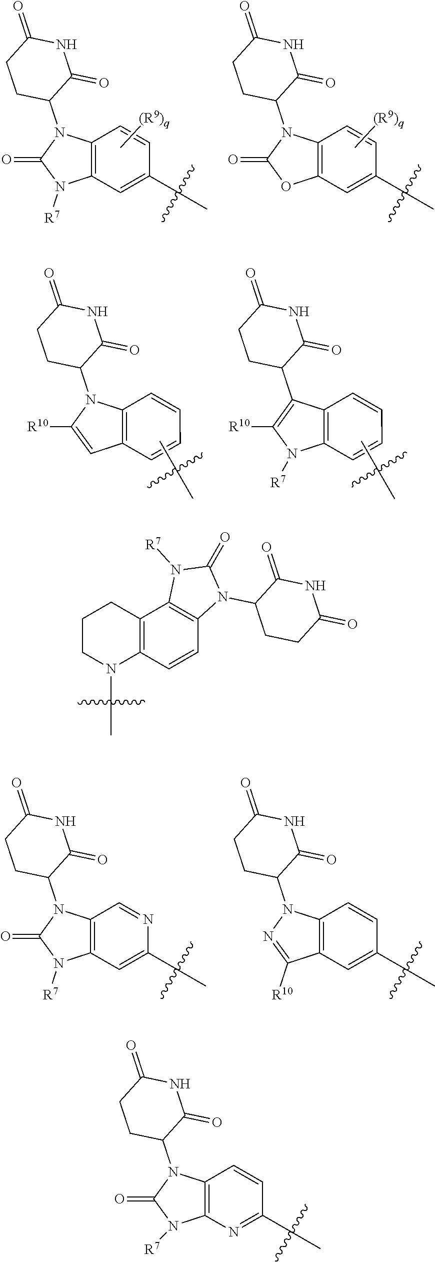 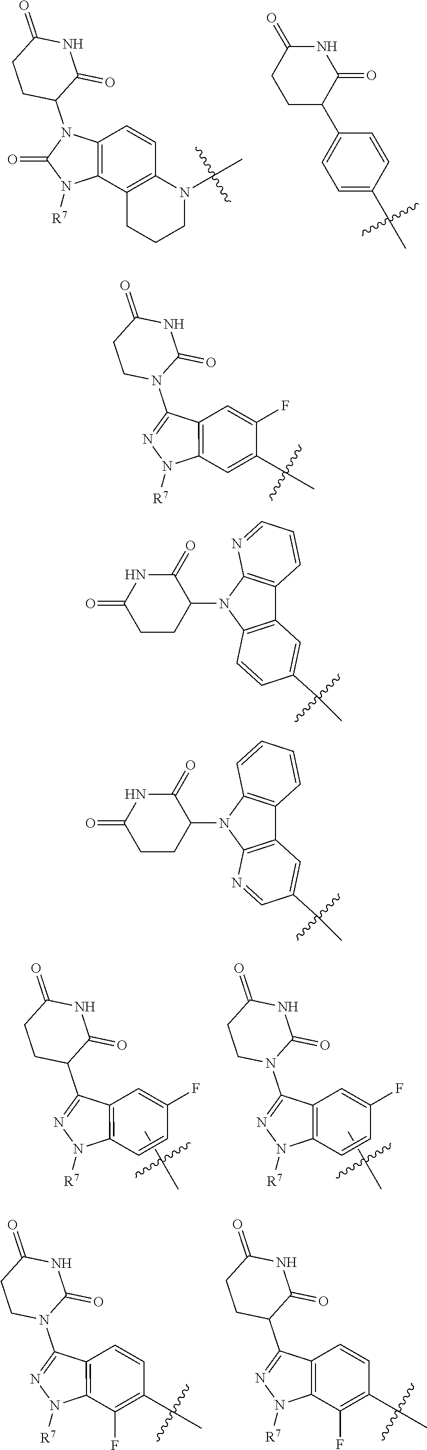
R⁷ is hydrogen, C1-C6 alkyl optionally substituted with one group selected from hydroxyl, cyano and C1-C6 alkoxy, C1-C6 haloalkyl, C3-C6 cycloalkyl, 4-6 membered heterocyclyl,
—(CR^{A}R^{B})(4-12 membered heterocyclyl), or —(CR^{A}R^{B})(C3-C6 cycloalkyl);
R⁸ is hydrogen or C1-C6 alkyl; and
each R⁹ is hydrogen, halogen, cyano, C1-C6 alkyl, C1-C6 haloalkyl, C1-C6 alkoxy, C1-C5 cycloalkoxy, 5-10 membered heteroaryloxy, or phenoxy;
q is 0, 1, or 2; and
each R¹⁰ is independently hydrogen, halogen, cyano, C1-C6 alkyl, C3-C6 cycloalkyl, or C1-C6 haloalkyl.

2. The compound of claim 1, wherein R¹ is halogen.

3. The compound of claim 1 or 2, wherein R¹ is —F.

4. The compound of claim 1 or 2, wherein R¹ is —Cl.

5. The compound of claim 1, wherein R¹ is hydrogen.

6. The compound of any one of claims 1-5, wherein R^{x} is halogen.

7. The compound of any one of claims 1-6, wherein R^{x} is —F.

8. The compound of any one of claims 1-6, wherein R^{x} is —Cl.

9. The compound of any one of claims 1-5, wherein R^{x} is hydrogen.

10. The compound of any one of claims 1-9, wherein R² is -L-Z.

11. The compound of any one of claims 1-10, wherein R³ is hydrogen.

12. The compound of any one of claims 1-10, wherein R³ is halogen.

13. The compound of any one of claims 1-10 or 12, wherein R³ is —F.

14. The compound of any one of claims 1-10 or 12, wherein R³ is —Cl.

15. The compound of any one of claims 1-10, wherein R³ is C1-C3 alkoxy.

16. The compound of any one of claims 1-10, wherein R³ is C1-C3 haloalkyl.

17. The compound of any one of claims 1-10, wherein R³ is C1-C3 haloalkoxy.

18. The compound of any one of claims 1-10, wherein R³ is C3-C5 cycloalkoxy.

19. The compound of any one of claims 1-10, wherein R³ is C3-C5 halocycloalkoxy.

20. The compound of any one of claims 1-10, wherein R³ is C1-C3 alkyl.

21. The compound of any one of claims 1-10, wherein R³ is C3-C5 cycloalkyl.

22. The compound of any one of claims 1-9, wherein R³ is -L-Z.

23. The compound of any one of claims 1-9 or 22, wherein R² is hydrogen.

24. The compound of any one of claims 1-9 or 22, wherein R² is halogen.

25. The compound of claim 24, wherein R² is —F.

26. The compound of claim 24, wherein R² is —Cl.

27. The compound of any one of claims 1-9 or 22, wherein R² is C1-C3 alkoxy.

28. The compound of any one of claims 1-9 or 22, wherein R² is C1-C3 haloalkyl.

29. The compound of any one of claims 1-9 or 22, wherein R² is C1-C3 haloalkoxy.

30. The compound of any one of claims 1-9 or 22, wherein R² is C3-C5 cycloalkoxy.

31. The compound of any one of claims 1-9 or 22, wherein R² is C3-C5 halocycloalkoxy.

32. The compound of any one of claims 1-9 or 22, wherein R² is C1-C3 alkyl.

33. The compound of any one of claims 1-9 or 22, wherein R² is C3-C5 cycloalkyl.

34. The compound of claim 33, wherein R¹ is —F; and R^{x} is hydrogen, —F, or —Cl.

35. The compound of claim 33 or 34, wherein R¹ is —F; and R^{x} is hydrogen or —F.

36. The compound of claim 34 or 35, wherein R^{x} is hydrogen.

37. The compound of any one of claims 1-9 or 22-36, wherein R³ is -L-Z.

38. The compound of claim 37, wherein R² is hydrogen.

39. The compound of any one of claims 1-9 or 11-21, wherein R² is -L-Z.

40. The compound of claim 39, wherein R³ is hydrogen.

41. The compound of claim 1, wherein R¹ is —F; R^{x} is hydrogen; R² is -L-Z; and R³ is hydrogen.

42. The compound of claim 1, wherein R¹ is —F; R^{x} is hydrogen; R² is hydrogen; and R³ is -L-Z.

43. The compound of any one of claims 1-42, wherein U is —(NR⁴)—, —NHR⁵—, or —R⁵NH—.

44. The compound of any one of claims 1-43, wherein U is —(NR⁴)—.

45. The compound of claim 44, wherein R⁴ is hydrogen.

46. The compound of claim 44, wherein R⁴ is C1-C6 alkyl.

47. The compound of any one of claims 1-42, wherein U is —O—, —OR⁵—, or —R⁵O—.

48. The compound of claim 47, wherein U is —O—.

49. The compound of any one of claims 1-42, wherein U is —NR⁴(C═O)—, —(C═O)NR⁴—, or —(NR⁴)(C═O)(NR⁴)—.

50. The compound of any one of claims 1-42 or 49, wherein U is —NR⁴(C═O)—.

51. The compound of any one of claims 1-42 or 49, wherein U is —(NR⁴)(C═O)(NR⁴)—.

52. The compound of any one of claims 49-51, wherein each R⁴ within U is independently hydrogen or C1-C6 alkyl.

53. The compound of any one of claims 1-42, wherein U is C1-C3 alkylene, C2-C3 alkenylene, or C2-C3 alkynylene.

54. The compound of claim 53, wherein U is C1-C3 alkylene.

55. The compound of claim 53, wherein U is C2-C3 alkenylene.

56. The compound of claim 53, wherein U is C2-C3 alkynylene.

57. The compound of any one of claims 1-42, wherein U is C3-C6 cycloalkylene, 4-10 membered heterocyclylene, or 5-10 membered heteroarylene; each optionally substituted with 1-3 substituents independently selected from fluoro, hydroxyl, C1-C6 alkoxy, and C1-C6 alkyl.

58. The compound of any one of claims 1-42, wherein U is a bond.

59. The compound of any one of claims 1-58, wherein V is C1-C6 alkylene or C2-C6 alkenylene.

60. The compound of any one of claims 1-59, wherein V is C1-C6 alkylene.

61. The compound of any one of claims 1-60, wherein V is C1-C3 alkylene.

62. The compound of any one of claims 1-61, wherein V is methylene or ethylene.

63. The compound of any one of claims 1-58, wherein V is 4-10-membered heterocyclylene, 5-10 membered heteroarylene, C6-C10 arylene, or C3-C6 cycloalkylene; each optionally substituted with 1-3 substituents independently selected from fluoro, hydroxyl, C1-C6 alkoxy, and C1-C6 alkyl.

64. The compound of any one of claims 1-58 or 63, wherein V is 4-10-membered heterocyclylene optionally substituted with 1-3 substituents independently selected from fluoro, hydroxyl, C1-C6 alkoxy, and C1-C6 alkyl.

65. The compound of any one of claims 1-58 or 63-64, wherein V is 4-6-membered heterocyclylene optionally substituted with 1-3 substituents independently selected from fluoro, hydroxyl, C1-C6 alkoxy, and C1-C6 alkyl.

66. The compound of any one of claims 1-58 or 63-64, wherein V is 4-10-membered heterocyclylene substituted with 1-3 substituents independently selected from fluoro, hydroxyl, C1-C6 alkoxy, and C1-C6 alkyl.

67. The compound of any one of claims 1-58, 63-64 or 66, wherein V is 4-6-membered heterocyclylene substituted with 1-3 substituents independently selected from fluoro, hydroxyl, C1-C6 alkoxy, and C1-C6 alkyl.

68. The compound of any one of claims 1-58, 63-64 or 66-67, wherein V is 4-6-membered heterocyclylene substituted with methyl, hydroxyl, methoxy, or 1 or 2 fluoros.

69. The compound of any one of claims 1-58 or 63-64, wherein V is 4-10-membered heterocyclylene.

70. The compound of any one of claims 1-58, 63-64 or 69, wherein V is 4-6-membered heterocyclylene.

71. The compound of any one of claims 1-58 or 70, wherein V is selected from the group consisting of:

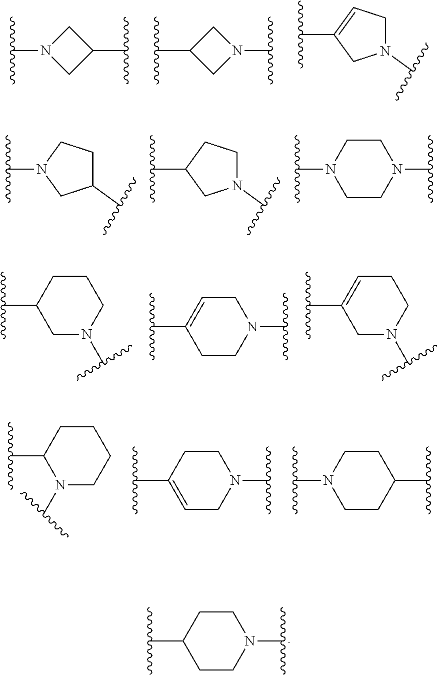

72. The compound of any one of claims 1-58 or 63, wherein V is 5-10-membered heteroarylene.

73. The compound of any one of claims 1-58, 63, or 72, wherein V is 5-6-membered heteroarylene.

74. The compound of any one of claims 1-58, 63, or 72-73, wherein V is 5-membered heteroarylene.

75. The compound of claim 74, wherein V is selected from the group consisting of:

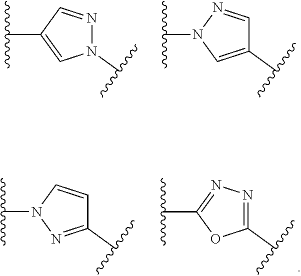 [SMILES: *c1cnn(*)c1 ; *c1cnn(*)c1 ; *c1ccn(*)n1 ; *c1nnc(*)o1]

76. The compound of any one of claims 1-58 or 63, wherein V is C3-C6 cycloalkylene.

77. The compound of claim 76, wherein V is selected from the group consisting of cyclobutylene, cyclopentylene, and cyclohexylene.

78. The compound of any one of claims 1-58, wherein V is —(C═O)NR⁴—, —(NR⁴)R⁵—, —(NR⁴)(C═O)—, or —NH(C═O)NH—.

79. The compound of any one of claims 1-58, wherein V is —(NR⁴)— or —(NR⁴)R⁵—.

80. The compound of any one of claims 1-58, wherein V is —O—, —OR⁵—, or —R⁵O—.

81. The compound of any one of claims 1-58, wherein V is a bond.

82. The compound of any one of claims 1-81, wherein W is a bond.

83. The compound of any one of claims 1-81, wherein W is C1-C3 alkylene optionally substituted with hydroxyl.

84. The compound of any one of claims 1-81 or 83, wherein W is C1-C3 alkylene.

85. The compound of any one of claims 1-81 or 83, wherein W is C1-C3 alkylene substituted with hydroxyl.

86. The compound of any one of claims 1-81, wherein W is C3-C6 cycloalkylene or 4-12 membered heterocyclylene; each optionally substituted with 1-3 substituents independently selected from fluoro, hydroxyl, C1-C6 alkoxy, and C1-C6 alkyl.

87. The compound of any one of claims 1-81, wherein W is —O—, —(NR⁴)—, —R⁵(NR⁴)—, or —(NR⁴)R⁵—.

88. The compound of any one of claims 1-81 or 87, wherein W is —O— or —(NR⁴)—.

89. The compound of claim 88, wherein each R⁴ within W is hydrogen.

90. The compound of any one of claims 1-81, wherein W is —(NR⁴)(C═O)—, —R⁵(NR⁴)(C═O)—, —(C═O)R⁵(NR⁴)—, —R⁵(C═O)(NR⁴)—, or —(C═O)(NR⁴)—.

91. The compound of any one of claims 1-81 or 90, wherein W is —(NR⁴)(C═O)—.

92. The compound of any one of claims 1-81 or 90, wherein W is —R⁵(NR⁴)(C═O)—.

93. The compound of any one of claims 1-81 or 90, wherein W is —(C═O)(NR⁴)—.

94. The compound of any one of claims 90-93, wherein R⁴ within W is hydrogen.

95. The compound of any one of claims 90-93, wherein R⁴ within W is C1-C3 alkyl.

96. The compound of any one of claim 90 or 92, wherein each R⁵ within W is independently C1-C3 alkylene.

97. The compound of any one of claims 1-81, wherein W is —R⁵(C═O)—, —(C═O)R⁵—, —(C═O)—, —(S═O)—, or —S(O₂)—.

98. The compound of any one of claims 1-81 or 967, wherein W is —(C═O)—.

99. The compound of any one of claims 1-81 or 97, wherein W is —R⁵(C═O)— or —(C═O)R⁵—, and wherein R⁵ is C1-C3 alkylene or C3-C7 cycloalkylene.

100. The compound of any one of claims 1-81 or 97, wherein W is —(S═O)—.

101. The compound of any one of claims 1-81 or 97, wherein W is —S(O₂)—.

102. The compound of any one of claims 1-101, wherein X is C3-C6 cycloalkylene, 4-12 membered heterocyclylene, C6-C10 arylene, or 5-10 membered heteroarylene; each optionally substituted with 1-3 substituents independently selected from fluoro, hydroxyl, C1-C6 alkoxy, and C1-C6 alkyl.

103. The compound of any one of claims 1-102, wherein X is C3-C6 cycloalkylene or 4-12 membered heterocyclylene; each optionally substituted with 1-3 substituents independently selected from fluoro, hydroxyl, C1-C6 alkoxy, and C1-C6 alkyl.

104. The compound of any one of claims 1-103, wherein X is 4-10 membered heterocyclylene optionally substituted with 1-3 substituents independently selected from fluoro, hydroxyl, C1-C6 alkoxy, and C1-C6 alkyl.

105. The compound of any one of claims 1-104, wherein X is 4-6 membered heterocyclylene optionally substituted with 1-3 substituents independently selected from fluoro, hydroxyl, C1-C6 alkoxy, and C1-C6 alkyl.

106. The compound of any one of claims 1-105, wherein X is selected from the group consisting of:

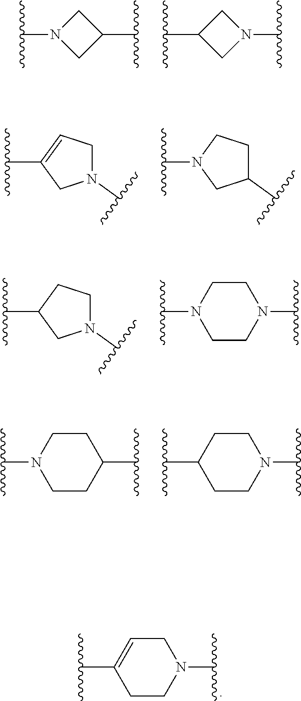

107. The compound of claim 106, wherein X is

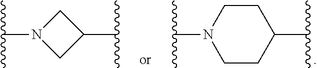 [SMILES: *C1CN(*)C1 ; *C1CCN(*)CC1]

108. The compound of any one of claims 1-101 or 104, wherein X is 5-10 membered heteroarylene optionally substituted with 1-3 substituents independently selected from fluoro, hydroxyl, C1-C6 alkoxy, and C1-C6 alkyl.

109. The compound of any one of claims 1-102, wherein X is 5 membered heteroarylene optionally substituted with 1-3 substituents independently selected from fluoro, hydroxyl, C1-C6 alkoxy, and C1-C6 alkyl.

110. The compound of any one of claims 1-102 or 108-109, wherein X is selected from the group consisting of:

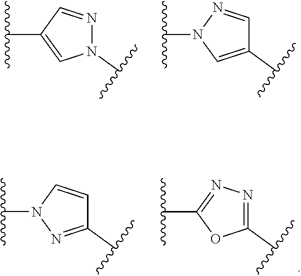 [SMILES: *c1cnn(*)c1 ; *c1cnn(*)c1 ; *c1ccn(*)n1 ; *c1nnc(*)o1]

111. The compound of any one of claims 1-101, wherein X is selected from the group consisting of —R⁵(NR⁴)(C═O)—, —(C═O)R⁵(NR⁴)—, —R⁵(C═O)(NR⁴)—, —(NR⁴)(C═O)R⁵—, —R⁵(C═O)(NR⁴)—, —(C═O)(NR⁴)R⁵—, —(NR⁴)R⁵(C═O)—, —R⁵(C═O)(NR⁴)R⁵—, or —R⁵(NR⁴)(C═O)R⁵—.

112. The compound of any one of claims 1-101 or 111, wherein X is —(C═O)R⁵— or —R⁵(C═O)—.

113. The compound of claim 112, wherein each R⁴ within X is independently hydrogen or C1-C3 alkyl.

114. The compound of any one of claims 111-113, wherein each R⁴ within X is hydrogen.

115. The compound of any one of claims 111-113, wherein each R⁵ within X is independently C1-C3 alkylene.

116. The compound of any one of claims 1-101, wherein X is C1-C3 alkylene.

117. The compound of any one of claims 1-101 or 116, wherein X is methylene or ethylene.

118. The compound of any one of claims 1-101, wherein X is a bond.

119. The compound of any one of claims 1-42, wherein U is —NR⁴(C═O)— or —(C═O)NR⁴—; V is a bond, C1-C6 alkylene, or C3-C6 cycloalkylene; W is a bond; and X is a bond.

120. The compound of any one of claims 1-42, wherein U is —(NR⁴)(C═O)(NR⁴)—, —NR⁴(C═O)—, or —(C═O)NR⁴—; V is a bond, C1-C6 alkylene, or C3-C6 cycloalkylene; W is a bond; and X is a bond, C6-C10 arylene, or C1-C3 alkylene.

121. The compound of claim 119 or 120, wherein U is —NR⁴(C═O)—.

122. The compound of claim 119 or 120, wherein U is —(C═O)NR⁴—.

123. The compound of claim 119 or 120, wherein U is —(NR⁴)(C═O)(NR⁴)—.

124. The compound of any one of claims 119-123, wherein V is a bond.

125. The compound of any one of claims 119-123, wherein V is C1-C3 alkylene.

126. The compound of claim 125, wherein V is methylene or ethylene.

127. The compound of any one of claims 1-42, wherein U is —O—; V is C1-C6 alkylene, C3-C6 cycloalkylene, or 4-10-membered heterocyclylene; W is a bond, —R⁵(C═O)—, —(C═O)R⁵—, —C(═O)—, —N(R⁴)—, —C(═O)NR⁴—, —NR⁴C(═O)—, or —NR⁴C(═O)R⁵—.

128. The compound of claim 127, wherein V is C1-C6 alkylene.

129. The compound of claim 127 or 128, wherein V is C1-C3 alkylene.

130. The compound of claim 129, wherein V is methylene or ethylene.

131. The compound of claim 127, wherein V is 4-10 membered heterocyclylene.

132. The compound of any one of claims 127-131, wherein W is —C(═O)—, —C(═O)R⁵—, —C(═O)NR⁴— or a bond.

133. The compound of any one of claims 127-132, wherein W is —NR⁴C(═O)— or —(C═O)R⁵—.

134. The compound of any one of claims 127-133, wherein R⁵ is C1-C3 alkylene or C3-C7 cycloalkylene.

135. The compound of any one of claims 127-134, wherein each R⁵ within W is —CH₂—.

136. The compound of any one of claims 127-134, wherein each R⁵ within W is independently C3-C7 cycloalkylene.

137. The compound of any one of claims 1-42, wherein U is a bond or 4-10 membered heterocyclylene; V is 4-10 membered heterocyclylene, C1-C6 alkylene or a bond; W is —C(═O)— or —C(═O)R⁵—; and X is a bond or C1-C3 alkylene.

138. The compound of claim 137, wherein U is a bond.

139. The compound of claim 137, wherein U is 4-10 membered heterocyclylene.

140. The compound of any one of claims 137-139, wherein V is a bond.

141. The compound of any one of claims 137-139, wherein V is 4-10 membered heterocyclylene.

142. The compound of any one of claims 137-141, wherein W is —C(═O)—.

143. The compound of any one of claims 137-141, wherein W is —C(═O)R⁵—.

144. The compound of claim 143, wherein each R⁵ within W is independently C1-C3 alkylene or C3-C7 cycloalkylene.

145. The compound of any one of claims 1-42, wherein U is a bond, C1-C3 alkylene, C2-C3 alkenylene, or C2-C3 alkynylene; V is a bond or 4-10 membered heterocyclylene; W is a bond, C(═O), or —C(═O)R⁵—; and X is a bond, C1-C3 alkylene or C6-C10 arylene.

146. The compound of claim 145, wherein U is a bond.

147. The compound of claim 145, wherein U is C2-C3 alkenylene.

148. The compound of any one of claims 145-147, wherein W is a bond.

149. The compound of any one of claims 145-147, wherein W is C(═O).

150. The compound of any one of claims 145-147, wherein W is C(═O)R⁵.

151. The compound of any one of claims 145-150, wherein X is a bond.

152. The compound of any one of claims 145-150, wherein X is C6-C10 arylene.

153. The compound of any one of claims 145-150, wherein X is C1-C3 alkylene.

154. The compound of any one of claims 1-42, wherein U is —NR⁴(C═O)—, —(C═O)NR⁴—, or —(NR⁴)(C═O)(NR⁴)—; V is C1-C6 alkylene; W is C1-C3 alkylene or a bond; and X is a bond.

155. The compound of claim 154, wherein U is —NR⁴(C═O)—.

156. The compound of claim 154, wherein U is —(C═O)NR⁴—.

157. The compound of claim 154, wherein U is —(NR⁴)(C═O)(NR⁴)—.

158. The compound of any one of claims 154-157, wherein each R⁴ within U is hydrogen.

159. The compound of any one of claims 154-158, wherein W is methylene.

160. The compound of any one of claims 154-158, wherein W is ethylene.

161. The compound of any one of claims 154-158, wherein W is n-propylene.

162. The compound of any one of claims 154-158, wherein W is iso-propylene.

163. The compound of any one of claims 119-162, wherein Y is R⁶.

164. The compound of claim 163, wherein R⁶ is 4-12 membered heterocyclylene optionally substituted with 1-3 substituents independently selected from fluoro, hydroxyl, C1-C6 alkoxy, and C1-C6 alkyl.

165. The compound of any one of claims 163-164, wherein R⁶ is 4-8 membered heterocyclylene optionally substituted with 1-3 substituents independently selected from fluoro, hydroxyl, C1-C6 alkoxy, and C1-C6 alkyl.

166. The compound of any one of claims 163-164, wherein R⁶ is 4-12 membered heterocyclylene.

167. The compound of any one of claims 163-166, wherein R⁶ is 4-8 membered heterocyclylene.

168. The compound of any one of claims 163-164, wherein R⁶ is selected from the group consisting of:

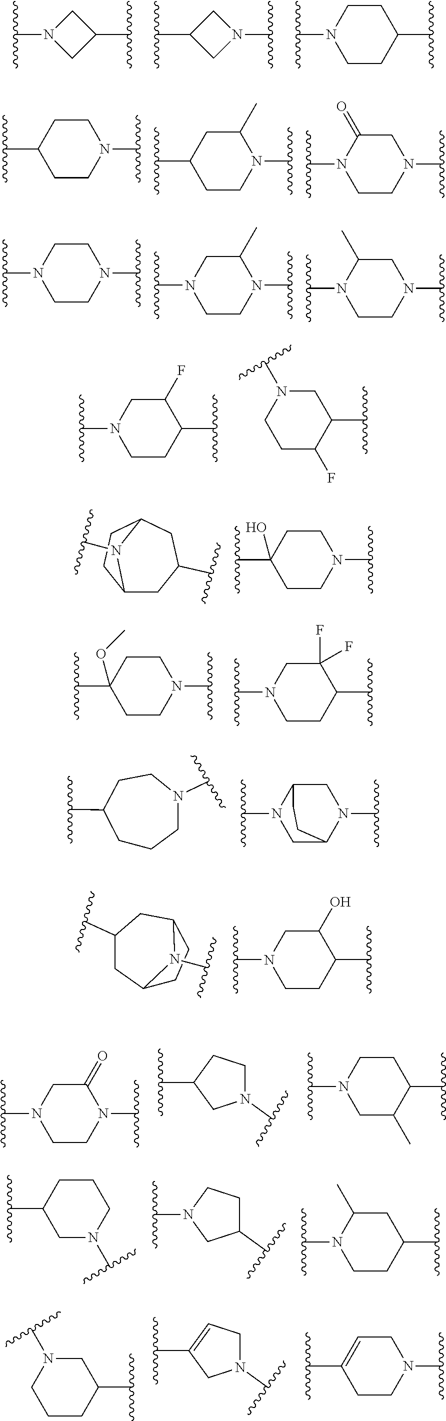

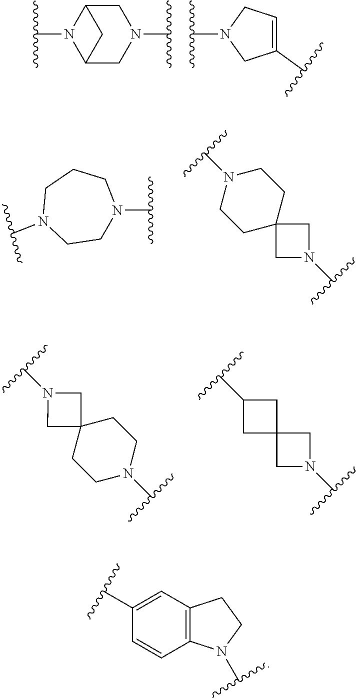

169. The compound of any one of claims 163-164, wherein R⁶ is

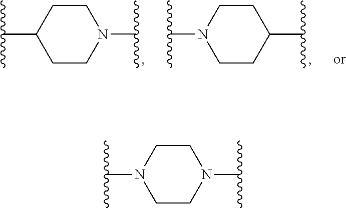

170. The compound of any one of claims 163-169, wherein R⁶ is

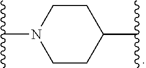 [SMILES: *C1CCN(*)CC1]

171. The compound of any one of claims 163-164, wherein R⁶ is 4-12 membered heterocyclylene substituted with 1-3 substituents independently selected from fluoro, hydroxyl, C1-C6 alkoxy, and C1-C6 alkyl.

172. The compound of any one of claims 163-164 or 171, wherein R⁶ is 4-8 membered heterocyclylene substituted with 1-3 substituents independently selected from fluoro, hydroxyl, C1-C6 alkoxy, and C1-C6 alkyl.

173. The compound of any one of claims 163-164 or 171-172, wherein R⁶ is 4-12 membered heterocyclylene substituted with methyl, hydroxyl, methoxy, oxo, or 1 or 2 fluoros.

174. The compound of any one of claims 163-164 or 171-173, wherein R⁶ is 4-8 membered heterocyclylene substituted with methyl, hydroxyl, methoxy, oxo, or 1 or 2 fluoros.

175. The compound of any one of claims 163-164 or 171-174, wherein R⁶ is selected from the group consisting of:

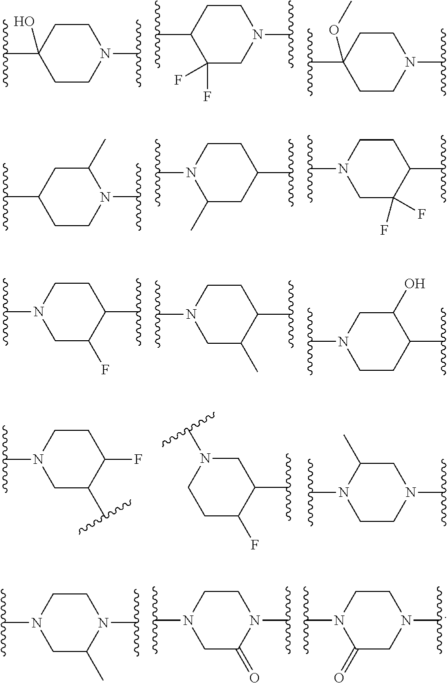

176. The compound of any one of claims 163-164, wherein R⁶ is 7-12 membered bicyclic heterocyclylene optionally substituted with 1-3 substituents independently selected from fluoro, hydroxyl, C1-C6 alkoxy, and C1-C6 alkyl.

177. The compound of claim 176, wherein R⁶ is 7-12 membered bicyclic spirocyclic heterocyclylene optionally substituted with 1-3 substituents independently selected from fluoro, hydroxyl, C1-C6 alkoxy, and C1-C6 alkyl.

178. The compound of any one of claims 176-177, wherein R⁶ is 7-12 membered bicyclic spirocyclic heterocyclylene.

179. The compound of any one of claims 176-178, wherein R⁶ is

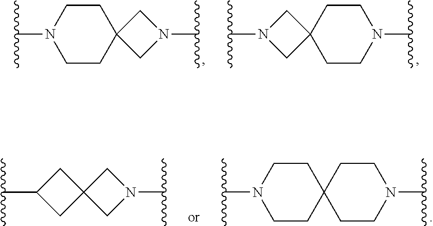

180. The compound of claim 163, wherein R⁶ is 5-10 membered heteroarylene optionally substituted with 1-3 substituents independently selected from fluoro, hydroxyl, C1-C6 alkoxy, and C1-C6 alkyl.

181. The compound of claim 163 or 180, wherein R⁶ is 5-6 membered heteroarylene optionally substituted with 1-3 substituents independently selected from fluoro, hydroxyl, C1-C6 alkoxy, and C1-C6 alkyl.

182. The compound of any one of claim 163 or 180, wherein R⁶ is 5-6 membered heteroarylene.

183. The compound of claim 163 or 180, wherein R⁶ is selected from the group consisting of:

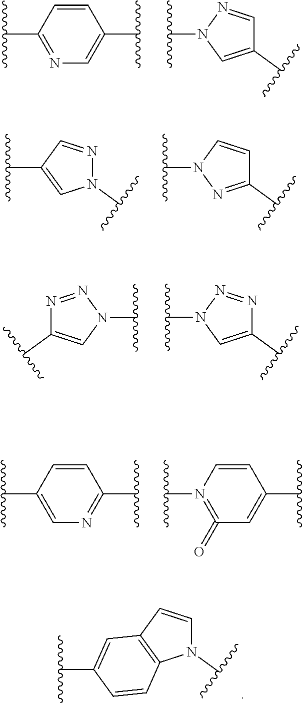

184. The compound of claim 163 or 180, wherein R⁶ is 5-10 membered heteroarylene substituted with 1-3 substituents independently selected from fluoro, hydroxyl, C1-C6 alkoxy, and C1-C6 alkyl.

185. The compound of claim 163 or 184, wherein R⁶ is C1-C3 alkylene.

186. The compound of claim 163, wherein —Y— is —R⁶(CR^{A}R^{B})ₚ-Q-.

187. The compound of claim 186, wherein -Q- is —(NR⁴)—.

188. The compound of claim 187, wherein R⁴ is hydrogen.

189. The compound of claim 187, wherein R⁴ is C1-C3 alkyl.

190. The compound of claim 186, wherein -Q- is —O—.

191. The compound of any one of claims 186-190, wherein p is 1.

192. The compound of any one of claims 186-190, wherein p is 2.

193. The compound of any one of claims 186-193, wherein each R^{A} and R^{B} are independently hydrogen, fluoro, or C1-C3 alkyl.

194. The compound of any one of claims 186-193, wherein one pair of R^{A} and R^{B}, on the same carbon, combine to form oxo.

195. The compound of any one of claims 186-193, wherein each R^{A} and R^{B} are hydrogen.

196. The compound of any one of claims 186-194, wherein 1 or 2 of R^{A} and R^{B} are independently fluoro or C1-C3 alkyl; and each remaining R^{A} and R^{B} is hydrogen.

197. The compound of any one of claims 186-194, wherein one pair of R^{A} and R^{B}, on the same carbon, combine to form oxo; and each remaining R^{A} and R^{B}, if present, are hydrogen.

198. The compound of any one of claims 186-190, wherein p is 0.

199. The compound of claim 186-198, wherein Y is —R⁶(CR^{A}R^{B})ₚ-Q-; and p is 0.

200. The compound of claim 163, wherein Y is —R⁶NR⁴— or —R⁶O—.

201. The compound of claim 163 or 200, wherein Y is —R⁶NR⁴—.

202. The compound of claim 163 or 200, wherein Y is —R⁶O—.

203. The compound of any one of claims 186-192, wherein Y is R⁶(CR^{A}R^{B})ₚ-Q-; p is 1 or 2; and each R^{A} and R^{B} are hydrogen.

204. The compound of claim 203, wherein Y is —R⁶CH₂—O— or —R⁶CH₂—N(R⁴)—.

205. The compound of claim 203 or 204, wherein Y is —R⁶CH₂—O—.

206. The compound of claim 203 or 204, wherein Y is —R⁶CH₂—NH.

207. The compound of any one of claims 186-192, wherein Y is —R⁶(CR^{A}R^{B})ₚ-Q-, p is 1 or 2, and each R^{A} and R^{B} are independently hydrogen or C1-C3 alkyl; or one pair of R^{A} and R^{B}, together with the carbon atom to which they are attached, come together to form a C3-C4 cycloalkyl, and each remaining R^{A} and R^{B}, if present, are hydrogen.

208. The compound of claim 207, wherein the —(CR^{A}R^{B})ₚ-Q- portion of Y is selected from the group consisting of:

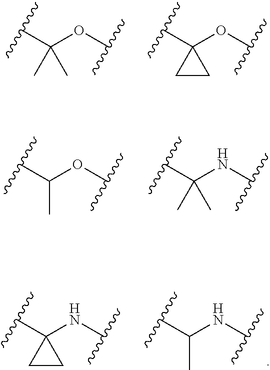

209. The compound of any one of claims 186-192, wherein Y is —R⁶C(═O)(CR^{A}R^{B})-Q-; and each R^{A} and R^{B} are independently hydrogen, fluoro, or C1-C3 alkyl.

210. The compound of claim 209, wherein the —(CR^{A}R^{B})ₚ-Q- portion of Y is selected from the group consisting of:

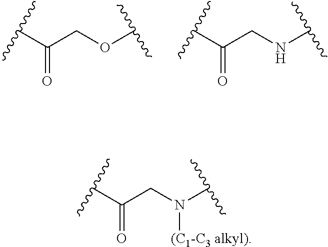

211. The compound of claim 163, wherein R⁶ is 4-12 membered heterocyclylene optionally substituted with 1-3 substituents independently selected from fluoro, hydroxyl, C1-C6 alkoxy, and C1-C6 alkyl.

212. The compound of any one of claims 163 and 211, wherein R⁶ is 4-8 membered heterocyclylene optionally substituted with 1-3 substituents independently selected from fluoro, hydroxyl, C1-C6 alkoxy, and C1-C6 alkyl.

213. The compound of claim 212, wherein R⁶ is selected from the group consisting of:

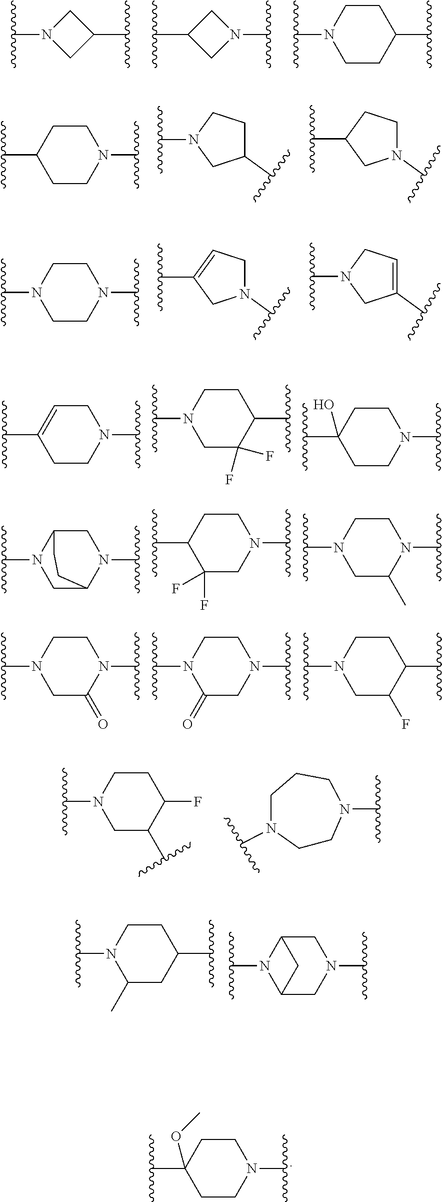

214. The compound of any one of claims 211-213, wherein R⁶ is

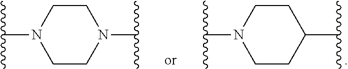 [SMILES: *N1CCN(*)CC1 ; *C1CCN(*)CC1]

215. The compound of any one of claims 211-213, wherein R⁶ is

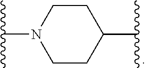 [SMILES: *C1CCN(*)CC1]

216. The compound of claim 211, wherein R⁶ is 7-12 membered bicyclic heterocyclylene.

217. The compound of claim 216, wherein R⁶ is 7-12 membered bicyclic spirocyclic heterocyclylene.

218. The compound of claim 216, wherein R⁶ is 9-12 membered bicyclic spirocyclic heterocyclylene.

219. The compound of claim 216 or 218, wherein R⁶ is 2

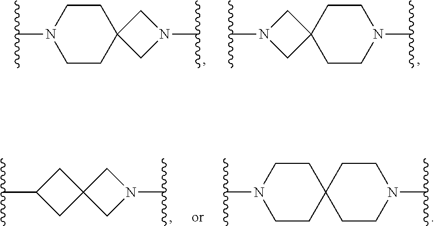

220. The compound of claim 163, wherein R⁶ is 5-10 membered heteroarylene optionally substituted with 1-3 substituents independently selected from fluoro, hydroxyl, C1-C6 alkoxy, and C1-C6 alkyl.

221. The compound of claim 163 or 220, wherein R⁶ is 5-6 membered heteroarylene optionally substituted with 1-3 substituents independently selected from fluoro, hydroxyl, C1-C6 alkoxy, and C1-C6 alkyl.

222. The compound of any one of claims 163 or 220-221, wherein R⁶ is 5-6 membered heteroarylene.

223. The compound of any one of claims 163 or 220-222 wherein R⁶ is 5-membered heteroarylene.

224. The compound of claim 223, wherein R⁶ is triazolylene or pyrazolylene.

225. The compound of any one of claim 163 or 222, wherein R⁶ is 6-membered heteroarylene.

226. The compound of claim 225, wherein R⁶ is pyridinylene.

227. The compound of any one of claims 163 or 222-226, wherein R⁶ is selected from the group consisting of:

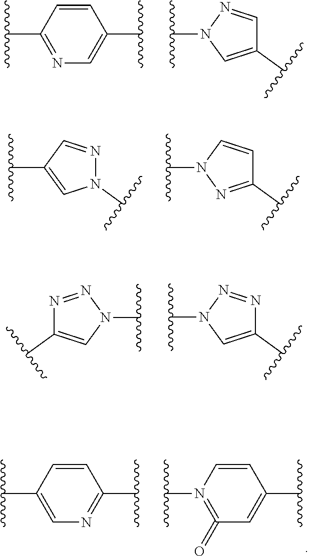

228. The compound of claim 227, wherein R⁶ is selected from the group consisting of:

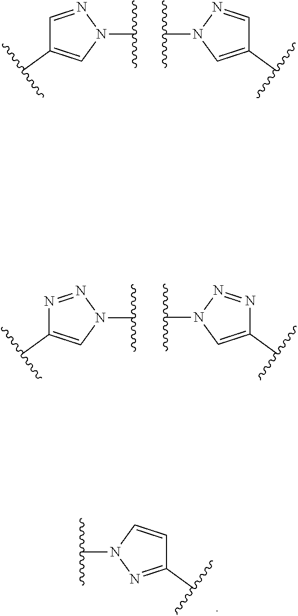

229. The compound of claim 227, wherein R⁶ is selected from the group consisting of:

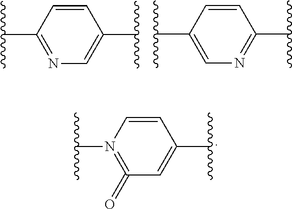

230. The compound of claim 163, wherein R⁶ is C6-C10 arylene.

231. The compound of any one of claims 163 or 230, wherein R⁶ is phenylene.

232. The compound of any one of claims 163 or 230, wherein R⁶ is naphthylene.

233. The compound of any one of claims 1-232, wherein Z is selected from the group consisting of:

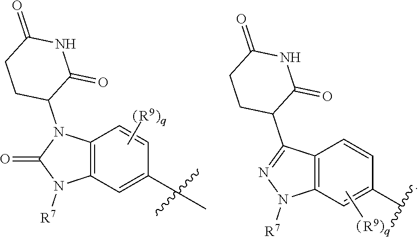

234. The compound of any one of claims 1-232, wherein Z is:

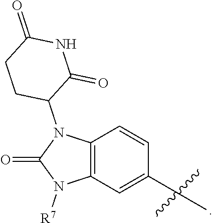

235. The compound of any one of claims 1-232, wherein Z is:

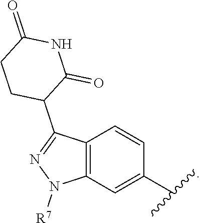

236. The compound of any one of claims 1-232, wherein Z is

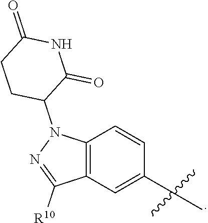

237. The compound of any one of claims 1-232, wherein Z is:

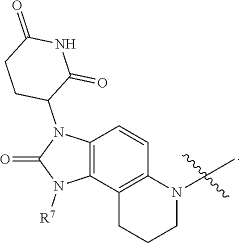

238. The compound of any one of claims 1-232, wherein Z is selected from the PGP-601C3 group consisting of:

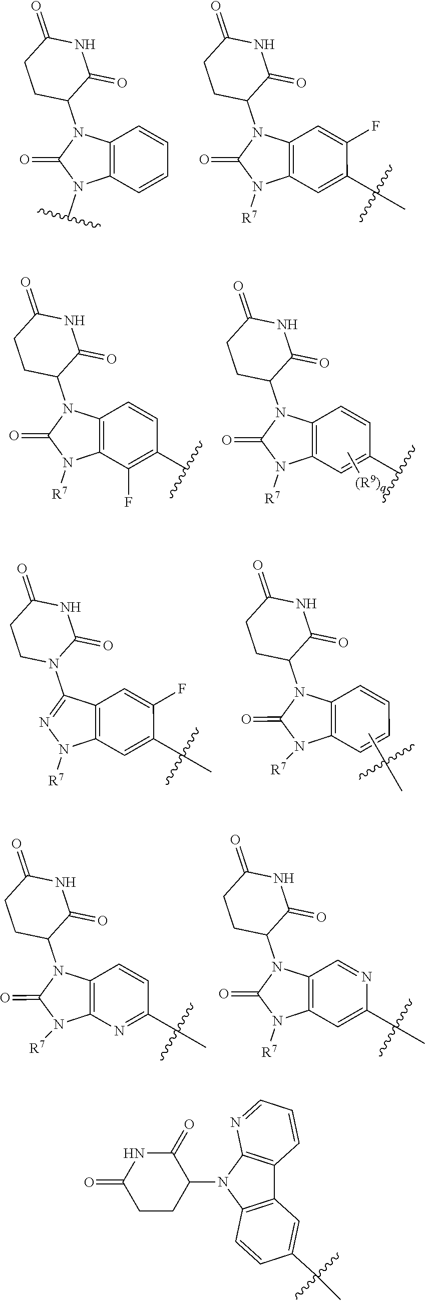

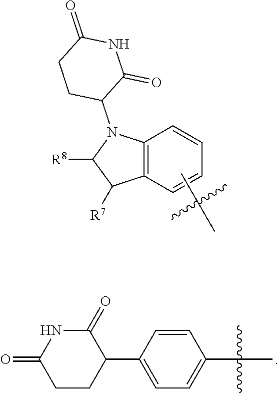

239. The compound of any one of claims 1-232, wherein Z is:

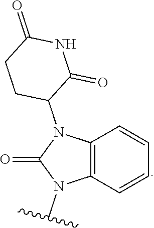 [SMILES: *n1c(=O)n(C2CCC(=O)NC2=O)c2ccccc21]

240. The compound of any one of claims 1-232, wherein Z is:

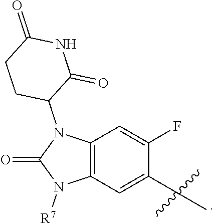

241. The compound of any one of claims 1-232, wherein Z is:

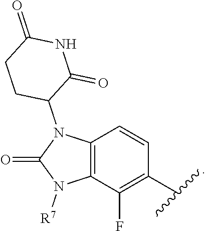

242. The compound of any one of claims 1-232, wherein Z is:

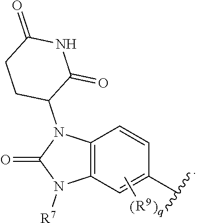

243. The compound of any one of claims 1-232, wherein Z is:

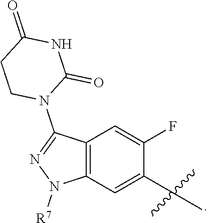

244. The compound of any one of claims 1-232, wherein Z is selected from the group consisting of:

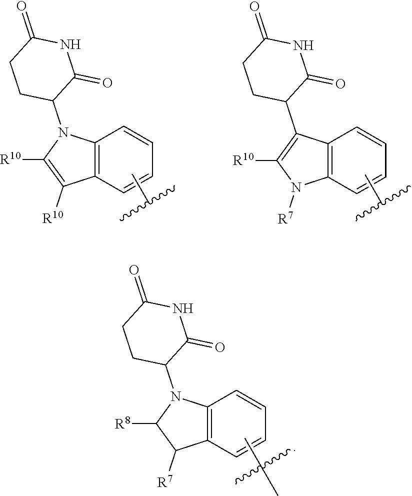

245. The compound of any one of claims 1-232, wherein Z is:

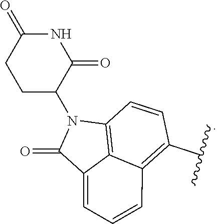 [SMILES: *c1ccc2c3c(cccc13)C(=O)N2C1CCC(=O)NC1=O]

246. The compound of any one of claims 1-232, wherein Z is:

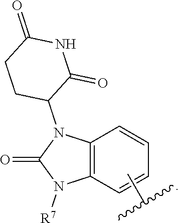

247. The compound of any one of claims 1-232, wherein Z is:

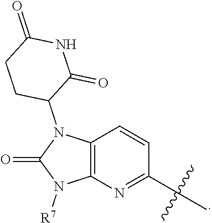

248. The compound of any one of claims 1-232, wherein Z is:

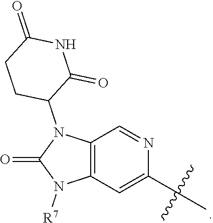

249. The compound of any one of claims 1-232, wherein Z is:

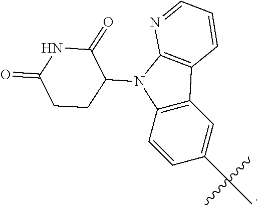 [SMILES: *c1ccc2c(c1)c1cccnc1n2C1CCC(=O)NC1=O]

250. The compound of any one of claims 1-232, wherein Z is:

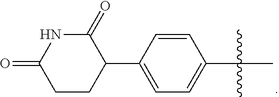 [SMILES: *c1ccc(C2CCC(=O)NC2=O)cc1]

251. The compound of any one of claims 1-250, wherein R⁷, if present, is hydrogen.

252. The compound of any one of claims 1-250, wherein R⁷, if present, is C1-C6 alkyl.

253. The compound of any one of claims 1-250, wherein R⁷, if present, is C1-C3 alkyl.

254. The compound of claim 253, wherein R⁷, if present, is methyl.

255. The compound of any one of claims 1-250, wherein R⁷, if present, is C1-C6 alkyl substituted with one group selected from hydroxyl, cyano and C1-C6 alkoxy.

256. The compound of any one of claims 1-250, wherein R⁷, if present, is C1-C6 haloalkyl.

257. The compound of any one of claims 1-250, wherein R⁷, if present, is C3-C6 cycloalkyl, or 4-6 membered heterocyclyl, —(CR^{A}R^{B})(4-12 membered heterocyclyl), or —(CR^{A}R^{B})(C3-C6 cycloalkyl).

258. The compound of claim 257, wherein each R^{A} and R^{B} are hydrogen.

259. The compound of any one of claims 1-258, wherein R⁸, if present, is hydrogen.

260. The compound of any one of claims 1-258, wherein R⁸, if present, is C1-C6 alkyl.

261. The compound of any one of claims 1-258 or 260, wherein R⁸, if present, is C1-C3 alkyl.

262. The compound of any one of claims 1-261, wherein q is 1.

263. The compound of any one of claims 1-262, wherein R⁹, if present, is hydrogen.

264. The compound of any one of claims 1-262, wherein R⁹, if present, is halogen.

265. The compound of any one of claims 1-262, wherein R⁹, if present, is cyano.

266. The compound of any one of claims 1-262, wherein R⁹, if present, is C1-C6 alkyl or C1-C6 haloalkyl.

267. The compound of any one of claims 1-262, wherein R⁹, if present, is C1-C6 alkoxy, C1-C5 cycloalkoxy, 5-10 membered heteroaryloxy, or phenoxy.

268. The compound of any one of claims 1-261, wherein q is 0.

269. The compound of any one of claims 1-268, wherein each R¹⁰ is hydrogen.

270. The compound of any one of claims 1-268, wherein one R¹⁰ is cyano, and the remaining R¹⁰, if present, are hydrogen.

271. The compound of any one of claims 1-268, wherein one R¹⁰ is halogen, and the remaining R¹⁰, if present, are hydrogen.

272. The compound of claim 271, wherein the halogen is fluoro.

273. The compound of any one of claims 1-268, wherein one R¹⁰ is C1-C6 alkyl, C1-C6 haloalkyl, or C3-C6 cycloalkyl, and the remaining R¹⁰, if present, are hydrogen.

274. The compound of claim 1, wherein the compound of Formula (I) is a compound of Formula (I-a): 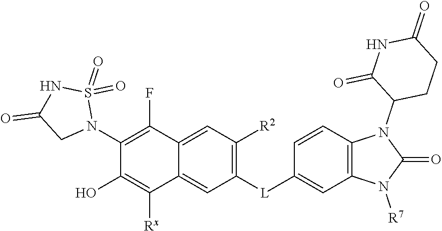
or a pharmaceutically acceptable salt thereof.

275. The compound of claim 1, wherein the compound of Formula (I) is a compound of Formula (I-b): 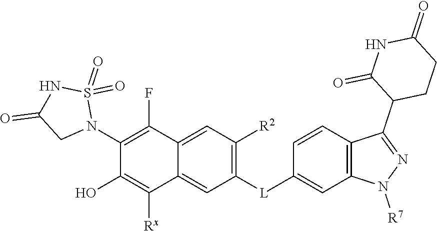
or a pharmaceutically acceptable salt thereof.

276. The compound of claim 1, wherein the compound of Formula (I) is a compound of Formula (I-c): 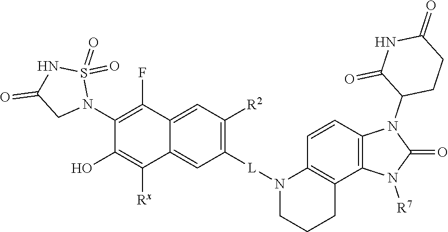
or a pharmaceutically acceptable salt thereof.

277. The compound of claim 1, wherein the compound of Formula (I) is a compound of Formula (I-d): 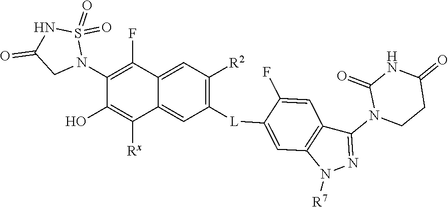
or a pharmaceutically acceptable salt thereof.

278. The compound of any one of claims 274-277, wherein R⁷ is C1-C3 alkyl.

279. The compound of any one of claims 274-277, wherein R⁷ is selected from the group consisting of methyl, ethyl, isopropyl, —(CH₂)₂OCH₃, and

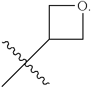 [SMILES: *C1COC1]

280. The compound of any one of claims 274-277, wherein R⁷ is methyl.

281. The compound of any one of claims 274-277, wherein R⁷ is ethyl.

282. The compound of claim 274-277, wherein R⁷ is hydrogen.

283. The compound of claim 1, wherein the compound of Formula (I) is a compound of Formula (I-e): 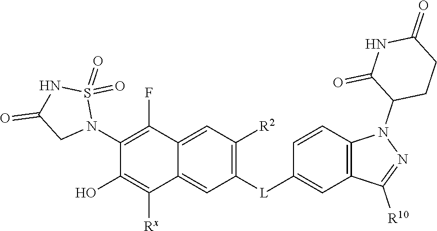
or a pharmaceutically acceptable salt thereof.

284. The compound of claim 283, wherein R¹⁰ is methyl.

285. The compound of claim 1, wherein the compound of Formula (I) is a compound of Formula (I-f): 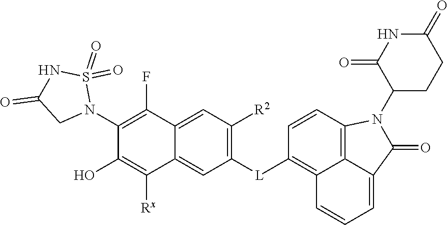
or a pharmaceutically acceptable salt thereof.

286. The compound of any one of claims 274-285, wherein R² is hydrogen.

287. The compound of any one of claims 274-285, wherein R² is halogen.

288. The compound of any one of claims 272-285, wherein R² is C1-C3 alkoxy, C3-C6 cycloalkoxy, C1-C3 haloalkoxy, or C3-C5 halocycloalkoxy.

289. The compound of any one of claims 272-285, wherein R² is C1-C3 alkyl or C3-C6 cycloalkyl.

290. The compound of claim 1, wherein the compound of Formula (I) is a compound of Formula (II-a): 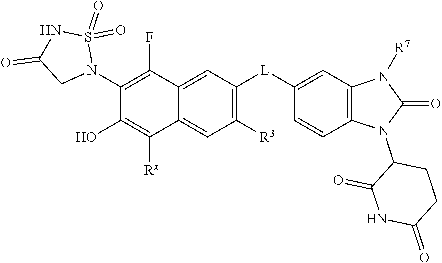
or a pharmaceutically acceptable salt thereof.

291. The compound of claim 1, wherein the compound of Formula (I) is a compound of Formula (II-b): 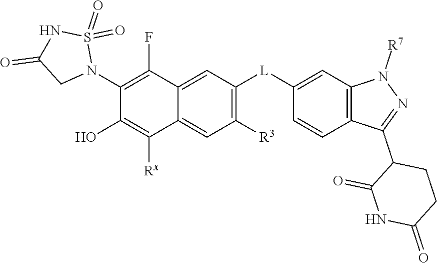
or a pharmaceutically acceptable salt thereof.

292. The compound of claim 1, wherein the compound of Formula (I) is a compound of Formula (II-c): 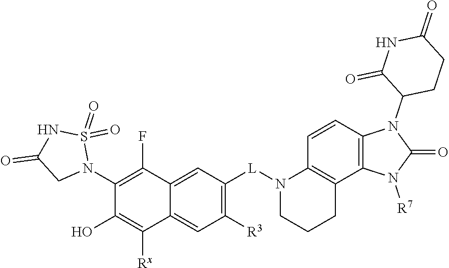
or a pharmaceutically acceptable salt thereof.

293. The compound of claim 1, wherein the compound of Formula (I) is a compound of Formula (II-d): 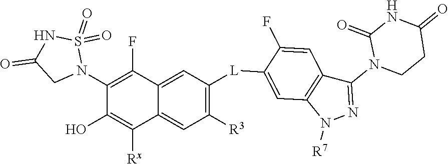
or a pharmaceutically acceptable salt thereof.

294. The compound of any one of claims 290-293, wherein R⁷ is C1-C3 alkyl.

295. The compound of any one of claims 290-293, wherein R⁷ is selected from the group consisting of methyl, ethyl, isopropyl, —(CH₂)₂OCH₃, and

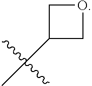 [SMILES: *C1COC1]

296. The compound of any one of claims 290-293, wherein R⁷ is methyl.

297. The compound of any one of claims 290-293, wherein R⁷ is hydrogen.

298. The compound of claim 1, wherein the compound of Formula (I) is a compound of Formula (II-e): 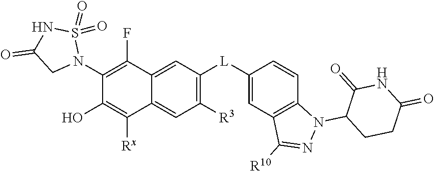
or a pharmaceutically acceptable salt thereof.

299. The compound of claim 298, wherein R¹⁰ is methyl.

300. The compound of claim 1, wherein the compound of Formula (I) is a compound of Formula (II-f): 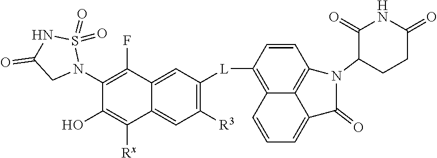
or a pharmaceutically acceptable salt thereof.

301. The compound of any one of claims 290-300, wherein R³ is hydrogen.

302. The compound of any one of claims 290-300, wherein R³ is halogen.

303. The compound of claim 302, wherein R³ is —F.

304. The compound of claim 302, wherein R³ is —Cl.

305. The compound of any one of claims 290-300, wherein R³ is C1-C3 alkoxy.

306. The compound of any one of claims 290-300, wherein R³ is C3-C6 cycloalkoxy.

307. The compound of any one of claims 290-300, wherein R³ is C1-C3 haloalkoxy.

308. The compound of any one of claims 290-300, wherein R³ is 3-C5 halocycloalkoxy.

309. The compound of any one of claims 290-300, wherein R³ is C1-C3 alkyl.

310. The compound of any one of claims 290-300, wherein R³ is C3-C6 cycloalkyl.

311. The compound of any one of claims 274-310, wherein R^{x} is hydrogen.

312. The compound of any one of claims 274-310, wherein R^{x} is halogen.

313. The compound of any one of claims 274-310 or 312, wherein R^{x} is fluoro.

314. The compound of any one of claims 274-313, wherein L is —U—V—W—X—Y—.

315. The compound of any one of claims 274-314, wherein U is —NR⁴(C═O)—, —(C═O)NR⁴—, or —(NR⁴)(C═O)(NR⁴)—; V is a bond, C1-C6 alkylene, or C3-C6 cycloalkylene; W is a bond; and X is a bond, C6-C10 arylene, or C1-C3 alkylene.

316. The compound of any one of claims 274-314, wherein U is —NR⁴(C═O)—, —(C═O)NR⁴—, or —(NR⁴)(C═O)(NR⁴)—; V is a bond, C1-C6 alkylene, 4-10 membered heterocyclyene, or C3-C6 cycloalkylene; W is a bond; and X is a bond.

317. The compound of any one of claims 274-314, wherein U is —NR⁴(C═O)—, —(C═O)NR⁴—, or —(NR⁴)(C═O)(NR⁴)—; V is a bond, C1-C6 alkylene, 4-10 membered heterocyclyene, or C3-C6 cycloalkylene; W is 4-10 membered heterocyclyene, C3-C6 cycloalkylene, C1-C3 alkylene optionally substituted with hydroxyl, —(NR⁴)R⁵—, —(NR⁴)(C═O)—, or —O—; and X is a bond, C6-C10 arylene, or R⁶ is C1-C3 alkylene.

318. The compound of any one of claims 315-317, wherein U is —NR⁴(C═O)—.

319. The compound of any one of claims 315-317, wherein U is —(C═O)NR⁴—.

320. The compound of any one of claims 315-317, wherein U is —(NR⁴)(C═O)(NR⁴)—.

321. The compound of any one of claims 274-320, wherein V is a bond.

322. The compound of any one of claims 274-320, wherein V is C1-C3 alkylene.

323. The compound of claim 322, wherein V is methylene or ethylene.

324. The compound of any one of claims 274-314, wherein U is —O—; V is C1-C6 alkylene, C3-C6 cycloalkylene, or 4-10-membered heterocyclylene; W is a bond —C(═O)—, —C(═O)R⁵—, —R⁵(C═O)—, —N(R⁴)—, —C(═O)NR⁴—, —NR⁴C(═O)—, or —NR⁴C(═O)R⁵—.

325. The compound of claim 324, wherein V is C1-C6 alkylene.

326. The compound of claim 324 or 325, wherein V is C1-C3 alkylene.

327. The compound of claim 326, wherein V is methylene or ethylene.

328. The compound of claim 324, wherein V is 4-10 membered heterocyclylene.

329. The compound of any one of claims 324-328, wherein W is —C(═O)—, —C(═O)R⁵—, a bond, or —C(═O)NR⁴—.

330. The compound of any one of claims 324-329, wherein W is —C(═O)R⁵—.

331. The compound of any one of claims 324-329, wherein W is a bond.

332. The compound of any one of claims 315-331, wherein R⁴ is hydrogen.

333. The compound of any one of claims 315-331, wherein R⁵ is C1-C3 alkylene.

334. The compound of any one of claims 315-331, wherein R⁵ is —CH₂—.

335. The compound of any one of claims 315-331, wherein R⁵ is C3-C7 cycloalkylene.

336. The compound of any one of claims 274-314, wherein U is a bond, C1-C3 alkylene, C2-C3 alkenylene, 4-10 membered heterocyclylene, or C2-C3 alkynylene; V is a bond or 4-10 membered heterocyclylene; W is a bond, —C(═O)—, or —C(═O)R⁵—; and X is a bond, C1-C3 alkylene or C6-C10 arylene.

337. The compound of claim 336, wherein U is a bond.

338. The compound of claim 336, wherein U is C2-C3 alkenylene.

339. The compound of claim 336, wherein U is 4-10 membered heterocyclylene.

340. The compound of any one of claims 336-339, wherein W is a bond.

341. The compound of any one of claims 336-339, wherein W is C(═O).

342. The compound of any one of claims 336-339, wherein W is C(═O)R⁵.

343. The compound of any one of claims 336-339, wherein X is a bond.

344. The compound of any one of claims 336-343, wherein X is C6-C10 arylene.

345. The compound of any one of claims 336-343, wherein X is C1-C3 alkylene.

346. The compound of any one of claims 274-314, wherein U is —NR⁴(C═O)—, —(C═O)NR⁴—, or —(NR⁴)(C═O)(NR⁴)—; V is C1-C6 alkylene; W is a bond; and X is a bond.

347. The compound of claim 345, wherein U is —NR⁴(C═O)—.

348. The compound of claim 346, wherein U is —(C═O)NR⁴—.

349. The compound of claim 346, wherein U is —(NR⁴)(C═O)(NR⁴)—.

350. The compound of any one of claims 346-349, wherein each R⁴ within U is hydrogen.

351. The compound of any one of claims 274-350, wherein Y is R⁶.

352. The compound of claim 351, wherein R⁶ is 4-12 membered heterocyclylene optionally substituted with 1-3 substituents independently selected from fluoro, hydroxyl, C1-C6 alkoxy, and C1-C6 alkyl.

353. The compound of any one of claims 351-352, wherein R⁶ is 4-8 membered heterocyclylene optionally substituted with 1-3 substituents independently selected from fluoro, hydroxyl, C1-C6 alkoxy, and C1-C6 alkyl.

354. The compound of claim 353, wherein R⁶ is selected from the group consisting of:

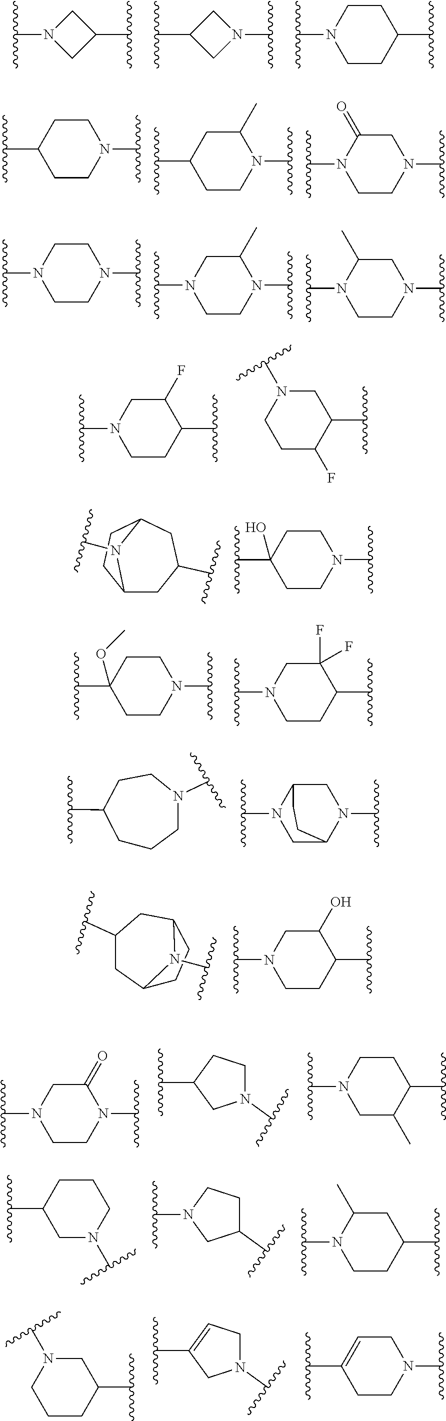

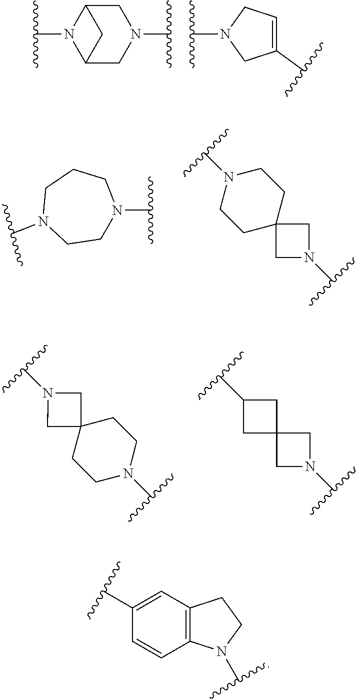

355. The compound of any one of claims 352-354, wherein R⁶ is

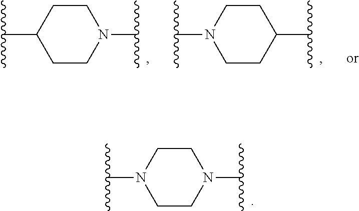

356. The compound of any one of claims 352-355, wherein R⁶ is

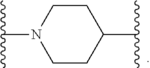 [SMILES: *C1CCN(*)CC1]

357. The compound of claim 351, wherein R⁶ is 4-8 membered heterocyclylene substituted with methyl, hydroxyl, methoxy, oxo, or 1 or 2 fluoros.

358. The compound of claim 351, wherein R⁶ is 7-12 membered bicyclic heterocyclylene.

359. The compound of claim 358, wherein R⁶ is 7-12 membered bicyclic spirocyclic heterocyclylene.

360. The compound of claim 358 or 359, wherein R⁶ is

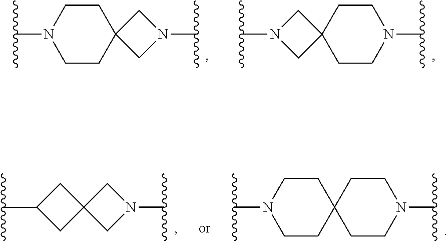

361. The compound of claim 351, wherein R⁶ is 5-10 membered heteroarylene.

362. The compound of claim 351 or 361, wherein R⁶ is 5-10 membered heteroarylene.

363. The compound of claim 362, wherein R⁶ is selected from the group consisting of:

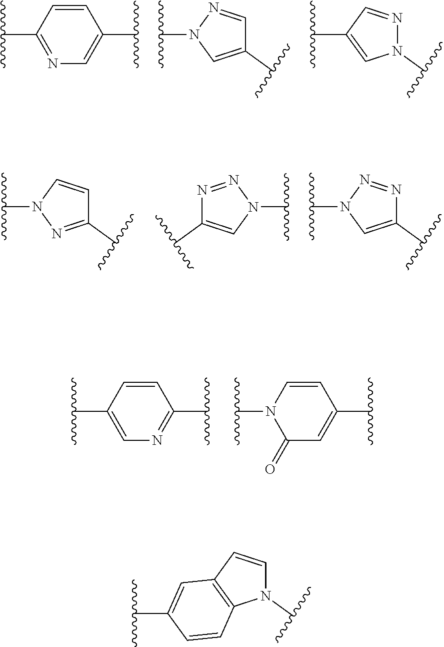

364. The compound of claim 351, wherein R⁶ is phenylene.

365. The compound of 351, wherein R⁶ is C1-C3 alkylene.

366. The compound of any one of claims 274-350, wherein Y is —R⁶(CR^{A}R^{B})ₚ-Q-; and p is 0.

367. The compound of claim 366, wherein Y is —R⁶NR⁴— or —R⁶O—.

368. The compound of claim 367, wherein Y is —R⁶NH.

369. The compound of claim 367, wherein Y is —R⁶O—.

370. The compound of any one of claims 274-350, wherein Y is R⁶(CR^{A}R^{B})ₚ-Q-; p is 1 or 2; and each R^{A} and R^{B} are hydrogen.

371. The compound of claim 370, wherein Y is —R⁶CH₂—O— or —R⁶CH₂—N(R⁴)—.

372. The compound of claim 371, wherein Y is —R⁶CH₂—O—.

373. The compound of claim 371, wherein Y is —R⁶CH₂—NH.

374. The compound of any one of claims 274-350, wherein Y is —R⁶(CR^{A}R^{B})ₚ-Q-, p is 1 or 2, and each R^{A} and R^{B} are independently hydrogen or C1-C3 alkyl; or one pair of R^{A} and R^{B}, together with the carbon atom to which they are attached, come together to form a C3-C4 cycloalkyl, and each remaining R^{A} and R^{B}, if present, are hydrogen.

375. The compound of claim 374, wherein the —(CR^{A}R^{B})ₚ-Q- portion of Y is selected from the group consisting of:

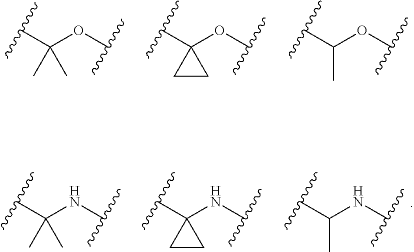

376. The compound of any one of claims 274-350, wherein Y is —R⁶C(═O)(CR^{A}R^{B})-Q-; and each R^{A} and R^{B} are independently hydrogen, fluoro, or C—C3 alkyl.

377. The compound of claim 376, wherein the —(CR^{A}R^{B})ₚ-Q- portion of Y is selected from the group consisting of:

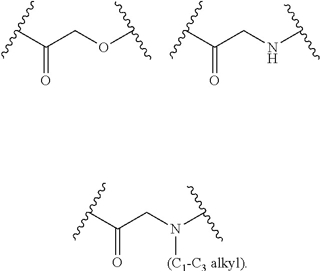

378. The compound of any one of claims 366-377, wherein R⁶ is 4-12 membered heterocyclylene optionally substituted with 1-3 substituents independently selected from fluoro, hydroxyl, C1-C6 alkoxy, and C1-C6 alkyl.

379. The compound of any one of claims 366-377, wherein R⁶ is 4-8 membered heterocyclylene optionally substituted with 1-3 substituents independently selected from fluoro, hydroxyl, C1-C6 alkoxy, and C1-C6 alkyl.

380. The compound of claim 366-377, wherein R⁶ is selected from the group consisting of:

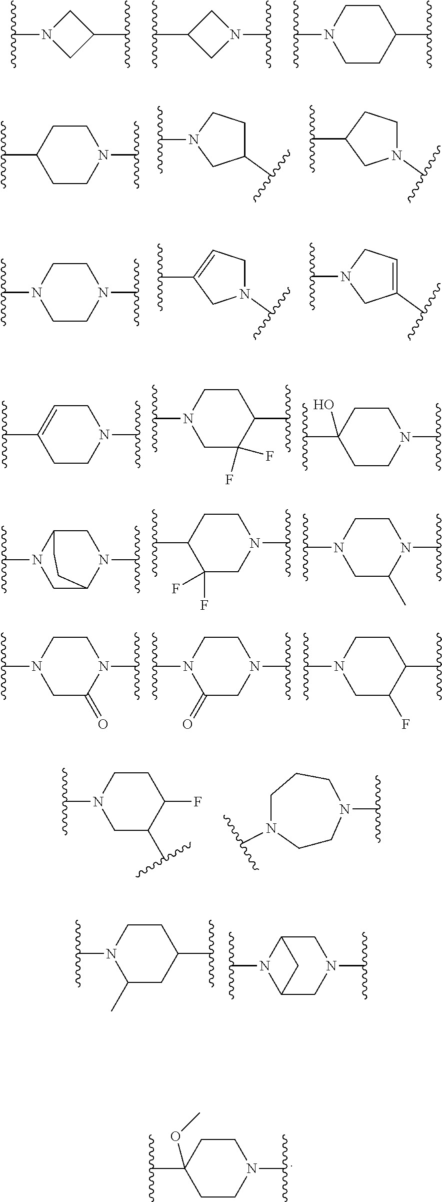

381. The compound of any one of claims 366-377, wherein R⁶ is

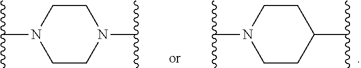 [SMILES: *N1CCN(*)CC1 ; *C1CCN(*)CC1]

382. The compound of any one of claims 366-377, wherein R⁶ is

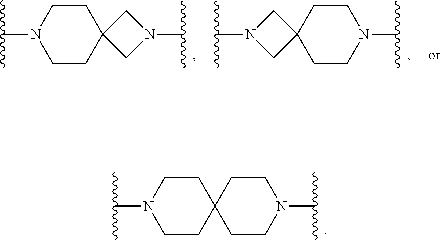

383. The compound of any one of claims 366-377, wherein R⁶ is 7-12 membered bicyclic heterocyclylene.

384. The compound of claim 383, wherein R⁶ is 7-12 membered bicyclic spirocyclic heterocyclylene.

385. The compound of claim 383 or 384, wherein R⁶ is

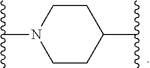 [SMILES: *C1CCN(*)CC1]

386. The compound of any one of claims 366-377, wherein R⁶ is C6-C10 arylene.

387. The compound of any one of claims 366-377 or 386, wherein R⁶ is phenylene.

388. The compound of any one of claims 366-377, wherein R⁶ is 5-10 membered heteroarylene.

389. The compound of any one of claims 366-377 or 388, wherein R⁶ is 5-6 membered heteroarylene.

390. The compound of any one of claims 366-377 or 388-389, wherein R⁶ is 5-membered heteroarylene.

391. The compound of claim 390, wherein R⁶ is triazolylene or pyrazolylene.

392. The compound of claim 391, wherein R⁶ is selected from the group consisting of:

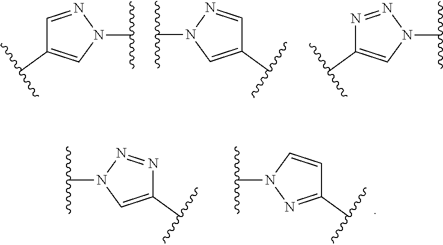

393. The compound of any one of claims 366-377 or 388-389, wherein R⁶ is 6-membered heteroarylene.

394. The compound of claim 393, wherein R⁶ is pyridinylene.

395. The compound of claim 394, wherein R⁶ is selected from the group consisting of:

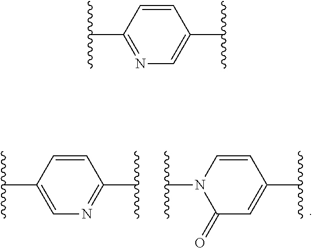

396. The compound of claim 1, wherein:
R¹ is fluoro;
R^{x} is hydrogen;
R² is hydrogen;
R³ is -L-Z;
Z is 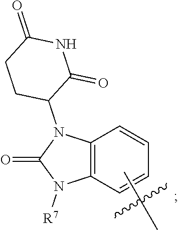
and
R⁷ is hydrogen or C1-C6 alkyl.

397. The compound of claim 1, wherein:
R¹ is fluoro;
R^{x} is hydrogen;
R² is -L-Z;
R³ is hydrogen;
Z is 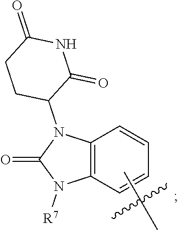
and
R⁷ is hydrogen or C1-C6 alkyl.

398. The compound of claim 396 or 397, wherein:
U is —(NR⁴)C═O)—, —(C═O)NR⁴—, or —(NR⁴)(C═O)(NR⁴)—;
V is a bond, C1-C6 alkylene, or 4-6-membered heterocyclylene optionally substituted with methyl, hydroxyl, methoxy, or 1 or 2 fluoros;
W is a bond or C1-C3 alkylene;
X is a bond or C1-C3 alkylene;
Y is R⁶;
R⁶ is C1-C3 alkylene, C3-C7 cycloalkylene, 4-12 membered heterocyclylene, C6-C10 arylene, or 5-10 membered heteroarylene; and
R⁴ is hydrogen or C1-C6 alkyl.

399. The compound of any one of claims 1 or 396-397, wherein:
U is —(NR⁴)C═O)—, —(C═O)NR⁴—, or —(NR⁴)(C═O)(NR⁴)—;
V is a bond or 4-6-membered heterocyclylene optionally substituted with methyl, hydroxyl, methoxy, or 1 or 2 fluoros;
W is a bond or C1-C3 alkylene;
X is a bond or C1-C3 alkylene;
Y is R⁶;
R⁶ is 4-8 membered heterocyclylene, phenyl, or 5-6 membered heteroarylene; and
R⁴ is hydrogen or C1-C6 alkyl.

400. The compound of any one of claims 1 or 396-399, wherein W and X are bonds.

401. The compound of any one of claims 1 or 396-399, wherein R⁶ is piperidinyl, piperazinyl, phenyl, pyridinyl, or pyridonyl.

402. The compound of any one of claims 1 or 396-399, wherein W is a bond and R⁴ is hydrogen.

403. The compound of any one of claims 1 or 396-399, wherein U is —(NR⁴)C═O)—, V is a bond, W is a bond, X is a bond, and Y is R⁶.

404. The compound of claim 403, wherein R⁴ is hydrogen or methyl; and R⁶ is 5-6 membered heterocyclylene, phenyl, or 5-6 membered heteroarylene.

405. The compound of claim 403 or 404, wherein R⁶ is piperidinyl, piperazinyl, phenyl, pyridinyl, or pyridonyl.

406. The compound of claim 1, wherein the compound of Formula (I) is selected from the compounds described in Table 1, or a pharmaceutically acceptable salt thereof.

407. The compound of any one of claims 1-406, wherein the compound of Formula (I) is present in the form of a pharmaceutically acceptable salt.

408. A pharmaceutical composition comprising a compound of any one of claims 1-406, or a pharmaceutically acceptable salt thereof.

409. A method for treating cancer in a subject in need thereof, comprising administering to the subject a therapeutically effective amount of a compound of any one of claims 1-406, or a pharmaceutically acceptable salt thereof, or a pharmaceutical composition according to claim 408.

410. A method for inhibiting mammalian cell proliferation, comprising contacting the mammalian cell with an effective amount of a compound of any one of claims 1-406, or a pharmaceutically acceptable salt thereof.

411. A method for decreasing levels of a protein in a mammalian cell, comprising contacting the mammalian cell with an effective amount of a compound of any one of claims 1-406, or a pharmaceutically acceptable salt thereof; wherein the protein is PTPN1, PTPN2, or a combination thereof.

412. The method of claim 410 or 411, wherein the contacting occurs in vivo.

413. The method of claim 410 or 411, wherein the contacting occurs in vitro.

414. The method of any one of claims 410-413, wherein the mammalian cell is a mammalian cancer cell.

415. A method for inhibiting metastasis in a subject having a particular cancer in need of such treatment, comprising administering to the subject a therapeutically effective amount of a compound of any one of claims 1-406, or a pharmaceutically acceptable salt thereof, or the pharmaceutical composition of claim 408.

416. A method for treating a metabolic disease in a subject in need thereof, comprising administering to the subject a therapeutically effective amount of a compound of any one of claims 1-406, or a pharmaceutically acceptable salt thereof, or the pharmaceutical composition of claim 408.

417. The method of claim 416, wherein the metabolic disease is NAFLD, NASH, type 2 diabetes, or a combination of any of the foregoing.

418. The method of claim 416 or 417, wherein the metabolic disease is type 2 diabetes.

419. A method for decreasing BMI in a subject in need thereof, comprising administering to the subject a therapeutically effective amount of a compound of any one of claims 1-406, or a pharmaceutically acceptable salt thereof, or the pharmaceutical composition of claim 408.

420. A method for inhibiting weight gain in a subject in need thereof, comprising administering to the subject a therapeutically effective amount of a compound of any one of claims 1-405, or a pharmaceutically acceptable salt thereof, or the pharmaceutical composition of claim 408.

421. The method of any one of claims 419-420 wherein the subject has an average BMI of between about 25 and about 45 prior to initiation of treatment with a compound of Formula (I), or a pharmaceutically acceptable salt thereof.

422. A method for increasing proliferation of mammalian T-cells in the presence of T-cell receptor stimulation, comprising contacting a mammalian thymus cell with an effective amount of a compound of any one of claims 1-406, or a pharmaceutically acceptable salt thereof; wherein the protein is PTPN1, PTPN2, or a combination thereof.

423. A method for activating mammalian T-cells in the presence of T-cell receptor stimulation, comprising contacting the mammalian T-cell with an effective amount of a compound of any one of claims 1-406, or a pharmaceutically acceptable salt thereof; wherein the protein is PTPN1, PTPN2, or a combination thereof.

424. The method of claim 422 or 423, wherein the contacting occurs in vivo.

425. The method of claim 422 or 423, wherein the contacting occurs in vitro.
